(19) United States
(12) Patent Application Publication
(10) Pub. No.: US 2026/0250299 A1
(43) Pub. Date: Aug. 27, 2026

(54) TRICYCLIC QUINOLONE BCL6 BIFUNCTIONAL DEGRADERS

(71) Applicant: Treeline Biosciences, Inc., Watertown, MA (US)

(72) Inventors: Kevin M. Oberg, San Diego, CA (US); Joshua D. Hansen, San Diego, CA (US); Matthew D. Correa, San Diego, CA (US); Tami J. Marrone, Carlsbad, CA (US); Matthew H. McNeill, San Clemente, CA (US); Mark A. Nagy, Encinitas, CA (US)

(21) Appl. No.: 19/659,274

(22) Filed: Apr. 27, 2026

Related U.S. Application Data
(63) Continuation of application No. 18/329,225, filed on Jun. 5, 2023.
(60) Provisional application No. 63/349,415, filed on Jun. 6, 2022.
(60) Provisional application No. 63/349,420, filed on Jun. 6, 2022.
(60) Provisional application No. 63/356,388, filed on Jun. 28, 2022.
(60) Provisional application No. 63/356,390, filed on Jun. 28, 2022.
(60) Provisional application No. 63/414,409, filed on Oct. 7, 2022.
(60) Provisional application No. 63/414,362, filed on Oct. 7, 2022.
(60) Provisional application No. 63/414,349, filed on Oct. 7, 2022.
(60) Provisional application No. 63/414,418, filed on Oct. 7, 2022.
(60) Provisional application No. 63/420,385, filed on Oct. 28, 2022.
(60) Provisional application No. 63/420,398, filed on Oct. 28, 2022.
(60) Provisional application No. 63/444,769, filed on Feb. 10, 2023.
(60) Provisional application No. 63/444,792, filed on Feb. 10, 2023.
(60) Provisional application No. 63/497,061, filed on Apr. 19, 2023.
(60) Provisional application No. 63/501,080, filed on May 9, 2023.

(51) Int. Cl. C07D 498/04; A61K 31/553; A61K 45/06; A61K 47/55; A61P 35/00; C07D 519/00

(52) U.S. Cl. CPC C07D 498/04; A61K 31/553; A61K 45/06; A61K 47/55; A61P 35/00; C07D 519/00

(57) ABSTRACT

This disclosure provides compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, that induce degradation of a BCL6 protein. These compounds are useful, for example, for treating a cancer in a subject (e.g., a human). This disclosure also provides compositions containing the compounds provided herein as well as methods of using and making the same.

### CROSS-REFERENCE TO RELATED APPLICATIONS

This application is a continuation application of U.S. patent application Ser. No. 18/329,225, filed Jun. 5, 2023, which claims priority to U.S. Provisional Application Ser. No. 63/349,415, filed Jun. 6, 2022; 63/349,420, filed Jun. 6, 2022; 63/356,388, filed Jun. 28, 2022; 63/356,390, filed Jun. 28, 2022; 63/414,409, filed Oct. 7, 2022; 63/414,362, filed Oct. 7, 2022; 63/414,349, filed Oct. 7, 2022; 63/414,418, filed Oct. 7, 2022; 63/420,385, filed Oct. 28, 2022; 63/420,398, filed Oct. 28, 2022; 63/444,769, filed Feb. 10, 2023; 63/444,792, filed Feb. 10, 2023; 63/497,061, filed Apr. 19, 2023; and 63/501,080, filed May 9, 2023; each of which is incorporated by reference in its entirety herein.

### DESCRIPTION OF THE TEXT FILE SUBMITTED ELECTRONICALLY

This application contains a Sequence Listing which has been submitted electronically in XML format. The Sequence Listing XML is incorporated herein by reference. Said XML file, created on May 26, 2023, is named TLS-039US-SL.xml and is 2,663 bytes in size.

### TECHNICAL FIELD

This disclosure provides compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, that induce degradation of a BCL6 protein. These compounds are useful, for example, for treating cancer in a subject (e.g., a human). This disclosure also provides compositions containing the compounds provided herein as well as methods of using and making the same.

### BACKGROUND

B-cell lymphoma 6 (BCL6) protein is a transcriptional repressor involved in the formation and maintenance of germinal centers (GCs) within lymphoid follicles. It controls the functions of the GC and coordinates the activities of signaling mediators in the maturation of GC B cells. There are over 1000 known or putative BCL6 target genes, including MYC, BCL2, genes related to DNA damage response (e.g., ATR, TP53), and cell cycle checkpoint control (e.g., CDKN1A, CDKN1B).

BCL6 is expressed in the dark zone cells of GCs, where somatic hypermutation is allowed to occur to generate high-affinity B-cell receptors. Overexpression or loss of control of BCL6, for example by translocation, can permit maintenance of the pro-hypermutation functions and abrogation of the antitumor functions of BCL6.

### SUMMARY

Provided herein are compounds of Formula (I):

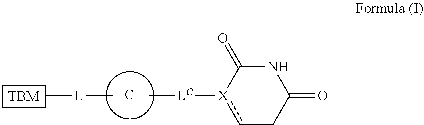

or pharmaceutically acceptable salts thereof, wherein:
TBM is (T1):

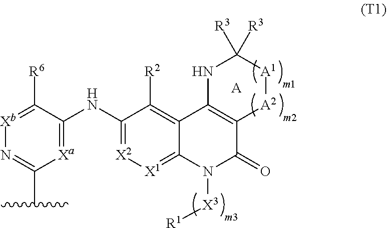

X¹ is selected from the group consisting of N and CR²;
X² is CH;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
m3 is 0 or 1;
X³ is C₁₋₃ alkylene optionally substituted with 1-3 R^{c};
R¹ is selected from the group consisting of: H and C₃₋₆ cycloalkyl;
m1 is 2; each A¹ is independently CH₂, CHR⁴, or CR⁴R⁴;
m2 is 1; A² is —O—;
one R³ is selected from the group consisting of:
(i) C₃₋₆ cycloalkyl optionally substituted with 1-3 R^{g}, and
(ii) C₁₋₃ alkyl optionally substituted with 1-3 —F; and
the other R³ is H;
each R⁴ is independently selected from the group consisting of: H, Ra, and R^{b};
X^{a} is selected from the group consisting of: N, CH, and CF;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1} L^{A3}, and L^{A4} provided that 1-3 occurrences of L^{A} is L^{A4};

### (BB)

n1 is an integer from 0 to 20; and
each L^{A} is independently selected from the group consisting of: L^{A}1 and L^{A3};
each L^{A1} is independently selected from the group consisting of: —CH₂—, —CHR^{L}—, and —C(R^{L})₂-;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b}; provided that L does not contain any O—O, N—O, N—N, N—S(O)o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{e};
Ring C is selected from the group consisting of:

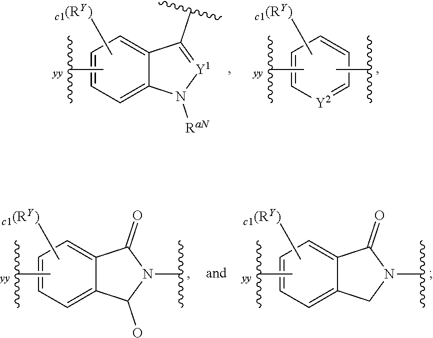

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than 0; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)C₁₋₆ haloalkyl;
(i) C(═O)OH;
(j) C(═O)OC₁₋₆ alkyl;
(k) C(═O)OC₁₋₆ haloalkyl;
(l) C(═O)N(R^{f})₂;
(m) S(O)₀₋₂(C₁₋₆ alkyl);
(n) S(O)₀₋₂(C₁₋₆ haloalkyl);
(o) S(O)₁₋₂N(Rf)₂; and
(p) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(Rf)₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R¹ is independently selected from the group consisting of: H and C₁₋₆ alkyl optionally substituted with 1-3 R^{h};
each R⁹ is independently selected from the group consisting of: R^{h}, oxo, C₁₋₃ alkyl, and C₁₋₃ haloalkyl; and each R^{h} is independently selected from the group consisting of: halo, cyano, —OH, —(C₀₋₃ alkylene)-C1-6 alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

Also provided herein are pharmaceutical compositions comprising a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))), or a pharmaceutically acceptable salt thereof, and a pharmaceutically acceptable carrier.

Provided herein are methods for treating cancer in a subject in need thereof, the methods comprising administering to the subject a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))), or a pharmaceutically acceptable salt thereof, or a pharmaceutical composition as provided herein.

Also provided herein are BCL6 proteins non-covalently bound with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))), or a pharmaceutically acceptable salt thereof.

Also provided herein are ternary complexes comprising a BCL6 protein, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))), or a pharmaceutically acceptable salt thereof, and a CRBN protein, or a portion thereof.

To facilitate understanding of the disclosure set forth herein, a number of additional terms are provided. Generally, the nomenclature used herein and the laboratory procedures in organic chemistry, medicinal chemistry, and pharmacology described are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In the case of conflict between the present disclosure and any content incorporated by reference, the present disclosure controls.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))), or pharmaceutically acceptable salts thereof, that induce degradation of a BCL6 protein. These compounds are useful, e.g., for treating a cancer.

This disclosure also provides compositions containing the compounds provided herein as well as methods of using and making the same.

Upon antigen challenge, germinal centers (GCs) are formed in lymphoid follicles, and B-cells in the dark zone of GCs undergo rapid proliferation and somatic hypermutation, both of their immunoglobin variable genes to generate high-affinity B-cell receptors, as well as of other genes including BCL6. BCL6 is often considered to be a ‘master regulator’ of the GC reaction. In some cancers, BCL6 can be mutated, translocated, and/or BCL6 expression can be upregulated. See, e.g., Leeman-Neill and Bhagat, Expert Opinion on Therapeutic Targets 22.2 (2018): 143-152; Mlynarczyk and Melnick. Immunological Reviews 288.1 (2019): 214-239.

The BCL6 protein has multiple domains, including a BTB domain, an RD2 domain, and a DNA binding domain. The N-terminal BTB domain is the site of homodimerization of BCL6, and the interface of the monomers forms the “lateral groove”, which is a binding site for endogenous co-repressors of BCL6, such as SMRT, NCOR, and BCOR. See, e.g., Cardenas, Mariano G., et al. Clinical Cancer Research 23.4 (2017): 885-893.

Compounds that induce degradation of a target protein are sometimes referred to as heterobifunctional compounds, PROTACs, or degraders. Such compounds generally include a moiety that binds to the target protein and a moiety that binds to a ubiquitin E3 ligase (sometimes referred to as an E3 ligase or simply an E3), these two moieties being optionally separated by a linker. To induce degradation, heterobifunctional compounds are believed to induce formation of a ternary complex between the target protein, the compound, and an E3 ligase. Formation of the ternary complex is then followed by ubiquitination of the target protein and degradation of the ubiquitinated target protein by a proteosome. Several E3 ligases have been used as the partner E3 ligase for heterobifunctional degraders. Herein, the cereblon (CRBN) E3 ligase (also referred to herein as a CRBN protein) is used.

A degradation approach for a target protein can have potential advantages compared to, e.g., small molecule inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule inhibition in some cases, as degradation can impair a scaffolding function of a target protein, whereas a small molecule might not. It is also generally believed that for formation of a ternary complex, high affinity to the target protein is not always required.

Heterobifunctional compounds are further described in, for example, International Publication Nos. WO 2021/077010 and WO 2022/221673; McCoull, William, et al., ACS Chemical Biology 13.11 (2018): 3131-3141; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12.

### Compound Embodiments

Provided herein are compounds of Formula (I):

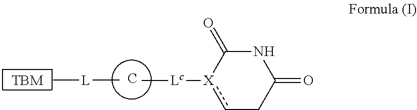

or pharmaceutically acceptable salts thereof, wherein:
TBM is (T1):

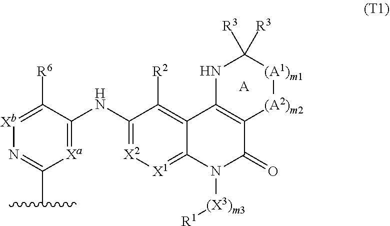

X¹ is selected from the group consisting of N and CR²;
X² is CH;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
m3 is 0 or 1;
X³ is C₁₋₃ alkylene optionally substituted with 1-3 R^{c};
R¹ is selected from the group consisting of: H and C₃₋₆ cycloalkyl;
m1 is 2; each A¹ is independently CH₂, CHR⁴, or CR⁴R⁴;
m2 is 1; A² is —O—;
one R³ is selected from the group consisting of:
(i) C₃₋₆ cycloalkyl optionally substituted with 1-3 R⁹, and
(ii) C₁₋₃ alkyl optionally substituted with 1-3 —F; and
the other R³ is H;
each R⁴ is independently selected from the group consisting of: H, R^{a}, and R^{b};
X^{a} is selected from the group consisting of: N, CH, and CF;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1} L^{A3}, and L^{A4} provided that 1-3 occurrences of L^{A} is L^{A4};

### (BB)

n1 is an integer from 0 to 20; and
each L^{A} is independently selected from the group consisting of: L^{A}1 and L^{A3};
each L^{A}1 is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{c};
Ring C is selected from the group consisting of:

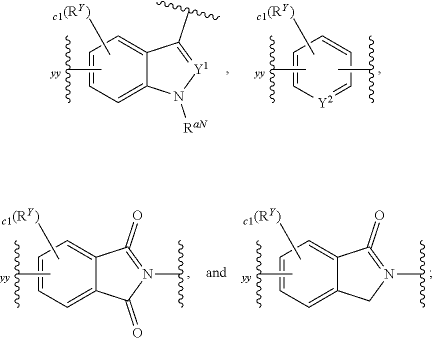

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)C₁₋₆ haloalkyl;
(i) C(═O)OH;
(j) C(═O)OC₁₋₆ alkyl;
(k) C(═O)OC₁₋₆ haloalkyl;
(1) C(═O)N(R^{a})₂;
(m) S(O)₀₋₂(C₁₋₆ alkyl);
(n) S(O)₀₋₂(C₁₋₆ haloalkyl);
(o) S(O)₁₋₂N(Rf)₂; and
(p) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{e} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(Rf)₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R¹ is independently selected from the group consisting of: H and C₁₋₆ alkyl optionally substituted with 1-3 R^{h};
each R⁹ is independently selected from the group consisting of: R^{h}, oxo, C₁₋₃ alkyl, and Cₙ₁3 haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments, Ring C is selected from the group consisting of:

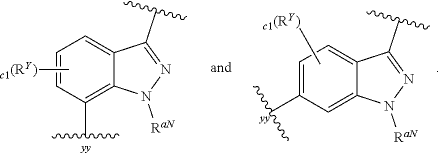

In some embodiments, c1 is 0 or 1; and R^{Y} is selected from the group consisting of halo (e.g., —F) and C₁₋₃ alkyl optionally substituted with 1-3 F, and R^{aN} is C₁₋₃ alkyl.

In some embodiments, Ring C is selected from the group consisting of:

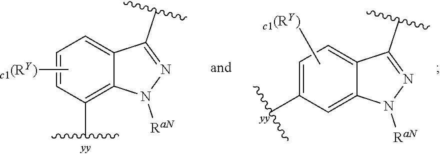

c1 is 0 or 1; and R^{Y} is selected from the group consisting of halo (e.g., —F) and C₁₋₃ alkyl optionally substituted with 1-3 F, and R^{aN} is C₁₋₃ alkyl.

In some embodiments, Ring C is

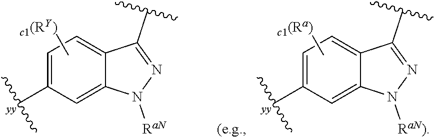

In some embodiments, c1 is 0. In some embodiments, c1 is 1; and R^{Y} is halo (e.g., —F). In some embodiments, R^{aN} is C₁₋₃ alkyl (e.g., methyl).

In some embodiments, Ring C is

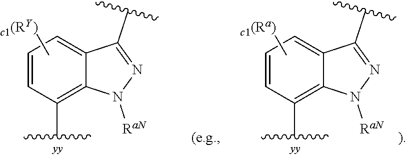

In some embodiments, c1 is 0. In some embodiments, c1 is 1; and R¹ is halo (e.g., —F). In some embodiments, R^{aN} is C₁₋₃ alkyl (e.g., methyl).

In some embodiments, Ring C is

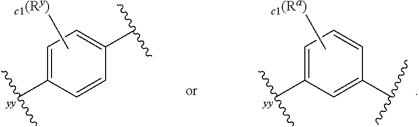

In some embodiments, c1 is 0. In some embodiments, c1 is 1; and R^{Y} is halo (e.g., —F).

In some embodiments, Ring C is

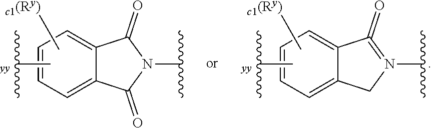

some embodiments, c1 is 0. In some embodiments, c1 is 1; and R^{Y} is halo (e.g., —F).

In some embodiments, c1 is 0. In some embodiments, c1 is 1; and R^{Y} is halo (e.g., —F). In some embodiments, R^{aN} is C₁₋₃ alkyl (e.g., methyl).

In some embodiments, X is CH.

In some embodiments, L^{C} is a bond.

In some embodiments, the

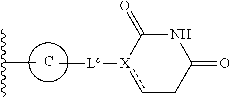

moiety is selected from the group consisting of the moieties delineated in Table CM-1b:

TABLE CM-1b

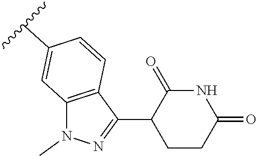 [SMILES: *c1ccc2c(C3CCC(=O)NC3=O)nn(C)c2c1]

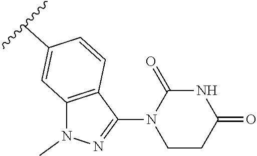 [SMILES: *c1ccc2c(N3CCC(=O)NC3=O)nn(C)c2c1]

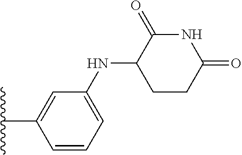 [SMILES: *c1cccc(NC2CCC(=O)NC2=O)c1]

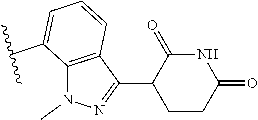 [SMILES: *c1cccc2c(C3CCC(=O)NC3=O)nn(C)c12]

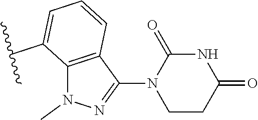 [SMILES: *c1cccc2c(N3CCC(=O)NC3=O)nn(C)c12]

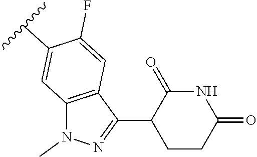 [SMILES: *c1cc2c(cc1F)c(C1CCC(=O)NC1=O)nn2C]

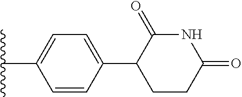 [SMILES: *c1ccc(C2CCC(=O)NC2=O)cc1]

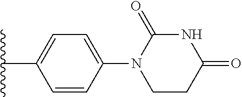 [SMILES: *c1ccc(N2CCC(=O)NC2=O)cc1]

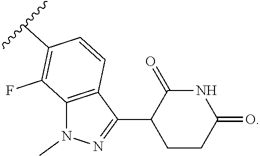 [SMILES: *c1ccc2c(C3CCC(=O)NC3=O)nn(C)c2c1F]

In some embodiments, the

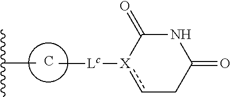

moiety is selected from the group consisting of the moieties delineated in Table CM-1a:

TABLE CM-1a

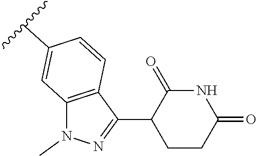 [SMILES: *c1ccc2c(C3CCC(=O)NC3=O)nn(C)c2c1]

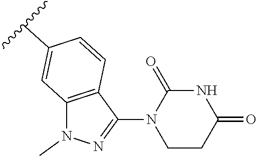 [SMILES: *c1ccc2c(N3CCC(=O)NC3=O)nn(C)c2c1]

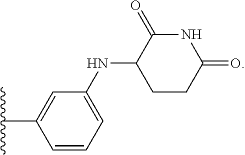 [SMILES: *c1cccc(NC2CCC(=O)NC2=O)c1]

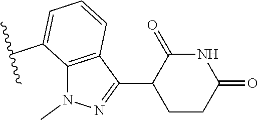 [SMILES: *c1cccc2c(C3CCC(=O)NC3=O)nn(C)c12]

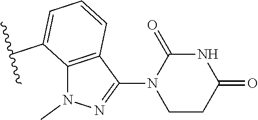 [SMILES: *c1cccc2c(N3CCC(=O)NC3=O)nn(C)c12]

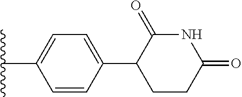 [SMILES: *c1ccc(C2CCC(=O)NC2=O)cc1]

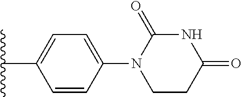 [SMILES: *c1ccc(N2CCC(=O)NC2=O)cc1]

In some embodiments,

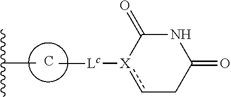

moiety is

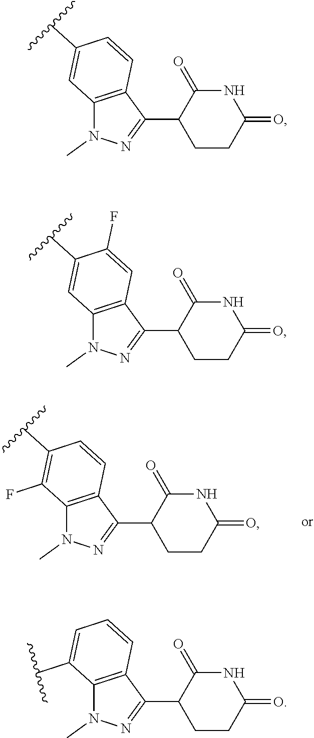 [SMILES: *c1ccc2c(C3CCC(=O)NC3=O)nn(C)c2c1 ; *c1cc2c(cc1F)c(C1CCC(=O)NC1=O)nn2C ; *c1ccc2c(C3CCC(=O)NC3=O)nn(C)c2c1F ; "or" ; *c1cccc2c(C3CCC(=O)NC3=O)nn(C)c12]

In some embodiments,

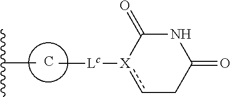

moiety is

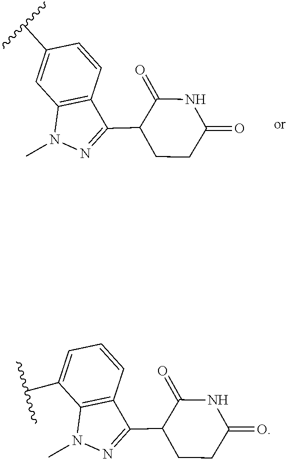 [SMILES: *c1ccc2c(C3CCC(=O)NC3=O)nn(C)c2c1 ; *c1cccc2c(C3CCC(=O)NC3=O)nn(C)c12]

In some embodiments,

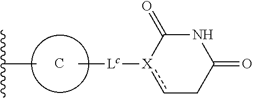

moiety is

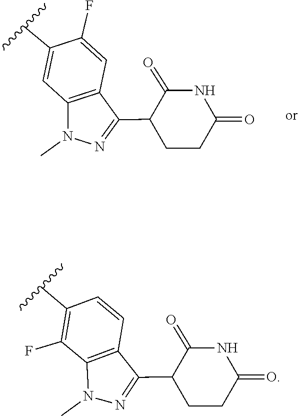 [SMILES: *c1cc2c(cc1F)c(C1CCC(=O)NC1=O)nn2C ; *c1ccc2c(C3CCC(=O)NC3=O)nn(C)c2c1F]

In some embodiments, -(A¹)ₘ₁ is —C(R⁴R⁴)—CH₂—* (e.g., —CF₂—CH₂—*), wherein * represents the point of attachment to -(A²)ₘ₂-.

In some embodiments, one R³ is C₃₋₆ cycloalkyl; and the other R³ is H. In some embodiments, one R³ is cyclopropyl; and the other R³ is H.

In some embodiments, one R³ is cyclopropyl; the other R³ is H; and -(A¹)ₘ₁- is —CF₂—CH₂—*, wherein * represents the point of attachment to -(A²)ₘ₂-.

In some embodiments, X² is CH. In some embodiments, X¹ is CH.

In some embodiments, the carbon atom to which each R³ is attached has (S)-stereochemical configuration.

In some embodiments, Xa is N; and X^{b} is CH.

In some embodiments, X^{a} is CH; and X^{b} is CH.

In some embodiments, R⁶ is halo (e.g., —F, —Cl, —Br) or CN. For example, R⁶ can be —Cl, —F, or CN.

In some embodiments, R⁶ is halo (e.g., —F, —Cl, —Br). In some embodiments, R⁶ is —F. In some embodiments, R⁶ is —Cl. In some embodiments, R⁶ is —Br.

In some embodiments, R⁶ is CN.

In some embodiments, each R² is H.

In some embodiments, m3 is 1; and X³ is C₁₋₃ alkylene (e.g., methylene, ethylene, or isopropylene).

In some embodiments, m3 is 0.

In some embodiments, R¹ is H.

In some embodiments, m3 is 1; X³ is C₁₋₃ alkylene optionally substituted with 1-3 R^{c}; and R¹ is H. In some embodiments, m3 is 1; X³ is C₁₋₃ alkylene; and R¹ is H.

In some embodiments, —(X³)ₘ₃—R¹ is methyl, ethyl, or isopropyl (e.g., methyl).

In some embodiments, TBM is

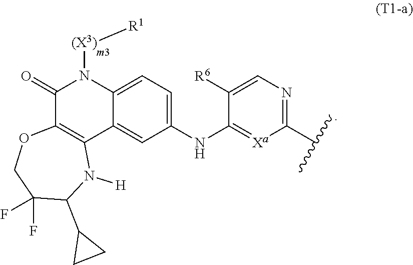

For example, TBM can be

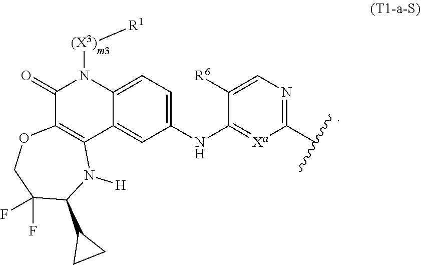

In some embodiments of (T1-a), m3 is 1; X³ is C₁₋₃ alkylene; and R¹ is H.

In some embodiments of (T1-a), —(X³)ₘ₃—R¹ is methyl, ethyl, or isopropyl. For example, —(X³)ₘ₃—R¹ can be methyl.

In some embodiments of (T1-a), X^{a} is CH. In some embodiments, X^{a} is N.

In some embodiments of (T1-a), R⁶ is —F or —Cl.

In some embodiments of (T1-a), Xa is N or CH; R⁶ is —F or —Cl; m3 is 1; X³ is C₁₋₃ alkylene; and R¹ is H.

In some embodiments, L is -(L^{A})ₙ₁-, wherein L^{A} and n1 are defined according to (AA).

In some embodiments, n1 is an integer from 1 to 5. In some embodiments, n1 is an integer from 2 to 4 (e.g., 2 or 3).

In some embodiments, L is selected from the group consisting of: -L^{A4}-L^{A}1-L^{A4}-_{bb}; -L^{A4}-L^{A4}-_{bb}; -L^{A4}-L^{A1}-L^{A1}-L^{A4}-_{bb}; -L^{A4}-L^{A3}-L^{A4}-_{bb}; and -L^{A4}-L^{A1}-L^{A4}-L^{A3}bb, wherein bb represents the point of attachment to Ring C.

In some embodiments, L is -L^{A4}-L^{A1}-L^{A4}-_{bb}. In some embodiments, L is -L^{A4}-L^{A1}-L^{A4}-bb, and L^{A1} is —CH₂—, —CHMe-, —CMe₂-, or —CH(OH)—. In some embodiments, L is -L^{A4}-L^{A1}-L^{A4}-bb, and L^{A1} is —CH₂—, —CHMe-, or —CMe₂-. In some embodiments, L is -L^{A4}-L^{A}1-L^{A4}-_{bb}, and L^{A1} is —CH(OH)—.

In some embodiments, L is -L^{A4}-L^{A3}-L^{A4}-_{bb}. In some embodiments, L^{A3} is —C(═O).

In some embodiments, L is -L^{A4}-L^{A3}-L^{A4}-_{bb}, and L^{A3} is C(═O). In some embodiments, L is -L^{A4}-L^{A3}-L^{A4}-_{bb}, and L^{A3} is 0, NH, or N(C₁₋₃ alkyl). In some embodiments, L is -L^{A4}-L^{A3}-L^{A4}-_{bb}, and L is -L^{A4}-L^{A3}-L^{A4}-_{bb}, and L^{A3} is S(O)₂.

In some embodiments, L is -L^{A4}-L^{A1}-L^{A4}-L^{A3}-_{bb}, and L^{A3} is C(O).

In some embodiments, L is -L^{A4}-L^{A1}-L^{A4}-L^{A3}-_{bb}, and L^{A3} is O.

In some embodiments, L is -L^{A4}-L^{A1}-L^{A1}-L^{A4}-_{bb}, and one or both of L^{A1} is CH₂.

In some embodiments, L is -L^{A4}-L^{A1}-L^{A4}-L^{A3}-_{bb}. In some embodiments, L is -L^{A4}-L^{A}1-L^{A4}-O-bb.

In some embodiments, each L^{A4} is independently C3-10 cycloalkylene or 4-12 membered heterocyclylene, each of which is optionally substituted with 1-6 R^{a}. In some embodiments, each L^{A4} is independently 4-12 (e.g., 4-10) membered heterocyclylene optionally substituted with 1-3 R^{a}. In some embodiments, each R^{a} present on L^{A4} is independently selected from the group consisting of: —F; CN; C₁₋₃ alkoxy; OH; C₁₋₃ alkyl substituted with OH; and C₁₋₃ alkyl optionally substituted with 1-3 F. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, each L^{A4} is independently monocyclic 4-6 membered nitrogen-containing heterocyclylene optionally substituted with 1-3 R^{a}. In some embodiments, each L^{A4} contains 1-2 ring nitrogen atoms and no additional ring heteroatoms. In some embodiments, each R^{a} present on L^{A4} is independently selected from the group consisting of: —F; CN; C₁₋₃ alkoxy; OH; C₁₋₃ alkyl substituted with OH; and C₁₋₃ alkyl optionally substituted with 1-3 F. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, one L^{A4} is monocyclic 4-6 membered nitrogen-containing heterocyclylene optionally substituted with 1-3 R^{a}; and the other L^{A4} is bicyclic 6-12 (e.g., 6-10) membered nitrogen-containing heterocyclylene optionally substituted with 1-3 R^{a}. In some embodiments, each L^{A4} contains 1-2 ring nitrogen atoms and no additional ring heteroatoms. In some embodiments, each R^{a} present on L^{A4} is independently selected from the group consisting of: —F; CN; C₁₋₃ alkoxy; OH; C₁₋₃ alkyl substituted with OH; and C₁₋₃ alkyl optionally substituted with 1-3 F. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, one L^{A4} is monocyclic 4-6 membered nitrogen-containing heterocyclylene optionally substituted with 1-3 R^{a}; and the other L^{A4} is bicyclic spirocyclic 6-12 (e.g., 6-10) membered nitrogen-containing heterocyclylene optionally substituted with 1-3 R^{a}. In some embodiments, each L^{A4} contains 1-2 ring nitrogen atoms and no additional ring heteroatoms.

In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, L is selected from the group consisting of:
L^{A4}-L^{A1}-L^{A4}-_{bb};
L^{A4}-L^{A1}-L^{A1}-L^{A4}-_{bb};
L^{A4}-L^{A4}-_{bb};
L^{A4}-C(═O)-L^{A4}-_{bb}; and
L^{A4}-L^{A1}-L^{A4}-C(═O)-_{bb},
wherein bb represents the point of attachment to Ring C; and
L^{A1} is —CH₂—, —CHMe-, —CMe₂-, or —CH(OH)—;
each L^{A4} is independently a 4-12 membered nitrogen-containing heterocyclylene optionally substituted with 1-3 R^{a}, wherein:
each R^{a} present on L^{A4} is independently selected from the group consisting of: —F; CN; C₁ 3 alkoxy; OH; C₁₋₃ alkyl substituted with OH; and C₁₋₃ alkyl optionally substituted with 1-3 F.

In some embodiments, L is selected from the group consisting of:
L^{A4}-L^{A1}-L^{A4}-_{bb};
L^{A4}-L^{A1}-L^{A1}-L^{A4}-_{bb};
L^{A4}-L^{A4}-_{bb};
L^{A4}-C(═O)-L^{A4}-_{bb}; and
L^{A4}-L^{A1}-L^{A4}-C(═O)-_{bb},
wherein bb represents the point of attachment to Ring C; and
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments, L is L^{A4}-L^{A1}-L^{A4}-_{bb}, wherein:
bb represents the point of attachment to Ring C;
L^{A1} is —CH₂—, —CHMe-, —CMe₂-, or —CH(OH)—;
each L^{A4} is independently 4-12 membered heterocyclylene, each of which is optionally substituted with 1-3 R^{a}; and
each R^{a} present on L^{A4} is independently selected from the group consisting of: —F; CN; C₁ 3 alkoxy; OH; C₁₋₃ alkyl substituted with OH; and C₁₋₃ alkyl optionally substituted with 1-3 F.

In some embodiments, L is L^{A4}-L^{A1}-L^{A4}-_{bb}, wherein:
bb represents the point of attachment to Ring C;
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments, L is selected from the group consisting of:
L^{A4}-L^{A3}-_{bb};
L^{A3}-L^{A4}-_{bb};
L^{A4}-L^{A1}-_{bb} (e.g., -L^{A4}-CH₂—);
L^{A4}-L^{A1}-bb; and
L^{A4}-L^{A1}-L^{A3}-_{bb},
wherein bb represents the point of attachment to Ring C.

In some embodiments, L is selected from the group consisting of:
L^{A4}-L^{A3}-_{bb};
L^{A3}-L^{A4}-_{bb};
L^{A4}-L^{A1}-bb (e.g., -L^{A4}-CH₂—);
L^{A4}-L^{A1}-bb; and
L^{A4}-L^{A1}-L^{A3}-_{bb},
wherein bb represents the point of attachment to Ring C.

In some embodiments, L is selected from the group consisting of:
L^{A4}-L^{A3}-_{bb};
L^{A4}-L^{A1}-_{bb}; and
L^{A4}-L^{A1}-L^{A3}-_{bb},
wherein bb represents the point of attachment to Ring C.

In some embodiments, L is -L^{A4}-L^{A3}-_{bb}. In some embodiments, L^{A3} is —NH— or —N(C₁₋₃ alkyl)- (e.g., —NH—). In some embodiments, L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, L^{A4} is 4-12 membered heterocyclylene optionally substituted with 1-6 R^{a}. In some embodiments, L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, L^{A4} is 4-12 (e.g., 6-10) membered nitrogen-containing heterocyclylene optionally substituted with 1-3 R^{a}. In some embodiments, L^{A4} is bicyclic spirocyclic 6-12 (e.g., 6-10) membered nitrogen-containing heterocyclylene optionally substituted with 1-3 R^{a}. In some embodiments, L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, L is selected from the group consisting of the moieties delineated in Table L, wherein bb represents the point of attachment to Ring C.

TABLE L
Examples when L is —L^{A4}—L^{A1}—L^{A4}-_{bb}

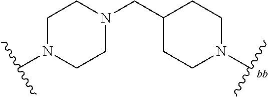 [SMILES: *N1CCC(CN2CCN(*)CC2)CC1]

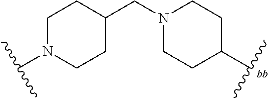 [SMILES: *C1CCN(CC2CCN(*)CC2)CC1]

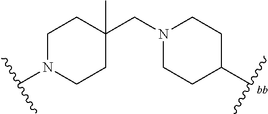 [SMILES: *C1CCN(CC2(C)CCN(*)CC2)CC1]

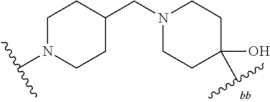 [SMILES: *N1CCC(CN2CCC(*)(O)CC2)CC1]

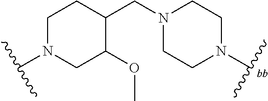 [SMILES: *N1CCN(CC2CCN(*)CC2OC)CC1]

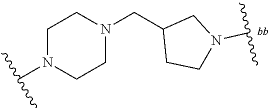 [SMILES: *N1CCN(CC2CCN(*)C2)CC1]

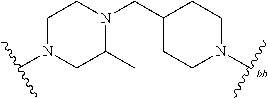 [SMILES: *N1CCC(CN2CCN(*)CC2C)CC1]

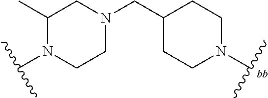 [SMILES: *N1CCC(CN2CCN(*)C(C)C2)CC1]

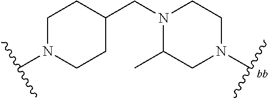 [SMILES: *N1CCC(CN2CCN(*)CC2C)CC1]

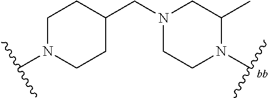 [SMILES: *N1CCC(CN2CCN(*)C(C)C2)CC1]

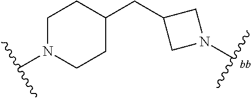 [SMILES: *N1CCC(CC2CN(*)C2)CC1]

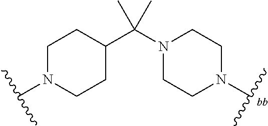 [SMILES: *N1CCC(C(C)(C)N2CCN(*)CC2)CC1]

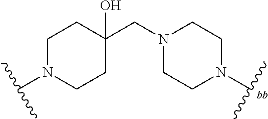 [SMILES: *N1CCN(CC2(O)CCN(*)CC2)CC1]

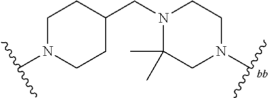 [SMILES: *N1CCC(CN2CCN(*)CC2(C)C)CC1]

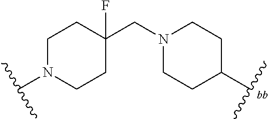 [SMILES: *C1CCN(CC2(F)CCN(*)CC2)CC1]

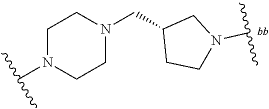 [SMILES: *N1CCN(C[C@H]2CCN(*)C2)CC1]

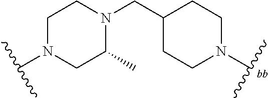 [SMILES: *N1CCC(CN2CCN(*)C[C@H]2C)CC1]

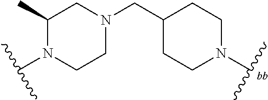 [SMILES: *N1CCC(CN2CCN(*)[C@@H](C)C2)CC1]

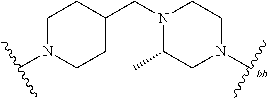 [SMILES: *N1CCC(CN2CCN(*)C[C@@H]2C)CC1]

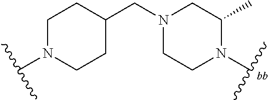 [SMILES: *N1CCC(CN2CCN(*)[C@@H](C)C2)CC1]

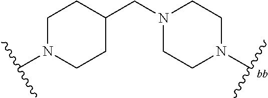 [SMILES: *N1CCC(CN2CCN(*)CC2)CC1]

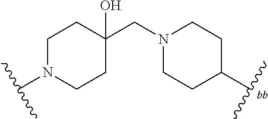 [SMILES: *C1CCN(CC2(O)CCN(*)CC2)CC1]

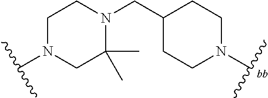 [SMILES: *N1CCC(CN2CCN(*)CC2(C)C)CC1]

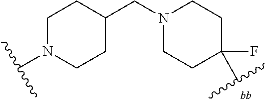 [SMILES: *N1CCC(CN2CCC(*)(F)CC2)CC1]

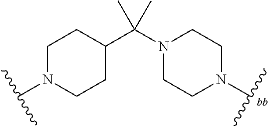 [SMILES: *N1CCC(C(C)(C)N2CCN(*)CC2)CC1]

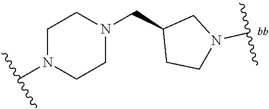 [SMILES: *N1CCN(C[C@@H]2CCN(*)C2)CC1]

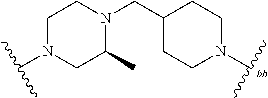 [SMILES: *N1CCC(CN2CCN(*)C[C@@H]2C)CC1]

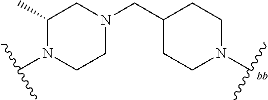 [SMILES: *N1CCC(CN2CCN(*)[C@H](C)C2)CC1]

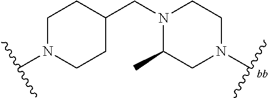 [SMILES: *N1CCC(CN2CCN(*)C[C@H]2C)CC1]

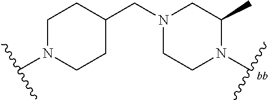 [SMILES: *N1CCC(CN2CCN(*)[C@H](C)C2)CC1]

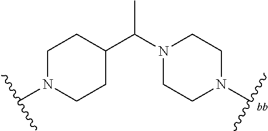 [SMILES: *N1CCC(C(C)N2CCN(*)CC2)CC1]

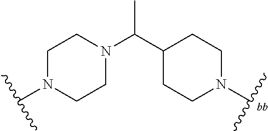 [SMILES: *N1CCC(C(C)N2CCN(*)CC2)CC1]

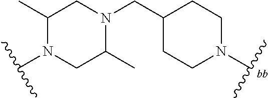 [SMILES: *N1CCC(CN2CC(C)N(*)CC2C)CC1]

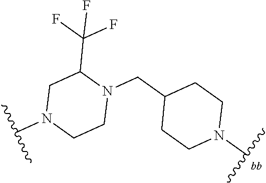 [SMILES: *N1CCC(CN2CCN(*)CC2C(F)(F)F)CC1]

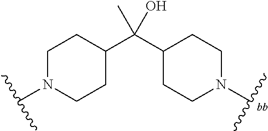 [SMILES: *N1CCC(C(C)(O)C2CCN(*)CC2)CC1]

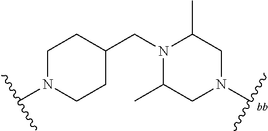 [SMILES: *N1CCC(CN2C(C)CN(*)CC2C)CC1]

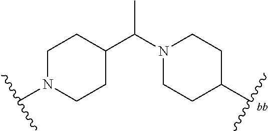 [SMILES: *C1CCN(C(C)C2CCN(*)CC2)CC1]

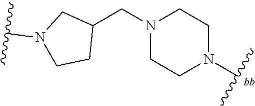 [SMILES: *N1CCN(CC2CCN(*)C2)CC1]

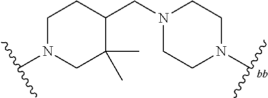 [SMILES: *N1CCN(CC2CCN(*)CC2(C)C)CC1]

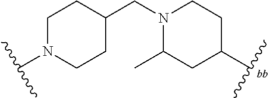 [SMILES: *C1CCN(CC2CCN(*)CC2)C(C)C1]

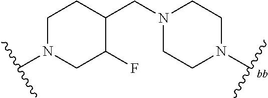 [SMILES: *N1CCN(CC2CCN(*)CC2F)CC1]

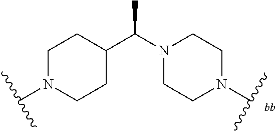 [SMILES: *N1CCC([C@@H](C)N2CCN(*)CC2)CC1]

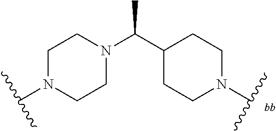 [SMILES: *N1CCC([C@H](C)N2CCN(*)CC2)CC1]

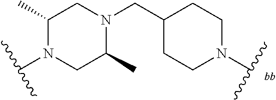 [SMILES: *N1CCC(CN2C[C@@H](C)N(*)C[C@@H]2C)CC1]

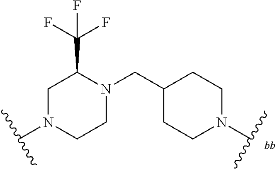 [SMILES: *N1CCC(CN2CCN(*)C[C@H]2C(F)(F)F)CC1]

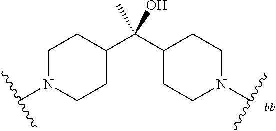

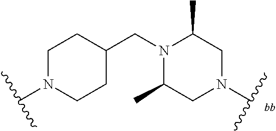 [SMILES: *N1CCC(CN2[C@H](C)CN(*)C[C@@H]2C)CC1]

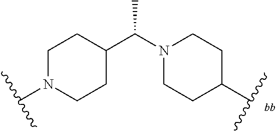 [SMILES: *C1CCN([C@@H](C)C2CCN(*)CC2)CC1]

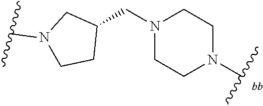 [SMILES: *N1CCN(C[C@@H]2CCN(*)C2)CC1]

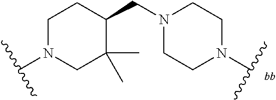 [SMILES: *N1CCN(C[C@@H]2CCN(*)CC2(C)C)CC1]

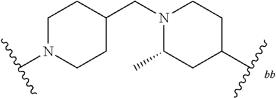 [SMILES: *C1CCN(CC2CCN(*)CC2)[C@@H](C)C1]

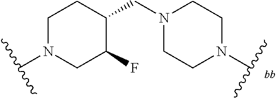 [SMILES: *N1CCN(C[C@H]2CCN(*)C[C@@H]2F)CC1]

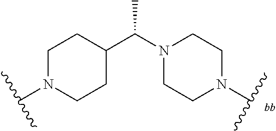 [SMILES: *N1CCC([C@H](C)N2CCN(*)CC2)CC1]

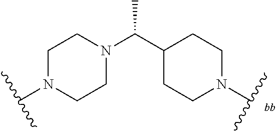 [SMILES: *N1CCC([C@@H](C)N2CCN(*)CC2)CC1]

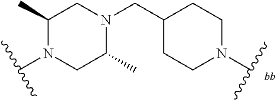 [SMILES: *N1CCC(CN2C[C@H](C)N(*)C[C@H]2C)CC1]

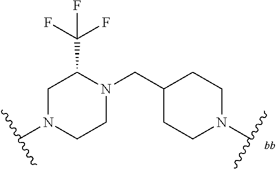 [SMILES: *N1CCC(CN2CCN(*)C[C@@H]2C(F)(F)F)CC1]

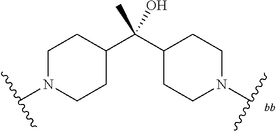

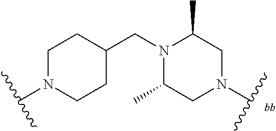 [SMILES: *N1CCC(CN2[C@@H](C)CN(*)C[C@@H]2C)CC1]

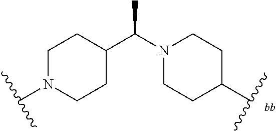 [SMILES: *C1CCN([C@H](C)C2CCN(*)CC2)CC1]

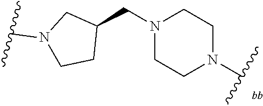 [SMILES: *N1CCN(C[C@H]2CCN(*)C2)CC1]

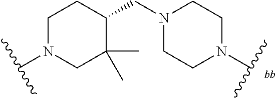 [SMILES: *N1CCN(C[C@H]2CCN(*)CC2(C)C)CC1]

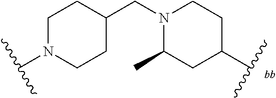 [SMILES: *C1CCN(CC2CCN(*)CC2)[C@H](C)C1]

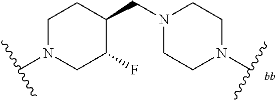 [SMILES: *N1CCN(C[C@@H]2CCN(*)C[C@H]2F)CC1]

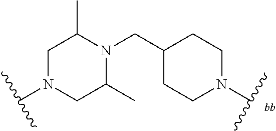 [SMILES: *N1CCC(CN2C(C)CN(*)CC2C)CC1]

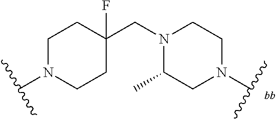 [SMILES: *N1CCC(F)(CN2CCN(*)C[C@@H]2C)CC1]

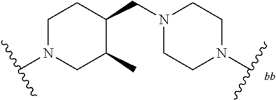 [SMILES: *N1CCN(C[C@@H]2CCN(*)C[C@@H]2C)CC1]

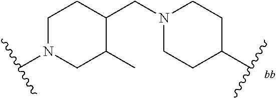 [SMILES: *C1CCN(CC2CCN(*)CC2C)CC1]

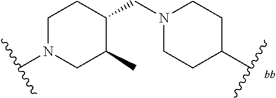 [SMILES: *C1CCN(C[C@H]2CCN(*)C[C@@H]2C)CC1]

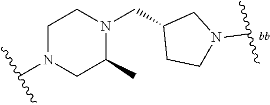 [SMILES: *N1CC[C@H](CN2CCN(*)C[C@@H]2C)C1]

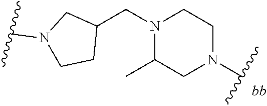 [SMILES: *N1CCC(CN2CCN(*)CC2C)C1]

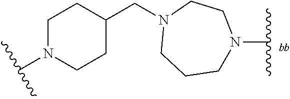 [SMILES: *N1CCC(CN2CCCN(*)CC2)CC1]

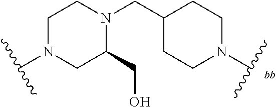 [SMILES: *N1CCC(CN2CCN(*)C[C@@H]2CO)CC1]

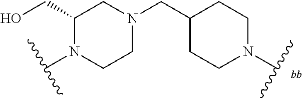 [SMILES: *N1CCC(CN2CCN(*)[C@H](CO)C2)CC1]

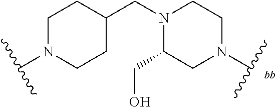 [SMILES: *N1CCC(CN2CCN(*)C[C@@H]2CO)CC1]

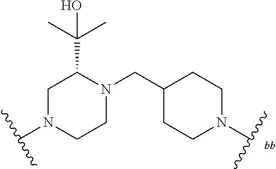 [SMILES: *N1CCC(CN2CCN(*)C[C@@H]2C(C)(C)O)CC1]

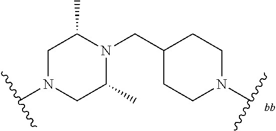 [SMILES: *N1CCC(CN2[C@H](C)CN(*)C[C@@H]2C)CC1]

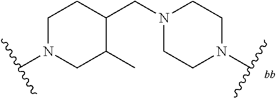 [SMILES: *N1CCN(CC2CCN(*)CC2C)CC1]

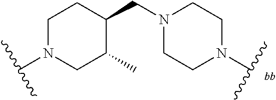 [SMILES: *N1CCN(C[C@@H]2CCN(*)C[C@H]2C)CC1]

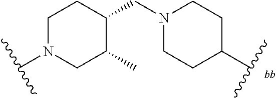 [SMILES: *C1CCN(C[C@H]2CCN(*)C[C@H]2C)CC1]

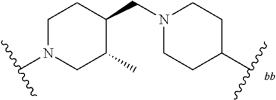 [SMILES: *C1CCN(C[C@@H]2CCN(*)C[C@H]2C)CC1]

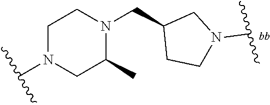 [SMILES: *N1CC[C@@H](CN2CCN(*)C[C@@H]2C)C1]

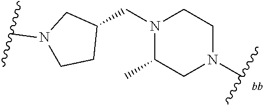 [SMILES: *N1CC[C@@H](CN2CCN(*)C[C@@H]2C)C1]

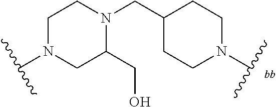 [SMILES: *N1CCC(CN2CCN(*)CC2CO)CC1]

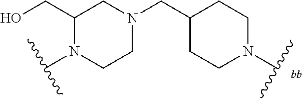 [SMILES: *N1CCC(CN2CCN(*)C(CO)C2)CC1]

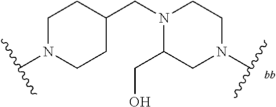 [SMILES: *N1CCC(CN2CCN(*)CC2CO)CC1]

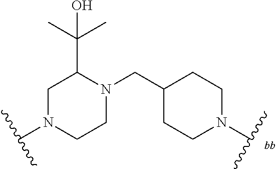 [SMILES: *N1CCC(CN2CCN(*)CC2C(C)(C)O)CC1]

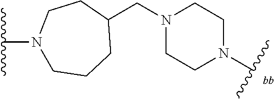 [SMILES: *N1CCCC(CN2CCN(*)CC2)CC1]

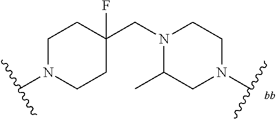 [SMILES: *N1CCC(F)(CN2CCN(*)CC2C)CC1]

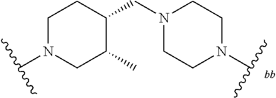 [SMILES: *N1CCN(C[C@H]2CCN(*)C[C@H]2C)CC1]

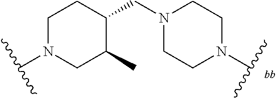 [SMILES: *N1CCN(C[C@H]2CCN(*)C[C@@H]2C)CC1]

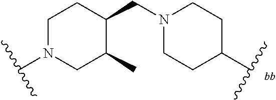 [SMILES: *C1CCN(C[C@@H]2CCN(*)C[C@@H]2C)CC1]

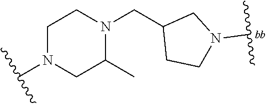 [SMILES: *N1CCC(CN2CCN(*)CC2C)C1]

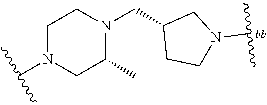 [SMILES: *N1CC[C@H](CN2CCN(*)C[C@H]2C)C1]

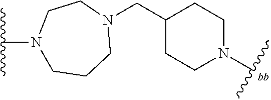 [SMILES: *N1CCC(CN2CCCN(*)CC2)CC1]

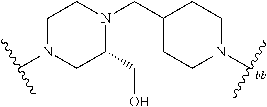 [SMILES: *N1CCC(CN2CCN(*)C[C@H]2CO)CC1]

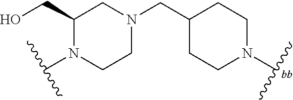 [SMILES: *N1CCC(CN2CCN(*)[C@@H](CO)C2)CC1]

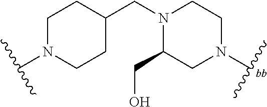 [SMILES: *N1CCC(CN2CCN(*)C[C@H]2CO)CC1]

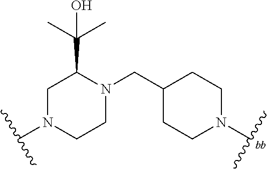 [SMILES: *N1CCC(CN2CCN(*)C[C@H]2C(C)(C)O)CC1]

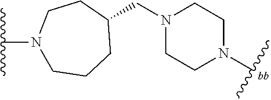 [SMILES: *N1CCC[C@@H](CN2CCN(*)CC2)CC1]

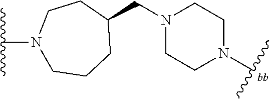 [SMILES: *N1CCC[C@H](CN2CCN(*)CC2)CC1]

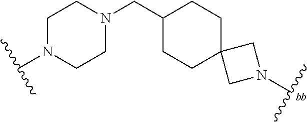 [SMILES: *N1CCN(CC2CCC3(CC2)CN(*)C3)CC1]

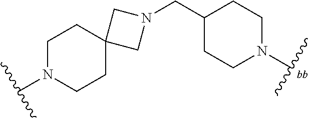 [SMILES: *N1CCC(CN2CC3(CCN(*)CC3)C2)CC1]

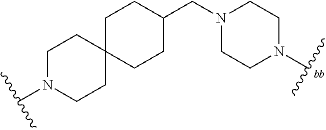 [SMILES: *N1CCN(CC2CCC3(CC2)CCN(*)CC3)CC1]

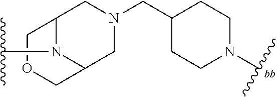 [SMILES: *N1CCC(CN2CC3COCC(C2)N3*)CC1]

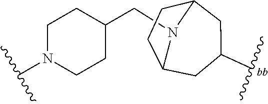 [SMILES: *C1CC2CCC(C1)N2CC1CCN(*)CC1]

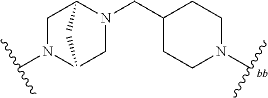

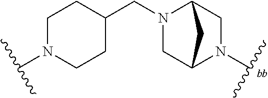 [SMILES: *N1CCC(CN2C[C@@H]3C[C@H]2CN3*)CC1]

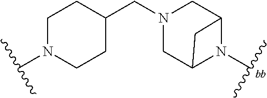 [SMILES: *N1CCC(CN2CC3CC(C2)N3*)CC1]

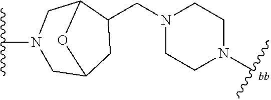 [SMILES: *N1CCN(CC2CC3CN(*)CC2O3)CC1]

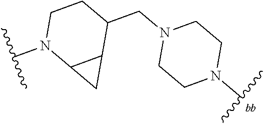 [SMILES: *N1CCN(CC2CCN(*)C3CC23)CC1]

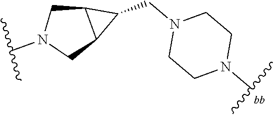 [SMILES: *N1CCN(C[C@H]2[C@@H]3CN(*)C[C@@H]32)CC1]

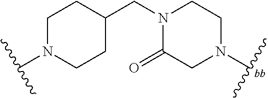 [SMILES: *N1CCC(CN2CCN(*)CC2=O)CC1]

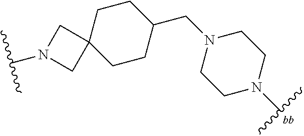 [SMILES: *N1CCN(CC2CCC3(CC2)CN(*)C3)CC1]

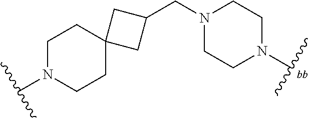 [SMILES: *N1CCN(CC2CC3(CCN(*)CC3)C2)CC1]

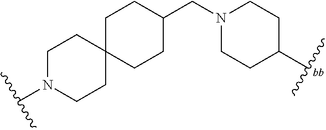 [SMILES: *C1CCN(CC2CCC3(CC2)CCN(*)CC3)CC1]

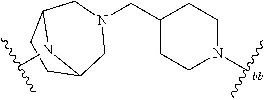 [SMILES: *N1CCC(CN2CC3CCC(C2)N3*)CC1]

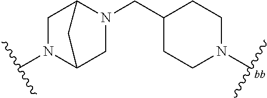 [SMILES: *N1CCC(CN2CC3CC2CN3*)CC1]

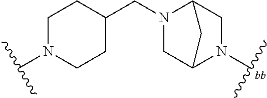 [SMILES: *N1CCC(CN2CC3CC2CN3*)CC1]

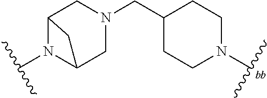 [SMILES: *N1CCC(CN2CC3CC(C2)N3*)CC1]

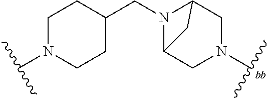 [SMILES: *N1CCC(CN2C3CC2CN(*)C3)CC1]

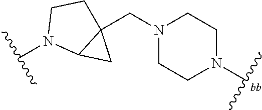 [SMILES: *N1CCN(CC23CCN(*)C2C3)CC1]

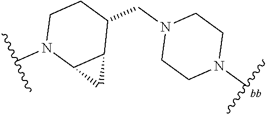 [SMILES: *N1CCN(C[C@H]2CCN(*)[C@H]3C[C@@H]23)CC1]

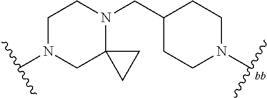 [SMILES: *N1CCC(CN2CCN(*)CC23CC3)CC1]

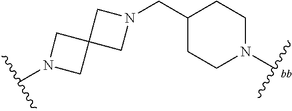 [SMILES: *N1CCC(CN2CC3(CN(*)C3)C2)CC1]

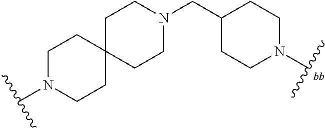 [SMILES: *N1CCC(CN2CCC3(CCN(*)CC3)CC2)CC1]

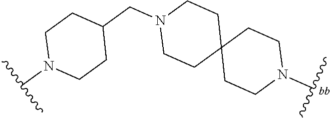 [SMILES: *N1CCC(CN2CCC3(CCN(*)CC3)CC2)CC1]

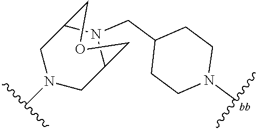 [SMILES: *N1CCC(CN2C3COCC2CN(*)C3)CC1]

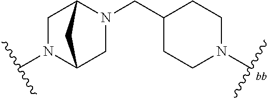 [SMILES: *N1CCC(CN2C[C@H]3C[C@@H]2CN3*)CC1]

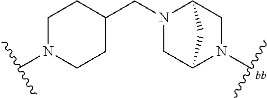 [SMILES: *N1CCC(CN2C[C@H]3C[C@@H]2CN3*)CC1]

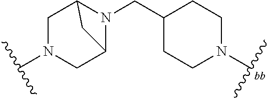 [SMILES: *N1CCC(CN2C3CC2CN(*)C3)CC1]

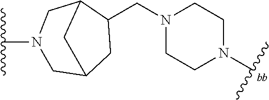 [SMILES: *N1CCN(CC2CC3CC2CN(*)C3)CC1]

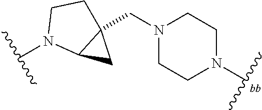 [SMILES: *N1CCN(C[C@@]23CCN(*)[C@@H]2C3)CC1]

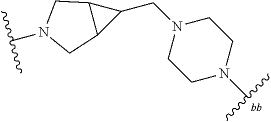 [SMILES: *N1CCN(CC2C3CN(*)CC32)CC1]

Examples when L is —L^{A4}—L^{A4}-_{bb}

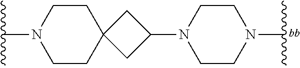 [SMILES: *N1CCN(C2CC3(CCN(*)CC3)C2)CC1]

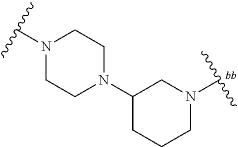 [SMILES: *N1CCN(C2CCCN(*)C2)CC1]

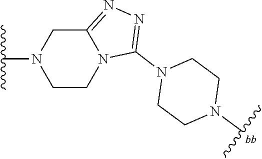 [SMILES: *N1CCN(c2nnc3n2CCN(*)C3)CC1]

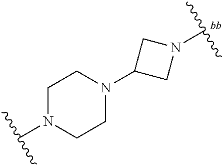 [SMILES: *N1CCN(C2CN(*)C2)CC1]

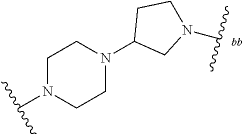 [SMILES: *N1CCN(C2CCN(*)C2)CC1]

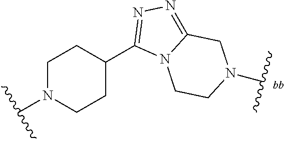 [SMILES: *N1CCC(c2nnc3n2CCN(*)C3)CC1]

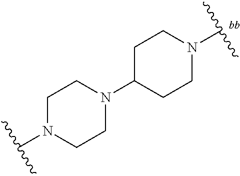 [SMILES: *N1CCC(N2CCN(*)CC2)CC1]

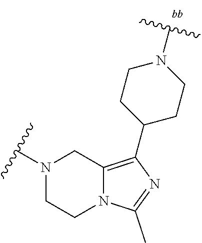 [SMILES: *N1CCC(c2nc(C)n3c2CN(*)CC3)CC1]

Examples when L is —L^{A4}—L^{A1}—L^{A1}—L^{A4}-_{bb}

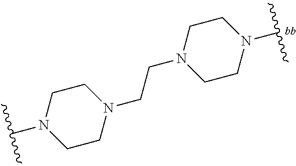 [SMILES: *N1CCN(CCN2CCN(*)CC2)CC1]

Examples when L is —L^{A4}—L^{A3}—L^{A4}-_{bb}

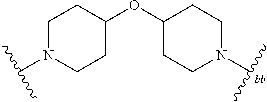 [SMILES: *N1CCC(OC2CCN(*)CC2)CC1]

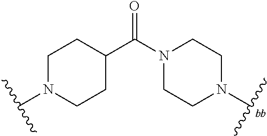 [SMILES: *N1CCC(C(=O)N2CCN(*)CC2)CC1]

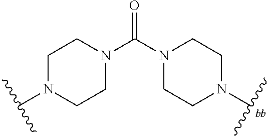 [SMILES: *N1CCN(C(=O)N2CCN(*)CC2)CC1]

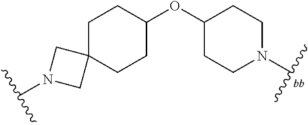 [SMILES: *N1CCC(OC2CCC3(CC2)CN(*)C3)CC1]

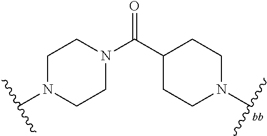 [SMILES: *N1CCC(C(=O)N2CCN(*)CC2)CC1]

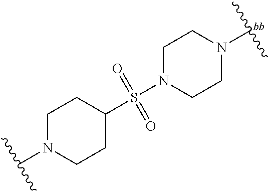 [SMILES: *N1CCC(S(=O)(=O)N2CCN(*)CC2)CC1]

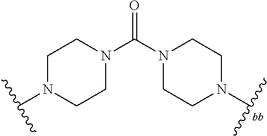 [SMILES: *N1CCN(C(=O)N2CCN(*)CC2)CC1]

Examples when L is —L^{A4}—L^{A1}—L^{A4}—L^{A3}-_{bb}

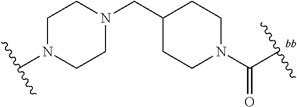 [SMILES: *C(=O)N1CCC(CN2CCN(*)CC2)CC1]

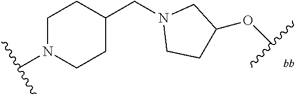 [SMILES: *OC1CCN(CC2CCN(*)CC2)C1]

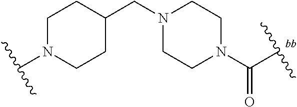 [SMILES: *C(=O)N1CCN(CC2CCN(*)CC2)CC1]

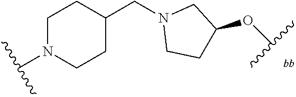 [SMILES: *O[C@H]1CCN(CC2CCN(*)CC2)C1]

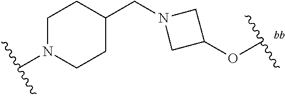 [SMILES: *OC1CN(CC2CCN(*)CC2)C1]

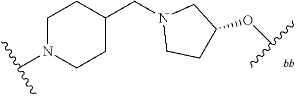 [SMILES: *O[C@@H]1CCN(CC2CCN(*)CC2)C1]

Examples when L is —L^{A4}—L^{A3}-_{bb}

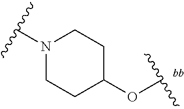 [SMILES: *OC1CCN(*)CC1]

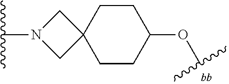 [SMILES: *OC1CCC2(CC1)CN(*)C2]

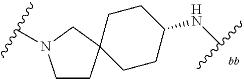

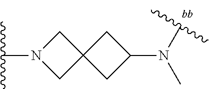 [SMILES: *N1CC2(CC(N(*)C)C2)C1]

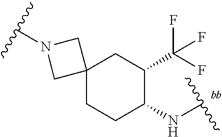 [SMILES: *N[C@@H]1CCC2(C[C@@H]1C(F)(F)F)CN(*)C2]

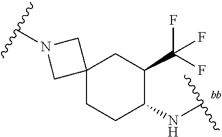 [SMILES: *N[C@@H]1CCC2(C[C@H]1C(F)(F)F)CN(*)C2]

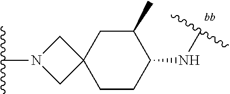 [SMILES: *N[C@@H]1CCC2(C[C@H]1C)CN(*)C2]

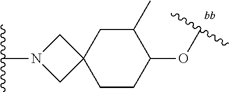 [SMILES: *OC1CCC2(CC1C)CN(*)C2]

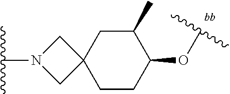 [SMILES: *O[C@H]1CCC2(C[C@H]1C)CN(*)C2]

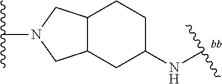 [SMILES: *NC1CCC2CN(*)CC2C1]

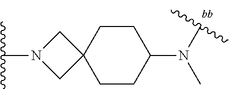 [SMILES: *N1CC2(CCC(N(*)C)CC2)C1]

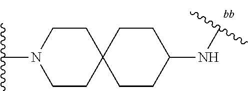 [SMILES: *NC1CCC2(CC1)CCN(*)CC2]

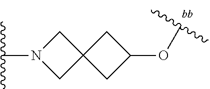 [SMILES: *OC1CC2(C1)CN(*)C2]

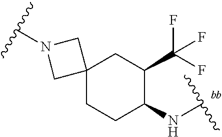 [SMILES: *N[C@H]1CCC2(C[C@H]1C(F)(F)F)CN(*)C2]

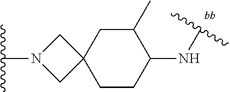 [SMILES: *NC1CCC2(CC1C)CN(*)C2]

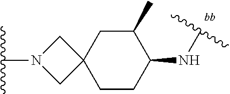 [SMILES: *N[C@H]1CCC2(C[C@H]1C)CN(*)C2]

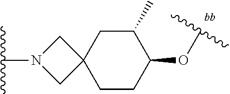 [SMILES: *O[C@H]1CCC2(C[C@@H]1C)CN(*)C2]

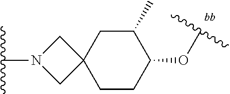 [SMILES: *O[C@@H]1CCC2(C[C@@H]1C)CN(*)C2]

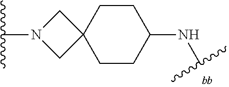 [SMILES: *NC1CCC2(CC1)CN(*)C2]

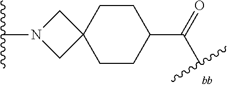 [SMILES: *C(=O)C1CCC2(CC1)CN(*)C2]

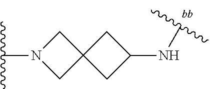 [SMILES: *NC1CC2(C1)CN(*)C2]

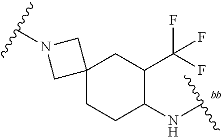 [SMILES: *NC1CCC2(CC1C(F)(F)F)CN(*)C2]

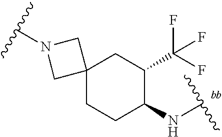 [SMILES: *N[C@H]1CCC2(C[C@@H]1C(F)(F)F)CN(*)C2]

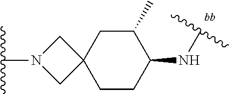 [SMILES: *N[C@H]1CCC2(C[C@@H]1C)CN(*)C2]

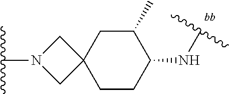 [SMILES: *N[C@@H]1CCC2(C[C@@H]1C)CN(*)C2]

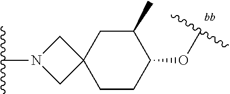 [SMILES: *O[C@@H]1CCC2(C[C@H]1C)CN(*)C2]

Examples when L is —L^{A4}—L^{A4}—L^{A3}—L^{A4}-_{bb}
or
—L^{A4}—L^{A3}—L^{A4}—L^{A4}-_{bb}

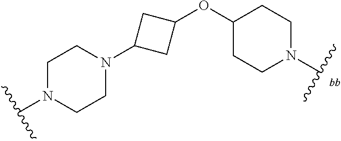 [SMILES: *N1CCC(OC2CC(N3CCN(*)CC3)C2)CC1]

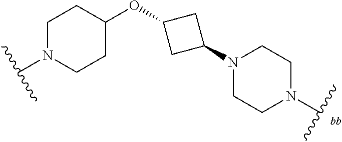 [SMILES: *N1CCC(O[C@H]2C[C@H](N3CCN(*)CC3)C2)CC1]

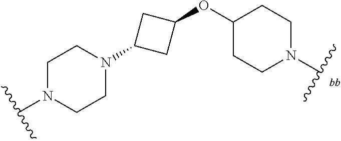 [SMILES: *N1CCC(O[C@H]2C[C@H](N3CCN(*)CC3)C2)CC1]

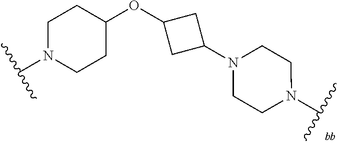 [SMILES: *N1CCC(OC2CC(N3CCN(*)CC3)C2)CC1]

Examples when L is —L^{A4}—

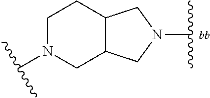 [SMILES: *N1CCC2CN(*)CC2C1]

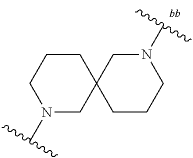 [SMILES: *N1CCCC2(CCCN(*)C2)C1]

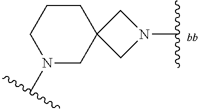 [SMILES: *N1CCCC2(C1)CN(*)C2]

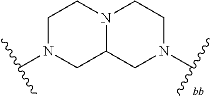 [SMILES: *N1CCN2CCN(*)CC2C1]

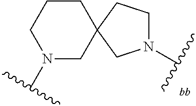 [SMILES: *N1CCCC2(CCN(*)C2)C1]

Examples when L is —L^{A4}—L^{A1}—L^{A4}—L^{A1}-_{bb}

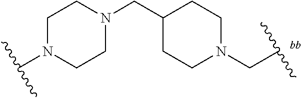 [SMILES: *CN1CCC(CN2CCN(*)CC2)CC1]

Examples when L is —L^{A4}—L^{A1}_{bb}

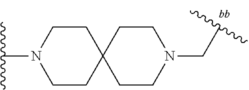 [SMILES: *CN1CCC2(CCN(*)CC2)CC1]

Examples when L is —L^{A3}—L^{A4}-_{bb}

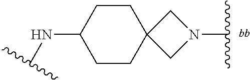 [SMILES: *NC1CCC2(CC1)CN(*)C2]

Examples when L is —L^{A4}—L^{A1}—L^{A3}-_{bb}
or
—L^{A3}—L^{A1}—L^{A4}—L^{A3}-_{bb}

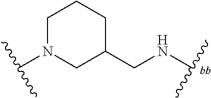 [SMILES: *NCC1CCCN(*)C1]

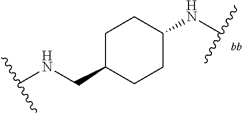 [SMILES: *NC[C@H]1CC[C@H](N*)CC1]

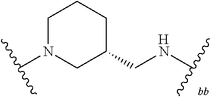

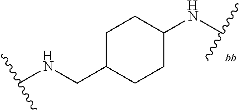

In some embodiments, L is selected from the group consisting of the moieties delineated in Table L1-a:

TABLE L1-a

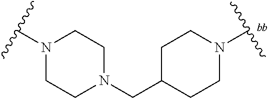

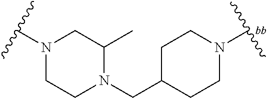

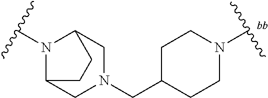

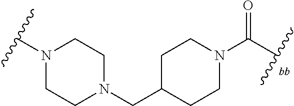

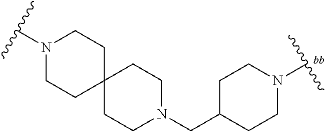

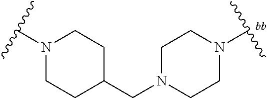

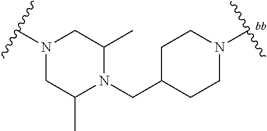

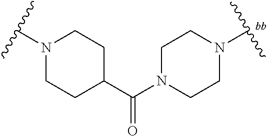

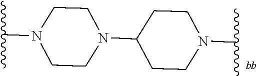

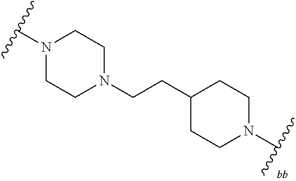

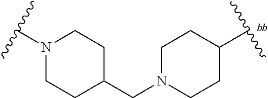

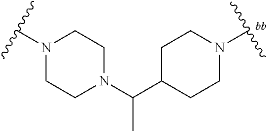

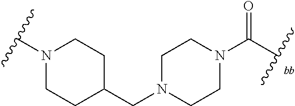

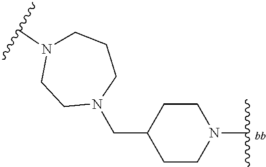

wherein bb represents the point of attachment to Ring C.

In some embodiments, L is selected from the group consisting of the moieties delineated in Table L2-a:

TABLE L2-a

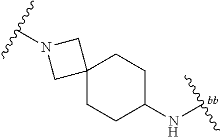

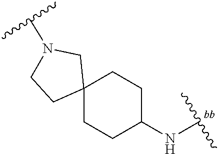

wherein bb represents the point of attachment to Ring C.

In some embodiments, L is -L^{A4}-L^{A4}-L^{A3}-L^{A4}-_{bb} or -L^{A4}-L^{A3}-L^{A4}-L^{A4}-_{bb}, wherein bb represents the point of attachment to Ring C.

In some embodiments, L is -L^{A4}-(C₃₋₆ cycloalkylene)-O-L^{A4}-_{bb} or -L^{A4}-O—(C₃₋₆ cycloalkylene)-L^{A4}-_{bb}, wherein the C₃₋₆ cycloalkylene is optionally substituted with 1-2 R^{a}; and each L^{A4} is independently a 4-12 membered heterocyclylene (e.g., 5-6 membered (e.g., 6-membered)) optionally substituted with 1-2 R^{a}.

In some embodiments, L is

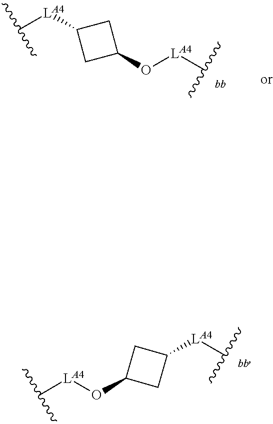

wherein each L^{A4} is a monocyclic 4-6 membered heterocyclylene optionally substituted with 1-2 R^{a}.

In some embodiments, L is L^{A4}, wherein the L^{A4} is 6-12 (e.g., 8-12 membered bicyclic heterocyclylene (e.g., 9-11 membered bicyclic heterocyclylene) membered bicyclic or tricyclic heterocyclylene optionally substituted with 1-2 R^{a}.

In some embodiments, the compounds of Formula (I) are compounds of Formula (I-aa):

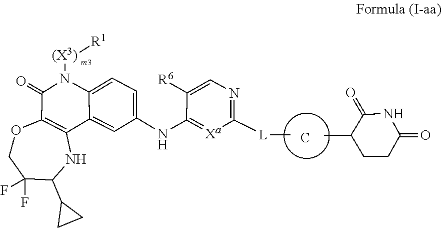

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

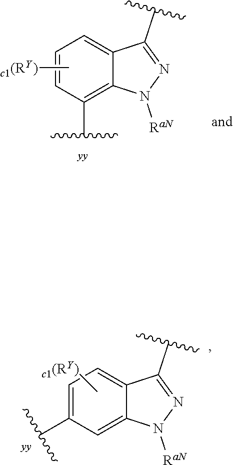

wherein: c1 is 0 or 1, R^{Y} is selected from the group consisting of halo (e.g., —F) and C1-3 alkyl optionally substituted with 1-3 F, and R^{aN} is C₁₋₃ alkyl;
L is selected from the group consisting of:
   L^{A4}-L^{A1}-L^{A4}-_{bb};
   L^{A4}-L^{A1}-L^{A1}-L^{A4}-_{bb};
   L^{A4}-L^{A4}-_{bb};
   L^{A4}-C(═O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms.

In some embodiments of Formula (I-aa), one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

In some embodiments of Formula (I-aa), L is -L^{A4}-L^{A1}-L^{A4}-_{bb}; and L^{A1} is CH₂ or CHMe.

In some embodiments of Formula (I-aa), L is selected from the group consisting of the moieties depicted in Table L-I-a.

TABLE L-I-a

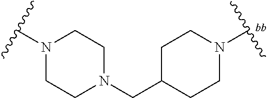 [SMILES: *N1CCC(CN2CCN(*)CC2)CC1]

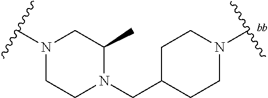 [SMILES: *N1CCC(CN2CCN(*)C[C@H]2C)CC1]

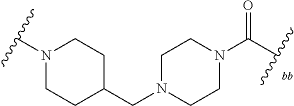 [SMILES: *C(=O)N1CCN(CC2CCN(*)CC2)CC1]

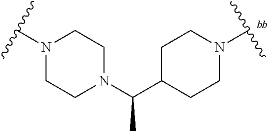 [SMILES: *N1CCC([C@@H](C)N2CCN(*)CC2)CC1]

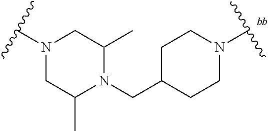 [SMILES: *N1CCC(CN2C(C)CN(*)CC2C)CC1]

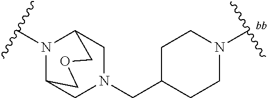 [SMILES: *N1CCC(CN2CC3COCC(C2)N3*)CC1]

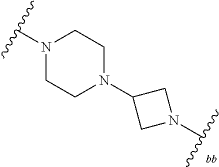 [SMILES: *N1CCN(C2CN(*)C2)CC1]

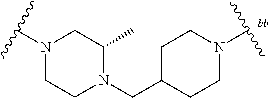 [SMILES: *N1CCC(CN2CCN(*)C[C@@H]2C)CC1]

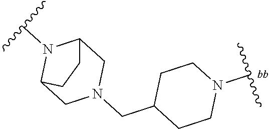 [SMILES: *N1CCC(CN2CC3CCC(C2)N3*)CC1]

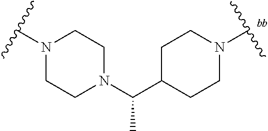 [SMILES: *N1CCC([C@H](C)N2CCN(*)CC2)CC1]

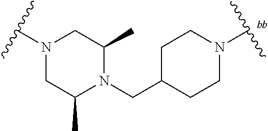 [SMILES: *N1CCC(CN2[C@H](C)CN(*)C[C@@H]2C)CC1]

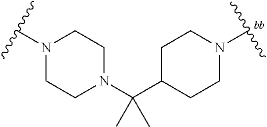 [SMILES: *N1CCC(C(C)(C)N2CCN(*)CC2)CC1]

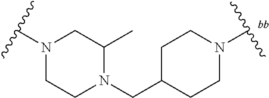 [SMILES: *N1CCC(CN2CCN(*)CC2C)CC1]

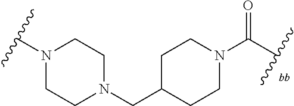 [SMILES: *C(=O)N1CCC(CN2CCN(*)CC2)CC1]

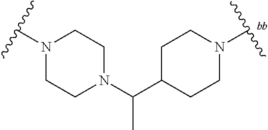 [SMILES: *N1CCC(C(C)N2CCN(*)CC2)CC1]

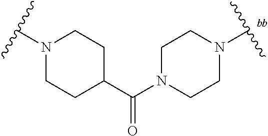 [SMILES: *N1CCC(C(=O)N2CCN(*)CC2)CC1]

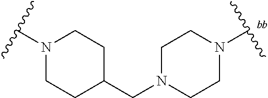 [SMILES: *N1CCC(CN2CCN(*)CC2)CC1]

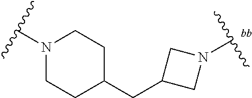 [SMILES: *N1CCC(CC2CN(*)C2)CC1]

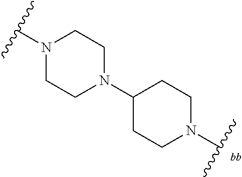 [SMILES: *N1CCC(N2CCN(*)CC2)CC1]

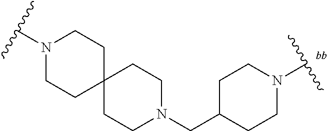 [SMILES: *N1CCC(CN2CCC3(CCN(*)CC3)CC2)CC1]

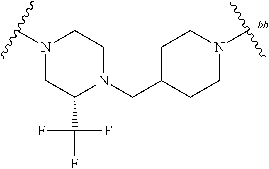 [SMILES: *N1CCC(CN2CCN(*)C[C@H]2C(F)(F)F)CC1]

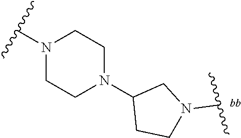 [SMILES: *N1CCN(C2CCN(*)C2)CC1]

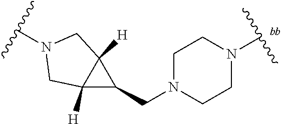 [SMILES: *N1CCN(C[C@H]2[C@@H]3CN(*)C[C@@H]32)CC1]

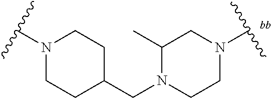 [SMILES: *N1CCC(CN2CCN(*)CC2C)CC1]

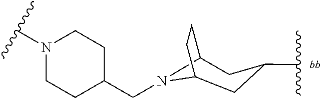 [SMILES: *C1CC2CCC(C1)N2CC1CCN(*)CC1]

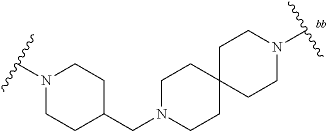 [SMILES: *N1CCC(CN2CCC3(CCN(*)CC3)CC2)CC1]

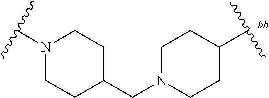 [SMILES: *C1CCN(CC2CCN(*)CC2)CC1]

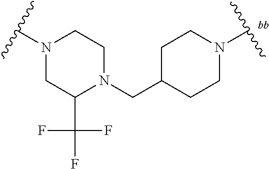 [SMILES: *N1CCC(CN2CCN(*)CC2C(F)(F)F)CC1]

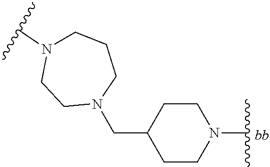 [SMILES: *N1CCC(CN2CCCN(*)CC2)CC1]

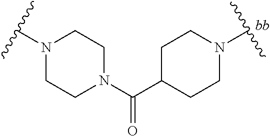 [SMILES: *N1CCC(C(=O)N2CCN(*)CC2)CC1]

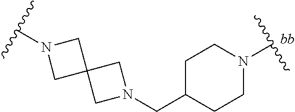 [SMILES: *N1CCC(CN2CC3(CN(*)C3)C2)CC1]

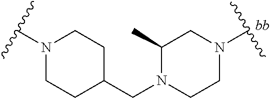 [SMILES: *N1CCC(CN2CCN(*)C[C@@H]2C)CC1]

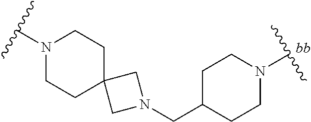 [SMILES: *N1CCC(CN2CC3(CCN(*)CC3)C2)CC1]

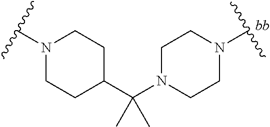 [SMILES: *N1CCC(C(C)(C)N2CCN(*)CC2)CC1]

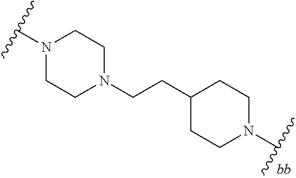 [SMILES: *N1CCC(CCN2CCN(*)CC2)CC1]

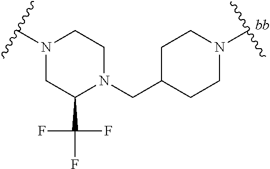 [SMILES: *N1CCC(CN2CCN(*)C[C@@H]2C(F)(F)F)CC1]

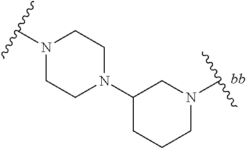 [SMILES: *N1CCN(C2CCCN(*)C2)CC1]

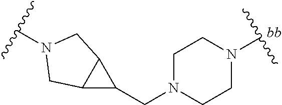 [SMILES: *N1CCN(CC2C3CN(*)CC32)CC1]

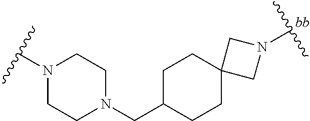 [SMILES: *N1CCN(CC2CCC3(CC2)CN(*)C3)CC1]

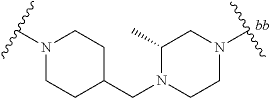 [SMILES: *N1CCC(CN2CCN(*)C[C@H]2C)CC1]

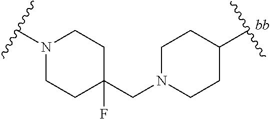 [SMILES: *C1CCN(CC2(F)CCN(*)CC2)CC1]

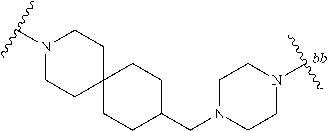 [SMILES: *N1CCN(CC2CCC3(CC2)CCN(*)CC3)CC1]

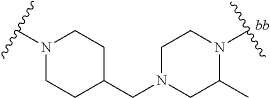 [SMILES: *N1CCC(CN2CCN(*)C(C)C2)CC1]

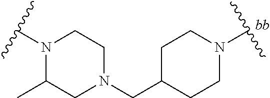 [SMILES: *N1CCC(CN2CCN(*)C(C)C2)CC1]

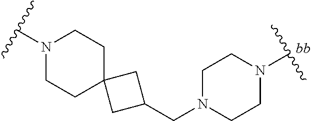 [SMILES: *N1CCN(CC2CC3(CCN(*)CC3)C2)CC1]

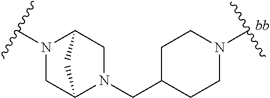 [SMILES: *N1CCC(CN2C[C@H]3C[C@@H]2CN3*)CC1]

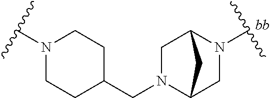 [SMILES: *N1CCC(CN2C[C@H]3C[C@@H]2CN3*)CC1]

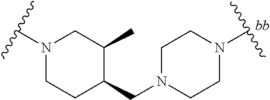 [SMILES: *N1CCN(C[C@H]2CCN(*)C[C@H]2C)CC1]

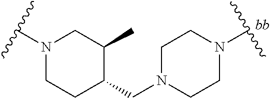 [SMILES: *N1CCN(C[C@@H]2CCN(*)C[C@H]2C)CC1]

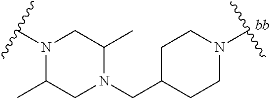 [SMILES: *N1CCC(CN2CC(C)N(*)CC2C)CC1]

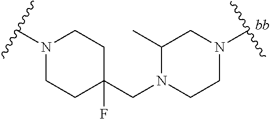 [SMILES: *N1CCC(F)(CN2CCN(*)CC2C)CC1]

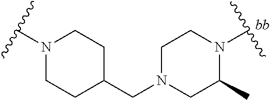 [SMILES: *N1CCC(CN2CCN(*)[C@@H](C)C2)CC1]

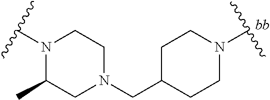 [SMILES: *N1CCC(CN2CCN(*)[C@H](C)C2)CC1]

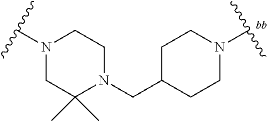 [SMILES: *N1CCC(CN2CCN(*)CC2(C)C)CC1]

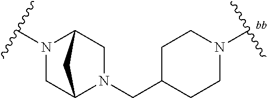 [SMILES: *N1CCC(CN2C[C@@H]3C[C@H]2CN3*)CC1]

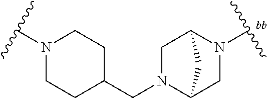 [SMILES: *N1CCC(CN2C[C@@H]3C[C@H]2CN3*)CC1]

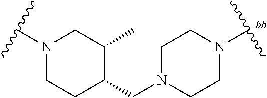 [SMILES: *N1CCN(C[C@@H]2CCN(*)C[C@@H]2C)CC1]

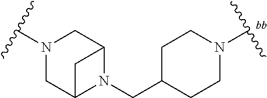 [SMILES: *N1CCC(CN2C3CC2CN(*)C3)CC1]

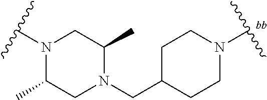 [SMILES: *N1CCC(CN2C[C@H](C)N(*)C[C@H]2C)CC1]

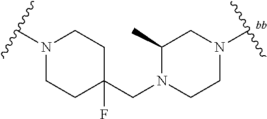 [SMILES: *N1CCC(F)(CN2CCN(*)C[C@@H]2C)CC1]

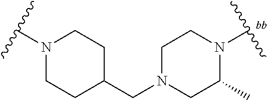 [SMILES: *N1CCC(CN2CCN(*)[C@H](C)C2)CC1]

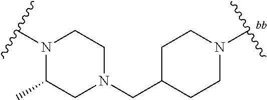 [SMILES: *N1CCC(CN2CCN(*)[C@@H](C)C2)CC1]

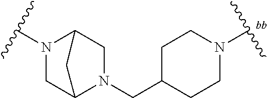 [SMILES: *N1CCC(CN2CC3CC2CN3*)CC1]

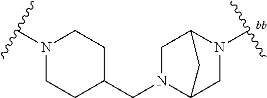 [SMILES: *N1CCC(CN2CC3CC2CN3*)CC1]

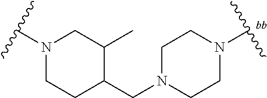 [SMILES: *N1CCN(CC2CCN(*)CC2C)CC1]

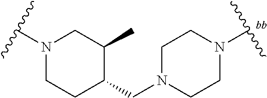 [SMILES: *N1CCN(C[C@@H]2CCN(*)C[C@H]2C)CC1]

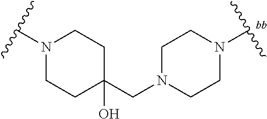 [SMILES: *N1CCN(CC2(O)CCN(*)CC2)CC1]

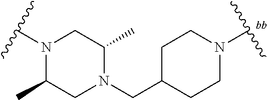 [SMILES: *N1CCC(CN2C[C@@H](C)N(*)C[C@@H]2C)CC1]

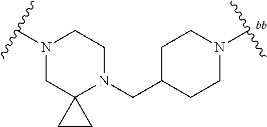 [SMILES: *N1CCC(CN2CCN(*)CC23CC3)CC1]

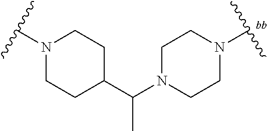 [SMILES: *N1CCC(C(C)N2CCN(*)CC2)CC1]

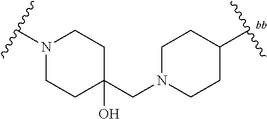 [SMILES: *C1CCN(CC2(O)CCN(*)CC2)CC1]

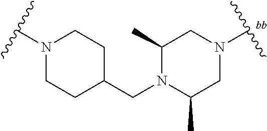 [SMILES: *N1CCC(CN2[C@H](C)CN(*)C[C@@H]2C)CC1]

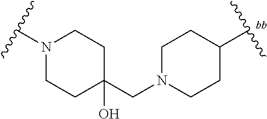 [SMILES: *C1CCN(CC2(O)CCN(*)CC2)CC1]

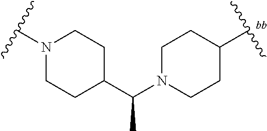 [SMILES: *C1CCN([C@@H](C)C2CCN(*)CC2)CC1]

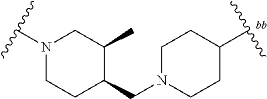 [SMILES: *C1CCN(C[C@H]2CCN(*)C[C@H]2C)CC1]

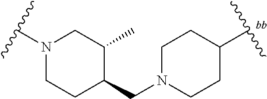 [SMILES: *C1CCN(C[C@H]2CCN(*)C[C@@H]2C)CC1]

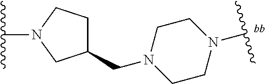 [SMILES: *N1CCN(C[C@@H]2CCN(*)C2)CC1]

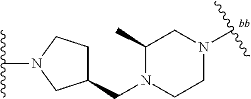 [SMILES: *N1CC[C@@H](CN2CCN(*)C[C@@H]2C)C1]

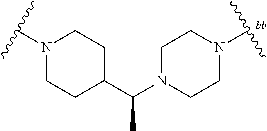 [SMILES: *N1CCC([C@H](C)N2CCN(*)CC2)CC1]

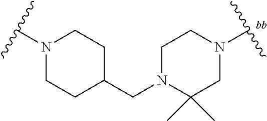 [SMILES: *N1CCC(CN2CCN(*)CC2(C)C)CC1]

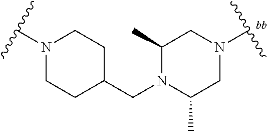 [SMILES: *N1CCC(CN2[C@@H](C)CN(*)C[C@@H]2C)CC1]

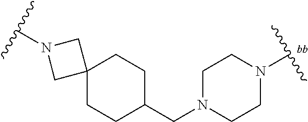 [SMILES: *N1CCN(CC2CCC3(CC2)CN(*)C3)CC1]

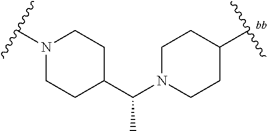 [SMILES: *C1CCN([C@H](C)C2CCN(*)CC2)CC1]

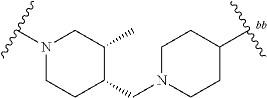 [SMILES: *C1CCN(C[C@@H]2CCN(*)C[C@@H]2C)CC1]

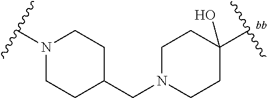 [SMILES: *N1CCC(CN2CCC(*)(O)CC2)CC1]

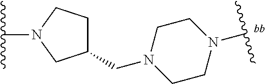 [SMILES: *N1CCN(C[C@H]2CCN(*)C2)CC1]

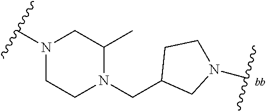 [SMILES: *N1CCC(CN2CCN(*)CC2C)C1]

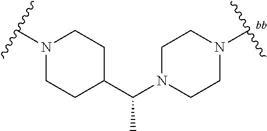 [SMILES: *N1CCC([C@@H](C)N2CCN(*)CC2)CC1]

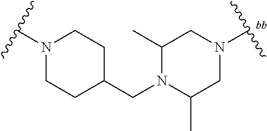 [SMILES: *N1CCC(CN2C(C)CN(*)CC2C)CC1]

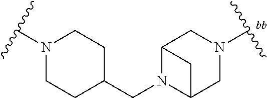 [SMILES: *N1CCC(CN2C3CC2CN(*)C3)CC1]

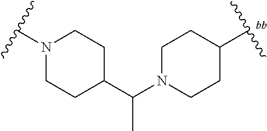 [SMILES: *C1CCN(C(C)C2CCN(*)CC2)CC1]

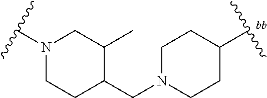 [SMILES: *C1CCN(CC2CCN(*)CC2C)CC1]

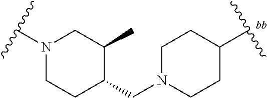 [SMILES: *C1CCN(C[C@@H]2CCN(*)C[C@H]2C)CC1]

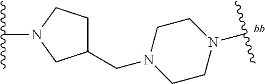 [SMILES: *N1CCN(CC2CCN(*)C2)CC1]

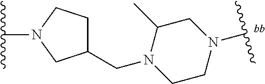 [SMILES: *N1CCC(CN2CCN(*)CC2C)C1]

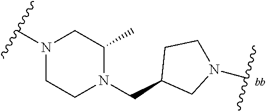 [SMILES: *N1CC[C@H](CN2CCN(*)C[C@@H]2C)C1]

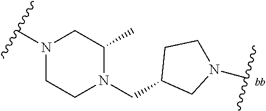 [SMILES: *N1CC[C@@H](CN2CCN(*)C[C@@H]2C)C1]

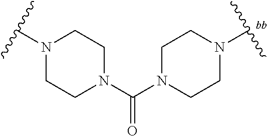 [SMILES: *N1CCN(C(=O)N2CCN(*)CC2)CC1]

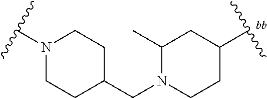 [SMILES: *C1CCN(CC2CCN(*)CC2)C(C)C1]

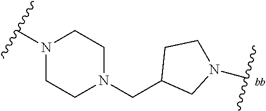 [SMILES: *N1CCN(CC2CCN(*)C2)CC1]

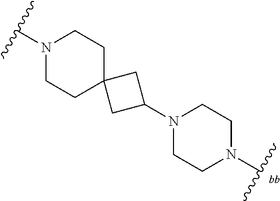 [SMILES: *N1CCN(C2CC3(CCN(*)CC3)C2)CC1]

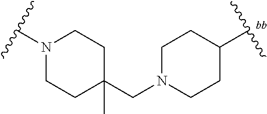 [SMILES: *C1CCN(CC2(C)CCN(*)CC2)CC1]

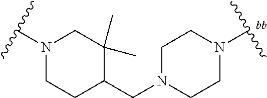 [SMILES: *N1CCN(CC2CCN(*)CC2(C)C)CC1]

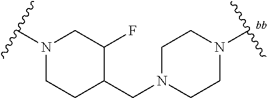 [SMILES: *N1CCN(CC2CCN(*)CC2F)CC1]

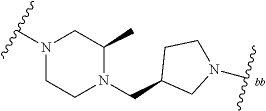 [SMILES: *N1CC[C@H](CN2CCN(*)C[C@H]2C)C1]

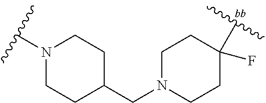 [SMILES: *N1CCC(CN2CCC(*)(F)CC2)CC1]

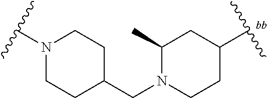 [SMILES: *C1CCN(CC2CCN(*)CC2)[C@@H](C)C1]

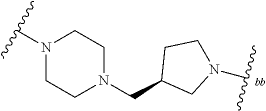 [SMILES: *N1CCN(C[C@H]2CCN(*)C2)CC1]

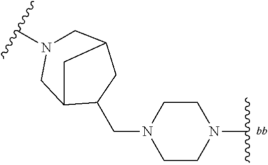 [SMILES: *N1CCN(CC2CC3CC2CN(*)C3)CC1]

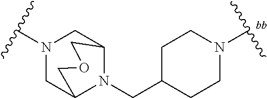 [SMILES: *N1CCC(CN2C3COCC2CN(*)C3)CC1]

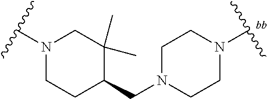 [SMILES: *N1CCN(C[C@H]2CCN(*)CC2(C)C)CC1]

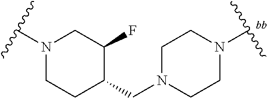 [SMILES: *N1CCN(C[C@@H]2CCN(*)C[C@H]2F)CC1]

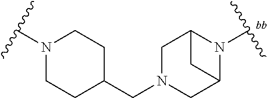 [SMILES: *N1CCC(CN2CC3CC(C2)N3*)CC1]

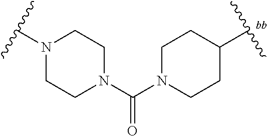 [SMILES: *C1CCN(C(=O)N2CCN(*)CC2)CC1]

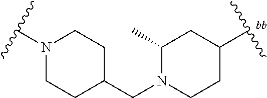 [SMILES: *C1CCN(CC2CCN(*)CC2)[C@H](C)C1]

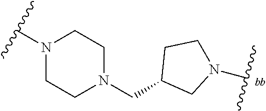 [SMILES: *N1CCN(C[C@@H]2CCN(*)C2)CC1]

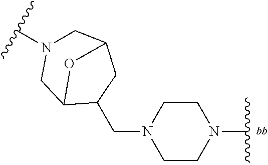 [SMILES: *N1CCN(CC2CC3CN(*)CC2O3)CC1]

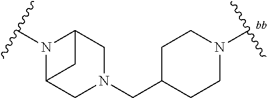 [SMILES: *N1CCC(CN2CC3CC(C2)N3*)CC1]

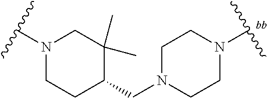 [SMILES: *N1CCN(C[C@@H]2CCN(*)CC2(C)C)CC1]

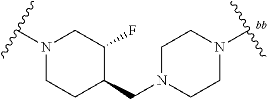 [SMILES: *N1CCN(C[C@H]2CCN(*)C[C@@H]2F)CC1]

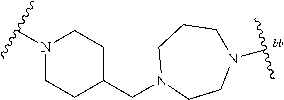 [SMILES: *N1CCC(CN2CCCN(*)CC2)CC1]

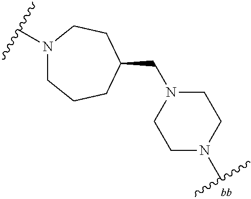 [SMILES: *N1CCC[C@H](CN2CCN(*)CC2)CC1]

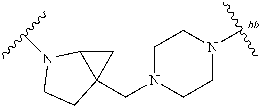 [SMILES: *N1CCN(CC23CCN(*)C2C3)CC1]

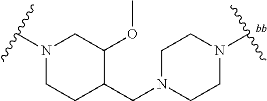 [SMILES: *N1CCN(CC2CCN(*)CC2OC)CC1]

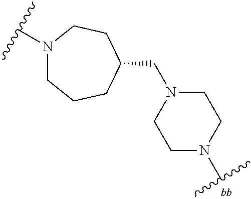 [SMILES: *N1CCC[C@@H](CN2CCN(*)CC2)CC1]

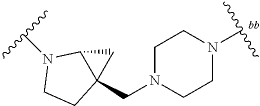 [SMILES: *N1CCN(C[C@@]23CCN(*)[C@@H]2C3)CC1]

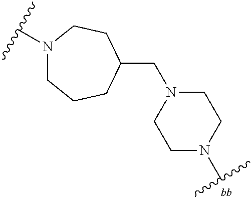 [SMILES: *N1CCCC(CN2CCN(*)CC2)CC1]

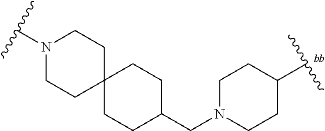 [SMILES: *C1CCN(CC2CCC3(CC2)CCN(*)CC3)CC1]

In some embodiments, the compounds of Formula (I-aa) are compounds of Formula (I-aa-1):

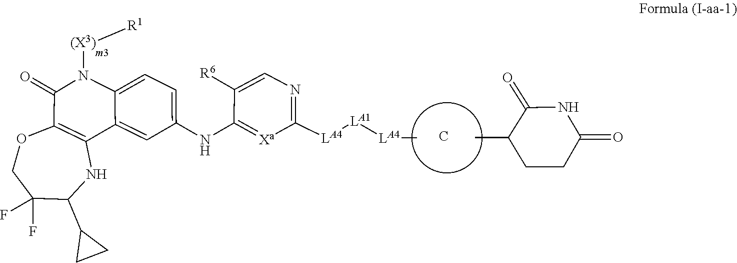

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

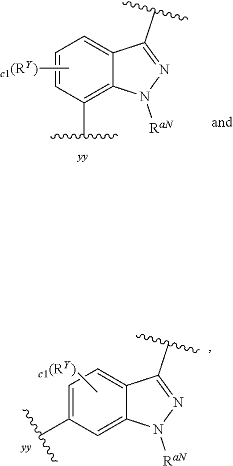

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa-1), the -L^{A4}-L^{A1}-L^{A4}- moiety is selected from the group consisting of the moieties depicted in Table L-I-a-1, wherein bb represents the point of attachment to Ring C.

TABLE L-I-a-1

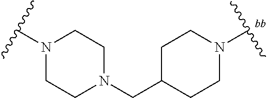 [SMILES: *N1CCC(CN2CCN(*)CC2)CC1]

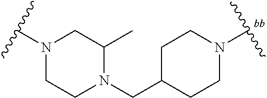 [SMILES: *N1CCC(CN2CCN(*)CC2C)CC1]

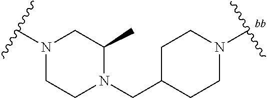 [SMILES: *N1CCC(CN2CCN(*)C[C@H]2C)CC1]

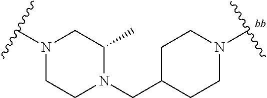 [SMILES: *N1CCC(CN2CCN(*)C[C@@H]2C)CC1]

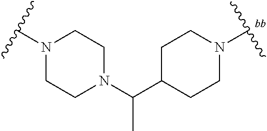 [SMILES: *N1CCC(C(C)N2CCN(*)CC2)CC1]

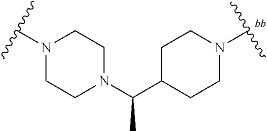 [SMILES: *N1CCC([C@@H](C)N2CCN(*)CC2)CC1]

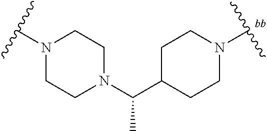 [SMILES: *N1CCC([C@H](C)N2CCN(*)CC2)CC1]

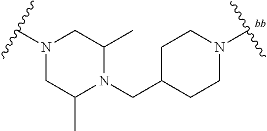 [SMILES: *N1CCC(CN2C(C)CN(*)CC2C)CC1]

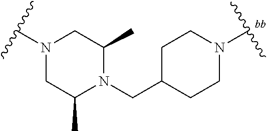 [SMILES: *N1CCC(CN2[C@H](C)CN(*)C[C@@H]2C)CC1]

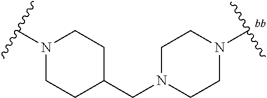 [SMILES: *N1CCC(CN2CCN(*)CC2)CC1]

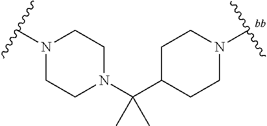 [SMILES: *N1CCC(C(C)(C)N2CCN(*)CC2)CC1]

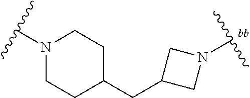 [SMILES: *N1CCC(CC2CN(*)C2)CC1]

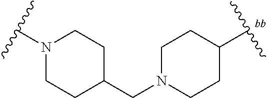 [SMILES: *C1CCN(CC2CCN(*)CC2)CC1]

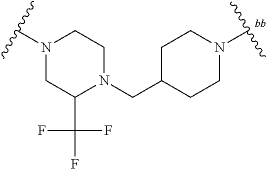 [SMILES: *N1CCC(CN2CCN(*)CC2C(F)(F)F)CC1]

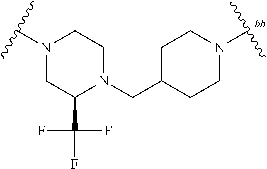 [SMILES: *N1CCC(CN2CCN(*)C[C@@H]2C(F)(F)F)CC1]

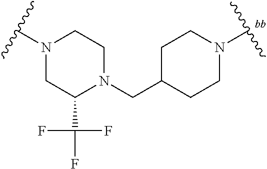 [SMILES: *N1CCC(CN2CCN(*)C[C@H]2C(F)(F)F)CC1]

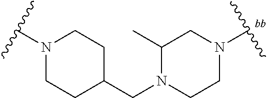 [SMILES: *N1CCC(CN2CCN(*)CC2C)CC1]

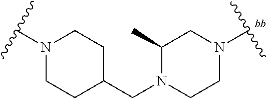 [SMILES: *N1CCC(CN2CCN(*)C[C@@H]2C)CC1]

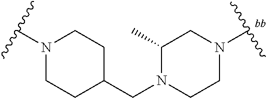 [SMILES: *N1CCC(CN2CCN(*)C[C@H]2C)CC1]

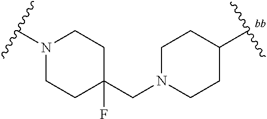 [SMILES: *C1CCN(CC2(F)CCN(*)CC2)CC1]

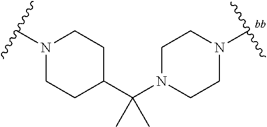 [SMILES: *N1CCC(C(C)(C)N2CCN(*)CC2)CC1]

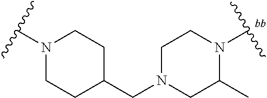 [SMILES: *N1CCC(CN2CCN(*)C(C)C2)CC1]

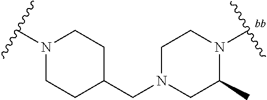 [SMILES: *N1CCC(CN2CCN(*)[C@@H](C)C2)CC1]

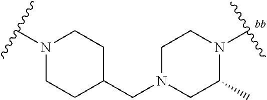 [SMILES: *N1CCC(CN2CCN(*)[C@H](C)C2)CC1]

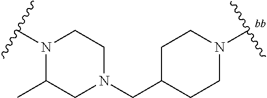 [SMILES: *N1CCC(CN2CCN(*)C(C)C2)CC1]

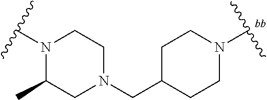 [SMILES: *N1CCC(CN2CCN(*)[C@H](C)C2)CC1]

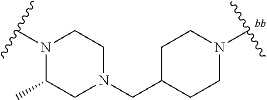 [SMILES: *N1CCC(CN2CCN(*)[C@@H](C)C2)CC1]

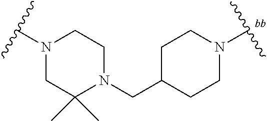 [SMILES: *N1CCC(CN2CCN(*)CC2(C)C)CC1]

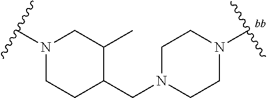 [SMILES: *N1CCN(CC2CCN(*)CC2C)CC1]

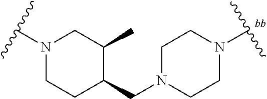 [SMILES: *N1CCN(C[C@H]2CCN(*)C[C@H]2C)CC1]

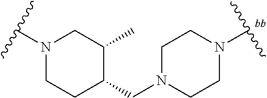 [SMILES: *N1CCN(C[C@@H]2CCN(*)C[C@@H]2C)CC1]

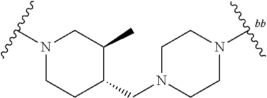 [SMILES: *N1CCN(C[C@@H]2CCN(*)C[C@H]2C)CC1]

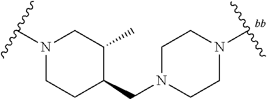 [SMILES: *N1CCN(C[C@H]2CCN(*)C[C@@H]2C)CC1]

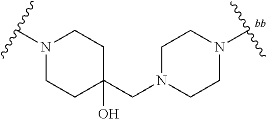 [SMILES: *N1CCN(CC2(O)CCN(*)CC2)CC1]

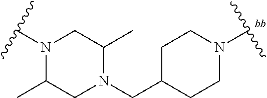 [SMILES: *N1CCC(CN2CC(C)N(*)CC2C)CC1]

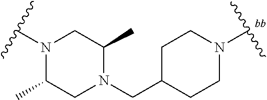 [SMILES: *N1CCC(CN2C[C@H](C)N(*)C[C@H]2C)CC1]

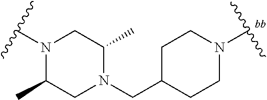 [SMILES: *N1CCC(CN2C[C@@H](C)N(*)C[C@@H]2C)CC1]

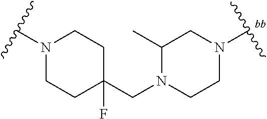 [SMILES: *N1CCC(F)(CN2CCN(*)CC2C)CC1]

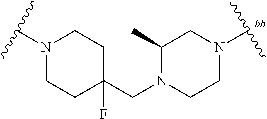 [SMILES: *N1CCC(F)(CN2CCN(*)C[C@@H]2C)CC1]

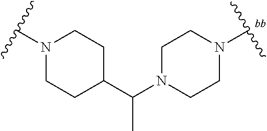 [SMILES: *N1CCC(C(C)N2CCN(*)CC2)CC1]

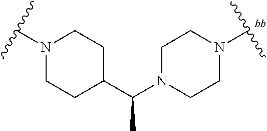 [SMILES: *N1CCC([C@H](C)N2CCN(*)CC2)CC1]

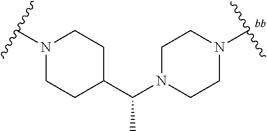 [SMILES: *N1CCC([C@@H](C)N2CCN(*)CC2)CC1]

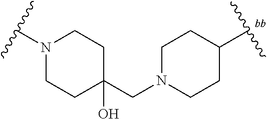 [SMILES: *C1CCN(CC2(O)CCN(*)CC2)CC1]

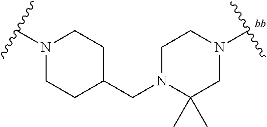 [SMILES: *N1CCC(CN2CCN(*)CC2(C)C)CC1]

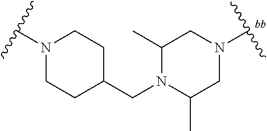 [SMILES: *N1CCC(CN2C(C)CN(*)CC2C)CC1]

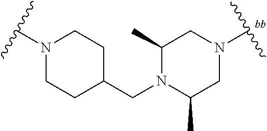 [SMILES: *N1CCC(CN2[C@H](C)CN(*)C[C@@H]2C)CC1]

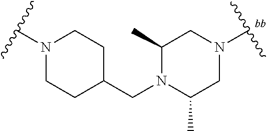 [SMILES: *N1CCC(CN2[C@@H](C)CN(*)C[C@@H]2C)CC1]

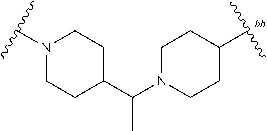 [SMILES: *C1CCN(C(C)C2CCN(*)CC2)CC1]

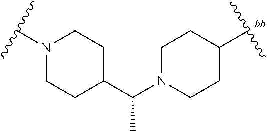 [SMILES: *C1CCN([C@H](C)C2CCN(*)CC2)CC1]

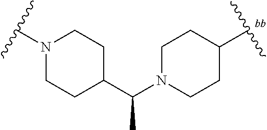 [SMILES: *C1CCN([C@@H](C)C2CCN(*)CC2)CC1]

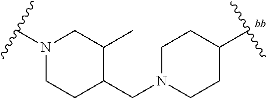 [SMILES: *C1CCN(CC2CCN(*)CC2C)CC1]

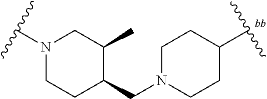 [SMILES: *C1CCN(C[C@H]2CCN(*)C[C@H]2C)CC1]

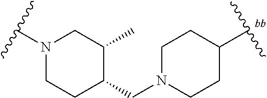 [SMILES: *C1CCN(C[C@@H]2CCN(*)C[C@@H]2C)CC1]

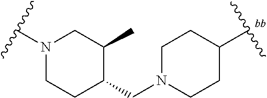 [SMILES: *C1CCN(C[C@@H]2CCN(*)C[C@H]2C)CC1]

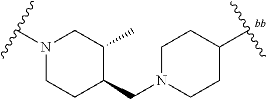 [SMILES: *C1CCN(C[C@H]2CCN(*)C[C@@H]2C)CC1]

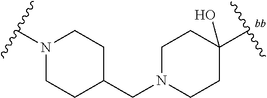 [SMILES: *N1CCC(CN2CCC(*)(O)CC2)CC1]

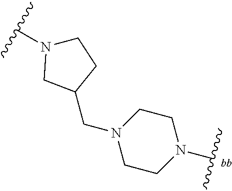 [SMILES: *N1CCN(CC2CCN(*)C2)CC1]

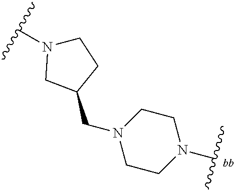 [SMILES: *N1CCN(C[C@@H]2CCN(*)C2)CC1]

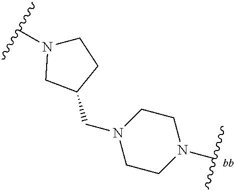 [SMILES: *N1CCN(C[C@H]2CCN(*)C2)CC1]

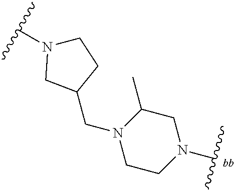 [SMILES: *N1CCC(CN2CCN(*)CC2C)C1]

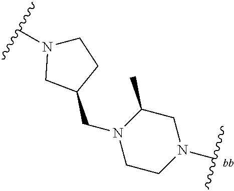 [SMILES: *N1CC[C@@H](CN2CCN(*)C[C@@H]2C)C1]

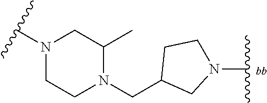 [SMILES: *N1CCC(CN2CCN(*)CC2C)C1]

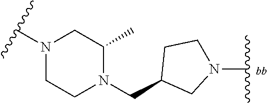 [SMILES: *N1CC[C@H](CN2CCN(*)C[C@@H]2C)C1]

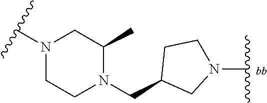 [SMILES: *N1CC[C@H](CN2CCN(*)C[C@H]2C)C1]

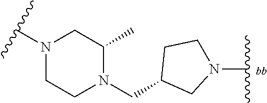 [SMILES: *N1CC[C@@H](CN2CCN(*)C[C@@H]2C)C1]

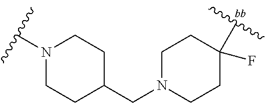 [SMILES: *N1CCC(CN2CCC(*)(F)CC2)CC1]

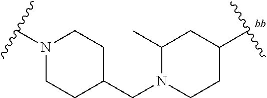 [SMILES: *C1CCN(CC2CCN(*)CC2)C(C)C1]

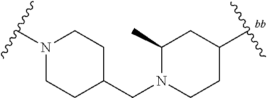 [SMILES: *C1CCN(CC2CCN(*)CC2)[C@@H](C)C1]

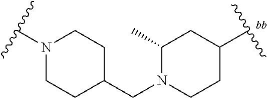 [SMILES: *C1CCN(CC2CCN(*)CC2)[C@H](C)C1]

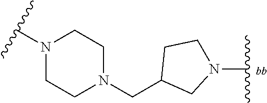 [SMILES: *N1CCN(CC2CCN(*)C2)CC1]

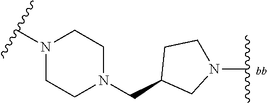 [SMILES: *N1CCN(C[C@H]2CCN(*)C2)CC1]

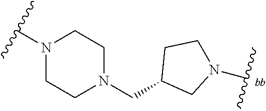 [SMILES: *N1CCN(C[C@@H]2CCN(*)C2)CC1]

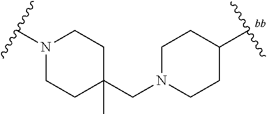 [SMILES: *C1CCN(CC2(C)CCN(*)CC2)CC1]

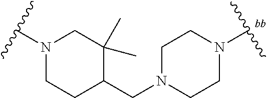 [SMILES: *N1CCN(CC2CCN(*)CC2(C)C)CC1]

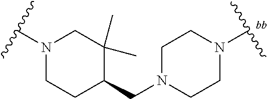 [SMILES: *N1CCN(C[C@H]2CCN(*)CC2(C)C)CC1]

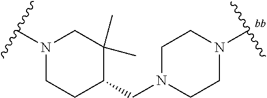 [SMILES: *N1CCN(C[C@@H]2CCN(*)CC2(C)C)CC1]

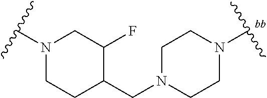 [SMILES: *N1CCN(CC2CCN(*)CC2F)CC1]

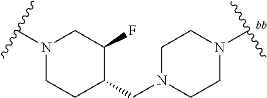 [SMILES: *N1CCN(C[C@@H]2CCN(*)C[C@H]2F)CC1]

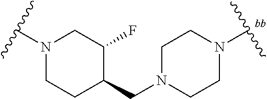 [SMILES: *N1CCN(C[C@H]2CCN(*)C[C@@H]2F)CC1]

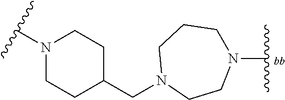 [SMILES: *N1CCC(CN2CCCN(*)CC2)CC1]

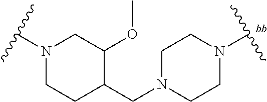 [SMILES: *N1CCN(CC2CCN(*)CC2OC)CC1]

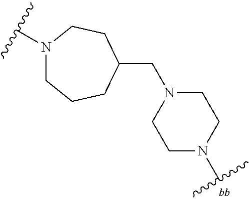 [SMILES: *N1CCCC(CN2CCN(*)CC2)CC1]

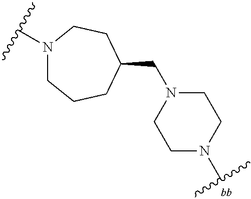 [SMILES: *N1CCC[C@H](CN2CCN(*)CC2)CC1]

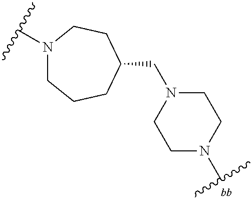 [SMILES: *N1CCC[C@@H](CN2CCN(*)CC2)CC1]

In some embodiments, the compounds of Formula (I-aa) are compounds of Formula (I-aa-2):

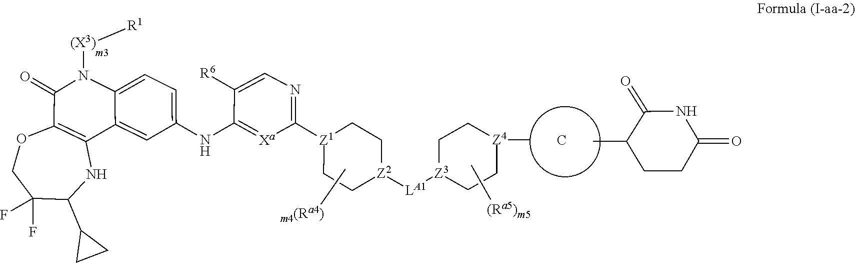

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

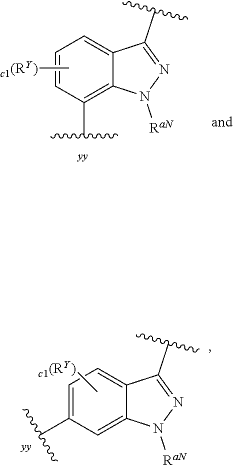

wherein: c1 is 0 or 1, R¹ is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N, provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F.

In some embodiments of Formula (I-aa-2), Z¹ is N. In some embodiments of Formula (I-aa-2), Z¹ is N; and Z² is CH or CR^{a4}. In some embodiments of Formula (I-aa-2), Z¹ is N; Z² is CH or CR^{a4}; and Z³ is N.

In some embodiments of Formula (I-aa-2), Z¹ is N; Z² is CH or CR^{a4}; Z³ is N; and Z⁴ is CH or CR^{a5}.

In some embodiments of Formula (I-aa-2), Z¹ is N; Z² is CH; Z³ is N; Z⁴ is CH; and m4 and m5 are both 0. In some embodiments of Formula (I-aa-2), Z¹ is N; Z² is CH; Z³ is N; Z⁴ is CH; one of m4 and m5 is 1; and the other of m4 and m5 is 0. In some embodiments of Formula (I-aa-2), Z¹ is N; Z² is CH; Z³ is N; Z⁴ is CH; one of m4 and m5 is 1; the other of m4 and m5 is 0; and the R^{a4} or R^{a5} when present is methyl. In some embodiments of Formula (I-aa-2), Z¹ is N; Z² is CH; Z³ is N; Z⁴ is CH; m4 is 1; and m5 is 0. In some embodiments of Formula (I-aa-2), Z¹ is N; Z² is CH; Z³ is N; Z⁴ is CH; m4 is 1; m5 is 0; and R^{a4} is methyl.

In some embodiments of Formula (I-aa-2), Z¹ is N; Z² is CH; Z³ is N; and Z⁴ is CR^{a5} (e.g., CF). In some embodiments of Formula (I-aa-2), Z¹ is N; Z² is CH; Z³ is N; Z⁴ is CF; and m4 and m5 are both 0.

In some embodiments of Formula (I-aa-2), Z¹ is N; Z² is CH or CR^{a4}; Z³ is N; and Z⁴ is N. In some embodiments of Formula (I-aa-2), Z¹ is N; Z² is CH; Z³ is N; Z⁴ is N; and m4 and m5 are both 0. In some embodiments of Formula (I-aa-2), Z¹ is N; Z² is CH; Z³ is N; Z⁴ is N; one of m4 and m5 is 1; and the other of m4 and m5 is 0. In some embodiments of Formula (I-aa-2), Z¹ is N; Z² is CH; Z³ is N; Z⁴ is N; one of m4 and m5 is 1; the other of m4 and m5 is 0; and the R^{a4} or R^{a5} when present is methyl. In some embodiments of Formula (I-aa-2), Z¹ is N; Z² is CH; Z³ is N; Z⁴ is N; m4 is 1; m5 is 0. In some embodiments, R^{a4} is methyl.

In some embodiments of Formula (I-aa-2), the

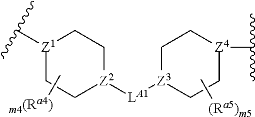

moiety is selected from the group consisting of the moieties depicted in Table L-I-a-2, wherein bb represents the point of attachment to Ring C.

TABLE L-I-a-2

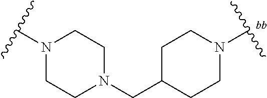 [SMILES: *N1CCC(CN2CCN(*)CC2)CC1]

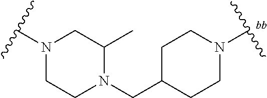 [SMILES: *N1CCC(CN2CCN(*)CC2C)CC1]

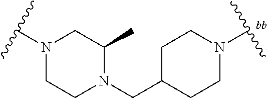 [SMILES: *N1CCC(CN2CCN(*)C[C@H]2C)CC1]

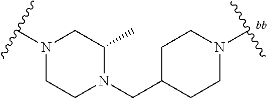 [SMILES: *N1CCC(CN2CCN(*)C[C@@H]2C)CC1]

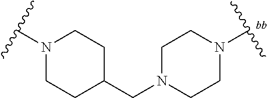 [SMILES: *N1CCC(CN2CCN(*)CC2)CC1]

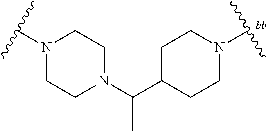 [SMILES: *N1CCC(C(C)N2CCN(*)CC2)CC1]

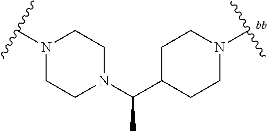 [SMILES: *N1CCC([C@@H](C)N2CCN(*)CC2)CC1]

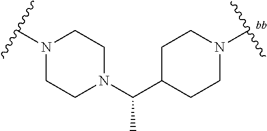 [SMILES: *N1CCC([C@H](C)N2CCN(*)CC2)CC1]

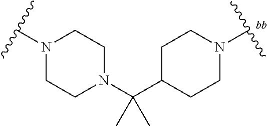 [SMILES: *N1CCC(C(C)(C)N2CCN(*)CC2)CC1]

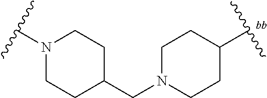 [SMILES: *C1CCN(CC2CCN(*)CC2)CC1]

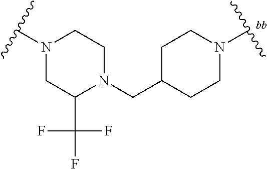 [SMILES: *N1CCC(CN2CCN(*)CC2C(F)(F)F)CC1]

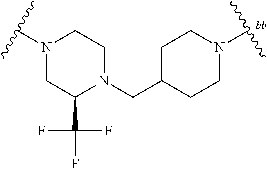 [SMILES: *N1CCC(CN2CCN(*)C[C@@H]2C(F)(F)F)CC1]

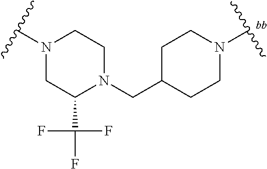 [SMILES: *N1CCC(CN2CCN(*)C[C@H]2C(F)(F)F)CC1]

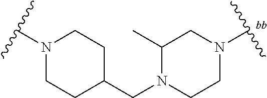 [SMILES: *N1CCC(CN2CCN(*)CC2C)CC1]

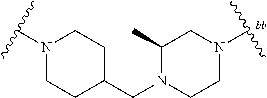 [SMILES: *N1CCC(CN2CCN(*)C[C@@H]2C)CC1]

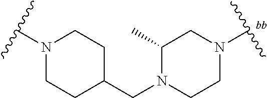 [SMILES: *N1CCC(CN2CCN(*)C[C@H]2C)CC1]

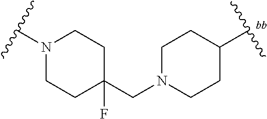 [SMILES: *C1CCN(CC2(F)CCN(*)CC2)CC1]

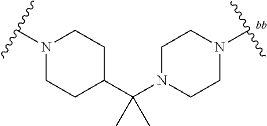 [SMILES: *N1CCC(C(C)(C)N2CCN(*)CC2)CC1]

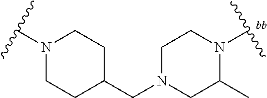 [SMILES: *N1CCC(CN2CCN(*)C(C)C2)CC1]

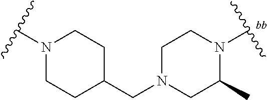 [SMILES: *N1CCC(CN2CCN(*)[C@@H](C)C2)CC1]

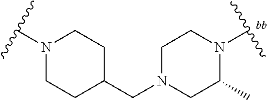 [SMILES: *N1CCC(CN2CCN(*)[C@H](C)C2)CC1]

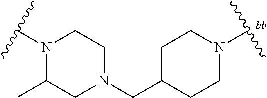 [SMILES: *N1CCC(CN2CCN(*)C(C)C2)CC1]

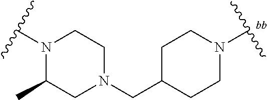 [SMILES: *N1CCC(CN2CCN(*)[C@H](C)C2)CC1]

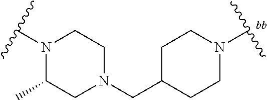 [SMILES: *N1CCC(CN2CCN(*)[C@@H](C)C2)CC1]

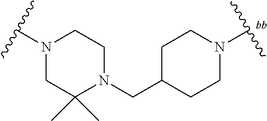 [SMILES: *N1CCC(CN2CCN(*)CC2(C)C)CC1]

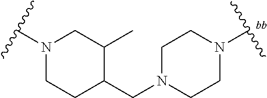 [SMILES: *N1CCN(CC2CCN(*)CC2C)CC1]

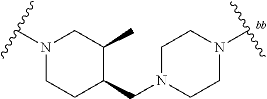 [SMILES: *N1CCN(C[C@H]2CCN(*)C[C@H]2C)CC1]

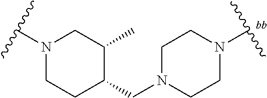 [SMILES: *N1CCN(C[C@@H]2CCN(*)C[C@@H]2C)CC1]

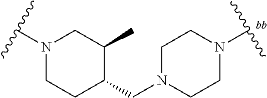 [SMILES: *N1CCN(C[C@@H]2CCN(*)C[C@H]2C)CC1]

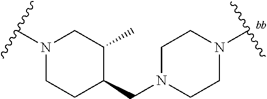 [SMILES: *N1CCN(C[C@H]2CCN(*)C[C@@H]2C)CC1]

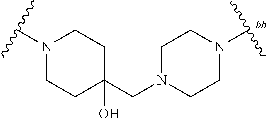 [SMILES: *N1CCN(CC2(O)CCN(*)CC2)CC1]

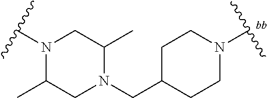 [SMILES: *N1CCC(CN2CC(C)N(*)CC2C)CC1]

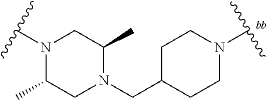 [SMILES: *N1CCC(CN2C[C@H](C)N(*)C[C@H]2C)CC1]

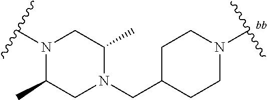 [SMILES: *N1CCC(CN2C[C@@H](C)N(*)C[C@@H]2C)CC1]

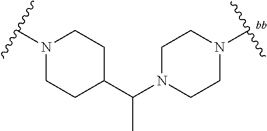 [SMILES: *N1CCC(C(C)N2CCN(*)CC2)CC1]

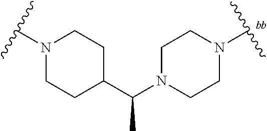 [SMILES: *N1CCC([C@H](C)N2CCN(*)CC2)CC1]

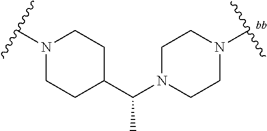 [SMILES: *N1CCC([C@@H](C)N2CCN(*)CC2)CC1]

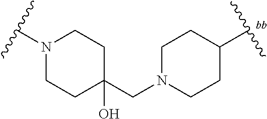 [SMILES: *C1CCN(CC2(O)CCN(*)CC2)CC1]

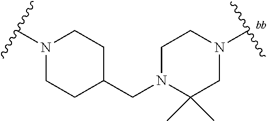 [SMILES: *N1CCC(CN2CCN(*)CC2(C)C)CC1]

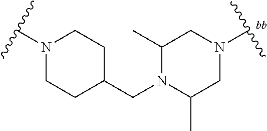 [SMILES: *N1CCC(CN2C(C)CN(*)CC2C)CC1]

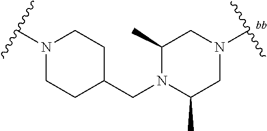 [SMILES: *N1CCC(CN2[C@H](C)CN(*)C[C@@H]2C)CC1]

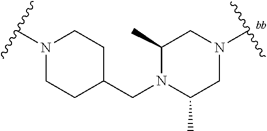 [SMILES: *N1CCC(CN2[C@@H](C)CN(*)C[C@@H]2C)CC1]

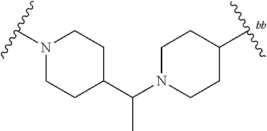 [SMILES: *C1CCN(C(C)C2CCN(*)CC2)CC1]

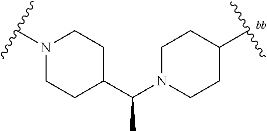 [SMILES: *C1CCN([C@@H](C)C2CCN(*)CC2)CC1]

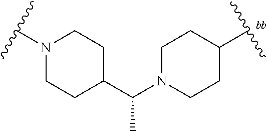 [SMILES: *C1CCN([C@H](C)C2CCN(*)CC2)CC1]

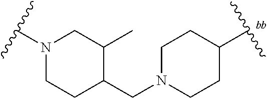 [SMILES: *C1CCN(CC2CCN(*)CC2C)CC1]

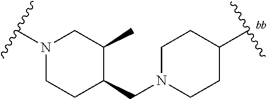 [SMILES: *C1CCN(C[C@H]2CCN(*)C[C@H]2C)CC1]

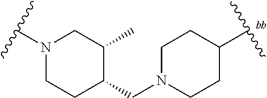 [SMILES: *C1CCN(C[C@@H]2CCN(*)C[C@@H]2C)CC1]

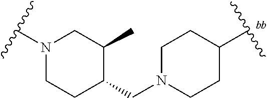 [SMILES: *C1CCN(C[C@@H]2CCN(*)C[C@H]2C)CC1]

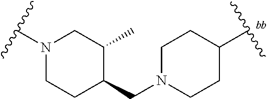 [SMILES: *C1CCN(C[C@H]2CCN(*)C[C@@H]2C)CC1]

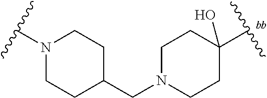 [SMILES: *N1CCC(CN2CCC(*)(O)CC2)CC1]

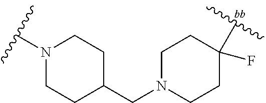 [SMILES: *N1CCC(CN2CCC(*)(F)CC2)CC1]

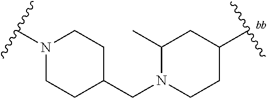 [SMILES: *C1CCN(CC2CCN(*)CC2)C(C)C1]

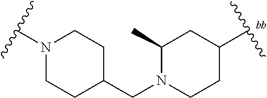 [SMILES: *C1CCN(CC2CCN(*)CC2)[C@@H](C)C1]

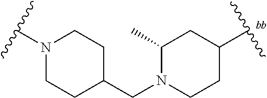 [SMILES: *C1CCN(CC2CCN(*)CC2)[C@H](C)C1]

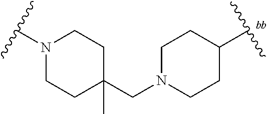 [SMILES: *C1CCN(CC2(C)CCN(*)CC2)CC1]

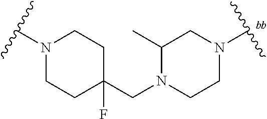 [SMILES: *N1CCC(F)(CN2CCN(*)CC2C)CC1]

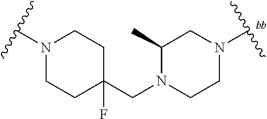 [SMILES: *N1CCC(F)(CN2CCN(*)C[C@@H]2C)CC1]

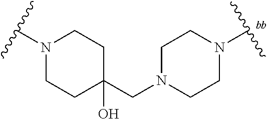 [SMILES: *N1CCN(CC2(O)CCN(*)CC2)CC1]

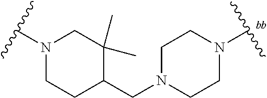 [SMILES: *N1CCN(CC2CCN(*)CC2(C)C)CC1]

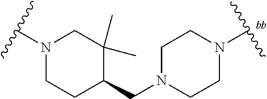 [SMILES: *N1CCN(C[C@H]2CCN(*)CC2(C)C)CC1]

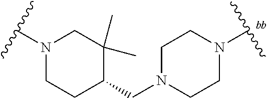 [SMILES: *N1CCN(C[C@@H]2CCN(*)CC2(C)C)CC1]

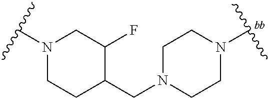 [SMILES: *N1CCN(CC2CCN(*)CC2F)CC1]

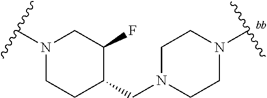 [SMILES: *N1CCN(C[C@@H]2CCN(*)C[C@H]2F)CC1]

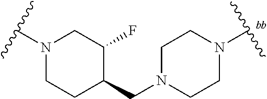 [SMILES: *N1CCN(C[C@H]2CCN(*)C[C@@H]2F)CC1]

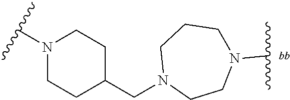 [SMILES: *N1CCC(CN2CCCN(*)CC2)CC1]

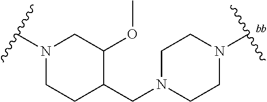 [SMILES: *N1CCN(CC2CCN(*)CC2OC)CC1]

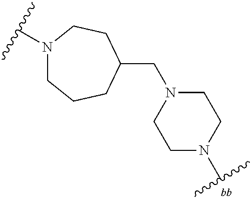 [SMILES: *N1CCCC(CN2CCN(*)CC2)CC1]

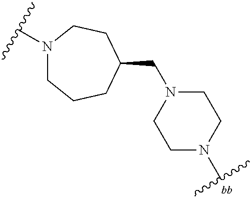 [SMILES: *N1CCC[C@H](CN2CCN(*)CC2)CC1]

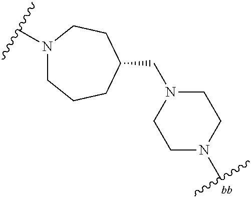 [SMILES: *N1CCC[C@@H](CN2CCN(*)CC2)CC1]

In some embodiments, the compounds of Formula (I-aa) are compounds of Formula (I-aa-3):

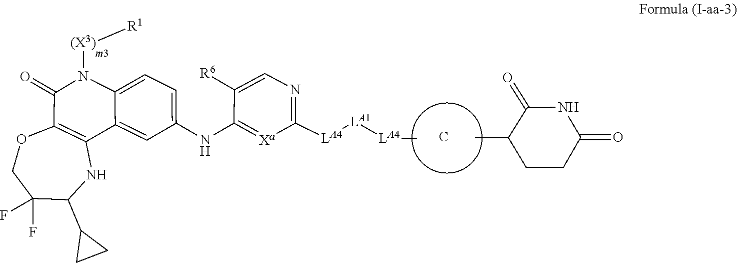

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

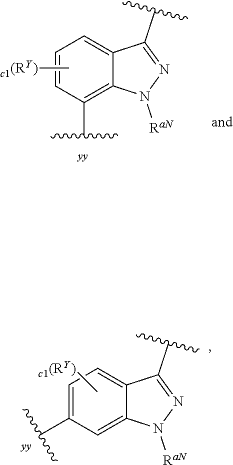

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa-3), each L^{A4} contains 1-2 ring nitrogen atoms and no additional ring heteroatom.

In some embodiments of Formula (I-aa-3), one L^{A4} is a monocyclic 4-6 membered nitrogen-containing heterocyclylene optionally substituted with 1-3 R^{a}; and the other L^{A4} is a bicyclic spirocyclic 6-12 (e.g., 7, 9, or 11) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-aa-3), one L^{A4} is a monocyclic 4-6 membered nitrogen-containing heterocyclylene optionally substituted with 1-3 R^{a}; and the other L^{A4} is a bicyclic bridged 6-12 (e.g., 7, 8, or 9) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-aa-3), one L^{A4} is a monocyclic 4-6 membered nitrogen-containing heterocyclylene optionally substituted with 1-3 R^{a}; and the other L^{A4} is a bicyclic fused 6-12 (e.g., 6) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-aa-3), the -L^{A4}-L^{A1}-L^{A4}- moiety is selected from the group consisting of the moieties depicted in Table L-I-a-3, wherein bb represents the point of attachment to Ring C.

TABLE L-I-a-3

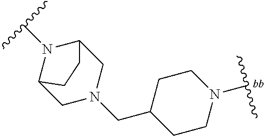 [SMILES: *N1CCC(CN2CC3CCC(C2)N3*)CC1]

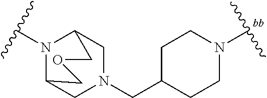 [SMILES: *N1CCC(CN2CC3COCC(C2)N3*)CC1]

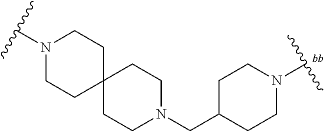 [SMILES: *N1CCC(CN2CCC3(CCN(*)CC3)CC2)CC1]

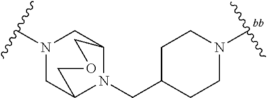 [SMILES: *N1CCC(CN2C3COCC2CN(*)C3)CC1]

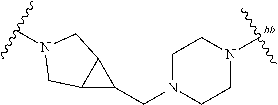 [SMILES: *N1CCN(CC2C3CN(*)CC32)CC1]

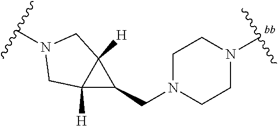 [SMILES: *N1CCN(C[C@H]2[C@@H]3CN(*)C[C@@H]32)CC1]

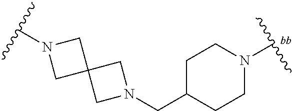 [SMILES: *N1CCC(CN2CC3(CN(*)C3)C2)CC1]

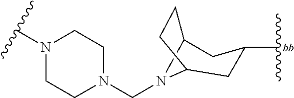 [SMILES: *C1CC2CCC(C1)N2CN1CCN(*)CC1]

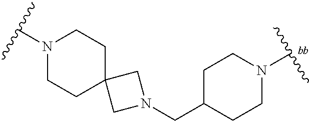 [SMILES: *N1CCC(CN2CC3(CCN(*)CC3)C2)CC1]

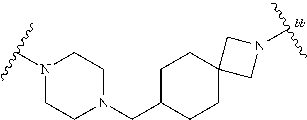 [SMILES: *N1CCN(CC2CCC3(CC2)CN(*)C3)CC1]

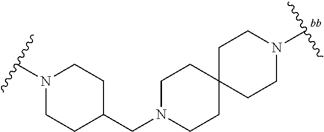 [SMILES: *N1CCC(CN2CCC3(CCN(*)CC3)CC2)CC1]

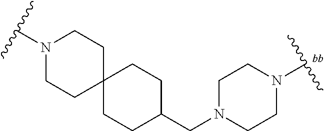 [SMILES: *N1CCN(CC2CCC3(CC2)CCN(*)CC3)CC1]

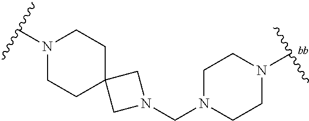 [SMILES: *N1CCN(CN2CC3(CCN(*)CC3)C2)CC1]

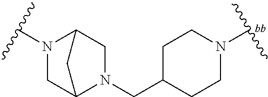 [SMILES: *N1CCC(CN2CC3CC2CN3*)CC1]

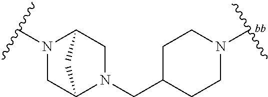 [SMILES: *N1CCC(CN2C[C@H]3C[C@@H]2CN3*)CC1]

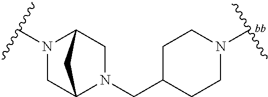 [SMILES: *N1CCC(CN2C[C@@H]3C[C@H]2CN3*)CC1]

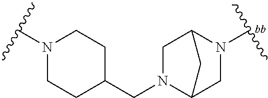 [SMILES: *N1CCC(CN2CC3CC2CN3*)CC1]

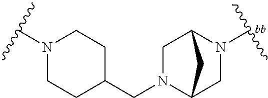 [SMILES: *N1CCC(CN2C[C@H]3C[C@@H]2CN3*)CC1]

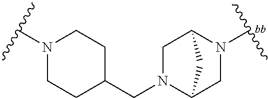 [SMILES: *N1CCC(CN2C[C@@H]3C[C@H]2CN3*)CC1]

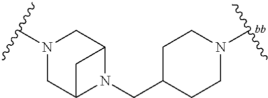 [SMILES: *N1CCC(CN2C3CC2CN(*)C3)CC1]

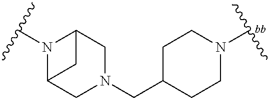 [SMILES: *N1CCC(CN2CC3CC(C2)N3*)CC1]

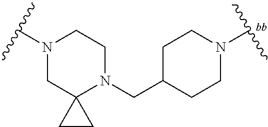 [SMILES: *N1CCC(CN2CCN(*)CC23CC3)CC1]

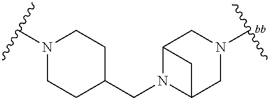 [SMILES: *N1CCC(CN2C3CC2CN(*)C3)CC1]

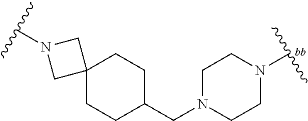 [SMILES: *N1CCN(CC2CCC3(CC2)CN(*)C3)CC1]

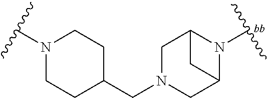 [SMILES: *N1CCC(CN2CC3CC(C2)N3*)CC1]

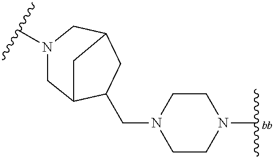 [SMILES: *N1CCN(CC2CC3CC2CN(*)C3)CC1]

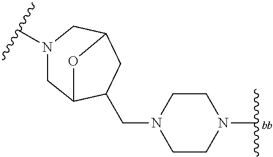 [SMILES: *N1CCN(CC2CC3CN(*)CC2O3)CC1]

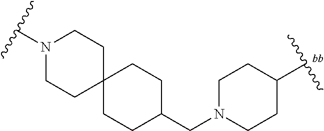 [SMILES: *C1CCN(CC2CCC3(CC2)CCN(*)CC3)CC1]

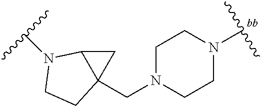 [SMILES: *N1CCN(CC23CCN(*)C2C3)CC1]

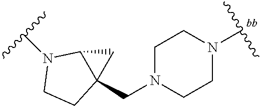 [SMILES: *N1CCN(C[C@@]23CCN(*)[C@@H]2C3)CC1]

In some embodiments, the compounds of Formula (I-aa) are compounds of Formula (I-aa-4):

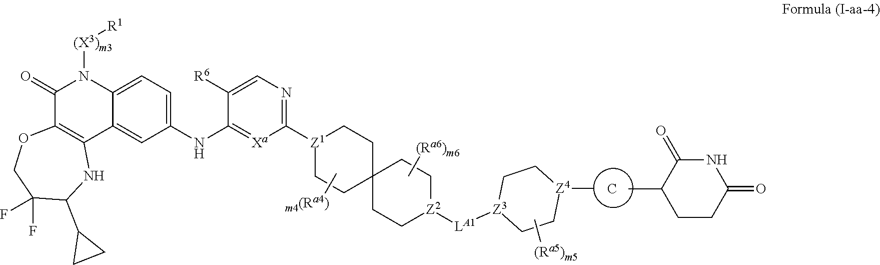

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

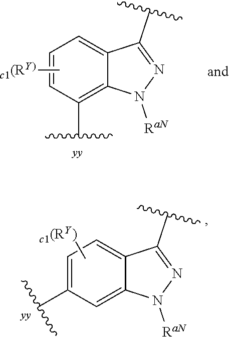

wherein: c1 is 0 or 1, R¹ is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

In some embodiments of Formula (I-aa-4), Z¹ is N. In some embodiments of Formula (I-aa-4), Z¹ is N; and Z² is N. In some embodiments of Formula (I-aa-4), Z¹ is N; Z² is N; Z³ is CH; and Z⁴ is N. In some embodiments of Formula (I-aa-4), Z¹ is N; Z² is N; Z³ is CH; Z⁴ is N; and 2-3 (e.g., 3) of m4, m6, and m6 are 0.

In some embodiments of Formula (I-aa-4), the

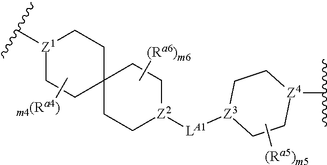

moiety is selected from the group consisting of the moieties depicted in Table L-I-a-4, wherein bb represents the point of attachment to Ring C.

TABLE L-I-a-4

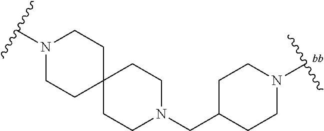 [SMILES: *N1CCC(CN2CCC3(CCN(*)CC3)CC2)CC1]

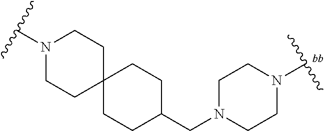 [SMILES: *N1CCN(CC2CCC3(CC2)CCN(*)CC3)CC1]

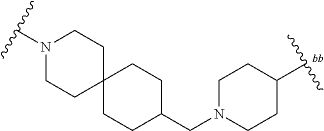 [SMILES: *C1CCN(CC2CCC3(CC2)CCN(*)CC3)CC1]

In some embodiments, the compounds of Formula (I-aa) are compounds of Formula (I-aa-5):

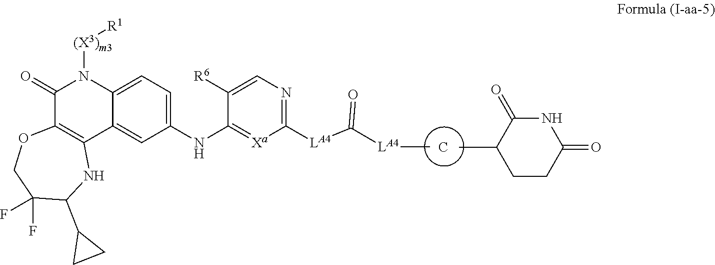

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

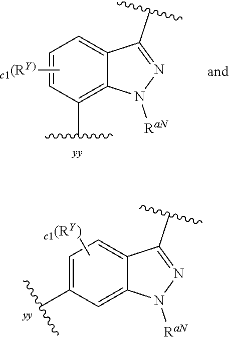

wherein: c1 is 0 or 1, R^{Y} is selected from the group consisting of halo (e.g., —F) and C₁₋₃ alkyl optionally substituted with 1-3 F, and R^{aN} is C₁₋₃ alkyl; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa-5), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene and piperidinylene.

In some embodiments of Formula (I-aa-5), the -L^{A4}-C(═O)-L^{A4}- moiety is selected from the group consisting of the moieties depicted in Table L-I-a-5, wherein bb represents the point of attachment to Ring C.

TABLE L-I-a-5

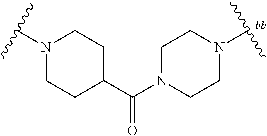 [SMILES: *N1CCC(C(=O)N2CCN(*)CC2)CC1]

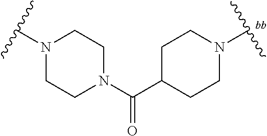 [SMILES: *N1CCC(C(=O)N2CCN(*)CC2)CC1]

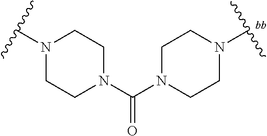 [SMILES: *N1CCN(C(=O)N2CCN(*)CC2)CC1]

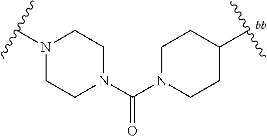 [SMILES: *C1CCN(C(=O)N2CCN(*)CC2)CC1]

In some embodiments, the compounds of Formula (I-aa) are compounds of Formula (I-aa-6):

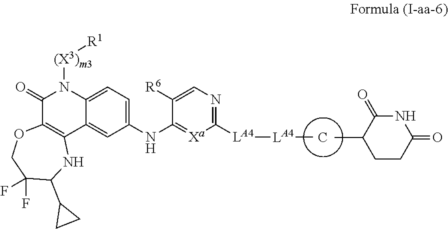

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

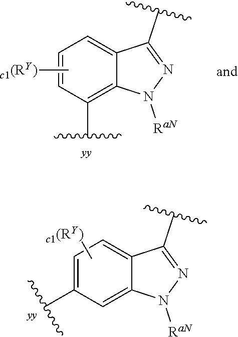

wherein: c1 is 0 or 1, R^{Y} is selected from the group consisting of halo (e.g., —F) and C₁₋₃ alkyl optionally substituted with 1-3 F, and R^{aN} is C₁₋₃ alkyl; and
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa-6), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene, piperidinylene, and pyrrolidinylene.

In some embodiments of Formula (I-aa-6), the -L^{A4}-L^{A4}- moiety is selected from the group consisting of the moieties depicted in Table L-I-a-6, wherein bb represents the point of attachment to Ring C.

TABLE L-I-a-6

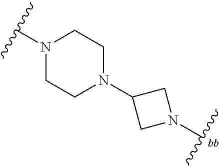

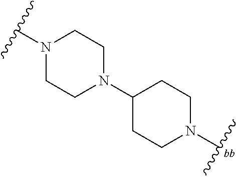

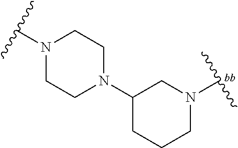

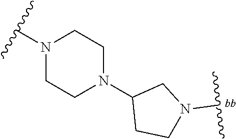

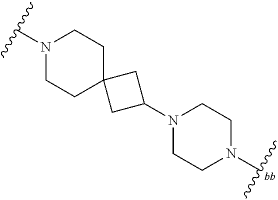

In some embodiments, the compounds of Formula (I) are compounds of Formula (I-a):

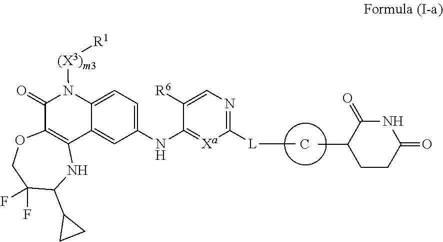

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

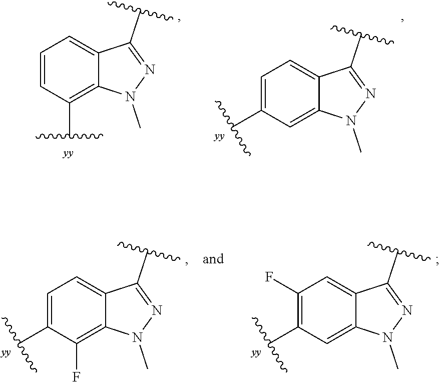 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L is selected from the group consisting of:
   L^{A4}-L^{A1}-L^{A4}-_{bb};
   L^{A4}-L^{A1}-L^{A1}-L^{A4}-_{bb};
   L^{A4}-L^{A4}-_{bb};
   L^{A4}-C(═O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms.

In some embodiments of Formula (I-a), one L^{A4} is a monocyclic 4-6 membered nitrogen-containing heterocyclylene optionally substituted with 1-3 R^{a}; and the other L^{A4} is a bicyclic spirocyclic 6-12 (e.g., 6-10) membered nitrogen-containing heterocyclylene optionally substituted with 1-3 R^{a}, wherein each L^{A4} contains 1-2 ring nitrogen atoms and no additional ring heteroatoms.

In some embodiments of Formula (I-a), L is -L^{A4}-L^{A1}-L^{A4}-_{bb}; and L^{A1} is CH₂ or CHMe.

In some embodiments of Formula (I-a), L is selected from the group consisting of the moieties delineated in Table Li-a.

In some embodiments of Formula (I-a), Ring C is selected from the group consisting of:

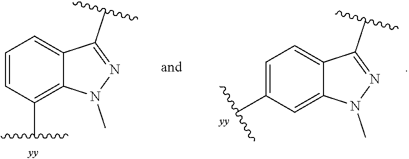 [SMILES: *c1nn(C)c2c(*)cccc12 ; *c1ccc2c(*)nn(C)c2c1]

In some embodiments of Formula (I-a), L is selected from the group consisting of the moieties depicted in Table L-I-a (supra).

In some embodiments, the compounds of Formula (I-a) are compounds of Formula (I-a-1):

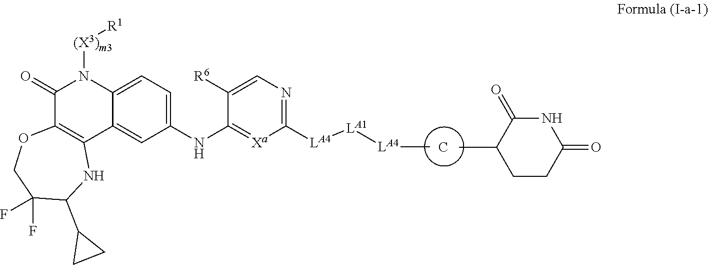

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

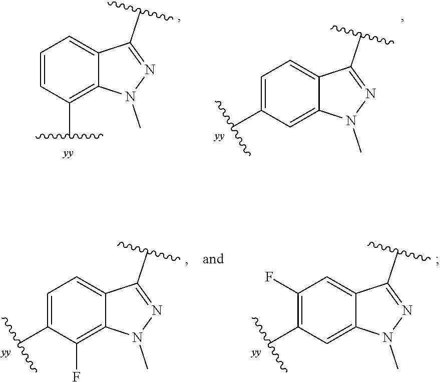 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-1), the -L^{A4}-L^{A1}-L^{A4}- moiety is selected from the group consisting of the moieties depicted in Table L-I-a-1 (supra), wherein bb represents the point of attachment to Ring C.

In some embodiments, the compounds of Formula (I-a) are compounds of Formula (I-a-2):

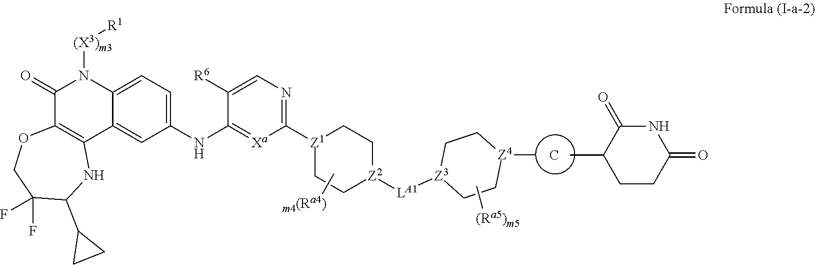

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

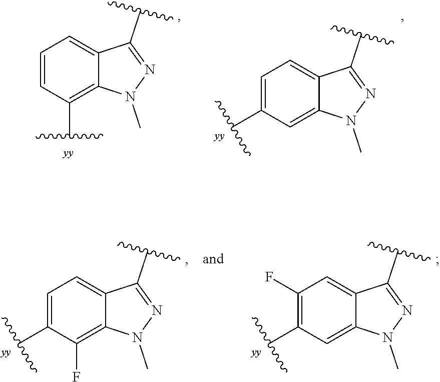 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and Rs is independently selected from the group consisting of: —F, CN, C1-3 alkoxy, OH, and C₁₋₃ alkyl optionally substituted with 1-3 F.

In some embodiments of Formula (I-aa-2), Z¹ is N. In some embodiments of Formula (I-aa-2), Z¹ is N; and Z² is CH or CR^{a4}. In some embodiments of Formula (I-aa-2), Z¹ is N; Z² is CH or CR^{a4}; and Z³ is N.

In some embodiments of Formula (I-aa-2), Z¹ is N; Z² is CH or CR^{a4}; Z³ is N; and Z⁴ is CH or CR^{a5}.

In some embodiments of Formula (I-aa-2), Z¹ is N; Z² is CH; Z³ is N; Z⁴ is CH; and m4 and m5 are both 0. In some embodiments of Formula (I-aa-2), Z¹ is N; Z² is CH; Z³ is N; Z⁴ is CH; one of m4 and m5 is 1; and the other of m4 and m5 is 0. In some embodiments of Formula (I-aa-2), Z¹ is N; Z² is CH; Z³ is N; Z⁴ is CH; one of m4 and m5 is 1; the other of m4 and m5 is 0; and the R^{a4} or R^{a5} when present is methyl. In some embodiments of Formula (I-aa-2), Z¹ is N; Z² is CH; Z³ is N; Z⁴ is CH; m4 is 1; and m5 is 0. In some embodiments of Formula (I-aa-2), Z¹ is N; Z² is CH; Z³ is N; Z⁴ is CH; m4 is 1; m5 is 0; and R^{a4} is methyl.

In some embodiments of Formula (I-aa-2), Z¹ is N; Z² is CH; Z³ is N; and Z⁴ is CR^{a5} (e.g., CF). In some embodiments of Formula (I-aa-2), Z¹ is N; Z² is CH; Z³ is N; Z⁴ is CF; and m4 and m5 are both 0.

In some embodiments of Formula (I-aa-2), Z¹ is N; Z² is CH or CR^{a4}; Z³ is N; and Z⁴ is N. In some embodiments of Formula (I-aa-2), Z¹ is N; Z² is CH; Z³ is N; Z⁴ is N; and m4 and m5 are both 0. In some embodiments of Formula (I-aa-2), Z¹ is N; Z² is CH; Z³ is N; Z⁴ is N; one of m4 and m5 is 1; and the other of m4 and m5 is 0. In some embodiments of Formula (I-aa-2), Z¹ is N; Z² is CH; Z³ is N; Z⁴ is N; one of m4 and m5 is 1; the other of m4 and m5 is 0; and the R^{a4} or R^{a5} when present is methyl. In some embodiments of Formula (I-aa-2), Z¹ is N; Z² is CH; Z³ is N; Z⁴ is N; m4 is 1; m5 is 0. In some embodiments, R^{a4} is methyl.

In some embodiments of Formula (I-a-2), the

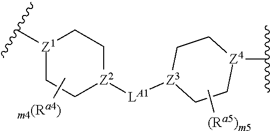

moiety is selected from the group consisting of the moieties depicted in Table L-I-a-2 (supra), wherein bb represents the point of attachment to Ring C.

In some embodiments, the compounds of Formula (I-a) are compounds of Formula (I-a-3):

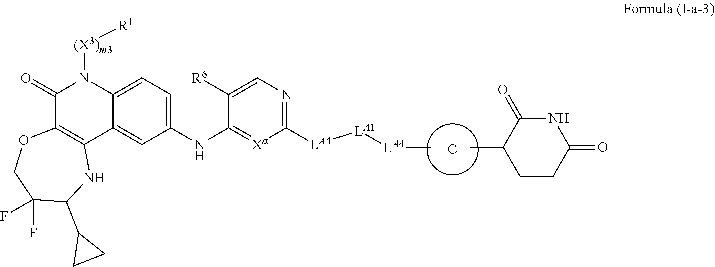

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

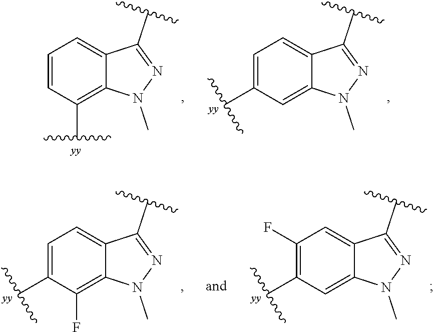 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂;
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-3), one L^{A4} is a monocyclic 4-6 membered nitrogen-containing heterocyclylene optionally substituted with 1-3 R^{a}; and the other L^{A4} is a bicyclic spirocyclic 6-12 (e.g., 7, 9, or 11) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-a-3), one L^{A4} is a monocyclic 4-6 membered nitrogen-containing heterocyclylene optionally substituted with 1-3 R^{a}; and the other L^{A4} is a bicyclic bridged 6-12 (e.g., 7, 8, or 9) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-a-3), one L^{A4} is a monocyclic 4-6 membered nitrogen-containing heterocyclylene optionally substituted with 1-3 R^{a}; and the other L^{A4} is a bicyclic fused 6-12 (e.g., 6) membered nitrogen-containing heterocyclylene optionally substituted with 1-3 R^{a}.

In some embodiments, each L^{A4} contains 1-2 ring nitrogen atoms and no additional ring heteroatom.

In some embodiments of Formula (I-a-3), the -L^{A4}-L^{A1}-L^{A4}- moiety is selected from the group consisting of the moieties depicted in Table L-I-a-3 (supra), wherein bb represents the point of attachment to Ring C.

In some embodiments, the compounds of Formula (I-a) are compounds of Formula (I-a-4):

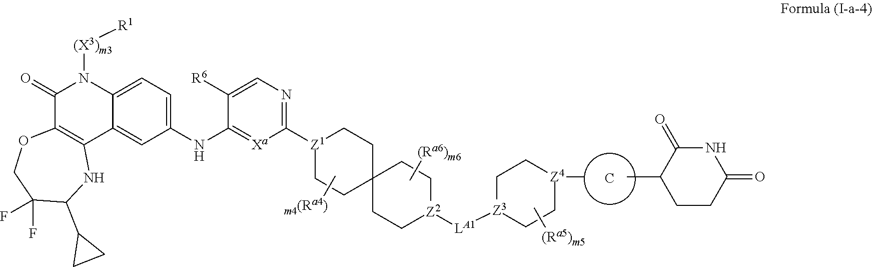

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

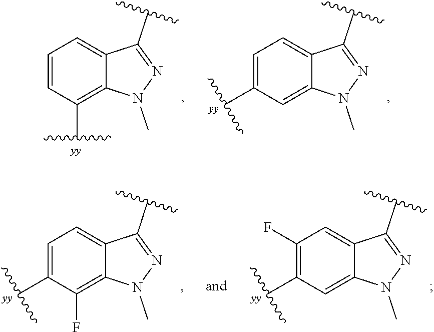 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

In some embodiments of Formula (I-a4), Z¹ is N; and Z² is N. In some embodiments of Formula (I-a4), Z¹ is N; Z² is N; Z³ is CH; and Z⁴ is N. In some embodiments of Formula (I-a4), Z¹ is N; Z² is N; Z³ is CH; Z⁴ is N; and 2-3 of m4, m6, and m6 are 0.

In some embodiments of Formula (I-a-4), the

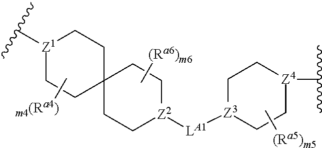

moiety is selected from the group consisting of the moieties depicted in Table L-I-a-4 (supra), wherein bb represents the point of attachment to Ring C.

In some embodiments, the compounds of Formula (I-a) are compounds of Formula (I-a-5):

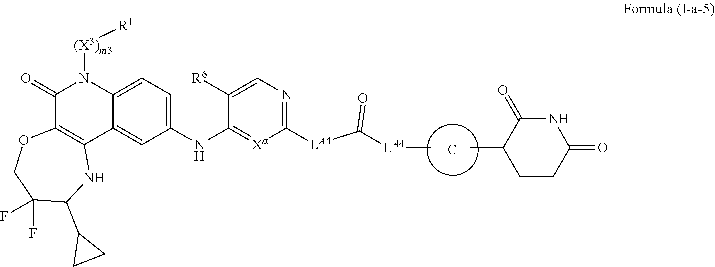

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

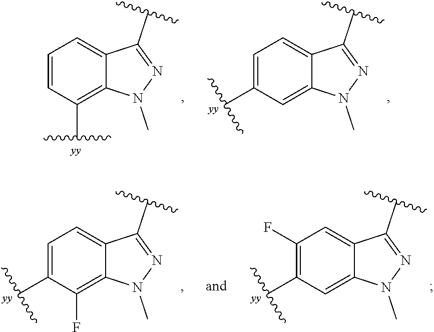 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

and
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-5), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene and piperidinylene.

In some embodiments of Formula (I-a-5), the -L^{A4}-C(═O)-L^{A4}- moiety is selected from the group consisting of the moieties depicted in Table L-I-a-5 (supra), wherein bb represents the point of attachment to Ring C.

In some embodiments, the compounds of Formula (I-a) are compounds of Formula (I-a-6):

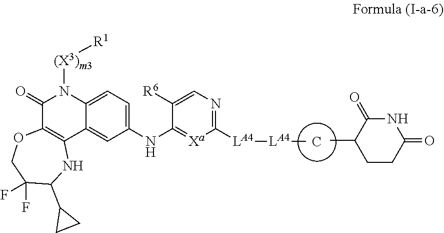

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

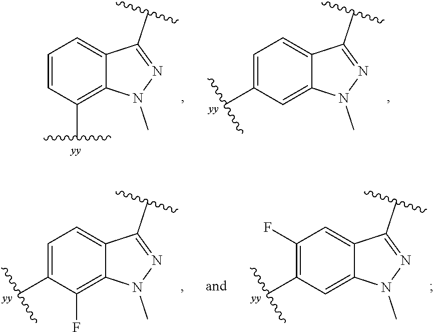 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-6), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene, piperidinylene, and pyrrolidinylene.

In some embodiments of Formula (I-a-6), the -L^{A4}-L^{A4}- moiety is selected from the group consisting of the moieties depicted in Table L-I-a-6 (supra), wherein bb represents the point of attachment to Ring C.

In some embodiments, the compounds of Formula (I) are compounds of Formula (I-bb):

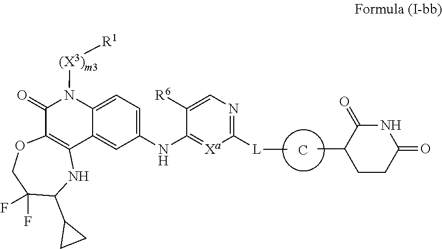

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

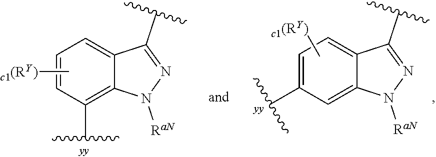

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is -L^{A4}-L^{A3}-_{bb} or -L^{A4}-L^{A1}-L^{A3}-_{bb}, wherein bb represents the point of attachment to Ring C; and
L^{A4} is a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-bb), L is -L^{A4}-L^{A3}-_{bb}, and L^{A3} is —NH—.

In some embodiments of Formula (I-bb), L^{A4} is a monocyclic 4-6 membered nitrogen-containing heterocyclylene optionally substituted with 1-3 R^{a}, wherein L^{A4} contains 1-2 ring nitrogen atoms and no additional ring heteroatoms.

In some embodiments of Formula (I-bb), L^{A4} is a bicyclic spirocyclic 6-12 (e.g., 6-10) membered nitrogen-containing heterocyclylene optionally substituted with 1-3 R^{a}, wherein L^{A4} contains 1-2 ring nitrogen atoms and no additional ring heteroatoms.

In some embodiments of Formula (I-bb), X^{a} is N.

In some embodiments of Formula (I-bb), X^{a} is CH.

In some embodiments of Formula (I-bb), L is selected from the group consisting of the moieties depicted in Table L-I-b.

TABLE L-I-b

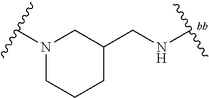 [SMILES: *NCC1CCCN(*)C1]

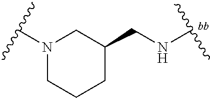 [SMILES: *NC[C@H]1CCCN(*)C1]

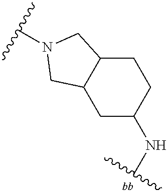 [SMILES: *NC1CCC2CN(*)CC2C1]

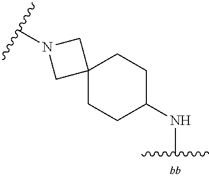 [SMILES: *NC1CCC2(CC1)CN(*)C2]

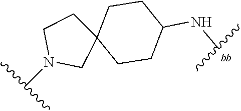 [SMILES: *NC1CCC2(CC1)CCN(*)C2]

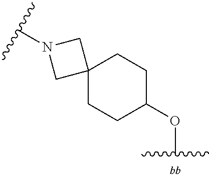 [SMILES: *OC1CCC2(CC1)CN(*)C2]

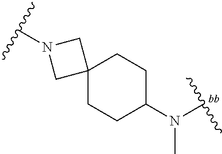 [SMILES: *N1CC2(CCC(N(*)C)CC2)C1]

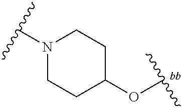 [SMILES: *OC1CCN(*)CC1]

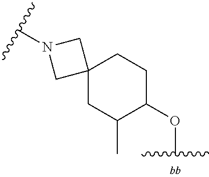 [SMILES: *OC1CCC2(CC1C)CN(*)C2]

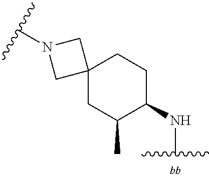 [SMILES: *N[C@@H]1CCC2(C[C@@H]1C)CN(*)C2]

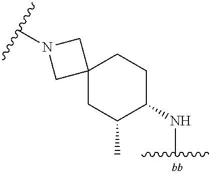 [SMILES: *N[C@H]1CCC2(C[C@H]1C)CN(*)C2]

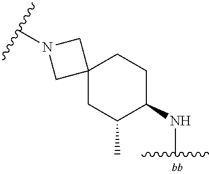 [SMILES: *N[C@@H]1CCC2(C[C@H]1C)CN(*)C2]

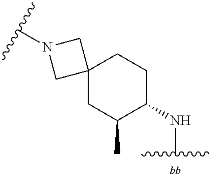 [SMILES: *N[C@H]1CCC2(C[C@@H]1C)CN(*)C2]

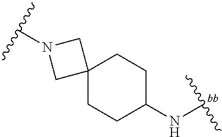 [SMILES: *NC1CCC2(CC1)CN(*)C2]

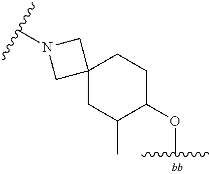 [SMILES: *OC1CCC2(CC1C)CN(*)C2]

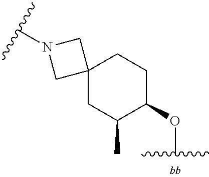 [SMILES: *O[C@@H]1CCC2(C[C@@H]1C)CN(*)C2]

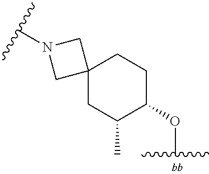 [SMILES: *O[C@H]1CCC2(C[C@H]1C)CN(*)C2]

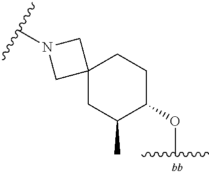 [SMILES: *O[C@H]1CCC2(C[C@@H]1C)CN(*)C2]

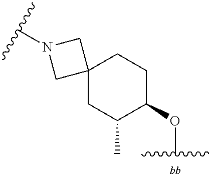 [SMILES: *O[C@@H]1CCC2(C[C@H]1C)CN(*)C2]

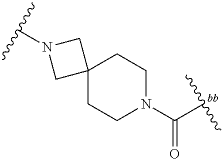 [SMILES: *C(=O)N1CCC2(CC1)CN(*)C2]

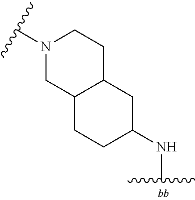 [SMILES: *NC1CCC2CN(*)CCC2C1]

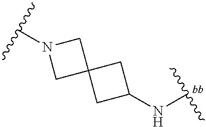 [SMILES: *NC1CC2(C1)CN(*)C2]

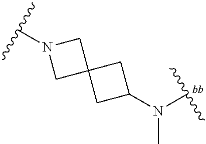 [SMILES: *N1CC2(CC(N(*)C)C2)C1]

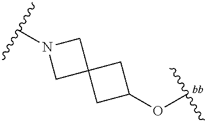 [SMILES: *OC1CC2(C1)CN(*)C2]

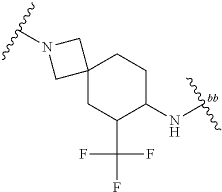 [SMILES: *NC1CCC2(CC1C(F)(F)F)CN(*)C2]

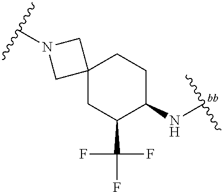 [SMILES: *N[C@@H]1CCC2(C[C@@H]1C(F)(F)F)CN(*)C2]

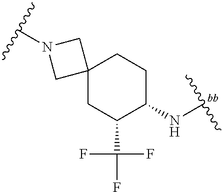 [SMILES: *N[C@H]1CCC2(C[C@H]1C(F)(F)F)CN(*)C2]

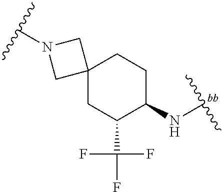 [SMILES: *N[C@@H]1CCC2(C[C@H]1C(F)(F)F)CN(*)C2]

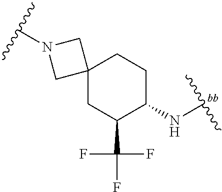 [SMILES: *N[C@H]1CCC2(C[C@@H]1C(F)(F)F)CN(*)C2]

In some embodiments, the compounds of Formula (I-bb) are compounds of Formula (I-bb-1):

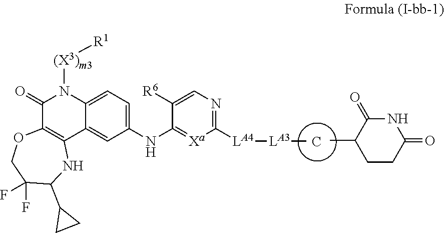

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

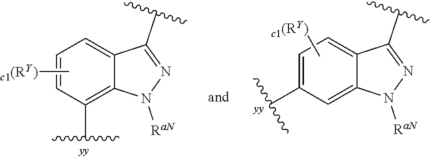

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A4} is 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; and
L^{A3} is —NH—, —N(C₁₋₃ alkyl)-, or —O—.

In some embodiments of Formula (I-bb-1), L^{A4} is a bicyclic spirocyclic 6-12 (e.g., 8-10 (e.g., 9)) membered nitrogen-containing heterocyclylene optionally substituted with 1-3 R^{a}, wherein L^{A4} contains 1-2 (e.g., 1) ring nitrogen atoms and no additional ring heteroatoms.

In some embodiments of Formula (I-bb-1), the L^{A4}-L^{A3} moiety is selected from the group consisting of the moieties depicted in Table L-I-b-1, wherein bb represents the point of attachment to Ring C.

TABLE L-I-b-1

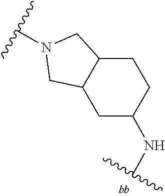 [SMILES: *NC1CCC2CN(*)CC2C1]

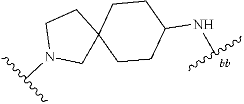 [SMILES: *NC1CCC2(CC1)CCN(*)C2]

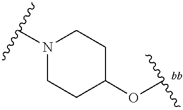 [SMILES: *OC1CCN(*)CC1]

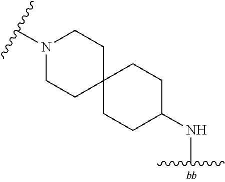 [SMILES: *NC1CCC2(CC1)CCN(*)CC2]

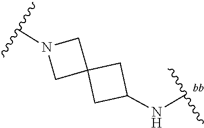 [SMILES: *NC1CC2(C1)CN(*)C2]

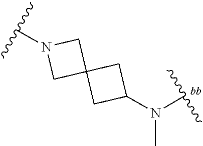 [SMILES: *N1CC2(CC(N(*)C)C2)C1]

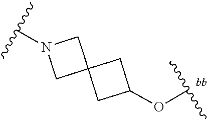 [SMILES: *OC1CC2(C1)CN(*)C2]

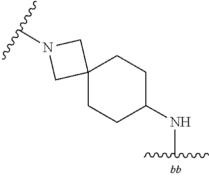 [SMILES: *NC1CCC2(CC1)CN(*)C2]

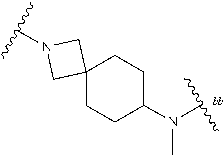 [SMILES: *N1CC2(CCC(N(*)C)CC2)C1]

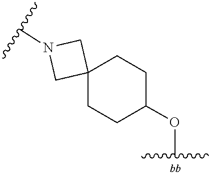 [SMILES: *OC1CCC2(CC1)CN(*)C2]

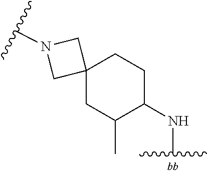 [SMILES: *NC1CCC2(CC1C)CN(*)C2]

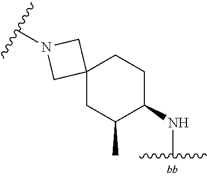 [SMILES: *N[C@@H]1CCC2(C[C@@H]1C)CN(*)C2]

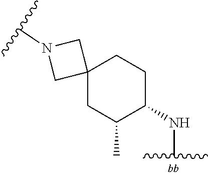 [SMILES: *N[C@H]1CCC2(C[C@H]1C)CN(*)C2]

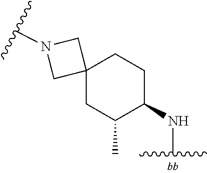 [SMILES: *N[C@@H]1CCC2(C[C@H]1C)CN(*)C2]

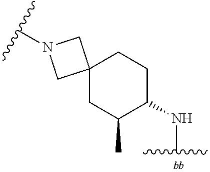

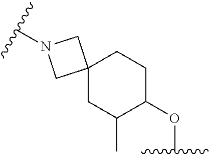 [SMILES: *OC1CCC2(CC1C)CN(*)C2]

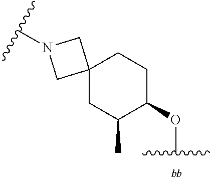 [SMILES: *O[C@@H]1CCC2(C[C@@H]1C)CN(*)C2]

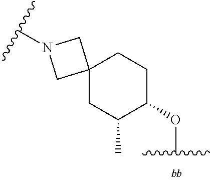 [SMILES: *O[C@H]1CCC2(C[C@H]1C)CN(*)C2]

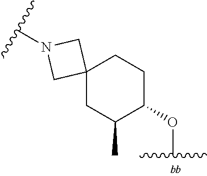 [SMILES: *O[C@H]1CCC2(C[C@@H]1C)CN(*)C2]

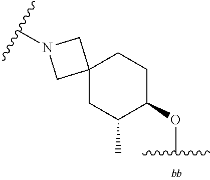 [SMILES: *O[C@@H]1CCC2(C[C@H]1C)CN(*)C2]

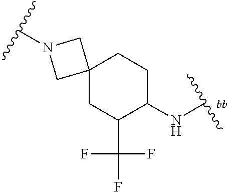 [SMILES: *NC1CCC2(CC1C(F)(F)F)CN(*)C2]

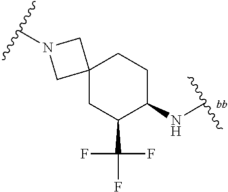 [SMILES: *N[C@@H]1CCC2(C[C@@H]1C(F)(F)F)CN(*)C2]

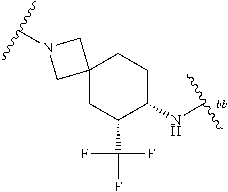 [SMILES: *N[C@H]1CCC2(C[C@H]1C(F)(F)F)CN(*)C2]

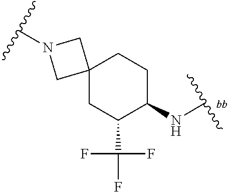 [SMILES: *N[C@@H]1CCC2(C[C@H]1C(F)(F)F)CN(*)C2]

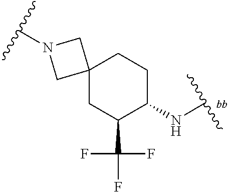 [SMILES: *N[C@H]1CCC2(C[C@@H]1C(F)(F)F)CN(*)C2]

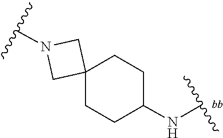 [SMILES: *NC1CCC2(CC1)CN(*)C2]

In some embodiments, the compounds of Formula (I-bb) are compounds of Formula (I-bb-2):

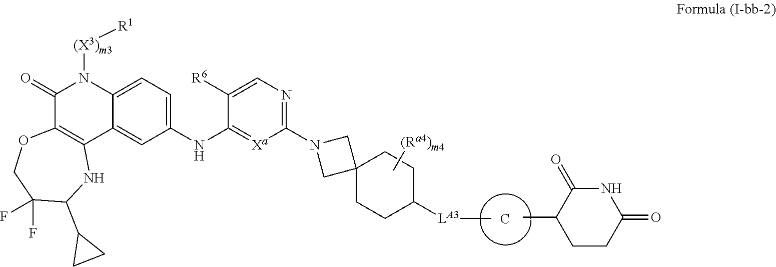

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

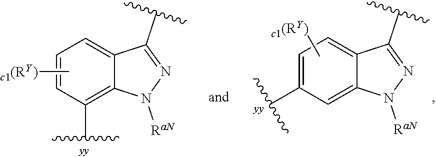

wherein: c1 is 0 or 1, R is selected from the group consisting of halo (e.g., —F) and C₁₋₃ alkyl optionally substituted with 1-3 F, and R^{aN} is C₁₋₃ alkyl;
L^{A3} is —NH—, —N(C₁₋₃ alkyl)-, or —O—;
m4 is selected from the group consisting of: 0, 1, and 2; and
each R^{a4} is independently selected from the group consisting of: —F, CN, C₁₋₃ alkoxy, OH, and C₁₋₃ alkyl optionally substituted with 1-3 F.

In some embodiments of Formula (I-bb-2), m4 is 0 or 1; and R^{a4} when present is methyl.

In some embodiments of Formula (I-bb-2), the

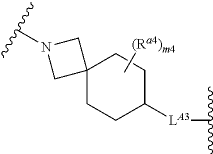

moiety is selected from the group consisting of the moieties depicted in Table L-I-b-2, wherein bb represents the point of attachment to Ring C.

TABLE L-I-b-2

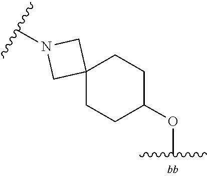 [SMILES: *OC1CCC2(CC1)CN(*)C2]

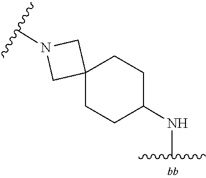 [SMILES: *NC1CCC2(CC1)CN(*)C2]

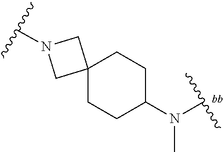 [SMILES: *N1CC2(CCC(N(*)C)CC2)C1]

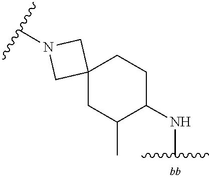 [SMILES: *NC1CCC2(CC1C)CN(*)C2]

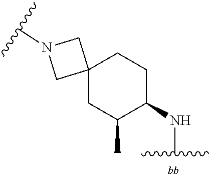 [SMILES: *N[C@@H]1CCC2(C[C@@H]1C)CN(*)C2]

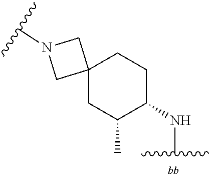 [SMILES: *N[C@H]1CCC2(C[C@H]1C)CN(*)C2]

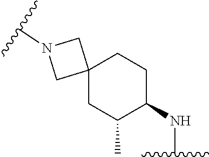 [SMILES: *N[C@@H]1CCC2(C[C@H]1C)CN(*)C2]

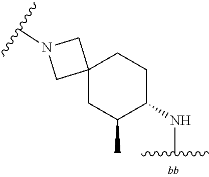 [SMILES: *N[C@H]1CCC2(C[C@@H]1C)CN(*)C2]

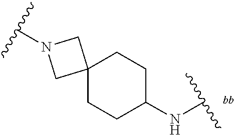 [SMILES: *NC1CCC2(CC1)CN(*)C2]

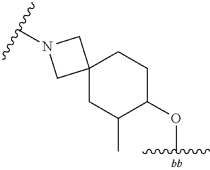 [SMILES: *OC1CCC2(CC1C)CN(*)C2]

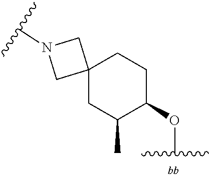 [SMILES: *O[C@@H]1CCC2(C[C@@H]1C)CN(*)C2]

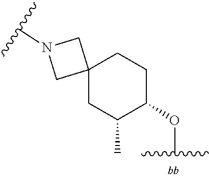 [SMILES: *O[C@H]1CCC2(C[C@H]1C)CN(*)C2]

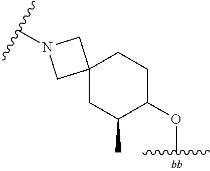 [SMILES: *OC1CCC2(C[C@@H]1C)CN(*)C2]

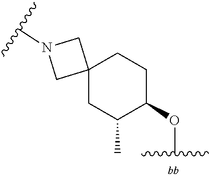 [SMILES: *O[C@@H]1CCC2(C[C@H]1C)CN(*)C2]

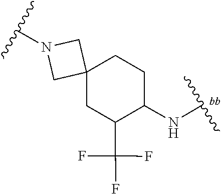 [SMILES: *NC1CCC2(CC1C(F)(F)F)CN(*)C2]

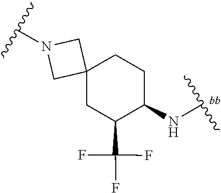 [SMILES: *N[C@@H]1CCC2(C[C@@H]1C(F)(F)F)CN(*)C2]

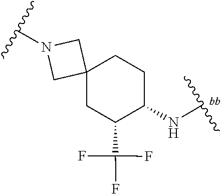 [SMILES: *N[C@H]1CCC2(C[C@H]1C(F)(F)F)CN(*)C2]

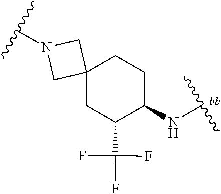 [SMILES: *N[C@@H]1CCC2(C[C@H]1C(F)(F)F)CN(*)C2]

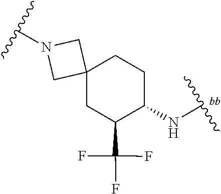 [SMILES: *N[C@H]1CCC2(C[C@@H]1C(F)(F)F)CN(*)C2]

In some embodiments, the compounds of Formula (I) are compounds of Formula (I-b):

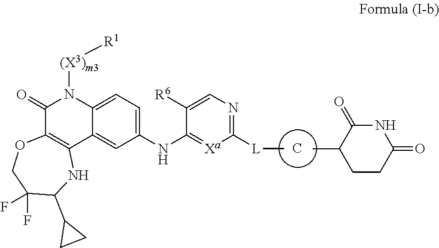

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

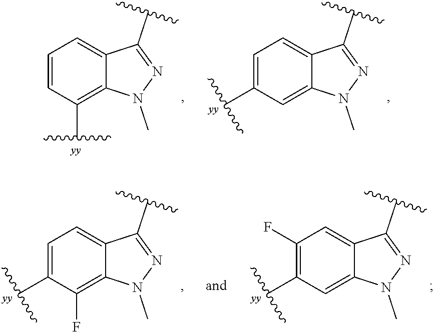 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L is -L^{A4}-L^{A3}-_{bb} or -L^{A4}-L^{A1}-L^{A3}-_{bb}, wherein bb represents the point of attachment to Ring C; and
L^{A4} is 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-b), L is -L^{A4}-L^{A3}-_{bb}, and L^{A3} is —NH—.

In some embodiments of Formula (I-b), L^{A4} is monocyclic 4-6 membered nitrogen-containing heterocyclylene optionally substituted with 1-3 R^{a}, wherein L^{A4} contains 1-2 ring nitrogen atoms and no additional ring heteroatoms.

In some embodiments of Formula (I-b), L^{A4} is bicyclic spirocyclic 6-12 (e.g., 6-10) membered nitrogen-containing heterocyclylene optionally substituted with 1-3 R^{a}, wherein L^{A4} contains 1-2 ring nitrogen atoms and no additional ring heteroatoms.

In some embodiments of Formula (I-b), X^{a} is N.

In some embodiments of Formula (I-b), X^{a} is CH.

In some embodiments of Formula (I-b), Ring C is selected from the group consisting of:

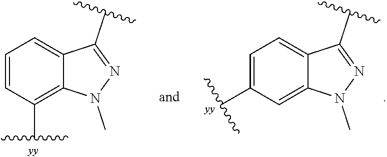 [SMILES: *c1nn(C)c2c(*)cccc12 ; *c1ccc2c(*)nn(C)c2c1]

In some embodiments of Formula (I-b), L is selected from the group consisting of the moieties depicted in Table L-I-b (supra).

In some embodiments, the compounds of Formula (I-b) are compounds of Formula (I-b-1):

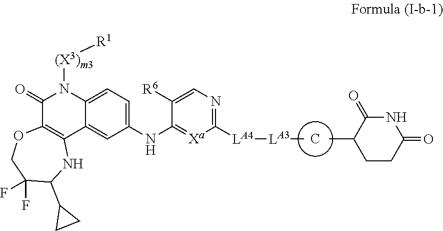

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

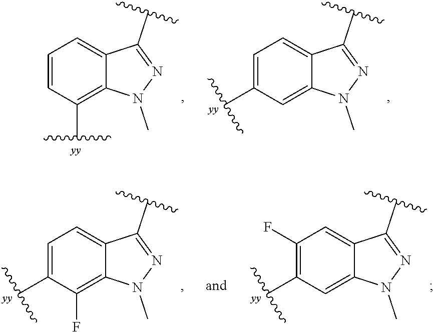 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A4} is 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; and
L^{A3} is —NH—, —N(C₁₋₃ alkyl)-, or —O—.

In some embodiments of Formula (I-b-1), L^{A4} is a bicyclic spirocyclic 6-12 (e.g., 8-10 (e.g., 9)) membered nitrogen-containing heterocyclylene optionally substituted with 1-3 R^{a}, wherein L^{A4} contains 1-2 (e.g., 1) ring nitrogen atoms and no additional ring heteroatoms.

In some embodiments of Formula (I-b-1), the L^{A4}-L^{A3} moiety is selected from the group consisting of the moieties depicted in Table L-I-b-1 (supra), wherein bb represents the point of attachment to Ring C.

In some embodiments, the compounds of Formula (I-bb) are compounds of Formula (I-b-2):

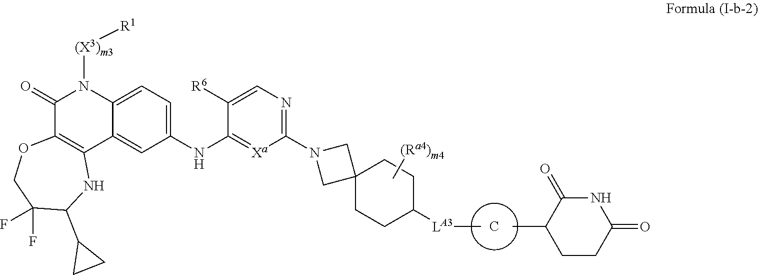

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

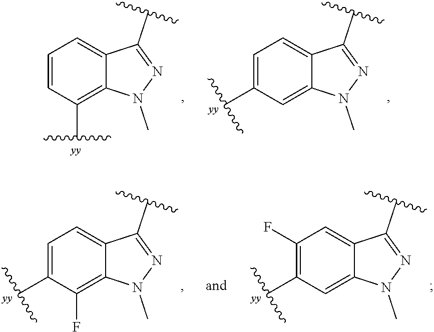 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A3} is —NH—, —N(C1-3 alkyl)-, or —O—;
m4 is selected from the group consisting of: 0, 1, and 2; and
each R^{a4} is independently selected from the group consisting of: —F, CN, C₁₋₃ alkoxy, OH, and C₁₋₃ alkyl optionally substituted with 1-3 F.

In some embodiments of Formula (I-b-2), m4 is 0 or 1; and R^{a4} when present is methyl.

In some embodiments of Formula (I-b-2), the

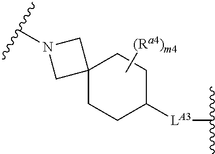

moiety is selected from the group consisting of the moieties depicted in Table L-I-b-2 (supra), wherein bb represents the point of attachment to Ring C.

In some embodiments, the compounds of Formula (I) are selected from the group consisting of the compounds in Table C1, or pharmaceutically acceptable salts thereof.

TABLE C1

| No. | Compound Structure |
|---|---|
| 101 | 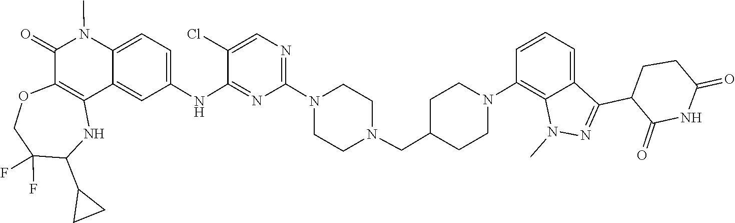 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)CC4)CC3)c21] |
| 101a | 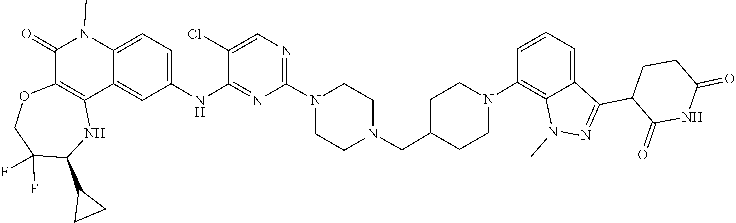 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)CC4)CC3)c21] |
| 102 | 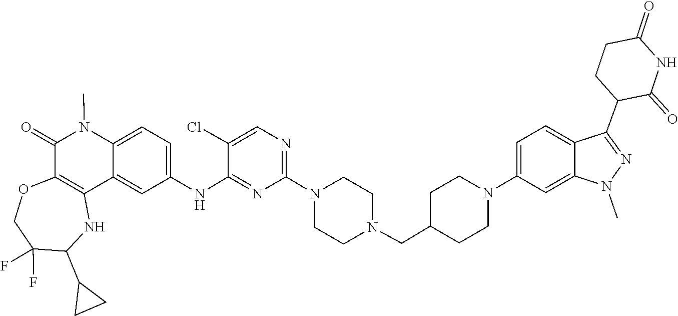 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)C(C7CC7)N6)n5)CC4)CC3)cc21] |
| 102a | 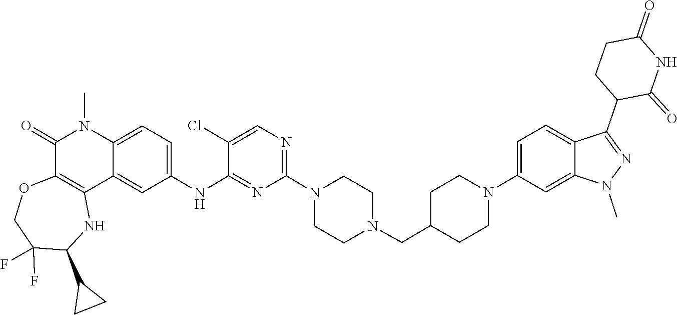 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)CC4)CC3)cc21] |
| 130 | 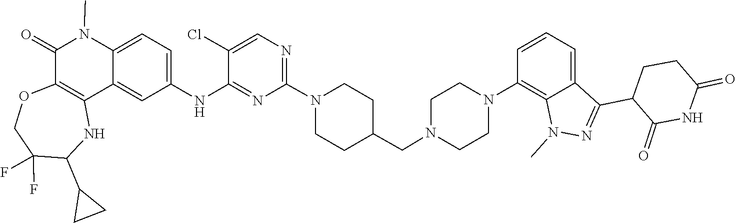 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)C(C7CC7)N6)n5)CC4)CC3)c21] |
| 130a | 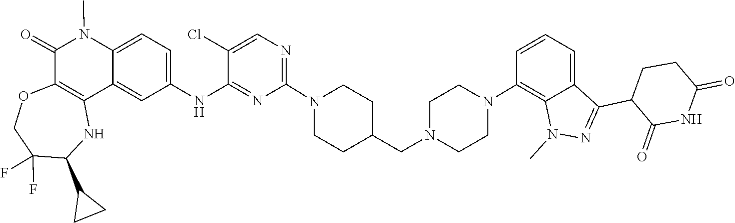 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)[C@H](C7CC7)N6)n5)CC4)CC3)c21] |
| 132 | 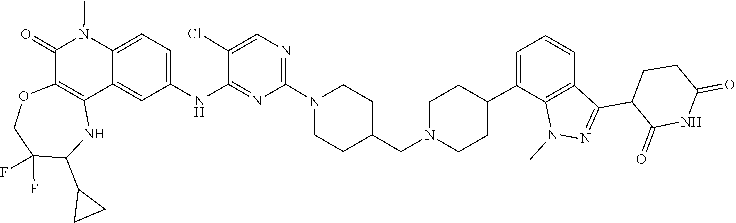 [SMILES: Cn1nc(C2CCC(=O)NC2=O)c2cccc(C3CCN(CC4CCN(c5ncc(Cl)c(Nc6ccc7c(c6)c6c(c(=O)n7C)OCC(F)(F)C(C7CC7)N6)n5)CC4)CC3)c21] |
| 132a | 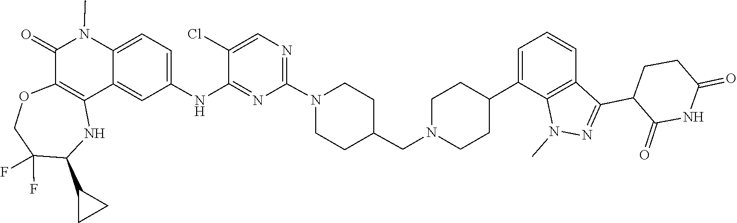 [SMILES: Cn1nc(C2CCC(=O)NC2=O)c2cccc(C3CCN(CC4CCN(c5ncc(Cl)c(Nc6ccc7c(c6)c6c(c(=O)n7C)OCC(F)(F)[C@H](C7CC7)N6)n5)CC4)CC3)c21] |
| 133 | 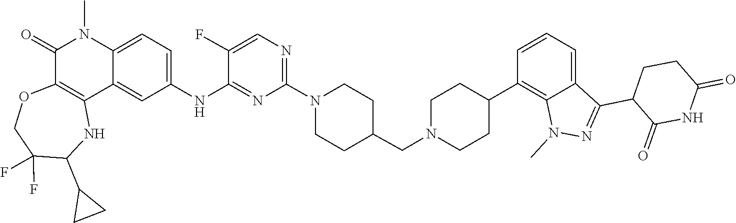 [SMILES: Cn1nc(C2CCC(=O)NC2=O)c2cccc(C3CCN(CC4CCN(c5ncc(F)c(Nc6ccc7c(c6)c6c(c(=O)n7C)OCC(F)(F)C(C7CC7)N6)n5)CC4)CC3)c21] |
| 133a | 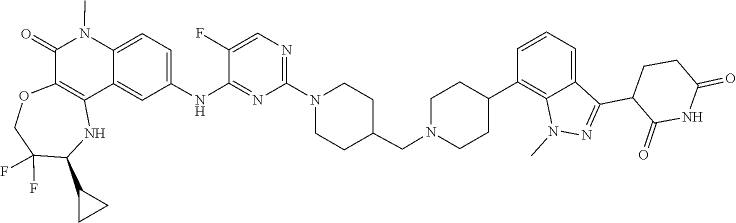 [SMILES: Cn1nc(C2CCC(=O)NC2=O)c2cccc(C3CCN(CC4CCN(c5ncc(F)c(Nc6ccc7c(c6)c6c(c(=O)n7C)OCC(F)(F)[C@H](C7CC7)N6)n5)CC4)CC3)c21] |
| 170 | 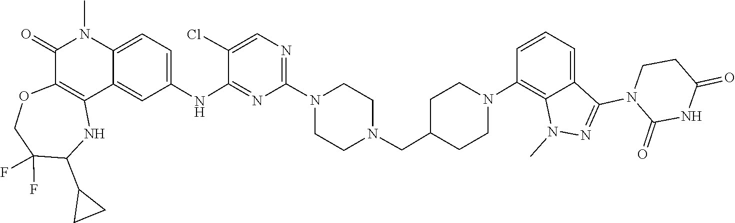 [SMILES: Cn1nc(N2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)CC4)CC3)c21] |
| 170a | 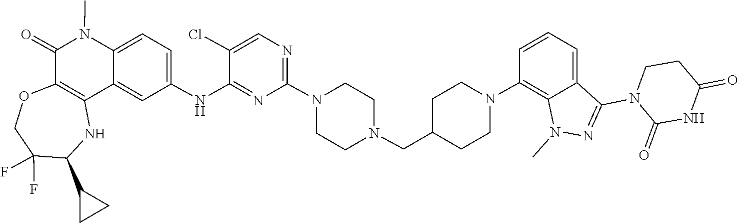 [SMILES: Cn1nc(N2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)CC4)CC3)c21] |
| 186 | 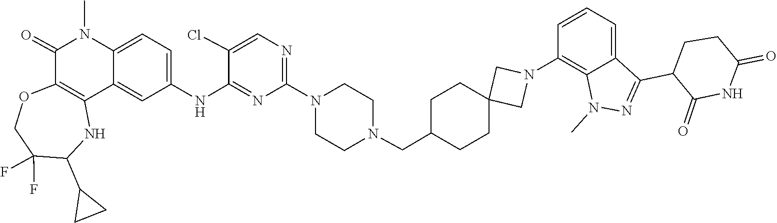 [SMILES: Cn1nc(C2CCC(=O)NC2=O)c2cccc(N3CC4(CCC(CN5CCN(c6ncc(Cl)c(Nc7ccc8c(c7)c7c(c(=O)n8C)OCC(F)(F)C(C8CC8)N7)n6)CC5)CC4)C3)c21] |
| 186a | 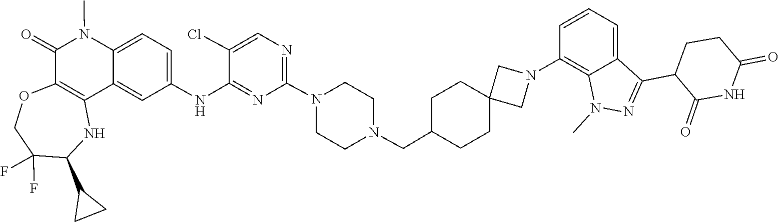 [SMILES: Cn1nc(C2CCC(=O)NC2=O)c2cccc(N3CC4(CCC(CN5CCN(c6ncc(Cl)c(Nc7ccc8c(c7)c7c(c(=O)n8C)OCC(F)(F)[C@H](C8CC8)N7)n6)CC5)CC4)C3)c21] |
| 194 | 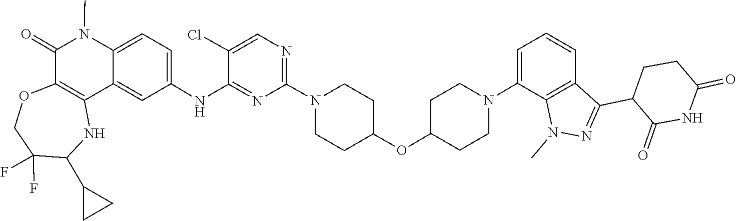 [SMILES: Cn1nc(C2CCC(=O)NC2=O)c2cccc(N3CCC(OC4CCN(c5ncc(Cl)c(Nc6ccc7c(c6)c6c(c(=O)n7C)OCC(F)(F)C(C7CC7)N6)n5)CC4)CC3)c21] |
| 194a | 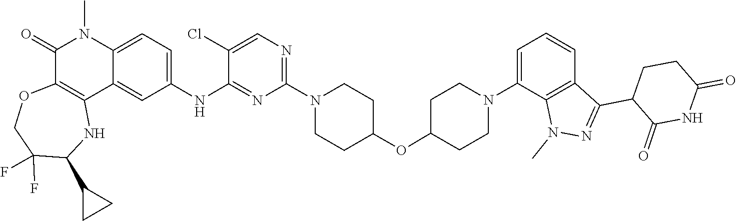 [SMILES: Cn1nc(C2CCC(=O)NC2=O)c2cccc(N3CCC(OC4CCN(c5ncc(Cl)c(Nc6ccc7c(c6)c6c(c(=O)n7C)OCC(F)(F)[C@H](C7CC7)N6)n5)CC4)CC3)c21] |
| 214 | 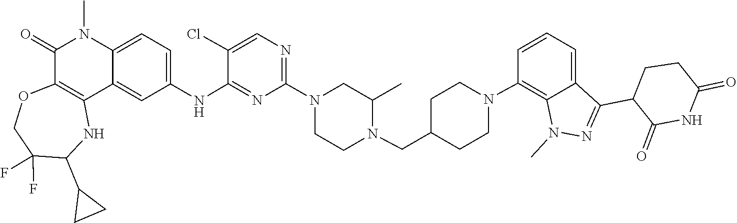 [SMILES: CC1CN(c2ncc(Cl)c(Nc3ccc4c(c3)c3c(c(=O)n4C)OCC(F)(F)C(C4CC4)N3)n2)CCN1CC1CCN(c2cccc3c(C4CCC(=O)NC4=O)nn(C)c23)CC1] |
| 214a | 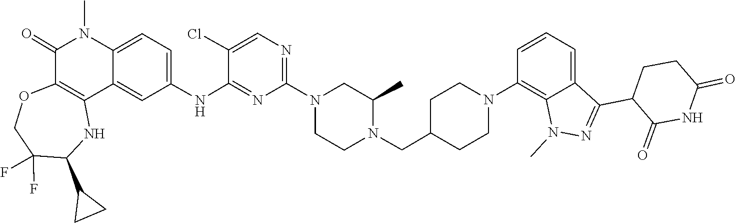 [SMILES: C[C@@H]1CN(c2ncc(Cl)c(Nc3ccc4c(c3)c3c(c(=O)n4C)OCC(F)(F)[C@H](C4CC4)N3)n2)CCN1CC1CCN(c2cccc3c(C4CCC(=O)NC4=O)nn(C)c23)CC1] |
| 214b | 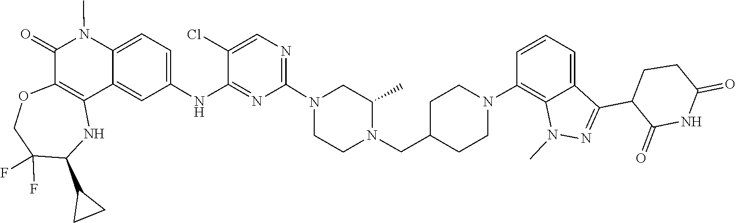 [SMILES: C[C@H]1CN(c2ncc(Cl)c(Nc3ccc4c(c3)c3c(c(=O)n4C)OCC(F)(F)[C@H](C4CC4)N3)n2)CCN1CC1CCN(c2cccc3c(C4CCC(=O)NC4=O)nn(C)c23)CC1] |
| 374 | 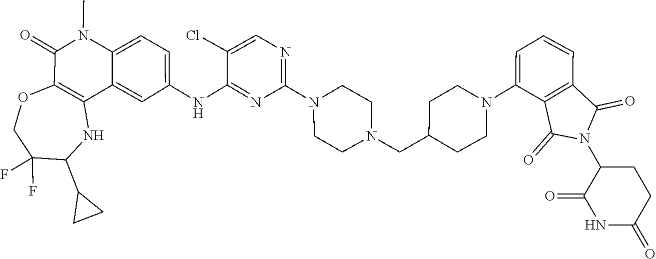 [SMILES: Cn1c(=O)c2c(c3cc(Nc4nc(N5CCN(CC6CCN(c7cccc8c7C(=O)N(C7CCC(=O)NC7=O)C8=O)CC6)CC5)ncc4Cl)ccc31)NC(C1CC1)C(F)(F)CO2] |
| 374a | 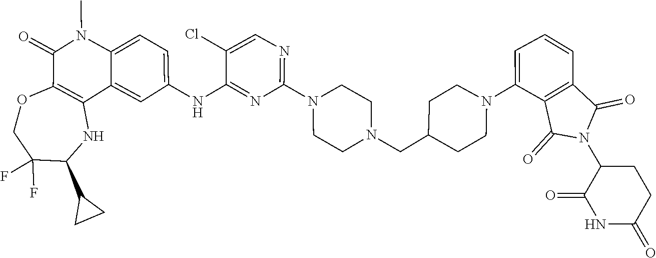 [SMILES: Cn1c(=O)c2c(c3cc(Nc4nc(N5CCN(CC6CCN(c7cccc8c7C(=O)N(C7CCC(=O)NC7=O)C8=O)CC6)CC5)ncc4Cl)ccc31)N[C@@H](C1CC1)C(F)(F)CO2] |
| 375 | 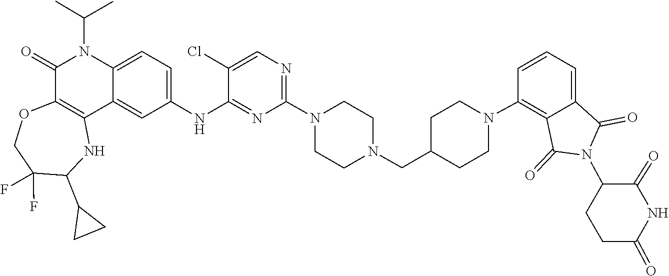 [SMILES: CC(C)n1c(=O)c2c(c3cc(Nc4nc(N5CCN(CC6CCN(c7cccc8c7C(=O)N(C7CCC(=O)NC7=O)C8=O)CC6)CC5)ncc4Cl)ccc31)NC(C1CC1)C(F)(F)CO2] |
| 375a | 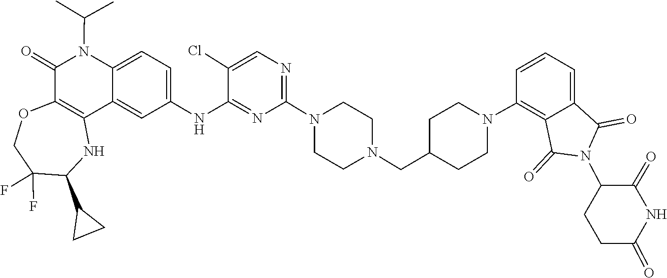 [SMILES: CC(C)n1c(=O)c2c(c3cc(Nc4nc(N5CCN(CC6CCN(c7cccc8c7C(=O)N(C7CCC(=O)NC7=O)C8=O)CC6)CC5)ncc4Cl)ccc31)N[C@@H](C1CC1)C(F)(F)CO2] |
| 379 | 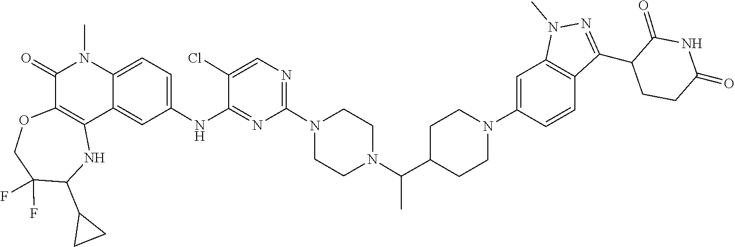 [SMILES: CC(C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)C(C4CC4)N3)n2)CC1] |
| 379a | 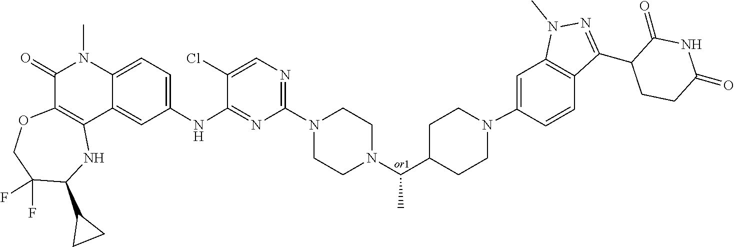 [SMILES: C[C@@H](C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)[C@H](C4CC4)N3)n2)CC1] |
| 379b | 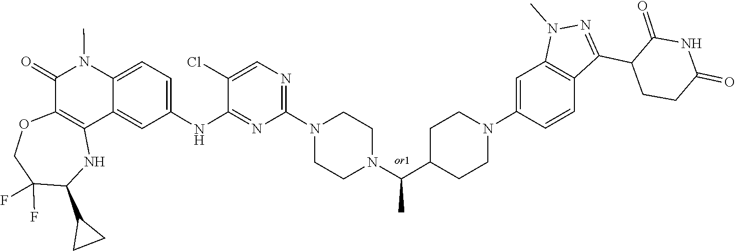 [SMILES: C[C@H](C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)[C@H](C4CC4)N3)n2)CC1] |
| 380 | 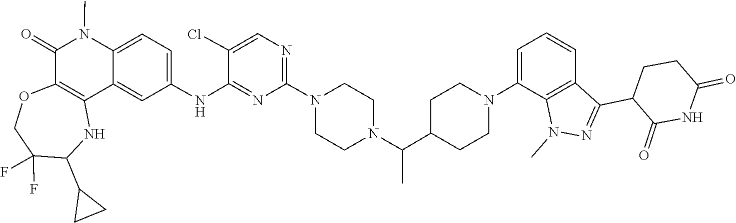 [SMILES: CC(C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)C(C4CC4)N3)n2)CC1] |
| 380a | 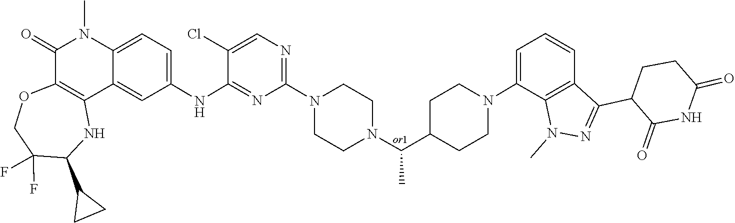 [SMILES: C[C@@H](C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)[C@H](C4CC4)N3)n2)CC1] |
| 380b | 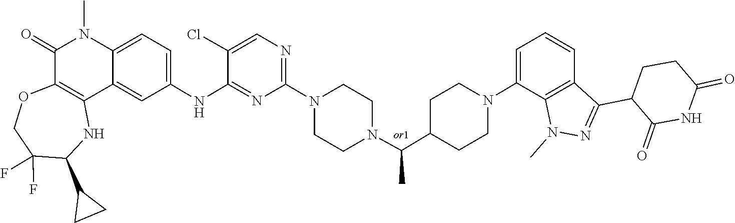 [SMILES: C[C@H](C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)[C@H](C4CC4)N3)n2)CC1] |
| 381 | 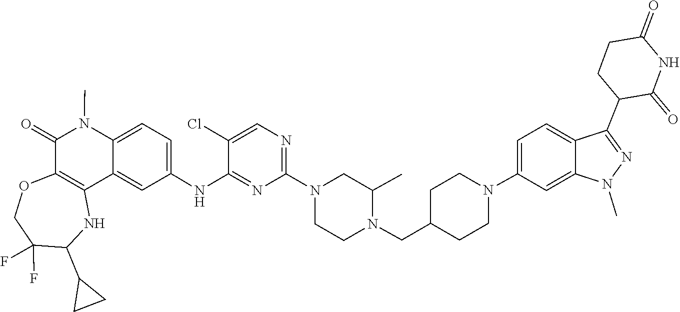 [SMILES: CC1CN(c2ncc(Cl)c(Nc3ccc4c(c3)c3c(c(=O)n4C)OCC(F)(F)C(C4CC4)N3)n2)CCN1CC1CCN(c2ccc3c(C4CCC(=O)NC4=O)nn(C)c3c2)CC1] |
| 381a | 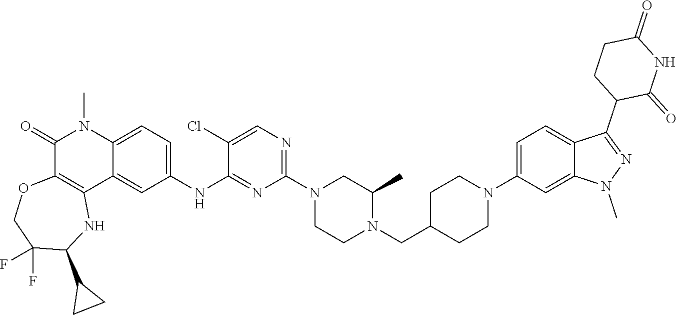 [SMILES: C[C@@H]1CN(c2ncc(Cl)c(Nc3ccc4c(c3)c3c(c(=O)n4C)OCC(F)(F)[C@H](C4CC4)N3)n2)CCN1CC1CCN(c2ccc3c(C4CCC(=O)NC4=O)nn(C)c3c2)CC1] |
| 381b | 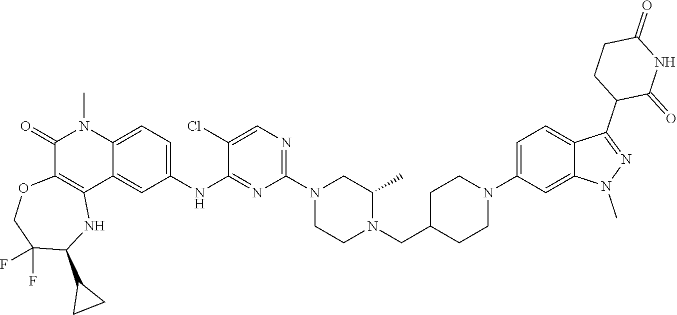 [SMILES: C[C@H]1CN(c2ncc(Cl)c(Nc3ccc4c(c3)c3c(c(=O)n4C)OCC(F)(F)[C@H](C4CC4)N3)n2)CCN1CC1CCN(c2ccc3c(C4CCC(=O)NC4=O)nn(C)c3c2)CC1] |
| 382 | 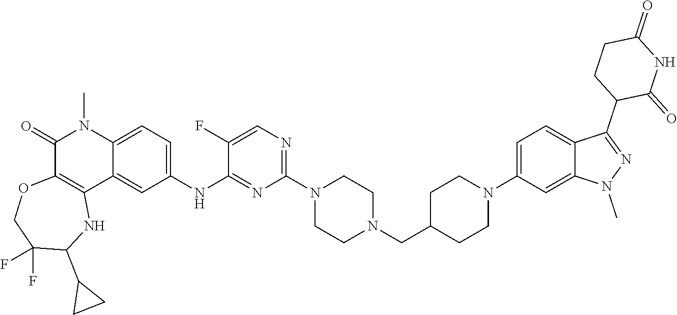 [SMILES: Cn1nc(C2CCC(=O)NC2=O)c2ccc(N3CCC(CN4CCN(c5ncc(F)c(Nc6ccc7c(c6)c6c(c(=O)n7C)OCC(F)(F)C(C7CC7)N6)n5)CC4)CC3)cc21] |
| 382a | 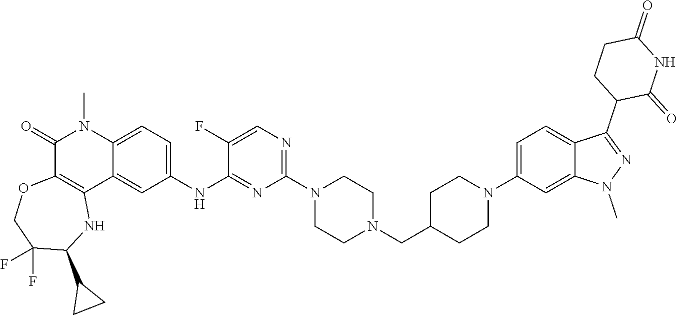 [SMILES: Cn1nc(C2CCC(=O)NC2=O)c2ccc(N3CCC(CN4CCN(c5ncc(F)c(Nc6ccc7c(c6)c6c(c(=O)n7C)OCC(F)(F)[C@H](C7CC7)N6)n5)CC4)CC3)cc21] |
| 383 | 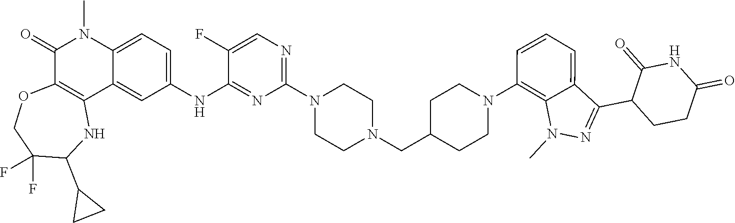 [SMILES: Cn1nc(C2CCC(=O)NC2=O)c2cccc(N3CCC(CN4CCN(c5ncc(F)c(Nc6ccc7c(c6)c6c(c(=O)n7C)OCC(F)(F)C(C7CC7)N6)n5)CC4)CC3)c21] |
| 383a | 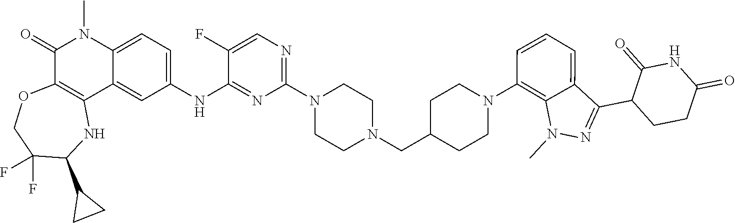 [SMILES: Cn1nc(C2CCC(=O)NC2=O)c2cccc(N3CCC(CN4CCN(c5ncc(F)c(Nc6ccc7c(c6)c6c(c(=O)n7C)OCC(F)(F)[C@H](C7CC7)N6)n5)CC4)CC3)c21] |
| 384 | 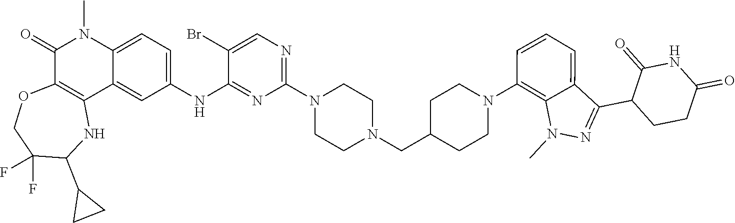 [SMILES: Cn1nc(C2CCC(=O)NC2=O)c2cccc(N3CCC(CN4CCN(c5ncc(Br)c(Nc6ccc7c(c6)c6c(c(=O)n7C)OCC(F)(F)C(C7CC7)N6)n5)CC4)CC3)c21] |
| 384a | 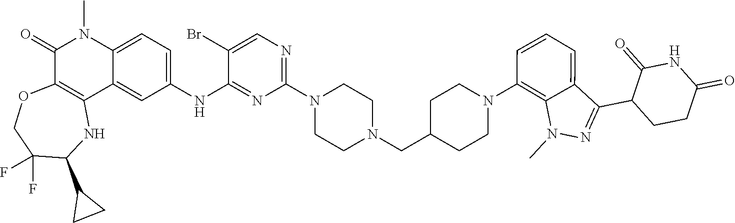 [SMILES: Cn1nc(C2CCC(=O)NC2=O)c2cccc(N3CCC(CN4CCN(c5ncc(Br)c(Nc6ccc7c(c6)c6c(c(=O)n7C)OCC(F)(F)[C@H](C7CC7)N6)n5)CC4)CC3)c21] |
| 385 | 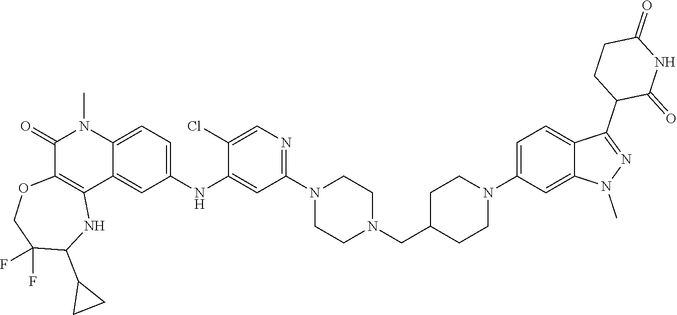 [SMILES: Cn1nc(C2CCC(=O)NC2=O)c2ccc(N3CCC(CN4CCN(c5cc(Nc6ccc7c(c6)c6c(c(=O)n7C)OCC(F)(F)C(C7CC7)N6)c(Cl)cn5)CC4)CC3)cc21] |
| 385a | 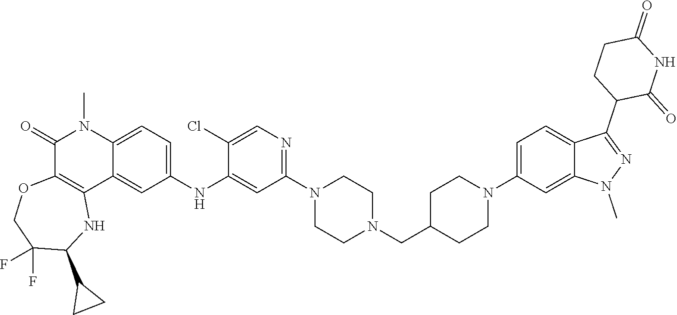 [SMILES: Cn1nc(C2CCC(=O)NC2=O)c2ccc(N3CCC(CN4CCN(c5cc(Nc6ccc7c(c6)c6c(c(=O)n7C)OCC(F)(F)[C@H](C7CC7)N6)c(Cl)cn5)CC4)CC3)cc21] |
| 386 | 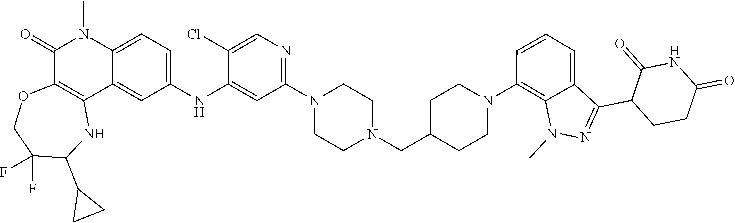 [SMILES: Cn1nc(C2CCC(=O)NC2=O)c2cccc(N3CCC(CN4CCN(c5cc(Nc6ccc7c(c6)c6c(c(=O)n7C)OCC(F)(F)C(C7CC7)N6)c(Cl)cn5)CC4)CC3)c21] |
| 386a | 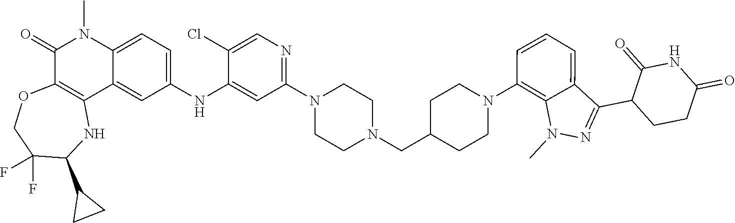 [SMILES: Cn1nc(C2CCC(=O)NC2=O)c2cccc(N3CCC(CN4CCN(c5cc(Nc6ccc7c(c6)c6c(c(=O)n7C)OCC(F)(F)[C@H](C7CC7)N6)c(Cl)cn5)CC4)CC3)c21] |
| 387 | 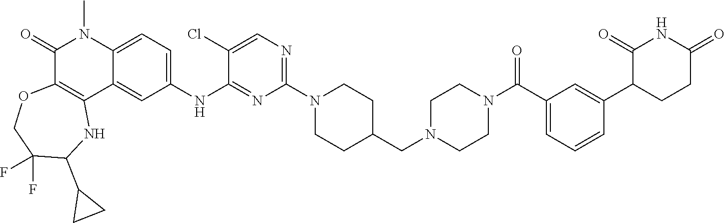 [SMILES: Cn1c(=O)c2c(c3cc(Nc4nc(N5CCC(CN6CCN(C(=O)c7cccc(C8CCC(=O)NC8=O)c7)CC6)CC5)ncc4Cl)ccc31)NC(C1CC1)C(F)(F)CO2] |
| 387a | 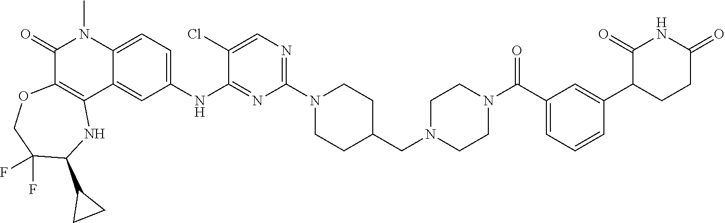 [SMILES: Cn1c(=O)c2c(c3cc(Nc4nc(N5CCC(CN6CCN(C(=O)c7cccc(C8CCC(=O)NC8=O)c7)CC6)CC5)ncc4Cl)ccc31)N[C@@H](C1CC1)C(F)(F)CO2] |
| 388 | 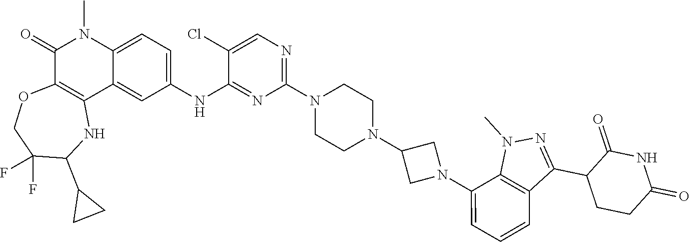 [SMILES: Cn1nc(C2CCC(=O)NC2=O)c2cccc(N3CC(N4CCN(c5ncc(Cl)c(Nc6ccc7c(c6)c6c(c(=O)n7C)OCC(F)(F)C(C7CC7)N6)n5)CC4)C3)c21] |
| 388a | 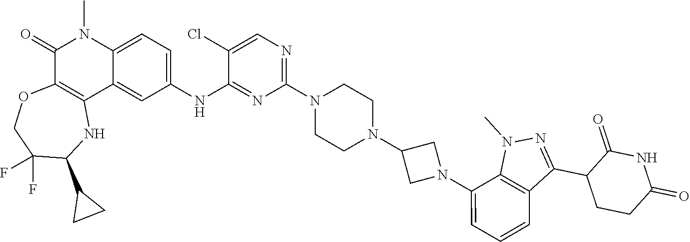 [SMILES: Cn1nc(C2CCC(=O)NC2=O)c2cccc(N3CC(N4CCN(c5ncc(Cl)c(Nc6ccc7c(c6)c6c(c(=O)n7C)OCC(F)(F)[C@H](C7CC7)N6)n5)CC4)C3)c21] |
| 389 | 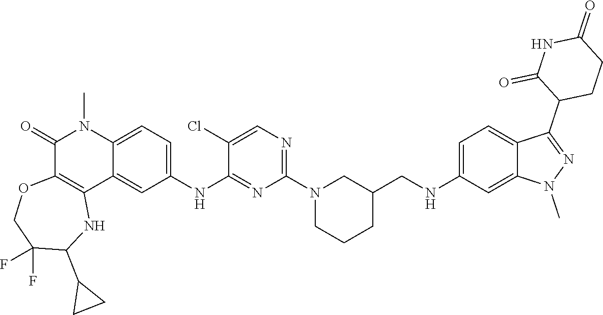 [SMILES: Cn1nc(C2CCC(=O)NC2=O)c2ccc(NCC3CCCN(c4ncc(Cl)c(Nc5ccc6c(c5)c5c(c(=O)n6C)OCC(F)(F)C(C6CC6)N5)n4)C3)cc21] |
| 389a | 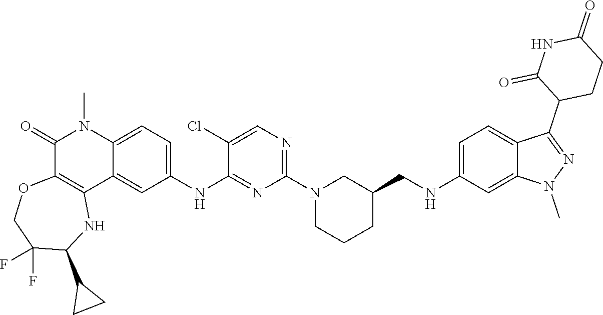 [SMILES: Cn1nc(C2CCC(=O)NC2=O)c2ccc(NC[C@H]3CCCN(c4ncc(Cl)c(Nc5ccc6c(c5)c5c(c(=O)n6C)OCC(F)(F)[C@H](C6CC6)N5)n4)C3)cc21] |
| 390 | 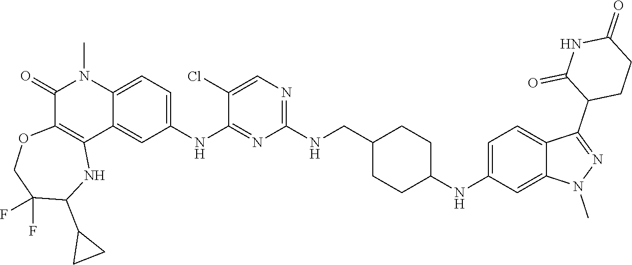 [SMILES: Cn1nc(C2CCC(=O)NC2=O)c2ccc(NC3CCC(CNc4ncc(Cl)c(Nc5ccc6c(c5)c5c(c(=O)n6C)OCC(F)(F)C(C6CC6)N5)n4)CC3)cc21] |
| 390a | 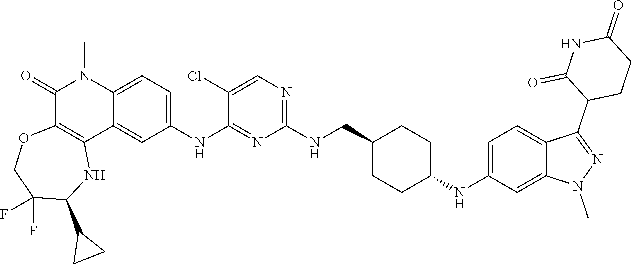 [SMILES: Cn1nc(C2CCC(=O)NC2=O)c2ccc(N[C@H]3CC[C@H](CNc4ncc(Cl)c(Nc5ccc6c(c5)c5c(c(=O)n6C)OCC(F)(F)[C@H](C6CC6)N5)n4)CC3)cc21] |
| 391 | 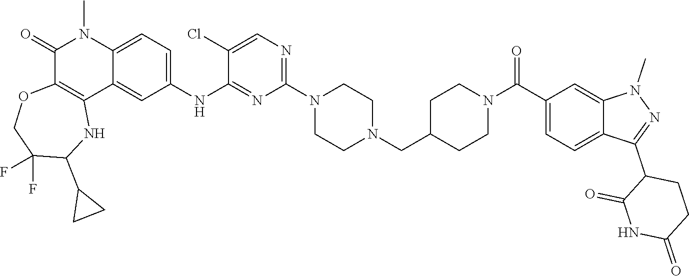 [SMILES: Cn1nc(C2CCC(=O)NC2=O)c2ccc(C(=O)N3CCC(CN4CCN(c5ncc(Cl)c(Nc6ccc7c(c6)c6c(c(=O)n7C)OCC(F)(F)C(C7CC7)N6)n5)CC4)CC3)cc21] |
| 391a | 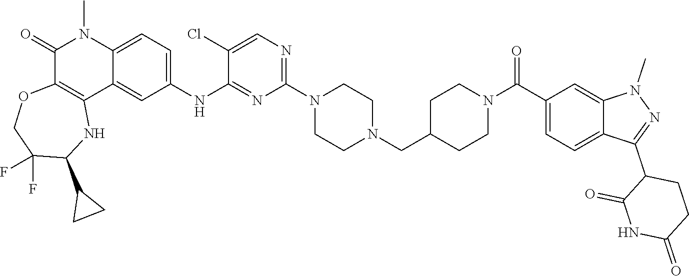 [SMILES: Cn1nc(C2CCC(=O)NC2=O)c2ccc(C(=O)N3CCC(CN4CCN(c5ncc(Cl)c(Nc6ccc7c(c6)c6c(c(=O)n7C)OCC(F)(F)[C@H](C7CC7)N6)n5)CC4)CC3)cc21] |
| 392 | 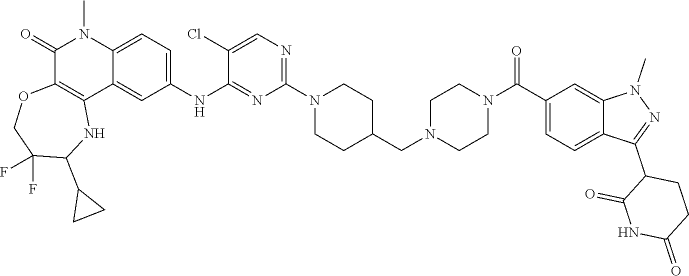 [SMILES: Cn1nc(C2CCC(=O)NC2=O)c2ccc(C(=O)N3CCN(CC4CCN(c5ncc(Cl)c(Nc6ccc7c(c6)c6c(c(=O)n7C)OCC(F)(F)C(C7CC7)N6)n5)CC4)CC3)cc21] |
| 392a | 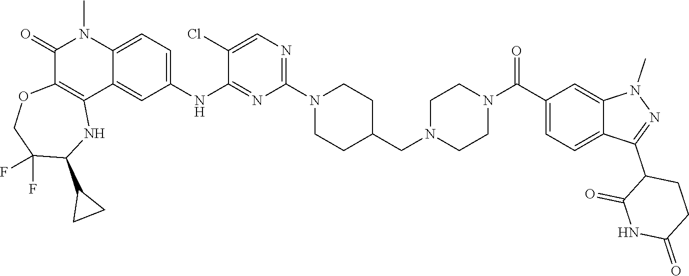 [SMILES: Cn1nc(C2CCC(=O)NC2=O)c2ccc(C(=O)N3CCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 393 | 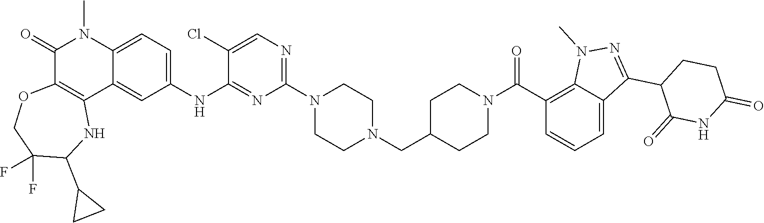 [SMILES: Cn1nc(C2CCC(=O)NC2=O)c2cccc(C(=O)N3CCC(CN4CCN(c5ncc(Cl)c(Nc6ccc7c(c6)c6c(c(=O)n7C)OCC(F)(F)C(C7CC7)N6)n5)CC4)CC3)c21] |
| 393a | 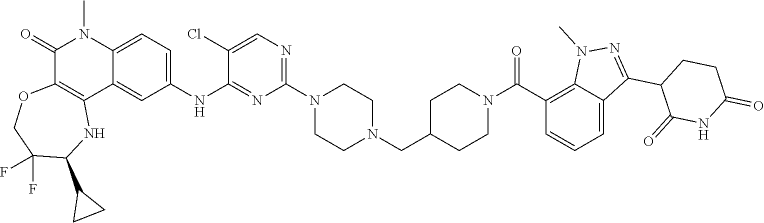 [SMILES: Cn1nc(C2CCC(=O)NC2=O)c2cccc(C(=O)N3CCC(CN4CCN(c5ncc(Cl)c(Nc6ccc7c(c6)c6c(c(=O)n7C)OCC(F)(F)[C@H](C7CC7)N6)n5)CC4)CC3)c21] |
| 394 | 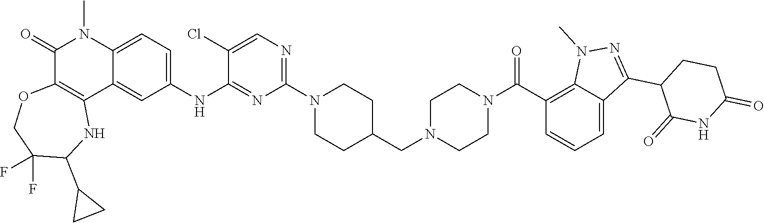 [SMILES: Cn1nc(C2CCC(=O)NC2=O)c2cccc(C(=O)N3CCN(CC4CCN(c5ncc(Cl)c(Nc6ccc7c(c6)c6c(c(=O)n7C)OCC(F)(F)C(C7CC7)N6)n5)CC4)CC3)c21] |
| 394a | 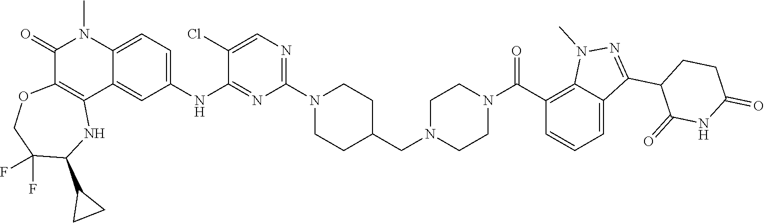 [SMILES: Cn1nc(C2CCC(=O)NC2=O)c2cccc(C(=O)N3CCN(CC4CCN(c5ncc(Cl)c(Nc6ccc7c(c6)c6c(c(=O)n7C)OCC(F)(F)[C@H](C7CC7)N6)n5)CC4)CC3)c21] |
| 395 | 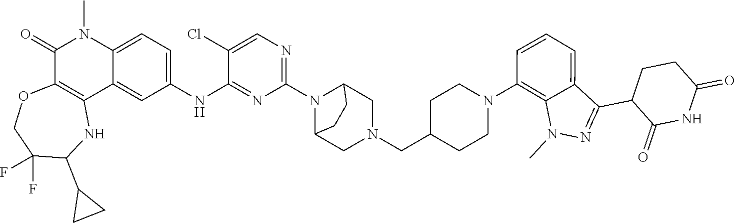 [SMILES: Cn1nc(C2CCC(=O)NC2=O)c2cccc(N3CCC(CN4CC5CCC(C4)N5c4ncc(Cl)c(Nc5ccc6c(c5)c5c(c(=O)n6C)OCC(F)(F)C(C6CC6)N5)n4)CC3)c21] |
| 395a | 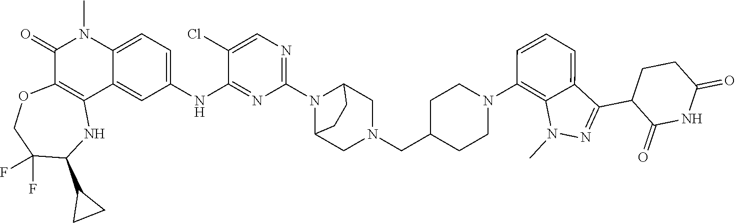 [SMILES: Cn1nc(C2CCC(=O)NC2=O)c2cccc(N3CCC(CN4CC5CCC(C4)N5c4ncc(Cl)c(Nc5ccc6c(c5)c5c(c(=O)n6C)OCC(F)(F)[C@H](C6CC6)N5)n4)CC3)c21] |
| 396 | 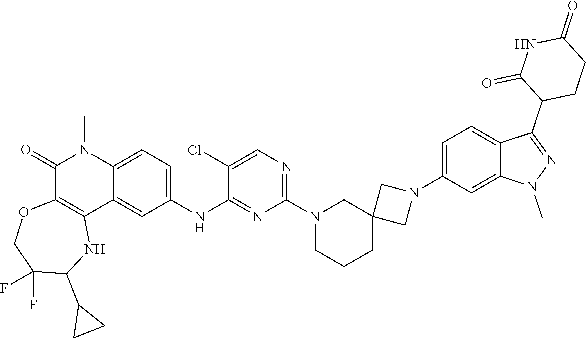 [SMILES: Cn1nc(C2CCC(=O)NC2=O)c2ccc(N3CC4(CCCN(c5ncc(Cl)c(Nc6ccc7c(c6)c6c(c(=O)n7C)OCC(F)(F)C(C7CC7)N6)n5)C4)C3)cc21] |
| 396a | 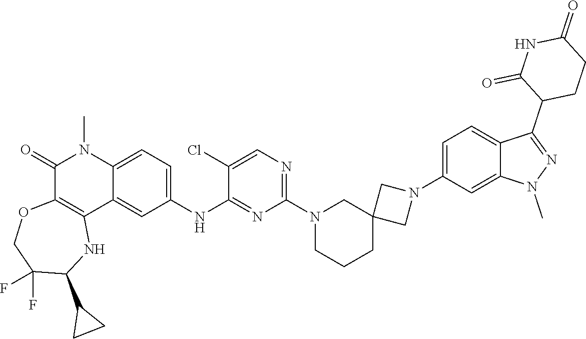 [SMILES: Cn1nc(C2CCC(=O)NC2=O)c2ccc(N3CC4(CCCN(c5ncc(Cl)c(Nc6ccc7c(c6)c6c(c(=O)n7C)OCC(F)(F)[C@H](C7CC7)N6)n5)C4)C3)cc21] |
| 397 | 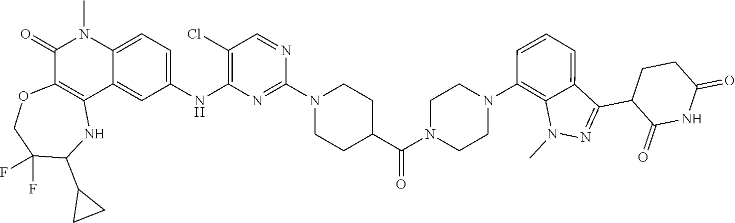 [SMILES: Cn1nc(C2CCC(=O)NC2=O)c2cccc(N3CCN(C(=O)C4CCN(c5ncc(Cl)c(Nc6ccc7c(c6)c6c(c(=O)n7C)OCC(F)(F)C(C7CC7)N6)n5)CC4)CC3)c21] |
| 397a | 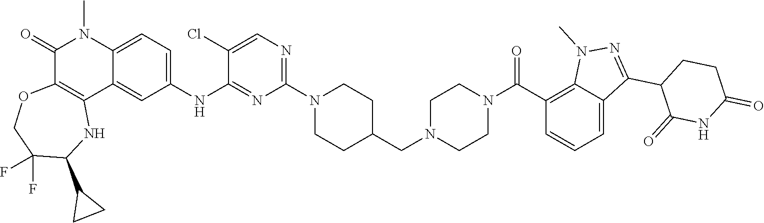 [SMILES: Cn1nc(C2CCC(=O)NC2=O)c2cccc(C(=O)N3CCN(CC4CCN(c5ncc(Cl)c(Nc6ccc7c(c6)c6c(c(=O)n7C)OCC(F)(F)[C@H](C7CC7)N6)n5)CC4)CC3)c21] |
| 398 | 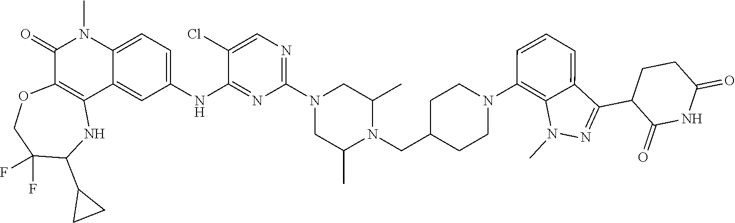 [SMILES: CC1CN(c2ncc(Cl)c(Nc3ccc4c(c3)c3c(c(=O)n4C)OCC(F)(F)C(C4CC4)N3)n2)CC(C)N1CC1CCN(c2cccc3c(C4CCC(=O)NC4=O)nn(C)c23)CC1] |
| 398a | 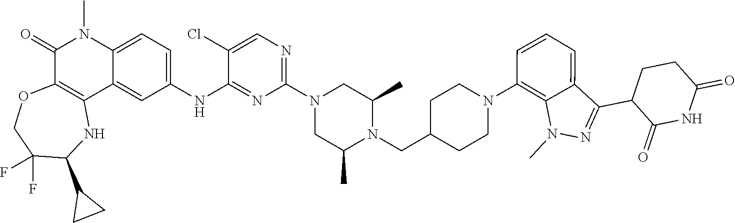 [SMILES: C[C@@H]1CN(c2ncc(Cl)c(Nc3ccc4c(c3)c3c(c(=O)n4C)OCC(F)(F)[C@H](C4CC4)N3)n2)C[C@H](C)N1CC1CCN(c2cccc3c(C4CCC(=O)NC4=O)nn(C)c23)CC1] |
| 399 | 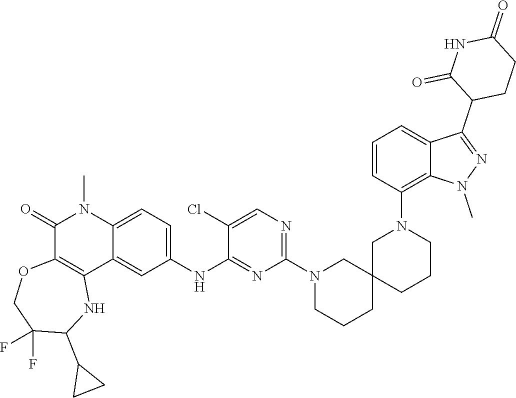 [SMILES: Cn1nc(C2CCC(=O)NC2=O)c2cccc(N3CCCC4(CCCN(c5ncc(Cl)c(Nc6ccc7c(c6)c6c(c(=O)n7C)OCC(F)(F)C(C7CC7)N6)n5)C4)C3)c21] |
| 399a | 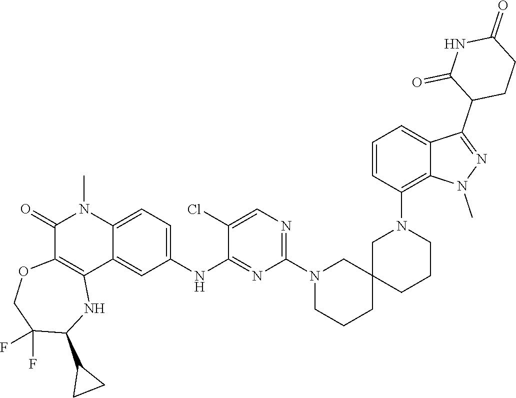 [SMILES: Cn1nc(C2CCC(=O)NC2=O)c2cccc(N3CCCC4(CCCN(c5ncc(Cl)c(Nc6ccc7c(c6)c6c(c(=O)n7C)OCC(F)(F)[C@H](C7CC7)N6)n5)C4)C3)c21] |
| 400 | 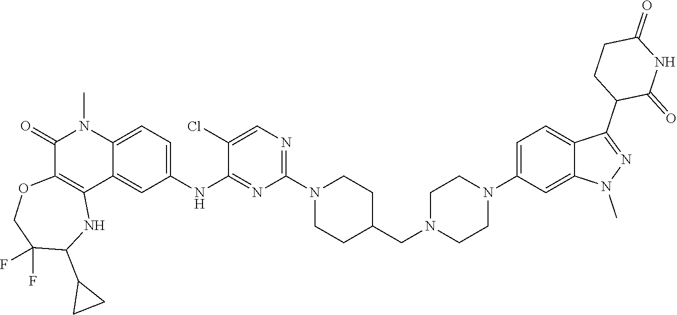 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)CC4)CC3)cc21] |
| 400a | 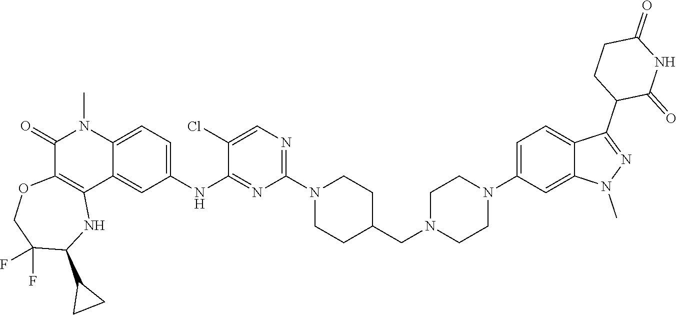 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 401 | 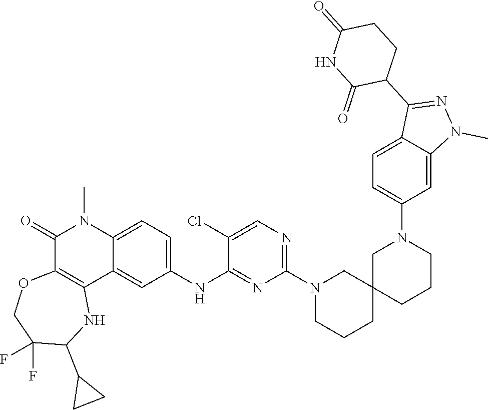 [SMILES: Cn1nc(C2CCC(=O)NC2=O)c2ccc(N3CCCC4(CCCN(c5ncc(Cl)c(Nc6ccc7c(c6)c6c(c(=O)n7C)OCC(F)(F)C(C7CC7)N6)n5)C4)C3)cc21] |
| 401a | 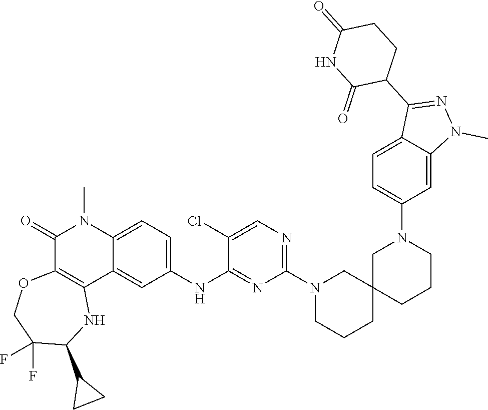 [SMILES: Cn1nc(C2CCC(=O)NC2=O)c2ccc(N3CCCC4(CCCN(c5ncc(Cl)c(Nc6ccc7c(c6)c6c(c(=O)n7C)OCC(F)(F)[C@H](C7CC7)N6)n5)C4)C3)cc21] |
| 402 | 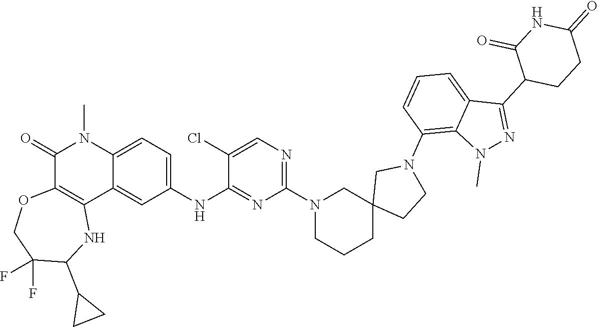 [SMILES: Cn1nc(C2CCC(=O)NC2=O)c2cccc(N3CCC4(CCCN(c5ncc(Cl)c(Nc6ccc7c(c6)c6c(c(=O)n7C)OCC(F)(F)C(C7CC7)N6)n5)C4)C3)c21] |
| 402a | 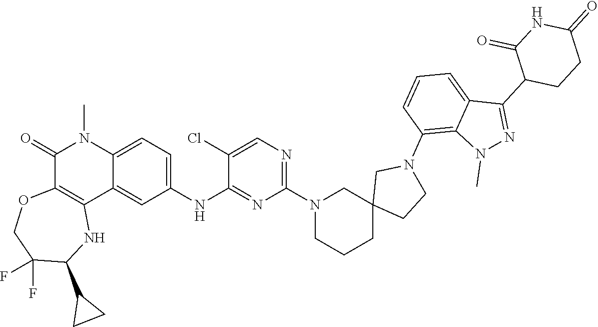 [SMILES: Cn1nc(C2CCC(=O)NC2=O)c2cccc(N3CCC4(CCCN(c5ncc(Cl)c(Nc6ccc7c(c6)c6c(c(=O)n7C)OCC(F)(F)[C@H](C7CC7)N6)n5)C4)C3)c21] |
| 403 | 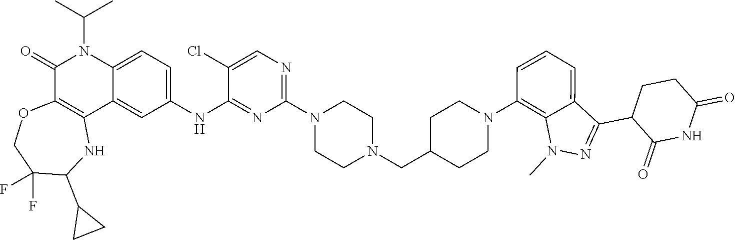 [SMILES: CC(C)n1c(=O)c2c(c3cc(Nc4nc(N5CCN(CC6CCN(c7cccc8c(C9CCC(=O)NC9=O)nn(C)c78)CC6)CC5)ncc4Cl)ccc31)NC(C1CC1)C(F)(F)CO2] |
| 403a | 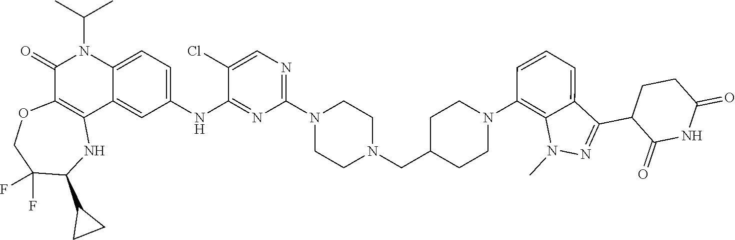 [SMILES: CC(C)n1c(=O)c2c(c3cc(Nc4nc(N5CCN(CC6CCN(c7cccc8c(C9CCC(=O)NC9=O)nn(C)c78)CC6)CC5)ncc4Cl)ccc31)N[C@@H](C1CC1)C(F)(F)CO2] |
| 404 | 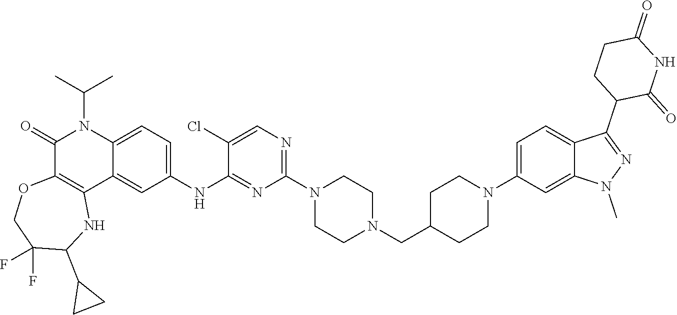 [SMILES: CC(C)n1c(=O)c2c(c3cc(Nc4nc(N5CCN(CC6CCN(c7ccc8c(C9CCC(=O)NC9=O)nn(C)c8c7)CC6)CC5)ncc4Cl)ccc31)NC(C1CC1)C(F)(F)CO2] |
| 404a | 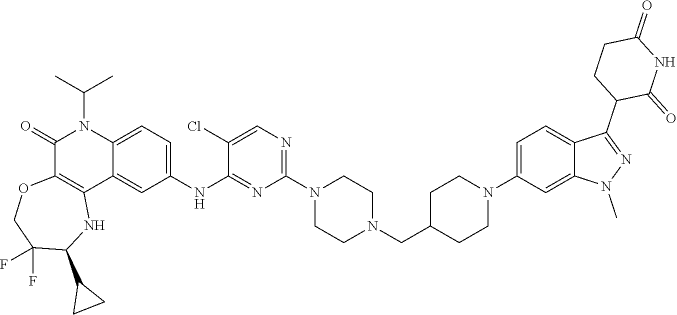 [SMILES: CC(C)n1c(=O)c2c(c3cc(Nc4nc(N5CCN(CC6CCN(c7ccc8c(C9CCC(=O)NC9=O)nn(C)c8c7)CC6)CC5)ncc4Cl)ccc31)N[C@@H](C1CC1)C(F)(F)CO2] |
| 405 | 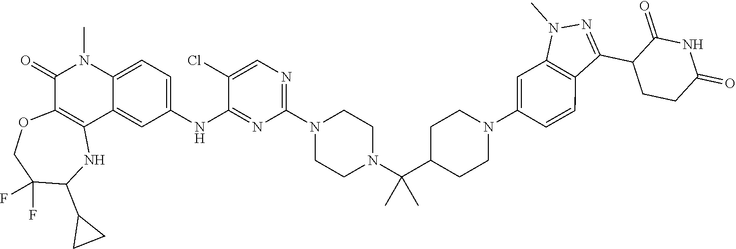 [SMILES: Cn1nc(C2CCC(=O)NC2=O)c2ccc(N3CCC(C(C)(C)N4CCN(c5ncc(Cl)c(Nc6ccc7c(c6)c6c(c(=O)n7C)OCC(F)(F)C(C7CC7)N6)n5)CC4)CC3)cc21] |
| 405a | 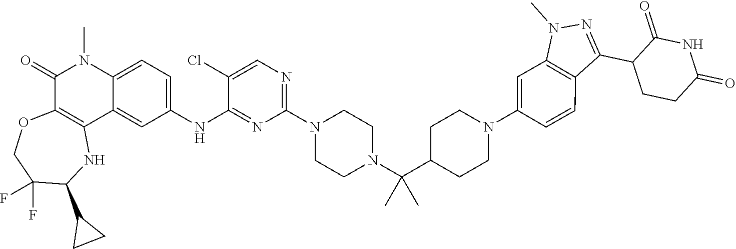 [SMILES: Cn1nc(C2CCC(=O)NC2=O)c2ccc(N3CCC(C(C)(C)N4CCN(c5ncc(Cl)c(Nc6ccc7c(c6)c6c(c(=O)n7C)OCC(F)(F)[C@H](C7CC7)N6)n5)CC4)CC3)cc21] |
| 406 | 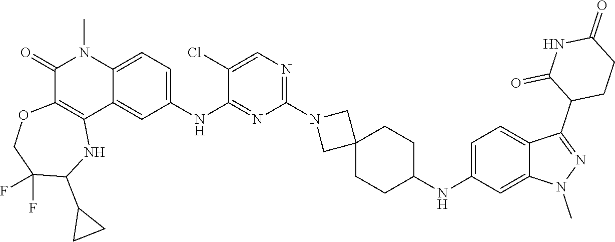 [SMILES: Cn1nc(C2CCC(=O)NC2=O)c2ccc(NC3CCC4(CC3)CN(c3ncc(Cl)c(Nc5ccc6c(c5)c5c(c(=O)n6C)OCC(F)(F)C(C6CC6)N5)n3)C4)cc21] |
| 406a | 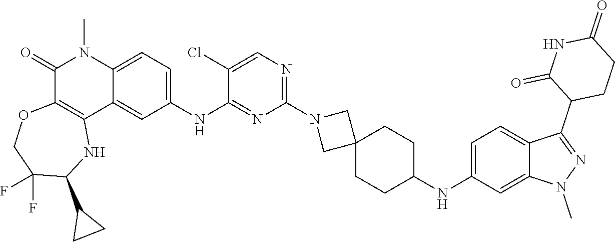 [SMILES: Cn1nc(C2CCC(=O)NC2=O)c2ccc(NC3CCC4(CC3)CN(c3ncc(Cl)c(Nc5ccc6c(c5)c5c(c(=O)n6C)OCC(F)(F)[C@H](C6CC6)N5)n3)C4)cc21] |
| 407 | 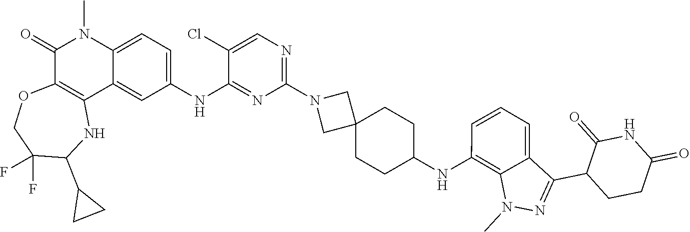 [SMILES: Cn1nc(C2CCC(=O)NC2=O)c2cccc(NC3CCC4(CC3)CN(c3ncc(Cl)c(Nc5ccc6c(c5)c5c(c(=O)n6C)OCC(F)(F)C(C6CC6)N5)n3)C4)c21] |
| 407a | 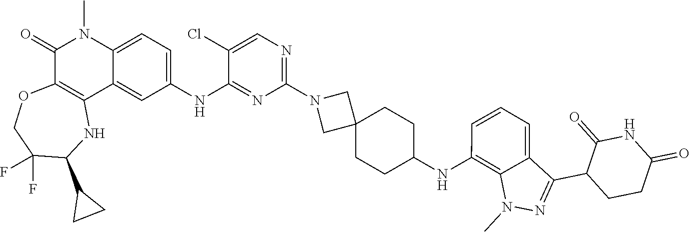 [SMILES: Cn1nc(C2CCC(=O)NC2=O)c2cccc(NC3CCC4(CC3)CN(c3ncc(Cl)c(Nc5ccc6c(c5)c5c(c(=O)n6C)OCC(F)(F)[C@H](C6CC6)N5)n3)C4)c21] |
| 408 | 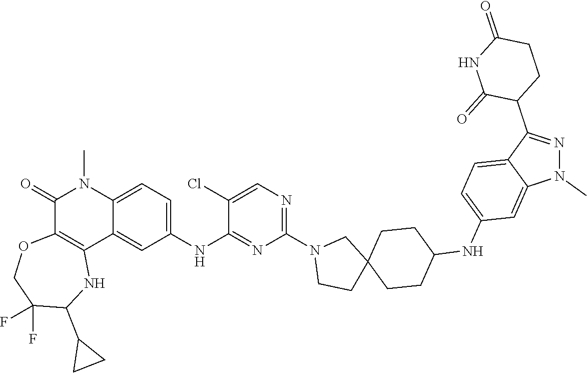 [SMILES: Cn1nc(C2CCC(=O)NC2=O)c2ccc(NC3CCC4(CC3)CCN(c3ncc(Cl)c(Nc5ccc6c(c5)c5c(c(=O)n6C)OCC(F)(F)C(C6CC6)N5)n3)C4)cc21] |
| 408a | 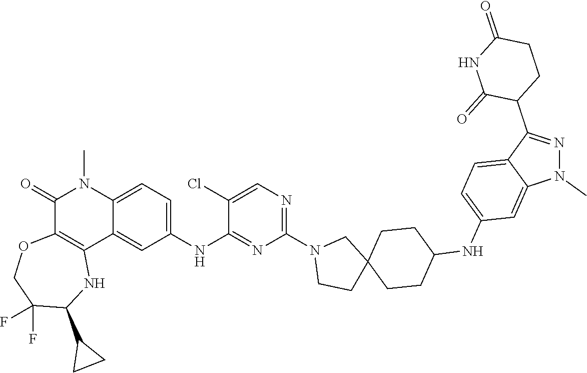 [SMILES: Cn1nc(C2CCC(=O)NC2=O)c2ccc(NC3CCC4(CC3)CCN(c3ncc(Cl)c(Nc5ccc6c(c5)c5c(c(=O)n6C)OCC(F)(F)[C@H](C6CC6)N5)n3)C4)cc21] |
| 409 | 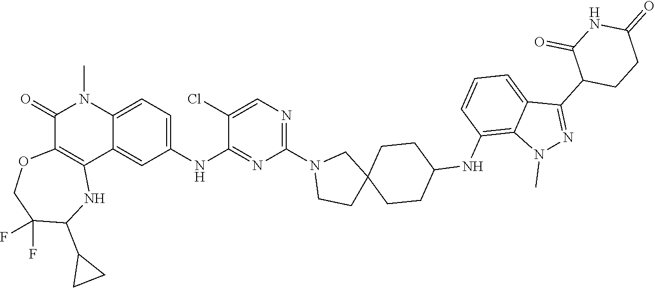 [SMILES: Cn1nc(C2CCC(=O)NC2=O)c2cccc(NC3CCC4(CC3)CCN(c3ncc(Cl)c(Nc5ccc6c(c5)c5c(c(=O)n6C)OCC(F)(F)C(C6CC6)N5)n3)C4)c21] |
| 409a | 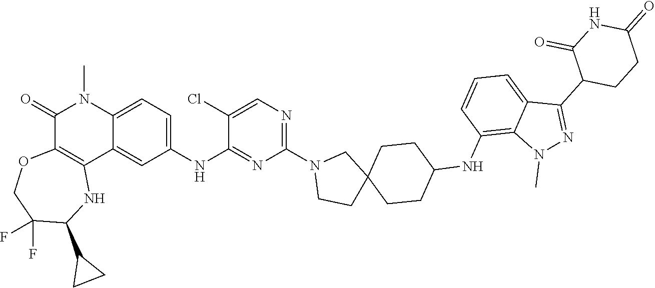 [SMILES: Cn1nc(C2CCC(=O)NC2=O)c2cccc(NC3CCC4(CC3)CCN(c3ncc(Cl)c(Nc5ccc6c(c5)c5c(c(=O)n6C)OCC(F)(F)[C@H](C6CC6)N5)n3)C4)c21] |
| 410 | 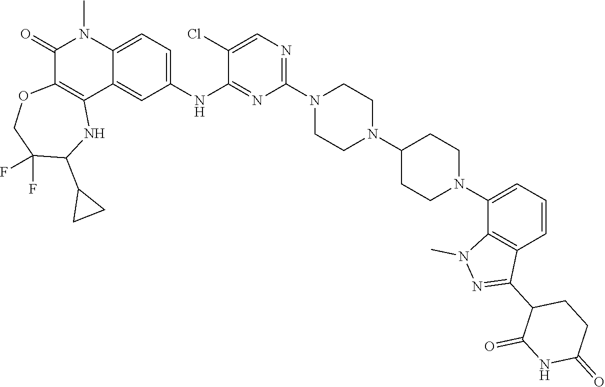 [SMILES: Cn1nc(C2CCC(=O)NC2=O)c2cccc(N3CCC(N4CCN(c5ncc(Cl)c(Nc6ccc7c(c6)c6c(c(=O)n7C)OCC(F)(F)C(C7CC7)N6)n5)CC4)CC3)c21] |
| 410a | 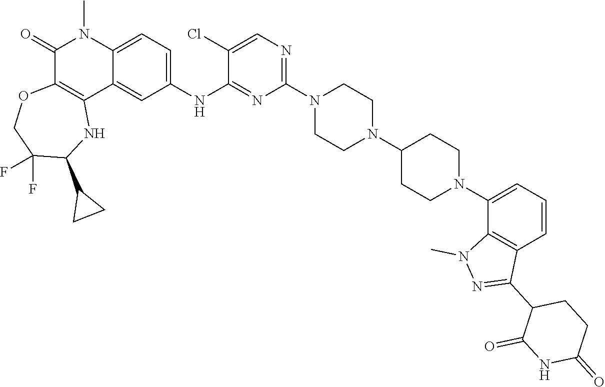 [SMILES: Cn1nc(C2CCC(=O)NC2=O)c2cccc(N3CCC(N4CCN(c5ncc(Cl)c(Nc6ccc7c(c6)c6c(c(=O)n7C)OCC(F)(F)[C@H](C7CC7)N6)n5)CC4)CC3)c21] |
| 411 | 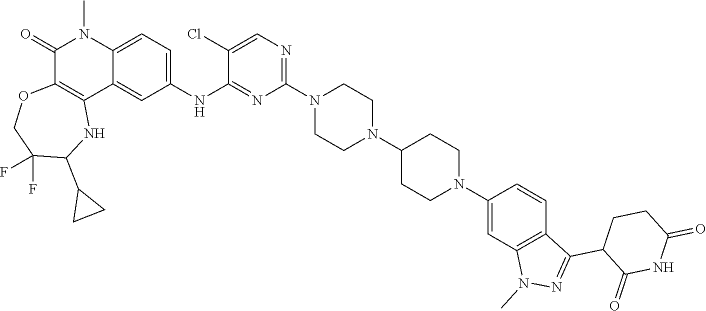 [SMILES: Cn1nc(C2CCC(=O)NC2=O)c2ccc(N3CCC(N4CCN(c5ncc(Cl)c(Nc6ccc7c(c6)c6c(c(=O)n7C)OCC(F)(F)C(C7CC7)N6)n5)CC4)CC3)cc21] |
| 411a | 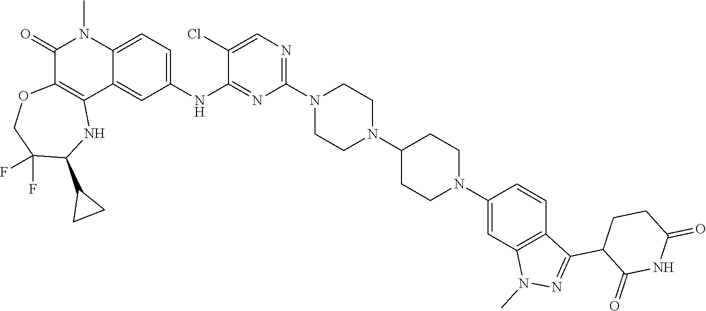 [SMILES: Cn1nc(C2CCC(=O)NC2=O)c2ccc(N3CCC(N4CCN(c5ncc(Cl)c(Nc6ccc7c(c6)c6c(c(=O)n7C)OCC(F)(F)[C@H](C7CC7)N6)n5)CC4)CC3)cc21] |
| 412 | 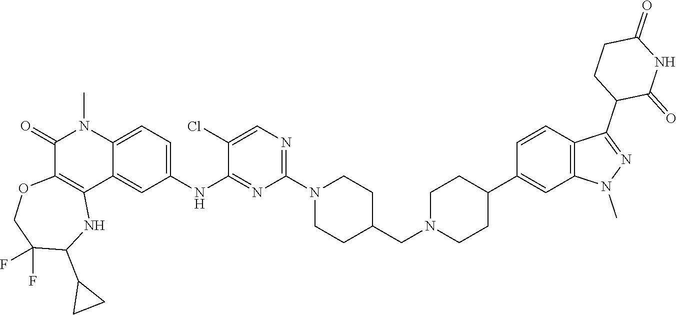 [SMILES: Cn1nc(C2CCC(=O)NC2=O)c2ccc(C3CCN(CC4CCN(c5ncc(Cl)c(Nc6ccc7c(c6)c6c(c(=O)n7C)OCC(F)(F)C(C7CC7)N6)n5)CC4)CC3)cc21] |
| 412a | 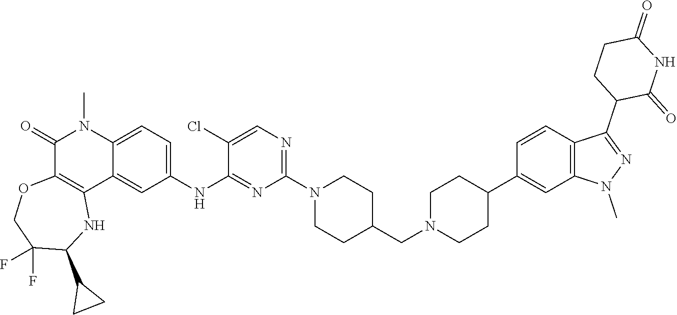 [SMILES: Cn1nc(C2CCC(=O)NC2=O)c2ccc(C3CCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 413 | 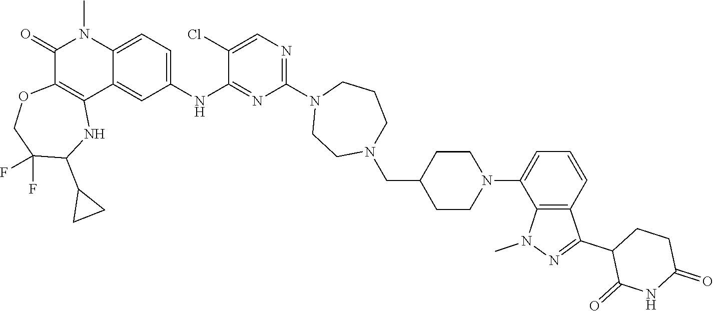 [SMILES: Cn1nc(C2CCC(=O)NC2=O)c2cccc(N3CCC(CN4CCCN(c5ncc(Cl)c(Nc6ccc7c(c6)c6c(c(=O)n7C)OCC(F)(F)C(C7CC7)N6)n5)CC4)CC3)c21] |
| 413a | 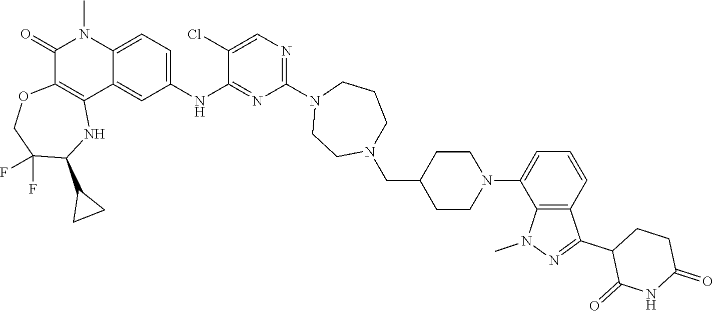 [SMILES: Cn1nc(C2CCC(=O)NC2=O)c2cccc(N3CCC(CN4CCCN(c5ncc(Cl)c(Nc6ccc7c(c6)c6c(c(=O)n7C)OCC(F)(F)[C@H](C7CC7)N6)n5)CC4)CC3)c21] |
| 414 | 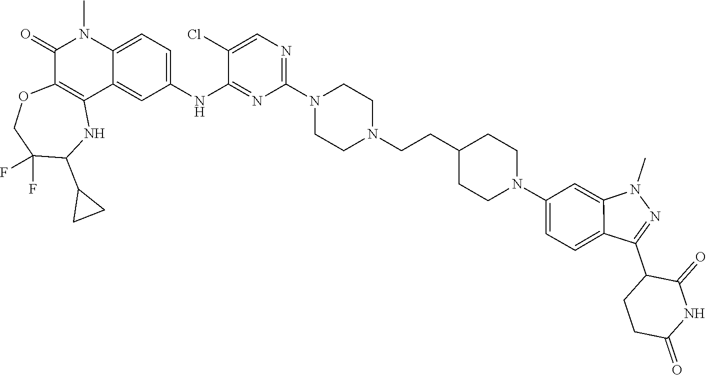 [SMILES: Cn1nc(C2CCC(=O)NC2=O)c2ccc(N3CCC(CCN4CCN(c5ncc(Cl)c(Nc6ccc7c(c6)c6c(c(=O)n7C)OCC(F)(F)C(C7CC7)N6)n5)CC4)CC3)cc21] |
| 414a | 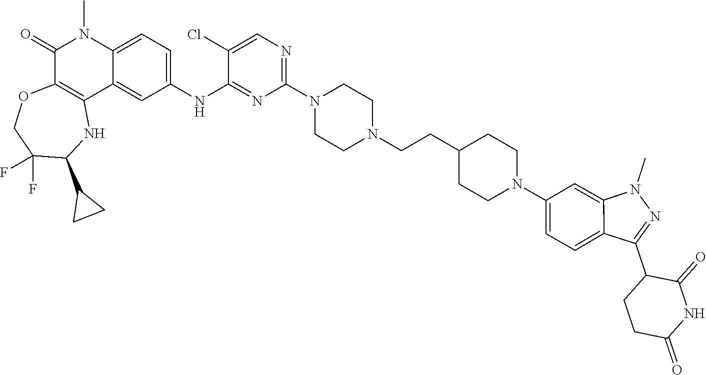 [SMILES: Cn1nc(C2CCC(=O)NC2=O)c2ccc(N3CCC(CCN4CCN(c5ncc(Cl)c(Nc6ccc7c(c6)c6c(c(=O)n7C)OCC(F)(F)[C@H](C7CC7)N6)n5)CC4)CC3)cc21] |
| 415 | 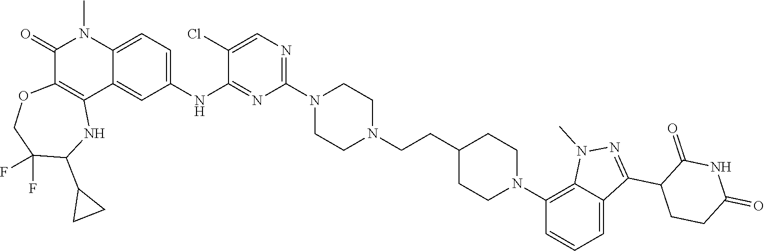 [SMILES: Cn1nc(C2CCC(=O)NC2=O)c2cccc(N3CCC(CCN4CCN(c5ncc(Cl)c(Nc6ccc7c(c6)c6c(c(=O)n7C)OCC(F)(F)C(C7CC7)N6)n5)CC4)CC3)c21] |
| 415a | 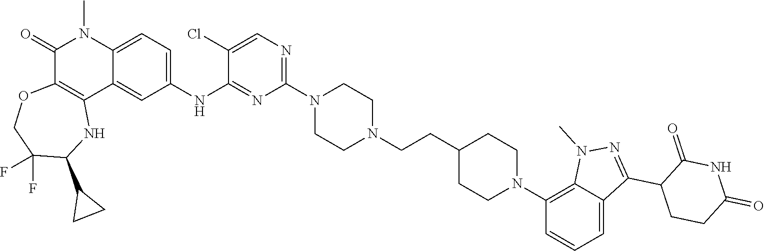 [SMILES: Cn1nc(C2CCC(=O)NC2=O)c2cccc(N3CCC(CCN4CCN(c5ncc(Cl)c(Nc6ccc7c(c6)c6c(c(=O)n7C)OCC(F)(F)[C@H](C7CC7)N6)n5)CC4)CC3)c21] |
| 416 | 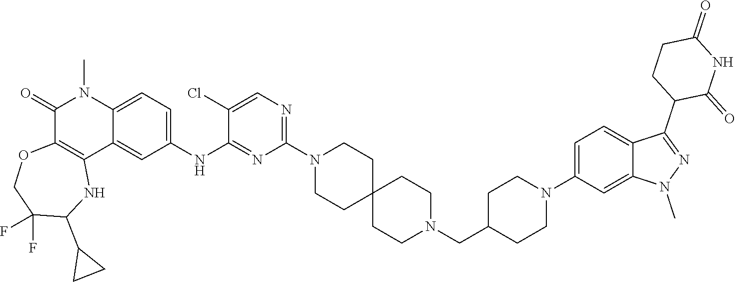 [SMILES: Cn1nc(C2CCC(=O)NC2=O)c2ccc(N3CCC(CN4CCC5(CC4)CCN(c4ncc(Cl)c(Nc6ccc7c(c6)c6c(c(=O)n7C)OCC(F)(F)C(C7CC7)N6)n4)CC5)CC3)cc21] |
| 416a | 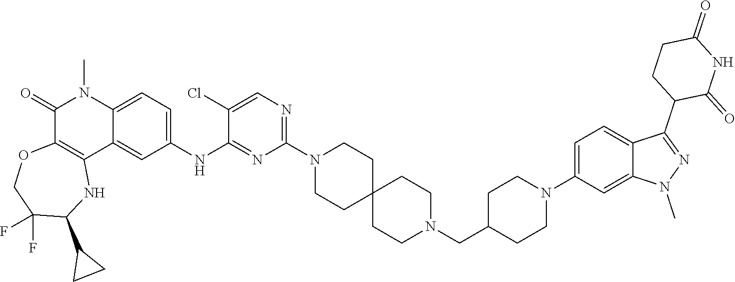 [SMILES: Cn1nc(C2CCC(=O)NC2=O)c2ccc(N3CCC(CN4CCC5(CC4)CCN(c4ncc(Cl)c(Nc6ccc7c(c6)c6c(c(=O)n7C)OCC(F)(F)[C@H](C7CC7)N6)n4)CC5)CC3)cc21] |
| 417 | 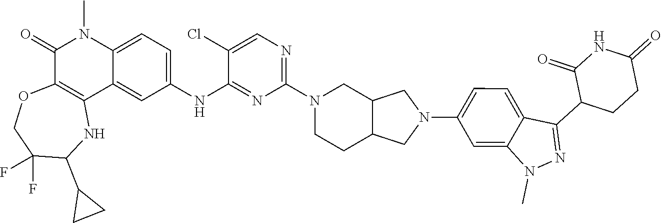 [SMILES: Cn1nc(C2CCC(=O)NC2=O)c2ccc(N3CC4CCN(c5ncc(Cl)c(Nc6ccc7c(c6)c6c(c(=O)n7C)OCC(F)(F)C(C7CC7)N6)n5)CC4C3)cc21] |
| 417a | 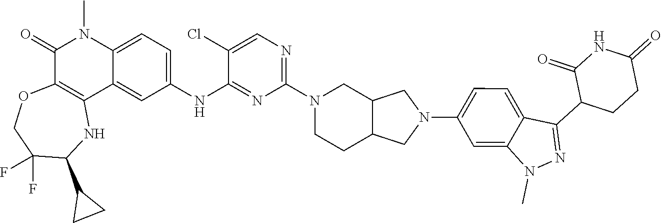 [SMILES: Cn1nc(C2CCC(=O)NC2=O)c2ccc(N3CC4CCN(c5ncc(Cl)c(Nc6ccc7c(c6)c6c(c(=O)n7C)OCC(F)(F)[C@H](C7CC7)N6)n5)CC4C3)cc21] |
| 418 | 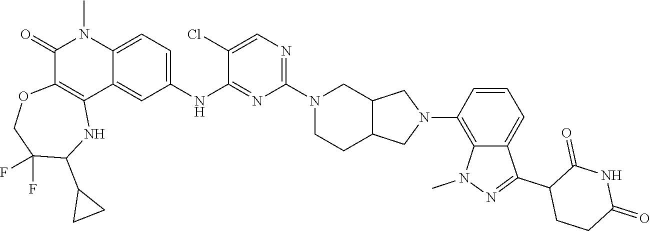 [SMILES: Cn1nc(C2CCC(=O)NC2=O)c2cccc(N3CC4CCN(c5ncc(Cl)c(Nc6ccc7c(c6)c6c(c(=O)n7C)OCC(F)(F)C(C7CC7)N6)n5)CC4C3)c21] |
| 418a | 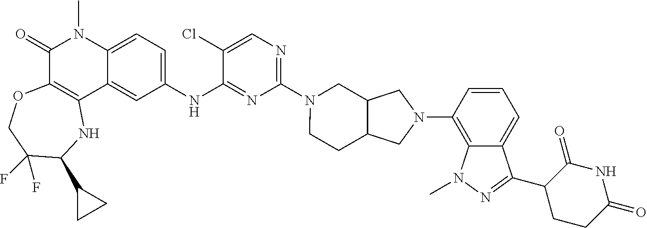 [SMILES: Cn1nc(C2CCC(=O)NC2=O)c2cccc(N3CC4CCN(c5ncc(Cl)c(Nc6ccc7c(c6)c6c(c(=O)n7C)OCC(F)(F)[C@H](C7CC7)N6)n5)CC4C3)c21] |
| 419 | 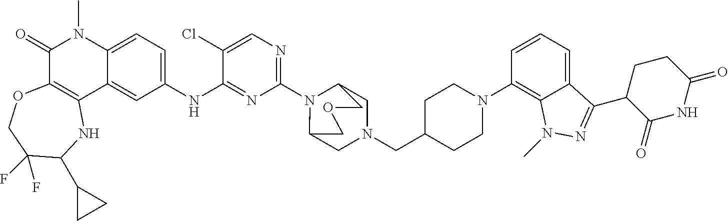 [SMILES: Cn1nc(C2CCC(=O)NC2=O)c2cccc(N3CCC(CN4CC5COCC(C4)N5c4ncc(Cl)c(Nc5ccc6c(c5)c5c(c(=O)n6C)OCC(F)(F)C(C6CC6)N5)n4)CC3)c21] |
| 419a | 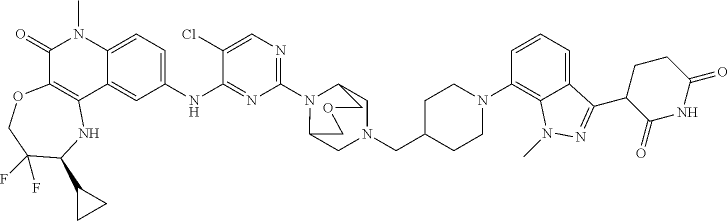 [SMILES: Cn1nc(C2CCC(=O)NC2=O)c2cccc(N3CCC(CN4CC5COCC(C4)N5c4ncc(Cl)c(Nc5ccc6c(c5)c5c(c(=O)n6C)OCC(F)(F)[C@H](C6CC6)N5)n4)CC3)c21] |
| 420 | 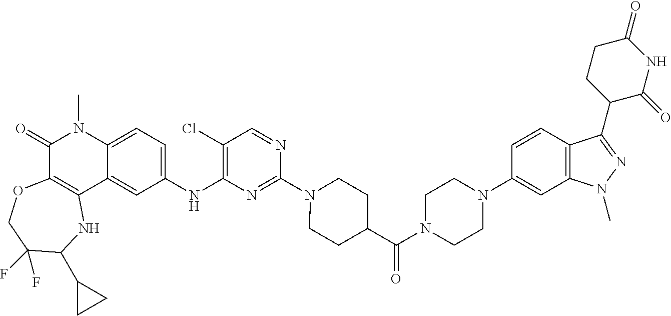 [SMILES: Cn1nc(C2CCC(=O)NC2=O)c2ccc(N3CCN(C(=O)C4CCN(c5ncc(Cl)c(Nc6ccc7c(c6)c6c(c(=O)n7C)OCC(F)(F)C(C7CC7)N6)n5)CC4)CC3)cc21] |
| 420a | 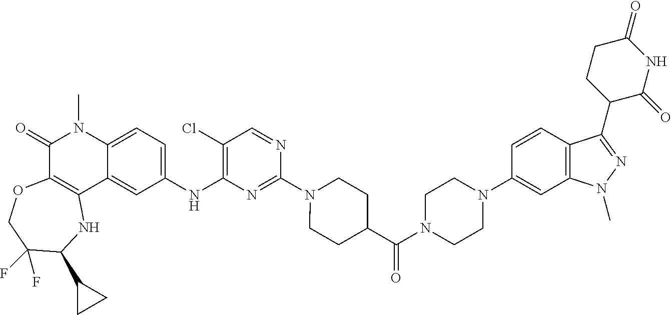 [SMILES: Cn1nc(C2CCC(=O)NC2=O)c2ccc(N3CCN(C(=O)C4CCN(c5ncc(Cl)c(Nc6ccc7c(c6)c6c(c(=O)n7C)OCC(F)(F)[C@H](C7CC7)N6)n5)CC4)CC3)cc21] |
| 421 | 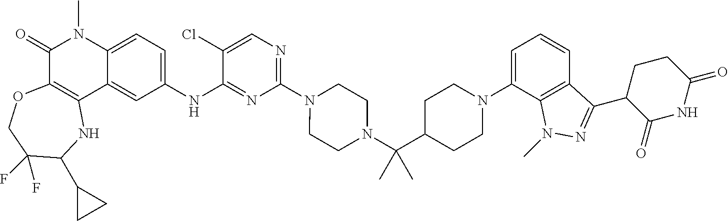 [SMILES: Cn1nc(C2CCC(=O)NC2=O)c2cccc(N3CCC(C(C)(C)N4CCN(c5ncc(Cl)c(Nc6ccc7c(c6)c6c(c(=O)n7C)OCC(F)(F)C(C7CC7)N6)n5)CC4)CC3)c21] |
| 421a | 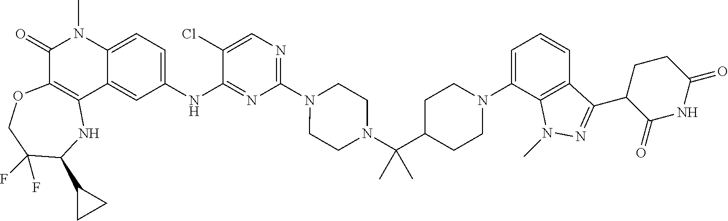 [SMILES: Cn1nc(C2CCC(=O)NC2=O)c2cccc(N3CCC(C(C)(C)N4CCN(c5ncc(Cl)c(Nc6ccc7c(c6)c6c(c(=O)n7C)OCC(F)(F)[C@H](C7CC7)N6)n5)CC4)CC3)c21] |
| 422 | 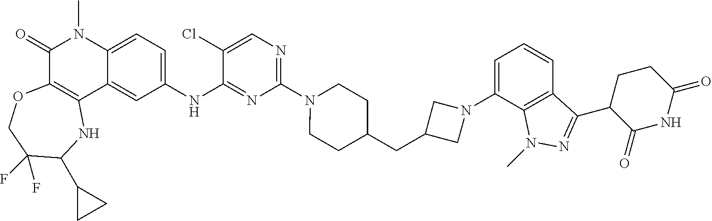 [SMILES: Cn1nc(C2CCC(=O)NC2=O)c2cccc(N3CC(CC4CCN(c5ncc(Cl)c(Nc6ccc7c(c6)c6c(c(=O)n7C)OCC(F)(F)C(C7CC7)N6)n5)CC4)C3)c21] |
| 422a | 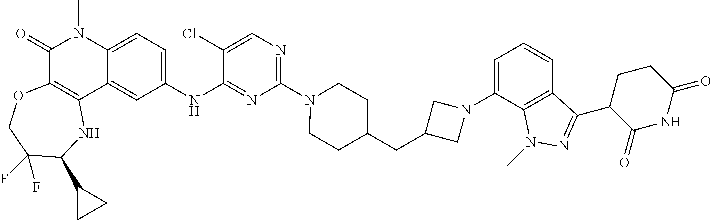 [SMILES: Cn1nc(C2CCC(=O)NC2=O)c2cccc(N3CC(CC4CCN(c5ncc(Cl)c(Nc6ccc7c(c6)c6c(c(=O)n7C)OCC(F)(F)[C@H](C7CC7)N6)n5)CC4)C3)c21] |
| 423 | 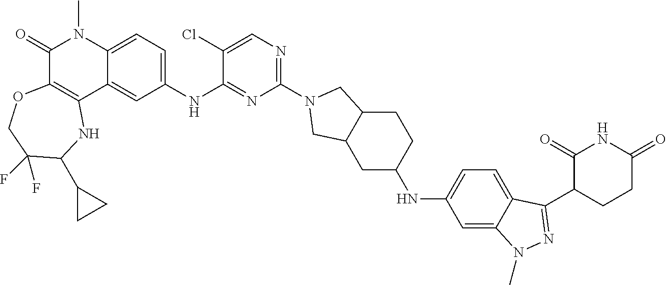 [SMILES: Cn1nc(C2CCC(=O)NC2=O)c2ccc(NC3CCC4CN(c5ncc(Cl)c(Nc6ccc7c(c6)c6c(c(=O)n7C)OCC(F)(F)C(C7CC7)N6)n5)CC4C3)cc21] |
| 423a | 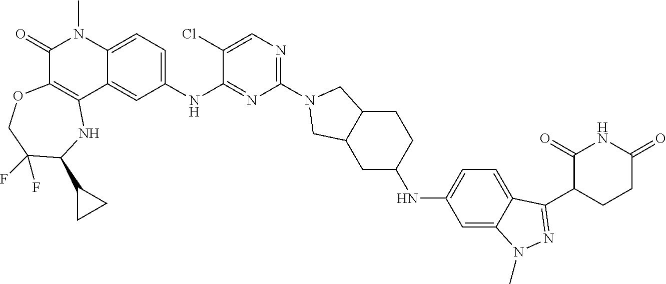 [SMILES: Cn1nc(C2CCC(=O)NC2=O)c2ccc(NC3CCC4CN(c5ncc(Cl)c(Nc6ccc7c(c6)c6c(c(=O)n7C)OCC(F)(F)[C@H](C7CC7)N6)n5)CC4C3)cc21] |
| 424 | 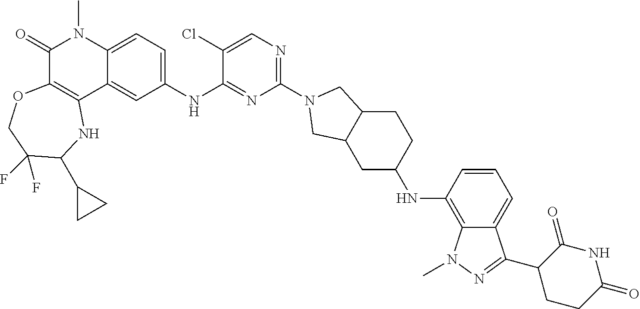 [SMILES: Cn1nc(C2CCC(=O)NC2=O)c2cccc(NC3CCC4CN(c5ncc(Cl)c(Nc6ccc7c(c6)c6c(c(=O)n7C)OCC(F)(F)C(C7CC7)N6)n5)CC4C3)c21] |
| 424a | 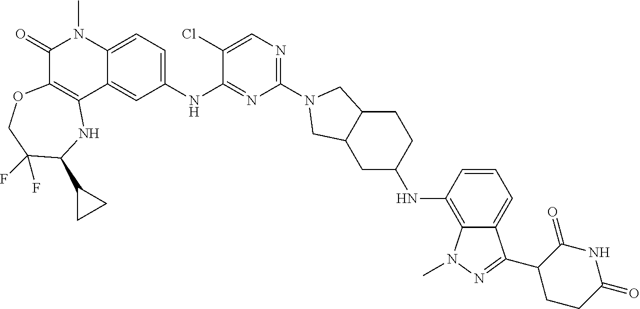 [SMILES: Cn1nc(C2CCC(=O)NC2=O)c2cccc(NC3CCC4CN(c5ncc(Cl)c(Nc6ccc7c(c6)c6c(c(=O)n7C)OCC(F)(F)[C@H](C7CC7)N6)n5)CC4C3)c21] |
| 425 | 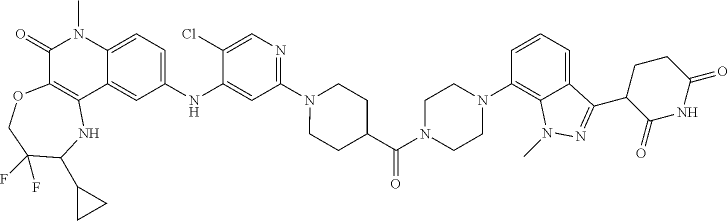 [SMILES: Cn1nc(C2CCC(=O)NC2=O)c2cccc(N3CCN(C(=O)C4CCN(c5cc(Nc6ccc7c(c6)c6c(c(=O)n7C)OCC(F)(F)C(C7CC7)N6)c(Cl)cn5)CC4)CC3)c21] |
| 425a | 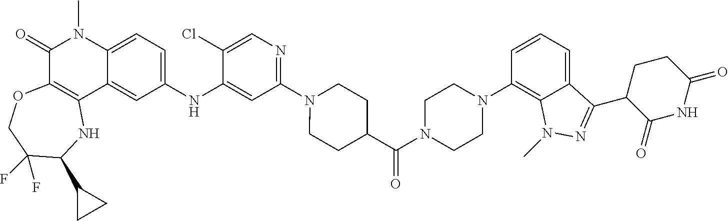 [SMILES: Cn1nc(C2CCC(=O)NC2=O)c2cccc(N3CCN(C(=O)C4CCN(c5cc(Nc6ccc7c(c6)c6c(c(=O)n7C)OCC(F)(F)[C@H](C7CC7)N6)c(Cl)cn5)CC4)CC3)c21] |
| 426 | 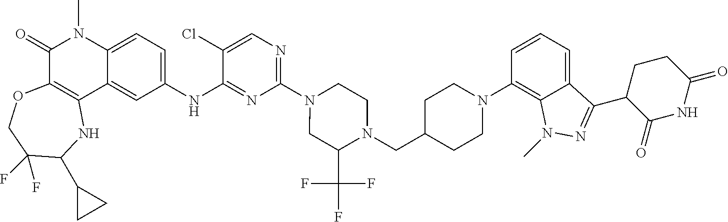 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)CC4C(F)(F)F)CC3)c21] |
| 426a | 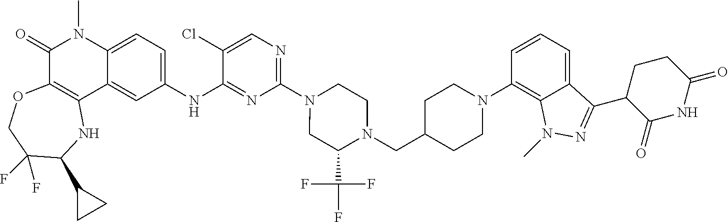 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@H]4C(F)(F)F)CC3)c21] |
| 426b | 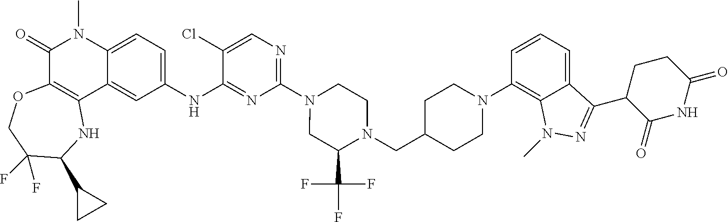 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@@H]4C(F)(F)F)CC3)c21] |
| 427 | 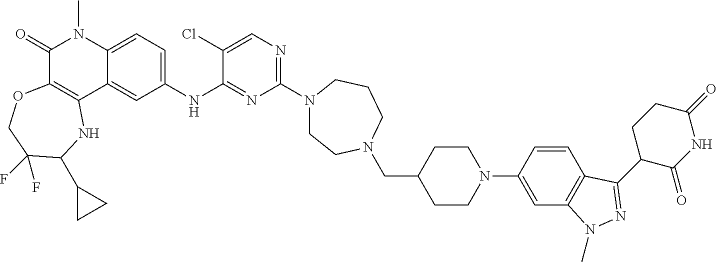 [SMILES: Cn1nc(C2CCC(=O)NC2=O)c2ccc(N3CCC(CN4CCCN(c5ncc(Cl)c(Nc6ccc7c(c6)c6c(c(=O)n7C)OCC(F)(F)C(C7CC7)N6)n5)CC4)CC3)cc21] |
| 427a | 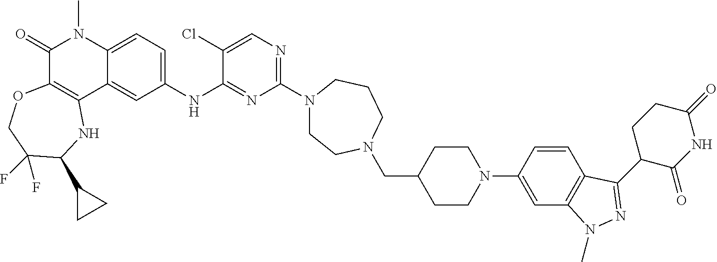 [SMILES: Cn1nc(C2CCC(=O)NC2=O)c2ccc(N3CCC(CN4CCCN(c5ncc(Cl)c(Nc6ccc7c(c6)c6c(c(=O)n7C)OCC(F)(F)[C@H](C7CC7)N6)n5)CC4)CC3)cc21] |
| 428 | 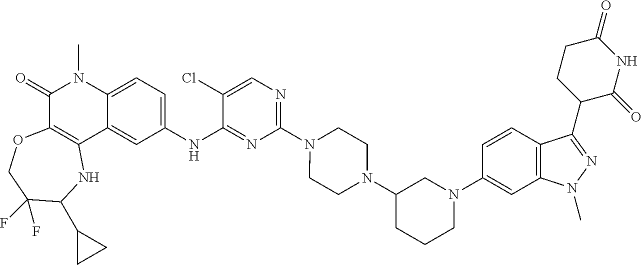 [SMILES: Cn1nc(C2CCC(=O)NC2=O)c2ccc(N3CCCC(N4CCN(c5ncc(Cl)c(Nc6ccc7c(c6)c6c(c(=O)n7C)OCC(F)(F)C(C7CC7)N6)n5)CC4)C3)cc21] |
| 428a | 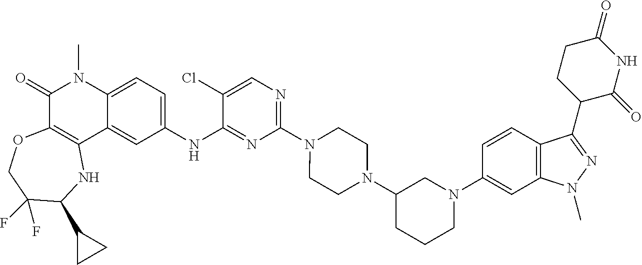 [SMILES: Cn1nc(C2CCC(=O)NC2=O)c2ccc(N3CCCC(N4CCN(c5ncc(Cl)c(Nc6ccc7c(c6)c6c(c(=O)n7C)OCC(F)(F)[C@H](C7CC7)N6)n5)CC4)C3)cc21] |
| 429 | 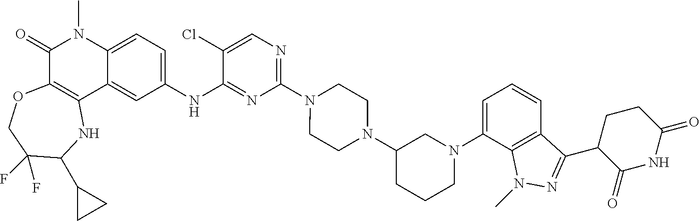 [SMILES: Cn1nc(C2CCC(=O)NC2=O)c2cccc(N3CCCC(N4CCN(c5ncc(Cl)c(Nc6ccc7c(c6)c6c(c(=O)n7C)OCC(F)(F)C(C7CC7)N6)n5)CC4)C3)c21] |
| 429a | 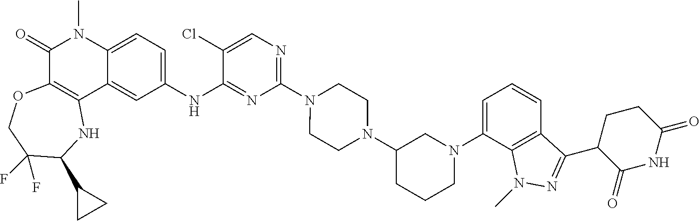 [SMILES: Cn1nc(C2CCC(=O)NC2=O)c2cccc(N3CCCC(N4CCN(c5ncc(Cl)c(Nc6ccc7c(c6)c6c(c(=O)n7C)OCC(F)(F)[C@H](C7CC7)N6)n5)CC4)C3)c21] |
| 430 | 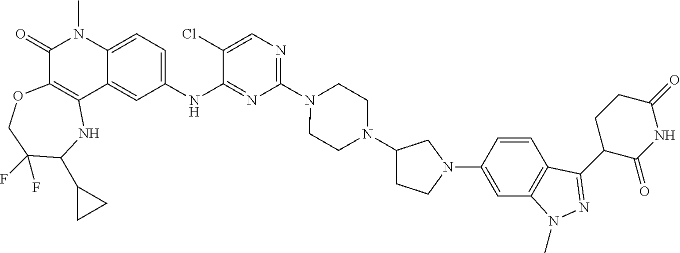 [SMILES: Cn1nc(C2CCC(=O)NC2=O)c2ccc(N3CCC(N4CCN(c5ncc(Cl)c(Nc6ccc7c(c6)c6c(c(=O)n7C)OCC(F)(F)C(C7CC7)N6)n5)CC4)C3)cc21] |
| 430a | 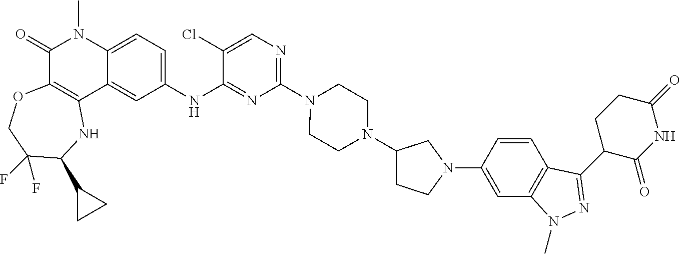 [SMILES: Cn1nc(C2CCC(=O)NC2=O)c2ccc(N3CCC(N4CCN(c5ncc(Cl)c(Nc6ccc7c(c6)c6c(c(=O)n7C)OCC(F)(F)[C@H](C7CC7)N6)n5)CC4)C3)cc21] |
| 431 | 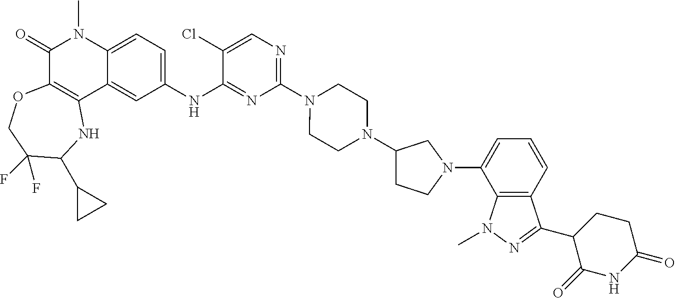 [SMILES: Cn1nc(C2CCC(=O)NC2=O)c2cccc(N3CCC(N4CCN(c5ncc(Cl)c(Nc6ccc7c(c6)c6c(c(=O)n7C)OCC(F)(F)C(C7CC7)N6)n5)CC4)C3)c21] |
| 431a | 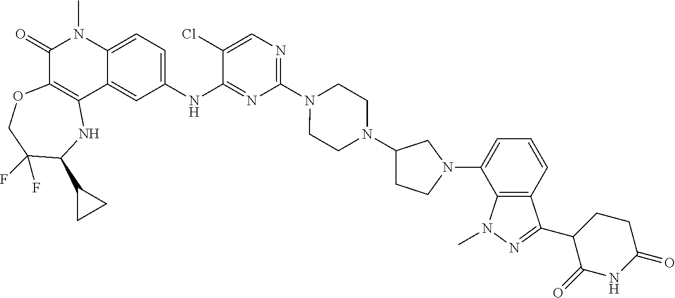 [SMILES: Cn1nc(C2CCC(=O)NC2=O)c2cccc(N3CCC(N4CCN(c5ncc(Cl)c(Nc6ccc7c(c6)c6c(c(=O)n7C)OCC(F)(F)[C@H](C7CC7)N6)n5)CC4)C3)c21] |
| 432 | 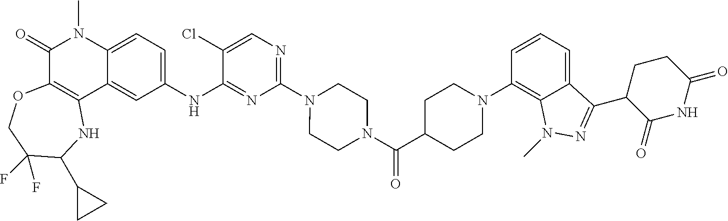 [SMILES: Cn1nc(C2CCC(=O)NC2=O)c2cccc(N3CCC(C(=O)N4CCN(c5ncc(Cl)c(Nc6ccc7c(c6)c6c(c(=O)n7C)OCC(F)(F)C(C7CC7)N6)n5)CC4)CC3)c21] |
| 432a | 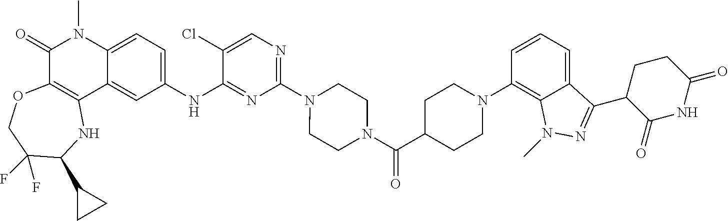 [SMILES: Cn1nc(C2CCC(=O)NC2=O)c2cccc(N3CCC(C(=O)N4CCN(c5ncc(Cl)c(Nc6ccc7c(c6)c6c(c(=O)n7C)OCC(F)(F)[C@H](C7CC7)N6)n5)CC4)CC3)c21] |
| 433 | 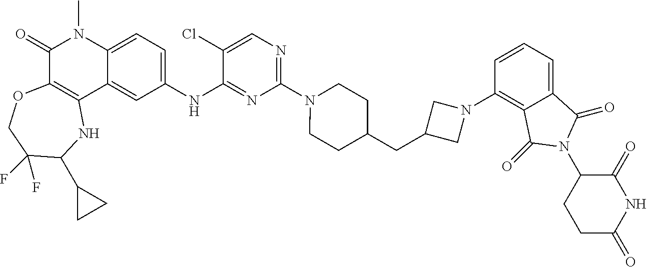 [SMILES: Cn1c(=O)c2c(c3cc(Nc4nc(N5CCC(CC6CN(c7cccc8c7C(=O)N(C7CCC(=O)NC7=O)C8=O)C6)CC5)ncc4Cl)ccc31)NC(C1CC1)C(F)(F)CO2] |
| 433a | 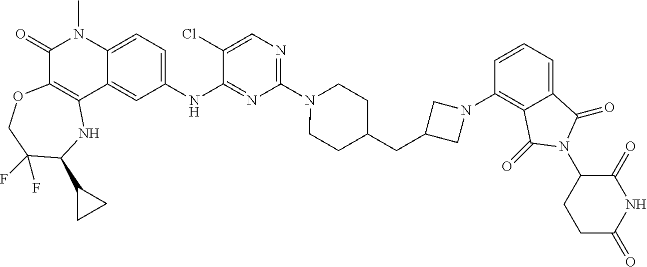 [SMILES: Cn1c(=O)c2c(c3cc(Nc4nc(N5CCC(CC6CN(c7cccc8c7C(=O)N(C7CCC(=O)NC7=O)C8=O)C6)CC5)ncc4Cl)ccc31)N[C@@H](C1CC1)C(F)(F)CO2] |
| 434 | 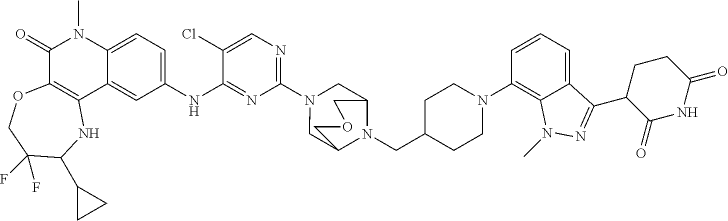 [SMILES: Cn1nc(C2CCC(=O)NC2=O)c2cccc(N3CCC(CN4C5COCC4CN(c4ncc(Cl)c(Nc6ccc7c(c6)c6c(c(=O)n7C)OCC(F)(F)C(C7CC7)N6)n4)C5)CC3)c21] |
| 434a | 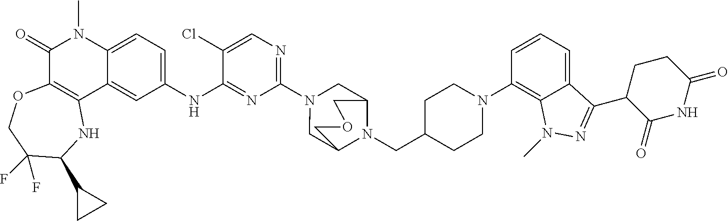 [SMILES: Cn1nc(C2CCC(=O)NC2=O)c2cccc(N3CCC(CN4C5COCC4CN(c4ncc(Cl)c(Nc6ccc7c(c6)c6c(c(=O)n7C)OCC(F)(F)[C@H](C7CC7)N6)n4)C5)CC3)c21] |
| 435 | 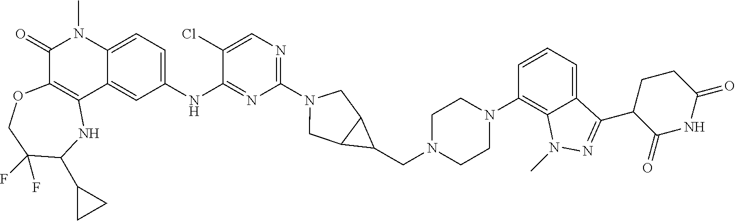 [SMILES: Cn1nc(C2CCC(=O)NC2=O)c2cccc(N3CCN(CC4C5CN(c6ncc(Cl)c(Nc7ccc8c(c7)c7c(c(=O)n8C)OCC(F)(F)C(C8CC8)N7)n6)CC45)CC3)c21] |
| 435a | 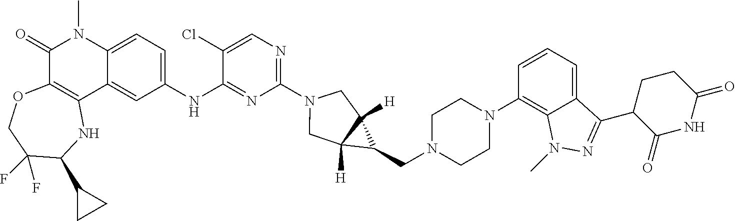 [SMILES: Cn1nc(C2CCC(=O)NC2=O)c2cccc(N3CCN(C[C@@H]4[C@H]5CN(c6ncc(Cl)c(Nc7ccc8c(c7)c7c(c(=O)n8C)OCC(F)(F)[C@H](C8CC8)N7)n6)C[C@@H]45)CC3)c21] |
| 436 | 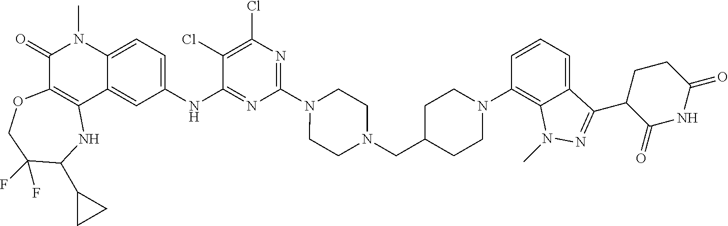 [SMILES: Cn1nc(C2CCC(=O)NC2=O)c2cccc(N3CCC(CN4CCN(c5nc(Cl)c(Cl)c(Nc6ccc7c(c6)c6c(c(=O)n7C)OCC(F)(F)C(C7CC7)N6)n5)CC4)CC3)c21] |
| 436a | 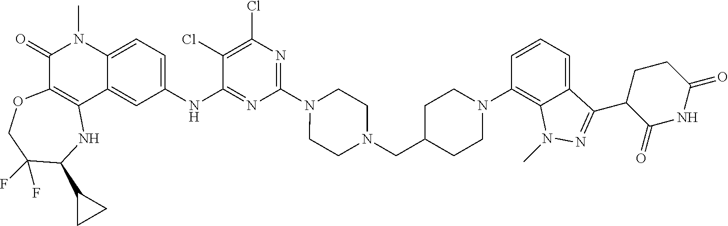 [SMILES: Cn1nc(C2CCC(=O)NC2=O)c2cccc(N3CCC(CN4CCN(c5nc(Cl)c(Cl)c(Nc6ccc7c(c6)c6c(c(=O)n7C)OCC(F)(F)[C@H](C7CC7)N6)n5)CC4)CC3)c21] |
| 437 | 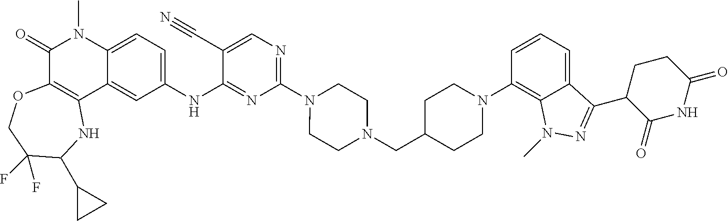 [SMILES: Cn1nc(C2CCC(=O)NC2=O)c2cccc(N3CCC(CN4CCN(c5ncc(C#N)c(Nc6ccc7c(c6)c6c(c(=O)n7C)OCC(F)(F)C(C7CC7)N6)n5)CC4)CC3)c21] |
| 437a | 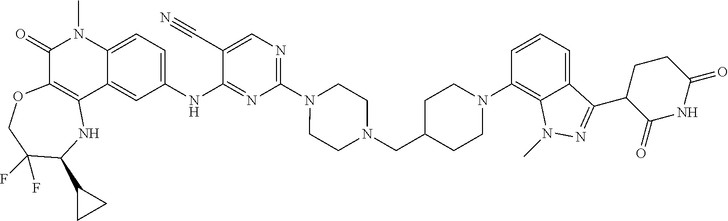 [SMILES: Cn1nc(C2CCC(=O)NC2=O)c2cccc(N3CCC(CN4CCN(c5ncc(C#N)c(Nc6ccc7c(c6)c6c(c(=O)n7C)OCC(F)(F)[C@H](C7CC7)N6)n5)CC4)CC3)c21] |
| 438 | 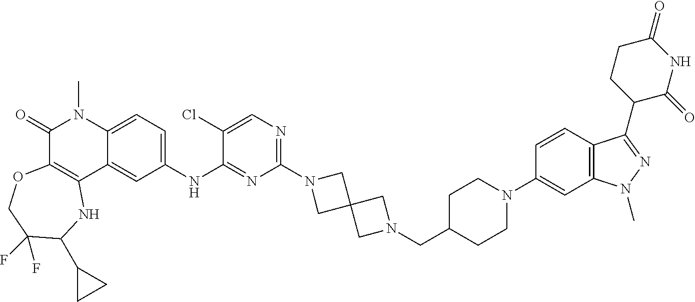 [SMILES: Cn1nc(C2CCC(=O)NC2=O)c2ccc(N3CCC(CN4CC5(C4)CN(c4ncc(Cl)c(Nc6ccc7c(c6)c6c(c(=O)n7C)OCC(F)(F)C(C7CC7)N6)n4)C5)CC3)cc21] |
| 438a | 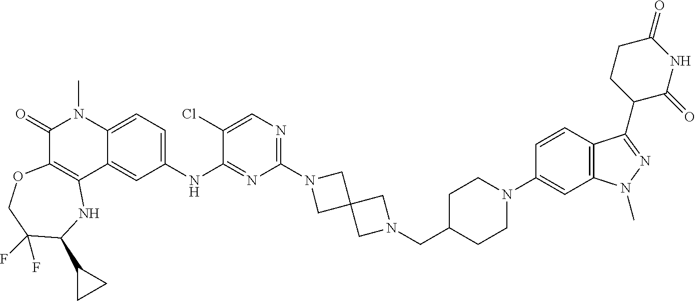 [SMILES: Cn1nc(C2CCC(=O)NC2=O)c2ccc(N3CCC(CN4CC5(C4)CN(c4ncc(Cl)c(Nc6ccc7c(c6)c6c(c(=O)n7C)OCC(F)(F)[C@H](C7CC7)N6)n4)C5)CC3)cc21] |
| 439 | 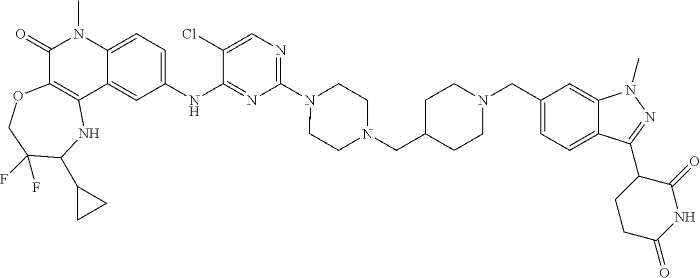 [SMILES: Cn1nc(C2CCC(=O)NC2=O)c2ccc(CN3CCC(CN4CCN(c5ncc(Cl)c(Nc6ccc7c(c6)c6c(c(=O)n7C)OCC(F)(F)C(C7CC7)N6)n5)CC4)CC3)cc21] |
| 439a | 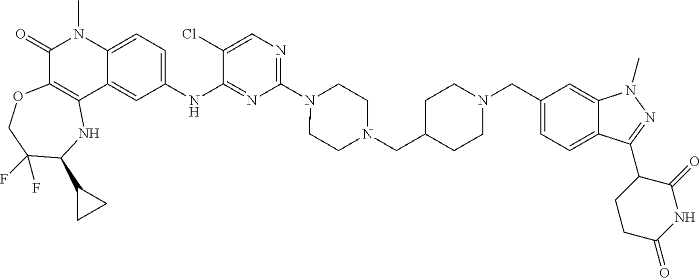 [SMILES: Cn1nc(C2CCC(=O)NC2=O)c2ccc(CN3CCC(CN4CCN(c5ncc(Cl)c(Nc6ccc7c(c6)c6c(c(=O)n7C)OCC(F)(F)[C@H](C7CC7)N6)n5)CC4)CC3)cc21] |
| 440 | 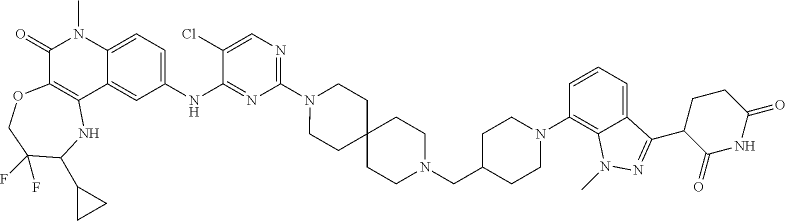 [SMILES: Cn1nc(C2CCC(=O)NC2=O)c2cccc(N3CCC(CN4CCC5(CC4)CCN(c4ncc(Cl)c(Nc6ccc7c(c6)c6c(c(=O)n7C)OCC(F)(F)C(C7CC7)N6)n4)CC5)CC3)c21] |
| 440a | 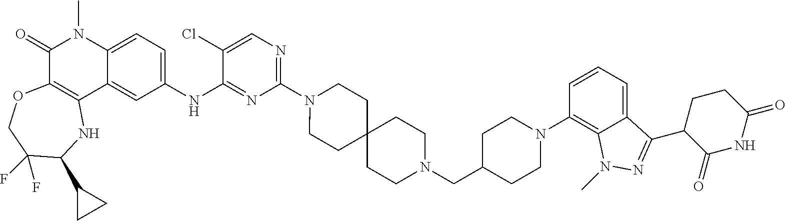 [SMILES: Cn1nc(C2CCC(=O)NC2=O)c2cccc(N3CCC(CN4CCC5(CC4)CCN(c4ncc(Cl)c(Nc6ccc7c(c6)c6c(c(=O)n7C)OCC(F)(F)[C@H](C7CC7)N6)n4)CC5)CC3)c21] |
| 441 | 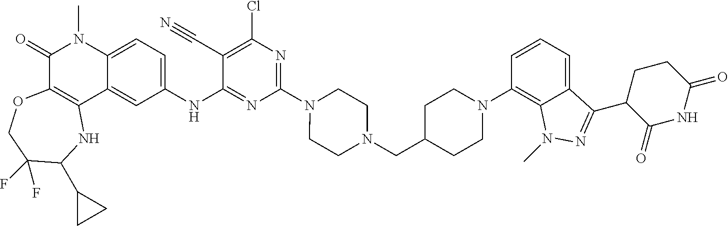 [SMILES: Cn1nc(C2CCC(=O)NC2=O)c2cccc(N3CCC(CN4CCN(c5nc(Cl)c(C#N)c(Nc6ccc7c(c6)c6c(c(=O)n7C)OCC(F)(F)C(C7CC7)N6)n5)CC4)CC3)c21] |
| 441a | 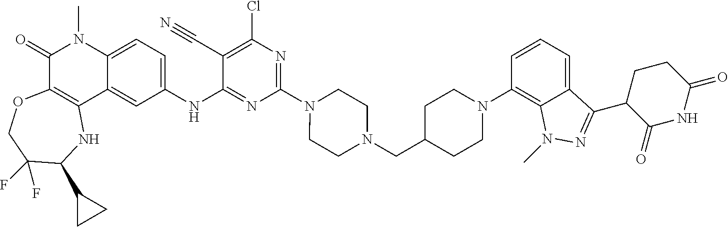 [SMILES: Cn1nc(C2CCC(=O)NC2=O)c2cccc(N3CCC(CN4CCN(c5nc(Cl)c(C#N)c(Nc6ccc7c(c6)c6c(c(=O)n7C)OCC(F)(F)[C@H](C7CC7)N6)n5)CC4)CC3)c21] |
| 442 | 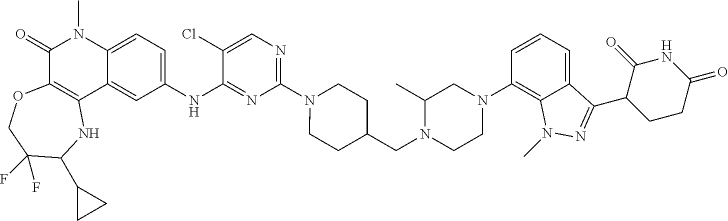 [SMILES: CC1CN(c2cccc3c(C4CCC(=O)NC4=O)nn(C)c23)CCN1CC1CCN(c2ncc(Cl)c(Nc3ccc4c(c3)c3c(c(=O)n4C)OCC(F)(F)C(C4CC4)N3)n2)CC1] |
| 442a | 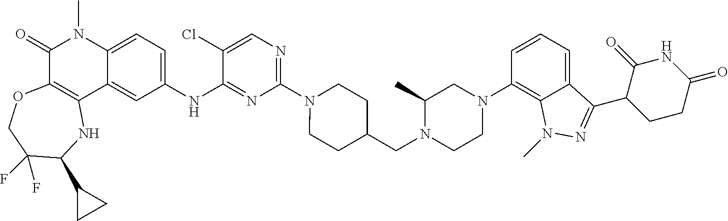 [SMILES: C[C@H]1CN(c2cccc3c(C4CCC(=O)NC4=O)nn(C)c23)CCN1CC1CCN(c2ncc(Cl)c(Nc3ccc4c(c3)c3c(c(=O)n4C)OCC(F)(F)[C@H](C4CC4)N3)n2)CC1] |
| 442b | 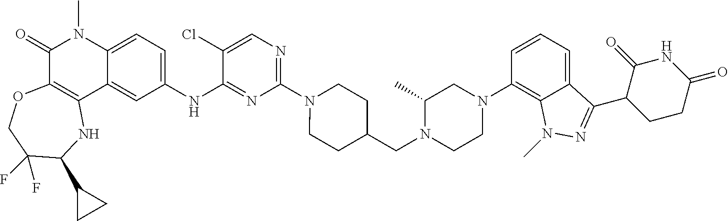 [SMILES: C[C@@H]1CN(c2cccc3c(C4CCC(=O)NC4=O)nn(C)c23)CCN1CC1CCN(c2ncc(Cl)c(Nc3ccc4c(c3)c3c(c(=O)n4C)OCC(F)(F)[C@H](C4CC4)N3)n2)CC1] |
| 443 | 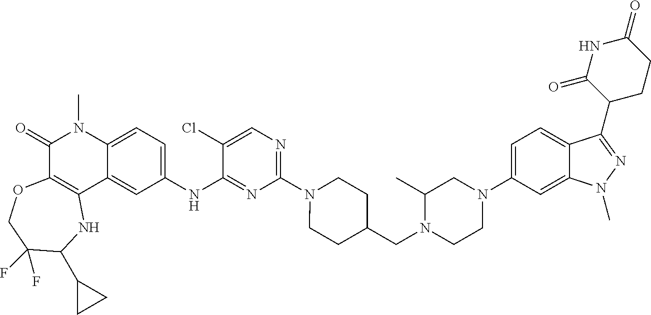 [SMILES: CC1CN(c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)C(C4CC4)N3)n2)CC1] |
| 443a | 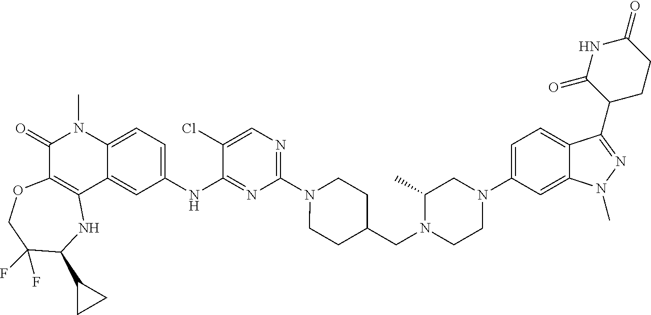 [SMILES: C[C@@H]1CN(c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1] |
| 443b | 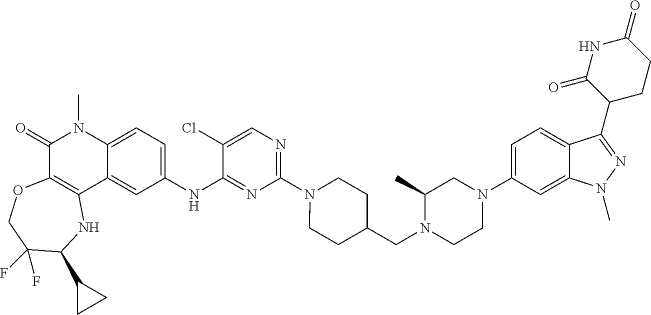 [SMILES: C[C@H]1CN(c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1] |
| 444 | 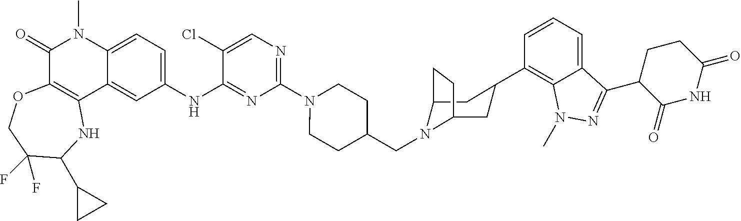 [SMILES: Cn1nc(C2CCC(=O)NC2=O)c2cccc(C3CC4CCC(C3)N4CC3CCN(c4ncc(Cl)c(Nc5ccc6c(c5)c5c(c(=O)n6C)OCC(F)(F)C(C6CC6)N5)n4)CC3)c21] |
| 444a | 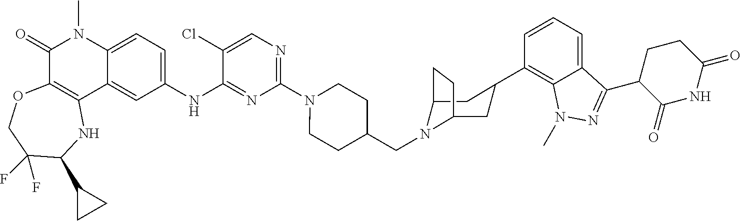 [SMILES: Cn1nc(C2CCC(=O)NC2=O)c2cccc(C3CC4CCC(C3)N4CC3CCN(c4ncc(Cl)c(Nc5ccc6c(c5)c5c(c(=O)n6C)OCC(F)(F)[C@H](C6CC6)N5)n4)CC3)c21] |
| 445 | 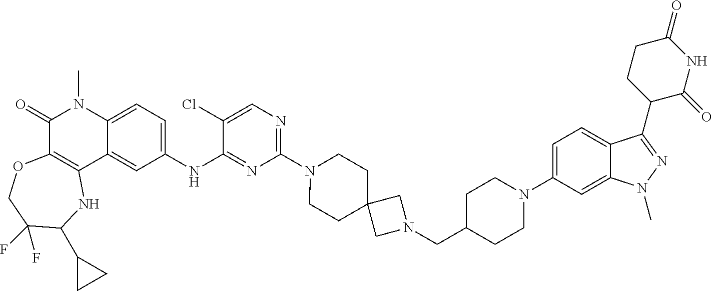 [SMILES: Cn1nc(C2CCC(=O)NC2=O)c2ccc(N3CCC(CN4CC5(CCN(c6ncc(Cl)c(Nc7ccc8c(c7)c7c(c(=O)n8C)OCC(F)(F)C(C8CC8)N7)n6)CC5)C4)CC3)cc21] |
| 445a | 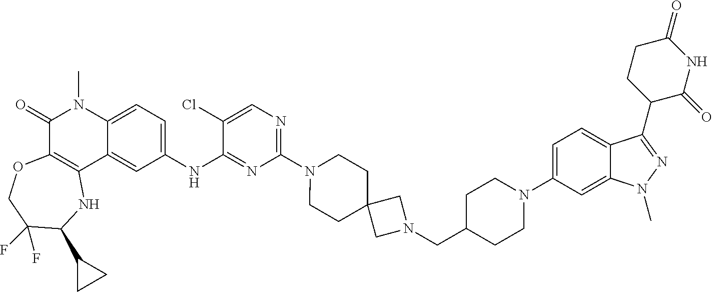 [SMILES: Cn1nc(C2CCC(=O)NC2=O)c2ccc(N3CCC(CN4CC5(CCN(c6ncc(Cl)c(Nc7ccc8c(c7)c7c(c(=O)n8C)OCC(F)(F)[C@H](C8CC8)N7)n6)CC5)C4)CC3)cc21] |
| 446 | 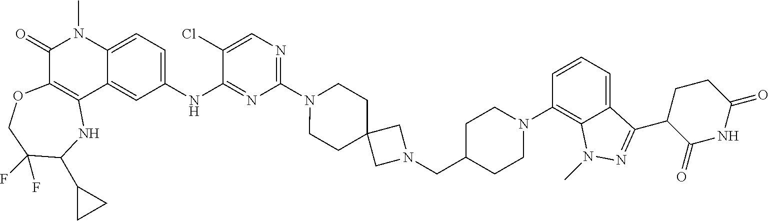 [SMILES: Cn1nc(C2CCC(=O)NC2=O)c2cccc(N3CCC(CN4CC5(CCN(c6ncc(Cl)c(Nc7ccc8c(c7)c7c(c(=O)n8C)OCC(F)(F)C(C8CC8)N7)n6)CC5)C4)CC3)c21] |
| 446a | 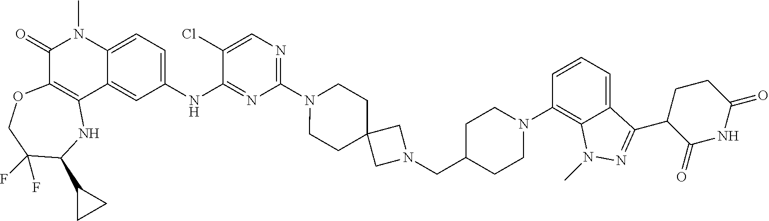 [SMILES: Cn1nc(C2CCC(=O)NC2=O)c2cccc(N3CCC(CN4CC5(CCN(c6ncc(Cl)c(Nc7ccc8c(c7)c7c(c(=O)n8C)OCC(F)(F)[C@H](C8CC8)N7)n6)CC5)C4)CC3)c21] |
| 447 | 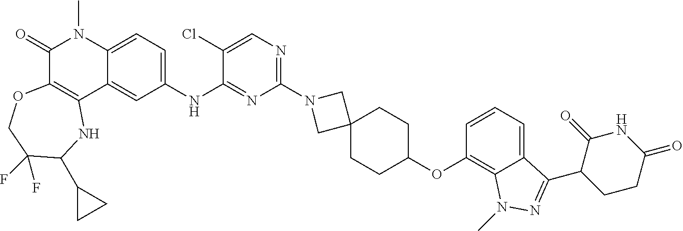 [SMILES: Cn1nc(C2CCC(=O)NC2=O)c2cccc(OC3CCC4(CC3)CN(c3ncc(Cl)c(Nc5ccc6c(c5)c5c(c(=O)n6C)OCC(F)(F)C(C6CC6)N5)n3)C4)c21] |
| 447a | 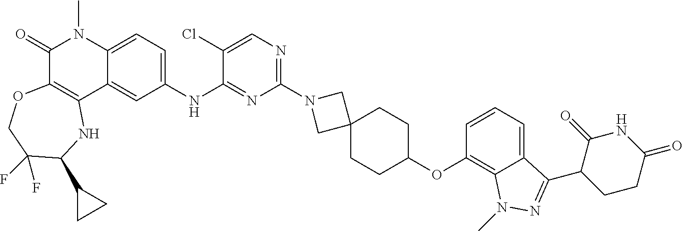 [SMILES: Cn1nc(C2CCC(=O)NC2=O)c2cccc(OC3CCC4(CC3)CN(c3ncc(Cl)c(Nc5ccc6c(c5)c5c(c(=O)n6C)OCC(F)(F)[C@H](C6CC6)N5)n3)C4)c21] |
| 448 | 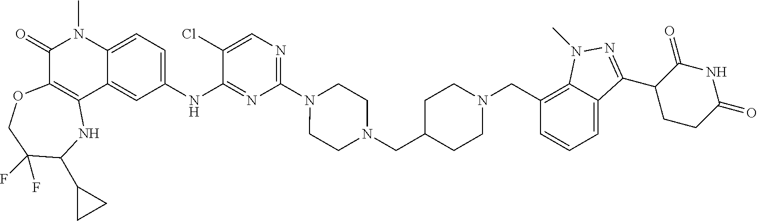 [SMILES: Cn1nc(C2CCC(=O)NC2=O)c2cccc(CN3CCC(CN4CCN(c5ncc(Cl)c(Nc6ccc7c(c6)c6c(c(=O)n7C)OCC(F)(F)C(C7CC7)N6)n5)CC4)CC3)c21] |
| 448a | 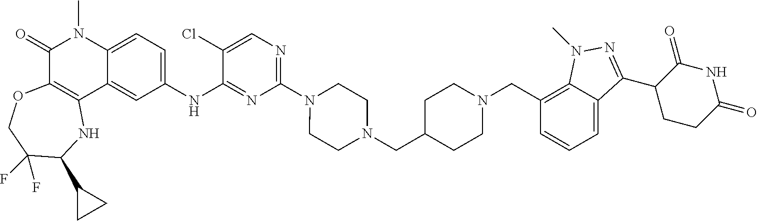 [SMILES: Cn1nc(C2CCC(=O)NC2=O)c2cccc(CN3CCC(CN4CCN(c5ncc(Cl)c(Nc6ccc7c(c6)c6c(c(=O)n7C)OCC(F)(F)[C@H](C7CC7)N6)n5)CC4)CC3)c21] |
| 449 | 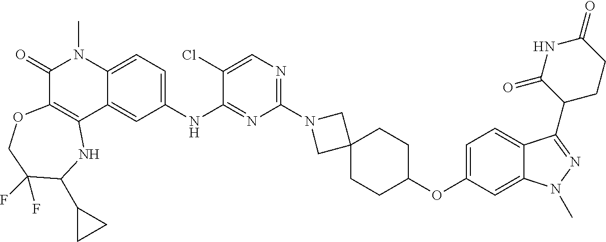 [SMILES: Cn1nc(C2CCC(=O)NC2=O)c2ccc(OC3CCC4(CC3)CN(c3ncc(Cl)c(Nc5ccc6c(c5)c5c(c(=O)n6C)OCC(F)(F)C(C6CC6)N5)n3)C4)cc21] |
| 449a | 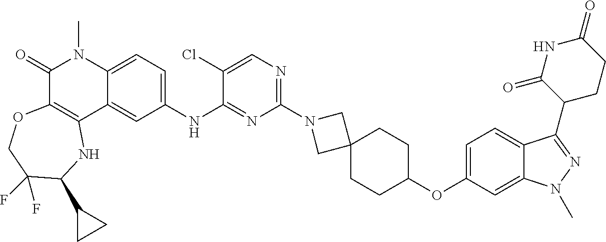 [SMILES: Cn1nc(C2CCC(=O)NC2=O)c2ccc(OC3CCC4(CC3)CN(c3ncc(Cl)c(Nc5ccc6c(c5)c5c(c(=O)n6C)OCC(F)(F)[C@H](C6CC6)N5)n3)C4)cc21] |
| 450 | 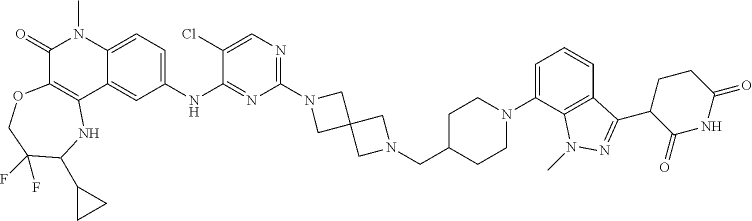 [SMILES: Cn1nc(C2CCC(=O)NC2=O)c2cccc(N3CCC(CN4CC5(C4)CN(c4ncc(Cl)c(Nc6ccc7c(c6)c6c(c(=O)n7C)OCC(F)(F)C(C7CC7)N6)n4)C5)CC3)c21] |
| 450a | 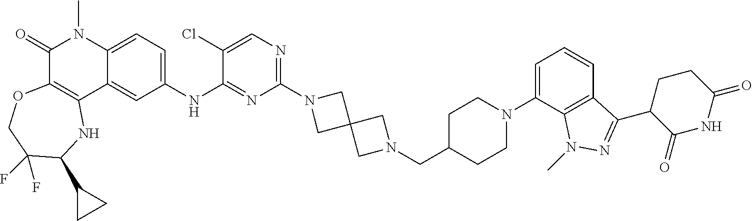 [SMILES: Cn1nc(C2CCC(=O)NC2=O)c2cccc(N3CCC(CN4CC5(C4)CN(c4ncc(Cl)c(Nc6ccc7c(c6)c6c(c(=O)n7C)OCC(F)(F)[C@H](C7CC7)N6)n4)C5)CC3)c21] |
| 451 | 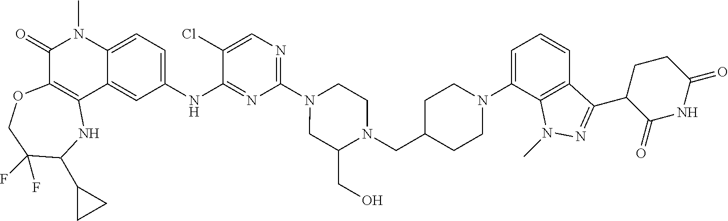 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)CC4CO)CC3)c21] |
| 451a | 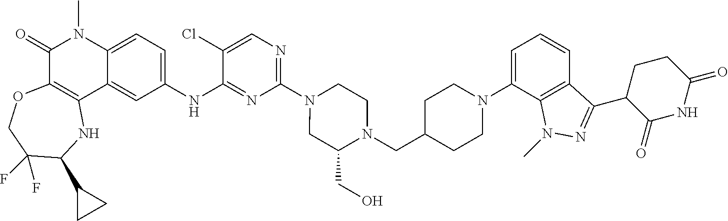 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@H]4CO)CC3)c21] |
| 451b | 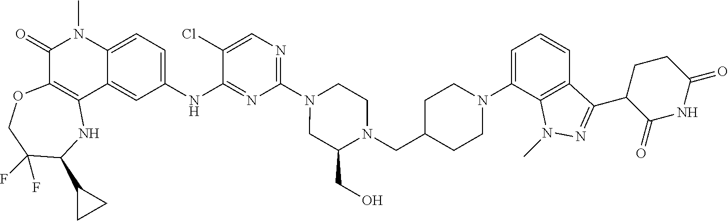 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@@H]4CO)CC3)c21] |
| 452 | 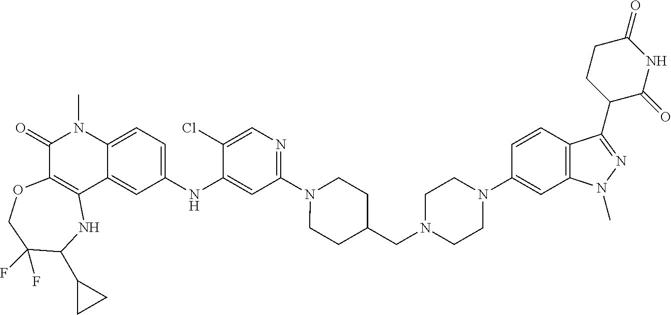 [SMILES: Cn1nc(C2CCC(=O)NC2=O)c2ccc(N3CCN(CC4CCN(c5cc(Nc6ccc7c(c6)c6c(c(=O)n7C)OCC(F)(F)C(C7CC7)N6)c(Cl)cn5)CC4)CC3)cc21] |
| 452a | 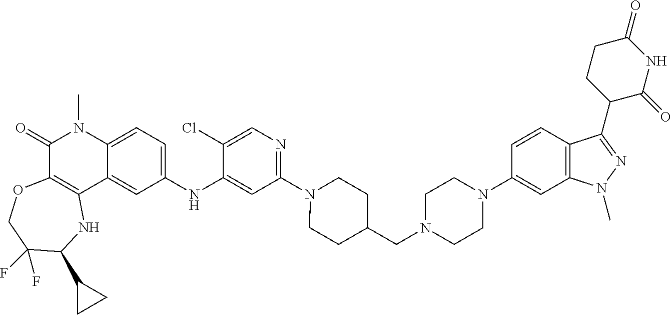 [SMILES: Cn1nc(C2CCC(=O)NC2=O)c2ccc(N3CCN(CC4CCN(c5cc(Nc6ccc7c(c6)c6c(c(=O)n7C)OCC(F)(F)[C@H](C7CC7)N6)c(Cl)cn5)CC4)CC3)cc21] |
| 453 | 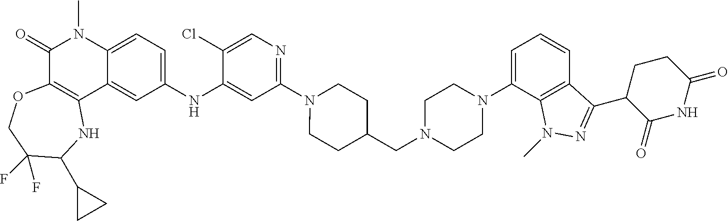 [SMILES: Cn1nc(C2CCC(=O)NC2=O)c2cccc(N3CCN(CC4CCN(c5cc(Nc6ccc7c(c6)c6c(c(=O)n7C)OCC(F)(F)C(C7CC7)N6)c(Cl)cn5)CC4)CC3)c21] |
| 453a | 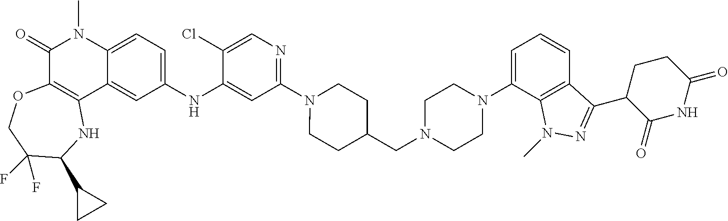 [SMILES: Cn1nc(C2CCC(=O)NC2=O)c2cccc(N3CCN(CC4CCN(c5cc(Nc6ccc7c(c6)c6c(c(=O)n7C)OCC(F)(F)[C@H](C7CC7)N6)c(Cl)cn5)CC4)CC3)c21] |
| 454 | 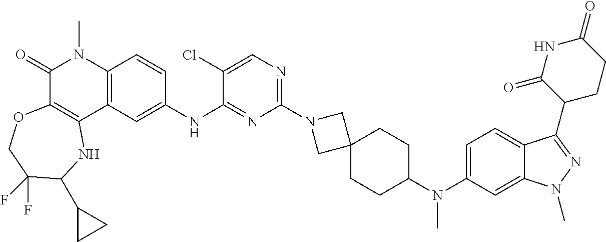 [SMILES: CN(c1ccc2c(C3CCC(=O)NC3=O)nn(C)c2c1)C1CCC2(CC1)CN(c1ncc(Cl)c(Nc3ccc4c(c3)c3c(c(=O)n4C)OCC(F)(F)C(C4CC4)N3)n1)C2] |
| 454a | 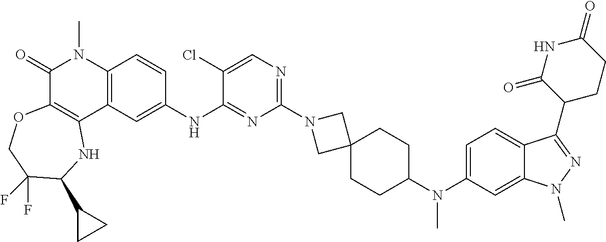 [SMILES: CN(c1ccc2c(C3CCC(=O)NC3=O)nn(C)c2c1)C1CCC2(CC1)CN(c1ncc(Cl)c(Nc3ccc4c(c3)c3c(c(=O)n4C)OCC(F)(F)[C@H](C4CC4)N3)n1)C2] |
| 455 | 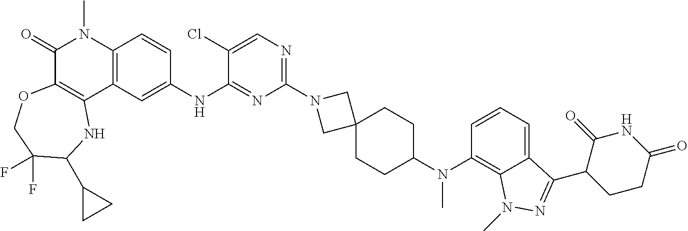 [SMILES: CN(c1cccc2c(C3CCC(=O)NC3=O)nn(C)c12)C1CCC2(CC1)CN(c1ncc(Cl)c(Nc3ccc4c(c3)c3c(c(=O)n4C)OCC(F)(F)C(C4CC4)N3)n1)C2] |
| 455a | 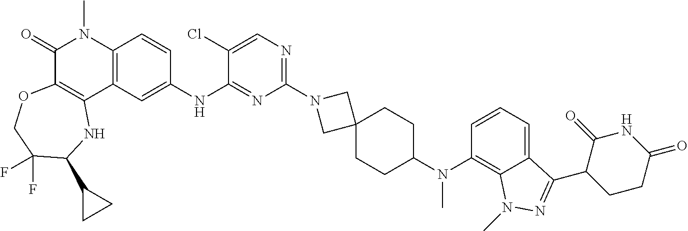 [SMILES: CN(c1cccc2c(C3CCC(=O)NC3=O)nn(C)c12)C1CCC2(CC1)CN(c1ncc(Cl)c(Nc3ccc4c(c3)c3c(c(=O)n4C)OCC(F)(F)[C@H](C4CC4)N3)n1)C2] |
| 456 | 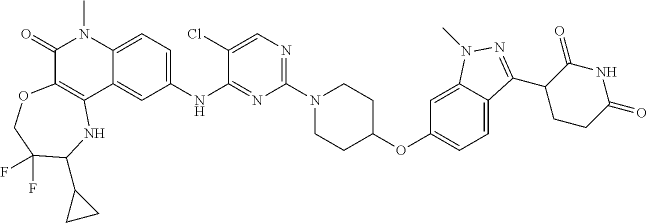 [SMILES: Cn1nc(C2CCC(=O)NC2=O)c2ccc(OC3CCN(c4ncc(Cl)c(Nc5ccc6c(c5)c5c(c(=O)n6C)OCC(F)(F)C(C6CC6)N5)n4)CC3)cc21] |
| 456a | 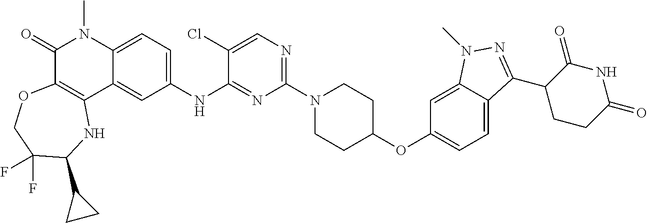 [SMILES: Cn1nc(C2CCC(=O)NC2=O)c2ccc(OC3CCN(c4ncc(Cl)c(Nc5ccc6c(c5)c5c(c(=O)n6C)OCC(F)(F)[C@H](C6CC6)N5)n4)CC3)cc21] |
| 457 | 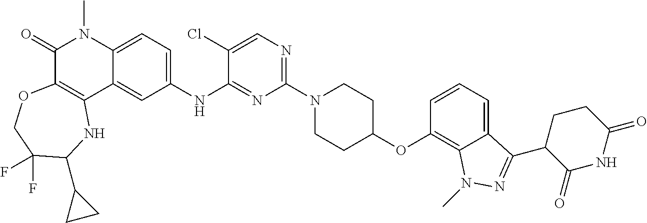 [SMILES: Cn1nc(C2CCC(=O)NC2=O)c2cccc(OC3CCN(c4ncc(Cl)c(Nc5ccc6c(c5)c5c(c(=O)n6C)OCC(F)(F)C(C6CC6)N5)n4)CC3)c21] |
| 457a | 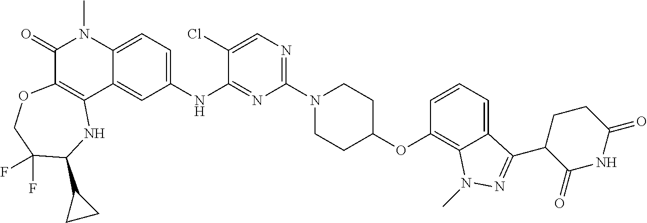 [SMILES: Cn1nc(C2CCC(=O)NC2=O)c2cccc(OC3CCN(c4ncc(Cl)c(Nc5ccc6c(c5)c5c(c(=O)n6C)OCC(F)(F)[C@H](C6CC6)N5)n4)CC3)c21] |
| 458 | 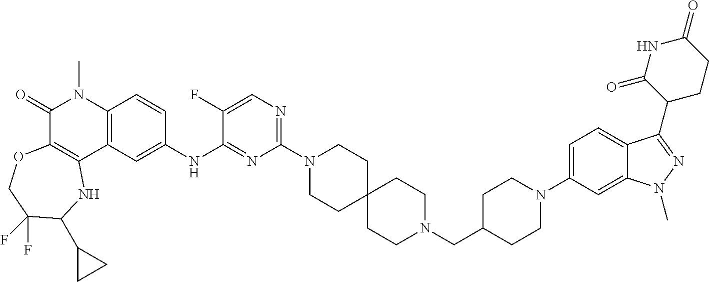 [SMILES: Cn1nc(C2CCC(=O)NC2=O)c2ccc(N3CCC(CN4CCC5(CC4)CCN(c4ncc(F)c(Nc6ccc7c(c6)c6c(c(=O)n7C)OCC(F)(F)C(C7CC7)N6)n4)CC5)CC3)cc21] |
| 458a | 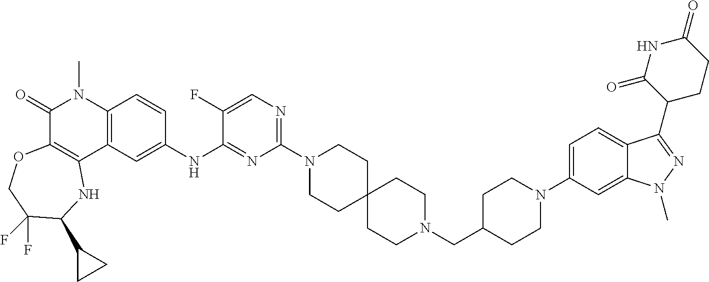 [SMILES: Cn1nc(C2CCC(=O)NC2=O)c2ccc(N3CCC(CN4CCC5(CC4)CCN(c4ncc(F)c(Nc6ccc7c(c6)c6c(c(=O)n7C)OCC(F)(F)[C@H](C7CC7)N6)n4)CC5)CC3)cc21] |
| 459 | 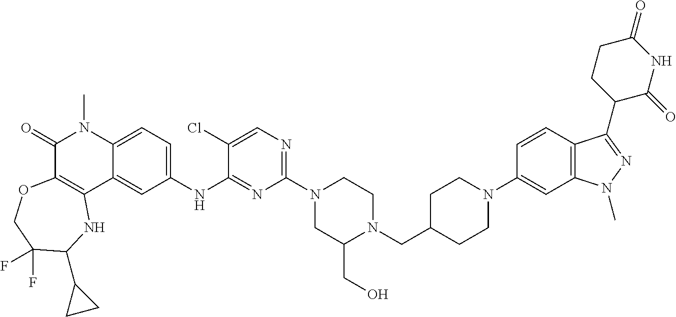 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)C(C7CC7)N6)n5)CC4CO)CC3)cc21] |
| 459a | 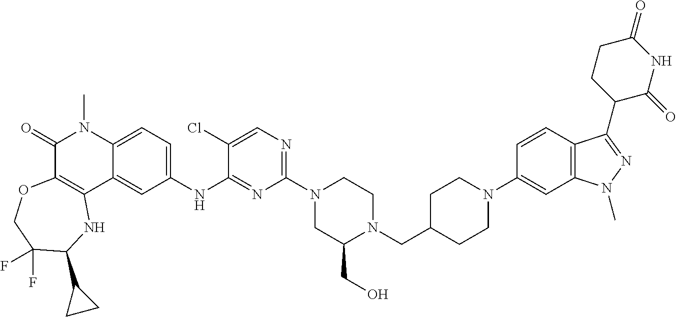 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)C[C@@H]4CO)CC3)cc21] |
| 459b | 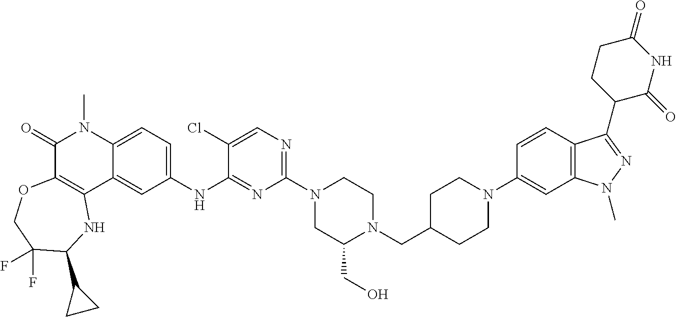 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)C[C@H]4CO)CC3)cc21] |
| 460 | 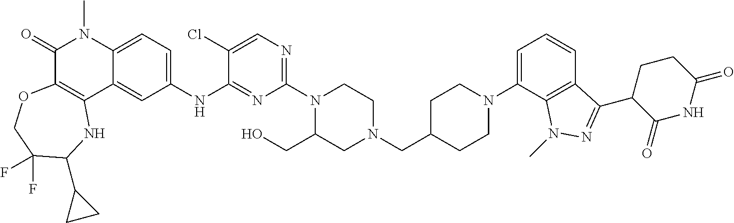 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)C(CO)C4)CC3)c21] |
| 460a | 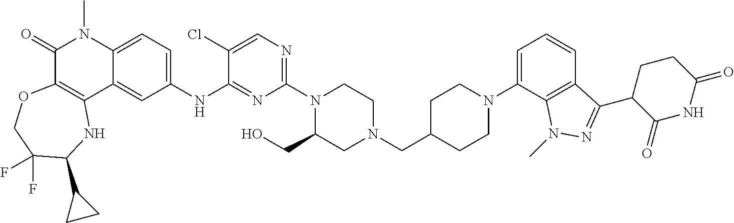 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)[C@H](CO)C4)CC3)c21] |
| 460b | 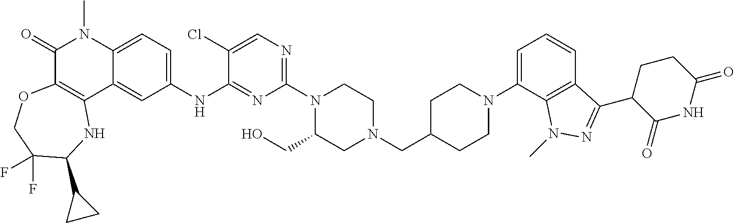 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)[C@@H](CO)C4)CC3)c21] |
| 461 | 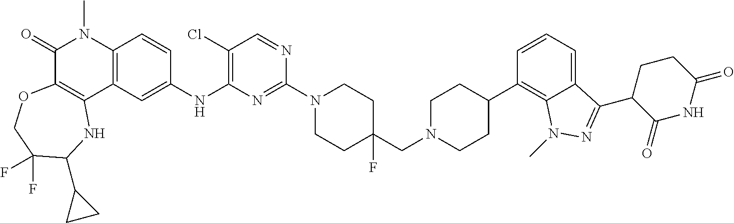 [SMILES: Cn1nc(C2CCC(=O)NC2=O)c2cccc(C3CCN(CC4(F)CCN(c5ncc(Cl)c(Nc6ccc7c(c6)c6c(c(=O)n7C)OCC(F)(F)C(C7CC7)N6)n5)CC4)CC3)c21] |
| 461a | 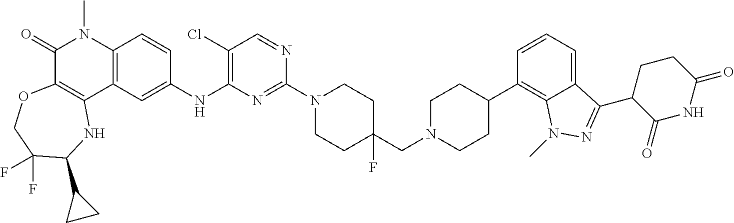 [SMILES: Cn1nc(C2CCC(=O)NC2=O)c2cccc(C3CCN(CC4(F)CCN(c5ncc(Cl)c(Nc6ccc7c(c6)c6c(c(=O)n7C)OCC(F)(F)[C@H](C7CC7)N6)n5)CC4)CC3)c21] |
| 462 | 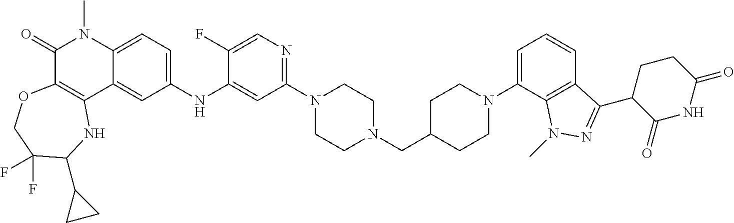 [SMILES: Cn1nc(C2CCC(=O)NC2=O)c2cccc(N3CCC(CN4CCN(c5cc(Nc6ccc7c(c6)c6c(c(=O)n7C)OCC(F)(F)C(C7CC7)N6)c(F)cn5)CC4)CC3)c21] |
| 462a | 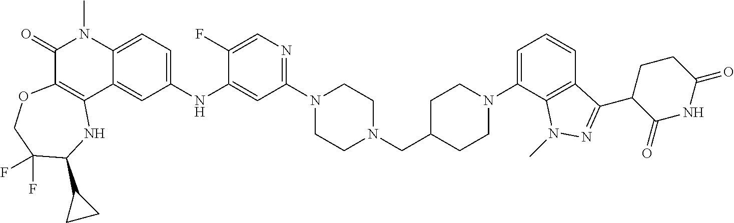 [SMILES: Cn1nc(C2CCC(=O)NC2=O)c2cccc(N3CCC(CN4CCN(c5cc(Nc6ccc7c(c6)c6c(c(=O)n7C)OCC(F)(F)[C@H](C7CC7)N6)c(F)cn5)CC4)CC3)c21] |
| 463 | 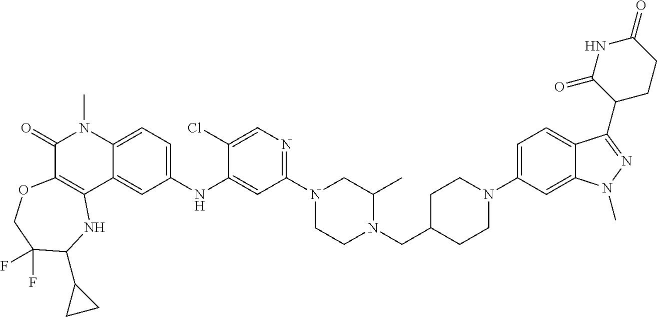 [SMILES: CC1CN(c2cc(Nc3ccc4c(c3)c3c(c(=O)n4C)OCC(F)(F)C(C4CC4)N3)c(Cl)cn2)CCN1CC1CCN(c2ccc3c(C4CCC(=O)NC4=O)nn(C)c3c2)CC1] |
| 463a | 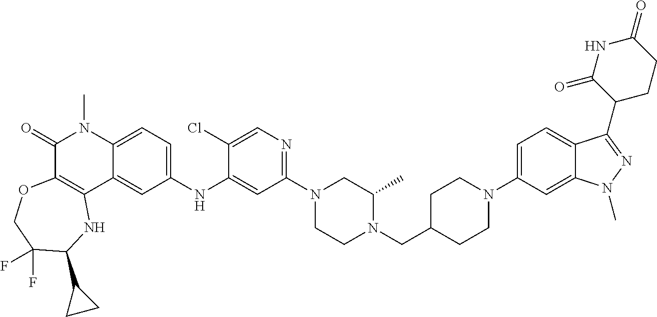 [SMILES: C[C@H]1CN(c2cc(Nc3ccc4c(c3)c3c(c(=O)n4C)OCC(F)(F)[C@H](C4CC4)N3)c(Cl)cn2)CCN1CC1CCN(c2ccc3c(C4CCC(=O)NC4=O)nn(C)c3c2)CC1] |
| 464 | 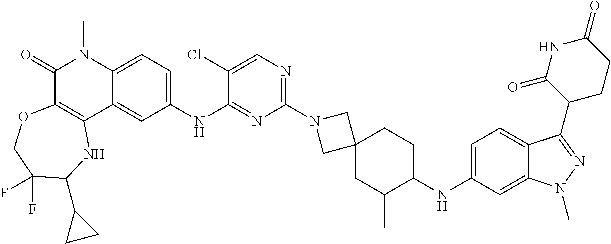 [SMILES: CC1CC2(CCC1Nc1ccc3c(C4CCC(=O)NC4=O)nn(C)c3c1)CN(c1ncc(Cl)c(Nc3ccc4c(c3)c3c(c(=O)n4C)OCC(F)(F)C(C4CC4)N3)n1)C2] |
| 464a | 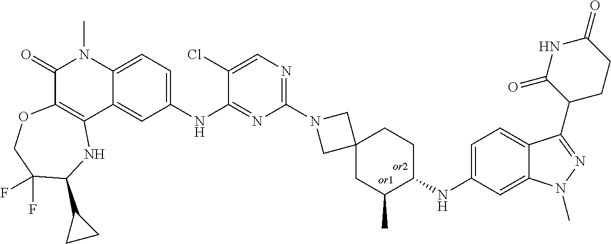 [SMILES: C[C@H]1CC2(CC[C@@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 464b | 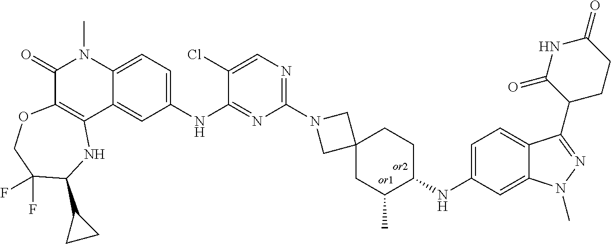 [SMILES: C[C@@H]1CC2(CC[C@@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 464c | 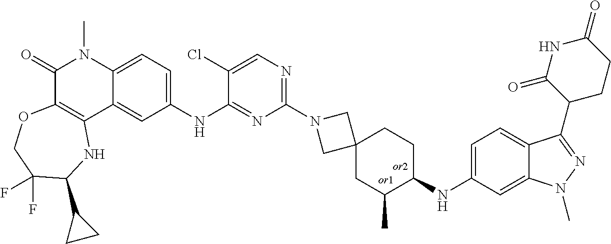 [SMILES: C[C@H]1CC2(CC[C@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 464d | 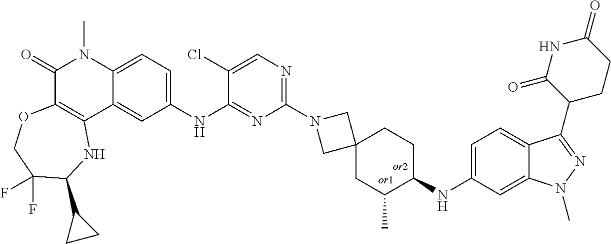 [SMILES: C[C@@H]1CC2(CC[C@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 465 | 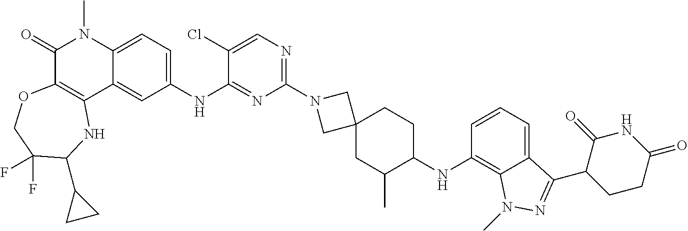 [SMILES: CC1CC2(CCC1Nc1cccc3c(C4CCC(=O)NC4=O)nn(C)c13)CN(c1ncc(Cl)c(Nc3ccc4c(c3)c3c(c(=O)n4C)OCC(F)(F)C(C4CC4)N3)n1)C2] |
| 465a | 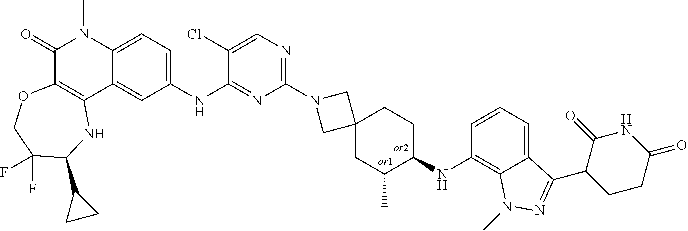 [SMILES: C[C@@H]1CC2(CC[C@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| 465b | 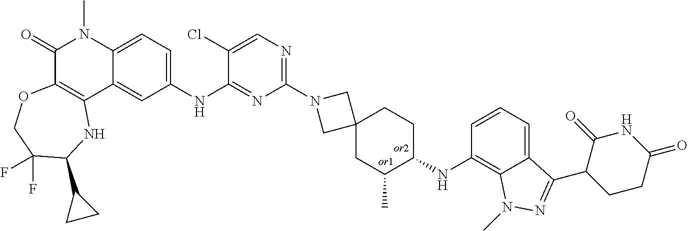 [SMILES: C[C@@H]1CC2(CC[C@@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| 465c | 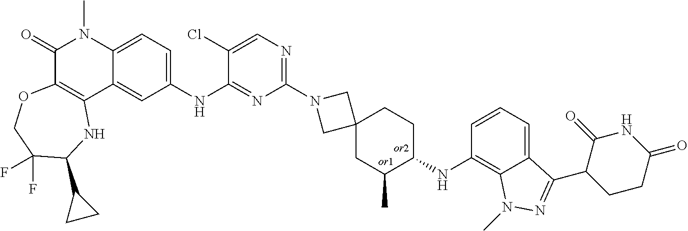 [SMILES: C[C@H]1CC2(CC[C@@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| 465d | 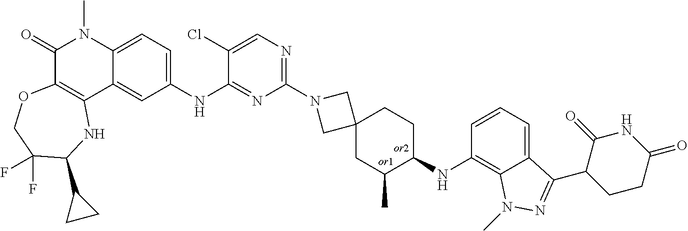 [SMILES: C[C@H]1CC2(CC[C@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| 466 | 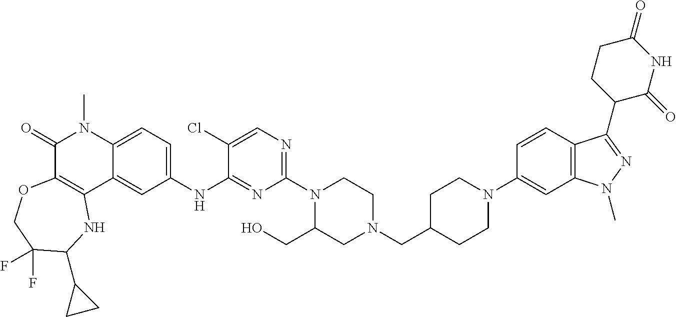 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)C(C7CC7)N6)n5)C(CO)C4)CC3)cc21] |
| 466a | 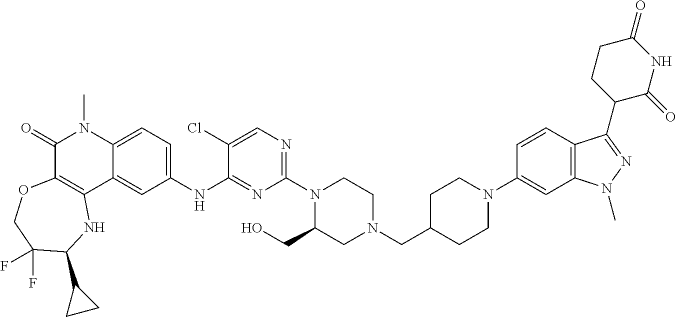 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)[C@H](CO)C4)CC3)cc21] |
| 466b | 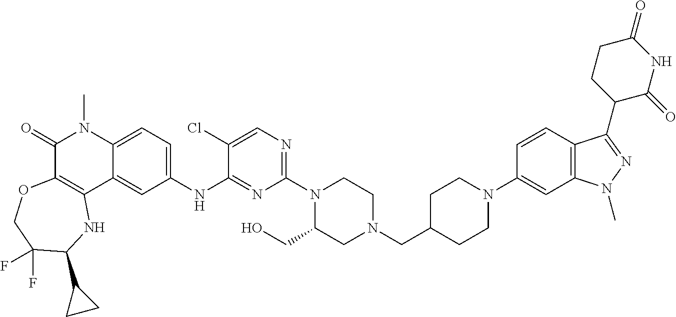 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)[C@@H](CO)C4)CC3)cc21] |
| 467 | 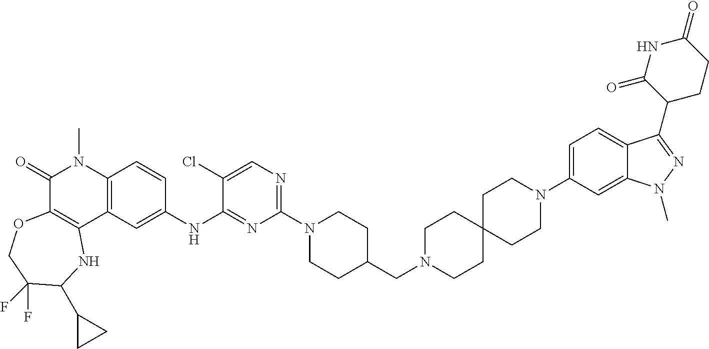 [SMILES: Cn1nc(C2CCC(=O)NC2=O)c2ccc(N3CCC4(CCN(CC5CCN(c6ncc(Cl)c(Nc7ccc8c(c7)c7c(c(=O)n8C)OCC(F)(F)C(C8CC8)N7)n6)CC5)CC4)CC3)cc21] |
| 467a | 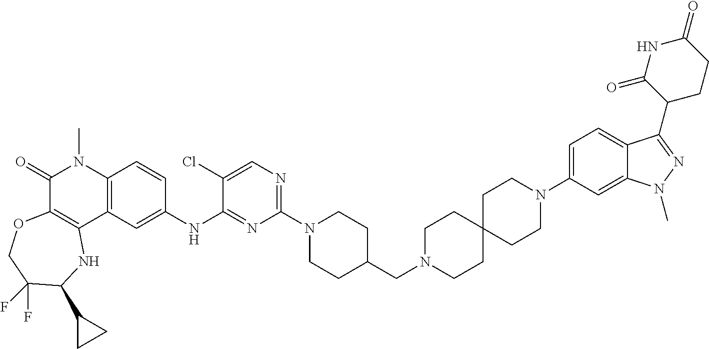 [SMILES: Cn1nc(C2CCC(=O)NC2=O)c2ccc(N3CCC4(CCN(CC5CCN(c6ncc(Cl)c(Nc7ccc8c(c7)c7c(c(=O)n8C)OCC(F)(F)[C@H](C8CC8)N7)n6)CC5)CC4)CC3)cc21] |
| 468 | 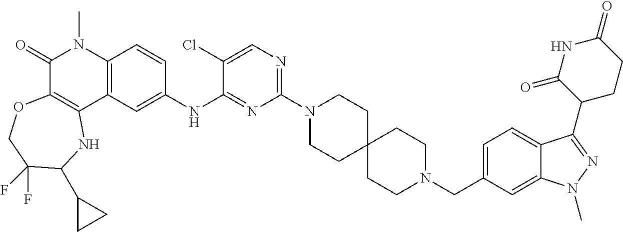 [SMILES: Cn1nc(C2CCC(=O)NC2=O)c2ccc(CN3CCC4(CC3)CCN(c3ncc(Cl)c(Nc5ccc6c(c5)c5c(c(=O)n6C)OCC(F)(F)C(C6CC6)N5)n3)CC4)cc21] |
| 468a | 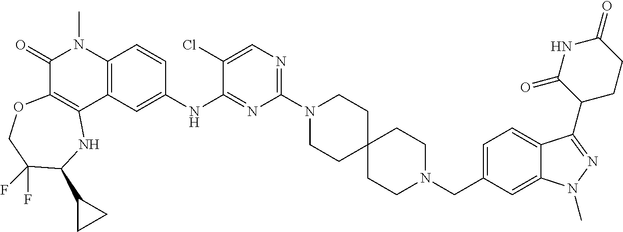 [SMILES: Cn1nc(C2CCC(=O)NC2=O)c2ccc(CN3CCC4(CC3)CCN(c3ncc(Cl)c(Nc5ccc6c(c5)c5c(c(=O)n6C)OCC(F)(F)[C@H](C6CC6)N5)n3)CC4)cc21] |
| 469 | 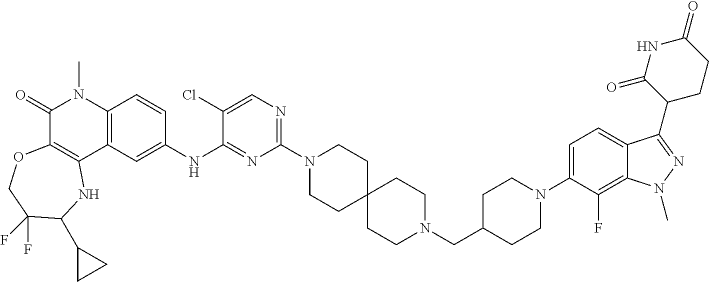 [SMILES: Cn1nc(C2CCC(=O)NC2=O)c2ccc(N3CCC(CN4CCC5(CC4)CCN(c4ncc(Cl)c(Nc6ccc7c(c6)c6c(c(=O)n7C)OCC(F)(F)C(C7CC7)N6)n4)CC5)CC3)c(F)c21] |
| 469a | 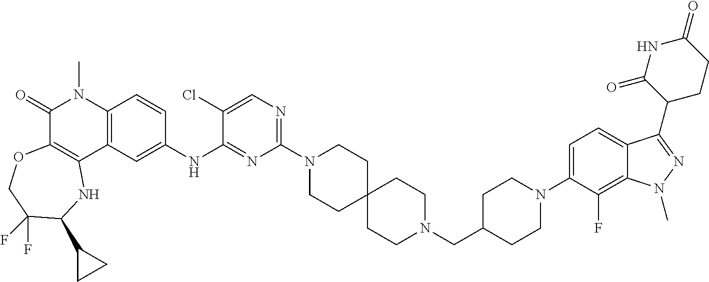 [SMILES: Cn1nc(C2CCC(=O)NC2=O)c2ccc(N3CCC(CN4CCC5(CC4)CCN(c4ncc(Cl)c(Nc6ccc7c(c6)c6c(c(=O)n7C)OCC(F)(F)[C@H](C7CC7)N6)n4)CC5)CC3)c(F)c21] |
| 470 | 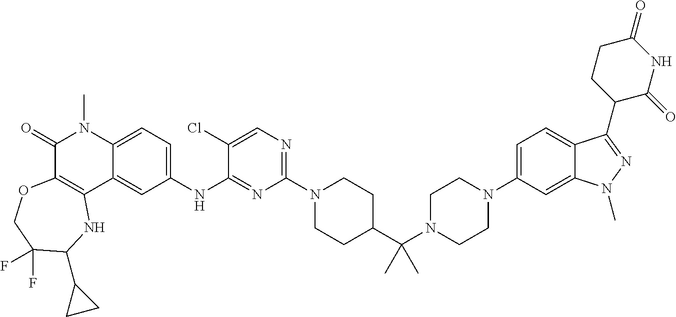 [SMILES: Cn1nc(C2CCC(=O)NC2=O)c2ccc(N3CCN(C(C)(C)C4CCN(c5ncc(Cl)c(Nc6ccc7c(c6)c6c(c(=O)n7C)OCC(F)(F)C(C7CC7)N6)n5)CC4)CC3)cc21] |
| 470a | 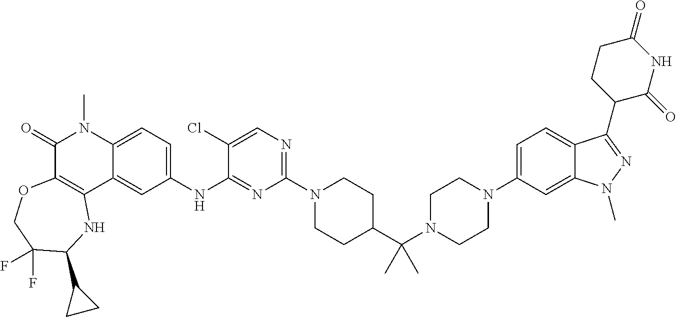 [SMILES: Cn1nc(C2CCC(=O)NC2=O)c2ccc(N3CCN(C(C)(C)C4CCN(c5ncc(Cl)c(Nc6ccc7c(c6)c6c(c(=O)n7C)OCC(F)(F)[C@H](C7CC7)N6)n5)CC4)CC3)cc21] |
| 471 | 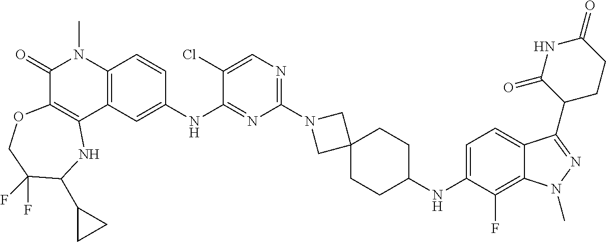 [SMILES: Cn1nc(C2CCC(=O)NC2=O)c2ccc(NC3CCC4(CC3)CN(c3ncc(Cl)c(Nc5ccc6c(c5)c5c(c(=O)n6C)OCC(F)(F)C(C6CC6)N5)n3)C4)c(F)c21] |
| 471a | 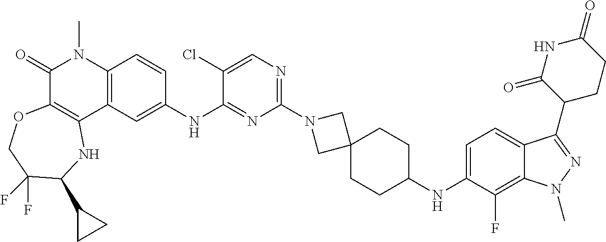 [SMILES: Cn1nc(C2CCC(=O)NC2=O)c2ccc(NC3CCC4(CC3)CN(c3ncc(Cl)c(Nc5ccc6c(c5)c5c(c(=O)n6C)OCC(F)(F)[C@H](C6CC6)N5)n3)C4)c(F)c21] |
| 472 | 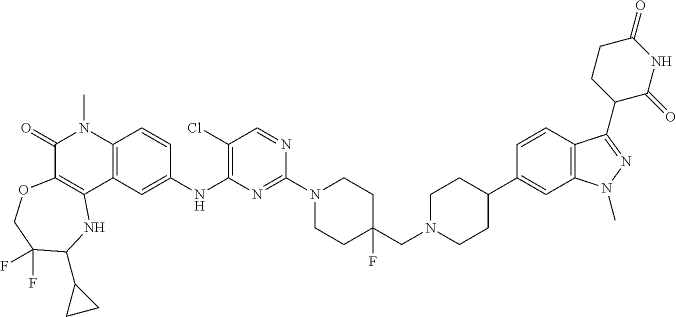 [SMILES: Cn1nc(C2CCC(=O)NC2=O)c2ccc(C3CCN(CC4(F)CCN(c5ncc(Cl)c(Nc6ccc7c(c6)c6c(c(=O)n7C)OCC(F)(F)C(C7CC7)N6)n5)CC4)CC3)cc21] |
| 472a | 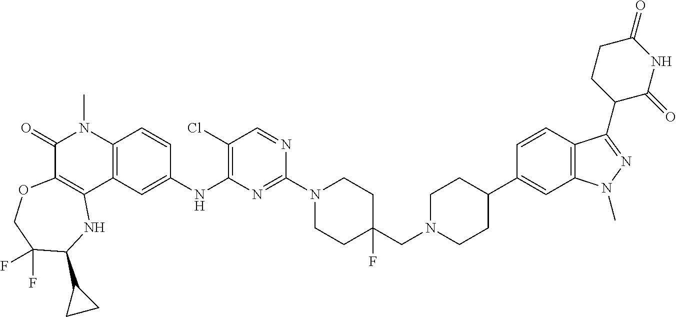 [SMILES: Cn1nc(C2CCC(=O)NC2=O)c2ccc(C3CCN(CC4(F)CCN(c5ncc(Cl)c(Nc6ccc7c(c6)c6c(c(=O)n7C)OCC(F)(F)[C@H](C7CC7)N6)n5)CC4)CC3)cc21] |
| 473 | 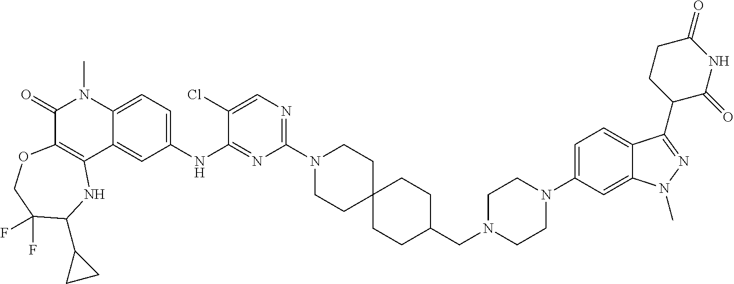 [SMILES: Cn1nc(C2CCC(=O)NC2=O)c2ccc(N3CCN(CC4CCC5(CC4)CCN(c4ncc(Cl)c(Nc6ccc7c(c6)c6c(c(=O)n7C)OCC(F)(F)C(C7CC7)N6)n4)CC5)CC3)cc21] |
| 473a | 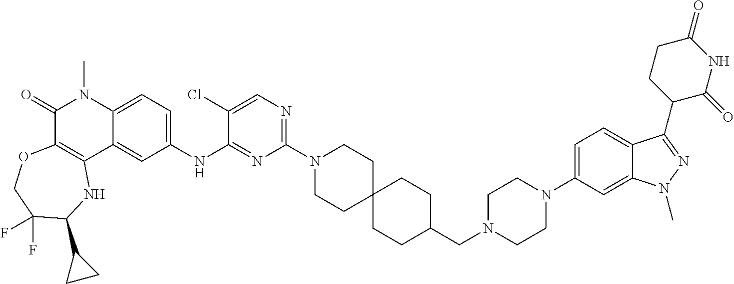 [SMILES: Cn1nc(C2CCC(=O)NC2=O)c2ccc(N3CCN(CC4CCC5(CC4)CCN(c4ncc(Cl)c(Nc6ccc7c(c6)c6c(c(=O)n7C)OCC(F)(F)[C@H](C7CC7)N6)n4)CC5)CC3)cc21] |
| 474 | 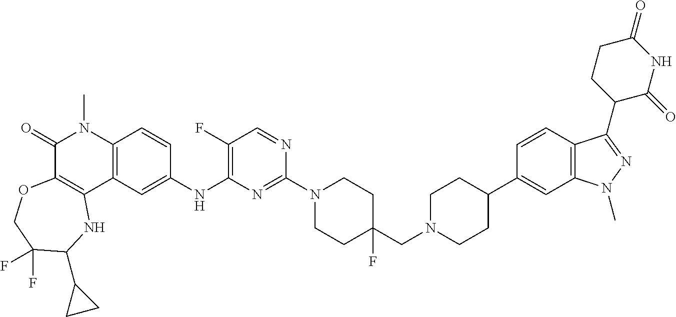 [SMILES: Cn1nc(C2CCC(=O)NC2=O)c2ccc(C3CCN(CC4(F)CCN(c5ncc(F)c(Nc6ccc7c(c6)c6c(c(=O)n7C)OCC(F)(F)C(C7CC7)N6)n5)CC4)CC3)cc21] |
| 474a | 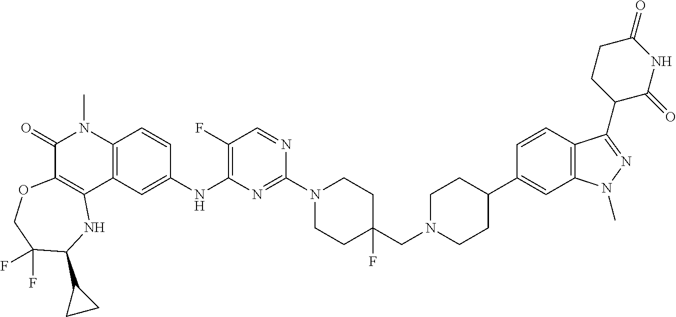 [SMILES: Cn1nc(C2CCC(=O)NC2=O)c2ccc(C3CCN(CC4(F)CCN(c5ncc(F)c(Nc6ccc7c(c6)c6c(c(=O)n7C)OCC(F)(F)[C@H](C7CC7)N6)n5)CC4)CC3)cc21] |
| 475 | 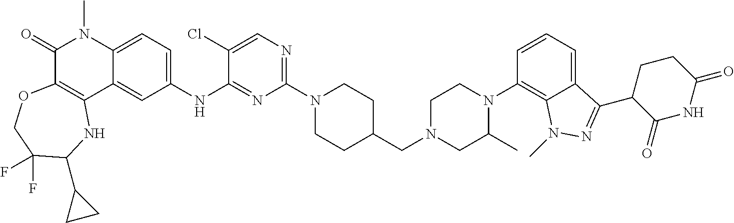 [SMILES: CC1CN(CC2CCN(c3ncc(Cl)c(Nc4ccc5c(c4)c4c(c(=O)n5C)OCC(F)(F)C(C5CC5)N4)n3)CC2)CCN1c1cccc2c(C3CCC(=O)NC3=O)nn(C)c12] |
| 475a | 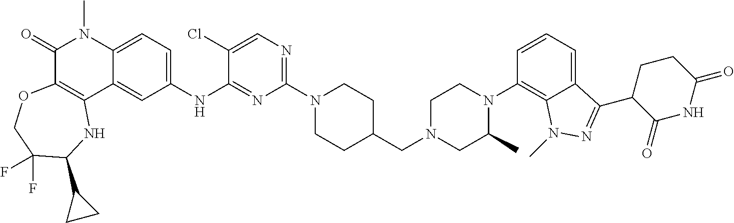 [SMILES: C[C@H]1CN(CC2CCN(c3ncc(Cl)c(Nc4ccc5c(c4)c4c(c(=O)n5C)OCC(F)(F)[C@H](C5CC5)N4)n3)CC2)CCN1c1cccc2c(C3CCC(=O)NC3=O)nn(C)c12] |
| 475b | 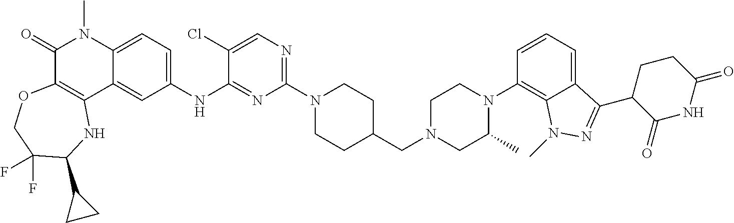 [SMILES: C[C@@H]1CN(CC2CCN(c3ncc(Cl)c(Nc4ccc5c(c4)c4c(c(=O)n5C)OCC(F)(F)[C@H](C5CC5)N4)n3)CC2)CCN1c1cccc2c(C3CCC(=O)NC3=O)nn(C)c12] |
| 476 | 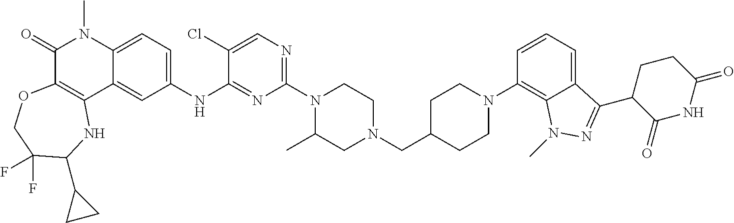 [SMILES: CC1CN(CC2CCN(c3cccc4c(C5CCC(=O)NC5=O)nn(C)c34)CC2)CCN1c1ncc(Cl)c(Nc2ccc3c(c2)c2c(c(=O)n3C)OCC(F)(F)C(C3CC3)N2)n1] |
| 476a | 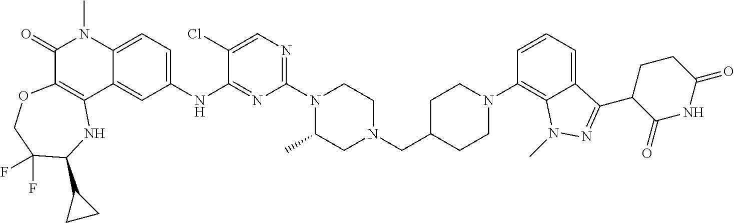 [SMILES: C[C@H]1CN(CC2CCN(c3cccc4c(C5CCC(=O)NC5=O)nn(C)c34)CC2)CCN1c1ncc(Cl)c(Nc2ccc3c(c2)c2c(c(=O)n3C)OCC(F)(F)[C@H](C3CC3)N2)n1] |
| 476b | 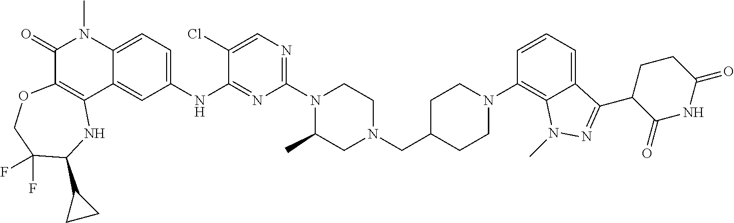 [SMILES: C[C@@H]1CN(CC2CCN(c3cccc4c(C5CCC(=O)NC5=O)nn(C)c34)CC2)CCN1c1ncc(Cl)c(Nc2ccc3c(c2)c2c(c(=O)n3C)OCC(F)(F)[C@H](C3CC3)N2)n1] |
| 477 | 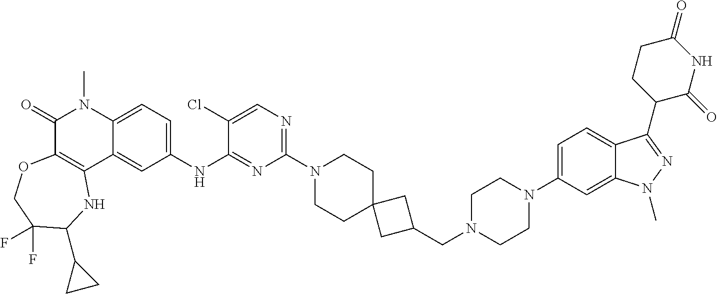 [SMILES: Cn1nc(C2CCC(=O)NC2=O)c2ccc(N3CCN(CC4CC5(CCN(c6ncc(Cl)c(Nc7ccc8c(c7)c7c(c(=O)n8C)OCC(F)(F)C(C8CC8)N7)n6)CC5)C4)CC3)cc21] |
| 477a | 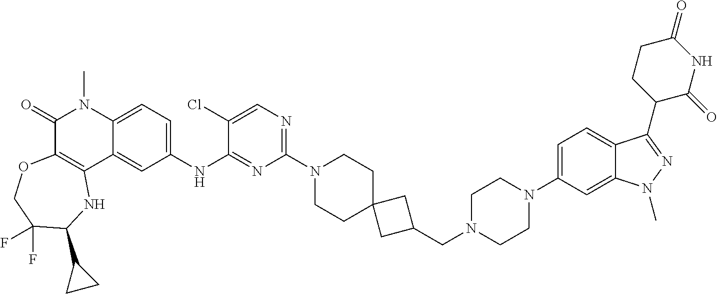 [SMILES: Cn1nc(C2CCC(=O)NC2=O)c2ccc(N3CCN(CC4CC5(CCN(c6ncc(Cl)c(Nc7ccc8c(c7)c7c(c(=O)n8C)OCC(F)(F)[C@H](C8CC8)N7)n6)CC5)C4)CC3)cc21] |
| 478 | 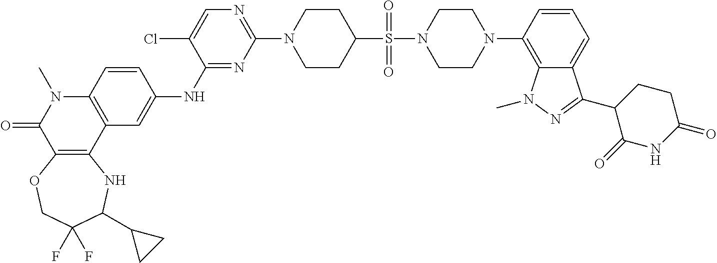 [SMILES: Cn1nc(C2CCC(=O)NC2=O)c2cccc(N3CCN(S(=O)(=O)C4CCN(c5ncc(Cl)c(Nc6ccc7c(c6)c6c(c(=O)n7C)OCC(F)(F)C(C7CC7)N6)n5)CC4)CC3)c21] |
| 478a | 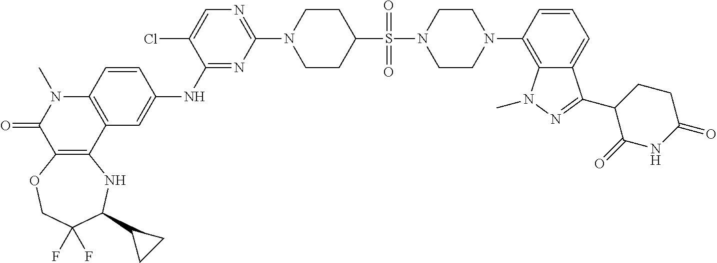 [SMILES: Cn1nc(C2CCC(=O)NC2=O)c2cccc(N3CCN(S(=O)(=O)C4CCN(c5ncc(Cl)c(Nc6ccc7c(c6)c6c(c(=O)n7C)OCC(F)(F)[C@H](C7CC7)N6)n5)CC4)CC3)c21] |
| 479 | 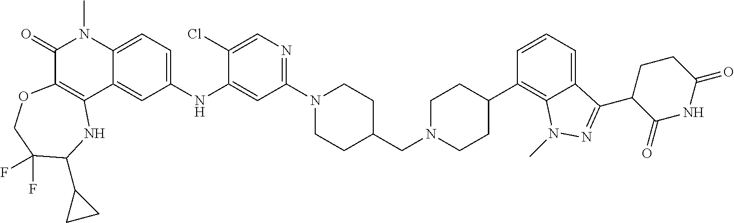 [SMILES: Cn1nc(C2CCC(=O)NC2=O)c2cccc(C3CCN(CC4CCN(c5cc(Nc6ccc7c(c6)c6c(c(=O)n7C)OCC(F)(F)C(C7CC7)N6)c(Cl)cn5)CC4)CC3)c21] |
| 479a | 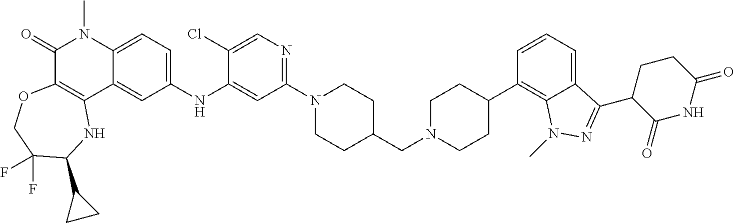 [SMILES: Cn1nc(C2CCC(=O)NC2=O)c2cccc(C3CCN(CC4CCN(c5cc(Nc6ccc7c(c6)c6c(c(=O)n7C)OCC(F)(F)[C@H](C7CC7)N6)c(Cl)cn5)CC4)CC3)c21] |
| 480 | 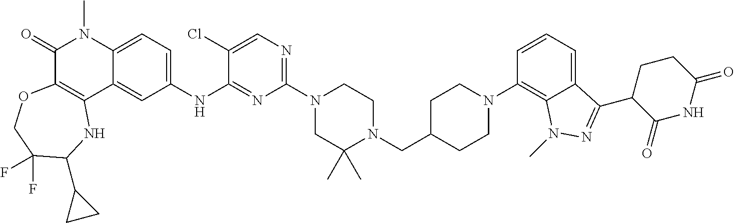 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)CC4(C)C)CC3)c21] |
| 480a | 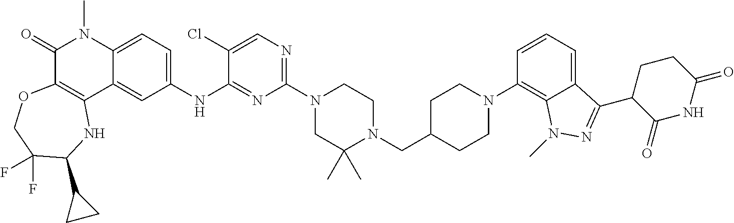 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)CC4(C)C)CC3)c21] |
| 481 | 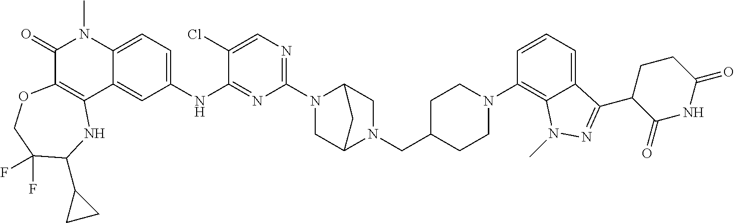 [SMILES: Cn1nc(C2CCC(=O)NC2=O)c2cccc(N3CCC(CN4CC5CC4CN5c4ncc(Cl)c(Nc5ccc6c(c5)c5c(c(=O)n6C)OCC(F)(F)C(C6CC6)N5)n4)CC3)c21] |
| 481a | 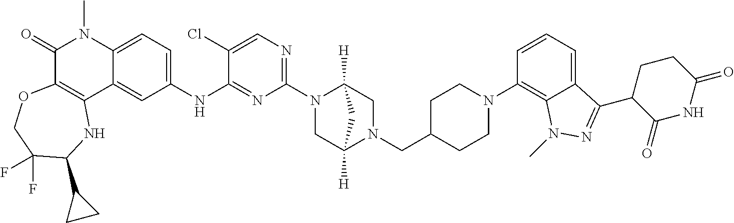 [SMILES: Cn1nc(C2CCC(=O)NC2=O)c2cccc(N3CCC(CN4C[C@H]5C[C@@H]4CN5c4ncc(Cl)c(Nc5ccc6c(c5)c5c(c(=O)n6C)OCC(F)(F)[C@H](C6CC6)N5)n4)CC3)c21] |
| 481b | 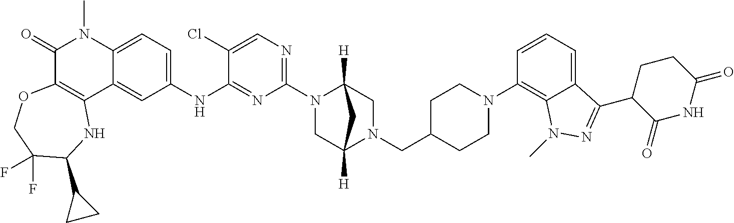 [SMILES: Cn1nc(C2CCC(=O)NC2=O)c2cccc(N3CCC(CN4C[C@@H]5C[C@H]4CN5c4ncc(Cl)c(Nc5ccc6c(c5)c5c(c(=O)n6C)OCC(F)(F)[C@H](C6CC6)N5)n4)CC3)c21] |
| 482 | 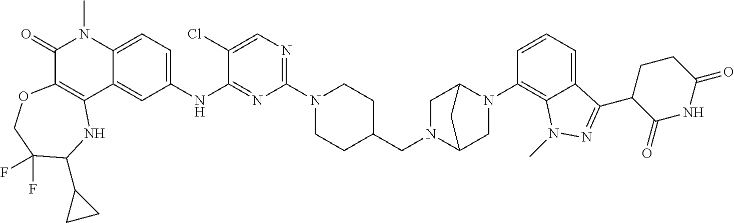 [SMILES: Cn1nc(C2CCC(=O)NC2=O)c2cccc(N3CC4CC3CN4CC3CCN(c4ncc(Cl)c(Nc5ccc6c(c5)c5c(c(=O)n6C)OCC(F)(F)C(C6CC6)N5)n4)CC3)c21] |
| 482a | 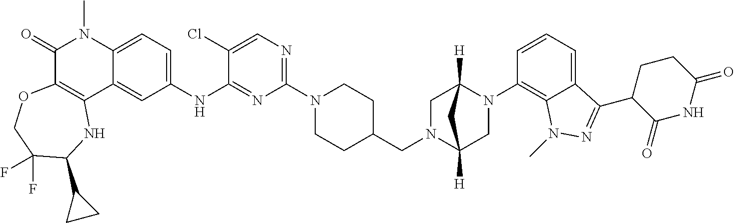 [SMILES: Cn1nc(C2CCC(=O)NC2=O)c2cccc(N3C[C@H]4C[C@@H]3CN4CC3CCN(c4ncc(Cl)c(Nc5ccc6c(c5)c5c(c(=O)n6C)OCC(F)(F)[C@H](C6CC6)N5)n4)CC3)c21] |
| 482b | 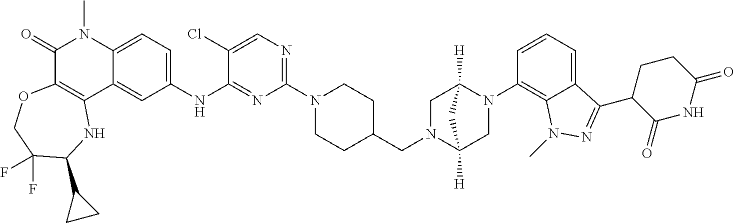 [SMILES: Cn1nc(C2CCC(=O)NC2=O)c2cccc(N3C[C@@H]4C[C@H]3CN4CC3CCN(c4ncc(Cl)c(Nc5ccc6c(c5)c5c(c(=O)n6C)OCC(F)(F)[C@H](C6CC6)N5)n4)CC3)c21] |
| 483 | 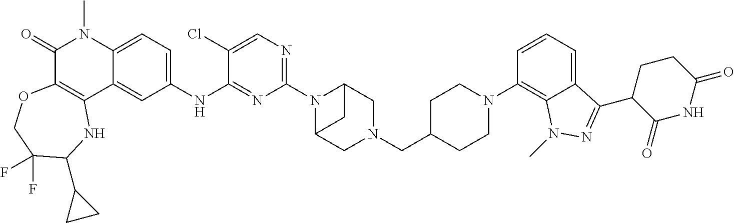 [SMILES: Cn1nc(C2CCC(=O)NC2=O)c2cccc(N3CCC(CN4CC5CC(C4)N5c4ncc(Cl)c(Nc5ccc6c(c5)c5c(c(=O)n6C)OCC(F)(F)C(C6CC6)N5)n4)CC3)c21] |
| 483a | 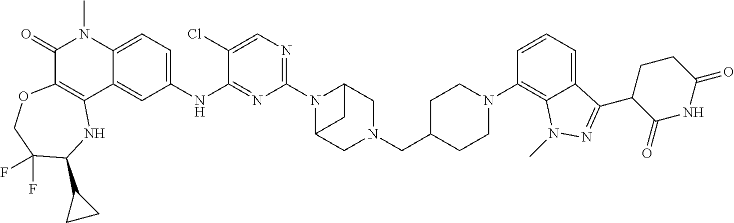 [SMILES: Cn1nc(C2CCC(=O)NC2=O)c2cccc(N3CCC(CN4CC5CC(C4)N5c4ncc(Cl)c(Nc5ccc6c(c5)c5c(c(=O)n6C)OCC(F)(F)[C@H](C6CC6)N5)n4)CC3)c21] |
| 484 | 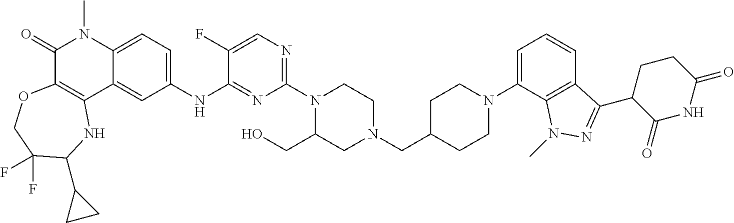 [SMILES: Cn1nc(C2CCC(=O)NC2=O)c2cccc(N3CCC(CN4CCN(c5ncc(F)c(Nc6ccc7c(c6)c6c(c(=O)n7C)OCC(F)(F)C(C7CC7)N6)n5)C(CO)C4)CC3)c21] |
| 484a | 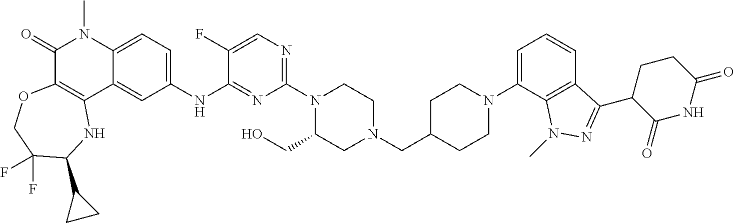 [SMILES: Cn1nc(C2CCC(=O)NC2=O)c2cccc(N3CCC(CN4CCN(c5ncc(F)c(Nc6ccc7c(c6)c6c(c(=O)n7C)OCC(F)(F)[C@H](C7CC7)N6)n5)[C@@H](CO)C4)CC3)c21] |
| 485 | 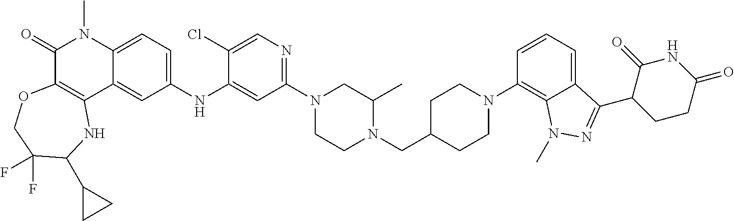 [SMILES: CC1CN(c2cc(Nc3ccc4c(c3)c3c(c(=O)n4C)OCC(F)(F)C(C4CC4)N3)c(Cl)cn2)CCN1CC1CCN(c2cccc3c(C4CCC(=O)NC4=O)nn(C)c23)CC1] |
| 485a | 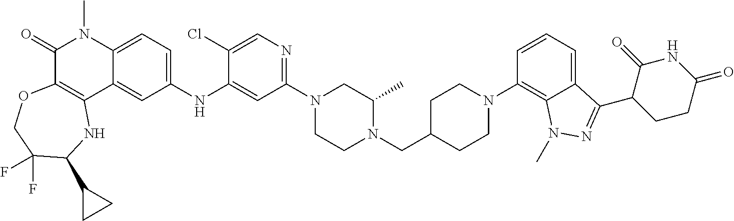 [SMILES: C[C@H]1CN(c2cc(Nc3ccc4c(c3)c3c(c(=O)n4C)OCC(F)(F)[C@H](C4CC4)N3)c(Cl)cn2)CCN1CC1CCN(c2cccc3c(C4CCC(=O)NC4=O)nn(C)c23)CC1] |
| 486 | 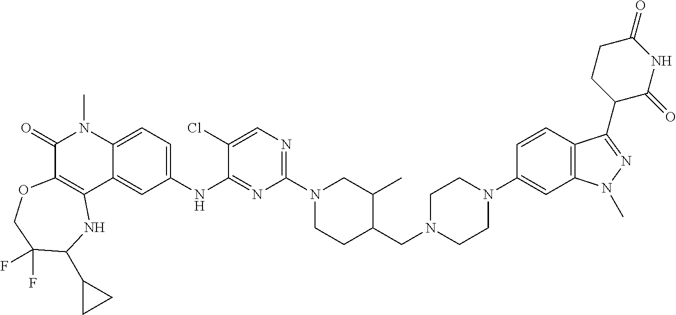 [SMILES: CC1CN(c2ncc(Cl)c(Nc3ccc4c(c3)c3c(c(=O)n4C)OCC(F)(F)C(C4CC4)N3)n2)CCC1CN1CCN(c2ccc3c(C4CCC(=O)NC4=O)nn(C)c3c2)CC1] |
| 486a | 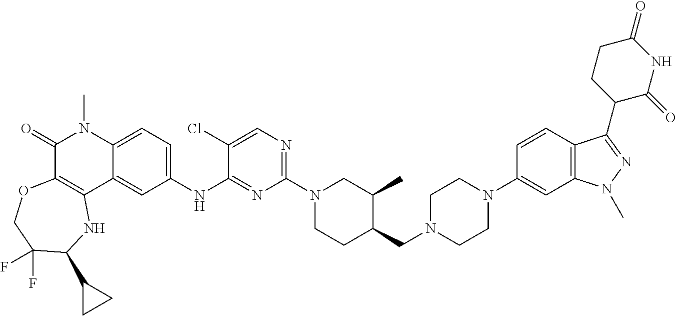 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCN(c2ccc3c(C4CCC(=O)NC4=O)nn(C)c3c2)CC1] |
| 486b | 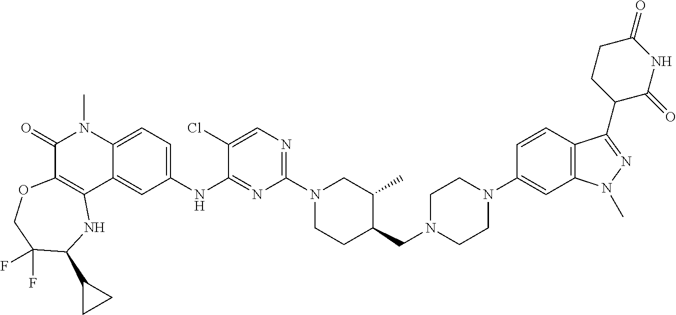 [SMILES: C[C@H]1CN(c2ncc(Cl)c(Nc3ccc4c(c3)c3c(c(=O)n4C)OCC(F)(F)[C@H](C4CC4)N3)n2)CC[C@@H]1CN1CCN(c2ccc3c(C4CCC(=O)NC4=O)nn(C)c3c2)CC1] |
| 486c | 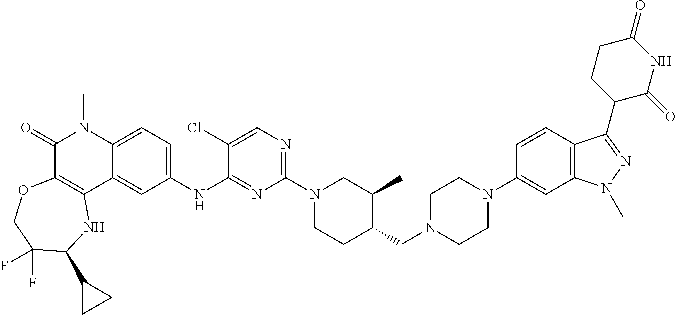 [SMILES: C[C@@H]1CN(c2ncc(Cl)c(Nc3ccc4c(c3)c3c(c(=O)n4C)OCC(F)(F)[C@H](C4CC4)N3)n2)CC[C@H]1CN1CCN(c2ccc3c(C4CCC(=O)NC4=O)nn(C)c3c2)CC1] |
| 486d | 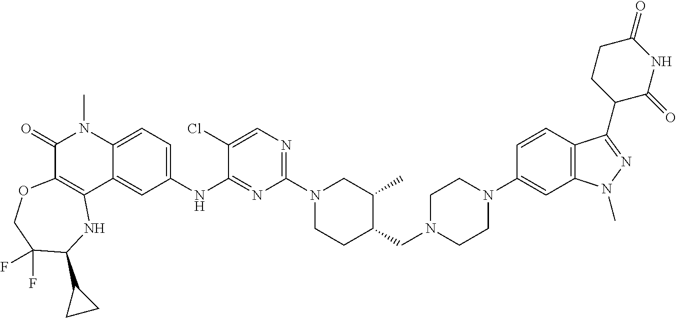 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCN(c2ccc3c(C4CCC(=O)NC4=O)nn(C)c3c2)CC1] |
| 487 | 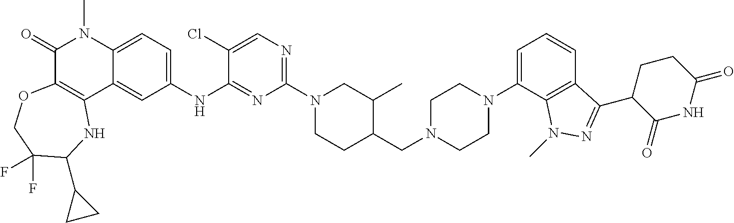 [SMILES: CC1CN(c2ncc(Cl)c(Nc3ccc4c(c3)c3c(c(=O)n4C)OCC(F)(F)C(C4CC4)N3)n2)CCC1CN1CCN(c2cccc3c(C4CCC(=O)NC4=O)nn(C)c23)CC1] |
| 487a | 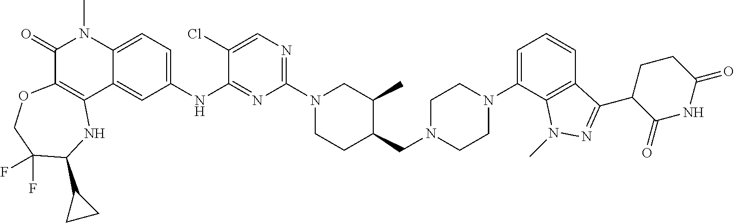 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCN(c2cccc3c(C4CCC(=O)NC4=O)nn(C)c23)CC1] |
| 487b | 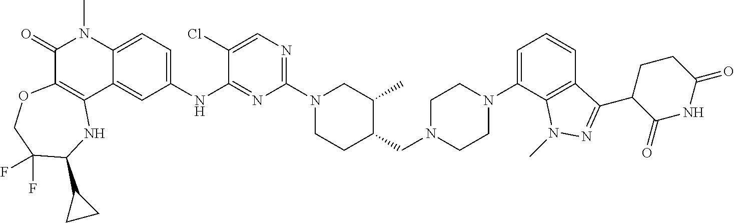 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCN(c2cccc3c(C4CCC(=O)NC4=O)nn(C)c23)CC1] |
| 487c | 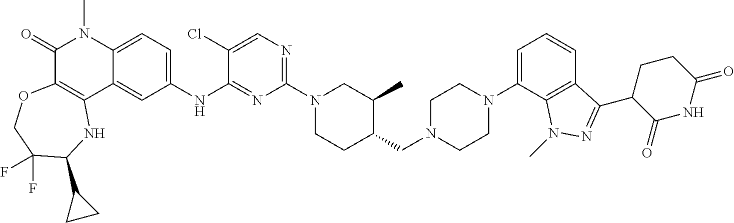 [SMILES: C[C@@H]1CN(c2ncc(Cl)c(Nc3ccc4c(c3)c3c(c(=O)n4C)OCC(F)(F)[C@H](C4CC4)N3)n2)CC[C@H]1CN1CCN(c2cccc3c(C4CCC(=O)NC4=O)nn(C)c23)CC1] |
| 487d | 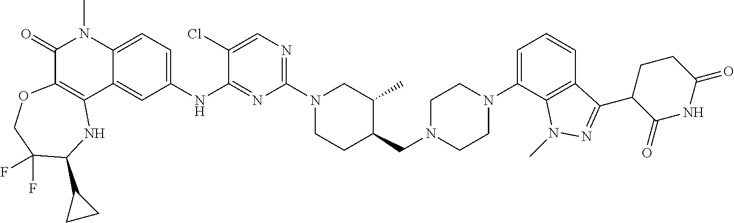 [SMILES: C[C@H]1CN(c2ncc(Cl)c(Nc3ccc4c(c3)c3c(c(=O)n4C)OCC(F)(F)[C@H](C4CC4)N3)n2)CC[C@@H]1CN1CCN(c2cccc3c(C4CCC(=O)NC4=O)nn(C)c23)CC1] |
| 488 | 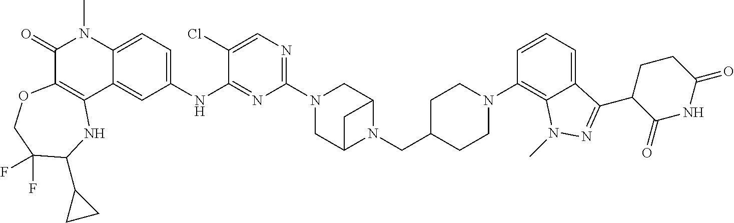 [SMILES: Cn1nc(C2CCC(=O)NC2=O)c2cccc(N3CCC(CN4C5CC4CN(c4ncc(Cl)c(Nc6ccc7c(c6)c6c(c(=O)n7C)OCC(F)(F)C(C7CC7)N6)n4)C5)CC3)c21] |
| 488a | 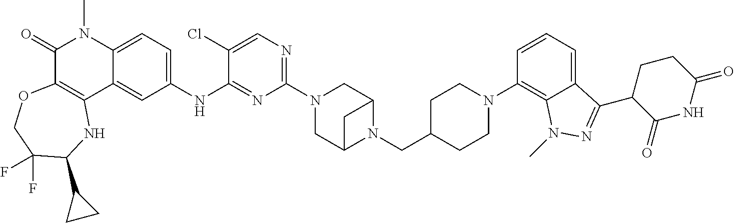 [SMILES: Cn1nc(C2CCC(=O)NC2=O)c2cccc(N3CCC(CN4C5CC4CN(c4ncc(Cl)c(Nc6ccc7c(c6)c6c(c(=O)n7C)OCC(F)(F)[C@H](C7CC7)N6)n4)C5)CC3)c21] |
| 489 | 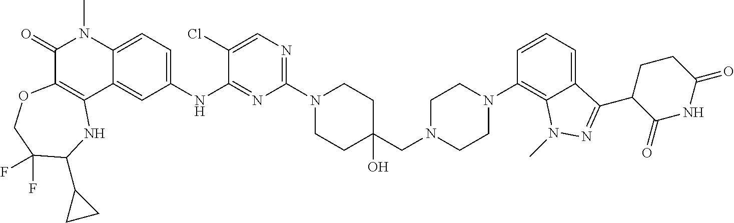 [SMILES: Cn1nc(C2CCC(=O)NC2=O)c2cccc(N3CCN(CC4(O)CCN(c5ncc(Cl)c(Nc6ccc7c(c6)c6c(c(=O)n7C)OCC(F)(F)C(C7CC7)N6)n5)CC4)CC3)c21] |
| 489a | 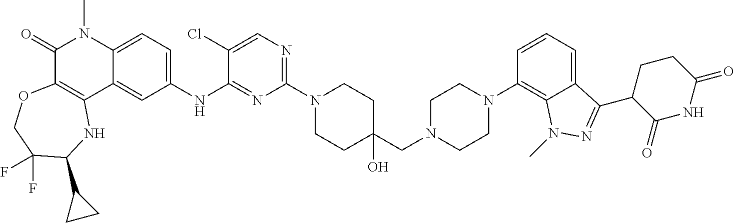 [SMILES: Cn1nc(C2CCC(=O)NC2=O)c2cccc(N3CCN(CC4(O)CCN(c5ncc(Cl)c(Nc6ccc7c(c6)c6c(c(=O)n7C)OCC(F)(F)[C@H](C7CC7)N6)n5)CC4)CC3)c21] |
| 490 | 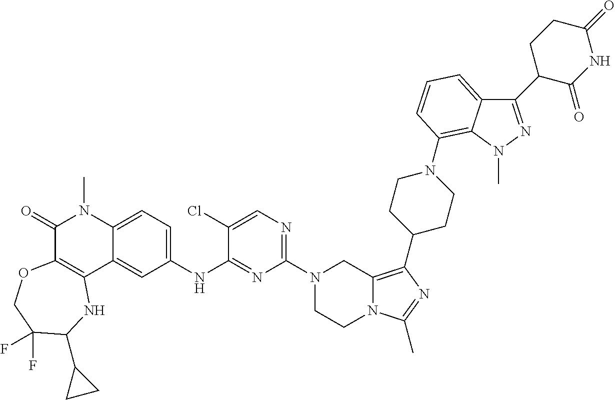 [SMILES: Cc1nc(C2CCN(c3cccc4c(C5CCC(=O)NC5=O)nn(C)c34)CC2)c2n1CCN(c1ncc(Cl)c(Nc3ccc4c(c3)c3c(c(=O)n4C)OCC(F)(F)C(C4CC4)N3)n1)C2] |
| 490a | 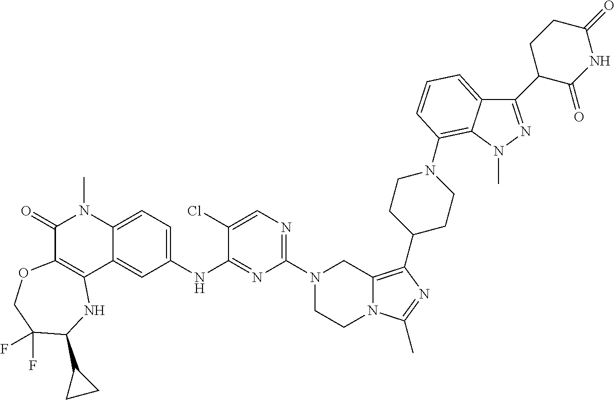 [SMILES: Cc1nc(C2CCN(c3cccc4c(C5CCC(=O)NC5=O)nn(C)c34)CC2)c2n1CCN(c1ncc(Cl)c(Nc3ccc4c(c3)c3c(c(=O)n4C)OCC(F)(F)[C@H](C4CC4)N3)n1)C2] |
| 491 | 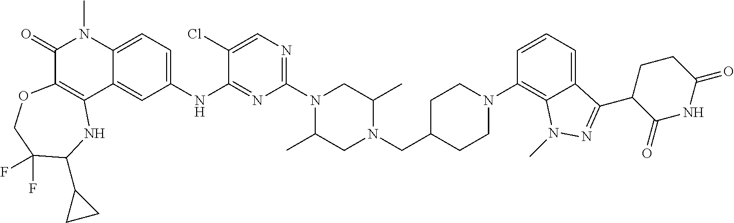 [SMILES: CC1CN(c2ncc(Cl)c(Nc3ccc4c(c3)c3c(c(=O)n4C)OCC(F)(F)C(C4CC4)N3)n2)C(C)CN1CC1CCN(c2cccc3c(C4CCC(=O)NC4=O)nn(C)c23)CC1] |
| 491a | 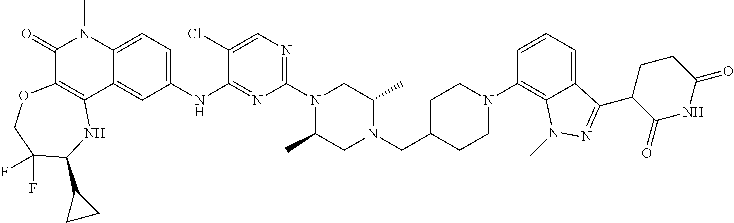 [SMILES: C[C@@H]1CN(CC2CCN(c3cccc4c(C5CCC(=O)NC5=O)nn(C)c34)CC2)[C@@H](C)CN1c1ncc(Cl)c(Nc2ccc3c(c2)c2c(c(=O)n3C)OCC(F)(F)[C@H](C3CC3)N2)n1] |
| 491b | 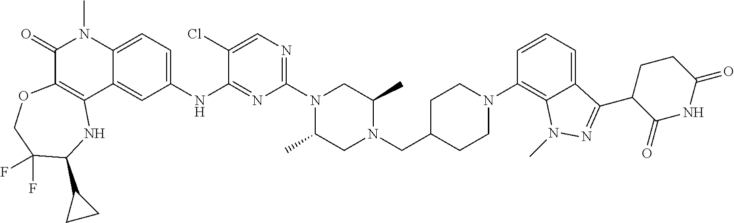 [SMILES: C[C@@H]1CN(c2ncc(Cl)c(Nc3ccc4c(c3)c3c(c(=O)n4C)OCC(F)(F)[C@H](C4CC4)N3)n2)[C@@H](C)CN1CC1CCN(c2cccc3c(C4CCC(=O)NC4=O)nn(C)c23)CC1] |
| 492 | 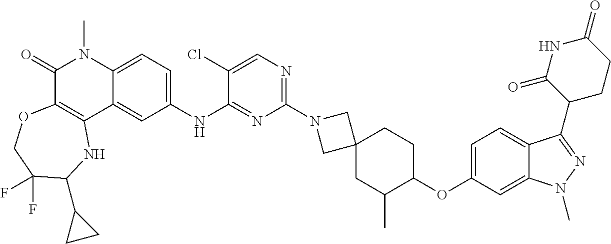 [SMILES: CC1CC2(CCC1Oc1ccc3c(C4CCC(=O)NC4=O)nn(C)c3c1)CN(c1ncc(Cl)c(Nc3ccc4c(c3)c3c(c(=O)n4C)OCC(F)(F)C(C4CC4)N3)n1)C2] |
| 492a | 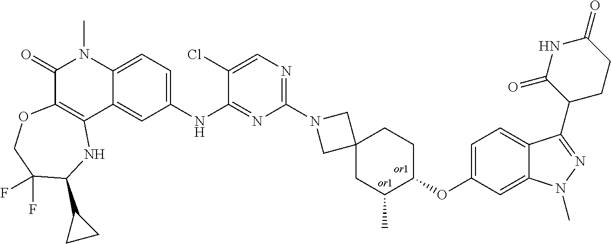 [SMILES: C[C@@H]1CC2(CC[C@@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 492b | 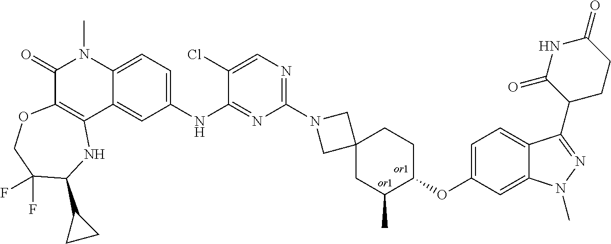 [SMILES: C[C@H]1CC2(CC[C@@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 492c | 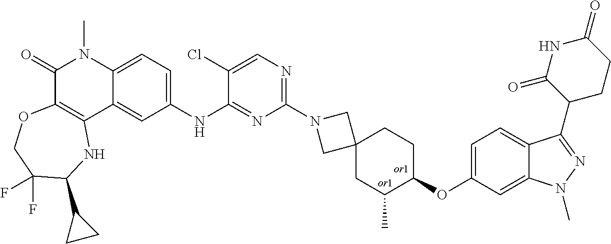 [SMILES: C[C@@H]1CC2(CC[C@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 492d | 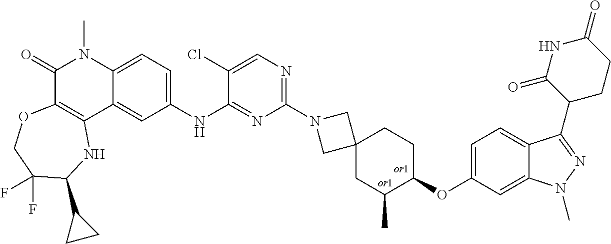 [SMILES: C[C@H]1CC2(CC[C@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 493 | 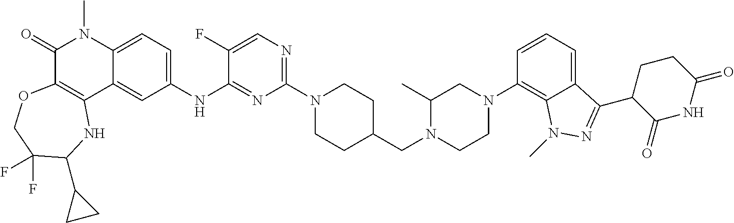 [SMILES: CC1CN(c2cccc3c(C4CCC(=O)NC4=O)nn(C)c23)CCN1CC1CCN(c2ncc(F)c(Nc3ccc4c(c3)c3c(c(=O)n4C)OCC(F)(F)C(C4CC4)N3)n2)CC1] |
| 493a | 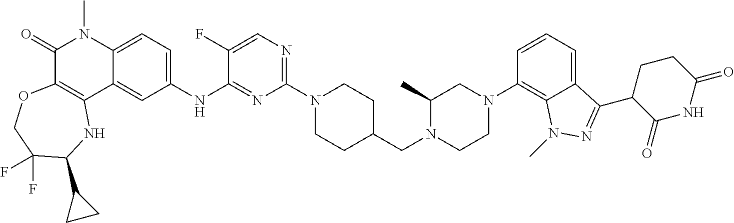 [SMILES: C[C@H]1CN(c2cccc3c(C4CCC(=O)NC4=O)nn(C)c23)CCN1CC1CCN(c2ncc(F)c(Nc3ccc4c(c3)c3c(c(=O)n4C)OCC(F)(F)[C@H](C4CC4)N3)n2)CC1] |
| 493b | 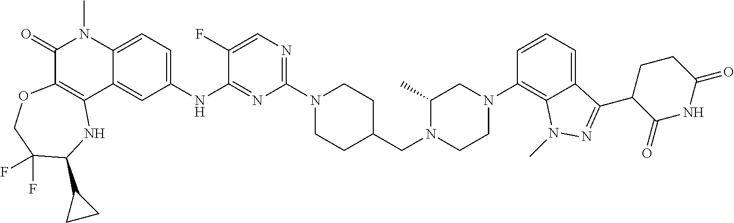 [SMILES: C[C@@H]1CN(c2cccc3c(C4CCC(=O)NC4=O)nn(C)c23)CCN1CC1CCN(c2ncc(F)c(Nc3ccc4c(c3)c3c(c(=O)n4C)OCC(F)(F)[C@H](C4CC4)N3)n2)CC1] |
| 494 | 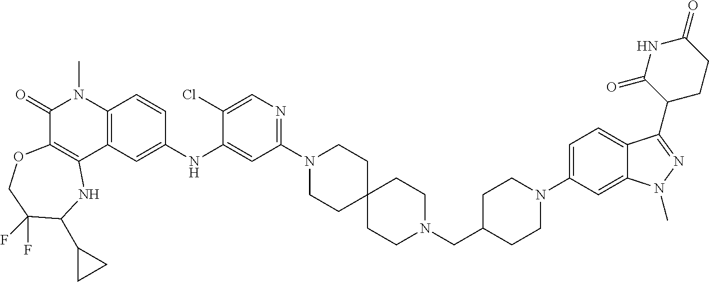 [SMILES: Cn1nc(C2CCC(=O)NC2=O)c2ccc(N3CCC(CN4CCC5(CC4)CCN(c4cc(Nc6ccc7c(c6)c6c(c(=O)n7C)OCC(F)(F)C(C7CC7)N6)c(Cl)cn4)CC5)CC3)cc21] |
| 494a | 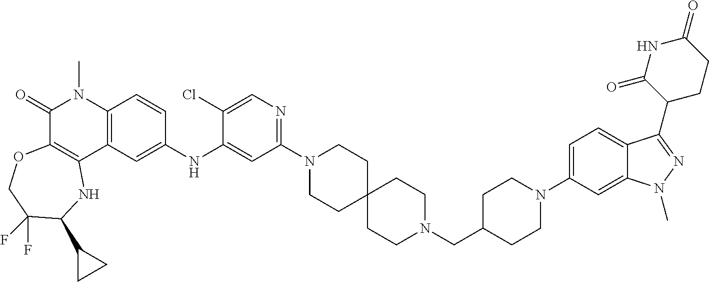 [SMILES: Cn1nc(C2CCC(=O)NC2=O)c2ccc(N3CCC(CN4CCC5(CC4)CCN(c4cc(Nc6ccc7c(c6)c6c(c(=O)n7C)OCC(F)(F)[C@H](C7CC7)N6)c(Cl)cn4)CC5)CC3)cc21] |
| 495 | 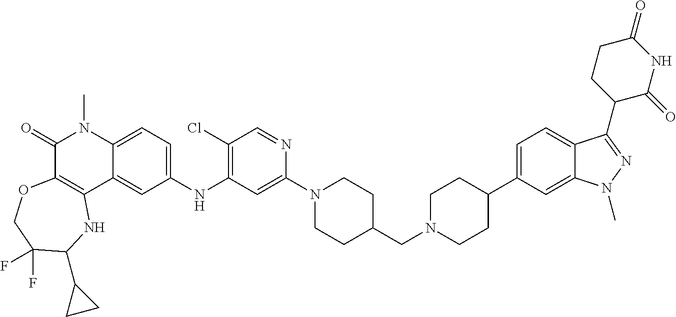 [SMILES: Cn1nc(C2CCC(=O)NC2=O)c2ccc(C3CCN(CC4CCN(c5cc(Nc6ccc7c(c6)c6c(c(=O)n7C)OCC(F)(F)C(C7CC7)N6)c(Cl)cn5)CC4)CC3)cc21] |
| 495a | 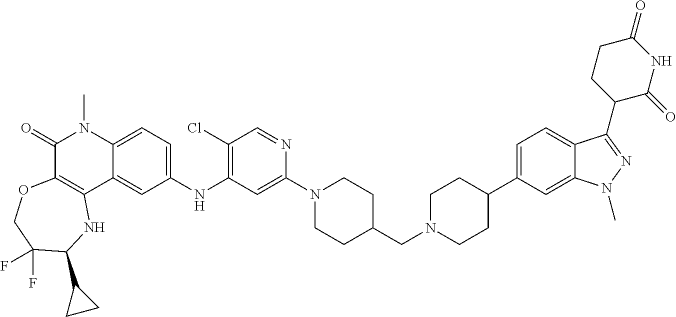 [SMILES: Cn1nc(C2CCC(=O)NC2=O)c2ccc(C3CCN(CC4CCN(c5cc(Nc6ccc7c(c6)c6c(c(=O)n7C)OCC(F)(F)[C@H](C7CC7)N6)c(Cl)cn5)CC4)CC3)cc21] |
| 496 | 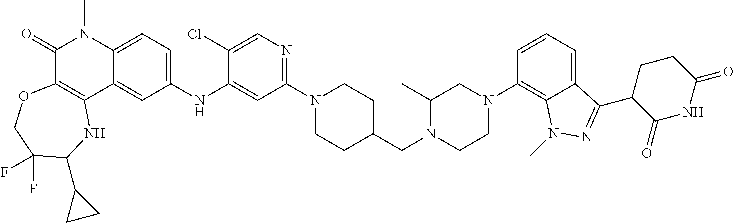 [SMILES: CC1CN(c2cccc3c(C4CCC(=O)NC4=O)nn(C)c23)CCN1CC1CCN(c2cc(Nc3ccc4c(c3)c3c(c(=O)n4C)OCC(F)(F)C(C4CC4)N3)c(Cl)cn2)CC1] |
| 496a | 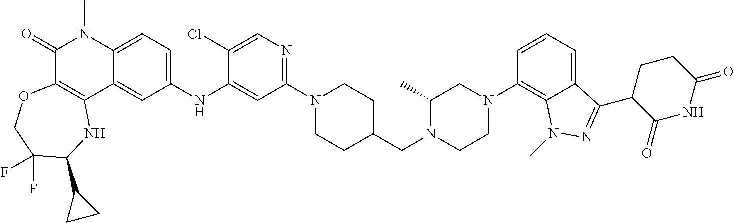 [SMILES: C[C@@H]1CN(c2cccc3c(C4CCC(=O)NC4=O)nn(C)c23)CCN1CC1CCN(c2cc(Nc3ccc4c(c3)c3c(c(=O)n4C)OCC(F)(F)[C@H](C4CC4)N3)c(Cl)cn2)CC1] |
| 496b | 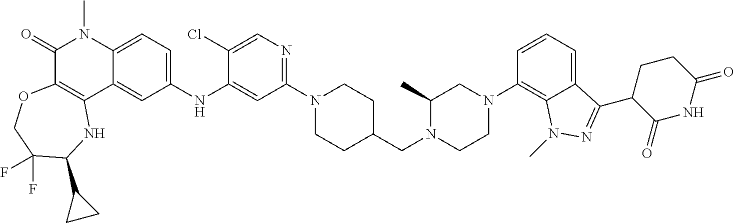 [SMILES: C[C@H]1CN(c2cccc3c(C4CCC(=O)NC4=O)nn(C)c23)CCN1CC1CCN(c2cc(Nc3ccc4c(c3)c3c(c(=O)n4C)OCC(F)(F)[C@H](C4CC4)N3)c(Cl)cn2)CC1] |
| 497 | 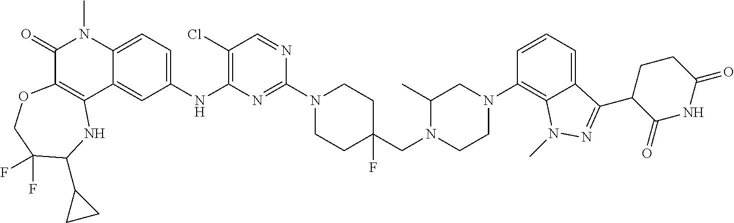 [SMILES: CC1CN(c2cccc3c(C4CCC(=O)NC4=O)nn(C)c23)CCN1CC1(F)CCN(c2ncc(Cl)c(Nc3ccc4c(c3)c3c(c(=O)n4C)OCC(F)(F)C(C4CC4)N3)n2)CC1] |
| 497a | 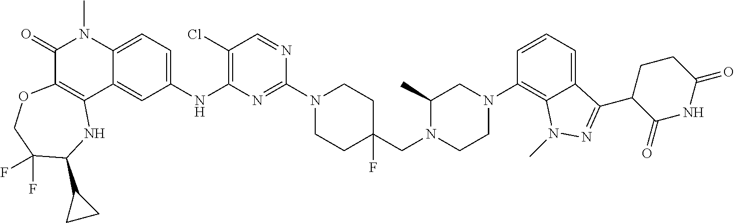 [SMILES: C[C@H]1CN(c2cccc3c(C4CCC(=O)NC4=O)nn(C)c23)CCN1CC1(F)CCN(c2ncc(Cl)c(Nc3ccc4c(c3)c3c(c(=O)n4C)OCC(F)(F)[C@H](C4CC4)N3)n2)CC1] |
| 498 | 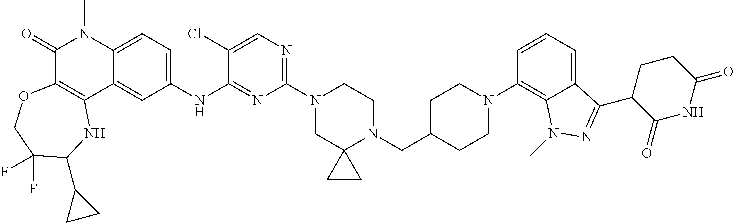 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)CC45CC5)CC3)c21] |
| 498a | 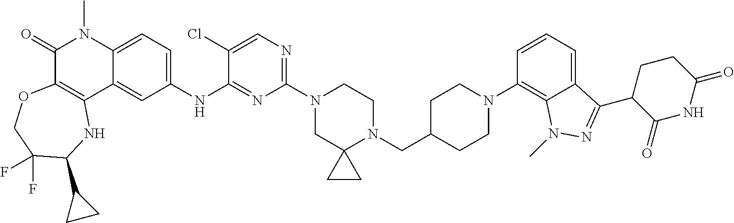 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)CC45CC5)CC3)c21] |
| 499 | 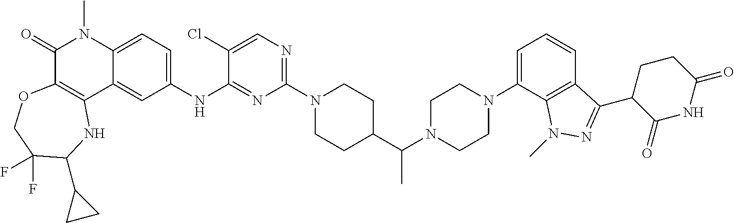 [SMILES: CC(C1CCN(c2ncc(Cl)c(Nc3ccc4c(c3)c3c(c(=O)n4C)OCC(F)(F)C(C4CC4)N3)n2)CC1)N1CCN(c2cccc3c(C4CCC(=O)NC4=O)nn(C)c23)CC1] |
| 499a | 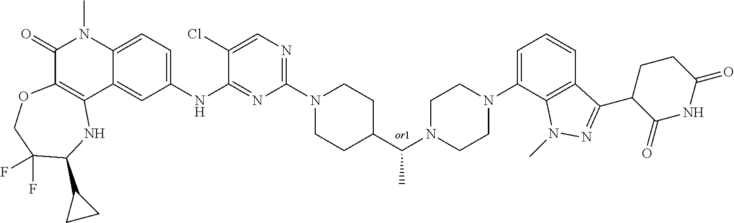 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCN(c2cccc3c(C4CCC(=O)NC4=O)nn(C)c23)CC1] |
| 499b | 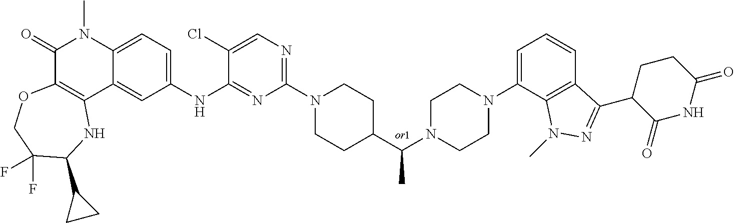 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCN(c2cccc3c(C4CCC(=O)NC4=O)nn(C)c23)CC1] |
| 500 | 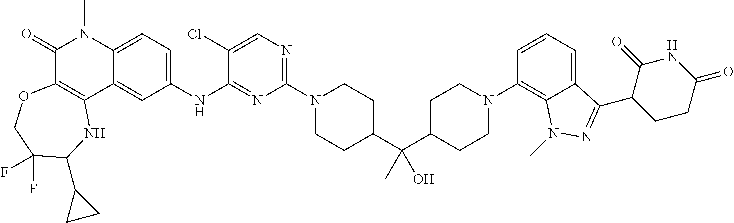 [SMILES: Cn1nc(C2CCC(=O)NC2=O)c2cccc(N3CCC(C(C)(O)C4CCN(c5ncc(Cl)c(Nc6ccc7c(c6)c6c(c(=O)n7C)OCC(F)(F)C(C7CC7)N6)n5)CC4)CC3)c21] |
| 500a | 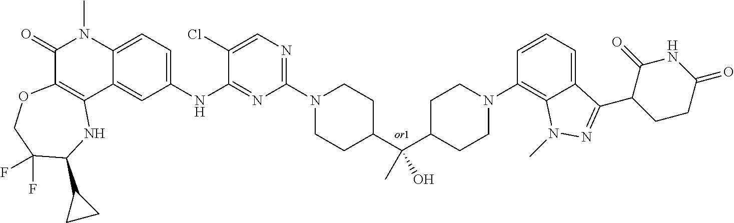 [SMILES: Cn1nc(C2CCC(=O)NC2=O)c2cccc(N3CCC([C@](C)(O)C4CCN(c5ncc(Cl)c(Nc6ccc7c(c6)c6c(c(=O)n7C)OCC(F)(F)[C@H](C7CC7)N6)n5)CC4)CC3)c21] |
| 500b | 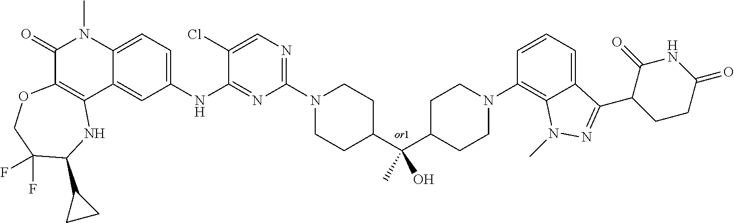 [SMILES: Cn1nc(C2CCC(=O)NC2=O)c2cccc(N3CCC([C@@](C)(O)C4CCN(c5ncc(Cl)c(Nc6ccc7c(c6)c6c(c(=O)n7C)OCC(F)(F)[C@H](C7CC7)N6)n5)CC4)CC3)c21] |
| 501 | 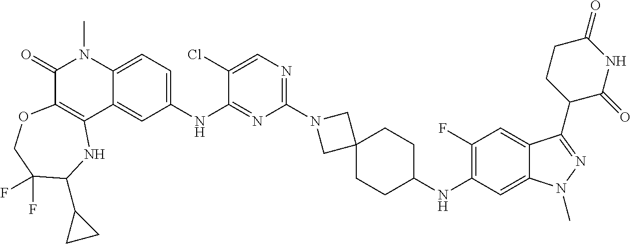 [SMILES: Cn1nc(C2CCC(=O)NC2=O)c2cc(F)c(NC3CCC4(CC3)CN(c3ncc(Cl)c(Nc5ccc6c(c5)c5c(c(=O)n6C)OCC(F)(F)C(C6CC6)N5)n3)C4)cc21] |
| 501a | 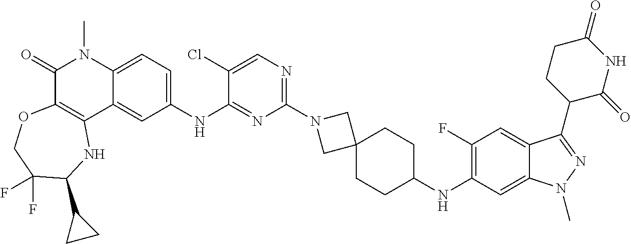 [SMILES: Cn1nc(C2CCC(=O)NC2=O)c2cc(F)c(NC3CCC4(CC3)CN(c3ncc(Cl)c(Nc5ccc6c(c5)c5c(c(=O)n6C)OCC(F)(F)[C@H](C6CC6)N5)n3)C4)cc21] |
| 502 | 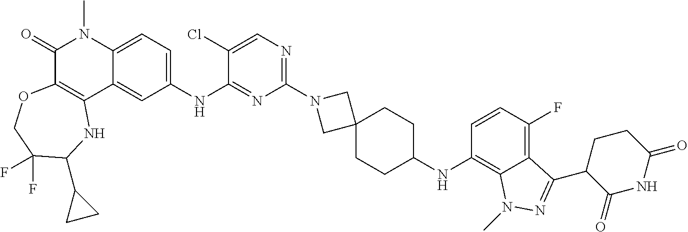 [SMILES: Cn1nc(C2CCC(=O)NC2=O)c2c(F)ccc(NC3CCC4(CC3)CN(c3ncc(Cl)c(Nc5ccc6c(c5)c5c(c(=O)n6C)OCC(F)(F)C(C6CC6)N5)n3)C4)c21] |
| 502a | 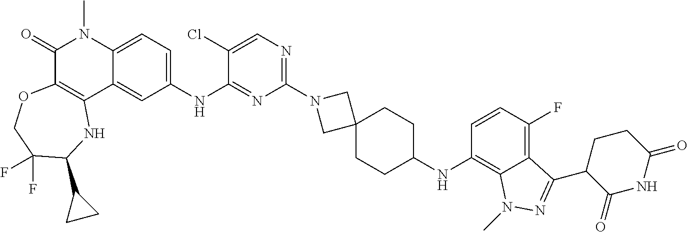 [SMILES: Cn1nc(C2CCC(=O)NC2=O)c2c(F)ccc(NC3CCC4(CC3)CN(c3ncc(Cl)c(Nc5ccc6c(c5)c5c(c(=O)n6C)OCC(F)(F)[C@H](C6CC6)N5)n3)C4)c21] |
| 503 | 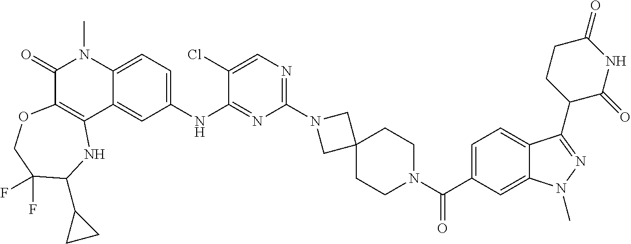 [SMILES: Cn1nc(C2CCC(=O)NC2=O)c2ccc(C(=O)N3CCC4(CC3)CN(c3ncc(Cl)c(Nc5ccc6c(c5)c5c(c(=O)n6C)OCC(F)(F)C(C6CC6)N5)n3)C4)cc21] |
| 503a | 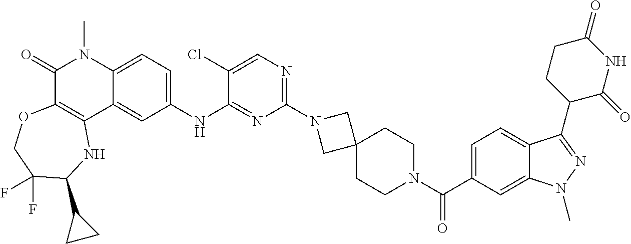 [SMILES: Cn1nc(C2CCC(=O)NC2=O)c2ccc(C(=O)N3CCC4(CC3)CN(c3ncc(Cl)c(Nc5ccc6c(c5)c5c(c(=O)n6C)OCC(F)(F)[C@H](C6CC6)N5)n3)C4)cc21] |
| 504 | 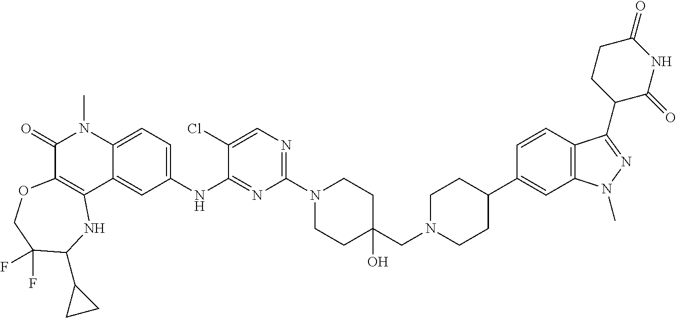 [SMILES: Cn1nc(C2CCC(=O)NC2=O)c2ccc(C3CCN(CC4(O)CCN(c5ncc(Cl)c(Nc6ccc7c(c6)c6c(c(=O)n7C)OCC(F)(F)C(C7CC7)N6)n5)CC4)CC3)cc21] |
| 504a | 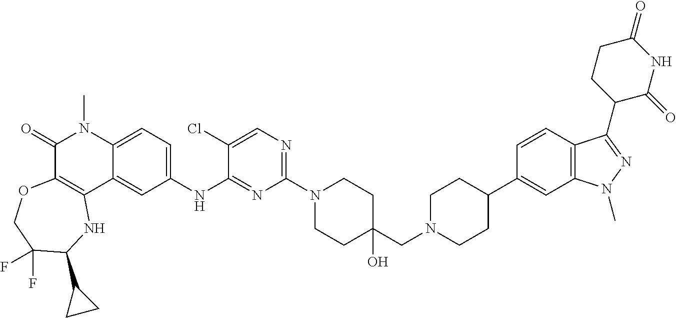 [SMILES: Cn1nc(C2CCC(=O)NC2=O)c2ccc(C3CCN(CC4(O)CCN(c5ncc(Cl)c(Nc6ccc7c(c6)c6c(c(=O)n7C)OCC(F)(F)[C@H](C7CC7)N6)n5)CC4)CC3)cc21] |
| 505 | 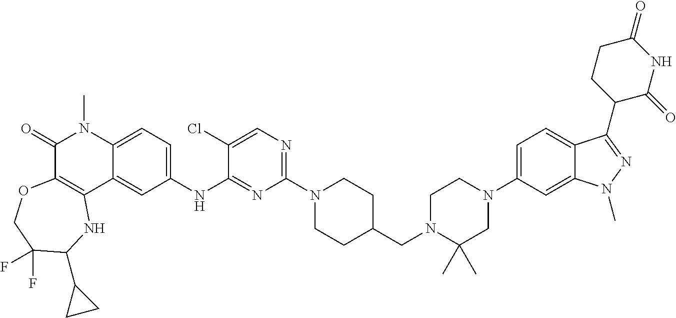 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)CC4)C(C)(C)C3)cc21] |
| 505a | 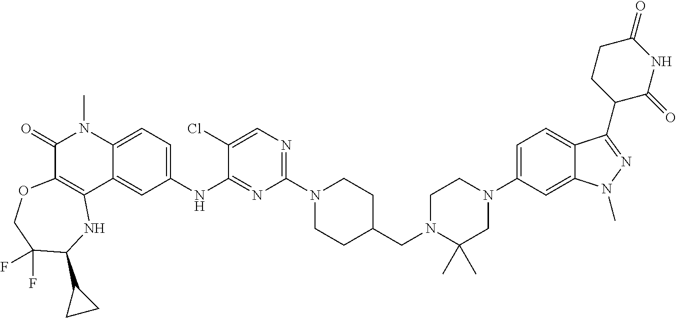 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)C(C)(C)C3)cc21] |
| 506 | 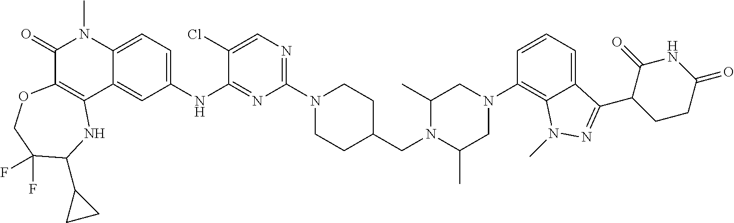 [SMILES: CC1CN(c2cccc3c(C4CCC(=O)NC4=O)nn(C)c23)CC(C)N1CC1CCN(c2ncc(Cl)c(Nc3ccc4c(c3)c3c(c(=O)n4C)OCC(F)(F)C(C4CC4)N3)n2)CC1] |
| 506a | 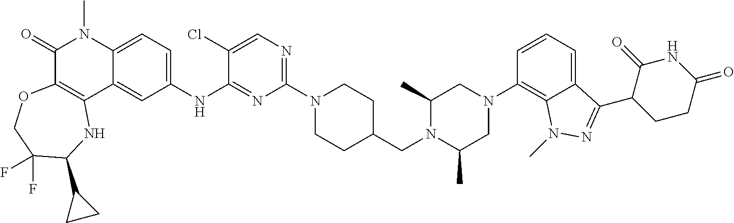 [SMILES: C[C@@H]1CN(c2cccc3c(C4CCC(=O)NC4=O)nn(C)c23)C[C@H](C)N1CC1CCN(c2ncc(Cl)c(Nc3ccc4c(c3)c3c(c(=O)n4C)OCC(F)(F)[C@H](C4CC4)N3)n2)CC1] |
| 506b | 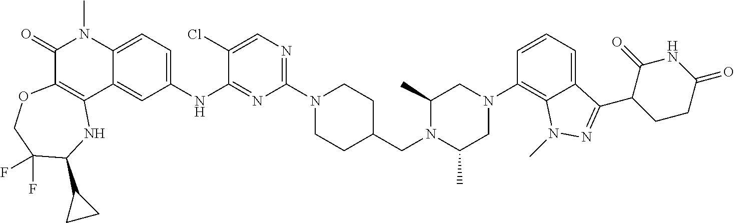 [SMILES: C[C@H]1CN(c2cccc3c(C4CCC(=O)NC4=O)nn(C)c23)C[C@H](C)N1CC1CCN(c2ncc(Cl)c(Nc3ccc4c(c3)c3c(c(=O)n4C)OCC(F)(F)[C@H](C4CC4)N3)n2)CC1] |
| 507 | 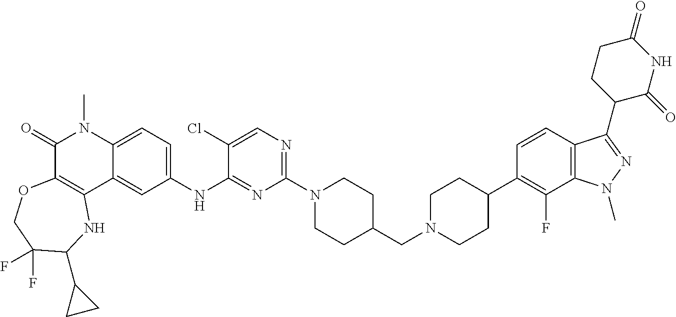 [SMILES: Cn1nc(C2CCC(=O)NC2=O)c2ccc(C3CCN(CC4CCN(c5ncc(Cl)c(Nc6ccc7c(c6)c6c(c(=O)n7C)OCC(F)(F)C(C7CC7)N6)n5)CC4)CC3)c(F)c21] |
| 507a | 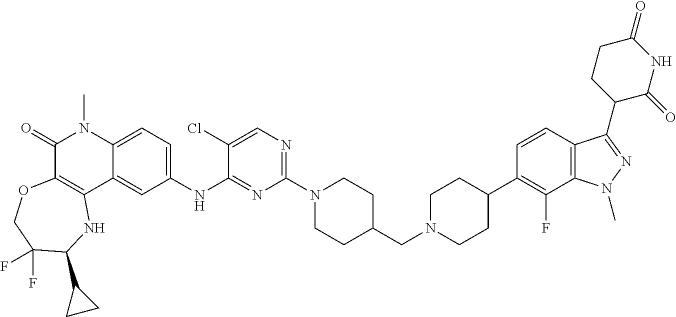 [SMILES: Cn1nc(C2CCC(=O)NC2=O)c2ccc(C3CCN(CC4CCN(c5ncc(Cl)c(Nc6ccc7c(c6)c6c(c(=O)n7C)OCC(F)(F)[C@H](C7CC7)N6)n5)CC4)CC3)c(F)c21] |
| 508 | 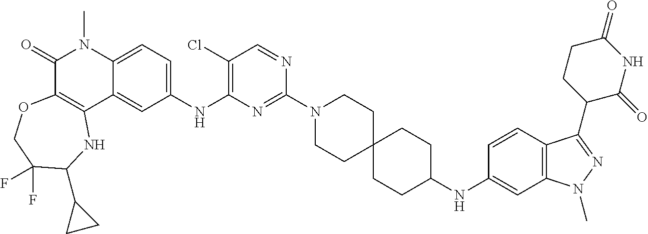 [SMILES: Cn1nc(C2CCC(=O)NC2=O)c2ccc(NC3CCC4(CC3)CCN(c3ncc(Cl)c(Nc5ccc6c(c5)c5c(c(=O)n6C)OCC(F)(F)C(C6CC6)N5)n3)CC4)cc21] |
| 508a | 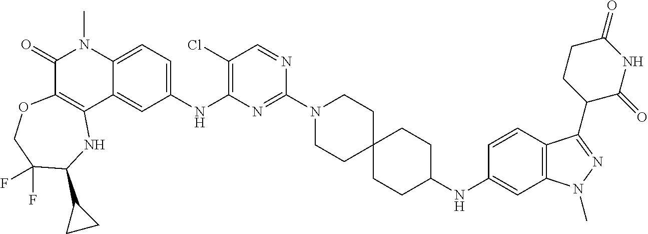 [SMILES: Cn1nc(C2CCC(=O)NC2=O)c2ccc(NC3CCC4(CC3)CCN(c3ncc(Cl)c(Nc5ccc6c(c5)c5c(c(=O)n6C)OCC(F)(F)[C@H](C6CC6)N5)n3)CC4)cc21] |
| 509 | 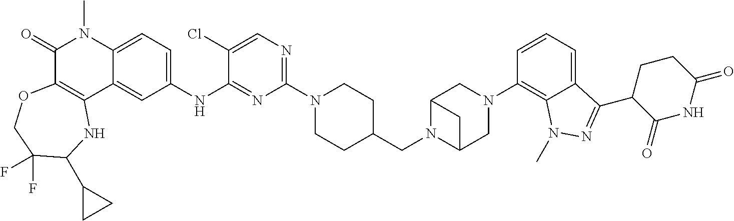 [SMILES: Cn1nc(C2CCC(=O)NC2=O)c2cccc(N3CC4CC(C3)N4CC3CCN(c4ncc(Cl)c(Nc5ccc6c(c5)c5c(c(=O)n6C)OCC(F)(F)C(C6CC6)N5)n4)CC3)c21] |
| 509a | 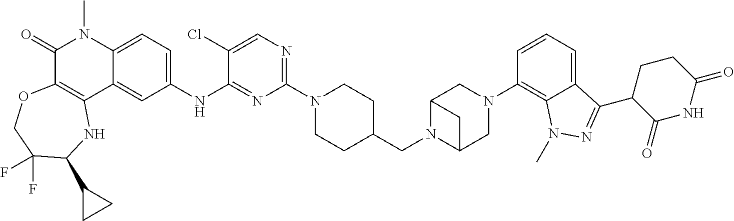 [SMILES: Cn1nc(C2CCC(=O)NC2=O)c2cccc(N3CC4CC(C3)N4CC3CCN(c4ncc(Cl)c(Nc5ccc6c(c5)c5c(c(=O)n6C)OCC(F)(F)[C@H](C6CC6)N5)n4)CC3)c21] |
| 510 | 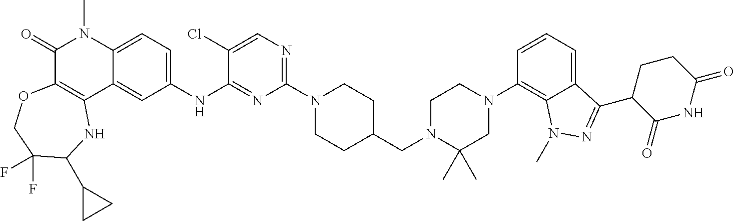 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)C(C7CC7)N6)n5)CC4)C(C)(C)C3)c21] |
| 510a | 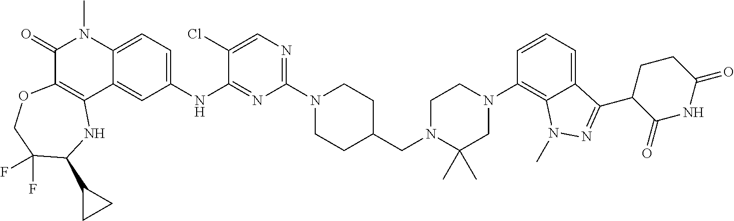 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)[C@H](C7CC7)N6)n5)CC4)C(C)(C)C3)c21] |
| 511 | 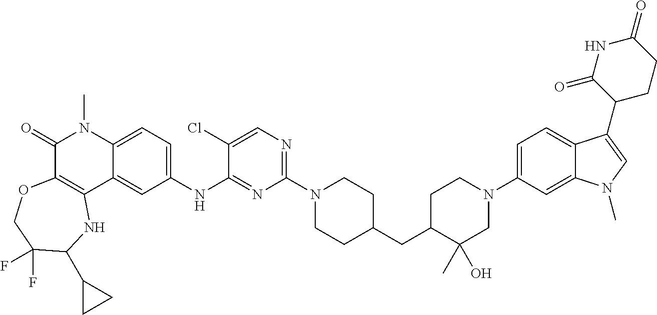 [SMILES: Cn1cc(C2CCC(=O)NC2=O)c2ccc(N3CCC(CC4CCN(c5ncc(Cl)c(Nc6ccc7c(c6)c6c(c(=O)n7C)OCC(F)(F)C(C7CC7)N6)n5)CC4)C(C)(O)C3)cc21] |
| 511a | 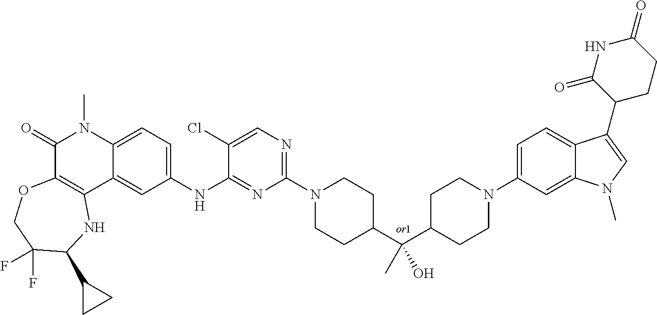 [SMILES: Cn1cc(C2CCC(=O)NC2=O)c2ccc(N3CCC([C@](C)(O)C4CCN(c5ncc(Cl)c(Nc6ccc7c(c6)c6c(c(=O)n7C)OCC(F)(F)[C@H](C7CC7)N6)n5)CC4)CC3)cc21] |
| 511b | 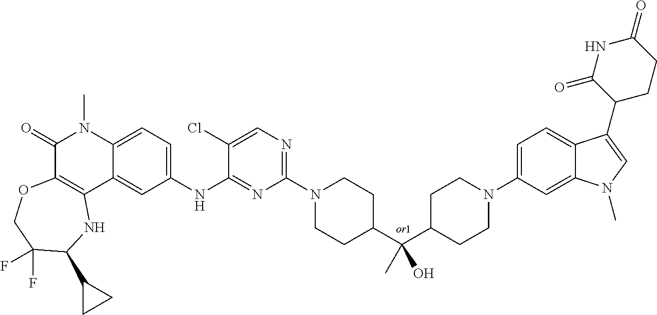 [SMILES: Cn1cc(C2CCC(=O)NC2=O)c2ccc(N3CCC([C@@](C)(O)C4CCN(c5ncc(Cl)c(Nc6ccc7c(c6)c6c(c(=O)n7C)OCC(F)(F)[C@H](C7CC7)N6)n5)CC4)CC3)cc21] |
| 512 | 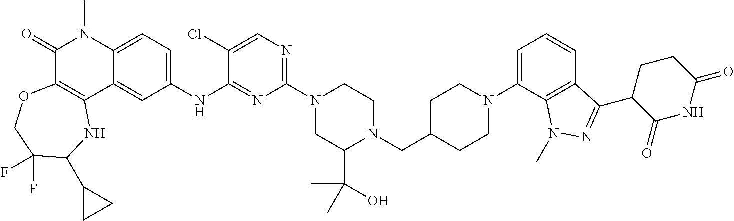 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)C(C7CC7)N6)n5)CC4C(C)(C)O)CC3)c21] |
| 512a | 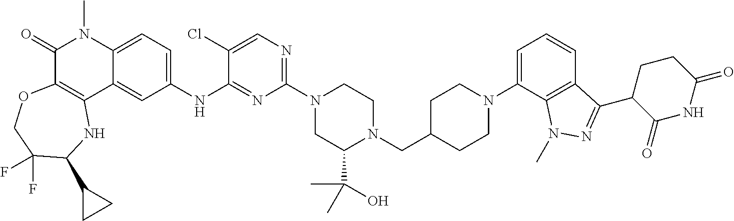 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@H]4C(C)(C)O)CC3)c21] |
| 512b | 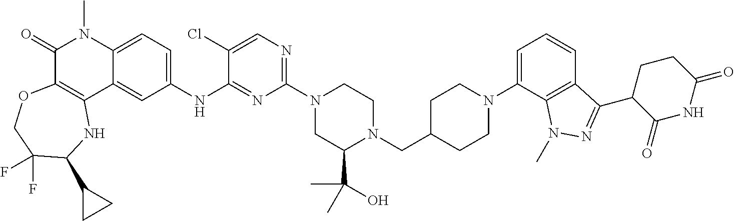 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@@H]4C(C)(C)O)CC3)c21] |
| 513 | 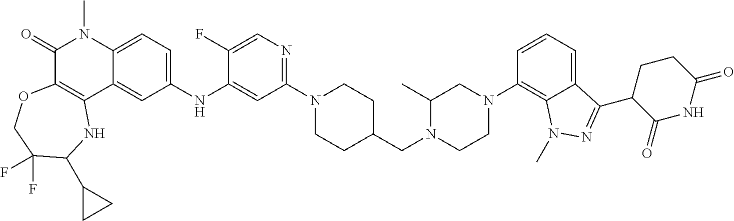 [SMILES: CC1CN(c2cccc3c(C4CCC(=O)NC4=O)nn(C)c23)CCN1CC1CCN(c2cc(Nc3ccc4c(c3)c3c(c(=O)n4C)OCC(F)(F)C(C4CC4)N3)c(F)cn2)CC1] |
| 513a | 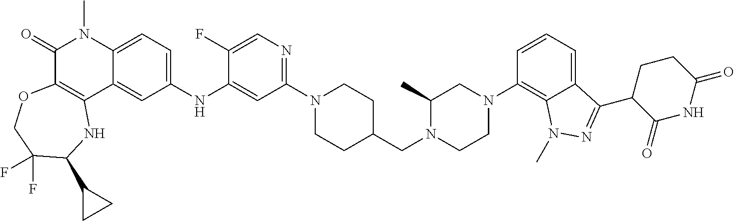 [SMILES: C[C@H]1CN(c2cccc3c(C4CCC(=O)NC4=O)nn(C)c23)CCN1CC1CCN(c2cc(Nc3ccc4c(c3)c3c(c(=O)n4C)OCC(F)(F)[C@H](C4CC4)N3)c(F)cn2)CC1] |
| 514 | 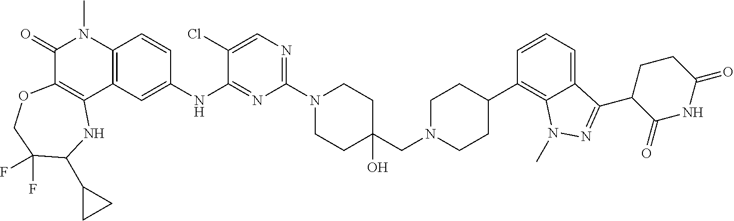 [SMILES: Cn1nc(C2CCC(=O)NC2=O)c2cccc(C3CCN(CC4(O)CCN(c5ncc(Cl)c(Nc6ccc7c(c6)c6c(c(=O)n7C)OCC(F)(F)C(C7CC7)N6)n5)CC4)CC3)c21] |
| 514a | 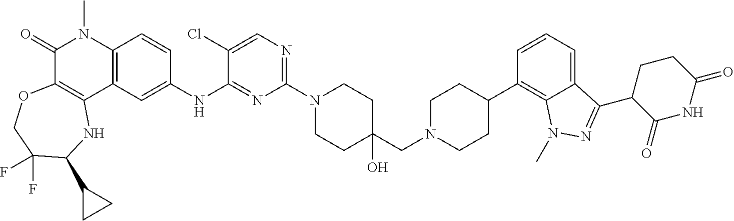 [SMILES: Cn1nc(C2CCC(=O)NC2=O)c2cccc(C3CCN(CC4(O)CCN(c5ncc(Cl)c(Nc6ccc7c(c6)c6c(c(=O)n7C)OCC(F)(F)[C@H](C7CC7)N6)n5)CC4)CC3)c21] |
| 516 | 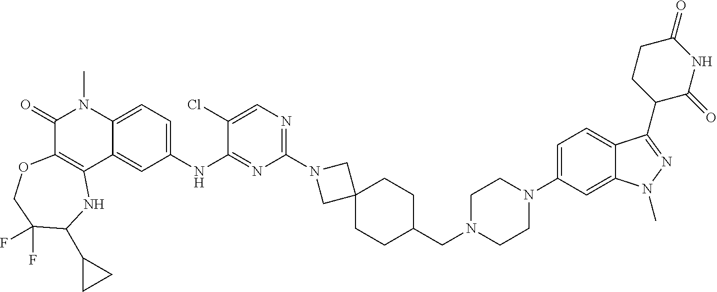 [SMILES: Cn1nc(C2CCC(=O)NC2=O)c2ccc(N3CCN(CC4CCC5(CC4)CN(c4ncc(Cl)c(Nc6ccc7c(c6)c6c(c(=O)n7C)OCC(F)(F)C(C7CC7)N6)n4)C5)CC3)cc21] |
| 516a | 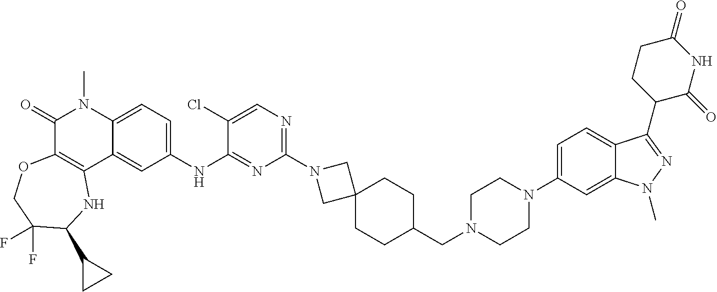 [SMILES: Cn1nc(C2CCC(=O)NC2=O)c2ccc(N3CCN(CC4CCC5(CC4)CN(c4ncc(Cl)c(Nc6ccc7c(c6)c6c(c(=O)n7C)OCC(F)(F)[C@H](C7CC7)N6)n4)C5)CC3)cc21] |
| 518 | 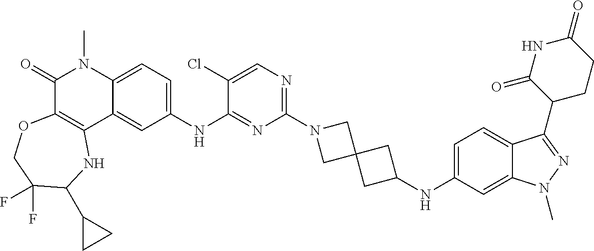 [SMILES: Cn1nc(C2CCC(=O)NC2=O)c2ccc(NC3CC4(C3)CN(c3ncc(Cl)c(Nc5ccc6c(c5)c5c(c(=O)n6C)OCC(F)(F)C(C6CC6)N5)n3)C4)cc21] |
| 518a | 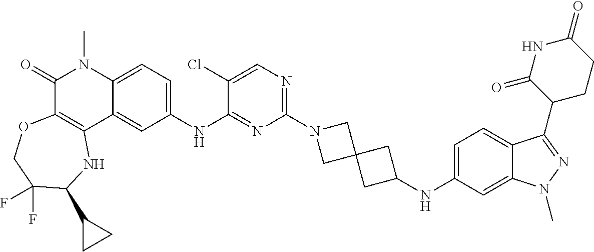 [SMILES: Cn1nc(C2CCC(=O)NC2=O)c2ccc(NC3CC4(C3)CN(c3ncc(Cl)c(Nc5ccc6c(c5)c5c(c(=O)n6C)OCC(F)(F)[C@H](C6CC6)N5)n3)C4)cc21] |
| 519 | 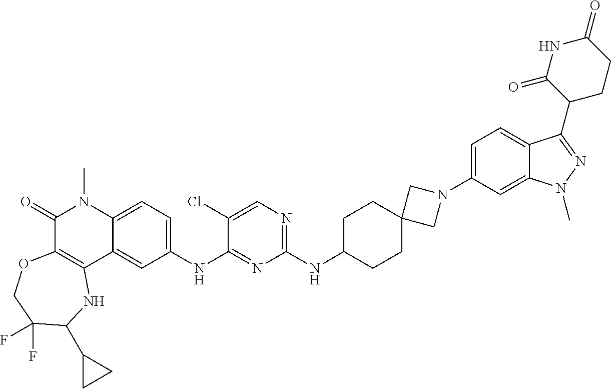 [SMILES: Cn1nc(C2CCC(=O)NC2=O)c2ccc(N3CC4(CCC(Nc5ncc(Cl)c(Nc6ccc7c(c6)c6c(c(=O)n7C)OCC(F)(F)C(C7CC7)N6)n5)CC4)C3)cc21] |
| 519a | 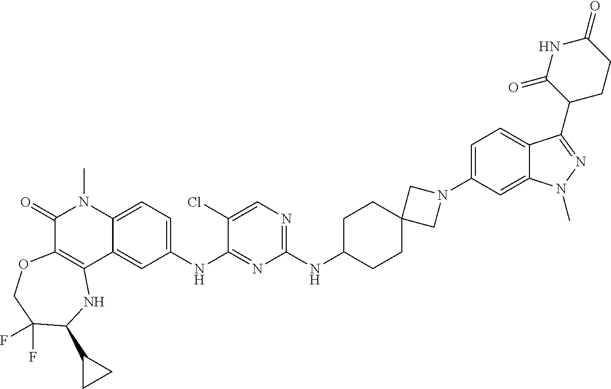 [SMILES: Cn1nc(C2CCC(=O)NC2=O)c2ccc(N3CC4(CCC(Nc5ncc(Cl)c(Nc6ccc7c(c6)c6c(c(=O)n7C)OCC(F)(F)[C@H](C7CC7)N6)n5)CC4)C3)cc21] |
| 520 | 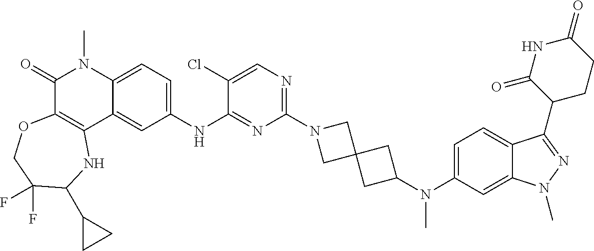 [SMILES: CN(c1ccc2c(C3CCC(=O)NC3=O)nn(C)c2c1)C1CC2(C1)CN(c1ncc(Cl)c(Nc3ccc4c(c3)c3c(c(=O)n4C)OCC(F)(F)C(C4CC4)N3)n1)C2] |
| 520a | 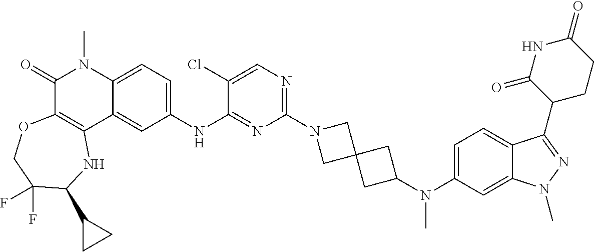 [SMILES: CN(c1ccc2c(C3CCC(=O)NC3=O)nn(C)c2c1)C1CC2(C1)CN(c1ncc(Cl)c(Nc3ccc4c(c3)c3c(c(=O)n4C)OCC(F)(F)[C@H](C4CC4)N3)n1)C2] |
| 521 | 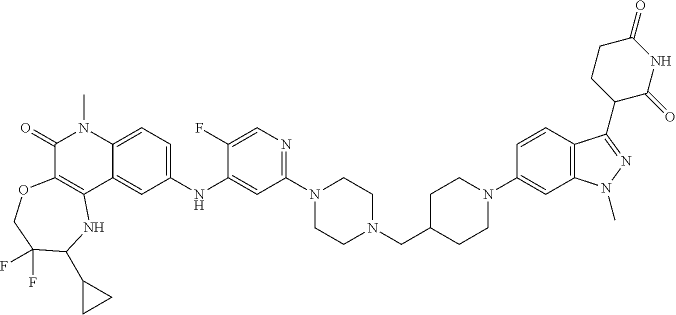 [SMILES: Cn1nc(C2CCC(=O)NC2=O)c2ccc(N3CCC(CN4CCN(c5cc(Nc6ccc7c(c6)c6c(c(=O)n7C)OCC(F)(F)C(C7CC7)N6)c(F)cn5)CC4)CC3)cc21] |
| 521a | 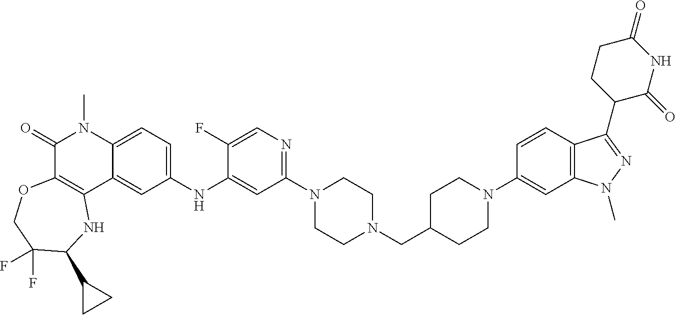 [SMILES: Cn1nc(C2CCC(=O)NC2=O)c2ccc(N3CCC(CN4CCN(c5cc(Nc6ccc7c(c6)c6c(c(=O)n7C)OCC(F)(F)[C@H](C7CC7)N6)c(F)cn5)CC4)CC3)cc21] |
| 522 | 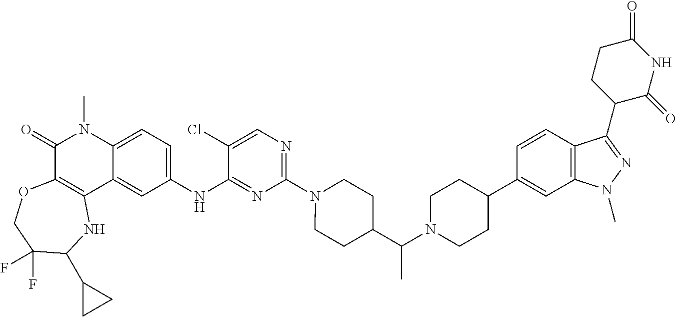 [SMILES: CC(C1CCN(c2ncc(Cl)c(Nc3ccc4c(c3)c3c(c(=O)n4C)OCC(F)(F)C(C4CC4)N3)n2)CC1)N1CCC(c2ccc3c(C4CCC(=O)NC4=O)nn(C)c3c2)CC1] |
| 522a | 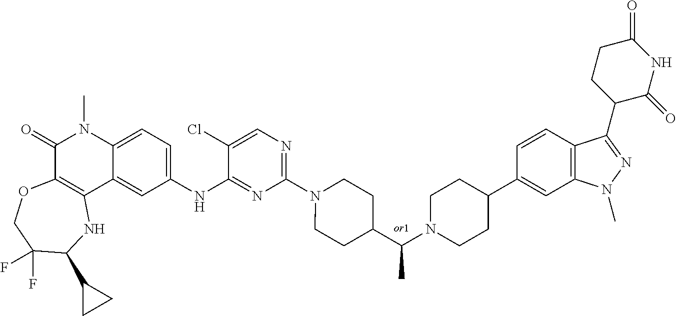 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCC(c2ccc3c(C4CCC(=O)NC4=O)nn(C)c3c2)CC1] |
| 522b | 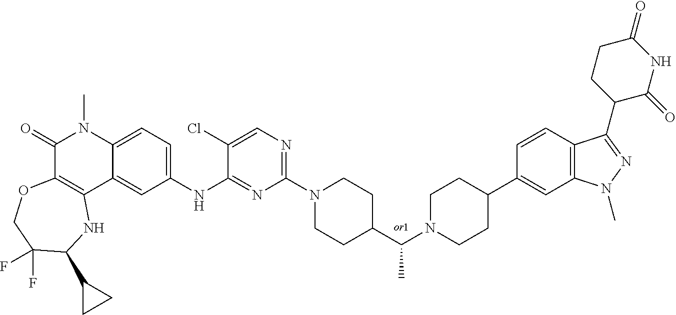 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCC(c2ccc3c(C4CCC(=O)NC4=O)nn(C)c3c2)CC1] |
| 523 | 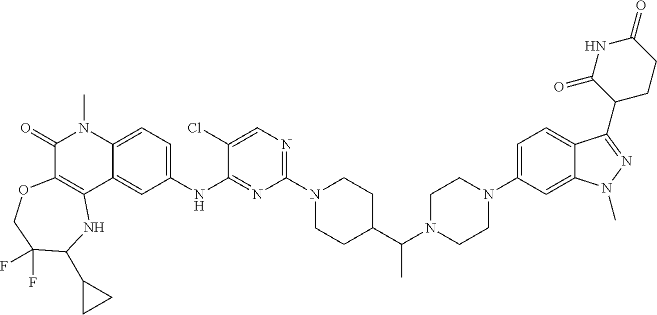 [SMILES: CC(C1CCN(c2ncc(Cl)c(Nc3ccc4c(c3)c3c(c(=O)n4C)OCC(F)(F)C(C4CC4)N3)n2)CC1)N1CCN(c2ccc3c(C4CCC(=O)NC4=O)nn(C)c3c2)CC1] |
| 523a | 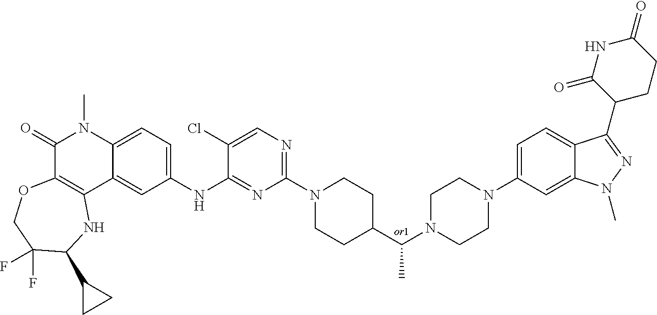 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCN(c2ccc3c(C4CCC(=O)NC4=O)nn(C)c3c2)CC1] |
| 523b | 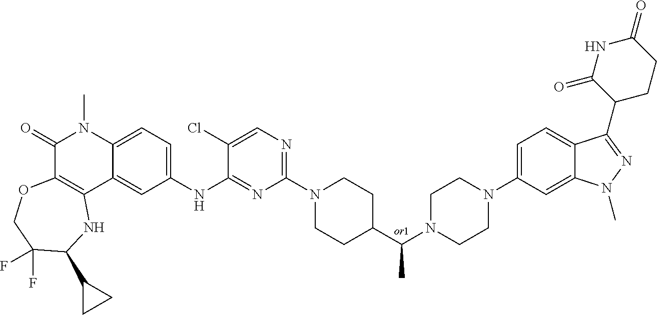 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCN(c2ccc3c(C4CCC(=O)NC4=O)nn(C)c3c2)CC1] |
| 524 | 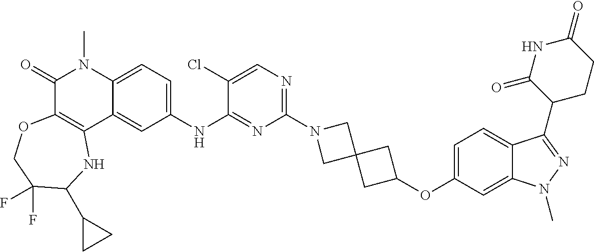 [SMILES: Cn1nc(C2CCC(=O)NC2=O)c2ccc(OC3CC4(C3)CN(c3ncc(Cl)c(Nc5ccc6c(c5)c5c(c(=O)n6C)OCC(F)(F)C(C6CC6)N5)n3)C4)cc21] |
| 524a | 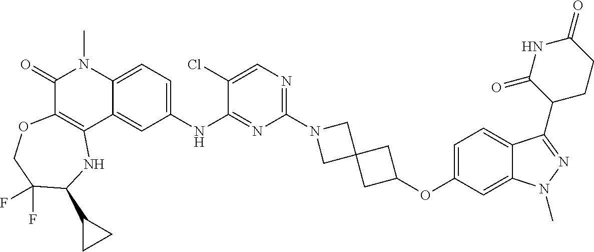 [SMILES: Cn1nc(C2CCC(=O)NC2=O)c2ccc(OC3CC4(C3)CN(c3ncc(Cl)c(Nc5ccc6c(c5)c5c(c(=O)n6C)OCC(F)(F)[C@H](C6CC6)N5)n3)C4)cc21] |
| 525 | 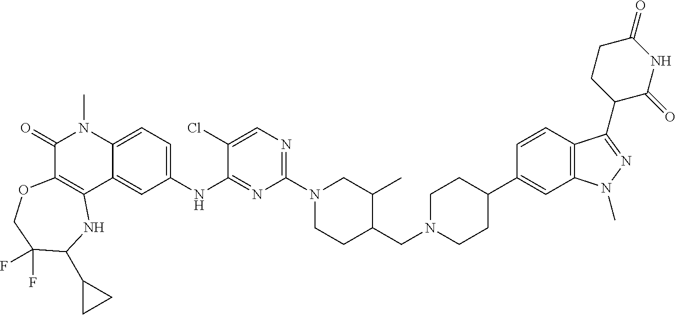 [SMILES: CC1CN(c2ncc(Cl)c(Nc3ccc4c(c3)c3c(c(=O)n4C)OCC(F)(F)C(C4CC4)N3)n2)CCC1CN1CCC(c2ccc3c(C4CCC(=O)NC4=O)nn(C)c3c2)CC1] |
| 525a | 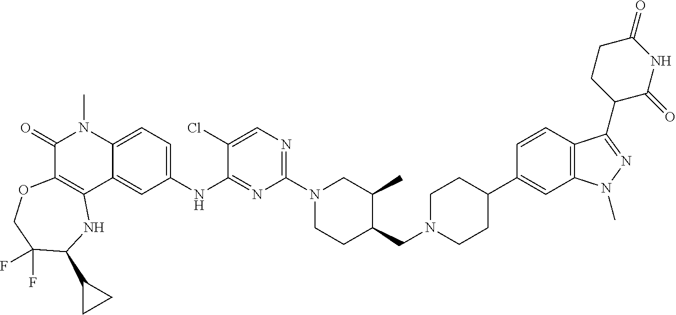 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCC(c2ccc3c(C4CCC(=O)NC4=O)nn(C)c3c2)CC1] |
| 525b | 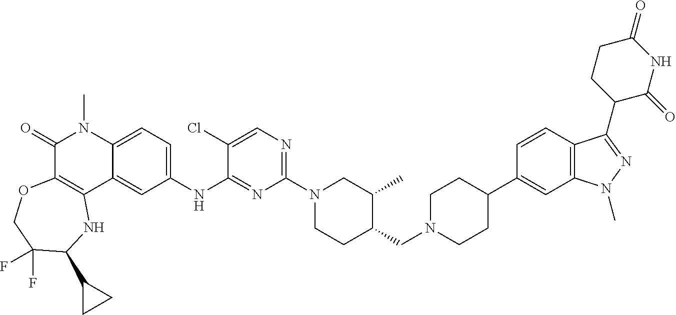 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCC(c2ccc3c(C4CCC(=O)NC4=O)nn(C)c3c2)CC1] |
| 525c | 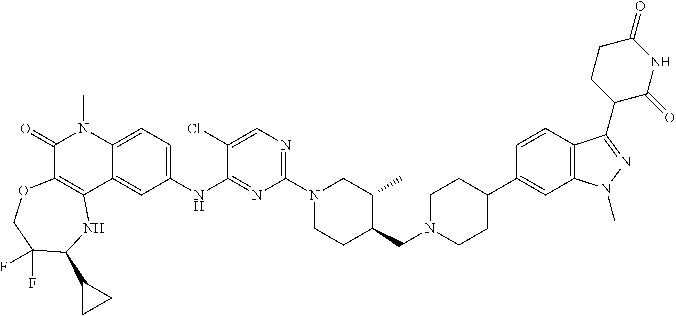 [SMILES: C[C@H]1CN(c2ncc(Cl)c(Nc3ccc4c(c3)c3c(c(=O)n4C)OCC(F)(F)[C@H](C4CC4)N3)n2)CC[C@@H]1CN1CCC(c2ccc3c(C4CCC(=O)NC4=O)nn(C)c3c2)CC1] |
| 525d | 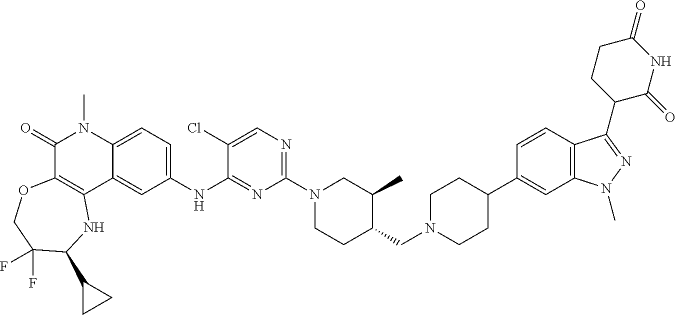 [SMILES: C[C@@H]1CN(c2ncc(Cl)c(Nc3ccc4c(c3)c3c(c(=O)n4C)OCC(F)(F)[C@H](C4CC4)N3)n2)CC[C@H]1CN1CCC(c2ccc3c(C4CCC(=O)NC4=O)nn(C)c3c2)CC1] |
| 526 | 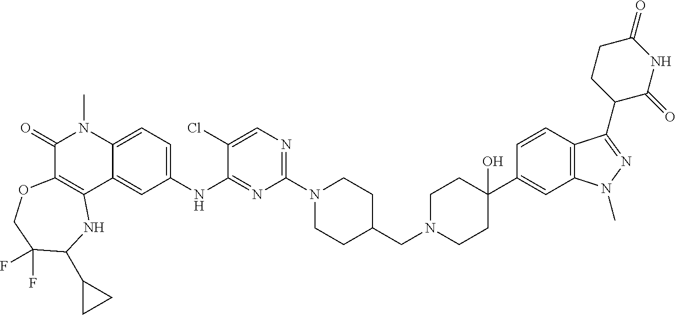 [SMILES: Cn1nc(C2CCC(=O)NC2=O)c2ccc(C3(O)CCN(CC4CCN(c5ncc(Cl)c(Nc6ccc7c(c6)c6c(c(=O)n7C)OCC(F)(F)C(C7CC7)N6)n5)CC4)CC3)cc21] |
| 526a | 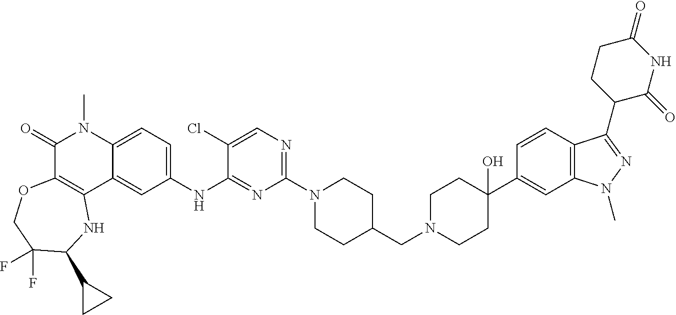 [SMILES: Cn1nc(C2CCC(=O)NC2=O)c2ccc(C3(O)CCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 527 | 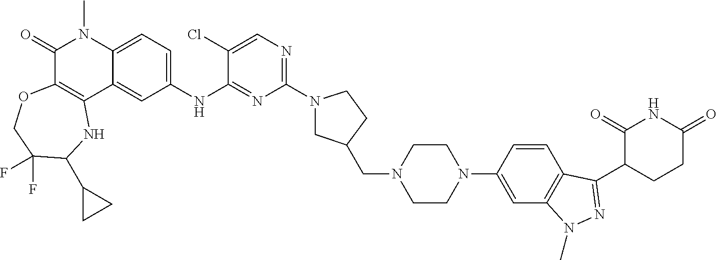 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)C4)CC3)cc21] |
| 527a | 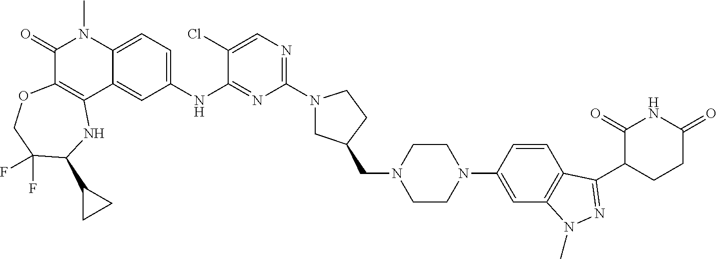 [SMILES: Cn1nc(C2CCC(=O)NC2=O)c2ccc(N3CCN(C[C@@H]4CCN(c5ncc(Cl)c(Nc6ccc7c(c6)c6c(c(=O)n7C)OCC(F)(F)[C@H](C7CC7)N6)n5)C4)CC3)cc21] |
| 527b | 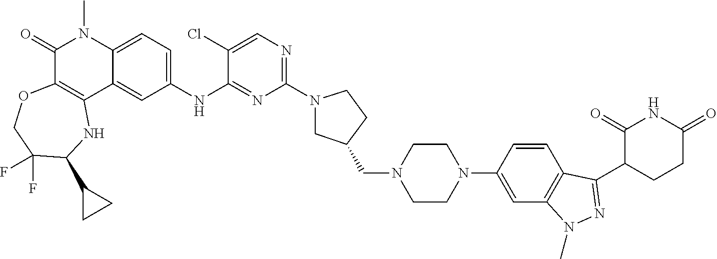 [SMILES: Cn1nc(C2CCC(=O)NC2=O)c2ccc(N3CCN(C[C@H]4CCN(c5ncc(Cl)c(Nc6ccc7c(c6)c6c(c(=O)n7C)OCC(F)(F)[C@H](C7CC7)N6)n5)C4)CC3)cc21] |
| 528 | 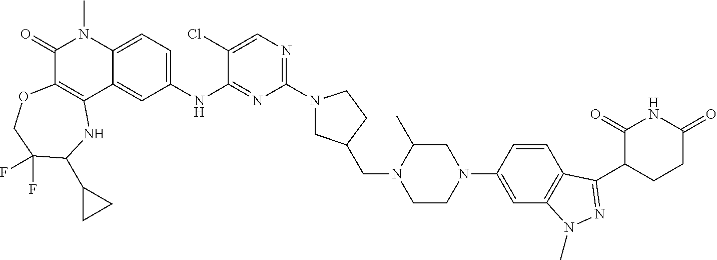 [SMILES: CC1CN(c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)C(C4CC4)N3)n2)C1] |
| 528a | 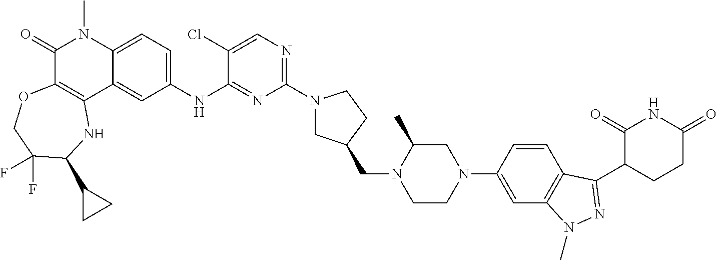 [SMILES: C[C@H]1CN(c2ccc3c(C4CCC(=O)NC4=O)nn(C)c3c2)CCN1C[C@@H]1CCN(c2ncc(Cl)c(Nc3ccc4c(c3)c3c(c(=O)n4C)OCC(F)(F)[C@H](C4CC4)N3)n2)C1] |
| 529 | 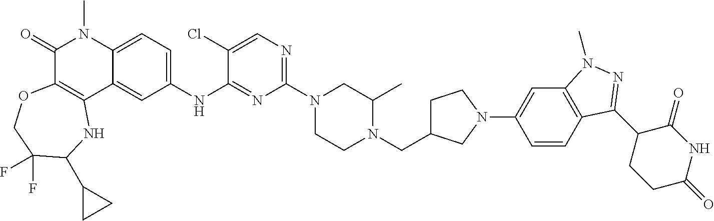 [SMILES: CC1CN(c2ncc(Cl)c(Nc3ccc4c(c3)c3c(c(=O)n4C)OCC(F)(F)C(C4CC4)N3)n2)CCN1CC1CCN(c2ccc3c(C4CCC(=O)NC4=O)nn(C)c3c2)C1] |
| 529a | 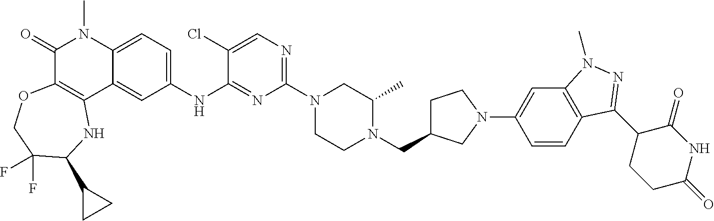 [SMILES: C[C@H]1CN(c2ncc(Cl)c(Nc3ccc4c(c3)c3c(c(=O)n4C)OCC(F)(F)[C@H](C4CC4)N3)n2)CCN1C[C@H]1CCN(c2ccc3c(C4CCC(=O)NC4=O)nn(C)c3c2)C1] |
| 529b | 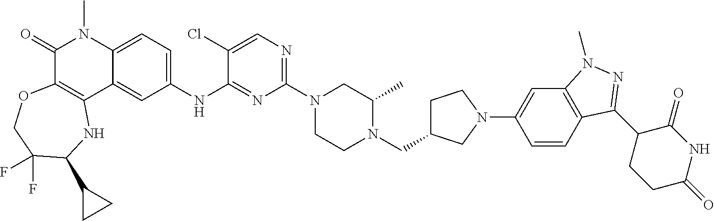 [SMILES: C[C@H]1CN(c2ncc(Cl)c(Nc3ccc4c(c3)c3c(c(=O)n4C)OCC(F)(F)[C@H](C4CC4)N3)n2)CCN1C[C@@H]1CCN(c2ccc3c(C4CCC(=O)NC4=O)nn(C)c3c2)C1] |
| 529c | 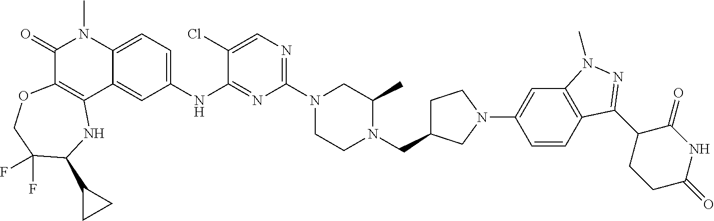 [SMILES: C[C@@H]1CN(c2ncc(Cl)c(Nc3ccc4c(c3)c3c(c(=O)n4C)OCC(F)(F)[C@H](C4CC4)N3)n2)CCN1C[C@H]1CCN(c2ccc3c(C4CCC(=O)NC4=O)nn(C)c3c2)C1] |
| 530 | 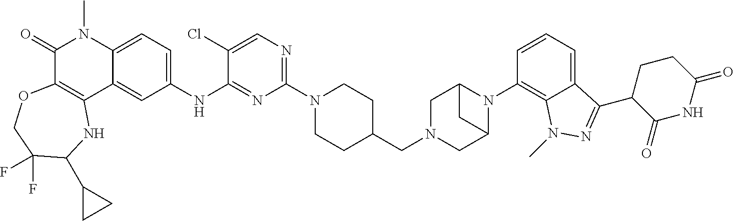 [SMILES: Cn1nc(C2CCC(=O)NC2=O)c2cccc(N3C4CC3CN(CC3CCN(c5ncc(Cl)c(Nc6ccc7c(c6)c6c(c(=O)n7C)OCC(F)(F)C(C7CC7)N6)n5)CC3)C4)c21] |
| 530a | 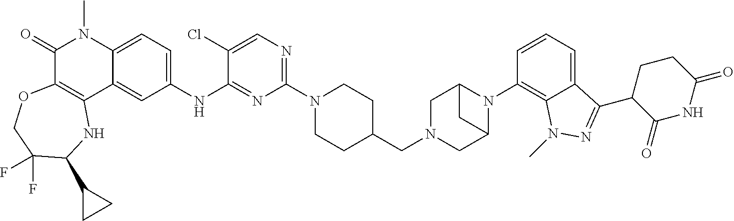 [SMILES: Cn1nc(C2CCC(=O)NC2=O)c2cccc(N3C4CC3CN(CC3CCN(c5ncc(Cl)c(Nc6ccc7c(c6)c6c(c(=O)n7C)OCC(F)(F)[C@H](C7CC7)N6)n5)CC3)C4)c21] |
| 531 | 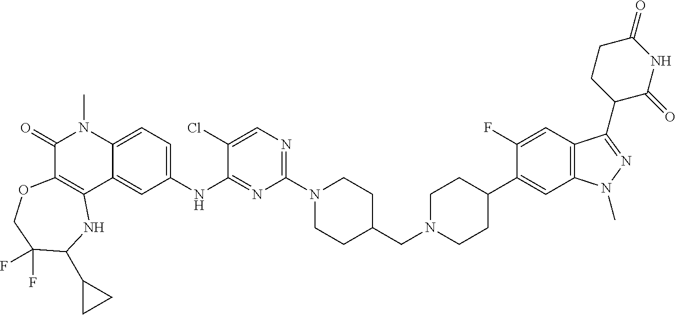 [SMILES: Cn1nc(C2CCC(=O)NC2=O)c2cc(F)c(C3CCN(CC4CCN(c5ncc(Cl)c(Nc6ccc7c(c6)c6c(c(=O)n7C)OCC(F)(F)C(C7CC7)N6)n5)CC4)CC3)cc21] |
| 531a | 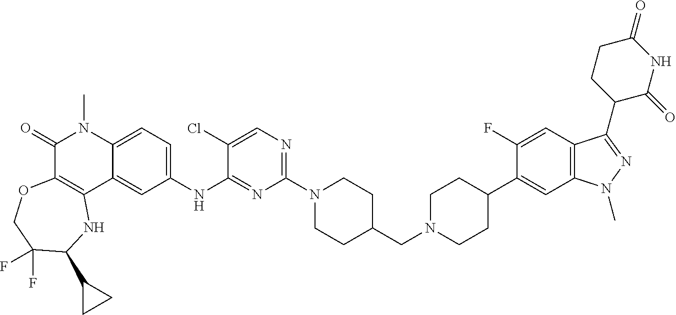 [SMILES: Cn1nc(C2CCC(=O)NC2=O)c2cc(F)c(C3CCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 532 | 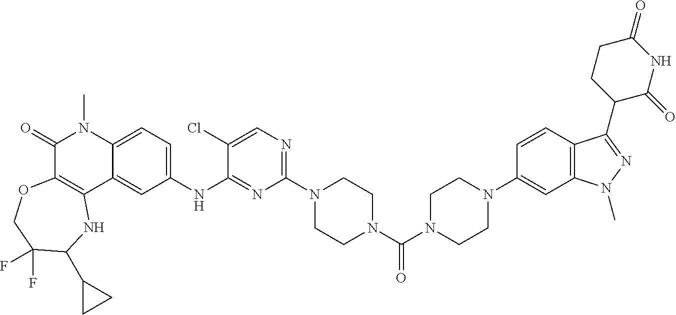 [SMILES: Cn1nc(C2CCC(=O)NC2=O)c2ccc(N3CCN(C(=O)N4CCN(c5ncc(Cl)c(Nc6ccc7c(c6)c6c(c(=O)n7C)OCC(F)(F)C(C7CC7)N6)n5)CC4)CC3)cc21] |
| 532a | 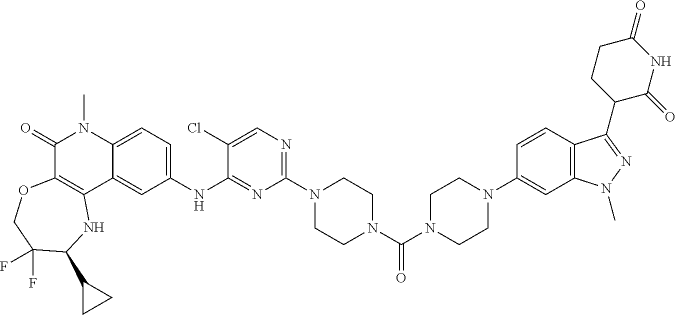 [SMILES: Cn1nc(C2CCC(=O)NC2=O)c2ccc(N3CCN(C(=O)N4CCN(c5ncc(Cl)c(Nc6ccc7c(c6)c6c(c(=O)n7C)OCC(F)(F)[C@H](C7CC7)N6)n5)CC4)CC3)cc21] |
| 533 | 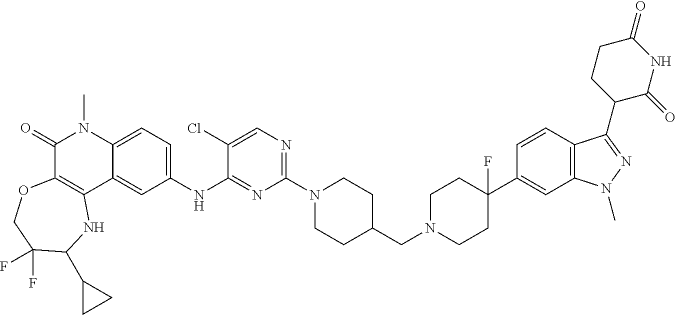 [SMILES: Cn1nc(C2CCC(=O)NC2=O)c2ccc(C3(F)CCN(CC4CCN(c5ncc(Cl)c(Nc6ccc7c(c6)c6c(c(=O)n7C)OCC(F)(F)C(C7CC7)N6)n5)CC4)CC3)cc21] |
| 533a | 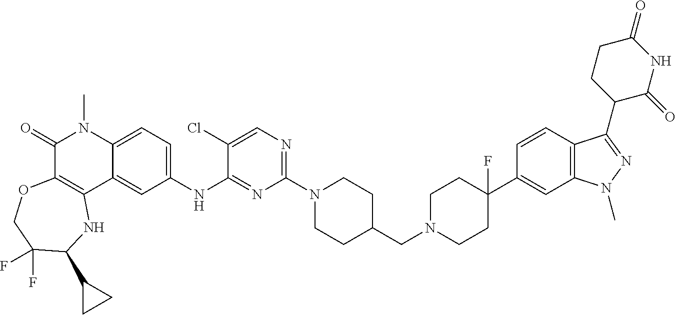 [SMILES: Cn1nc(C2CCC(=O)NC2=O)c2ccc(C3(F)CCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 534 | 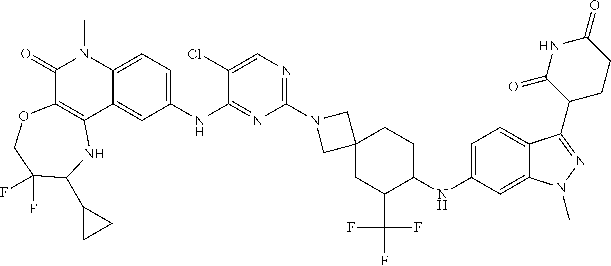 [SMILES: Cn1nc(C2CCC(=O)NC2=O)c2ccc(NC3CCC4(CC3C(F)(F)F)CN(c3ncc(Cl)c(Nc5ccc6c(c5)c5c(c(=O)n6C)OCC(F)(F)C(C6CC6)N5)n3)C4)cc21] |
| 534a | 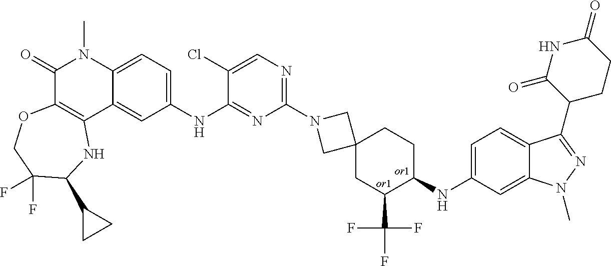 [SMILES: Cn1nc(C2CCC(=O)NC2=O)c2ccc(N[C@@H]3CCC4(C[C@@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| 534b | 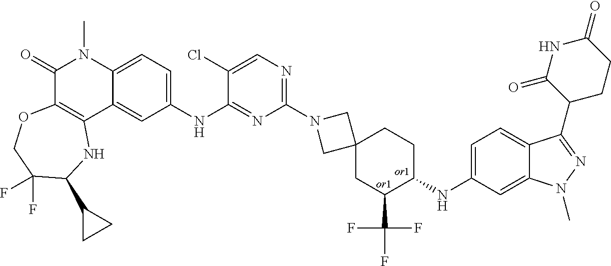 [SMILES: Cn1nc(C2CCC(=O)NC2=O)c2ccc(N[C@H]3CCC4(C[C@@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| 534c | 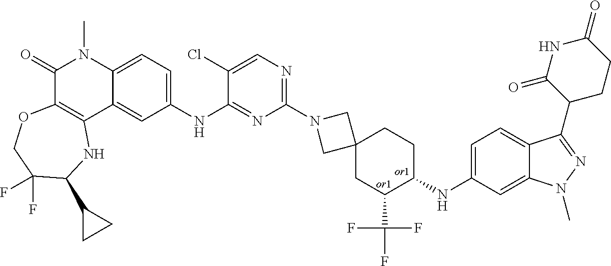 [SMILES: Cn1nc(C2CCC(=O)NC2=O)c2ccc(N[C@H]3CCC4(C[C@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| 534d | 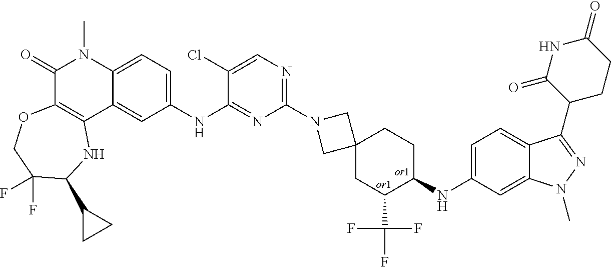 [SMILES: Cn1nc(C2CCC(=O)NC2=O)c2ccc(N[C@@H]3CCC4(C[C@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| 535 | 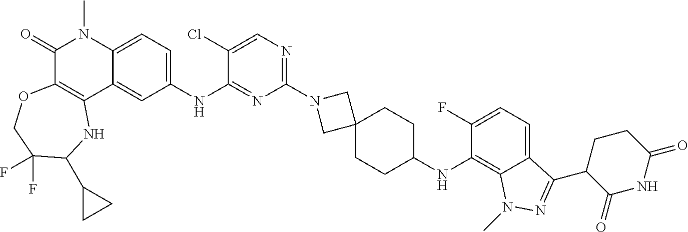 [SMILES: Cn1nc(C2CCC(=O)NC2=O)c2ccc(F)c(NC3CCC4(CC3)CN(c3ncc(Cl)c(Nc5ccc6c(c5)c5c(c(=O)n6C)OCC(F)(F)C(C6CC6)N5)n3)C4)c21] |
| 535a | 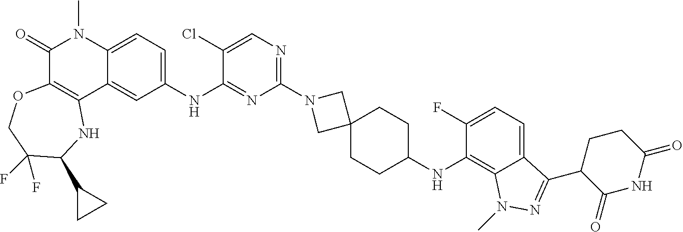 [SMILES: Cn1nc(C2CCC(=O)NC2=O)c2ccc(F)c(NC3CCC4(CC3)CN(c3ncc(Cl)c(Nc5ccc6c(c5)c5c(c(=O)n6C)OCC(F)(F)[C@H](C6CC6)N5)n3)C4)c21] |
| 536 | 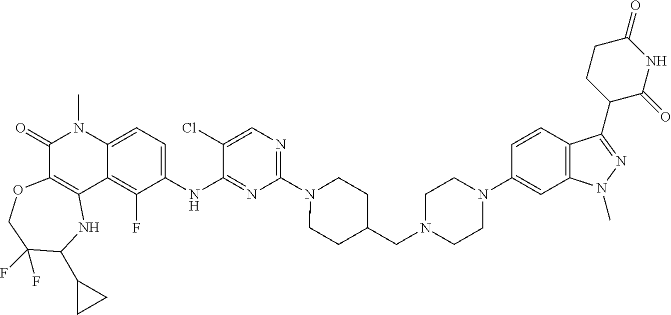 [SMILES: Cn1nc(C2CCC(=O)NC2=O)c2ccc(N3CCN(CC4CCN(c5ncc(Cl)c(Nc6ccc7c(c6F)c6c(c(=O)n7C)OCC(F)(F)C(C7CC7)N6)n5)CC4)CC3)cc21] |
| 536a | 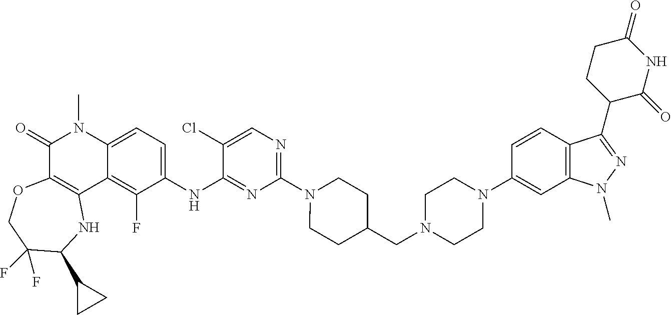 [SMILES: Cn1nc(C2CCC(=O)NC2=O)c2ccc(N3CCN(CC4CCN(c5ncc(Cl)c(Nc6ccc7c(c6F)c6c(c(=O)n7C)OCC(F)(F)[C@H](C7CC7)N6)n5)CC4)CC3)cc21] |
| 537 | 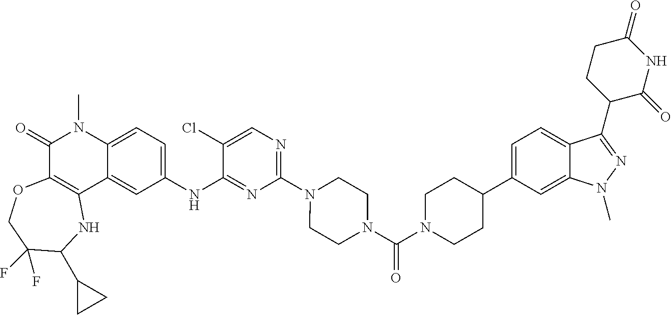 [SMILES: Cn1nc(C2CCC(=O)NC2=O)c2ccc(C3CCN(C(=O)N4CCN(c5ncc(Cl)c(Nc6ccc7c(c6)c6c(c(=O)n7C)OCC(F)(F)C(C7CC7)N6)n5)CC4)CC3)cc21] |
| 537a | 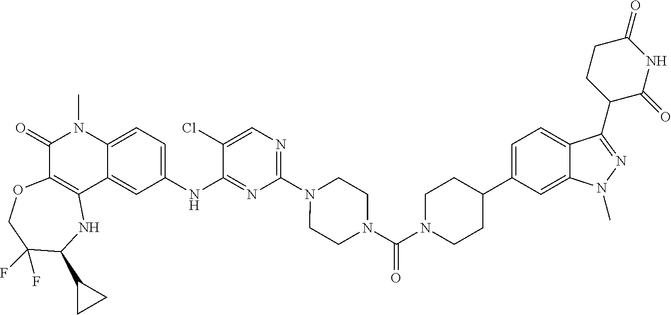 [SMILES: Cn1nc(C2CCC(=O)NC2=O)c2ccc(C3CCN(C(=O)N4CCN(c5ncc(Cl)c(Nc6ccc7c(c6)c6c(c(=O)n7C)OCC(F)(F)[C@H](C7CC7)N6)n5)CC4)CC3)cc21] |
| 538 | 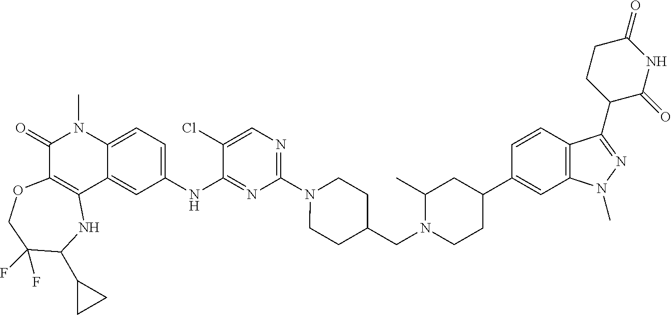 [SMILES: CC1CC(c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)C(C4CC4)N3)n2)CC1] |
| 538a | 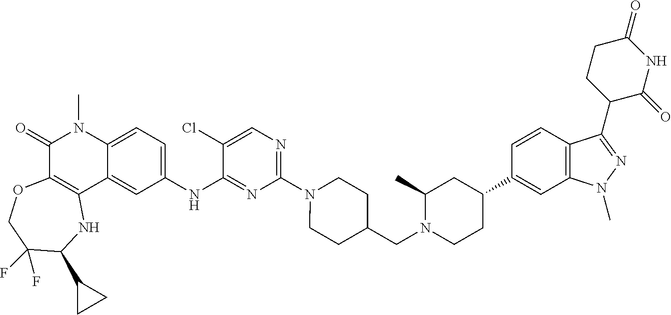 [SMILES: C[C@H]1C[C@H](c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1] |
| 538b | 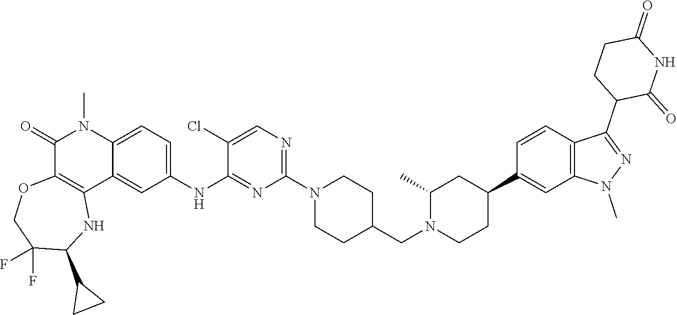 [SMILES: C[C@@H]1C[C@@H](c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1] |
| 538c | 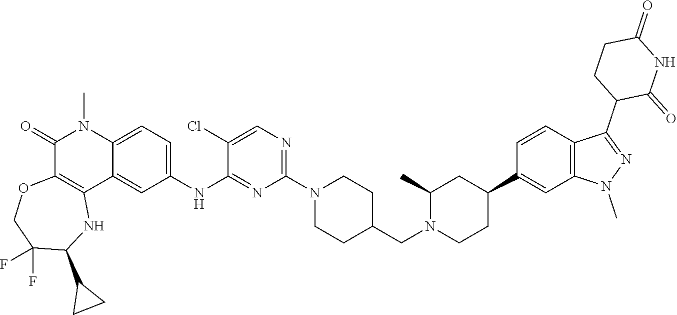 [SMILES: C[C@H]1C[C@@H](c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1] |
| 539 | 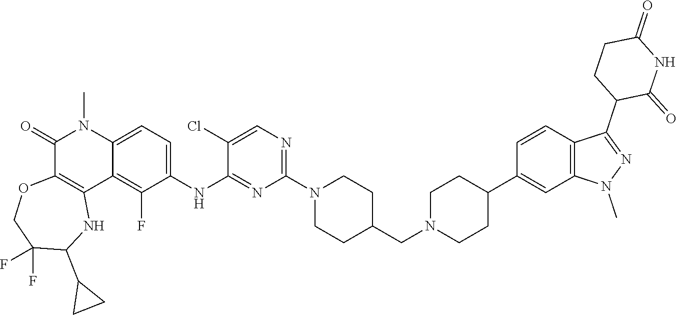 [SMILES: Cn1nc(C2CCC(=O)NC2=O)c2ccc(C3CCN(CC4CCN(c5ncc(Cl)c(Nc6ccc7c(c6F)c6c(c(=O)n7C)OCC(F)(F)C(C7CC7)N6)n5)CC4)CC3)cc21] |
| 539a | 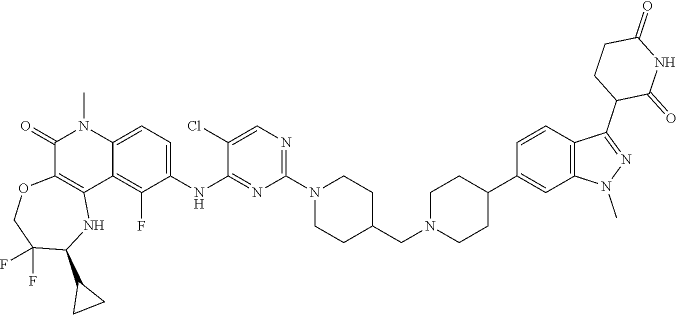 [SMILES: Cn1nc(C2CCC(=O)NC2=O)c2ccc(C3CCN(CC4CCN(c5ncc(Cl)c(Nc6ccc7c(c6F)c6c(c(=O)n7C)OCC(F)(F)[C@H](C7CC7)N6)n5)CC4)CC3)cc21] |
| 540 | 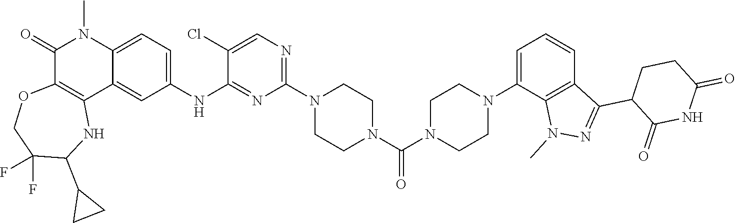 [SMILES: Cn1nc(C2CCC(=O)NC2=O)c2cccc(N3CCN(C(=O)N4CCN(c5ncc(Cl)c(Nc6ccc7c(c6)c6c(c(=O)n7C)OCC(F)(F)C(C7CC7)N6)n5)CC4)CC3)c21] |
| 540a | 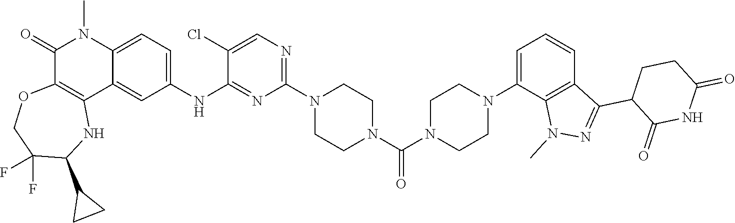 [SMILES: Cn1nc(C2CCC(=O)NC2=O)c2cccc(N3CCN(C(=O)N4CCN(c5ncc(Cl)c(Nc6ccc7c(c6)c6c(c(=O)n7C)OCC(F)(F)[C@H](C7CC7)N6)n5)CC4)CC3)c21] |
| 541 | 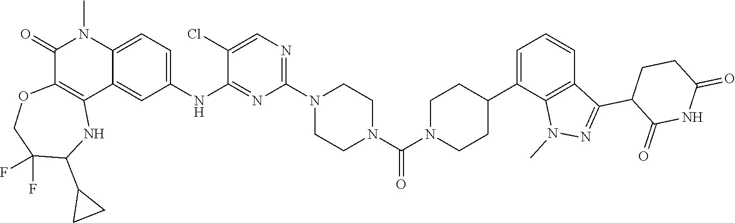 [SMILES: Cn1nc(C2CCC(=O)NC2=O)c2cccc(C3CCN(C(=O)N4CCN(c5ncc(Cl)c(Nc6ccc7c(c6)c6c(c(=O)n7C)OCC(F)(F)C(C7CC7)N6)n5)CC4)CC3)c21] |
| 541a | 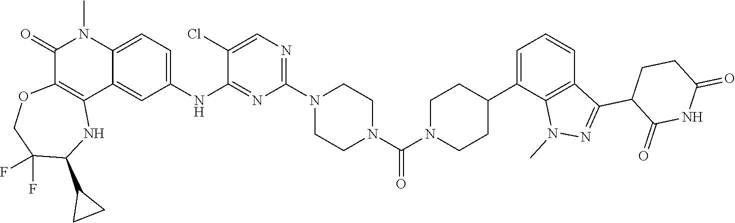 [SMILES: Cn1nc(C2CCC(=O)NC2=O)c2cccc(C3CCN(C(=O)N4CCN(c5ncc(Cl)c(Nc6ccc7c(c6)c6c(c(=O)n7C)OCC(F)(F)[C@H](C7CC7)N6)n5)CC4)CC3)c21] |
| 542 | 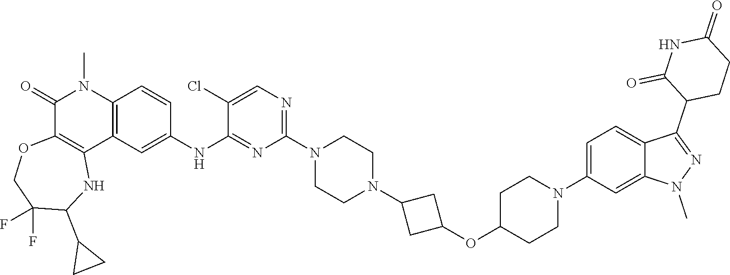 [SMILES: Cn1nc(C2CCC(=O)NC2=O)c2ccc(N3CCC(OC4CC(N5CCN(c6ncc(Cl)c(Nc7ccc8c(c7)c7c(c(=O)n8C)OCC(F)(F)C(C8CC8)N7)n6)CC5)C4)CC3)cc21] |
| 542a | 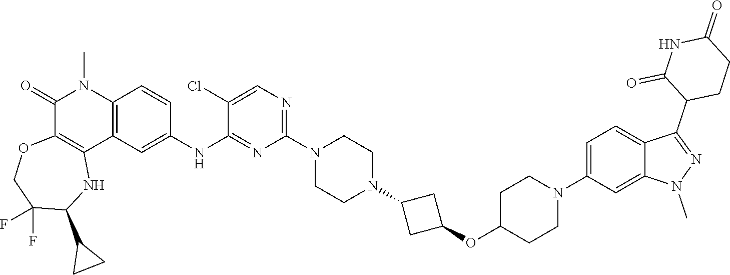 [SMILES: Cn1nc(C2CCC(=O)NC2=O)c2ccc(N3CCC(O[C@H]4C[C@H](N5CCN(c6ncc(Cl)c(Nc7ccc8c(c7)c7c(c(=O)n8C)OCC(F)(F)[C@H](C8CC8)N7)n6)CC5)C4)CC3)cc21] |
| 543 | 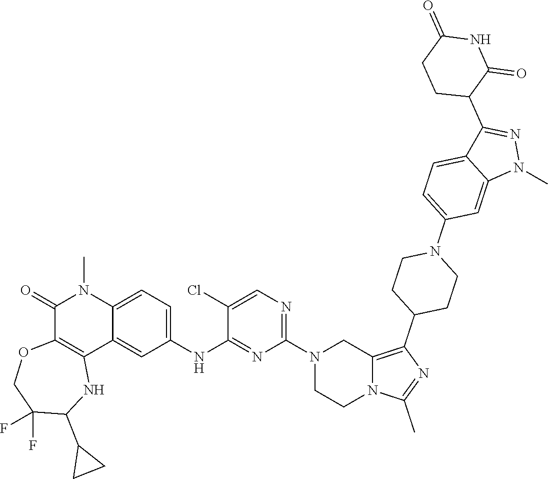 [SMILES: Cc1nc(C2CCN(c3ccc4c(C5CCC(=O)NC5=O)nn(C)c4c3)CC2)c2n1CCN(c1ncc(Cl)c(Nc3ccc4c(c3)c3c(c(=O)n4C)OCC(F)(F)C(C4CC4)N3)n1)C2] |
| 543a | 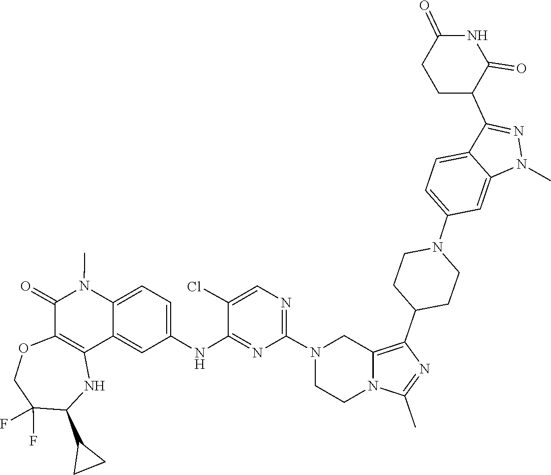 [SMILES: Cc1nc(C2CCN(c3ccc4c(C5CCC(=O)NC5=O)nn(C)c4c3)CC2)c2n1CCN(c1ncc(Cl)c(Nc3ccc4c(c3)c3c(c(=O)n4C)OCC(F)(F)[C@H](C4CC4)N3)n1)C2] |
| 544 | 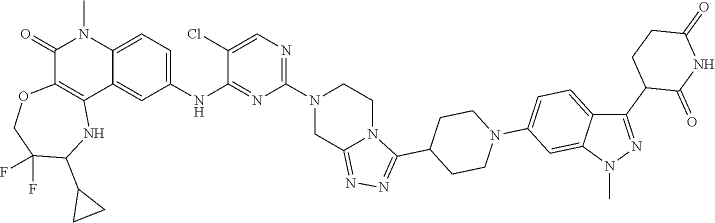 [SMILES: Cn1nc(C2CCC(=O)NC2=O)c2ccc(N3CCC(c4nnc5n4CCN(c4ncc(Cl)c(Nc6ccc7c(c6)c6c(c(=O)n7C)OCC(F)(F)C(C7CC7)N6)n4)C5)CC3)cc21] |
| 544a | 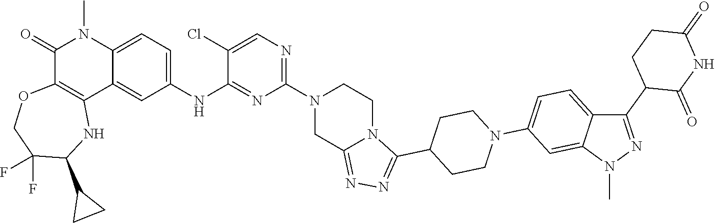 [SMILES: Cn1nc(C2CCC(=O)NC2=O)c2ccc(N3CCC(c4nnc5n4CCN(c4ncc(Cl)c(Nc6ccc7c(c6)c6c(c(=O)n7C)OCC(F)(F)[C@H](C7CC7)N6)n4)C5)CC3)cc21] |
| 545 | 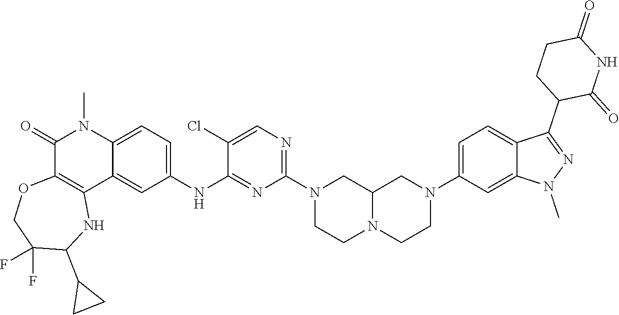 [SMILES: Cn1nc(C2CCC(=O)NC2=O)c2ccc(N3CCN4CCN(c5ncc(Cl)c(Nc6ccc7c(c6)c6c(c(=O)n7C)OCC(F)(F)C(C7CC7)N6)n5)CC4C3)cc21] |
| 545a | 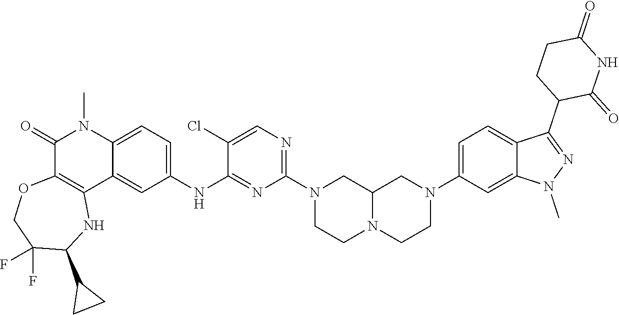 [SMILES: Cn1nc(C2CCC(=O)NC2=O)c2ccc(N3CCN4CCN(c5ncc(Cl)c(Nc6ccc7c(c6)c6c(c(=O)n7C)OCC(F)(F)[C@H](C7CC7)N6)n5)CC4C3)cc21] |
| 546 | 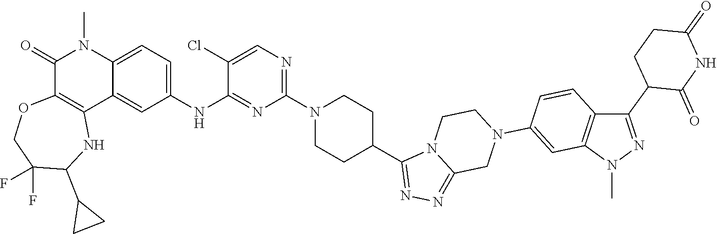 [SMILES: Cn1nc(C2CCC(=O)NC2=O)c2ccc(N3CCn4c(nnc4C4CCN(c5ncc(Cl)c(Nc6ccc7c(c6)c6c(c(=O)n7C)OCC(F)(F)C(C7CC7)N6)n5)CC4)C3)cc21] |
| 546a | 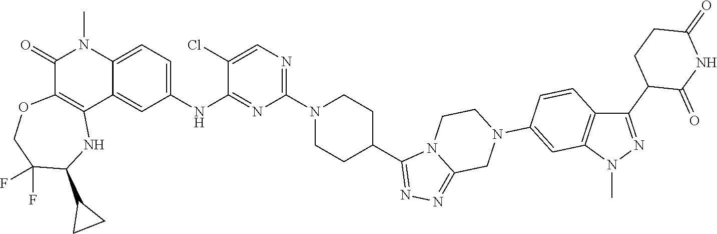 [SMILES: Cn1nc(C2CCC(=O)NC2=O)c2ccc(N3CCn4c(nnc4C4CCN(c5ncc(Cl)c(Nc6ccc7c(c6)c6c(c(=O)n7C)OCC(F)(F)[C@H](C7CC7)N6)n5)CC4)C3)cc21] |
| 547 | 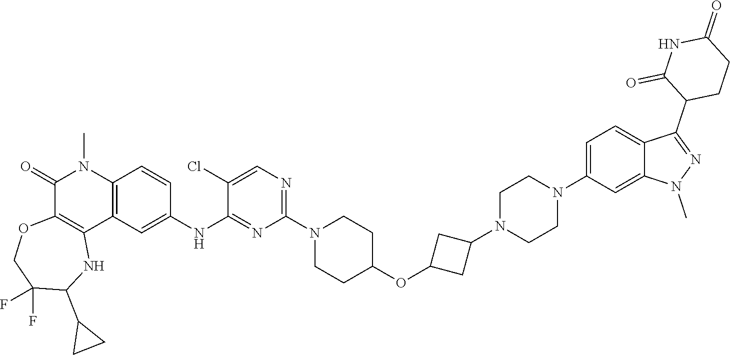 [SMILES: Cn1nc(C2CCC(=O)NC2=O)c2ccc(N3CCN(C4CC(OC5CCN(c6ncc(Cl)c(Nc7ccc8c(c7)c7c(c(=O)n8C)OCC(F)(F)C(C8CC8)N7)n6)CC5)C4)CC3)cc21] |
| 547a | 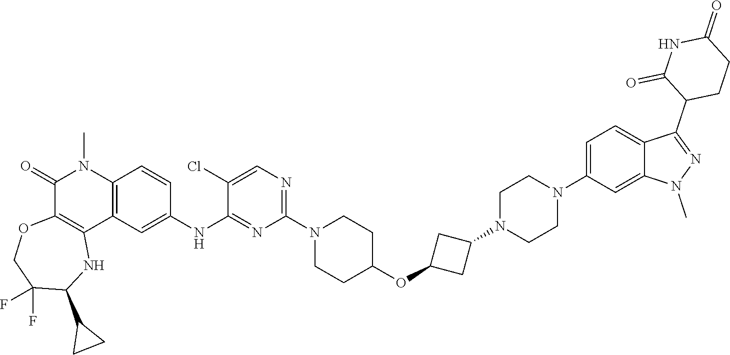 [SMILES: Cn1nc(C2CCC(=O)NC2=O)c2ccc(N3CCN([C@H]4C[C@H](OC5CCN(c6ncc(Cl)c(Nc7ccc8c(c7)c7c(c(=O)n8C)OCC(F)(F)[C@H](C8CC8)N7)n6)CC5)C4)CC3)cc21] |
| 548 | 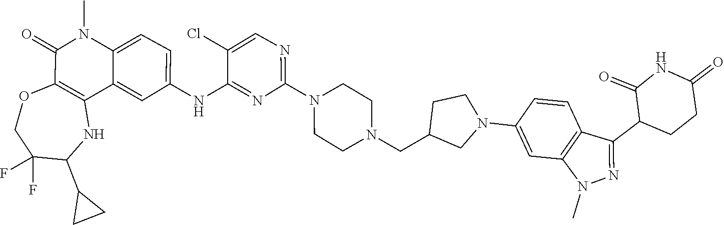 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)C(C7CC7)N6)n5)CC4)C3)cc21] |
| 548a | 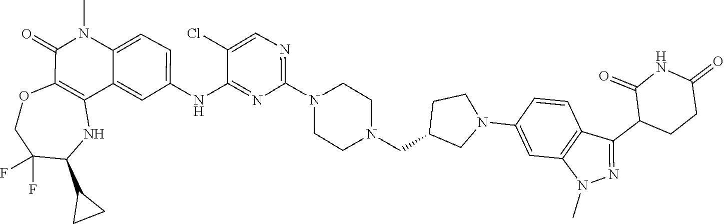 [SMILES: Cn1nc(C2CCC(=O)NC2=O)c2ccc(N3CC[C@@H](CN4CCN(c5ncc(Cl)c(Nc6ccc7c(c6)c6c(c(=O)n7C)OCC(F)(F)[C@H](C7CC7)N6)n5)CC4)C3)cc21] |
| 548b | 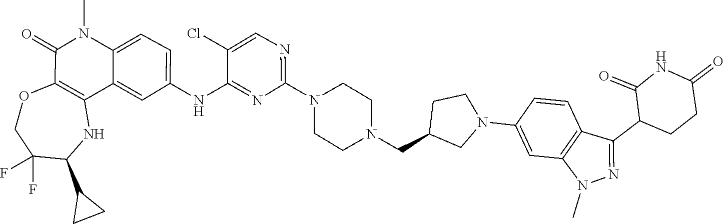 [SMILES: Cn1nc(C2CCC(=O)NC2=O)c2ccc(N3CC[C@H](CN4CCN(c5ncc(Cl)c(Nc6ccc7c(c6)c6c(c(=O)n7C)OCC(F)(F)[C@H](C7CC7)N6)n5)CC4)C3)cc21] |
| 549 | 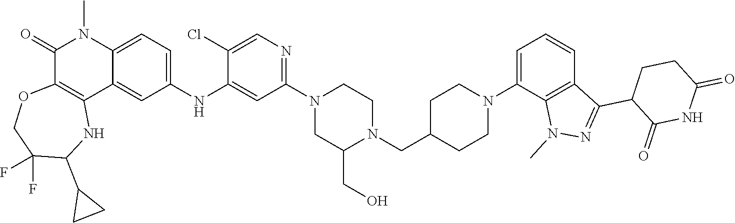 [SMILES: Cn1nc(C2CCC(=O)NC2=O)c2cccc(N3CCC(CN4CCN(c5cc(Nc6ccc7c(c6)c6c(c(=O)n7C)OCC(F)(F)C(C7CC7)N6)c(Cl)cn5)CC4CO)CC3)c21] |
| 549a | 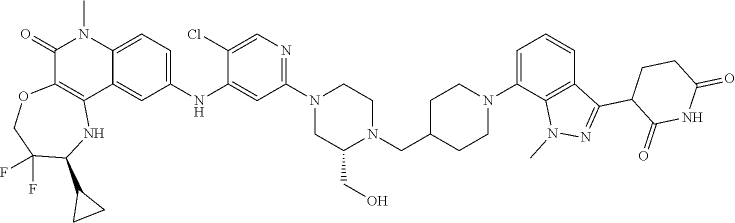 [SMILES: Cn1nc(C2CCC(=O)NC2=O)c2cccc(N3CCC(CN4CCN(c5cc(Nc6ccc7c(c6)c6c(c(=O)n7C)OCC(F)(F)[C@H](C7CC7)N6)c(Cl)cn5)C[C@H]4CO)CC3)c21] |
| 550 | 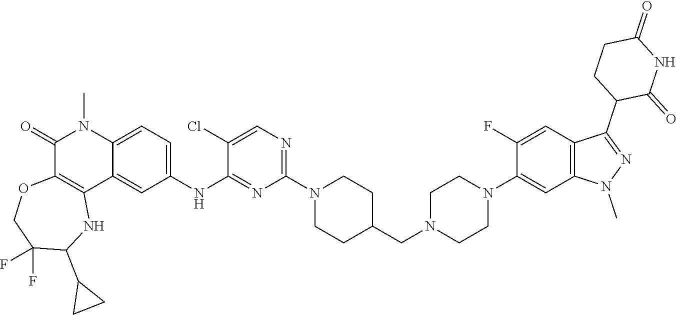 [SMILES: Cn1nc(C2CCC(=O)NC2=O)c2cc(F)c(N3CCN(CC4CCN(c5ncc(Cl)c(Nc6ccc7c(c6)c6c(c(=O)n7C)OCC(F)(F)C(C7CC7)N6)n5)CC4)CC3)cc21] |
| 550a | 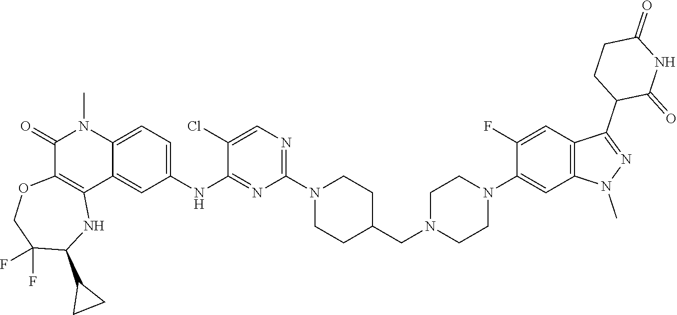 [SMILES: Cn1nc(C2CCC(=O)NC2=O)c2cc(F)c(N3CCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 551 | 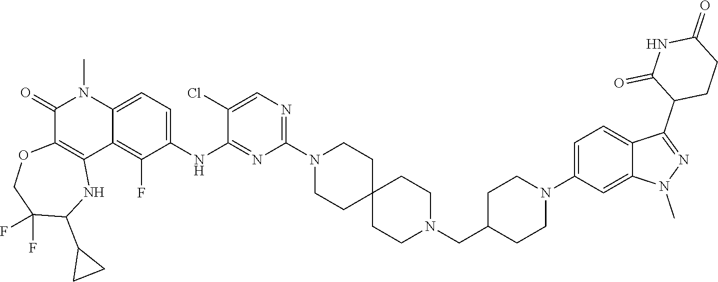 [SMILES: Cn1nc(C2CCC(=O)NC2=O)c2ccc(N3CCC(CN4CCC5(CC4)CCN(c4ncc(Cl)c(Nc6ccc7c(c6F)c6c(c(=O)n7C)OCC(F)(F)C(C7CC7)N6)n4)CC5)CC3)cc21] |
| 551a | 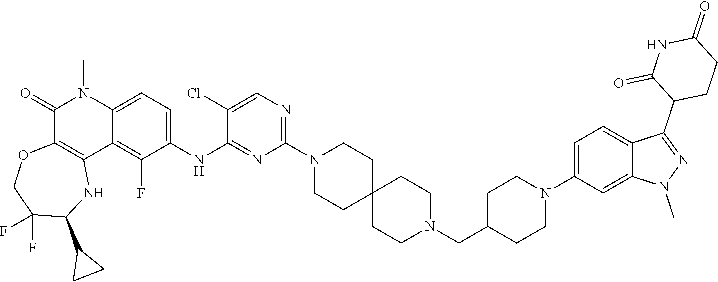 [SMILES: Cn1nc(C2CCC(=O)NC2=O)c2ccc(N3CCC(CN4CCC5(CC4)CCN(c4ncc(Cl)c(Nc6ccc7c(c6F)c6c(c(=O)n7C)OCC(F)(F)[C@H](C7CC7)N6)n4)CC5)CC3)cc21] |
| 552 | 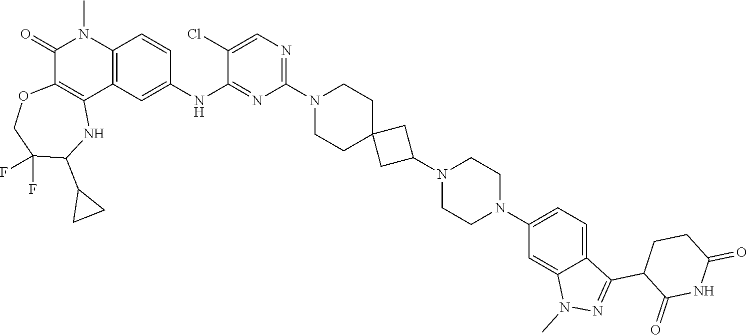 [SMILES: Cn1nc(C2CCC(=O)NC2=O)c2ccc(N3CCN(C4CC5(CCN(c6ncc(Cl)c(Nc7ccc8c(c7)c7c(c(=O)n8C)OCC(F)(F)C(C8CC8)N7)n6)CC5)C4)CC3)cc21] |
| 552a | 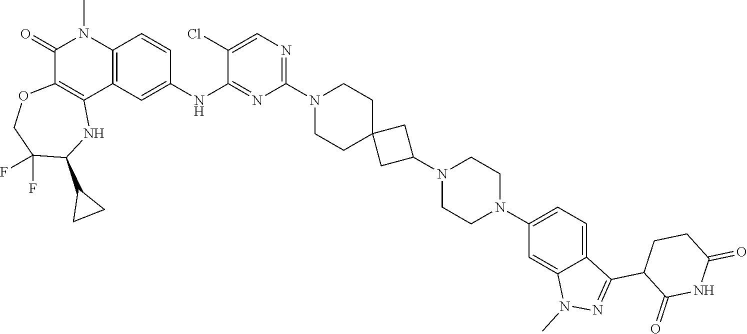 [SMILES: Cn1nc(C2CCC(=O)NC2=O)c2ccc(N3CCN(C4CC5(CCN(c6ncc(Cl)c(Nc7ccc8c(c7)c7c(c(=O)n8C)OCC(F)(F)[C@H](C8CC8)N7)n6)CC5)C4)CC3)cc21] |
| 553 | 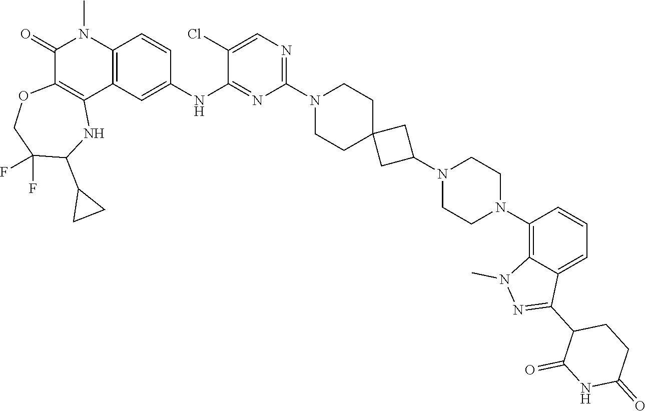 [SMILES: Cn1nc(C2CCC(=O)NC2=O)c2cccc(N3CCN(C4CC5(CCN(c6ncc(Cl)c(Nc7ccc8c(c7)c7c(c(=O)n8C)OCC(F)(F)C(C8CC8)N7)n6)CC5)C4)CC3)c21] |
| 553a | 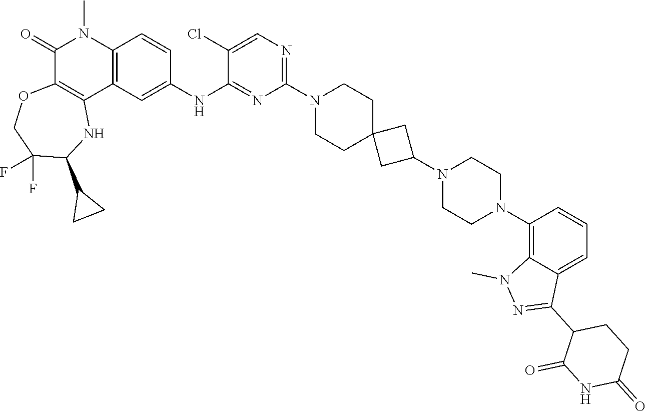 [SMILES: Cn1nc(C2CCC(=O)NC2=O)c2cccc(N3CCN(C4CC5(CCN(c6ncc(Cl)c(Nc7ccc8c(c7)c7c(c(=O)n8C)OCC(F)(F)[C@H](C8CC8)N7)n6)CC5)C4)CC3)c21] |
| 554 | 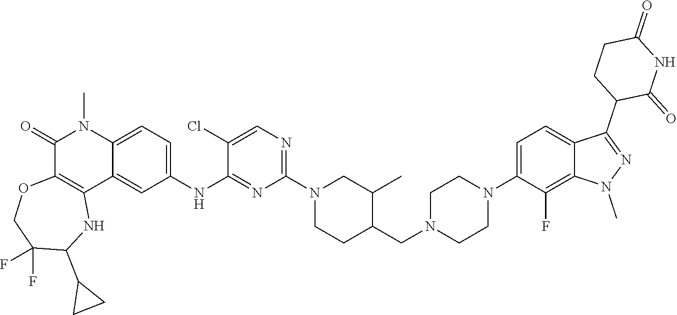 [SMILES: CC1CN(c2ncc(Cl)c(Nc3ccc4c(c3)c3c(c(=O)n4C)OCC(F)(F)C(C4CC4)N3)n2)CCC1CN1CCN(c2ccc3c(C4CCC(=O)NC4=O)nn(C)c3c2F)CC1] |
| 554a | 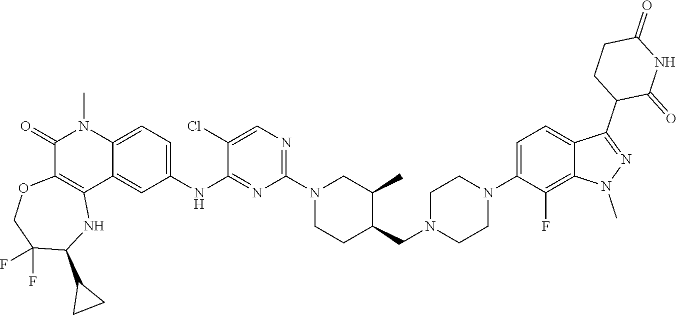 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCN(c2ccc3c(C4CCC(=O)NC4=O)nn(C)c3c2F)CC1] |
| 555 | 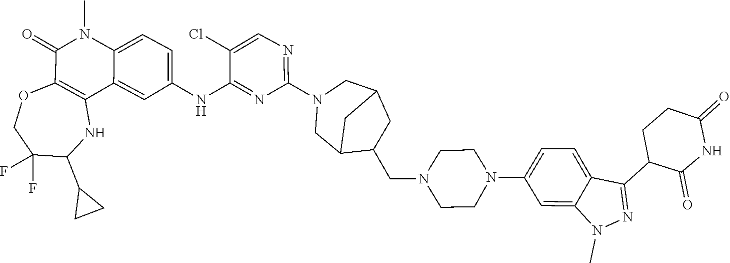 [SMILES: Cn1nc(C2CCC(=O)NC2=O)c2ccc(N3CCN(CC4CC5CC4CN(c4ncc(Cl)c(Nc6ccc7c(c6)c6c(c(=O)n7C)OCC(F)(F)C(C7CC7)N6)n4)C5)CC3)cc21] |
| 555a | 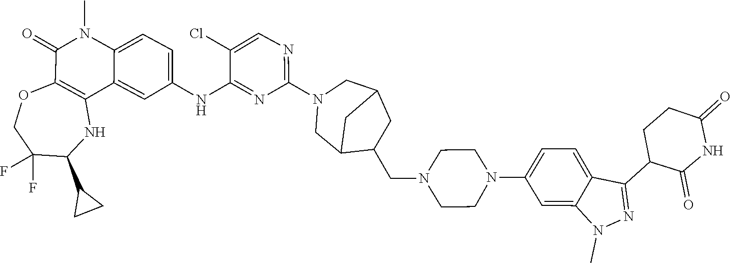 [SMILES: Cn1nc(C2CCC(=O)NC2=O)c2ccc(N3CCN(CC4CC5CC4CN(c4ncc(Cl)c(Nc6ccc7c(c6)c6c(c(=O)n7C)OCC(F)(F)[C@H](C7CC7)N6)n4)C5)CC3)cc21] |
| 556 | 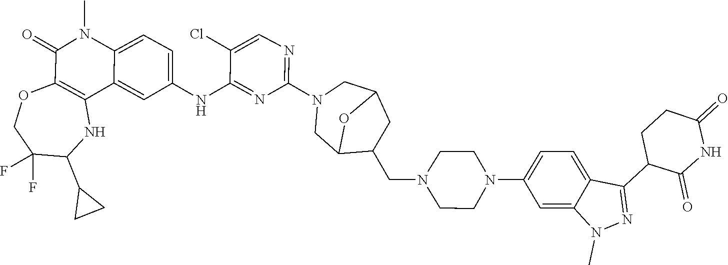 [SMILES: Cn1nc(C2CCC(=O)NC2=O)c2ccc(N3CCN(CC4CC5CN(c6ncc(Cl)c(Nc7ccc8c(c7)c7c(c(=O)n8C)OCC(F)(F)C(C8CC8)N7)n6)CC4O5)CC3)cc21] |
| 556a | 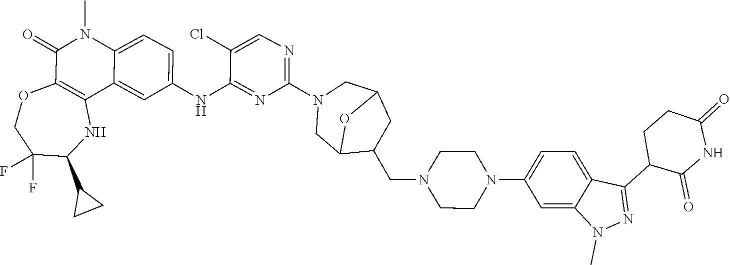 [SMILES: Cn1nc(C2CCC(=O)NC2=O)c2ccc(N3CCN(CC4CC5CN(c6ncc(Cl)c(Nc7ccc8c(c7)c7c(c(=O)n8C)OCC(F)(F)[C@H](C8CC8)N7)n6)CC4O5)CC3)cc21] |
| 557 | 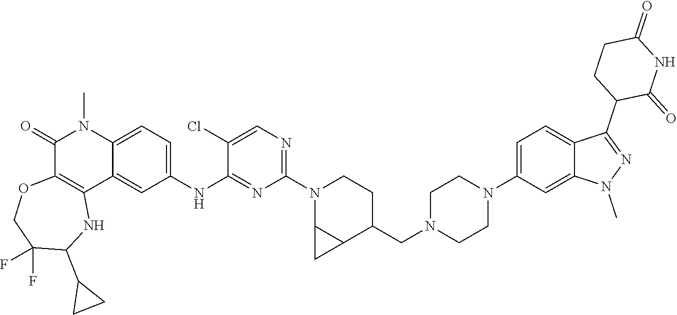 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)C5CC45)CC3)cc21] |
| 557a | 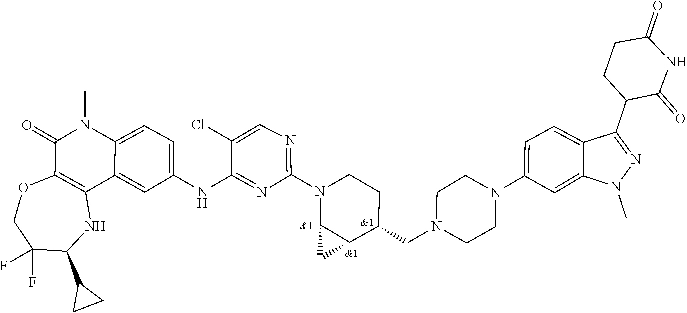 [SMILES: Cn1nc(C2CCC(=O)NC2=O)c2ccc(N3CCN(C[C@H]4CCN(c5ncc(Cl)c(Nc6ccc7c(c6)c6c(c(=O)n7C)OCC(F)(F)[C@H](C7CC7)N6)n5)[C@H]5C[C@@H]45)CC3)cc21] |
| 558 | 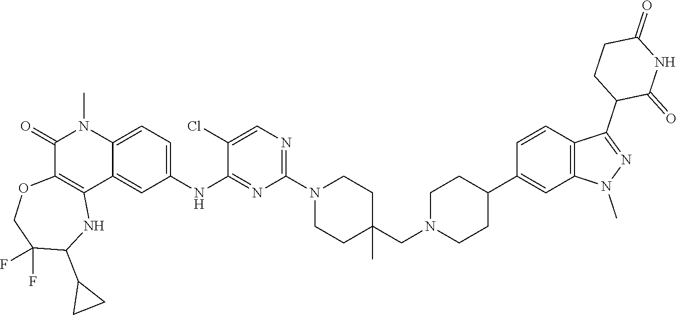 [SMILES: Cn1nc(C2CCC(=O)NC2=O)c2ccc(C3CCN(CC4(C)CCN(c5ncc(Cl)c(Nc6ccc7c(c6)c6c(c(=O)n7C)OCC(F)(F)C(C7CC7)N6)n5)CC4)CC3)cc21] |
| 558a | 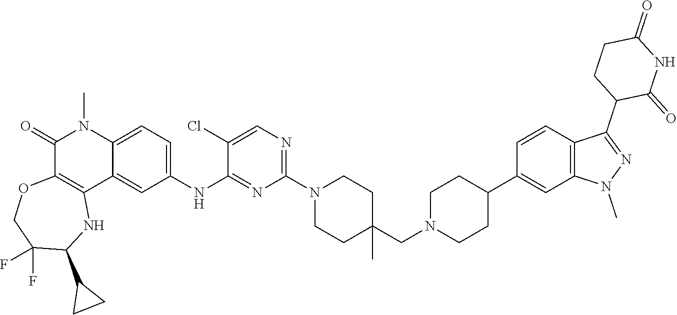 [SMILES: Cn1nc(C2CCC(=O)NC2=O)c2ccc(C3CCN(CC4(C)CCN(c5ncc(Cl)c(Nc6ccc7c(c6)c6c(c(=O)n7C)OCC(F)(F)[C@H](C7CC7)N6)n5)CC4)CC3)cc21] |
| 559 | 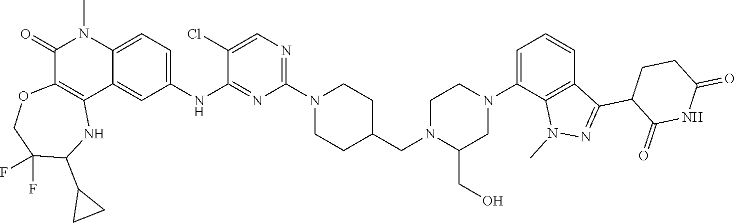 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)C(C7CC7)N6)n5)CC4)C(CO)C3)c21] |
| 559a | 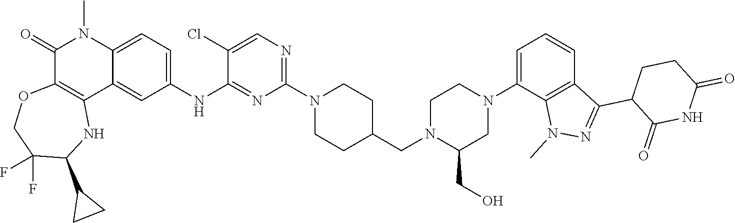 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)[C@H](C7CC7)N6)n5)CC4)[C@H](CO)C3)c21] |
| 559b | 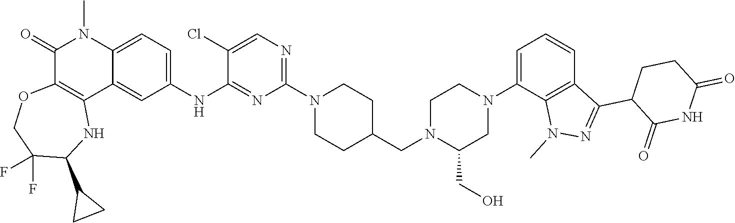 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)[C@H](C7CC7)N6)n5)CC4)[C@@H](CO)C3)c21] |
| 560 | 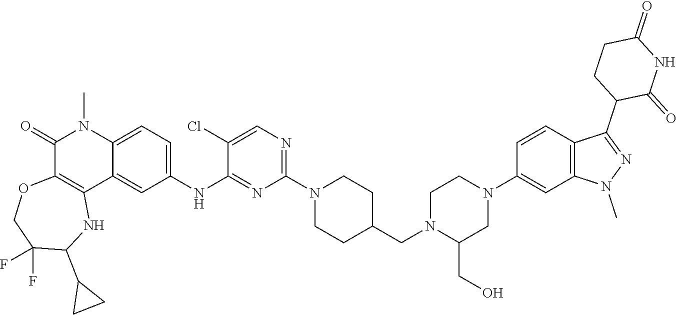 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)CC4)C(CO)C3)cc21] |
| 560a | 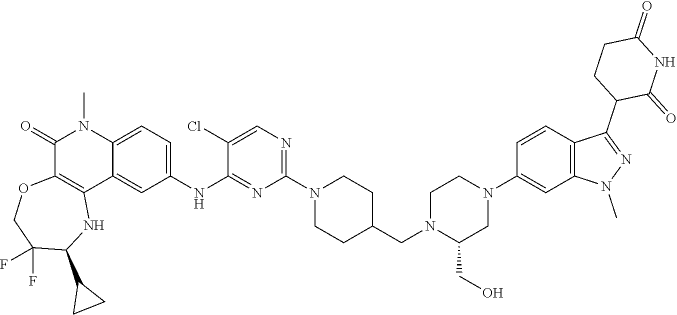 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)[C@@H](CO)C3)cc21] |
| 560b | 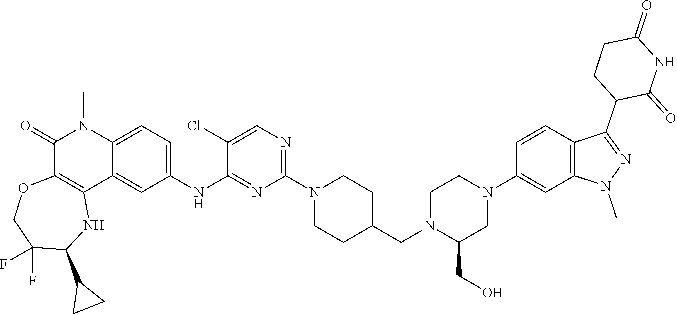 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)[C@H](CO)C3)cc21] |
| 561 | 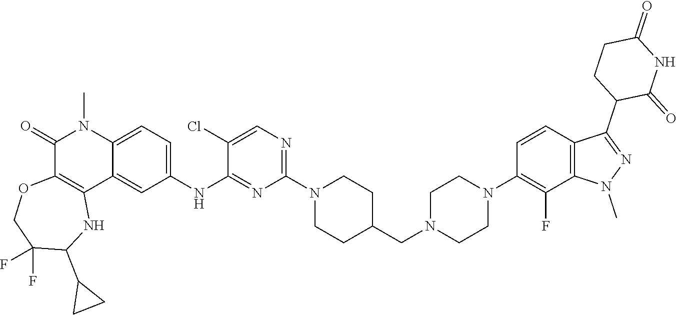 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)CC4)CC3)c(F)c21] |
| 561a | 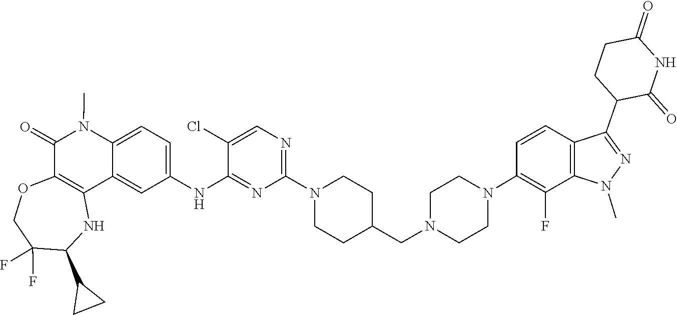 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)CC3)c(F)c21] |
| 562 | 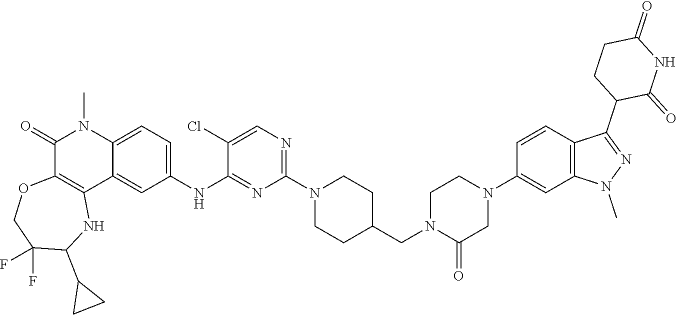 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)CC4)C(=O)C3)cc21] |
| 562a | 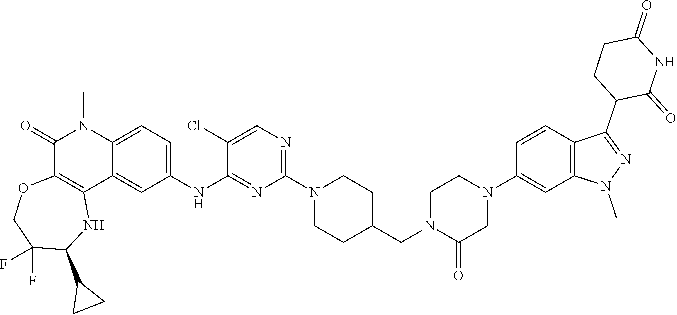 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)C(=O)C3)cc21] |
| 563 | 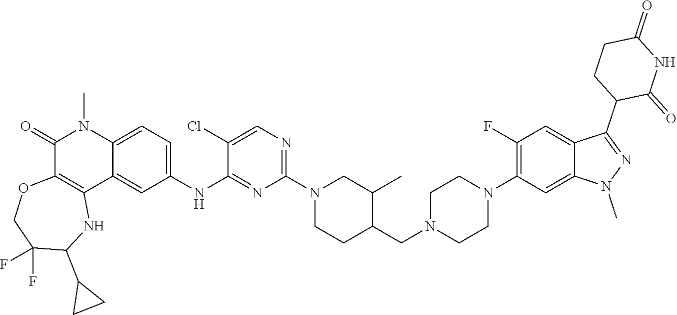 [SMILES: CC1CN(c2ncc(Cl)c(Nc3ccc4c(c3)c3c(c(=O)n4C)OCC(F)(F)C(C4CC4)N3)n2)CCC1CN1CCN(c2cc3c(cc2F)c(C2CCC(=O)NC2=O)nn3C)CC1] |
| 563a | 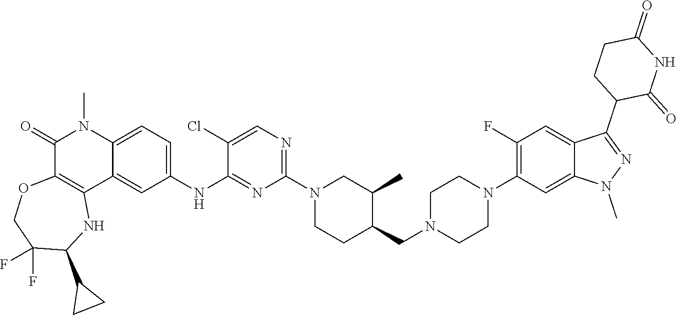 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCN(c2cc3c(cc2F)c(C2CCC(=O)NC2=O)nn3C)CC1] |
| 563b | 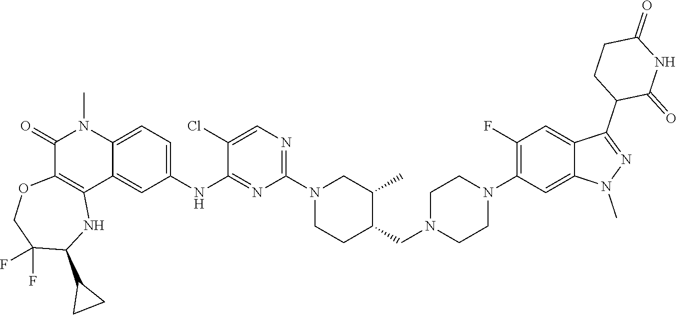 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCN(c2cc3c(cc2F)c(C2CCC(=O)NC2=O)nn3C)CC1] |
| 564 | 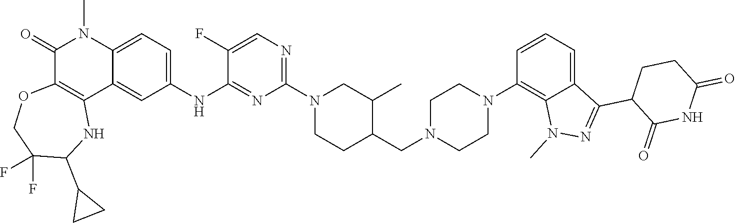 [SMILES: CC1CN(c2ncc(F)c(Nc3ccc4c(c3)c3c(c(=O)n4C)OCC(F)(F)C(C4CC4)N3)n2)CCC1CN1CCN(c2cccc3c(C4CCC(=O)NC4=O)nn(C)c23)CC1] |
| 564a | 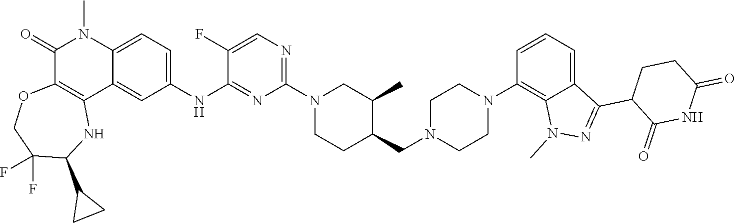 [SMILES: C[C@@H]1CN(c2ncc(F)c(Nc3ccc4c(c3)c3c(c(=O)n4C)OCC(F)(F)[C@H](C4CC4)N3)n2)CC[C@@H]1CN1CCN(c2cccc3c(C4CCC(=O)NC4=O)nn(C)c23)CC1] |
| 565 | 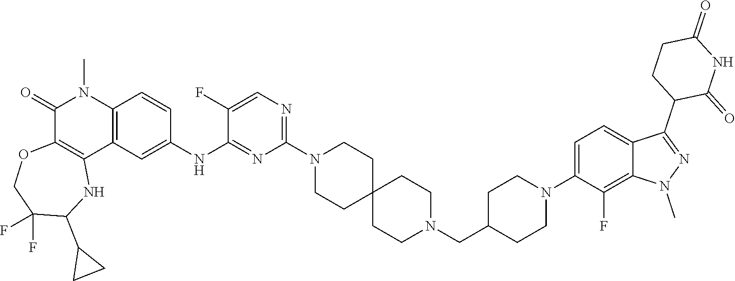 [SMILES: Cn1nc(C2CCC(=O)NC2=O)c2ccc(N3CCC(CN4CCC5(CC4)CCN(c4ncc(F)c(Nc6ccc7c(c6)c6c(c(=O)n7C)OCC(F)(F)C(C7CC7)N6)n4)CC5)CC3)c(F)c21] |
| 565a | 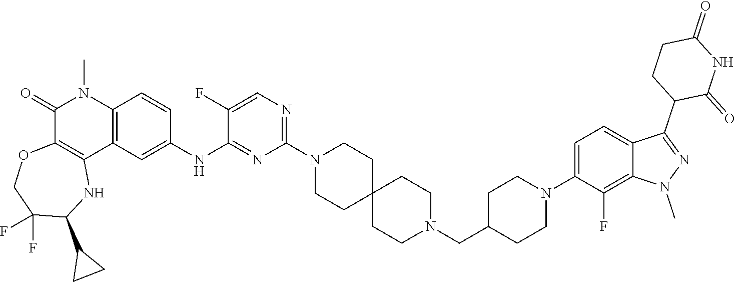 [SMILES: Cn1nc(C2CCC(=O)NC2=O)c2ccc(N3CCC(CN4CCC5(CC4)CCN(c4ncc(F)c(Nc6ccc7c(c6)c6c(c(=O)n7C)OCC(F)(F)[C@H](C7CC7)N6)n4)CC5)CC3)c(F)c21] |
| 566 | 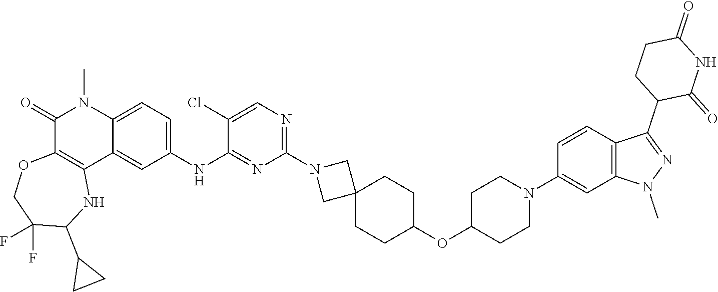 [SMILES: Cn1nc(C2CCC(=O)NC2=O)c2ccc(N3CCC(OC4CCC5(CC4)CN(c4ncc(Cl)c(Nc6ccc7c(c6)c6c(c(=O)n7C)OCC(F)(F)C(C7CC7)N6)n4)C5)CC3)cc21] |
| 566a | 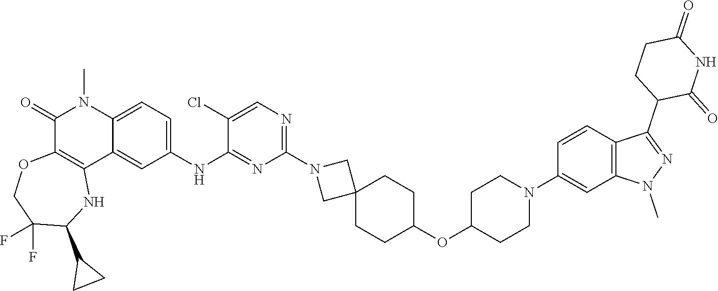 [SMILES: Cn1nc(C2CCC(=O)NC2=O)c2ccc(N3CCC(OC4CCC5(CC4)CN(c4ncc(Cl)c(Nc6ccc7c(c6)c6c(c(=O)n7C)OCC(F)(F)[C@H](C7CC7)N6)n4)C5)CC3)cc21] |
| 568 | 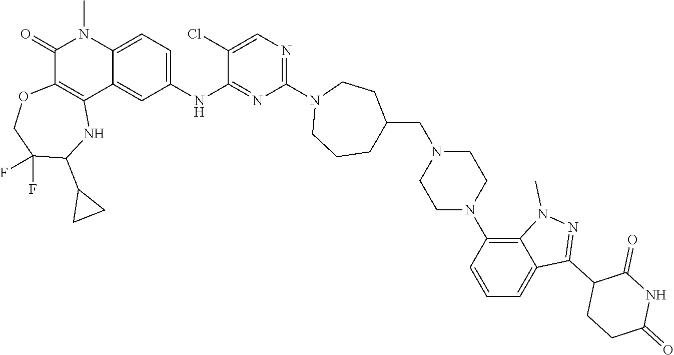 [SMILES: Cn1nc(C2CCC(=O)NC2=O)c2cccc(N3CCN(CC4CCCN(c5ncc(Cl)c(Nc6ccc7c(c6)c6c(c(=O)n7C)OCC(F)(F)C(C7CC7)N6)n5)CC4)CC3)c21] |
| 568a | 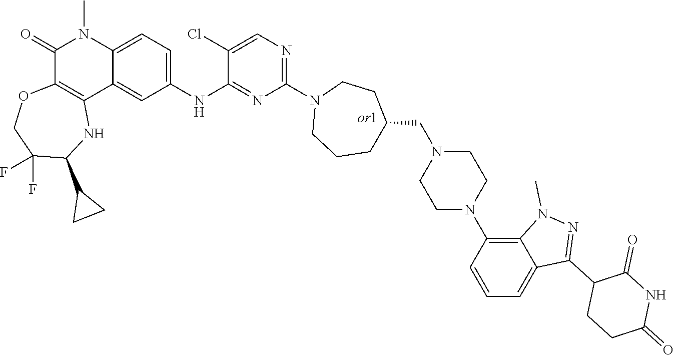 [SMILES: Cn1nc(C2CCC(=O)NC2=O)c2cccc(N3CCN(C[C@@H]4CCCN(c5ncc(Cl)c(Nc6ccc7c(c6)c6c(c(=O)n7C)OCC(F)(F)[C@H](C7CC7)N6)n5)CC4)CC3)c21] |
| 568b | 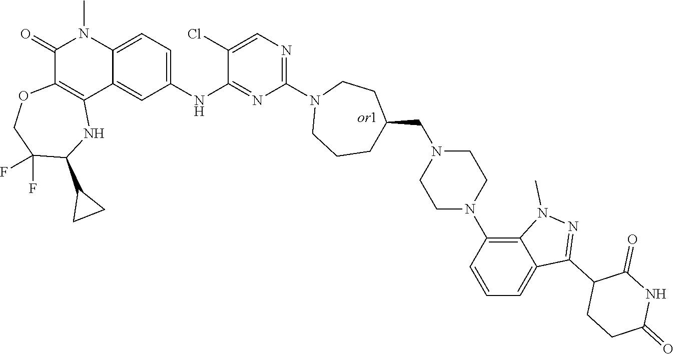 [SMILES: Cn1nc(C2CCC(=O)NC2=O)c2cccc(N3CCN(C[C@H]4CCCN(c5ncc(Cl)c(Nc6ccc7c(c6)c6c(c(=O)n7C)OCC(F)(F)[C@H](C7CC7)N6)n5)CC4)CC3)c21] |
| 569 | 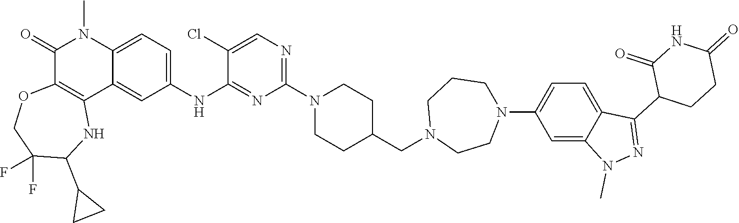 [SMILES: Cn1nc(C2CCC(=O)NC2=O)c2ccc(N3CCCN(CC4CCN(c5ncc(Cl)c(Nc6ccc7c(c6)c6c(c(=O)n7C)OCC(F)(F)C(C7CC7)N6)n5)CC4)CC3)cc21] |
| 569a | 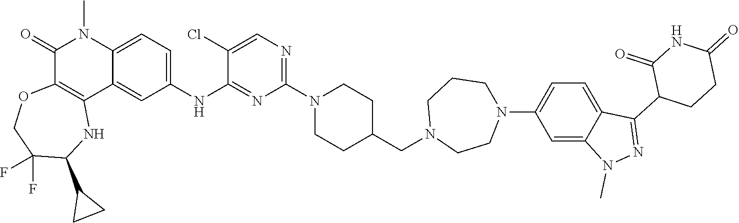 [SMILES: Cn1nc(C2CCC(=O)NC2=O)c2ccc(N3CCCN(CC4CCN(c5ncc(Cl)c(Nc6ccc7c(c6)c6c(c(=O)n7C)OCC(F)(F)[C@H](C7CC7)N6)n5)CC4)CC3)cc21] |
| 570 | 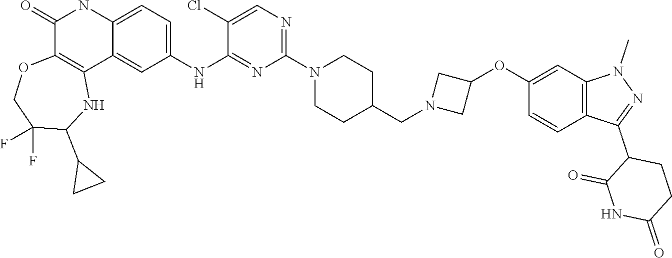 [SMILES: Cn1nc(C2CCC(=O)NC2=O)c2ccc(OC3CN(CC4CCN(c5ncc(Cl)c(Nc6ccc7[nH]c(=O)c8c(c7c6)NC(C6CC6)C(F)(F)CO8)n5)CC4)C3)cc21] |
| 570a | 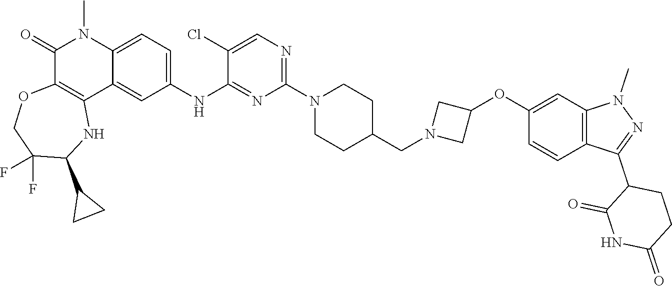 [SMILES: Cn1nc(C2CCC(=O)NC2=O)c2ccc(OC3CN(CC4CCN(c5ncc(Cl)c(Nc6ccc7c(c6)c6c(c(=O)n7C)OCC(F)(F)[C@H](C7CC7)N6)n5)CC4)C3)cc21] |
| 572 | 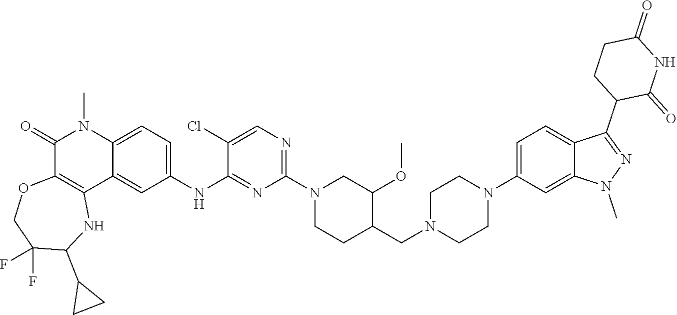 [SMILES: COC1CN(c2ncc(Cl)c(Nc3ccc4c(c3)c3c(c(=O)n4C)OCC(F)(F)C(C4CC4)N3)n2)CCC1CN1CCN(c2ccc3c(C4CCC(=O)NC4=O)nn(C)c3c2)CC1] |
| 572a | 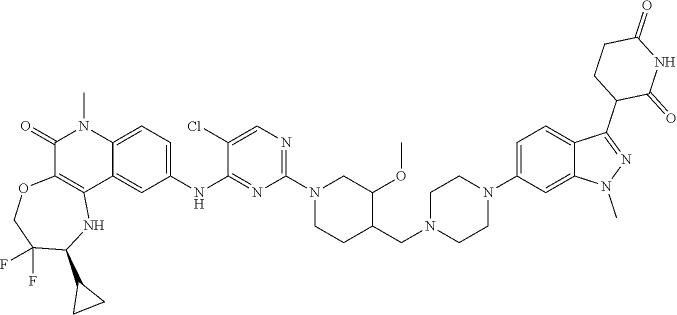 [SMILES: COC1CN(c2ncc(Cl)c(Nc3ccc4c(c3)c3c(c(=O)n4C)OCC(F)(F)[C@H](C4CC4)N3)n2)CCC1CN1CCN(c2ccc3c(C4CCC(=O)NC4=O)nn(C)c3c2)CC1] |
| 573 | 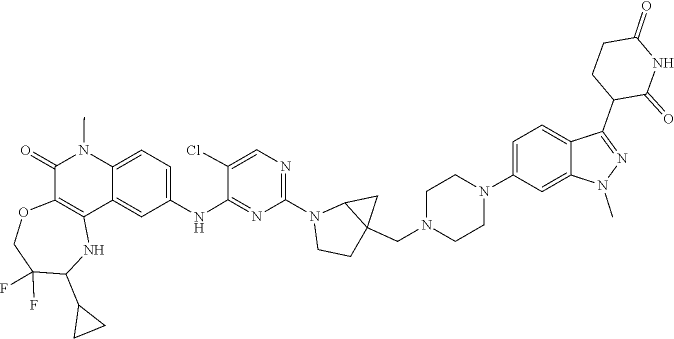 [SMILES: Cn1nc(C2CCC(=O)NC2=O)c2ccc(N3CCN(CC45CCN(c6ncc(Cl)c(Nc7ccc8c(c7)c7c(c(=O)n8C)OCC(F)(F)C(C8CC8)N7)n6)C4C5)CC3)cc21] |
| 573a | 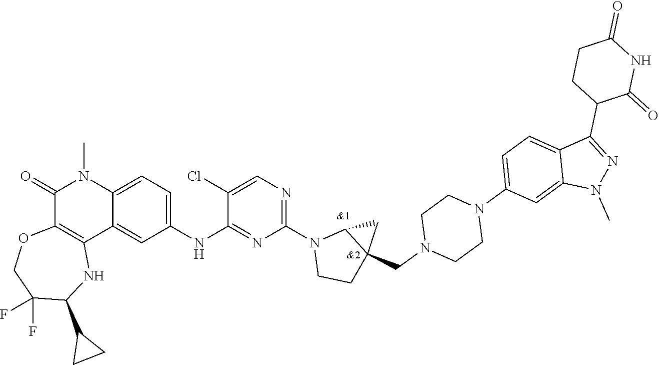 [SMILES: Cn1nc(C2CCC(=O)NC2=O)c2ccc(N3CCN(C[C@@]45CCN(c6ncc(Cl)c(Nc7ccc8c(c7)c7c(c(=O)n8C)OCC(F)(F)[C@H](C8CC8)N7)n6)[C@@H]4C5)CC3)cc21] |
| 574 | 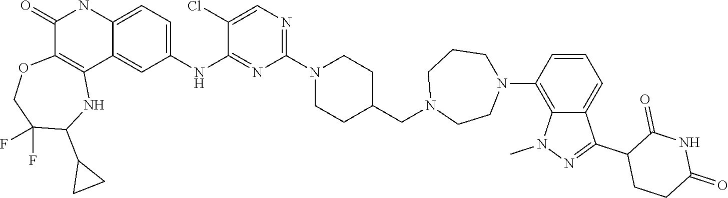 [SMILES: Cn1nc(C2CCC(=O)NC2=O)c2cccc(N3CCCN(CC4CCN(c5ncc(Cl)c(Nc6ccc7[nH]c(=O)c8c(c7c6)NC(C6CC6)C(F)(F)CO8)n5)CC4)CC3)c21] |
| 574a | 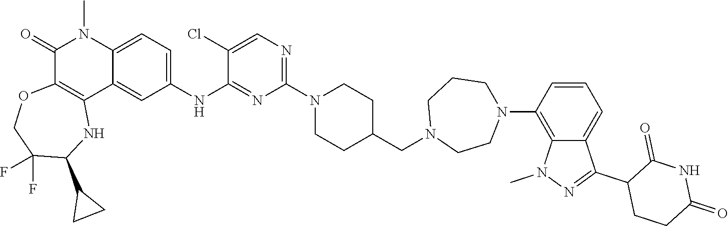 [SMILES: Cn1nc(C2CCC(=O)NC2=O)c2cccc(N3CCCN(CC4CCN(c5ncc(Cl)c(Nc6ccc7c(c6)c6c(c(=O)n7C)OCC(F)(F)[C@H](C7CC7)N6)n5)CC4)CC3)c21] |
| 575 | 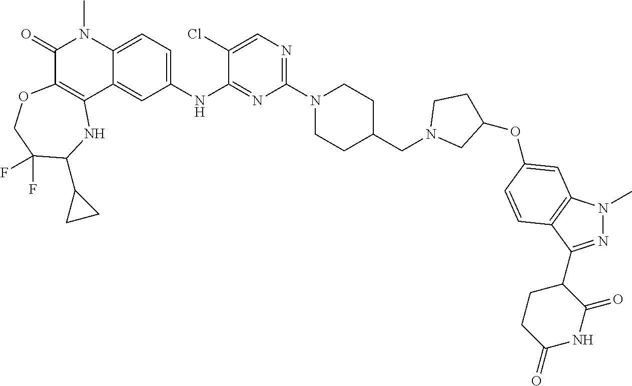 [SMILES: Cn1nc(C2CCC(=O)NC2=O)c2ccc(OC3CCN(CC4CCN(c5ncc(Cl)c(Nc6ccc7c(c6)c6c(c(=O)n7C)OCC(F)(F)C(C7CC7)N6)n5)CC4)C3)cc21] |
| 575a | 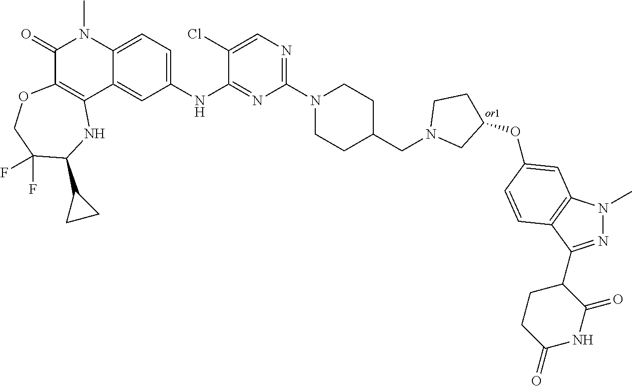 [SMILES: Cn1nc(C2CCC(=O)NC2=O)c2ccc(O[C@H]3CCN(CC4CCN(c5ncc(Cl)c(Nc6ccc7c(c6)c6c(c(=O)n7C)OCC(F)(F)[C@H](C7CC7)N6)n5)CC4)C3)cc21] |
| 575b | 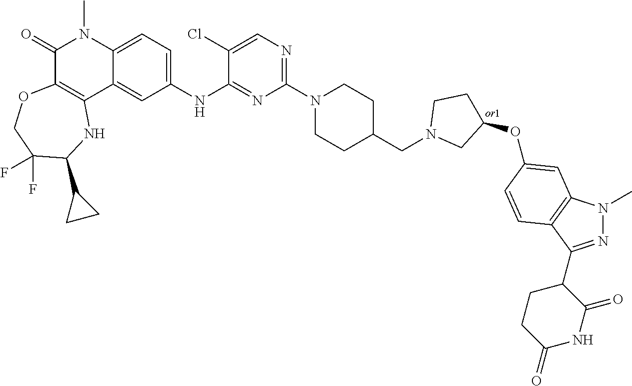 [SMILES: Cn1nc(C2CCC(=O)NC2=O)c2ccc(O[C@@H]3CCN(CC4CCN(c5ncc(Cl)c(Nc6ccc7c(c6)c6c(c(=O)n7C)OCC(F)(F)[C@H](C7CC7)N6)n5)CC4)C3)cc21] |
| 576 | 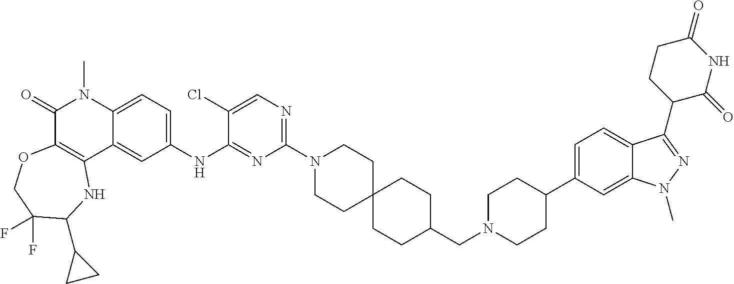 [SMILES: Cn1nc(C2CCC(=O)NC2=O)c2ccc(C3CCN(CC4CCC5(CC4)CCN(c4ncc(Cl)c(Nc6ccc7c(c6)c6c(c(=O)n7C)OCC(F)(F)C(C7CC7)N6)n4)CC5)CC3)cc21] |
| 576a | 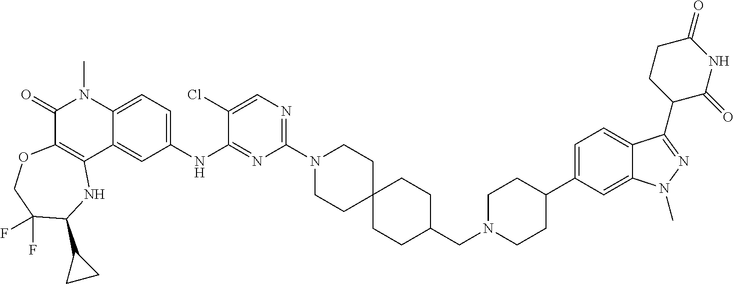 [SMILES: Cn1nc(C2CCC(=O)NC2=O)c2ccc(C3CCN(CC4CCC5(CC4)CCN(c4ncc(Cl)c(Nc6ccc7c(c6)c6c(c(=O)n7C)OCC(F)(F)[C@H](C7CC7)N6)n4)CC5)CC3)cc21] |
| 577 | 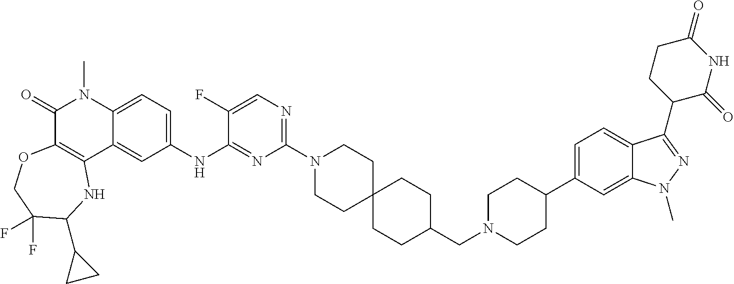 [SMILES: Cn1nc(C2CCC(=O)NC2=O)c2ccc(C3CCN(CC4CCC5(CC4)CCN(c4ncc(F)c(Nc6ccc7c(c6)c6c(c(=O)n7C)OCC(F)(F)C(C7CC7)N6)n4)CC5)CC3)cc21] |
| 577a | 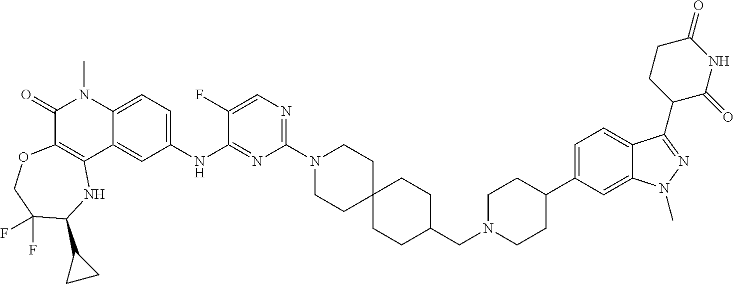 [SMILES: Cn1nc(C2CCC(=O)NC2=O)c2ccc(C3CCN(CC4CCC5(CC4)CCN(c4ncc(F)c(Nc6ccc7c(c6)c6c(c(=O)n7C)OCC(F)(F)[C@H](C7CC7)N6)n4)CC5)CC3)cc21] |
| 578 | 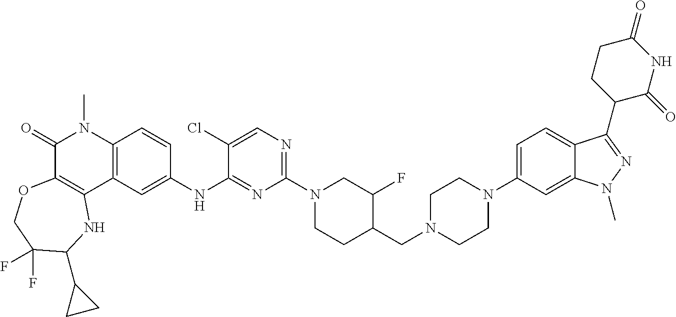 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)CC4F)CC3)cc21] |
| 578a | 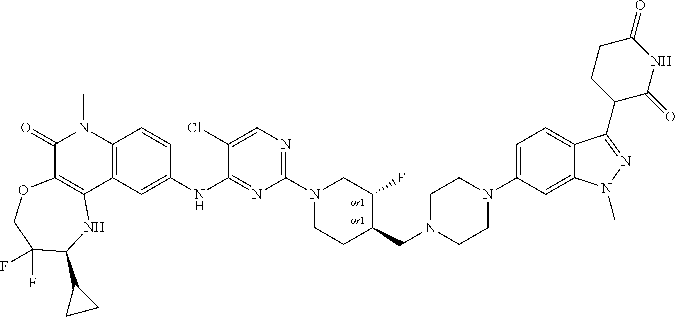 [SMILES: Cn1nc(C2CCC(=O)NC2=O)c2ccc(N3CCN(C[C@H]4CCN(c5ncc(Cl)c(Nc6ccc7c(c6)c6c(c(=O)n7C)OCC(F)(F)[C@H](C7CC7)N6)n5)C[C@@H]4F)CC3)cc21] |
| 578b | 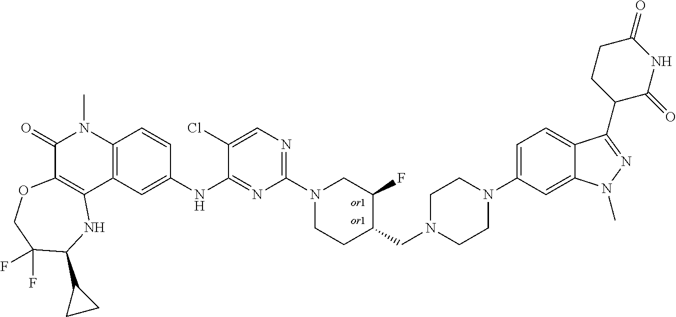 [SMILES: Cn1nc(C2CCC(=O)NC2=O)c2ccc(N3CCN(C[C@@H]4CCN(c5ncc(Cl)c(Nc6ccc7c(c6)c6c(c(=O)n7C)OCC(F)(F)[C@H](C7CC7)N6)n5)C[C@H]4F)CC3)cc21] |
| 579 | 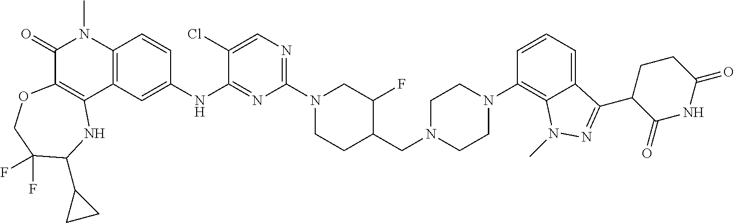 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)C(C7CC7)N6)n5)CC4F)CC3)c21] |
| 579a | 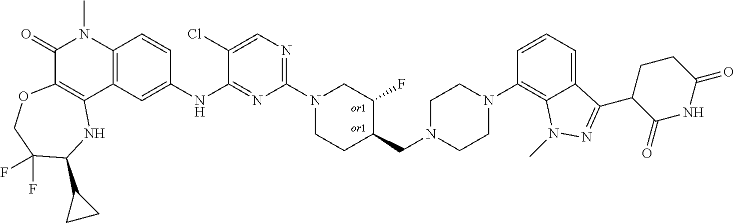 [SMILES: Cn1nc(C2CCC(=O)NC2=O)c2cccc(N3CCN(C[C@H]4CCN(c5ncc(Cl)c(Nc6ccc7c(c6)c6c(c(=O)n7C)OCC(F)(F)[C@H](C7CC7)N6)n5)C[C@@H]4F)CC3)c21] |
| 579b | 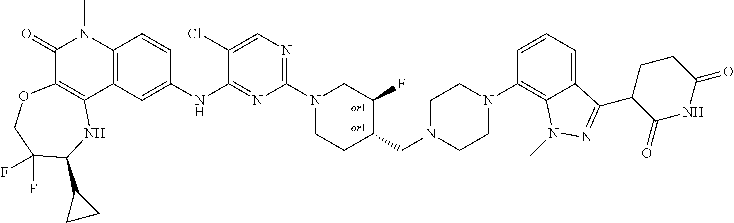 [SMILES: Cn1nc(C2CCC(=O)NC2=O)c2cccc(N3CCN(C[C@@H]4CCN(c5ncc(Cl)c(Nc6ccc7c(c6)c6c(c(=O)n7C)OCC(F)(F)[C@H](C7CC7)N6)n5)C[C@H]4F)CC3)c21] |
| 581 | 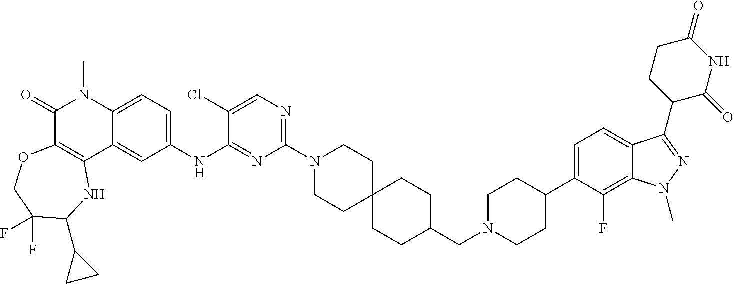 [SMILES: Cn1nc(C2CCC(=O)NC2=O)c2ccc(C3CCN(CC4CCC5(CC4)CCN(c4ncc(Cl)c(Nc6ccc7c(c6)c6c(c(=O)n7C)OCC(F)(F)C(C7CC7)N6)n4)CC5)CC3)c(F)c21] |
| 581a | 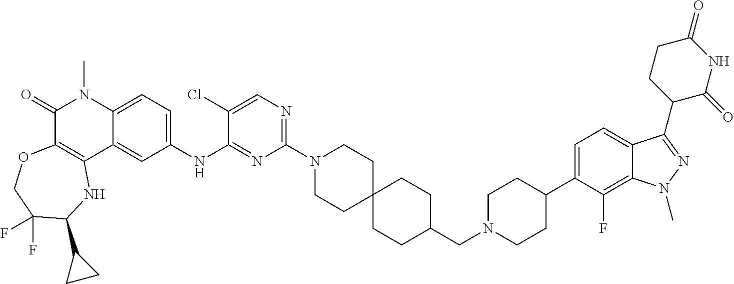 [SMILES: Cn1nc(C2CCC(=O)NC2=O)c2ccc(C3CCN(CC4CCC5(CC4)CCN(c4ncc(Cl)c(Nc6ccc7c(c6)c6c(c(=O)n7C)OCC(F)(F)[C@H](C7CC7)N6)n4)CC5)CC3)c(F)c21] |
| 582 | 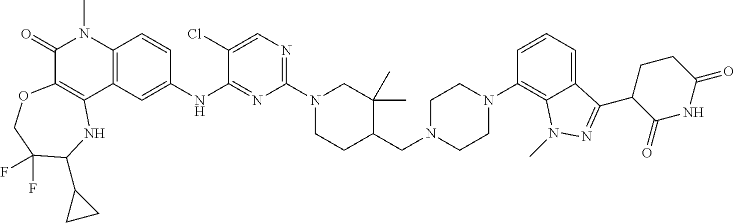 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)C(C7CC7)N6)n5)CC4(C)C)CC3)c21] |
| 582a | 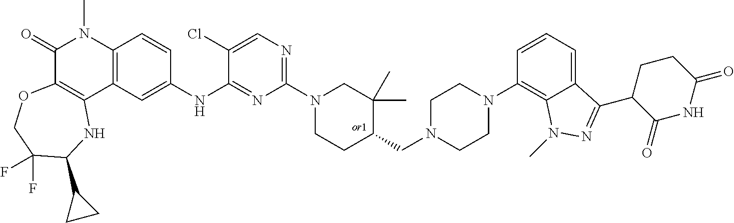 [SMILES: Cn1nc(C2CCC(=O)NC2=O)c2cccc(N3CCN(C[C@@H]4CCN(c5ncc(Cl)c(Nc6ccc7c(c6)c6c(c(=O)n7C)OCC(F)(F)[C@H](C7CC7)N6)n5)CC4(C)C)CC3)c21] |
| 582b | 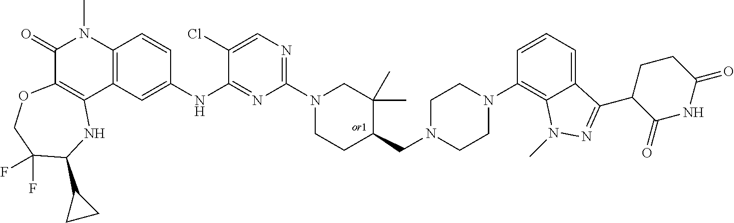 [SMILES: Cn1nc(C2CCC(=O)NC2=O)c2cccc(N3CCN(C[C@H]4CCN(c5ncc(Cl)c(Nc6ccc7c(c6)c6c(c(=O)n7C)OCC(F)(F)[C@H](C7CC7)N6)n5)CC4(C)C)CC3)c21] |

### Note:

In certain compounds of Table C1, one or more stereogenic centers are denoted with the “V3000 enhanced stereochemical notation” (see: support.collaborativedrug.com/hc/en-us/articles/360020872171-Advanced-S tereochemistry-Registration-Atropisomers-Mixtures-Unknowns-and-Non-Tetrahedral-Chirality, accessed on Dec. 23, 2022 and Accelrys Chemical Representation Guide, Accelrys Software Inc., 2014, each of which is incorporated by reference herein in its entirety). Using this stereochemical notation, certain stereogenic centers are denoted with “abs”, “&x”, or “orx”, wherein x is an integer (e.g., 1 or 2). For avoidance of doubt, the stereochemical notations in Table C1 have the following meaning:
(1) When a stereogenic center (e.g., a stereogenic carbon) is depicted with “flat” bonds (i.e., none of the chemical bonds at the stereogenic center is depicted with wedges or dashes) in a structural formula, each of said stereogenic centers can independently adopt the (R)- or (S)-configurations. For example, the structure

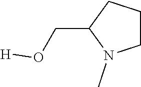

represents (S)-(1-methylpyrrolidin-2-yl)methanol, (R)-(1-methylpyrrolidin-2-yl)methanol, or a mixture thereof. As another non-limiting example, the structure

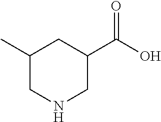

represents: (3S,5S)-5-methylpiperidine-3-carboxylic acid; (3R,5S)-5-methylpiperidine-3-carboxylic acid; (3S,5R)-5-methylpiperidine-3-carboxylic acid; (3R,5R)-5-methylpiperidine-3-carboxylic acid; or a mixture thereof.

When a stereogenic center or a plurality of stereogenic centers is depicted with wedges and dashes, the following notations are used:
(2) When a stereogenic center is denoted with “abs” or when a stereogenic center is not denoted with an enhanced stereochemical notation (e.g., “abs”, “&x”, or “orx”), the stereogenic center has the absolute configuration as depicted by the structural formula. For example, both of the structures

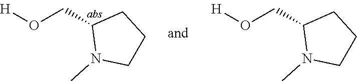

refer to (S)-(1-methylpyrrolidin-2-yl)methanol.
(3) When a stereogenic center is denoted with “orx” in a structural formula, the stereogenic center has been resolved but the configuration at the stereogenic center has not been determined. For example, the structure

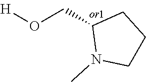

refers to one stereoisomer selected from the group consisting of (S)-(1-methylpyrrolidin-2-yl)methanol and (R)-(1-methylpyrrolidin-2-yl)methanol.
(4) When two or more stereogenic centers are denoted with “orx” in a structural formula, each of these stereogenic centers has been resolved but the configurations at said stereogenic centers have not been determined. Specifically:
   a. For any pair of stereogenic centers denoted with “orx” in a structural formula, when the numerical parts in the notation are different (e.g., two stereogenic centers denoted with “or1” and “or2” respectively), each stereogenic center is independently defined according to (3) (vide supra). For example, the structure refers to one stereoisomer selected from the group consisting of:

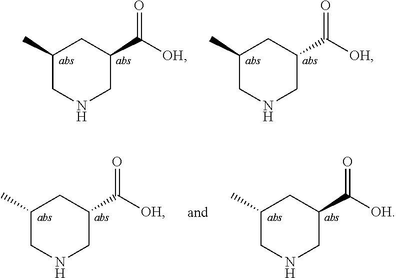

b. For any pair of stereogenic centers denoted with “orx” in a structural formula, when the numerical part in the notation is identical (e.g., two stereogenic centers each denoted with “or1”), the structural formula refers to one stereoisomer having the relative stereochemistry at these stereogenic centers as depicted in the structural formula, but the absolute configurations of these stereogenic centers have not been determined. For example, the structure

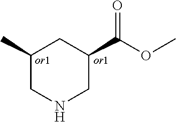

refers to one of the two “syn” stereoisomers:

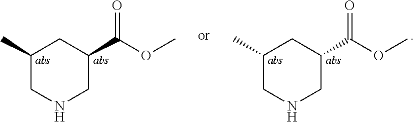

As another example, the structure

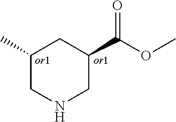

refers to one of the “anti” stereoisomers:

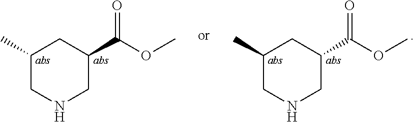

(5) When two or more stereogenic centers are denoted with “&x” in a structural formula, the structural formula refers to a mixture of stereoisomers that differ in the configuration at said stereogenic centers. Specifically:
   a. For any pair of stereogenic centers denoted with “&x” in a structural formula, when the numerical parts in the notation are different (e.g., two stereogenic centers denoted with “&1” and “&2” respectively), the structural formula refers to a mixture of stereoisomers at these two stereogenic centers, wherein the configuration at each stereogenic center can vary independently of one another. For example, the structure

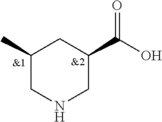

refers to a mixture of four stereoisomers:

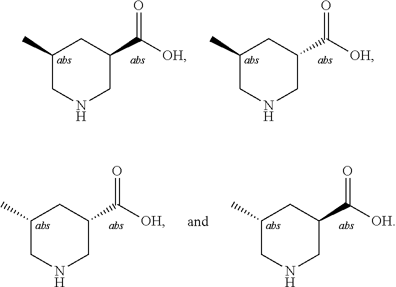

b. For any pair of stereogenic centers denoted with “&x” in a structural formula, when the numerical part in the notation is identical (e.g., two stereogenic centers each denoted with “&1”), the structural formula refers to a mixture of stereoisomers at these two or more stereogenic centers, wherein the relative configurations are as depicted in the structural formula. For example, the structure

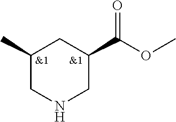

refers to a mixture of “syn” stereoisomers:

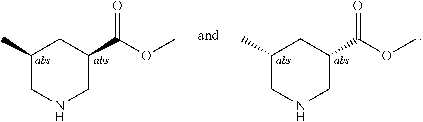

As another example, the structure

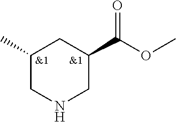

refers to a mixture of “anti” stereoisomers:

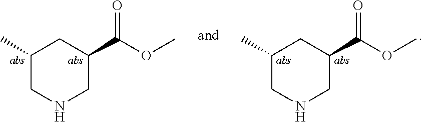

For example, for certain compounds in Table C1, when a stereogenic center is denoted with “or1” in a structural formula, the stereogenic center has been resolved but the configuration at the stereogenic center has not been determined. For example,

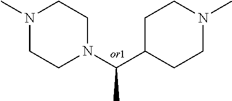

represents (R)-1-methyl-4-(1-(1-methylpiperidin-4-yl)ethyl)piperazine or (S)-1-methyl-4-(1-(1-methylpiperidin-4-yl)ethyl)piperazine.

Exemplary compounds of Formula (I-aa) (e.g., Formula (I-a)) include compounds: 101, 101a, 102,102a, 130,130a, 132,132a, 133,133a, 186,186a, 214, 214a, 214b, 379, 379a, 379b, 380, 380a, 380b, 381, 381a, 381b, 382, 382a, 383, 383a, 385, 385a, 386, 386a, 388, 388a, 391, 391a, 392, 392a, 393, 393a, 394, 394a, 395, 395a, 397, 397a, 398, 398a, 400, 400a, 403, 403a, 404, 404a, 405, 405a, 410, 410a, 411, 411a, 412, 412a, 414, 414a, 415, 415a, 416, 416a, 419, 419a, 420, 420a, 421, 421a, 422, 422a, 425, 425a, 426, 426a, 426b, 427, 427a, 428, 428a, 429, 429a, 430, 430a, 431, 431a, 432, 432a, 434, 434a, 435, 435a, 438, 438a, 440, 440a, 442, 442a, 442b, 443, 443a, 443b, 444, 444a, 445, 445a, 446, 446a, 450, 450a, 452, 452a, 453, 453a, 458, 458a, 461, 461a, 462, 462a, 463, 463a, 467, 467a, 469, 469a, 470, 470a, 472, 472a, 473, 473a, 474, 474a, 475, 475a, 475b, 476, 476a, 476b, 477, 477a, 479, 479a, 480, 480a, 481, 481a, 481b, 482, 482a, 482b, 483, 483a, 485, 485a, 486, 486a, 486b, 486c, 486d, 487, 487a, 487b, 487c, 487d, 488, 488a, 489, 489a, 491, 491a, 491b, 493, 493a, 494, 494a, 495, 495a, 496, 496a, 496b, 497, 497a, 498, 498a, 499, 499a, 504, 504a, 505, 505a, 506, 506a, 506b, 507, 507a, 509, 509a, 510, 510a, 513, 513a, 514, 514a, 516, 516a, 521, 521a, 522, 522a, 522b, 523, 523a, 523b, 525, 525a, 525b, 525c, 525d, 526, 526a, 527, 527a, 527b, 528, 528a, 529, 529a, 529b, 529c, 530, 530a, 531, 531a, 532, 532a, 533, 533a, 537, 537a, 538, 538a, 538b, 540, 540a, 541, 541a, 548, 548a, 548b, 550, 550a, 552, 552a, 553, 553a, 554, 554a, 555, 555a, 556, 556a, 557, 557a, 558, 558a, 561, 561a, 563, 563a, 563b, 564, 564a, 565, 565a, 568, 568a, 568b, 569, 569a, 572, 572a, 573, 573a, 574, 574a, 576, 576a, 577, 577a, 578, 578a, 578b, 579, 579a, 579b, 581, 581a, 582, 582a, and 582b, as depicted in Table C1, or pharmaceutically acceptable salts thereof.

Exemplary compounds of Formula (I-aa-1) (e.g., Formula (I-a-1)) include compounds: 101, 101a, 102, 102a, 130, 130a, 132, 132a, 133, 133a, 214, 214a, 214b, 379, 379a, 379b, 380, 380a, 380b, 381, 381a, 381b, 382, 382a, 383, 383a, 385, 385a, 386, 386a, 398, 398a, 400, 400a, 403, 403a, 404, 404a, 405, 405a, 412, 412a, 421, 421a, 422, 422a, 426, 426a, 426b, 442, 442a, 442b, 443, 443a, 443b, 452, 452a, 453, 453a, 461, 461a, 462, 462a, 463, 463a, 470, 470a, 472, 472a, 474, 474a, 475, 475a, 475b, 476, 476a, 476b, 479, 479a, 480, 480a, 485, 485a, 486, 486a, 486b, 486c, 486d, 487, 487a, 487b, 487c, 487d, 489, 489a, 491, 491a, 491b, 493, 493a, 495, 495a, 496, 496a, 496b, 497, 497a, 499, 499a, 504, 504a, 505, 505a, 506, 506a, 506b, 507, 507a, 510, 510a, 513, 513a, 514, 514a, 521, 521a, 522, 522a, 522b, 523, 523a, 523b, 525, 525a, 525b, 525c, 525d, 526, 526a, 527, 527a, 527b, 528, 528a, 529, 529a, 529b, 529c, 531, 531a, 533, 533a, 538, 538a, 538b, 548, 548a, 548b, 550, 550a, 554, 554a, 558, 558a, 561, 561a, 563, 563a, 563b, 564, 564a, 568, 568a, 568b, 569, 569a, 572, 572a, 574, 574a, 578, 578a, 578b, 579, 579a, 579b, 582, 582a, and 582b, as depicted in Table C1, or pharmaceutically acceptable salts thereof.

Exemplary compounds of Formula (I-aa-2) (e.g., Formula (I-a-2)) include compounds: 101, 101a, 102, 102a, 130, 130a, 132, 132a, 133, 133a, 214, 214a, 214b, 379, 379a, 379b, 380, 380a, 380b, 381, 381a, 381b, 382, 382a, 383, 383a, 385, 385a, 386, 386a, 398, 398a, 400, 400a, 403, 403a, 404, 404a, 405, 405a, 412, 412a, 421, 421a, 426, 426a, 426b, 442, 442a, 442b, 443, 443a, 443b, 452, 452a, 453, 453a, 461, 461a, 462, 462a, 463, 463a, 470, 470a, 472, 472a, 474, 474a, 475, 475a, 475b, 476, 476a, 476b, 479, 479a, 480, 480a, 485, 485a, 486, 486a, 486b, 486c, 486d, 487, 487a, 487b, 487c, 487d, 489, 489a, 491, 491a, 491b, 493, 493a, 495, 495a, 496, 496a, 496b, 497, 497a, 499, 499a, 504, 504a, 505, 505a, 506, 506a, 506b, 507, 507a, 510, 510a, 513, 513a, 514, 514a, 521, 521a, 522, 522a, 522b, 523, 523a, 523b, 525, 525a, 525b, 525c, 525d, 526, 526a, 531, 531a, 533, 533a, 538, 538a, 538b, 550, 550a, 554, 554a, 558, 558a, 561, 561a, 563, 563a, 563b, 564, 564a, 572, 572a, 578, 578a, 578b, 579, 579a, 579b, 582, 582a, and 582b, as depicted in Table C1, or pharmaceutically acceptable salts thereof.

Exemplary compounds of Formula (I-aa-3) (e.g., Formula (I-a-3)) include compounds: 186, 186a, 395, 395a, 416, 416a, 419, 419a, 434, 434a, 435, 435a, 438, 438a, 440, 440a, 444, 444a, 445, 445a, 446, 446a, 450, 450a, 458, 458a, 467, 467a, 469, 469a, 473, 473a, 477, 477a, 481, 481a, 481b, 482, 482a, 482b, 483, 483a, 488, 488a, 494, 494a, 498, 498a, 509, 509a, 516, 516a, 530, 530a, 555, 555a, 556, 556a, 557, 557a, 565, 565a, 573, 573a, 576, 576a, 577, 577a, and 581a, as depicted in Table C1, or pharmaceutically acceptable salts thereof.

Exemplary compounds of Formula (I-aa-4) (e.g., Formula (I-a-4)) include compounds: 416, 416a, 440, 440a, 458, 458a, 469, 469a, 473, 473a, 494, 494a, 565, 565a, 576, 576a, 577, 577a, 581, and 581a, as depicted in Table C1, or pharmaceutically acceptable salts thereof.

Exemplary compounds of Formula (I-aa-5) (e.g., Formula (I-a-5)) include compounds: 397, 397a, 420, 420a, 425, 425a, 432, 432a, 532, 532a, 537, 537a, 540, 540a, 541, and 541a, as depicted in Table C1, or pharmaceutically acceptable salts thereof.

Exemplary compounds of Formula (I-aa-6) (e.g., Formula (I-a-6)) include compounds: 388, 388a, 410, 410a, 411, 411a, 428, 428a, 429, 429a, 430, 430a, 431, 431a, 552, 552a, 553, and 553a, as depicted in Table C1, or pharmaceutically acceptable salts thereof.

Exemplary compounds of Formula (I-bb) (e.g., Formula (I-b)) include compounds: 389, 389a, 406, 406a, 407, 407a, 408, 408a, 409, 409a, 423, 423a, 424, 424a, 447, 447a, 449, 449a, 454, 454a, 455, 455a, 456, 456a, 457, 457a, 464, 464a, 464b, 464c, 464d, 465, 465a, 465b, 465c, 465d, 471, 471a, 492, 492a, 501, 501a, 502, 502a, 503, 503a, 508, 508a, 518, 518a, 520, 520a, 524, 524a, 534, 534a, 534b, 535, and 535a, as depicted in Table C1, or pharmaceutically acceptable salts thereof.

Exemplary compounds of Formula (I-bb-1) (e.g., Formula (I-b-1)) include compounds: 406, 406a, 407, 407a, 408, 408a, 409, 409a, 423, 423a, 424, 424a, 447, 447a, 449, 449a, 454, 454a, 455, 455a, 456, 456a, 457, 457a, 464, 464a, 464b, 464c, 464d, 465, 465a, 465b, 465c, 465d, 471, 471a, 492, 492a, 501, 501a, 502, 502a, 508, 508a, 518, 518a, 520, 520a, 524, 524a, 534, 534a, 534b, 535, and 535a, as depicted in Table C1, or pharmaceutically acceptable salts thereof.

Exemplary compounds of Formula (I-bb-2) (e.g., Formula (I-b-2)) include compounds: 406, 406a, 407, 407a, 447, 447a, 449, 449a, 454, 454a, 455, 455a, 464, 464a, 464b, 464c, 464d, 465, 465a, 465b, 465c, 465d, 471, 471a, 492, 492a, 501, 501a, 502, 502a, 534, 534a, 534b, 535, and 535a, as depicted in Table C1, or pharmaceutically acceptable salts thereof.

In some embodiments, the compounds of Formula (I) are selected from the group consisting of the compounds in Table C2, or pharmaceutically acceptable salts thereof.

TABLEC2

| Compound | |
|---|---|
| No. | CompoundName |
| 101 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-1-methyl-indazol-3-yl]piperidine-2,6-dione |
| 101a | 3-(7-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-1-methyl-1H-indazol-3-yl)piperidine-2,6-dione |
| 102 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-1-methyl-indazol-3-yl]piperidine-2,6-dione |
| 102a | 3-(6-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-1-methyl-1H-indazol-3-yl)piperidine-2,6-dione |
| 130 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazin-1- |
| | yl]-1-methyl-indazol-3-yl]piperidine-2,6-dione |
| 130a | 3-(7-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperazin-1-yl)-1-methyl-1H-indazol-3-yl)piperidine-2,6-dione |
| 132 | 3-[7-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-4-piperidyl]- |
| | 1-methyl-indazol-3-yl]piperidine-2,6-dione |
| 132a | 3-(7-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperidin-4-yl)-1-methyl-1H-indazol-3-yl)piperidine-2,6-dione |
| 133 | 3-[7-[1-[[1-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-4-piperidyl]methyl]-4- |
| | piperidyl]-1-methyl-indazol-3-yl]piperidine-2,6-dione |
| 133a | 3-(7-(1-((1-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)piperidin-4- |
| | yl)methyl)piperidin-4-yl)-1-methyl-1H-indazol-3-yl)piperidine-2,6-dione |
| 170 | 1-[7-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-1-methyl-indazol-3-yl]hexahydropyrimidine-2,4-dione |
| 170a | (S)-1-(7-(4-((4-(5-chloro-4-((2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-1-methyl-1H-indazol-3-yl)dihydropyrimidine-2,4(1H,3H)-dione |
| 186 | 3-[7-[7-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-2- |
| | azaspiro[3.5]nonan-2-yl]-1-methyl-indazol-3-yl]piperidine-2,6-dione |
| 186a | 3-(7-(7-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)-2-azaspiro[3.5]nonan-2-yl)-1-methyl-1H-indazol-3-yl)piperidine-2,6-dione |
| 194 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]oxy]-1-piperidyl]-1- |
| | methyl-indazol-3-yl]piperidine-2,6-dione |
| 194a | 3-(7-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)oxy)piperidin-1-yl)-1-methyl-1H-indazol-3-yl)piperidine-2,6-dione |
| 214 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-methyl-piperazin-1- |
| | yl]methyl]-1-piperidyl]-1-methyl-indazol-3-yl]piperidine-2,6-dione |
| 214a | 3-(7-(4-(((R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2-methylpiperazin-1- |
| | yl)methyl)piperidin-1-yl)-1-methyl-1H-indazol-3-yl)piperidine-2,6-dione |
| 214b | 3-(7-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2-methylpiperazin-1- |
| | yl)methyl)piperidin-1-yl)-1-methyl-1H-indazol-3-yl)piperidine-2,6-dione |
| 374 | 4-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-2-(2,6-dioxo-3-piperidyl)isoindoline-1,3-dione |
| 374a | 4-(4-((4-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)piperazin-1-yl)methyl)piperidin- |
| | 1-yl)-2-(2,6-dioxopiperidin-3-yl)isoindoline-1,3-dione |
| 375 | 4-[4-[[4-[5-chloro-4-[(2-cyclopropyl-3,3-difluoro-7-isopropyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-2-(2,6-dioxo-3-piperidyl)isoindoline-1,3-dione |
| 375a | 4-(4-((4-(5-chloro-4-(((S)-2-cyclopropyl-3,3-difluoro-7-isopropyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-2-(2,6-dioxopiperidin-3-yl)isoindoline-1,3-dione |
| 379 | 3-[6-[4-[1-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]ethyl]-1- |
| | piperidyl]-1-methyl-indazol-3-yl]piperidine-2,6-dione |
| 379a | 3-(6-(4-((S*)-1-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)ethyl)piperidin-1-yl)-1-methyl-1H-indazol-3-yl)piperidine-2,6-dione |
| 379b | 3-(6-(4-((R*)-1-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)ethyl)piperidin-1-yl)-1-methyl-1H-indazol-3-yl)piperidine-2,6-dione |
| 380 | 3-[7-[4-[1-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]ethyl]-1- |
| | piperidyl]-1-methyl-indazol-3-yl]piperidine-2,6-dione |
| 380a | 3-(7-(4-((S*)-1-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)ethyl)piperidin-1-yl)-1-methyl-1H-indazol-3-yl)piperidine-2,6-dione |
| 380b | 3-(7-(4-((R*)-1-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)ethyl)piperidin-1-yl)-1-methyl-1H-indazol-3-yl)piperidine-2,6-dione |
| 381 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-methyl-piperazin-1- |
| | yl]methyl]-1-piperidyl]-1-methyl-indazol-3-yl]piperidine-2,6-dione |
| 381a | 3-(6-(4-(((R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2-methylpiperazin-1- |
| | yl)methyl)piperidin-1-yl)-1-methyl-1H-indazol-3-yl)piperidine-2,6-dione |
| 381b | 3-(6-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2-methylpiperazin-1- |
| | yl)methyl)piperidin-1-yl)-1-methyl-1H-indazol-3-yl)piperidine-2,6-dione |
| 382 | 3-[6-[4-[[4-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]piperazin-1-yl]methyl]- |
| | 1-piperidyl]-1-methyl-indazol-3-yl]piperidine-2,6-dione |
| 382a | 3-(6-(4-((4-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)piperazin-1- |
| | yl)methyl)piperidin-1-yl)-1-methyl-1H-indazol-3-yl)piperidine-2,6-dione |
| 383 | 3-[7-[4-[[4-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]piperazin-1-yl]methyl]- |
| | 1-piperidyl]-1-methyl-indazol-3-yl]piperidine-2,6-dione |
| 383a | 3-(7-(4-((4-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)piperazin-1- |
| | yl)methyl)piperidin-1-yl)-1-methyl-1H-indazol-3-yl)piperidine-2,6-dione |
| 384 | 3-[7-[4-[[4-[5-bromo-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-1-methyl-indazol-3-yl]piperidine-2,6-dione |
| 384a | 3-(7-(4-((4-(5-bromo-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-1-methyl-1H-indazol-3-yl)piperidine-2,6-dione |
| 385 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]piperazin-1-yl]methyl]-1-piperidyl]-1- |
| | methyl-indazol-3-yl]piperidine-2,6-dione |
| 385a | 3-(6-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperazin-1- |
| | yl)methyl)piperidin-1-yl)-1-methyl-1H-indazol-3-yl)piperidine-2,6-dione |
| 386 | 3-[7-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]piperazin-1-yl]methyl]-1-piperidyl]-1- |
| | methyl-indazol-3-yl]piperidine-2,6-dione |
| 386a | 3-(7-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperazin-1- |
| | yl)methyl)piperidin-1-yl)-1-methyl-1H-indazol-3-yl)piperidine-2,6-dione |
| 387 | 3-[3-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazine-1- |
| | carbonyl]phenyl]piperidine-2,6-dione |
| 387a | 3-(3-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperazine-1-carbonyl)phenyl)piperidine-2,6-dione |
| 388 | 3-[7-[3-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]azetidin-1-yl]-1- |
| | methyl-indazol-3-yl]piperidine-2,6-dione |
| 388a | 3-(7-(3-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)azetidin-1-yl)-1-methyl-1H-indazol-3-yl)piperidine-2,6-dione |
| 389 | 3-[6-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-piperidyl]methylamino]-1- |
| | methyl-indazol-3-yl]piperidine-2,6-dione |
| 389a | 3-(6-((((R)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-3- |
| | yl)methyl)amino)-1-methyl-1H-indazol-3-yl)piperidine-2,6-dione |
| 390 | 3-[6-[4-[[[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2- |
| | yl]aminomethyl]cyclohexyl]amino]-1-methyl-indazol-3-yl]piperidine-2,6-dione |
| 390a | 3-(6-(((1s,4r)-4-(((5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2- |
| | yl)amino)methyl)cyclohexyl)amino)-1-methyl-1H-indazol-3-yl)piperidine-2,6-dione |
| 391 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]piperidine- |
| | 1-carbonyl]-1-methyl-indazol-3-yl]piperidine-2,6-dione |
| 391a | 3-(6-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidine-1-carbonyl)-1-methyl-1H-indazol-3-yl)piperidine-2,6-dione |
| 392 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazine-1- |
| | carbonyl]-1-methyl-indazol-3-yl]piperidine-2,6-dione |
| 392a | 3-(6-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperazine-1-carbonyl)-1-methyl-1H-indazol-3-yl)piperidine-2,6-dione |
| 393 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]piperidine- |
| | 1-carbonyl]-1-methyl-indazol-3-yl]piperidine-2,6-dione |
| 393a | 3-(7-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidine-1-carbonyl)-1-methyl-1H-indazol-3-yl)piperidine-2,6-dione |
| 394 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazine-1- |
| | carbonyl]-1-methyl-indazol-3-yl]piperidine-2,6-dione |
| 394a | 3-(7-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperazine-1-carbonyl)-1-methyl-1H-indazol-3-yl)piperidine-2,6-dione |
| 395 | 3-[7-[4-[[8-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,8-diazabicyclo[3.2.1]octan-3- |
| | yl]methyl]-1-piperidyl]-1-methyl-indazol-3-yl]piperidine-2,6-dione |
| 395a | 3-(7-(4-((8-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3,8- |
| | diazabicyclo[3.2.1]octan-3-yl)methyl)piperidin-1-yl)-1-methyl-1H-indazol-3-yl)piperidine- |
| | 2,6-dione |
| 396 | 3-[6-[8-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,8-diazaspiro[3.5]nonan-2-yl]- |
| | 1-methyl-indazol-3-yl]piperidine-2,6-dione |
| 396a | 3-(6-(6-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2,6-diazaspiro[3.5]nonan-2-yl)- |
| | 1-methyl-1H-indazol-3-yl)piperidine-2,6-dione |
| 397 | 3-[7-[4-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperidine-4-carbonyl]piperazin- |
| | 1-yl]-1-methyl-indazol-3-yl]piperidine-2,6-dione |
| 397a | 3-(7-(4-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidine-4- |
| | carbonyl)piperazin-1-yl)-1-methyl-1H-indazol-3-yl)piperidine-2,6-dione |
| 398 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,6-dimethyl-piperazin-1- |
| | yl]methyl]-1-piperidyl]-1-methyl-indazol-3-yl]piperidine-2,6-dione |
| 398a | 3-(7-(4-(((2S,6R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,6- |
| | dimethylpiperazin-1-yl)methyl)piperidin-1-yl)-1-methyl-1H-indazol-3-yl)piperidine-2,6- |
| | dione |
| 399 | 3-[7-[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,8-diazaspiro[5.5]undecan-8- |
| | yl]-1-methyl-indazol-3-yl]piperidine-2,6-dione |
| 399a | 3-(7-(8-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2,8-diazaspiro[5.5]undecan-2- |
| | yl)-1-methyl-1H-indazol-3-yl)piperidine-2,6-dione |
| 400 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazin-1- |
| | yl]-1-methyl-indazol-3-yl]piperidine-2,6-dione |
| 400a | 3-(6-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperazin-1-yl)-1-methyl-1H-indazol-3-yl)piperidine-2,6-dione |
| 401 | 3-[6-[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,8-diazaspiro[5.5]undecan-8- |
| | yl]-1-methyl-indazol-3-yl]piperidine-2,6-dione |
| 401a | 3-(6-(8-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2,8-diazaspiro[5.5]undecan-2- |
| | yl)-1-methyl-1H-indazol-3-yl)piperidine-2,6-dione |
| 402 | 3-[7-[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,7-diazaspiro[4.5]decan-2-yl]- |
| | 1-methyl-indazol-3-yl]piperidine-2,6-dione |
| 402a | 3-(7-(7-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2,7-diazaspiro[4.5]decan-2-yl)- |
| | 1-methyl-1H-indazol-3-yl)piperidine-2,6-dione |
| 403 | 3-[7-[4-[[4-[5-chloro-4-[(2-cyclopropyl-3,3-difluoro-7-isopropyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-1-methyl-indazol-3-yl]piperidine-2,6-dione |
| 403a | 3-(7-(4-((4-(5-chloro-4-(((S)-2-cyclopropyl-3,3-difluoro-7-isopropyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-1-methyl-1H-indazol-3-yl)piperidine-2,6-dione |
| 404 | 3-[6-[4-[[4-[5-chloro-4-[(2-cyclopropyl-3,3-difluoro-7-isopropyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-1-methyl-indazol-3-yl]piperidine-2,6-dione |
| 404a | 3-(6-(4-((4-(5-chloro-4-(((S)-2-cyclopropyl-3,3-difluoro-7-isopropyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-1-methyl-1H-indazol-3-yl)piperidine-2,6-dione |
| 405 | 3-[6-[4-[1-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]-1-methyl-ethyl]- |
| | 1-piperidyl]-1-methyl-indazol-3-yl]piperidine-2,6-dione |
| 405a | 3-(6-(4-(2-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1-yl)propan- |
| | 2-yl)piperidin-1-yl)-1-methyl-1H-indazol-3-yl)piperidine-2,6-dione |
| 406 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7- |
| | yl]amino]-1-methyl-indazol-3-yl]piperidine-2,6-dione |
| 406a | 3-(6-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[3.5]nonan-7- |
| | yl)amino)-1-methyl-1H-indazol-3-yl)piperidine-2,6-dione |
| 407 | 3-[7-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7- |
| | yl]amino]-1-methyl-indazol-3-yl]piperidine-2,6-dione |
| 407a | 3-(7-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[3.5]nonan-7- |
| | yl)amino)-1-methyl-1H-indazol-3-yl)piperidine-2,6-dione |
| 408 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[4.5]decan-8- |
| | yl]amino]-1-methyl-indazol-3-yl]piperidine-2,6-dione |
| 408a | 3-(6-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[4.5]decan-8- |
| | yl)amino)-1-methyl-1H-indazol-3-yl)piperidine-2,6-dione |
| 409 | 3-[7-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[4.5]decan-8- |
| | yl]amino]-1-methyl-indazol-3-yl]piperidine-2,6-dione |
| 409a | 3-(7-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[4.5]decan-8- |
| | yl)amino)-1-methyl-1H-indazol-3-yl)piperidine-2,6-dione |
| 410 | 3-[7-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]-1-piperidyl]-1- |
| | methyl-indazol-3-yl]piperidine-2,6-dione |
| 410a | 3-(7-(4-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)piperidin-1-yl)-1-methyl-1H-indazol-3-yl)piperidine-2,6-dione |
| 411 | 3-[6-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]-1-piperidyl]-1- |
| | methyl-indazol-3-yl]piperidine-2,6-dione |
| 411a | 3-(6-(4-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)piperidin-1-yl)-1-methyl-1H-indazol-3-yl)piperidine-2,6-dione |
| 412 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-4-piperidyl]- |
| | 1-methyl-indazol-3-yl]piperidine-2,6-dione |
| 412a | 3-(6-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperidin-4-yl)-1-methyl-1H-indazol-3-yl)piperidine-2,6-dione |
| 413 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-1,4-diazepan-1-yl]methyl]-1- |
| | piperidyl]-1-methyl-indazol-3-yl]piperidine-2,6-dione |
| 413a | 3-(7-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-1,4-diazepan-1- |
| | yl)methyl)piperidin-1-yl)-1-methyl-1H-indazol-3-yl)piperidine-2,6-dione |
| 414 | 3-[6-[4-[2-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]ethyl]-1- |
| | piperidyl]-1-methyl-indazol-3-yl]piperidine-2,6-dione |
| 414a | 3-(6-(4-(2-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)ethyl)piperidin-1-yl)-1-methyl-1H-indazol-3-yl)piperidine-2,6-dione |
| 415 | 3-[7-[4-[2-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-ylethyl]-1- |
| | piperidyl]-1-methyl-indazol-3-yl]piperidine-2,6-dione |
| 415a | 3-(7-(4-(2-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)ethyl)piperidin-1-yl)-1-methyl-1H-indazol-3-yl)piperidine-2,6-dione |
| 416 | 3-[6-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,9-diazaspiro[5.5]undecan-9- |
| | yl]methyl]-1-piperidyl]-1-methyl-indazol-3-yl]piperidine-2,6-dione |
| 416a | 3-(6-(4-((9-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3,9- |
| | diazaspiro[5.5]undecan-3-yl)methyl)piperidin-1-yl)-1-methyl-1H-indazol-3-yl)piperidine- |
| | 2,6-dione |
| 417 | 3-[6-[5-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,3a,4,6,7,7a-hexahydro-1H- |
| | pyrrolo[3,4-c]pyridin-2-yl]-1-methyl-indazol-3-yl]piperidine-2,6-dione |
| 417a | 3-[6-[5-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-3,3a,4,6,7,7a-hexahydro-1H- |
| | pyrrolo[3,4-c]pyridin-2-yl]-1-methyl-indazol-3-yl]piperidine-2,6-dione |
| 418 | 3-[7-[5-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,3a,4,6,7,7a-hexahydro-1H- |
| | pyrrolo[3,4-c]pyridin-2-yl]-1-methyl-indazol-3-yl]piperidine-2,6-dione |
| 418a | 3-(7-(5-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)octahydro-2H-pyrrolo[3,4- |
| | c]pyridin-2-yl)-1-methyl-1H-indazol-3-yl)piperidine-2,6-dione |
| 419 | 3-[7-[4-[[9-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-oxa-7,9- |
| | diazabicyclo[3.3.1]nonan-7-yl]methyl]-1-piperidyl]-1-methyl-indazol-3-yl]piperidine-2,6- |
| | dione |
| 419a | 3-(7-(4-((9-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-oxa-7,9- |
| | diazabicyclo[3.3.1]nonan-7-yl)methyl)piperidin-1-yl)-1-methyl-1H-indazol-3-yl)piperidine- |
| | 2,6-dione |
| 420 | 3-[6-[4-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperidine-4-carbonyl]piperazin- |
| | 1-yl]-1-methyl-indazol-3-yl]piperidine-2,6-dione |
| 420a | 3-(6-(4-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidine-4- |
| | carbonyl)piperazin-1-yl)-1-methyl-1H-indazol-3-yl)piperidine-2,6-dione |
| 421 | 3-[7-[4-[1-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]-1-methyl-ethyl]- |
| | 1-piperidyl]-1-methyl-indazol-3-yl]piperidine-2,6-dione |
| 421a | 3-(7-(4-(2-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1-yl)propan- |
| | 2-yl)piperidin-1-yl)-1-methyl-1H-indazol-3-yl)piperidine-2,6-dione |
| 422 | 3-[7-[3-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]azetidin-1- |
| | yl]-1-methyl-indazol-3-yl]piperidine-2,6-dione |
| 422a | 3-(7-(3-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)azetidin-1-yl)-1-methyl-1H-indazol-3-yl)piperidine-2,6-dione |
| 423 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-1,3,3a,4,5,6,7,7a- |
| | octahydroisoindol-5-yl]amino]-1-methyl-indazol-3-yl]piperidine-2,6-dione |
| 423a | 3-(6-((2-(5-chloro-4-(((S)-2-cyclopropyl-3,3-difluoro-7-methyl-6-oxo-1,2,3,4,6,7-hexahydro- |
| | [1,4]-oxazepino[2,3-c]-quinolin-10-yl)amino)pyrimidin-2-yl)octahydro-1H-isoindol-5- |
| | yl)amino)-1-methyl-1H-indazol-3-yl)piperidine-2,6-dione |
| 424 | 3-[7-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-1,3,3a,4,5,6,7,7a- |
| | octahydroisoindol-5-yl]amino]-1-methyl-indazol-3-yl]piperidine-2,6-dione |
| 424a | 3-(7-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)octahydro-1H-isoindol-5- |
| | yl)amino)-1-methyl-1H-indazol-3-yl)piperidine-2,6-dione |
| 425 | 3-[7-[4-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]piperidine-4-carbonyl]piperazin-1-yl]- |
| | 1-methyl-indazol-3-yl]piperidine-2,6-dione |
| 425a | 3-(7-(4-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperidine-4- |
| | carbonyl)piperazin-1-yl)-1-methyl-1H-indazol-3-yl)piperidine-2,6-dione |
| 426 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-(trifluoromethyl)piperazin-1- |
| | yl]methyl]-1-piperidyl]-1-methyl-indazol-3-yl]piperidine-2,6-dione |
| 426a | 3-(7-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2- |
| | (trifluoromethyl)piperazin-1-yl)methyl)piperidin-1-yl)-1-methyl-1H-indazol-3-yl)piperidine- |
| | 2,6-dione |
| 426b | 3-(7-(4-(((R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2- |
| | (trifluoromethyl)piperazin-1-yl)methyl)piperidin-1-yl)-1-methyl-1H-indazol-3-yl)piperidine- |
| | 2,6-dione |
| 427 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-1,4-diazepan-1-yl]methyl]-1- |
| | piperidyl]-1-methyl-indazol-3-yl]piperidine-2,6-dione |
| 427a | 3-(6-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-1,4-diazepan-1- |
| | yl)methyl)piperidin-1-yl)-1-methyl-1H-indazol-3-yl)piperidine-2,6-dione |
| 428 | 3-[6-[3-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]-1-piperidyl]-1- |
| | methyl-indazol-3-yl]piperidine-2,6-dione |
| 428a | 3-(6-(3-(4-(5-chloro-4-(((S)-2-cyclopropyl-3,3-difluoro-7-methyl-6-oxo-1,2,3,4,6,7- |
| | hexahydro-[1,4]-oxazepino[2,3-c]quinolin-10-yl)amino)pyrimidin-2-yl)piperazin-1- |
| | yl)piperidin-1-yl)-1-methyl-1H-indazol-3-yl)piperidine-2,6-dione |
| 429 | 3-[7-[3-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]-1-piperidyl]-1- |
| | methyl-indazol-3-yl]piperidine-2,6-dione |
| 429a | 3-(7-(3-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)piperidin-1-yl)-1-methyl-1H-indazol-3-yl)piperidine-2,6-dione |
| 430 | 3-[6-[3-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]pyrrolidin-1-yl]-1- |
| | methyl-indazol-3-yl]piperidine-2,6-dione |
| 430a | 3-(6-(3-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1-yl) |
| | pyrrolidin-1-yl)-1-methyl-1H-indazol-3-yl)piperidine-2,6-dione |
| 431 | 3-[7-[3-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]pyrrolidin-1-yl]-1- |
| | methyl-indazol-3-yl]piperidine-2,6-dione |
| 431a | 3-(7-(3-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)pyrrolidin-1-yl)-1-methyl-1H-indazol-3-yl)piperidine-2,6-dione |
| 432 | 3-[7-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazine-1-carbonyl]-1- |
| | piperidyl]-1-methyl-indazol-3-yl]piperidine-2,6-dione |
| 432a | 3-(7-(4-(4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazine-1- |
| | carbonyl)piperidin-1-yl)-1-methyl-1H-indazol-3-yl)piperidine-2,6-dione |
| 433 | 4-[3-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]azetidin-1- |
| | yl]-2-(2,6-dioxo-3-piperidyl)isoindoline-1,3-dione |
| 433a | 4-(3-((1-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)piperidin-4-yl)methyl)azetidin-1- |
| | yl)-2-(2,6-dioxopiperidin-3-yl)isoindoline-1,3-dione |
| 434 | 3-[7-[4-[[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-oxa-7,9- |
| | diazabicyclo[3.3.1]nonan-9-yl]methyl]-1-piperidyl]-1-methyl-indazol-3-yl]piperidine-2,6- |
| | dione |
| 434a | 3-(7-(4-((7-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-oxa-7,9- |
| | diazabicyclo[3.3.1]nonan-9-yl)methyl)piperidin-1-yl)-1-methyl-1H-indazol-3-yl)piperidine- |
| | 2,6-dione |
| 435 | 3-[7-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-azabicyclo[3.1.0]hexan-6- |
| | yl]methyl]piperazin-1-yl]-1-methyl-indazol-3-yl]piperidine-2,6-dione |
| 435a | 3-(7-(4-(((1R,5S,6R)-3-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | azabicyclo[3.1.0]hexan-6-yl)methyl)piperazin-1-yl)-1-methyl-1H-indazol-3-yl)piperidine- |
| | 2,6-dione |
| 436 | 3-[7-[4-[[4-[4,5-dichloro-6-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]-1-methyl-indazol-3-yl]piperidine-2,6-dione |
| 436a | 3-(7-(4-((4-(4,5-dichloro-6-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)-1-methyl-1H-indazol-3-yl)piperidine-2,6-dione |
| 437 | 4-[(2-cyclopropyl-3,3-difluoro-7-methyl-6-oxo-2,4-dihydro-1H-[1,4]oxazepino[2,3- |
| | c]quinolin-10-yl)amino]-2-[4-[[1-[3-(2,6-dioxo-3-piperidyl)-1-methyl-indazol-7-yl]-4- |
| | piperidyl]methyl]piperazin-1-yl]pyrimidine-5-carbonitrile |
| 437a | 4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-2-(4-((1-(3-(2,6-dioxopiperidin-3-yl)-1-methyl- |
| | 1H-indazol-7-yl)piperidin-4-yl)methyl)piperazin-1-yl)pyrimidine-5-carbonitrile |
| 438 | 3-[6-[4-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,6-diazaspiro[3.3]heptan-6- |
| | yl]methyl]-1-piperidyl]-1-methyl-indazol-3-yl]piperidine-2,6-dione |
| 438a | 3-(6-(4-((6-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,6- |
| | diazaspiro[3.3]heptan-2-yl)methyl)piperidin-1-yl)-1-methyl-1H-indazol-3-yl)piperidine-2,6- |
| | dione |
| 439 | 3-[6-[[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]methyl]-1-methyl-indazol-3-yl]piperidine-2,6-dione |
| 439a | 3-(6-((4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)methyl)-1-methyl-1H-indazol-3-yl)piperidine-2,6-dione |
| 440 | 3-[7-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,9-diazaspiro[5.5]undecan-9- |
| | yl]methyl]-1-piperidyl]-1-methyl-indazol-3-yl]piperidine-2,6-dione |
| 440a | 3-(7-(4-((9-(5-chloro-4-(((S)-2-cyclopropyl-3,3-difluoro-7-methyl-6-oxo-1,2,3,4,6,7- |
| | hexahydro[1,4]oxazepino[2,3-c]quinolin-10-yl)amino)pyrimidin-2-yl)-3,9- |
| | diazaspiro[5.5]undecan-3-yl)methyl)piperidin-1-yl)-1-methyl-1H-indazol-3-yl)piperidine- |
| | 2,6-dione |
| 441 | 4-chloro-6-[(2-cyclopropyl-3,3-difluoro-7-methyl-6-oxo-2,4-dihydro-1H-[1,4]oxazepino[2,3- |
| | c]quinolin-10-yl)amino]-2-[4-[[1-[3-(2,6-dioxo-3-piperidyl)-1-methyl-indazol-7-yl]-4- |
| | piperidyl]methyl]piperazin-1-yl]pyrimidine-5-carbonitrile |
| 441a | 4-chloro-6-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-2-(4-((1-(3-(2,6-dioxopiperidin-3-yl)-1-methyl- |
| | 1H-indazol-7-yl)piperidin-4-yl)methyl)piperazin-1-yl)pyrimidine-5-carbonitrile |
| 442 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3-methyl- |
| | piperazin-1-yl]-1-methyl-indazol-3-yl]piperidine-2,6-dione |
| 442a | 3-(7-((S)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3-methylpiperazin-1-yl)-1-methyl-1H-indazol-3-yl)piperidine-2,6-dione |
| 442b | 3-(7-((R)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3-methylpiperazin-1-yl)-1-methyl-1H-indazol-3-yl)piperidine-2,6-dione |
| 443 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3-methyl- |
| | piperazin-1-yl]-1-methyl-indazol-3-yl]piperidine-2,6-dione |
| 443a | 3-(6-((R)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3-methylpiperazin-1-yl)-1-methyl-1H-indazol-3-yl)piperidine-2,6-dione |
| 443b | 3-(6-((S)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3-methylpiperazin-1-yl)-1-methyl-1H-indazol-3-yl)piperidine-2,6-dione |
| 444 | 3-[7-[8-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-8- |
| | azabicyclo[3.2.1]octan-3-yl]-1-methyl-indazol-3-yl]piperidine-2,6-dione |
| 444a | 3-(7-((1r,5s)-8-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-8-azabicyclo[3.2.1]octan-3-yl)-1-methyl-1H-indazol-3-yl)piperidine-2,6-dione |
| 445 | 3-[6-[4-[[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,7-diazaspiro[3.5]nonan-2- |
| | yl]methyl]-1-piperidyl]-1-methyl-indazol-3-yl]piperidine-2,6-dione |
| 445a | 3-(6-(4-((7-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,7- |
| | diazaspiro[3.5]nonan-2-yl)methyl)piperidin-1-yl)-1-methyl-1H-indazol-3-yl)piperidine-2,6- |
| | dione |
| 446 | 3-[7-[4-[[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,7-diazaspiro[3.5]nonan-2- |
| | yl]methyl]-1-piperidyl]-1-methyl-indazol-3-yl]piperidine-2,6-dione |
| 446a | 3-(7-(4-((7-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,7- |
| | diazaspiro[3.5]nonan-2-yl)methyl)piperidin-1-yl)-1-methyl-1H-indazol-3-yl)piperidine-2,6- |
| | dione |
| 447 | 3-[7-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7-yl]oxy]- |
| | 1-methyl-indazol-3-yl]piperidine-2,6-dione |
| 447a | 3-(7-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[3.5]nonan-7-yl)oxy)- |
| | 1-methyl-1H-indazol-3-yl)piperidine-2,6-dione |
| 448 | 3-[7-[[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]-1- |
| | piperidyl]methyl]-1-methyl-indazol-3-yl]piperidine-2,6-dione |
| 448a | 3-(7-((4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)piperidin-1-yl)methyl)-1-methyl-1H-indazol-3-yl)piperidine-2,6-dione |
| 449 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7-yl]oxy]- |
| | 1-methyl-indazol-3-yl]piperidine-2,6-dione |
| 449a | 3-(6-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[3.5]nonan-7-yl)oxy)- |
| | 1-methyl-1H-indazol-3-yl)piperidine-2,6-dione |
| 450 | 3-[7-[4-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,6-diazaspiro[3.3]heptan-6- |
| | yl]methyl]-1-piperidyl]-1-methyl-indazol-3-yl]piperidine-2,6-dione |
| 450a | 3-(7-(4-((6-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,6- |
| | diazaspiro[3.3]heptan-2-yl)methyl)piperidin-1-yl)-1-methyl-1H-indazol-3-yl)piperidine-2,6- |
| | dione |
| 451 | 3-[7-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-(hydroxymethyl)piperazin-1- |
| | yl]methyl]-1-piperidyl]-1-methyl-indazol-3-yl]piperidine-2,6-dione |
| 451a | 3-(7-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2- |
| | (hydroxymethyl)piperazin-1-yl)methyl)piperidin-1-yl)-1-methyl-1H-indazol-3-yl)piperidine- |
| | 2,6-dione |
| 451b | 3-(7-(4-(((R)-4-(5-chloro-4-(((S)-2-cyclopropyl-3,3-difluoro-7-methyl-6-oxo-1,2,3,4,6,7- |
| | hexahydro-[1,4]-oxazepino[2,3-c]-quinolin-10-yl)amino)pyrimidin-2-yl)-2- |
| | (hydroxymethyl)piperazin-1-yl)methyl)piperidin-1-yl)-1-methyl-1H-indazol-3-yl)piperidine- |
| | 2,6-dione |
| 452 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-4-piperidyl]methyl]piperazin-1-yl]-1- |
| | methyl-indazol-3-yl]piperidine-2,6-dione |
| 452a | 3-(6-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperidin-4- |
| | yl)methyl)piperazin-1-yl)-1-methyl-1H-indazol-3-yl)piperidine-2,6-dione |
| 453 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-4-piperidyl]methyl]piperazin-1-yl]-1- |
| | methyl-indazol-3-yl]piperidine-2,6-dione |
| 453a | 3-(7-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperidin-4- |
| | yl)methyl)piperazin-1-yl)-1-methyl-1H-indazol-3-yl)piperidine-2,6-dione |
| 454 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7-yl]- |
| | methyl-amino]-1-methyl-indazol-3-yl]piperidine-2,6-dione |
| 454a | 3-(6-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[3.5]nonan-7- |
| | yl)(methyl)amino)-1-methyl-1H-indazol-3-yl)piperidine-2,6-dione |
| 455 | 3-[7-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7-yl]- |
| | methyl-amino]-1-methyl-indazol-3-yl]piperidine-2,6-dione |
| 455a | 3-(7-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[3.5]nonan-7- |
| | yl)(methyl)amino)-1-methyl-1H-indazol-3-yl)piperidine-2,6-dione |
| 456 | 3-[6-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]oxy]-1-methyl- |
| | indazol-3-yl]piperidine-2,6-dione |
| 456a | 3-(6-((1-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)piperidin-4-yl)oxy)-1-methyl-1H- |
| | indazol-3-yl)piperidine-2,6-dione |
| 457 | 3-[7-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]oxy]-1-methyl- |
| | indazol-3-yl]piperidine-2,6-dione |
| 457a | 3-(7-((1-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)piperidin-4-yl)oxy)-1-methyl-1H- |
| | indazol-3-yl)piperidine-2,6-dione |
| 458 | 3-[6-[4-[[3-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-3,9- |
| | diazaspiro[5.5]undecan-9-yl]methyl]-1-piperidyl]-1-methyl-indazol-3-yl]piperidine-2,6-dione |
| 458a | 3-(6-(4-((9-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)-3,9- |
| | diazaspiro[5.5]undecan-3-yl)methyl)piperidin-1-yl)-1-methyl-1H-indazol-3-yl)piperidine- |
| | 2,6-dione |
| 459 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-(hydroxymethyl)piperazin-1- |
| | yl]methyl]-1-piperidyl]-1-methyl-indazol-3-yl]piperidine-2,6-dione |
| 459a | 3-(6-(4-(((R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2- |
| | (hydroxymethyl)piperazin-1-yl)methyl)piperidin-1-yl)-1-methyl-1H-indazol-3-yl)piperidine- |
| | 2,6-dione |
| 459b | 3-(6-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2- |
| | (hydroxymethyl)piperazin-1-yl)methyl)piperidin-1-yl)-1-methyl-1H-indazol-3-yl)piperidine- |
| | 2,6-dione |
| 460 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-(hydroxymethyl)piperazin-1- |
| | yl]methyl]-1-piperidyl]-1-methyl-indazol-3-yl]piperidine-2,6-dione |
| 460a | 3-(7-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3- |
| | (hydroxymethyl)piperazin-1-yl)methyl)piperidin-1-yl)-1-methyl-1H-indazol-3-yl)piperidine- |
| | 2,6-dione |
| 460b | 3-(7-(4-(((R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3- |
| | (hydroxymethyl)piperazin-1-yl)methyl)piperidin-1-yl)-1-methyl-1H-indazol-3-yl)piperidine- |
| | 2,6-dione |
| 461 | 3-[7-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-fluoro-4-piperidyl]methyl]-4- |
| | piperidyl]-1-methyl-indazol-3-yl]piperidine-2,6-dione |
| 461a | 3-(7-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-4-fluoropiperidin-4- |
| | yl)methyl)piperidin-4-yl)-1-methyl-1H-indazol-3-yl)piperidine-2,6-dione |
| 462 | 3-[7-[4-[[4-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-2-pyridyl]piperazin-1-yl]methyl]-1- |
| | piperidyl]-1-methyl-indazol-3-yl]piperidine-2,6-dione |
| 462a | 3-(7-(4-((4-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyridin-2-yl)piperazin-1- |
| | yl)methyl)piperidin-1-yl)-1-methyl-1H-indazol-3-yl)piperidine-2,6-dione |
| 463 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-2-methyl-piperazin-1-yl]methyl]-1- |
| | piperidyl]-1-methyl-indazol-3-yl]piperidine-2,6-dione |
| 463a | 3-(6-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)-2-methylpiperazin-1- |
| | yl)methyl)piperidin-1-yl)-1-methyl-1H-indazol-3-yl)piperidine-2,6-dione |
| 464 | 3-[6-[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-6-methyl-2-azaspiro[3.5]nonan- |
| | 7-yl]amino]-1-methyl-indazol-3-yl]piperidine-2,6-dione |
| 464a | 3-(6-(((6S*,7S*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 464b | 3-(6-(((6R*,7S*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 464c | 3-(6-(((6S*,7R*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 464d | 3-(6-(((6R*,7R*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 465 | 3-[7-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-6-methyl-2-azaspiro[3.5]nonan- |
| | 7-yl]amino]-1-methyl-indazol-3-yl]piperidine-2,6-dione |
| 465a | 3-(7-(((6R*,7R*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 465b | 3-(7-(((6R*,7S*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 465c | 3-(7-(((6S*,7S*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 465d | 3-(7-(((6S*,7R*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)amino)-1-methyl-1H-indazol-3-yl)piperidine-2,6-dione |
| 466 | 3-[6-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-(hydroxymethyl)piperazin-1- |
| | yl]methyl]-1-piperidyl]-1-methyl-indazol-3-yl]piperidine-2,6-dione |
| 466a | 3-(6-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3- |
| | (hydroxymethyl)piperazin-1-yl)methyl)piperidin-1-yl)-1-methyl-1H-indazol-3-yl)piperidine- |
| | 2,6-dione |
| 466b | 3-(6-(4-(((R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3- |
| | (hydroxymethyl)piperazin-1-yl)methyl)piperidin-1-yl)-1-methyl-1H-indazol-3-yl)piperidine- |
| | 2,6-dione |
| 467 | 3-[6-[9-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3,9- |
| | diazaspiro[5.5]undecan-3-yl]-1-methyl-indazol-3-yl]piperidine-2,6-dione |
| 467a | 3-(6-(9-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3,9-diazaspiro[5.5]undecan-3-yl)-1-methyl-1H-indazol-3-yl)piperidine-2,6-dione |
| 468 | 3-[6-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,9-diazaspiro[5.5]undecan-9- |
| | yl]methyl]-1-methyl-indazol-3-yl]piperidine-2,6-dione |
| 468a | 3-(6-((9-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-3,9-diazaspiro[5.5]undecan-3- |
| | yl)methyl)-1-methyl-1H-indazol-3-yl)piperidine-2,6-dione |
| 469 | 3-[6-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,9-diazaspiro[5.5]undecan-9- |
| | yl]methyl]-1-piperidyl]-7-fluoro-1-methyl-indazol-3-yl]piperidine-2,6-dione |
| 469a | 3-(6-(4-((9-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3,9- |
| | diazaspiro[5.5]undecan-3-yl)methyl)piperidin-1-yl)-7-fluoro-1-methyl-1H-indazol-3- |
| | yl)piperidine-2,6-dione |
| 470 | 3-[6-[4-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]-1-methyl- |
| | ethyl]piperazin-1-yl]-1-methyl-indazol-3-yl]piperidine-2,6-dione |
| 470a | 3-(6-(4-(2-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4-yl)propan- |
| | 2-yl)piperazin-1-yl)-1-methyl-1H-indazol-3-yl)piperidine-2,6-dione |
| 471 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7- |
| | yl]amino]-7-fluoro-1-methyl-indazol-3-yl]piperidine-2,6-dione |
| 471a | 3-(6-((2-(5-chloro-4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)pyrimidin-2-yl)-2-azaspiro[3.5]nonan-7- |
| | yl)amino)-7-fluoro-1-methyl-1H-indazol-3-yl)piperidine-2,6-dione |
| 472 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-fluoro-4-piperidyl]methyl]-4- |
| | piperidyl]-1-methyl-indazol-3-yl]piperidine-2,6-dione |
| 472a | 3-(6-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-4-fluoropiperidin-4- |
| | yl)methyl)piperidin-4-yl)-1-methyl-1H-indazol-3-yl)piperidine-2,6-dione |
| 473 | 3-[6-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-azaspiro[5.5]undecan-9- |
| | yl]methyl]piperazin-1-yl]-1-methyl-indazol-3-yl]piperidine-2,6-dione |
| 473a | 3-(6-(4-((3-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3- |
| | azaspiro[5.5]undecan-9-yl)methyl)piperazin-1-yl)-1-methyl-1H-indazol-3-yl)piperidine-2,6- |
| | dione |
| 474 | 3-[6-[1-[[1-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-4-fluoro-4- |
| | piperidyl]methyl]-4-piperidyl]-1-methyl-indazol-3-yl]piperidine-2,6-dione |
| 474a | 3-(6-(1-((1-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)-4-fluoropiperidin-4- |
| | yl)methyl)piperidin-4-yl)-1-methyl-1H-indazol-3-yl)piperidine-2,6-dione |
| 475 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-2-methyl- |
| | piperazin-1-yl]-1-methyl-indazol-3-yl]piperidine-2,6-dione |
| 475a | 3-(7-((S)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-2-methylpiperazin-1-yl)-1-methyl-1H-indazol-3-yl)piperidine-2,6-dione |
| 475b | 3-(7-((R)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-2-methylpiperazin-1-yl)-1-methyl-1H-indazol-3-yl)piperidine-2,6-dione |
| 476 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-piperazin-1- |
| | yl]methyl]-1-piperidyl]-1-methyl-indazol-3-yl]piperidine-2,6-dione |
| 476a | 3-(7-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperazin-1- |
| | yl)methyl)piperidin-1-yl)-1-methyl-1H-indazol-3-yl)piperidine-2,6-dione |
| 476b | 3-(7-(4-(((R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperazin-1- |
| | yl)methyl)piperidin-1-yl)-1-methyl-1H-indazol-3-yl)piperidine-2,6-dione |
| 477 | 3-[6-[4-[[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-7-azaspiro[3.5]nonan-2- |
| | yl]methyl]piperazin-1-yl]-1-methyl-indazol-3-yl]piperidine-2,6-dione |
| 477a | 3-(6-(4-((7-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-7- |
| | azaspiro[3.5]nonan-2-yl)methyl)piperazin-1-yl)-1-methyl-1H-indazol-3-yl)piperidine-2,6- |
| | dione |
| 478 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]sulfonyl]piperazin-1- |
| | yl]-1-methyl-indazol-3-yl]piperidine-2,6-dione |
| 478a | 3-(7-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)sulfonyl)piperazin-1-yl)-1-methyl-1H-indazol-3-yl)piperidine-2,6-dione |
| 479 | 3-[7-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-4-piperidyl]methyl]-4-piperidyl]-1- |
| | methyl-indazol-3-yl]piperidine-2,6-dione |
| 479a | 3-(7-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperidin-4- |
| | yl)methyl)piperidin-4-yl)-1-methyl-1H-indazol-3-yl)piperidine-2,6-dione |
| 480 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,2-dimethyl-piperazin-1- |
| | yl]methyl]-1-piperidyl]-1-methyl-indazol-3-yl]piperidine-2,6-dione |
| 480a | 3-(7-(4-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,2- |
| | dimethylpiperazin-1-yl)methyl)piperidin-1-yl)-1-methyl-1H-indazol-3-yl)piperidine-2,6- |
| | dione |
| 481 | 3-[7-[4-[[5-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,5-diazabicyclo[2.2.1]heptan-2- |
| | yl]methyl]-1-piperidyl]-1-methyl-indazol-3-yl]piperidine-2,6-dione |
| 481a | 3-(7-(4-(((1R,4R)-5-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-2,5- |
| | diazabicyclo[2.2.1]heptan-2-yl)methyl)piperidin-1-yl)-1-methyl-1H-indazol-3-yl)piperidine- |
| | 2,6-dione |
| 481b | 3-(7-(4-(((1S,4S)-5-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,5- |
| | diazabicyclo[2.2.1]heptan-2-yl)methyl)piperidin-1-yl)-1-methyl-1H-indazol-3-yl)piperidine- |
| | 2,6-dione |
| 482 | 3-[7-[5-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-2,5- |
| | diazabicyclo[2.2.1]heptan-2-yl]-1-methyl-indazol-3-yl]piperidine-2,6-dione |
| 482a | 3-(7-((1R,4R)-5-((1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)piperidin- |
| | 4-yl)methyl)-2,5-diazabicyclo[2.2.1]heptan-2-yl)-1-methyl-1H-indazol-3-yl)piperidine-2,6- |
| | dione |
| 482b | 3-(7-((1S,4S)-5-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-2,5-diazabicyclo[2.2.1]heptan-2-yl)-1-methyl-1H-indazol-3-yl)piperidine-2,6- |
| | dione |
| 483 | 3-[7-[4-[[6-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,6-diazabicyclo[3.1.1]heptan-3- |
| | yl]methyl]-1-piperidyl]-1-methyl-indazol-3-yl]piperidine-2,6-dione |
| 483a | 3-(7-(4-((6-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3,6- |
| | diazabicyclo[3.1.1]heptan-3-yl)methyl)piperidin-1-yl)-1-methyl-1H-indazol-3-yl)piperidine- |
| | 2,6-dione |
| 484 | 3-[7-[4-[[4-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-3- |
| | (hydroxymethyl)piperazin-1-yl]methyl]-1-piperidyl]-1-methyl-indazol-3-yl]piperidine-2,6- |
| | dione |
| 484a | 3-(7-(4-(((R)-4-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)-3- |
| | (hydroxymethyl)piperazin-1-yl)methyl)piperidin-1-yl)-1-methyl-1H-indazol-3-yl)piperidine- |
| | 2,6-dione |
| 485 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-2-methyl-piperazin-1-yl]methyl]-1- |
| | piperidyl]-1-methyl-indazol-3-yl]piperidine-2,6-dione |
| 485a | 3-(7-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)-2-methylpiperazin-1- |
| | yl)methyl)piperidin-1-yl)-1-methyl-1H-indazol-3-yl)piperidine-2,6-dione |
| 486 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-4- |
| | piperidyl]methyl]piperazin-1-yl]-1-methyl-indazol-3-yl]piperidine-2,6-dione |
| 486a | 3-(6-(4-(((3S,4S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperazin-1-yl)-1-methyl-1H-indazol-3-yl)piperidine-2,6-dione |
| 486b | 3-(6-(4-(((3R,4S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperazin-1-yl)-1-methyl-1H-indazol-3-yl)piperidine-2,6-dione |
| 486c | 3-(6-(4-(((3S,4R)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperazin-1-yl)-1-methyl-1H-indazol-3-yl)piperidine-2,6-dione |
| 486d | 3-(6-(4-(((3R,4R)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | methylpiperidin-4-yl)methyl)piperazin-1-yl)-1-methyl-1H-indazol-3-yl)piperidine-2,6-dione |
| 487 | 3-[7-[4-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-4- |
| | piperidyl]methyl]piperazin-1-yl]-1-methyl-indazol-3-yl]piperidine-2,6-dione |
| 487a | 3-(7-(4-(((3S,4S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperazin-1-yl)-1-methyl-1H-indazol-3-yl)piperidine-2,6-dione |
| 487b | 3-(7-(4-(((3R,4R)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | methylpiperidin-4-yl)methyl)piperazin-1-yl)-1-methyl-1H-indazol-3-yl)piperidine-2,6-dione |
| 487c | 3-(7-(4-(((3S,4R)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperazin-1-yl)-1-methyl-1H-indazol-3-yl)piperidine-2,6-dione |
| 487d | 3-(7-(4-(((3R,4S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperazin-1-yl)-1-methyl-1H-indazol-3-yl)piperidine-2,6-dione |
| 488 | 3-[7-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,6-diazabicyclo[3.1.1]heptan-6- |
| | yl]methyl]-1-piperidyl]-1-methyl-indazol-3-yl]piperidine-2,6-dione |
| 488a | 3-(7-(4-((3-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3,6- |
| | diazabicyclo[3.1.1]heptan-6-yl)methyl)piperidin-1-yl)-1-methyl-1H-indazol-3-yl)piperidine- |
| | 2,6-dione |
| 489 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-hydroxy-4- |
| | piperidyl]methyl]piperazin-1-yl]-1-methyl-indazol-3-yl]piperidine-2,6-dione |
| 489a | 3-(7-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-4-hydroxypiperidin- |
| | 4-yl)methyl)piperazin-1-yl)-1-methyl-1H-indazol-3-yl)piperidine-2,6-dione |
| 490 | 3-[7-[4-[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-6,8-dihydro-5H- |
| | imidazo[1,5-a]pyrazin-1-yl]-1-piperidyl]-1-methyl-indazol-3-yl]piperidine-2,6-dione |
| 490a | 3-(7-(4-(7-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methyl-5,6,7,8- |
| | tetrahydroimidazo[1,5-a]pyrazin-1-yl)piperidin-1-yl)-1-methyl-1H-indazol-3-yl)piperidine- |
| | 2,6-dione |
| 491 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,5-dimethyl-piperazin-1- |
| | yl]methyl]-1-piperidyl]-1-methyl-indazol-3-yl]piperidine-2,6-dione |
| 491a | 3-(7-(4-(((2S,5R)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,5- |
| | dimethylpiperazin-1-yl)methyl)piperidin-1-yl)-1-methyl-1H-indazol-3-yl)piperidine-2,6- |
| | dione |
| 491b | 3-(7-(4-(((2R,5S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2,5- |
| | dimethylpiperazin-1-yl)methyl)piperidin-1-yl)-1-methyl-1H-indazol-3-yl)piperidine-2,6- |
| | dione |
| 492 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-6-methyl-2-azaspiro[3.5]nonan- |
| | 7-yl]oxy]-1-methyl-indazol-3-yl]piperidine-2,6-dione |
| 492a | 3-(6-(((6R*,7S*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)oxy)-1-methyl-1H-indazol-3-yl)piperidine-2,6-dione |
| 492b | 3-(6-(((6S*,7S*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)oxy)-1-methyl-1H-indazol-3-yl)piperidine-2,6-dione |
| 492c | 3-(6-(((6R*,7R*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)oxy)-1-methyl-1H-indazol-3-yl)piperidine-2,6-dione |
| 492d | 3-(6-(((6S*,7R*)-2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-6-methyl-2- |
| | azaspiro[3.5]nonan-7-yl)oxy)-1-methyl-1H-indazol-3-yl)piperidine-2,6-dione |
| 493 | 3-[7-[4-[[1-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-4-piperidyl]methyl]-3- |
| | methyl-piperazin-1-yl]-1-methyl-indazol-3-yl]piperidine-2,6-dione |
| 493a | 3-(7-((S)-4-((1-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)piperidin-4-yl)methyl)- |
| | 3-methylpiperazin-1-yl)-1-methyl-1H-indazol-3-yl)piperidine-2,6-dione |
| 493b | 3-(7-((R)-4-((1-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)piperidin-4-yl)methyl)- |
| | 3-methylpiperazin-1-yl)-1-methyl-1H-indazol-3-yl)piperidine-2,6-dione |
| 494 | 3-[6-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-3,9-diazaspiro[5.5]undecan-9- |
| | yl]methyl]-1-piperidyl]-1-methyl-indazol-3-yl]piperidine-2,6-dione |
| 494a | 3-(6-(4-((9-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)-3,9- |
| | diazaspiro[5.5]undecan-3-yl)methyl)piperidin-1-yl)-1-methyl-1H-indazol-3-yl)piperidine- |
| | 2,6-dione |
| 495 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-4-piperidyl]methyl]-4-piperidyl]-1- |
| | methyl-indazol-3-yl]piperidine-2,6-dione |
| 495a | 3-(6-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperidin-4- |
| | yl)methyl)piperidin-4-yl)-1-methyl-1H-indazol-3-yl)piperidine-2,6-dione |
| 496 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-4-piperidyl]methyl]-3-methyl- |
| | piperazin-1-yl]-1-methyl-indazol-3-yl]piperidine-2,6-dione |
| 496a | 3-[7-[(3R)-4-[[1-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]-2-pyridyl]-4-piperidyl]methyl]-3-methyl- |
| | piperazin-1-yl]-1-methyl-indazol-3-yl]piperidine-2,6-dione |
| 496b | 3-(7-((S)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)piperidin-4-yl)methyl)-3- |
| | methylpiperazin-1-yl)-1-methyl-1H-indazol-3-yl)piperidine-2,6-dione |
| 497 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-fluoro-4-piperidyl]methyl]-3- |
| | methyl-piperazin-1-yl]-1-methyl-indazol-3-yl]piperidine-2,6-dione |
| 497a | 3-[7-[(3S)-4-[[1-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]-4-fluoro-4- |
| | piperidyl]methyl]-3-methyl-piperazin-1-yl]-1-methyl-indazol-3-yl]piperidine-2,6-dione |
| 498 | 3-[7-[4-[[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4,7-diazaspiro[2.5]octan-4- |
| | yl]methyl]-1-piperidyl]-1-methyl-indazol-3-yl]piperidine-2,6-dione |
| 498a | 3-[7-[4-[[7-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4,7-diazaspiro[2.5]octan-4- |
| | yl]methyl]-1-piperidyl]-1-methyl-indazol-3-yl]piperidine-2,6-dione |
| 499 | 3-[7-[4-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]ethyl]piperazin-1- |
| | yl]-1-methyl-indazol-3-yl]piperidine-2,6-dione |
| 499a | 3-(7-(4-((R*)-1-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)ethyl)piperazin-1-yl)-1-methyl-1H-indazol-3-yl)piperidine-2,6-dione |
| 499b | 3-(7-(4-((S*)-1-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)ethyl)piperazin-1-yl)-1-methyl-1H-indazol-3-yl)piperidine-2,6-dione |
| 500 | 3-[7-[4-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]-1-hydroxy-ethyl]-1- |
| | piperidyl]-1-methyl-indazol-3-yl]piperidine-2,6-dione |
| 500a | 3-[7-[4-[(1S*)-1-[1-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-4-piperidyl]-1- |
| | hydroxy-ethyl]-1-piperidyl]-1-methyl-indazol-3-yl]piperidine-2,6-dione |
| 500b | 3-(7-(4-((R*)-1-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4-yl)-1- |
| | hydroxyethyl)piperidin-1-yl)-1-methyl-1H-indazol-3-yl)piperidine-2,6-dione |
| 501 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7- |
| | yl]amino]-5-fluoro-1-methyl-indazol-3-yl]piperidine-2,6-dione |
| 501a | 3-[6-[[2-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2-azaspiro[3.5]nonan-7- |
| | yl]amino]-5-fluoro-1-methyl-indazol-3-yl]piperidine-2,6-dione |
| 502 | 3-[7-[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7- |
| | yl]amino]-4-fluoro-1-methyl-indazol-3-yl]piperidine-2,6-dione |
| 502a | 3-[7-[[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2-azaspiro[3.5]nonan-7- |
| | yl]amino]-4-fluoro-1-methyl-indazol-3-yl]piperidine-2,6-dione |
| 503 | 3-[6-[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2,7-diazaspiro[3.5]nonane-7- |
| | carbonyl]-1-methyl-indazol-3-yl]piperidine-2,6-dione |
| 503a | 3-[6-[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2,7-diazaspiro[3.5]nonane-7- |
| | carbonyl]-1-methyl-indazol-3-yl]piperidine-2,6-dione |
| 504 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-hydroxy-4-piperidyl]methyl]- |
| | 4-piperidyl]-1-methyl-indazol-3-yl]piperidine-2,6-dione |
| 504a | 3-[6-[1-[[1-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-hydroxy-4-piperidyl]methyl]- |
| | 4-piperidyl]-1-methyl-indazol-3-yl]piperidine-2,6-dione |
| 505 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3,3- |
| | dimethyl-piperazin-1-yl]-1-methyl-indazol-3-yl]piperidine-2,6-dione |
| 505a | 3-(6-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3,3-dimethylpiperazin-1-yl)-1-methyl-1H-indazol-3-yl)piperidine-2,6-dione |
| 506 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3,5- |
| | dimethyl-piperazin-1-yl]-1-methyl-indazol-3-yl]piperidine-2,6-dione |
| 506a | 3-(7-((3R,5S)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3,5-dimethylpiperazin-1-yl)-1-methyl-1H-indazol-3-yl)piperidine-2,6-dione |
| 506b | 3-(7-((3S,5S)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3,5-dimethylpiperazin-1-yl)-1-methyl-1H-indazol-3-yl)piperidine-2,6-dione |
| 507 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-4-piperidyl]- |
| | 7-fluoro-1-methyl-indazol-3-yl]piperidine-2,6-dione |
| 507a | 3-(6-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperidin-4-yl)-7-fluoro-1-methyl-1H-indazol-3-yl)piperidine-2,6-dione |
| 508 | 3-[6-[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-azaspiro[5.5]undecan-9- |
| | yl]amino]-1-methyl-indazol-3-yl]piperidine-2,6-dione |
| 508a | 3-[6-[[3-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-3-azaspiro[5.5]undecan-9- |
| | yl]amino]-1-methyl-indazol-3-yl]piperidine-2,6-dione |
| 509 | 3-[7-[6-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3,6- |
| | diazabicyclo[3.1.1]heptan-3-yl]-1-methyl-indazol-3-yl]piperidine-2,6-dione |
| 509a | 3-[7-[6-[[1-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-piperidyl]methyl]-3,6- |
| | diazabicyclo[3.1.1]heptan-3-yl]-1-methyl-indazol-3-yl]piperidine-2,6-dione |
| 510 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3,3- |
| | dimethyl-piperazin-1-yl]-1-methyl-indazol-3-yl]piperidine-2,6-dione |
| 510a | 3-(7-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3,3-dimethylpiperazin-1-yl)-1-methyl-1H-indazol-3-yl)piperidine-2,6-dione |
| 511 | 3-[6-[4-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]-1-hydroxy-ethyl]-1- |
| | piperidyl]-1-methyl-indazol-3-yl]piperidine-2,6-dione |
| 511a | 3-[6-[4-[(1S*)-1-[1-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-4-piperidyl]-1- |
| | hydroxy-ethyl]-1-piperidyl]-1-methyl-indazol-3-yl]piperidine-2,6-dione |
| 511b | 3-[6-[4-[(1R*)-1-[1-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-4-piperidyl]-1- |
| | hydroxy-ethyl]-1-piperidyl]-1-methyl-indazol-3-yl]piperidine-2,6-dione |
| 512 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-(1-hydroxy-1-methyl- |
| | ethyl)piperazin-1-yl]methyl]-1-piperidyl]-1-methyl-indazol-3-yl]piperidine-2,6-dione |
| 512a | 3-[7-[4-[(2S)-4-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]-2-(1-hydroxy-1-methyl- |
| | ethyl)piperazin-1-yl]methyl]-1-piperidyl]-1-methyl-indazol-3-yl]piperidine-2,6-dione |
| 512b | 3-[7-[4-[[(2R)-4-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]-2-(1-hydroxy-1-methyl- |
| | ethyl)piperazin-1-yl]methyl]-1-piperidyl]-1-methyl-indazol-3-yl]piperidine-2,6-dione |
| 513 | 3-[7-[4-[[1-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-2-pyridyl]-4-piperidyl]methyl]-3- |
| | methyl-piperazin-1-yl]-1-methyl-indazol-3-yl]piperidine-2,6-dione |
| 513a | 3-[7-[(3S)-4-[[1-[4-[[(2S)-2-cyclopropyl-3,3-difluoro-7-methyl-6-oxo-2,4-dihydro-1H- |
| | [1,4]oxazepino[2,3-c]quinolin-10-yl]amino]-5-fluoro-2-pyridyl]-4-piperidyl]methyl]-3- |
| | methyl-piperazin-1-yl]-1-methyl-indazol-3-yl]piperidine-2,6-dione |
| 514 | 3-[7-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-hydroxy-4-piperidyl]methyl]- |
| | 4-piperidyl]-1-methyl-indazol-3-yl]piperidine-2,6-dione |
| 514a | 3-[7-[1-[[1-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-hydroxy-4-piperidyl]methyl]- |
| | 4-piperidyl]-1-methyl-indazol-3-yl]piperidine-2,6-dione |
| 516 | 3-[6-[4-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7- |
| | yl]methyl]piperazin-1-yl]-1-methyl-indazol-3-yl]piperidine-2,6-dione |
| 516a | 3-(6-(4-((2-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-2- |
| | azaspiro[3.5]nonan-7-yl)methyl)piperazin-1-yl)-1-methyl-1H-indazol-3-yl)piperidine-2,6- |
| | dione |
| 518 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.3]heptan-6- |
| | yl]amino]-1-methyl-indazol-3-yl]piperidine-2,6-dione |
| 518a | 3-[6-[[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2-azaspiro[3.3]heptan-6- |
| | yl]amino]-1-methyl-indazol-3-yl]piperidine-2,6-dione |
| 519 | 3-[6-[7-[[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]amino]-2-azaspiro[3.5]nonan-2- |
| | yl]-1-methyl-indazol-3-yl]piperidine-2,6-dione |
| 519a | 3-[6-[7-[[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]amino]-2-azaspiro[3.5]nonan-2- |
| | yl]-1-methyl-indazol-3-yl]piperidine-2,6-dione |
| 520 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.3]heptan-6-yl]- |
| | methyl-amino]-1-methyl-indazol-3-yl]piperidine-2,6-dione |
| 520a | 3-[6-[[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2-azaspiro[3.3]heptan-6-yl]- |
| | methyl-amino]-1-methyl-indazol-3-yl]piperidine-2,6-dione |
| 521 | 3-[6-[4-[[4-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-2-pyridyl]piperazin-1-yl]methyl]-1- |
| | piperidyl]-1-methyl-indazol-3-yl]piperidine-2,6-dione |
| 521a | 3-[6-[4-[4-[4-[[(2S)-2-cyclopropyl-3,3-difluoro-7-methyl-6-oxo-2,4-dihydro-1H- |
| | [1,4]oxazepino[2,3-c]quinolin-10-yl]amino]-5-fluoro-2-pyridyl]piperazin-1-yl]methyl]-1- |
| | piperidyl]-1-methyl-indazol-3-yl]piperidine-2,6-dione |
| 522 | 3-[6-[1-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]ethyl]-4-piperidyl]- |
| | 1-methyl-indazol-3-yl]piperidine-2,6-dione |
| 522a | 3-(6-(1-((S*)-1-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)ethyl)piperidin-4-yl)-1-methyl-1H-indazol-3-yl)piperidine-2,6-dione |
| 522b | 3-(6-(1-((R*)-1-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)ethyl)piperidin-4-yl)-1-methyl-1H-indazol-3-yl)piperidine-2,6-dione |
| 523 | 3-[6-[4-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]ethyl]piperazin-1- |
| | yl]-1-methyl-indazol-3-yl]piperidine-2,6-dione |
| 523a | 3-(6-(4-((R*)-1-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)ethyl)piperazin-1-yl)-1-methyl-1H-indazol-3-yl)piperidine-2,6-dione |
| 523b | 3-(6-(4-((S*)-1-(1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)ethyl)piperazin-1-yl)-1-methyl-1H-indazol-3-yl)piperidine-2,6-dione |
| 524 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.3]heptan-6-yl]oxy]- |
| | 1-methyl-indazol-3-yl]piperidine-2,6-dione |
| 524a | 3-[6-[[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2-azaspiro[3.3]heptan-6-yl]oxy]- |
| | 1-methyl-indazol-3-yl]piperidine-2,6-dione |
| 525 | 3-[6-[1-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-4-piperidyl]methyl]-4- |
| | piperidyl]-1-methyl-indazol-3-yl]piperidine-2,6-dione |
| 525a | 3-(6-(1-(((3S,4S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperidin-4-yl)-1-methyl-1H-indazol-3-yl)piperidine-2,6-dione |
| 525b | 3-(6-(1-(((3R,4R)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | methylpiperidin-4-yl)methyl)piperidin-4-yl)-1-methyl-1H-indazol-3-yl)piperidine-2,6-dione |
| 525c | 3-(6-(1-(((3R,4S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperidin-4-yl)-1-methyl-1H-indazol-3-yl)piperidine-2,6-dione |
| 525d | 3-(6-(1-(((3S,4R)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperidin-4-yl)-1-methyl-1H-indazol-3-yl)piperidine-2,6-dione |
| 526 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-4-hydroxy- |
| | 4-piperidyl]-1-methyl-indazol-3-yl]piperidine-2,6-dione |
| 526a | 3-(6-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-4-hydroxypiperidin-4-yl)-1-methyl-1H-indazol-3-yl)piperidine-2,6-dione |
| 527 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]pyrrolidin-3-yl]methyl]piperazin- |
| | 1-yl]-1-methyl-indazol-3-yl]piperidine-2,6-dione |
| 527a | 3-[6-[4-[[(3S)-1-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]pyrrolidin-3- |
| | yl]methyl]piperazin-1-yl]-1-methyl-indazol-3-yl]piperidine-2,6-dione |
| 527b | 3-[6-[4-[[(3R)-1-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]pyrrolidin-3- |
| | yl]methyl]piperazin-1-yl]-1-methyl-indazol-3-yl]piperidine-2,6-dione |
| 528 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]pyrrolidin-3-yl]methyl]-3-methyl- |
| | piperazin-1-yl]-1-methyl-indazol-3-yl]piperidine-2,6-dione |
| 528a | 3-(6-((S)-4-(((S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)pyrrolidin-3- |
| | yl)methyl)-3-methylpiperazin-1-yl)-1-methyl-1H-indazol-3-yl)piperidine-2,6-dione |
| 529 | 3-[6-[3-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-methyl-piperazin-1- |
| | yl]methyl]pyrrolidin-1-yl]-1-methyl-indazol-3-yl]piperidine-2,6-dione |
| 529a | 3-[6-[(3R)-3-[[(2S)-4-[5-chloro-4-[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-2-methyl-piperazin- |
| | 1-yl]methyl]pyrrolidin-1-yl]-1-methyl-indazol-3-yl]piperidine-2,6-dione |
| 529b | 3-[6-[(3S)-3-[(2S)-4-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-2-methyl-piperazin- |
| | 1-yl]methyl]pyrrolidin-1-yl]-1-methyl-indazol-3-yl]piperidine-2,6-dione |
| 529c | 3-[6-[(3R)-3-[(2R)-4-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-2-methyl-piperazin- |
| | 1-yl]methyl]pyrrolidin-1-yl]-1-methyl-indazol-3-yl]piperidine-2,6-dione |
| 530 | 3-[7-[3-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3,6- |
| | diazabicyclo[3.1.1]heptan-6-yl]-1-methyl-indazol-3-yl]piperidine-2,6-dione |
| 530a | 3-[7-[3-[1-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-piperidyl]methyl]-3,6- |
| | diazabicyclo[3.1.1]heptan-6-yl]-1-methyl-indazol-3-yl]piperidine-2,6-dione |
| 531 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-4-piperidyl]- |
| | 5-fluoro-1-methyl-indazol-3-yl]piperidine-2,6-dione |
| 531a | 3-(6-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperidin-4-yl)-5-fluoro-1-methyl-1H-indazol-3-yl)piperidine-2,6-dione |
| 532 | 3-[6-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazine-1-carbonyl]piperazin- |
| | 1-yl]-1-methyl-indazol-3-yl]piperidine-2,6-dione |
| 532a | 3-[6-[4-[4-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]piperazine-1-carbonyl]piperazin- |
| | 1-yl]-1-methyl-indazol-3-yl]piperidine-2,6-dione |
| 533 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-4-fluoro-4- |
| | piperidyl]-1-methyl-indazol-3-yl]piperidine-2,6-dione |
| 533a | 3-(6-(1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-4-fluoropiperidin-4-yl)-1-methyl-1H-indazol-3-yl)piperidine-2,6-dione |
| 534 | 3-[6-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-6-(trifluoromethyl)-2- |
| | azaspiro[3.5]nonan-7-yl]amino]-1-methyl-indazol-3-yl]piperidine-2,6-dione |
| 534a | 3-[6-[[(6S*,7R*)-2-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-6-(trifluoromethyl)- |
| | 2-azaspiro[3.5]nonan-7-yl]amino]-1-methyl-indazol-3-yl]piperidine-2,6-dione |
| 534b | 3-[6-[[(6S*,7S*)-2-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-6-(trifluoromethyl)- |
| | 2-azaspiro[3.5]nonan-7-yl]amino]-1-methyl-indazol-3-yl]piperidine-2,6-dione |
| 534c | 3-[6-[[(6R*,7S*)-2-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-6-(trifluoromethyl)- |
| | 2-azaspiro[3.5]nonan-7-yl]amino]-1-methyl-indazol-3-yl]piperidine-2,6-dione |
| 534d | 3-[6-[[(6R*,7R*)-2-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-6-(trifluoromethyl)- |
| | 2-azaspiro[3.5]nonan-7-yl]amino]-1-methyl-indazol-3-yl]piperidine-2,6-dione |
| 535 | 3-[7-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7- |
| | yl]amino]-6-fluoro-1-methyl-indazol-3-yl]piperidine-2,6-dione |
| 535a | 3-[7-[[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2-azaspiro[3.5]nonan-7- |
| | yl]amino]-6-fluoro-1-methyl-indazol-3-yl]piperidine-2,6-dione |
| 536 | 3-[6-[4-[[1-[5-chloro-4-[(2-cyclopropyl-3,3,11-trifluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazin-1- |
| | yl]-1-methyl-indazol-3-yl]piperidine-2,6-dione |
| 536a | 3-[6-[4-[[1-[5-chloro-4-[[(2S)-2-cyclopropyl-3,3,11-trifluoro-7-methyl-6-oxo-2,4-dihydro- |
| | 1H-[1,4]oxazepino[2,3-c]quinolin-10-yl]amino]pyrimidin-2-yl]-4- |
| | piperidyl]methyl]piperazin-1-yl]-1-methyl-indazol-3-yl]piperidine-2,6-dione |
| 537 | 3-[6-[1-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazine-1-carbonyl]-4- |
| | piperidyl]-1-methyl-indazol-3-yl]piperidine-2,6-dione |
| 537a | 3-[6-[1-[4-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]piperazine-1-carbonyl]-4- |
| | piperidyl]-1-methyl-indazol-3-yl]piperidine-2,6-dione |
| 538 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-2-methyl-4- |
| | piperidyl]-1-methyl-indazol-3-yl]piperidine-2,6-dione |
| 538a | 3-[6-[(2S,4R)-1-[1-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-4-piperidyl]methyl]- |
| | 2-methyl-4-piperidyl]-1-methyl-indazol-3-yl]piperidine-2,6-dione |
| 538b | 3-[6-[(2R,4S)-1-[[1-[5-chloro-4-[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-4-piperidyl]methyl]- |
| | 2-methyl-4-piperidyl]-1-methyl-indazol-3-yl]piperidine-2,6-dione |
| 538c | 3-[6-[(2S,4S)-1-[[1-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-4-piperidyl]methyl]- |
| | 2-methyl-4-piperidyl]-1-methyl-indazol-3-yl]piperidine-2,6-dione |
| 539 | 3-[6-[1-[1-[5-chloro-4-[(2-cyclopropyl-3,3,11-trifluoro-7-methyl-6-oxo-2,4-dihydro-1H- |
| | [1,4]oxazepino[2,3-c]quinolin-10-yl)amino]pyrimidin-2-yl]-4-piperidyl]methyl]-4-piperidyl]- |
| | 1-methyl-indazol-3-yl]piperidine-2,6-dione |
| 539a | 3-[6-[1-[[1-[5-chloro-4-[[(2S)-2-cyclopropyl-3,3,11-trifluoro-7-methyl-6-oxo-2,4-dihydro- |
| | 1H-[1,4]oxazepino[2,3-c]quinolin-10-yl]amino]pyrimidin-2-yl]-4-piperidyl]methyl]-4- |
| | piperidyl]-1-methyl-indazol-3-yl]piperidine-2,6-dione |
| 540 | 3-[7-[4-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazine-1-carbonyl]piperazin- |
| | 1-yl]-1-methyl-indazol-3-yl]piperidine-2,6-dione |
| 540a | 3-[7-[4-[4-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]piperazine-1-carbonyl]piperazin- |
| | 1-yl]-1-methyl-indazol-3-yl]piperidine-2,6-dione |
| 541 | 3-[7-[1-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazine-1-carbonyl]-4- |
| | piperidyl]-1-methyl-indazol-3-yl]piperidine-2,6-dione |
| 541a | 3-[7-[1-[4-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]piperazine-1-carbonyl]-4- |
| | piperidyl]-1-methyl-indazol-3-yl]piperidine-2,6-dione |
| 542 | 3-[6-[4-[3-[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]cyclobutoxy]-1- |
| | piperidyl]-1-methyl-indazol-3-yl]piperidine-2,6-dione |
| 542a | 3-(6-(4-((1r,3r)-3-(4-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)piperazin- |
| | 1-yl)cyclobutoxy)piperidin-1-yl)-1-methyl-1H-indazol-3-yl)piperidine-2,6-dione |
| 543 | 3-[6-[4-[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-6,8-dihydro-5H- |
| | imidazo[1,5-a]pyrazin-1-yl]-1-piperidyl]-1-methyl-indazol-3-yl]piperidine-2,6-dione |
| 543a | 3-[6-[4-[7-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-3-methyl-6,8-dihydro-5H- |
| | imidazo[1,5-a]pyrazin-1-yl]-1-piperidyl]-1-methyl-indazol-3-yl]piperidine-2,6-dione |
| 544 | 3-[6-[4-[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-6,8-dihydro-5H- |
| | [1,2,4]triazolo[4,3-a]pyrazin-3-yl]-1-piperidyl]-1-methyl-indazol-3-yl]piperidine-2,6-dione |
| 544a | 3-[6-[4-[7-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-6,8-dihydro-5H- |
| | [1,2,4]triazolo[4,3-a]pyrazin-3-yl]-1-piperidyl]-1-methyl-indazol-3-yl]piperidine-2,6-dione |
| 545 | 3-[6-[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,4,6,7,9,9a-hexahydro-1H- |
| | pyrazino[1,2-a]pyrazin-8-yl]-1-methyl-indazol-3-yl]piperidine-2,6-dione |
| 545a | 3-[6-[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-3,4,6,7,9,9a-hexahydro-1H- |
| | pyrazino[1,2-a]pyrazin-8-yl]-1-methyl-indazol-3-yl]piperidine-2,6-dione |
| 546 | 3-[6-[3-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]-6,8-dihydro-5H- |
| | [1,2,4]triazolo[4,3-a]pyrazin-7-yl]-1-methyl-indazol-3-yl]piperidine-2,6-dione |
| 546a | 3-[6-[3-[1-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-piperidyl]-6,8-dihydro-5H- |
| | [1,2,4]triazolo[4,3-a]pyrazin-7-yl]-1-methyl-indazol-3-yl]piperidine-2,6-dione |
| 547 | 3-[6-[4-[3-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4- |
| | piperidyl]oxy]cyclobutyl]piperazin-1-yl]-1-methyl-indazol-3-yl]piperidine-2,6-dione |
| 547a | 3-(6-(4-((1r,3r)-3-((1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)piperidin- |
| | 4-yl)oxy)cyclobutyl)piperazin-1-yl)-1-methyl-1H-indazol-3-yl)piperidine-2,6-dione |
| 548 | 3-[6-[3-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]piperazin-1-yl]methyl]pyrrolidin- |
| | 1-yl]-1-methyl-indazol-3-yl]piperidine-2,6-dione |
| 548a | 3-(6-((S)-3-((4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperazin-1- |
| | yl)methyl)pyrrolidin-1-yl)-1-methyl-1H-indazol-3-yl)piperidine-2,6-dione |
| 548b | 3-[6-[(3R)-3-[[4-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]piperazin-1- |
| | yl]methyl]pyrrolidin-1-yl]-1-methyl-indazol-3-yl]piperidine-2,6-dione |
| 549 | 3-[7-[4-[[4-[5-chloro-4-[(2-cyclopropyl-3,3-difluoro-7-methyl-6-oxo-2,4-dihydro-1H- |
| | [1,4]oxazepino[2,3-c]quinolin-10-yl)amino]-2-pyridyl]-2-(hydroxymethyl)piperazin-1- |
| | yl]methyl]-1-piperidyl]-1-methyl-indazol-3-yl]piperidine-2,6-dione |
| 549a | 3-(7-(4-(((S)-4-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyridin-2-yl)-2- |
| | (hydroxymethyl)piperazin-1-yl)methyl)piperidin-1-yl)-1-methyl-1H-indazol-3-yl)piperidine- |
| | 2,6-dione |
| 550 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazin-1- |
| | yl]-5-fluoro-1-methyl-indazol-3-yl]piperidine-2,6-dione |
| 550a | 3-(6-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperazin-1-yl)-5-fluoro-1-methyl-1H-indazol-3-yl)piperidine-2,6-dione |
| 551 | 3-[6-[4-[3-[5-chloro-4-[(2-cyclopropyl-3,3,11-trifluoro-7-methyl-6-oxo-2,4-dihydro-1H- |
| | [1,4]oxazepino[2,3-c]quinolin-10-yl)amino]pyrimidin-2-yl]-3,9-diazaspiro[5.5]undecan-9- |
| | yl]methyl]-1-piperidyl]-1-methyl-indazol-3-yl]piperidine-2,6-dione |
| 551a | 3-[6-[4-[[3-[5-chloro-4-[[(2S)-2-cyclopropyl-3,3,11-trifluoro-7-methyl-6-oxo-2,4-dihydro- |
| | 1H-[1,4]oxazepino[2,3-c]quinolin-10-yl]amino]pyrimidin-2-yl]-3,9-diazaspiro[5.5]undecan- |
| | 9-yl]methyl]-1-piperidyl]-1-methyl-indazol-3-yl]piperidine-2,6-dione |
| 552 | 3-[6-[4-[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-7-azaspiro[3.5]nonan-2- |
| | yl]piperazin-1-yl]-1-methyl-indazol-3-yl]piperidine-2,6-dione |
| 552a | 3-[6-[4-[7-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-7-azaspiro[3.5]nonan-2- |
| | yl]piperazin-1-yl]-1-methyl-indazol-3-yl]piperidine-2,6-dione |
| 553 | 3-[7-[4-[7-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-7-azaspiro[3.5]nonan-2- |
| | yl]piperazin-1-yl]-1-methyl-indazol-3-yl]piperidine-2,6-dione |
| 553a | 3-[7-[4-[7-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-7-azaspiro[3.5]nonan-2- |
| | yl]piperazin-1-yl]-1-methyl-indazol-3-yl]piperidine-2,6-dione |
| 554 | 3-[6-[4-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-4- |
| | piperidyl]methyl]piperazin-1-yl]-7-fluoro-1-methyl-indazol-3-yl]piperidine-2,6-dione |
| 554a | 3-[6-[4-[[(3S,4S)-1-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-3-methyl-4- |
| | piperidyl]methyl]piperazin-1-yl]-7-fluoro-1-methyl-indazol-3-yl]piperidine-2,6-dione |
| 555 | 3-[6-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-azabicyclo[3.2.1]octan-6- |
| | yl]methyl]piperazin-1-yl]-1-methyl-indazol-3-yl]piperidine-2,6-dione |
| 555a | 3-[6-[4-[3-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-3-azabicyclo[3.2.1]octan-6- |
| | yl]methyl]piperazin-1-yl]-1-methyl-indazol-3-yl]piperidine-2,6-dione |
| 556 | 3-[6-[4-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-8-oxa-3-azabicyclo[3.2.1]octan- |
| | 6-yl]methyl]piperazin-1-yl]-1-methyl-indazol-3-yl]piperidine-2,6-dione |
| 556a | 3-[6-[4-[[3-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-8-oxa-3-azabicyclo[3.2.1]octan- |
| | 6-yl]methyl]piperazin-1-yl]-1-methyl-indazol-3-yl]piperidine-2,6-dione |
| 557 | 3-[6-[4-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azabicyclo[4.1.0]heptan-5- |
| | yl]methyl]piperazin-1-yl]-1-methyl-indazol-3-yl]piperidine-2,6-dione |
| 557a | 3-[6-[4-[[(1SR,5SR,6SR)-2-[5-chloro-4-[[(2S)-2-cyclopropyl-3,3-difluoro-7-methyl-6-oxo- |
| | 2,4-dihydro-1H-[1,4]oxazepino[2,3-c]quinolin-10-yl]amino]pyrimidin-2-yl]-2- |
| | azabicyclo[4.1.0]heptan-5-yl]methyl]piperazin-1-yl]-1-methyl-indazol-3-yl]piperidine-2,6- |
| | dione |
| 558 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-methyl-4-piperidyl]methyl]-4- |
| | piperidyl]-1-methyl-indazol-3-yl]piperidine-2,6-dione |
| 558a | 3-[6-[1-[1-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-methyl-4-piperidyl]methyl]-4- |
| | piperidyl]-1-methyl-indazol-3-yl]piperidine-2,6-dione |
| 559 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3- |
| | (hydroxymethyl)piperazin-1-yl]-1-methyl-indazol-3-yl]piperidine-2,6-dione |
| 559a | 3-[7-[(3S)-4-[[1-[5-chloro-4-[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]-4-piperidyl]methyl]-3- |
| | (hydroxymethyl)piperazin-1-yl]-1-methyl-indazol-3-yl]piperidine-2,6-dione |
| 559b | 3-[7-[(3R)-4-[[1-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]-4-piperidyl]methyl]-3- |
| | (hydroxymethyl)piperazin-1-yl]-1-methyl-indazol-3-yl]piperidine-2,6-dione |
| 560 | 3-[6-[4-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3- |
| | (hydroxymethyl)piperazin-1-yl]-1-methyl-indazol-3-yl]piperidine-2,6-dione |
| 560a | 3-[6-[(3R)-4-[1-[5-chloro-4-[[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]-4-piperidyl]methyl]-3- |
| | (hydroxymethyl)piperazin-1-yl]-1-methyl-indazol-3-yl]piperidine-2,6-dione |
| 560b | 3-(6-((S)-4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)-3-(hydroxymethyl)piperazin-1-yl)-1-methyl-1H-indazol-3-yl)piperidine-2,6-dione |
| 561 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]piperazin-1- |
| | yl]-7-fluoro-1-methyl-indazol-3-yl]piperidine-2,6-dione |
| 561a | 3-(6-(4-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)piperazin-1-yl)-7-fluoro-1-methyl-1H-indazol-3-yl)piperidine-2,6-dione |
| 562 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-3-oxo- |
| | piperazin-1-yl]-1-methyl-indazol-3-yl]piperidine-2,6-dione |
| 562a | 3-[6-[4-[[1-[5-chloro-4-[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-piperidyl]methyl]-3-oxo- |
| | piperazin-1-yl]-1-methyl-indazol-3-yl]piperidine-2,6-dione |
| 563 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methyl-4- |
| | piperidyl]methyl]piperazin-1-yl]-5-fluoro-1-methyl-indazol-3-yl]piperidine-2,6-dione |
| 563a | 3-(6-(4-(((3S,4S)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3-methylpiperidin-4- |
| | yl)methyl)piperazin-1-yl)-5-fluoro-1-methyl-1H-indazol-3-yl)piperidine-2,6-dione |
| 563b | 3-(6-(4-(((3R,4R)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | methylpiperidin-4-yl)methyl)piperazin-1-yl)-5-fluoro-1-methyl-1H-indazol-3-yl)piperidine- |
| | 2,6-dione |
| 564 | 3-[7-[4-[1-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-3-methyl-4- |
| | piperidyl]methyl]piperazin-1-yl]-1-methyl-indazol-3-yl]piperidine-2,6-dione |
| 564a | 3-(7-(4-(((3S,4S)-1-(4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)-5-fluoropyrimidin-2-yl)-3- |
| | methylpiperidin-4-yl)methyl)piperazin-1-yl)-1-methyl-1H-indazol-3-yl)piperidine-2,6-dione |
| 565 | 3-[6-[4-[[3-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-3,9- |
| | diazaspiro[5.5]undecan-9-yl]methyl]-1-piperidyl]-7-fluoro-1-methyl-indazol-3-yl]piperidine- |
| | 2,6-dione |
| 565a | 3-(6-(4-((9-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)-3,9- |
| | diazaspiro[5.5]undecan-3-yl)methyl)piperidin-1-yl)-7-fluoro-1-methyl-1H-indazol-3- |
| | yl)piperidine-2,6-dione |
| 566 | 3-[6-[4-[[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azaspiro[3.5]nonan-7-yl]oxy]- |
| | 1-piperidyl]-1-methyl-indazol-3-yl]piperidine-2,6-dione |
| 566a | 3-[6-[4-[[2-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-2-azaspiro[3.5]nonan-7-yl]oxy]- |
| | 1-piperidyl]-1-methyl-indazol-3-yl]piperidine-2,6-dione |
| 568 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]azepan-4-yl]methyl]piperazin-1- |
| | yl]-1-methyl-indazol-3-yl]piperidine-2,6-dione |
| 568a | 3-[7-[4-[[(4R*)-1-[5-chloro-4-[[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]azepan-4- |
| | yl]methyl]piperazin-1-yl]-1-methyl-indazol-3-yl]piperidine-2,6-dione |
| 568b | 3-[7-[4-[[(4S*)-1-[5-chloro-4-[(2S)-2-cyclopropyl-3,3-difluoro-7-methyl-6-oxo-2,4-dihydro- |
| | 1H-[1,4]oxazepino[2,3-c]quinolin-10-yl]amino]pyrimidin-2-yl]azepan-4- |
| | yl]methyl]piperazin-1-yl]-1-methyl-indazol-3-yl]piperidine-2,6-dione |
| 569 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-1,4- |
| | diazepan-1-yl]-1-methyl-indazol-3-yl]piperidine-2,6-dione |
| 569a | 3-[6-[4-[1-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-piperidyl]methyl]-1,4- |
| | diazepan-1-yl]-1-methyl-indazol-3-yl]piperidine-2,6-dione |
| 570 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methylJazetidin-3- |
| | yl]oxy-1-methyl-indazol-3-yl]piperidine-2,6-dione |
| 570a | 3-[6-[1-[[1-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-piperidyl]methyl]azetidin-3- |
| | ylJoxy-1-methyl-indazol-3-yl]piperidine-2,6-dione |
| 572 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-methoxy-4- |
| | piperidyl]methyl]piperazin-1-yl]-1-methyl-indazol-3-yl]piperidine-2,6-dione |
| 572a | 3-[6-[4-[[1-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-3-methoxy-4- |
| | piperidyl]methyl]piperazin-1-yl]-1-methyl-indazol-3-yl]piperidine-2,6-dione |
| 573 | 3-[6-[4-[2-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-2-azabicyclo[3.1.0]hexan-5- |
| | yl]methyl]piperazin-1-yl]-1-methyl-indazol-3-yl]piperidine-2,6-dione |
| 573a | 3-[6-[4-[(1RS,5SR)-2-[5-chloro-4-[(2S)-2-cyclopropyl-3,3-difluoro-7-methyl-6-oxo-2,4- |
| | dihydro-1H-[1,4]oxazepino[2,3-c]quinolin-10-yl]amino]pyrimidin-2-yl]-2- |
| | azabicyclo[3.1.0]hexan-5-yl]methyl]piperazin-1-yl]-1-methyl-indazol-3-yl]piperidine-2,6- |
| | dione |
| 574 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]-1,4- |
| | diazepan-1-yl]-1-methyl-indazol-3-yl]piperidine-2,6-dione |
| 574a | 3-[7-[4-[[1-[5-chloro-4-[[(2S)-2-cyclopropyl-3,3-difluoro-7-methyl-6-oxo-2,4-dihydro-1H- |
| | [1,4]oxazepino[2,3-c]quinolin-10-yl]amino]pyrimidin-2-yl]-4-piperidyl]methyl]-1,4- |
| | diazepan-1-yl]-1-methyl-indazol-3-yl]piperidine-2,6-dione |
| 575 | 3-[6-[1-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-4-piperidyl]methyl]pyrrolidin-3- |
| | yl]oxy-1-methyl-indazol-3-yl]piperidine-2,6-dione |
| 575a | 3-(6-(((S*)-1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)pyrrolidin-3-yl)oxy)-1-methyl-1H-indazol-3-yl)piperidine-2,6-dione |
| 575b | 3-(6-(((R*)-1-((1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)piperidin-4- |
| | yl)methyl)pyrrolidin-3-yl)oxy)-1-methyl-1H-indazol-3-yl)piperidine-2,6-dione |
| 576 | 3-[6-[1-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-azaspiro[5.5]undecan-9- |
| | yl]methyl]-4-piperidyl]-1-methyl-indazol-3-yl]piperidine-2,6-dione |
| 576a | 3-(6-(1-((3-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3- |
| | azaspiro[5.5]undecan-9-yl)methyl)piperidin-4-yl)-1-methyl-1H-indazol-3-yl)piperidine-2,6- |
| | dione |
| 577 | 3-[6-[1-[[3-[4-[(2-cyclopropyl-3,3-difluoro-7-methyl-6-oxo-2,4-dihydro-1H- |
| | [1,4]oxazepino[2,3-c]quinolin-10-yl)amino]-5-fluoro-pyrimidin-2-yl]-3- |
| | azaspiro[5.5]undecan-9-yl]methyl]-4-piperidyl]-1-methyl-indazol-3-yl]piperidine-2,6-dione |
| 577a | 3-(6-(1-((3-(4-(((S)-2-cyclopropyl-3,3-difluoro-7-methyl-6-oxo-1,2,3,4,6,7-hexahydro- |
| | [1,4]oxazepino[2,3-c]quinolin-10-yl)amino)-5-fluoropyrimidin-2-yl)-3- |
| | azaspiro[5.5]undecan-9-yl)methyl)piperidin-4-yl)-1-methyl-1H-indazol-3-yl)piperidine-2,6- |
| | dione |
| 578 | 3-[6-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-fluoro-4- |
| | piperidyl]methyl]piperazin-1-yl]-1-methyl-indazol-3-yl]piperidine-2,6-dione |
| 578a | 3-(6-(4-(((3R*,4R*)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | fluoropiperidin-4-yl)methyl)piperazin-1-yl)-1-methyl-1H-indazol-3-yl)piperidine-2,6-dione |
| 578b | 3-(6-(4-(((3S*,4S*)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | fluoropiperidin-4-yl)methyl)piperazin-1-yl)-1-methyl-1H-indazol-3-yl)piperidine-2,6-dione |
| 579 | 3-[7-[4-[[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-fluoro-4- |
| | piperidyl]methyl]piperazin-1-yl]-1-methyl-indazol-3-yl]piperidine-2,6-dione |
| 579a | 3-(7-(4-(((3R*,4R*)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | fluoropiperidin-4-yl)methyl)piperazin-1-yl)-1-methyl-1H-indazol-3-yl)piperidine-2,6-dione |
| 579b | 3-(7-(4-(((3S*,4S*)-1-(5-chloro-4-(((S)-2-cyclopropyl-3,3-difluoro-7-methyl-6-oxo- |
| | 1,2,3,4,6,7-hexahydro-[1,4]oxazepino[2,3-c]quinolin-10-yl)amino)pyrimidin-2-yl)-3- |
| | fluoropiperidin-4-yl)methyl)piperazin-1-yl)-1-methyl-1H-indazol-3-yl)piperidine-2,6-dione |
| 581 | 3-[6-[1-[[3-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3-azaspiro[5.5]undecan-9- |
| | yl]methyl]-4-piperidyl]-7-fluoro-1-methyl-indazol-3-yl]piperidine-2,6-dione |
| 581a | 3-(6-(1-((3-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3- |
| | azaspiro[5.5]undecan-9-yl)methyl)piperidin-4-yl)-7-fluoro-1-methyl-1H-indazol-3- |
| | yl)piperidine-2,6-dione |
| 582 | 3-[7-[4-[1-[5-chloro-4-[(2-cyclopropyl-3,3-difluoro-7-methyl-6-oxo-2,4-dihydro-1H- |
| | [1,4]oxazepino[2,3-c]quinolin-10-yl)amino]pyrimidin-2-yl]-3,3-dimethyl-4- |
| | piperidyl]methyl]piperazin-1-yl]-1-methyl-indazol-3-yl]piperidine-2,6-dione |
| 582a | 3-(7-(4-(((R*)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3,3- |
| | dimethylpiperidin-4-yl)methyl)piperazin-1-yl)-1-methyl-1H-indazol-3-yl)piperidine-2,6- |
| | dione |
| 582b | 3-(7-(4-(((S*)-1-(5-chloro-4-(((S)-2-cyclopropyl-3,3-difluoro-7-methyl-6-oxo-1,2,3,4,6,7- |
| | hexahydro-[1,4]oxazepino[2,3-c]quinolin-10-yl)amino)pyrimidin-2-yl)-3,3- |
| | dimethylpiperidin-4-yl)methyl)piperazin-1-yl)-1-methyl-1H-indazol-3-yl)piperidine-2,6- |
| | dione |

Certain examples of Formula (I) compounds were synthesized using methods involving resolution of stereoisomeric mixture(s) (e.g., SFC separation of stereoisomers). In Table C1, the resolved stereogenic centers in these compounds are labelled with the “or1” and/or “or2” enhanced stereochemical notations. In some instances, the stereoisomeric resolutions were performed during the last step of the synthesis, thereby providing the individual stereoisomers of the Formula (I) compounds. Alternatively, in some other instances, the resolutions were performed on an intermediate or starting material, wherein each of the constituent stereoisomers of the intermediate or starting material could be separately subjected to the subsequent steps of the synthesis to provide the respective Formula (I) compounds as separate stereoisomers. Methods of resolution and correlation between resolved intermediates and Formula (I) compounds are disclosed in the examples herein and in Table P1. A person of ordinary skill in the art would understand that, under either approach for stereoisomeric resolution, stereoisomers having both (R)- and (S)-configurations at a resolved stereogenic center are provided. See Table C3, wherein Table C1 compounds whose stereoisomers contain the or1 and/or or2 stereochemical notations are provided in non-stereogenic form, followed by the respective stereoisomers having the (R)- and (S)-configurations.

TABLE C3

| No. | Compound Structure |
|---|---|
| 379 | 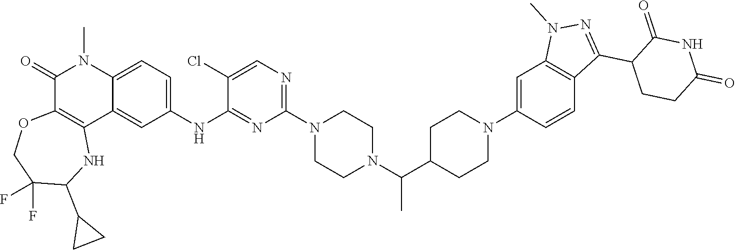 [SMILES: CC(C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)C(C4CC4)N3)n2)CC1] |
| | 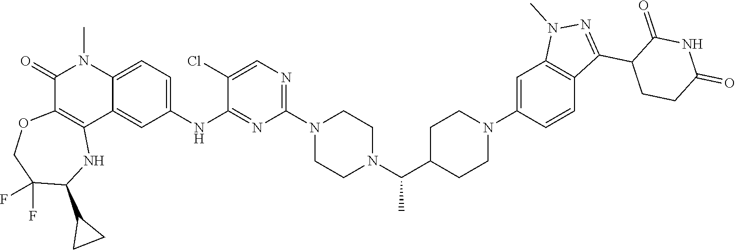 [SMILES: C[C@@H](C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)[C@H](C4CC4)N3)n2)CC1] |
| | 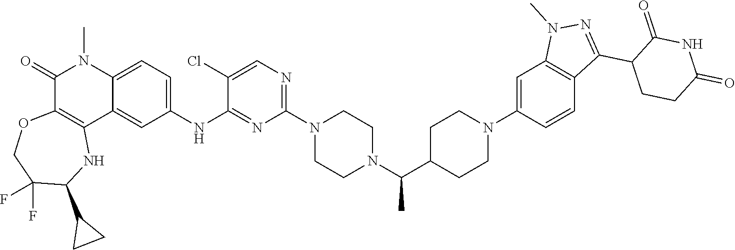 [SMILES: C[C@H](C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)[C@H](C4CC4)N3)n2)CC1] |
| 380 | 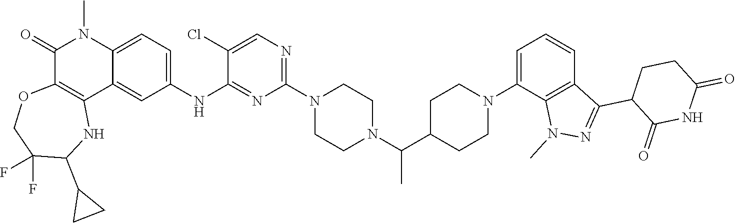 [SMILES: CC(C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)C(C4CC4)N3)n2)CC1] |
| | 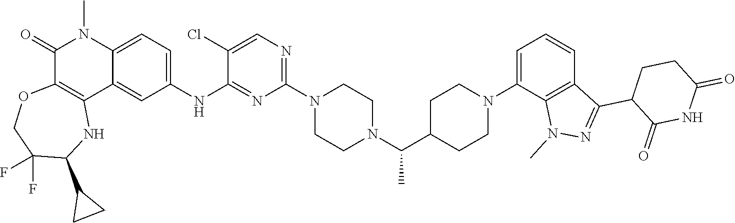 [SMILES: C[C@@H](C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)[C@H](C4CC4)N3)n2)CC1] |
| | 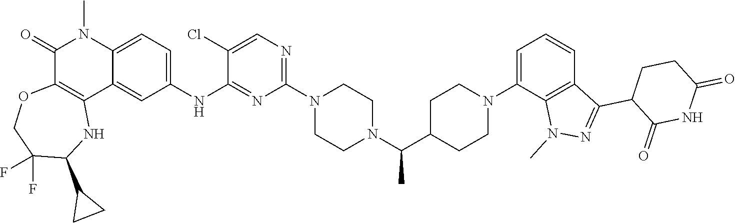 [SMILES: C[C@H](C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)[C@H](C4CC4)N3)n2)CC1] |
| 464 | 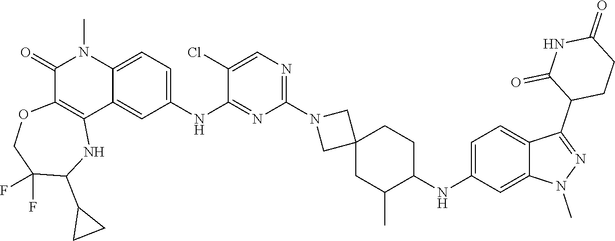 [SMILES: CC1CC2(CCC1Nc1ccc3c(C4CCC(=O)NC4=O)nn(C)c3c1)CN(c1ncc(Cl)c(Nc3ccc4c(c3)c3c(c(=O)n4C)OCC(F)(F)C(C4CC4)N3)n1)C2] |
| | 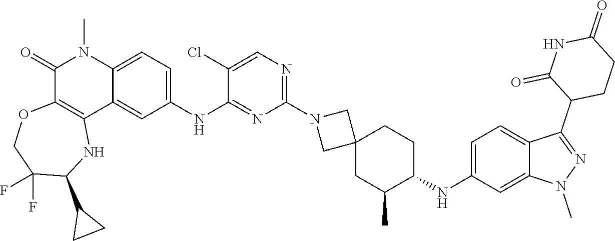 [SMILES: C[C@H]1CC2(CC[C@@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| | 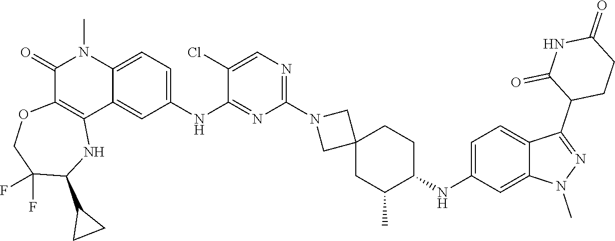 [SMILES: C[C@@H]1CC2(CC[C@@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| | 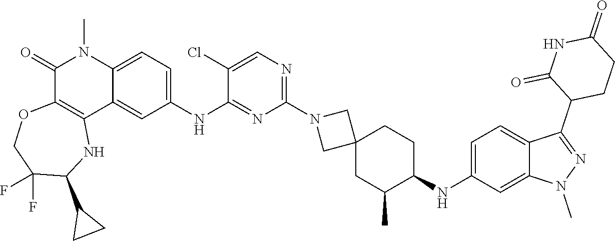 [SMILES: C[C@H]1CC2(CC[C@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| | 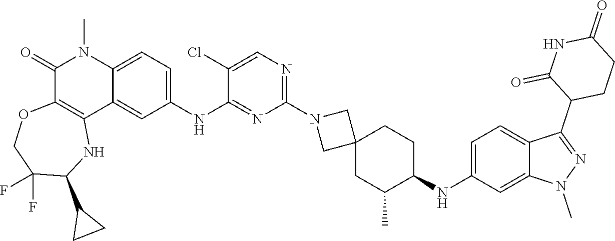 [SMILES: C[C@@H]1CC2(CC[C@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 465 | 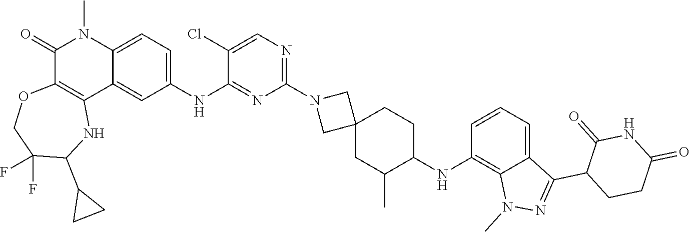 [SMILES: CC1CC2(CCC1Nc1cccc3c(C4CCC(=O)NC4=O)nn(C)c13)CN(c1ncc(Cl)c(Nc3ccc4c(c3)c3c(c(=O)n4C)OCC(F)(F)C(C4CC4)N3)n1)C2] |
| | 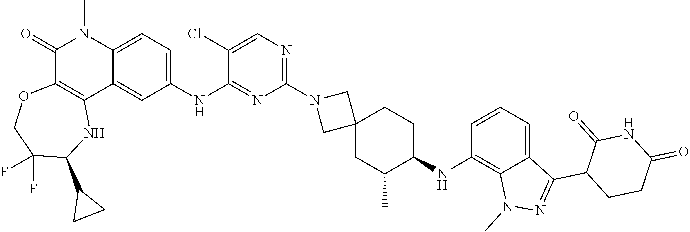 [SMILES: C[C@@H]1CC2(CC[C@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| | 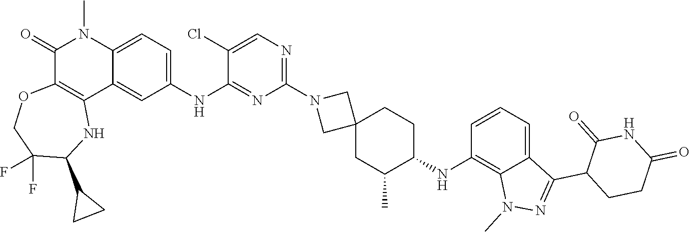 [SMILES: C[C@@H]1CC2(CC[C@@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| | 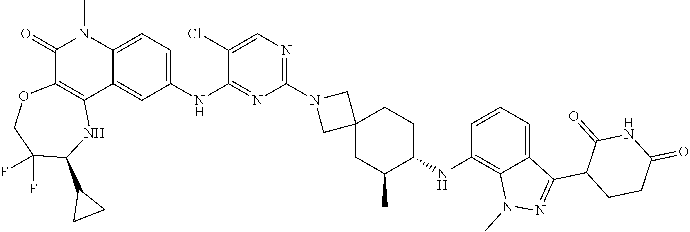 [SMILES: C[C@H]1CC2(CC[C@@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| | 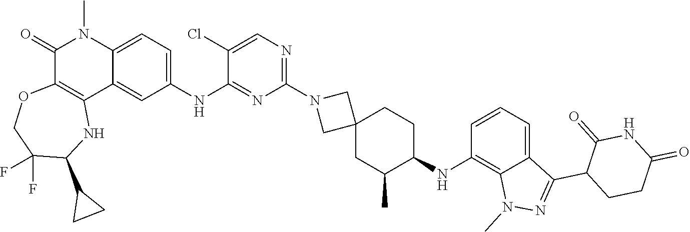 [SMILES: C[C@H]1CC2(CC[C@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2] |
| 492 | 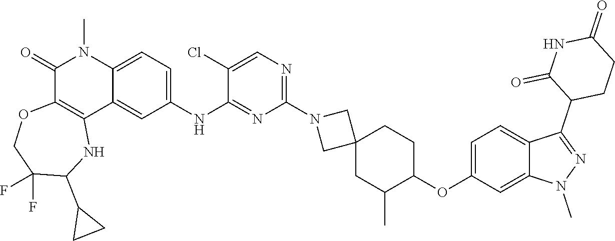 [SMILES: CC1CC2(CCC1Oc1ccc3c(C4CCC(=O)NC4=O)nn(C)c3c1)CN(c1ncc(Cl)c(Nc3ccc4c(c3)c3c(c(=O)n4C)OCC(F)(F)C(C4CC4)N3)n1)C2] |
| | 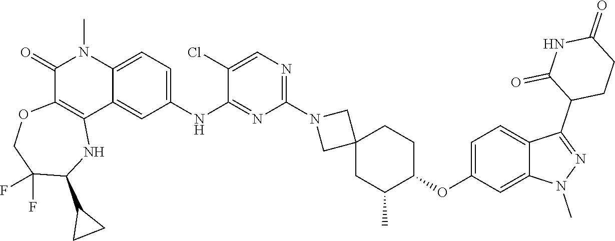 [SMILES: C[C@@H]1CC2(CC[C@@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| | 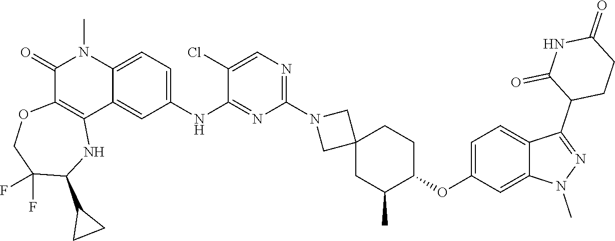 [SMILES: C[C@H]1CC2(CC[C@@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| | 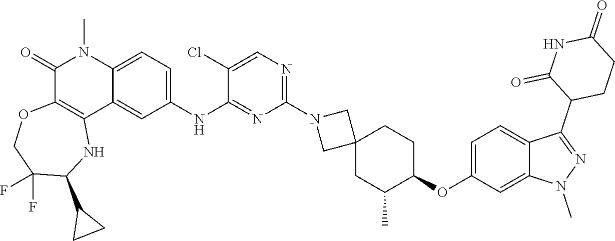 [SMILES: C[C@@H]1CC2(CC[C@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| | 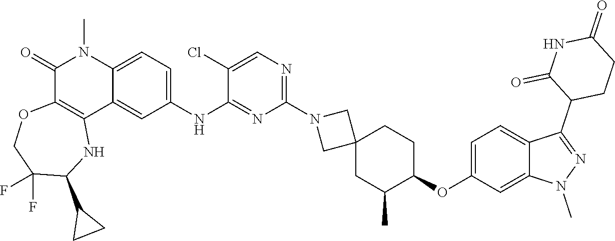 [SMILES: C[C@H]1CC2(CC[C@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2] |
| 499 | 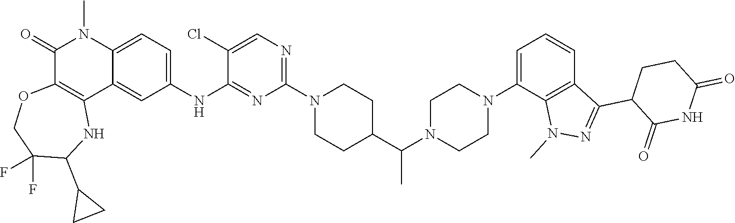 [SMILES: CC(C1CCN(c2ncc(Cl)c(Nc3ccc4c(c3)c3c(c(=O)n4C)OCC(F)(F)C(C4CC4)N3)n2)CC1)N1CCN(c2cccc3c(C4CCC(=O)NC4=O)nn(C)c23)CC1] |
| | 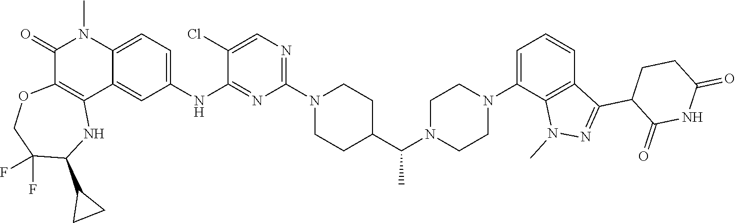 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCN(c2cccc3c(C4CCC(=O)NC4=O)nn(C)c23)CC1] |
| | 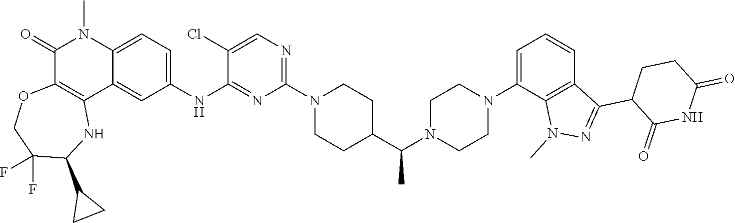 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCN(c2cccc3c(C4CCC(=O)NC4=O)nn(C)c23)CC1] |
| 500 | 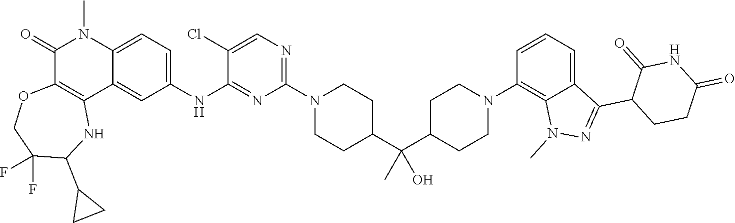 [SMILES: Cn1nc(C2CCC(=O)NC2=O)c2cccc(N3CCC(C(C)(O)C4CCN(c5ncc(Cl)c(Nc6ccc7c(c6)c6c(c(=O)n7C)OCC(F)(F)C(C7CC7)N6)n5)CC4)CC3)c21] |
| | 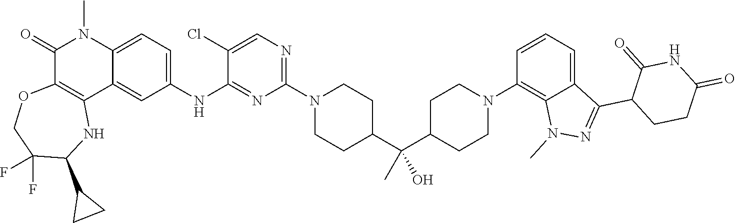 [SMILES: Cn1nc(C2CCC(=O)NC2=O)c2cccc(N3CCC([C@](C)(O)C4CCN(c5ncc(Cl)c(Nc6ccc7c(c6)c6c(c(=O)n7C)OCC(F)(F)[C@H](C7CC7)N6)n5)CC4)CC3)c21] |
| | 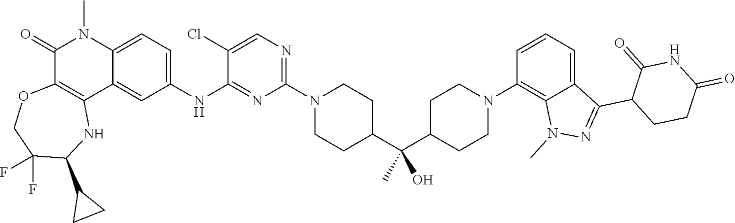 [SMILES: Cn1nc(C2CCC(=O)NC2=O)c2cccc(N3CCC([C@@](C)(O)C4CCN(c5ncc(Cl)c(Nc6ccc7c(c6)c6c(c(=O)n7C)OCC(F)(F)[C@H](C7CC7)N6)n5)CC4)CC3)c21] |
| 511 | 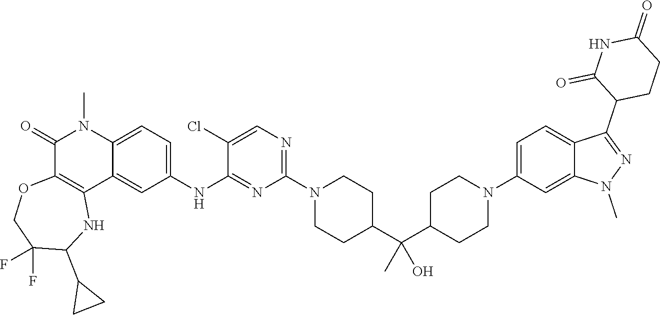 [SMILES: Cn1nc(C2CCC(=O)NC2=O)c2ccc(N3CCC(C(C)(O)C4CCN(c5ncc(Cl)c(Nc6ccc7c(c6)c6c(c(=O)n7C)OCC(F)(F)C(C7CC7)N6)n5)CC4)CC3)cc21] |
| | 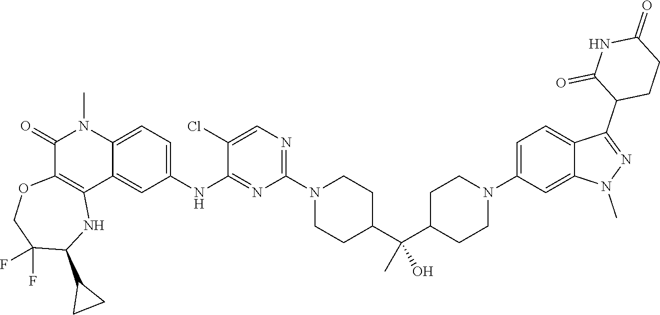 [SMILES: Cn1nc(C2CCC(=O)NC2=O)c2ccc(N3CCC([C@](C)(O)C4CCN(c5ncc(Cl)c(Nc6ccc7c(c6)c6c(c(=O)n7C)OCC(F)(F)[C@H](C7CC7)N6)n5)CC4)CC3)cc21] |
| | 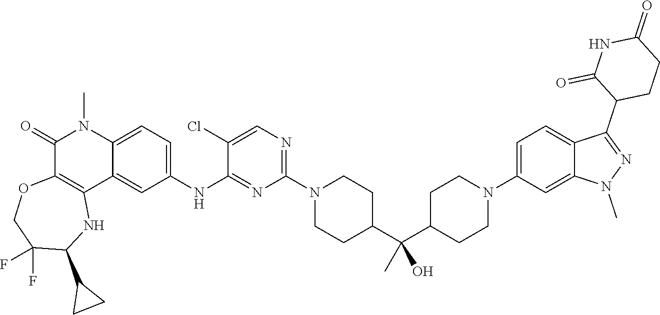 [SMILES: Cn1nc(C2CCC(=O)NC2=O)c2ccc(N3CCC([C@@](C)(O)C4CCN(c5ncc(Cl)c(Nc6ccc7c(c6)c6c(c(=O)n7C)OCC(F)(F)[C@H](C7CC7)N6)n5)CC4)CC3)cc21] |
| 522 | 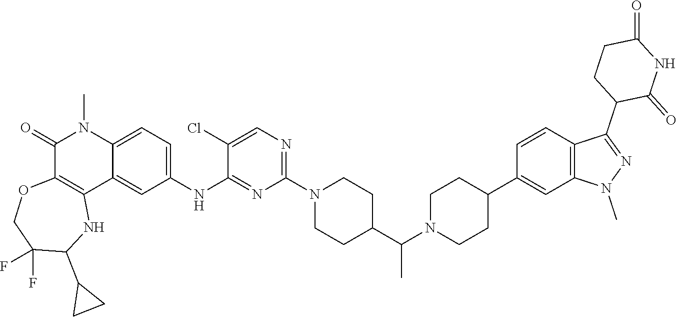 [SMILES: CC(C1CCN(c2ncc(Cl)c(Nc3ccc4c(c3)c3c(c(=O)n4C)OCC(F)(F)C(C4CC4)N3)n2)CC1)N1CCC(c2ccc3c(C4CCC(=O)NC4=O)nn(C)c3c2)CC1] |
| | 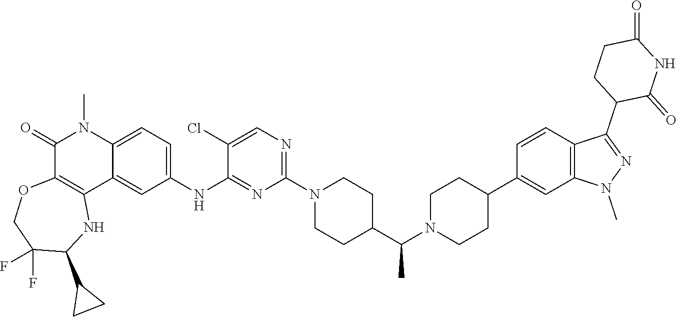 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCC(c2ccc3c(C4CCC(=O)NC4=O)nn(C)c3c2)CC1] |
| | 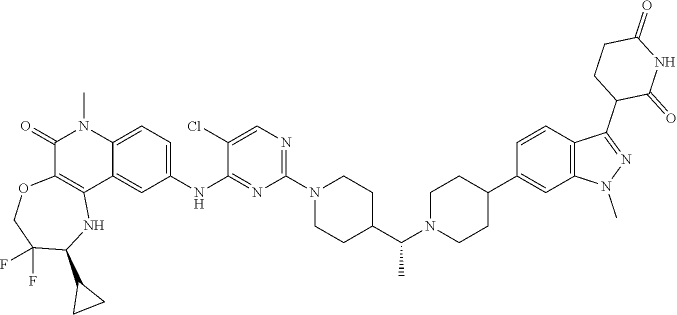 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCC(c2ccc3c(C4CCC(=O)NC4=O)nn(C)c3c2)CC1] |
| 523 | 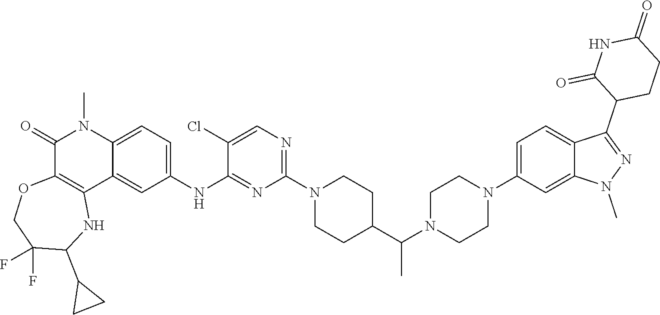 [SMILES: CC(C1CCN(c2ncc(Cl)c(Nc3ccc4c(c3)c3c(c(=O)n4C)OCC(F)(F)C(C4CC4)N3)n2)CC1)N1CCN(c2ccc3c(C4CCC(=O)NC4=O)nn(C)c3c2)CC1] |
| | 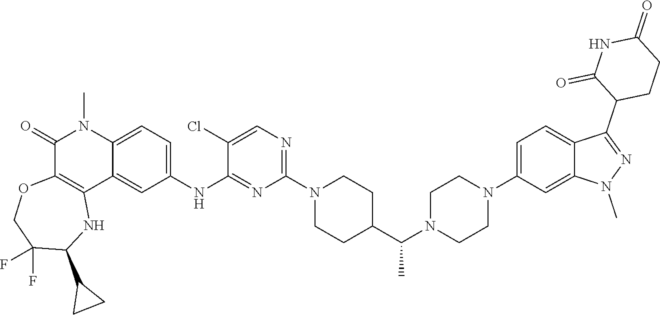 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCN(c2ccc3c(C4CCC(=O)NC4=O)nn(C)c3c2)CC1] |
| | 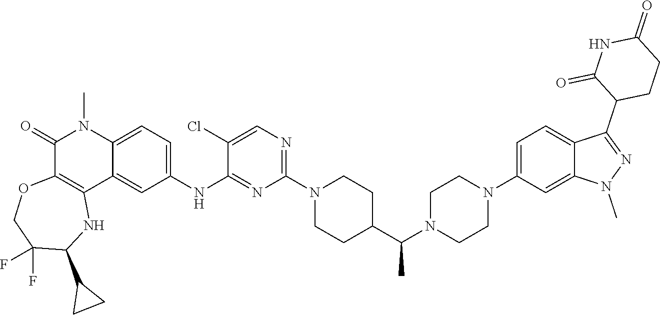 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCN(c2ccc3c(C4CCC(=O)NC4=O)nn(C)c3c2)CC1] |
| 534 | 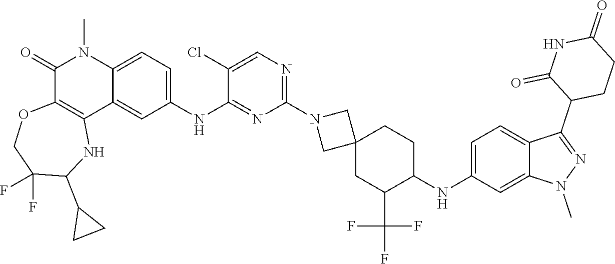 [SMILES: Cn1nc(C2CCC(=O)NC2=O)c2ccc(NC3CCC4(CC3C(F)(F)F)CN(c3ncc(Cl)c(Nc5ccc6c(c5)c5c(c(=O)n6C)OCC(F)(F)C(C6CC6)N5)n3)C4)cc21] |
| | 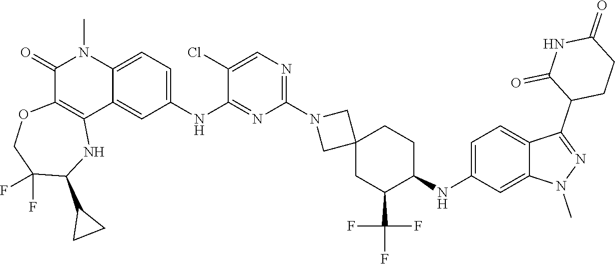 [SMILES: Cn1nc(C2CCC(=O)NC2=O)c2ccc(N[C@@H]3CCC4(C[C@@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| | 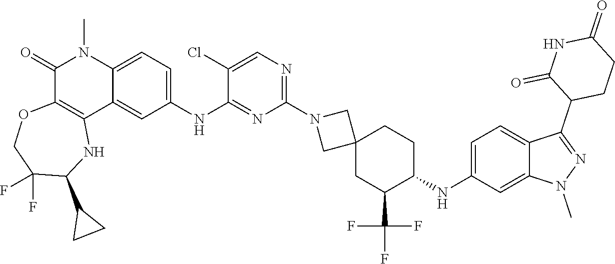 [SMILES: Cn1nc(C2CCC(=O)NC2=O)c2ccc(N[C@H]3CCC4(C[C@@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| | 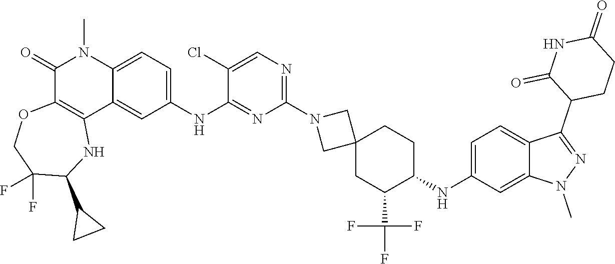 [SMILES: Cn1nc(C2CCC(=O)NC2=O)c2ccc(N[C@H]3CCC4(C[C@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| | 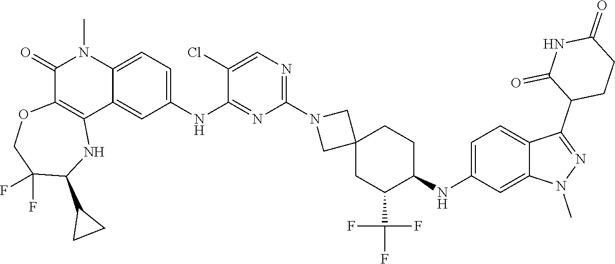 [SMILES: Cn1nc(C2CCC(=O)NC2=O)c2ccc(N[C@@H]3CCC4(C[C@H]3C(F)(F)F)CN(c3ncc(Cl)c(Nc5ccc6c(c5)c5c(c(=O)n6C)OCC(F)(F)[C@H](C6CC6)N5)n3)C4)cc21] |
| 568 | 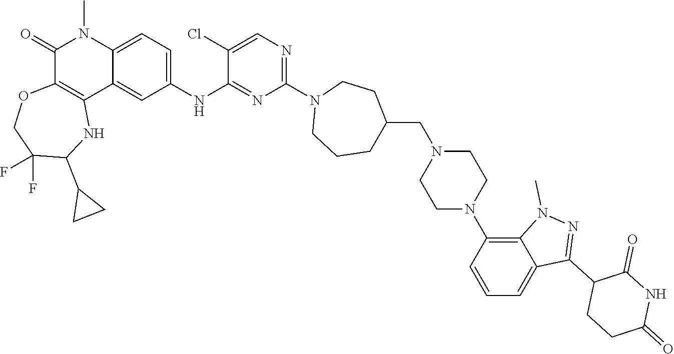 [SMILES: Cn1nc(C2CCC(=O)NC2=O)c2cccc(N3CCN(CC4CCCN(c5ncc(Cl)c(Nc6ccc7c(c6)c6c(c(=O)n7C)OCC(F)(F)C(C7CC7)N6)n5)CC4)CC3)c21] |
| | 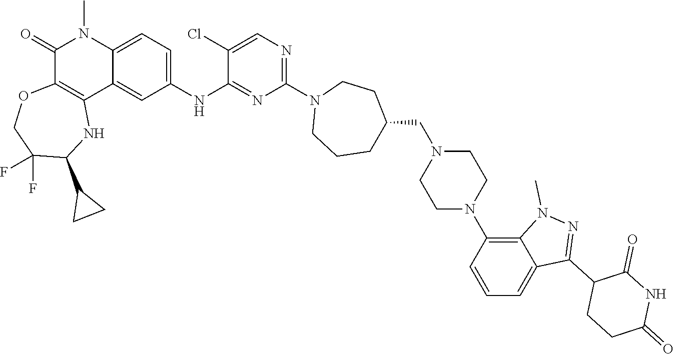 [SMILES: Cn1nc(C2CCC(=O)NC2=O)c2cccc(N3CCN(C[C@@H]4CCCN(c5ncc(Cl)c(Nc6ccc7c(c6)c6c(c(=O)n7C)OCC(F)(F)[C@H](C7CC7)N6)n5)CC4)CC3)c21] |
| | 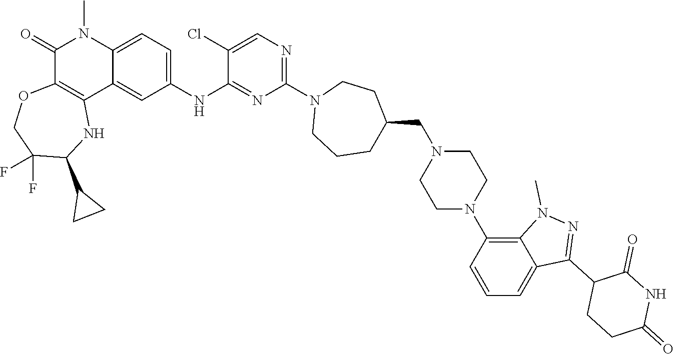 [SMILES: Cn1nc(C2CCC(=O)NC2=O)c2cccc(N3CCN(C[C@H]4CCCN(c5ncc(Cl)c(Nc6ccc7c(c6)c6c(c(=O)n7C)OCC(F)(F)[C@H](C7CC7)N6)n5)CC4)CC3)c21] |
| 575 | 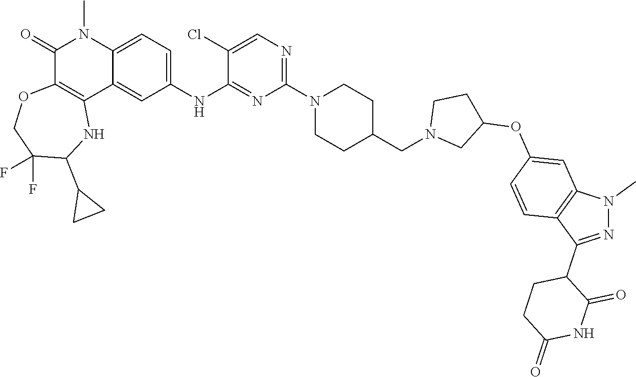 [SMILES: Cn1nc(C2CCC(=O)NC2=O)c2ccc(OC3CCN(CC4CCN(c5ncc(Cl)c(Nc6ccc7c(c6)c6c(c(=O)n7C)OCC(F)(F)C(C7CC7)N6)n5)CC4)C3)cc21] |
| | 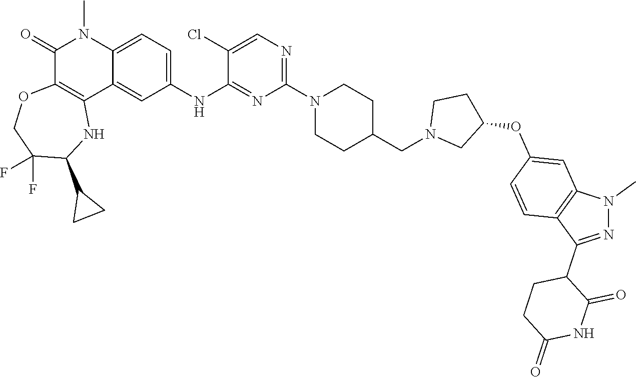 [SMILES: Cn1nc(C2CCC(=O)NC2=O)c2ccc(O[C@H]3CCN(CC4CCN(c5ncc(Cl)c(Nc6ccc7c(c6)c6c(c(=O)n7C)OCC(F)(F)[C@H](C7CC7)N6)n5)CC4)C3)cc21] |
| | 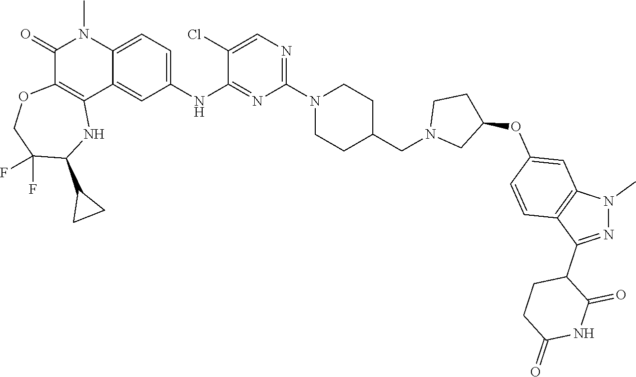 [SMILES: Cn1nc(C2CCC(=O)NC2=O)c2ccc(O[C@@H]3CCN(CC4CCN(c5ncc(Cl)c(Nc6ccc7c(c6)c6c(c(=O)n7C)OCC(F)(F)[C@H](C7CC7)N6)n5)CC4)C3)cc21] |
| 578 | 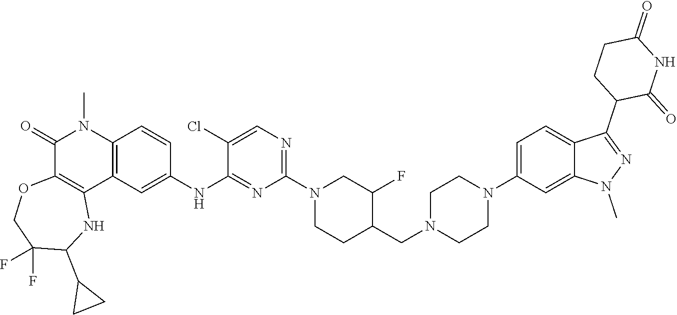 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)C(C7CC7)N6)n5)CC4F)CC3)cc21] |
| | 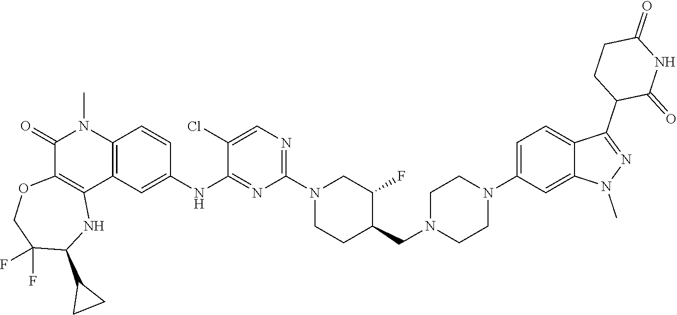 [SMILES: Cn1nc(C2CCC(=O)NC2=O)c2ccc(N3CCN(C[C@H]4CCN(c5ncc(Cl)c(Nc6ccc7c(c6)c6c(c(=O)n7C)OCC(F)(F)[C@H](C7CC7)N6)n5)C[C@@H]4F)CC3)cc21] |
| | 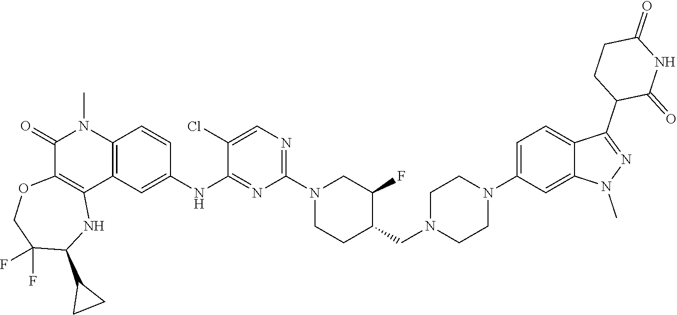 [SMILES: Cn1nc(C2CCC(=O)NC2=O)c2ccc(N3CCN(C[C@@H]4CCN(c5ncc(Cl)c(Nc6ccc7c(c6)c6c(c(=O)n7C)OCC(F)(F)[C@H](C7CC7)N6)n5)C[C@H]4F)CC3)cc21] |
| 579 | 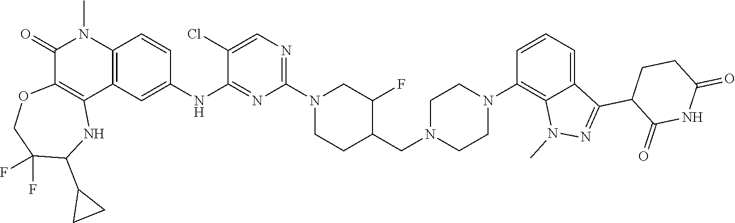 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)C(C7CC7)N6)n5)CC4F)CC3)c21] |
| | 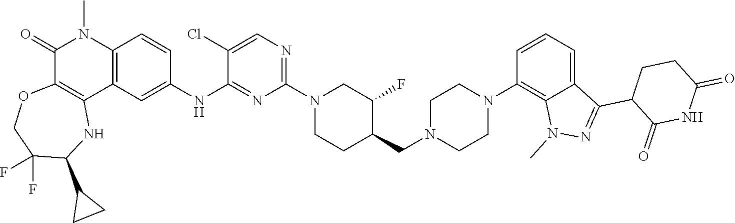 [SMILES: Cn1nc(C2CCC(=O)NC2=O)c2cccc(N3CCN(C[C@H]4CCN(c5ncc(Cl)c(Nc6ccc7c(c6)c6c(c(=O)n7C)OCC(F)(F)[C@H](C7CC7)N6)n5)C[C@@H]4F)CC3)c21] |
| | 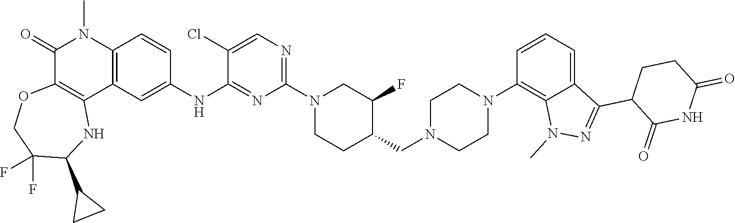 [SMILES: Cn1nc(C2CCC(=O)NC2=O)c2cccc(N3CCN(C[C@@H]4CCN(c5ncc(Cl)c(Nc6ccc7c(c6)c6c(c(=O)n7C)OCC(F)(F)[C@H](C7CC7)N6)n5)C[C@H]4F)CC3)c21] |
| 582 | 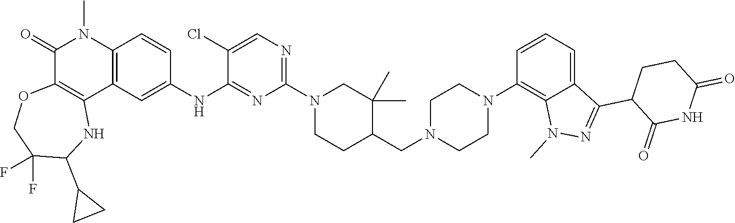 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)C(C7CC7)N6)n5)CC4(C)C)CC3)c21] |
| | 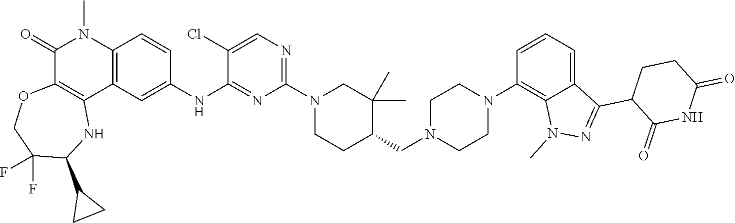 [SMILES: Cn1nc(C2CCC(=O)NC2=O)c2cccc(N3CCN(C[C@@H]4CCN(c5ncc(Cl)c(Nc6ccc7c(c6)c6c(c(=O)n7C)OCC(F)(F)[C@H](C7CC7)N6)n5)CC4(C)C)CC3)c21] |
| | 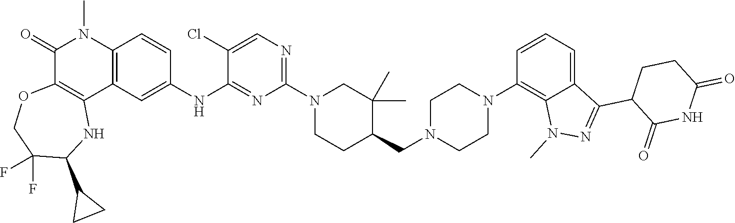 [SMILES: Cn1nc(C2CCC(=O)NC2=O)c2cccc(N3CCN(C[C@H]4CCN(c5ncc(Cl)c(Nc6ccc7c(c6)c6c(c(=O)n7C)OCC(F)(F)[C@H](C7CC7)N6)n5)CC4(C)C)CC3)c21] |

Exemplary compounds of Formula (I) also include those depicted in Table C1 of U.S. Provisional Application Ser. No. 63/414,349, filed Oct. 7, 2022; Table C1 of U.S. Provisional Application Ser. No. 63/414,418, filed Oct. 7, 2022; Table C1 of U.S. Provisional Application Ser. No. 63/420,398, filed Oct. 28, 2022; Table C1 of U.S. Provisional Application Ser. No. 63/444,792, filed Feb. 10, 2023; Table C1 of U.S. Provisional Application Ser. No. 63/497,061, filed Apr. 19, 2023; and Table C1 of U.S. Provisional Application Ser. No. 63/501,080, filed May 9, 2023; or pharmaceutically acceptable salts thereof, wherein each Table C1 is incorporated herein by reference in its entirety.

Also provided herein are compounds of Formula (II):

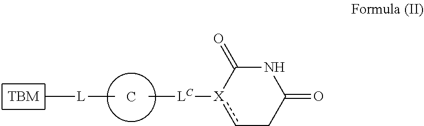

or a pharmaceutically acceptable salt thereof, wherein:
TBM is selected from the group consisting of (T1) and (T2):

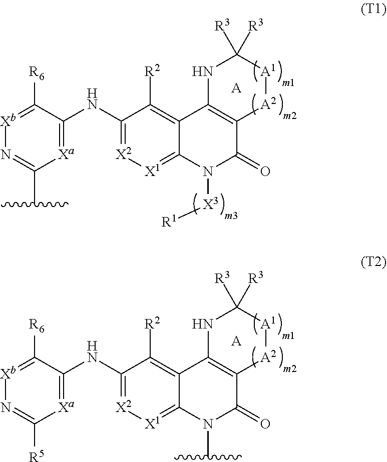

X¹ and X² are independently selected from the group consisting of: N and CR²;
each R² is independently selected from the group consisting of: H, halo, cyano, C1-3 alkyl, C₁₋₃ haloalkyl, C₁₋₃ alkoxy, C₁₋₃ haloalkoxy, —OH, and —NR^{d}R^{e};
m3 is 0, 1, 2, or 3;
each X³ is independently selected from the group consisting of: —O—, —NR^{f}—, —C(═O)—, and C₁₋₃ alkylene optionally substituted with 1-3 R^{e};
R¹ is selected from the group consisting of: H, halo, cyano, and R^{b1};
provided that the N—(X³)ₘ₃—R¹ moiety does not contain any O—O, N—O, or N-halo bonds;
m1 is 1, 2, or 3, and m2 is 0 or 1, wherein m1+m2 is 2 or 3;
each A¹ is independently selected from the group consisting of: CH₂, CHR⁴, CR⁴R⁴, O, —NR^{d}—, C(═O), and S(O)₀₋₂;
A² is selected from the group consisting of: O, —NR^{d}—, C(═O), and S(O)₀₋₂;
provided that Ring A does not contain any O—O, N—O, S—O, or S(O)₀—N bonds;
each R³ is independently selected from the group consisting of:
a) H;
b) C₁₋₆ alkyl optionally substituted with 1-6 R^{c};
c) C(O)OR^{f} or C(O)N(R)₂;
d) cyano; and
e) R^{b};
each R⁴ is independently selected from the group consisting of: H, R^{a}, and R^{b}; or
one pair of R³ or one pair of R⁴, taken together with the ring carbon atom(s) connecting them, form a C₃₋₆ cycloalkyl ring or a 4-6 membered heterocyclyl ring, wherein the C₃₋₆ cycloalkyl ring or the 4-6 membered heterocyclyl ring is optionally substituted with 1-3 R^{a};
X^{a} is selected from the group consisting of: N, CH, and CF;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
R⁵ is selected from the group consisting of:
a) H;
b) halo;
c) cyano;
d) NO₂;
e) C₁₋₆ alkyl, C₂₋₆ alkenyl, or C₂₋₆ alkynyl, each optionally substituted with 1-6 R^{e} and further optionally substituted with R^{b1};
f) C₁₋₆ alkoxy optionally substituted with 1-6 R^{e} and further optionally substituted with R^{b1}; and
g) -L⁵¹-R⁵¹;
L⁵¹ is selected from the group consisting of: a bond, O, S(O)₀₋₂, C(O), C(O)O*, OC(O)*, C(O)N(R^{f})*, N(R^{f})C(O)*, N(R^{f})C(O)N(R^{f}), N(R^{f})C(O)O*, OC(O)N(R^{f})*, S(O)₂N(R^{f})*, N(R^{f})SO₂*, and —N(R^{d})—(C₀₋₂ alkylene)-*, wherein * represents the point of attachment to R⁵¹, and the C₀₋₂ alkylene portion of —N(R^{d})—(C₀₋₂ alkylene)-* is optionally substituted with 1-3 substituents independently selected from the group consisting of: C₁₋₃ alkyl and halo;
R⁵¹ is selected from the group consisting of:
a) H;
b) C₁₋₆ alkyl, C₂₋₆ alkenyl, or C₂₋₆ alkynyl, each optionally substituted with 1-6 R^{e} and further optionally substituted with R^{b1}; and
c) C₃₋₁₀ cycloalkyl, 4-12 membered heterocyclyl, C₆₋₁₀ aryl, or 5-10 membered heteroaryl, each of which is optionally substituted with 1-6 substituents independently selected from the group consisting of: R^{a} and R^{b};
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1} L^{A3}, and L^{A4}, provided that 1-3 occurrences of L^{A} is L^{A4};

### (BB)

n1 is an integer from 0 to 20; and
each L^{A} is independently selected from the group consisting of: L^{A}1 and L^{A3}; each L^{A}1 is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, N—N, N—S(O)o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{c};
Ring C is selected from the group consisting of:

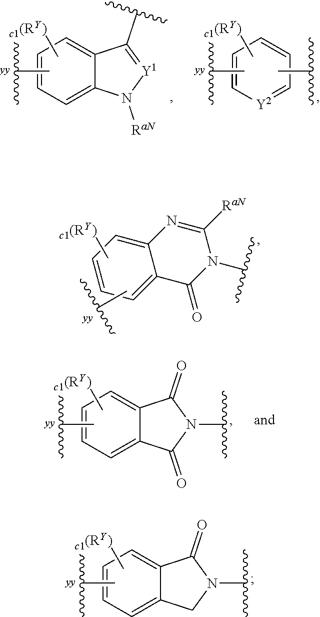

c1 is 0, 1, 2, or 3;
each R^{Y} is independently selected from the group consisting of: R^{a} and R^{b};
R^{aN} is H or C₁₋₆ alkyl optionally substituted with 1-3 R^{e};
Y¹ and Y² are independently N, CH, or CR^{Y};
yy represents the point of attachment to L;
X is CH, C, or N;
the is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -Le- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)C₁₋₆ haloalkyl;
(i) C(═O)OH;
(j) C(═O)OC₁₋₆ alkyl;
(k) C(═O)OC₁₋₆ haloalkyl;
(l) C(═O)N(R^{f})₂;
(m) S(O)₀₋₂(C₁₋₆ alkyl);
(n) S(O)₀₋₂(C₁₋₆ haloalkyl);
(o) S(O)₁₋₂N(Rf)₂; and
(p) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(Rf)₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In Formula (II), L, Ring C, L^{C}, and X can be as defined herein for Formula (I).

Exemplary compounds of Formula (II) include those depicted in Table C1 of U.S. Provisional Application Ser. No. 63/349,415, filed Jun. 6, 2022; Table C1 of U.S. Provisional Application Ser. No. 63/349,420, filed Jun. 6, 2022; Table C1 of U.S. Provisional Application Ser. No. 63/356,388, filed Jun. 28, 2022; Table C1 of U.S. Provisional Application Ser. No. 63/356,390, filed Jun. 28, 2022; Table C1 of U.S. Provisional Application Ser. No. 63/414,409, filed Oct. 7, 2022; Table C1 of U.S. Provisional Application Ser. No. 63/414,362, filed Oct. 7, 2022; Table C1 of U.S. Provisional Application Ser. No. 63/420,385, filed Oct. 28, 2022; and Table C1 of U.S. Provisional Application Ser. No. 63/444,769, filed Feb. 10, 2023; or pharmaceutically acceptable salts thereof, wherein each Table C1 is incorporated herein by reference in its entirety.

In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, reduce cell viability in a cell line expressing a BCL6 protein with an EC₅₀ of less than 1 μM (e.g., less than 750 nM, less than 500 nM, or less than 200 nM). In some embodiments, the compounds reduce cell viability in a cell line expressing the BCL6 protein with an EC₅₀ of less than 200 nM (e.g., less than 150 nM, less than 200 nM, less than 100 nM, less than 10 nM, less than 1 nM). For example, the compounds can reduce cell viability in a cell line expressing the BCL6 protein with an EC₅₀ of about 0.1 nM to about 100 nM, about 0.1 nM to about 50 nM, about 1 nM to about 50 nM, about 1 nM to about 20 nM, or about 0.1 nM to about 1 nM.

In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a DC₅₀ of less than 1 μM (e.g., less than 750 nM, less than 500 nM, or less than 200 nM). In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a DC₅₀ of less than 200 nM (e.g., less than 150 nM, less than 200 nM, less than 100 nM, less than 10 nM, less than 1 nM). For example, the compounds can induce degradation of a BCL6 protein in a cell line expressing the BCL6 protein with a DC₅₀ of about 0.1 nM to about 100 nM, about 0.1 nM to about 50 nM, about 1 nM to about 50 nM, about 1 nM to about 20 nM, or about 0.1 nM to about 1 nM.

In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a Yₘᵢₙ of less than 70% (e.g., less than 50%, less than 30%, less than 20%, or less than 10%). In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a Yₘᵢₙ of less than 50% (e.g., less than 40%, less than 30%, less than 20%, less than 10%, or less than 5%). In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a Yₘᵢₙ of less than 30% (e.g., less than 25%, less than 20%, less than 15%, less than 10%, or less than 5%). For example, the compounds can induce degradation of a BCL6 protein in a cell line expressing the BCL6 protein with a Yₘᵢₙ of about 1% to about 70% (e.g., about 5% to about 50% or about 10% to about 30%).

Also provided herein is a BCL6 protein non-covalently bound with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Also provided herein is a ternary complex comprising a BCL6 protein, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and a CRBN protein or a portion thereof.

### Chemical Definitions

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively.

For avoidance of doubt, in “cycloalkylene” and “heterocyclylene”, the two radicals can be on the same ring carbon atom (e.g., a geminal diradical such as

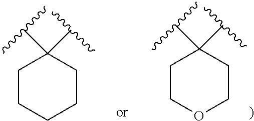 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

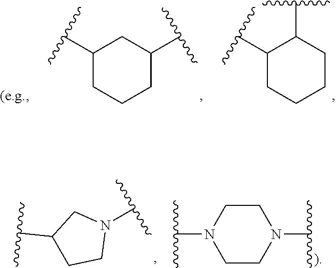 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall.

Examples of partially unsaturated cycloalkyl include, without limitation, cyclopentenyl, cyclohexenyl, cycloheptenyl, and cyclooctenyl. Cycloalkyl may include multiple fused and/or bridged rings. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Cycloalkyl also includes spirocyclic rings (e.g., spirocyclic bicycle wherein two rings are connected through just one atom).

Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, S (inclusive of oxidized forms such as:

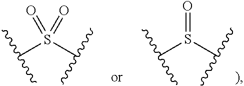

and P (inclusive of oxidized forms such as:

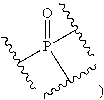 [SMILES: *P(*)(*)=O]

(e.g., N, O, and S (inclusive of oxidized forms such as:

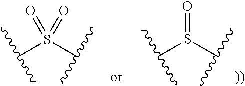

and at least one ring in the system is aromatic (but does not have to be a ring which contains a heteroatom, e.g. tetrahydroisoquinolinyl, e.g., tetrahydroquinolinyl). In some embodiments, heteroaryl groups contain 1-4 (e.g., 1, 2, or 3) ring heteroatoms each independently selected from the group consisting of N, O, and S (inclusive of oxidized forms such as:

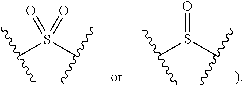

Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

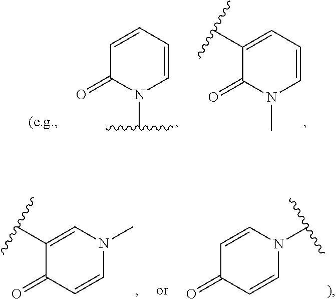

pyrimidone

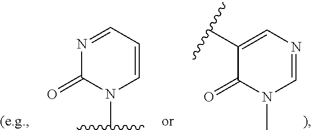

pyridazinone

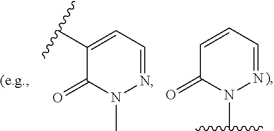

pyrazinone

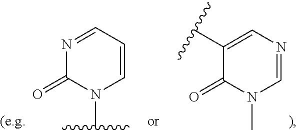

and imidazolone

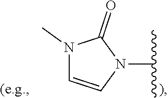 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, said heteroatoms selected from O, N, S (inclusive of oxidized forms such as:

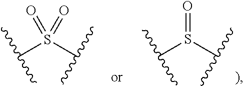

and P (inclusive of oxidized forms such as:

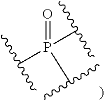 [SMILES: *P(*)(*)=O]

(e.g., N, O, and S (inclusive of oxidized forms such as:

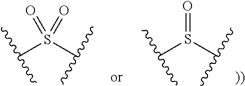

(e.g., carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl.

Heterocyclyl may include multiple fused and bridged rings. Non-limiting examples of fused/bridged heteorocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrolyl, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Heterocyclyl also includes spirocyclic rings (e.g., spirocyclic bicycle wherein two rings are connected through just one atom).

Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

A nitrogen-containing heterocyclyl as used herein refers to a heterocyclyl having 1-2 ring nitrogen atoms and 0-2 additional ring heteroatoms selected from the group consisting of O and S (inclusive of oxidized such as:

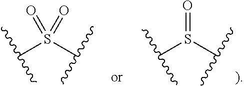

The nitrogen-containing heterocyclyl can be monocyclic, bicyclic, or polycyclic as defined elsewhere herein. Examples of monocyclic nitrogen-containing heterocyclyl include azetidinyl, pyrrolidinyl, piperidinyl, piperazinyl, morpholinyl, and the like. Examples of bicyclic nitrogen-containing heterocyclyl include 7-azaspiro[3.5]nonyl, 1,7-diazaspiro[4.5]decyl, 3-oxa-7,9-diazabicyclo[3.3.1]nonanyl, 2,6-diazaspiro[3.3]heptanyl, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

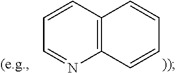

(ii) a single ring atom (spiro-fused ring systems)

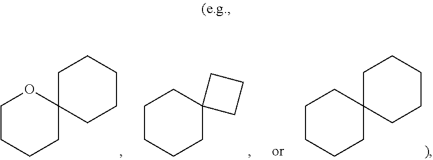

or (iii) a contiguous array of ring atoms (bridged ring systems having all bridge lengths>0)

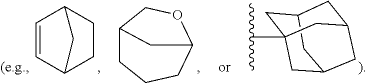

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

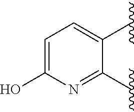 [SMILES: *c1ccc(O)nc1*]

encompasses the tautomeric form containing the moiety:

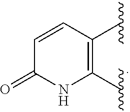 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

### Methods of Treatment

### Indications

Provided herein are methods for inducing degradation of a BCL6 protein. For example, provided herein are compounds capable of inducing degradation of a BCL6 protein useful for treating or preventing cancers. Exemplary compounds that bind to BCL6 are described in, e.g., Cerchietti, Leandro C., et al. Cancer Cell 17.4 (2010): 400-411; Cardenas, Mariano G., et al. The Journal of Clinical Investigation 126(9) (2016): 3351-3362; Kerres, Nina, et al., Cell Reports 20.12 (2017): 2860-2875; Yasui, Takeshi, et al., Bioorganic & Medicinal Chemistry 25.17 (2017): 4876-4886; Kamada, Yusuke, et al., Journal of Medicinal Chemistry 60.10 (2017): 4358-4368; McCoull, William, et al., ACS Chemical Biology 13.11 (2018): 3131-3141; Guo, Weikai, et al. Journal of Medicinal Chemistry 63.2 (2020): 676-695; Teng, Mingxing, et al., ACS Medicinal Chemistry Letters 11.6 (2020): 1269-1273; Pearce, Andrew C., et al., Journal of Biological Chemistry 297.2 (2021); Ding, Shu, Yu Rao, and Qianjin Lu, Cellular & Molecular Immunology (2022): 1-3; Xing, Y. et al., Cancer Letters (2022), doi: 10.1016/j.canlet.2021.12.035; Huckvale, R. et al., Journal of Medicinal Chemistry (2022), doi: 10.1021/acs.jmedchem.1c02175; Davis, O. et al., Journal of Medicinal Chemistry (2022), doi: 10.1021/acs.jmedchem.1c02174; International Publication Nos. WO 2008/066887; WO 2010/008436; WO 2014/204859; WO 2018/215798; WO 2018/215801; WO 2018/219281; WO 2019/119138; WO 2019/119144; WO 2019/119145; WO 2019/153080; WO 2019/197842; WO 2020/104820; WO 2021/074620; WO 2021/077010, and WO 2022/221673.

Potency of degradation by a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein can be determined by DC₅₀ value. As used herein, DC₅₀ refers to the concentration of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II) that results in a 50% decrease in the concentration of a protein (e.g., BCL6 protein) in a cell compared to the concentration of the protein before the cell is contacted with the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or compared to the concentration of the protein in a cell not contacted with the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II).

A compound with a lower DC₅₀ value, as determined under substantially similar conditions, is a more potent inducer of degradation relative to a compound with a higher DC₅₀ value. In some embodiments, a DC₅₀ value can be determined (e.g., using HiBiT detection) in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

Potency of degradation by a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein can be determined by EC₅₀ value. As used herein, EC₅₀ refers to the concentration of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II) that results in a 50% decrease in the concentration of a protein (e.g., BCL6 protein) relative to the trough concentration of the protein in a cell, when compared to the concentration of the protein before the cell is contacted with the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or compared to the concentration of the protein in a cell not contacted with the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II). A compound with a lower EC₅₀ value, as determined under substantially similar conditions, is a more potent inducer of degradation relative to a compound with a higher EC₅₀ value. In some embodiments, a EC₅₀ value can be determined (e.g., using HiBiT detection) in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950,WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

Potency of degradation by a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein can be determined by a Yₘᵢₙ value. As used herein, Yₘᵢₙ refers to the ratio of trough concentration of a protein (e.g., BCL6 protein) in a cell compared to the concentration of the protein before the cell is contacted with the compound of Formula (I), or compared to the concentration of the protein in a cell not contacted with the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), expressed as a percentage. As used herein, Dₘₐₓ is 1-Yₘᵢₙ. Yₘᵢₙ can be measured by a HiBiT assay as described in Example B1. A compound with a lower Yₘᵢₙ value, as determined under substantially similar conditions, is a more potent inducer of degradation relative to a compound with a higher Yₘᵢₙ value. In some embodiments, a Yₘᵢₙ value can determined (e.g., using HiBiT detection), in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 70% (e.g., less than 50% or less than 30%) in a HiBiT based degradation assay (e.g., an assay as described in Example B1). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 50% (e.g., less than 30%) in a HiBiT based degradation assay. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (T-b-1) or (I-b-2))) or Formula (II), or a pharmaceutically acceptable salt thereof, exhibits a Yₘᵢₙ of less than 30% in a HiBiT based degradation assay.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 70% (e.g., less than 50% or less than 30%) in the assay described in Example B1. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 50% (e.g., less than 30%) in the assay described in Example B1. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 30% in the assay described in Example B1.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 70% (e.g., about 0% to about 50%, about 30% to about 50%, or about 0% to about 30%) in a HiBiT based degradation assay (e.g., an assay as described in Example B1). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 50% (e.g., about 30% to about 50% or about 0% to about 30%) in a HiBiT based degradation assay. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 30% in a HiBiT based degradation assay.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 70% (e.g., about 0% to about 50%, about 30% to about 50%, or about 0% to about 30%) in the assay described in Example B1. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 50% (e.g., about 30% to about 50% or about 0% to about 30%) in the assay described in Example B1. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 30% in the assay described in Example B1.

It will be understood that the effect of protein degradation typically increases over time, though the appearance of degradation (e.g., as expressed by the percentage degradation compared to a control, or the parameters Yₘᵢₙ, DC₅₀, and/or Dₘₐₓ) is affected by the resynthesis rate of the protein. It is common in the art to examine degradation after a specified period of time, such as 6 hours, 12 hours, 18 hours, 1 day, 2 days, 3 days, 5 days, 10 days, or more. For example, degradation can be expressed as the percent degradation after 24 hours.

Exemplary assays for validating the degradation-inducing mechanism of a compound as provided herein are known in the art and are described, for example, in Wu, et al. Nature Structural & Molecular Biology 27.7 (2020): 605-614.

Degradation assays can be used to quantify both on- and off-target degradation-inducing effects of compounds, such as those provided herein. Exemplary assays include, quantitative immunoblotting, other immunoassays (e.g., MesoScale Discovery (MSD) immunoassays), homogenous time resolved florescence (HTRF), and HiBiT. In some embodiments, cells can be contacted with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, incubated, and then the lysate can be prepared for gel electrophoresis (e.g., SDS-PAGE), followed by immunoblotting and quantification compared to a control (e.g., a DMSO-treated control). As another example, a cell line can be engineered to express a HiBiT-tagged BCL6 protein, and the amount of fluorescence observed when the complementary LgBiT peptide is added can be compared between cells treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and a control (e.g., a DMSO-treated control). See, for instance, Example B1. In some embodiments, off-target degradation inducing effects can be assessed for the proteins Eukaryotic peptide chain release factor GTP-binding subunit ERF3A (GSPT1), Ikaros (IKZF1), Helios (IKZF2), Aiolos (IKZF3), and/or casein kinase I isoform alpha (CK1α). See also, e.g., International Publication Nos. WO 2018/215798; WO 2018/215801; WO 2020/104820; McCoull, William, et al., ACS Chemical Biology 13.11 (2018): 3131-3141. Bellenie, Benjamin R., et al., Journal of Medicinal Chemistry 63.8 (2020): 4047-4068; Lloyd, Matthew G., et al., Journal of Medicinal Chemistry 64.23 (2021): 17079-17097.

Binding affinity of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein to BCL6 can be determined by, for example, a binding IC₅₀ or Kᵢ value (e.g., using a competition assay), or by a K_{D} value (e.g., using a biophysical assay). A compound with a lower binding IC₅₀ value, as determined under substantially similar conditions, is a more potent binder relative to a compound with a higher binding IC₅₀ value. A compound with a lower binding Kᵢ value, as determined under substantially similar conditions, is a more potent binder relative to a compound with a higher binding Kᵢ value. Similarly, a compound with a lower K_{D} value, as determined under substantially similar conditions, is a more potent binder relative to a compound with a higher K_{D} value. For example, a K_{D} value can be determined by surface plasmon resonance (SPR) or biolayer interferometry; see, e.g., Guo, Weikai, et al., Journal of Medicinal Chemistry 63.2 (2020): 676-695; Lloyd, Matthew G., et al., Journal of Medicinal Chemistry 64.23 (2021): 17079-17097 and International Publication Nos. WO 2019/153080; WO 2019/119144; and WO 2019/119145.

The ability of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (T-b) (e.g., Formula (I-b-1) or (I-b-2))) or Formula (II), or a pharmaceutically acceptable salt thereof, as provided herein to inhibit BCL6 can be determined using an IC₅₀ value. A compound with a lower IC₅₀ value, as determined under substantially similar conditions, is a more potent inhibitor relative to a compound with a higher IC₅₀ value. For example, an IC₅₀ value can be calculated using a FRET (e.g., Homogeneous Time Resolved Fluorescence (HTRF)) assay, where a tagged (e.g., His-tagged) BCL6 protein and tagged (e.g., fluorophore-tagged (e.g., Alexa-Fluor633)) corepressor peptide (e.g., BCOR) are incubated in the presence of compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), and subsequently, the FRET ratio (relative to appropriate controls) can be measured using an appropriate FRET pair (e.g., with an antibody that recognizes the tagged BCL6 protein (e.g., anti-His-Terbium cryptate)). See, e.g., International Publication Nos. WO 2018/108704; WO 2018/215798; WO 2019/197842; WO 2020/104820; WO 2021/074620. As another example, an IC₅₀ value can be calculated using an enzyme-linked immunosorbent assay (ELISA) using a tagged (e.g., biotinylated) corepressor peptide (e.g., BCOR) immobilized on a substrate and a tagged (e.g., FLAG-tagged) BCL6 (e.g., a domain, such as the BTB domain, thereof), where compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II) can be used to prevent the interaction between the corepressor peptide and BCL6, and the interaction between the corepressor peptide and BCL6 can be measured using an antibody to the BCL6 construct (e.g., anti-FLAG antibody). See, e.g., Kamada, Yusuke, et al., Journal of Medicinal Chemistry 60.10 (2017): 4358-4368. As yet another example, an IC₅₀ value can be calculated using a florescence polarization assay with a fluorescently-tagged corepressor peptide (e.g., SMRT) where compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II) can be used to prevent the interaction between the corepressor peptide and BCL6. See, e.g., International Publication No. WO 2019/119144. As another example, a cellular IC₅₀ value can be calculated using a BRET (Bioluminescence Resonance Energy Transfer) assay, where vectors encoding BCL6 and a corepressor peptide (e.g., SMRT), complementarily fused with NanoLuc or HaloTag, can be inserted into cells. The cells can be treated with compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, to determine the effect of the compounds on inhibiting the BCL6-corepressor interaction. See, e.g., International Publication Nos. WO 2018/215798 and WO 2019/197842. In another example, an IC₅₀ value for the inhibition of BCL6 repressor function can be calculated using a luciferase assay, where cells are engineered to express luciferase under the control of one or more BCL6 repressor sites, and the cells can be incubated with compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, to determine the effect of the compounds on the function of the BCL6 repressor. See, e.g., International Publication Nos. WO 2019/119144; WO 2019/119145; WO 2019/153080.

Another exemplary way of evaluating the effect of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is to measure the induction (e.g., fold induction) of genes that are typically repressed by BCL6 (e.g., p53, ATR, CXCR3, CD69, and CDKN1A) using a method such as RT-PCR. See, e.g., Guo, Weikai, et al., Journal of Medicinal Chemistry 63.2 (2020): 676-695.

An exemplary assay for determining the potency of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, includes measuring the effect of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, on cell proliferation and/or viability. Cell proliferation assays can be performed in a number of formats, including 2D and 3D.

Similarly, a cell proliferation assay can be performed with any appropriate cell line, including, for example, A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2. In some embodiments, a cell line that is not dependent on BCL6 and/or that does not have significant expression of BCL6 can be used as a control (e.g., Toledo, H929, MM.1S, or OPM2). As an illustrative example, a 3D cell proliferation assay can include growing cells in a 3D medium, contacting the cells with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, measuring the cellular proliferation using an appropriate reagent (e.g., CELLTITERGLO® 3D), and then comparing the signal from an experiment with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to the signal from a control experiment (e.g., lacking the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (T-b-1) or (I-b-2))) or Formula (II), or a pharmaceutically acceptable salt thereof). As another illustrative example, a 2D cell proliferation assay can include plating cells onto a growth surface, optionally letting the cells grow for a period of time, contacting the cells with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, measuring the cellular proliferation using an appropriate reagent (e.g., CELLTITERGLO®), and then comparing the signal from an experiment with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to the signal from a control experiment (e.g., lacking a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof). Additional cell viability assays include MTT assays, which are colorimetric assays based on the reduction of the tetrazolium dye MTT (3-(4,5-dimethylthiazol-2-yl)-2,5-diphenyltetrazolium bromide) to the insoluble purple formazan, and other similar assays based on related tetrazolium salts, ATPlite assays, and other methods are known in the art. See, for instance, Example B2. See also, e.g., Guo, Weikai, et al., Journal of Medicinal Chemistry 63.2 (2020): 676-695; McCoull, William, et al. ACS Chemical Biology 13.11 (2018): 3131-3141; Lloyd, Matthew G., et al. Journal of Medicinal Chemistry 64.23 (2021): 17079-17097; Bellenie, Benjamin R., et al. Journal of Medicinal Chemistry 63.8 (2020): 4047-4068; and International Publication Nos. WO 2018/215798; WO 2018/215801; WO 2018/219281; WO 2019/119145; WO 2019/153080; and WO 2020/104820.

A cell viability assay can be used to measure the effect of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, on cell death. For example, cells expressing BCL6 protein (e.g., A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2 cells) can be incubated with various concentrations of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, then exposed to a detection reagent (e.g., using a CELLTITER-GLO® Cell Viability Assay kit) to determine cell viability. In some embodiments, the effect on cell viability can be compared to a cell line that is not dependent on BCL6 and/or that does not have significant expression of BCL6 can be used as a control (e.g., Toledo, H929, MM.1S, or OPM2).

A cell viability assay can be used to measure the effect of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, on cell death in combination with an additional therapeutic agent. For example, cells expressing BCL6 protein (e.g., A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2 cells) can be incubated (e.g., for 72 hours or for 120 hours) in a 7×7 dose matrix at various concentrations of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and an additional therapeutic agent (e.g., any of the additional therapeutic agents described herein) (e.g., half-log diluted from 316 to 1 nM), then exposed to a detection reagent (e.g., using a CELLTITER-GLO® Cell Viability Assay kit) to determine cell viability. The combination activity can be assessed by the Bliss independence model: negative values as indication of antagonism, positive as synergy, and a value of zero as additive activity. Bliss scores in the dose matrix can be added up to give a “Bliss sum” value to reflect the overall synergy activity of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent in each cell line. In some embodiments, a cell line that is not dependent on BCL6 and/or that does not have significant expression of BCL6 can be used as a control (e.g., Toledo, H929, MM.1S, or OPM2).

As another example, the potency and/or efficacy of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be evaluated in an animal model, for example, a cell line-derived (CDX) xenograft model (e.g., using an established cancer cell line such as DB, DoHH2, OCI-Ly1, OCI-Ly7, RL, Pfeiffer, SU-DHL-5, SU-DHL-6, WSU-DLCL2, REH, BALL-1, RS4;11, SEMK2, KOPN8, NALM-6, KASUMI-2, RCH-ACV, SUP—B15, BV-173, TOM-1, NALM-20, NALM-21, MUTZ-5, or MHH-CALL-4 (e.g., OCI-Ly1, OCI-Ly7, SU-DHL-5, SU-DHL-6, WSU-DLCL2, DB, RL, Pfeiffer, or DoHH2)), a genetically engineered mouse model (GEMM) or a patient-derived xenograft (PDX) model. For example, a PDX model can be run in immunodeficient mice (e.g., athymic nude, outbred homozygous (e.g., Crl:NU(NCr)-Foxn1^{nu}) or Fox Chase SCID (CB17/Icr-Prkd^{scid}/IcrIcoCrl), mice). The mice can be female, 6-12 weeks old at tumor implantation and have access to food and water ad libitum. Approximately 70 mg of a tumor can be implanted subcutaneously in the right flank of each mouse. Following implantation, tumors can be measured weekly and once the tumor volumes reach 150-300 mm³, the mice can be randomized into treatment and control groups. In some embodiments, one or more experimental arms can be added to evaluate pharmacokinetics and/or pharmacodynamics. The mice can be treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, (e.g., via IP or PO administration) and optionally an additional therapy or therapeutic agent (e.g., any of the additional therapies or therapeutic agents described herein). Throughout the study, health condition, body weight and tumor volumes of the mice can be recorded on a weekly basis. The mice can be sacrificed at 28 days or when the tumor reaches 1 cm³, and the tumors can be evaluated (e.g., by tumor weight, by tumor volume). At the end of each study the Best Response can be calculated for each treatment arm. Best Response is defined as the minimum value of AVolumet for t>10 days. Best Responses between the control arm(s) and the treatment arm(s) can be compared to determine if the treatment(s) work better than the control(s). In some embodiments, tumor samples can also be collected at the end of each study and relevant proteins (e.g., BCL6) can be measured to determine if the treatment has a better protein modulation profile compared to a control. In some embodiments, tumor samples and/or blood samples can also be collected at the end of each study and analyzed for altered gene expression activity (e.g., altered ARID3A, ARID3B, ATR, B2M, BANK1, BATF, BCL11A, BCL2, BCL2A1, BLIMP1, BM11, CASP8, CCND1, CCND2, CCR6, CCR7, CD38, CD44, CD69, CDKN1A, CDKN1B, CFLAR/FLIP, CHEK1, CXCR4, CXCR5, DR5, EBI2, ETV6, FCMR, FGD4, ID2, IFITM1, IFITM2, IFNAR2, IFNGR1, IL10, IL10RB, IL7R, CXCL10, IRF1, IRF4, IRF7, IRF9, JAK3, JARID2, JUN, KLF2, LITAF, MCL1, MIP-1a, MYC, MYD88, NFKBIE, NOTCH2, PDL1, PIM1, PPP3R1, PRDM1, PTEN, S1PR1, SHP1, STAT1, STAT3, STAT5A, TLR1, TLR4, TLR7, TLR9, TNF-R2, TOX, TP53, ZEB2, and/or ZNF608 expression levels). For pharmacokinetic and pharmacodynamic studies, tumor and/or blood samples from the mice can be obtained at the same or different time points than efficacy studies. For example, for pharmacokinetic and pharmacodynamic studies, tumor and/or blood samples from the mice can be obtained at Day 1, 3 and/or 5, with collections at 6, 12 and/or 24 hours post-dosing, and relevant proteins can be measured in the tumor samples and pharmacokinetic studies can be performed on the blood samples or a portion thereof (e.g., plasma). In some embodiments, the PDX is a model of B-ALL (e.g., Philadelphia chromosome positive B-ALL, Philadelphia chromosome negative B-ALL, or B-ALL with an MLL-Af4 fusion, an MLL-Af6 fusion, an MLL-Af9 fusion, an MLL-ENL fusion, or an MLL-PTD fusion), DLBCL, FL, MCL, Burkitt lymphoma, or peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)). See, e.g., Guo, Weikai, et al., Journal of Medicinal Chemistry 63.2 (2020): 676-695.

In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, exhibit activity in a model of an autoimmune disease. Exemplary assays can be found in, for example, International Publication Nos. WO 2020/014599 and WO 2021/074620.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof, can be assessed for its ability to modulate (e.g., decrease) IgG antibody production and/or modulate (e.g., decrease) germinal center formation in an animal (e.g., mouse) following challenge with a T cell-dependent antigen (e.g., keyhole limpet haemocyanin (KLH)). For example, KLH can be administered to mice (e.g., C57BL/6 mice), followed by administration of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, for a period of time (e.g., 14 days). Following sacrifice, serum samples from the mice can be analyzed for IgG specific for KLH, for example, by ELISA. Similarly, germinal centers can be detected using immunohistological staining using, e.g., peanut agglutinin (PNA). See, e.g., Example 1 of WO 2020/014599.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be assessed for its ability to modulate (e.g., decrease) the number of germinal center B cells in an animal (e.g., mouse) following immunization with an antigen. For example, animals (e.g., mice) can be immunized with Complete Freund's Adjuvant (CFA), and after a period of time (e.g., 8 days), the animals can be sacrificed and the spleens harvested. The spleens can be processed into suspension and cultured in the presence of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof. The cells can then be analyzed, for example, for the number of germinal center B cells (e.g., by flow cytometry using the lineage markers GL7 and CD95). See, e.g., WO 2021/074620.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof, can be assessed for its ability to improve one or more symptoms, biomarkers, or other signs of an autoimmune disease in an animal model of the autoimmune disease.

Experimental autoimmune encephalitis (EAE) can be used as an animal model of inflammatory diseases of the CNS, including multiple sclerosis (MS) and neuromyelitis optica. In some cases, EAE can be induced in animals (e.g., mice), for example, via immunization with recombinant human myelin oligodendrocyte glycoprotein (MOG) or a fragment thereof (e.g., MOG₁₋₁₂₅), myelin basic protein, and/or proteolipid protein. Following induction of EAE, the animals can be treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated by, for example, EAE severity, B cell depletion, or both. See, e.g., Constantinescu, Cris S., et al. British Journal of Pharmacology 164.4 (2011): 1079-1106; Monson, Nancy L., et al. PloS One 6.2 (2011): e17103.

In some embodiments, the autoimmune disease is anti-synthetase syndrome. For example, a model of anti-synthetase syndrome, can be induced in susceptible mice (e.g., C57BL/6, B6.G7, and/or NOD.Idd3/5) by immunization with histidyl-tRNA synthetase (e.g., murine histidyl-tRNA synthetase or human histidyl-tRNA synthetase), or a fragment thereof. Treatment with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, may begin at immunization and continue for a period of time (e.g., short term (e.g., 10-14 days) or long term (e.g., 8-16 weeks)), then the mice can be sacrificed. The mice can be evaluated, for example, for generation of histidyl-tRNA synthetase-specific antibody (e.g., via immunoprecipitation, ELISA, and/or flow cytometry), tissue (e.g., lung and/or muscle) inflammation (e.g., by pathologist review), or a combination thereof. See e.g., Katsumata, Yasuhiro, et al. Journal of Autoimmunity 29.2-3 (2007): 174-186; Katsumata, Yasuhiro, et al. Journal of Autoimmunity 29.2-3 (2007): 174-186; Ascherman, Dana P. Current Rheumatology Reports 17 (2015): 1-7; and Konishi, Risa, Yuki Ichimura, and Naoko Okiyama. Immunological Medicine 46.1 (2023): 9-14.

In some embodiments, the autoimmune disease is arthritis (e.g., rheumatoid arthritis or inflammatory arthritis). For example, a mouse model of rheumatoid arthritis, collagen induced arthritis (CIA), can be induced in susceptible mice (e.g., DBA/1 or HLA-DR) by immunization with type II collagen (CII). Treatment with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, may begin at immunization and continue for a period of time (e.g., 6 weeks). The mice can be evaluated, for example, for clinical scores (e.g., inflammation of an arthritic limb, foot thickness, paw volume (e.g., using a plethysmometer), or a combination thereof), generation of CII-specific antibody, B cell depletion, or a combination thereof. See e.g., Example 2 of WO 2020/014599; Brand, David D., et al. Nature Protocols 2.5 (2007): 1269-1275. Additional animal models of arthritis (e.g., inflammatory arthritis) are known in the art, such as K/BxN mice. In some embodiments, such mice can be treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and evaluated, for example, in similar ways to CII-immunized mice, or additionally, titering for autoantibodies, such as those against glucose-6-phosphate isomerase. See, e.g., Huang, Haochu, Christophe Benoist, and Diane Mathis. Proceedings of the National Academy of Sciences 107.10 (2010): 4658-4663; and Pigott, Elizabeth, and Laura Mandik-Nayak. Arthritis & Rheumatism 64.7 (2012): 2169-2178.

In some embodiments, the autoimmune disease is graft-versus-host disease (e.g., chronic graft-versus-host disease). In some cases, an animal model of graft-versus-host disease can be in animals (e.g., mice) conditioned with high-dose cyclophosphamide and lethal total-body irradiation (TBI) rescued with bone marrow optionally including allogeneic splenocytes or purified T cells. Administration of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, for a period of time can follow. The animals can be evaluated by, for example, pulmonary function tests, by immunohistochemistry to evaluate the presence of autoantibodies, or by examining sections of the spleen for germinal centers, including for example, examining for B cell depletion (e.g., germinal center B cell depletion). See, e.g., Srinivasan, Mathangi, et al. Blood, The Journal of the American Society of Hematology 119.6 (2012): 1570-1580; and Paz, Katelyn, et al. Blood, The Journal of the American Society of Hematology 133.1 (2019): 94-99. See also, e.g., Dubovsky, Jason A., et al. The Journal of Clinical Investigation 124.11 (2014): 4867-4876.

In some embodiments, the autoimmune disease is IgG4-related disease (IgG4-RD). In some cases, an animal model of IgG4-RD can be generated by injecting mice with IgGs (e.g., IgG1 and/or IgG4) derived from human IgG4-RD patients. Such mice can be treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated by measuring, for example, pancreatic and/or salivary tissue damage, B cell depletion, or both. See, e.g., Shiokawa, Masahiro, et al. Gut 65.8 (2016): 1322-1332.

In some embodiments, the autoimmune disease is lupus (e.g., lupus erythematosus). In some cases, an animal model of lupus is MRL/lpr mice, which can display high expression of Tfh-associated molecules such as ICOS, PD-1, BCL-6, and IL-21 and produce autoantibodies to nuclear components, develop nephritis, arthritis, and skin lesions. Such mice can be treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated by, for example, measuring B cell depletion (e.g., germinal center B cell depletion), identifying the presence and/or severity of glomerulonephritis, autoantibody titers (e.g., anti-RNA antibody titers, anti-nuclear antibody titers, and/or anti-dsDNA antibody titers), IL-21 expression, and/or amount of activated CD4+ T cells. See, e.g., Ahuja, Anupama, et al. The Journal of Immunology 179.5 (2007): 3351-3361; Shen, Chunxiu, et al. Journal of Cellular and Molecular Medicine 25.17 (2021): 8329-8337; and Marinov, Anthony D., et al. Arthritis & Rheumatology 73.5 (2021): 826-836. Additional animal models of lupus are known in the art, such as NZB/W mice, which can be treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and evaluated in similar ways to the MRL/lpr mice. See, e.g., Wang, Wensheng, et al. The Journal of Immunology 192.7 (2014): 3011-3020; and Kansal, Rita, et al. Science Translational Medicine 11.482 (2019): eaav1648.

In some embodiments, the autoimmune disease is myasthenia gravis (e.g., muscle-specific tyrosine kinase (MuSK) positive myasthenia gravis). For example, an animal model (e.g., a rat model, a mouse model, or a rabbit model) of myasthenia gravis can be generated by immunizing the animal with acetylcholine receptor from Torpedo (e.g., Torpedo californica) or Electrophorus (e.g., Electrophorus electricus) electric organs, or recombinant acetylcholine receptor protein or fragments thereof. Treatment with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can begin, for example, approximately 4 weeks after immunization, but can sometimes begin earlier or later. The rodents can be evaluated, for example, for clinical scores (e.g., grasping and/or lifting of a weight, while looking for tremor, hunched posture, muscle strength, and signs of fatigue), body weight, compound muscle action potential (e.g., using electromyography), anti-acetylcholine receptor antibodies, B cell depletion, or a combination thereof. See, e.g., Mori, Shuuichi, et al. The American Journal of Pathology 180.2 (2012): 798-810; Xin, Ning, et al. Molecular and Cellular Neuroscience 58 (2014): 85-94; and Losen, Mario, et al. Experimental Neurology 270 (2015): 18-28.

In some embodiments, the autoimmune disease is multiple sclerosis (MS). In some embodiments, the MS is clinically isolated syndrome (CIS), relapsing-remitting MS (RRMS), primary progressive MS (PPMS), or secondary progressive MS (SPMS). In some embodiments, animal models of MS can be based on EAE, for example, relapsing-remitting EAE in SJL/J mice (e.g., via immunization with proteolipid protein(PLP)₁₃₉₋₁₅₁), chronic EAE in C57BL/6J mice (e.g., via immunization with MOG₃₅₋₅₅), or EAE in transgenic mice (e.g., via a T cell clone expressing Vα and Vβ chains reacting specifically to MOG₃₅₋₅₅, or a B cell heavy chain knock-in mouse). In some embodiments, the animal (e.g., mouse) model is generated via infection with a picornavirus, such as Theiler's murine encephalitis virus. In some embodiments, the animal (e.g., mouse) model is generated by feeding C57BL/6 mice with cuprizone (e.g., 0.2% for 6 weeks). In some embodiments, the animal (e.g., mouse) model is generated via lysolecithin injection (e.g., in SJL/J mice). Treatment with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be followed, for example, by analysis of the animals for levels of myelin-specific T cells, B cell depletion, reduction of inflammatory lesions, axonal degeneration, protection or reversal of cuprizone or lysolecithin-induced demyelination, or a combination thereof. See, e.g., Procaccini, Claudio, et al. European Journal of Pharmacology 759 (2015): 182-191.

In some embodiments, the autoimmune disease is neuromyelitis optica (NMO). In some cases, animal models of NMO include administration of antibodies against astrocyte water channel aquaporin-4 (AQP4), called AQP4-IgG or NMO-IgG, to various CNS tissues, such as the brain. In some embodiments, the administration of the antibodies is to an animal with EAE. The AQP4-IgG may be recombinant or derived from human NMO patients. Treatment with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof, can be followed, for example, by analysis of the sacrificed animal for slowing or reversal of astrocyte demyelination, number of macrophages, number of activated microglia, number of activated neutrophils, B cell depletion, or a combination thereof. See, e.g., Bennett, Jeffrey L., et al. Annals of Neurology: Official Journal of the American Neurological Association and the Child Neurology Society 66.5 (2009): 617-629; Saini, Harleen, et al. BMC Neurology 13.1 (2013): 1-9; Oji, Satoru, et al. PloS One 11.3 (2016): e0151244; Peschl, Patrick, et al. Journal of Neuroinflammation 14.1 (2017): 1-14; and Duan, Tianjiao, and Alan S. Verkman. Brain Pathology 30.1 (2020): 13-25.

In some embodiments, the autoimmune disease is pemphigus (e.g., pemphigus vulgaris). In some cases, an animal (e.g., mouse) model of pemphigus can be generated using adoptive transfer of peripheral lymphocytes from Dsg3 knockout animals to immune-deficient but desmoglein 3-expressing recipient mice to create an artificial immune state in the recipient animals. In some cases, an animal (e.g., mouse) model of pemphigus can be generated using an animal with a MHC class II-null background that expresses the pemphigus-associated HLA-DRB1*0402 allele, which after immunization with recombinant human DSG3, produce anti-human DSG3 antibodies. Treatment of these models with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be followed, for example, by analysis of the B cell repertoires of the animals for anti-desmoglein B-cell clones, B cell depletion, or both. See, e.g., Kasperkiewicz, Michael, et al. Nature Reviews Disease Primers 3.1 (2017): 1-18.

In some embodiments, the autoimmune disease is Sjogren's syndrome. In some cases, an animal model of Sjogren's syndrome is a NOD mouse, which sometimes has further genetic manipulation or crosses (e.g., NOD.B10.H2^{b} or C57BL/6.NOTDc3.NODclt) to recapitulate the symptoms of the syndrome. Such mice can be treated with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated, for example, for salivary and lacrimal gland secretory flow rates, salivary protein content, autoantibodies to exocrine gland proteins (e.g., anti-Sjogren's syndrome A (SSA) antibodies, anti-Sjogren's syndrome B (SSB) antibodies, and/or anti-muscarinic acetylcholine 3 receptor (M3R) antibodies), B cell depletion, or a combination thereof. Robinson, Christopher P., et al. Arthritis & Rheumatism 41.1 (1998): 150-156; Cha, Seunghee, et al. “Arthritis & Rheumatism 46.5 (2002): 1390-1398; and Ohno, Seiji, et al. Autoimmunity 45.7 (2012): 540-546. Additional animal models of Sjogren's syndrome are known in the art and include, for example, NFS/sld mice, IQI/Jic mice, Aly/aly mice, and mice immunized with M3R peptides, which can be evaluated, in some cases, for the same parameters as NOD mice. See, e.g., Iizuka, Mana, et al. Journal of Autoimmunity 35.4 (2010): 383-389; and Park, Young-Seok, Adrienne E Gauna, and Seunghee Cha. Current Pharmaceutical Design 21.18 (2015): 2350-2364.

The pharmacokinetic parameters of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be evaluated in an animal model, for instance, a mouse model, a rat model, a dog model, or a nonhuman primate (e.g., cynomolgus monkey) model. Pharmacokinetics (PK) studies can be conducted in an animal model (e.g., male CD-1 mice) by two exemplary delivery routes: intravenous (IV) injection and oral (PO), for example, oral gavage. Animals in the IV group (e.g., n=3) are allowed free access to food and water; animals in the PO group are allowed free access to food or are fasted for 6-8 hours prior to dosing. A compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be formulated in solution for the IV route and solution or suspension for the PO route. On the day of the experiment, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be administered via vein injection (e.g., at 1 mg/kg) for IV route or via oral gavage (e.g., at 3 to 90 mg/kg, or 3 to 10 mg/kg, such as 10 mg/kg) for PO route. In some cases, the animals can be orally pre-dosed with a cytochrome P450 inhibitor (e.g., 1-aminobenzotriazole) prior to (e.g., 16 hours prior to) dosing the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. Blood samples can be collected via serial bleeding (e.g., at 8 timepoints from 0.83 to 24 hours post dose). At each timepoint, blood (e.g., about 30 μL to about 125 μL, or about 75 μL to about 125 μL) can be collected (e.g., via the saphenous vein) in a tube containing an anti-coagulant (e.g., K₂EDTA). Blood samples can be put on wet ice and centrifuged (e.g., at 2000×g for 4-10 minutes) to obtain plasma samples. Plasma samples can be diluted (e.g., with an equal volume of pH 3.0 phosphate buffer or with an equal volume of pH 5.0 sodium citrate) and submitted to LC-MS/MS for sample analysis. Pharmacokinetics parameters, including clearance (CL), volume of distribution (V_{d}), maximum plasma concentration (Cₘₐₓ), time of maximum plasma concentration (tₘₐₓ), half-life (t_{1/2}), area under the curve (AUC), and oral bioavailability (% F) can be calculated using a non-compartmental model.

In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 4%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 10%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 20%.

In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 30%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 40%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 60%.

In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 80%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 4% to about 90% (e.g., about 4% to about 80%, about 4% to about 60%, about 4% to about 40%, about 4% to about 20%, about 4% to about 10%, about 20% to about 40%, about 40% to about 60%, about 60% to about 80%, or about 70% to about 90%). In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 4% to about 20%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 20% to about 40%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 40% to about 60%. In some embodiments, the % F for a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 50% to about 80%.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not a substrate of a human cytochrome P450 enzyme. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not a substrate of a human cytochrome P450 enzyme where >25% of clearance is attributed to that enzyme. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (T-bb) (e.g., Formula (T-bb-1) or (I-bb-2)), or Formula (T-b) (e.g., Formula (I-b-1) or (I-b-2))) or Formula (II), or a pharmaceutically acceptable salt thereof, is not an inhibitor and/or an inducer of one or more human cytochrome P450 enzymes. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (T-a-4), (I-a-5), or (I-a-6)), Formula (T-bb) (e.g., Formula (I-bb-1) or (I-bb-2)), or Formula (I-b) (e.g., Formula (I-b-1) or (I-b-2))) or Formula (TT), or a pharmaceutically acceptable salt thereof, is not an inhibitor and/or an inducer of one or more human cytochrome P450 enzymes, where the IC₅₀ and/or EC₅₀ for the one or more human cytochrome P450 enzymes, respectively, is at a concentration to be significantly greater than the estimated free fraction concentration of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (T-bb) (e.g., Formula (I-bb-1) or (I-bb-2)), or Formula (I-b) (e.g., Formula (T-b-1) or (I-b-2))) or Formula (II), or a pharmaceutically acceptable salt thereof, at a clinically relevant dose.

Exemplary human cytochrome P450 enzymes include those in the CYP1, CYP2, and CYP3 families. For example, any one of CYP1A1, CYP1A2, CYP1B1, CYP2A6, CYP2B6, CYP2C8, CYP2C9, CYP2C18, CYP2C19, CYP2D6, CYP2J2, CYP2S1, CYP2E1, CYP3A4, and CYP3A5.

In some embodiments, no single cytochrome P450 enzyme is responsible for greater than or equal to 25% of the elimination of the compound of Formula (I) (e.g., Formula (I-aa) (e.g., Formula (T-aa-1), (I-aa-2), (I-aa-3), (I-aa-4), (I-aa-5), or (I-aa-6)), Formula (I-a) (e.g., Formula (I-a-1), (T-a-2), (I-a-3), (I-a-4), (I-a-5), or (I-a-6)), Formula (T-bb) (e.g., Formula (I-bb-1) or (I-bb-2)), or Formula (I-b) (e.g., Formula (I-b-1) or (I-b-2))) or Formula (TT), or a pharmaceutically acceptable salt thereof. Cytochrome P450 inhibition and/or inducing activity can be determined using any appropriate assay, such as those described in the guidance document “In Vitro Drug Interaction Studies Cytochrome P450 Enzyme- and Transporter-Mediated Drug Interactions” provided by the U.S. F.D.A. in January 2020. For example, evaluation of cytochrome P450 inhibition can be performed in in vitro studies, in both a reversible and time-dependent manner. In an in vitro inhibition study, the ratio of intrinsic clearance values of a probe substrate for an enzymatic pathway in the absence and in the presence of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be calculated; this ratio is referred to as R₁ for reversible inhibition, where R₁₌₁+(I_{max,u}/K_{i,u}), and I_{max,u} is the maximal unbound plasma concentration of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and K_{i,u} is the unbound inhibition constant determined in vivo. Specifically, for CYP3A, R^{1,}_{gut} can be calculated where R_{1,gut}=1+(I_{gut}+K_{i,u}),and where I_{gut} is the intestinal luminal concentration of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, calculated as the dose/250 mL. The time-dependent inhibition ratio R₂ can similarly be calculated, where R₂=(k_{obs}+k_{deg})/k_{deg}, and k_{obs} is the observed (apparent first order) inactivation rate of the affected cytochrome P450 calculated by k_{obs}=(k_{inact}*50*I_{max,u})/(K_{1,u}+50*I_{max,u}), k_{deg} is the apparent first-order degradation rate constant of the affected cytochrome P450, K_{1,u} is the unbound concentration of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, causing half maximal inactivation, and k_{inact} is the maximal inactivation rate constant. If R₁≥1.02, R₂≥1.25, and/or R_{1,gut}≥11, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (T-bb) (e.g., Formula (T-bb-1) or (I-bb-2)), or Formula (T-b) (e.g., Formula (I-b-1) or (I-b-2))) or Formula (II), or a pharmaceutically acceptable salt thereof, may be an inhibitor of a cytochrome P450, and the drug-drug interaction (DDI) potential can be further investigated using mechanistic models and/or conducting a clinical DDI study with a sensitive index substrate. For example, evaluation of cytochrome P450 induction can be performed via the fold-change method, wherein the fold-change in cytochrome P450 enzyme mRNA levels when incubated with the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, by using a cutoff determined from known positive and negative controls to calibrate the system. For example, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (T-b) (e.g., Formula (I-b-1) or (I-b-2))) or Formula (II), or a pharmaceutically acceptable salt thereof, is interpreted as an inducer if: (1) it increased mRNA expression of a cytochrome P450 enzyme in a concentration-dependent manner; and (2) the fold change of cytochrome P450 mRNA expression relative to the vehicle control is ≥2-fold at the expected hepatic concentrations of the drug. As another example, evaluation of cytochrome P450 induction can be performed by a correlation method, wherein correlation methods are used to predict the magnitude of a clinical induction effect (e.g., AUC ratio of an index substrate in the presence and absence of inducers) of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (T-a-2), (T-a-3), (T-a-4), (T-a-5), or (I-a-6)), Formula (T-bb) (e.g., Formula (T-bb-1) or (I-bb-2)), or Formula (I-b) (e.g., Formula (T-b-1) or (I-b-2))) or Formula (TT), or a pharmaceutically acceptable salt thereof, according to a calibration curve of relative induction scores (RIS) or I_{max,u}/EC₅₀ for a set of known inducers of the same cytochrome P450. If the predicted magnitude is more than a predefined cut-off (e.g., AUC ratio<0.8), a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (T-bb) (e.g., Formula (T-bb-1) or (I-bb-2)), or Formula (I-b) (e.g., Formula (I-b-1) or (I-b-2))) or Formula (II), or a pharmaceutically acceptable salt thereof, is considered to have induction potential in vivo.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not a hERG inhibitor. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 60 nM (e.g., greater than 100 nM, 300 nM, 500 nM, 1 μM, 3 μM, 5 μM, 10 μM, 20 μM, or 30 μM). For example, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 500 nM (e.g., 1 μM, 3 μM, 5 μM, 10 μM, 20 μM, or 30 μM).

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 1 μM (e.g., greater than 3 μM, 5 μM, 10 μM, 20 μM, or 30 μM). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 10 μM (e.g., greater than 20 μM or 30 μM). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 30 PM.

Heterobifunctional degraders can, in some cases, induce the degradation of off-target proteins. For heterobifunctional degraders that utilize CRBN, common off-target proteins that can be degraded include GSPT1, IKZF1, IKZF2, IKZF3, and/or CK1α. This degradation is generally believed to be due to the E3 binding moiety of the heterobifunctional degrader facilitating ternary complex formation between the off-target protein and CRBN. GSPT1 is a translation termination factor, and CK1a is a kinase that is involved in many key cellular processes including cell cycle progression and chromosome segregation; these are both commonly essential genes, so undesired degradation of either or both may lead to nonspecific cytotoxicity. The IKZF proteins are zinc finger transcription factors that are involved with cell fate during hematopoiesis, and degradation of these proteins has been associated with hematotoxicity. See, e.g., Moreau, Kevin, et al. British Journal of Pharmacology 177.8 (2020): 1709-1718.

In some embodiments, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, can exhibit potent and selective induction of degradation of a BCL6 protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutically acceptable salt thereof, can selectively target a BCL6 protein for degradation over a second protein (e.g., GSPT1, IKZF1, IKZF2, IKZF3, CK1α, C6orfl32, CAMP (cathelicidin antimicrobial peptide), CCNA2 (cyclin-A2), FSP1 (ferroptosis suppressor protein 1, also known as AIFM2), JCHAIN (immunoglobin J chain), NLRP7 (NACHT, LRR, PYD domains-containing protein 7), PTTG1 (securin), and/or TPX2 (targeting protein for Xklp2)). CAMP is an antimicrobial protein that is an integral part of the innate immune system, and it binds to bacterial lipopolysaccharides. CCNA2 controls both the GUS and the G2/M transition phases of the cell cycle. FSP1 is an oxidoreductase that is an inhibitor of ferroptosis. JCHAIN links two monomer units of either IgM or IgA; the J chain-joined dimer is a nucleating unit of the IgM pentamer, and the J chain-joined dimer of IgA induces dimers or larger polymers. NLRP7 Inhibits CASP1/caspase-1-dependent IL1B secretion. PTTG1 is key for chromosomal stability and negatively regulates TP53. TPX2 is required for the normal assembly of mitotic spindles.

As used herein, “selective” or “selectively”, when referring to a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in a protein degradation assay, indicates at least a 5-fold (e.g., at least a 10-fold, at least a 25-fold, at least a 50-fold, or at least a 100-fold) superior performance in the protein degradation assay for a specified protein with reference to a comparator protein in the assay.

In some embodiments, the compounds provided herein can exhibit potency (e.g., nanomolar potency) against a BCL6 protein with minimal activity (e.g., single digit micromolar potency, for example, potency greater than 1 μM (e.g., greater than 3 μM, 5 μM, 10 μM, 20 PM, or 30 μM)) against a second protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading (e.g., as measured by Yin, DC₅₀, and/or Dₘₐₓ values) a second protein (e.g., GSPT1, IKZF1, IKZF2, IKZF3, CK1α, C6orfl32, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit greater induction of degradation of a BCL6 protein relative to induction of degradation (e.g., as measured by Yₘᵢₙ, DC₅₀, and/or Dₘₐₓ values) of a second protein (e.g., GSPT1, IKZF1, IKZF2, IKZF3, CK1α, C6orfl32, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit up to 1000-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 2-fold to about 10-fold greater induction of degradation of a BCL6 protein relative to induction of degradation of a second protein (e.g., GSPT1, IKZF1, IKZF2, IKZF3, CK1α, C6orfl32, CAMP, CCNA2, FSP1, JCHAIN, NLRP7, PTTG1, and/or TPX2) (e.g., as measured by Yₘᵢₙ, DC₅₀, and/or Dₘₐₓ values). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 10-fold to about 100-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 100-fold to about 1000-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 1000-fold to about 10000-fold greater induction of degradation of a BCL6 protein relative to induction of degradation of a second protein. In some embodiments, the second protein is selected from the group consisting of GSPT1, IKZF1, IKZF2, IKZF3, CK1α, C6orfl32, CAMP, CCNA2, FSP1, JCHAIN, NLRP7, PTTG1, and TPX2. In some embodiments, the second protein is selected from the group consisting of GSPT1, IKZF1, IKZF2, IKZF3, and CK1α. In some embodiments, the second protein is selected from the group consisting of C6orfl32, CAMP, CCNA2, FSP1, JCHAIN, NLRP7, PTTG1, and TPX2. In some embodiments, the second protein is C6orfl32. In some embodiments, the second protein is CAMP. In some embodiments, the second protein is CCNA2. In some embodiments, the second protein is FSP1.

In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with similar activity against a second protein (i.e., less than 2-fold greater activity against a BCL6 protein than against a second protein and no more than 2-fold greater activity against the second protein than against the BCL6 protein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit similar induction of degradation of a BCL6 protein relative to induction of degradation (e.g., as measured by Yₘᵢₙ, DC₅₀, and/or Dₘₐₓ values) of a second protein (i.e., less than 2-fold difference greater induction of degradation of a BCL6 protein than induction of degradation of a second protein and no more than 2-fold greater activity against the second protein than against a BCL6 protein) (e.g., GSPT1, IKZF1, IKZF2, IKZF3, CK1α, C6orfl32, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (T-b) (e.g., Formula (I-b-1) or (I-b-2))) or Formula (II), or a pharmaceutically acceptable salt thereof, can exhibit less than 2-fold greater induction of degradation of a BCL6 protein relative to induction of degradation of a second protein. In some embodiments, the second protein is selected from the group consisting of GSPT1, IKZF1, IKZF2, IKZF3, CK1α, C6orfl32, CAMP, CCNA2, FSP1, JCHAIN, NLRP7, PTTG1, and TPX2. In some embodiments, the second protein is selected from the group consisting of GSPT1, IKZF1, IKZF2, IKZF3, and CK1α. In some embodiments, the second protein is selected from the group consisting of C6orfl32, CAMP, CCNA2, FSP1, JCHAIN, NLRP7, PTTG1, and TPX2. In some embodiments, the second protein is C6orfl32. In some embodiments, the second protein is CAMP. In some embodiments, the second protein is CCNA2. In some embodiments, the second protein is FSP1. In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with minimal activity against a second protein (e.g., as measured by a proteomics assay, for example, a<20% reduction in protein abundance as measured in the proteomics assay described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading (e.g., as measured by abundance in a proteomic assay) a second protein (e.g., C6orfl32, CAMP, CCNA2, FSP1, JCHAIN, NLRP7, PTTG, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit greater induction of degradation of a BCL6 protein relative to induction of degradation (e.g., as measured by abundance in a proteomic assay, for example, the proteomics assay as described herein) of a second protein (e.g., C6orfl32, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of a second protein (e.g., C6orfl32, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit up to 1000-fold greater induction of degradation of a BCL6 protein relative to induction of degradation of a second protein (e.g., C6orfl32, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (T-b) (e.g., Formula (I-b-1) or (I-b-2))) or Formula (II), or a pharmaceutically acceptable salt thereof, can exhibit from about 2-fold to about 10-fold greater induction of degradation of a BCL6 protein relative to induction of degradation of a second protein (e.g., C6orfl32, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 10-fold to about 100-fold greater induction of degradation of a BCL6 protein relative to induction of degradation of a second protein (e.g., C6orfl32, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 100-fold to about 1000-fold greater induction of degradation of a BCL6 protein relative to induction of degradation of a second protein (e.g., C6orfl32, CAMP, CCNA2, FSP1, JCHAIN, NLRP7, PTTG1, and/or TPX2) (e.g., as measured by abundance in a proteomic assay, for example, the proteomics assay as described herein). In some embodiments, the second protein is selected from the group consisting of GSPT1, IKZF1, IKZF2, IKZF3, CK1α, C6orfl32, CAMP, CCNA2, FSP1, JCHAIN, NLRP7, PTTG1, and TPX2. In some embodiments, the second protein is selected from the group consisting of GSPT1, IKZF1, IKZF2, IKZF3, and CK1α. In some embodiments, the second protein is selected from the group consisting of C6orfl32, CAMP, CCNA2, FSP1, JCHAIN, NLRP7, PTTG1, and TPX2. In some embodiments, the second protein is C6orfl32. In some embodiments, the second protein is CAMP. In some embodiments, the second protein is CCNA2. In some embodiments, the second protein is FSP1. In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with minimal activity against any other detectable protein (e.g., as measured by a proteomics assay, for example, a<20% reduction in protein abundance as measured in the proteomics assay described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading (e.g., as measured by abundance in a proteomic assay, for example, the proteomics assay as described herein) any other detectable protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit greater induction of degradation of a BCL6 protein relative to induction of degradation (e.g., as measured by abundance in a proteomic assay, for example, the proteomics assay as described herein) of any other detectable protein. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit up to 1000-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 2-fold to about 10-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 10-fold to about 100-fold greater induction of degradation of a BCL6 protein relative to induction of degradation of any other detectable protein (e.g., as measured by abundance in a proteomic assay, for example, the proteomics assay as described herein).

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading one or more additional proteins as measured by abundance in a proteomic assay. An exemplary proteomic experiment follows. OCI-Lyl (DSMZ: ACC 722) cells are incubated with 100 nM of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or dimethyl sulfoxide (DMSO), for six hours. The cells are then washed two times with phosphate buffered saline and collected. Cells are lysed to extract total proteins, and total proteins are prepared for mass spectrometry analysis according to the protocol for the EASYPEP™ MS Sample Prep Kit (Fisher Scientific). In brief, proteins are reduced with dithiothreitol, alkylated with iodoacetamide, and digested with Trypsin and LysC enzyme. The resulting peptides are labeled with TMTPRO™ 18plex reagents (Fisher Scientific) according to the manufacturer protocol.

Labeled peptides from each sample are mixed together in equal volumes, and the peptide mixture is separated by basic reverse-phase chromatography. A total of 85 fractions are combined into 18 pooled fractions. The pooled fractions are dried with a centrivap and resuspended in 5% acetonitrile, 0.1% formic acid for mass spectrometry analysis. Peptide abundance is quantified by tandem mass spectrometry using a Vanquish Neo chromatography system (Fisher Scientific) and Orbitrap FUSION™ LUMOS™ mass spectrometer (Fisher Scientific). Briefly, two micrograms of total peptides are loaded on a two-centimeter C8 trap column followed by a 50-centimeter C18 column. Data-dependent acquisition is performed to obtain peptide sequence and abundance information. Peptide and protein abundances are determined using the PROTEOME DISCOVERER™ software and the Homo sapiens proteome database (TaxID 9606), and the results are filtered to FDR<0.01. Significance thresholds are set to p-value<0.001 and abundance fold-change<50%.

Provided herein is a method of treating a cancer in a subject in need thereof, the method comprising administering to the subject a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the subject is treatment naive with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof as a monotherapy. In some embodiments, the subject is treatment naive with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a monotherapy. In some embodiments, the subject is treatment naive with respect to the cancer. In some embodiments, the subject has received one or more lines of previous therapy for the cancer.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for the treatment of cancer, for example, any of the cancers provided herein.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a medicament for the treatment of cancer, for example, any of the cancers provided herein.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in the manufacture of a medicament for the treatment of cancer, for example, any of the cancers provided herein.

Provided herein is a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament. Also provided herein is a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament for the treatment of cancer, for example, any of the cancers provided herein.

Provided herein is a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use in treating a cancer, for example, any of the cancers provided herein.

As used herein, treatment of a cancer can include treatment of a primary tumor (i.e., non-metastatic cancer) (e.g., as first, second, third, or later line of therapy, including, but not limited to, the relapsed/refractory setting), treatment of a metastatic (or secondary) tumor, neoadjuvant therapy (e.g., before treatment with an additional therapy or therapeutic agent, such as surgery, radiation, chemotherapy, or a line of therapy), adjuvant therapy (e.g., following treatment with an additional therapy or therapeutic agent, such as surgery, radiation, chemotherapy, or a line of therapy), or maintenance therapy (e.g., treatment following response to an additional therapy or therapeutic agent, such as surgery, radiation, chemotherapy, or a line of therapy).

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a primary tumor. In some embodiments, the subject is treatment naive with respect to the cancer. In some embodiments, the subject has received one or more lines of therapy for the cancer. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R—CHOP, G-CHOP, R-EPOCH, CVP, CVAD, R², R-CODOX-M, R-IVAC, DA-EPOCH-R, cell-based therapy, or two or more thereof.

In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a metastatic tumor. In some embodiments, the subject is treatment naive with respect to the metastatic tumor. In some embodiments, the subject has received one or more lines of therapy for the secondary tumor. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R—CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen.

In some embodiments, the patient has received an epcoritamab-containing regimen. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used as neoadjuvant therapy. In some embodiments, the neoadjuvant therapy precedes surgery (e.g., surgical resection, such as partial surgical resection or complete, total, or full surgical resection). In some embodiments, the neoadjuvant therapy precedes radiation therapy.

In some embodiments, the neoadjuvant therapy precedes chemotherapy.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is an adjuvant therapy. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R—CHOP, G-CHOP, R-EPOCH, CVP, CVAD, R², R-CODOX-M, R-IVAC, DA-EPOCH-R, cell-based therapy, or two or more thereof.

In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer. In some embodiments, the adjuvant therapy follows surgery (e.g., surgical resection, such as partial surgical resection or complete, total, or full surgical resection). In some embodiments, the adjuvant therapy follows radiation therapy. In some embodiments, the adjuvant therapy follows chemotherapy.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is a maintenance therapy. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), a stem cell transplant, or a combination thereof. In some embodiments, the patient has received R—CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the patient has received a stem cell transplant. In some embodiments, the patient has received a cell-based therapy (e.g., CAR T therapy). In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer.

As used herein, “monotherapy”, when referring to a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, means that the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, anemia, or gastrointestinal symptoms, including nausea, diarrhea, and lack of appetite.

As used herein, “the subject has previously received one or more therapeutic agents or therapies for the cancer” means that the subject has been previously administered one or more therapeutic agents or therapies (e.g., anticancer agent or therapy) for the cancer other than a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1). In some embodiments, a lack of response, an inadequate response, or a discontinued response can be determined by the subject's physician.

As used herein, “the subject is treatment naive with respect to the cancer” means that the subject has not been previously administered one or more therapeutic agents or therapies for the cancer.

For any of the solid tumors described herein, the solid tumor can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

In some embodiments of any of the methods or uses described herein, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor, liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic lymphocytic leukemia (CLL), acute lymphocytic leukemia (ALL) (e.g., Philadelphia chromosome positive ALL, Philadelphia chromosome negative ALL), acute myeloid leukemia (AML) (e.g., acute promyelocytic leukemia (APL)), chronic myeloid leukemia (CML)), lymphoma (e.g., Hodgkin lymphoma (e.g., nodular lymphocyte predominant Hodgkin lymphoma (NLPHL)), non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL))), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, mesothelioma, non-small cell lung cancer (NSCLC)), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma), thymus cancer (e.g., thymoma), or a combination thereof.

In some embodiments, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, pheochromocytoma, paraganglioma), thyroid cancer (e.g., papillary thyroid cancer)), eye cancer (e.g., uveal cancer (e.g., uveal melanoma)), gastrointestinal cancer (e.g., bile duct cancer (e.g., cholangiocarcinoma), colorectal cancer (e.g., colon adenocarcinoma, rectal adenocarcinoma, mucinous adenocarcinoma, mucinous carcinoma), esophageal cancer (e.g., esophageal adenocarcinoma), liver cancer (e.g., hepatocellular carcinoma), pancreatic cancer (e.g., pancreatic adenocarcinoma), or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), or testicular cancer (e.g., testicular germ cell tumors)), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), or uterine cancer (e.g., uterine carcinosarcoma, uterine endometrioid carcinoma, uterine serous carcinoma, uterine papillary serous carcinoma, uterine corpus endometrial carcinoma)), head and neck cancer (e.g., head and neck squamous cell carcinoma), hematological cancer (e.g., leukemia (e.g., chronic lymphocytic leukemia (CLL), acute lymphocytic leukemia (ALL) (e.g., B-cell lineage ALL (B-ALL), Philadelphia chromosome positive ALL (e.g., Philadelphia chromosome positive B-ALL), Philadelphia chromosome negative ALL (e.g., Philadelphia chromosome negative B-ALL)), acute myeloid leukemia (AML), chronic myeloid leukemia (CML)) or lymphoma (e.g., Hodgkin lymphoma (e.g., nodular lymphocyte predominant Hodgkin lymphoma (NLPHL)), non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL)))), respiratory cancer (e.g., lung cancer (e.g., lung squamous cell carcinoma, lung adenocarcinoma, mesothelioma, non-small cell lung cancer (NSCLC))), sarcoma (e.g., leiomyosarcoma, myxofibrosarcoma), skin cancer (e.g., melanoma), thymus cancer (e.g., thymoma), or a combination thereof.

In some embodiments, the cancer is a hematological cancer (e.g., a lymphoma (e.g., diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL), Burkitt lymphoma (BL), mantle cell lymphoma (MCL)) or a leukemia (e.g., chronic lymphocytic leukemia (CLL), acute lymphocytic leukemia (ALL) (e.g., B-cell lineage ALL (B-ALL), Philadelphia chromosome positive ALL (e.g., Philadelphia chromosome positive B-ALL), Philadelphia chromosome negative ALL (e.g., Philadelphia chromosome negative B-ALL)), chronic myeloid leukemia (CML))), breast cancer, gastrointestinal cancer, brain cancer (e.g., glioblastoma) or lung cancer (e.g., NSCLC). In some embodiments, the cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML). In some embodiments, the cancer is hematological cancer is DLBCL, FL, MCL, BL, PTCL, or ALL (e.g., B-ALL). In some embodiments, the cancer is FL or DLBCL. In some embodiments, the cancer is DLBCL, FL, MCL, or ALL (e.g., B-ALL). See, e.g., Leeman-Neill and Bhagat, Expert Opinion on Therapeutic Targets 22.2 (2018): 143-152; Mlynarczyk and Melnick. Immunological Reviews 288.1 (2019): 214-239; Hurtz, Christian, et al., Journal of Experimental Medicine 208.11 (2011): 2163-2174; Deb, Dhruba, et al. Cancer Research 77.11 (2017): 3070-3081; Cardenas, Mariano G., et al., Clinical Cancer Research 23.4 (2017): 885-893; Walker, Sarah R., et al., Oncogene 34.9 (2015): 1073-1082; International Publication Nos. WO 2021/080950, WO 2021/077010, and WO 2022/221673.

In some embodiments, the cancer is non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH)), or small lymphocytic lymphoma (SLL))). In some embodiments, the non-Hodgkin lymphoma is B-cell non-Hodgkin lymphoma. In some embodiments, the non-Hodgkin lymphoma is CD20-positive. In some embodiments, the non-Hodgkin lymphoma is CD20-positive B-cell non-Hodgkin lymphoma. In some embodiments, the patient has not been previously treated for the non-Hodgkin lymphoma. In some embodiments, the patient has previously received chemotherapy. In some embodiments, the patient has been previously treated with rituximab or obinutuzumab as a monotherapy or in combination with an additional therapy or therapeutic agent.

In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with R—CHOP (RITUXAN® (rituximab), cyclophosphamide, hydroxydaunorubicin, vincristine, and prednisone), or G-CHOP (GAZYVA® (obinutuzumab)), cyclophosphamide, hydroxydaunorubicin, vincristine, and prednisone). In some cases, the patient has previously been treated with etoposide and R—CHOP (called R-EPOCH). In some cases, the patient has been previously treated with R—CHOP combined with lenalidomide, venetoclax, ibrutinib, acalabrutinib, obinutuzumab, polatuzumab, pembrolizumab, durvalumab, or mosunetuzumab. In some embodiments, the patient has been previously treated with cyclophosphamide, vincristine, and prednisone (CVP), with or without rituximab or obinutuzumab. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the non-Hodgkin lymphoma is non-progressing (including stable disease) non-Hodgkin lymphoma. In some embodiments, the non-Hodgkin lymphoma is relapsed or refractory non-Hodgkin lymphoma. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, cancer is a non-Hodgkin lymphoma, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy.

In some embodiments, the cancer is DLBCL. In some embodiments, the DLBCL is characterized by a BCL2 translocation, a BCL6 translocation, a CD79B mutation (e.g., H225Y, A205D, Y196del, Y196F, Y196D, Y207X, Y196N, A205fs, Y196S, Y196H, A205fs, T206fs, H194-E197delinsQ, E197G, K219T, E192fs, or Y196C), an EZH2 mutation (e.g., a Y646F, Y646N, A682G, or A692V mutation), a MYC translocation, a MYD88 mutation (e.g., a L265P mutation), a NOTCH]mutation (e.g., Q2394X, Q2501X, Q2459X, Y2490X, G2427fs, Q2444X, P2514fs, or P2517S), a NOTCH2 mutation (e.g., Q2285K, 52136fs, Q2361X, P2288fs, L2415fs, G2410fs, Q2409X, S2388X, I2304fs, Q2364X, Q2360fs, S2395X, E2261fs, M2267fs, Q2285fs, R2400X, P2303fs, Q2285fs, A2273fs, K2133fs, Q2389X, E2399X, E2290X, Q2325X, Y2340X, Y2392X, or E2411fs), a TP53 mutation (e.g., R181C, E336A, R248W, P98fs, P152L, R280I, S149fs, P151H, G245D, Y236D, S127F, A161T, D148fs, M246I, Y126C, H179R, A159P, C238G, L93fs, Y220C, R283fs, G244D, G245S, E171X, R209X, T155-R156dup, E271K, R306X, G105D, L93fs, G262V, W53X, G244V, H214Y, R282W, R337C, Q331fs, R273G, R273C, C176Y, S215R, R213Q, I195T, G245R, I232T, R175H, Y126D, R273H, R196X, Y205C, C141Y, C229X, Y126N, P278S, P151S, Y236H, R282G, Y103X, V216M, G244S, G266E, V173A, V173fs, I254S, T125M, R342X, P152fs, Y205D, V274L, L257P, C135Y, C176R, Y234N, R248Q, G244R, Y234H, R248G, M237I, R213X, E258D, V173M, L252-I254del, L252I, Y234C, or C176F), 17p deletion, 18q gain, or a combination thereof. In some embodiments, the DLBCL has a BCL6 rearrangement, a NOTCH2 mutation (e.g., Q2285K, 52136fs, Q2361X, P2288fs, L2415fs, G2410fs, Q2409X, S2388X, I2304fs, Q2364X, Q2360fs, S2395X, E2261fs, M2267fs, Q2285fs, R2400X, P2303fs, Q2285fs, A2273fs, K2133fs, Q2389X, E2399X, E2290X, Q2325X, Y2340X, Y2392X, E241ifs), or a combination thereof. In some embodiments, the DLBCL is DLBCL having a germinal center B cell (GCB) cell of origin. In some embodiments, the DLBCL is a BN2-type DLBCL (e.g., having a BCL6 rearrangement and/or a NOTCH2 mutation). In some embodiments, the DLBCL is an EZB-type DLBCL (e.g., having an EZH2 mutation and/or a BCL2 translocation). In some embodiments, the DLBCL is a C1 genetic cluster DLBCL (e.g., having a BCL6 rearrangement and/or a NOTCH2 mutation). See, e.g., Schmitz, Roland, et al. New England Journal of Medicine 378.15 (2018): 1396-1407; Chapuy, Bjoern, et al. Nature Medicine 24.5 (2018): 679-690 for additional description of these classifications.

In some embodiments, the cancer is a FL. In some embodiments, the FL has a BCL2 translocation (e.g., a t(14;18) translocation). In some embodiments, the FL has an EZH2 mutation (e.g., a Y646F, Y646N, A682G, or A692V mutation). See, e.g., Kridel, Robert, Laurie H. Sehn, and Randy D. Gascoyne. The Journal of Clinical Investigation 122.10 (2012): 3424-3431. In some embodiments, the cancer is a FL, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy.

In some embodiments, the cancer is a B-ALL. In some embodiments, the B-ALL has an MLL rearrangement (e.g., an MLL-Af4 fusion, an MLL-Af6 fusion, an MLL-Af9 fusion, an MLL-ENL fusion, or an MLL-PTD fusion), is pre-B cell receptor positive (Pre-BCR+), has the Philadelphia chromosome, is Philadelphia chromosome-like, is dependent on R^{a5} signaling, has a BCL2 amplification, has a JAK2 mutation (with or without high cytokine receptor-like factor 2 (CRLF2) expression), or a combination thereof. See, e.g., Knight, Thomas, and Julie Anne Elizabeth Irving. Frontiers in Oncology 4 (2014): 160; Geng, Huimin, et al. Cancer Cell 27.3 (2015): 409-425; Jain, Nitin, et al. Blood, 129.5 (2017): 572-581; and Hurtz, Christian, et al. Genes & Development 33.17-18 (2019): 1265-1279. In some embodiments, the cancer is a B-ALL, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used in the treatment of patient having a B-ALL. In some embodiments, the B-ALL is a relapsed or refractory B-ALL after two or more lines of systemic therapy.

In some embodiments, the patient has previously been treated with another anticancer agent, a chemotherapeutic agent, surgery, radiation, a multi-kinase inhibitor, or a combination thereof.

In some embodiments, the cancer is a DLBCL. In some embodiments, the patient has not been previously treated for the DLBCL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with lenalidomide in combination with rituximab or obinutuzumab. In some embodiments, the patient has previously been treated with cyclophosphamide, vincristine and prednisone (CVP), optionally in combination with rituximab or obinutuzumab. In some embodiments, the patient has previously been treated with R—CHOP (RITUXAN® (rituximab), cyclophosphamide, hydroxydaunorubicin, vincristine, and prednisone), or G-CHOP (GAZYVA® (obinutuzumab)), cyclophosphamide, hydroxydaunorubicin, vincristine, and prednisone). In some cases, the patient has previously been treated with etoposide and R—CHOP (called R-EPOCH). In some cases, the patient has been previously treated with R—CHOP combined with lenalidomide, venetoclax, ibrutinib, acalabrutinib, obinutuzumab, polatuzumab, pembrolizumab, durvalumab, or mosunetuzumab. In some embodiments, the patient has been previously treated with a rituximab-containing regimen. In some embodiments, the patient has been previously treated with an obinutuzumab-containing regimen. In some embodiments, the patient has been previously treated with a mosunetuzumab-containing regimen. In some embodiments, the patient has been previously treated with an epcoritamab-containing regimen. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the DLBCL is non-progressing (including stable disease) DLBCL. In some embodiments, the DLBCL is relapsed or refractory DLBCL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a DLBCL, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a DLBCL.

In some embodiments, the DLBCL is a relapsed or refractory DLBCL after two or more lines of systemic therapy.

In some embodiments, the cancer is a FL. In some embodiments, the patient has not been previously treated for the FL. In some embodiments, the patient has previously received chemotherapy. In some embodiments, the patient has been previously treated with rituximab or obinutuzumab as a monotherapy or in combination with an additional therapy or therapeutic agent.

In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with lenalidomide in combination with rituximab or obinutuzumab (the combination with rituximab is sometimes called “R²”). In some embodiments, the patient has previously been treated with cyclophosphamide, vincristine and prednisone (CVP), optionally in combination with rituximab or obinutuzumab. In some embodiments, the patient has previously been treated with R—CHOP or G-CHOP. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the non-Hodgkin lymphoma is non-progressing (including stable disease) FL. In some embodiments, the FL is relapsed or refractory FL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a FL, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a FL. In some embodiments, the FL is a relapsed or refractory FL after two or more lines of systemic therapy.

In some embodiments, the cancer is a BL. In some embodiments, the patient has not been previously treated for the BL. In some embodiments, the patient has previously received chemotherapy. In some embodiments, the patient has been previously treated with rituximab or obinutuzumab as a monotherapy or in combination with an additional therapy or therapeutic agent.

In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with R—CHOP or G-CHOP. In some embodiments, the patient has previously been treated with rituximab, cyclophosphamide, vincristine, doxorubicin, and methotrexate (R-CODOX-M). In some embodiments, the patient has previously been treated with rituximab, ifosfamide, etoposide, and cytarabine (R-IVAC). In some embodiments, the patient has previously been treated with rituximab with dose-adjusted etoposide, prednisolone, vincristine, cyclophosphamide, and doxorubicin (DA-EPOCH-R). In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the BL is non-progressing (including stable disease) BL. In some embodiments, the BL is relapsed or refractory BL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a BL, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a BL. In some embodiments, the BL is a relapsed or refractory BL after two or more lines of systemic therapy.

In some embodiments, the cancer is a PTCL (e.g., PTCL with a T follicular helper phenotype (PTPCL-TFH), angioimmunoblastic T-cell lymphoma (AITL), or PTCL not otherwise specified (PTCL-NOS)). In some embodiments, the patient has not been previously treated for the PTCL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with lenalidomide in combination with rituximab or obinutuzumab (the combination with rituximab is sometimes called “R²”). In some embodiments, the patient has previously been treated with cyclophosphamide, vincristine and prednisone (CVP), optionally in combination with rituximab or obinutuzumab (R—CVP or G-CVP, respectively). In some embodiments, the patient has previously been treated with R—CHOP or G-CHOP. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the PTCL is non-progressing (including stable disease) PTCL. In some embodiments, the PTCL is relapsed or refractory PTCL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a PTCL, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient having a PTCL. In some embodiments, the PTCL is a relapsed or refractory PTCL after two or more lines of systemic therapy.

In some embodiments, the cancer is B-ALL. In some embodiments, the B-ALL is Philadelphia chromosome positive B-ALL. In some embodiments, the B-ALL is Philadelphia chromosome negative B-ALL. In some embodiments, the patient has previously received chemotherapy. In some embodiments, the patient has previously been treated with at least one cycle of induction, consolidation, intensification, and optional maintenance. In some cases, induction therapy can include an anthracycline, vincristine, a corticosteroid, and cyclophosphamide. In some embodiments, the anthracycline is doxorubicin. In some embodiments, the corticosteroid is dexamethasone. In some cases, the combination of doxorubicin, vincristine, dexamethasone, and cyclophosphamide is known as CVAD. In some embodiments, induction therapy can further include a tyrosine kinase inhibitor (e.g., a BCR-ABL inhibitor for patients with this fusion). In some embodiments, induction therapy can further include asparaginase (e.g., for pediatric patients). In some cases, consolidation therapy can include methotrexate, cytarabine, vincristine, 6-mercaptopurine, 6-thioguanine, cyclophosphamide, and etoposide. In some embodiments, consolidation therapy can further include a tyrosine kinase inhibitor (e.g., a BCR-ABL inhibitor for patients with this fusion). In some embodiments, consolidation therapy can further include asparaginase (e.g., for pediatric patients). In some cases, intensification therapy can include an anthracycline, vincristine, a corticosteroid, and cyclophosphamide. In some embodiments, intensification therapy can further include a tyrosine kinase inhibitor (e.g., a BCR-ABL inhibitor for patients with this fusion). In some embodiments, intensification therapy can further include asparaginase (e.g., for pediatric patients). Typically, pediatric and young adult regimens include higher cumulative doses of asparaginase and vincristine but may have lower cumulative doses of anthracycline and cyclophosphamide compared to adult regimens. In any of these cycle phases, an anti-CD20 immunotherapy (e.g., rituximab) can be added for patients expressing the CD20 protein on the cells. See, e.g., Muffly, Lori, and Emily Curran. Hematology 2014, the American Society of Hematology Education Program Book 2019.1 (2019): 17-23. In some embodiments, the patient has received one or more lines of systemic therapy. In some embodiments, the patient has received two or more lines of systemic therapy. In some embodiments, the B-ALL is relapsed or refractory B-ALL. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a B-ALL, and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a B-ALL. In some embodiments, the B-ALL is a relapsed or refractory B-ALL after two or more lines of systemic therapy.

Also provided herein is a method of treating a subject having a cancer, wherein the method comprises: administering a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as a monotherapy or in conjunction with a first anticancer agent to the subject who has been administered one or more doses of the first anticancer agent to the subject for a period of time.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as a monotherapy or in conjunction with the first anticancer agent to the subject.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as a monotherapy or in conjunction with a second anticancer agent to the subject.

In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.d. (once daily) to the subject. In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.d. (twice daily) to the subject. In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.d. (three times daily) to the subject. In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.i.d. (four times daily) to the subject. In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.o.d. (every other day) to the subject. In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.week (once weekly) to the subject. In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.w. (twice weekly) to the subject. In some embodiments of any of the methods of treating cancers provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.w. (three times weekly) to the subject.

BCL6 activity has also been implicated in autoimmunity. See, for example, Li, Qing, et al. European Journal of Immunology 50.4 (2020): 525-536; Pearce, Andrew C., et al. Journal of Biological Chemistry 297.2 (2021); Venkatadri, Rajkumar, et al. European Journal of Immunology 52.5 (2022): 825-834; Patel, Preeyam S., et al. Science Advances 8.25 (2022): eabol782; Ding, Shu, Yu Rao, and Qianjin Lu. Cellular & Molecular Immunology 19.7 (2022): 863-865. Accordingly, also provided herein is a method of treating an autoimmune condition in a subject in need thereof, the method comprising administering to the subject a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for the treatment of an autoimmune condition, for example, any of the autoimmune conditions provided herein.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a medicament for the treatment of an autoimmune condition, for example, any of the autoimmune conditions provided herein.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in the manufacture of a medicament for the treatment of an autoimmune condition, for example, any of the autoimmune conditions provided herein.

Provided herein is a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament for the treatment of an autoimmune condition, for example, any of the autoimmune conditions provided herein.

Provided herein is a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use in treating an autoimmune condition, for example, any of the autoimmune conditions provided herein.

In some embodiments, the autoimmune condition is acquired hemophilia, Addison's disease, ankylosing spondylitis, anti-neutrophil cytoplasmic antibody associated vasculitis (ANCA vasculitis), anti-synthetase syndrome, atherosclerosis, autoimmune hemolytic anemia, autoimmune hepatitis, autoimmune sclerosing cholangitis, autoimmune thyroiditis, autoimmune uveitis, Crohn's disease, dermatomyositis, diffuse scleroderma, Goodpasture's syndrome, graft-versus-host disease (GVHD) (e.g., chronic graft-versus-host disease (cGVHD)), Graves' disease, Guillain-Barre syndrome, Hashimoto's thyroiditis, Hughes' syndrome, IgG4-related disease, immune thrombocytopenic purpura (ITP), inflammatory bowel disease, limited scleroderma, multiple sclerosis, myasthenia gravis (MG), neuromyelitis optica spectrum disorders (NMOSD) (e.g., neuromyelitis optica (NMO)), pemphigoid, pemphigus, pernicious anemia, polymyositis, primary biliary cirrhosis, psoriasis, psoriatic arthritis, rheumatoid arthritis, seronegative spondyloarthropathies, Sjogren's syndrome, systemic lupus erythematosus, thrombocytopenic purpura, Type 1 diabetes, ulcerative colitis, vitiligo, or a combination thereof. In some embodiments, the autoimmune condition is rheumatoid arthritis, systemic lupus erythematosus, or a combination thereof. In some embodiments, the autoimmune condition is ANCA vasculitis, GVHD (e.g., cGVHD), myasthenia gravis, NMO, or a combination thereof. In some embodiments, the autoimmune condition is ANCA vasculitis, anti-synthetase syndrome, arthritis (e.g., rheumatoid arthritis or inflammatory arthritis), GVHD (e.g., cGVHD), IgG4-RD, lupus (e.g., lupus erythematosus), ITP, MG (e.g., muscle-specific tyrosine kinase (MuSK) positive MG), MS, NMOSD (e.g., NMO), pemphigus (e.g., pemphigus vulgaris), Sjogren's syndrome, or a combination thereof. In some embodiments, the autoimmune condition is ANCA vasculitis, anti-synthetase syndrome, GVHD (e.g., cGVHD), TIP, MG (e.g., muscle-specific tyrosine kinase (MuSK) positive MG), NMOSD (e.g., NMO), pemphigus, or a combination thereof. In some embodiments, the autoimmune condition is arthritis (e.g., rheumatoid arthritis or inflammatory arthritis), GVHD (e.g., cGVHD), IgG4-RD, lupus (e.g., lupus erythematosus), MG (e.g., muscle-specific tyrosine kinase (MuSK) positive MG), MS, NMOSD (e.g., NMO), pemphigus (e.g., pemphigus vulgaris), Sjogren's syndrome, or a combination thereof. See, e.g., Pearce, Andrew C., et al. Journal of Biological Chemistry 297.2 (2021); Ding, Shu, Yu Rao, and Qianjin Lu, Cellular & Molecular Immunology (2022): 1-3; Lee, Dennis S W, Olga L. Rojas, and Jennifer L. Gommerman, Nature Reviews Drug Discovery 20.3 (2021): 179-199; and International Publication Nos. WO 2020/014599; WO 2021/074620.

In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.d. (once daily) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.d. (twice daily) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.d. (three times daily) to the subject.

In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.i.d. (four times daily) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.o.d. (every other day) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.week (once weekly) to the subject.

In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.w. (twice weekly) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.w. (three times weekly) to the subject.

Also provided herein is a method of treating a lymphoproliferative disorder in a subject in need thereof, the method comprising administering to the subject a therapeutically effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for the treatment of a lymphoproliferative disorder, for example, any of the lymphoproliferative disorders provided herein.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a medicament for the treatment of a lymphoproliferative disorder, for example, any of the lymphoproliferative disorders provided herein.

Provided herein is use of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in the manufacture of a medicament for the treatment of a lymphoproliferative disorder, for example, any of the lymphoproliferative disorders provided herein.

Provided herein is a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament for the treatment of a lymphoproliferative disorder, for example, any of the lymphoproliferative disorders provided herein.

Provided herein is a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use in treating a lymphoproliferative disorder, for example, any of the lymphoproliferative disorders provided herein.

In some embodiments, the lymphoproliferative disorder is Epstein-Barr Virus-associated lymphoproliferative disorder.

Also provided is a method for modulating (e.g., decreasing) BCL6 protein activity in a cell, comprising contacting the cell with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject. In some embodiments, the cell is a cancer cell. In some embodiments, the cell is a mammalian cell. In some embodiments, the cell is a mammalian cancer cell. In some embodiments, the cancer cell is any cancer as described herein.

As used herein, the term “contacting” refers to the bringing together of indicated moieties in an in vitro system or an in vivo system. For example, “contacting” a cell with a compound provided herein includes the administration of a compound provided herein to the cell, in vitro or in vivo, including, for example, introducing a compound provided herein into a sample containing cells (e.g., grown in culture or derived from a patient), an organoid, or an organism (e.g., an animal (e.g., an animal bearing a tumor), or a human).

Also provided is a method of modulating (e.g., decreasing) the level of BCL6 protein in a cell, comprising contacting the cell with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the level of BCL6 protein is decreased by at least 30% (e.g., at least 40%, at least 50%, at least 60%, at least 70%, at least 80%, at least 90%, at least 95%, at least 97%, or at least 99%) compared to a cell not contacted with the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro.

In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject having a cell having a BCL6 protein.

In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of inducing ubiquitination of a BCL6 protein in a cell, comprising contacting the cell with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject having a cell having a BCL6 protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of forming a ternary complex comprising a BCL6 protein, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and a CRBN protein or fragment thereof in a cell, comprising contacting the cell with a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro.

In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject having a cell having a BCL6 protein.

In some embodiments, the cell is a cancer cell. In some embodiments, the cell is a mammalian cell. In some embodiments, the cell is a mammalian cancer cell. In some embodiments, the cancer cell is any cancer as described herein.

Also provided herein is a method for inducing degradation of a BCL6 protein in a mammalian cell, the method comprising contacting the mammalian cell with an effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Also provided herein is a method of inhibiting cell proliferation, in vitro or in vivo, the method comprising contacting a cell with an effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof as defined herein.

Further provided herein is a method of increasing cell death, in vitro or in vivo, the method comprising contacting a cell with an effective amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof as defined herein. Also provided herein is a method of increasing tumor cell death in a subject. The method comprises administering to the subject a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in an amount effective to increase tumor cell death.

When employed as pharmaceuticals, the compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, can be administered in the form of pharmaceutical compositions as described herein.

### Combinations

In any of the indications described herein, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be used as a monotherapy. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be used prior to administration of an additional therapeutic agent or additional therapy. For example, a subject in need thereof can be administered one or more doses of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, for a period of time and then undergo at least partial resection of the tumor. In some embodiments, the treatment with one or more doses of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, reduces the size of the tumor (e.g., the tumor burden) prior to the at least partial resection of the tumor.

In some embodiments, a subject in need thereof can be administered one or more doses of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (T-bb) (e.g., Formula (T-bb-1) or (I-bb-2)), or Formula (I-b) (e.g., Formula (I-b-1) or (I-b-2))) or Formula (II), or a pharmaceutically acceptable salt thereof, for a period of time and undergo one or more rounds of radiation therapy. In some embodiments, the treatment with one or more doses of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (T-bb) (e.g., Formula (T-bb-1) or (I-bb-2)), or Formula (T-b) (e.g., Formula (I-b-1) or (I-b-2))) or Formula (II), or a pharmaceutically acceptable salt thereof, reduces the size of the tumor (e.g., the tumor burden) prior to the one or more rounds of radiation therapy.

In some embodiments of any the methods described herein, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (T-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered in combination with a therapeutically effective amount of at least one additional therapeutic (e.g., chemotherapeutic) agent.

Non-limiting examples of additional therapeutic agents include: RAS pathway targeted therapeutic agents (e.g., R^{a5}/RAF/MEK/PI3K pathway inhibitors, (e.g., R^{a5} inhibitors, KRas-targeted therapeutic agents, SOS1 inhibitors, SOS1/R^{a5} protein-protein interaction inhibitors, SHP2 inhibitors, PI3K-AKT-mTOR pathway inhibitors)), kinase-targeted therapeutics (e.g., MEK inhibitors, ERK inhibitors, Raf inhibitors (e.g., BRaf inhibitors), PI3K inhibitors, AKT inhibitors, BTK inhibitors, mTOR inhibitors, CDK4/5 inhibitors, CDK4/6 inhibitors, MET inhibitors, JAK inhibitors (e.g., JAK2 inhibitors), FAK inhibitors, ErbB family inhibitors (e.g., EGFR inhibitors, Her2 inhibitors), Src inhibitors), menin inhibitors, mTORC1 inhibitors, YAP inhibitors, proteasome inhibitors, farnesyl transferase inhibitors, HSP90 inhibitors, PTEN inhibitors, inhibitors of the polycomb repressive complex 2 (PRC2) (e.g., EZH1/2 or EZH2 inhibitors), signal transduction pathway inhibitors, checkpoint inhibitors, modulators of the apoptosis pathway (e.g., BCL-2 inhibitors, BCL-X_{L} inhibitors), XPO1 inhibitors, steroids, chemotherapeutics, angiogenesis-targeted therapies, immune-targeted agents including immunomodulatory imide drugs (sometimes called “IMiDs” or “CELMoDs”), immunotherapy (e.g., anti-PD1, anti-PD-L1, anti-CD19, anti-CD20, anti-CD22, anti-CD3, anti-CD30, anti-CD79B, or anti-CD47 therapies, including antibodies (e.g., single-targeted antibodies targeting one or more of PDi, PD-L1, CD19, CD20, CD22, CD3, CD30, CD79B, or CD47, bispecific antibodies (including bispecific T cell engagers (BiTEs)) targeting one or more of PD1, PD-L1, CD19, CD20, CD22, CD3, CD30, CD79B, or CD47, and antibody-drug conjugates (ADCs) incorporating one or more of PDi, PD-L1, CD19, CD20, CD22, CD3, CD30, CD79B, or CD47 antibodies) or antigen-binding fragments thereof, a PD-1 inhibitor, or a PD-L1 inhibitor), cell-based therapeutics (e.g., adoptive cell therapy (e.g., CAR T therapy, cytokine-induced killer cells (CIKs), natural killer cells (e.g., CAR-modified NK cells)) or antibody-armed cell therapy), and radiotherapy.

As used herein, a biosimilar antibody refers to an antibody or antigen-binding fragment that has the same primary amino acid sequence as compared to a reference antibody and optionally, may have detectable differences in post-translation modifications (e.g., glycosylation and/or phosphorylation) as compared to the reference antibody (e.g., a different glycoform).

In some embodiments, the additional therapeutic agent is a PI3K inhibitor, an Abl inhibitor (e.g., a BCR-Abl inhibitor), a BTK inhibitor, a JAK inhibitor (e.g., a JAK2 inhibitor), a BRaf inhibitor, a MEK inhibitor, a menin inhibitor, a BCL-2 inhibitor, a BCL-X_{L} inhibitor, an MCL-1 inhibitor, an XPO1 inhibitor, an inhibitor of the polycomb repressive complex 2 (e.g., an EZH1/2 or EZH2 inhibitor), an immunomodulatory imide drug, a steroid, anti-CD19 therapy, anti-CD20 therapy, anti-CD3 therapy, chemotherapy, or a combination thereof.

Without being bound by any particular theory, it is believed that targeting BCL6 can result in the induction of genes that it typically represses, such as BCL2. In some embodiments, the additional therapeutic agent is a BCL-2 inhibitor (e.g., venetoclax, navitoclax, lisaftoclax, obatoclax, pelcitoclax, AZD-0466, BGB-11417, UBX-1325, UBX-1967, ZN-d5, oblimersen (e.g., oblimersen sodium), or beclanorsen).

In some embodiments, the PI3K inhibitor is alpelisib (BYL719), amdizalisib, apitolisib (GDC-0980), bimiralisib, buparlisib (BKM120), copanlisib (ALIQOPA™, BAY80-6946) (e.g., copanlisib dihydrochloride or a hydrate of copanlisib dihydrochloride), dactolisib (NVP-BEZ235, BEZ-235), dezapelisib, dordaviprone, duvelisib (e.g., a hydrate of duvelisib), eganelisib, fimepinostat, gedatolisib (PF-05212384, PKI-587), idelalisib, inavolisib, leniolisib (e.g., leniolisib phosphate), linperlisib, omipalisib (GSK2126458, GSK458), parsaclisib, pictilisib (GDC-0941), pilaralisib (XL147, SAR245408), paxalisib, rigosertib, risovalisib, seletalisib, serabelisib (TAK-117, MLN1117, INK 1117), sonolisib (PX-866), taselisib (GDC-0032, RG7604), umbralisib (e.g., umbralisib tosylate), voxtalisib (XL756, SAR245409), wortmannin, zandelisib, AMG 511, AMG319, ASN003, AZD8835, BGT-226 (NVP-BGT226), CH5132799, CUDC-907, GDC-0077, GDC-0084 (RG7666), GS-9820, GSK1059615, GSK2636771, KIN-193 (AZD-6428), LY2023414, LY294002, PF-04691502, PI-103, PKI-402, PQR309, SAR260301, SF1126, SHC-014748-M, TQ-B-3525, VS-5584 (SB2343), WX-037, XL-765, ZSTK474, or a combination thereof. In some embodiments, the PI3K inhibitor is alpelisib, amdizalisib, apitolisib, bimiralisib, buparlisib, copanlisib (e.g., copanlisib dihydrochloride or a hydrate of copanlisib dihydrochloride), dactolisib, dezapelisib, dordaviprone, duvelisib (e.g., a hydrate of duvelisib), eganelisib, fimepinostat, gedatolisib, idelalisib, inavolisib, leniolisib (e.g., leniolisib phosphate), linperlisib, parsaclisib, paxalisib, risovalisib, seletalisib, serabelisib, sonolisib, tenalisib, umbralisib (e.g., umbralisib tosylate), zandelisib, PF-04691502, SHC-014748-M, TQ-B-3525, or a combination thereof.

In some embodiments, the Abl inhibitor (e.g., BCR-Abl inhibitor) is asciminib (e.g., asciminib hydrochloride), bafetinib, bosutinib (e.g., bosutinib monohydrate), danusertib, dasatinib (e.g., dasatinib monohydrate), flumatinib (e.g., flumatinib mesylate), imatinib (e.g., imatinib mesylate), nilotinib (e.g., nilotinib monochloride monohydrate), olverembatinib (e.g., olverembatinib mesylate), ponatinib (e.g., ponatinib hydrochloride), radotinib (e.g., radotinib dihydrochloride), ruserontinib, vandetanib, AN-019, AT-9283, IkT-148009, NPB-001-056, or a combination thereof.

In some embodiments, the cancer is a B-ALL, and the additional therapy or therapeutic agent is an Abl inhibitor. In some embodiments, the cancer is a Philadelphia chromosome positive B-ALL and the additional therapy or therapeutic agent is an Abl inhibitor. In some embodiments, the cancer is a Philadelphia chromosome-like B-ALL, and the additional therapy or therapeutic agent is an Abl inhibitor. In some embodiments, the Abl inhibitor is selected from the group consisting of imatinib, dasatinib, ponatinib, or a combination thereof.

In some embodiments, the BTK inhibitor is abivertinib, acalabrutinib, atuzabrutinib, branebrutinib, dasatinib (e.g., dasatinib monohydrate), edralbrutinib (SHR-1459), elsubrutinib, evobrutinib, fenebrutinib, ibrutinib, luxeptinib, nemtabrutinib, olafertinib, nemtabrutinib, orelabrutinib, pirtobrutinib, remibrutinib, rilzabrutinib, spebrutinib, sunvozertinib, tirabrutinib (e.g., tirabrutinib hydrochloride), tolebrutinib, vecabrutinib, zanubrutinib, AC-0058 (AC-0058TA), BMS-986142, CT-1530, DTRMWXHS-12, LY-3337641 (HM-71224), M-7583, TAS-5315, or a combination thereof. In some embodiments, the BTK inhibitor is abivertinib, acalabrutinib, atuzabrutinib, branebrutinib, dasatinib (e.g., dasatinib monohydrate), edralbrutinib, elsubrutinib, evobrutinib, fenebrutinib, ibrutinib, nemtabrutinib, orelabrutinib, pirtobrutinib, remibrutinib, rilzabrutinib, sunvozertinib, tirabrutinib (e.g., tirabrutinib hydrochloride), tolebrutinib, zanubrutinib, AC-0058, BMS-986142, DTRMWXHS-12, LY-3337641, TAS-5315, or a combination thereof.

In some embodiments, the cancer is a B-ALL, and the additional therapy or therapeutic agent is a BTK inhibitor. In some embodiments, the cancer is a pre-BCR+B-ALL, and the additional therapy or therapeutic agent is a BTK inhibitor. In some embodiments, the cancer is a B-ALL dependent on R^{a5} signaling, and the additional therapy or therapeutic agent is a BTK inhibitor. In some embodiments, the BTK inhibitor is ibrutinib or acalabrutinib.

In some embodiments, the JAK inhibitor is abrocitinib, baricitinib, brepocitinib, decernotinib, delgocitinib, deuruxolitinib, elsubrutinib, fedratinib (e.g., fedratinib dihydrochloride monohydrate), filgotinib (e.g., filgotinib maleate), gandotinib, gusacitinib, ilginatinib, itacitinib, ivarmacitinib, izencitinib, jaktinib, momelotinib, nezulcitinib, pacritinib (e.g., pacritinib citrate), peficitinib (e.g., peficitinib hydrobromide), povorcitinib (INCB-54707), ropsacitinib, ruxolitinib (e.g., ruxolitinib phosphate), solcitinib, tasocitinib (e.g., tofacitinib citrate), tinengotinib, upadacitinib (e.g., upadacitinib hydrate), zasocitinib, AGA-201, ATI-1777, ATI-501, ESK-001, GLPG-3667, INCB-52793, LNK-01001, LNK-01003, R-348, TD-8236, TLL-018, TQ-05105, VTX-958, or a combination thereof. In some embodiments, the JAK inhibitor is abrocitinib, baricitinib, brepocitinib, decernotinib, delgocitinib, deuruxolitinib, fedratinib (e.g., fedratinib dihydrochloride monohydrate), filgotinib (e.g., filgotinib maleate), gandotinib, gusacitinib, ilginatinib, itacitinib, ivarmacitinib, izencitinib, jaktinib, momelotinib, nezulcitinib, pacritinib (e.g., pacritinib citrate), peficitinib (e.g., peficitinib hydrobromide), povorcitinib (INCB-54707), ropsacitinib, ruxolitinib (e.g., ruxolitinib phosphate), solcitinib, tasocitinib (e.g., tofacitinib citrate), tinengotinib, upadacitinib (e.g., upadacitinib hydrate), zasocitinib, AGA-201, ATI-1777, ATI-501, ESK-001, GLPG-3667, LNK-01001, LNK-01003, R-348, TD-8236, TLL-018, TQ-05105, VTX-958, or a combination thereof.

In some embodiments, the cancer is a B-ALL, and the additional therapy or therapeutic agent is a JAK inhibitor. In some embodiments, the cancer is a JAK2 (e.g., JAK2^{R683G} or JAK2^{I682}F) mutant B-ALL, and the additional therapy or therapeutic agent is a JAK inhibitor. In some embodiments, the cancer is a JAK2 (e.g., JAK2^{R683G} or JAK2^{I682}F) mutant B-ALL with high CRLF2 expression, and the additional therapy or therapeutic agent is a JAK inhibitor and a BCL-2 inhibitor (e.g., venetoclax).

In some embodiments, the BRaf inhibitor is avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, PLX-4720, or a combination thereof.

In some embodiments, the MEK inhibitor is avutometinib (RO5126766), binimetinib (MEKTOVI®, MEK162), cobimetinib (e.g., cobimetinib fumarate, COTELLIC®), mirdametinib (PD0325901), pimasertib (MSC1936369B), refametinib, selumetinib (e.g., selumetinib sulfate, AZD6244), trametinib (e.g., trametinib dimethyl sulfoxide, GSK-1120212), zapnometinib, CI1040 (PD184352), CS3006, FCN-159, GSK-1120212, NFX-179, PD98059, SHR7390, TAK-733, WX-554, or a combination thereof. In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide, GSK-1120212), zapnometinib, FCN-159, NFX-179, TAK-733, or a combination thereof.

In some embodiments, the menin inhibitor is revumenib (e.g., revumenib fumarate), ziftomenib, BMF-219, DS-1594, JNJ-6617, or a combination thereof.

In some embodiments, the cancer is a B-ALL, and the additional therapy or therapeutic agent is a menin inhibitor. In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is a menin inhibitor.

In some embodiments, the BCL-2 inhibitor is lisaftoclax, navitoclax, obatoclax, venetoclax, oblimersen (e.g., oblimersen sodium), beclanorsen, AZD-0466, BGB-11417, UBX-1325, UBX-1967, ZN-d5, or a combination thereof. In some embodiments, the BCL-2 inhibitor is lisaftoclax, navitoclax, obatoclax, venetoclax, oblimersen (e.g., oblimersen sodium), beclanorsen, or a combination thereof.

In some embodiments, the cancer is a B-ALL, and the additional therapy or therapeutic agent is a BCL-2 inhibitor. In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is a BCL-2 inhibitor. In some embodiments, the cancer is a BCL2 amplified B-ALL, and the additional therapy or therapeutic agent is a BCL-2 inhibitor. In some embodiments, the BCL-2 inhibitor is venetoclax.

In some embodiments, the cancer is a DLBCL, and the additional therapy or therapeutic agent is a BCL-2 inhibitor. In some embodiments, the BCL-2 inhibitor is venetoclax.

In some embodiments, the cancer is a FL, and the additional therapy or therapeutic agent is a BCL-2 inhibitor. In some embodiments, the BCL-2 inhibitor is venetoclax.

In some embodiments, the cancer is an MCL, and the additional therapy or therapeutic agent is a BCL-2 inhibitor. In some embodiments, the BCL-2 inhibitor is venetoclax.

In some embodiments, the BCL-X_{L} inhibitor is lisaftoclax, navitoclax, obatoclax, pelcitoclax, mirzotamab clezutoclax, ABBV-155, APG-1252-12A, AZD-0466, DT-2216, PA-15227, UBX-1325, UBX-1967, XZ-739, 753-B, or a combination thereof. In some embodiments, the BCL-X_{L} inhibitor is lisaftoclax, navitoclax, obatoclax, or a combination thereof.

In some embodiments, the MCL-1 inhibitor is omacetaxine (e.g., omacetaxine mepesuccinate).

In some embodiments, the XPO1 inhibitor is eltanexor, felezonexor, selinexor, verdinexor, BIIB-100, JS-110, or a combination thereof. In some embodiments, the XPO1 inhibitor is selinexor.

In some embodiments, the inhibitor of the PRC2 is lirametostat, tazemetostat (e.g., tazemetostat hydrobromide), tulmimetostat (CPI-0209), valemetostat (e.g., valemetostat tosylate), EBI-2511, HH-2853, HM-97662, PF-6821497, SHR-2554, XNW-5004, or a combination thereof.

In some embodiments, the inhibitor of the PRC2 is an EZH1/2 inhibitor, an EZH2 inhibitor, or a combination thereof. Non-limiting examples of EZH2 and/or EZH1/2 inhibitors are described in International Publication Nos. WO 2011/140325, WO 2012/005805, WO 2012/050532, WO 2012/118812, WO 2012/142513, WO 2012/142504, WO 2013/049770, WO 2013/039988, WO 2013/067300, WO 2015/141616, WO 2017/084494, WO 2018/210296, WO 2018/210302, WO 2019/091450, WO 2019/204490, WO 2019/226491, WO 2020/063863, WO 2020/171606, WO 2020/228591, WO 2021/016414, WO 2021/063332, WO 2021/063340, WO 2021/180235, and WO 2022/035303.

In some embodiments, the cancer is a DLBCL, and the additional therapy or therapeutic agent is an inhibitor of the PRC2 (e.g., an EZH1/2 or EZH2 inhibitor). In some embodiments, the cancer is a B-ALL (e.g., Philadelphia chromosome positive B-ALL, Philadelphia chromosome negative B-ALL, or B-ALL with an MLL rearrangement (e.g., an MLL-Af4 fusion, an MLL-Af6 fusion, an MLL-Af9 fusion, an MLL-ENL fusion, or an MLL-PTD fusion)), and the additional therapy or therapeutic agent is an inhibitor of the PRC2. In some embodiments, the cancer is a FL, and the additional therapy or therapeutic agent is an inhibitor of the PRC2 (e.g., an EZH1/2 or EZH2 inhibitor). In some embodiments, the cancer is an MCL, and the additional therapy or therapeutic agent is an inhibitor of the PRC2 (e.g., an EZH1/2 or EZH2 inhibitor).

An exemplary wild-type human EZH2 sequence is shown below. This is one of several isoforms of EZH2, and it will be understood that residue numbering may change based on the reference isoform.

(UniParc ID UPI000006D77C):
SEQ ID NO: 1
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHRKC
NYSFHATPNTYKRKNTETALDNKPCGPQCYQHLEGAKEFAAALTAERIKT
PPKRPGGRRRGRLPNNSSRPSTPTINVLESKDTDSDREAGTETGGENNDK
EEEEKKDETSSSSEANSRCQTPIKMKPNIEPPENVEWSGAEASMFRVLIG
TYYDNFCAIARLIGTKTCRQVYEFRVKESSIIAPAPAEDVDTPPRKKKRK
HRLWAAHCRKIQLKKDGSSNHVYNYQPCDHPRQPCDSSCPCVIAQNFCEK
FCQCSSECQNRFPGCRCKAQCNTKQCPCYLAVRECDPDLCLTCGAADHWD
SKNVSCKNCSIQRGSKKHLLLAPSDVAGWGIFIKDPVQKNEFISEYCGEI
ISQDEADRRGKVYDKYMCSFLFNLNNDFVVDATRKGNKIRFANHSVNPNC
YAKVMMVNGDHRIGIFAKRAIQTGEELFFDYRYSQADALKYVGIEREMEI
P

In some embodiments, the steroid is dexamethasone, prednisone, or a combination thereof.

In some embodiments, the immunomodulatory imide drug is avadomide, lenalidomide, iberdomide, pomalidomide, thalidomide, CC-99282, or a combination thereof.

In some embodiments, the additional therapy or therapeutic agent is lenalidomide and rituximab or obinutuzumab.

In some embodiments, the anti-CD19 therapy is blinatumomab (e.g., BLINCYTO® (blinatumomab) or a biosimilar thereof), coltuximab ravtansine, inebilizumab (e.g., inebilizumab-cdon, or a biosimilar thereof), loncastuximab tesirine (e.g., loncastuximab tesirine-lpyl, or a biosimilar thereof), obexelimab, tafasitamab (e.g., tafasitamab-cxix, or a biosimilar thereof), dDT-2219, biosimilars thereof, or a combination thereof. In some embodiments, the anti-CD19 therapy is a bispecific antibody or antigen-binding fragment thereof (e.g., BLINCYTO® (blinatumomab) or a biosimilar thereof). In some embodiments, the anti-CD19 therapy is an anti-CD19 and anti-CD3 bispecific antibody or antigen-binding fragment thereof (e.g., BLINCYTO® (blinatumomab) or a biosimilar thereof). In some embodiments, the anti-CD19 therapy is an antibody-drug conjugate (e.g., coltuximab ravtansine, loncastuximab tesirine (e.g., loncastuximab tesirine-lpyl, or a biosimilar thereof)).

In some embodiments, the anti-CD20 therapy is divozilimab, epcoritamab (e.g., epcoritamab-bysp, or a biosimilar thereof), glofitamab (e.g., COLUMVI® (glofitamab), or a biosimilar thereof), ibritumomab tiuxetan (e.g., ZEVALIN® (ibritumomab tiuxetan), or a biosimilar thereof), mosunetuzumab (e.g., mosunetuzumab-axgb, or a biosimilar thereof), obinutuzumab (e.g., GAZYVA® (obinutuzumab), or a biosimilar thereof), ocrelizumab (e.g., OCREVUS® (ocrelizumab), or a biosimilar thereof), odronextamab, ofatumumab (e.g., ARZERRA® (ofatumumab), or a biosimilar thereof), plamotamab, rituximab (e.g., RITUXAN® (rituximab), or a biosimilar thereof (e.g., rituximab-abbs, rituximab-arrx, rituximab-pvvr, ACELLBIA® (rituximab), HALPRYZA® (rituximab), HANLIKON® (rituximab), RIXATHON® (rituximab), REDITUX™ (rituximab), Retuxira (rituximab), BI-695500, GB-241, Mabion-CD20, RTXM-83, SAIT-101), ublituximab (e.g., ublituximab-xiiy, or a biosimilar thereof), veltuzumab, zuberitamab, MIL-62, SCT-400, TQB-2303, biosimilars thereof, or a combination thereof. In some embodiments, the anti-CD20 therapy is a bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof. In some embodiments, the anti-CD20 therapy is an anti-CD20 and anti-CD3 bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof. In some embodiments, the anti-CD20 therapy is an antibody-drug conjugate (e.g., ibritumomab tiuxetan (e.g., ZEVALIN® (ibritumomab tiuxetan), or a biosimilar thereof).

In some embodiments, the additional therapy or therapeutic agent is rituximab. In some such embodiments, the combination of rituximab and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy. In some such embodiments, the cancer is a FL.

In some embodiments, the additional therapy or therapeutic agent is obinutuzumab. In some such embodiments, the combination of obinutuzumab and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy. In some such embodiments, the cancer is a FL.

In some embodiments, the cancer is a DLBCL, and the additional therapy or therapeutic agent is anti-CD20 therapy. In some embodiments, the anti-CD20 therapy is rituximab, obinutuzumab, or a combination thereof.

In some embodiments, the cancer is a FL, and the additional therapy or therapeutic agent is anti-CD20 therapy. In some embodiments, the anti-CD20 therapy is rituximab, obinutuzumab, or a combination thereof.

In some embodiments, the anti-CD22 therapy is an antibody-drug conjugate (e.g., inotuzumab ozogamicin.

In some embodiments, the cancer is a B-ALL, and the additional therapy or therapeutic agent is anti-CD22 therapy. In some embodiments, the anti-CD22 therapy is an anti-CD22 antibody-drug conjugate (e.g., inotuzumab ozogamicin, or a biosimilar thereof).

In some embodiments, the anti-CD3 therapy is blinatumomab (e.g., BLINCYTO® (blinatumomab), or a biosimilar thereof), catumaxomab, elranatamab, epcoritamab (e.g., epcoritamab-bysp, or a biosimilar thereof), ertumaxomab, glofitamab (e.g., COLUMVI® (glofitamab), or a biosimilar thereof), linvoseltamab, mosunetuzumab (e.g., mosunetuzumab-axgb, or a biosimilar thereof), odronextamab, otelixizumab, plamotamab, talquetamab, tarlatamab, tebentafusp (e.g., tebentafusp-tebn, or a biosimilar thereof), teclistamab (e.g., teclistamab-cqyv, or a biosimilar thereof), teplizumab (e.g., teplizumab-mzwv, or a biosimilar thereof), visilizumab, biosimilars thereof, or a combination thereof. In some embodiments, the anti-CD3 therapy is bispecific antibody or antigen-binding fragment thereof (e.g., blinatumomab (e.g., BLINCYTO® (blinatumomab), or a biosimilar thereof), catumaxomab, elranatamab, epcoritamab (e.g., epcoritamab-bysp, or a biosimilar thereof), ertumaxomab, glofitamab (e.g., COLUMVI® (glofitamab), or a biosimilar thereof), linvoseltamab, mosunetuzumab (e.g., mosunetuzumab-axgb, or a biosimilar thereof), plamotamab, odronextamab, talquetamab, tarlatamab, tebentafusp (e.g., tebentafusp-tebn, or a biosimilar thereof), teclistamab (e.g., teclistamab-cqyv, or a biosimilar thereof), biosimilars thereof, or a combination thereof. In some embodiments, the anti-CD3 therapy is an anti-CD3 and anti-CD19 bispecific antibody or antigen-binding fragment thereof (e.g., BLINCYTO® (blinatumomab) or a biosimilar thereof). In some embodiments, the anti-CD3 therapy is an anti-CD20 and anti-CD3 bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof.

In some embodiments, the anti-CD30 therapy is brentuximab, brentuximab vedotin, iratumumab, AFM-13, biosimilars thereof, or a combination thereof. In some embodiments, the anti-CD30 therapy is a bispecific antibody or antigen-binding fragment thereof (e.g., AFM-13). In some embodiments, the anti-CD30 therapy is an antibody-drug conjugate (e.g., brentuximab vedotin, or a biosimilar thereof).

In some embodiments, the anti-CD79B therapy is polatuzumab (e.g., polatuzumab vedotin (e.g., polatuzumab vedotin-piiq, or a biosimilar thereof)), MGD-010, RG-7986, biosimilars thereof, or a combination thereof. In some embodiments, the anti-CD79B therapy is polatuzumab (e.g., polatuzumab vedotin (e.g., polatuzumab vedotin-piiq, or a biosimilar thereof)), MGD-010, biosimilars thereof, or a combination thereof. In some embodiments, the anti-CD79B therapy is a bispecific antibody or antigen-binding fragment thereof (e.g., MGD-010). In some embodiments, the anti-CD79B therapy is an antibody-drug conjugate (e.g., polatuzumab vedotin (e.g., polatuzumab vedotin-piiq, or a biosimilar thereof)).

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, AZD7709, biosimilars thereof, or a combination thereof. In some embodiments, the anti-PD1 therapy is a bispecific antibody or antigen-binding fragment thereof (e.g., cadonilimab, ivonescimab, rilvegostomig, tebotelimab, volrustomig, vudalimab, AZD7709, HX-009, RG-6139, biosimilars thereof, or a combination thereof). In some embodiments, the anti-PD1 therapy is an anti-PD1 and anti-CD47 bispecific antibody or antigen-binding fragment thereof (e.g., HX-009, or a biosimilar thereof). In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., IMFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof. In some embodiments, the anti-PD-L1 therapy is a bispecific antibody or antigen-binding fragment thereof (e.g., erfonrilimab, BNT-311, biosimilars thereof, or a combination thereof).

In some embodiments, the PD-L1 inhibitor is INCB-086550.

In some embodiments, the anti-CD47 therapy is lemzoparlimab, letaplimab, magrolimab, 6MW-3211, AO-176, CPO-107, HX-009, TTI-621, TTI-622, biosimilars thereof, or a combination thereof. In some embodiments, the anti-CD47 therapy is lemzoparlimab, magrolimab, HX-009, TTI-621, TTI-622, biosimilars thereof, or a combination thereof. In some embodiments, the anti-anti-CD47 therapy is a bispecific antibody or antigen-binding fragment thereof (e.g., HX-009). In some embodiments, the anti-CD47 therapy is an anti-CD47 and anti-PD1 bispecific antibody or antigen-binding fragment thereof (e.g., HX-009, or a biosimilar thereof).

In some embodiments, the cell-based therapy is adoptive cell therapy (e.g., CAR T therapy, cytokine-induced killer cells (CIKs), natural killer cells (e.g., CAR-modified NK cells)) or antibody-armed cell therapy. In some embodiments, the cell-based therapy is CAR T therapy. In some embodiments, the cell-based therapy is axicabtagene ciloleucel (e.g., YESCARTA® (axicabtagene ciloleucel), or a biosimilar thereof), brexucabtagene autoleucel (e.g., TECARTUS® (brexucabtagene autoleucel), or a biosimilar thereof), inaticabtagene autoleucel, lisocabtagene maraleucel (e.g., BREYANZI® (lisocabtagene maraleucel), or a biosimilar thereof), rapcabtagene autoleucel, relmacabtagene autoleucel (e.g., CARTEYVA® (relmacabtagene autoleucel), or a biosimilar thereof), tisagenlecleucel (e.g., KYMRIAH® (tisagenlecleucel), or a biosimilar thereof), varnimcabtagene autoleucel (e.g., IMN-003A, or a biosimilar thereof), zamtocabtagene autoleucel, BZ-019, CD19CAR CTL, CTL-119, TAK-007, XLCART-001, biosimilars thereof, or a combination thereof.

In some embodiments, the additional therapy or therapeutic agent is chemotherapy. In some embodiments, the chemotherapy is CVAD, hyperCVAD (cyclophosphamide, vincristine, doxorubicin, and dexamethasone), CHOP, R—CHOP, G-CHOP, EPOCH, R-EPOCH, Pola-R—CHP (polatuzumab vedotin, rituximab, cyclophosphamide, doxorubicin, and prednisone), R-CODOX-M, R-IVAC, DA-EPOCH-R, CVP, R—CVP, G-CVP, CVD (cyclophosphamide, vincristine, dacarbazine, including mini-CVD), bendamustine with rituximab or obinutuzumab, methotrexate-cytarabine, vincristine (with or without steroids (e.g., dexamethasone)), nelarabine, a hypomethylating agent (e.g., azacitidine and/or decitabine), CALGB8811, or pediatric-inspired multi-agent chemotherapy (e.g., GRAALL-2003, COG AALL-0434, CCG-1961, CALGB 10403, or the DFCI regimen).

In some embodiments, the cancer is a B-ALL, and the additional therapy or therapeutic agent is chemotherapy (e.g., CVAD). In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is chemotherapy (e.g., CVAD). In some embodiments, the cancer is a Philadelphia chromosome positive B-ALL, and the additional therapy or therapeutic agent is chemotherapy (e.g., CVAD). In some embodiments, the cancer is a Philadelphia chromosome-like B-ALL, and the additional therapy or therapeutic agent is chemotherapy (e.g., CVAD). In some embodiments, the cancer is a pre-BCR+B-ALL, and the additional therapy or therapeutic agent is chemotherapy (e.g., CVAD). In some embodiments, the cancer is a B-ALL dependent on R^{a5} signaling, and the additional therapy or therapeutic agent is a chemotherapy (e.g., CVAD).

In some embodiments, the cancer is a FL, and the additional therapy or therapeutic agent is R—CHOP. In some such embodiments, the combination of R—CHOP and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of R—CHOP and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy. In some embodiments, the cancer is a DLBCL, and the additional therapy or therapeutic agent is R—CHOP. In some such embodiments, the combination of R—CHOP and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of R—CHOP and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy.

In some embodiments, the cancer is a DLBCL, and the additional therapy or therapeutic agent is R-EPOCH. In some such embodiments, the combination of R-EPOCH and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of R-EPOCH and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy.

In some embodiments, the cancer is a DLBCL, and the additional therapy or therapeutic agent is Pola-R—CHP. In some such embodiments, the combination of Pola-R—CHP and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of Pola-R—CHP and a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy.

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in combination with one or more steps of induction, consolidation, intensification, or maintenance in a chemotherapeutic regimen.

Also provided herein is a method of treating cancer, comprising administering to a subject in need thereof (a) a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and (b) an additional therapeutic agent, for simultaneous, separate or sequential use for the treatment of cancer, wherein the amounts of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are together effective in treating the cancer. In some embodiments, the method comprises administering (c) at least one pharmaceutically acceptable carrier.

In some such embodiments, the additional therapeutic agent is an inhibitor of the PRC2 (e.g., an EZH1/2 inhibitor or an EZH2 inhibitor (e.g., any of the EZH1/2 inhibitors or EZH2 inhibitors described herein)), and the cancer is a cancer having an EZH2 alteration. In an aspect of this embodiment, the EZH2 alteration is a mutation is at residue 27, residue 34, residue 59, residue 141, residue 162, residue 172, residue 197, residue 238, residue 239, residue 246, residue 395, residue 401, residue 452, residue 510, residue 516, residue 556, residue 583, residue 618, residue 644, residue 646, residue 682, residue 690, residue 692, residue 716, residue 732, residue 744, residue 745, or a combination thereof, relative to SEQ ID NO. 1. In another aspect of this embodiment, the EZH2 alteration is a translocation. In another aspect of this embodiment, the EZH2 mutation is R27*, R34*, E59*, Q141*, E162*, V172Cfs*11, E197Rfs*12, E238*, E239*, E246*, G395Efs*29, E401Kfs*22, E401*, Y452*, K510Yfs*3, X516-splice, S556*, R583*, X618-splice, S644*, Y646F, Y646N, Y646S, R690G, R690H, A692V, F716Lfs*24, X732-splice, I744Mfs*25, E745Afs*24, EZH2-AUTS2, EZH2-TMEM176B, GALNT11-EZH2, or a combination thereof. In another aspect of this embodiment, the EZH2 mutation is at residue 646, residue 682, or residue 692 relative to SEQ ID NO. 1. In another aspect of this embodiment, the EZH2 mutation is Y646F, Y646N, A682G, or A692V relative to SEQ ID NO: 1. In another aspect of this embodiment, the cancer is a lymphoma (e.g., FL or DLBCL) and the EZH2 mutation is Y646F, Y646N, A682G, or A692V relative to SEQ ID NO: 1.

These additional therapeutic agents may be administered with one or more doses of the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or pharmaceutical composition comprising a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as part of the same or separate dosage forms, via the same or different routes of administration, and/or on the same or different administration schedules according to standard pharmaceutical practice known to one skilled in the art. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are administered simultaneously as separate dosages. In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are administered as separate dosages simultaneously, separately, or sequentially in any order, in jointly therapeutically effective amounts, e.g., in daily or intermittent dosages. In some embodiments, the compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are administered simultaneously as a combined dosage. When administered simultaneously, the two agents can be administered as a single dosage form (e.g., a fixed dosage form) or as separate dosages (e.g., non-fixed dosage forms).

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, and refer to any animal, including mammals such as mice, rats, other rodents, rabbits, dogs, cats, swine, cattle, sheep, horses, primates, and humans. In some embodiments, the subject is a human.

In some embodiments, the subject has experienced and/or exhibited at least one symptom of the disease, disorder, or condition to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need thereof, is sufficient to (i) treat a disease, disorder, or condition, (ii) attenuate, ameliorate, or eliminate one or more symptoms of the particular disease, disorder, or condition, or (iii) delay the onset of one or more symptoms of the particular disease, disorder, or condition described herein. The amount of a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

### Pharmaceutical Compositions and Administration

### General

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-o-cyclodextrins, or other solubilized derivatives can also be used to enhance delivery of compounds described herein. Dosage forms or compositions containing a compound as described herein in the range of 0.005% to 100% with the balance made up from non-toxic excipient may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as described herein, or a pharmaceutical composition thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-a-1), (I-a-2), (I-a-3), (I-a-4), (I-a-5), or (I-a-6)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the compound described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein, or a pharmaceutical composition thereof, are suitable for local delivery to the digestive or GI tract by way of oral administration (e.g., solid or liquid dosage forms.).

Solid dosage forms for oral administration include capsules, tablets, pills, powders, and granules. In such solid dosage forms, the compound is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound, or pharmaceutically acceptable salt thereof, as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter.

These compositions can be sterilized by conventional, well-known sterilization techniques. For various oral dosage form excipients such as tablets and capsules sterility is not required. The USP/NF standard is usually sufficient.

In certain embodiments, solid oral dosage forms can include one or more components that chemically and/or structurally predispose the composition for delivery of the compounds to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Also provided herein are compounds useful in the preparation of compounds of Formula (I) (e.g., Formula (I-aa) (e.g., Formula (I-aa-1), (I-aa-2), (I-aa-3), (I-aa-4), (I-aa-5), or (I-aa-6)), Formula (I-a) (e.g., Formula (I-aa-1), (I-aa-2), (I-aa-3), (I-aa-4), (I-aa-5), or (I-aa-6)), Formula (I-bb) (e.g., Formula (I-bb-1) or (I-bb-2), or Formula (I-b) (e.g., Formula (I-b-1) or (I-b-2))) or Formula (II), or pharmaceutically acceptable salts thereof.

Provided herein are compounds of Formula (SI):

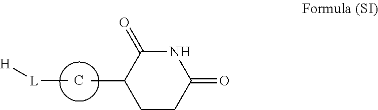

or salts thereof, wherein:
Ring C is selected from the group consisting of:

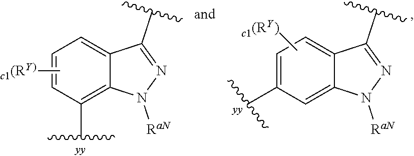

wherein: c1 is 0 or 1, R^{Y} is selected from the group consisting of halo (e.g., —F) and C₁₋₃ alkyl optionally substituted with 1-3 F, and R^{aN} is C₁₋₃ alkyl, and yy represents the point of attachment to L;
L is selected from the group consisting of:
   L^{A4}-L^{A1}-L^{A4}-_{bb};
   L^{A4}-L^{A1}-L^{A1}-L^{A4}-_{bb};
   L^{A4}-L^{A4}-_{bb};
   L^{A4}-C(═O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (SI), L is -L^{A4}-L^{A1}-L^{A4}-_{bb}.

In some embodiments of Formula (SI), L is -L^{A4}-L^{A}1-L^{A4}-_{bb}; and each L^{A4} contains 1-2 ring nitrogen atoms and no additional ring heteroatoms.

In some embodiments, compounds of Formula (SI) are compounds of Formula (SI-A) and Formula (SI—B):

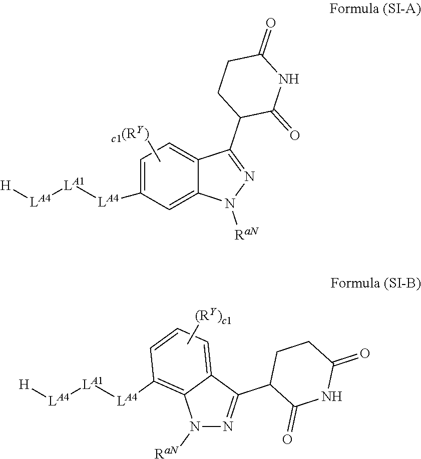

or salts thereof, wherein:
c1 is 0 or 1;
R^{Y} is selected from the group consisting of halo (e.g., —F) and C₁₋₃ alkyl optionally substituted with 1-3 F;
R^{aN} is C₁₋₃ alkyl;
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (SI-A) and Formula (SI—B), each L^{A4} contains 1-2 ring nitrogen atoms and no additional ring heteroatoms.

In some embodiments of Formula (SI-A) and Formula (SI—B), each L^{A4} is independently a monocyclic 4-6 membered nitrogen-containing heterocyclylene optionally substituted with 1-3 R^{a}.

In some embodiments of Formula (SI-A) and Formula (SI—B), one L^{A4} is a monocyclic 4-6 membered nitrogen-containing heterocyclylene optionally substituted with 1-3 R^{a}; and the other L^{A4} is a bicyclic spirocyclic 6-12 (e.g., 6-10) membered nitrogen-containing heterocyclylene optionally substituted with 1-3 R^{a}.

In some embodiments of Formula (SI-A) and Formula (SI—B), one L^{A4} is a monocyclic 4-6 membered nitrogen-containing heterocyclylene optionally substituted with 1-3 R^{a}; and
the other L^{A4} is a bicyclic bridged 6-12 (e.g., 7, 8, or 9) membered nitrogen-containing heterocyclylene optionally substituted with 1-3 R^{a}.

In some embodiments of Formula (SI-A) and Formula (SI—B), one L^{A4} is a monocyclic 4-6 membered nitrogen-containing heterocyclylene optionally substituted with 1-3 R^{a}; and the other L^{A4} is a bicyclic fused 6-12 (e.g., 6) membered nitrogen-containing heterocyclylene optionally substituted with 1-3 R^{a}.

For example, compounds of Formula (SI-A) and Formula (SI—B) can be

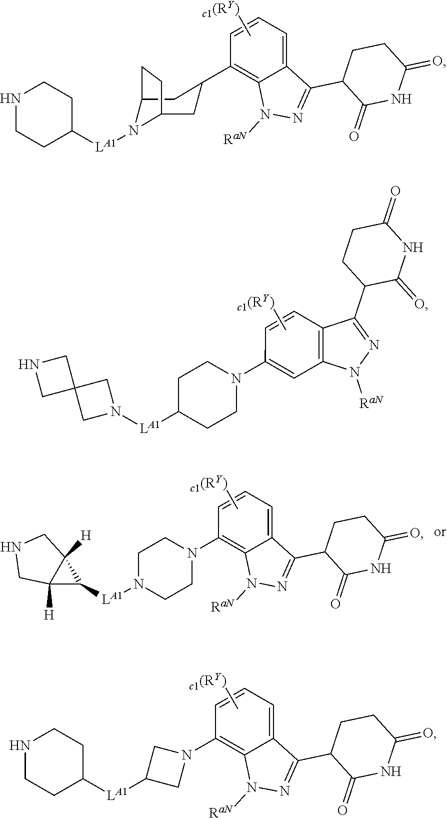

or a salt thereof.

In some embodiments, the compounds of Formula (SI-A) and Formula (SI—B) are compounds of Formula (SI-A-1) and Formula (SI—B-1), respectively:

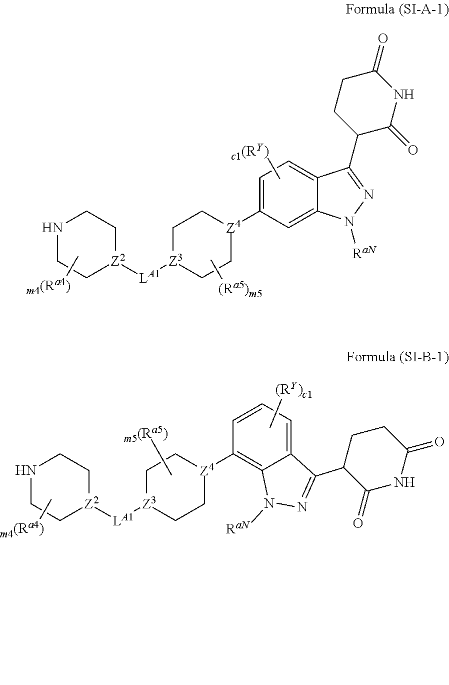

or salts thereof, wherein:
c1 is 0 or 1;
R^{Y} is selected from the group consisting of halo (e.g., —F) and C₁₋₃ alkyl optionally substituted with 1-3 F;
R^{aN} is C₁₋₃ alkyl;
L^{A1} is CH₂, CHMe, or CMe₂;
Z², Z³, and Z⁴ are independently selected from the group consisting of: CH, CR^{a4}, and N, provided that at least one of Z³ and Z⁴ is N; when Z² is N, then Z³ is CH or CR^{a4}; and when Z³ is N, then Z² is CH or CR^{a4};
m4 and m5 are independently 0, 1, or 2; and
each R^{a4} and R^{a5} is independently selected from the group consisting of: —F, CN, C₁₋₃ alkoxy, OH, and C₁₋₃ alkyl optionally substituted with 1-3 F.

In some embodiments of Formula (SI-A-1) and Formula (SI—B-1), Z² is N; Z³ is CH or CR^{a4}; and Z⁴ is N.

In some embodiments of Formula (SI-A-1) and Formula (SI—B-1), Z³ is N; Z² is CH or CR^{a4}; and Z⁴ is N.

In some embodiments of Formula (SI-A-1) and Formula (SI—B-1), Z³ is N; Z² is CH or CR^{a4}; and Z⁴ is CH or CR^{a4}.

In some embodiments of Formula (SI-A-1) and Formula (SI—B-1), one of m4 and m5 is 1; and the other of m4 and m5 is 0. For example, m4 can be 1; and m5 can be 0.

In some embodiments, compounds of Formula (SI-A-1) and Formula (SI—B-1) are selected from the group consisting of:

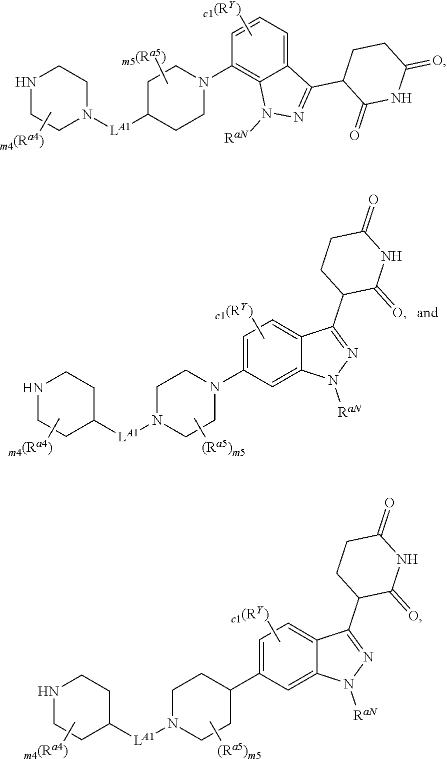

or a salt thereof.

In some embodiments of Formula (SI-A-1) and Formula (SI—B-1), one of m4 and m5 is 1; and the other of m4 and m5 is 0. For example, m4 can be 1; and m5 can be 0.

For example, compounds of Formula (SI-A-1) and Formula (SI—B-1) can be

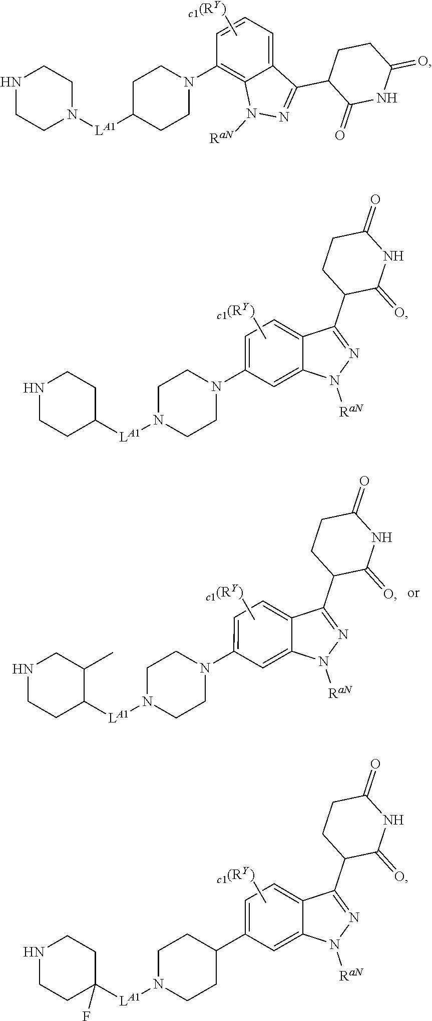

or a salt thereof.

In some embodiments, the compounds of Formula (SI-A) and Formula (SI—B) are compounds of Formula (SI-A-2) and Formula (SI—B-2), respectively:

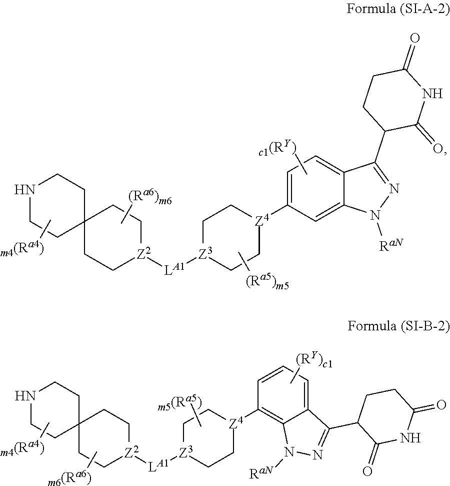

or salts thereof, wherein:
c1 is 0 or 1;
R^{Y} is selected from the group consisting of halo (e.g., —F) and C₁₋₃ alkyl optionally substituted with 1-3 F;
R^{aN} is C₁₋₃ alkyl;
L^{A1} is CH₂, CHMe, or CMe₂;
Z², Z³, and Z⁴ are independently selected from the group consisting of: CH, CR^{a4}, and N, provided that at least one of Z³ and Z⁴ is N; when Z² is N, then Z³ is CH or CR^{a4}; and when Z³ is N, then Z² is CH or CR^{a4};
m4, m5, and m6 are independently 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

In some embodiments of Formula (SI-A-2) and Formula (SI—B-2), Z² is N; Z³ is CH or CR^{a4}; and Z⁴ is N.

In some embodiments of Formula (SI-A-2) and Formula (SI—B-2), Z³ is N; Z² is CH or CR^{a4}; and Z⁴ is N.

For example, compounds of Formula (SI-A-2) and Formula (SI—B-2) can be

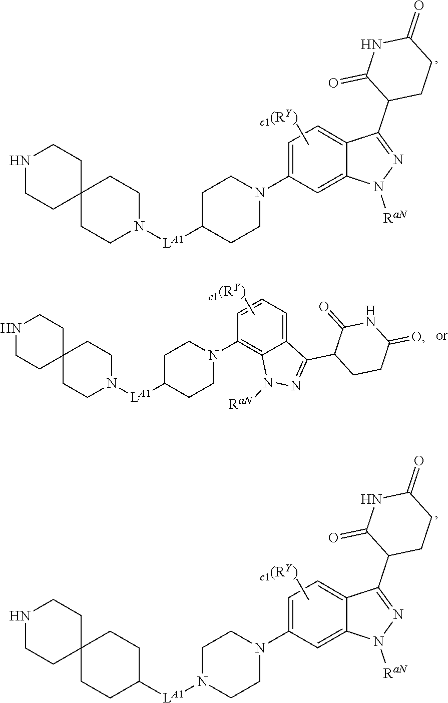

or a salt thereof.

Exemplary embodiments of compounds of one or more of Formulas (SI), (SI-A), (SI—B), (SI-A-1), (SI—B-1), (SI-A-2), and (SI—B-2) include certain compounds described in Examples 2, 3, 4, 5, 6, 7, 8, 9, 10, 11, 12, 19, 22, 24, 28, 29, 30, 31, 32, 33, 34, 37, 38, 39, 41, 42, 45, 46, 51, 53, 57, 58, 59, 60, 62, 64, 71, 79, 83, 85, 86, 90, 95, 107, 108, 114, 115, 116, 117, 118, 123, 124, 125, 126, 130, 131, 133, 134, 135, 136,137, 138, 139, 140, 142, 143, 144, 146, 147, 148, and 151.

Also provided herein are methods of preparing compounds of Formula (I-aa) as depicted in Scheme 1,
the methods comprising reacting compounds of Formula (SII) with compounds of Formula (SI) to provide compounds of Formula (I-aa):

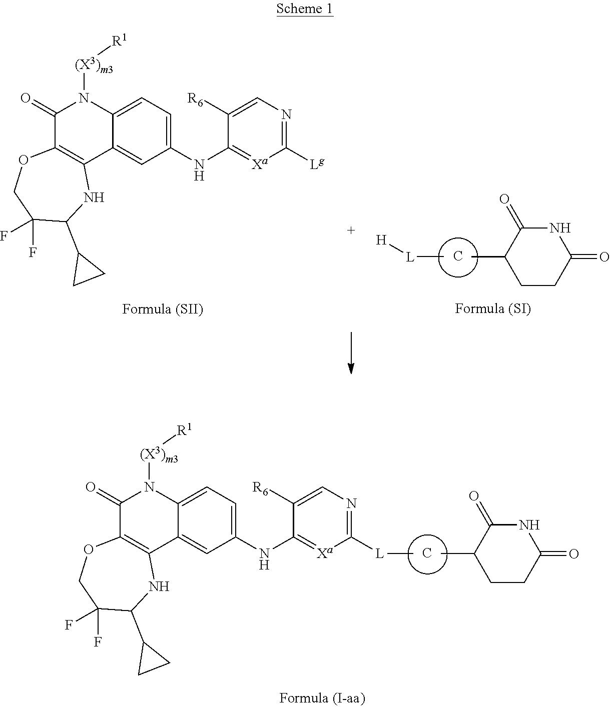

wherein:
L^{g} is halo (e.g., —Cl);
X^{a} is N or CH;
R⁶ is —F or —Cl;
m3 is 1, X³ is C₁₋₃ alkylene, and R¹ is H;
Ring C is selected from the group consisting of:

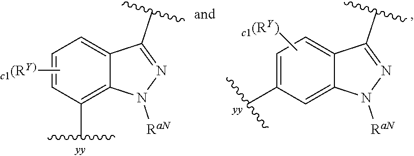

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:
   L^{A4}-L^{A1}-L^{A4}-_{bb};
   L^{A4}-L^{A1}-L^{A1}-L^{A4}-_{bb};
   L^{A4}-L^{A4}-_{bb};
   L^{A4}-C(═O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments, the methods comprise reacting the compounds of Formula (SII) with the compounds of Formula (SI) under conditions suitable for S_{N}Ar reactions. In some embodiments, the methods comprise reacting the compounds of Formula (SII) with the compounds of Formula (SI) in the presence of a base (e.g., triethylamine or diisopropylethylamine) in an appropriate solvent. In some embodiments, the solvent is a polar aprotic solvent (e.g., DMSO or MeCN). In some embodiments, the methods comprise heating the compounds of Formula (SII) with the compounds of Formula (SI) at a temperature of at least 60° C. (e.g., heating the reaction at 60-135° C., 70-120° C., 70-110° C., or 80-110° C.).

In some embodiments of the methods, the compounds of Formula (SI) are compounds of Formula (SI-A), thereby providing a compound of the following formula:

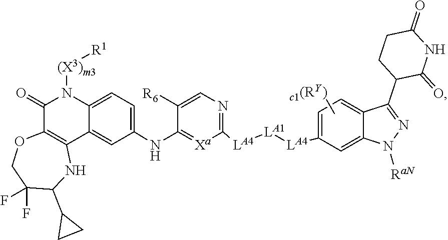

wherein m3, X³, R¹, R⁶, and X^{a} are as defined for Formula (SII) anywhere herein; and
each L^{A4} L^{A1}, c1, R^{Y}, and R^{aN} are as defined for Formula (SI-A) anywhere herein.

In some embodiments of the methods, the compounds of Formula (SI-A) are compounds of Formula (SI-A-1). In some embodiments, the compounds of Formula (SI-A) are compounds of Formula (SI-A-2).

In some embodiments of the methods, the compounds of Formula (SI) are compounds of Formula (SI—B), thereby providing compounds of the following formula:

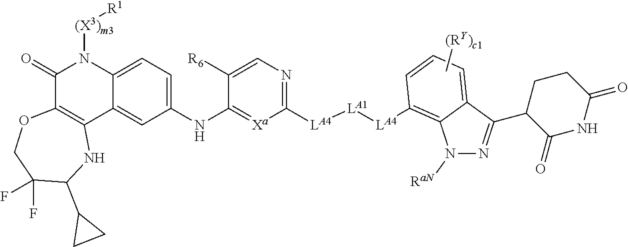

wherein m3, X³, R¹, R⁶, and X^{a} are as defined for Formula (SII) anywhere herein; and
each L^{A4} L^{A1}, c1, R^{Y}, and R^{aN} are as defined for Formula (SI—B) anywhere herein.

In some embodiments, the compounds of Formula (SI—B) are compounds of Formula (SI—B-1). In some embodiments, the compounds of Formula (SI—B) are compounds of Formula (SI—B-2).

### EXAMPLES

In some of the examples disclosed herein, the final product of a described chemical reaction sequence is structurally depicted with enhanced stereochemical notation(s) at one or more stereogenic center(s). Examples of such notations include or1, or2, and the like. In some such examples, in the chemical name of the same compound, each of such stereogenic center(s) is assigned a tentative configuration (e.g., (R)- or (S)-) based on the wedge/dash representation of the structural formula. However, the stereogenic center(s) should be understood to have configurations consistent with the enhanced stereochemical notation(s), as described herein. Accordingly, unless otherwise specified, starting materials and intermediates leading to these compounds incorporate the enhanced stereochemical notations at the corresponding stereogenic centers, notwithstanding the tentative assignments provided in their chemical names.

For example, based on the or1 and or2 notations found on the vicinal stereogenic centers, Compound 465d in Example 112 is a single stereoisomer selected from:
3-(7-(((6S,7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione;
3-(7-(((6S,7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione;
3-(7-(((6R,7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione; and
3-(7-(((6R,7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione.

The intermediate product provided in the first step of the same example incorporates the or] and or2 notations. It is therefore a single stereoisomer selected from:
tert-butyl (6S,7R)-7-((3-(2,6-dioxopiperidin-3-yl)-1-methyl-1H-indazol-7-yl)amino)-6-methyl-2-azaspiro[3.5]nonane-2-carboxylate;
tert-butyl (6S,7S)-7-((3-(2,6-dioxopiperidin-3-yl)-1-methyl-1H-indazol-7-yl)amino)-6-methyl-2-azaspiro[3.5]nonane-2-carboxylate;
tert-butyl (6R,7S)-7-((3-(2,6-dioxopiperidin-3-yl)-1-methyl-1H-indazol-7-yl)amino)-6-methyl-2-azaspiro[3.5]nonane-2-carboxylate; and
tert-butyl (6R,7R)-7-((3-(2,6-dioxopiperidin-3-yl)-1-methyl-1H-indazol-7-yl)amino)-6-methyl-2-azaspiro[3.5]nonane-2-carboxylate.

For further clarification, a chemical name that takes into account the enhanced stereochemical notation(s) is provided for the final product having enhanced stereochemical notation(s) at one or more stereogenic centers. This chemical name is enclosed in brackets (i.e., “[ ]”).

When a stereogenic center is labelled with an asterisk (“*”) in a chemical name of a compound having more than one stereogenic center, the stereogenic center labeled with the asterisk is resolved, but its absolute configuration is either (R)- or (S)-. As such, in a chemical name wherein one stereogenic center is labelled with the asterisk, that stereocenter should be labelled with an or1 enhanced stereochemical notation in its corresponding structure.

For example, the chemical name containing an asterisk for Compound 380b is: [3-(7-(4-((R*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione], as included in Example 9.

When the chemical name of a compound contains two stereogenic centers labelled with asterisks, these two stereogenic centers may have (R) or (S) configurations, either concertedly or independently, in conformity with the orx (e.g., or1 or or2) notations in Table C1 for the corresponding compound.

When two stereogenic centers are labelled or1 and or1 in a compound structure, and they are labelled with asterisks in the chemical name, then the relative stereochemistry between the two stereogenic centers (e.g., syn or anti relationship) is as represented by the name, but the absolute configurations of the two stereogenic centers can vary concertedly (e.g., a compound designated (R*,R*) is one stereoisomer selected from (R,R) and (S,S); for avoidance of doubt, the compound designated (R*,R*) does not have (S,R) or (R,S) configurations across these stereocenters).

When two stereocenters are labelled or1 and or2 in a compound structure, and they are labelled with asterisks in the chemical name, then the configuration of the two stereocenters can vary independently (e.g., a compound designated (R*,R*) is one stereoisomer selected from (R,R), (S,S), (R,S), and (S,R)). For example, the chemical name of a compound having two asterisks for Compound 465d is:

[3-(7-(((6S*,7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione], as included in Example 112.

### General Procedures

### Exemplary Prep-HPLC Conditions

Method A: ACCQ-P rep (Teledyne): Column/dimensions: Gemini NX—C18 column (250×30 mm, 5 μm pore size) 100, Pheinomenex; Mobile phase A: 0.1% FA in Water; Mobile phase B: 0.1% FA in CH₃CN; Gradient: initial 10%, 2 min 10% B, 5.4 min 40% B, 9.6 min 44% B. 11.1 min 100%1B, and 14 min 100% B; Flow Rate: 55 mL/min.
Method B: Column/dimensions: Sunfire C18 (19×300 mm, 7 um; Mobile phase A: 0.1% formic acid in H₂O, Mobile phase B: CH₃CN; Gradient (Time/% B): 0/10, 2/10, 10/25, 16/25, 16.1/100, 20/100, 20/10, 23/10; Flow rate: 18 ml/min.
Method C: Column/Dimensions: X-SELECT-C18 (25×150 mm, 5 um); Mobile phase A: 0.1% Formic acid in H₂O; Mobile phase B: 100% CH₃CN; Gradient (Time/% B): 0/15, 1/15, 10/30, 16/30, 16.1/100, 18/100, 18.1/15, 20/15; Flow rate: 18 ml/min.
Method D: Column/Dimensions: Acquity UPLC BEH C18, 1.7 um (50 mm×2.1 mm); Mobile Phase A: 0.1% Formic Acid in H₂O; B: 0.1% Formic Acid in CH₃CN; Gradient (T % B): 0/3, 1.0/3, 7.0/95, 7.5/95, 9.0/3, 10/3; Flow Rate: 0.5 ml/min.

### Example 1

### Synthesis of 4-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-2-(2,6-dioxopiperidin-3-yl)isoindoline-1,3-dione (Compound 374a)

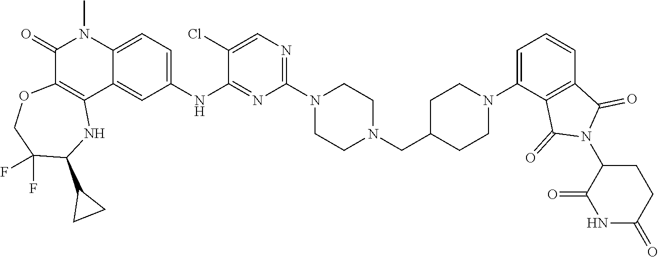 [SMILES: Cn1c(=O)c2c(c3cc(Nc4nc(N5CCN(CC6CCN(c7cccc8c7C(=O)N(C7CCC(=O)NC7=O)C8=O)CC6)CC5)ncc4Cl)ccc31)N[C@@H](C1CC1)C(F)(F)CO2]

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (prepared using published methods, e.g., in International Application Publication Nos. WO 2019/197842 or WO 2021/074620) (67.7 mg, 145 μmol) and 2-(2,6-dioxopiperidin-3-yl)-4-(4-(piperazin-1-ylmethyl)piperidin-1-yl)isoindoline-1,3-dione hydrochloride (prepared using published methods, e.g., in International Application Publication Nos. WO 2021/226262, WO 2020/113233, WO 2021/077010, WO 2019/121562, or WO 2020/264499) (84.5 mg, 178 μmol) in DMSO (1.00 mL) was treated with N,N-diisopropylethylamine (93.4 mg, 126 μL, 723 μmol), warmed to 110° C. for 20 hours, then cooled to room temperature. The reaction mixture was filtered through a 0.45 μm syringe filter and then purified by prep HPLC to afford 4-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-2-(2,6-dioxopiperidin-3-yl)isoindoline-1,3-dione (27.0 mg). 1H NMR (400 MHz, DMSO-d₆) δ 11.00 (s, 1H), 8.77 (s, 1H), 8.11 (d, J=33.6 Hz, 1H), 7.98 (s, 1H), 7.61 (q, J=8.1 Hz, 2H), 7.37 (d, J=9.2 Hz, 1H), 7.25 (t, J=6.9 Hz, 2H), 6.15 (s, 1H), 5.09-4.87 (m, 1H), 4.48-4.20 (m, 2H), 3.62 (d, J=11.7 Hz, 3H), 3.49 (s, 4H), 2.78 (d, J=13.9 Hz, 4H), 2.57 (d, J=24.5 Hz, 2H), 2.28 (d, J=12.6 Hz, 5H), 2.12 (s, 2H), 2.05-1.88 (m, 1H), 1.81-1.61 (m, 3H), 1.27 (d, J=15.2 Hz, 4H), 0.64 (s, 1H), 0.45 (s, 2H), 0.29 (s, 1H). LC-MS (ESI): m/z=871.4 [M+H]⁺.

### Example 2

### Synthesis of 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (Compound 101a)

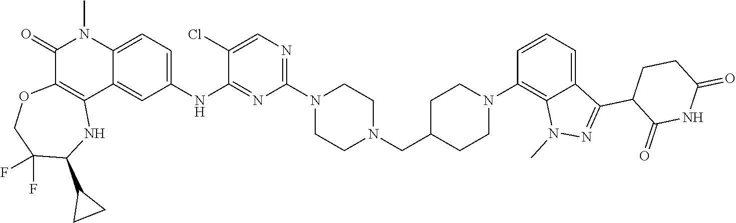 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-((1-((benzyloxy)carbonyl)piperidin-4-yl)methyl)piperazine-1-carboxylate

To a stirred solution of tert-butyl piperazine-1-carboxylate (25.0 g, 0.13 mmol), benzyl-4-formylpiperidine-1-carboxylate (39.60 g, 0.16 mmol), and acetic acid (7.80 g, 0.13 mmol) in DCM (500 mL) was added NaBH(OAc)₃ (42.5 g, 0.20 mmol) at 25° C. and the resulting mixture was stirred at 25° C. for 3 hours. Reaction mixture was quenched with water and extracted with DCM. The organic layers were separated, dried over anhydrous sodium sulfate, filtered, and concentrated. Crude material was purified by column chromatography (SiO₂, 100-200 mesh, 15% ethyl acetate in petroleum ether) to afford tert-butyl 4-((1-((benzyloxy)carbonyl)piperidin-4-yl)methyl)piperazine-1-carboxylate (25.0 g) as a pale-yellow solid. LC-MS (ESI): m/z=418.12 [M+H]⁻.

### Preparation of tert-butyl 4-(piperidin-4-ylmethyl)piperazine-1-carboxylate

To a stirred solution of tert-butyl 4-((1-((benzyloxy)carbonyl)piperidin-4-yl)methyl)piperazine-1-carboxylate (25 g, 0.06 mmol) in MeOH (500 mL) was added Pd/C (7.5 g) and the reaction mixture was stirred under hydrogen atmosphere. The resulting mixture was stirred at room temperature for 16 hours. The reaction mixture was diluted with 30% methanol in DCM, filtered through celite. The filtrate was collected and concentrated to afford the crude product. The crude product was purified by trituration using hexane and diethyl ether to afford tert-butyl 4-(piperidin-4-ylmethyl)piperazine-1-carboxylate (7.0 g) as a yellow solid. 1H NMR (400 MHz, DMSO-d6): δ 3.29 (s, 4H), 3.11-3.08 (m, 2H), 2.66 (t, J=11.60 Hz, 2H), 2.27-2.25 (m, 4H), 2.11 (d, J=6.80 Hz, 2H), 1.74-1.71 (m, 3H), 1.39 (s, 9H), 1.17-1.14 (m, 2H).

### Preparation of 7-bromo-3-iodo-1-methyl-indazole

To a solution of 7-bromo-3-iodo-1H-indazole (20 g, 61.93 mmol, 1.0 equiv.) in THF (50 mL) was added t-BuOK (13.90 g, 123.87 mmol, 2.0 equiv.) at 0° C. for 1 hour. CH₃I (26.37 g, 185.80 mmol, 11.57 mL, 3.0 equiv.) was added to the mixture at 0° C. The mixture was stirred at 20° C. for 2 hours under N₂ atmosphere. The reaction mixture was filtered and concentrated under reduced pressure to obtain a residue. The crude product was purified by reversed-phase HPLC (20-100% 50 min; 100% 20 min, 0.1% formic acid condition) to afford 7-bromo-3-iodo-1-methyl-indazole (15 g) as a yellow solid. LC-MS (ESI): m/z=336.6 [M+H]⁺.

### Preparation of 7-bromo-3-(2,6-dibenzyloxy-3-pyridyl)-1-methyl-indazole

A mixture of 2,6-dibenzyloxy-3-(4,4,5,5-tetramethyl-1,3,2-dioxaborolan-2-yl)pyridine (17.34 g, 41.55 mmol, 1.0 equiv.), 7-bromo-3-iodo-1-methyl-indazole (14 g, 41.55 mmol, 1.0 equiv.), cyclopentyl(diphenyl)phosphane; dichloromethane; dichloropalladium; iron (6.79 g, 8.31 mmol, 0.2 equiv.), and Cs₂CO₃ (40.61 g, 124.65 mmol, 3.0 equiv.) in THF (200 mL) and water (40 mL) was degassed and purged with N2 three times. The mixture was then stirred at 85° C. for 1 hour under N₂ atmosphere. The reaction mixture was concentrated under reduced pressure to remove solvent. The resulting residue was purified by column chromatography (SiO₂, petroleum ether/ethyl acetate=1/0 to 10/1) to afford 7-bromo-3-(2,6-dibenzyloxy-3-pyridyl)-1-methyl-indazole (15.1 g) as a white solid. LC-MS (ESI): m/z=502.3 [M+H]⁺.

### Preparation of tert-butyl 4-((1-(3-(2,6-bis(benzyloxy)pyridin-3-yl)-1-methyl-1H-indazol-7-yl)piperidin-4-yl)methyl)piperazine-1-carboxylate

To a stirred solution of tert-butyl 4-(piperidin-4-ylmethyl)piperazine-1-carboxylate (0.34 g, 1.20 mmol) in 1,4-dioxane (10 mL) was added 7-bromo-3-(2,6-dibenzyloxy-3-pyridyl)-1-methyl-indazole (0.50 g, 1.00 mmol). The reaction was purged with argon for 15 minutes, cesium carbonate (0.98 g, 3.00 mmol) and Pd-PEPPSI-iHeptCl (0.05 g, 0.05 mmol) were added, and the reaction was heated to 120° C. for 3 hours. The reaction mixture was quenched with water and extracted into ethyl acetate. The combined organic layers were washed with a brine solution and dried over anhydrous sodium sulfate, filtered, and concentrated under vacuum to provide a crude product. The crude product was purified by flash column chromatography (SiO₂, 100-200 mesh, 27% ethyl acetate in petroleum ether) to afford tert-butyl 4-((1-(3-(2,6-bis(benzyloxy)pyridin-3-yl)-1-methyl-1H-indazol-7-yl)piperidin-4-yl)methyl)piperazine-1-carboxylate (0.48 g) as a pale yellow solid. LC-MS (ESI): m/z=703.5 [M+H]⁺.

### Preparation of 3-(2,6-bis(benzyloxy)pyridin-3-yl)-1-methyl-7-(4-(piperazin-1-ylmethyl)piperidin-1-yl)-1H-indazole hydrochloride

To a stirred solution of tert-butyl 4-((1-(3-(2,6-bis(benzyloxy)pyridin-3-yl)-1-methyl-1H-indazol-7-yl)piperidin-4-yl)methyl)piperazine-1-carboxylate (0.48 g, 0.68 mmol) in DCM (4.8 mL) was added 4M HCl in dioxane (2.8 mL) at 0° C. The reaction mixture was stirred at room temperature for 16 hours. The reaction mixture was then concentrated under vacuum to provide 3-(2,6-bis(benzyloxy)pyridin-3-yl)-1-methyl-7-(4-(piperazin-1-ylmethyl)piperidin-1-yl)-1H-indazole hydrochloride (0.45 g) as an off-white semi-solid. 1H NMR (400 MHz, DMSO-d₆): δ 7.84 (d, J=8.00 Hz, 1H), 7.48 (d, J=1.60 Hz, 2H), 7.41-7.42 (m, 9H), 7.30-7.31 (m, 1H), 6.57-6.59 (m, 1H), 5.43 (d, J=5.60 Hz, 4H), 4.32 (s, 3H), 3.27-3.25 (m, 2H), 3.08 (s, 4H), 2.54-2.57 (m, 1H), 2.51-2.51 (m, 3H), 2.28-2.30 (m, 1H).

### Preparation of 3-(1-methyl-7-(4-(piperazin-1-ylmethyl)piperidin-1-yl)-1H-indazol-3-yl)piperidine-2,6-dione

To a stirred solution of 3-(2,6-bis(benzyloxy)pyridin-3-yl)-1-methyl-7-(4-(piperazin-1-ylmethyl)piperidin-1-yl)-1H-indazole hydrochloride (0.45 g, 0.75 mmol) in DMF (4.50 mL) was added 10% palladium on carbon (dry) (0.45 g). The reaction was stirred under hydrogen purging with balloon pressure at room temperature for 16 hours. The reaction mixture was diluted with DCM and filtered through celite and washed with excess of 30% THF in DCM. The filtrate was collected and concentrated under vacuum to provide a crude product as a pale yellow solid. The crude product was triturated with methanol and diethyl ether to provide 3-(1-methyl-7-(4-(piperazin-1-ylmethyl)piperidin-1-yl)-1H-indazol-3-yl)piperidine-2,6-dione (0.45 g) as pale yellow solid. LC-MS (ESI): m/z=425 [M+H]⁺.

### Preparation of 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (35.0 mg, 1.00 equiv., 74.7 μmol) and 3-(1-methyl-7-(4-(piperazin-1-ylmethyl)piperidin-1-yl)-1H-indazol-3-yl)piperidine-2,6-dione dihydrochloride (44.6 mg, 1.20 equiv., 89.7 μmol) in MeCN (0.600 mL) was treated with DIPEA (38.6 mg, 52.1 μL, 4.00 equiv., 299 μmol) and warmed to 80° C. for 15 hours. The reaction mixture was cooled to room temperature, filtered, and purified by prep HPLC to afford 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (17.6 mg). ¹H NMR (400 MHz, DMSO) δ 10.87 (s, 1H), 8.84 (s, 1H), 8.18 (d, J=36.1 Hz, 1H), 8.05 (s, 1H), 7.70 (d, J=9.0 Hz, 1H), 7.44 (d, J=9.0 Hz, 1H), 7.36 (d, J=7.2 Hz, 1H), 6.99 (s, 2H), 6.51 (s, 1H), 6.21 (s, 1H), 4.50-4.29 (m, 3H), 4.23 (s, 3H), 3.56 (s, 3H), 3.29 (s, 6H), 2.67 (s, 4H), 2.35 (d, J=17.2 Hz, 5H), 2.22 (s, 5H), 1.85 (s, 1H), 1.70 (s, 1H), 1.35 (s, 2H), 0.70 (s, 1H), 0.52 (s, 2H), 0.36 (s, 1H), 0.07 (s, 2H).

LC-MS (ESI): 2m/z=428.9 [M+H]⁺.

### Example 3. Synthesis of 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (Compound 102a)

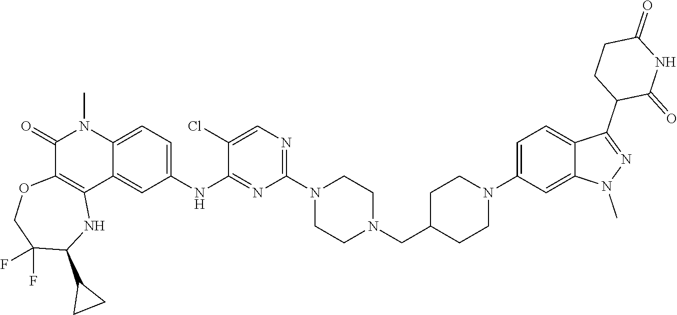 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)CC4)CC3)cc21]

### Preparation of 6-bromo-3-iodo-1-methyl-indazole

To a solution of 6-bromo-3-iodo-1H-indazole (30 g, 92.90 mmol, 1 equiv.) in DMF (200 mL) was added NaH (7.43 g, 185.80 mmol, 60% purity, 2 equiv.) at 0° C. The mixture was stirred for 1 hour. After stirring, Mel (26.37 g, 185.80 mmol, 11.57 mL, 2 equiv.) was added into the solution. The mixture was stirred at 20° C. for 1 hour. After completion, the solution was cooled to 0° C., and saturated NH₄Cl solution (300 mL) was added dropwise. The solution was extracted with ethyl acetate (500 mL×3). The combined organic layers were washed with brine (500 mL), dried over anhydrous sodium sulfate, filtered, and concentrated under reduced pressure to give a residue. The residue was purified by reverse-phase HPLC (0.1% formic acid condition) to afford 6-bromo-3-iodo-1-methyl-indazole (15.3 g) as a brown solid. LC-MS (ESI): m/z=336.8 [M+H]⁺.

### Preparation of 6-bromo-3-(2,6-dibenzyloxy-3-pyridyl)-1-methyl-indazole

A mixture of 6-bromo-3-iodo-1-methyl-indazole (14.8 g, 43.92 mmol, 1 equiv.), 2,6-dibenzyloxy-3-(4,4,5,5-tetramethyl-1,3,2-dioxaborolan-2-yl)pyridine (18.33 g, 43.92 mmol, 1 equiv.), Pd(dppf)Cl₂ (3.21 g, 4.39 mmol, 0.1 equiv.), Cs₂CO₃ (42.93 g, 131.77 mmol, 3 equiv.), and water (10 mL) in THF (100 mL) was degassed and purged with N₂ three times. The mixture was stirred at 60° C. for 16 hours under N2 atmosphere. After completion, the reaction mixture was poured into water (200 mL), and extracted with ethyl acetate (150 mL×3). The combined organic layers were washed with brine (200 mL), dried over anhydrous sodium sulfate, filtered, and concentrated under reduced pressure to give a residue. The residue was purified by column chromatography (SiO₂, petroleum ether/ethyl acetate=100/1 to 20/1) to afford 6-bromo-3-(2,6-dibenzyloxy-3-pyridyl)-1-methyl-indazole (14 g) as a yellow solid. 1H NMR (400 MHz, DMSO-d₆) δ=7.94 (d, J=1.2 Hz, 1H), 7.91 (d, J=8.0 Hz, 1H), 7.61 (d, J=8.4 Hz, 1H), 7.51-7.44 (m, 2H), 7.42-7.23 (m, 8H), 7.10 (dd, J=1.6, 8.8 Hz, 1H), 6.59 (d, J=8.0 Hz, 1H), 5.44 (d, J=8.4 Hz, 4H), 4.04 (s, 3H).

### Preparation of tert-butyl 4-((1-(3-(2,6-bis(benzyloxy)pyridin-3-yl)-1-methyl-1H-indazol-6-yl)piperidin-4-yl)methyl)piperazine-1-carboxylate

To a stirred suspension of 6-bromo-3-(2,6-dibenzyloxy-3-pyridyl)-1-methyl-indazole (1.0 g, 2.0 mmol) in 1,4-dioxane (15 mL) and DMF (15 mL) was added tert-butyl 4-(piperidin-4-ylmethyl)piperazine-1-carboxylate (0.68 g, 2.4 mmol) and cesium carbonate (1.95 g, 6.0 mmol). The reaction was purged with argon for 15 minutes, RuPhos (0.18 g, 0.4 mmol) and RuPhos-Pd-G3 (0.08 g, 0.1 mmol) was added, and the reaction was heated at 100° C. for 3 hours.

The reaction mixture was cooled to room temperature and extracted with ethyl acetate. The combined organic layers were washed with brine solution, dried over anhydrous sodium sulfate, filtered, and concentrated to provide a crude product. The crude product was purified by flash column chromatography (amine silica, 35-60 mesh, 50% ethyl acetate in petroleum ether) to afford tert-butyl 4-((1-(3-(2,6-bis(benzyloxy)pyridin-3-yl)-1-methyl-1H-indazol-6-yl)piperidin-4-yl)methyl)piperazine-1-carboxylate (0.7 g) as a brown semi-solid. LC-MS (ESI): m/z=703.72 [M+H]⁺.

### Preparation of tert-butyl 4-((1-(3-(2,6-dioxopiperidin-3-yl)-1-methyl-1H-indazol-6-yl)piperidin-4-yl)methyl)piperazine-1-carboxylate

To a stirred solution of tert-butyl 4-((1-(3-(2,6-bis(benzyloxy)pyridin-3-yl)-1-methyl-1H-indazol-6-yl)piperidin-4-yl)methyl)piperazine-1-carboxylate (0.70 g, 0.99 mmol) in a mixture of isopropyl alcohol (10 mL), ethyl acetate (10 mL), and DMF (7 mL) was added 10% Pd/C (0.69 g, 6.50 mmol). The reaction was stirred under a hydrogen atmosphere with balloon pressure for 12 hours at room temperature. The reaction mixture was filtered through celite using 50% THF/DCM. The filtrate was concentrated under reduced pressure to afford tert-butyl 4-((1-(3-(2,6-dioxopiperidin-3-yl)-1-methyl-1H-indazol-6-yl)piperidin-4-yl)methyl)piperazine-1-carboxylate (0.40 g) as a brown solid. LC-MS (ESI): m/z=525.55 [M+H]⁺.

### Preparation of 3-(1-methyl-6-(4-(piperazin-1-ylmethyl)piperidin-1-yl)-1H-indazol-3-yl)piperidine-2,6-dione hydrochloride

To a stirred solution of tert-butyl 4-((1-(3-(2,6-dioxopiperidin-3-yl)-1-methyl-1H-indazol-6-yl)piperidin-4-yl)methyl)piperazine-1-carboxylate (0.40 g, 0.76 mmol) in DCM (4 mL) was added 4M HCl in dioxane (2 mL). The reaction was stirred at room temperature for 3 hours. The reaction mixture was concentrated under reduced pressure to afford 3-(1-methyl-6-(4-(piperazin-1-ylmethyl)piperidin-1-yl)-1H-indazol-3-yl)piperidine-2,6-dione hydrochloride (0.40 g) as a brown solid. LC-MS (ESI): m/z=425.40 [M+H]⁺.

### Preparation of 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (72.0 mg, 1.00 equiv., 154 μmol) and 3-(1-methyl-6-(4-(piperazin-1-ylmethyl)piperidin-1-yl)-1H-indazol-3-yl)piperidine-2,6-dione hydrochloride (84.9 mg, 1.20 equiv., 184 μmol) in MeCN (1.00 mL) was treated with DIEA (79.5 mg, 107 μL, 4.00 equiv., 615 μmol) and warmed to 80° C. for 19 hours, then cooled to room temperature. The reaction mixture was diluted with DMSO, concentrated under reduced pressure, filtered, and purified by prep HPLC to afford 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (70 mg). 1H NMR (400 MHz, DMSO) δ 10.83 (s, 1H), 8.84 (s, 1H), 8.22 (s, 1H), 8.05 (s, 1H), 7.70 (d, J=9.1 Hz, 1H), 7.45 (dd, J=14.6, 9.1 Hz, 2H), 6.90 (d, J=8.7 Hz, 1H), 6.81 (s, 1H), 6.21 (s, 1H), 4.40 (m, 2H), 4.24 (m, 1H), 3.88 (s, 3H), 3.77 (d, J=11.6 Hz, 2H), 3.64-3.51 (m, 4H), 2.73 (d, J=12.5 Hz, 1H), 2.67 (s, 2H), 2.60 m, 1H), 2.33 (m, 5H), 2.17 (d, J=6.6 Hz, 3H), 1.80 (d, J=12.5 Hz, 2H), 1.71 (m, 2H), 1.25 (m, J=12.1 Hz, 4H), 0.71 (m, 1H), 0.59-0.46 (m, 3H), 0.36 (m, 1H). LC-MS (ESI): m/z=468.2 [M+H]⁺.

### Example 4. Synthesis of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-1-methyl-1H-indazol-3-yl)piperidine-2,6-dione (Compound 130a)

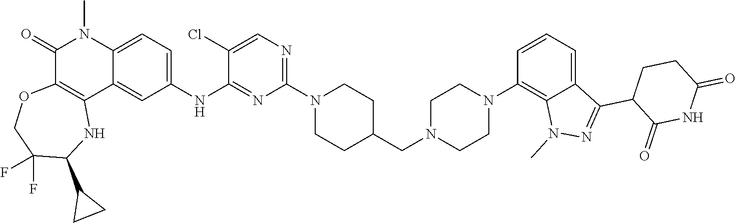 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)[C@H](C7CC7)N6)n5)CC4)CC3)c21]

### Preparation of benzyl 4-((1-(tert-butoxycarbonyl)piperidin-4-yl)methyl)piperazine-1-carboxylate

To a solution of benzyl piperazine-1-carboxylate (10.0 g, 45.39 mmol), tert-butyl 4-formylpiperidine-1-carboxylate (11.61 g, 54.47 mmol), and NaBH(OAc)₃ (9.62 g, 45.39 mmol) in DCM (200 mL) was added acetic acid (2.72 g, 45.39 mmol). The reaction was stirred at room temperature for 16 h. The reaction mixture was quenched with ice-cold water (500 mL) and extracted into DCM (3×250 mL). The combined organic layers were washed with brine solution (500 mL) and dried over anhydrous sodium sulfate, filtered, and dried under vacuum to get crude product. The crude product was purified by column chromatography (SiO₂; 40% ethyl acetate in petroleum ether) to afford benzyl 4-((1-(tert-butoxycarbonyl)piperidin-4-yl)methyl)piperazine-1-carboxylate (6.0 g) as colorless liquid. LC-MS (ESI): m/z=418.31 [M+H]⁺.

### Preparation of tert-butyl 4-(piperazin-1-ylmethyl)piperidine-1-carboxylate

To a stirred solution of benzyl 4-((1-(tert-butoxycarbonyl)piperidin-4-yl)methyl)piperazine-1-carboxylate (6.0 g, 14.36 mmol) in THF (180 mL) was added 20% Pd(OH)₂ on carbon (3.0 g). The reaction was put under a hydrogen atmosphere (60 psi) at room temperature and stirred for 16 hours. Reaction mixture was filtered through celite and concentrated to obtain the crude product. The crude product was washed with n-pentane and dried to afford tert-butyl 4-(piperazin-1-ylmethyl)piperidine-1-carboxylate (3.0 g) as a colorless liquid. LC-MS (ESI): m/z=284.16 [M+H]⁺.

### Preparation of tert-butyl 4-((4-(3-(2,6-bis(benzyloxy)pyridin-3-yl)-1-methyl-1H-indazol-7-yl)piperazin-1-yl)methyl)piperidine-1-carboxylate

A solution of 3-(2,6-bis(benzyloxy)pyridin-3-yl)-7-bromo-1-methyl-1H-indazole (1.0 g, 2.0 mmol), tert-butyl 4-(piperazin-1-ylmethyl)piperidine-1-carboxylate (0.56 g, 2.0 mmol), and cesium carbonate (1.95 g, 6.0 mmol) in 1,4-dioxane (20 mL) was degassed with argon for 5 minutes. Pd-PEPPSI-iHeptCl (0.97 g, 0.1 mmol) was added to the reaction and the reaction was again degassed for 5 minutes. The reaction mixture was then heated at 80° C. for 16 hours. The reaction mixture was quenched with ice-cold water (50 mL) and extracted into ethyl acetate (3×100 mL). The combined organic layers were washed with brine solution (50 mL), dried over anhydrous sodium sulfate, filtered, and dried under vacuum to provide a crude product. The crude product was purified by column chromatography (SiO₂; 40% ethyl acetate in petroleum ether) to afford tert-butyl 4-((4-(3-(2,6-bis(benzyloxy)pyridin-3-yl)-1-methyl-1H-indazol-7-yl)piperazin-1-yl)methyl)piperidine-1-carboxylate (1.0 g) as a pale brown semi solid. LC-MS (ESI): m/z=703.80 [M+H]⁺.

### Preparation of tert-butyl 4-((4-(3-(2,6-dioxopiperidin-3-yl)-1-methyl-1H-indazol-7-yl)piperazin-1-yl)methyl)piperidine-1-carboxylate

To a stirred solution of tert-butyl 4-((4-(3-(2,6-bis(benzyloxy)pyridin-3-yl)-1-methyl-1H-indazol-7-yl)piperazin-1-yl)methyl)piperidine-1-carboxylate (1.0 g, 1.42 mmol) in THF (60 mL) was added 20% Pd(OH)₂ on carbon (1.0 g). The reaction was put under hydrogen atmosphere (60 psi) and stirred at room temperature for 6 hours. The reaction mixture was filtered through celite and concentrated to obtain a crude product. The crude product was washed with n-pentane and dried to afford tert-butyl 4-((4-(3-(2,6-dioxopiperidin-3-yl)-1-methyl-1H-indazol-7-yl)piperazin-1-yl)methyl)piperidine-1-carboxylate (0.70 g) as a brown solid. LC-MS (ESI): m/z=525.61 [M+H]⁺.

### Preparation of 3-(1-methyl-7-(4-(piperidin-4-ylmethyl)piperazin-1-yl)-1H-indazol-3-yl)piperidine-2,6-dione

A stirred solution of tert-butyl 4-((4-(3-(2,6-dioxopiperidin-3-yl)-1-methyl-1H-indazol-7-yl)piperazin-1-yl)methyl)piperidine-1-carboxylate (0.70 g, 1.33 mmol) in DCM (14 mL) was cooled to 0° C. and TFA (7.0 mL) was added. The reaction was allowed to warm to room temperature over 3 hours. The reaction mixture was then concentrated to obtain crude product. The crude product was washed with n-pentane to obtain 3-(1-methyl-7-(4-(piperidin-4-ylmethyl)piperazin-1-yl)-1H-indazol-3-yl)piperidine-2,6-dione (0.50 g) as a brown gummy solid.

LC-MS (ESI): m/z=425.51 [M+H]⁺.

### Preparation of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-1-methyl-1H-indazol-3-yl)piperidine-2,6-dione

To a stirred solution of 3-(1-methyl-7-(4-(piperidin-4-ylmethyl)piperazin-1-yl)-1H-indazol-3-yl)piperidine-2,6-dione (0.15 g, 0.35 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.13 g, 0.28 mmol) in DMSO was added N,N-diisopropylethylamine (0.36 g, 2.82 mmol). The reaction mixture was stirred at 100° C. for 16 hours. The reaction mixture was poured into ice cold water and stirred for 15 minutes. The resulting precipitate was filtered and dried to get crude product. The crude product was purified by prep HPLC to afford 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-1-methyl-1H-indazol-3-yl)piperidine-2,6-dione (0.050 g) as an off white solid. 1H NMR (400 MHz, DMSO-d₆): δ 10.92 (s, 1H), 8.77 (s, 1H), 8.18 (d, J=2.00 Hz, 1H), 8.03 s, 1H), 7.76-7.73 (m, 1H), 7.45 (d, J=9.20 Hz, 1H), 7.39 (t, J=4.80 Hz, 1H), 7.03-7.02 (m, 1H), 6.17 (br s, 1H), 4.44-4.32 (m, 5H), 4.25 (s, 3H), 3.57 (s, 3H), 3.20-2.75 (m, 11H), 2.67-2.60 (m, 2H), 2.33-2.17 (m, 4H), 1.75-1.72 (m, 3H), 1.04-1.02 (m, 1H), 0.53-0.35 (m, 4H). LC-MS (ESI): m/z=856.46 [M+H]⁺.

### Example 5. Synthesis of 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione (Compound 214a)

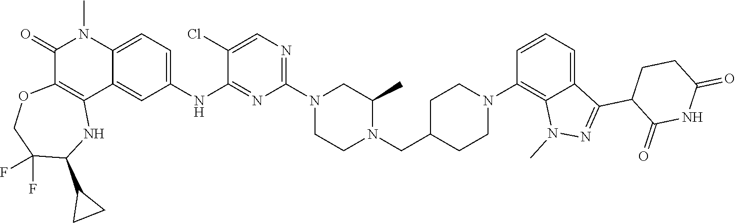 [SMILES: C[C@@H]1CN(c2ncc(Cl)c(Nc3ccc4c(c3)c3c(c(=O)n4C)OCC(F)(F)[C@H](C4CC4)N3)n2)CCN1CC1CCN(c2cccc3c(C4CCC(=O)NC4=O)nn(C)c23)CC1]

### Preparation of tert-butyl (R)-4-((1-((benzyloxy)carbonyl)piperidin-4-yl)methyl)-3-methylpiperazine-1-carboxylate

To a solution of benzyl piperazine-1-carboxylate (8.8 g, 36.0 mmol), tert-butyl 4-formylpiperidine-1-carboxylate (6.0 g, 30.0 mmol) and NaBH(OAc)₃ (9.4 g, 45.0 mmol) in DCM (120 mL) was added acetic acid (1.8 mL, 30.0 mmol). The reaction was stirred at room temperature for 10 hours. The reaction mixture was quenched with cold water (200 mL) and extracted into DCM (3×200 mL). The combined organic layers were washed with brine solution (200 mL), dried over anhydrous sodium sulfate, filtered, and dried under vacuum to provide a crude product. The crude product was purified by column chromatography (SiO₂; 40% ethyl acetate in petroleum ether) to afford tert-butyl (R)-4-((1-((benzyloxy)carbonyl)piperidin-4-yl)methyl)-3-methylpiperazine-1-carboxylate (6.1 g) as a colorless liquid. LC-MS (ESI): m/z=432.35 [M+H]⁺.

### Preparation of tert-butyl (R)-3-methyl-4-(piperidin-4-ylmethyl)piperazine-1-carboxylate

To a stirred solution of tert-butyl (R)-4-((1-((benzyloxy)carbonyl)piperidin-4-yl)methyl)-3-methylpiperazine-1-carboxylate (6.0 g, 13.9 mmol) in THF (180 mL) was added 20% Pd(OH)₂ (3.0 g, 50%, w/w). The reaction was put under hydrogen atmosphere (60 psi) and stirred at room temperature for 16 hours. The reaction mixture was filtered through celite and concentrated to obtain a crude product. The crude product was washed with n-pentane and dried to afford tert-butyl (R)-3-methyl-4-(piperidin-4-ylmethyl)piperazine-1-carboxylate (3.8 g) as a colorless liquid.

LC-MS (ESI): m/z=298.24 [M+H]⁺.

### Preparation of tert-butyl (R)-4-((1-(3-(2,6-bis(benzyloxy)pyridin-3-yl)-1-methyl-1H-indazol-7-yl)piperidin-4-yl)methyl)-3-methylpiperazine-1-carboxylate

To a stirred solution of 3-(2,6-bis(benzyloxy)pyridin-3-yl)-7-bromo-1-methyl-1H-indazole (1.2 g, 2.40 mmol) and tert-butyl (R)-3-methyl-4-(piperidin-4-ylmethyl)piperazine-1-carboxylate (0.85 g, 2.88 mmol) in 1,4-dioxane (24 mL) was added cesium carbonate (2.34 g, 7.20 mmol). The reaction was degassed with argon for 10 minutes, Pd-PEPPSI-iHeptCl (0.11 g, 0.12 mmol) was added, and the reaction was heated at 100° C. for 12 hours. The reaction mixture was filtered through celite and concentrated to obtain a crude product. The crude product was purified by flash column chromatography by using (230-400 mesh silica gel, 30% ethyl acetate in petroleum ether) to afford tert-butyl (R)-4-((1-(3-(2,6-bis(benzyloxy)pyridin-3-yl)-1-methyl-1H-indazol-7-yl)piperidin-4-yl)methyl)-3-methylpiperazine-1-carboxylate (1.0 g) as a pale yellow semi-solid.

LC-MS (ESI): m/z=717.64 [M+H]⁺.

### Preparation of tert-butyl (3R)-4-((1-(3-(2,6-dioxopiperidin-3-yl)-1-methyl-1H-indazol-7-yl)piperidin-4-yl)methyl)-3-methylpiperazine-1-carboxylate

To a stirred solution of tert-butyl (R)-4-((1-(3-(2,6-bis(benzyloxy)pyridin-3-yl)-1-methyl-1H-indazol-7-yl)piperidin-4-yl)methyl)-3-methylpiperazine-1-carboxylate (1.0 g, 1.39 mmol) in THF (20 mL) was added 20% Pd(OH)₂ (100% w/w, 1.0 g). The reaction was put under hydrogen atmosphere (60 psi) and stirred at room temperature for 6 hours. The reaction mixture was filtered through celite and concentrated to obtain tert-butyl (3R)-4-((1-(3-(2,6-dioxopiperidin-3-yl)-1-methyl-1H-indazol-7-yl)piperidin-4-yl)methyl)-3-methylpiperazine-1-carboxylate (0.75 g) as a pale brown semi-solid. LC-MS (ESI): m/z=539.37 [M+H]⁺.

### Preparation of 3-(1-methyl-7-(4-(((R)-2-methylpiperazin-1-yl)methyl)piperidin-1-yl)-1H-indazol-3-yl)piperidine-2,6-dione

To a stirred solution of tert-butyl (3R)-4-((1-(3-(2,6-dioxopiperidin-3-yl)-1-methyl-1H-indazol-7-yl)piperidin-4-yl)methyl)-3-methylpiperazine-1-carboxylate (0.70 g, 1.3 mmol) in DCM (14 mL) was added TFA (2.1 mL). The reaction was stirred at room temperature for 3 hours. The reaction mixture was concentrated and co-distilled with petroleum ether to obtain 3-(1-methyl-7-(4-(((R)-2-methylpiperazin-1-yl)methyl)piperidin-1-yl)-1H-indazol-3-yl)piperidine-2,6-dione (0.60 g) as a brown solid. LC-MS (ESI): m/z=439.57 [M+H]⁺.

### Preparation of 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione

To a stirred solution of 3-(1-methyl-7-(4-(((R)-2-methylpiperazin-1-yl)methyl)piperidin-1-yl)-1H-indazol-3-yl)piperidine-2,6-dione (0.30 g, 0.68 mmol) in DMSO (6 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.12 g, 0.27 mmol) and DIPEA (1.1 mL, 6.8 mmol).

The reaction was stirred at 100° C. for 12 hours. The reaction mixture was concentrated under vacuum to obtain a crude product. The crude product was purified by prep HPLC to afford 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione (74 mg) as an off-white solid. ¹H NMR 400 MHz, DMSO-d₆: δ 10.87 (s, 1H), 8.84 (s, 1H), 8.20 (s, 1H), 8.14 (s, 2H), 8.04 (s, 1H), 7.70 (dd, J=1.60, 9.00 Hz, 1H), 7.44 (d, J=9.20 Hz, 1H), 7.36 (dd, J=2.00, 6.80 Hz, 1H), 7.03-6.98 (m, 2H), 6.22 (s, 1H), 4.31-4.32 (m, 3H), 4.24 (s, 3H), 4.00 (s, 2H), 3.56 (s, 3H), 3.32 (br, 3H), 2.84-2.82 (m, 2H), 2.61-2.59 (m, 4H), 2.31-2.30 (m, 2H), 2.02-1.99 (m, 4H), 1.72 (br s, 1H), 1.62 (br s, 1H), 1.33-1.31 (m, 2H), 0.97 (d, J=6.00 Hz, 3H), 0.71-.69 (m, 1H), 0.53-0.50 (m, 2H), 0.36-0.35 (m, 1H). LC-MS (ESI): m/z=868.44 [M−H]⁻.

### Example 6. 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-1-methyl-1H-indazol-3-yl)piperidine-2,6-dione (Compound 400a)

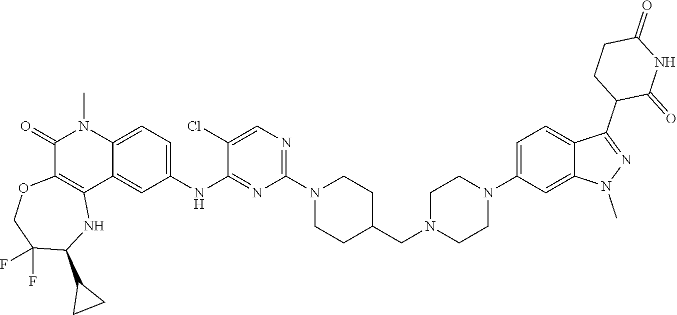 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-((4-(3-(2,6-bis(benzyloxy)pyridin-3-yl)-1-methyl-1H-indazol-6-yl)piperazin-1-yl)methyl)piperidine-1-carboxylate

A solution of 3-(2,6-bis(benzyloxy)pyridin-3-yl)-6-bromo-1-methyl-1H-indazole (0.8 g, 1.59 mmol), tert-butyl 4-(piperazin-1-ylmethyl)piperidine-1-carboxylate (0.9 g, 2.0 mmol), and cesium carbonate (1.55 g, 4.77 mmol) in 1,4-dioxane (16 mL) was degassed with argon for 5 minutes. RuPhos-Pd-G3 (0.066 g, 0.079 mmol) and RuPhos (0.069 g, 0.15 mmol) was added. The reaction was degassed for 5 minutes and then heated at 100° C. for 16 hours. The reaction mixture was quenched with ice-cold water (40 mL) and extracted into ethyl acetate (3×100 mL). The combined organic layers were washed with brine solution (40 mL), dried over anhydrous sodium sulfate, filtered, and dried under vacuum to provide a crude product. The crude product was purified by column chromatography (SiO₂; 50% ethyl acetate in petroleum ether) to afford tert-butyl 4-((4-(3-(2,6-bis(benzyloxy)pyridin-3-yl)-1-methyl-1H-indazol-6-yl)piperazin-1-yl)methyl)piperidine-1-carboxylate (0.8 g) as a pale brown semi-solid. LC-MS (ESI): m/z=703.80. [M+H]⁺.

### Preparation of tert-butyl 4-((4-(3-(2,6-dioxopiperidin-3-yl)-1-methyl-1H-indazol-6-yl)piperazin-1-yl)methyl)piperidine-1-carboxylate

To a stirred solution of tert-butyl 4-((4-(3-(2,6-bis(benzyloxy)pyridin-3-yl)-1-methyl-1H-indazol-6-yl)piperazin-1-yl)methyl)piperidine-1-carboxylate (1.0 g, 1.13 mmol) in THF (40 mL) was added 20% Pd(OH)₂ on carbon (0.8 g). The reaction was stirred under hydrogen atmosphere (60 psi) at room temperature for 6 hours. The reaction mixture was filtered through celite, the celite was washed with THF, and the filtrate was concentrated. The crude product was washed with n-pentane and dried to afford tert-butyl 4-((4-(3-(2,6-dioxopiperidin-3-yl)-1-methyl-1H-indazol-6-yl)piperazin-1-yl)methyl)piperidine-1-carboxylate (0.5 g) as a brown solid. LC-MS (ESI): m/z=525.66 [M+H]⁺.

### Preparation of 3-(1-methyl-6-(4-(piperidin-4-ylmethiyl)piperazin-1-yl)-1H-indazol-3-yl)piperidine-2,6-dione

A stirred solution of tert-butyl 4-((4-(3-(2,6-dioxopiperidin-3-yl)-1-methyl-1H-indazol-6-yl)piperazin-1-yl)methyl)piperidine-1-carboxylate (0.5 g, 0.95 mmol) in DCM (10 mL) was cooled to 0° C. and TFA was added (5.0 mL). The reaction mixture was stirred at room temperature for 3 hours. The reaction mixture was concentrated to obtain a crude product. The crude product was washed with n-pentane to afford 3-(1-methyl-6-(4-(piperidin-4-yl)methyl)piperazin-1-yl)-1H-indazol-3-yl)piperidine-2,6-dione (0.350 g) as a brown gummy solid. LC-MS (ESI): m/z=425.52 [M+H]⁺.

### Preparation of 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-1-methyl-1H-indazol-3-yl)piperidine-2,6-dione

To a stirred solution 3-(1-methyl-6-(4-(piperidin-4-ylmethyl)piperazin-1-yl)-1H-indazol-3-yl)piperidine-2,6-dione (0.150 g, 0.35 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.13 g, 0.28 mmol) in DMSO was added N,N-diisopropylethylamine (0.36 g, 2.82 mmol). The reaction mixture was stirred at 100° C. for 16 hours. The reaction mixture was poured into ice cold water and stirred for 15 minutes. The resulting precipitate was filtered and dried to obtain a crude product. The crude product was purified by prep HPLC to afford 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-1-methyl-1H-indazol-3-yl)piperidine-2,6-dione (0.035) as a brown solid. ¹H NMR (400 MHz, DMSO-d₆): δ 10.90 (s, 1H), 8.77 (s, 1H), 8.18 (d, J=1.60 Hz, 1H), 8.03 (s, 1H), 7.74 (dd, J=1.60, 9.00 Hz, 1H), 7.50-7.43 (m, 2H), 6.91 (d, J=8.80 Hz, 1H), 6.83 (s, 1H), 6.17 (s, 1H), 4.45-4.36 (m, 4H), 4.27-4.23 (m, 1H), 3.89 (s, 3H), 3.56 (s, 3H), 3.33-3.31 (br, 5H), 2.91-2.79 (m, 2H), 2.67-2.56 (m, 2H), 2.50-0.2.49 (m, 4H), 2.32-2.28 (m, 1H), 2.18-2.15 (m, 3H), 1.75-1.71 (m, 3H), 1.35-1.33 (m, 1H), 1.03-1.01 (m, 2H), 0.72-0.70 (m, 1H), 0.53-0.50 (m, 2H), 0.36-0.34 (m, 1H). LC-MS (ESI): m/z=856.46 [M+H]⁺.

### Example 7. 3-(7-(4-((S)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(7-(4-((S*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione](Compound 380a)

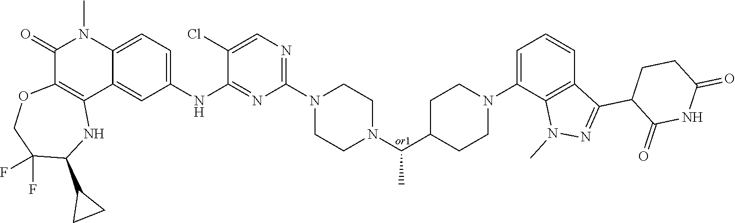 [SMILES: C[C@@H](C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)[C@H](C4CC4)N3)n2)CC1]

### Preparation of benzyl 4-acetylpiperidine-1-carboxylate

A stirred solution of 1-(piperidin-4-yl)ethan-1-one hydrochloride (10.0 g, 61.33 mmol) in DCM (200 mL) was cooled to 0° C. under N2 atmosphere. TEA (25.64 mL, 184 mmol) was added, and the reaction was stirred for 5 minutes. Benzyl chloroformate (13.1 mL, 91.99 mmol) was added and the resultant reaction mixture was allowed to warm to room temperature over 16 hours while stirring. The reaction mixture was quenched with cold water and extracted in DCM. The combined organic layers were dried over anhydrous sodium sulfate, filtered, and concentrated to provide a crude product. The crude product was purified by flash column chromatography (SiO₂, 100-200, 25% ethyl acetate in petroleum ether) to afford benzyl 4-acetylpiperidine-1-carboxylate as a white solid (13.0 g). LC-MS (ESI): m/z=284.31 [M+Na]⁺.

### Preparation of tert-butyl 4-(1-(1-((benzyloxy)carbonyl)piperidin-4-yl)ethyl)piperazine-1-carboxylate

A stirred solution of benzyl 4-acetylpiperidine-1-carboxylate (10 g, 38.26 mmol) and tert-butyl piperazine-1-carboxylate (14.25 g, 76.53 mmol) in ethanol (200 mL) was cooled to 0° C. under N2 atmosphere. Titanium(IV) isopropoxide (10 mL) was added, and the reaction was allowed to warm to room temperature over 2 hours under N2 atmosphere. Sodium cyanoborohydride (7.21 g, 114.80 mmol) was added, and the reaction mixture was heated to 50° C. for 16 hours. The reaction mixture was quenched with cold water and filtered through celite and the celite was washed with ethyl acetate. The combined organic layers were washed with brine, dried over anhydrous sodium sulfate, filtered, and concentrated to provide a crude product. The crude product was purified by flash column chromatography (SiO₂, 100-200, 10% ethyl acetate in petroleum ether) to afford tert-butyl 4-(1-(1-((benzyloxy)carbonyl)piperidin-4-yl)ethyl)piperazine-1-carboxylate as a white solid (13.0 g). LC-MS (ESI): m/z=432.50 [M+H]⁺.

### Preparation of tert-butyl 4-(1-(1-((benzyloxy)carbonyl)piperidin-4-yl)ethyl) piperazine-1-carboxylate (Isomers 1 and 2)

The tert-butyl 4-(1-(1-((benzyloxy)carbonyl)piperidin-4-yl)ethyl)piperazine-1-carboxylate enantiomers were separated via chiral HPLC SFC purification to afford tert-butyl 4-(1-(1-((benzyloxy)carbonyl)piperidin-4-yl)ethyl)piperazine-1-carboxylate (Isomer 1) (3.6 g) and tert-butyl 4-(1-(1-((benzyloxy)carbonyl)piperidin-4-yl)ethyl)piperazine-1-carboxylate (Isomer 2) (3.5 g).

### Preparation of tert-butyl 4-(1-(piperidin-4-yl)ethyl)piperazine-1-carboxylate (Isomer 1)

A stirred solution of tert-butyl 4-(1-(1-((benzyloxy)carbonyl)piperidin-4-yl)ethyl)piperazine-1-carboxylate (Isomer 1) (1.5 g, 3.47 mmol) in THF (30 mL) was degassed with nitrogen for 5 minutes. 10% Pd/C (0.37 g, 0.37 mmol) was added and the reaction was put under hydrogen atmosphere (50 psi) and stirred at room temperature for 16 hours. The reaction mixture was diluted with THF (100 mL) and filtered through celite. The filtrate was collected and concentrated to afford tert-butyl 4-(1-(piperidin-4-yl)ethyl)piperazine-1-carboxylate (Isomer 1) as a brown semi-solid (1.0 g). LC-MS (ESI): m/z=298.24.

### Preparation of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 1)

A stirred solution of 3-(2,6-bis(benzyloxy)pyridin-3-yl)-7-bromo-1-methyl-1H-indazole (1.4 g, 2.8 mmol) and tert-butyl 4-(1-(piperidin-4-yl)ethyl)piperazine-1-carboxylate (Isomer 1) (1.0 g, 3.3 mmol) in 1,4-dioxane (28 mL) was put under N₂ atmosphere. Cesium carbonate (1.82 g, 5.6 mmol) was added and the reaction was degassed with N2 for 10 minutes. PEPPSI-iHeptCl (0.13 g, 0.14 mmol) was added and the reaction was stirred at 100° C. for 3 hours. The reaction mixture was filtered through celite and the celite was washed with ethyl acetate. The combined organic layers were concentrated and purified by flash column chromatography (SiO₂, 100-200, 27% ethyl acetate in petroleum ether) to afford tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 1) (2.0 g). LC-MS (ESI): m/z=717.25 [M+H]⁺.

### Preparation of tert-butyl 4-(1-(1-(3-(2,6-dioxopiperidin-3-yl)-1-methyl-1H-indazol-7-yl)piperidin-4-yl)ethyl)piperazine-1-carboxylate (Isomer 1)

A stirred solution of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 1) (1.0 g, 1.39 mmol) in THF (50 mL) was degassed with nitrogen for 5 minutes. 20% Pd(OH)₂/C (1.0 g, 1.39 mmol) was added and the reaction was put under hydrogen atmosphere with balloon pressure at room temperature for 16 hours. The reaction mixture was diluted with ethyl acetate (350 mL) and filtered through celite.

The filtrate was collected and concentrated to afford tert-butyl 4-(1-(1-(3-(2,6-dioxopiperidin-3-yl)-1-methyl-1H-indazol-7-yl)piperidin-4-yl)ethyl)piperazine-1-carboxylate (Isomer 1) (0.6 g).

LC-MS (ESI): m/z=539.69 [M+H]⁺.

### Preparation of 3-(1-methyl-7-(4-(1-(piperazin-1-yl)ethyl)piperidin-1-yl)-1H-indazol-3-yl)piperidine-2,6-dione hydrochloride (Isomer 1)

To a stirred solution of tert-butyl 4-(1-(1-(3-(2,6-dioxopiperidin-3-yl)-1-methyl-1H-indazol-7-yl)piperidin-4-yl)ethyl)piperazine-1-carboxylate (Isomer 1) (0.6 g, 1.11 mmol) in 1,4-dioxane (3 mL) was added 4M HCl in 1,4-dioxane (3 mL) at 0° C. under a N₂ atmosphere. The reaction was stirred at room temperature for 3 hours. The reaction mixture was concentrated under reduced pressure to remove solvent and then co-distilled with DCM to afford 3-(1-methyl-7-(4-(1-(piperazin-1-yl)ethyl)piperidin-1-yl)-1H-indazol-3-yl)piperidine-2,6-dione hydrochloride (Isomer 1) (0.40 g). LC-MS (ESI): m/z=439.57 [M+H]⁺.

### Preparation of 3-(7-(4-((S)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(7-(4-((S*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione]

A stirred solution of 3-(1-methyl-7-(4-(1-(piperazin-1-yl)ethyl)piperidin-1-yl)-1H-indazol-3-yl)piperidine-2,6-dione hydrochloride (0.2 g, 0.42 mmol) in DMSO (4.0 mL) was put under N2 atmosphere. DIPEA (0.36 mL, 2.10 mmol) was added and stirred at room temperature for 5 minutes under N2 atmosphere. (R)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.13 g, 0.29 mmol) was added and the reaction was heated to 100° C. for 3 hours. The reaction was directly purified via prep HPLC to afford 3-(7-(4-((S)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione [3-(7-(4-((S*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione](0.069 g) as an off-white solid. ¹H NMR (400 MHz, DMSO-d₆): δ 10.9 (s, 1H), 8.84 (s, 1H), 8.22 (s, 1H), 8.04 (s, 1H), 7.70 (d, 1H), 7.45 (d, 1H), 7.35 (d, 1H), 7.01 (m, 2H), 6.24 (bs, 1H), 4.58 (s, 2H), 4.41-4.33 (m, 3H), 4.30 (s, 3H), 3.56 (bs, 6H), 2.32 (s, 2H), 2.67-2.60 (m, 5H), 2.33-2.32 (m, 4H), 2.18-2.14 (d, 2H, J=4), 1.90 (d, 1H), 1.53-1.35 (m, 5H), 1.23 (bs, 3H), 0.92-0.91 (d, 3H). 0.69 (m, 1H), 0.50 (m, 2H), 0.36 (m, 1H).

LC-MS (ESI): m/z=870.6 [M+H]⁺.

### Example 8. 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione (Compound 214b)

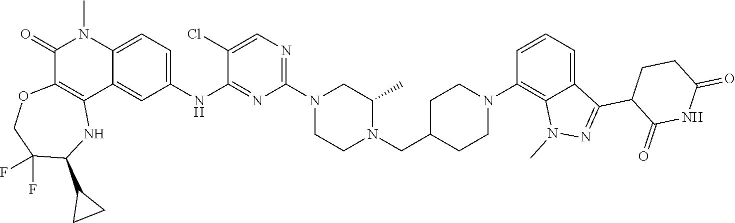 [SMILES: C[C@H]1CN(c2ncc(Cl)c(Nc3ccc4c(c3)c3c(c(=O)n4C)OCC(F)(F)[C@H](C4CC4)N3)n2)CCN1CC1CCN(c2cccc3c(C4CCC(=O)NC4=O)nn(C)c23)CC1]

### Preparation of tert-butyl (S)-4-((1-((benzyloxy)carbonyl)piperidin-4-yl)methyl)-3-methylpiperazine-1-carboxylate

To a stirred solution of tert-butyl (S)-3-methylpiperazine-1-carboxylate (10 g, 49.93 mmol) in DCM (200 mL), acetic acid (2.99 mL, 49.93 mmol) and benzyl 4-formylpiperidine-1-carboxylate (6.17 g, 24.96 mmol) were added at 0° C. After stirring for 5 hours, sodium triacetoxyborohydride (26.45 g, 124.82 mmol) was added. The reaction was allowed to warm to room temperature over 16 hours. The reaction mixture was quenched with ice-cold saturated ammonium chloride solution (200 mL) and extracted into DCM (400 mL×3). The separated organic layers were washed with brine solution (100 mL), dried over anhydrous sodium sulfate, filtered, and concentrated under reduced pressure to obtain crude tert-butyl (S)-4-((1-((benzyloxy)carbonyl)piperidin-4-yl)methyl)-3-methylpiperazine-1-carboxylate (8.0 g) as a light-yellow semi-solid. LC-MS (ESI): m/z=432.54 [M+H]⁺.

### Preparation of tert-butyl (S)-3-methyl-4-(piperidin-4-ylmethyl)piperazine-1-carboxylate

To a stirred solution of tert-butyl (S)-4-((1-((benzyloxy)carbonyl)piperidin-4-yl)methyl)-3-methylpiperazine-1-carboxylate (8.0 g, 18.53 mmol) in THF (120 mL) was added 20% Pd(OH)₂/C (4.0 g, 50% w/w). The reaction was stirred under a hydrogen atmosphere (80 psi) at room temperature for 16 hours. The reaction mixture was diluted with DCM (100 mL) and filtered through pad of celite. The celite was washed with 30% THF in DCM (300 mL) and the collected filtrate was concentrated under vacuum to obtain a crude product. The crude product was washed with n-pentane (30 mL) and dried under vacuum to obtain tert-butyl (S)-3-methyl-4-(piperidin-4-ylmethyl) piperazine-1-carboxylate (6.0 g) as a pale brown solid. LC-MS (ESI): m/z=297.24 [M+H]⁺.

### Preparation of tert-butyl (S)-4-((1-(3-(2,6-bis(benzyloxy)pyridin-3-yl)-1-methyl-1H-indazol-7-yl) piperidin-4-yl) methyl)-3-methylpiperazine-1-carboxylate

To a stirred solution of 3-(2,6-bis(benzyloxy)pyridin-3-yl)-6-bromo-1-methyl-1H-indazole (1.50 g, 2.99 mmol) in dioxane (7.5 mL) and DMF (1.5 mL) was added tert-butyl (S)-3-methyl-4-(piperidin-4-ylmethyl)piperazine-1-carboxylate (2.67 g, 8.99 mmol) and cesium carbonate (2.93 g, 8.99 mmol). The reaction was degassed with argon for 5 minutes and Pd-PEPPSI-iHeptCl (0.14 g, 0.15 mmol) was added. The reaction was heated at 100° C. for 16 hours. The reaction mixture was diluted with DCM (50 mL) and filtered through celite and washed with THF: DCM (30%) (300 mL). The collected filtrate was concentrated under vacuum to obtain crude product.

The crude product was purified by flash chromatography (230-400 silica gel; 30-27% ethyl acetate in petroleum ether) to afford tert-butyl (S)-4-((1-(3-(2,6-bis(benzyloxy)pyridin-3-yl)-1-methyl-1H-indazol-7-yl)piperidin-4-yl)methyl)-3-methylpiperazine-1-carboxylate (0.80 g) as a pale brown semi-solid. LC-MS (ESI): m/z=717.79 [M+H]⁺.

### Preparation of tert-butyl (3S)-4-((1-(3-(2,6-dioxopiperidin-3-yl)-1-methyl-1H-indazol-7-yl) piperidin-4-yl) methyl)-3-methylpiperazine-1-carboxylate

To a stirred solution of tert-butyl (S)-4-((1-(3-(2,6-bis(benzyloxy)pyridin-3-yl)-1-methyl-1H-indazol-7-yl) piperidin-4-yl)methyl)-3-methylpiperazine-1-carboxylate (0.71 g, 1.00 mmol) in THF (100 mL) was added 20% Pd(OH)₂/C (0.71 g). The reaction was put under hydrogen atmosphere (80 psi) and stirred at room temperature for 16 hours. The reaction mixture was diluted with DCM (100 mL) and filtered through celite. The celite was washed with 30% THF in DCM (300 mL) and the collected filtrate was concentrated under vacuum to obtain tert-butyl (3S)-4-((1-(3-(2,6-dioxopiperidin-3-yl)-1-methyl-1H-indazol-7-yl)piperidin-4-yl)methyl)-3-methylpiperazine-1-carboxylate (0.65 g) as a light-yellow semi-solid. LC-MS (ESI): m/z=539.41 [M+H]⁺.

### Preparation of 3-(1-methyl-7-(4-(((S)-2-methylpiperazin-1-yl)methyl)piperidin-1-yl)-1H-indazol-3-yl)piperidine-2,6-dione

To a stirred solution of tert-butyl (3S)-4-((1-(3-(2,6-dioxopiperidin-3-yl)-1-methyl-1H-indazol-7-yl) piperidin-4-yl) methyl)-3-methylpiperazine-1-carboxylate (0.650 g, 1.20 mmol) in DCM (10 mL) was added trifluoroacetic acid (5.0 mL). The reaction was stirred at room temperature for 2 hours. The reaction mixture was concentrated under vacuum to provide crude 3-(1-methyl-7-(4-(((S)-2-methylpiperazin-1-yl)methyl)piperidin-1-yl)-1H-indazol-3-yl) piperidine-2,6-dione (0.60 g) as a pale brown semi-solid. LC-MS (ESI): m/z=439.57 [M+H]⁺.

### Preparation of 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione

To a stirred solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one TFA salt (0.300 g, 0.68 mmol) in DMSO (3.2 mL, 20 V) was added DIPEA (3.2 mL, 20 V) and 3-(1-methyl-7-(4-(((S)-2-methylpiperazin-1-yl)methyl)piperidin-1-yl)-1H-indazol-3-yl)piperidine-2,6-dione (0.16 g, 0.34 mmol). The reaction was heated at 100° C. for 16 hours. The solvent was removed under vacuum to obtain a crude product. The crude product was purified by prep HPLC to obtain 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione as an off-white solid (0.063 g). 1H NMR (400 MHz, DMSO-d₆): δ 10.90 (s, 1H), 8.84 (s, 1H), 8.22 (s, 1H), 8.04 (s, 1H), 7.71 (d, J=7.20 Hz, 1H), 7.44 (d, J=9.20 Hz, 1H), 7.36 (d, J=2.00 Hz, 1H), 7.03-6.98 (m, 2H), 6.21 (s, 1H), 4.41-4.31 (m, 3H), 4.23 (s, 3H), 3.98 (d, J=12.80 Hz, 2H), 3.57 (s, 3H), 3.22-3.17 (m, 4H), 2.69-2.67 (m, 2H), 2.65-2.64 (m, 4H), 2.33-2.31 (m, 2H), 2.19-2.17 (m, 2H), 2.10-2.03 (m, 1H), 2.01-1.90 (m, 1H), 1.85-1.75 (m, 1H), 1.81-1.75 (m, 1H), 1.50-1.32 (m, 4H), 0.97 (d, J=6.00 Hz, 3H), 0.79-0.77 (m, 1H), 0.52-0.50 (m, 2H), 0.36-0.34 (m, 1H). LC-MS (ESI): m/z=868.44 [M−H]⁻.

### Example 9. 3-(7-(4-((R)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(7-(4-((R*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione](Compound 380b)

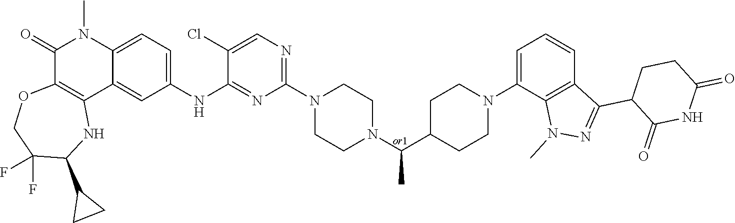 [SMILES: C[C@H](C1CCN(c2cccc3c(C4CCC(=O)NC4=O)nn(C)c23)CC1)N1CCN(c2ncc(Cl)c(Nc3ccc4c(c3)c3c(c(=O)n4C)OCC(F)(F)[C@H](C4CC4)N3)n2)CC1]

### Preparation of tert-butyl 4-(1-(piperidin-4-yl)ethyl)piperazine-1-carboxylate (Isomer 2)

A stirred solution of tert-butyl 4-(1-(1-((benzyloxy)carbonyl)piperidin-4-yl)ethyl)piperazine-1-carboxylate (Isomer 2) (1.5 g, 3.47 mmol) in THF (30 mL) was put under N₂ atmosphere.

Palladium on carbon, 10% Pd (Dry) (0.3 g, 20% w/w) was added and the resultant reaction mixture was stirred under hydrogen pressure (50 psi) at room temperature for 16 hours. The reaction mixture was filtered through celite and the celite was washed with ethyl acetate and THF. The combined organic layers were concentrated to obtain tert-butyl 4-(1-(piperidin-4-yl)ethyl)piperazine-1-carboxylate (Isomer 2) as a brown sticky solid (1.0 g). LC-MS (ESI): m/z=298.20 [M+H]⁺.

### Preparation of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 2)

A stirred solution of 3-(2,6-bis(benzyloxy)pyridin-3-yl)-7-bromo-1-methyl-1H-indazole (1.4 g, 2.80 mmol) and tert-butyl 4-(1-(piperidin-4-yl)ethyl)piperazine-1-carboxylate (Isomer 2) (1.0 g, 3.35 mmol) in 1,4-dioxane (28 mL) was put under N2 atmosphere. Cesium carbonate (2.73 g, 8.39 mmol) was added and the reaction was degassed with N2 for 10 minutes. Pd-PEPPSI-iHEPTCl (0.14 g, 0.14 mmol) was added and the resulting reaction mixture was heated to 100° C. for 16 hours. The reaction was filtered through celite and the celite was washed with ethyl acetate. The combined organic layers were washed with cold brine, dried over anhydrous sodium sulfate, filtered, and concentrated to afford a crude product. The crude product was purified by flash column chromatography (SiO₂, 100-200, 18% ethyl acetate in petroleum ether) to afford tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 2) (1.4 g) as an off-white solid. LC-MS (ESI): m/z=717.97 [M+H]⁺.

### Preparation of tert-butyl 4-(1-(1-(3-(2,6-dioxopiperidin-3-yl)-1-methyl-1H-indazol-7-yl)piperidin-4-yl)ethyl)piperazine-1-carboxylate (Isomer 2)

A stirred solution of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 2) (1.0 g, 1.40 mmol) in THF (40 mL) was put under N2 atmosphere. Pd(OH)₂ on carbon, 20% Pd (0.50 g, 50% w/w) was added, and the reaction was stirred under hydrogen atmosphere with balloon pressure at room temperature for 16 hours. The reaction was filtered through celite and the celite was washed with ethyl acetate. The combined organic layers were concentrated to obtain a crude product. The crude product was purified by triturating with n-hexane to afford tert-butyl 4-(1-(1-(3-(2,6-dioxopiperidin-3-yl)-1-methyl-1H-indazol-7-yl)piperidin-4-yl)ethyl)piperazine-1-carboxylate (Isomer 2) (0.7 g) as an off white solid. LC-MS (ESI): m/z=539.63 [M+H]⁺.

### Preparation of 3-(1-methyl-7-(4-(1-(piperazin-1-yl)ethyl)piperidin-1-yl)-1H-indazol-3-yl)piperidine-2,6-dione hydrochloride (Isomer 2)

To a stirred solution tert-butyl 4-(1-(1-(3-(2,6-dioxopiperidin-3-yl)-1-methyl-1H-indazol-7-yl)piperidin-4-yl)ethyl)piperazine-1-carboxylate (Isomer 2) (0.70 g, 1.30 mmol) in DCM (7.0 mL) was added 4M HCl in dioxane (3.50 mL) at 0° C. under N2 atmosphere. The reaction was stirred at room temperature for 5 hours. The reaction was concentrated under reduced pressure and co-distilled with DCM to obtain a crude product. The crude product was purified by triturating with diethyl ether to afford 3-(1-methyl-7-(4-(1-(piperazin-1-yl)ethyl)piperidin-1-yl)-1H-indazol-3-yl)piperidine-2,6-dione hydrochloride (Isomer 2) (0.84 g) as a white solid. LC-MS (ESI): m/z=439.65 [M+H]⁺.

### Preparation of 3-(7-(4-((R)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(7-(4-((R*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione]

A stirred solution of 3-(1-methyl-7-(4-(1-(piperazin-1-yl)ethyl)piperidin-1-yl)-1H-indazol-3-yl)piperidine-2,6-dione hydrochloride (Isomer 2) (0.20 g, 0.42 mmol) in DMSO (4.0 mL) was put under nitrogen atmosphere and cooled to 0° C. DIPEA (0.59 mL, 3.40 mmol) was added and stirred at room temperature for 5 minutes under N2 atmosphere. (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.21 mmol) was added and the resulting reaction mixture was heated at 100° C. for 7 hours. The reaction was directly purified by prep HPLC to afford 3-(7-(4-((R)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione [3-(7-(4-((R*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione](0.094 g) as an off-white solid. 1H NMR (400 MHz, DMSO-d₆): δ 10.84 (s, 1H), 8.83 (s, 1H), 8.24 (s, 1H), 8.04 (s, 1H), 7.70-7.67 (dd, J=9.2 Hz, j=2.0 Hz, 1H), 7.45-7.42 (d, J=8.8 Hz, 1H), 7.37-7.35 (d, J=7.2 Hz, 1H), 7.03-6.97 (m, 2H), 6.18 (s, 1H), 4.46-4.30 (m, 3H), 4.23 (s, 3H), 3.59-3.56 (m, 7H), 3.24 (s, 3H), 2.67-2.54 (m, 7H), 2.32-2.30 (m, 4H), 2.18-2.14 (m, 2H), 1.82-1.79 (m, 1H), 1.38-1.31 (m, 4H), 0.90-0.89 (d, J=6.4 Hz, 3H), 0.69 (m, 1H), 0.52-0.50 (m, 2H), 0.36-0.34 (m, 1H). LC-MS (ESI): m/z=870.57 [M+H]⁺.

### Example 10. 3-(6-(4-((S)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(6-(4-((S*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione](Compound 379a)

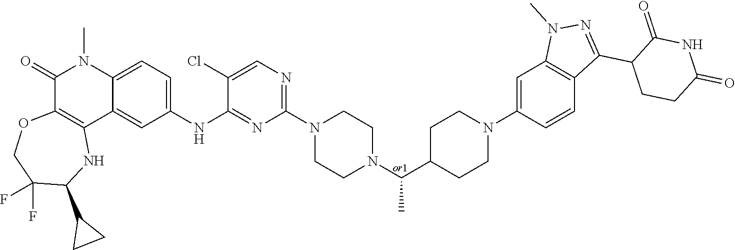 [SMILES: C[C@@H](C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)[C@H](C4CC4)N3)n2)CC1]

### Preparation of tert-butyl 4-(1-(piperidin-4-yl)ethyl)piperazine-1-carboxylate

To a stirred solution of tert-butyl 4-(1-(1-((benzyloxy)carbonyl)piperidin-4-yl)ethyl)piperazine-1-carboxylate (10 g, 23.19 mmol) in THF (200 mL) was added palladium on carbon, 10% Pd (Dry) (2.0 g, 20% w/w). The reaction mixture was stirred under hydrogen pressure (50 psi) at room temperature for 16 hours. The reaction mixture was filtered through celite and the celite was washed with ethyl acetate. The combined organic layers were concentrated to afford tert-butyl 4-(1-(piperidin-4-yl)ethyl)piperazine-1-carboxylate as an off-white sticky solid (5.5 g). LC-MS (ESI): m/z=298.24 [M+H]⁺.

### Preparation of tert-butyl 4-(1-(1-(3-(2,6-bis(benzyloxy)pyridin-3-yl)-1-methyl-1H-indazol-6-yl)piperidin-4-yl)ethyl)piperazine-1-carboxylate (Isomers 1 and 2)

A stirred solution of tert-butyl 4-(1-(piperidin-4-yl)ethyl)piperazine-1-carboxylate (1.2 g, 4.03 mmol) and 3-(2,6-bis(benzyloxy)pyridin-3-yl)-6-bromo-1-methyl-1H-indazole (1.41 g, 2.80 mmol) in 1,4-dioxane (24 mL) was put under N₂ atmosphere. Potassium tert-butoxide (1.36 g, 12.10 mmol) was added and the reaction was degassed with N2 for 10 minutes. t-BuBrettPhos Pd G3 (0.17 g, 0.20 mmol) was added, and the reaction was stirred at 100° C. for 16 hours. The reaction was filtered through celite and the celite was washed with ethyl acetate. The combined organic layers were concentrated to obtain a crude product. The crude product was purified by flash column chromatography (SiO₂, 100-200, 15% ethyl acetate in petroleum ether) to afford tert-butyl 4-(1-(1-(3-(2,6-bis(benzyloxy)pyridin-3-yl)-1-methyl-1H-indazol-6-yl)piperidin-4-yl)ethyl)piperazine-1-carboxylate. The enantiomers were separated via chiral HPLC SFC purification to afford tert-butyl 4-(1-(1-(3-(2,6-bis(benzyloxy)pyridin-3-yl)-1-methyl-1H-indazol-6-yl)piperidin-4-yl)ethyl)piperazine-1-carboxylate (Isomer-1) as an off-white solid (0.18 g) and tert-butyl 4-(1-(1-(3-(2,6-bis(benzyloxy)pyridin-3-yl)-1-methyl-1H-indazol-6-yl)piperidin-4-yl)ethyl)piperazine-1-carboxylate (Isomer-2) as an off-white solid (0.20 g). LC-MS (ESI) Isomer 1: m/z=717.83 [M+H]⁺; LC-MS (ESI) Isomer 2: m/z=717.79 [M+H]⁺.

### Preparation of tert-butyl 4-(1-(1-(3-(2,6-dioxopiperidin-3-yl)-1-methyl-1H-indazol-6-yl)piperidin-4-yl)ethyl)piperazine-1-carboxylate (Isomer 1)

A stirred solution of tert-butyl 4-(1-(1-(3-(2,6-bis(benzyloxy)pyridin-3-yl)-1-methyl-1H-indazol-6-yl)piperidin-4-yl)ethyl)piperazine-1-carboxylate (Isomer 1) (0.18 g, 0.25 mmol) in THF (9 mL) was put under N2 atmosphere. Pd(OH)₂ on carbon, 20% Pd (0.09 g, 50% w/w) was added, and the reaction mixture was stirred under hydrogen atmosphere at 40 psi for 16 hours. The reaction mixture was filtered through celite and the celite was washed with ethyl acetate. The combined organic layers were concentrated to provide a crude product. The crude product was purified by triturating with n-hexane to afford tert-butyl 4-(1-(1-(3-(2,6-dioxopiperidin-3-yl)-1-methyl-1H-indazol-6-yl)piperidin-4-yl)ethyl)piperazine-1-carboxylate (Isomer 1) as an off-white solid (0.13 g). LC-MS (ESI): m/z=539.65 [M+H]⁺.

### Preparation of 3-(1-methyl-6-(4-(1-(piperazin-1-yl)ethyl)piperidin-1-yl)-1H-indazol-3-yl)piperidine-2,6-dione hydrochloride (Isomer 1)

To a stirred solution of tert-butyl 4-(1-(1-(3-(2,6-dioxopiperidin-3-yl)-1-methyl-1H-indazol-6-yl)piperidin-4-yl)ethyl)piperazine-1-carboxylate (Isomer 1) (0.13 g, 0.24 mmol) in DCM (1.3 mL) was added 4M HCl in 1,4-dioxane (0.65 mL) at 0° C. under N2 atmosphere. The reaction mixture was stirred at room temperature for 5 hours. The reaction mixture was concentrated under reduced pressure and remaining solvent was co-distilled with DCM to afford a crude product. The crude product was purified by triturating with diethyl ether to afford 3-(1-methyl-6-(4-(1-(piperazin-1-yl)ethyl)piperidin-1-yl)-1H-indazol-3-yl)piperidine-2,6-dione hydrochloride (Isomer 1) as an off-white solid (0.11 g). LC-MS (ESI): m/z=439.53 [M+H]⁺.

### Preparation of 3-(6-(4-((S)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(6-(4-((S*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione]

A stirred solution of 3-(1-methyl-6-(4-(1-(piperazin-1-yl)ethyl)piperidin-1-yl)-1H-indazol-3-yl)piperidine-2,6-dione hydrochloride (Isomer 1) (0.11 g, 0.23 mmol) in DMSO (3.3 mL) was put under N2 atmosphere and cooled to 0° C. DIPEA (0.32 mL, 1.85 mmol) was added and the reaction was allowed to warm for 5 minutes under N2 atmosphere. (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.055 g, 0.12 mmol) was added and the reaction mixture was heated to 120° C. for 6 hours. The crude reaction was purified by prep HPLC to afford 3-(6-(4-((S)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione [3-(6-(4-((S*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione] as an off-white solid (0.068 g). 1H NMR (400 MHz, DMSO-d₆): δ 10.84 (s, 1H), 8.83 (s, 1H), 8.24 (s, 2H), 8.04 (s, 1H), 7.70-7.67 (dd, J=9.2 Hz, J=2.0 Hz, 1H), 7.48-7.42 (q, J=13.6 Hz, J=9.2 Hz, 2H), 6.90-6.88 (d, J=9.2 Hz, 1H), 6.80 (s, 1H), 6.19 (s, 1H), 4.40 (m, 2H), 4.26-4.22 (dd, J=9.2 Hz, j=5.2 Hz, 1H), 3.87 (s, 3H), 3.79 (s, 2H), 3.58-3.55 (m, 7H), 3.31 (s, 1H), 2.67-2.59 (m, 7H), 2.33-2.28 (m, 3H), 2.17-2.07 (m, 2H), 1.76-1.73 (m, 1H), 1.29-1.23 (m, 4H), 0.88-0.87 (d, J=6.4 Hz, 3H), 0.71 (m, 1H), 0.52-0.50 (t, J=10.8 Hz, J=5.6 Hz, 2H), 0.36-0.35 (m, 1H). LC-MS (ESI): m/z=868.44 [M−H]⁺.

### Example 11. 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione (Compound 381a)

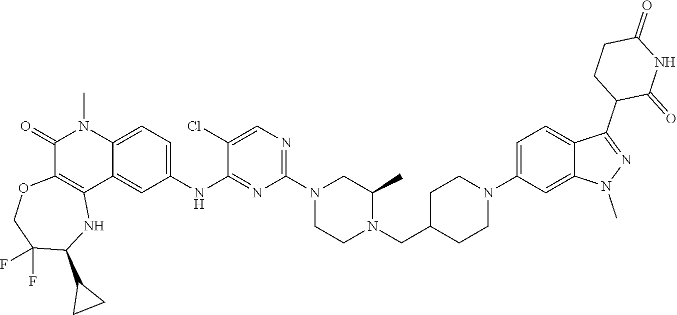 [SMILES: C[C@@H]1CN(c2ncc(Cl)c(Nc3ccc4c(c3)c3c(c(=O)n4C)OCC(F)(F)[C@H](C4CC4)N3)n2)CCN1CC1CCN(c2ccc3c(C4CCC(=O)NC4=O)nn(C)c3c2)CC1]

### Preparation of tert-butyl (R)-4-((1-(3-(2,6-bis(benzyloxy)pyridin-3-yl)-1-methyl-1H-indazol-6-yl)piperidin-4-yl)methyl)-3-methylpiperazine-1-carboxylate

To a stirred solution 3-(2,6-bis(benzyloxy)pyridin-3-yl)-6-bromo-1-methyl-1H-indazole (1.5 g, 2.99 mmol) in 1,4-dioxane (30 mL) was added tert-butyl (R)-3-methyl-4-(piperidin-4-ylmethyl)piperazine-1-carboxylate (3.5 g, 11.99 mmol). The reaction was sparged with argon for 15 minutes, sodium tert-butoxide (0.86 g, 8.97 mmol) and BrettPhos Pd G3 (0.13 g, 0.05 mmol) were added. The reaction was then heated at 80° C. for 16 hours. The reaction mixture was quenched with water and extracted into ethyl acetate. The combined organic layers were washed with brine solution, dried over anhydrous sodium sulfate, filtered, and concentrated under vacuum to provide a crude product. The crude product was purified by flash column chromatography (90% ethyl acetate in petroleum ether) to afford tert-butyl (R)-4-((1-(3-(2,6-bis(benzyloxy)pyridin-3-yl)-1-methyl-1H-indazol-6-yl)piperidin-4-yl)methyl)-3-methylpiperazine-1-carboxylate (0.7 g) as a yellow gummy solid. LC-MS (ESI): m/z=717.79 [M+H]⁺.

### Preparation of tert-butyl (3R)-4-((1-(3-(2,6-dioxopiperidin-3-yl)-1-methyl-1H-indazol-6-yl)piperidin-4-yl)methyl)-3-methylpiperazine-1-carboxylate

To a stirred solution of tert-butyl (R)-4-((1-(3-(2,6-bis(benzyloxy)pyridin-3-yl)-1-methyl-1H-indazol-6-yl)piperidin-4-yl)methyl)-3-methylpiperazine-1-carboxylate (0.70 g, 0.97 mmol) in THF (14 mL) was added 20% Pd(OH)₂ on carbon, moisture 50% wet (0.68 g, 4.88 mmol). The reaction was put under hydrogen atmosphere (60 psi) at room temperature and stirred for 16 hours.

The reaction mixture was diluted with DCM and filtered through celite, and the celite was washed with 30% THF in DCM. The collected filtrate was concentrated under vacuum to obtain crude tert-butyl (3R)-4-((1-(3-(2,6-dioxopiperidin-3-yl)-1-methyl-1H-indazol-6-yl)piperidin-4-yl)methyl)-3-methylpiperazine-1-carboxylate (0.4 g) as a light yellow gummy solid. LC-MS (ESI): m/z=539.59 [M+H]⁺.

### Preparation of 3-(1-methyl-6-(4-(((R)-2-methylpiperazin-1-yl)methyl)piperidin-1-yl)-1H-indazol-3-yl)piperidine-2,6-dione

To a stirred solution of tert-butyl (3R)-4-((1-(3-(2,6-dioxopiperidin-3-yl)-1-methyl-1H-indazol-6-yl)piperidin-4-yl)methyl)-3-methylpiperazine-1-carboxylate (0.40 g, 0.74 mmol) in DCM (4 mL) was added trifluoroacetic acid (4 mL). The reaction was stirred at room temperature for 2 hours. The reaction mixture was concentrated under vacuum to obtain 3-(1-methyl-6-(4-(((R)-2-methylpiperazin-1-yl)methyl)piperidin-1-yl)-1H-indazol-3-yl)piperidine-2,6-dione (0.38 g) as a pale yellow gummy solid. LC-MS (ESI): m/z=439.53 [M+H]⁺.

### Preparation of 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione

To a stirred solution of 3-(1-methyl-6-(4-(((R)-2-methylpiperazin-1-yl)methyl)piperidin-1-yl)-1H-indazol-3-yl)piperidine-2,6-dione (0.20 g, 0.45 mmol) in DMSO (2.0 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.07 g, 0.18 mmol) and DIPEA (0.65 mL, 4.0 mmol).

The reaction was heated at 100° C. for 6 hours. The reaction mixture was concentrated under vacuum to obtain a crude product. The crude product was purified by prep HPLC to afford 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione (13.3 mg) as a pale brown solid. 1H NMR (400 MHz, DMSO-d₆): δ 10.85 (s, 1H), 9.44-9.06 (m, 2H), 8.20 (s, 1H), 8.13 (d, J=9.20 Hz, 1H), 7.67 (d, J=7.60 Hz, 1H), 7.51-7.44 (m, 2H), 6.96-6.85 (m, 1H), 6.25 (s, 1H), 4.44-4.43 (m, 3H), 4.27-4.24 (m, 1H), 4.07-3.83 (m, 3H), 3.57-3.26 (m, 3H), 3.13-3.01 (m, 7H), 2.76-2.67 (m, 2H), 2.63-2.59 (m, 2H), 2.31 (s, 1H), 2.17 (s, 1H), 1.91 (m, 3H), 1.41-1.36 (m, 1H), 1.30-1.20 (m, 9H), 0.72-0.70 (m, 1H), 0.53 (br s, 2H), 0.34 (br s, 1H). LC-MS (ESI): m/z=868.7 [M−H]⁻.

### Example 12. 3-(6-(4-((R)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(6-(4-((R*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione](Compound 379b)

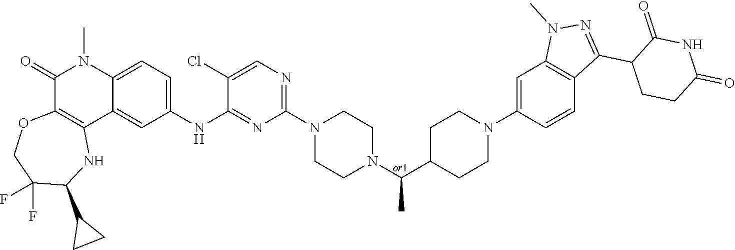 [SMILES: C[C@H](C1CCN(c2ccc3c(C4CCC(=O)NC4=O)nn(C)c3c2)CC1)N1CCN(c2ncc(Cl)c(Nc3ccc4c(c3)c3c(c(=O)n4C)OCC(F)(F)[C@H](C4CC4)N3)n2)CC1]

### Preparation of tert-butyl 4-(1-(1-(3-(2,6-dioxopiperidin-3-yl)-1-methyl-1H-indazol-6-yl)piperidin-4-yl)ethyl)piperazine-1-carboxylate (Isomer 2)

A stirred solution of tert-butyl 4-(1-(1-(3-(2,6-bis(benzyloxy)pyridin-3-yl)-1-methyl-1H-indazol-6-yl)piperidin-4-yl)ethyl)piperazine-1-carboxylate (Isomer 2) (0.2 g, 0.27 mmol) in THF (10 mL) was put under N2 atmosphere. Pd(OH)₂ on carbon, 20% Pd (0.1 g, 50% w/w) was added, and the reaction mixture was stirred under hydrogen atmosphere with balloon pressure at room temperature for 16 hours. The reaction mixture was filtered through celite and the celite washed with ethyl acetate. The combined organic layers were concentrated to provide a crude product. The crude product was purified by triturating with n-hexane to afford tert-butyl 4-(1-(1-(3-(2,6-dioxopiperidin-3-yl)-1-methyl-1H-indazol-6-yl)piperidin-4-yl)ethyl)piperazine-1-carboxylate (Isomer 2) as an off-white solid (0.15 g). LC-MS (ESI): m/z=539.61 [M+H]⁺.

### Preparation of 3-(1-methyl-6-(4-(1-(piperazin-1-yl)ethyl)piperidin-1-yl)-1H-indazol-3-yl)piperidine-2,6-dione hydrochloride (Isomer 2)

To a stirred solution of tert-butyl 4-(1-(1-(3-(2,6-dioxopiperidin-3-yl)-1-methyl-1H-indazol-6-yl)piperidin-4-yl)ethyl)piperazine-1-carboxylate (Isomer 2) (0.15 g, 0.27 mmol) in DCM (1.5 mL) was added 4M HCl in 1,4-dioxane (0.75 mL) at 0° C. under N2 atmosphere. The reaction was stirred at room temperature for 5 hours. The reaction mixture was concentrated under reduced pressure and excess solvent was co-distilled with DCM. The crude product was purified by triturating with diethyl ether to afford 3-(1-methyl-6-(4-(1-(piperazin-1-yl)ethyl)piperidin-1-yl)-1H-indazol-3-yl)piperidine-2,6-dione hydrochloride (Isomer 2) as an off-white solid (0.12 g). LC-MS (ESI): m/z=439.53 [M+H]⁺.

### Preparation of 3-(6-(4-((R)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

### [3-(6-(4-((R*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione]

A stirred solution of 3-(1-methyl-6-(4-(1-(piperazin-1-yl)ethyl)piperidin-1-yl)-1H-indazol-3-yl)piperidine-2,6-dione hydrochloride (Isomer 2) (0.12 g, 0.25 mmol) in DMSO (3.6 mL) was cooled to 0° C. under N2 atmosphere. DIPEA (0.35 mL, 2.02 mmol) was added and the reaction was allowed to warm to room temperature over 5 minutes under N2 atmosphere. (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.13 mmol) was added and the resulting reaction mixture was heated to 120° C. for 6 hours. The crude reaction was directly purified by prep HPLC to afford 3-(6-(4-((R)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione [3-(6-(4-((R*)-1-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione] as an off-white solid (0.083 g). 1H NMR (400 MHz, DMSO-d₆): δ 10.84 (s, 1H), 8.83 (s, 1H), 8.21 (s, 2H), 8.04 (s, 1H), 7.71-7.68 (dd, J=9.2 Hz, J=2.0 Hz, 1H), 7.48-7.42 (q, J=13.6 Hz, J=9.2 Hz, 2H), 6.90-6.88 (d, J=9.2 Hz, 1H), 6.80 (s, 1H), 6.23 (s, 1H), 4.40 (m, 2H), 4.26-4.22 (dd, J=9.2 Hz, j=5.2 Hz, 1H), 3.87 (s, 3H), 3.79 (s, 2H), 3.55 (s, 7H), 3.31 (s, 1H), 2.67-2.56 (m, 7H), 2.32-2.28 (m, 3H), 2.17-2.11 (m, 2H), 1.73 (s, 1H), 1.31-1.23 (m, 4H), 0.88-0.86 (d, J=6.4 Hz, 3H), 0.72-0.70 (m, 1H), 0.52-0.50 (t, J=10.8 Hz, J=5.6 Hz, 2H), 0.36-0.35 (m, 1H). LC-MS (ESI): m/z=870.53 [M+H]⁺.

### Example 13. 3-(6-(((1s,4r)-4-(((5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)amino)methyl)cyclohexyl)amino)-1-methyl-1H-indazol-3-yl)piperidine-2,6-dione (Compound 390a)

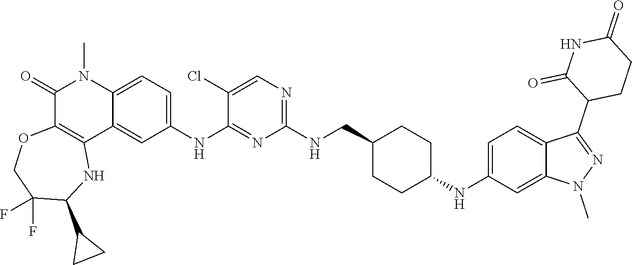 [SMILES: Cn1nc(C2CCC(=O)NC2=O)c2ccc(N[C@H]3CC[C@H](CNc4ncc(Cl)c(Nc5ccc6c(c5)c5c(c(=O)n6C)OCC(F)(F)[C@H](C6CC6)N5)n4)CC3)cc21]

### Preparation of tert-butyl (((1r,4r)-4-((3-(2,6-bis(benzyloxy)pyridin-3-yl)-1-methyl-1H-indazol-6-yl)amino)cyclohexyl)methyl)carbamate

To a stirred solution of tert-butyl (((1r,4r)-4-aminocyclohexyl)methyl)carbamate (0.500 g, 2.190 mmol) and 3-(2,6-bis(benzyloxy)pyridin-3-yl)-6-bromo-1-methyl-1H-indazole (0.760 g, 1.53 mmol) in 1,4-dioxane (5 mL) was added sodium tert-butoxide, 98%, pure (0.420 g, 4.380 mmol).

The reaction mixture was purged with N2 for 5 minutes. tBuBrettPhos Pd G3 (0.93 g, 0.109 mmol) was added, and the reaction was heated at 100° C. for 2 hours. The reaction mixture was quenched with ice cold water (50 mL) and extracted with ethyl acetate. The combined organic layers were washed with brine solution (50 mL) and dried over anhydrous sodium sulfate, filtered, and concentrated to provide a crude product. The crude product was purified by flash column chromatography (30% ethyl acetate in petroleum ether) to afford tert-butyl (((1r,4r)-4-((3-(2,6-bis(benzyloxy)pyridin-3-yl)-1-methyl-1H-indazol-6-yl)amino)cyclohexyl)methyl)carbamate (0.600 g) as a brown sticky solid. LC-MS (ESI): m/z=648.65 [M+H]⁺.

### Preparation of tert-butyl (((1r,4r)-4-((3-(2,6-dioxopiperidin-3-yl)-1-methyl-1H-indazol-6-yl)amino)cyclohexyl)methyl)carbamate

A stirred solution of tert-butyl (((1r,4r)-4-((3-(2,6-bis(benzyloxy)pyridin-3-yl)-1-methyl-1H-indazol-6-yl)amino)cyclohexyl)methyl)carbamate (0.500 g, 0.772 mmol) in THF (10 mL) was purged with nitrogen for 5 minutes. After adding 20% Pd(OH)₂ on carbon (0.488 g, 3.473 mmol), the reaction mixture was stirred at room temperature for 16 hours under H₂ gas balloon atmosphere. The reaction mixture was filtered through celite, the celite was washed with ethyl acetate, and the filtrate was concentrated to provide a crude product. The crude product was purified by trituration with diethyl ether to afford tert-butyl (((1r,4r)-4-((3-(2,6-dioxopiperidin-3-yl)-1-methyl-1H-indazol-6-yl)amino)cyclohexyl)methyl)carbamate (0.350 g) as a light brown solid. LC-MS (ESI): m/z=470.31 [M+H]⁺.

### Preparation of 3-(6-(((1r,4r)-4-(aminomethyl)cyclohexyl)amino)-1-methyl-1H-indazol-3-yl)piperidine-2,6-dione hydrochloride

To a stirred solution of tert-butyl (((1r,4r)-4-((3-(2,6-dioxopiperidin-3-yl)-1-methyl-1H-indazol-6-yl)amino)cyclohexyl)methyl)carbamate (0.350 g, 0.426 mmol) in DCM (3.5 mL) was added 4M HCl in 1,4-dioxane (3.5 mL) at 0° C. under N₂ atmosphere. The reaction mixture was allowed to warm to room temperature over 2 hours. Solvent was evaporated under reduced pressure to provide a crude product. The crude product was purified by trituration with diethyl ether to afford 3-(6-(((1r,4r)-4-(aminomethyl)cyclohexyl)amino)-1-methyl-1H-indazol-3-yl)piperidine-2,6-dione hydrochloride as off-white solid (0.300 g). LC-MS (ESI): m/z=370.42 [M+H]⁺.

### Preparation of 3-(6-(((1s,4r)-4-(((5-chloro-4-(((S)-2-cyclopropyl-3,3-difluoro-7-methyl-6-oxo-1,2,3,4,6,7-hexahydro-[1,4]oxazepino[2,3-e]quinoline-O-yl)amino)pyrimidin-2-yl)amino)methyl)cyclohexyl)amino)-1-methyl-1H-indazol-3-yl)piperidine-2,6-dione

To stirred solution of 3-(6-(((1r,4r)-4-(aminomethyl)cyclohexyl)amino)-1-methyl-1H-indazol-3-yl)piperidine-2,6-dione hydrochloride (0.280 g, 0.690 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.226 g, 0.483 mmol) in DMSO (2.8 mL), DIPEA (0.61 mL, 3.349 mmol) and pTSA (0.012 g, 0.069 mmol) were added. The reaction was stirred at 130° C. for 3 hours. The reaction mixture was then quenched with ice cold water (10 mL). The resulting solid was filtered off, washed with ice-cold water, and dried under vacuum to afford a crude product. The crude product was purified by prep HPLC to afford 3-(6-(((1s,41r)-4-(((5-chloro-4.—(((S)-2-cyclopropyl-3,3-difluoro-7-methyl-6-oxo-1,2,3,4,6,7-hexahydro-[1,4]oxazepino[2,3-c]quinin-10-yl)amino)pyrimidin-2-yl)amino)methyl)cyclohexyl)amino)-methyl-1-1-indazol-3-yl)piperidine-2,6-dione (0.042 g) as an off-white solid. ¹H NMR (400 MHz, DMSO): δ 10.80 (s, 1H), 8.67 (s, 1H), 8.25 (s, 3H), 7.95 (s, 1H), 7.82 (d, J=6.8 Hz, 1H), 7.41 (d, J=8.8 Hz, 1H), 7.03 (s, 1H), 6.46 (d, J=8.40 Hz, 1H), 6.31 (s, 1H), 6.21 (s, 1H), 5.62 (s, 1H), 4.34-4.36 (m, 1H), 4.16 (q, J=5.2 Hz, 1H), 3.79 (s, 3H), 3.56 (s, 3H), 3.22-3.25 (m, 4H), 2.59 (t, J=6.00 Hz, 2H), 2.22-2.23 (m, 1H), 2.13-2.15 (m, 1H), 1.99 (s, 2H), 1.33-1.35 (m, 2H), 1.02 (s, 1H), 1.13-1.14 (m, 1H), 0.9-1.1 (m, 4H), 0.7 (t, J=5.60 Hz, 1H), 0.52 (t, J=5.60 Hz, 2H), 0.357 (s, 1H). LC-MS (ESI): m/z=801.36 [M+H]⁺.

### Example 14. 3-(7-(8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,8-diazaspiro[5.5]undecan-2-yl)-1-methyl-1H-indazol-3-yl)piperidine-2,6-dione (Compound 399a)

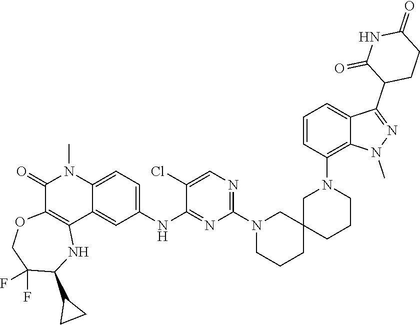 [SMILES: Cn1nc(C2CCC(=O)NC2=O)c2cccc(N3CCCC4(CCCN(c5ncc(Cl)c(Nc6ccc7c(c6)c6c(c(=O)n7C)OCC(F)(F)[C@H](C7CC7)N6)n5)C4)C3)c21]

### Preparation of tert-butyl 8-(3-(2,6-bis(benzyloxy)pyridin-3-yl)-1-methyl-1H-indazol-7-yl)-2,8-diazaspiro[5.5]undecane-2-carboxylate

To a stirred solution of 3-(2,6-bis(benzyloxy)pyridin-3-yl)-7-bromo-1-methyl-1H-indazole (0.50 g, 0.99 mmol) and tert-butyl 2,8-diazaspiro[5.5]undecane-2-carboxylate (0.30 g, 1.19 mmol) in 1,4-dioxane (20 mL) was added cesium carbonate (0.97 g, 2.99 mmol). The reaction was degassed with argon for 10 minutes, Pd-PEPPSI-iHeptCl (0.049 g, 0.05 mmol) was added, and then heated at 100° C. for 12 hours. The reaction mixture was filtered through celite and concentrated to obtain a crude product. The crude product was purified by flash column chromatography (30% ethyl acetate in petroleum ether) to afford tert-butyl 8-(3-(2,6-bis(benzyloxy)pyridin-3-yl)-1-methyl-1H-indazol-7-yl)-2,8-diazaspiro[5.5]undecane-2-carboxylate (0.52 g) as a pale yellow semi-solid.

LC-MS (ESI): m/z=674.78 [M+H]⁺.

### Preparation of tert-butyl 8-(3-(2,6-dioxopiperidin-3-yl)-1-methyl-1H-indazol-7-yl)-2,8-diazaspiro[5.5]undecane-2-carboxylate

To a stirred solution of tert-butyl 8-(3-(2,6-bis(benzyloxy)pyridin-3-yl)-1-methyl-1H-indazol-7-yl)-2,8-diazaspiro[5.5]undecane-2-carboxylate (0.90 g, 1.3 mmol) in THF (18 mL) was added 20% Pd(OH)₂ on carbon (0.90 g). The reaction was put under hydrogen atmosphere (60 psi) and stirred at room temperature for 6 hours. The reaction mixture was diluted with DCM (20 mL) and filtered through celite. The collected filtrate was concentrated under vacuum to afford tert-butyl 8-(3-(2,6-dioxopiperidin-3-yl)-1-methyl-1H-indazol-7-yl)-2,8-diazaspiro[5.5]undecane-2-carboxylate (0.65 g) as a pale brown semi-solid. LC-MS (ESI): m/z=496.60 [M+H]⁺.

### Preparation of 3-(1-methyl-7-(2,8-diazaspiro[5.5]undecan-2-yl)-1H-indazol-3-yl)piperidine-2,6-dione

To a stirred solution of tert-butyl 8-(3-(2,6-dioxopiperidin-3-yl)-1-methyl-1H-indazol-7-yl)-2,8-diazaspiro[5.5]undecane-2-carboxylate (0.65 g, 1.31 mmol) in DCM (7 mL) was added TFA (1.9 mL). The reaction was stirred at room temperature for 3 hours. The reaction mixture was concentrated to obtain a crude product. The crude product was co-distilled with petroleum ether to obtain 3-(1-methyl-7-(2,8-diazaspiro[5.5]undecan-2-yl)-1H-indazol-3-yl)piperidine-2,6-dione as a brown solid (0.52 g, TFA salt). LC-MS (ESI): m/z=396.50 [M+H]⁺.

### Preparation of 3-(7-(8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,8-diazaspiro[5.5]undecan-2-yl)-1-methyl-1H-indazol-3-yl)piperidine-2,6-dione

To a stirred solution of 3-(1-methyl-7-(2,8-diazaspiro[5.5]undecan-2-yl)-1H-indazol-3-yl)piperidine-2,6-dione (0.25 g, 0.63 mmol) in DMSO (5 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.11 g, 0.25 mmol) and DIPEA (1.1 mL, 6.3 mmol). The reaction was stirred at 100° C. for 12 hours. The reaction mixture was concentrated under vacuum to obtain a crude product. The crude product was purified by prep HPLC to afford 3-(7-(8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,8-diazaspiro[5.5]undecan-2-yl)-1-methyl-1H-indazol-3-yl)piperidine-2,6-dione (0.078 mg) as a yellow solid. ¹H NMR 400 MHz, DMSO-d₆: δ 10.87 (s, 1H), 8.83-8.74 (m, 1H), 8.18 (br s, 1H), 8.01-7.68 (m, 2H), 7.42-7.35 (m, 2H), 6.99-6.86 (m, 2H), 6.26-6.20 (m, 1H), 4.33-4.28 (m, 5H), 4.22-4.21 (br, 1H), 3.83-3.70 (br s, 4H), 3.33-3.22 (br, 4H), 2.93 (br, 1H), 2.67-2.58 (m, 4H), 2.39-2.32 (m, 2H), 1.99-1.98 (m, 1H), 1.51-1.11 (m, 9H), 0.70 (br s, 1H), 0.50 (br s, 2H), 0.32 (br s, 1H). LC-MS (ESI): m/z=827.54 [M+H]⁺.

### Example 15. 3-(6-(6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.5]nonan-2-yl)-1-methyl-1H-indazol-3-yl)piperidine-2,6-dione (Compound 396a)

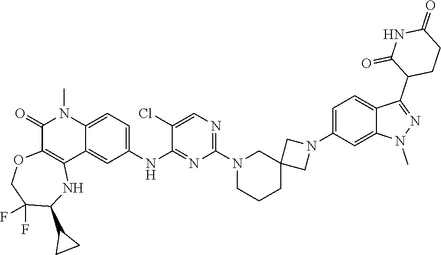 [SMILES: Cn1nc(C2CCC(=O)NC2=O)c2ccc(N3CC4(CCCN(c5ncc(Cl)c(Nc6ccc7c(c6)c6c(c(=O)n7C)OCC(F)(F)[C@H](C7CC7)N6)n5)C4)C3)cc21]

### Preparation of tert-butyl 2-(3-(2,6-bis(benzyloxy)pyridin-3-yl)-1-methyl-1H-indazol-6-yl)-2,6-diazaspiro[3.5]nonane-6-carboxylate

To a stirred solution of 3-(2,6-bis(benzyloxy)pyridin-3-yl)-6-bromo-1-methyl-1H-indazole (1.0 g, 1.99 mmol) and tert-butyl 2,6-diazaspiro[3.5]nonane-6-carboxylate (0.68 g, 0.99 mmol) in 1,4-dioxane (20.0 mL) was added cesium carbonate (1.95 g, 5.99 mmol). The reaction was sparged with argon for 10 minutes. RuPhos (0.09 g, 0.20 mmol) and RuPhos Pd G3 (0.08 g, 0.10 mmol) were added, and sparged for an additional 5 minutes. The reaction mixture was then stirred at 80° C. for 3 hours. The reaction mixture was cooled to room temperature and filtered through celite. The filtrate was concentrated to provide a crude product. The crude product was purified by flash column chromatography (35% ethyl acetate in petroleum ether) to afford tert-butyl 2-(3-(2,6-bis(benzyloxy)pyridin-3-yl)-1-methyl-1H-indazol-6-yl)-2,6-diazaspiro[3.5]nonane-6-carboxylate (1.20 g) as an off-white solid. LC-MS (ESI): m/z=646.68 [M+H]⁺.

### Preparation of tert-butyl 2-(3-(2,6-dioxopiperidin-3-yl)-1-methyl-1H-indazol-6-yl)-2,6-diazaspiro[3.5]nonane-6-carboxylate

To a stirred solution of tert-butyl 2-(3-(2,6-bis(benzyloxy)pyridin-3-yl)-1-methyl-1H-indazol-6-yl)-2,6-diazaspiro[3.5]nonane-6-carboxylate (1.0 g, 1.55 mmol) in THF (40 mL) was added 20% Pd(OH)₂ on carbon (1.0 g) at room temperature. The reaction was stirred under hydrogen (60 psi) atmosphere for 8 hours. The reaction mixture was filtered through celite, the celite was washed with ethyl acetate, and the filtrate was concentrated to afford a crude product. The crude product was washed with n-pentane and dried to afford tert-butyl 2-(3-(2,6-dioxopiperidin-3-yl)-1-methyl-1H-indazol-6-yl)-2,6-diazaspiro[3.5]nonane-6-carboxylate (0.72 g) as a brown solid. LC-MS (ESI): m/z=468.58 [M+H]⁺.

### Preparation of 3-(1-methyl-6-(2,6-diazaspiro[3.5]nonan-2-yl)-1H-indazol-3-yl)piperidine-2,6-dione

To a stirred solution of tert-butyl 2-(3-(2,6-dioxopiperidin-3-yl)-1-methyl-1H-indazol-6-yl)-2,6-diazaspiro[3.5]nonane-6-carboxylate (0.80 g, 1.71 mmol) in DCM (8 mL) at 0° C. was added TFA (8 mL). The reaction mixture was allowed to warm to room temperature over 3 hours. The reaction mixture was concentrated to provide a crude product. The crude product was triturated with diethyl ether (3 times) and the solvent was removed to afford 3-(1-methyl-6-(2,6-diazaspiro[3.5]nonan-2-yl)-1H-indazol-3-yl)piperidine-2,6-dione (0.70 g) as a brown solid. LC-MS (ESI): m/z=368.18 [M+H]⁺.

### Preparation of 3-(6-(6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.5]nonan-2-yl)-1-methyl-1H-indazol-3-yl)piperidine-2,6-dione

To a stirred solution of 3-(1-methyl-6-(2,6-diazaspiro[3.5]nonan-2-yl)-1H-indazol-3-yl)piperidine-2,6-dione (0.40 g, 1.08 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.20 g, 0.43 mmol) in DMSO (8 mL) was added N,N-diisopropylethylamine (1.5 mL, 8.71 mmol). The reaction was stirred at 90° C. for 16 hours. The solvent was removed under vacuum to obtain a crude product. The crude product was purified by prep HPLC to afford 3-(6-(6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.5]nonan-2-yl)-1-methyl-1H-indazol-3-yl)piperidine-2,6-dione (120 mg) as an off-white solid. 1H NMR; 400 MHz, DMSO-d6: δ 10.82 (s, 1H), 8.21 (s, 1H), 8.23 (s, 1H), 8.01-7.92 (m, 2H), 7.68 (d, J=8.40 Hz, 1H), 7.49 (d, J=8.80 Hz, 1H), 6.42-6.35 (m, 2H), 6.15 (br s, 1H), 4.49-4.31 (m, 3H), 3.98-3.89 (m, 4H), 3.64-3.54 (m, 8H), 3.243.21 (m, 2H), 2.67-2.59 (m, 4H), 2.32-2.30 (m, 1H), 2.14-1.99 (m, 1H), 1.99-1.84 (m, 2H), 1.51-1.42 (m, 2H), 0.71-0.69 (m, 1H), 0.52-0.49 (m, 2H), 0.33-0.32 (m, 1H). LC-MS (ESI): m/z=799.49 [M+H]⁺.

### Example 16. 3-(7-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidine-4-carbonyl)piperazin-1-yl)-1-methyl-1H-indazol-3-yl)piperidine-2,6-dione (Compound 397a)

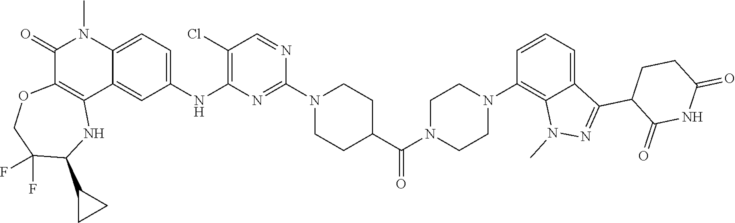 [SMILES: Cn1nc(C2CCC(=O)NC2=O)c2cccc(N3CCN(C(=O)C4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-(4-(3-(2,6-dioxopiperidin-3-yl)-1-methyl-1H-indazol-7-yl)piperazine-1-carbonyl)piperidine-1-carboxylate

In a vial equipped with a stir bar, a mixture of 1-(tert-butoxycarbonyl)piperidine-4-carboxylic acid (315 mg, 1.38 mmol), DIPEA (653 μL, 3.75 mmol), and HATU (570 mg, 1.50 mmol) in DMF (6 mL) was treated with 3-(1-methyl-7-(piperazin-1-yl)-1H-indazol-3-yl)piperidine-2,6-dione hydrochloride (455 mg, 1.25 mmol). The reaction mixture was stirred at 45° C. in a heating block overnight. The reaction mixture was removed from heat and diluted with water and aqueous NH₄Cl. The organics were extracted three times with ethyl acetate, washed once with saturated aqueous NH₄Cl, dried over sodium sulfate, filtered, and concentrated under reduced pressure to provide crude tert-butyl 4-(4-(3-(2,6-dioxopiperidin-3-yl)-1-methyl-1H-indazol-7-yl)piperazine-1-carbonyl)piperidine-1-carboxylate (1.265 g) as a brown oil, which was carried forward without further purification. LC-MS (ESI): m/z=539.6 [M+H]⁺.

### Preparation of 3-(1-methyl-7-(4-(piperidine-4-carbonyl)piperazin-1-yl)-1H-indazol-3-yl)piperidine-2,6-dione

A solution of crude tert-butyl 4-(4-(3-(2,6-dioxopiperidin-3-yl)-1-methyl-1H-indazol-7-yl)piperazine-1-carbonyl)piperidine-1-carboxylate (1.265 g, 1.25 mmol) in dioxane (3.39 mL) was treated with hydrochloric acid (3.12 mL, 12.5 mmol, 4 M in dioxane) and stirred at room temperature for 3 hours. The reaction mixture was concentrated under reduced pressure. The residue was taken up in DMSO/water, filtered through a 0.45 μm hydrophilic PTFE filter, and purified by reverse-phase HPLC using a gradient of 10-60% MeCN (with 0.1% formic acid) in water (with 0.1% formic acid). Fractions containing product were evaporated to give 3-(1-methyl-7-(4-(piperidine-4-carbonyl)piperazin-1-yl)-1H-indazol-3-yl)piperidine-2,6-dione, formic acid (352.9 mg, 58%) as a tan semi-solid. LC-MS (ESI): m/z=439.4 [M+H]⁺.

### Preparation of 3-(7-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidine-4-carbonyl)piperazin-1-yl)-1-methyl-1H-indazol-3-yl)piperidine-2,6-dione

In a vial equipped with a stir bar, a solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (140 mg, 0.300 mmol), 3-(1-methyl-7-(4-(piperidine-4-carbonyl)piperazin-1-yl)-1H-indazol-3-yl)piperidine-2,6-dione, formic acid (145 mg, 0.300 mmol), and DIPEA (261 μL, 1.50 mmol) in DMSO (3 mL) was stirred at 80° C. in a heated reaction block overnight. The reaction mixture was removed from heat and filtered through a 0.45 μm hydrophilic PTFE filter. The material was purified by prep HPLC to afford 3-(7-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidine-4-carbonyl)piperazin-1-yl)-1-methyl-1H-indazol-3-yl)piperidine-2,6-dione (145.4 mg) as a pink solid. 1H NMR (400 MHz, DMSO-d₆) δ 10.88 (s, 1H), 8.84 (s, 1H), 8.20 (d, J=2.3 Hz, 1H), 8.05 (s, 1H), 7.71 (dd, J=9.0, 2.1 Hz, 1H), 7.48-7.38 (m, 2H), 7.08-6.99 (m, 2H), 6.20 (d, J=4.2 Hz, 1H), 4.50-4.29 (m, 6H), 4.27 (s, 3H), 3.56 (s, 3H), 3.28-3.15 (m, OH), 2.92 (dt, J=25.6, 12.0 Hz, 3H), 2.79 (s, 1H), 2.74-2.52 (m, 2H), 2.40-2.27 (m, 1H), 2.17 (dq, J=13.2, 5.4 Hz, 1H), 1.66 (d, J=11.3 Hz, 2H), 1.32 (s, 1H), 0.70 (q, J=6.0 Hz, 1H), 0.55-0.48 (m, 2H), 0.35 (q, J=5.8 Hz, 1H). LC-MS (ESI): m/z=870.9 [M+H]⁺.

### Example 17: 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[4.5]decan-8-yl)amino)-1-methyl-1H-indazol-3-yl)piperidine-2,6-dione (Compound 409a)

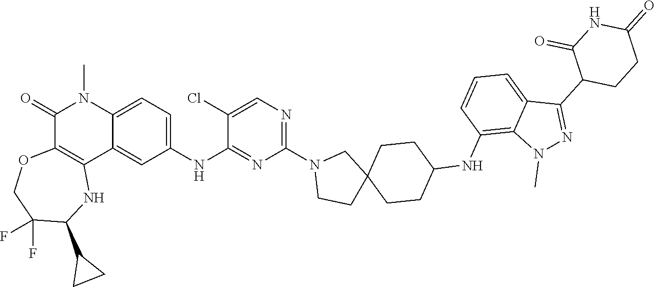 [SMILES: Cn1nc(C2CCC(=O)NC2=O)c2cccc(NC3CCC4(CC3)CCN(c3ncc(Cl)c(Nc5ccc6c(c5)c5c(c(=O)n6C)OCC(F)(F)[C@H](C6CC6)N5)n3)C4)c21]

### Preparation of tert-butyl 8-((3-(2,6-bis(benzyloxy)pyridin-3-yl)-1-methyl-1H-indazol-7-yl) amino)-2-azaspiro [4.5]decane-2-carboxylate

To a stirred solution of 3-(2,6-bis(benzyloxy)pyridin-3-yl)-7-bromo-1-methyl-1H-indazole (0.80 g, 1.60 mmol) in dioxane (10 mL) was added tert-butyl 8-amino-2-azaspiro[4.5]decane-2-carboxylate (0.61 mg, 2.40 mmol) and cesium carbonate (1.56 g, 4.80 mmol). The reaction was degassed with argon for 5 minutes, Pd-PEPPSI-iHeptCl (0.08 g, 0.08 mmol) was added, and the reaction was heated to 80° C. for 16 hours. The reaction mixture was filtered through celite, and the celite was washed with ethyl acetate (100 mL). The collected filtrate was concentrated under vacuum to obtain a crude product. The crude product was purified by column chromatography (27% ethyl acetate in petroleum ether) to afford tert-butyl 8-((3-(2,6-bis(benzyloxy)pyridin-3-yl)-1-methyl-1H-indazol-7-yl)amino)-2-azaspiro[4.5]decane-2-carboxylate[4.5]decane-2-carboxylate (0.81 g) as a pale yellow semi-solid. LC-MS (ESI): m/z=674.62 [M+H]⁺.

### Preparation of tert-butyl 8-((3-(2,6-dioxopiperidin-3-yl)-1-methyl-1H-indazol-7-yl) amino)-2-azaspiro [4.5]decane-2-carboxylate

To a stirred solution of tert-butyl 8-((3-(2,6-bis(benzyloxy)pyridin-3-yl)-1-methyl-1H-indazol-7-yl)amino)-2-azaspiro[4.5]decane-2-carboxylate (0.80 g, 1.19 mmol) in THF (104.0 mL) was added 20% Pd(OH)₂ on carbon (0.80 g, 200% w/w). The reaction was put under H₂ atmosphere (80 psi) and stirred for 16 hours. The reaction mixture was filtered through celite and the celite was washed with 20% THF in DCM (300 mL). The collected filtrate was concentrated under vacuum to obtain a crude product. The crude was washed with n-pentane (20 mL) and dried under vacuum to afford tert-butyl 8-((3-(2,6-dioxopiperidin-3-yl)-1-methyl-1H-indazol-7-yl) amino)-2-azaspiro[4.5]decane-2-carboxylate (0.71 g) as a brown semi-solid. LC-MS (ESI): m/z=496.08 [M+H]⁺.

### Preparation of 3-(7-((2-azaspiro[4.5]decan-8-yl)amino)-1-methyl-1H-indazol-3-yl) piperidine-2,6-dione

To a stirred solution of tert-butyl 8-((3-(2,6-dioxopiperidin-3-yl)-1-methyl-1H-indazol-7-yl) amino)-2-azaspiro[4.5]decane-2-carboxylate (0.70 g, 1.41 mmol) in DCM (7 mL), trifluoro acetic acid (5.0 mL) was added at 0° C. under nitrogen atmosphere. The reaction was stirred at room temperature for 2 hours. Solvent was removed and the crude product was washed with n-pentane (20 mL) and dried under vacuum to afford 3-(7-((2-azaspiro[4.5]decan-8-yl) amino)-n-methyl-11-indazol-3-yl) piperidine-2,6-dione (0.80 g) as a brown semi-solid. LC-MS (ESI): m/z=396.52 [M+H]⁺.

### Preparation of 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[4.5]decan-8-yl)amino)-1-methyl-1H-indazol-3-yl)piperidine-2,6-dione

To a stirred solution of 3-(7-((2-azaspiro[4.5]decan-8-yl)amino)-1-methyl-1H-indazol-3-yl) piperidine-2,6-dione (0.40 g, 1.01 mmol) in DMSO (4 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.24 g, 0.50 mmol) and DIPEA (3.58 mL, 20.22 mmol). The reaction was heated to 100° C. for 8 hours. Solvent was removed under vacuum to obtain a crude product. The crude product was purified by prep HPLC to afford 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[4.5]decan-8-yl)amino)-1-methyl-1H-indazol-3-yl)piperidine-2,6-dione (76.0 mg) as an off-white solid. ¹H NMR, 400 MHz, DMSO-d₆: δ 10.98 (s, 1H), 8.69 (s, 1H), 8.52 (br s, 1H), 8.28 (s, 1H), 8.03 (s, 1H), 7.87-7.82 (m, 1H), 7.43 (d, J=9.20 Hz, 1H), 6.98-6.94 (m, 1H), 6.90-6.86 (m, 1H), 6.52 (d, J=7.20 Hz, 1H), 6.17 (br s, 1H), 4.94-4.93 (m, 1H), 4.45-4.35 (m, 2H), 4.26 (s, 4H), 3.56 (s, 3H), 3.51-3.43 (m, 3H), 3.32-3.22 (m, 3H), 2.63-2.61 (m, 2H), 2.31-2.24 (m, 1H), 1.99-1.89 (m, 3H), 1.72-1.64 (m, 3H), 1.49-1.48 (m, 3H), 1.39-1.34 (m, 1H), 0.82-0.72 (m, 2H), 0.62-0.51 (m, 2H), 0.35-0.31 (m, 1H). LC-MS (ESI): m/z=825.41 [M+H]⁺.

### Example 18: 3-(7-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl) piperidine-2,6-dione (Compound 410a)

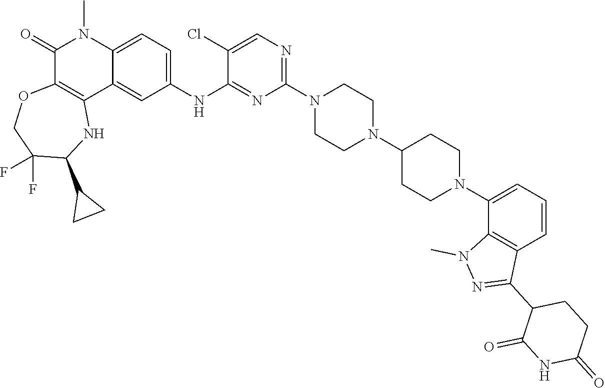 [SMILES: Cn1nc(C2CCC(=O)NC2=O)c2cccc(N3CCC(N4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-(1-(3-(2,6-bis(benzyloxy)pyridin-3-yl)-1-methyl-1H-indazol-7-yl)piperidin-4-yl)piperazine-1-carboxylate

To a stirred solution 3-(2,6-bis(benzyloxy)pyridin-3-yl)-7-bromo-1-methyl-1H-indazole (0.50 g, 1.0 mmol) of in 1,4-dioxane (10 mL, 25 V) was added tert-butyl 4-(piperidin-4-yl)piperazine-1-carboxylate (0.33 g, 1.25 mmol) and sodium tert-butoxide (0.30 g, 3.12 mmol). The reaction was sparged with argon for 5 minutes, Pd-PEPPSI-iHeptCl (0.06 g, 0.06 mmol) was added, and the reaction was sparged with argon for 5 minutes. The reaction was heated to 100° C. for 6 hours. The reaction mixture was filter through celite and concentrated to get crude product. The crude product was purified by flash column chromatography (25% ethyl acetate in petroleum ether) to afford tert-butyl 4-(1-(3-(2,6-bis(benzyloxy)pyridin-3-yl)-1-methyl-1H-indazol-7-yl)piperidin-4-yl)piperazine-1-carboxylate (0.420 g) as a pale yellow semi-solid. LC-MS (ESI): m/z=689.67 [M+H]⁺.

### Preparation of tert-butyl 4-(1-(3-(2,6-dioxopiperidin-3-yl)-1-methyl-1H-indazol-7-yl)piperidin-4-yl)piperazine-1-carboxylate

To a stirred solution of tert-butyl 4-(1-(3-(2,6-bis(benzyloxy)pyridin-3-yl)-1-methyl-1H-indazol-7-yl)piperidin-4-yl)piperazine-1-carboxylate (0.40 g, 0.58 mmol) in THF (52 mL), 20% Pd(OH)₂ on carbon (0.40 g, 4.10 mmol) was added portion wise to the reaction at room temperature. The resultant reaction mixture was put under H₂ atmosphere (50 psi) and stirred at room temperature for 16 hours. The reaction mixture was filter through celite and concentrated to obtain a crude product. The crude product was washed with n-pentane and dried to afford tert-butyl 4-(1-(3-(2,6-dioxopiperidin-3-yl)-1-methyl-1H-indazol-7-yl)piperidin-4-yl)piperazine-1-carboxylate (0.24 g) as brown semi-solid. LC-MS (ESI): m/z=511.59 [M+H]⁺.

### Preparation of 3-(1-methyl-7-(4-(piperazin-1-yl) piperidin-1-yl)-1H-indazol-3-yl) piperidine-2,6-dione

To a stirred solution of tert-butyl 4-(1-(3-(2,6-dioxopiperidin-3-yl)-1-methyl-1H-indazol-7-yl)piperidin-4-yl)piperazine-1-carboxylate (0.30 g, 0.58 mmol) in DCM (3 mL), 4M HCl in 1,4-dioxane (2.15 mL) was added drop-wise to the reaction at 0° C. under nitrogen atmosphere. The reaction was stirred at room temperature for 2 hours. The solvent was removed and the crude product was washed with n-pentane to afford 3-(1-methyl-7-(4-(piperazin-1-yl)piperidin-1-yl)-1H-indazol-3-yl) piperidine-2,6-dione (0.22 g) as a brown semi-solid. LC-MS (ESI): m/z=411.48 [M+H]⁺.

### Preparation of 3-(7-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione

To a stirred solution of 3-(1-methyl-7-(4-(piperazin-1-yl)piperidin-1-yl)-1H-indazol-3-yl)piperidine-2,6-dione (0.10 g, 0.24 mmol) in DMSO (4 mL), (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.057 g, 0.12 mmol) and DIPEA (0.15 mL, 1.22 mmol) were added. The reaction was stirred at 100° C. for 8 hours. Solvent was removed under vacuum to provide a crude product. The crude product was purified by prep HPLC to afford 3-(7-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione (0.048 g) as an off-white solid. ¹H NMR (400 MHz, DMSO-d₆): δ 10.87 (s, 1H), 8.85 (s, 1H), 8.29 (s, 1H), 8.24 (s, 1H), 8.04 (s, 1H), 7.71-7.68 (m, 1H), 7.45-7.42 (m, 1H), 7.36 (m, 1H), 6.99 (d, 2H), 6.23 (s, 1H), 4.44-4.34 (m, 3H), 4.29 (s, 1H), 3.55 (s, 7H), 3.25 (s, 3H), 2.70-2.55 (m, 9H), 2.49 (s, 1H), 2.30 (m, 1H), 2.17 (d, 1H), 1.87 (m, 3H), 1.70 (m, 2H), δ 1.32 (m, 2H), 0.70 (m, 1H), 0.53 (m, 2H), 0.35 (m, 1H). LC-MS (ESI): m/z=841.43 [M+H]⁺.

### Example 19. 3-(7-(3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)azetidin-1-yl)-1-methyl-1H-indazol-3-yl)piperidine-2,6-dione (Compound 422a)

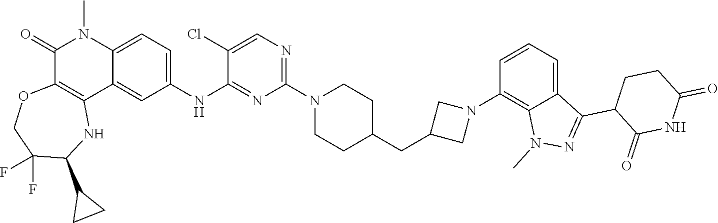 [SMILES: Cn1nc(C2CCC(=O)NC2=O)c2cccc(N3CC(CC4CCN(c5ncc(Cl)c(Nc6ccc7c(c6)c6c(c(=O)n7C)OCC(F)(F)[C@H](C7CC7)N6)n5)CC4)C3)c21]

### Preparation of tert-butyl 4-((1-(3-(2,6-bis(benzyloxy)pyridin-3-yl)-1-methyl-1H-indazol-7-yl) azetidin-3-yl)methyl)piperidine-1-carboxylate

To a stirred solution 3-(2,6-bis(benzyloxy)pyridin-3-yl)-7-bromo-1-methyl-1H-indazole (0.30 g, 0.60 mmol) in dioxane (5.0 mL) was added tert-butyl 4-(azetidin-3-ylmethyl)piperidine-1-carboxylate hydrochloride (0.22 g, 0.77 mmol) and cesium carbonate (0.58 g, 1.79 mmol). The reaction was degassed with argon for 5 minutes, Pd-PEPPSI-iHeptCl (0.03 g, 0.03 mmol) was added, and the reaction was heated at 100° C. for 16 hours. The reaction mixture was filtered through celite and washed with excess of ethyl acetate (200 mL). The collected filtrate was concentrated under vacuum to obtain a crude product. The crude product was purified by column chromatography (28% ethyl acetate in petroleum ether) to afford tert-butyl 4-((1-(3-(2,6-bis(benzyloxy)pyridin-3-yl)-1-methyl-1H-indazol-7-yl)azetidin-3-yl) methyl)piperidine-1-carboxylate (0.38 g) as a pale brown semi-solid. LC-MS (ESI): m/z=674.40 [M+H]⁺.

### Preparation of tert-butyl 4-((1-(3-(2,6-dioxopiperidin-3-yl)-1-methyl-1H-indazol-7-yl)azetidin-3-yl)methyl)piperidine-1-carboxylate

To a stirred solution of tert-butyl 4-((1-(3-(2,6-bis(benzyloxy)pyridin-3-yl)-1-methyl-1H-indazol-7-yl) azetidin-3-yl) methyl)piperidine-1-carboxylate (0.38 g, 0.56 mmol) in THF (100 mL) was added 20% Pd(OH)₂ on carbon (0.38 g, 100% w/w). The reaction was stirred under H₂ atmosphere (80 psi) at room temperature for 16 hours. The reaction mixture was diluted with DCM (100 mL) and filtered through celite. The celite was washed with 30% THF:DCM (300 mL). The filtrate was concentrated under vacuum to obtain tert-butyl 4-((1-(3-(2,6-dioxopiperidin-3-yl)-1-methyl-1H-indazol-7-yl)azetidin-3-yl)methyl)piperidine-1-carboxylate (0.36 g) as a pale brown semi-solid.

LC-MS (ESI): m/z=496.65 [M+H]⁺.

### Preparation of 3-(1-methyl-7-(3-(piperidin-4-ylmethyl)azetidin-1-yl)-1H-indazol-3-yl) piperidine-2,6-dione

To a stirred solution tert-butyl 4-((1-(3-(2,6-dioxopiperidin-3-yl)-1-methyl-1H-indazol-7-yl) azetidin-3-yl)methyl)piperidine-1-carboxylate (0.36 g, 0.73 mmol) in DCM (6.0 mL) was added trifluoroacetic acid (3.1 mL) at 0° C. The reaction was stirred at room temperature for 2 hours. The reaction mixture was concentrated under reduced pressure to obtain crude product. The crude product was washed with n-pentane (20 mL) and dried under reduced pressure to obtain 3-(1-methyl-7-(3-(piperidin-4-ylmethyl)azetidin-1-yl)-1H-indazol-3-yl)piperidine-2,6-dione (0.40 g) as a brown semi-solid. LC-MS (ESI): m/z=398.50 [M+H]⁺.

### Preparation of 3-(7-(3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)azetidin-1-yl)-1-methyl-1H-indazol-3-yl)piperidine-2,6-dione

To a stirred solution of 3-(1-methyl-7-(3-(piperidin-4-ylmethyl)azetidin-1-yl)-1H-indazol-3-yl) piperidine-2,6-dione (0.40 g, 1.01 mmol) in DMSO (4.0 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.24 g, 0.50 mmol) and DIPEA (3.6 mL, 20.23 mmol).

The reaction was heated at 100° C. for 18 hours. The reaction mixture was poured into ice cold water (100 mL). The precipitated solid was filtered and dried to obtain a crude product. The crude product was purified by prep HPLC to afford 3-(7-(3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)azetidin-1-yl)-1-methyl-1H-indazol-3-yl)piperidine-2,6-dione (0.055 mg) as a brown solid. ¹H NMR 400 MHz, DMSO-d₆: δ 10.90 (s, 1H), 8.81 (s, 1H), 8.19 (d, J=1.60 Hz, 1H), 8.02 (s, 1H), 7.72 (dd, J=2.00, 9.20 Hz, 1H), 7.43 (d, J=8.80 Hz, 1H), 7.21 (d, J=8.00 Hz, 1H), 6.99-6.95 (m, 1H), 6.70 (d, J=7.20 Hz, 1H), 6.21 (br s, 1H), 4.44-4.28 (m, 5H), 4.16 (s, 3H), 3.99-3.98 (m, 2H), 3.56 (s, 3H), 3.48-3.43 (m, 2H), 2.77-2.71 (m, 2H), 2.67-2.62 (m, 2H), 2.31-2.29 (m, 1H), 2.21-2.16 (m, 1H), 1.65-1.62 (m, 3H), 1.55-1.53 (m, 3H), 1.41-1.38 (m, 1H), 1.07-1.04 (m, 2H), 0.79-0.69 (m, 1H), 0.52-0.50 (m, 2H), 0.35-0.34 (m, 1H). LC-MS (ESI): m/z=827.51 [M+H]⁺.

### Example 20. 3-(7-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-oxa-7,9-diazabicyclo[3.3.1]nonan-7-yl)methyl)piperidin-1-yl)-1-methyl-1H-indazol-3-yl)piperidine-2,6-dione (Compound 419a)

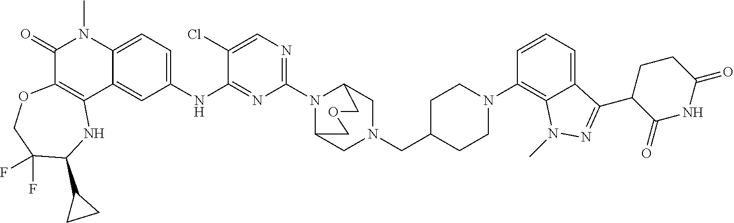 [SMILES: Cn1nc(C2CCC(=O)NC2=O)c2cccc(N3CCC(CN4CC5COCC(C4)N5c4ncc(Cl)c(Nc5ccc6c(c5)c5c(c(=O)n6C)OCC(F)(F)[C@H](C6CC6)N5)n4)CC3)c21]

### Preparation of tert-butyl 7-((1-((benzyloxy)carbonyl)piperidin-4-yl)methyl)-3-oxa-7,9-diazabicyclo[3.3.1]nonane-9-carboxylate

A solution of tert-butyl 3-oxa-7,9-diazabicyclo[3.3.1]nonane-9-carboxylate (600 mg, 2.63 mmol) and benzyl 4-formylpiperidine-1-carboxylate (650 mg, 2.63 mmol) in THF (16 mL) was treated with acetic acid (150 μL, 2.63 mmol), followed by sodium triacetoxyborohydride (836 mg, 3.94 mmol). The reaction mixture was stirred at room temperature for 3 days. The reaction mixture was diluted with water and extracted 3X with ethyl acetate. The combined organics were washed with saturated aqueous NaHCO₃, dried over sodium sulfate, filtered, and concentrated under reduced pressure. The residue was purified by flash column chromatography using a gradient of 20-100% ethyl acetate in heptanes. Fractions containing product were evaporated to give tert-butyl 7-((1-((benzyloxy)carbonyl)piperidin-4-yl)methyl)-3-oxa-7,9-diazabicyclo[3.3.1]nonane-9-carboxylate (1.009 g) as a colorless oil. LC-MS (ESI): m/z=460.2 [M+H]⁺.

### Preparation of tert-butyl 7-(piperidin-4-ylmethyl)-3-oxa-7,9-diazabicyclo[3.3.1]nonane-9-carboxylate

A solution of tert-butyl 7-((1-((benzyloxy)carbonyl)piperidin-4-yl)methyl)-3-oxa-7,9-diazabicyclo[3.3.1]nonane-9-carboxylate (1.01 g, 2.2 mmol) in ethyl acetate (10 mL) was treated with Pd/C (233.6 mg, 10 wt %, 219.5 μmol). The reaction mixture was stirred at room temperature under hydrogen atmosphere (balloon) for 3 days. The reaction mixture was filtered through celite and rinsed with ethyl acetate. The filtrate was concentrated under reduced pressure to give crude tert-butyl 7-(piperidin-4-ylmethyl)-3-oxa-7,9-diazabicyclo[3.3.1]nonane-9-carboxylate (744.2 mg) as a brown oil. LC-MS (ESI): m/z=326.3 [M+H]⁺.

### Preparation of tert-butyl 7-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-9-carboxylate

In a 100 mL flask equipped with stir bar, nitrogen gas was bubbled through a solution of crude tert-butyl 7-(piperidin-4-ylmethyl)-3-oxa-7,9-diazabicyclo[3.3.1]nonane-9-carboxylate (360 mg, 1.11 mmol), 3-(2,6-bis(benzyloxy)pyridin-3-yl)-7-bromo-1-methyl-1H-indazole (664 mg, 1.33 mmol), and RuPhos-Pd G3 (92.5 mg, 111 μmol) in THF (8 mL). To this was added a solution of potassium tert-butoxide (3.32 mL, 3.32 mmol, 1.0 M in THF). The reaction mixture was equipped with a condenser and stirred under nitrogen atmosphere at 70° C. for 3.5 hours. The reaction mixture was quenched with formic acid (167 μL, 4.42 mmol), then was concentrated under reduced pressure. The reaction mixture was taken up in DCM and adsorbed onto celite. The residue was purified by reverse phase column chromatography using a gradient of 10-100% MeCN (w/0.1% formic acid) in water (w/0.1% formic acid). Fractions containing product were concentrated under reduced pressure to afford tert-butyl 7-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-9-carboxylate, formic acid (422 mg) as an orange oil. LC-MS (ESI): m/z=745.3 [M+H]⁺.

### Preparation of tert-butyl 7-((1-(3-(2,6-dioxopiperidin-3-yl)-1-methyl-1H-indazol-7-yl)piperidin-4-yl)methyl)-3-oxa-7,9-diazabicyclo[3.3.1]nonane-9-carboxylate

A solution of tert-butyl 7-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-9-carboxylate (422 mg, 0.566 mmol) in isopropanol (5 mL)/DMF (1 mL)/ethyl acetate (1 mL) was treated with palladium hydroxide on carbon (99.4 mg, 20 wt %, 142 μmol). Hydrogen gas was bubbled through the reaction mixture with a long needle for several minutes, and the reaction mixture was then stirred under hydrogen atmosphere (balloon) at 50° C. in a heating block overnight. The reaction mixture was filtered through celite and rinsed with ethyl acetate and methanol. The filtrate was concentrated under reduced pressure. The reaction mixture was taken up in methanol, filtered through a 0.45 μm hydrophilic PTFE filter, and purified by prep HPLC. Fractions containing the desired product were evaporated and lyophilized to give tert-butyl 7-((1-(3-(2,6-dioxopiperidin-3-yl)-1-methyl-1H-indazol-7-yl)piperidin-4-yl)methyl)-3-oxa-7,9-diazabicyclo[3.3.1]nonane-9-carboxylate (102.6 mg) as a grey solid. LC-MS (ESI): m/z=567.6 [M+H]⁺.

### Preparation of 3-(7-(4-((3-oxa-7,9-diazabicyclo[3.3.1]nonan-7-yl)methyl)piperidin-1-yl)-1-methyl-1H-indazol-3-yl)piperidine-2,6-dione

A solution of tert-butyl 7-((1-(3-(2,6-dioxopiperidin-3-yl)-1-methyl-1H-indazol-7-yl)piperidin-4-yl)methyl)-3-oxa-7,9-diazabicyclo[3.3.1]nonane-9-carboxylate (102.6 mg, 181.0 μmol) in DCM (1.8 mL) was treated with trifluoroacetic acid (279 μL, 3.62 mmol) and stirred at room temperature for 4.5 hours. The reaction mixture was concentrated under reduced pressure, taken up in fresh DCM, and concentrated under reduced pressure again to give 3-(7-(4-((3-oxa-7,9-diazabicyclo[3.3.1]nonan-7-yl)methyl)piperidin-1-yl)-1-methyl-1H-indazol-3-yl)piperidine-2,6-dione, trifluoroacetic acid (105.1 mg) as a tan oil. LC-MS (ESI): m/z=467.4 [M+H]⁺.

### Preparation of 3-(7-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-oxa-7,9-diazabicyclo[3.3.1]nonan-7-yl)methyl)piperidin-1-yl)-1-methyl-1H-indazol-3-yl)piperidine-2,6-dione

In a 2 mL vial equipped with a stir bar, a solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (55.0 mg, 118 μmol), 3-(7-(4-((3-oxa-7,9-diazabicyclo[3.3.1]nonan-7-yl)methyl)piperidin-1-yl)-1-methyl-1H-indazol-3-yl)piperidine-2,6-dione, trifluoroacetic acid (52.5 mg, 90.4 μmol), and DIPEA (78.8 μL, 452 μmol) in DMSO (1 mL) was stirred at 80° C. for 2.5 hours. The reaction mixture was then stirred at 100° C. overnight. The reaction mixture was filtered through a 0.45 μm hydrophilic PTFE filter and purified by prep HPLC to afford 3-(7-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-oxa-7,9-diazabicyclo[3.3.1]nonan-7-yl)methyl)piperidin-1-yl)-1-methyl-1H-indazol-3-yl)piperidine-2,6-dione, formic acid (3.3 mg, 3.9%). LC-MS (ESI): m/z=898.6 [M+H]⁺.

### Example 21: 3-(6-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidine-4-carbonyl)piperazin-1-yl)-1-methyl-1H-indazol-3-yl)piperidine-2,6-dione (Compound 420a)

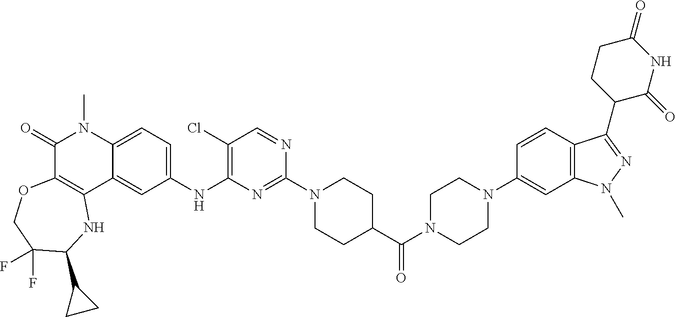 [SMILES: Cn1nc(C2CCC(=O)NC2=O)c2ccc(N3CCN(C(=O)C4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-(4-(3-(2,6-dioxopiperidin-3-yl)-1-methyl-1H-indazol-6-yl)piperazine-1-carbonyl)piperidine-1-carboxylate

A solution of 1-(tert-butoxycarbonyl)piperidine-4-carboxylic acid (126 mg, 550 μmol), DIPEA (261 μL, 1.50 mmol), and HATU (228 mg, 600 μmol) in DMF (2.5 mL) was treated with 3-(1-methyl-6-(piperazin-1-yl)-1H-indazol-3-yl)piperidine-2,6-dione (164 mg, 0.500 mmol). The reaction mixture was stirred at room temperature overnight. The reaction mixture was filtered through a hydrophilic PTFE 0.45 μm syringe filter and rinsed with methanol. The filtrate was purified by prep HPLC. Fractions containing product were evaporated to give tert-butyl 4-(4-(3-(2,6-dioxopiperidin-3-yl)-1-methyl-1H-indazol-6-yl)piperazine-1-carbonyl)piperidine-1-carboxylate (126.7 mg) as a tan foam. LC-MS (ESI): m/z=539.5 [M+H]⁺.

### Preparation of 3-(1-methyl-6-(4-(piperidine-4-carbonyl)piperazin-1-yl)-1H-indazol-3-yl)piperidine-2,6-dione, trifluoroacetic acid

To a solution of tert-butyl 4-(4-(3-(2,6-dioxopiperidin-3-yl)-1-methyl-1H-indazol-6-yl)piperazine-1-carbonyl)piperidine-1-carboxylate (126.7 mg, 235.2 μmol) in DCM (2 mL) was added trifluoroacetic acid (362 μL, 4.70 mmol). The reaction mixture was stirred at room temperature overnight. The reaction mixture was concentrated under reduced pressure. The residue was washed once with DCM (2 mL) and again concentrated under reduced pressure to give 3-(1-methyl-6-(4-(piperidine-4-carbonyl)piperazin-1-yl)-1H-indazol-3-yl)piperidine-2,6-dione, trifluoroacetic acid (130 mg) as a brown oil. LC-MS (ESI): m/z=439.3 [M+H]⁺.

### Preparation of 3-(6-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidine-4-carbonyl)piperazin-1-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (110 mg, 235 μmol), 3-(1-methyl-6-(4-(piperidine-4-carbonyl)piperazin-1-yl)-1H-indazol-3-yl)piperidine-2,6-dione, trifluoroacetic acid (130 mg, 235 μmol), and DIPEA (205 μL, 1.18 mmol) in DMSO (2 mL) was stirred at 80° C. for 8 hours. The reaction mixture was filtered through a 0.45 μm hydrophilic PTFE filter and purified by prep HPLC to afford 3-(6-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidine-4-carbonyl)piperazin-1-yl)-1-methyl-1H-indazol-3-yl)piperidine-2,6-dione (21.7 mg, 11%). 1H NMR (400 MHz, DMSO-d₆) δ 10.85 (s, 1H), 8.80 (s, 1H), 8.25-8.16 (m, 1H), 8.04 (s, 1H), 7.72 (dd, J=9.1, 2.2 Hz, 1H), 7.53 (d, J=8.9 Hz, 1H), 7.43 (d, J=9.1 Hz, 1H), 6.95 (dd, J=9.0, 2.0 Hz, 1H), 6.88 (d, J=1.9 Hz, 1H), 6.19 (s, 1H), 4.52-4.22 (m, 2H), 3.90 (s, 3H), 3.67 (m, 4H), 3.55 (s, 3H), 3.20 (m, 6H), 2.90 (q, J=11.9 Hz, 3H), 2.69-2.56 (m, 2H), 2.38-2.24 (m, 1H), 2.16 (dd, J=13.3, 5.8 Hz, 1H), 1.65 (d, J=12.8 Hz, 2H), 1.51 (t, J=11.5 Hz, 2H), 0.69 (t, J=7.9 Hz, 1H), 0.51 (t, J=6.2 Hz, 2H), 0.34 (q, J=5.7 Hz, 1H). LC-MS (ESI): m/z=870.5 [M+H]⁺.

### Example 22: 3-(7-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)propan-2-yl)piperidin-1-yl)-1-methyl-1H-indazol-3-yl)piperidine-2,6-dione (Compound 421a)

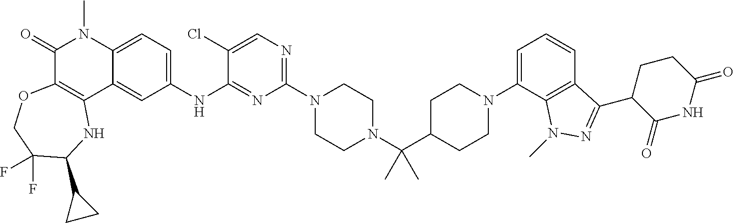 [SMILES: Cn1nc(C2CCC(=O)NC2=O)c2cccc(N3CCC(C(C)(C)N4CCN(c5ncc(Cl)c(Nc6ccc7c(c6)c6c(c(=O)n7C)OCC(F)(F)[C@H](C7CC7)N6)n5)CC4)CC3)c21]

### Preparation of benzyl 3,4-dihydroxypyrrolidine-1-carboxylate

To a stirred solution of benzyl 2,5-dihydro-1H-pyrrole-1-carboxylate (17.6 g, 86.66 mmol) and N-methyl morpholine N-oxide (10.15 g, 86.66 mmol) in THF (264 mL) was added dropwise a solution of osmium tetraoxide (4.40 g, 17.33 mmol) in tert-butanol (106 mL) at 0° C. After stirring for 24 hours, the reaction mixture was quenched with cold water (100 mL) and extracted with ethyl acetate (3×150 mL). The organic layers were separated, dried over anhydrous sodium sulfate, filtered, and concentrated to get crude product. The crude product was purified by flash chromatography (10% methanol in dichloromethane) to afford benzyl 3,4-dihydroxypyrrolidine-1-carboxylate (7.75 g) as a brown gum. LC-MS (ESI): m/z=238.06 [M+H]⁺.

### Preparation of benzyl bis(2-oxoethyl)carbamate

To a stirred solution of benzyl 3,4-dihydroxypyrrolidine-1-carboxylate (7.75 g, 32.68 mmol) in dichloromethane (78 mL) was added sodium periodate (10.48 g, 49.02 mmol) portion wise at 0° C. The resulting reaction mixture was stirred at room temperature for 1 hour. The reaction mixture was quenched with cold water (100 mL) and extracted with ethyl acetate (3×150 mL). The organic layers were separated, dried over anhydrous sodium sulfate, filtered, and concentrated to provide benzyl bis(2-oxoethyl)carbamate (6.1 g) as a brown gum. LC-MS (ESI): m/z=234.20 [M−H]⁻.

### Preparation of tert-butyl 4-(2-(1-((benzyloxy)carbonyl)piperidin-4-yl)propan-2-yl)piperazine-1-carboxylate

To a solution of benzyl bis(2-oxoethyl)carbamate (6.10 g, 25.94 mmol), tert-butyl 4-(2-aminopropan-2-yl)piperidine-1-carboxylate (3.14 g, 12.97 mmol) in methanol (122 mL) was added acetic acid (1.55 mL, 60.05 mmol) at 0° C. The reaction mixture was stirred at room temperature for 2 hours. 2-Methylpyridine borane complex (5.55 g, 51.89 mmol) was added portion wise, and the reaction was stirred at room temperature for 16 hours. The reaction mixture was quenched with ice-cold water (100 mL) and extracted with DCM (3×150 mL). The combined organic layers were washed with brine solution (100 mL), dried over anhydrous sodium sulfate, filtered, and concentrated under vacuum to get crude product. The crude product was purified by prep HPLC to afford tert-butyl 4-(2-(1-((benzyloxy)carbonyl)piperidin-4-yl)propan-2-yl)piperazine-1-carboxylate (0.55 g) as an off white solid. LC-MS (ESI): m/z=446.61 [M+H]⁺.

### Preparation of tert-butyl 4-(2-(piperidin-4-yl)propan-2-yl)piperazine-1-carboxylate

To a stirred solution of tert-butyl 4-(2-(1-((benzyloxy)carbonyl)piperidin-4-yl)propan-2-yl)piperazine-1-carboxylate (0.55 g, 1.12 mmol) in 2-propanol (11 mL) was added 20% Pd(OH)₂ on carbon (0.55 g). The reaction was put under a hydrogen atmosphere (20 psi) and stirred at room temperature for 16 hours. The reaction mixture was diluted with methanol (120 mL) and filtered through celite. The filtrate was concentrated under vacuum to obtain tert-butyl 4-(2-(piperidin-4-yl)propan-2-yl)piperazine-1-carboxylate (0.28 g) as a brown gum. LC-MS (ESI): m/z=312.21 [M+H]⁺.

### Preparation of tert-butyl 4-(2-(1-(3-(2,6-bis(benzyloxy)pyridin-3-yl)-1-methyl-1H-indazol-7-yl)piperidin-4-yl)propan-2-yl)piperazine-1-carboxylate

To a stirred solution of 3-(2,6-bis(benzyloxy)pyridin-3-yl)-7-bromo-1-methyl-1H-indazole (0.22 g, 0.44 mmol) and tert-butyl 4-(2-(piperidin-4-yl)propan-2-yl)piperazine-1-carboxylate (0.28 g, 0.89 mmol) in 1,4-dioxane (4.5 mL) was added cesium carbonate (0.43 g, 1.34 mmol). The reaction was degassed with argon for 10 minutes, Pd-PEPPSI-iHeptCl (0.02 g, 0.02 mmol) was added, and the reaction was heated at 100° C. for 16 hours. The reaction mixture was filtered through celite and concentrated to obtain a crude product. The crude product was purified by flash column chromatography (30% ethyl acetate in petroleum ether) to afford tert-butyl 4-(2-(1-(3-(2,6-bis(benzyloxy)pyridin-3-yl)-1-methyl-1H-indazol-7-yl)piperidin-4-yl)propan-2-yl)piperazine-1-carboxylate (0.18 g) as a brown gum. LC-MS (ESI): m/z=731.90 [M+H]⁺.

### Preparation of tert-butyl 4-(2-(1-(3-(2,6-dioxopiperidin-3-yl)-1-methyl-1H-indazol-7-yl)piperidin-4-yl)propan-2-yl)piperazine-1-carboxylate

To a stirred solution of tert-butyl 4-(2-(1-(3-(2,6-bis(benzyloxy)pyridin-3-yl)-1-methyl-1H-indazol-7-yl)piperidin-4-yl)propan-2-yl)piperazine-1-carboxylate (0.18 g, 0.24 mmol) in THF (3.6 mL) was added 20% Pd(OH)₂ on carbon (0.18 g). The reaction was put under hydrogen atmosphere (20 psi) and stirred at room temperature for 8 hours. The reaction mixture was diluted with DCM (80 mL) and filtered through celite. The filtrate was concentrated under vacuum to afford tert-butyl 4-(2-(1-(3-(2,6-dioxopiperidin-3-yl)-1-methyl-1H-indazol-7-yl)piperidin-4-yl)propan-2-yl)piperazine-1-carboxylate (0.13 g) as a brown solid. LC-MS (ESI): m/z=553.69 [M+H]⁺.

### Preparation of 3-(1-methyl-7-(4-(2-(piperazin-1-yl)propan-2-yl)piperidin-1-yl)-1H-indazol-3-yl)piperidine-2,6-dione

To a stirred solution of tert-butyl 4-(2-(1-(3-(2,6-dioxopiperidin-3-yl)-1-methyl-1H-indazol-7-yl)piperidin-4-yl)propan-2-yl)piperazine-1-carboxylate (0.13 g, 1.31 mmol) in DCM (2.6 mL) was added TFA (0.4 mL). The reaction was stirred at room temperature for 1 hour. The reaction mixture was concentrated and co-distilled with petroleum ether to obtain 3-(1-methyl-7-(4-(2-(piperazin-1-yl)propan-2-yl)piperidin-1-yl)-1H-indazol-3-yl)piperidine-2,6-dione as a brown solid (0.15 g, TFA salt). LC-MS (ESI): m/z=453.58 [M+H]⁺.

### Preparation of 3-(7-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)propan-2-yl)piperidin-1-yl)-1-methyl-1H-indazol-3-yl)piperidine-2,6-dione

To a stirred solution of 3-(1-methyl-7-(4-(2-(piperazin-1-yl)propan-2-yl)piperidin-1-yl)-1H-indazol-3-yl)piperidine-2,6-dione (0.075 g, 0.13 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.038 g, 0.08 mmol) in DMSO (1.5 mL) was added DIPEA (0.23 mL, 1.36 mmol). The reaction was heated to 100° C. and stirred for 3 hours. Solvent was removed under vacuum and the crude product was purified by prep HPLC to afford 3-(7-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)propan-2-yl)piperidin-1-yl)-1-methyl-1H-indazol-3-yl)piperidine-2,6-dione (0.015 g) as an off-white solid. ¹H NMR 400 MHz, DMSO-d₆: δ 10.90 (s, 1H), 8.76 (s, 1H), 8.22 (m, 1H), 8.02 (s, 1H), 7.74-7.72 (m, 1H), 7.46-7.37 (m, 2H), 7.12-7.02 (m, 2H), 6.20 (s, 1H), 4.44 (s, 2H), 4.34-4.35 (m, 3H), 4.25-4.31 (m, 3H), 3.57 (s, 3H), 3.21-2.68 (m, 13H), 2.33-2.25 (m, 2H), 1.77-1.74 (m, 3H), 1.35 (s, 1H), 1.23 (s, 1H), 1.12-1.11 (m, 2H), 0.90 (s, 6H), 0.71-0.69 (m, 1H), 0.52 (t, J=6.00 Hz, 2H), 0.36.34 (m, 1H). LC-MS (ESI): m/z=884.60 [M+H]⁺.

### Example 23: 3-(6-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione (Compound 411a)

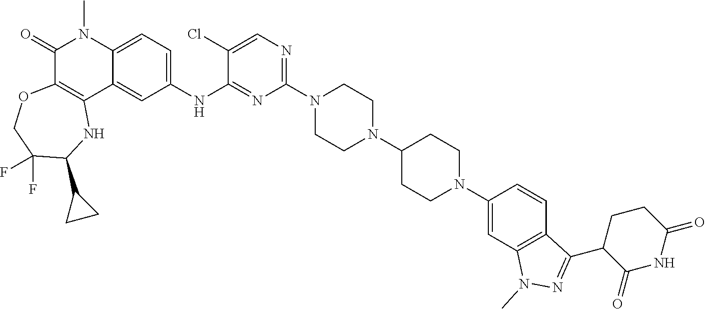 [SMILES: Cn1nc(C2CCC(=O)NC2=O)c2ccc(N3CCC(N4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-(1-(3-(2,6-bis(benzyloxy)pyridin-3-yl)-1-methyl-1H-indazol-6-yl)piperidin-4-yl)piperazine-1-carboxylate

To a stirred solution 3-(2,6-bis(benzyloxy)pyridin-3-yl)-6-bromo-1-methyl-1H-indazole (0.50 g, 1.0 mmol) of in 1,4-dioxane (10 mL) was added tert-butyl 4-(piperidin-4-yl) piperazine-1-carboxylate (0.33 g, 1.25 mmol) and cesium carbonate (1.26 g, 3.75 mmol). The reaction was degassed with argon for 5 minutes, RuPhos (0.006 g, 0.01 mmol) and RuPhos Pd G3 (0.05 g, 0.05 mmol) were added, and the reaction was heated to 100° C. for 6 hours. The reaction mixture was filtered through celite and the filtrate was concentrated to provide a crude product. The crude was purified by flash column chromatography (25% ethyl acetate in petroleum ether) to afford tert-butyl 4-(1-(3-(2,6-bis(benzyloxy)pyridin-3-yl)-1-methyl-1H-indazol-6-yl)piperidin-4-yl)piperazine-1-carboxylate (0.520 g) as a pale yellow semi-solid. LC-MS (ESI): m/z=689.67 [M+H]⁺.

### Preparation of tert-butyl 4-(1-(3-(2,6-dioxopiperidin-3-yl)-1-methyl-1H-indazol-6-yl)piperidin-4-yl)piperazine-1-carboxylate

To a stirred solution of tert-butyl 4-(1-(3-(2,6-bis(benzyloxy)pyridin-3-yl)-1-methyl-1H-indazol-6-yl)piperidin-4-yl)piperazine-1-carboxylate (0.47 g, 0.68 mmol) in THF (61 mL) 20% Pd(OH)₂ on carbon (0.47 g, 6.86 mmol) was added portion wise. The reaction was put under hydrogen atmosphere (50 psi) and stirred at room temperature for 16 hours. The reaction mixture was filtered through celite and the filtrate was concentrated to provide a crude product. The crude product was triturated with n-pentane and dried to afford tert-butyl 4-(1-(3-(2,6-dioxopiperidin-3-yl)-1-methyl-1H-indazol-6-yl)piperidin-4-yl)piperazine-1-carboxylate (0.30 g) as a brown semi-solid. LC-MS (ESI): m/z=511.59 [M+H]⁺.

### Preparation of 3-(1-methyl-6-(4-(piperazin-1-yl) piperidin-1-yl)-1H-indazol-3-yl) piperidine-2, 6-dione

To a stirred solution of tert-butyl 4-(1-(3-(2,6-dioxopiperidin-3-yl)-1-methyl-1H-indazol-6-yl)piperidin-4-yl)piperazine-1-carboxylate (0.30 g, 0.732 mmol) in DCM (3 mL) was added 4M HCl in 1,4-dioxane (2.15 mL) at 0° C. The reaction stirred at room temperature for 2 hours. The reaction was concentrated, and the crude product was washed with n-pentane to afford 3-(1-methyl-6-(4-(piperazin-1-yl) piperidin-1-yl)-1H-indazol-3-yl) piperidine-2, 6-dione (0.22 g) as a brown semi-solid. LC-MS (ESI): m/z=411.48 [M+H]⁺.

### Preparation of 3-(6-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione

To a stirred solution of 3-(1-methyl-6-(4-(piperazin-1-yl) piperidin-1-yl)-1H-indazol-3-yl) piperidine-2, 6-dione (0.10 g, 0.24 mmol) in DMSO (4 mL) was added (S)-2-cyclopropyl-10-((2,5-dichloropyrimidin-4-yl)amino)-3,3-difluoro-7-methyl-1,2,3,4-tetrahydro-[1,4]oxazepino [2,3-c]quinolin-6(7H)-one (0.057 g, 0.12 mmol) and DIPEA (0.15 mL, 1.22 mmol). The reaction was heated to 100° C. for 8 hours. The solvent was removed under vacuum to provide a crude product.

The crude product was purified by prep HPLC to afford 3-(6-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione (59 mg) as an off-white solid. ¹H NMR (400 MHz, DMSO-d₆): δ 10.82 (s, 1H), 8.83 (s, 1H), 8.30 (s, 1H), 8.22 (s, 1H), 8.04 (s, 1H), 7.71-7.68 (m, 1H), 7.45-7.42 (m, 1H), 6.90 (d, 1H), 6.83 (s, 1H), 6.23 (s, 1H), 4.42 (m, J=8.4 Hz, 1H), 4.26-4.22 (m, 1H), 3.87-3.80 (m, 5H), 3.55 (s, 7H), 2.75-2.67 (m, 2H), 2.63-2.59 (m, 6H), 2.38 (s, 1H), 2.32 (m, 1H), 2.17 (d, 1H), 1.84 (m, 2H), 1.54 (m, 2H), δ 1.32 (m, 2H), 0.70 (m, 1H), 0.53 (m, 2H), 0.35 (m, 1H). LC-MS (ESI): m/z=840.43 [M+H]⁺.

### Example 24: 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-1-methyl-1H-indazol-3-yl)piperidine-2,6-dione (Compound 412a)

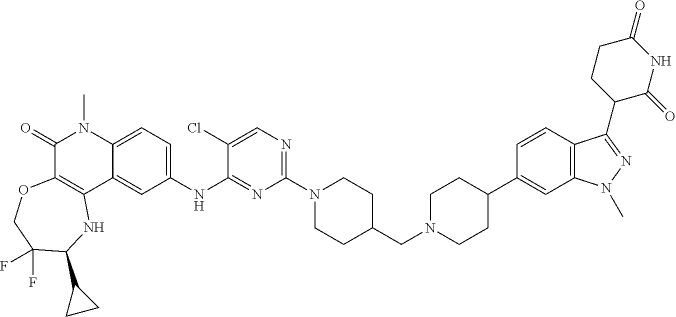 [SMILES: Cn1nc(C2CCC(=O)NC2=O)c2ccc(C3CCN(CC4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-(3-(2,6-bis(benzyloxy)pyridin-3-yl)-1-methyl-1H-indazol-6-yl)-3,6-dihydropyridine-1(2H)-carboxylate

To a stirred solution of 3-(2,6-bis(benzyloxy)pyridin-3-yl)-6-bromo-1-methyl-1H-indazole (2.0 g, 4.0 mmol) in 1,4-dioxane (32.0 mL) and water (8.0 mL) was added tert-butyl 4-(4,4,5,5-tetramethyl-1,3,2-dioxaborolan-2-yl)-3,6-dihydropyridine-1(2H)-carboxylate (1.5 g, 4.8 mmol). The reaction was purged with nitrogen for 15 minutes. Cesium carbonate (3.91 g, 12.01 mmol), Xantphos (0.92 g, 1.6 mmol), and Pd₂(dba)₃ (0.73 g, 0.80 mmol) were added. The reaction was stirred at 100° C. for 16 hours. The reaction was quenched with water and extracted into ethyl acetate. The combined organic layers were washed with brine solution, dried over anhydrous sodium sulfate, filtered, and concentrated under vacuum to provide a crude product. The crude product was purified by flash column chromatography (20% ethyl acetate in petroleum ether) to afford tert-butyl 4-(3-(2,6-bis(benzyloxy)pyridin-3-yl)-1-methyl-1H-indazol-6-yl)-3,6-dihydropyridine-1(2H)-carboxylate (2.0 g) as a yellow solid. LC-MS (ESI): m/z=603.66 [M+H]⁺.

### Preparation of 3-(2,6-bis(benzyloxy)pyridin-3-yl)-1-methyl-6-(1,2,3,6-tetrahydropyridin-4-yl)-1H-indazole hydrochloride

To a stirred solution of tert-butyl 4-(3-(2,6-bis(benzyloxy)pyridin-3-yl)-1-methyl-1H-indazol-6-yl)-3,6-dihydropyridine-1(2H)-carboxylate (2.0 g, 1.35 mmol) in DCM (20.0 mL) was added 4M HCl in 1,4-dioxane (10 mL). The reaction was stirred at room temperature for 2 hours. The reaction solvent was evaporated under vacuum to provide a crude product. The crude product was triturated using diethyl ether to obtain 3-(2,6-bis(benzyloxy)pyridin-3-yl)-1-methyl-6-(1,2,3,6-tetrahydropyridin-4-yl)-1H-indazole hydrochloride (1.5 g). LC-MS (ESI): m/z=503.57 [M+H]⁺.

### Preparation of tert-butyl 4-((4-(3-(2,6-bis(benzyloxy)pyridin-3-yl)-1-methyl-1H-indazol-6-yl)-3,6-dihydropyridin-1(2H)-yl)methyl)piperidine-1-carboxylate

To a stirred solution of 3-(2,6-bis(benzyloxy)pyridin-3-yl)-1-methyl-6-(1,2,3,6-tetrahydropyridin-4-yl)-1H-indazole hydrochloride (1.0 g, 1.85 mmol) and tert-butyl 4-formylpiperidine-1-carboxylate (0.475 g, 2.22 mmol) in THF (20 mL) at 0° C., titanium(IV)isopropoxide was added.

After 1 hour, sodium cyanoborohydride (0.35 g, 5.56 mmol) was added and the reaction mixture was heated at 80° C. for 16 hours. The reaction was quenched with water and extracted into ethyl acetate. The combined organic layers were washed with brine solution, dried over anhydrous sodium sulfate, filtered, and concentrated under vacuum to get crude product. The crude was purified by flash column chromatography (ethyl acetate in petroleum ether) to afford tert-butyl 4-((4-(3-(2,6-bis(benzyloxy)pyridin-3-yl)-1-methyl-1H-indazol-6-yl)-3,6-dihydropyridin-1(2H)-yl)methyl)piperidine-1-carboxylate (0.6 g) as a yellow solid. LC-MS (ESI): m/z=700.69 [M+H]⁺.

### Preparation of tert-butyl 4-((4-(3-(2,6-dioxopiperidin-3-yl)-1-methyl-1H-indazol-6-yl)piperidin-1-yl)methyl)piperidine-1-carboxylate

To a stirred solution of tert-butyl 4-((4-(3-(2,6-bis(benzyloxy)pyridin-3-yl)-1-methyl-1H-indazol-6-yl)-3,6-dihydropyridin-1(2H)-yl)methyl)piperidine-1-carboxylate (0.6 g, 0.85 mmol) in THF (24 mL) was added 20% Pd (OH)₂ on carbon (moisture 50% wet) (1.2 g). The reaction was put under hydrogen atmosphere (balloon pressure) and stirred at room temperature for 24 hours. The reaction mixture was diluted with ethyl acetate and filtered through celite. The celite was washed with 30% THF in ethyl acetate. The filtrate was concentrated under vacuum to provide a crude product. The crude product was triturated with diethyl ether to afford tert-butyl 4-((4-(3-(2,6-dioxopiperidin-3-yl)-1-methyl-1H-indazol-6-yl)piperidin-1-yl)methyl)piperidine-1-carboxylate (0.25 g) as an off white solid. LC-MS (ESI): m/z=524.66 [M+H]⁺.

### Preparation of 3-(1-methyl-6-(1-(piperidin-4-ylmethyl)piperidin-4-yl)-1H-indazol-3-yl)piperidine-2,6-dione hydrochloride

To a solution of tert-butyl 4-((4-(3-(2,6-dioxopiperidin-3-yl)-1-methyl-1H-indazol-6-yl)piperidin-1-yl)methyl)piperidine-1-carboxylate (0.25 g, 0.47 mmol) in DCM (2.5 mL) was added 4M HCl in 1,4-dioxane (1.25 mL). The reaction mixture was stirred at room temperature for 3 hours. The reaction mixture was concentrated under vacuum to afford a crude product. The crude product was triturated using diethyl ether to obtain 3-(1-methyl-6-(1-(piperidin-4-ylmethyl)piperidin-4-yl)-1H-indazol-3-yl)piperidine-2,6-dione hydrochloride (0.15 g). LC-MS (ESI): m/z=424.51 [M+H]⁺.

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-1-methyl-1H-indazol-3-yl)piperidine-2,6-dione

To a stirred solution of 3-(1-methyl-6-(1-(piperidin-4-ylmethyl)piperidin-4-yl)-1H-indazol-3-yl)piperidine-2,6-dione hydrochloride (0.15 g, 0.32 mmol) in DMSO (6.0 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.076 g, 0.16 mmol) and DIPEA (0.3 mL, 1.63 mmol).

The reaction mixture was stirred at 100° C. for 5 hours. Solvent was removed under vacuum to obtain a crude product. The crude product was purified by prep HPLC to afford 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-1-methyl-1H-indazol-3-yl)piperidine-2,6-dione (0.037 g) as an off-white solid. 1H NMR (400 MHz, DMSO-d₆): δ 10.87 (s, 1H), 8.77 (s, 1H), 8.24 (bs, 1H), 8.19-8.18 (d, J=1.6 Hz, 1H), 8.02 (s, 1H), 7.75-7.72 (m, 1H), 7.61-7.58 (d, J=8.4 Hz, 1H), 7.46-7.42 (t, J=7.8 Hz, 2H), 7.03-7.01 (d, J=8.4 Hz, 1H), 6.14 (bs, 1H), 4.46-4.30 (m, 5H), 3.96 (s, 3H), 3.57 (s, 3H), 2.94 (bs, 2H), 2.81-2.75 (t, J=12.2 Hz, 2H), 2.67-2.60 (m, 3H), 2.50-2.49 (m, 1H), 2.18-2.14 (m, 3H), 1.98 (bs, 2H), 1.78-1.72 (m, 7H), 1.35 (bs, 1H), 1.02-1.00 (m, 2H), 0.70 (m, 1H), 0.53-0.50 (t, J=5.8 Hz, 2H), 0.36-0.34 (m, 1H). LC-MS (ESI): m/z=855.51 [M+H]⁺.

### Example 25: 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-1,4-diazepan-1-yl)methyl)piperidin-1-yl)-1-methyl-1H-indazol-3-yl)piperidine-2,6-dione (Compound 413a)

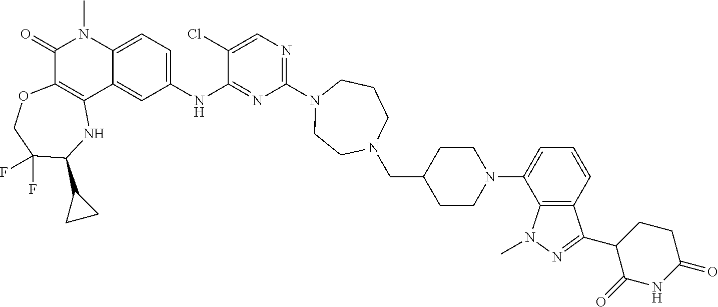 [SMILES: Cn1nc(C2CCC(=O)NC2=O)c2cccc(N3CCC(CN4CCCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-((1-((benzyloxy)carbonyl)piperidin-4-yl)methyl)-1,4-diazepane-1-carboxylate

To a stirred solution of tert-butyl 1,4-diazepane-1-carboxylate (4.00 g, 19.97 mmol) and benzyl 4-formylpiperidine-1-carboxylate (4.93 g, 19.97 mmol) in DCM (40 mL), acetic acid (1.14 mL, 19.97 mmol) was added. After stirring for 1 hour, sodium triacetoxyborohydride (8.46 g, 39.94 mmol) was added, and the reaction was stirred at room temperature for 16 hours. The reaction mixture was quenched with ammonium chloride solution (100 mL) and extracted with DCM (2×100 mL). The combined organic layers were washed with brine solution, dried over anhydrous sodium sulfate, filtered, and concentrated under reduced pressure to afford tert-butyl 4-((1-((benzyloxy)carbonyl)piperidin-4-yl)methyl)-1,4-diazepane-1-carboxylate (5.1 g) as a pale yellow, gummy solid. LC-MS (ESI): m/z=432.32 [M+H]⁺.

### Preparation of tert-butyl 4-(piperidin-4-ylmethyl)-1,4-diazepane-1-carboxylate

To a solution of tert-butyl 4-((1-((benzyloxy)carbonyl)piperidin-4-yl)methyl)-1,4-diazepane-1-carboxylate (3.00 g, 6.95 mmol) in THF (120 mL), 20% Pd(OH)₂ on carbon (1.50 g) was added. The reaction was put under hydrogen atmosphere (80 psi) and stirred at room temperature for 16 hours. The reaction mixture was diluted with THF (50 mL) and filtered through celite. The celite was washed with 200 mL of THF:DCM (1:1) and the filtrate was concentrated to afford tert-butyl 4-(piperidin-4-ylmethyl)-1,4-diazepane-1-carboxylate (1.8 g) as a brown gummy solid. ¹H NMR (400 MHz, CDCl₃): δ 3.48-3.39 (m, 4H), 3.08 (d, J=12.00 Hz, 2H), 2.65-2.56 (m, 6H), 2.28-2.29 (m, 2H), 1.79-1.73 (m, 4H), 1.43 (s, 9H), 1.12-1.03 (m, 2H).

### Preparation of tert-butyl 4-((1-(3-(2,6-bis(benzyloxy)pyridin-3-yl)-1-methyl-1H-indazol-7-yl)piperidin-4-yl)methyl)-1,4-diazepane-1-carboxylate

To a stirred solution of 3-(2,6-bis(benzyloxy)pyridin-3-yl)-7-bromo-1-methyl-1H-indazole (0.50 g, 0.99 mmol) and tert-butyl 4-(piperidin-4-ylmethyl)-1,4-diazepane-1-carboxylate (0.89 g, 2.99 mmol) in 1,4-dioxane (10 mL), cesium carbonate (0.97 g, 2.99 mmol) was added. The reaction was degassed with argon for 15 minutes. Pd-PEPPSI-IHeptCl (0.048 g, 0.05 mmol) was added, and the reaction was stirred at 100° C. for 16 hours. The reaction mixture was diluted with ethyl acetate (40 mL) and filtered through celite. The celite was washed with 10% MeOH:DCM (250 mL) and the filtrate was concentrated to obtain a crude product. The crude product was purified by flash chromatography (10-15% ethyl acetate in petroleum ether) to afford tert-butyl 4-((1-(3-(2,6-bis(benzyloxy)pyridin-3-yl)-1-methyl-1H-indazol-7-yl)piperidin-4-yl)methyl)-1,4-diazepane-1-carboxylate (0.33 g) as a pale yellow solid. LC-MS (ESI): m/z=717.71 [M+H]⁺.

### Preparation of tert-butyl 4-((1-(3-(2,6-dioxopiperidin-3-yl)-1-methyl-1H-indazol-7-yl)piperidin-4-yl)methyl)-1,4-diazepane-1-carboxylate

To a solution of tert-butyl 4-((1-(3-(2,6-bis(benzyloxy)pyridin-3-yl)-1-methyl-1H-indazol-7-yl)piperidin-4-yl)methyl)-1,4-diazepane-1-carboxylate (0.33 g, 0.46 mmol) in THF (32 mL) was added 20% Pd(OH)₂ on carbon (0.66 g). The reaction was put under hydrogen atmosphere (80 psi) and stirred at room temperature for 5 hours. The reaction mixture was diluted with THF (60 mL) and filtered through celite. The celite was washed with THF:DCM (1:1, 200 mL) and the filtrate was concentrated to afford tert-butyl 4-((1-(3-(2,6-dioxopiperidin-3-yl)-1-methyl-1H-indazol-7-yl)piperidin-4-yl)methyl)-1,4-diazepane-1-carboxylate (0.22 g) as a brown solid. LC-MS (ESI): m/z=551.68 [M+H]⁺.

### Preparation of 3-(7-(4-((1,4-diazepan-1-yl)methyl)piperidin-1-yl)-1-methyl-1H-indazol-3-yl)piperidine-2,6-dione

To a stirred solution of tert-butyl 4-((1-(3-(2,6-dioxopiperidin-3-yl)-1-methyl-1H-indazol-7-yl)piperidin-4-yl)methyl)-1,4-diazepane-1-carboxylate (0.59 g, 1.07 mmol) in DCM (6 mL), TFA (3.0 mL) was added at 0° C. The reaction was allowed to warm to room temperature over 3 hours with stirring. The reaction mixture was concentrated under reduced pressure and co-distilled with DCM (3×10 mL) to afford 3-(7-(4-((1,4-diazepan-1-yl)methyl)piperidin-1-yl)-1-methyl-1H-indazol-3-yl)piperidine-2,6-dione (0.25 g) as a brown solid. LC-MS (ESI): m/z=451.29 [M+H]⁺.

### Preparation of 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-1,4-diazepan-1-yl)methyl)piperidin-1-yl)-1-methyl-1H-indazol-3-yl)piperidine-2,6-dione

To a stirred solution of 3-(7-(4-((1,4-diazepan-1-yl)methyl)piperidin-1-yl)-1-methyl-1H-indazol-3-yl)piperidine-2,6-dione (0.25 g, 0.55 mmol) in DMSO (1.2 mL), DIPEA (0.96 mL) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.05 g, 0.10 mmol) were added. The reaction was stirred at 100° C. for 16 hours. The solvent was removed under vacuum to obtain a crude product.

The crude product was purified by prep HPLC to afford 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-1,4-diazepan-1-yl)methyl)piperidin-1-yl)-1-methyl-1H-indazol-3-yl)piperidine-2,6-dione (0.036 g) as an off-white solid. 1H NMR (400 MHz, DMSO-d6): δ 10.87 (s, 1H), 8.72 (s, 1H), 8.19 (s, 1H), 8.03 (s, 1H), 7.79 (d, J=7.60 Hz, 1H), 7.43 (d, J=9.20 Hz, 1H), 7.36 (d, J=8.80 Hz, 1H), 7.02-6.99 (m, 2H), 6.16 (s, 1H), 4.45-4.31 (m, 3H), 4.21 (s, 3H), 3.80-3.60 (m, 7H), 3.18 (br, 3H), 2.66-2.60 (m, 8H), 2.31-2.33 (m, 3H), 2.18-2.13 (m, 1H), 1.75 (br s, 4H), 1.60 (s, 1H), 1.36-1.24 (m, 3H), 0.71 (d, J=7.60 Hz, 1H), 0.51 (t, J=5.60 Hz, 2H), 0.36-0.33 (m, 1H). LC-MS (ESI): m/z=870.53 [M+H]⁺.

### Example 26: 3-(6-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione (Compound 414a)

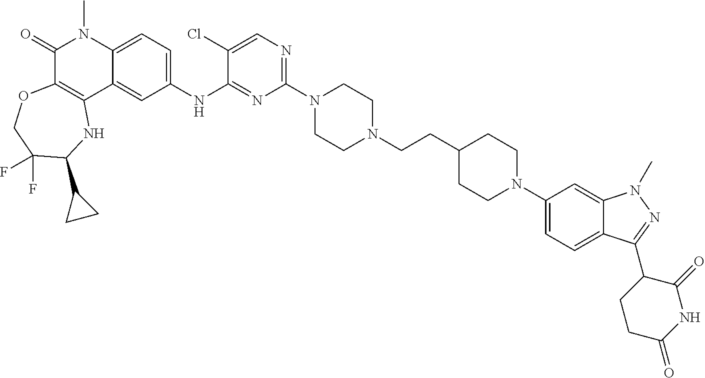 [SMILES: Cn1nc(C2CCC(=O)NC2=O)c2ccc(N3CCC(CCN4CCN(c5ncc(Cl)c(Nc6ccc7c(c6)c6c(c(=O)n7C)OCC(F)(F)[C@H](C7CC7)N6)n5)CC4)CC3)cc21]

### Preparation of 2-(1-(3-(2,6-bis(benzyloxy)pyridin-3-yl)-1-methyl-1H-indazol-6-yl)piperidin-4-yl)ethan-1-ol

To a stirred suspension of 3-(2,6-bis(benzyloxy)pyridin-3-yl)-6-bromo-1-methyl-1H-indazole (2.5 g, 4.99 mmol) in 1,4-dioxane (50 mL) was added 2-(piperidin-4-yl)ethan-1-ol (0.97 g, 7.48 mmol) and cesium carbonate (4.87 g, 14.97 mmol). After purging with argon gas for 15 minutes, RuPhos (0.23 g, 0.5 mmol) and RuPhos-Pd-G3 (0.21 g, 0.25 mmol) were added. The reaction was heated at 100° C. for 5 hours. The reaction mixture was filtered through celite and concentrated under reduced pressure. The crude product was purified by column chromatography (65% ethyl acetate in petroleum ether) to afford 2-(1-(3-(2,6-bis(benzyloxy)pyridin-3-yl)-1-methyl-1H-indazol-6-yl)piperidin-4-yl)ethan-1-ol (0.75 g) as a yellow semi-solid. LC-MS (ESI): m/z=549.63 [M+H]⁺.

### Preparation of 2-(1-(3-(2,6-bis(benzyloxy)pyridin-3-yl)-1-methyl-1H-indazol-6-yl)piperidin-4-yl)ethyl methanesulfonate

A stirred solution of 2-(1-(3-(2,6-bis(benzyloxy)pyridin-3-yl)-1-methyl-1H-indazol-6-yl)piperidin-4-yl)ethan-1-ol (0.75 g, 1.37 mmol)) in DCM was cooled to 0° C. TEA (0.57 mL, 4.1 mmol) and methanesulfonyl chloride (0.32 mL, 4.1 mmol) was added and the reaction was stirred for 2 hours. The reaction mixture was concentrated under reduced pressure to afford a crude product. The crude product was washed with n-pentane to afford 2-(1-(3-(2,6-bis(benzyloxy)pyridin-3-yl)-1-methyl-1H-indazol-6-yl)piperidin-4-yl)ethyl methanesulfonate (1.0 g) as an orange semi-solid. LC-MS (ESI): m/z=627.37 [M+H]⁺.

### Preparation of tert-butyl 4-(2-(1-(3-(2,6-bis(benzyloxy)pyridin-3-yl)-1-methyl-1H-indazol-6-yl)piperidin-4-yl)ethyl)piperazine-1-carboxylate

To a stirred solution of 2-(1-(3-(2,6-bis(benzyloxy)pyridin-3-yl)-1-methyl-1H-indazol-6-yl)piperidin-4-yl)ethyl methanesulfonate (0.7 g, 1.12 mmol) and tert-butyl piperazine-1-carboxylate (0.42 g, 2.24 mmol) in DMSO (5 mL) was added N,N-diisopropylethylamine (1.59 mL, 8.96 mmol). The reaction mixture was stirred at 50° C. for 16 hours. The reaction mixture was diluted with ice water (10 mL) and extracted with ethyl acetate (2×20 mL). The separated organic layers were washed with brine solution, dried over anhydrous sodium sulfate, filtered, and concentrated to afford a crude product. The crude product was purified by column chromatography (74% ethyl acetate in petroleum ether) to afford tert-butyl 4-(2-(1-(3-(2,6-bis(benzyloxy)pyridin-3-yl)-1-methyl-1H-indazol-6-yl)piperidin-4-yl)ethyl)piperazine-1-carboxylate (0.7 g) as an off-white solid. LC-MS (ESI): m/z=717.75 [M+H]⁺.

### Preparation of tert-butyl 4-(2-(1-(3-(2,6-dioxopiperidin-3-yl)-1-methyl-1H-indazol-6-yl)piperidin-4-yl)ethyl)piperazine-1-carboxylate

To a stirred solution of tert-butyl 4-(2-(1-(3-(2,6-bis(benzyloxy)pyridin-3-yl)-1-methyl-1H-indazol-6-yl)piperidin-4-yl)ethyl)piperazine-1-carboxylate (0.70 g, 0.97 mmol) in THF (21 mL) was added 20% Pd(OH)₂ on carbon (0.90 g) and acetic acid (0.4 mL). The reaction was put under H₂ atmosphere (80 psi) and stirred at room temperature for 8 hours. The reaction mixture was filtered through celite and concentrated to obtain a crude product. The crude was washed with n-pentane and dried to obtain tert-butyl 4-(2-(1-(3-(2,6-dioxopiperidin-3-yl)-1-methyl-1H-indazol-6-yl)piperidin-4-yl)ethyl)piperazine-1-carboxylate (0.55 g) as a green semi-solid. LC-MS (ESI): m/z=539.63 [M+H]⁺.

### Preparation of 3-(1-methyl-6-(4-(2-(piperazin-1-yl)ethyl)piperidin-1-yl)-1H-indazol-3-yl)piperidine-2,6-dione

A stirred solution of tert-butyl 4-(2-(1-(3-(2,6-dioxopiperidin-3-yl)-1-methyl-1H-indazol-6-yl)piperidin-4-yl)ethyl)piperazine-1-carboxylate (0.55 g, 1.02 mmol)) in DCM (6 mL) was cooled to 0° C. TFA (5.5 mL) was added, and the reaction was allowed to warm to room temperature with stirring over 2 hours. The reaction mixture was concentrated under reduced pressure to afford a crude product. The crude product was washed with n-pentane to obtain 3-(1-methyl-6-(4-(2-(piperazin-1-yl)ethyl)piperidin-1-yl)-1H-indazol-3-yl)piperidine-2,6-dione (0.3 g) as a brown solid. LC-MS (ESI): m/z=439.55 [M+H]⁺.

### Preparation of 3-(6-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

To a stirred solution of 3-(1-methyl-6-(4-(2-(piperazin-1-yl)ethyl)piperidin-1-yl)-1H-indazol-3-yl)piperidine-2,6-dione (0.15 g, 0.34 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.1 g, 0.21 mmol) in DMSO (3 mL), N,N-diisopropylethylamine (0.35 g, 2.72 mmol) was added.

The reaction mixture was stirred at 100° C. for 5 hours. The solvent was removed under vacuum to obtain a crude product. The crude product was purified by prep HPLC to afford 3-(6-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione (48 mg) as an off-white solid. 1H NMR (400 MHz, DMSO-d6): δ 10.90 (s, 1H), 8.84 (s, 1H), 8.22-8.20 (m, 1H), 8.04 (s, 1H), 7.69 (dd, J=1.60, 9.00 Hz, 1H), 7.45 (dd, J=9.20, 12.60 Hz, 2H), 6.89 (dd, J=8.80, Hz, 1H), 6.81 (s, 1H), 6.22 (s, 1H), 4.50-4.40 (m, 2H), 4.24-4.22 (m, 1H), 3.88 (s, 3H), 3.56 (s, 7H), 3.21 (s, 1H), 2.69-2.67 (m, 2H), 2.62-2.60 (m, 2H), 2.50-2.35 (m, 6H), 2.30-2.28 (m, 2H), 1.91 (s, 1H), 1.76 (d, J=12.00 Hz, 3H), 1.41-1.36 (m, 6H), 0.70 (br s, 1H), 0.52-0.50 (m, 2H), 0.36-0.35 (m, 1H). LC-MS (ESI): m/z=870.52 [M+H]⁺.

### Example 27: 3-(7-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione (Compound 415a)

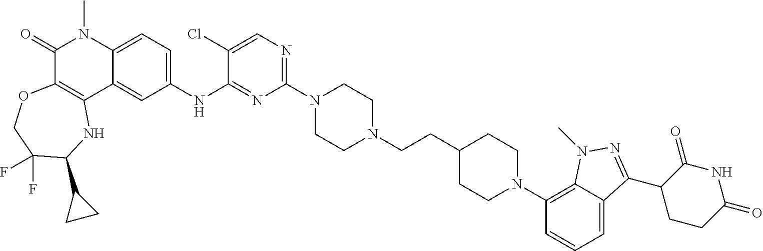 [SMILES: Cn1nc(C2CCC(=O)NC2=O)c2cccc(N3CCC(CCN4CCN(c5ncc(Cl)c(Nc6ccc7c(c6)c6c(c(=O)n7C)OCC(F)(F)[C@H](C7CC7)N6)n5)CC4)CC3)c21]

### Preparation of 2-(1-(3-(2,6-bis(benzyloxy)pyridin-3-yl)-1-methyl-1H-indazol-7-yl)piperidin-4-yl)ethan-1-ol

To a stirred suspension of 3-(2,6-bis(benzyloxy)pyridin-3-yl)-7-bromo-1-methyl-1H-indazole (2.0 g, 4.0 mmol) in 1,4-dioxane (50 mL) was added 2-(piperidin-4-yl)ethan-1-ol (0.77 g, 6.0 mmol) and cesium carbonate (3.91 g, 12.0 mmol). After purging with argon gas for 15 minutes, Pd-PEPPSI-iHeptCl (0.19 g, 0.20 mmol) was added, and the reaction was heated at 100° C. for 5 hours.

The reaction mixture was filtered through celite and concentrated under reduced pressure to afford a crude product. The crude was purified by column chromatography (65% ethyl acetate in petroleum ether) to afford 2-(1-(3-(2,6-bis(benzyloxy)pyridin-3-yl)-1-methyl-1H-indazol-7-yl)piperidin-4-yl)ethan-1-ol (0.70 g) as a yellow solid. LC-MS (ESI): m/z=549.59 [M+H]⁺.

### Preparation of 2-(1-(3-(2,6-bis(benzyloxy)pyridin-3-yl)-1-methyl-1H-indazol-7-yl)piperidin-4-yl)ethyl methanesulfonate

A stirred solution of 2-(1-(3-(2,6-bis(benzyloxy)pyridin-3-yl)-1-methyl-1H-indazol-7-yl)piperidin-4-yl)ethan-1-ol (0.70 g, 1.27 mmol)) in DCM cooled to 0° C. TEA (0.53 mL, 3.81 mmol) and methanesulfonyl chloride (0.30 mL, 3.81 mmol) were added, and the reaction was allowed to warm to room temperature over 2 hours. The reaction mixture was concentrated under reduced pressure to afford a crude product. The crude product was washed with n-pentane to afford 2-(1-(3-(2,6-bis(benzyloxy)pyridin-3-yl)-1-methyl-1H-indazol-7-yl)piperidin-4-yl)ethyl methanesulfonate (1.0 g) as an orange semi-solid. LC-MS (ESI): m/z=627.57 [M+H]⁺.

### Preparation of tert-butyl 4-(2-(1-(3-(2,6-bis(benzyloxy)pyridin-3-yl)-1-methyl-1H-indazol-7-yl)piperidin-4-yl)ethyl)piperazine-1-carboxylate

To a stirred solution of 2-(1-(3-(2,6-bis(benzyloxy)pyridin-3-yl)-1-methyl-1H-indazol-7-yl)piperidin-4-yl)ethyl methanesulfonate (0.7 g, 1.12 mmol) and tert-butyl piperazine-1-carboxylate (0.42 g, 2.24 mmol) in DMSO was added N,N-diisopropylethylamine (1.59 mL, 8.96 mmol). The reaction mixture was then stirred at 50° C. for 16 hours. The reaction mixture was diluted with ice water (70 mL) and extracted with ethyl acetate (2×70 mL). The separated organic layers were washed with brine solution, dried over anhydrous sodium sulfate, filtered, and concentrated to afford a crude product. The crude product was purified by column chromatography (74% ethyl acetate in petroleum ether) to afford tert-butyl 4-(2-(1-(3-(2,6-bis(benzyloxy)pyridin-3-yl)-1-methyl-1H-indazol-7-yl)piperidin-4-yl)ethyl)piperazine-1-carboxylate (0.7 g) as an off-white solid.

### Preparation of tert-butyl 4-(2-(1-(3-(2,6-dioxopiperidin-3-yl)-1-methyl-1H-indazol-7-yl)piperidin-4-yl)ethyl)piperazine-1-carboxylate

A stirred solution of tert-butyl 4-(2-(1-(3-(2,6-bis(benzyloxy)pyridin-3-yl)-1-methyl-1H-indazol-7-yl)piperidin-4-yl)ethyl)piperazine-1-carboxylate (0.7 g, 0.97 mmol) in THF (21 mL) was degassed with nitrogen and then 20% Pd(OH)₂ on carbon (0.90 g) was added. The reaction mixture was put under H₂ atmosphere (80 psi) and stirred at room temperature for 16 hours. The reaction mixture was filtered through celite and concentrated to obtain crude product. The crude product was washed with n-pentane and dried to afford tert-butyl 4-(2-(1-(3-(2,6-dioxopiperidin-3-yl)-1-methyl-1H-indazol-7-yl)piperidin-4-yl)ethyl)piperazine-1-carboxylate (0.55 g) as a green solid.

LC-MS (ESI): m/z=539.63 [M+H]⁺.

### Preparation of 3-(1-methyl-7-(4-(2-(piperazin-1-yl)ethyl)piperidin-1-yl)-1H-indazol-3-yl)piperidine-2,6-dione

A stirred solution of tert-butyl 4-(2-(1-(3-(2,6-dioxopiperidin-3-yl)-1-methyl-1H-indazol-7-yl)piperidin-4-yl)ethyl)piperazine-1-carboxylate (0.50 g,0.92 mmol) in DCM (5.0 mL) was cooled to 0° C. and TFA (5.0 mL) was added. The reaction mixture was allowed to warm to room temperature with stirring over 2 hours. The reaction mixture was concentrated under reduced pressure to remove solvent. The crude product was washed with n-pentane to obtain 3-(1-methyl-7-(4-(2-(piperazin-1-yl)ethyl)piperidin-1-yl)-1H-indazol-3-yl)piperidine-2,6-dione (0.3 g) as a brown solid. LC-MS (ESI): m/z=439.55 [M+H]⁺.

### Preparation of 3-(7-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione

To a stirred solution of 3-(1-methyl-7-(4-(2-(piperazin-1-yl)ethyl)piperidin-1-yl)-1H-indazol-3-yl)piperidine-2,6-dione (0.15 g, 0.34 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.1 g, 0.21 mmol) in DMSO (3 mL), N,N-diisopropylethylamine (0.35 g, 2.72 mmol) was added.

The reaction mixture was stirred at 100° C. for 5 hours. The reaction mixture was poured into ice cold water and stirred for 15 minutes. The resulting precipitate was filtered and dried to obtain a crude product. The crude product was purified by prep HPLC to obtain 3-(7-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)ethyl)piperidin-1-yl)-1-methyl-1H-indazol-3-yl)piperidine-2,6-dione (0.028 g) as an off-white solid. 1H NMR (400 MHz, DMSO-d6): δ 10.90 (s, 1H), 8.85 (s, 1H), 8.31 (s, 1H), 8.22 (d, J=2.00 Hz, 1H), 8.05 (s, 1H), 7.72 (dd, J=2.00, 9.20 Hz, 1H), 7.46 (d, J=9.20 Hz, 1H), 7.37-7.35 (m, 1H), 6.99-7.01 (m, 2H), 6.22 (s, 1H), 4.44-4.30 (m, 3H), 4.23 (s, 3H), 3.56 (br, 7H), 3.23 (br, 5H), 2.67-2.59 (m, 4H), 2.36-2.30 (m, 7H), 2.18-2.14 (m, 2H), 1.80-1.82 (m, 2H), 1.46-1.35 (m, 5H), 0.73-0.72 (m, 1H), 0.53-0.52 (m, 2H), 0.36-0.35 (m, 1H). LC-MS (ESI): m/z=870.52 [M+H]⁺.

### Example 28: 3-(7-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (Compound

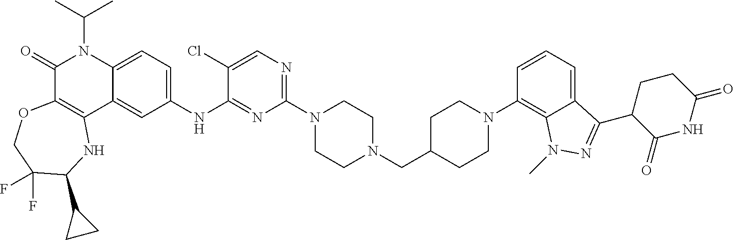 [SMILES: CC(C)n1c(=O)c2c(c3cc(Nc4nc(N5CCN(CC6CCN(c7cccc8c(C9CCC(=O)NC9=O)nn(C)c78)CC6)CC5)ncc4Cl)ccc31)N[C@@H](C1CC1)C(F)(F)CO2]

### Preparation of 2-(isopropylamino)-5-nitrobenzoic acid

A stirred solution of 2-fluoro-5-nitrobenzoic acid (50 g, 270.11 mmol) in pyridine (500 mL) was put under N2 atmosphere. Propan-2-amine (0.42 g, 4.38 mmol) was added dropwise, and then the reaction mixture was stirred at 60° C. for 16 hours. The reaction mixture was quenched with 1N HCl and extracted with ethyl acetate. The collected organic layers were washed with brine solution, dried over anhydrous sodium sulfate, filtered, and concentrated to obtain crude compound. The crude product was purified by flash column chromatography (20% ethyl acetate in petroleum ether) to afford 2-(isopropylamino)-5-nitrobenzoic acid (65.0 g) as a yellow solid. LC-MS (ESI): m/z=225.17 [M+H]⁺.

### Preparation of 1-isopropyl-6-nitro-2H-benzo[d][1,3]oxazine-2,4(1H)-dione

To a stirred solution of 2-(isopropylamino)-5-nitrobenzoic acid (23 g, 102.67 mmol) in THF (230 mL), bis(trichloromethyl)carbonate (32.02 g, 107.81 mmol) was added dropwise. The reaction mixture was then heated at 60° C. for 24 hours. Solvent was removed under reduced pressure to obtain 1-isopropyl-6-nitro-2H-benzo[d][1,3]oxazine-2,4(1H)-dione (20.0 g) as a yellow solid. LC-MS (ESI): m/z=250.22 [M+H]⁺.

### Preparation of ethyl 4-hydroxy-1-isopropyl-6-nitro-2-oxo-1,2-dihydroquinoline-3-carboxylate

A stirred solution of 1-isopropyl-6-nitro-2H-benzo[d][1,3]oxazine-2,4(1H)-dione (15.0 g, 59.76 mmol) and diethyl malonate (28.7 g, 179.28 mmol) in THF (150 mL) was cooled to 0° C. under N2 atmosphere. NaH (4.30 g, 179.28 mmol) was added and the resulting reaction mixture was allowed to warm to room temperature over 48 hours with stirring. The reaction was quenched with 1N HCl and extracted with ethyl acetate. The separated organic layers were washed with brine solution and evaporated to obtain a crude product. The crude product was purified by triturating with diethyl ether and then n-hexane to afford ethyl 4-hydroxy-1-isopropyl-6-nitro-2-oxo-1,2-dihydroquinoline-3-carboxylate (12.0 g) as a yellow solid. LC-MS (ESI): m/z=321.41 [M+H]⁺.

### Preparation of ethyl 4-chloro-1-isopropyl-6-nitro-2-oxo-1,2-dihydroquinoline-3-carboxylate

A stirred solution of ethyl 4-hydroxy-1-isopropyl-6-nitro-2-oxo-1,2-dihydroquinoline-3-carboxylate (25 g, 78.05 mmol) and phosphorus oxychloride (125.0 mL) was refluxed at 80° C. for 16 hours. The reaction mixture was concentrated under reduced pressure. The resulting residue was quenched with a saturated solution of sodium bicarbonate and extracted with ethyl acetate. The separated organic layers were washed with brine solution, dried over anhydrous sodium sulfate, filtered, and concentrated to obtain crude compound. The crude product was purified by triturating with diethyl ether and then n-hexane to afford ethyl 4-chloro-1-isopropyl-6-nitro-2-oxo-1,2-dihydroquinoline-3-carboxylate (15.0 g) as a brown solid. LC-MS (ESI): m/z=339.29 [M+H]⁺.

### Preparation of ethyl (S)-4-((1-cyclopropyl-2,2-difluoro-3-hydroxypropyl)amino)-1-isopropyl-6-nitro-2-oxo-1,2-dihydroquinoline-3-carboxylate

DIPEA (4.9 mL, 26.62 mmol) was added to a stirred solution of (S)-3-amino-3-cyclopropyl-2,2-difluoropropan-1-ol hydrochloride (synthesized using published methods, e.g., International Application Publication Nos. WO 2019/197842 and WO 2021/074620) (3.2 g, 17.75 mmol) and ethyl 4-chloro-1-isopropyl-6-nitro-2-oxo-1,2-dihydroquinoline-3-carboxylate (3.0 g, 8.87 mmol) in toluene (30.0 mL). The reaction mixture was heated at 110° C. for 24 hours. The reaction mixture was quenched with water and extracted into ethyl acetate. The combined organic layers were washed with brine solution, dried over anhydrous sodium sulfate, filtered, and concentrated under vacuum to obtain a crude product. The crude product was purified by flash column chromatography (30% ethyl acetate in petroleum ether) to afford ethyl (S)-4-((1-cyclopropyl-2,2-difluoro-3-hydroxypropyl)amino)-1-isopropyl-6-nitro-2-oxo-1,2-dihydroquinoline-3-carboxylate (2.20 g) as a yellow solid. LC-MS (ESI): m/z=454.35 [M+H]⁺.

### Preparation of (S)-4-((1-cyclopropyl-2,2-difluoro-3-hydroxypropyl)amino)-1-isopropyl-6-nitroquinolin-2(1H)-one

To a stirred solution of ethyl (S)-4-((1-cyclopropyl-2,2-difluoro-3-hydroxypropyl)amino)-1-isopropyl-6-nitro-2-oxo-1,2-dihydroquinoline-3-carboxylate (2.5 g, 5.51 mmol) in MeCN (12.5 mL), a 2.0 M NaOH solution (12.5 mL) was added. The reaction mixture was stirred at 90° C. for 16 hours. The reaction mixture was poured into water and acidified with 6N HCl. The resulting mixture was extracted into ethyl acetate. The combined organic layers were washed with brine solution, dried over anhydrous sodium sulfate, filtered, and concentrated under vacuum to obtain a crude product. The crude product was purified by flash column chromatography (30% ethyl acetate in petroleum ether) to afford (S)-4-((1-cyclopropyl-2,2-difluoro-3-hydroxypropyl)amino)-1-isopropyl-6-nitroquinolin-2(1H)-one (1.95 g) as a pale yellow solid. LC-MS (ESI): m/z=382.39 [M+H]⁺.

### Preparation of (S)-3-bromo-4-((1-cyclopropyl-2,2-difluoro-3-hydroxypropyl)amino)-1-isopropyl-6-nitroquinolin-2(1H)-one

TFA (2.75 mL) was added to a stirred solution of (S)-4-((1-cyclopropyl-2,2-difluoro-3-hydroxypropyl)amino)-1-isopropyl-6-nitroquinolin-2(1H)-one (2.5 g, 7.39 mmol) and NBS (1.17 g, 6.55 mmol) in DCM at 0° C. The reaction mixture was stirred at 0° C. for 15 minutes. The reaction mixture quenched with water and extracted into DCM. The combined organic layers were washed with NaHCO₃ solution, washed with brine solution, dried over anhydrous sodium sulfate, filtered, and concentrated under vacuum to obtain a crude product. The crude product was purified by flash column chromatography (20% ethyl acetate in petroleum ether) to afford (S)-3-bromo-4-((1-cyclopropyl-2,2-difluoro-3-hydroxypropyl)amino)-1-isopropyl-6-nitroquinolin-2(1H)-one (2.5 g) as an off-white/yellow solid. LC-MS (ESI): m/z=458.29 [M−H]⁻.

### Preparation of (S)-2-cyclopropyl-3,3-difluoro-7-isopropyl-10-nitro-1,2,3,4-tetrahydro-[1,4]oxazepino[2,3-c]quinolin-6(7H)-one

Lithium tert-butoxide in THF (19.04 mL, 1.0 M, 19.04 mmol) was added to a stirred solution of (S)-3-bromo-4-((1-cyclopropyl-2,2-difluoro-3-hydroxypropyl)amino)-1-isopropyl-6-nitroquinolin-2(1H)-one (2.5 g, 5.44 mmol) in THF at room temperature. The reaction mixture was then refluxed at 60° C. for 5 hours. The reaction mixture quenched with water and extracted into ethyl acetate. The combined organic layers were washed with brine solution, dried over anhydrous sodium sulfate, filtered, and concentrated under vacuum to obtain a crude product. The crude product was purified by flash column chromatography (50% ethyl acetate in petroleum ether) to afford (S)-2-cyclopropyl-3,3-difluoro-7-isopropyl-10-nitro-1,2,3,4-tetrahydro-[1,4]oxazepino[2,3-c]quinolin-6(7H)-one (1.70 g) as a pale yellow solid. LC-MS (ESI): m/z=380.37 [M+H]⁺.

### Preparation of (S)-10-amino-2-cyclopropyl-3,3-difluoro-7-isopropyl-1,2,3,4-tetrahydro-[1,4]oxazepino[2,3-c]quinolin-6(7H)-one

(S)-2-cyclopropyl-3,3-difluoro-7-isopropyl-10-nitro-1,2,3,4-tetrahydro-[1,4]oxazepino[2,3-c]quinolin-6(7H)-one (1.7 g, 4.48 mmol) was dissolved in EtOH (17.0 mL) at room temperature. Palladium on activated carbon, 10% Pd, dry (1.7 g, 100% w/w) was added and the reaction mixture was put under hydrogen pressure (40 psi). The reaction was then heated at 60° C. for 5 hours. The reaction mixture was filtered through elite and washed with ethyl acetate. The filtrate was evaporated to obtain a crude product. Two 1.7 g batches were combined and purified by chiral SFC (Chiralcel OX—H (4.6×250) mm, 5; CO₂: 70%; MeOH: 30%) to afford (S)-10-amino-2-cyclopropyl-3,3-difluoro-7-isopropyl-1,2,3,4-tetrahydro-[1,4]oxazepino[2,3-c]quinolin-6(7H)-one (1.25 g) as a pale yellow solid. LC-MS (ESI): m/z=350.35 [M+H]⁺.

### Preparation of (S)-2-cyclopropyl-10-((2,5-dichloropyrimidin-4-yl)amino)-3,3-difluoro-7-isopropyl-1,2,3,4-tetrahydro-[1,4]oxazepino[2,3-c]quinolin-6(7H)-one

To a stirred suspension of (S)-10-amino-2-cyclopropyl-3,3-difluoro-7-isopropyl-1,2,3,4-tetrahydro-[1,4]oxazepino[2,3-c]quinolin-6(7H)-one (0.4 g, 1.14 mmol) in DMSO (0.8 mL), N,N-diisopropylethylamine (0.6 mL, 3.43 mol) and 2,4,5-trichloropyrimidine (0.21 g, 1.14 mmol) were added at room temperature. The reaction was heated at 100° C. for 4 hours. The reaction mixture was allowed to cool to room temperature and was quenched with ice water. The resulting precipitate was filtered to afford (S)-2-cyclopropyl-10-((2,5-dichloropyrimidin-4-yl)amino)-3,3-difluoro-7-isopropyl-1,2,3,4-tetrahydro-[1,4]oxazepino[2,3-c]quinolin-6(7H)-one (0.48 g) as a light yellow solid. LC-MS (ESI): m/z=495.37 [M+H]⁺.

### Preparation of 3-(7-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione

A stirred solution of 2-(2,6-dioxopiperidin-3-yl)-4-(4-(piperazin-1-ylmethyl)piperidin-1-yl)isoindoline-1,3-dione hydrochloride (0.45 g, 0.94 mmol) in DMSO (0.6 mL) was cooled to 0° C. and put under N2 atmosphere. DIPEA (0.52 mL, 2.83 mmol) was added and the reaction mixture was stirred for 10 minutes. (S)-2-cyclopropyl-10-((2,5-dichloropyrimidin-4-yl)amino)-3,3-difluoro-7-isopropyl-1,2,3,4-tetrahydro-[1,4]oxazepino[2,3-c]quinolin-6(7H)-one (0.23 g, 0.47 mmol) was added and the resulting reaction mixture was heated to 100° C. for 6 hours. Solvent was removed under vacuum to obtain a crude product. The crude product was purified by prep HPLC to afford 3-(7-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (0.194 mg) as an off-white solid. ¹H NMR (400 MHz, DMSO-d₆): δ 10.9 (s, 1H), 8.80 (s, 1H), 8.24 (s, 2H), 8.11 (s, 1H), 7.68-7.62 (m, 2H), 7.37-7.35 (m, 1H), 7.01 (dd, J=7.6 Hz & 5.6 Hz, 2H), 6.07 (s, 1H), 5.30 (brs, 1H), 4.41-4.31 (m, 3H), 4.23 (s, 3H), 3.67 (m, 4H), 3.16 (m, 3H), 3.24-3.21 (m, 3H), 2.69-2.62 (m, 4H), 2.35-2.18 (m, 4H), 1.92-1.85 (m, 2H), 1.61 (s, 1H), 1.52 (d, J=8.0 Hz 6H), 1.35-1.32 (m, 3H), 0.71-0.64 (m, 1H), 0.53 (m, 2H), 0.40-0.38 (m, 1H). LC-MS (ESI): m/z=884.58 [M+H]⁺.

### Example 29: 3-(6-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (Compound 404a)

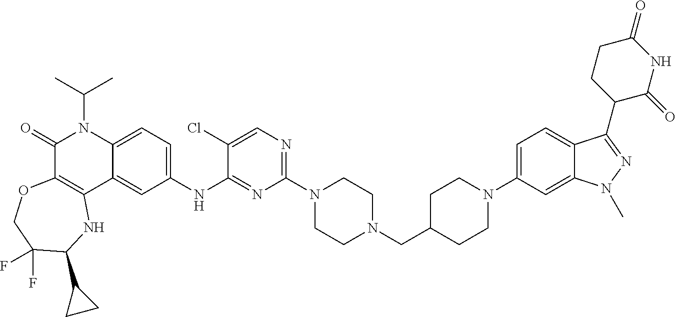 [SMILES: CC(C)n1c(=O)c2c(c3cc(Nc4nc(N5CCN(CC6CCN(c7ccc8c(C9CCC(=O)NC9=O)nn(C)c8c7)CC6)CC5)ncc4Cl)ccc31)N[C@@H](C1CC1)C(F)(F)CO2]

### Preparation of 3-(6-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione

To a stirred solution of 3-(1-methyl-6-(4-(piperazin-1-ylmethyl) piperidin-1-yl)-1H-indazol-3-yl) piperidine-2,6-dione hydrochloride (0.15 g, 0.32 mmol) in DMSO (6.0 mL), (S)-2-cyclopropyl-10-((2,5-dichloropyrimidin-4-yl)amino)-3,3-difluoro-7-isopropyl-1,2,3,4-tetrahydro-[1,4]oxazepino[2,3-c]quinolin-6(7H)-one (0.08 g, 0.16 mmol) and DIPEA (0.30 mL, 1.62 mmol) were added. The reaction mixture was heated at 100° C. for 6 hours. Solvent was removed under vacuum to obtain a crude product. The crude product was purified by prep HPLC to afford 3-(6-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (0.034 g) as an off-white solid. 1H NMR (400 MHz, DMSO-d₆): δ 8.37 (s, 1H), 8.22-8.22 (d, J=2.0 Hz, 1H), 7.71-7.65 (m, 2H), 7.49-7.47 (d, J=8.8 Hz, 1H), 6.92-6.90 (d, J=8.4 Hz, 1H), 6.81 (s, 1H), 6.10 (bs, 1H), 5.32 (bs, 1H), 4.40-4.24 (m, 3H), 3.88 (s, 3H), 3.79-3.76 (d, J=11.6 Hz, 2H), 3.60 (bs, 4H), 3.32 (bs, 1H), 2.63-2.59 (m, 4H), 2.36-2.32 (m, 6H), 2.18-2.16 (d, J=6.8 Hz, 3H), 1.82-1.79 (d, J=12.0 Hz, 3H), 1.52-1.49 (m, 6H), 1.28-1.23 (d, J=10.6 Hz, 3H), 0.70 (bs, 1H), 0.52-0.50 (m, 2H), 0.37-0.36 (m, 1H). LC-MS (ESI): m/z=884.56 [M+H]⁺.

### Example 30: 3-(6-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)propan-2-yl)piperidin-1-yl)-1-methyl-1H-indazol-3-yl)piperidine-2,6-dione (Compound 405a)

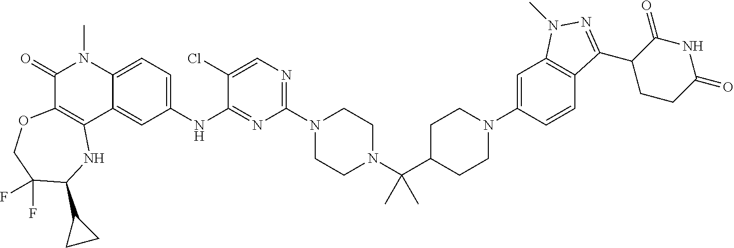 [SMILES: Cn1nc(C2CCC(=O)NC2=O)c2ccc(N3CCC(C(C)(C)N4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-(2-(1-(3-(2,6-bis(benzyloxy)pyridin-3-yl)-1-methyl-1H-indazol-6-yl)piperidin-4-yl)propan-2-yl)piperazine-1-carboxylate

A solution of tert-butyl 4-(2-(piperidin-4-yl)propan-2-yl)piperazine-1-carboxylate (0.30 g, 0.59 mmol), 3-(2,6-bis(benzyloxy)pyridin-3-yl)-6-bromo-1-methyl-1H-indazole (0.37 g, 1.19 mmol) and cesium carbonate (0.58 g, 1.79 mmol) in 1,4-dioxane (20 mL) was degassed with argon for 5 minutes. RuPhos-Pd-G3 (0.02 g, 0.02 mmol) and RuPhos (0.03 g, 0.5 mmol) were added, and the reaction was degassed for an additional 5 minutes. The reaction mixture was heated at 100° C. for 16 hours. The reaction mixture was quenched with ice-cold water (50 mL) and extracted into ethyl acetate (3×100 mL). The combined organic layers were washed with brine solution, dried over anhydrous sodium sulfate, filtered, and concentrated under vacuum to obtain a crude product.

The crude product was purified by column chromatography (40% ethyl acetate in petroleum ether) to afford tert-butyl 4-(2-(1-(3-(2,6-bis(benzyloxy)pyridin-3-yl)-1-methyl-1H-indazol-6-yl)piperidin-4-yl)propan-2-yl)piperazine-1-carboxylate (0.4 g) as a pale brown semi-solid. LC-MS (ESI): m/z=731.86 [M+H]⁺.

### Preparation of tert-butyl 4-(2-(1-(3-(2,6-dioxopiperidin-3-yl)-1-methyl-1H-indazol-6-yl)piperidin-4-yl)propan-2-yl)piperazine-1-carboxylate

To a stirred solution of tert-butyl 4-(2-(1-(3-(2,6-bis(benzyloxy)pyridin-3-yl)-1-methyl-1H-indazol-6-yl)piperidin-4-yl)propan-2-yl)piperazine-1-carboxylate (0.40 g, 0.54 mmol) in THF (8 mL), 20% Pd(OH)₂ on carbon (0.40 g, 100% w/w) was added. The reaction was put under hydrogen atmosphere (20 psi) and stirred at room temperature for 8 hours. The reaction mixture was diluted with DCM (80 mL) and filtered through celite. The filtrate was concentrated under vacuum to obtain tert-butyl 4-(2-(1-(3-(2,6-dioxopiperidin-3-yl)-1-methyl-1H-indazol-6-yl)piperidin-4-yl)propan-2-yl)piperazine-1-carboxylate (0.25 g) as a brown solid. LC-MS (ESI): m/z=553.69 [M+H]⁺.

### Preparation of 3-(1-methyl-6-(4-(2-(piperazin-1-yl)propan-2-yl)piperidin-1-yl)-1H-indazol-3-yl)piperidine-2,6-dione (TFA salt)

To a stirred solution of tert-butyl 4-(2-(1-(3-(2,6-dioxopiperidin-3-yl)-1-methyl-1H-indazol-6-yl)piperidin-4-yl)propan-2-yl)piperazine-1-carboxylate (0.25 g, 0.50 mmol) in DCM (5 mL), TFA (0.7 mL) was added. The reaction was stirred at room temperature for 1 hour. The reaction mixture was concentrated and co-distilled with petroleum ether to obtain 3-(1-methyl-6-(4-(2-(piperazin-1-yl)propan-2-yl)piperidin-1-yl)-1H-indazol-3-yl)piperidine-2,6-dione (0.24 g, TFA salt). LC-MS (ESI): m/z=453.32 [M+H]⁺.

### Preparation of 3-(6-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)propan-2-yl)piperidin-1-yl)-1-methyl-1H-indazol-3-yl)piperidine-2,6-dione

To a stirred solution of 3-(1-methyl-6-(4-(2-(piperazin-1-yl)propan-2-yl)piperidin-1-yl)-1H-indazol-3-yl)piperidine-2,6-dione (TFA salt) (0.12 g, 0.13 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.03 g, 0.08 mmol) in DMSO (2.4 mL), DIPEA (0.23 mL, 1.36 mmol) was added. The reaction was stirred at 100° C. for 3 hours. Solvent was removed under vacuum to obtain a crude product. The crude product was purified by prep HPLC to obtain 3-(6-(4-(2-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)propan-2-yl)piperidin-1-yl)-1-methyl-1H-indazol-3-yl)piperidine-2,6-dione (0.07 g) as an off-white solid. ¹H NMR (400 MHz, DMSO-d₆): 10.80 (s, 1H), 8.76 (s, 1H), 8.22-8.20 (m, 1H), 8.02 (s, 1H), 7.73 (d, J=8.80 Hz, 1H), 7.50-7.43 (m, 2H), 6.91 (d, J=9.20 Hz, 1H), 6.81 (s, 1H), 6.19 (s, 1H), 4.434.42 (m, 2H), 4.38-4.23 (m, 3H), 3.88 (s, 3H), 3.55 (s, 3H), 3.22-3.17 (m, 5H), 2.57-2.50 (m, 8H), 2.32 (d, 1H), 2.28 (d, 1H), 1.74 (d, 3H), 1.34 (s, 1H), 1.18 (d, 2H), 0.89 (s, 6H), 0.72-0.71 (m, 1H), 0.52.50 (m, 2H), 0.36-0.34 (m, 1H). LC-MS (ESI): m/z=882.47 [M−H]⁻.

### Example 31: 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (Compound 442a)

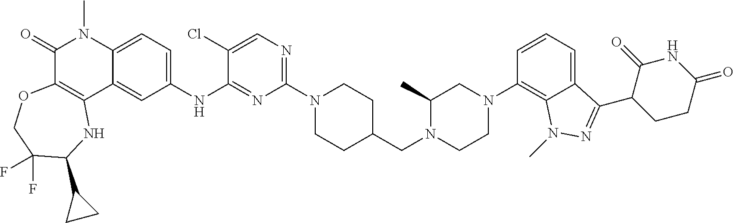 [SMILES: C[C@H]1CN(c2cccc3c(C4CCC(=O)NC4=O)nn(C)c23)CCN1CC1CCN(c2ncc(Cl)c(Nc3ccc4c(c3)c3c(c(=O)n4C)OCC(F)(F)[C@H](C4CC4)N3)n2)CC1]

### Preparation of benzyl (S)-4-((1-(tert-butoxycarbonyl)piperidin-4-yl)methyl)-3-methylpiperazine-1-carboxylate

To a solution of benzyl (S)-3-methylpiperazine-1-carboxylate (2.0 g, 8.54 mmol), tert-butyl 4-formylpiperidine-1-carboxylate (2.18 g, 10.25 mmol), and NaBH(OAc)₃ (3.62 g, 17.08 mmol) in DCM (40 mL), acetic acid (0.51 g, 8.54 mmol) was added. The reaction was stirred at room temperature for 16 hours. The reaction mixture was quenched with ice-cold water (100 mL) and extracted into DCM (3×100 mL). The combined organic layers were washed with brine solution (100 mL), dried over anhydrous sodium sulfate, filtered, and dried under vacuum to obtain a crude product. The crude product was purified by column chromatography (40% ethyl acetate in petroleum ether) to afford benzyl (S)-4-((1-(tert-butoxycarbonyl)piperidin-4-yl)methyl)-3-methylpiperazine-1-carboxylate (3.0 g) as a colorless liquid. LC-MS (ESI): m/z=432.54 [M+H]⁺.

### Preparation of tert-butyl (S)-4-((2-methylpiperazin-1-yl)methyl)piperidine-1-carboxylate

To a stirred solution of benzyl (S)-4-((1-(tert-butoxycarbonyl)piperidin-4-yl)methyl)-3-methylpiperazine-1-carboxylate (3.0 g, 6.95 mmol) in THF (90 mL), 20% Pd(OH)₂ on carbon (3.0 g, 100% w/w) was added. The reaction was put under hydrogen atmosphere (70 psi) and stirred at room temperature for 16 hours. The reaction mixture was filtered through celite, and the filtrate was concentrated to obtain a crude product. The crude product was dissolved in n-pentane and dried to afford tert-butyl (S)-4-((2-methylpiperazin-1-yl)methyl)piperidine-1-carboxylate (2.1 g) as a colorless liquid. LC-MS (ESI): m/z=298.20 [M+H]⁺.

### Preparation of tert-butyl (S)-4-((4-(3-(2,6-bis(benzyloxy)pyridin-3-yl)-1-methyl-1H-indazol-7-yl)-2-methylpiperazin-1-yl)methyl)piperidine-1-carboxylate

A solution of 3-(2,6-bis(benzyloxy)pyridin-3-yl)-7-bromo-1-methyl-1H-indazole (0.70 g, 1.39 mmol), tert-butyl (S)-4-((2-methylpiperazin-1-yl)methyl)piperidine-1-carboxylate (0.62 g, 2.09 mmol) and cesium carbonate (1.36 g, 4.19 mmol) in 1,4-dioxane (14 mL) was degassed with argon for 5 minutes. Pd-PEPPSI-iHeptCl (0.06 g, 0.06 mmol) was added, and the reaction was degassed for an additional 5 minutes. The reaction mixture was heated at 80° C. for 16 hours. The reaction mixture was quenched with ice-cold water (50 mL) and extracted into ethyl acetate (3×100 mL). The combined organic layers were washed with brine solution (50 mL), dried over anhydrous sodium sulfate, filtered, and concentrated under vacuum to obtain a crude product. The crude product was purified by column chromatography (40% ethyl acetate in petroleum ether) to afford tert-butyl (S)-4-((4-(3-(2,6-bis(benzyloxy)pyridin-3-yl)-1-methyl-1H-indazol-7-yl)-2-methylpiperazin-1-yl)methyl)piperidine-1-carboxylate (0.20 g) as a brown semi-solid. LC-MS (ESI): m/z=717.79 [M+H]⁺.

### Preparation of tert-butyl 4-(((2S)-4-(3-(2,6-dioxopiperidin-3-yl)-1-methyl-1H-indazol-7-yl)-2-methylpiperazin-1-yl)methyl)piperidine-1-carboxylate

To a stirred solution of tert-butyl (S)-4-((4-(3-(2,6-bis(benzyloxy)pyridin-3-yl)-1-methyl-1H-indazol-7-yl)-2-methylpiperazin-1-yl)methyl)piperidine-1-carboxylate (0.2 g, 0.27 mmol) in THF (12 mL), 20% Pd(OH)₂ on carbon (0.2 g, 100% w/w) was added. The reaction was put under hydrogen atmosphere (70 psi) and stirred at room temperature for 16 hours. The reaction mixture was filtered through celite, and the filtrate was concentrated to obtain a crude product. The crude product was washed with n-pentane and dried to afford tert-butyl 4-(((2S)-4-(3-(2,6-dioxopiperidin-3-yl)-1-methyl-1H-indazol-7-yl)-2-methylpiperazin-1-yl)methyl)piperidine-1-carboxylate (0.12 g) as a brown solid. LC-MS (ESI): m/z=539.41 [M+H]⁺.

### Preparation of 3-(1-methyl-7-((S)-3-methyl-4-(piperidin-4-ylmethyl)piperazin-1-yl)-1H-indazol-3-yl)piperidine-2,6-dione (TFA salt)

A stirred solution of tert-butyl 4-(((2S)-4-(3-(2,6-dioxopiperidin-3-yl)-1-methyl-1H-indazol-7-yl)-2-methylpiperazin-1-yl)methyl)piperidine-1-carboxylate (0.12 g, 0.22 mmol) in DCM (2.4 mL) was cooled to 0° C. TFA (1.2 mL) was added, and the reaction was allowed to warm to room temperature over 3 hours with stirring. The reaction mixture was concentrated under vacuum to obtain a crude product. The crude product was washed with n-pentane to obtain 3-(1-methyl-7-((S)-3-methyl-4-(piperidin-4-ylmethyl)piperazin-1-yl)-1H-indazol-3-yl)piperidine-2,6-dione (0.20 g, TFA salt) as a brown solid. LC-MS (ESI): m/z=439.55 [M+H]⁺.

### Preparation of 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione

To a stirred solution 3-(1-methyl-7-((S)-3-methyl-4-(piperidin-4-ylmethyl)piperazin-1-yl)-1H-indazol-3-yl)piperidine-2,6-dione (TFA salt) (0.20 g, 0.37 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.07 g, 0.18 mmol) in DMSO (4 mL), N,N-diisopropylethylamine (0.38 g, 2.98 mmol) was added. The reaction mixture was stirred at 100° C. for 16 hours. Solvent was removed under vacuum to obtain a crude product. The crude product was purified by prep HPLC to afford 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (0.030 g) as an off-white solid. 1H NMR (400 MHz, DMSO-d₆): δ 10.90 (s, 1H), 8.36 (s, 1H), 8.19 (d, J=1.60 Hz, 1H), 8.03 (s, 1H), 7.73-7.73 (m, 1H), 7.45 (d, J=9.20 Hz, 1H), 7.38-7.38 (m, 1H), 7.01-7.02 (m, 2H), 6.19 (s, 1H), 4.32-4.33 (m, 5H), 4.24 (s, 3H), 3.57 (s, 3H), 3.22-3.23 (m, 1H), 3.01-3.10 (m, 4H), 2.77-2.80 (m, 4H), 2.58-2.63 (m, 4H), 2.31-2.32 (m, 2H), 2.17-2.19 (m, 1H), 1.60-2.0 (m, 4H), 1.31 (s, 1H), 1.02 (m, 5H), 0.70 (m, 1H), 0.52 (m, 2H), 0.35 (m, 1H). LC-MS (ESI): m/z=868.44 [M−H]⁻.

### Example 32: 3-(6-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (Compound 443b)

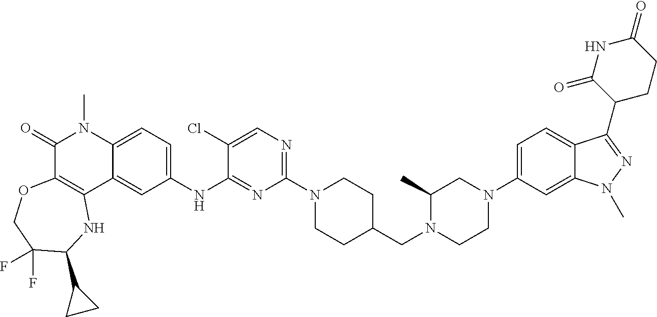 [SMILES: C[C@H]1CN(c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1]

### Preparation of tert-butyl (S)-4-((4-(3-(2,6-bis(benzyloxy)pyridin-3-yl)-1-methyl-1H-indazol-6-yl)-2-methylpiperazin-1-yl)methyl)piperidine-1-carboxylate

A solution of 3-(2,6-bis(benzyloxy)pyridin-3-yl)-6-bromo-1-methyl-1H-indazole (0.50 g, 0.99 mmol), tert-butyl (S)-4-((2-methylpiperazin-1-yl)methyl)piperidine-1-carboxylate (0.44 g, 1.49 mmol), and cesium carbonate (0.97 g, 1.49 mmol) in 1,4-dioxane (10 mL) was degassed with argon for 5 minutes. Pd-PEPPSI-iHeptCl (0.97 g, 0.1 mmol) was added, and the reaction was degassed for an additional 5 minutes. The reaction mixture was heated at 100° C. for 16 hours. The reaction mixture was quenched with ice-cold water (50 mL) and extracted into ethyl acetate (3×100 mL). The combined organic layers were washed with brine solution (50 mL), dried over anhydrous sodium sulfate, filtered, and dried under vacuum to obtain a crude product. The crude product was purified by column chromatography (40% ethyl acetate in petroleum ether) to afford tert-butyl (S)-4-((4-(3-(2,6-bis(benzyloxy)pyridin-3-yl)-1-methyl-1H-indazol-6-yl)-2-methylpiperazin-1-yl)methyl)piperidine-1-carboxylate (0.25 g) as a brown semi-solid. LC-MS (ESI): m/z=717.71 [M+H]⁺.

### Preparation of tert-butyl 4-(((2S)-4-(3-(2,6-dioxopiperidin-3-yl)-1-methyl-1H-indazol-6-yl)-2-methylpiperazin-1-yl)methyl)piperidine-1-carboxylate

To a stirred solution of tert-butyl (S)-4-((4-(3-(2,6-bis(benzyloxy)pyridin-3-yl)-methyl-1H-indazol-6-yl)-2-methylpiperazin-1-yl)methyl)piperidine-1-carboxylate (0.25 g, 0.34 mmol) in THF (15 mL), 20% Pd(OH)₂ on carbon (0.25 g, 100% w/w) was added. The reaction was put under hydrogen atmosphere (70 psi) and stirred at room temperature for 12 hours. The reaction mixture was filtered through celite, and the filtrate was concentrated to obtain a crude product. The crude product was washed with n-pentane and dried to afford tert-butyl 4-(((2S)-4-(3-(2,6-dioxopiperidin-3-yl)-1-methyl-1H-indazol-6-yl)-2-methylpiperazin-1-yl)methyl)piperidine-1-carboxylate (0.15 g) as a brown solid. LC-MS (ESI): m/z=539.71 [M+H]⁺.

### Preparation of 3-(1-methyl-6-((S)-3-methyl-4-(piperidin-4-ylmethyl)piperazin-1-yl)-1H-indazol-3-yl)piperidine-2,6-dione (TFA salt)

A stirred solution of tert-butyl 4-(((2S)-4-(3-(2,6-dioxopiperidin-3-yl)-1-methyl-1H-indazol-6-yl)-2-methylpiperazin-1-yl)methyl)piperidine-1-carboxylate (0.15 g, 0.27 mmol) in DCM (3 mL) was cooled to 0° C. TFA(1.5 mL) was added, and the reaction was allowed to warm to room temperature over 3 hours with stirring. The reaction mixture was concentrated under vacuum to obtain a crude product. The crude product was washed with n-pentane to afford 3-(1-methyl-6-((S)-3-methyl-4-(piperidin-4-ylmethyl)piperazin-1-yl)-1H-indazol-3-yl)piperidine-2,6-dione (0.2 g, TFA salt) as a brown solid. LC-MS (ESI): m/z=439.28 [M+H]⁺.

### Preparation of 3-(6-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione

To a stirred solution 3-(1-methyl-6-((S)-3-methyl-4-(piperidin-4-ylmethyl)piperazin-1-yl)-1H-indazol-3-yl)piperidine-2,6-dione (TFA salt) (0.20 g, 0.37 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.14 mmol) in DMSO (4 mL), N,N-diisopropylethylamine (0.38 g, 2.98 mmol) was added. The reaction mixture was stirred at 100° C. for 16 hours. Solvent was removed under vacuum to obtain a crude product. The crude product was purified by prep HPLC to afford 3-(6-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (0.011 g) as an off-white solid. ¹H NMR (400 MHz, DMSO-d₆): δ 10.90 (s, 1H), 8.85 (s, 1H), 8.14 (s, 1H), 8.06 (s, 1H), 7.74 (d, J=8.00 Hz, 1H), 7.57 (d, J=12.00 Hz, 1H), 7.42 (d, J=8.00 Hz, 1H), 6.95-6.99 (m, 2H), 6.21 (s, 1H), 4.26-4.29 (m, 5H), 3.92 (s, 3H), 3.68-3.71 (m, 1H), 3.56 (s, 3H), 3.25 (m, 4H), 2.89-2.98 (m, 4H), 2.61-2.67 (m, 2H), 2.32 (s, 1H), 2.16-2.18 (m, 3H), 1.90 (m, 2H), 1.35-1.37 (m, 4H), 1.19-1.25 (m, 4H), 0.71-0.73 (m, 1H), 0.51-0.54 (m, 2H), 0.33-0.34 (m, 1H).

LC-MS (ESI): m/z=870.56 [M+H]⁺.

### Example 33: 3-(7-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (Compound 442b)

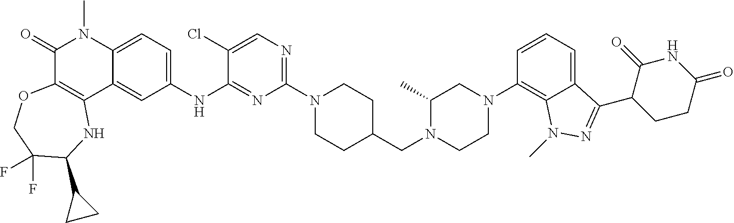 [SMILES: C[C@@H]1CN(c2cccc3c(C4CCC(=O)NC4=O)nn(C)c23)CCN1CC1CCN(c2ncc(Cl)c(Nc3ccc4c(c3)c3c(c(=O)n4C)OCC(F)(F)[C@H](C4CC4)N3)n2)CC1]

### Preparation of benzyl (R)-4-((1-(tert-butoxycarbonyl)piperidin-4-yl)methyl)-3-methylpiperazine-1-carboxylate

To a solution of benzyl (R)-3-methylpiperazine-1-carboxylate (10.0 g, 42.70 mmol), tert-butyl 4-formylpiperidine-1-carboxylate (10.92 g, 51.25 mmol), and NaBH(OAc)₃ (9.49 g, 42.70 mmol) in DCM (200 mL), acetic acid (2.56 g, 42.70 mmol) was added. The reaction was stirred at room temperature for 16 hours. The reaction mixture was diluted with ice-cold water (500 mL) and extracted into DCM (3×250 mL). The combined organic layers were washed with brine solution (500 mL), dried over anhydrous sodium sulfate, filtered, and concentrated under vacuum to obtain a crude product. The crude product was purified by column chromatography (40% ethyl acetate in petroleum ether) to afford benzyl (R)-4-((1-(tert-butoxycarbonyl)piperidin-4-yl)methyl)-3-methylpiperazine-1-carboxylate (6.0 g) as a colorless liquid. LC-MS (ESI): m/z=432.58 [M+H]⁺.

### Preparation of tert-butyl (R)-4-((2-methylpiperazin-1-yl)methyl)piperidine-1-carboxylate

To a stirred solution of benzyl (R)-4-((1-(tert-butoxycarbonyl)piperidin-4-yl)methyl)-3-methylpiperazine-1-carboxylate (6.0 g, 13.92 mmol) in THF (180 mL), 20% Pd(OH)₂ on carbon (3.0 g, 31%) was added. The reaction was put under hydrogen atmosphere (60 psi) and stirred at room temperature for 16 hours. The reaction mixture was filtered through celite and concentrated under reduced pressure to obtain a crude product. The crude product was dissolved in n-pentane and concentrated to afford tert-butyl (R)-4-((2-methylpiperazin-1-yl)methyl)piperidine-1-carboxylate (3.0 g) as a colorless liquid. LC-MS (ESI): m/z=298.53 [M+H]⁺.

### Preparation of tert-butyl (R)-4-((4-(3-(2,6-bis(benzyloxy)pyridin-3-yl)-1-methyl-1H-indazol-7-yl)-2-methylpiperazin-1-yl)methyl)piperidine-1-carboxylate

A solution of 3-(2,6-bis(benzyloxy)pyridin-3-yl)-7-bromo-1-methyl-1H-indazole (1.0 g, 2.0 mmol), tert-butyl (R)-4-((2-methylpiperazin-1-yl)methyl)piperidine-1-carboxylate (2.38 g, 8.0 mmol), and cesium carbonate (1.95 g, 6.0 mmol) in 1,4-dioxane (20 mL) was degassed with argon for 5 minutes. Pd-PEPPSI-iHeptCl (0.97 g, 0.1 mmol) was added, and the reaction was degassed for an additional 5 minutes. The reaction was heated at 80° C. for 16 hours. The reaction mixture was quenched with ice-cold water (50 mL) and extracted into ethyl acetate (3×100 mL). The combined organic layers were washed with brine solution (50 mL), dried over anhydrous sodium sulfate, filtered, and concentrated under vacuum to obtain a crude product. The crude product was purified by column chromatography (40% ethyl acetate in petroleum ether) to afford tert-butyl (R)-4-((4-(3-(2,6-bis(benzyloxy)pyridin-3-yl)-1-methyl-1H-indazol-7-yl)-2-methylpiperazin-1-yl)methyl)piperidine-1-carboxylate (1.0 g) as a pale brown semi-solid. LC-MS (ESI): m/z=717.79 [M+H]⁺.

### Preparation of tert-butyl 4-(((2R)-4-(3-(2,6-dioxopiperidin-3-yl)-1-methyl-1H-indazol-7-yl)-2-methylpiperazin-1-yl)methyl)piperidine-1-carboxylate

To a stirred solution of tert-butyl (R)-4-((4-(3-(2,6-bis(benzyloxy)pyridin-3-yl)-1-methyl-1H-indazol-7-yl)-2-methylpiperazin-1-yl)methyl)piperidine-1-carboxylate (1.0 g, 1.42 mmol) in THF (60 mL), 20% Pd(OH)₂ on carbon (1.0 g) was added. The reaction was put under hydrogen atmosphere (60 psi) and stirred at room temperature for 6 hours. The reaction mixture was filtered through celite and concentrated to obtain a crude product. The crude product was washed with n-pentane and dried to afford tert-butyl 4-(((2R)-4-(3-(2,6-dioxopiperidin-3-yl)-1-methyl-1H-indazol-7-yl)-2-methylpiperazin-1-yl)methyl)piperidine-1-carboxylate (0.70 g) as a brown solid.

LC-MS (ESI): m/z=539.69 [M+H]⁺.

### Preparation of 3-(1-methyl-7-((R)-3-methyl-4-(piperidin-4-ylmethyl)piperazin-1-yl)-1H-indazol-3-yl)piperidine-2,6-dione (TFA salt)

A stirred solution of tert-butyl 4-(((2R)-4-(3-(2,6-dioxopiperidin-3-yl)-1-methyl-1H-indazol-7-yl)-2-methylpiperazin-1-yl)methyl)piperidine-1-carboxylate (0.70 g, 1.33 mmol) in DCM (14 mL) was cooled to 0° C. TFA (7.0 mL) was added, and the reaction was allowed to warm to room temperature over 3 hours with stirring. Solvent was removed under vacuum to obtain a crude product. The crude product was washed with n-pentane to afford 3-(1-methyl-7-((R)-3-methyl-4-(piperidin-4-ylmethyl)piperazin-1-yl)-1H-indazol-3-yl)piperidine-2,6-dione (0.40 g) as a brown, gummy solid. LC-MS (ESI): m/z=439.55 [M+H]⁺.

### Preparation of 3-(7-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione

To a stirred solution of 3-(1-methyl-7-((R)-3-methyl-4-(piperidin-4-ylmethyl)piperazin-1-yl)-1H-indazol-3-yl)piperidine-2,6-dione (0.20 g, 0.46 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.18 mmol)) in DMSO (4 mL), DIPEA (0.6 mL, 3.68 mmol) was added. The reaction was heated at 100° C. for 6 hours. The reaction mixture was quenched with ice-cold water and the resulting precipitate was filtered and isolated to obtain a crude product. The crude product was purified by prep HPLC to afford 3-(7-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (0.12 g) as an off-white solid. 1H NMR (400 MHz, DMSO-d₆): δ 10.90 (s, 1H), 8.77 (s, 1H), 8.26 (s, 1H), 8.18 (d, J=2.00 Hz, 1H), 8.03 (s, 1H), 7.74 (dd, J=2.00, 8.80 Hz, 1H), 7.45 (d, J=9.20 Hz, 1H), 7.39-7.37 (m, 1H), 7.01-6.99 (m, 2H), 6.18 (s, 1H), 4.33-4.32 (m, 5H), 4.24 (s, 3H), 3.57 (s, 3H), 3.17-3.10 (m, 4H), 2.80-2.78 (m, 3H), 2.65-2.64 (m, 3H), 2.34-2.33 (m, 2H), 2.32-2.31 (m, 2H), 1.91-1.90 (m, 1H), 1.60-1.88 (m, 3H), 1.40-1.30 (m, 1H), 0.99-0.97 (m, 5H), δ 0.72-0.70 (m, 1H), 0.52-0.50 (m, 2H), 0.36-0.35 (m, 1H). LC-MS (ESI): m/z=868.60 [M−H]⁻.

### Example 34: 3-(6-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (Compound 443a)

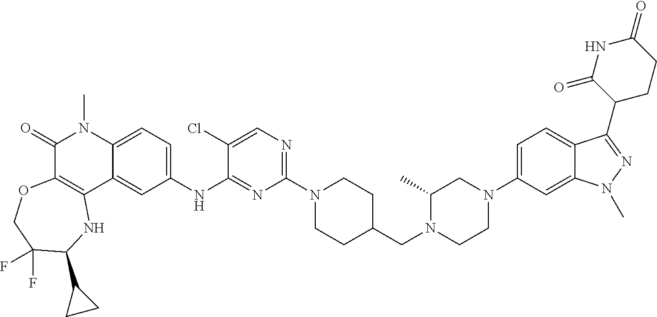 [SMILES: C[C@@H]1CN(c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1]

### Preparation of tert-butyl (R)-4-((4-(3-(2,6-bis(benzyloxy)pyridin-3-yl)-1-methyl-1H-indazol-6-yl)-2-methylpiperazin-1-yl)methyl)piperidine-1-carboxylate

To a stirred solution of 3-(2,6-bis(benzyloxy)pyridin-3-yl)-6-bromo-1-methyl-1H-indazole (0.8 g, 1.59 mmol) and tert-butyl (R)-4-((2-methylpiperazin-1-yl)methyl)piperidine-1-carboxylate (2.6 g, 8.74 mmol) in 1,4-dioxane (16 mL), cesium carbonate (1.55 g, 4.77 mmol) was added. The reaction was degassed for 10 minutes and RuPhos (0.07 g, 0.16 mmol) and RuPhos-Pd G3 (0.07 g, 0.08 mmol) were added. The reaction was stirred at 100° C. for 5 hours. The reaction mixture was cooled to room temperature and then filtered through celite. The filtrate was concentrated under reduced pressure to obtain a crude product. The crude product was purified by flash column chromatography (50% ethyl acetate in petroleum ether) to afford tert-butyl (R)-4-((4-(3-(2,6-bis(benzyloxy)pyridin-3-yl)-1-methyl-1H-indazol-6-yl)-2-methylpiperazin-1-yl)methyl)piperidine-1-carboxylate (0.4 g) as a brown semi-solid. LC-MS (ESI): m/z=717.19 [M+H]⁺.

### Preparation of tert-butyl 4-(((2R)-4-(3-(2,6-dioxopiperidin-3-yl)-1-methyl-1H-indazol-6-yl)-2-methylpiperazin-1-yl)methyl)piperidine-1-carboxylate

To a stirred solution of tert-butyl (R)-4-((4-(3-(2,6-bis(benzyloxy)pyridin-3-yl)-1-methyl-1H-indazol-6-yl)-2-methylpiperazin-1-yl)methyl)piperidine-1-carboxylate (0.40 g, 0.55 mmol) in THF (4 mL), acetic acid (0.4 mL) was added. 20% Pd(OH)₂ on carbon (0.4 g, 100% w/w) was added, and the reaction was put under hydrogen atmosphere (60 psi) and stirred for 5 hours at room temperature. The reaction mixture was diluted with THF and filtered through celite. The celite was washed with excess 20% THF in DCM. The filtrate was concentrated to afford tert-butyl 4-(((2R)-4-(3-(2,6-dioxopiperidin-3-yl)-1-methyl-1H-indazol-6-yl)-2-methylpiperazin-1-yl)methyl)piperidine-1-carboxylate (0.28 g) as a brown semi-solid. LC-MS (ESI): m/z: =539.68 [M+H]⁺.

### Preparation of 3-(1-methyl-6-((R)-3-methyl-4-(piperidin-4-ylmethyl)piperazin-1-yl)-1H-indazol-3-yl)piperidine-2,6-dione (TFA salt)

To a stirred solution of tert-butyl 4-(((2R)-4-(3-(2,6-dioxopiperidin-3-yl)-1-methyl-1H-indazol-6-yl)-2-methylpiperazin-1-yl)methyl)piperidine-1-carboxylate (0.28 g, 0.52 mmol) in DCM (3 mL), TFA (1.4 mL) was added, and the reaction was stirred for 2 hours at room temperature. The reaction mixture was concentrated and triturated with diethyl ether to obtain 3-(1-methyl-6-((R)-3-methyl-4-(piperidin-4-ylmethyl)piperazin-1-yl)-1H-indazol-3-yl)piperidine-2,6-dione (0.35 g) as a TFA salt. LC-MS (ESI): m/z=439.55 [M+H]⁺.

### Preparation of 3-(6-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione

To a stirred solution of 3-(1-methyl-6-((R)-3-methyl-4-(piperidin-4-ylmethyl)piperazin-1-yl)-1H-indazol-3-yl)piperidine-2,6-dione (0.20 g, 0.46 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.18 mmol)) in DMSO (4 mL), DIPEA (0.6 mL, 3.68 mmol) was added. The reaction was heated at 100° C. for 6 hours. The reaction mixture was quenched with ice-cold water and the resulting solid was filtered to obtain a crude product. The crude product was purified by prep HPLC to afford 3-(6-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-methylpiperazin-1-yl)-1-methyl-1H-indazol-3-yl)piperidine-2,6-dione (0.042 g) as an off-white solid. ¹H NMR (400 MHz, DMSO-d₆): δ 10.90 (s, 1H), 8.77 (s, 1H), 8.18 (d, J=2.00 Hz, 1H), 8.03 (s, 1H), 7.74 (dd, J=2.00, 9.20 Hz, 1H), 7.46 (dd, J=9.20, 18.40 Hz, 2H), 6.90 (dd, J=1.60, 9.20 Hz, 1H), 6.82 (s, 1H), 6.18 (bs, 1H), 4.41-4.40 (m, 4H), 4.25 (q, J=4.80 Hz, 1H), 3.89 (s, 3H), 3.56 (s, 3H), 3.50 (d, J=8.80 Hz, 2H), 3.22 (s, 1H), 2.92-2.88 (m, 4H), 2.78-2.67 (m, 3H), 2.45-2.45 (m, 2H), 2.29-2.27 (m, 2H), 2.15-2.11 (m, 3H), 2.16-2.07 (m, 1H), 1.98-1.94 (m, 1H), 1.75-1.62 (m, 2H), 1.36 (s, 1H), 1.04 (d, J=6.00 Hz, 3H), 0.70 (br s, 1H), 0.52-0.50 (m, 2H), 0.36-0.35 (m, 1H). LC-MS (ESI): m/z=868.52 [M−H]⁺.

### Example 35: 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)azetidin-1-yl)-1-methyl-1H-indazol-3-yl)piperidine-2,6-dione (Compound 388a)

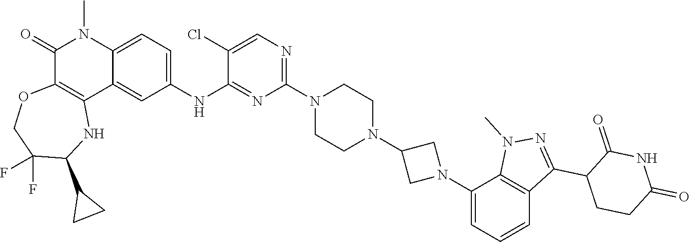 [SMILES: Cn1nc(C2CCC(=O)NC2=O)c2cccc(N3CC(N4CCN(c5ncc(Cl)c(Nc6ccc7c(c6)c6c(c(=O)n7C)OCC(F)(F)[C@H](C7CC7)N6)n5)CC4)C3)c21]

### Preparation of tert-butyl 4-(1-(3-(2,6-bis(benzyloxy)pyridin-3-yl)-1-methyl-1H-indazol-7-yl)azetidin-3-yl)piperazine-1-carboxylate

A stirred solution of 3-(2,6-bis(benzyloxy)pyridin-3-yl)-7-bromo-1-methyl-1H-indazole (0.60 g, 1.20 mmol), tert-butyl 4-(azetidin-3-yl) piperazine-1-carboxylate (0.31 g, 1.31 mmol), and cesium carbonate (1.17 g, 3.59 mmol) in dioxane (12.0 mL) was degassed with argon for 5 minutes.

RuPhos (0.06 g, 0.13 mmol) and RuPhos-Pd G3 (0.06 g, 0.06 mmol) were added, and the reaction was heated at 100° C. for 18 hours. The reaction mixture was quenched with water (5 mL) and extracted into ethyl acetate (3×50 mL). The separated organic layers were dried over anhydrous sodium sulfate, filtered, and concentrated under vacuum to obtain a crude product. The crude product was purified by flash column chromatography (21% ethyl acetate in petroleum ether) to afford tert-butyl 4-(1-(3-(2,6-bis(benzyloxy)pyridin-3-yl)-1-methyl-1H-indazol-7-yl)azetidin-3-yl)piperazine-1-carboxylate (0.60 g) as a pale brown semi-solid. LC-MS (ESI): m/z=661.87 [M+H]⁺.

### Preparation of tert-butyl 4-(1-(3-(2,6-dioxopiperidin-3-yl)-1-methyl-1H-indazol-7-yl) azetidin-3-yl)piperazine-1-carboxylate

To a stirred solution of tert-butyl 4-(1-(3-(2,6-bis(benzyloxy)pyridin-3-yl)-1-methyl-1H-indazol-7-yl)azetidin-3-yl)piperazine-1-carboxylate (0.65 g, 0.98 mmol) in THF (13 mL), acetic acid (0.65 m1), DMF (3.0 ml), and Pd(OH)₂ on carbon (1.38 g, 9.84 mmol) was added. The reaction was put under hydrogen atmosphere (15 psi) and stirred at room temperature for 16 hours. The reaction mixture was diluted with DCM (100 mL) and filtered through celite. The celite was washed with DCM:THF (1:1). The collected filtrate was concentrated to obtain a crude product. The crude product was triturated with diethyl ether (20 mL) and dried under vacuum to afford tert-butyl 4-(1-(3-(2,6-dioxopiperidin-3-yl)-1-methyl-1H-indazol-7-yl) azetidin-3-yl)piperazine-1-carboxylate (0.51 g) as a pale brown semi-solid. LC-MS (ESI): m/z=483.49 [M+H]⁺.

### Preparation of 3-(1-methyl-7-(3-(piperazin-1-yl)azetidin-1-yl)-1H-indazol-3-yl)piperidine-2,6-dione

A stirred solution of tert-butyl 4-(1-(3-(2,6-dioxopiperidin-3-yl)-1-methyl-1H-indazol-7-yl) azetidin-3-yl)piperazine-1-carboxylate (0.51 g, 1.06 mmol) in DCM (6.0 ml) was cooled to 0° C. TFA (6.0 ml) was added and the reaction was allowed to warm to room temperature over 3 hours with stirring. The reaction mixture was concentrated under vacuum to obtain a crude product. The crude product was triturated with petroleum ether (50 mL) and dried under vacuum to afford 3-(1-methyl-7-(3-(piperazin-1-yl)azetidin-1-yl)-1H-indazol-3-yl)piperidine-2,6-dione (0.35 g) as a pale brown semi-solid. LC-MS (ESI): m/z=383.89 [M+H]⁺.

### Preparation of 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)azetidin-1-yl)-1-methyl-1H-indazol-3-yl)piperidine-2,6-dione

To a stirred solution 3-(1-methyl-7-(3-(piperazin-1-yl)azetidin-1-yl)-1H-indazol-3-yl)piperidine-2,6-dione (0.35 g, 0.92 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.25 g, 0.46 mmol) in DMSO (3.5 mL), DIPEA (2.0 mL, 10.98 mmol) was added. The reaction was heated at 100° C. for 16 hours. Solvent was removed under vacuum to obtain a crude product. The crude product was purified by prep HPLC to afford 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)azetidin-1-yl)-1-methyl-1H-indazol-3-yl)piperidine-2,6-dione (0.14 g) as a pale brown solid. ¹H NMR (400 MHz, DMSO-d₆): δ 10.92 (s, 1H), 8.87 (s, 1H), 8.21 (d, J=1.60 Hz, 1H), 8.06 (s, 1H), 7.77 (dd, J=2.00, 9.20 Hz, 1H), 7.43 (d, J=9.20 Hz, 1H), 7.23 (d, J=8.00 Hz, 1H), 7.01-6.97 (m, 1H), 6.74 (d, J=7.20 Hz, 1H), 6.23 (br s, 1H), 4.42-4.29 (m, 3H), 4.20 (s, 3H), 3.99-3.95 (m, 2H), 3.69-3.56 (m, 10H), 2.71-2.68 (m, 2H), 2.41-2.38 (m, 5H), 2.69-2.61 (m, 1H), 1.34-1.32 (m, 1H), 0.72-0.70 (m, 2H), 0.53-0.50 (m, 2H), 0.36-0.35 (m, 1H). LC-MS (ESI): m/z=812.38 [M−H]⁺.

### Example 36: 3-(6-((((R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-3-yl)methyl)amino)-1-methyl-1H-indazol-3-yl)piperidine-2,6-dione (Compound 389a)

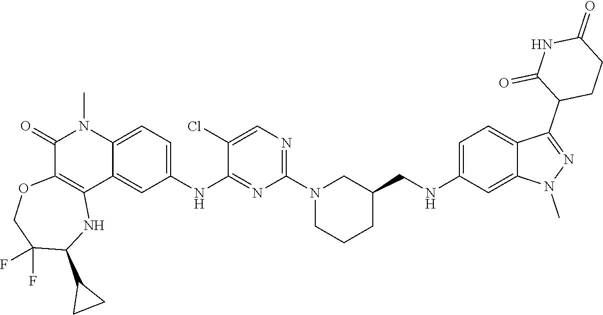 [SMILES: Cn1nc(C2CCC(=O)NC2=O)c2ccc(NC[C@H]3CCCN(c4ncc(Cl)c(Nc5ccc6c(c5)c5c(c(=O)n6C)OCC(F)(F)[C@H](C6CC6)N5)n4)C3)cc21]

### Preparation of tert-butyl (R)-3-(((3-(2,6-bis(benzyloxy)pyridin-3-yl)-1-methyl-1H-indazol-6-yl)amino)methyl)piperidine-1-carboxylate

A stirred solution of 3-(2,6-bis(benzyloxy)pyridin-3-yl)-6-bromo-1-methyl-1H-indazole (1.20 g, 2.40 mmol), tert-butyl (R)-3-(amino methyl) piperidine-1-carboxylate (2.0 g, 9.59 mmol), and cesium carbonate (2.34 g, 7.19 mmol) in dioxane (5.0 mL) was degassed with argon for 5 minutes.

RuPhos (0.11 g, 0.24 mmol) and RuPhos-Pd G3 (0.10 g, 0.12 mmol) were added, and the reaction was heated to 100° C. for 16 hours. The reaction mixture was diluted with ethyl acetate (50 mL) and filtered through celite. The collected filtrate was concentrated under vacuum to obtain a crude product. The crude was purified by column chromatography (25-30% ethyl acetate in petroleum ether) to afford tert-butyl (R)-3-(((3-(2,6-bis(benzyloxy)pyridin-3-yl)-1-methyl-1H-indazol-6-yl)amino)methyl)piperidine-1-carboxylate (0.81 g) as a pale brown semi-solid. LC-MS (ESI): m/z=634.64 [M+H]⁺.

### Preparation of tert-butyl (3R)-3-(((3-(2,6-dioxopiperidin-3-yl)-1-methyl-1H-indazol-6-yl)amino)methyl)piperidine-1-carboxylate

A stirred solution of tert-butyl (R)-3-(((3-(2,6-bis(benzyloxy)pyridin-3-yl)-1-methyl-1H-indazol-6-yl)amino)methyl)piperidine-1-carboxylate (0.81 g, 1.28 mmol) in THF (28.0 mL) was degassed with nitrogen for 5 minutes. 20% Pd(OH)₂ on carbon, powder (0.81 g, 100% w/w) was added, and the reaction was put under hydrogen atmosphere (60 psi) and stirred at room temperature for 4 hours. The reaction mixture was diluted with THF (50 mL) and filtered through celite. The celite was washed with THF:DCM (1:1, 250 mL). The filtrate was concentrated to afford tert-butyl (3R)-3-(((3-(2,6-dioxopiperidin-3-yl)-1-methyl-1H-indazol-6-yl)amino)methyl)piperidine-1-carboxylate (0.75 g) as a pale green semi-solid. LC-MS (ESI): m/z=456.30 [M+H]⁺.

### Preparation of 3-(1-methyl-6-((((S)-piperidin-3-yl)methyl)amino)-1H-indazol-3-yl)piperidine-2,6-dione (TFA salt)

A stirred solution of tert-butyl (3R)-3-(((3-(2,6-dioxopiperidin-3-yl)-1-methyl-1H-indazol-6-yl)amino)methyl)piperidine-1-carboxylate (0.75 g, 1.64 mmol) in DCM (15 mL) was cooled to 0° C. Trifluoroacetic acid (5.0 mL) was added, and the reaction was allowed to warm to room temperature over 2 hours with stirring. The reaction mixture was concentrated under reduced pressure to obtain a crude product. The crude product was washed with n-pentane (20 mL) to afford 3-(1-methyl-6-((((S)-piperidin-3-yl)methyl)amino)-1H-indazol-3-yl)piperidine-2,6-dione (TFA salt) (0.55 g) as a pale brown solid. LC-MS (ESI): m/z=356.21 [M+H]⁺.

### Preparation of 3-(6-((((R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-3-yl)methyl)amino)-1-methyl-1H-indazol-3-yl)piperidine-2,6-dione

To a stirred solution of 3-(1-methyl-6-((((S)-piperidin-3-yl)methyl)amino)-1H-indazol-3-yl)piperidine-2,6-dione (TFA salt) (0.55 g, 1.54 mmol) in DMSO (5.5 mL), DIPEA (4.10 mL, 23.21 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.36 g, 23.21 mmol) was added. The reaction was heated at 80° C. for 16 hours. Solvent was removed under vacuum to obtain a crude product. The crude product was purified by prep HPLC to afford 3-(6-((((R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-3-yl)methyl)amino)-1-methyl-1H-indazol-3-yl)piperidine-2,6-dione (0.108 g) as a brown solid. ¹H NMR (400 MHz, DMSO-d₆): δ 10.83 (s, 1H), 8.76 (s, 1H), 8.03 (s, 1H), 7.82 (s, 1H), 7.38 (d, J=8.80 Hz, 1H), 7.30 (d, J=8.80 Hz, 1H), 6.50 (d, J=8.40 Hz, 1H), 6.31 (s, 1H), 6.17 (s, 1H), 5.92 (s, 1H), 5.76 (s, 1H), 4.30-4.16 (m, 5H), 3.78 (s, 3H), 3.47 (s, 3H), 3.22-3.12 (m, 1H), 3.00-2.97 (m, 3H), 2.71-2.67 (m, 3H), 2.33-2.31 (m, 1H), 2.24-2.22 (m, 1H), 1.91-1.88 (m, 1H), 1.72-1.66 (m, 1H), 1.65-1.59 (m, 1H), 1.42-1.24 (m, 2H), 0.72-0.7 (m, 2H), 0.52-0.52 (m, 2H), 0.34 (d, J=5.20 Hz, 1H).

LC-MS (ESI): m/z=787.48 [M+H]⁺.

### Example 37: 3-(7-(4-((8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,8-diazabicyclo[3.2.1]octan-3-yl)methyl)piperidin-1-yl)-1-methyl-1H-indazol-3-yl)piperidine-2,6-dione (Compound 395a)

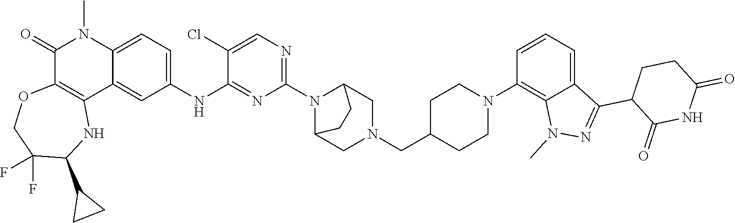 [SMILES: Cn1nc(C2CCC(=O)NC2=O)c2cccc(N3CCC(CN4CC5CCC(C4)N5c4ncc(Cl)c(Nc5ccc6c(c5)c5c(c(=O)n6C)OCC(F)(F)[C@H](C6CC6)N5)n4)CC3)c21]

### Preparation of tert-butyl 3-((1-((benzyloxy)carbonyl)piperidin-4-yl)methyl)-3,8-diazabicyclo[3.2.1]octane-8-carboxylate

To a stirred solution of tert-butyl 3,8-diazabicyclo[3.2.1]octane-8-carboxylate (3.00 g, 14.13 mmol) and benzyl 4-formylpiperidine-1-carboxylate (4.20 g, 16.96 mmol) in DCM (30 mL), acetic acid (0.82 mL, 14.13 mmol) was added. After stirring for 1 hour, sodium triacetoxyborohydride (6.00 g, 28.26 mmol) was added. The reaction was stirred at room temperature for 16 hours. The reaction mixture was quenched with ammonium chloride solution (70 mL) and extracted with DCM (2×80 mL). The combined organic layers were washed with brine solution (50 mL), dried over anhydrous sodium sulfate, filtered, and concentrated under reduced pressure to obtain tert-butyl 3-((1-((benzyloxy)carbonyl)piperidin-4-yl)methyl)-3, 8-diazabicyclo[3.2.1]octane-8-carboxylate (5.81 g) as a pale yellow liquid. LC-MS (ESI): m/z=444.55 [M+H]⁺.

### Preparation of tert-butyl 3-(piperidin-4-ylmethyl)-3,8-diazabicyclo[3.2.1]octane-8-carboxylate

To a solution of tert-butyl 3-((1-((benzyloxy)carbonyl)piperidin-4-yl)methyl)-3,8-diazabicyclo[3.2.1]octane-8-carboxylate (3.00 g, 6.76 mmol) in THF (120 mL), 20% Pd(OH)₂ on carbon (1.80 g, 60% w/w) was added. The reaction was put under hydrogen atmosphere (80 psi) and stirred at room temperature for 16 hours. The reaction mixture was diluted with THF (50 mL) and filtered through celite. The celite was washed with 200 mL of THF:DCM (1:1), and the filtrate was concentrated and dried to afford tert-butyl 3-(piperidin-4-ylmethyl)-3,8-diazabicyclo[3.2.1]octane-8-carboxylate (2.01 g) as a pale yellow gummy compound. LC-MS (ESI): m/z=310.21 [M+H]⁺.

### Preparation of tert-butyl 3-((1-(3-(2,6-bis(benzyloxy)pyridin-3-yl)-1-methyl-1H-indazol-7-yl)piperidin-4-yl)methyl)-3,8-diazabicyclo[3.2.1]octane-8-carboxylate

To a stirred solution of 3-(2,6-bis(benzyloxy)pyridin-3-yl)-7-bromo-1-methyl-1H-indazole (1.00 g, 1.99 mmol) and tert-butyl 3-(piperidin-4-ylmethyl)-3,8-diazabicyclo[3.2.1]octane-8-carboxylate (1.55 g, 4.99 mmol) in 1,4-dioxane (14 mL), cesium carbonate (1.95 g, 5.99 mmol) was added. The reaction was degassed with argon for 15 minutes, Pd-PEPPSI-iHeptCl (0.10 g, 0.10 mmol) was added, and the reaction was stirred at 100° C. for 16 hours. The reaction mixture was diluted with ethyl acetate (50 mL) and filtered through celite. The celite was washed with 10% MeOH in DCM (150 mL). The filtrate was concentrated under vacuum to obtain a crude product.

The crude was purified by flash chromatography (10-15% ethyl acetate in petroleum ether) to afford tert-butyl 3-((1-(3-(2,6-bis(benzyloxy)pyridin-3-yl)-1-methyl-1H-indazol-7-yl)piperidin-4-yl)methyl)-3,8-diazabicyclo[3.2.1]octane-8-carboxylate (0.80 g) as a pale yellow solid. LC-MS (ESI): m/z=729.54 [M+H]⁺.

### Preparation of tert-butyl 3-((1-(3-(2,6-dioxopiperidin-3-yl)-1-methyl-1H-indazol-7-yl)piperidin-4-yl)methyl)-3,8-diazabicyclo[3.2.1]octane-8-carboxylate

To a solution of tert-butyl 3-((1-(3-(2,6-bis(benzyloxy)pyridin-3-yl)-1-methyl-1H-indazol-7-yl)piperidin-4-yl)methyl)-3,8-diazabicyclo[3.2.1]octane-8-carboxylate (0.78 g, 1.07 mmol) in THF (32 mL), 20% Pd(OH)₂ on carbon (0.78 g, 100% w/w) was added. The reaction was put under hydrogen atmosphere (80 psi) and stirred at room temperature for 5 hours. The reaction mixture was diluted with THF (50 mL) and filtered through celite. The celite was washed with THF:DCM (1:1, 150 mL), and the filtrate was concentrated to afford tert-butyl 3-((1-(3-(2,6-dioxopiperidin-3-yl)-1-methyl-1H-indazol-7-yl)piperidin-4-yl)methyl)-3, 8-diazabicyclo[3.2.1]octane-8-carboxylate (0.59 g) as a pale brown solid. LC-MS (ESI): m/z=551.68 [M+H]⁺.

### Preparation of 3-(7-(4-((3,8-diazabicyclo[3.2.1]octan-3-yl)methyl)piperidin-1-yl)-1-methyl-1H-indazol-3-yl)piperidine-2,6-dione

A stirred solution of tert-butyl 3-((1-(3-(2,6-dioxopiperidin-3-yl)-1-methyl-1H-indazol-7-yl)piperidin-4-yl)methyl)-3,8-diazabicyclo[3.2.1]octane-8-carboxylate (0.59 g, 1.07 mmol) in DCM (6 mL) was cooled to 0° C. TFA (3.0 mL) was added, and the reaction was allowed to warm to room temperature over 3 hours with stirring. The reaction mixture was concentrated under reduced pressure and residual solvent was co-distilled with DCM (3×10 mL) to afford 3-(7-(4-((3,8-diazabicyclo[3.2.1]octan-3-yl)methyl)piperidin-1-yl)-1-methyl-1H-indazol-3-yl)piperidine-2,6-dione (0.51 g) as an off-white solid. LC-MS (ESI): m/z=451.29 [M+H]⁺.

### Preparation of 3-(7-(4-((8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,8-diazabicyclo[3.2.1]octan-3-yl)methyl)piperidin-1-yl)-1-methyl-1H-indazol-3-yl)piperidine-2,6-dione

To a stirred solution of 3-(7-(4-((3,8-diazabicyclo[3.2.1]octan-3-yl)methyl)piperidin-1-yl)-1-methyl-1H-indazol-3-yl)piperidine-2,6-dione (0.25 g, 0.55 mmol) and DIPEA (2.0 mL) in DMSO (2.5 mL), (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.22 mmol) was added. The reaction was stirred at 100° C. for 16 hours. Solvent was removed under vacuum to obtain a crude product. The crude product was purified by prep HPLC to afford 3-(7-(4-((8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,8-diazabicyclo[3.2.1]octan-3-yl)methyl)piperidin-1-yl)-1-methyl-1H-indazol-3-yl)piperidine-2,6-dione (0.075 g) as an off-white solid. 1H NMR (400 MHz, DMSO-d6): δ 10.89 (s, 1H), 8.80 (s, 1H), 8.15 (s, 1H), 8.05 (s, 1H), 7.78 (dd, J=1.60, 9.00 Hz, 1H), 7.45 (d, J=9.20 Hz, 1H), 7.36 (dd, J=2.40, 6.80 Hz, 1H), 7.03-6.98 (m, 2H), 6.15 (br s, 1H), 4.45-4.31 (m, 5H), 4.23 (s, 3H), 3.57 (s, 3H), 3.24-3.23 (m, 3H), 2.67-2.62 (m, 6H), 2.38-2.15 (m, 6H), 1.84-1.77 (m, 7H), 1.43-1.24 (m, 3H), 0.72-0.36 (m, 4H). LC-MS (ESI): m/z=882.55 [M+H]⁺.

### Example 38: 3-(6-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperazin-1-yl)methyl)piperidin-1-yl)-1-methyl-1H-indazol-3-yl)piperidine-2,6-dione (Compound 382a)

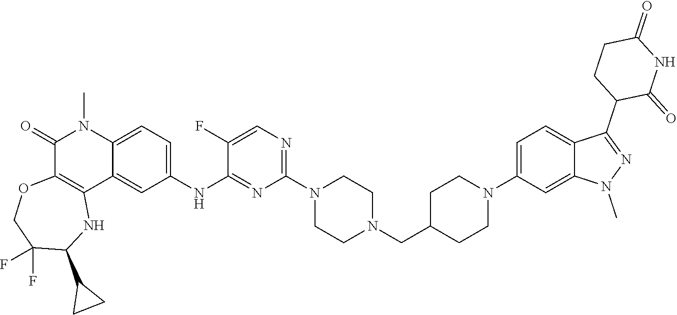 [SMILES: Cn1nc(C2CCC(=O)NC2=O)c2ccc(N3CCC(CN4CCN(c5ncc(F)c(Nc6ccc7c(c6)c6c(c(=O)n7C)OCC(F)(F)[C@H](C7CC7)N6)n5)CC4)CC3)cc21]

### Preparation of 3-(6-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperazin-1-yl)methyl)piperidin-1-yl)-1-methyl-1H-indazol-3-yl)piperidine-2,6-dione

A mixture of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (50.0 mg, 111 μmol), 3-(1-methyl-6-(4-(piperazin-1-ylmethyl)piperidin-1-yl)-1H-indazol-3-yl)piperidine-2,6-dione hydrochloride (61.2 mg, 133 μmol), and CsF (50.4 mg 332 μmol) in DMSO (1.00 mL) was warmed to 80° C. for 20 hours, then treated with DIEA (42.9 mg, 57.8 μL, 332 μmol) and stirred at 80° C. for 4 hours. The reaction mixture was then warmed to 110° C. for 18 hours, cooled to room temperature, filtered, and purified by prep HPLC (37-41% MeCN/water [w/0.1% formic acid]) to afford 3-(6-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperazin-1-yl)methyl)piperidin-1-yl)-1-methyl-1H-indazol-3-yl)piperidine-2,6-dione (32.0 mg, 38.1 μmol) as a white solid. 1H NMR (400 MHz, DMSO-d₆): δ 10.83 (s, 1H), 9.33 (s, 1H), 8.27 (d, J=2.3 Hz, 1H), 8.02 (d, J=3.7 Hz, 1H), 7.73 (dd, J=9.1, 2.2 Hz, 1H), 7.46 (dd, J=13.4, 9.1 Hz, 2H), 6.90 (d, J=9.5 Hz, 1H), 6.82 (s, 1H), 6.06 (s, 1H), 4.52-4.30 (m, 2H), 4.24 (dd, J=9.2, 5.1 Hz, 1H), 3.88 (s, 3H), 3.78 (d, J=12.0 Hz, 2H), 3.59 (s, 5H), 3.55 (s, 3H), 2.78-2.56 (m, 3H), 2.37 (s, 3H), 2.18 (d, J=7.0 Hz, 3H), 1.81 (d, J=12.5 Hz, 2H), 1.72 (s, 2H), 1.28 (s, 3H), 0.71 (t, J=6.8 Hz, 1H), 0.53 (t, J=6.2 Hz, 2H), 0.37 (d, J=5.3 Hz, 1H). LC-MS (ESI): m/z=840.5 [M+H]⁺.

### Example 39: 3-(7-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperazin-1-yl)methyl)piperidin-1-yl)-1-methyl-1H-indazol-3-yl)piperidine-2,6-dione (Compound 383a)

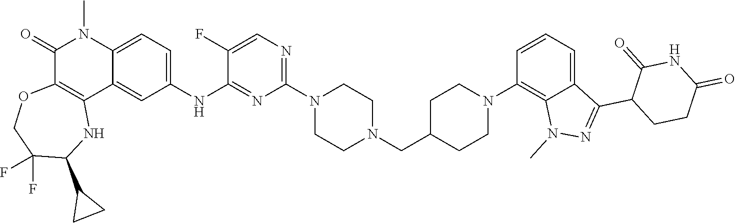 [SMILES: Cn1nc(C2CCC(=O)NC2=O)c2cccc(N3CCC(CN4CCN(c5ncc(F)c(Nc6ccc7c(c6)c6c(c(=O)n7C)OCC(F)(F)[C@H](C7CC7)N6)n5)CC4)CC3)c21]

### Preparation of 3-(7-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperazin-1-yl)methyl)piperidin-1-yl)-1-methyl-1H-indazol-3-yl)piperidine-2,6-dione

A mixture of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (50.0 mg, 111 μmol), 3-(1-methyl-7-(4-(piperazin-1-ylmethyl)piperidin-1-yl)-1H-indazol-3-yl)piperidine-2,6-dione hydrochloride (61.2 mg, 133 μmol), and CsF (50.4 mg, 332 μmol) in DMSO (1.00 mL) was warmed to 80° C. for 20 hours, then treated with DIEA (42.9 mg, 57.8 μL, 3.00 equiv., 332 μmol) and stirred at 80° C. for 4 hours, then warmed to 110° C. for 18 hours. The reaction mixture was cooled to room temperature, filtered, and purified by prep HPLC (37-40% MeCN/water [w/0.1% formic acid]) to afford 3-(7-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperazin-1-yl)methyl)piperidin-1-yl)-1-methyl-1H-indazol-3-yl)piperidine-2,6-dione (39.8 mg, 47.3 μmol) as an off-white solid. ¹H NMR (400 MHz, DMSO-d₆): δ 10.87 (s, 1H), 9.33 (s, 1H), 8.27 (s, 1H), 8.02 (d, J=3.7 Hz, 1H), 7.73 (d, J=10.6 Hz, 1H), 7.44 (d, J=9.2 Hz, 1H), 7.36 (d, J=8.8 Hz, 1H), 7.01 (d, J=7.1 Hz, 2H), 6.06 (s, 1H), 4.54-4.34 (m, 2H), 4.33-4.28 (m, 1H), 4.24 (s, 3H), 3.63-3.58 (m, 5H), 3.56 (s, 2H), 2.73-2.59 (m, 4H), 2.39 (br s, 4H), 2.24 (s, 1H), 1.85 (s, 1H), 1.79-1.58 (m, 4H), 1.43-1.26 (m, 4H), 0.70 (t, J=6.8 Hz, 1H), 0.52 (s, 2H), 0.36 (d, J=5.3 Hz, 1H). LC-MS (ESI): m/z=840.5 [M+H]⁺.

### Example 40: 3-(7-(4-((4-(5-bromo-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (Compound 384a)

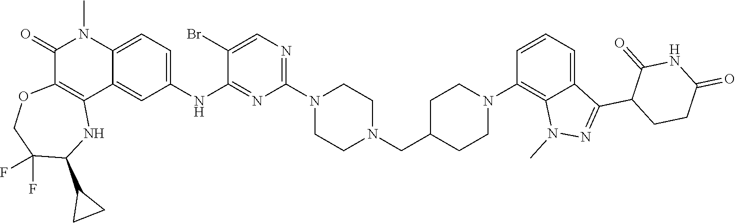 [SMILES: Cn1nc(C2CCC(=O)NC2=O)c2cccc(N3CCC(CN4CCN(c5ncc(Br)c(Nc6ccc7c(c6)c6c(c(=O)n7C)OCC(F)(F)[C@H](C7CC7)N6)n5)CC4)CC3)c21]

### Preparation of 3-(7-(4-((4-(5-bromo-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione

A mixture of (S)-10-((5-bromo-2-chloropyrimidin-4-yl)amino)-2-cyclopropyl-3,3-difluoro-7-methyl-1,2,3,4-tetrahydro-[1,4]oxazepino[2,3-c]quinolin-6(7H)-one (52.8 mg, 103 μmol), 3-(1-methyl-7-(4-(piperazin-1-ylmethyl)piperidin-1-yl)-1H-indazol-3-yl)piperidine-2,6-dione hydrochloride (57.0 mg, 124 μmol), and CsF (46.9 mg, 309 μmol) in DMSO (1.00 mL) was warmed to 80° C. for 4 hours, then cooled to room temperature. The reaction mixture was filtered and purified by prep HPLC (10-63-100% MeCN/water w/0.1% formic acid) to afford 3-(7-(4-((4-(5-bromo-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (34.1 mg, 37.8 μmol). ¹H NMR (400 MHz, DMSO-d₆): δ 10.80 (s, 1H), 8.51 (s, 1H), 8.13 (s, 1H), 8.05 (s, 1H), 7.63 (d, J=8.9 Hz, 1H), 7.37 (d, J=9.1 Hz, 1H), 7.30 (d, J=7.5 Hz, 1H), 6.94 (d, J=7.0 Hz, 2H), 6.17 (s, 1H), 4.34 (s, 2H), 4.26 (dd, J=9.6, 5.2 Hz, 1H), 4.17 (s, 3H), 3.50 (s, 3H), 3.21-3.10 (m, 8H), 2.62-2.51 (m, 4H), 2.34-2.27 (m, 4H), 2.12 (dd, J=16.2, 10.6 Hz, 2H), 1.86-1.72 (m, 3H), 1.69-1.56 (m, 1H), 1.35-1.19 (m, 2H), 0.72-0.58 (m, 1H), 0.45 (d, J=6.5 Hz, 2H), 0.34-0.22 (m, 1H). LC-MS (ESI): m/z=900.5 [M+H]⁺.

### Example 41: 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperazin-1-yl)methyl)piperidin-1-yl)-1-methyl-1H-indazol-3-yl)piperidine-2,6-dione (Compound 385a)

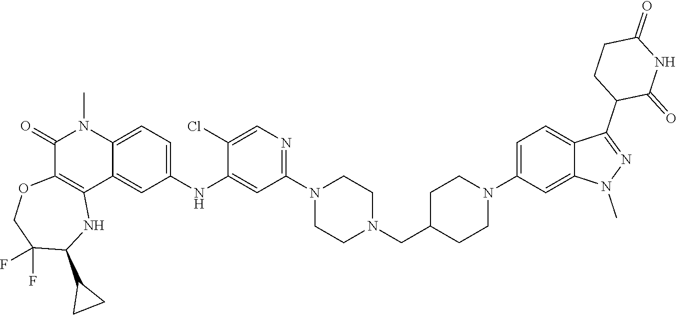 [SMILES: Cn1nc(C2CCC(=O)NC2=O)c2ccc(N3CCC(CN4CCN(c5cc(Nc6ccc7c(c6)c6c(c(=O)n7C)OCC(F)(F)[C@H](C7CC7)N6)c(Cl)cn5)CC4)CC3)cc21]

### Preparation of 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperazin-1-yl)methyl)piperidin-1-yl)-1-methyl-1H-indazol-3-yl)piperidine-2,6-dione

A mixture of (S)-10-((5-chloro-2-fluoropyridin-4-yl)amino)-2-cyclopropyl-3,3-difluoro-7-methyl-1,2,3,4-tetrahydro-[1,4]oxazepino [2,3-c]quinolin-6(7H)-one (50.0 mg, 111 μmol), 3-(1-methyl-6-(4-(piperazin-1-ylmethyl)piperidin-1-yl)-1H-indazol-3-yl)piperidine-2,6-dione hydrochloride (61.4 mg, 133 μmol), and CsF (50.5 mg, 333 μmol) in DMSO (1.00 mL) was warmed to 80° C. for 20 hours. The reaction mixture was then treated with DIEA (43.0 mg, 58.0 μL, 333 μmol), warmed to 80° C. for 4 hours, then warmed to 110° C. for 4 days. The reaction mixture was cooled to room temperature, filtered, and purified by prep HPLC (10-100% MeCN/water w/0.1% formic acid) to afford 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperazin-1-yl)methyl)piperidin-1-yl)-1-methyl-1H-indazol-3-yl)piperidine-2,6-dione (25.8 mg, 30 μmol) as an off-white solid. 1H NMR (400 MHz, DMSO-d₆): δ 10.83 (s, 1H), 8.06 (d, J=8.7 Hz, 2H), 7.94 (s, 1H), 7.52-7.42 (m, 3H), 6.89 (d, J=9.0 Hz, 1H), 6.81 (s, 1H), 6.42 (s, 1H), 6.09 (s, 1H), 4.51-4.32 (m, 2H), 4.24 (dd, J=9.0, 5.2 Hz, 1H), 3.87 (s, 3H), 3.76 (d, J=12.2 Hz, 3H), 3.57 (s, 4H), 2.72 (d, J=11.9 Hz, 1H), 2.60 (s, 2H), 2.40-2.35 (m, 5H), 2.31-2.21 (m, 3H), 2.16 (s, 3H), 1.86-1.75 (m, 2H), 1.72-1.60 (m, 1H), 1.35-1.18 (m, 4H), 0.77-0.064 (m, 1H), 0.55-0.44 (m, 2H), 0.41-0.22 (m, 1H). LC-MS (ESI): m/z=428.4 [M+2H]²⁺.

### Example 42: 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperazin-1-yl)methyl)piperidin-1-yl)-1-methyl-1H-indazol-3-yl)piperidine-2,6-dione (Compound 386a)

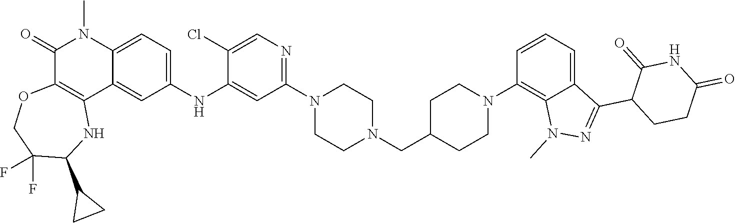 [SMILES: Cn1nc(C2CCC(=O)NC2=O)c2cccc(N3CCC(CN4CCN(c5cc(Nc6ccc7c(c6)c6c(c(=O)n7C)OCC(F)(F)[C@H](C7CC7)N6)c(Cl)cn5)CC4)CC3)c21]

### Preparation of 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperazin-1-yl)methyl)piperidin-1-yl)-1-methyl-1H-indazol-3-yl)piperidine-2,6-dione

A mixture of (S)-10-((5-chloro-2-fluoropyridin-4-yl)amino)-2-cyclopropyl-3,3-difluoro-7-methyl-1,2,3,4-tetrahydro-[1,4]oxazepino [2,3-c]quinolin-6(7H)-one (50.0 mg, 111 μmol), 3-(1-methyl-7-(4-(piperazin-1-ylmethyl)piperidin-1-yl)-1H-indazol-3-yl)piperidine-2,6-dione hydrochloride (61.4 mg, 133 μmol), and CsF (50.5 mg, 333 μmol) in DMSO (1.00 mL) was warmed to 80° C. for 20 hours, then treated with DIEA (43.0 mg, 58.0 μL, 3.00 equiv., 333 μmol) and stirred at 80° C. for an additional 4 hours. The reaction mixture was then warmed to 110° C. for 4 days, cooled to room temperature, filtered, and purified by prep HPLC (10-62-100% MeCN/water w/0.1% formic acid) to afford 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperazin-1-yl)methyl)piperidin-1-yl)-1-methyl-1H-indazol-3-yl)piperidine-2,6-dione (36.4 mg, 42 μmol) as a white solid. 1H NMR (400 MHz, DMSO-d₆): δ 10.87 (s, 1H), 8.20 (s, 1H), 8.07 (s, 1H), 8.00 (s, 1H), 7.60-7.43 (m, 2H), 7.39 (d, J=8.7 Hz, 1H), 7.02 (d, J=6.3 Hz, 2H), 6.39 (s, 1H), 6.15 (s, 1H), 4.52-4.40 (m, 1H), 4.33 (dd, J=9.8, 5.0 Hz, 1H), 4.24 (s, 3H), 4.07 (s, 2H), 3.58 (s, 3H), 3.52 (s, 2H), 3.27-3.18 (m, 2H), 3.10 (d, J=32.0 Hz, 10H), 2.71-2.58 (m, 2H), 2.39-2.25 (m, 2H), 2.21-1.78 (m, 3H), 1.52 (s, 2H), 1.32 (s, 1H), 0.72 (s, 1H), 0.51 (s, 2H), 0.31 (s, 1H). LC-MS (ESI): m/z=855.4 [M+H]⁺.

### Example 43: 3-(3-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)phenyl)piperidine-2,6-dione (Compound 387a)

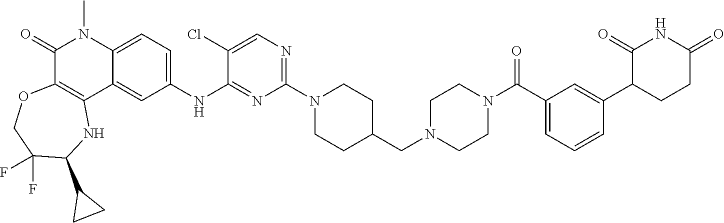 [SMILES: Cn1c(=O)c2c(c3cc(Nc4nc(N5CCC(CN6CCN(C(=O)c7cccc(C8CCC(=O)NC8=O)c7)CC6)CC5)ncc4Cl)ccc31)N[C@@H](C1CC1)C(F)(F)CO2]

### Preparation of tert-butyl 4-((4-(3-(2,6-dioxopiperidin-3-yl)benzoyl)piperazin-1-yl)methyl)piperidine-1-carboxylate

A mixture of tert-butyl 4-(piperazin-1-ylmethyl)piperidine-1-carboxylate (150 mg, 529 μmol), 3-(2,6-dioxopiperidin-3-yl)benzoic acid (148 mg, 635 μmol), and N-methylimidazole (88.6 μL, 1.11 mmol) in MeCN (3.00 mL) was treated with N,N,N′,N′-tetramethylchloroformamidinium hexafluorophosphate (178 mg, 1.20 equiv., 635 μmol) and MeCN (1.00 mL). The reaction mixture was allowed to stir for 1 hour, then diluted with DMSO, concentrated under vacuum to remove acetonitrile, filtered, and purified by reverse phase column chromatography (20-50% MeCN/water [w/0.1% formic acid]) to afford tert-butyl 4-((4-(3-(2,6-dioxopiperidin-3-yl)benzoyl)piperazin-1-yl)methyl)piperidine-1-carboxylate (127 mg, 255 μmol). LC-MS (ESI): m/z=499.6 [M+H]⁺.

### Preparation of 3-(3-(4-(piperidin-4-ylmethyl)piperazine-1-carbonyl)phenyl)piperidine-2,6-dione dihydrochloride

A mixture of tert-butyl 4-((4-(3-(2,6-dioxopiperidin-3-yl)benzoyl)piperazin-1-yl)methyl)piperidine-1-carboxylate (123.5 mg, 247.7 μmol) and DCM (1.00 mL) was treated with HCl (4.0 M in dioxane) (1.00 mL, 4.00 molar, 4.00 mmol) and stirred at room temperature for 21 hours. The volatiles were removed under reduced pressure. The residue was suspended in DCM and concentrated twice more under reduced pressure to afford 3-(3-(4-(piperidin-4-ylmethyl)piperazine-1-carbonyl)phenyl)piperidine-2,6-dione dihydrochloride (116 mg, 246 μmol) as a beige solid. LC-MS (ESI): m/z=399.3 [M+H]⁺.

### Preparation of 3-(3-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)phenyl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (40.0 mg, 85.4 μmol), 3-(3-(4-(piperidin-4-ylmethyl)piperazine-1-carbonyl)phenyl)piperidine-2,6-dione (44.3 mg, 111 μmol), and CsF (38.9 mg, 256 μmol) in DMSO (854 μL) was warmed to 75° C. for 3 hours. The reaction mixture was then treated with DIEA (44.2 mg, 59.5 μL, 342 μmol) and stirred at 75° C. for 17 hours. The reaction mixture was cooled to room temperature, filtered, and purified by prep HPLC (10-45%-100% MeCN/water w/0.1% formic acid) to afford 3-(3-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)phenyl)piperidine-2,6-dione (35.6 mg, 42 μmol) as a pale beige solid. 1H NMR (400 MHz, DMSO-d₆): δ 10.85 (s, 1H), 8.76 (s, 1H), 8.17 (d, J=2.3 Hz, 1H), 8.02 (s, 1H), 7.76-7.67 (m, 1H), 7.48-7.35 (m, 2H), 7.29 (dd, J=14.1, 7.6 Hz, 2H), 7.24 (s, 1H), 6.16 (s, 1H), 4.52-4.28 (m, 4H), 3.92 (dd, J=11.9, 4.9 Hz, 1H), 3.59 (s, 1H), 3.56 (s, 3H), 2.76 (t, J=12.4 Hz, 2H), 2.68-2.64 (m, 1H), 2.43-2.21 (m, 10H), 2.14 (d, J=6.7 Hz, 2H), 2.06 (d, J=4.4 Hz, 1H), 1.70 (d, J=13.1 Hz, 3H), 1.33 (s, 1H), 1.04-0.88 (m, 2H), 0.70 (d, J=7.4 Hz, 1H), 0.51 (t, J=6.0 Hz, 2H), 0.34 (d, J=5.4 Hz, 1H).

LC-MS (ESI): m/z=830.5 [M+H]⁺.

### Example 44: 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)oxy)-1-methyl-1H-indazol-3-yl)piperidine-2,6-dione (Compound 449a)

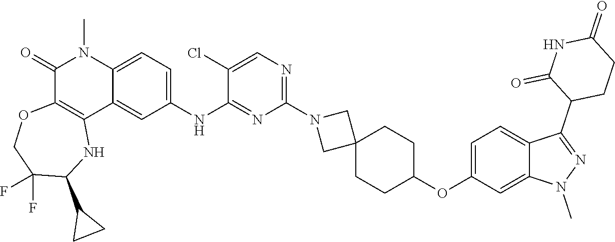 [SMILES: Cn1nc(C2CCC(=O)NC2=O)c2ccc(OC3CCC4(CC3)CN(c3ncc(Cl)c(Nc5ccc6c(c5)c5c(c(=O)n6C)OCC(F)(F)[C@H](C6CC6)N5)n3)C4)cc21]

### Preparation of 3-(2,6-bis(benzyloxy)pyridin-3-yl)-1-methyl-1H-indazol-6-ol

To a stirred solution of 3-(2,6-bis(benzyloxy)pyridin-3-yl)-6-bromo-1-methyl-1H-indazole (1.0 g, 2.00 mmol) in 1,4-dioxane (10.0 mL) and water (10.0 mL), potassium hydroxide (0.33 g, 5.99 mmol) was added. The reaction was purged with nitrogen for 15 minutes, tert-Butyl XPhos (0.10 g, 0.20 mmol) and Pd₂dba₃ (0.18 g, 0.20 mmol) were added, and the reaction was stirred at 100° C. 16 hours. The reaction mixture diluted with water (100 mL) and extracted into ethyl acetate (2×100 mL). The combined organic layers were washed with brine solution, dried over anhydrous sodium sulfate, filtered, and concentrated to obtain a crude product. The crude product was purified by flash column chromatography (35% ethyl acetate in petroleum ether) to afford 3-(2,6-bis(benzyloxy)pyridin-3-yl)-1-methyl-1H-indazol-6-ol (0.7 g) as a yellow solid. LC-MS (ESI): m/z=438.46 [M+H]⁺.

### Preparation of tert-butyl 7-((methylsulfonyl)oxy)-2-azaspiro[3.5]nonane-2-carboxylate

A stirred solution of tert-butyl 4-(4-hydroxybutyl)piperidine-1-carboxylate (0.7 g, 2.9 mmol) and TEA (0.83 mL, 5.8 mmol) in DCM (14.0 mL) was cooled to 0° C. Methanesulfonyl chloride (0.39 g, 3.48 mmol) was added and the reaction was allowed to warm to room temperature over 4 hours with stirring. The reaction mixture was diluted with water (100 mL) and extracted into DCM (2×100 mL). The combined organic layers were washed with brine solution, dried over anhydrous sodium sulfate, filtered, and concentrated under reduced pressure to isolate a crude product. The crude product was purified by trituration using diethyl ether to afford tert-butyl 7-((methylsulfonyl)oxy)-2-azaspiro[3.5]nonane-2-carboxylate (0.7 g) as a yellow semi-solid. ¹H-NMR (400 MHz, CDCl₃): δ 4.72 (m, 1H), 3.61 (m, 2H), 3.01 (m, 2H), 3.01 (s, 3H), 1.93 (m, 4H), 1.65 (m, 4H), 1.44 (s, 9H).

### Preparation of tert-butyl 7-((3-(2,6-bis(benzyloxy)pyridin-3-yl)-1-methyl-1H-indazol-6-yl)oxy)-2-azaspiro[3.5]nonane-2-carboxylate

To a stirred solution of 3-(2,6-bis(benzyloxy)pyridin-3-yl)-1-methyl-1H-indazol-6-ol (0.7 g, 1.60 mmol) and tert-butyl 7-((methylsulfonyl)oxy)-2-azaspiro[3.5]nonane-2-carboxylate (0.61 g, 1.92 mmol) in DMF (14.0 mL) at 0° C., Cs₂CO₃ (1.56 g, 4.80 mmol) was added. The reaction was then stirred at 100° C. for 16 hours. The reaction mixture was diluted with water (70 mL) and extracted into ethyl acetate (150 mL). The combined organic layers were washed with brine solution, dried over anhydrous sodium sulfate, filtered, and concentrated to obtain a crude product. The crude product was purified by flash column chromatography (20% ethyl acetate in petroleum ether) to afford tert-butyl 7-((3-(2,6-bis(benzyloxy)pyridin-3-yl)-1-methyl-1H-indazol-6-yl)oxy)-2-azaspiro[3.5]nonane-2-carboxylate (0.8 g) as a yellow solid. LC-MS (ESI): m/z=661.73 [M+H]⁺.

### Preparation of tert-butyl 7-((3-(2,6-dioxopiperidin-3-yl)-1-methyl-1H-indazol-6-yl)oxy)-2-azaspiro[3.5]nonane-2-carboxylate

To a stirred solution of tert-butyl 7-((3-(2,6-bis(benzyloxy)pyridin-3-yl)-1-methyl-1H-indazol-6-yl)oxy)-2-azaspiro[3.5]nonane-2-carboxylate (0.7 g, 1.06 mmol) in THF (21 mL), 20% Pd(OH)₂ on carbon (1.4 g) was added. The reaction was put under hydrogen atmosphere (20 psi) at room temperature and stirred for 4 hours. The reaction mixture was diluted with ethyl acetate (100 mL) and filtered through celite. The celite was washed with ethyl acetate (2×50 mL). The collected filtrate was concentrated under reduced pressure to obtain a crude product. The crude product was triturated with diethyl ether to afford tert-butyl 7-((3-(2,6-dioxopiperidin-3-yl)-1-methyl-1H-indazol-6-yl)oxy)-2-azaspiro[3.5]nonane-2-carboxylate (0.5 g) as an off-white solid. LC-MS (ESI): m/z=483.53 [M+Na]⁺.

### Preparation of 3-(6-((2-azaspiro[3.5]nonan-7-yl)oxy)-1-methyl-1H-indazol-3-yl)piperidine-2,6-dione

To a stirred solution of tert-butyl 7-((3-(2,6-dioxopiperidin-3-yl)-1-methyl-1H-indazol-6-yl)oxy)-2-azaspiro[3.5]nonane-2-carboxylate (0.5 g, 1.09 mmol) in DCM (5 mL) was added TFA (2.5 mL). After stirring at room temperature for 3 hours, the reaction mixture was concentrated under reduced pressure to obtain a crude product. The crude product was triturated using diethyl ether to afford 3-(6-((2-azaspiro[3.5]nonan-7-yl)oxy)-1-methyl-1H-indazol-3-yl)piperidine-2,6-dione (0.4 g (TFA salt)). LC-MS (ESI): m/z=383.51 [M+H]⁺.

### Preparation of 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)oxy)-1-methyl-1H-indazol-3-yl)piperidine-2,6-dione

To a stirred solution of 3-(6-((2-azaspiro[3.5]nonan-7-yl)oxy)-1-methyl-1H-indazol-3-yl)piperidine-2,6-dione (TFA salt) (0.2 g, 0.41 mmol) in DMSO (4 mL), (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.07 g, 0.16 mmol) and DIPEA (0.38 mL, 2.0 mmol) were added. The reaction was heated at 100° C. for 4 hours. Solvent was removed under vacuum to obtain a crude product. The crude product was purified by prep HPLC to afford 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)oxy)-1-methyl-1H-indazol-3-yl)piperidine-2,6-dione (58 mg) as an off-white solid. 1H-NMR (400 MHz, DMSO-d₆): δ 10.85 (s, 1H), 8.74 (s, 1H), 8.34 (s, 1H), 8.24 (s, 1H), 8.02 (s, 1H), 7.86-7.84 (m, 1H), 7.56-7.53 (m, 1H), 7.45-7.43 (m, 1H), 7.07 (s, 1H), 6.73-6.71 (m, 1H), 6.13 (s, 1H), 4.46-4.36 (m, 3H). 4.30-4.28 (m,1H), 3.91 (s, 3H), 3.70-3.68 (m, 4H), 3.57 (s, 3H), 3.33 (s, 1H), 2.67-2.59 (m, 2H), 2.17-2.13 (m, 1H), 1.91-1.88 (m, 3H), 1.66-1.56 (m, 4H), 1.32 (s, 1H), 0.73-0.71 (d, J=8.0 Hz, 1H), 0.54-0.51 (t, J=6.0 Hz, 2H), 0.37-0.36 (d, J=4.0 Hz, 1H). LC-MS (ESI): m/z=814.47 [M+H]⁺.

### Example 45: 3-(6-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.3]heptan-2-yl)methyl)piperidin-1-yl)-1-methyl-1H-indazol-3-yl)piperidine-2,6-dione (Compound 438a)

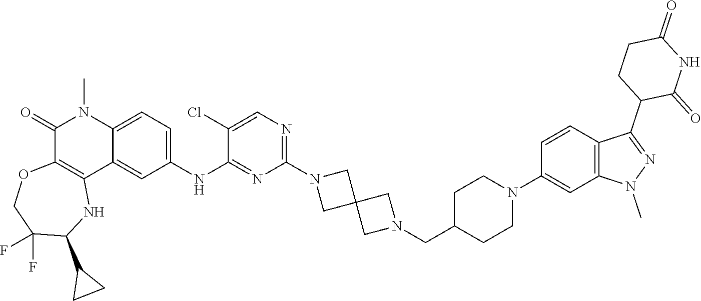 [SMILES: Cn1nc(C2CCC(=O)NC2=O)c2ccc(N3CCC(CN4CC5(C4)CN(c4ncc(Cl)c(Nc6ccc7c(c6)c6c(c(=O)n7C)OCC(F)(F)[C@H](C7CC7)N6)n4)C5)CC3)cc21]

### Preparation of tert-butyl 6-((1-((benzyloxy)carbonyl)piperidin-4-yl)methyl)-2,6-diazaspiro[3.3]heptane-2-carboxylate

tert-Butyl 2,6-diazaspiro[3.3]heptane-2-carboxylate (1 g, 5.04 mmol) and benzyl 4-formylpiperidine-1-carboxylate (1.49 g, 6.05 mmol) were dissolved in a mixture of THF:MeOH (1:1) (20 mL), and stirred at room temperature for 1 hour under N2 atmosphere. After 1 hour, sodium cyanoborohydride (0.63 g, 10.0 mmol) was added at 0° C. The reaction was allowed to warm to room temperature over 16 hours with stirring. The reaction mixture was concentrated under reduced pressure. The crude reaction was diluted with water (100 mL) and extracted with ethyl acetate (3×100 mL). The combined organic layers were concentrated under reduced pressure to afford a crude product. The crude product was purified by flash column chromatography (25% ethyl acetate in petroleum ether) to afford tert-butyl 6-((1-((benzyloxy)carbonyl)piperidin-4-yl)methyl)-2,6-diazaspiro[3.3]heptane-2-carboxylate (0.9 g).

LC-MS (ESI): m/z=430.56 [M+H]⁺.

### Preparation of tert-butyl 6-(piperidin-4-ylmethyl)-2,6-diazaspiro[3.3]heptane-2-carboxylate

A stirred solution of tert-butyl 6-((1-((benzyloxy)carbonyl)piperidin-4-yl)methyl)-2,6-diazaspiro[3.3]heptane-2-carboxylate (0.8 g, 1.86 mmol) in ethanol (16 mL) was purged with N2 for 5 minutes. Palladium on activated carbon (10%) (0.80 g, 1.86 mmol) was added at room temperature. The reaction mixture was put under H₂ atmosphere (100 psi) and stirred at room temperature for 16 hours. The reaction mixture was filtered through celite and the celite was washed with ethyl acetate (100 mL). The filtrate was concentrated under reduced pressure to afford tert-butyl 6-(piperidin-4-ylmethyl)-2,6-diazaspiro[3.3]heptane-2-carboxylate (0.50 g). LC-MS (ESI): m/z=296.21 [M+H]⁺.

### Preparation of tert-butyl 6-((1-(3-(2,6-bis(benzyloxy)pyridin-3-yl)-1-methyl-1H-indazol-6-yl)piperidin-4-yl)methyl)-2,6-diazaspiro[3.3]heptane-2-carboxylate

To a stirred solution of tert-butyl 6-(piperidin-4-ylmethyl)-2,6-diazaspiro[3.3]heptane-2-carboxylate (0.5 g, 1.69 mmol) and 3-(2,6-bis(benzyloxy)pyridin-3-yl)-6-bromo-1-methyl-1H-indazole (0.42 g, 0.84 mmol) in 1,4 dioxane (5 mL), sodium tert-butoxide (0.49 g, 5.07 mmol) was added. The reaction was purged with N2 for 5 minutes, RuPhos-Pd G3 (0.14 g, 0.16 mmol) was added. The reaction was sparged again with N₂ for 5 minutes and then stirred and heated at 100° C. for 16 hours. The reaction mixture was quenched with ice cold water (50 mL) and extracted with ethyl acetate (2×100 mL). The separated organic layers were washed with brine solution (50 mL), dried over anhydrous sodium sulfate, filtered, and concentrated to afford a crude product. The crude product was purified by flash column chromatography (50-60% ethyl acetate in petroleum ether) to afford tert-butyl 6-((1-(3-(2,6-bis(benzyloxy)pyridin-3-yl)-1-methyl-1H-indazol-6-yl)piperidin-4-yl)methyl)-2,6-diazaspiro[3.3]heptane-2-carboxylate (0.75 g). LC-MS (ESI): m/z=715.77 [M+H]⁺.

### Preparation of tert-butyl 6-((1-(3-(2,6-dioxopiperidin-3-yl)-1-methyl-1H-indazol-6-yl)piperidin-4-yl)methyl)-2,6-diazaspiro[3.3]heptane-2-carboxylate

A stirred solution of tert-butyl 6-((1-(3-(2,6-bis(benzyloxy)pyridin-3-yl)-1-methyl-1H-indazol-6-yl)piperidin-4-yl)methyl)-2,6-diazaspiro[3.3]heptane-2-carboxylate (0.6 g, 0.83 mmol) and acetic acid (0.06 mL) in THF (18 mL) was degassed with nitrogen for 5 minutes and then 20% Pd(OH)₂ on carbon, 50% w/w (0.60 g) was added to the reaction. The reaction mixture was put under H₂ atmosphere (15 psi) and stirred at room temperature for 16 hours. The reaction mixture was diluted with ethyl acetate (50 mL) and filtered through celite, and the filtrate was concentrated to obtain a crude product. The crude product was purified by trituration with diethyl ether (2×20 mL) to afford tert-butyl 6-((1-(3-(2,6-dioxopiperidin-3-yl)-1-methyl-1H-indazol-6-yl)piperidin-4-yl)methyl)-2,6-diazaspiro[3.3]heptane-2-carboxylate as (0.41 g). LC-MS (ESI): m/z=537.66 [M+H]⁺.

### Preparation of 3-(6-(4-((2,6-diazaspiro[3.3]heptan-2-yl)methyl)piperidin-1-yl)-1-methyl-1H-indazol-3-yl)piperidine-2,6-dione

To a stirred solution of tert-butyl 6-((1-(3-(2,6-dioxopiperidin-3-yl)-1-methyl-1H-indazol-6-yl)piperidin-4-yl)methyl)-2,6-diazaspiro[3.3]heptane-2-carboxylate (0.4 g, 0.74 mmol) in DCM (4 mL), 4M HCl in dioxane (2 mL) was added at 0° C. under N2 atmosphere. The reaction mixture was stirred at room temperature for 3 hours. The reaction mixture was concentrated under reduced pressure to obtain a crude product. The crude product was purified by trituration with diethyl ether (2×20 mL) to afford 3-(6-(4-((2,6-diazaspiro[3.3]heptan-2-yl)methyl)piperidin-1-yl)-1-methyl-1H-indazol-3-yl)piperidine-2,6-dione (0.30 g). LC-MS (ESI): m/z=437.59 [M+H]⁺.

### Preparation of 3-(6-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.3]heptan-2-yl)methyl)piperidin-1-yl)-1-methyl-1H-indazol-3-yl)piperidine-2,6-dione

To a stirred solution of 3-(6-(4-((2,6-diazaspiro[3.3]heptan-2-yl)methyl)piperidin-1-yl)-1-methyl-1H-indazol-3-yl)piperidine-2,6-dione hydrochloride (0.15 g, 0.31 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.07 g, 0.15 mmol) in DMSO (3.0 mL), N,N-diisopropylethylamine (0.17 mL, 0.95 mmol) was added. The reaction was heated at 100° C. for 4 hours. Solvent was removed under vacuum to isolate a crude product. The crude product was purified by prep HPLC to afford 3-(6-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.3]heptan-2-yl)methyl)piperidin-1-yl)-1-methyl-1H-indazol-3-yl)piperidine-2,6-dione (0.045 g) as an off-white solid. 1H NMR (400 MHz, DMSO-d₆): δ 10.83 (s, 1H), 8.80 (s, 1H), 8.18 (s, 1H), 8.14 (s, 1H), 8.04 (s, 1H), 7.85 (dd, J=2.00, 9.20 Hz, 1H), 7.45 (dd, J=9.20, 17.80 Hz, 2H), 6.89 (d, J=9.20 Hz, 1H), 6.82 (s, 1H), 6.14 (s, 1H), 4.51-4.49 (m, 2H), 4.24-4.23 (m, 1H), 4.04 (s, 3H), 3.88 (s, 3H), 3.76 (d, J=12.40 Hz, 2H), 3.39-3.37 (m, 6H), 3.23 (br, 1H), 2.67-2.66 (m, 2H), 2.61-2.59 (m, 2H), 2.54 (s, 3H), δ 2.30-2.28 (m, 1H), 2.17-2.16 (m, 1H), 2.09-2.07 (m, 1H), 1.74 (d, J=11.60 Hz, 1H), 1.27-1.24 (m, 4H), 0.73-0.72 (m, 1H), 0.56-0.55 (m, 2H), 0.37-0.36 (m, 1H). LC-MS (ESI): m/z=866.43 [M−H]⁻.

### Example 46: 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-1-methyl-1H-indazol-3-yl)piperidine-2,6-dione (Compound 132a)

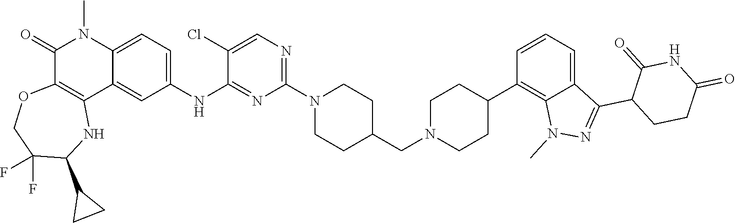 [SMILES: Cn1nc(C2CCC(=O)NC2=O)c2cccc(C3CCN(CC4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-(3-(2,6-bis(benzyloxy)pyridin-3-yl)-1-methyl-1H-indazol-7-yl)-3,6-dihydropyridine-1(2H)-carboxylate

To a stirred solution of 3-(2,6-bis(benzyloxy)pyridin-3-yl)-7-bromo-1-methyl-1H-indazole (2.0 g, 4.0 mmol) in 1,4-dioxane (32.0 mL) and water (8.0 mL), tert-butyl 4-(4,4,5,5-tetramethyl-1,3,2-dioxaborolan-2-yl)-3,6-dihydropyridine-1(2H)-carboxylate (1.48 g, 4.80 mmol) was added. The reaction was purged with nitrogen gas for 20 minutes. Cesium carbonate (3.91 g, 12.01 mmol), XantPhos (0.92 g, 1.60 mmol), and Pd₂(dba)₃ (0.73 g, 0.80 mmol) were added. The reaction was heated at 100° C. for 16 hours. After cooling to room temperature, the reaction was quenched with water and extracted with ethyl acetate. The combined organic layers were washed with brine solution, dried over anhydrous sodium sulfate, filtered, and concentrated under vacuum to obtain a crude product. The crude product was purified by flash column chromatography (22% ethyl acetate in petroleum ether) to afford tert-butyl 4-(3-(2,6-bis(benzyloxy)pyridin-3-yl)-1-methyl-1H-indazol-7-yl)-3,6-dihydropyridine-1(2H)-carboxylate (2.0 g) as a yellow solid. LC-MS (ESI): m/z=603.62 [M+H]⁺.

### Preparation of 3-(2,6-bis(benzyloxy)pyridin-3-yl)-1-methyl-7-(1,2,3,6-tetrahydropyridin-4-yl)-1H-indazole hydrochloride

To a stirred solution of tert-butyl 4-(3-(2,6-bis(benzyloxy)pyridin-3-yl)-1-methyl-1H-indazol-7-yl)-3,6-dihydropyridine-1(2H)-carboxylate (2.0 g, 3.31 mmol) in DCM (20 ml), 4M HCl in 1,4-dioxane (10 ml) was added, and the reaction was stirred at room temperature for 2 hours. The reaction mixture was evaporated under vacuum to obtain a crude product. The crude product was triturated using diethyl ether (3.5 ml) and dried under vacuum to obtained 3-(2,6-bis(benzyloxy)pyridin-3-yl)-1-methyl-7-(1,2,3,6-tetrahydropyridin-4-yl)-1H-indazole hydrochloride (1.3 g) as a yellow gum. LC-MS (ESI): m/z=503.60 [M+H]⁺.

### Preparation of tert-butyl 4-((4-(3-(2,6-bis(benzyloxy)pyridin-3-yl)-1-methyl-1H-indazol-7-yl)-3,6-dihydropyridin-1(2H)-yl)methyl)piperidine-1-carboxylate

To a solution of 3-(2,6-bis(benzyloxy)pyridin-3-yl)-1-methyl-7-(1,2,3,6-tetrahydropyridin-4-yl)-1H-indazole hydrochloride (1.3 g, 2.41 mmol) and tert-butyl 4-formylpiperidine-1-carboxylate (0.40 g, 2.89 mmol) in THF (26 ml), NaCNBH₃ (0.45 g, 7.24 mmol) and Ti(OCH(CH₃)₂)₄(1.3 mL) were added. The reaction was heated at 80° C. for 4 hours. The reaction was quenched with water (5 mL) and extracted with ethyl acetate (15 mL). The combined organic layers were washed with brine solution (5 mL), dried over anhydrous sodium sulfate, filtered, and concentrated under vacuum to obtain a crude product. The crude product was purified by flash column chromatography (18% ethyl acetate in petroleum ether) to afford tert-butyl 4-((4-(3-(2,6-bis(benzyloxy)pyridin-3-yl)-1-methyl-1H-indazol-7-yl)-3,6-dihydropyridin-1(2H)-yl)methyl)piperidine-1-carboxylate (1.0 g) as a yellow solid. LC-MS (ESI): m/z=700.82 [M+H]⁺.

### Preparation of tert-butyl 4-((4-(3-(2,6-dioxopiperidin-3-yl)-1-methyl-1H-indazol-7-yl)piperidin-1-yl)methyl)piperidine-1-carboxylate

To a stirred solution of tert-butyl 4-((4-(3-(2,6-bis(benzyloxy)pyridin-3-yl)-1-methyl-1H-indazol-7-yl)-3,6-dihydropyridin-1(2H)-yl)methyl)piperidine-1-carboxylate (0.3 g, 0.42 mmol) in THF (12 mL), 20% Pd(OH)₂ on carbon (50% wet) (0.3 g) was added. The reaction was put under H₂ atmosphere (15 psi) and stirred at room temperature for 16 hours. The reaction mixture was diluted with ethyl acetate (25 mL) and filtered through celite. The celite was washed with 30% THF in ethyl acetate (15 mL). The filtrate was concentrated under vacuum to obtain a crude product. The crude product was triturated with diethyl ether to afford tert-butyl 4-((4-(3-(2,6-dioxopiperidin-3-yl)-1-methyl-1H-indazol-7-yl)piperidin-1-yl)methyl)piperidine-1-carboxylate (0.21 g) as an off-white solid. LC-MS (ESI): m/z=524.66 [M+H]⁺.

### Preparation of 3-(1-methyl-7-(1-(piperidin-4-ylmethyl) piperidin-4-yl)-1H-indazol-3-yl) piperidine-2,6-dione hydrochloride

A solution of tert-butyl 4-((4-(3-(2,6-dioxopiperidin-3-yl)-1-methyl-1H-indazol-7-yl)piperidin-1-yl)methyl)piperidine-1-carboxylate (0.21 g, 0.40 mmol) in 1,4-dioxane (2.1 mL) was cooled to 0° C. 4M HCl in 1,4-dioxane (1.05 mL) was added, and the reaction mixture was allowed to warm to room temperature over 2 hours with stirring. The reaction mixture was concentrated under vacuum to isolate a crude product. The crude product was triturated using diethyl ether (3 mL) to obtain 3-(1-methyl-7-(1-(piperidin-4-ylmethyl) piperidin-4-yl)-1H-indazol-3-yl) piperidine-2,6-dione hydrochloride (0.16 g). LC-MS (ESI): m/z=424.59 [M+H]⁺.

### Preparation of 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-1-methyl-1H-indazol-3-yl)piperidine-2,6-dione

To a stirred solution of 3-(1-methyl-7-(1-(piperidin-4-ylmethyl) piperidin-4-yl)-1H-indazol-3-yl) piperidine-2,6-dione hydrochloride (0.15 g, 0.32 mmol) in DMSO (6.0 mL), (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.076 g, 0.16 mmol) and DIPEA (0.3 mL, 1.63 mmol) were added. The reaction mixture was heated at 100° C. for 5 hours. Solvent was removed under vacuum to obtained crude product. The crude product was purified by prep HPLC to afford 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-1-methyl-1H-indazol-3-yl)piperidine-2,6-dione (0.028 g) as an off-white solid. 1H NMR (400 MHz, DMSO-d₆): δ 10.88 (s, 1H), 8.78 (s, 1H), 8.19 (d, J=2.0 Hz, 1H), 8.02 (s, 1H), 7.75 (dd, J=1.60, 9.00 Hz, 1H), 7.53 (d, J=8.0 Hz, 1H), 7.45 (d, J=9.2 Hz, 1H), 7.25 (d, J=6.8 Hz, 1H), 7.07 (t, J=7.6 Hz, 1H), 6.16 (br s, 1H), 4.45-4.33 (m, 5H), 4.14 (s, 3H), 3.56 (s, 3H), 3.30-3.21 (m, 2H), 2.96 (br s, 2H), 2.96-2.78 (m, 2H), 2.67-2.65 (m, 2H), 2.18-2.06 (m, 5H), 1.86-1.69 (m, 8H), 1.45-1.41 (m, 1H), 1.03-1.00 (m, 2H), 0.72-0.68 (m, 1H), 0.53-0.50 (m, 2H), 0.42-0.39 (m, 1H). LC-MS (ESI): m/z=855.56 [M+H]⁺.

### Example 47: 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidine-1-carbonyl)-1-methyl-1H-indazol-3-yl)piperidine-2,6-dione (Compound 391a)

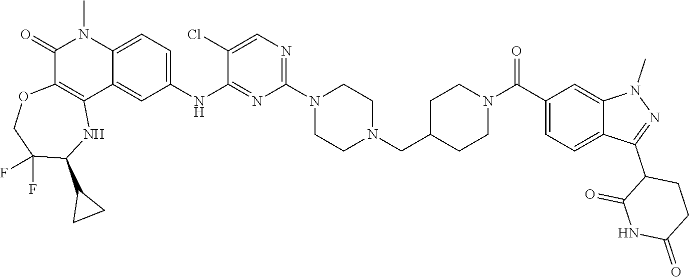 [SMILES: Cn1nc(C2CCC(=O)NC2=O)c2ccc(C(=O)N3CCC(CN4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-((1-(3-(2,6-dioxopiperidin-3-yl)-1-methyl-1H-indazole-6-carbonyl)piperidin-4-yl)methyl)piperazine-1-carboxylate

A mixture of tert-butyl 4-(piperidin-4-ylmethyl)piperazine-1-carboxylate (191 mg, 674 μmol), 3-(2,6-dioxopiperidin-3-yl)-1-methyl-1H-indazole-6-carboxylic acid (176 mg, 613 μmol), and NMI (106 mg, 103 μL, 1.29 mmol) in DMA (3.00 mL) was treated with N,N,N′,N′-tetramethylchloroformamidinium hexafluorophosphate (206 mg, 735 μmol). The reaction mixture was allowed to stir for 3 hours, then filtered and purified by reverse phase chromatography (10-60% MeCN/water [w/0.1% formic acid]) to afford tert-butyl 4-((1-(3-(2,6-dioxopiperidin-3-yl)-1-methyl-1H-indazole-6-carbonyl)piperidin-4-yl)methyl)piperazine-1-carboxylate (282 mg, 0.49 mmol). LC-MS (ESI): m/z=553.5 [M+H]⁺.

### Preparation of 3-(1-methyl-6-(4-(piperazin-1-ylmethyl)piperidine-1-carbonyl)-1H-indazol-3-yl)piperidine-2,6-dione

A mixture of tert-butyl 4-((1-(3-(2,6-dioxopiperidin-3-yl)-1-methyl-1H-indazole-6-carbonyl)piperidin-4-yl)methyl)piperazine-1-carboxylate (280 mg, 507 μmol) and DCM (2.00 mL) was treated with a mixture of DCM (2.00 mL) and TFA (2.00 mL, 26.0 mmol) and stirred at room temperature for 16 hours. The volatiles were removed under vacuum, and the residue was suspended in DCM and concentrated again under vacuum (2×) to afford 3-(1-methyl-6-(4-(piperazin-1-ylmethyl)piperidine-1-carbonyl)-1H-indazol-3-yl)piperidine-2,6-dione (287 mg, 507 μmol) as a TFA salt. LC-MS (ESI): m/z=453.4 [M+H]⁺.

### Preparation of 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidine-1-carbonyl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (50.0 mg, 107 μmol), 3-(1-methyl-6-(4-(piperazin-1-ylmethyl)piperidine-1-carbonyl)-1H-indazol-3-yl)piperidine-2,6-dione (72.5 mg, 160 μmol), and DIEA (93.0 μL, 534 μmol) in DMSO (1.00 mL) was warmed to 80° C. for 17 hours. The reaction mixture was cooled to room temperature, filtered, and purified by prep HPLC (10-59-100% MeCN/water w/0.1% formic acid) to afford 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidine-1-carbonyl)-1-methyl-1H-indazol-3-yl)piperidine-2,6-dione (38.0 mg, 43 μmol) as an off-white solid. 1H NMR (400 MHz, DMSO-d₆): δ 10.90 (s, 1H), 8.84 (s, 1H), 8.21 (d, J=2.2 Hz, 1H), 8.04 (s, 1H), 7.76 (d, J=8.3 Hz, 1H), 7.69 (dd, J=9.1, 2.2 Hz, 1H), 7.64 (s, 1H), 7.43 (d, J=9.1 Hz, 1H), 7.08 (d, J=8.4 Hz, 1H), 6.21 (s, 1H), 4.59-4.30 (m, 4H), 4.02 (s, 3H), 3.56 (s, 4H), 3.26-3.15 (m, 2H), 3.01 (s, 1H), 2.83-2.60 (m, 3H), 2.54 (s, 2H), 2.44-2.26 (m, 4H), 2.23-2.07 (m, 4H), 1.82 (br s, 2H), 1.62 (br s, 1H), 1.37-1.27 (m, 1H), 1.27-1.20 (m, 1H), 1.18-1.00 (m, 2H), 0.77-0.63 (m, 1H), 0.57-0.46 (m, 2H), 0.34 (q, J=6.4 Hz, 1H). LC-MS (ESI): m/z=884.6 [M+H]⁺.

### Example 48: 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)-1-methyl-1H-indazol-3-yl)piperidine-2,6-dione (Compound 392a)

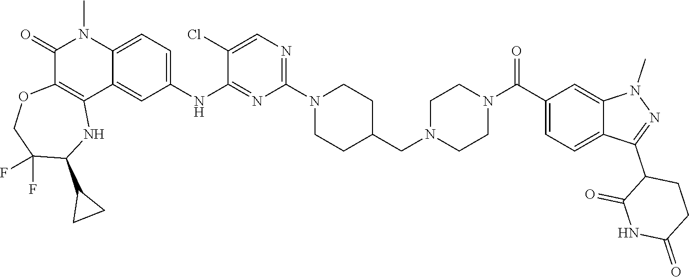 [SMILES: Cn1nc(C2CCC(=O)NC2=O)c2ccc(C(=O)N3CCN(CC4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-((4-(3-(2,6-dioxopiperidin-3-yl)-1-methyl-1H-indazole-6-carbonyl)piperazin-1-yl)methyl)piperidine-1-carboxylate

A mixture of tert-butyl 4-(piperazin-1-ylmethyl)piperidine-1-carboxylate (191 mg, 674 μmol), 3-(2,6-dioxopiperidin-3-yl)-1-methyl-1H-indazole-6-carboxylic acid (176 mg, 613 μmol), and N-methylimidazole (106 mg, 103 μL, 1.29 mmol) in DMA (3.00 mL) was treated with N,N,N′,N′-tetramethylchloroformamidinium hexafluorophosphate (206 mg, 735 μmol). The reaction mixture was allowed to stir for 3 hours. The reaction mixture was diluted with DMSO and filtered. The crude solution was purified by reverse phase chromatography (15-70% MeCN/water [w/0.1% formic acid]) to afford tert-butyl 4-((4-(3-(2,6-dioxopiperidin-3-yl)-1-methyl-1H-indazole-6-carbonyl)piperazin-1-yl)methyl)piperidine-1-carboxylate (232 mg, 420 μmol). LC-MS (ESI): m/z=553.5 [M+H]⁺.

### Preparation of 3-(1-methyl-6-(4-(piperidin-4-ylmethyl)piperazine-1-carbonyl)-1H-indazol-3-yl)piperidine-2,6-dione

A mixture of tert-butyl 4-((4-(3-(2,6-dioxopiperidin-3-yl)-1-methyl-1H-indazole-6-carbonyl)piperazin-1-yl)methyl)piperidine-1-carboxylate (230 mg, 416 μmol) and DCM (2.00 mL) was treated with a mixture of DCM (2.00 mL) and TFA (2.00 mL, 26.0 mmol) and stirred at room temperature for 16 hours. The volatiles were removed under vacuum, and the residue was suspended in DCM and concentrated under vacuum (2×) to afford 3-(1-methyl-6-(4-(piperidin-4-ylmethyl)piperazine-1-carbonyl)-1H-indazol-3-yl)piperidine-2,6-dione (236 mg, 416 μmol) as a TFA salt. LC-MS (ESI): m/z=453.3 [M+H]⁺.

### Preparation of 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (50.0 mg, 107 μmol), 3-(1-methyl-6-(4-(piperidin-4-ylmethyl)piperazine-1-carbonyl)-1H-indazol-3-yl)piperidine-2,6-dione (72.5 mg, 160 μmol), and DIEA (93.0 μL, 534 μmol) in DMSO (1.00 mL) was warmed to 80° C. for 17 hours. The reaction mixture was cooled to room temperature, filtered, and purified by prep HPLC (10-73-100% MeCN/water w/0.1% formic acid) to afford 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)-1-methyl-1H-indazol-3-yl)piperidine-2,6-dione (37.4 mg, 42 μmol) as an off-white solid. 1H NMR (400 MHz, DMSO-d₆): δ 10.90 (s, 1H), 8.76 (s, 1H), 8.17 (d, J=2.2 Hz, 1H), 8.02 (s, 1H), 7.83-7.69 (m, 2H), 7.67 (s, 1H), 7.44 (d, J=9.1 Hz, 1H), 7.09 (d, J=8.3 Hz, 1H), 6.16 (s, 1H), 4.54-4.28 (m, 5H), 4.02 (s, 3H), 3.72-3.59 (m, 2H), 3.56 (s, 3H), 3.26-3.14 (m, 1H), 2.75 (t, J=13.0 Hz, 2H), 2.69-2.58 (m, 1H), 2.54 (s, 2H), 2.45-2.25 (m, 4H), 2.24-2.08 (m, 2H), 1.79-1.64 (m, 3H), 1.38-1.15 (m, 1H), 1.00 (d, J=12.6 Hz, 2H), 0.79-0.64 (m, 1H), 0.51 (t, J=6.2 Hz, 2H), 0.34 (q, J=6.6 Hz, 1H). LC-MS (ESI): m/z=884.6 [M+H]⁺.

### Example 49: 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidine-1-carbonyl)-1-methyl-1H-indazol-3-yl)piperidine-2,6-dione (Compound 393a)

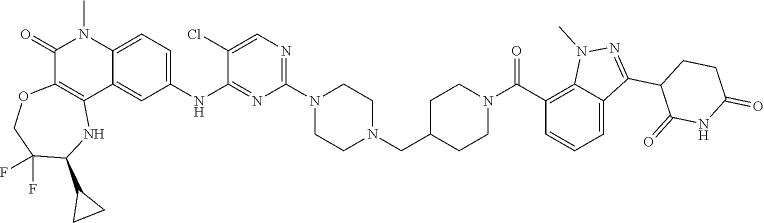 [SMILES: Cn1nc(C2CCC(=O)NC2=O)c2cccc(C(=O)N3CCC(CN4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-((1-(3-(2,6-dioxopiperidin-3-yl)-1-methyl-1H-indazole-7-carbonyl)piperidin-4-yl)methyl)piperazine-1-carboxylate

A mixture of tert-butyl 4-(piperidin-4-ylmethyl)piperazine-1-carboxylate (191 mg, 674 μmol), 3-(2,6-dioxopiperidin-3-yl)-1-methyl-1H-indazole-7-carboxylic acid (176 mg, 613 μmol), and N-methylimidazole (103 μL, 1.29 mmol) in DMA (3.00 mL) was treated with N,N,N′,N′-tetramethylchloroformamidinium hexafluorophosphate (206 mg, 735 μmol). The reaction mixture was allowed to stir for 3 hours, diluted with DMSO, filtered, and purified by reverse phase chromatography (20-70% MeCN/water [w/0.1% formic acid]) to afford tert-butyl 4-((1-(3-(2,6-dioxopiperidin-3-yl)-1-methyl-1H-indazole-7-carbonyl)piperidin-4-yl)methyl)piperazine-1-carboxylate (251 mg, 454 μmol) as a beige solid. LC-MS (ESI): m/z=553.5 [M+H]⁺.

### Preparation of 3-(1-methyl-7-(4-(piperazin-1-ylmethyl)piperidine-1-carbonyl)-1H-indazol-3-yl)piperidine-2,6-dione

A mixture of tert-butyl 4-((1-(3-(2,6-dioxopiperidin-3-yl)-1-methyl-1H-indazole-7-carbonyl)piperidin-4-yl)methyl)piperazine-1-carboxylate (249 mg, 451 μmol) and DCM (2.00 mL) was treated with a mixture of DCM (2.00 mL) and TFA (2.00 mL, 26.0 mmol) and stirred at room temperature for 16 hours. The volatiles were removed under vacuum, and the residue was suspended in DCM and concentrated again under vacuum (2×) to afford 3-(1-methyl-7-(4-(piperazin-1-ylmethyl)piperidine-1-carbonyl)-1H-indazol-3-yl)piperidine-2,6-dione (255 mg, 451 μmol) as a TFA salt. LC-MS (ESI): m/z=453.4 [M+H]⁺.

### Preparation of 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidine-1-carbonyl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (50.0 mg, 107 μmol), 3-(1-methyl-7-(4-(piperazin-1-ylmethyl)piperidine-1-carbonyl)-1H-indazol-3-yl)piperidine-2,6-dione (72.5 mg, 160 μmol), and DIEA (69.0 mg, 93.0 μL, 5.00 equiv., 534 μmol) in DMSO (1.00 mL) was warmed to 80° C. for 17 hours. The reaction mixture was cooled to room temperature, filtered, and purified by prep HPLC (10-28-36-100% MeCN/water w/0.1% formic acid) to afford 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidine-1-carbonyl)-1-methyl-1H-indazol-3-yl)piperidine-2,6-dione (42.2 mg, 47 μmol) as a pale beige solid. 1H NMR (400 MHz, DMSO-d₆): δ 10.91 (s, 1H), 8.84 (s, 1H), 8.21 (t, J=2.4 Hz, 1H), 8.04 (s, 1H), 7.79 (t, J=7.7 Hz, 1H), 7.68 (dd, J=9.0, 2.1 Hz, 1H), 7.43 (d, J=9.1 Hz, 1H), 7.27 (dd, J=13.6, 7.0 Hz, 1H), 7.16 (t, J=7.6 Hz, 1H), 6.21 (s, 1H), 4.61 (t, J=14.5 Hz, 1H), 4.52-4.27 (m, 3H), 3.89 (d, J=24.6 Hz, 3H), 3.56 (s, 6H), 3.27-3.09 (m, 2H), 3.00 (t, J=12.6 Hz, 1H), 2.86 (d, J=11.0 Hz, 1H), 2.66 (d, J=8.3 Hz, 2H), 2.54 (s, 2H), 2.33 (d, J=6.2 Hz, 4H), 2.17 (s, 4H), 1.85 (s, 2H), 1.61 (s, 1H), 1.31 (t, J=17.3 Hz, 1H), 1.20-1.04 (m, 1H), 0.69 (d, J=8.0 Hz, 1H), 0.50 (t, J=6.2 Hz, 2H), 0.34 (d, J=5.3 Hz, 1H). LC-MS (ESI): m/z=884.6 [M+H]⁺.

### Example 50: 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)-1-methyl-1H-indazol-3-yl)piperidine-2,6-dione (Compound 394a)

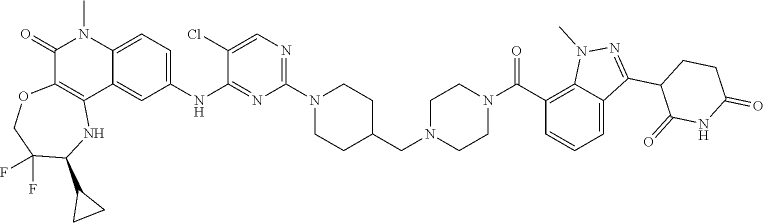 [SMILES: Cn1nc(C2CCC(=O)NC2=O)c2cccc(C(=O)N3CCN(CC4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-((4-(3-(2,6-dioxopiperidin-3-yl)-1-methyl-1H-indazole-7-carbonyl)piperazin-1-yl)methyl)piperidine-1-carboxylate

A mixture of tert-butyl 4-(piperazin-1-ylmethyl)piperidine-1-carboxylate (191 mg, 674 μmol), 3-(2,6-dioxopiperidin-3-yl)-1-methyl-1H-indazole-7-carboxylic acid (176 mg, 613 μmol), and N-methylimidazole (103 μL, 1.29 mmol) in DMA (3.00 mL) was treated with N,N,N′,N′-tetramethylchloroformamidinium hexafluorophosphate (206 mg, 735 μmol). The reaction mixture was allowed to stir for 3 hours, diluted with DMSO, filtered, and purified by reverse phase chromatography (20-55% MeCN/water [w/0.1% formic acid]) to afford tert-butyl 4-((4-(3-(2,6-dioxopiperidin-3-yl)-1-methyl-1H-indazole-7-carbonyl)piperazin-1-yl)methyl)piperidine-1-carboxylate (332 mg, 601 μmol). LC-MS (ESI): m/z=553.5 [M+H]⁺.

### Preparation of 3-(1-methyl-7-(4-(piperidin-4-ylmethyl)piperazine-1-carbonyl)-1H-indazol-3-yl)piperidine-2,6-dione

A mixture of tert-butyl 4-((4-(3-(2,6-dioxopiperidin-3-yl)-1-methyl-1H-indazole-7-carbonyl)piperazin-1-yl)methyl)piperidine-1-carboxylate (330 mg, 597 μmol) and DCM (2.00 mL) was treated with a mixture of DCM (2.00 mL) and TFA (2.00 mL, 26.0 mmol) and stirred at room temperature for 16 hours. The volatiles were removed under vacuum, and the residue was suspended in DCM and concentrated again under vacuum (2×) to afford 3-(1-methyl-7-(4-(piperidin-4-ylmethyl)piperazine-1-carbonyl)-1H-indazol-3-yl)piperidine-2,6-dione (338 mg, 597 μmol) as a TFA salt. LC-MS (ESI): m/z=453.4 [M+H]⁺.

### Preparation of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (50.0 mg, 107 μmol), 3-(1-methyl-7-(4-(piperidin-4-ylmethyl)piperazine-1-carbonyl)-1H-indazol-3-yl)piperidine-2,6-dione (72.5 mg, 160 μmol), and DIEA (93.0 μL, 534 μmol) in DMSO (1.00 mL) was warmed to 80° C. for 17 hours. The reaction mixture was cooled to room temperature, filtered, and purified by prep HPLC (10-28-33-100% MeCN/water w/0.1% formic acid) to afford 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazine-1-carbonyl)-1-methyl-1H-indazol-3-yl)piperidine-2,6-dione (46.5 mg, 52 μmol). 1H NMR (400 MHz, DMSO-d₆): δ 10.91 (s, 1H), 8.76 (s, 1H), 8.16 (d, J=2.3 Hz, 1H), 8.01 (s, 1H), 7.81 (d, J=7.6 Hz, 1H), 7.72 (dd, J=9.1, 2.1 Hz, 1H), 7.43 (d, J=9.2 Hz, 1H), 7.28 (d, J=6.8 Hz, 1H), 7.21-7.11 (m, 1H), 6.16 (s, 1H), 4.60-4.19 (m, 4H), 3.89 (s, 3H), 3.83-3.74 (m, 1H), 3.74-3.64 (m, 1H), 3.56 (s, 3H), 3.29-3.14 (m, 2H), 2.76 (t, J=12.6 Hz, 2H), 2.70-2.62 (m, 2H), 2.54 (s, 2H), 2.45-2.28 (m, 4H), 2.26-2.18 (m, 2H), 2.15 (d, J=6.7 Hz, 2H), 1.83-1.62 (m, 3H), 1.39-1.26 (m, 1H), 1.09-0.92 (m, 2H), 0.70 (q, J=5.7 Hz, 1H), 0.55-0.45 (m, 2H), 0.34 (q, J=6.2 Hz, 1H). LC-MS (ESI): m/z=884.6 [M+H]⁺.

### Example 51: 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(trifluoromethyl)piperazin-1-yl)methyl)piperidin-1-yl)-1-methyl-1H-indazol-3-yl)piperidine-2,6-dione (Compound 426a)

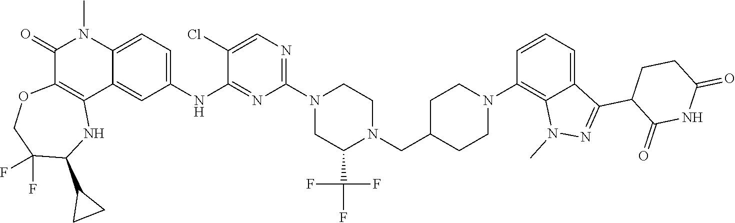 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@H]4C(F)(F)F)CC3)c21]

### Preparation of tert-butyl (S)-4-((1-((benzyloxy)carbonyl)piperidin-4-yl)methyl)-3-(trifluoromethyl)piperazine-1-carboxylate

A one dram vial containing decaborane(14) (13.9 mg, 114 μmol) was charged with tert-butyl (S)-3-(trifluoromethyl)piperazine-1-carboxylate (106 mg, 418 μmol) and benzyl 4-formylpiperidine-1-carboxylate (94.0 mg, 380 μmol). A stir bar was added, followed by MeOH (2.00 mL). The reaction mixture was stirred at room temperature for 3 hours, then quenched by the addition of 0.5M HCl (1.0 mL). The aqueous layer was extracted with DCM (3×), the organic layers were concentrated under reduced pressure, and the residue purified by column chromatography (0-100% (3:1 ethyl acetate/EtOH)/heptane) to afford tert-butyl (S)-4-((1-((benzyloxy)carbonyl)piperidin-4-yl)methyl)-3-(trifluoromethyl)piperazine-1-carboxylate (160 mg, 330 μmol). LC-MS (ESI): m/z=486.3 [M+H]⁺.

### Preparation of tert-butyl (S)-4-(piperidin-4-ylmethyl)-3-(trifluoromethyl)piperazine-1-carboxylate

To a 20 mL vial containing tert-butyl (S)-4-((1-((benzyloxy)carbonyl)piperidin-4-yl)methyl)-3-(trifluoromethyl)piperazine-1-carboxylate (157.3 mg, 324.0 μmol) was added isopropanol (3.00 mL) and ethyl acetate (1.50 mL). The solution was sparged with N2 for 5 minutes then treated with Pd(OH)₂ on carbon (20% wt, 100.0 mg, 142.4 μmol). The mixture was then sparged with H₂ for 5 minutes, then stirred under a balloon of H₂ for 20 hours. The reaction mixture was sparged with N2, filtered through celite, and rinsed with ethyl acetate, methanol, and DCM (20 mL each).

The filtrate was concentrated under vacuum to afford a crude residue, which was dissolved in DMSO and purified by reverse phase chromatography (10-100% MeCN/water [w/0.1% formic acid]) to afford tert-butyl (S)-4-(piperidin-4-ylmethyl)-3-(trifluoromethyl)piperazine-1-carboxylate (88.0 mg, 250 μmol). LC-MS (ESI): m/z=352.3 [M+H]⁺.

### Preparation of tert-butyl (S)-4-((1-(3-(2,6-bis(benzyloxy)pyridin-3-yl)-1-methyl-1H-indazol-7-yl)piperidin-4-yl)methyl)-3-(trifluoromethyl)piperazine-1-carboxylate

A mixture of tert-butyl (S)-4-(piperidin-4-ylmethyl)-3-(trifluoromethyl)piperazine-1-carboxylate (85.9 mg, 244 μmol), 3-(2,6-bis(benzyloxy)pyridin-3-yl)-7-bromo-1-methyl-1H-indazole (147 mg, 293 μmol), and RuPhos-Pd-G3 (15.2 mg, 18.2 μmol) in THF (1.50 mL) was sparged with N₂ for 5 minutes. A solution of potassium tert-butoxide (1.00 Min THF, 709 μL, 709 μmol) was added and the reaction mixture was warmed to 80° C. for 3 hours. The reaction mixture was cooled to room temperature, treated with formic acid (55.3 μL, 1.47 mmol), and purified by column chromatography (0-100% (3:1 ethyl acetate/EtOH)/heptane) to afford tert-butyl (S)-4-((1-(3-(2,6-bis(benzyloxy)pyridin-3-yl)-1-methyl-1H-indazol-7-yl)piperidin-4-yl)methyl)-3-(trifluoromethyl)piperazine-1-carboxylate (116 mg, 0.150 mmol).

### Preparation of tert-butyl (3S)-4-((1-(3-(2,6-dioxopiperidin-3-yl)-1-methyl-1H-indazol-7-yl)piperidin-4-yl)methyl)-3-(trifluoromethyl)piperazine-1-carboxylate

A solution of tert-butyl (S)-4-((1-(3-(2,6-bis(benzyloxy)pyridin-3-yl)-1-methyl-1H-indazol-7-yl)piperidin-4-yl)methyl)-3-(trifluoromethyl)piperazine-1-carboxylate (116 mg, 150 μmol) in ethyl acetate (1.00 mL), DMF (1.00 mL), and IPA (1.00 mL) was sparged with N₂ for 5 minutes, treated with Pd/C (10% wt, 46.0 mg, 43.2 μmol), and then sparged with H₂ for 5 minutes.

The reaction mixture was then stirred under an atmosphere of H₂ at 50° C. for 20 hours, cooled to room temperature, sparged w/N2 for five minutes, charged with Pd/C (10% wt, 96.0 mg, 90.2 μmol), sparged with H₂ for five minutes, and warmed under an atmosphere of H₂ for 20 hours. The reaction mixture was cooled to room temperature, filtered through celite (using DCM, then MeOH, then ethyl acetate), concentrated under reduced pressure, dissolved in DMSO, filtered, then purified by prep HPLC (50-100% MeCN/water [w/0.1% formic acid]) to afford tert-butyl (3S)-4-((1-(3-(2,6-dioxopiperidin-3-yl)-1-methyl-1H-indazol-7-yl)piperidin-4-yl)methyl)-3-(trifluoromethyl)piperazine-1-carboxylate (73.0 mg, 123 μmol). LC-MS (ESI): m/z=593.8 [M+H]⁺.

### Preparation of 3-(1-methyl-7-(4-(((S)-2-(trifluoromethyl)piperazin-1-yl)methyl)piperidin-1-yl)-1H-indazol-3-yl)piperidine-2,6-dione

A solution of tert-butyl (3S)-4-((1-(3-(2,6-dioxopiperidin-3-yl)-1-methyl-1H-indazol-7-yl)piperidin-4-yl)methyl)-3-(trifluoromethyl)piperazine-1-carboxylate (68.7 mg, 116 μmol) in DCM (1.00 mL) was treated with a 1:1 solution of TFA (1.00 mL, 13.0 mmol) and DCM (1.00 mL) and the reaction mixture stirred for 3 hours. The volatiles were removed under reduced pressure, the residue suspended in DCM, and the volatiles removed under reduced pressure (2×) to afford 3-(1-methyl-7-(4-(((S)-2-(trifluoromethyl)piperazin-1-yl)methyl)piperidin-1-yl)-1H-indazol-3-yl)piperidine-2,6-dione bis-trifluoroacetate (91.8 mg, 0.11 mmol). LC-MS (ESI): m/z=493.6 [M+H]⁺.

### Preparation of 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(trifluoromethyl)piperazin-1-yl)methyl)piperidin-1-yl)-1-methyl-1H-indazol-3-yl)piperidine-2,6-dione

A solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (69.4 mg, 148 mol), 3-(1-methyl-7-(4-(((S)-2-(trifluoromethyl)piperazin-1-yl)methyl)piperidin-1-yl)-1H-indazol-3-yl)piperidine-2,6-dione bis-trifluoroacetate (39.5 mg, 65.1 mol), and DIEA (45.4 μL, 261 mol) in DMSO (0.700 mL) was warmed to 80° C. for 18 hours. The reaction mixture was cooled to room temperature, filtered, and purified by prep HPLC (39-100% MeCN/water [w/0.1% formic acid]) to afford 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(trifluoromethyl)piperazin-1-yl)methyl)piperidin-1-yl)-1-methyl-1H-indazol-3-yl)piperidine-2,6-dione (31.4 mg, 34 mol). 1H NMR (400 MHz, DMSO-d₆): δ 10.87 (s, 1H), 8.88 (s, 1H), 8.17 (s, 1H), 8.06 (s, 1H), 7.72 (dd, J=9.0, 2.2 Hz, 1H), 7.41 (d, J=9.0 Hz, 1H), 7.36 (dd, J=6.4, 2.6 Hz, 1H), 7.05-6.96 (m, 2H), 6.20 (s, 1H), 4.53-4.35 (m, 2H), 4.33 (dd, J=9.5, 5.1 Hz, 1H), 4.23 (s, 3H), 3.99-3.84 (m, 1H), 3.56 (s, 3H), 3.49 (d, J=12.2 Hz, 2H), 3.28-3.18 (m, 6H), 2.96-2.86 (m, 1H), 2.79-2.56 (m, 6H), 2.38-2.25 (m, 1H), 2.23-2.11 (m, 1H), 1.86 (t, J=13.8 Hz, 2H), 1.75-1.60 (m, 1H), 1.47-1.25 (m, 3H), 0.76-0.65 (m, 1H), 0.58-0.46 (m, 2H), 0.43-0.27 (m, 1H). LC-MS (ESI): m/z=924.5 [M+H]⁺.

### Example 52: 4-(3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)azetidin-1-yl)-2-(2,6-dioxopiperidin-3-yl)isoindoline-1,3-dione (Compound 433a)

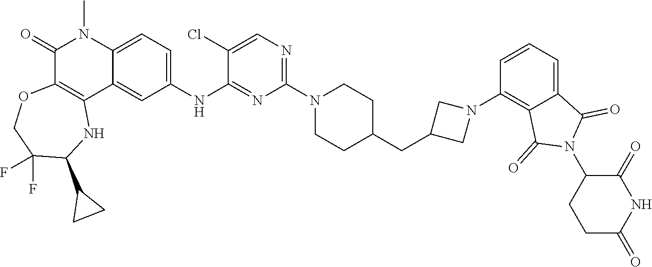 [SMILES: Cn1c(=O)c2c(c3cc(Nc4nc(N5CCC(CC6CN(c7cccc8c7C(=O)N(C7CCC(=O)NC7=O)C8=O)C6)CC5)ncc4Cl)ccc31)N[C@@H](C1CC1)C(F)(F)CO2]

### Preparation of tert-butyl 4-((1-(2-(2,6-dioxopiperidin-3-yl)-1,3-dioxoisoindolin-4-yl)azetidin-3-yl)methyl)piperidine-1-carboxylate

A mixture of tert-butyl 4-(azetidin-3-ylmethyl)piperidine-1-carboxylate hydrochloride (80.0 mg, 275 μmol), 2-(2,6-dioxopiperidin-3-yl)-4-fluoroisoindoline-1,3-dione (86.0 mg, 311 μmol), and DIEA (144 μL, 825 μmol) in DMSO (1.80 mL) was warmed to 100° C. for 24 hours, then cooled to room temperature. The reaction mixture was purified directly by column chromatography (0-100% (3:1 ethyl acetate/EtOH)/heptane) to afford tert-butyl 4-((1-(2-(2,6-dioxopiperidin-3-yl)-1,3-dioxoisoindolin-4-yl)azetidin-3-yl)methyl)piperidine-1-carboxylate (139 mg, 0.27 mmol).

LC-MS (ESI): m/z=511.6 [M+H]⁺.

### Preparation of 2-(2,6-dioxopiperidin-3-yl)-4-(3-(piperidin-4-ylmethyl)azetidin-1-yl)isoindoline-1,3-dione

A solution of tert-butyl 4-((1-(2-(2,6-dioxopiperidin-3-yl)-1,3-dioxoisoindolin-4-yl)azetidin-3-yl)methyl)piperidine-1-carboxylate (146 mg, 286 μmol) in DCM (1.00 mL) was treated with a 1:1 solution of TFA (1.00 mL, 13.0 mmol) and DCM (1.00 mL) and the reaction mixture stirred for 18 hours. The volatiles were removed under reduced pressure, the residue dissolved in DCM and concentrated under reduced pressure (3×) to afford 2-(2,6-dioxopiperidin-3-yl)-4-(3-(piperidin-4-ylmethyl)azetidin-1-yl)isoindoline-1,3-dione (179 mg, 280 μmol) as a yellow solid (TFA salt).

LC-MS (ESI): m/z=411.4 [M+H]⁺.

### Preparation of 4-(3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)azetidin-1-yl)-2-(2,6-dioxopiperidin-3-yl)isoindoline-1,3-dione

A solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (95.3 mg, 203 mol), 2-(2,6-dioxopiperidin-3-yl)-4-(3-(piperidin-4-ylmethyl)azetidin-1-yl)isoindoline-1,3-dione, TFA (64.4 mg, 101 mol), and DIEA (87.8 μL, 504 mol) in DMSO (0.700 mL) was warmed to 80° C. for 19 hours, then allowed to cool to room temperature. The reaction mixture was treated with formic acid, filtered, and purified by prep HPLC (10-100% MeCN/water [w/0.1% acetic acid]) to afford 4-(3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)azetidin-1-yl)-2-(2,6-dioxopiperidin-3-yl)isoindoline-1,3-dione (24.7 mg, 29 mol) as a yellow solid. ¹H NMR (400 MHz, DMSO-d₆): δ 10.99 (s, 1H), 8.71 (s, 1H), 8.11 (d, J=2.3 Hz, 1H), 7.95 (s, 1H), 7.65 (dd, J=9.1, 2.2 Hz, 1H), 7.48 (dd, J=8.5, 7.0 Hz, 1H), 7.37 (d, J=9.1 Hz, 1H), 7.02 (d, J=6.9 Hz, 1H), 6.68 (d, J=8.5 Hz, 1H), 6.13 (s, 1H), 4.96 (dd, J=12.7, 5.5 Hz, 1H), 4.48-4.30 (m, 3H), 4.30-4.19 (m, 3H), 3.69 (t, J=7.5 Hz, 2H), 3.49 (s, 3H), 3.20-3.09 (m, 2H), 2.87-2.73 (m, 1H), 2.72-2.59 (m, 3H), 2.55-2.50 (m, 1H), 2.50-2.47 (m, 2H), 1.96-1.87 (m, 1H), 1.54 (d, J=12.6 Hz, 2H), 1.50-1.37 (m, 1H), 1.31-1.19 (m, 1H), 1.06-0.91 (m, 2H), 0.73-0.58 (m, 1H), 0.51-0.39 (m, 2H), 0.33-0.22 (m, 1H). LC-MS (ESI): m/z=842.5 [M+H]⁺.

### Example 53: 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(trifluoromethyl)piperazin-1-yl)methyl)piperidin-1-yl)-1-methyl-1H-indazol-3-yl)piperidine-2,6-dione (Compound 426b)

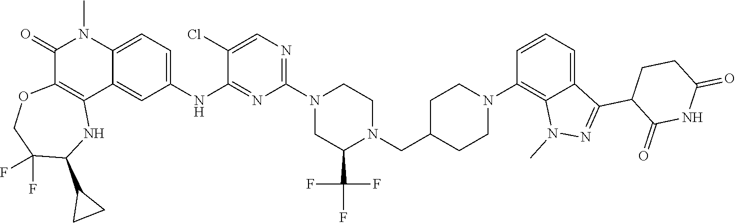 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@@H]4C(F)(F)F)CC3)c21]

### Preparation of 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(trifluoromethyl)piperazin-1-yl)methyl)piperidin-1-yl)-1-methyl-1H-indazol-3-yl)piperidine-2,6-dione

This compound was prepared in a manner similar to that described for Compound 426a to afford 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(trifluoromethyl)piperazin-1-yl)methyl)piperidin-1-yl)-1-methyl-1H-indazol-3-yl)piperidine-2,6-dione (33.4 mg, 36 mol) as a white solid. 1H NMR (400 MHz, DMSO-d₆): δ 10.87 (s, 1H), 8.85 (s, 1H), 8.17 (s, 1H), 8.07 (s, 1H), 7.73 (dd, J=9.1, 2.2 Hz, 1H), 7.41 (d, J=9.1 Hz, 1H), 7.36 (dd, J=6.2, 2.8 Hz, 1H), 7.09-6.93 (m, 2H), 6.21 (s, 1H), 4.51-4.36 (m, 2H), 4.33 (dd, J=9.6, 5.1 Hz, 1H), 4.23 (s, 3H), 3.92 (s, 1H), 3.56 (s, 3H), 3.54-3.43 (m, 3H), 3.28-3.16 (m, 4H), 2.99-2.86 (m, 1H), 2.80-2.59 (m, 8H), 2.37-2.26 (m, 1H), 2.16 (dd, J=13.2, 5.8 Hz, 1H), 1.92-1.80 (m, 2H), 1.74-1.58 (m, 1H), 1.44-1.27 (m, 2H), 0.76-0.66 (m, 1H), 0.58-0.46 (m, 2H), 0.35 (q, J=6.0 Hz, 1H). LC-MS (ESI): m/z=924.5 [M+H]⁺.

### Example 54: 3-(6-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)-1-methyl-1H-indazol-3-yl)piperidine-2,6-dione (Compound 456a)

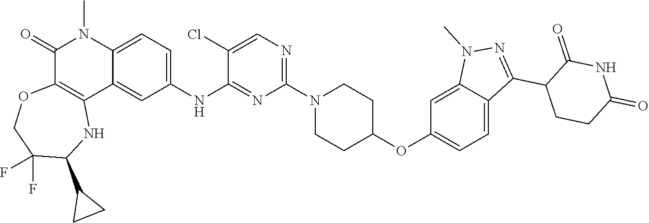 [SMILES: Cn1nc(C2CCC(=O)NC2=O)c2ccc(OC3CCN(c4ncc(Cl)c(Nc5ccc6c(c5)c5c(c(=O)n6C)OCC(F)(F)[C@H](C6CC6)N5)n4)CC3)cc21]

### Preparation of 3-(6-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (46.0 mg, 98.2 mol) and 3-(1-methyl-6-(piperidin-4-yloxy)-1H-indazol-3-yl)piperidine-2,6-dione hydrochloride (44.7 mg, 118 mol) in DMSO (0.700 mL) was treated with DIEA (68.4 [L, 393 mol) and warmed to 80° C. for 19 hours. The reaction mixture was cooled to room temperature, filtered, and purified by prep HPLC (27-57-59-100% MeCN/water [w/0.1% formic acid]) to afford 3-(6-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)-1-methyl-1H-indazol-3-yl)piperidine-2,6-dione (62.5 mg, 77 mol) as an off-white solid. 1H NMR (400 MHz, DMSO-d₆): δ 10.85 (s, 1H), 8.83 (s, 1H), 8.18 (d, J=2.2 Hz, 1H), 8.06 (s, 1H), 7.73 (dd, J=9.1, 2.1 Hz, 1H), 7.55 (d, J=8.9 Hz, 1H), 7.43 (d, J=9.1 Hz, 1H), 7.11 (d, J=2.0 Hz, 1H), 6.74 (dd, J=8.8, 2.0 Hz, 1H), 6.18 (s, 1H), 4.81-4.66 (m, 1H), 4.48-4.32 (m, 1H), 4.29 (dd, J=9.6, 5.1 Hz, 1H), 4.15-3.98 (m, 2H), 3.92 (s, 3H), 3.54 (s, 3H), 3.50-3.40 (m, 2H), 3.27-3.14 (m, 2H), 2.71-2.57 (m, 2H), 2.37-2.23 (m, 1H), 2.20-2.12 (m, 1H), 2.05-1.92 (m, 2H), 1.70-1.55 (m, 2H), 1.40-1.25 (m, 1H), 0.74-0.61 (m, 1H), 0.55-0.43 (m, 2H), 0.40-0.29 (m, 1H). LC-MS (ESI): m/z=774.3 [M+H]⁺.

### Example 55: 3-(7-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)-1-methyl-1H-indazol-3-yl)piperidine-2,6-dione (Compound 457a)

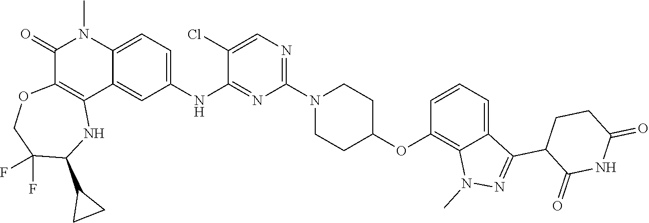 [SMILES: Cn1nc(C2CCC(=O)NC2=O)c2cccc(OC3CCN(c4ncc(Cl)c(Nc5ccc6c(c5)c5c(c(=O)n6C)OCC(F)(F)[C@H](C6CC6)N5)n4)CC3)c21]

### Preparation of 3-(7-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)-1-methyl-1H-indazol-3-yl)piperidine-2,6-dione

This compound was prepared in a manner similar to that described for Compound 456a to afford 3-(7-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)-1-methyl-1H-indazol-3-yl)piperidine-2,6-dione (16.8 mg, 21 mol) as a white solid. 1H NMR (400 MHz, DMSO-d₆): δ 10.87 (s, 1H), 8.84 (s, 1H), 8.18 (d, J=2.3 Hz, 1H), 8.06 (s, 1H), 7.73 (dd, J=9.0, 2.1 Hz, 1H), 7.43 (d, J=9.1 Hz, 1H), 7.21 (d, J=8.0 Hz, 1H), 6.97 (t, J=7.8 Hz, 1H), 6.91 (d, J=7.6 Hz, 1H), 6.20 (s, 1H), 4.84 (s, 1H), 4.54-4.35 (m, 1H), 4.35-4.28 (m, 1H), 4.17 (s, 3H), 4.01-3.84 (m, 2H), 3.69-3.59 (m, 2H), 3.54 (s, 3H), 3.27-3.11 (m, 2H), 2.71-2.58 (m, 2H), 2.40-2.25 (m, 1H), 2.20-2.11 (m, 1H), 2.05-1.94 (m, 2H), 1.84-1.68 (m, 2H), 1.38-1.24 (m, 1H), 0.75-0.59 (m, 1H), 0.58-0.41 (m, 2H), 0.40-0.26 (m, 1H). LC-MS (ESI): m/z=774.3 [M+H]⁺.

### Example 56: 3-(7-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbonyl)piperazin-1-yl)-1-methyl-1H-indazol-3-yl)piperidine-2,6-dione (Compound 425a)

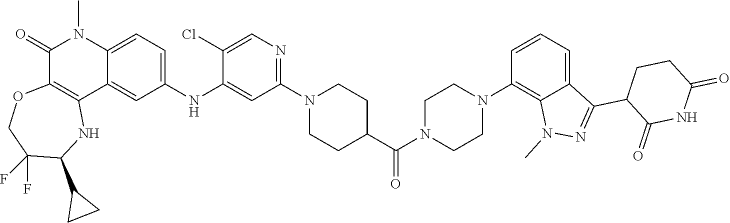 [SMILES: Cn1nc(C2CCC(=O)NC2=O)c2cccc(N3CCN(C(=O)C4CCN(c5cc(Nc6ccc7c(c6)c6c(c(=O)n7C)OCC(F)(F)[C@H](C7CC7)N6)c(Cl)cn5)CC4)CC3)c21]

### Preparation of 3-(7-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbonyl)piperazin-1-yl)-1-methyl-1H-indazol-3-yl)piperidine-2,6-dione

In a small vial equipped with stir bar, a solution of (S)-10-((5-chloro-2-fluoropyridin-4-yl)amino)-2-cyclopropyl-3,3-difluoro-7-methyl-1,2,3,4-tetrahydro-[1,4]oxazepino[2,3-c]quinolin-6(7H)-one (90.2 mg, 200 μmol), 3-(1-methyl-7-(4-(piperidine-4-carbonyl)piperazin-1-yl)-1H-indazol-3-yl)piperidine-2,6-dione, formic acid (96.9 mg, 0.200 mmol), and DIPEA (174 μL, 1.00 mmol) in DMSO (1.5 mL) was stirred at 110° C. in a heated reaction block overnight. The reaction mixture was filtered through a 0.45 μm hydrophobic PTFE syringe filter. The filtrate was purified by prep-HPLC using a gradient of 10-100% MeCN (w/0.1% formic acid) in water (w/0.1% formic acid). Fractions containing the desired product were evaporated and lyophilized to give 3-(7-(4-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbonyl)piperazin-1-yl)-1-methyl-1H-indazol-3-yl)piperidine-2,6-dione (23.0 mg) as a white solid. 1H NMR (400 MHz, DMSO-d₆) δ 10.88 (s, 1H), 8.10 (s, 1H), 7.97 (s, 1H), 7.57-7.35 (m, 3H), 7.03 (d, J=6.6 Hz, 2H), 6.42 (s, 1H), 6.07 (s, 1H), 4.44 (dd, J=15.1, 5.7 Hz, 1H), 4.35 (dt, J=9.9, 5.0 Hz, 1H), 4.26 (s, 3H), 4.03 (m, 2H), 3.58 (s, 3H), 3.21 (dt, J=13.6, 6.9 Hz, 1H), 2.96-2.73 (m, 3H), 2.73-2.59 (m, 2H), 2.41-2.26 (m, 1H), 2.16 (dd, J=12.9, 6.1 Hz, 1H), 1.66 (d, J=12.5 Hz, 2H), 1.53 (d, J=14.3 Hz, 2H), 1.38-1.19 (m, 1H), 0.78-0.63 (m, 1H), 0.51 (d, J=6.4 Hz, 2H), 0.30 (q, J=5.6 Hz, 1H). LC-MS (ESI): m/z=870.1 [M+H]⁺.

### Example 57: 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-fluoropiperidin-4-yl)methyl)piperidin-4-yl)-1-methyl-1H-indazol-3-yl)piperidine-2,6-dione (Compound 461a)

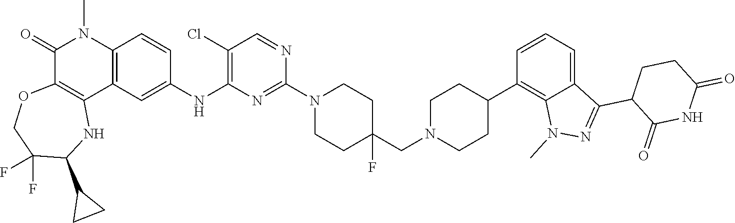 [SMILES: Cn1nc(C2CCC(=O)NC2=O)c2cccc(C3CCN(CC4(F)CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-(3-(2,6-bis(benzyloxy)pyridin-3-yl)-1-methyl-1H-indazol-7-yl)-3,6-dihydropyridine-1(2H)-carboxylate

In a 250 mL round-bottomed flask equipped with stir bar, a mixture of tert-butyl 4-(4,4,5,5-tetramethyl-1,3,2-dioxaborolan-2-yl)-3,6-dihydropyridine-1(2H)-carboxylate (1.55 g, 5.00 mmol), 3-(2,6-bis(benzyloxy)pyridin-3-yl)-7-bromo-1-methyl-1H-indazole (2.50 g, 5.00 mmol), and cesium carbonate (4.89 g, 15.0 mmol) in 1,4-dioxane (20 mL) and water (5.0 mL) was treated with Xantphos (289 mg, 500 mol) and Pd₂(dba)₃ (229 mg, 250 mol). The reaction mixture was stirred under nitrogen atmosphere at 100° C. in a heated reaction block overnight. The reaction mixture was allowed to cool to room temperature and was diluted with water, then extracted 3X with ethyl acetate. The combined organics were dried over sodium sulfate, filtered, and concentrated under reduced pressure. The residue was purified by flash chromatography using a gradient of 20-100% ethyl acetate in heptanes. Fractions were evaporated to give tert-butyl 4-(3-(2,6-bis(benzyloxy)pyridin-3-yl)-1-methyl-1H-indazol-7-yl)-3,6-dihydropyridine-1(2H)-carboxylate (2.70 g) as a yellow foam. LC-MS (ESI): m/z=603.3 [M+H]⁺.

### Preparation of tert-butyl 4-(3-(2,6-dioxopiperidin-3-yl)-1-methyl-1H-indazol-7-yl)piperidine-1-carboxylate

A solution of tert-butyl 4-(3-(2,6-bis(benzyloxy)pyridin-3-yl)-1-methyl-1H-indazol-7-yl)-3,6-dihydropyridine-1(2H)-carboxylate (2.70 g, 4.48 mmol) in THF (30 mL) was treated with Pd(OH)₂ on carbon (786 mg, 20 wt %, 1.12 mmol). Hydrogen gas (balloon) was bubbled through the solution for several minutes and the reaction mixture was stirred at 50° C. under hydrogen atmosphere in a heated reaction block for 24 hours. The reaction mixture was removed from heat and was filtered through celite. The filtrate was taken up in DMSO and filtered through a 0.45 μm hydrophilic PTFE syringe filter. The filtrate was purified by reverse phase column chromatography using a gradient of 10-100% MeCN (w/0.1% formic acid) in water (w/0.1% formic acid). Fractions were evaporated to give tert-butyl 4-(3-(2,6-dioxopiperidin-3-yl)-1-methyl-1H-indazol-7-yl)piperidine-1-carboxylate (0.946 g). LC-MS (ESI): m/z=427.3 [M+H]⁺.

### Preparation of 3-(1-methyl-7-(piperidin-4-yl)-1H-indazol-3-yl)piperidine-2,6-dione

A suspension of tert-butyl 4-(3-(2,6-dioxopiperidin-3-yl)-1-methyl-1H-indazol-7-yl)piperidine-1-carboxylate (0.946 g, 2.22 mmol) in 1,4-dioxane (10 mL) was treated with HCl (4M in dioxane, 5.54 mL, 22.2 mmol), and the reaction mixture was stirred at room temperature overnight. The reaction mixture was concentrated under reduced pressure to give crude 3-(1-methyl-7-(piperidin-4-yl)-1H-indazol-3-yl)piperidine-2,6-dione, 2 HCl (899 mg) as a white solid, which was used without purification. LC-MS (ESI): m/z=327.1 [M+H]⁺.

### Preparation of tert-butyl 4-((4-(3-(2,6-dioxopiperidin-3-yl)-1-methyl-1H-indazol-7-yl)piperidin-1-yl)methyl)-4-fluoropiperidine-1-carboxylate

A mixture of 3-(1-methyl-7-(piperidin-4-yl)-1H-indazol-3-yl)piperidine-2,6-dione, 2 HCl (639 mg, 98 wt %, 1.57 mmol), DIPEA (546 μL, 3.14 mmol), and tert-butyl 4-fluoro-4-formylpiperidine-1-carboxylate (363 mg, 1.57 mmol) in THF (20 mL) was treated with acetic acid (269 μL, 4.70 mmol) and sodium triacetoxyborohydride (499 mg, 2.35 mmol). The reaction mixture was stirred at room temperature overnight. The reaction mixture was diluted with water and extracted 3X with ethyl acetate. The combined organics were dried over sodium sulfate, filtered, and concentrated under reduced pressure. The residue was taken up in DMSO and filtered through a 0.45 μm hydrophilic PTFE filter. The filtrate was purified by prep HPLC using a gradient of 10-100% MeCN (w/0.1% acetic acid) in water (w/0.1% acetic acid) over two injections. Fractions containing product were evaporated to give tert-butyl 4-((4-(3-(2,6-dioxopiperidin-3-yl)-1-methyl-1H-indazol-7-yl)piperidin-1-yl)methyl)-4-fluoropiperidine-1-carboxylate (0.299 g) as a pale yellow foam. LC-MS (ESI): m/z=542.3 [M+H]⁺.

### Preparation of 3-(7-(1-((4-fluoropiperidin-4-yl)methyl)piperidin-4-yl)-1-methyl-1H-indazol-3-yl)piperidine-2,6-dione

A solution of tert-butyl 4-((4-(3-(2,6-dioxopiperidin-3-yl)-1-methyl-1H-indazol-7-yl)piperidin-1-yl)methyl)-4-fluoropiperidine-1-carboxylate (0.299 g, 552 mol) in DCM (5 mL) was treated with TFA (851 μL, 11.0 mmol) and stirred at room temperature overnight. The reaction mixture was concentrated under reduced pressure. The residue was taken up in methanol, then concentrated under reduced pressure again to give 3-(7-(1-((4-fluoropiperidin-4-yl)methyl)piperidin-4-yl)-1-methyl-1H-indazol-3-yl)piperidine-2,6-dione, TFA salt (306 mg) as a pale orange semi-solid, which was used without further purification. LC-MS (ESI): m/z=442.3 [M+H]⁺.

### Preparation of 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-fluoropiperidin-4-yl)methyl)piperidin-4-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (129 mg, 275 mol), 3-(7-(1-((4-fluoropiperidin-4-yl)methyl)piperidin-4-yl)-1-methyl-1H-indazol-3-yl)piperidine-2,6-dione, TFA salt (153 mg, 275 mol), and DIPEA (240 μL, 1.38 mmol) in DMSO (1.8 mL) was stirred at 80° C. in a heated reaction block overnight. The reaction mixture was filtered through a 0.45 μm hydrophilic PTFE filter. The filtrate was purified by prep HPLC using a gradient of 20-100% MeCN (w/0.1% formic acid) in water (w/0.1% formic acid). Fractions containing product were evaporated and lyophilized to give 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-fluoropiperidin-4-yl)methyl)piperidin-4-yl)-1-methyl-1H-indazol-3-yl)piperidine-2,6-dione (121.6 mg) as an off-white solid. 1H NMR (400 MHz, DMSO-d₆) δ 10.87 (s, 1H), 8.84 (s, 1H), 8.21 (d, J=2.3 Hz, 1H), 8.06 (s, 1H), 7.72 (dd, J=9.1, 2.1 Hz, 1H), 7.54 (d, J=8.0 Hz, 1H), 7.45 (d, J=9.1 Hz, 1H), 7.24 (d, J=7.1 Hz, 1H), 7.06 (t, J=7.6 Hz, 1H), 6.19 (d, J=4.3 Hz, 1H), 4.50-4.38 (m, 1H), 4.34 (dd, J=9.7, 5.0 Hz, 1H), 4.20 (s, 3H), 3.57 (s, 3H), 3.24-3.13 (m, 2H), 3.05 (s, 2H), 2.68-2.59 (m, 1H), 2.34 (dp, J=14.1, 4.9 Hz, 2H), 2.22-2.09 (m, 1H), 1.96-1.53 (m, 7H), 1.35 (dd, J=10.9, 6.0 Hz, 1H), 0.70 (q, J=5.6 Hz, 1H), 0.58-0.47 (m, 2H), 0.35 (q, J=5.6 Hz, 1H). LC-MS (ESI): m/z=873.5 [M+H]⁺.

### Example 58: 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-fluoropiperidin-4-yl)methyl)piperidin-4-yl)-1-methyl-1H-indazol-3-yl)piperidine-2,6-dione (Compound 472a)

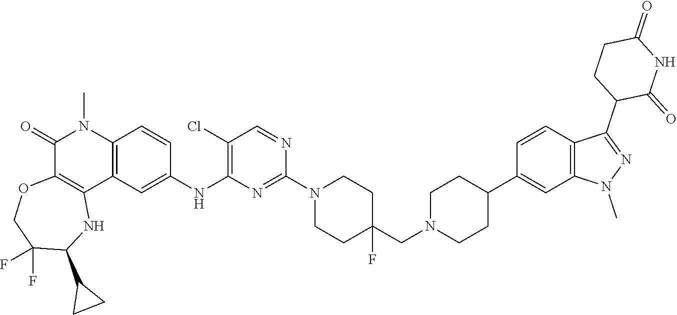 [SMILES: Cn1nc(C2CCC(=O)NC2=O)c2ccc(C3CCN(CC4(F)CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-((4-(3-(2,6-dioxopiperidin-3-yl)-1-methyl-1H-indazol-6-yl)piperidin-1-yl)methyl)-4-fluoropiperidine-1-carboxylate

THF (20 mL) and DMF (3.0 mL) were added to a flask containing tert-butyl 4-fluoro-4-formylpiperidine-1-carboxylate (927 mg, 4.01 mmol), 3-(1-methyl-6-(piperidin-4-yl)-1H-indazol-3-yl)piperidine-2,6-dione, 2 HCl (800 mg, 2.00 mmol, prepared using published methods, e.g., International Application No. WO2022235945), and potassium acetate (590 mg, 6.01 mmol) at room temperature and stirred for 30 minutes. Then, sodium triacetoxyborohydride (637 mg, 3.01 mmol) was added in one portion, and the reaction mixture was stirred at room temperature overnight. The reaction was quenched with saturated aqueous NH₄Cl and extracted 3X with ethyl acetate. The combined organics were washed with water, then brine, dried over sodium sulfate, filtered, and concentrated under reduced pressure. The residue was purified by flash column chromatography using a gradient of 40-100% ethyl acetate in heptanes for 13.5 minutes, then eluted with 3:1 ethyl acetate:EtOH. Fractions were evaporated to give tert-butyl 4-((4-(3-(2,6-dioxopiperidin-3-yl)-1-methyl-1H-indazol-6-yl)piperidin-1-yl)methyl)-4-fluoropiperidine-1-carboxylate (0.573 g) as a colorless oil that crashed out to a white solid. LC-MS (ESI): m/z=542.4 [M+H]⁺.

### Preparation of 3-(6-(1-((4-fluoropiperidin-4-yl)methyl)piperidin-4-yl)-1-methyl-1H-indazol-3-yl)piperidine-2,6-dione

A solution of tert-butyl 4-((4-(3-(2,6-dioxopiperidin-3-yl)-1-methyl-1H-indazol-6-yl)piperidin-1-yl)methyl)-4-fluoropiperidine-1-carboxylate (0.573 g, 1.06 mmol) in DCM (11 mL) was treated with trifluoroacetic acid (1.63 mL, 21.2 mmol). The reaction mixture was stirred at room temperature for 3.5 hours. The reaction mixture was concentrated under reduced pressure. The residue was taken up in methanol and concentrated under reduced pressure again to give 3-(6-(1-((4-fluoropiperidin-4-yl)methyl)piperidin-4-yl)-1-methyl-1H-indazol-3-yl)piperidine-2,6-dione, TFA (820 mg) as an orange oil, which was used without further purification. LC-MS (ESI): m/z=442.3 [M+H]⁺.

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-fluoropiperidin-4-yl)methyl)piperidin-4-yl)-1-methyl-1H-indazol-3-yl)piperidine-2,6-dione

In a vial equipped with stir bar, a mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (124 mg, 265 mol), 3-(6-(1-((4-fluoropiperidin-4-yl)methyl)piperidin-4-yl)-1-methyl-1H-indazol-3-yl)piperidine-2,6-dione, TFA (210 mg, 70 wt %, 265 mol), and DIPEA (230 μL, 1.32 mmol) in DMSO (2.65 mL) was stirred at 80° C. in a heated reaction block overnight. The reaction mixture was filtered through a 0.45 μm hydrophilic PTFE syringe filter. The filtrate was purified by prep HPLC using a gradient of 10-100% MeCN (w/0.1% acetic acid) in water (w/0.1% acetic acid). Fractions containing product were evaporated and lyophilized to give 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-fluoropiperidin-4-yl)methyl)piperidin-4-yl)-1-methyl-1H-indazol-3-yl)piperidine-2,6-dione (119.2 mg) as a pink solid. 1H NMR (400 MHz, DMSO-d₆) δ 10.87 (s, 1H), 8.83 (s, 1H), 8.25-8.18 (m, 1H), 8.06 (s, 1H), 7.72 (dd, J=9.0, 2.1 Hz, 1H), 7.60 (d, J=8.3 Hz, 1H), 7.50-7.36 (m, 2H), 7.02 (d, J=8.4 Hz, 1H), 6.32-6.09 (m, 1H), 4.54-4.29 (m, 3H), 4.17 (m, 2H), 3.96 (s, 3H), 3.57 (s, 3H), 3.26-3.11 (m, 3H), 3.00 (d, J=10.5 Hz, 2H), 2.73-2.55 (m, 2H), 2.42-2.30 (m, 1H), 2.27-2.11 (m, 3H), 1.88-1.56 (m, 7H), 1.34 (p, J=6.1 Hz, 1H), 0.71 (q, J=5.9 Hz, 1H), 0.57-0.46 (m, 2H), 0.35 (q, J=6.2 Hz, 1H). LC-MS (ESI): m/z=873.5 [M+H]⁺.

### Example 59: 3-(6-(1-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-4-fluoropiperidin-4-yl)methyl)piperidin-4-yl)-1-methyl-1H-indazol-3-yl)piperidine-2,6-dione (Compound 474a)

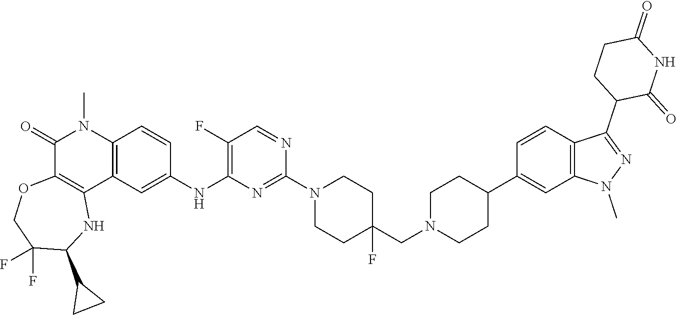 [SMILES: Cn1nc(C2CCC(=O)NC2=O)c2ccc(C3CCN(CC4(F)CCN(c5ncc(F)c(Nc6ccc7c(c6)c6c(c(=O)n7C)OCC(F)(F)[C@H](C7CC7)N6)n5)CC4)CC3)cc21]

### Preparation of 3-(6-(1-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-4-fluoropiperidin-4-yl)methyl)piperidin-4-yl)-1-methyl-1H-indazol-3-yl)piperidine-2,6-dione

A mixture of (S)-10-((2-chloro-5-fluoropyrimidin-4-yl)amino)-2-cyclopropyl-3,3-difluoro-7-methyl-1,2,3,4-tetrahydro-[1,4]oxazepino [2,3-c]quinolin-6(7H)-one (114 mg, 1 equiv., 252 mol), 3-(6-(1-((4-fluoropiperidin-4-yl)methyl)piperidin-4-yl)-1-methyl-1H-indazol-3-yl)piperidine-2,6-dione, TFA (200 mg, 70 wt %, 252 mol), cesium fluoride (38.3 mg, 252 mol), and DIPEA (163 mg, 219 μL, 1.26 mmol) in DMSO (2 mL) was stirred at 110° C. in a heated reaction block overnight. The reaction mixture was removed from heat and was filtered through a 0.45 μm hydrophilic PTFE syringe filter. The filtrate was purified by prep HPLC using a gradient of 10-100% MeCN (w/0.1% acetic acid) in water (w/0.1% acetic acid), then a gradient of 10-100% MeCN (w/0.1% formic acid) in water (w/0.1% formic acid). The product was repurified by prep HPLC using a gradient of 10-100% MeCN (w/0.1% TFA) in water (w/0.1% TFA). Fractions containing product were evaporated and lyophilized to give 3-(6-(1-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-4-fluoropiperidin-4-yl)methyl)piperidin-4-yl)-1-methyl-1H-indazol-3-yl)piperidine-2,6-dione, TFA (41.7 mg, 16%, 93% purity) as a white solid. 1H NMR (400 MHz, DMSO-d₆) δ 10.88 (s, 1H), 9.52 (s, 1H), 9.31 (s, 1H), 8.23 (d, J=2.3 Hz, 1H), 8.09 (d, J=3.7 Hz, 1H), 7.77 (dd, J=9.1, 2.2 Hz, 1H), 7.68 (d, J=8.4 Hz, 1H), 7.52-7.34 (m, 2H), 7.03 (dd, J=8.5, 1.3 Hz, 1H), 6.16 (d, J=4.5 Hz, 1H), 4.54-4.40 (m, 1H), 4.39-4.21 (m, 3H), 3.98 (d, J=2.8 Hz, 3H), 3.64 (d, J=11.7 Hz, 2H), 3.57 (s, 4H), 3.34-3.15 (m, 5H), 2.96 (t, J=12.2 Hz, 1H), 2.76-2.56 (m, 2H), 2.42-2.28 (m, 1H), 2.21-2.08 (m, 2H), 2.08-1.90 (m, 4H), 1.78 (dd, J=35.4, 12.8 Hz, 2H), 1.32 (d, J=4.6 Hz, 1H), 0.72 (q, J=6.1 Hz, 1H), 0.59-0.47 (m, 2H), 0.35 (q, J=5.9 Hz, 1H). LC-MS (ESI): m/z=857.5 [M+H]⁺.

### Example 60. 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)piperidin-1-yl)-1-methyl-1H-indazol-3-yl)piperidine-2,6-dione (Compound 416a)

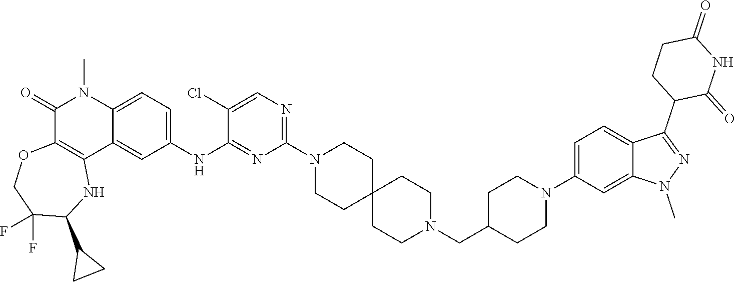 [SMILES: Cn1nc(C2CCC(=O)NC2=O)c2ccc(N3CCC(CN4CCC5(CC4)CCN(c4ncc(Cl)c(Nc6ccc7c(c6)c6c(c(=O)n7C)OCC(F)(F)[C@H](C7CC7)N6)n4)CC5)CC3)cc21]

### Preparation of tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate

To a stirred solution of 3-(2,6-bis(benzyloxy)pyridin-3-yl)-6-bromo-1-methyl-1H-indazole (1.0 g, 2.0 mmol) and tert-butyl 9-(piperidin-4-ylmethyl)-3,9-diazaspiro[5.5]undecane-3-carboxylate (1.40 g, 4.0 mmol, prepared using published methods, e.g., International Application No.

WO2022228547) in 1,4-dioxane (20 mL), cesium carbonate (1.95 g, 6.0 mmol) was added. The reaction was degassed with argon for 10 minutes. RuPhos (0.09 g, 0.20 mmol) and RuPhos-PdG3 (0.08 g, 0.10 mmol) were added, and the reaction was degassed for 5 minutes. The reaction mixture was stirred at 80° C. for 3 hours. The reaction mixture was cooled to room temperature and filtered through celite. The filtrate was concentrated to isolate a crude product. The crude product was purified by flash column chromatography (30% ethyl acetate in petroleum ether) to afford tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate (0.7 g) as an off-white solid. LC-MS (ESI): m/z=771.83 [M+H]⁺.

### Preparation of tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate

To a stirred solution of tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate (0.7 g, 0.90 mmol) in THF (28 mL), 20% Pd(OH)₂ on carbon (0.7 g) was added at room temperature. The reaction was put under hydrogen pressure (60 psi) and stirred for 8 hours at room temperature. The reaction mixture was filtered through celite. The celite was washed with ethyl acetate and concentrated under reduced pressure to isolate a crude product. The crude product was washed with n-pentane and dried to afford tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate (0.60 g) as a brown solid.

LC-MS (ESI): m/z=593.67 [M+H]⁺.

### Preparation of 3-(6-(4-((3,9-diazaspiro[5.5]undecan-3-yl)methyl)piperidin-1-yl)-1-methyl-1H-indazol-3-yl)piperidine-2,6-dione

A stirred solution of tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate (0.6 g, 1.01 mmol) in DCM (12 mL) was cooled to 0° C. TFA (6 mL) was added and the reaction mixture was allowed to warm to room temperature over 3 hours with stirring. The reaction mixture was concentrated under reduced pressure to isolate a crude product. The crude product was triturated with diethyl ether (20 mL) to afford 3-(6-(4-((3,9-diazaspiro[5.5]undecan-3-yl)methyl)piperidin-1-yl)-1-methyl-1H-indazol-3-yl)piperidine-2,6-dione (0.44 g) as a brown solid. LC-MS (ESI): m/z=493.37 [M+H]⁺.

### Preparation of 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)piperidin-1-yl)-1-methyl-1H-indazol-3-yl)piperidine-2,6-dione

To a stirred solution of 3-(6-(4-((3,9-diazaspiro[5.5]undecan-3-yl)methyl)piperidin-1-yl)-1-methyl-1H-indazol-3-yl)piperidine-2,6-dione (0.22 g, 0.44 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.17 mmol) in DMSO (3 mL), N,N-diisopropylethylamine (0.8 mL, 3.57 mmol) was added. The reaction mixture was stirred at 100° C. for 4 hours. Solvent was removed under vacuum to isolate crude product. The crude product was purified by prep HPLC to afford 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)piperidin-1-yl)-1-methyl-1H-indazol-3-yl)piperidine-2,6-dione (15.6 mg) as an off-white solid. 1H NMR (400 MHz, DMSO-d6): δ 10.84 (s, 1H), 8.77 (s, 1H), 8.42 (s, 1H), 8.02 (s, 1H), 7.72 (d, J=9.2 Hz, 1H), 7.47-7.43 (m, 2H), 6.88 (d, J=9.2 Hz, 1H), 6.80 (s, 1H), 6.18 (s, 1H), 4.60-4.30 (m, 2H), 4.25-4.15 (m, 1H), 3.80 (s, 3H), 3.75-3.70 (m 2H), 3.55 (s, 7H), 2.80-2.68 (m, 5H), 2.59-2.55 (m, 5H), 2.16-2.14 (m, 3H), 1.80-1.60 (m, 2H), 1.52-1.20 (m, 12H), 0.85-0.72 (m, 1H), 0.63-0.51 (m, 2H), 0.41-0.38 (m, 1H). LC-MS (ESI): m/z=924.55 [M+H]⁺.

### Example 61.3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperazin-1-yl)-1-methyl-1H-indazol-3-yl)piperidine-2,6-dione (Compound 453a)

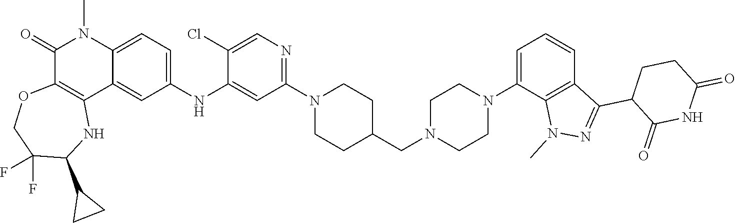 [SMILES: Cn1nc(C2CCC(=O)NC2=O)c2cccc(N3CCN(CC4CCN(c5cc(Nc6ccc7c(c6)c6c(c(=O)n7C)OCC(F)(F)[C@H](C7CC7)N6)c(Cl)cn5)CC4)CC3)c21]

### Preparation of tert-butyl 4-(3-(2,6-bis(benzyloxy)pyridin-3-yl)-1-methyl-1H-indazol-7-yl)piperazine-1-carboxylate

To a stirred solution of 3-(2,6-bis(benzyloxy)pyridin-3-yl)-7-bromo-1-methyl-1H-indazole (0.60 g, 1.20 mmol) and tert-butyl piperazine-1-carboxylate (0.55 g, 3.00 mmol) in 1,4-dioxane (18.0 mL), cesium carbonate (1.17 g, 3.60 mmol) was added. The reaction was degassed with argon gas for 15 minutes. Pd-PEPPSI-iHeptCl (0.06 g, 0.06 mmol) was added, and the reaction was stirred at 100° C. for 16 hours. The reaction mixture was cooled to room temperature, diluted with ethyl acetate (50 mL), and filtered through celite. The celite was washed with 10% MeOH:DCM (200 mL), and the filtrate was concentrated to obtain a crude product. The crude was purified by flash chromatography (10-30% ethyl acetate in petroleum ether) to afford tert-butyl 4-(3-(2,6-bis(benzyloxy)pyridin-3-yl)-1-methyl-1H-indazol-7-yl)piperazine-1-carboxylate (0.38 g) as a yellow solid. LC-MS (ESI): m/z=606.65 [M+H]⁺.

### Preparation of tert-butyl 4-(3-(2,6-dioxopiperidin-3-yl)-1-methyl-1H-indazol-7-yl)piperazine-1-carboxylate

To a solution of tert-butyl 4-(3-(2,6-bis(benzyloxy)pyridin-3-yl)-1-methyl-1H-indazol-7-yl)piperazine-1-carboxylate (0.38 g, 0.62 mmol) in THF (23.0 mL), 20% Pd(OH)₂ on carbon (0.38 g, 100% w/w) was added. The reaction was put under hydrogen atmosphere (85 psi) and stirred at room temperature for 16 hours. The reaction mixture was diluted with THF (30 mL) and filtered through celite. The celite was washed with THF:DCM (1:1, 200 mL), and the filtrate was concentrated to afford tert-butyl 4-(3-(2,6-dioxopiperidin-3-yl)-1-methyl-1H-indazol-7-yl)piperazine-1-carboxylate (0.31 g) as a black solid. LC-MS (ESI): m/z=428.51 [M+H]⁺.

### Preparation of 3-(1-methyl-7-(piperazin-1-yl)-1H-indazol-3-yl)piperidine-2,6-dione

A stirred solution of tert-butyl 4-(3-(2,6-dioxopiperidin-3-yl)-1-methyl-1H-indazol-7-yl)piperazine-1-carboxylate (0.31 g, 0.72 mmol) in DCM (6.2 mL) was cooled to 0° C. TFA (3.1 mL) was added, and the reaction mixture was allowed to warm to room temperature over 3 hours with stirring. The reaction mixture was concentrated under reduced pressure and co-distilled with DCM (2×10 mL) to obtain a crude product. The crude product was triturated with diethyl ether (2×10 mL) to afford 3-(1-methyl-7-(piperazin-1-yl)-1H-indazol-3-yl)piperidine-2,6-dione as a brown semi-solid (0.38 g as a TFA salt). LC-MS (ESI): m/z=328.48 [M+H]⁺.

### Preparation of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperazin-1-yl)-1-methyl-1H-indazol-3-yl)piperidine-2,6-dione

To a stirred solution of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde (0.20 g, 0.36 mmol) and 3-(1-methyl-7-(piperazin-1-yl)-1H-indazol-3-yl)piperidine-2,6-dione (0.14 g, 0.44 mmol) in DCM (12.0 mL), triethylamine (1.0 mL) was added. The reaction was heated at 40° C. for 1 hour. The reaction was cooled to 0° C. and sodium triacetoxyborohydride (0.23 g, 1.10 mmol) was added. The reaction mixture was allowed to warm to room temperature over 4 hours. The reaction mixture was concentrated under reduced pressure to obtain a crude product. The crude product was purified by prep HPLC purification to afford 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperazin-1-yl)-1-methyl-1H-indazol-3-yl)piperidine-2,6-dione (0.038 g) as an off-white solid. 1H NMR (400 MHz, DMSO-d6): δ 10.89 (s, 1H), 8.38 (br s, 1H), 8.00 (s, 1H), 7.51-7.45 (m, 2H), 7.39-7.37 (m, 2H), 7.02-7.01 (m, 2H), 6.41 (s, 1H), 6.09 (s, 1H), 4.42-4.31 (m, 3H), 4.23 (s, 3H), 3.98 (t, J=13.60 Hz, 2H), 3.58 (s, 3H), 3.11-2.86 (m, 6H), 2.71-2.60 (m, 6H), 2.33-2.17 (m, 5H), 1.72 (d, J=10.80 Hz, 3H), 1.36-1.27 (m, 1H), 1.07-1.04 (m, 2H), 0.71-0.68 (m, 1H), 0.51-0.49 (m, 2H), 0.35-0.28 (m, 1H). LC-MS (ESI): m/z=855.48 [M-+[ ]⁺.

### Example 62. 3-(7-((1r,5s)-8-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-8-azabicyclo[3.2.1]octan-3-yl)-1-methyl-1H-indazol-3-yl)piperidine-2,6-dione (Compound 444a)

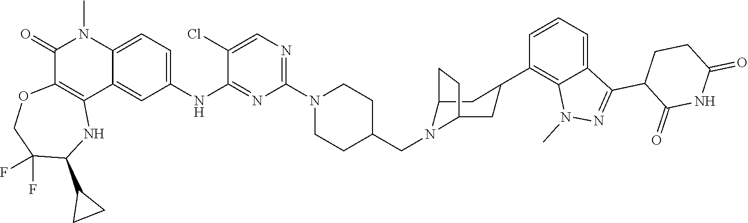 [SMILES: Cn1nc(C2CCC(=O)NC2=O)c2cccc(C3CC4CCC(C3)N4CC3CCN(c4ncc(Cl)c(Nc5ccc6c(c5)c5c(c(=O)n6C)OCC(F)(F)[C@H](C6CC6)N5)n4)CC3)c21]

### Preparation of tert-butyl (1s,5r)-3-(3-(2,6-bis(benzyloxy)pyridin-3-yl)-1-methyl-1H-indazol-7-yl)-8-azabicyclo[3.2.1]oct-2-ene-8-carboxylate

To a stirred solution of 3-(2,6-bis(benzyloxy)pyridin-3-yl)-7-bromo-1-methyl-1H-indazole (1.20 g, 2.40 mmol) and tert-butyl 3-oxa-7,9-diazabicyclo[3.3.1]nonane-7-carboxylate (0.96 g, 2.88 mmol) in 1,4-dioxane (9.6 mL) and water (2.4 mL) was added sodium carbonate (0.76 g, 7.20 mmol). The reaction was purged with N2 for 10 minutes, Pd(dppf)Cl₂ (0.18 g, 0.24 mmol) was added, and the resultant reaction mixture was stirred at 100° C. for 16 hours. The reaction mixture was diluted with ethyl acetate (30 mL) and filtered through celite. The celite was washed with 10% MeOH in DCM (100 mL). The filtrate was concentrated to obtain crude product. The crude product was purified by flash column (10-20% ethyl acetate in petroleum ether) to afford tert-butyl (1s,5r)-3-(3-(2,6-bis(benzyloxy)pyridin-3-yl)-1-methyl-1H-indazol-7-yl)-8-azabicyclo[3.2.1]oct-2-ene-8-carboxylate (1.02 g) as a yellow solid. LC-MS (ESI): m/z=629.58 [M+H]⁺.

### Preparation of 5-(7-((1s,5r)-8-azabicyclo[3.2.1]oct-2-en-3-yl)-1-methyl-1H-indazol-3-yl)-6-(benzyloxy)pyridin-2-ol

To a stirred solution of tert-butyl (1s,5r)-3-(3-(2,6-bis(benzyloxy)pyridin-3-yl)-1-methyl-1H-indazol-7-yl)-8-azabicyclo[3.2.1]oct-2-ene-8-carboxylate (1.0 g, 1.60 mmol) in DCM (20.0 mL) was added 4M HCl in dioxane (10.0 mL) at 0° C. The reaction was allowed to warm to room temperature over 16 hours with stirring. The reaction mixture was concentrated under vacuum to obtain a crude product. The crude product was triturated with diethyl ether to afford 5-(7-((1s,5r)-8-azabicyclo[3.2.1]oct-2-en-3-yl)-1-methyl-1H-indazol-3-yl)-6-(benzyloxy)pyridin-2-ol (0.85 g) as an off-white solid. LC-MS (ESI): m/z=439.29 [M+H]⁺.

### Preparation of tert-butyl 4-(((1s,5r)-3-(3-(2-(benzyloxy)-6-hydroxypyridin-3-yl)-1-methyl-1H-indazol-7-yl)-8-azabicyclo[3.2.1]oct-2-en-8-yl)methyl)piperidine-1-carboxylate

To a stirred solution of 5-(7-((1s,5r)-8-azabicyclo[3.2.1]oct-2-en-3-yl)-1-methyl-1H-indazol-3-yl)-6-(benzyloxy)pyridin-2-ol (0.70 g, 1.60 mmol) and tert-butyl 4-formylpiperidine-1-carboxylate (1.02 g, 4.80 mmol) in DCM (14.0 mL) was added triethylamine (1.10 mL, 7.98 mmol). The reaction was stirred at 40° C. for 2 hours. After cooling to 0° C., sodium triacetoxyborohydride (0.67 g, 3.19 mmol) was added, and the reaction mixture was allowed to warm to room temperature over 4 hours with stirring. The reaction mixture was quenched with NH₄Cl solution (30 mL) and extracted with DCM (2×50 mL). The combined organic layers were washed with brine solution, dried over anhydrous sodium sulfate, filtered, and concentrated to obtain a crude product. The crude was triturated with diethyl ether to afford tert-butyl 4-(((1s,5r)-3-(3-(2-(benzyloxy)-6-hydroxypyridin-3-yl)-1-methyl-1H-indazol-7-yl)-8-azabicyclo[3.2.1]oct-2-en-8-yl)methyl)piperidine-1-carboxylate (1.52 g) as an off-white solid. LC-MS (ESI): m/z=636.48 [M+H]⁺.

### Preparation of tert-butyl 4-(((1r,5s)-3-(3-(2,6-dioxopiperidin-3-yl)-1-methyl-1H-indazol-7-yl)-8-azabicyclo[3.2.1]octan-8-yl)methyl)piperidine-1-carboxylate

To a solution of tert-butyl 4-(((1s,5r)-3-(3-(2-(benzyloxy)-6-hydroxypyridin-3-yl)-1-methyl-1H-indazol-7-yl)-8-azabicyclo[3.2.1]oct-2-en-8-yl)methyl)piperidine-1-carboxylate (1.10 g, 1.73 mmol) in THF (66.0 mL) and acetic acid (0.1 mL, 1.73 mmol) was added 20% Pd(OH)₂ on carbon (1.65 g, 150% w/w). The reaction was put under a hydrogen atmosphere (80 psi) at room temperature and stirred for 16 hours. The reaction mixture was diluted with THF (50 mL) and filtered through celite. The celite was washed with THF:DCM (1:1, 200 mL). The filtrate was concentrated and dried to obtain tert-butyl 4-(((1r,5s)-3-(3-(2,6-dioxopiperidin-3-yl)-1-methyl-1H-indazol-7-yl)-8-azabicyclo[3.2.1]octan-8-yl)methyl)piperidine-1-carboxylate (0.65 g) as a pale yellow semi-solid. LC-MS (ESI): m/z=550.46 [M+H]⁺.

### Preparation of 3-(1-methyl-7-((1r,5s)-8-(piperidin-4-ylmethyl)-8-azabicyclo[3.2.1]octan-3-yl)-1H-indazol-3-yl)piperidine-2,6-dione

To a stirred solution of tert-butyl 4-(((1r,5s)-3-(3-(2,6-dioxopiperidin-3-yl)-1-methyl-1H-indazol-7-yl)-8-azabicyclo[3.2.1]octan-8-yl)methyl)piperidine-1-carboxylate (0.61 g, 1.11 mmol) in DCM (12.2 mL), TFA (6.1 mL) was added at 0° C. The reaction mixture was allowed to warm to room temperature over 3 hours with stirring. The reaction mixture was concentrated under reduced pressure and co-distilled with DCM (2×10 mL) to obtain a crude product. The crude product was triturated with diethyl ether to obtain 3-(1-methyl-7-((1r,5s)-8-(piperidin-4-ylmethyl)-8-azabicyclo[3.2.1]octan-3-yl)-1H-indazol-3-yl)piperidine-2,6-dione (0.51 g (TFA salt)) as a pale-yellow, gummy solid. LC-MS (ESI): m/z=450.31 [M+H]⁺.

### Preparation of 3-(7-((1r,5s)-8-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-8-azabicyclo[3.2.1]octan-3-yl)-1-methyl-1H-indazol-3-yl)piperidine-2,6-dione

To a stirred solution of 3-(1-methyl-7-((1r,5s)-8-(piperidin-4-ylmethyl)-8-azabicyclo[3.2.1]octan-3-yl)-1H-indazol-3-yl)piperidine-2,6-dione (0.15 g, 0.33 mmol) in DMSO (1.5 mL), DIPEA (1.5 mL) and (S)-2-cyclopropyl-10-((2,5-dichloropyrimidin-4-yl)methyl)-3,3-difluoro-7-methyl-1,2,3,4-tetrahydro-[1,4]oxazepino[2,3-c]quinolin-6(7H)-one (0.06 g, 0.13 mmol) were added. The resulting reaction mixture was stirred at 100° C. for 16 hours. Solvent was removed under vacuum to obtain a crude product. The crude product was purified by prep HPLC to afford 3-(7-((1r,5s)-8-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-8-azabicyclo[3.2.1]octan-3-yl)-1-methyl-1H-indazol-3-yl)piperidine-2,6-dione (0.015 g) as off-white solid. ¹H NMR (400 MHz, DMSO-d6): δ 10.89 (s, 1H), 8.78 (s, 1H), 8.17 (s, 1H), 8.03 (s, 1H), 7.77 (dd, J=1.60, 9.00 Hz, 1H), 7.46 (dd, J=9.20, 17.00 Hz, 2H), 7.25 (d, J=7.20 Hz, 1H), 7.04 (t, J=8.00 Hz, 1H), 6.21 (s, 1H), 4.46-4.32 (m, 5H), 4.15 (s, 3H), 3.89-3.78 (m, 1H), 3.57 (s, 1H), 3.37-3.28 (m, 3H), 2.81 (t, J=12.00 Hz, 2H), 2.65-2.62 (m, 2H), 2.39-2.37 (m, 2H), 2.17-2.13 (m, 3H), 1.96-1.80 (m, 2H), 1.89-1.53 (m, 4H), 1.49 (d, J=7.60 Hz, 2H), 1.39-1.33 (m, 3H), 1.10-1.07 (m, 2H), 0.75-0.63 (m, 1H), 0.50-0.51 (m, 3H), 0.35-0.34 (m, 1H). LC-MS (ESI): m/z=879.54 [M−H]⁻.

### Example 63: 3-(7-(5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)octahydro-2H-pyrrolo[3,4-c]pyridin-2-yl)-1-methyl-1H-indazol-3-yl)piperidine-2,6-dione (Compound 418a)

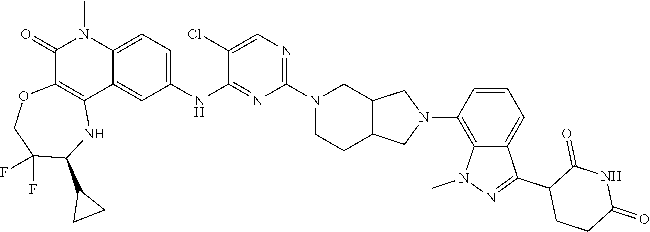 [SMILES: Cn1nc(C2CCC(=O)NC2=O)c2cccc(N3CC4CCN(c5ncc(Cl)c(Nc6ccc7c(c6)c6c(c(=O)n7C)OCC(F)(F)[C@H](C7CC7)N6)n5)CC4C3)c21]

### Preparation of tert-butyl 2-(3-(2,6-bis(benzyloxy)pyridin-3-yl)-1-methyl-1H-indazol-7-yl)octahydro-5H-pyrrolo[3,4-c]pyridine-5-carboxylate

A solution of 3-(2,6-bis(benzyloxy)pyridin-3-yl)-7-bromo-1-methyl-1H-indazole (1.0 g, 2.0 mmol), tert-butyl octahydro-5H-pyrrolo[3,4-c]pyridine-5-carboxylate (0.45 g, 2.0 mmol), and cesium carbonate (1.95 g, 6.0 mmol) in 1,4-dioxane (20 mL) was degassed with argon for 5 minutes. Pd-PEPPSI-IHeptCl (0.97 g, 0.1 mmol) was then added, and the reaction mixture was degassed again for 5 min. The reaction mixture was then heated at 80° C. for 16 h. After that, the reaction mixture was quenched with ice-cold water (50 mL) and extracted with ethyl acetate (3×100 mL). The combined organic layers were washed with brine solution (50 mL) and dried over anhydrous Na₂SO₄, filtered, and dried under vacuum to get a crude product which was then purified by column chromatography (SiO₂; 40% EtOAc in petroleum ether) to afford tert-butyl 2-(3-(2,6-bis(benzyloxy)pyridin-3-yl)-1-methyl-1H-indazol-7-yl)octahydro-5H-pyrrolo[3,4-c]pyridine-5-carboxylate (1.0 g) as a pale brown semi solid.

LC-MS (ESI): m/z=646.97 [M+H]+

### Preparation of tert-butyl 2-(3-(2,6-dioxopiperidin-3-yl)-1-methyl-1H-indazol-7-yl) octahydro-5H-pyrrolo[3,4-c]pyridine-5-carboxylate

To a stirred solution of tert-butyl 2-(3-(2,6-bis(benzyloxy)pyridin-3-yl)-1-methyl-1H-indazol-7-yl)octahydro-5H-pyrrolo[3,4-c]pyridine-5-carboxylate (1.0 g, 1.54 mmol) in THF (60 mL) was added 20% Pd (OH)₂ on carbon (1.0 g,). The reaction mixture was stirred under hydrogen atmosphere (60 psi) at room temperature for 6 h. Upon completion, the reaction mixture was filtered through a celite bed, which was washed with THF and concentrated. The crude residue was washed with n-pentane and dried to afford tert-butyl 2-(3-(2,6-dioxopiperidin-3-yl)-1-methyl-1H-indazol-7-yl) octahydro-5H-pyrrolo[3,4-c]pyridine-5-carboxylate (0.70 g,) as a brown solid which was directly used in the next step without purification.

LC-MS (ESI): m/z=468.30 [M+H]⁺.

### Preparation of 3-(1-methyl-7-(octahydro-2H-pyrrolo[3,4-c]pyridin-2-yl)-1H-indazol-3-yl)piperidine-2,6-dione

A stirred solution of tert-butyl 2-(3-(2,6-dioxopiperidin-3-yl)-1-methyl-1H-indazol-7-yl) octahydro-5H-pyrrolo[3,4-c]pyridine-5-carboxylate (0.70 g, 1.49 mmol) in DCM (14 mL) was cooled to 0° C. TFA (7.0 mL) was then added, and the reaction mixture was stirred at room temperature for 3 h. Upon completion, the reaction mixture was concentrated to a residue which was washed with n-pentane to give crude 3-(1-methyl-7-(octahydro-2H-pyrrolo[3,4-c]pyridin-2-yl)-1H-indazol-3-yl)piperidine-2,6-dione (0.50 g) as a brown gummy solid. LC-MS (ESI): m/z=368.22 [M+H]+

### Preparation of 3-(7-(5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)octahydro-2H-pyrrolo[3,4-c]pyridin-2-yl)-1-methyl-1H-indazol-3-yl)piperidine-2,6-dione

To a solution of 3-(1-methyl-7-(octahydro-2H-pyrrolo[3,4-c]pyridin-2-yl)-1H-indazol-3-yl)piperidine-2,6-dione (0.25 g, 0.68 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.25 g, 0.54 mmol) in DMSO was added N,N-diisopropylethylamine (0.70 g, 5.44 mmol). The reaction mixture was stirred at 100° C. for 16 h. Upon completion, the reaction mixture was poured into ice-cold water and stirred for 15 minutes. The resulting precipitate was filtered and dried to obtain a residue which was purified by prep-HPLC to afford 3-(7-(5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)octahydro-2H-pyrrolo[3,4-c]pyridin-2-yl)-1-methyl-1H-indazol-3-yl)piperidine-2,6-dione (0.132 g) as a brown solid.

LC-MS (ESI): m/z=799.49 [M+H]⁺ 1H NMR (400 MHz, DMSO-d₆): δ 10.90 (s, 1H), 8.77 (d, J=5.20 Hz, 1H), 8.18 (s, 1H), 8.05 (s, 1H), 7.79 (d, J=8.80 Hz, 1H), 7.42 (d, J=9.20 Hz, 1H), 7.33 (d, J=8.00 Hz, 1H), 7.04-6.96 (m, 2H), 6.18-6.15 (m, 1H), 4.32-4.30 (m, 3H), 4.09 (s, 3H), 3.62-3.60 (m, 3H), 3.54 (s, 3H), 3.32-3.22 (m, 3H), 2.94-2.89 (m, 2H), 2.67-2.50 (m, 4H), 2.30-2.16 (m, 2H), 1.95-1.85 (m, 1H), 1.80-1.75 (m, 3H), 0.72-0.60 (m, 1H), 0.55-0.45 (m, 2H), 0.40-0.25 (m, 1H).

### Example 64: 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione (Compound 381b)

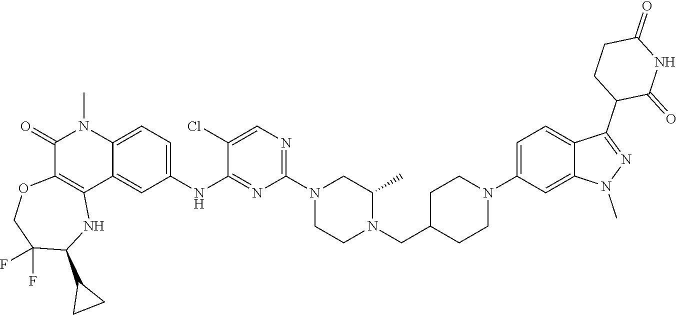 [SMILES: C[C@H]1CN(c2ncc(Cl)c(Nc3ccc4c(c3)c3c(c(=O)n4C)OCC(F)(F)[C@H](C4CC4)N3)n2)CCN1CC1CCN(c2ccc3c(C4CCC(=O)NC4=O)nn(C)c3c2)CC1]

### Preparation of tert-butyl (S)-4-((1-(3-(2,6-bis(benzyloxy)pyridin-3-yl)-1-methyl-1H-indazol-6-yl)piperidin-4-yl)methyl)-3-methylpiperazine-1-carboxylate

To a stirred solution of 3-(2,6-bis(benzyloxy)pyridin-3-yl)-6-bromo-1-methyl-1H-indazole (1.0 g, 1.99 mmol) in dioxane (12.0 mL) was added tert-butyl (S)-3-methyl-4-(piperidin-4-ylmethyl) piperazine-1-carboxylate (1.78 g, 5.99 mmol) and NaOtBu (0.58 g, 5.99 mmol). The reaction mixture was degassed with argon for 5 minutes. Brettphos PdG3 (0.09 g, 0.10 mmol) was then added, and the reaction mixture was heated at 100° C. for 18 h. Upon completion, the reaction mixture was quenched with water (5 mL) and extracted with ethyl acetate (50 mL). The separated organic layers were combined and dried over anhydrous Na₂SO₄, filtered, and dried under vacuum to obtain crude product which was purified by flash column chromatography (3% methanol in DCM as eluent) to obtain tert-butyl (S)-4-((1-(3-(2,6-bis(benzyloxy)pyridin-3-yl)-1-methyl-1H-indazol-6-yl)piperidin-4-yl)methyl)-3-methylpiperazine-1-carboxylate (0.96 g) as a brown semisolid.

LC-MS (ESI): m/z=717.83 [M+H]⁺.

### Preparation of tert-butyl (3S)-4-((1-(3-(2,6-dioxopiperidin-3-yl)-1-methyl-1H-indazol-6-yl)piperidin-4-yl)methyl)-3-methylpiperazine-1-carboxylate

To a stirred solution of tert-butyl (S)-4-((1-(3-(2,6-bis(benzyloxy)pyridin-3-yl)-1-methyl-1H-indazol-6-yl)piperidin-4-yl)methyl)-3-methylpiperazine-1-carboxylate (0.96 g, 1.35 mmol) in THF (38 mL) was added 20% Pd (OH)₂/C (0.96 g, 1.35 mmol). The reaction mixture was stirred under hydrogen atmosphere (80 psi) at room temperature for 16 h. Upon completion, the reaction mixture was diluted with DCM (10 mL), filtered through a celite bed, and washed with 20% THF: DCM (200 mL). The filtrate was collected and concentrated under vacuum to afford crude tert-butyl (3S)-4-((1-(3-(2,6-dioxopiperidin-3-yl)-1-methyl-1H-indazol-6-yl)piperidin-4-yl)methyl)-3-methylpiperazine-1-carboxylate (0.96 g) as a pale brown semi solid which was used in the next step without purification.

LC-MS (ESI): m/z=539.83 [M+H]⁺.

### Preparation of 3-(1-methyl-6-(4-(((S)-2-methylpiperazin-1-yl)methyl)piperidin-1-yl)-1H-indazol-3-yl)piperidine-2,6-dione

To a stirred solution of tert-butyl (3S)-4-((1-(3-(2,6-dioxopiperidin-3-yl)-1-methyl-1H-indazol-6-yl)piperidin-4-yl)methyl)-3-methylpiperazine-1-carboxylate (0.80 g, 1.49 mmol) in DCM (8.0 mL) was added TFA (8.0 mL) at 0° C. The reaction mixture was stirred at room temperature for 3 h. Upon completion, the reaction mixture was concentrated under vacuum to obtain a residue.

This residue was then triturated with petroleum ether (3×10 mL) and dried to afford 3-(1-methyl-6-(4-(((S)-2-methylpiperazin-1-yl)methyl)piperidin-1-yl)-1H-indazol-3-yl)piperidine-2,6-dione (0.80 g) as a pale brown semisolid which was used in the next step without further purification.

LC-MS (ESI): m/z=439.83 [M+H]⁺.

### Preparation of 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione

To a stirred solution of 3-(1-methyl-6-(4-(((S)-2-methylpiperazin-1-yl)methyl)piperidin-1-yl)-1H-indazol-3-yl)piperidine-2,6-dione (0.31 g, 0.70 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.16 g, 0.46 mmol) in DMSO (6.0 mL) was added DIPEA (6.0 mL) and heated at 100° C. for 16 h. Upon completion, the reaction mixture was concentrated under vacuum to obtain a residue which was purified by Prep-HPLC to afford 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-methylpiperazin-1-yl)methyl)piperidin-1-yl)-1-methyl-1H-indazol-3-yl)piperidine-2,6-dione (0.11 g) as a pale brown solid. 1H NMR (400 MHz, DMSO-d₆): δ 10.84 (s, 1H), 8.84 (s, 1H), 8.27 (s, 1H), 8.04 (s, 1H), 7.71 (d, J=10.80 Hz, 1H), 7.45-7.43 (m, 2H), 6.89 (d, J=9.20 Hz, 1H), 6.81 (br s, 1H), 6.21 (s, 1H), 4.43-4.41 (m, 2H), 4.24-4.23 (m, 1H), 3.96 (d, J=12.00 Hz, 2H), 3.88 (s, 3H), 3.77 (d, J=12.40 Hz, 2H), 3.56 (s, 3H), 3.32-3.22 (m, 2H), 2.91-2.83 (m, 1H), 2.80-2.73 (m, 1H), 2.64-2.62 (m, 1H), 2.60-2.59 (m, 2H), 2.30-2.28 (m, 2H), 2.16-2.14 (m, 2H), 2.08 (d, J=8.00 Hz, 1H), 1.91-1.86 (m, 1H), 1.73-1.70 (m, 2H), 1.24-1.21 (m, 4H), 0.95-0.93 (m, 3H), 0.72-0.71 (m, 2H), 0.53-0.51 (m, 2H), 0.32-0.31 (m, 1H).

LC-MS (ESI): m/z=868.44 [M−H]⁻

### Example 65: 3-(6-(8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,8-diazaspiro[5.5]undecan-2-yl)-1-methyl-1H-indazol-3-yl)piperidine-2,6-dione (Compound 401a)

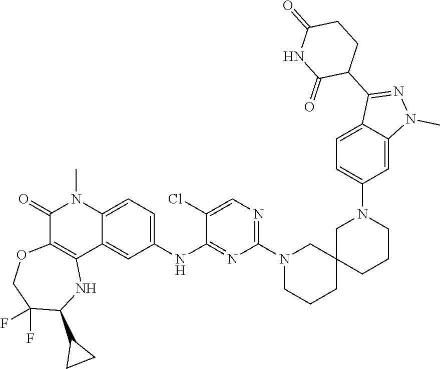 [SMILES: Cn1nc(C2CCC(=O)NC2=O)c2ccc(N3CCCC4(CCCN(c5ncc(Cl)c(Nc6ccc7c(c6)c6c(c(=O)n7C)OCC(F)(F)[C@H](C7CC7)N6)n5)C4)C3)cc21]

### Preparation of tert-butyl 8-(3-(2,6-bis(benzyloxy)pyridin-3-yl)-1-methyl-1H-indazol-6-yl)-2,8-diazaspiro[5.5]undecane-2-carboxylate

To a solution of 3-(2,6-bis(benzyloxy)pyridin-3-yl)-6-bromo-1-methyl-1H-indazole (1.20 g, 2.40 mmol) and tert-butyl 2,8-diazaspiro[5.5]undecane-2-carboxylate (0.91 g, 3.60 mmol) in 1,4-dioxane (24 mL) was added cesium carbonate (2.34 g, 7.20 mmol). The reaction mixture was degassed with argon for 10 minutes. Ruphos (0.22 g, 0.48 mmol) and Ruphos-PdG₃ (0.10 g, 0.12 mmol) were then added, and the reaction mixture was heated at 100° C. for 12 h. Upon completion, the reaction mixture was filtered through a celite bed and concentrated to obtain a residue which was purified by flash column chromatography (30% ethyl acetate in petroleum ether as eluent) to afford tert-butyl 8-(3-(2,6-bis(benzyloxy)pyridin-3-yl)-1-methyl-1H-indazol-6-yl)-2,8-diazaspiro[5.5]undecane-2-carboxylate (1.0 g) as a pale yellow semi-solid.

LC-MS (ESI): m/z=674.78 [M+H]+

### Preparation of tert-butyl 8-(3-(2,6-dioxopiperidin-3-yl)-1-methyl-1H-indazol-6-yl)-2,8-diazaspiro[5.5]undecane-2-carboxylate

To a solution of tert-butyl 8-(3-(2,6-bis(benzyloxy)pyridin-3-yl)-1-methyl-1H-indazol-6-yl)-2,8-diazaspiro[5.5]undecane-2-carboxylate (1.0 g, 1.48 mmol) in THF (20 mL) was added 20% Pd(OH)₂ on carbon (1.0 g, 100%, w/w). The reaction mixture was stirred under hydrogen atmosphere (60 psi) at room temperature for 6 h. Upon completion, the reaction mixture was filtered through a celite bed and concentrated to obtain tert-butyl 8-(3-(2,6-dioxopiperidin-3-yl)-1-methyl-1H-indazol-6-yl)-2,8-diazaspiro[5.5]undecane-2-carboxylate (0.61 g) as a pale brown semi-solid which was used in the next step without purification.

LC-MS (ESI): m/z=496.64 [M+H]⁺.

### Preparation of 3-(1-methyl-7-(2,8-diazaspiro[5.5]undecan-2-yl)-1H-indazol-3-yl)piperidine-2,6-dione

To a solution of tert-butyl 8-(3-(2,6-dioxopiperidin-3-yl)-1-methyl-1H-indazol-6-yl)-2,8-diazaspiro[5.5]undecane-2-carboxylate (0.60 g, 1.21 mmol) in DCM (12 mL) was added

TFA (1.80 mL). The reaction mixture was stirred at rt for 3 h. Upon completion, the reaction mixture was concentrated to obtain 3-(1-methyl-7-(2,8-diazaspiro[5.5]undecan-2-yl)-1H-indazol-3-yl)piperidine-2,6-dione as brown solid (0.53 g) which was used in the next step without purification.

LC-MS (ESI): m/z=396.50 [M+H]⁺.

### Preparation of 3-(6-(8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,8-diazaspiro[5.5]undecan-2-yl)-1-methyl-1H-indazol-3-yl)piperidine-2,6-dione

To a solution of 3-(1-methyl-6-(2,8-diazaspiro[5.5]undecan-2-yl)-1H-indazol-3-yl)piperidine-2,6-dione (0.25 g, 0.63 mmol) in DMSO (5 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.25 mmol) and DIPEA (1.1 mL, 6.32 mmol). The reaction mixture was stirred at 100° C. for 12 h. Upon completion, the reaction mixture was concentrated under reduced pressure to obtain a residue which was purified by prep HPLC, to afford 3-(6-(8-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,8-diazaspiro[5.5]undecan-2-yl)-1-methyl-1H-indazol-3-yl)piperidine-2,6-dione (73 mg) as a pale brown solid.

LC-MS (ESI): m/z=827.51 [M−H]⁻

¹H NMR (400 MHz), DMSO-d₆: δ 10.80 (s, 1H), 8.75-8.69 (m, 1H), 8.33-7.15 (m, 5H), 6.92-6.15 (s, 3H), 4.25-4.22 (m, 3H), 3.93-3.85 (m, 4H), 3.42-3.38 (br, 5H), 3.23-3.20 (br, 6H), 2.67-2.54 (m, 4H), 2.32-2.28 (m, 1H), 2.15-1.90 (m, 1H), 1.52-1.23 (m, 5H), 0.96 (d, J=6.40 Hz, 2H), 0.68-0.49 (m, 4H).

### Example 66: 3-(7-(7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[4.5]decan-2-yl)-1-methyl-1H-indazol-3-yl)piperidine-2,6-dione (Compound 402a)

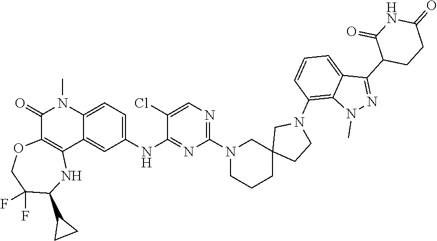 [SMILES: Cn1nc(C2CCC(=O)NC2=O)c2cccc(N3CCC4(CCCN(c5ncc(Cl)c(Nc6ccc7c(c6)c6c(c(=O)n7C)OCC(F)(F)[C@H](C7CC7)N6)n5)C4)C3)c21]

### Preparation of tert-butyl-2-(3-(2,6-bis(benzyloxy)pyridin-3-yl)-1-methyl-1H-indazol-7-yl)-2,7-diazaspiro[4.5]decane-7-carboxylate

A stirred solution of tert-butyl 2,7-diazaspiro[4.5]decane-7-carboxylate (1.0 g, 4.16 mmol), 3-(2,6-bis(benzyloxy)pyridin-3-yl)-7-bromo-1-methyl-1H-indazole (1.6 g, 1.66 mmol) and sodium tert-butoxide (1.2 g, 6.241 mmol) in 1,4-dioxane (20 mL) was degassed with nitrogen for 15 min. Pd-PEPPSI-iHeptCl (0.14 g, 0.104 mmol) was then added. The resulting mixture was degassed again for 10 minutes and heated at 80° C. for 6 h. The reaction mixture was then filtered through a celite bed and concentrated under reduced pressure to give a residue which was purified by flash column chromatography (SiO₂, 100-200 mesh, 10% ethyl acetate in pet-ether) to afford tert-butyl-2-(3-(2,6-bis(benzyloxy)pyridin-3-yl)-1-methyl-1H-indazol-7-yl)-2,7-diazaspiro[4.5]decane-7-carboxylate (0.5 g) as a white solid. LC-MS (ESI): m/z=660.75 [M+H]⁺.

### Preparation of tert-butyl-2-(3-(2,6-dioxopiperidin-3-yl)-1-methyl-1H-indazol-7-yl)-2,7-diazaspiro[4.5]decane-7-carboxylate

To a solution of tert-butyl-2-(3-(2,6-bis(benzyloxy)pyridin-3-yl)-1-methyl-1H-indazol-7-yl)-2,7-diazaspiro[4.5]decane-7-carboxylate (0.5 g, 0.772 mmol) in ethyl acetate (15 mL) was added 20% Pd(OH)₂ on carbon (50% w/w) (0.5 g, w/w) at room temperature. The reaction mixture was stirred for 12 h. After that, the reaction was then filtered through a celite bed, and the filtrate was concentrated under reduced pressure to give a residue. The residue was purified by flash column chromatography (SiO₂, 100-200 mesh, 25% ethyl acetate in pet-ether) to afford tert-butyl-2-(3-(2,6-dioxopiperidin-3-yl)-1-methyl-1H-indazol-7-yl)-2,7-diazaspiro[4.5]decane-7-carboxylate (0.35 g) as an off-white solid.

LC-MS (ESI): m/z=426.49 [M+H]⁺.

### Preparation of 3-(1-methyl-7-(2,7-diazaspiro[4.5]decan-2-yl)-1H-indazol-3-yl)piperidine-2,6-dione

To a solution of tert-butyl-2-(3-(2,6-dioxopiperidin-3-yl)-1-methyl-1H-indazol-7-yl)-2,7-diazaspiro[4.5]decane-7-carboxylate (0.31 g, 0.745 mmol) in DCM (3.1 mL) was added 4M HCl in 1,4-dioxane (1.75 mL) drop-wise. The reaction mixture was stirred at room temperature for 16 h. The reaction mixture was then concentrated under reduced pressure to give a crude solid which was triturated by diethyl ether (5 mL×3) to afford 3-(1-methyl-7-(2,7-diazaspiro[4.5]decan-2-yl)-1H-indazol-3-yl)piperidine-2,6-dione (0.31 g) as a white solid.

LC-MS (ESI): m/z=382.22 [M+H]⁺.

### Preparation of 3-(7-(7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[4.5]decan-2-yl)-1-methyl-1H-indazol-3-yl)piperidine-2,6-dione

To a stirred solution of 3-(1-methyl-7-(2,7-diazaspiro[4.5]decan-2-yl)-1H-indazol-3-yl)piperidine-2,6-dione (0.15 g, 0.393 mmol) in DMSO (5 mL) was added DIPEA (1.07 mL, 3.932 mmol). The reaction mixture was degassed with nitrogen for 1 min followed by addition of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.092 g, 0.197 mmol). The reaction mixture was then stirred and heated to 140° C. for 4 h. The reaction was then quenched with water and a precipitate was observed. The precipitate was collected by filtration and dried to provide a crude solid which was purified by prep-HPLC to afford 3-(7-(7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[4.5]decan-2-yl)-1-methyl-1H-indazol-3-yl)piperidine-2,6-dione (0.044 g) as a white solid.

¹H NMR (400 MHz, DMSO-d6): δ 10.83 (s, 1H), 8.82 (bs, 1H), 8.28 (s, 1H), 8.19 (s, 2H), 7.74-7.70 (m, 1H), 7.35-7.31 (m, 2H), 6.94-6.84 (m, 2H), 6.26-6.20 (m, 1H), 4.32-4.28 (s, 3H), 3.87-3.72 (m, 6H), 3.20 (bs, 9H), 2.67 (bs, 2H), 2.32-2.15 (m, 2H), 1.90-1.86 (m, 1H), 1.68-1.57 (m, 5H), 1.31-1.23 (m, 1H), 0.70 (bs, 1H), 0.50 (bs, 2H), 0.48 (bs, 1H).

LC-MS (ESI): m/z=813.45 [M+H]⁺.

### Example 67: 3-(6-((2-(5-chloro-4-(((S)-2-cyclopropyl-3,3-difluoro-7-methyl-6-oxo-1,2,3,4,6,7-hexahydro-[1,4]-oxazepino[2,3-c]-quinolin-10-yl)amino)pyrimidin-2-yl)octahydro-1H-isoindol-5-yl)amino)-1-methyl-1H-indazol-3-yl)piperidine-2,6-dione (Compound 423a)

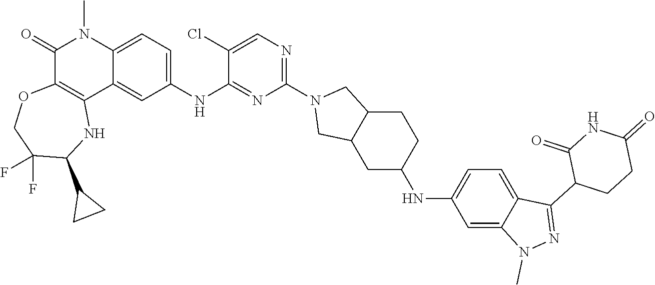 [SMILES: Cn1nc(C2CCC(=O)NC2=O)c2ccc(NC3CCC4CN(c5ncc(Cl)c(Nc6ccc7c(c6)c6c(c(=O)n7C)OCC(F)(F)[C@H](C7CC7)N6)n5)CC4C3)cc21]

### Preparation of tert-butyl-5-((3-(2,6-bis(benzyloxy)pyridin-3-yl)-1-methyl-1H-indazol-6-yl)amino)octahydro-2H-isoindole-2-carboxylate

A stirred solution of 3-(2,6-bis(benzyloxy)pyridin-3-yl)-6-bromo-1-methyl-1H-indazole (1.0 g, 2.0 mmol), tert-butyl 5-aminooctahydro-2H-isoindole-2-carboxylate (0.72 g, 3.0 mmol), and cesium carbonate (1.95 g, 6.0 mmol) in 1,4-dioxane (20 mL) was degassed with argon for 10 minutes. Afterwards, RuPhos (0.09 g, 0.20 mmol) and RuPhos-PdG3 (0.08 g, 0.10 mmol) were added and the mixture was degassed for an additional 5 min, stirred, and heated at 80° C. for 3 h.

The reaction mixture was diluted with ethyl acetate (200 mL) and filtered through a celite bed. The collected filtrate was concentrated under vacuum to give a residue which was purified by flash column chromatography (SiO₂, 230-400, 30% ethyl acetate in petroleum ether) to obtain tert-butyl-5-((3-(2,6-bis(benzyloxy)pyridin-3-yl)-1-methyl-1H-indazol-6-yl)amino)octahydro-2H-isoindole-2-carboxylate (0.70 g) as an off white solid.

LC-MS (ESI): m/z=660.71 [M+H]+

### Preparation of tert-butyl-5-((3-(2,6-dioxopiperidin-3-yl)-1-methyl-1H-indazol-6-yl)amino)octahydro-2H-isoindole-2-carboxylate

To a solution of tert-butyl-5-((3-(2,6-bis(benzyloxy)pyridin-3-yl)-1-methyl-1H-indazol-6-yl)amino)octahydro-2H-isoindole-2-carboxylate (0.70 g, 1.06 mmol) in tetrahydrofuran (28.0 mL) was added 20% Pd(OH)₂ on carbon (0.70 g). The reaction mixture was stirred under hydrogen atmosphere (60 psi) at room temperature for 8 h. The reaction mixture was then filtered through a celite bed and washed with ethyl acetate (300 mL). The collected filtrate was concentrated under vacuum, washed with n-pentane (20 mL), and dried under vacuum to afford tert-butyl-5-((3-(2,6-dioxopiperidin-3-yl)-1-methyl-1H-indazol-6-yl)amino)octahydro-2H-isoindole-2-carboxylate (0.60 g) as a brown solid.

LC-MS (ESI): m/z=482.31 [M+H]⁺.

### Preparation of 3-(1-methyl-6-((octahydro-1H-isoindol-5-yl)amino)-1H-indazol-3-yl)piperidine-2,6-dione

To a solution of tert-butyl-5-((3-(2,6-dioxopiperidin-3-yl)-1-methyl-1H-indazol-6-yl)amino)octahydro-2H-isoindole-2-carboxylate (0.60 g, 1.25 mmol) in DCM (12.0 mL) was added TFA (6.0 mL) at 0° C. The reaction mixture was stirred at room temperature for 3 h. The reaction mixture was concentrated under vacuum, triturated with diethyl ether (30 mL), and dried under vacuum to afford 3-(1-methyl-6-((octahydro-1H-isoindol-5-yl)amino)-1H-indazol-3-yl)piperidine-2,6-dione (0.44 g) as a brown solid.

LC-MS (ESI): m/z=382.48 [M+H]+

### Preparation of 3-(6-((2-(5-chloro-4-(((S)-2-cyclopropyl-3,3-difluoro-7-methyl-6-oxo-1,2,3,4,6,7-hexahydro-[1,4]-oxazepino[2,3-c]-quinolin-10-yl)amino)pyrimidin-2-yl)octahydro-1H-isoindol-5-yl)amino)-1-methyl-1H-indazol-3-yl)piperidine-2,6-dione

To a stirred solution of 3-(1-methyl-6-((octahydro-1H-isoindol-5-yl)amino)-1H-indazol-3-yl)piperidine-2,6-dione (0.25 g, 0.66 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.12 g, 0.26 mmol) in DMSO (2.5 mL) was added N,N-diisopropylethylamine (1.0 mL, 5.25 mmol). The reaction mixture was heated at 100° C. for 4 h. The reaction mixture was concentrated under reduced pressure and purified by Prep-HPLC to afford 3-(6-((2-(5-chloro-4-(((S)-2-cyclopropyl-3,3-difluoro-7-methyl-6-oxo-1,2,3,4,6,7-hexahydro-[1,4]-oxazepino[2,3-c]-quinolin-10-yl)amino)pyrimidin-2-yl)octahydro-1H-isoindol-5-yl)amino)-1-methyl-1H-indazol-3-yl)piperidine-2,6-dione (65 mg) as a brown solid.

¹H NMR (400 MHz, DMSO-d₆): δ 10.88 (s, 1H), 8.65 (br s, 1H), 8.20 (d, J=9.20 Hz, 1H), 8.03 (d, J=4.40 Hz, 1H), 7.87-7.84 (m, 1H), 7.45-7.4 (m, 1H), 7.29 (d, J=8.40 Hz, 1H), 6.45 (d, J=8.00 Hz, 1H), 6.38 (s, 1H), 6.12-6.07 (m, 1H), 5.58-5.56 (m, 1H), 4.37-4.35 (m, 2H), 4.16-4.15 (m, 1H), 3.81 (s, 3H), 3.55 (d, J=13.20 Hz, 4H), 3.45-3.44 (m, 4H), 2.58-2.54 (m, 2H), 2.58-2.54 (m, 1H), 2.14-2.13 (m, 2H), 2.11-1.88 (m, 2H), 1.81-1.75 (m, 2H), 1.36-1.32 (m, 3H), 1.24-1.21 (m, 1H), 0.70-0.69 (m, 2H), 0.51-0.50 (m, 2H), 0.35-0.33 (m, 1H).

LC-MS (ESI): m/z=813.48 [M+H]+

### Example 68: 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)octahydro-1H-isoindol-5-yl)amino)-1-methyl-1H-indazol-3-yl)piperidine-2,6-dione (Compound 424a)

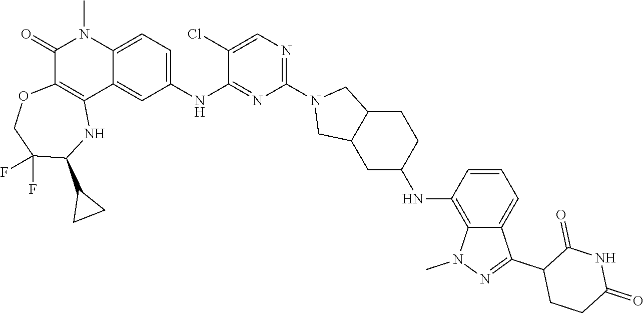 [SMILES: Cn1nc(C2CCC(=O)NC2=O)c2cccc(NC3CCC4CN(c5ncc(Cl)c(Nc6ccc7c(c6)c6c(c(=O)n7C)OCC(F)(F)[C@H](C7CC7)N6)n5)CC4C3)c21]

### Preparation of tert-butyl 5-((3-(2,6-bis(benzyloxy)pyridin-3-yl)-1-methyl-1H-indazol-7-yl)amino)octahydro-2H-isoindole-2-carboxylate

To a stirred solution of 3-(2,6-bis(benzyloxy)pyridin-3-yl)-7-bromo-1-methyl-1H-indazole (1.0 g, 2.0 mmol) and tert-butyl 5-amino octahydro-2H-isoindole-2-carboxylate (0.72 g, 3.0 mmol) in 1,4-dioxane (20 mL) was added cesium carbonate (1.95 g, 6.0 mmol). The reaction mixture degassed with argon for 10 minutes. Afterwards, Pd-PEPPSI-IHeptCl (0.09 g, 0.10 mmol) was added, and the mixture was degassed again for 5 min and stirred at 80° C. for 3 h. The reaction mixture was then cooled to room temperature, filtered through a celite bed, and then the filtrate was concentrated to get a residue which was purified by flash column chromatography (SiO₂, 100-200, 30% ethyl acetate in petroleum ether) to afford tert-butyl 5-((3-(2,6-bis(benzyloxy)pyridin-3-yl)-1-methyl-1H-indazol-7-yl)amino)octahydro-2H-isoindole-2-carboxylate (0.7 g) as an off white solid.

LC-MS (ESI): m/z=660.6 [M+H]+

### Preparation of tert-butyl 5-((3-(2,6-dioxopiperidin-3-yl)-1-methyl-1H-indazol-7-yl)amino)octahydro-2H-isoindole-2-carboxylate

To a solution of tert-butyl 5-((3-(2,6-bis(benzyloxy)pyridin-3-yl)-1-methyl-1H-indazol-7-yl)amino)octahydro-2H-isoindole-2-carboxylate (0.7 g, 1.06 mmol) in tetrahydrofuran (28 mL), 20% Pd(OH)₂ on carbon (0.7 g) was added, and the reaction was stirred at room temperature with hydrogen pressure (60 PSI) for 8 h. The reaction mixture was then filtered through a celite bed, washed with ethyl acetate, and concentrated. The crude residue was washed with n-pentane and dried to afford tert-butyl 5-((3-(2,6-dioxopiperidin-3-yl)-1-methyl-1H-indazol-7-yl)amino)octahydro-2H-isoindole-2-carboxylate (0.60 g) as a brown solid, which was used in the next step without purification.

LC-MS (ESI): m/z=482.3 [M+H]+

### Preparation of 3-(1-methyl-7-((octahydro-1H-isoindol-5-yl)amino)-1H-indazol-3-yl)piperidine-2,6-dione

To a solution of tert-butyl 5-((3-(2,6-dioxopiperidin-3-yl)-1-methyl-1H-indazol-7-yl)amino)octahydro-2H-isoindole-2-carboxylate (0.6 g, 1.25 mmol) in DCM (12 mL) was added

TFA (6 mL) at 0° C. The reaction mixture was stirred at room temperature for 3 h. The reaction mixture was then concentrated to get the crude residue which was triturated with diethyl ether (3 times), decanted, and dried to afford 3-(1-methyl-7-((octahydro-1H-isoindol-5-yl)amino)-1H-indazol-3-yl)piperidine-2,6-dione (0.44 g) as a brown solid.

LC-MS (ESI): m/z=382.19 [M+H]+

### Preparation of 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)octahydro-1H-isoindol-5-yl)amino)-1-methyl-1H-indazol-3-yl)piperidine-2,6-dione

To a solution of 3-(1-methyl-7-((octahydro-1H-isoindol-5-yl)amino)-1H-indazol-3-yl)piperidine-2,6-dione (0.22 g, 0.58 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.23 mmol) in DMSO (3 mL) was added N,N-diisopropylethylamine (0.8 mL, 4.61 mmol), and the reaction mixture was stirred at 100° C. for 4 h. The reaction mixture was concentrated under reduced pressure. The resulting residue was purified by prep-HPLC to afford 3-(7-((2-(5-chloro-4-(((S)-2-cyclopropyl-3,3-difluoro-7-methyl-6-oxo-1,2,3,4,6,7-hexahydro-[1,4]oxazepino [2,3-c]quinolin-10-yl)amino)pyrimidin-2-yl)octahydro-1H-isoindol-5-yl)amino)-1-methyl-1H-indazol-3-yl)piperidine-2,6-dione (65 mg) as an off white solid. ¹H NMR (400 MHz, DMSO-d6): δ 10.83 (s, 1H), 8.67-8.63 (m, 1H), 8.39 (s, 1H), 8.21 (s, 1H), 8.04 (s, 1H), 7.92-7.84 (m, 1H), 7.48-7.39 (m, 1H), 6.95-6.87 (m, 2H), 6.6-6.5 (m,1H), 6.20-6.10 (m, 1H), 4.88-4.84 (m, 1H), 4.5-4.3 (m, 2H), 4.28-4.11 (m, 4H), 3.6-3.39 (m, 8H), 2.67-2.56 (m, 4H), 2.50-2.49 (m, 1H), 2.33-2.13 (m, 2H), 2.07-1.77 (m, 4H), 1.29-1.23 (m, 4H), 0.69 (br s, 2H).

LC-MS (ESI): m/z=813.4 [M+H]⁺.

### Example 69: 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-1-methyl-1H-indazol-3-yl)piperidine-2,6-dione (Compound 406a)

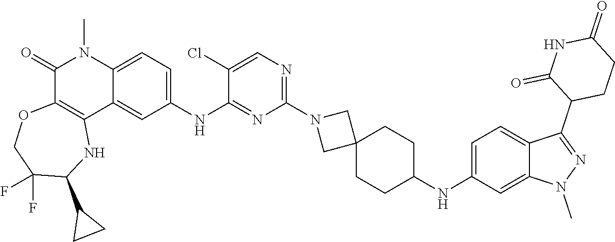 [SMILES: Cn1nc(C2CCC(=O)NC2=O)c2ccc(NC3CCC4(CC3)CN(c3ncc(Cl)c(Nc5ccc6c(c5)c5c(c(=O)n6C)OCC(F)(F)[C@H](C6CC6)N5)n3)C4)cc21]

### Preparation of tert-butyl 7-((3-(2,6-bis(benzyloxy)pyridin-3-yl)-1-methyl-1H-indazol-6-yl)amino)-2-azaspiro[3.5]nonane-2-carboxylate

A stirred solution of 3-(2,6-bis(benzyloxy)pyridin-3-yl)-6-bromo-1-methyl-1H-indazole (0.80 g, 1.60 mmol), tert-butyl-7-amino-2-azaspiro-[3.5]-nonane-2-carboxylate (0.58 g, 2.40 mmol), and KOtBu (0.54 g, 4.80 mmol) in 1,4-dioxane (20 mL) was degassed with argon for 10 minutes.

Afterwards, Brettphos Pd G3 (0.14 g, 0.16 mmol) was added, and the mixture was stirred and heated at 80° C. for 2 h. The reaction mixture was filtered through a celite bed and washed with ethyl acetate (100 mL). The collected filtrate was concentrated under vacuum and purified by flash column chromatography (SiO₂, 230-400, 40% ethyl acetate in petroleum ether) to afford tert-butyl 7-((3-(2,6-bis(benzyloxy)pyridin-3-yl)-1-methyl-1H-indazol-6-yl)amino)-2-azaspiro[3.5]nonane-2-carboxylate (0.90 g) as an off white solid.

LC-MS (ESI): m/z=661.6 [M+2]+

### Preparation of tert-butyl 7-((3-(2,6-dioxopiperidin-3-yl)-1-methyl-1H-indazol-6-yl) amino)-2-azaspiro [3.5]nonane-2-carboxylate

To a solution of tert-butyl 7-((3-(2,6-bis(benzyloxy)pyridin-3-yl)-1-methyl-1H-indazol-6-yl)amino)-2-azaspiro[3.5]nonane-2-carboxylate (0.90 g, 1.36 mmol) in THF (27.0 mL) was added 20% Pd(OH)₂ on carbon (0.90 g). The reaction mixture was stirred under H₂ (70 psi) atmosphere at room temperature for 16 h. The reaction mixture was filtered through a celite bed and washed with ethyl acetate (200 mL). The collected filtrate was concentrated under reduced pressure to afford tert-butyl 7-((3-(2,6-dioxopiperidin-3-yl)-1-methyl-1H-indazol-6-yl) amino)-2-azaspiro [3.5]nonane-2-carboxylate (0.70 g) as brown solid which was used in the next step without further purification. LC-MS (ESI): m/z=482.57 [M+H]⁺.

### Preparation of 3-(6-((2-azaspiro [3.5]nonan-7-yl) amino)-1-methyl-1H-indazol-3-yl) piperidine-2,6-dione

A solution of tert-butyl 7-((3-(2,6-dioxopiperidin-3-yl)-1-methyl-1H-indazol-6-yl) amino)-2-azaspiro [3.5]nonane-2-carboxylate (0.70 g, 1.45 mmol) in DCM (14.0 mL) was cooled to 0° C. TFA (7.0 mL) was added, and the reaction mixture was stirred at room temperature for 3 h. The reaction mixture was concentrated under vacuum, triturated with diethyl ether (30 mL), and dried under vacuum to afford 3-(6-((2-azaspiro [3.5]nonan-7-yl) amino)-1-methyl-1H-indazol-3-yl) piperidine-2,6-dione (0.9 g) as an off white solid.

¹H NMR (400 MHz, DMSO-d₆): δ 10.82 (s, 1H), 8.59 (s, 1H), 7.34 (d, J=8.80 Hz, 1H), 6.53 (d, J=8.00 Hz, 1H), 6.42 (s, 1H), 4.18-4.16 (m, 1H), 3.73-3.72 (m, 3H), 3.70-3.69 (m, 4H), 3.67-3.65 (m, 2H), 2.60-2.59 (m, 2H), 2.28-2.26 (m, 1H), 1.92-1.88 (m, 4H), 1.60 (t, J=10.40 Hz, 2H), 1.24-1.19 (m, 3H).

LC-MS (ESI): m/z=382.4 [M+H]⁺.

### Preparation of 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-1-methyl-1H-indazol-3-yl)piperidine-2,6-dione

To a solution of 3-(6-((2-azaspiro [3.5]nonan-7-yl) amino)-1-methyl-1H-indazol-3-yl) piperidine-2,6-dione (0.25 g, 0.66 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.12 g, 0.26 mmol) in DMSO (5.0 mL) was added N,N-Diisopropylethylamine (0.9 mL, 5.25 mmol). The reaction mixture was stirred at 100° C. for 2 h. The reaction mixture was concentrated under reduced pressure. The resulting residue was purified by prep-HPLC to afford 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-1-methyl-1H-indazol-3-yl)piperidine-2,6-dione (0.058 g) as an off white solid. 1H NMR (400 MHz, DMSO-d₆): δ 10.82 (s, 1H), 8.75 (s, 1H), 8.20 (br s, 1H), 8.03 (s, 1H), 7.86 (d, J=8.40 Hz, 1H), 7.44 (d, J=9.20 Hz, 1H), 7.31 (d, J=8.80 Hz, 1H), 6.50 (d, J=8.80 Hz, 1H), 6.37 (s, 1H), 6.14 (br s, 1H), 5.66 (br s, 1H), 4.45-4.42 (m, 2H), 4.19-4.15 (m, 1H), 3.80 (s, 3H), 3.67-3.65 (m, 3H), 3.57 (s, 3H), 3.32-3.24 (m, 2H), 2.62-3.58 (m, 2H), 2.24-2.15 (m, 2H), 1.90 (d, J=10.00 Hz, 4H), 1.63-1.58 (m, 2H), 1.36-1.32 (m, 3H), 0.74-0.72 (m, 2H), 0.55-0.52 (m, 2H), 0.37-0.35 (m, 1H).

LC-MS (ESI): m/z=813.52 [M+H]⁺

### Example 70. 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-1-methyl-1H-indazol-3-yl)piperidine-2,6-dione (Compound 407a)

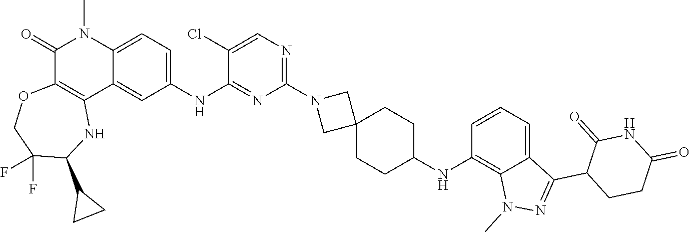 [SMILES: Cn1nc(C2CCC(=O)NC2=O)c2cccc(NC3CCC4(CC3)CN(c3ncc(Cl)c(Nc5ccc6c(c5)c5c(c(=O)n6C)OCC(F)(F)[C@H](C6CC6)N5)n3)C4)c21]

### Preparation of tert-butyl 7-((3-(2,6-bis(benzyloxy)pyridin-3-yl)-1-methyl-1H-indazol-7-yl)amino)-2-azaspiro[3.5]nonane-2-carboxylate

A solution of 3-(2,6-bis(benzyloxy)pyridin-3-yl)-7-bromo-1-methyl-1H-indazole (0.80 g, 1.59 mmol), tert-butyl 7-amino-2-azaspiro[3.5]nonane-2-carboxylate(1.53 g,6.39 mmol), and cesium carbonate (1.56 g, 4.79 mmol) in 1,4-dioxane (16 mL) was degassed with argon gas for 5 minutes.

Afterwards, Pd-PEPPSI-IHeptCl (0.08 g, 0.08 mmol) was added, and the reaction mixture was degassed for another 5 min and stirred at 100° C. for 16 h. The reaction mixture was then diluted with ice-cold water (50 mL) and extracted with ethyl acetate (3×50 mL). The combined organic layers were washed with brine solution (50 mL) and dried over anhydrous Na₂SO₄, filtered, dried under vacuum and purified by column chromatography (SiO₂ (100-200 mesh); 40% EtOAc in petroleum ether) to afford tert-butyl 7-((3-(2,6-bis(benzyloxy)pyridin-3-yl)-1-methyl-1H-indazol-7-yl)amino)-2-azaspiro[3.5]nonane-2-carboxylate (0.70 g) as an off white solid.

LC-MS (ESI): m/z=660.75. [M+H]+

### Preparation of tert-butyl 7-((3-(2,6-dioxopiperidin-3-yl)-1-methyl-1H-indazol-7-yl)amino)-2-azaspiro[3.5]nonane-2-carboxylate

To a solution of tert-butyl 7-((3-(2,6-bis(benzyloxy)pyridin-3-yl)-1-methyl-1H-indazol-7-yl)amino)-2-azaspiro[3.5]nonane-2-carboxylate (0.70 g, 1.00 mmol) in THF (28 mL) was added 20% Pd (OH)₂ on carbon (0.70 g, 100% w/w). The reaction mixture was stirred under hydrogen atmosphere (60 psi) at room temperature for 8 h. The reaction mixture was then filtered through a celite bed, concentrated under reduced pressured, washed with diethyl ether, and dried to afford tert-butyl 7-((3-(2,6-dioxopiperidin-3-yl)-1-methyl-1H-indazol-7-yl)amino)-2-azaspiro[3.5]nonane-2-carboxylate (0.60 g) as an off white solid, which was used in the next step without purification.

LC-MS (ESI): m/z=482.61 [M+H]⁺.

### Preparation of 3-(7-((2-azaspiro[3.5]nonan-7-yl)amino)-1-methyl-1H-indazol-3-yl)piperidine-2,6-dione

A solution of tert-butyl 7-((3-(2,6-dioxopiperidin-3-yl)-1-methyl-1H-indazol-7-yl)amino)-2-azaspiro[3.5]nonane-2-carboxylate (0.60 g, 1.24 mmol) in DCM (12 mL) was cooled to 0° C., and TFA (6 mL) was added. The resulting reaction mixture was stirred at room temperature for 3 h. The reaction mixture was then concentrated under reduced pressure, co-distilled with toluene, and washed with diethyl ether to afford 3-(7-((2-azaspiro[3.5]nonan-7-yl)amino)-1-methyl-1H-indazol-3-yl)piperidine-2,6-dione (0.60 g) as a brown solid.

LC-MS (ESI): m/z=382.48 [M+H]⁺.

### Preparation of 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-1-methyl-1H-indazol-3-yl)piperidine-2,6-dione

To a solution 3-(7-((2-azaspiro[3.5]nonan-7-yl)amino)-1-methyl-1H-indazol-3-yl)piperidine-2,6-dione (0.30 g, 0.78 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.14 g, 0.31 mmol) in DMSO (6 mL) was added N,N-diisopropylethylamine (1 mL, 6.29 mmol). The reaction mixture was stirred at 100° C. for 4 h. The reaction mixture was then poured into ice-cold water and stirred for 15 minutes. The resulting precipitate was filtered, dried, and purified by prep-HPLC to afford 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-1-methyl-1H-indazol-3-yl)piperidine-2,6-dione (0.080 g) as an off white solid. 1H NMR (400 MHz, DMSO-d₆): δ 10.83 (s, 1H), 8.75 (s, 1H), 8.24 (d, J=1.60 Hz, 1H), 8.03 (s, 1H), 7.83 (d, J=9.20 Hz, 1H), 7.44 (d, J=9.20 Hz, 1H), 6.96 (d, J=8.00 Hz, 1H), 6.88-6.86 (m, 2H), 6.52 (d, J=7.60 Hz, 1H), 4.82 (br s, 1H), 4.43-4.42 (m, 2H), 4.24-4.22 (m, 4H), 3.69-3.65 (m, 4H), 3.57 (s, 3H), 3.26-3.23 (m, 2H), 2.60-2.58 (m, 2H), 2.29-2.27 (m, 1H), 2.16-2.15 (m, 1H), 1.91-1.89 (m, 4H), 1.60-1.58 (m, 2H), δ 1.34-1.32 (m, 3H), 0.72-0.71 (m, 1H), 0.53-0.51 (m, 2H), 0.37-0.35 (m, 1H).

LC-MS (ESI): m/z=813.52 [M+H]+

### Example 71: 3-(7-(4-(((2S,6R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-dimethylpiperazin-1-yl)methyl)piperidin-1-yl)-1-methyl-1H-indazol-3-yl)piperidine-2,6-dione (Compound 398a)

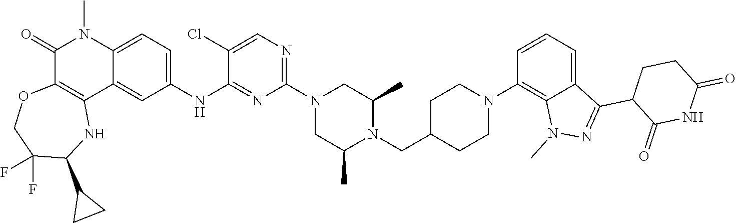 [SMILES: C[C@@H]1CN(c2ncc(Cl)c(Nc3ccc4c(c3)c3c(c(=O)n4C)OCC(F)(F)[C@H](C4CC4)N3)n2)C[C@H](C)N1CC1CCN(c2cccc3c(C4CCC(=O)NC4=O)nn(C)c23)CC1]

### Preparation of tert-butyl (3S,5R)-4-((1-((benzyloxy)carbonyl)piperidin-4-yl)methyl)-3,5-dimethylpiperazine-1-carboxylate

To a stirred solution of tert-butyl (3S,5R)-3,5-dimethylpiperazine-1-carboxylate (6.0 g, 27.99 mmol) and benzyl 4-formylpiperidine-1-carboxylate (8.30 g, 33.59 mmol) in DCM (120 mL) was added acetic acid (1.60 mL, 27.97 mmol). The reaction was stirred for 3 h. Sodium triacetoxyborohydride (11.86 g, 55.99 mmol) was added, and the resulting reaction mixture was stirred at room temperature for 16 h. The reaction mixture was quenched with NH₄Cl (70 mL) and extracted with DCM (2×60 mL). The combined organic layers were washed with brine solution (50 mL), dried over anhydrous Na₂SO₄, filtered, concentrated under reduced pressure to afford the crude product. The crude product was purified by flash chromatography (230-400 silica gel; 25-35% EtOAc in petroleum ether) to afford tert-butyl (3S,5R)-4-((1-((benzyloxy)carbonyl)piperidin-4-yl)methyl)-3,5-dimethylpiperazine-1-carboxylate (2.30 g) as a yellow sticky compound.

LC-MS (ESI): m/z=446.35 [M+H]⁺.

### Preparation of tert-butyl (3S,5R)-3,5-dimethyl-4-(piperidin-4-ylmethyl)piperazine-1-carboxylate

A solution of tert-butyl (3S,5R)-4-((1-((benzyloxy)carbonyl)piperidin-4-yl)methyl)-3,5-dimethylpiperazine-1-carboxylate (2.30 g, 5.16 mmol) in THF (25 mL) was degassed with nitrogen for 5 minutes. 20% Pd(OH)₂ on carbon (1.15 g) was added, and the reaction was put under hydrogen atmosphere (80 psi). The reaction was stirred at room temperature for 16 h. The reaction mixture was diluted with THF (50 mL) and filtered through a celite bed. The celite was washed with THF:DCM (1:1, 300 mL). The filtrate was concentrated and dried to afford tert-butyl (3S, 5R)-3,5-dimethyl-4-(piperidin-4-ylmethyl)piperazine-1-carboxylate (1.44 g) as a brown gummy solid.

¹H NMR (400 MHz, DMSO-d6-D₂O-Ex): δ 3.12 (d, J=11.60 Hz, 2H), 2.65-2.62 (m, 3H), 2.38-2.28 (m, 5H), 1.78 (d, J=12.40 Hz, 4H), 1.39 (s, 11H), 1.11 (q, J=10.80 Hz, 2H), 0.98 (d, J=6.00 Hz, 7H).

### Preparation of tert-butyl (3S, 5R)-4-((1-(3-(2,6-bis(benzyloxy)pyridin-3-yl)-1-methyl-1H-indazol-7-yl)piperidin-4-yl)methyl)-3,5-dimethylpiperazine-1-carboxylate

To a stirred solution of 3-(2,6-bis(benzyloxy)pyridin-3-yl)-7-bromo-1-methyl-1H-indazole (0.70 g, 1.39 mmol) and tert-butyl (3S, 5R)-3,5-dimethyl-4-(piperidin-4-ylmethyl)piperazine-1-carboxylate (1.30 g, 4.17 mmol) in dioxane (14 mL) was added cesium carbonate (1.37 g, 4.20 mmol). The reaction was degassed with argon for 15 minutes. Pd-PEPPSI-IHeptCl (0.06 g, 0.06 mmol) was added, and the reaction was stirred at 100° C. for 16 h. The reaction mixture was filtered through a celite bed, and the celite was washed with ethyl acetate (80 mL). The filtrate was concentrated and dried under vacuum to afford a crude product. The crude product was purified by flash chromatography (230-400 silica gel; 35-45% EtOAc in petroleum ether) to afford tert-butyl (3S, 5R)-4-((1-(3-(2,6-bis(benzyloxy)pyridin-3-yl)-1-methyl-1H-indazol-7-yl)piperidin-4-yl)methyl)-3,5-dimethylpiperazine-1-carboxylate (0.40 g) as a yellow sticky compound.

LC-MS (ESI): m/z=731.50 [M+H]⁺.

### Preparation of tert-butyl (3S, 5R)-4-((1-(3-(2,6-dioxopiperidin-3-yl)-1-methyl-1H-indazol-7-yl)piperidin-4-yl)methyl)-3,5-dimethylpiperazine-1-carboxylate

To a stirred solution of tert-butyl (3S, 5R)-4-((1-(3-(2,6-bis(benzyloxy)pyridin-3-yl)-1-methyl-1H-indazol-7-yl)piperidin-4-yl)methyl)-3,5-dimethylpiperazine-1-carboxylate (0.40 g, 0.54 mmol) in THF (16 mL) was added 20% Pd(OH)₂ on carbon (0.40 g). The resultant reaction mixture was put under hydrogen atmosphere (80 psi) and stirred at room temperature for 5 h. The reaction mixture was filtered through a celite bed, and the celite was washed with THF:DCM (1:1) (80 mL). The filtrate was concentrated and dried to afford tert-butyl (3S, 5R)-4-((1-(3-(2,6-dioxopiperidin-3-yl)-1-methyl-1H-indazol-7-yl)piperidin-4-yl)methyl)-3,5-dimethylpiperazine-1-carboxylate (0.36 g) as a brown gummy solid. LC-MS (ESI): m/z=553.51 [M+H]⁺.

### Preparation of 3-(7-(4-(((2S, 6R)-2,6-dimethylpiperazin-1-yl)methyl)piperidin-1-yl)-1-methyl-1H-indazol-3-yl)piperidine-2,6-dione

A stirred solution of tert-butyl (3S, 5R)-4-((1-(3-(2,6-dioxopiperidin-3-yl)-1-methyl-1H-indazol-7-yl)piperidin-4-yl)methyl)-3,5-dimethylpiperazine-1-carboxylate (0.35 g, 0.68 mmol) in DCM (7 mL) was cooled to 0° C., and TFA (3.50 mL) was added. The reaction was allowed to warm to room temperature over 2 h with stirring. The reaction mixture was concentrated under reduced pressure and co-distilled with DCM (3×5 mL) to obtain a crude product. The crude product was triturated with n-pentane and dried to afford 3-(7-(4-(((2S, 6R)-2,6-dimethylpiperazin-1-yl)methyl)piperidin-1-yl)-1-methyl-1H-indazol-3-yl)piperidine-2,6-dione (0.31 g) as a brown gummy solid.

LC-MS (ESI): m/z=453.59 [M+H]⁺.

### Preparation of 3-(7-(4-(((2S, 6R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-dimethylpiperazin-1-yl)methyl)piperidin-1-yl)-1-methyl-1H-indazol-3-yl)piperidine-2,6-dione

To a stirred solution of 3-(7-(4-(((2S, 6R)-2,6-dimethylpiperazin-1-yl)methyl)piperidin-1-yl)-1-methyl-1H-indazol-3-yl)piperidine-2,6-dione (0.15 g, 0.33 mmol) in DMSO (1.5 mL) were added DIPEA (1.5 mL) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.12 mmol). The reaction was stirred at 100° C. for 6 h. Solvent was removed via under vacuum to provide a crude product. The crude product was purified by prep-HPLC to afford 3-(7-(4-(((2S, 6R)-4-(5-chloro-4-(((S)-2-cyclopropyl-3,3-difluoro-7-methyl-6-oxo-1,2,3,4,6,7-hexahydro-[1,4]oxazepino [2,3-c]quinolin-10-yl)amino)pyrimidin-2-yl)-2,6-dimethylpiperazin-1-yl)methyl)piperidin-1-yl)-1-methyl-1H-indazol-3-yl)piperidine-2,6-dione (0.05 g) as an off-white solid.

¹H NMR (400 MHz, DMSO-d₆): δ 10.87 (s, 1H), 8.86 (s, 1H), 8.19 (d, J=1.60 Hz, 1H), 8.03 (s, 1H), 7.69 (dd, J=2.00, 9.00 Hz, 1H), 7.44 (d, J=8.80 Hz, 1H), 7.36 (t, J=4.00 Hz, 1H), 7.00-6.99 (m, 2H), 6.23 (s, 1H), 4.52-4.30 (m, 3H), 4.23 (s, 5H), 3.57 (s, 3H), 3.27 (br s, 3H), 2.67-2.60 (m, 6H), 2.51-2.50 (m, 4H), 2.30 (d, J=5.20 Hz, 1H), 2.18-2.14 (m, 1H), 1.87 (d, J=11.60 Hz, 2H), 1.51 (s, 1H), 1.35 (d, J=6.80 Hz, 3H), 1.01 (s, 6H), 0.71-0.68 (m, 1H), 0.50 (d, J=6.00 Hz, 2H), 0.33 (t, J=5.60 Hz, 1H).

LC-MS (ESI): m/z=884.56 [M+H]⁺.

### Example 72: 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[4.5]decan-8-yl)amino)-1-methyl-1H-indazol-3-yl)piperidine-2,6-dione (Compound 408a)

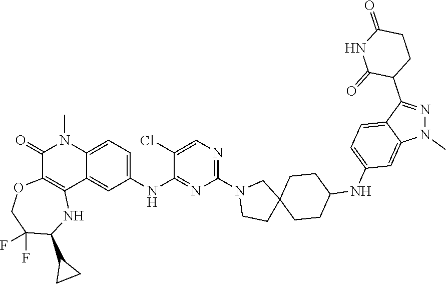 [SMILES: Cn1nc(C2CCC(=O)NC2=O)c2ccc(NC3CCC4(CC3)CCN(c3ncc(Cl)c(Nc5ccc6c(c5)c5c(c(=O)n6C)OCC(F)(F)[C@H](C6CC6)N5)n3)C4)cc21]

### Preparation of tert-butyl 8-((3-(2,6-bis(benzyloxy)pyridin-3-yl)-1-methyl-1H-indazol-6-yl) amino)-2-azaspiro [4,5]decane-2-carboxylate

To a stirred solution of 3-(2,6-bis(benzyloxy)pyridin-3-yl)-6-bromo-1-methyl-1H-indazole (0.80 g, 1.60 mmol) in dioxane (16.0 mL) was added tert-butyl 8-amino-2-azaspiro[4.5]decane-2-carboxylate (0.61 g, 2.40 mmol) and cesium carbonate (1.56 g, 4.80 mmol). The reaction mixture was degassed with argon for 5 minutes. Afterwards, RuPhos (0.07 g, 0.16 mmol) and RuPhos PdG3 (0.07 g, 0.08 mmol) were added, and the mixture was stirred and heated at 100° C. for 18 h. The reaction mixture was diluted with DCM (50 mL), filtered through a celite pad, dried, and purified by column chromatography (SiO₂, 230-400, 21% ethyl acetate in petroleum ether) to afford tert-butyl 8-((3-(2,6-bis(benzyloxy)pyridin-3-yl)-1-methyl-1H-indazol-6-yl) amino)-2-azaspiro [4,5]decane-2-carboxylate (0.85 g) as a pale brown semisolid. ¹H NMR: (400 MHz, DMSO-d₆): δ 7.86 (d, J=8.00 Hz, 1H), 7.46 (d, J=6.80 Hz, 2H), 7.41-7.28 (m, 9H), 6.55 (d, J=8.00 Hz, 1H), 6.37-6.41 (m, 2H), 5.70 (br s, 1H), 5.42 (d, J=10.40 Hz, 4H), 4.03 (d, J=7.20 Hz, 1H), 3.91 (s, 3H), 3.32-3.21 (m, 2H), 3.04-3.13 (m, 1H), 1.90-1.89 (m, 2H), 1.85-1.75 (m, 2H), 1.90-1.99 (m, 2H), 1.44-1.47 (m, 1H), 1.40 (s, 9H), 1.19-1.24 (m, 2H), 1.16-1.17 (m, 2H). LC-MS (ESI): m/z=674.66 [M+H]⁺.

### Preparation of tert-butyl 8-((3-(2,6-dioxopiperidin-3-yl)-1-methyl-1H-indazol-6-yl) amino)-2-azaspiro [4.5]decane-2-carboxylate

To a solution of tert-butyl 8-((3-(2,6-bis(benzyloxy)pyridin-3-yl)-1-methyl-1H-indazol-6-yl) amino)-2-azaspiro [4,5]decane-2-carboxylate (0.85 g, 1.26 mmol) in THF (102 mL) was added 20% Pd(OH)₂ on carbon (0.85 g, 100% w/w). The reaction mixture was stirred under H₂ atmosphere (80 psi) at room temperature for 16 h. The reaction mixture was then diluted with DCM (10 mL), filtered through a celite bed, and washed with DCM:THF (1:1, 500 ml). The collected filtrate was concentrated and dried to obtain tert-butyl 8-((3-(2,6-dioxopiperidin-3-yl)-1-methyl-1H-indazol-6-yl) amino)-2-azaspiro [4.5]decane-2-carboxylate (0.92 g) as a pale brown semi-solid which was used in the next step without further purification.

LC-MS (ESI): m/z=469.65 [M+H]⁺.

### Preparation of 3-(6-((2-azaspiro [4.5]decan-8-yl) amino)-1-methyl-1H-indazol-3-yl) piperidine-2,6-dione

To a solution tert-butyl 8-((3-(2,6-dioxopiperidin-3-yl)-1-methyl-1H-indazol-6-yl) amino)-2-azaspiro [4.5]decane-2-carboxylate (0.92 g, 1.86 mmol) in DCM (10.0 mL), trifluoroacetic acid (7.0 mL) was added. The reaction mixture was stirred at room temperature for 3 h. The reaction mixture was concentrated under vacuum to afford 3-(6-((2-azaspiro [4.5]decan-8-yl) amino)-1-methyl-1H-indazol-3-yl) piperidine-2,6-dione (0.82 g) as a pale green semi-solid which was used in the next step without purification.

LC-MS (ESI): m/z=396.55 [M+H]⁺

### Preparation of 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[4.5]decan-8-yl)amino)-1-methyl-1H-indazol-3-yl)piperidine-2,6-dione

To a stirred solution of 3-(6-((2-azaspiro [4.5]decan-8-yl) amino)-1-methyl-1H-indazol-3-yl) piperidine-2,6-dione (0.30 g, 0.76 mmol) in DMSO (3.0 mL) was added (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.18 g, 0.38 mmol) and DIPEA (3.0 mL, 15.17 mmol). The reaction mixture was heated at 100° C. for 16 h. The reaction mixture was concentrated under reduced pressure and purified by prep-HPLC to afford 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[4.5]decan-8-yl)amino)-1-methyl-1H-indazol-3-yl)piperidine-2,6-dione (0.049 g) as a pale brown solid.

¹H NMR (400 MHz, DMSO-d₆): δ 10.82 (s, 1H), 8.70 (s, 1H), 8.29-8.20 (m, 1H), 8.03 (s, 1H), 7.83 (d, J=8.80 Hz, 1H), 7.44 (d, J=8.80 Hz, 1H), 7.31 (d, J=8.80 Hz, 1H), 6.51-6.49 (m, 1H), 6.38 (br s, 1H), 6.16 (br s, 1H), 5.75-5.71 (m, 1H), 4.35-4.32 (m, 2H), 4.16-4.15 (m, 1H), 3.80 (s, 3H), 3.57 (s, 3H), 3.47-3.39 (m, 4H), 3.23-3.19 (m, 2H), 2.60-2.51 (m, 2H), 2.28-2.26 (m, 1H), 2.24-2.22 (m, 1H), 1.75-1.72 (m, 5H), 1.64-1.58 (m, 2H), 1.50-1.47 (m, 2H), 1.34-1.24 (m, 1H), 0.72-0.71 (m, 2H), 0.52-0.51 (m, 2H), 0.35-0.32 (m, 1H).

LC-MS (ESI): m/z=825.45 [M−H]⁻.

### Example 73: 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-1,4-diazepan-1-yl)methyl)piperidin-1-yl)-1-methyl-1H-indazol-3-yl)piperidine-2,6-dione (Compound 427a)

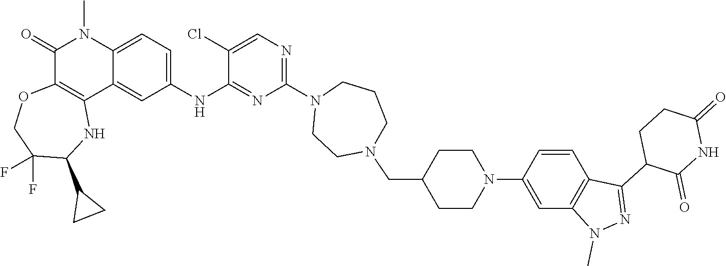 [SMILES: Cn1nc(C2CCC(=O)NC2=O)c2ccc(N3CCC(CN4CCCN(c5ncc(Cl)c(Nc6ccc7c(c6)c6c(c(=O)n7C)OCC(F)(F)[C@H](C7CC7)N6)n5)CC4)CC3)cc21]

### Preparation of (1-(3-(2,6-bis(benzyloxy)pyridin-3-yl)-1-methyl-1H-indazol-6-yl)piperidin-4-yl)methanol

To stirred solution of 3-(2,6-bis(benzyloxy)pyridin-3-yl)-6-bromo-1-methyl-1H-indazole (1.5 g, 2.99 mmol) and piperidin-4-ylmethanol(1.38 g, 1.99 mmol) in dioxane (15 mL) was added cesium carbonate (2.92 g, 8.97 mmol). The reaction mixture was degassed for 15 minutes. Afterwards, Pd-PEPPSI-iHeptCl (0.14 g, 0.14 mmol) was added, and the mixture was stirred at 80° C. for 16 h. The reaction mixture was then cooled to room temperature, filtered through a celite bed, and the filtrate was concentrated to get crude compound as a brown semi-solid which was purified by flash column chromatography (SiO₂, 230-400, 30% ethyl acetate in petroleum ether) to afford (1-(3-(2,6-bis(benzyloxy)pyridin-3-yl)-1-methyl-1H-indazol-6-yl)piperidin-4-yl)methanol (1.5 g) as a green semi-solid.

LC-MS (ESI): m/z=535.57 [M+H]⁺

### Preparation of (1-(3-(2,6-bis(benzyloxy)pyridin-3-yl)-1-methyl-1H-indazol-6-yl)piperidin-4-yl)methyl methanesulfonate

To a solution of (1-(3-(2,6-bis(benzyloxy)pyridin-3-yl)-1-methyl-1H-indazol-6-yl)piperidin-4-yl)methanol (1.5 g, 2.8 mmol) in DCM (15 mL) was added triethylamine (1.56 mL, 11.23 mmol) and methanesulfonyl chloride (0.26 mL, 3.36 mmol) at 0° C. The reaction mixture was then stirred at room temperature for 1 h. The reaction mixture was then quenched with saturated sodium bicarbonate, extracted with EtOAc (3×5 mL). The combined organic layers were dried with sodium sulfate, filtered, and concentrated under vacuum to afford (1-(3-(2,6-bis(benzyloxy)pyridin-3-yl)-1-methyl-1H-indazol-6-yl)piperidin-4-yl)methyl methanesulfonate (1.50 g) as a yellow semi-solid which was used in the next step without further purification.

LC-MS (ESI): m/z=613.62 [M+H]⁺.

### Preparation of tert-butyl 4-((1-(3-(2,6-bis(benzyloxy)pyridin-3-yl)-1-methyl-1H-indazol-6-yl)piperidin-4-yl)methyl)-1,4-diazepane-1-carboxylate

To a solution of (1-(3-(2,6-bis(benzyloxy)pyridin-3-yl)-1-methyl-1H-indazol-6-yl)piperidin-4-yl)methyl methanesulfonate (1.50 g, 2.45 mmol) and tert-butyl 1,4-diazepane-1-carboxylate (0.39 g, 7.35 mmol) in DMSO (15 mL) was added N,N-diisopropylethylamine (4.2 mL, 24.5 mmol). The reaction mixture was stirred at 100° C. for 2 h. Upon completion, cold water was added to the reaction mixture, which was stirred for 5 min. The resulting solid was filtered under vacuum and purified by flash column (SiO₂, 100-200, 10% MeOH in DCM) to afford tert-butyl 4-((1-(3-(2,6-bis(benzyloxy)pyridin-3-yl)-1-methyl-1H-indazol-6-yl)piperidin-4-yl)methyl)-1,4-diazepane-1-carboxylate (0.8 g).

LC-MS (ESI): m/z=717.83 [M+H]⁺.

### Preparation of tert-butyl 4-((1-(3-(2,6-dioxopiperidin-3-yl)-1-methyl-1H-indazol-6-yl)piperidin-4-yl)methyl)-1,4-diazepane-1-carboxylate

To a solution of tert-butyl 4-((1-(3-(2,6-bis(benzyloxy)pyridin-3-yl)-1-methyl-1H-indazol-6-yl)piperidin-4-yl)methyl)-1,4-diazepane-1-carboxylate (0.8 g, 1.11 mmol) in THF (16 mL) and acetic acid (0.4 mL) was added, 20% Pd (OH)₂ on carbon, moisture 50% wet (1.25 g 8.9 mmol). The reaction mixture was stirred under hydrogen atmosphere (80 psi) at room temperature for 16 h. The reaction mixture was diluted with DCM, filtered through a celite bed, and washed with 30% THF in DCM. The collected filtrate was concentrated under vacuum to provide crude tert-butyl 4-((1-(3-(2,6-dioxopiperidin-3-yl)-1-methyl-1H-indazol-6-yl)piperidin-4-yl)methyl)-1,4-diazepane-1-carboxylate (0.49 g) as a light yellow gummy which was used in the next step without further purification.

LC-MS (ESI): m/z=539.41 [M+H]⁺.

### Preparation of 3-(6-(4-((1,4-diazepan-1-yl)methyl)piperidin-1-yl)-1-methyl-1H-indazol-3-yl)piperidine-2,6-dione

To a solution of tert-butyl 4-((1-(3-(2,6-dioxopiperidin-3-yl)-1-methyl-1H-indazol-6-yl)piperidin-4-yl)methyl)-1,4-diazepane-1-carboxylate (0.49 g, 0.91 mmol) in DCM (9.8 mL) was added TFA (4.9 mL). The reaction mixture was stirred at room temperature for 2 h. The reaction mixture was then concentrated and triturated with diethyl ether to afford 3-(6-(4-((1,4-diazepan-1-yl)methyl)piperidin-1-yl)-1-methyl-1H-indazol-3-yl)piperidine-2,6-dione (0.40 g) as a TFA salt which was used in the next step without further purification.

LC-MS (ESI): m/z=439.35 [M+H]⁺.

### Preparation of 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-1,4-diazepan-1-yl)methyl)piperidin-1-yl)-1-methyl-1H-indazol-3-yl)piperidine-2,6-dione

To a solution of 3-(6-(4-((1,4-diazepan-1-yl)methyl)piperidin-1-yl)-1-methyl-1H-indazol-3-yl)piperidine-2,6-dione (0.2 g, 0.45 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.085 g, 0.18 mmol) in DMSO (4 mL) was added N,N-diisopropylethylamine (0.81 mL, 4.56 mmol). The reaction mixture was stirred at 100° C. for 6 h. The reaction mixture was quenched with ice-cold water, and the solid was filtered and purified by prep HPLC to afford 3-(6-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-1,4-diazepan-1-yl)methyl)piperidin-1-yl)-1-methyl-1H-indazol-3-yl)piperidine-2,6-dione as pale brown solid (0.017 g).

¹H NMR (400 MHz, DMSO-d₆): δ 10.84 (s, 1H), 8.74 (s, 1H), 8.19 (s, 1H), 8.03 (s, 1H), 7.80-7.77 (d, J=10.80 Hz, 1H), 7.47-7.41 (m, 2H), 6.88-6.86 (m, 1H), 6.79 (s, 1H), 6.18 (s, 1H), 4.45-4.41 (m, 2H), 4.26-4.22 (m, 1H), 3.88 (s, 3H), 3.72-3.32 (m, 10H), 2.67-2.51 (m, 6H), 2.33-2.28 (m, 2H), 2.18-2.13 (m, 1H), 1.73-1.68 (s, 7H), 1.31-1.30 (m, 1H), 1.23-1.18 (m, 3H), 0.72-0.70 (m, 1H), 0.51 (t, J=11.20 Hz, 2H), 0.35-0.34 (m, 1H).

LC-MS (ESI): m/z=870.48 [M+H]⁺.

### Example 74: 3-(6-(3-(4-(5-chloro-4-(((S)-2-cyclopropyl-3,3-difluoro-7-methyl-6-oxo-1,2,3,4,6,7-hexahydro-[1,4]-oxazepino[2,3-c]quinolin-10-yl)amino)pyrimidin-2-yl)piperazin-1-yl)piperidin-1-yl)-1-methyl-1H-indazol-3-yl)piperidine-2,6-dione (Compound 428a)

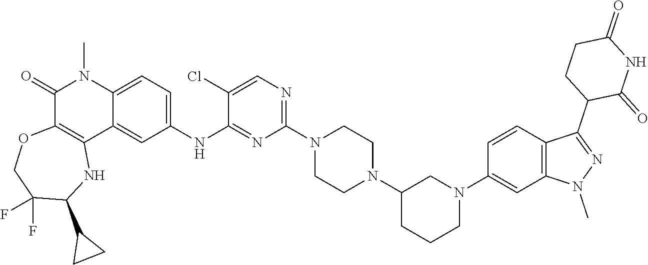 [SMILES: Cn1nc(C2CCC(=O)NC2=O)c2ccc(N3CCCC(N4CCN(c5ncc(Cl)c(Nc6ccc7c(c6)c6c(c(=O)n7C)OCC(F)(F)[C@H](C7CC7)N6)n5)CC4)C3)cc21]

### Preparation of tert-butyl 4-(1-(3-(2,6-bis(benzyloxy)pyridin-3-yl)-1-methyl-1H-indazol-6-yl) piperidin-3-yl) piperazine-1-carboxylate

A stirred solution of 3-(2,6-bis(benzyloxy)pyridin-3-yl)-6-bromo-1-methyl-1H-indazole (0.50 g, 1.0 mmol), tert-butyl 4-(piperidin-3-yl) piperazine-1-carboxylate (0.40 g, 1.50 mmol), and cesium carbonate (0.98 g, 3.0 mmol) in 1,4-dioxane (10 mL) was degassed with argon for 10 minutes.

Afterwards, RuPhos (0.05 g, 0.01 mmol) and RuPhos-PdG3 (0.04 g, 0.05 mmol) were added, and the mixture was heated at 80° C. for 6 h. The reaction mixture was diluted with ethyl acetate (200 mL) and filtered through celite bed. The collected filtrate was concentrated under vacuum and purified by flash column chromatography (SiO₂, 230-400 mesh, 40% ethyl acetate in petroleum ether) to afford tert-butyl 4-(1-(3-(2,6-bis(benzyloxy)pyridin-3-yl)-1-methyl-1H-indazol-6-yl) piperidin-3-yl) piperazine-1-carboxylate (0.58 g) as an off white solid.

LC-MS (ESI): m/z=689.74 [M+H]⁺

### Preparation of tert-butyl 4-(1-(3-(2,6-dioxopiperidin-3-yl)-1-methyl-1H-indazol-6-yl) piperidin-3-yl) piperazine-1-carboxylate

To a solution of tert-butyl 4-(1-(3-(2,6-bis(benzyloxy)pyridin-3-yl)-1-methyl-1H-indazol-6-yl) piperidin-3-yl) piperazine-1-carboxylate (0.48 g, 0.70 mmol) and AcOH (0.48 mL) in THF (24 mL) was added 10% Pd/C (0.48 g). The reaction mixture stirred under H₂ (Parr-20 psi) atmosphere for 16 h. The reaction mixture was filtered through a celite bed and washed with ethyl acetate (200 mL). The collected filtrate was concentrated under reduced pressure to afford tert-butyl 4-(1-(3-(2,6-dioxopiperidin-3-yl)-1-methyl-1H-indazol-6-yl) piperidin-3-yl) piperazine-1-carboxylate (0.35 g) as an off-white solid which was used in the next step without further purification.

LC-MS (ESI): m/z=511.62 [M+H]⁺

### Preparation of 3-(1-methyl-6-(3-(piperazin-1-yl) piperidin-1-yl)-1H-indazol-3-yl) piperidine-2,6-dione

To a solution tert-butyl 4-(1-(3-(2,6-dioxopiperidin-3-yl)-1-methyl-1H-indazol-6-yl) piperidin-3-yl) piperazine-1-carboxylate (0.25 g, 0.49 mmol) in DCM (5 mL) was added TFA (2 mL) at 0° C.

The reaction mixture was stirred at room temperature for 3 h. The reaction mixture was concentrated under reduced pressure, triturated with diethyl ether (3 times), and dried under vacuum to afford 3-(1-methyl-6-(3-(piperazin-1-yl) piperidin-1-yl)-1H-indazol-3-yl) piperidine-2,6-dione (0.4 g) as an off white solid.

LC-MS (ESI): m/z=411.52 [M+H]⁺

### Preparation of 3-(6-(3-(4-(5-chloro-4-(((S)-2-cyclopropyl-3,3-difluoro-7-methyl-6-oxo-1,2,3,4,6,7-hexahydro-[1,4]-oxazepino[2,3-c]quinolin-10-yl)amino)pyrimidin-2-yl)piperazin-1-yl)piperidin-1-yl)-1-methyl-1H-indazol-3-yl)piperidine-2,6-dione

To a solution of 3-(1-methyl-6-(3-(piperazin-1-yl) piperidin-1-yl)-1H-indazol-3-yl) piperidine-2,6-dione (0.40 g, 0.98 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.18 g, 0.39 mmol) in DMSO (8 mL) was added N,N-Diisopropylethylamine (1.4 mL, 7.80 mmol). The reaction mixture was stirred at 100° C. for 2 h. The reaction mixture was then concentrated under reduced pressure, and the resulting residue was purified by Prep-HPLC to afford 3-(6-(3-(4-(5-chloro-4-(((S)-2-cyclopropyl-3,3-difluoro-7-methyl-6-oxo-1,2,3,4,6,7-hexahydro-[1,4]-oxazepino[2,3-c]quinolin-10-yl)amino)pyrimidin-2-yl)piperazin-1-yl)piperidin-1-yl)-1-methyl-1H-indazol-3-yl)piperidine-2,6-dione (47 mg) as an off white solid.

¹H NMR (400 MHz, DMSO-d₆): δ 10.84 (s, 1H), 8.85 (s, 1H), 8.23 (s, 1H), 8.05 (s, 1H), 7.69 (d, J=8.80 Hz, 1H), 7.45 (m, 2H), 6.88 (d, J=9.20 Hz, 1H), 6.82 (s, 1H), 6.23 (s, 1H), 4.45-4.42 (m, 2H), 4.41-4.38 (m, 1H), 3.88 (s, 3H), 3.81 (d, J=11.20 Hz, 1H), 3.71 (d, J=11.20 Hz, 1H), 3.56-3.51 (m, 6H), 3.22-3.19 (m, 1H), 2.67-2.61 (m, 8H), 2.33-2.28 (m, 1H), 2.16-2.14 (m, 1H), 1.92-1.91 (m, 1H), 1.81-1.78 (m, 1H), 1.68-1.66 (m, 1H), 1.36-1.33 (m, 3H), 0.69-0.65 (m, 2H), 0.51-0.50 (m, 2H), 0.37-0.35 (m, 1H).

LC-MS (ESI): m/z=842.48 [M+H]⁺

### Example 75: 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione (Compound 429a)

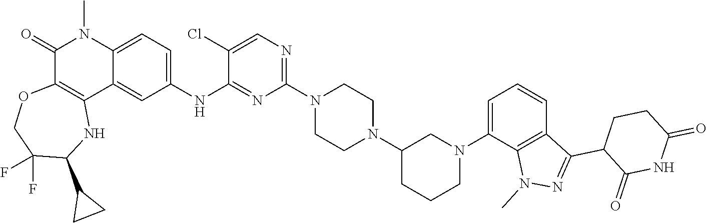 [SMILES: Cn1nc(C2CCC(=O)NC2=O)c2cccc(N3CCCC(N4CCN(c5ncc(Cl)c(Nc6ccc7c(c6)c6c(c(=O)n7C)OCC(F)(F)[C@H](C7CC7)N6)n5)CC4)C3)c21]

### Preparation of tert-butyl 4-(1-(3-(2,6-bis(benzyloxy)pyridin-3-yl)-1-methyl-1H-indazol-7-yl) piperidin-3-yl) piperazine-1-carboxylate

A stirred solution of 3-(2,6-bis(benzyloxy)pyridin-3-yl)-7-bromo-1-methyl-1H-indazole (0.50 g, 1.0 mmol), tert-butyl 4-(piperidin-3-yl) piperazine-1-carboxylate (0.46 g, 1.50 mmol), and cesium carbonate (0.98 g, 3.0 mmol) in 1,4-dioxane (10.0 mL) was degassed with argon for 10 minutes.

Afterwards, Pd-PEPPSI-IHeptCl (0.10 g, 0.10 mmol) was added, and the mixture was heated at 80° C. for 6 h. The reaction mixture was diluted with ethyl acetate (200 mL) and filtered through a celite bed. The collected filtrate was concentrated under reduced pressure and purified by flash column chromatography (SiO₂, 230-400 mesh, 40% ethyl acetate in petroleum ether) to afford tert-butyl 4-(1-(3-(2,6-bis(benzyloxy)pyridin-3-yl)-1-methyl-1H-indazol-7-yl) piperidin-3-yl) piperazine-1-carboxylate (0.50 g) as an off white solid.

LC-MS (ESI): m/z=689.52 [M+H]⁺

### Preparation of tert-butyl 4-(1-(3-(2,6-dioxopiperidin-3-yl)-1-methyl-1H-indazol-7-yl) piperidin-3-yl) piperazine-1-carboxylate

To a solution of tert-butyl 4-(1-(3-(2,6-bis(benzyloxy)pyridin-3-yl)-1-methyl-1H-indazol-7-yl) piperidin-3-yl) piperazine-1-carboxylate (0.45 g, 0.65 mmol) and AcOH (0.45 mL) in THF (27.0 mL) was added 20% Pd(OH)₂ on carbon (0.45 g). The reaction mixture was stirred at room temperature under H₂ (Parr-70 psi) atmosphere for 16 h. The reaction mixture was then filtered through a celite bed and washed with ethyl acetate (300 mL). The collected filtrate was concentrated under reduced pressure, washed with n-pentane (30 mL), and dried under vacuum to afford tert-butyl 4-(1-(3-(2,6-dioxopiperidin-3-yl)-1-methyl-1H-indazol-7-yl) piperidin-3-yl) piperazine-1-carboxylate (0.30 g) as a brown solid.

LC-MS (ESI): m/z=511.66 [M+H]⁺.

### Preparation of 3-(1-methyl-7-(3-(piperazin-1-yl) piperidin-1-yl)-1H-indazol-3-yl) piperidine-2,6-dione

To a solution tert-butyl 4-(1-(3-(2,6-dioxopiperidin-3-yl)-1-methyl-1H-indazol-7-yl) piperidin-3-yl) piperazine-1-carboxylate (0.30 g, 0.59 mmol) in DCM (6 mL) was added TFA (3 mL) at 0° C.

The reaction mixture was stirred at room temperature for 3 h. The reaction mixture was concentrated under vacuum, triturated with diethyl ether (30 mL), and dried under reduced pressure to afford 3-(1-methyl-7-(3-(piperazin-1-yl) piperidin-1-yl)-1H-indazol-3-yl) piperidine-2,6-dione (0.40 g) as an off white solid.

LC-MS (ESI): m/z=411.28 [M+H]⁺.

### Preparation of 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione

To a stirred solution of 3-(1-methyl-7-(3-(piperazin-1-yl) piperidin-1-yl)-1H-indazol-3-yl) piperidine-2,6-dione (0.40 g, 1.01 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.18 g, 0.39 mmol) in DMSO (4.0 mL) was added N,N-diisopropylethylamine (1.40 mL, 7.80 mmol).

The reaction mixture was heated at 100° C. for 2 h. The reaction mixture was concentrated under reduced pressure and purified by Prep-HPLC to afford 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)piperidin-1-yl)-1-methyl-1H-indazol-3-yl)piperidine-2,6-dione (63 mg) as an off white solid.

¹H NMR (400 MHz, DMSO-d₆): δ 10.88 (s, 1H), 8.84 (s, 1H), 8.21 (s, 1H), 8.03 (s, 1H), 7.67 (d, J=7.60 Hz, 1H), 7.42 (d, J=8.80 Hz, 1H), 7.37 (d, J=7.60 Hz, 1H), 7.01-7.00 (m, 2H), 6.20 (br s, 1H), 4.32-4.31 (m, 2H), 4.21 (s, 3H), 3.55 (s, 8H), 3.19-3.17 (m, 3H), 2.57-2.54 (m, 8H), 2.33-2.33 (m, 1H), 2.31-2.28 (m, 1H), 2.15-2.13 (m, 1H), 1.82-1.74 (m, 2H), 1.32-1.31 (m, 2H), 0.69-0.67 (m, 2H), 0.59-0.56 (m, 2H), 0.47-0.46 (m, 1H).

LC-MS (ESI): m/z=842.44 [M+H]⁺

### Example 76: 3-(6-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl) pyrrolidin-1-yl)-1-methyl-1H-indazol-3-yl)piperidine-2,6-dione (Compound 430a)

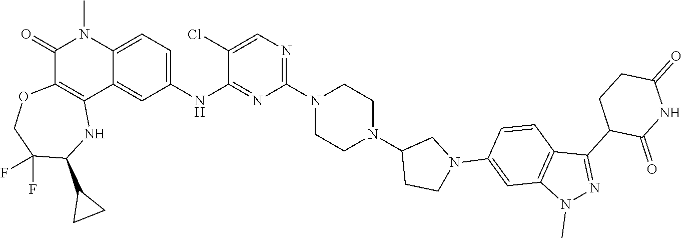 [SMILES: Cn1nc(C2CCC(=O)NC2=O)c2ccc(N3CCC(N4CCN(c5ncc(Cl)c(Nc6ccc7c(c6)c6c(c(=O)n7C)OCC(F)(F)[C@H](C7CC7)N6)n5)CC4)C3)cc21]

### Preparation of tert-butyl 4-(1-(3-(2,6-bis(benzyloxy)pyridin-3-yl)-1-methyl-1H-indazol-6-yl) pyrrolidin-3-yl) piperazine-1-carboxylate

A stirred solution of 3-(2,6-bis(benzyloxy)pyridin-3-yl)-6-bromo-1-methyl-1H-indazole (0.50 g, 1.0 mmol), tert-butyl 4-(pyrrolidin-3-yl)piperazine-1-carboxylate (0.38 g, 1.50 mmol), and cesium carbonate (0.98 g, 3.0 mmol) in 1,4-dioxane (10 mL) was degassed with argon for 10 minutes.

Afterwards, RuPhos (0.05 g, 0.10 mmol) and RuPhos-PdG3 (0.04 g, 0.05 mmol) were added, and the reaction mixture was heated at 80° C. for 6 h. The reaction mixture was then cooled to room temperature, filtered through a celite bed, and washed with ethyl acetate (300 mL). The collected filtrate was then concentrated under reduced pressure and purified by flash column chromatography (SiO₂, 230-400 mesh, 40% Ethyl acetate in petroleum ether) to afford tert-butyl 4-(1-(3-(2,6-bis(benzyloxy)pyridin-3-yl)-1-methyl-1H-indazol-6-yl) pyrrolidin-3-yl) piperazine-1-carboxylate (0.58 g) as an off white solid.

LCMS (ESI): m/z=675.48 [M+H]⁺.

### Preparation of tert-butyl 4-(1-(3-(2,6-dioxopiperidin-3-yl)-1-methyl-1H-indazol-6-yl) pyrrolidin-3-yl) piperazine-1-carboxylate

To a solution of tert-butyl 4-(1-(3-(2,6-bis(benzyloxy)pyridin-3-yl)-1-methyl-1H-indazol-6-yl) pyrrolidin-3-yl) piperazine-1-carboxylate (0.30 g, 0.45 mmol) and AcOH (0.3 mL) in THF (30 mL) was added 20% Pd(OH)₂ on carbon (0.3 0 g). The reaction mixture was stirred at room temperature under H₂ (70 psi) atmosphere for 16 h. The reaction mixture was filtered through a celite bed and washed with ethyl acetate (300 mL). The collected filtrate was concentrated under reduced pressure, washed with n-pentane (30 mL), and dried under vacuum to afford tert-butyl 4-(1-(3-(2,6-dioxopiperidin-3-yl)-1-methyl-1H-indazol-6-yl) pyrrolidin-3-yl) piperazine-1-carboxylate (0.21 g) as a brown solid.

LCMS (ESI): m/z=497.60 [M+H]⁺.

### Preparation of 3-(1-methyl-6-(3-(piperazin-1-yl) pyrrolidin-1-yl)-1H-indazol-3-yl) piperidine-2,6-dione

To a solution tert-butyl 4-(1-(3-(2,6-dioxopiperidin-3-yl)-1-methyl-1H-indazol-6-yl) pyrrolidin-3-yl) piperazine-1-carboxylate (0.21 g, 0.42 mmol) in DCM (4 mL) was added TFA (2 mL) at 0° C.

The reaction mixture was stirred at room temperature for 3 h. The reaction mixture was concentrated under reduced pressure, triturated with diethyl ether (30 mL), and dried under vacuum to afford 3-(1-methyl-6-(3-(piperazin-1-yl) pyrrolidin-1-yl)-1H-indazol-3-yl) piperidine-2,6-dione (0.40 g) as a brown solid.

LCMS (ESI): m/z=397.47 [M+H]⁺.

### Preparation of 3-(6-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl) pyrrolidin-1-yl)-1-methyl-1H-indazol-3-yl)piperidine-2,6-dione

To a stirred solution of 3-(1-methyl-6-(3-(piperazin-1-yl) pyrrolidin-1-yl)-1H-indazol-3-yl) piperidine-2,6-dione (0.25 g, 0.63 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.12 g, 0.25 mmol) in DMSO (5.0 mL) was added N,N-Diisopropylethylamine (0.8 mL, 5.05 mmol) and heated to 100° C. for 2 h. The reaction mixture was concentrated under reduce pressure and purified by prep-HPLC to afford 3-(6-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl) pyrrolidin-1-yl)-1-methyl-1H-indazol-3-yl)piperidine-2,6-dione (30 mg) as an off white solid. ¹H NMR data: (400 MHz, DMSO-d₆): δ 10.83 (s, 1H), 8.87 (s, 1H), 8.24 (s, 1H), 8.06 (s, 1H), 7.71 (d, J=9.20 Hz, 1H), 7.45-7.43 (m, 2H), 6.55 (d, J=8.80 Hz, 1H), 6.37 (s, 1H), 6.24 (s, 1H), 4.45-4.42 (m, 2H), 4.22-4.21 (m, 1H), 3.84 (s, 3H), 3.62-3.57 (m, 4H), 3.54 (s, 3H), 3.46-3.44 (m, 1H), 3.23-3.21 (m, 1H), 3.14-3.12 (m, 1H), 2.94-2.92 (m, 1H), 2.60-2.59 (m, 2H), 2.48-2.47 (m, 4H), 2.18-2.16 (m, 3H), 1.36-1.33 (m, 3H), 0.70-0.67 (m, 2H), 0.52-0.50 (m, 2H), 0.37-0.35 (m, 1H).

LCMS (ESI): m/z=828.42 [M+H]⁺

### Example 77: 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)pyrrolidin-1-yl)-1-methyl-1H-indazol-3-yl)piperidine-2,6-dione (Compound 431a)

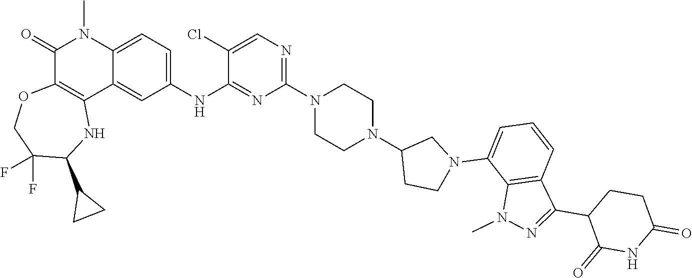 [SMILES: Cn1nc(C2CCC(=O)NC2=O)c2cccc(N3CCC(N4CCN(c5ncc(Cl)c(Nc6ccc7c(c6)c6c(c(=O)n7C)OCC(F)(F)[C@H](C7CC7)N6)n5)CC4)C3)c21]

### Preparation of tert-butyl 4-(1-(3-(2,6-bis(benzyloxy)pyridin-3-yl)-1-methyl-1H-indazol-7-yl) pyrrolidin-3-yl) piperazine-1-carboxylate

A stirred solution of 3-(2,6-bis(benzyloxy)pyridin-3-yl)-7-bromo-1-methyl-1H-indazole (0.50 g, 1.0 mmol), tert-butyl 4-(pyrrolidin-3-yl)piperazine-1-carboxylate (0.43 g, 1.70 mmol), and cesium carbonate (0.98 g, 3.0 mmol) in 1,4-dioxane (10 mL) was degassed with argon for 10 minutes.

Afterwards, Pd-PEPPSI-iHeptCl (0.10 g, 0.10 mmol) was added, and the resulting mixture was heated at 80° C. for 6 h. The reaction mixture was then diluted with ethyl acetate (300 mL) and filtered through a bed of Celite. The filtrate was concentrated under reduced pressure to obtain crude material that was purified by flash silica gel column chromatography (40% ethyl acetate in petroleum ether) to afford tert-butyl 4-(1-(3-(2,6-bis(benzyloxy)pyridin-3-yl)-1-methyl-1H-indazol-7-yl) pyrrolidin-3-yl) piperazine-1-carboxylate (0.50 g) as an off white solid.

LCMS (ESI): m/z=675.48 [M+H]⁺

### Preparation of tert-butyl 4-(1-(3-(2,6-dioxopiperidin-3-yl)-1-methyl-1H-indazol-7-yl) pyrrolidin-3-yl) piperazine-1-carboxylate

To a stirred solution of tert-butyl 4-(1-(3-(2,6-bis(benzyloxy)pyridin-3-yl)-1-methyl-1H-indazol-7-yl) pyrrolidin-3-yl) piperazine-1-carboxylate (0.45 g, 0.67 mmol) and AcOH (0.45 mL) in THF (45 mL) was added 20% Pd(OH)₂ on carbon (0.45 g). The resulting mixture was stirred at room temperature under an H₂ (70 psi) atmosphere for 16 h. The reaction mixture was filtered through a bed of celite and washed with ethyl acetate (400 mL). The filtrate was concentrated under reduced pressure to obtain a crude product that was triturated with n-pentane (30 mL). The solids were filtered and dried under vacuum to afford tert-butyl 4-(1-(3-(2,6-dioxopiperidin-3-yl)-1-methyl-1H-indazol-7-yl) pyrrolidin-3-yl) piperazine-1-carboxylate (0.30 g) as a brown solid.

LCMS (ESI): m/z=497.64 [M+H]⁺

### Preparation of 3-(1-methyl-7-(3-(piperazin-1-yl) pyrrolidin-1-yl)-1H-indazol-3-yl) piperidine-2,6-dione

To a solution tert-butyl 4-(1-(3-(2,6-dioxopiperidin-3-yl)-1-methyl-1H-indazol-7-yl) pyrrolidin-3-yl) piperazine-1-carboxylate (0.25 g, 0.50 mmol) in DCM (5 mL) was added TFA (2.5 mL). The mixture was stirred at ambient temperature for 3 h. The reaction mixture was concentrated under vacuum to obtain crude product that was triturated with diethyl ether (30 mL), filtered, and dried under reduced pressure to afford 3-(1-methyl-7-(3-(piperazin-1-yl) pyrrolidin-1-yl)-1H-indazol-3-yl) piperidine-2,6-dione (0.35 g) as an off-white solid. LCMS (ESI): m/z=397.27 [M+H]⁺.

### Preparation of 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)pyrrolidin-1-yl)-1-methyl-1H-indazol-3-yl)piperidine-2,6-dione

To a stirred solution of 3-(1-methyl-7-(3-(piperazin-1-yl) pyrrolidin-1-yl)-1H-indazol-3-yl) piperidine-2,6-dione (0.30 g, 0.76 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.14 g, 0.30 mmol) in DMSO (3.0 mL) was added N,N-diisopropylethylamine (1.0 mL, 6.06 mmol).

The resulting mixture was stirred and heated at 100° C. for 2 h, cooled to ambient temperature, and then purified by prep-HPLC to afford 3-(7-(3-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)pyrrolidin-1-yl)-1-methyl-1H-indazol-3-yl)piperidine-2,6-dione (96 mg) as an off white solid.

¹H NMR (400 MHz, DMSO-d₆): δ 10.87 (s, 1H), 8.85 (s, 1H), 8.22 (d, J=2.00 Hz, 1H), 8.05 (s, 1H), 7.70 (dd, J=2.00, 8.80 Hz, 1H), 7.43 (d, J=9.20 Hz, 1H), 7.35 (d, J=8.00 Hz, 1H), 7.00-6.98 (m, 2H), 6.23 (s, 1H), 4.32-4.31 (m, 3H), 4.16 (s, 3H), 3.60 (br s, 3H), 3.56 (s, 3H), 3.23-3.21 (m, 3H), 3.06-3.04 (m, 3H), 2.62-2.61 (m, 2H), 2.49-2.44 (m, 3H), 2.43-2.42 (m, 1H), 2.15-2.14 (m, 2H), 1.81-1.71 (m, 1H), 1.36-1.31 (m, 2H), 0.71-0.69 (m, 2H), 0.53-0.50 (m, 2H), 0.35-0.34 (m, 1H).

LCMS (ESI): m/z=828.49 [M+H]⁺

### Example 78: 3-(7-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-oxa-7,9-diazabicyclo[3.3.1]nonan-9-yl)methyl)piperidin-1-yl)-1-methyl-1H-indazol-3-yl)piperidine-2,6-dione (Compound 434a)

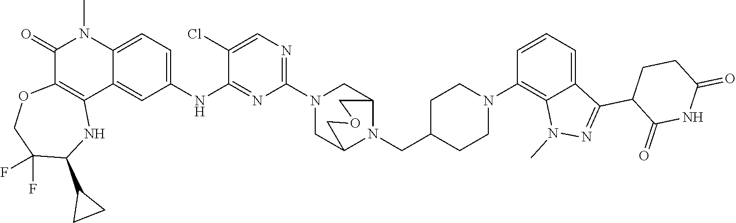 [SMILES: Cn1nc(C2CCC(=O)NC2=O)c2cccc(N3CCC(CN4C5COCC4CN(c4ncc(Cl)c(Nc6ccc7c(c6)c6c(c(=O)n7C)OCC(F)(F)[C@H](C7CC7)N6)n4)C5)CC3)c21]

### Preparation of tert-butyl 9-((1-((benzyloxy)carbonyl)piperidin-4-yl)methyl)-3-oxa-7,9-diazabicyclo[3.3.1]nonane-7-carboxylate

To a solution of tert-butyl 3-oxa-7,9-diazabicyclo[3.3.1]nonane-7-carboxylate (1.0 g, 4.38 mmol) and benzyl 4-formylpiperidine-1-carboxylate (0.97 g, 3.94 mmol) in DCM (20.0 mL, 20 V) was added acetic acid (0.25 mL, 4.38 mmol). The reaction mixture was stirred for 1 h. Afterwards, it was treated with sodium triacetoxyborohydride (1.85 g, 8.76 mmol) at 0° C. The resultant reaction mixture was stirred at room temperature for 16 h. The reaction mixture was then quenched with NH₄Cl solution (30 mL) and extracted with DCM (2×40 mL). The combined organic layers were washed with brine solution (20 mL), dried over anhydrous Na₂SO₄, filtered, and concentrated under reduced pressure to afford tert-butyl 9-((1-((benzyloxy)carbonyl)piperidin-4-yl)methyl)-3-oxa-7,9-diazabicyclo[3.3.1]nonane-7-carboxylate (1.35 g) as a pale yellow semi-solid which was used in the next step without further purification.

LCMS (ESI): m/z=460.60 [M+H]⁺

### Preparation of tert-butyl 9-(piperidin-4-ylmethyl)-3-oxa-7,9-diazabicyclo[3.3.1]nonane-7-carboxylate

To a solution of tert-butyl 9-((1-((benzyloxy)carbonyl)piperidin-4-yl)methyl)-3-oxa-7,9-diazabicyclo[3.3.1]nonane-7-carboxylate (1.30 g, 2.82 mmol) in THF (78.0 mL) was added 20% Pd(OH)₂ on carbon (0.65 g, 50% W/W). The reaction mixture was stirred under hydrogen atmosphere (80 psi) at room temperature for 5 h. The reaction mixture was diluted with THF (50 mL), filtered through a celite bed, and washed with 200 mL of THF:DCM (1:1). The filtrate was concentrated and dried to afford tert-butyl 9-(piperidin-4-ylmethyl)-3-oxa-7,9-diazabicyclo[3.3.1]nonane-7-carboxylate as a black semi-solid (0.97 g) which was used in the next step without further purification.

LCMS (ESI): m/z=326.24 [M+H]⁺

### Preparation of tert-butyl 9-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-7-carboxylate

To a stirred solution of 3-(2,6-bis(benzyloxy)pyridin-3-yl)-7-bromo-1-methyl-1H-indazole (1.0 g, 2.00 mmol) and tert-butyl 9-(piperidin-4-ylmethyl)-3-oxa-7,9-diazabicyclo[3.3.1]nonane-7-carboxylate (0.97 g, 3.00 mmol) in 1,4-dioxane (706.0 mL) was added cesium carbonate (1.95 g, 6.00 mmol). The reaction mixture was degassed with argon for 15 minutes, treated with Pd-PEPPSI-IHeptCl (0.10 g, 0.10 mmol), and purged again for 2 minutes. The resulting reaction mixture was stirred at 100° C. for 16 h. The reaction mixture was cooled to room temperature, diluted with ethyl acetate (40 mL), filtered through a celite bed, and washed with 100 mL of 10% MeOH in DCM. The filtrate was concentrated and purified by flash chromatography (230-400 silica gel; 35% EtOAc in petroleum ether) to afford product tert-butyl 9-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-7-carboxylate as a pale yellow solid (0.48 g) as a pale yellow solid.

LCMS (ESI): m/z=745.87 [M+H]⁺

### Preparation of tert-butyl 9-((1-(3-(2,6-dioxopiperidin-3-yl)-1-methyl-1H-indazol-7-yl)piperidin-4-yl)methyl)-3-oxa-7,9-diazabicyclo[3.3.1]nonane-7-carboxylate

To a solution of tert-butyl 9-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-7-carboxylate (0.48 g, 0.64 mmol) in THF (24 mL) was added 20% Pd(OH)₂ on carbon (0.50 g, 100% w/w). The reaction mixture was stirred under hydrogen atmosphere (80 psi) at room temperature for 16 h. The reaction mixture was then diluted with THF (40 mL) and filtered through a celite bed and washed with THF:DCM (1:1, 100 mL). The filtrate was concentrated and dried to obtain tert-butyl 9-((1-(3-(2,6-dioxopiperidin-3-yl)-1-methyl-1H-indazol-7-yl)piperidin-4-yl)methyl)-3-oxa-7,9-diazabicyclo[3.3.1]nonane-7-carboxylate (0.50 g) as a black solid which was used in the next step without further purification.

LCMS (ESI): m/z=567.68 [M+H]⁺.

### Preparation of 3-(7-(4-((3-oxa-7,9-diazabicyclo[3.3.1]nonan-9-yl)methyl)piperidin-1-yl)-1-methyl-1H-indazol-3-yl)piperidine-2,6-dione

To a solution of tert-butyl 9-((1-(3-(2,6-dioxopiperidin-3-yl)-1-methyl-1H-indazol-7-yl)piperidin-4-yl)methyl)-3-oxa-7,9-diazabicyclo[3.3.1]nonane-7-carboxylate (0.50 g, 0.88 mmol) in DCM (10.0 mL) was added TFA (2.5 mL) at 0° C. The reaction mixture was stirred at room temperature for 3 h. The reaction mixture was concentrated under reduced pressure, co-distilled with DCM (2×10 mL), and triturated with diethyl ether (2×15 mL) to afford 3-(7-(4-((3-oxa-7,9-diazabicyclo[3.3.1]nonan-9-yl)methyl)piperidin-1-yl)-1-methyl-1H-indazol-3-yl)piperidine-2,6-dione as a pale yellow semi-solid (0.35 g).

LCMS (ESI): m/z=467.32 [M+H]⁺.

### Preparation of 3-(7-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-oxa-7,9-diazabicyclo[3.3.1]nonan-9-yl)methyl)piperidin-1-yl)-1-methyl-1H-indazol-3-yl)piperidine-2,6-dione

To a solution of 3-(7-(4-((3-oxa-7,9-diazabicyclo[3.3.1]nonan-9-yl)methyl)piperidin-1-yl)-1-methyl-1H-indazol-3-yl)piperidine-2,6-dione (0.16 g, 0.34 mmol) in DMSO (1.6 mL, 10 V) was added DIPEA (1.6 mL, 10 V) and (S)-2-cyclopropyl-10-((2,5-dichloropyrimidin-4-yl)methyl)-3,3-difluoro-7-methyl-1,2,3,4-tetrahydro-[1,4]oxazepino[2,3-c]quinolin-6(7H)-one (0.08 g, 0.17 mmol). The mixture was then stirred at 100° C. for 16 h. The reaction mixture was then concentrated and purified by Prep-HPLC to afford 3-(7-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-oxa-7,9-diazabicyclo[3.3.1]nonan-9-yl)methyl)piperidin-1-yl)-1-methyl-1H-indazol-3-yl)piperidine-2,6-dione (0.039 g) as an off white solid.

¹H NMR (400 MHz, DMSO-d6): δ10.89 (s, 1H), 8.75 (s, 1H), 8.26 (s, 1H), 8.04 (s, 1H), 7.76 (s, 1H), 7.44 (d, J=9.20 Hz, 1H), 7.36 (d, J=2.00 Hz, 1H), 7.03-6.98 (m, 2H), 6.24 (d, J=17.60 Hz, 1H), 4.44-4.24 (m, 7H), 4.05-3.95 (m, 1H), 3.77-3.66 (m, 4H), 3.56 (s, 3H), 3.37-3.23 (m, 5H), 2.69-2.62 (m, 8H), 2.33-2.14 (m, 2H), 1.89 (d, J=8.00 Hz, 2H), 1.63-1.33 (m, 4H), 0.75-0.65 (m, 1H), 0.51-0.49 (m, 2H), 0.38-0.35 (m, 1H).

LCMS (ESI): m/z=898.59 [M+H]⁺

### Example 79: 3-(7-(4-(((1R, 5S, 6R)-3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azabicyclo[3.1.0]hexan-6-yl)methyl)piperazin-1-yl)-1-methyl-1H-indazol-3-yl)piperidine-2,6-dione (Compound 435a)

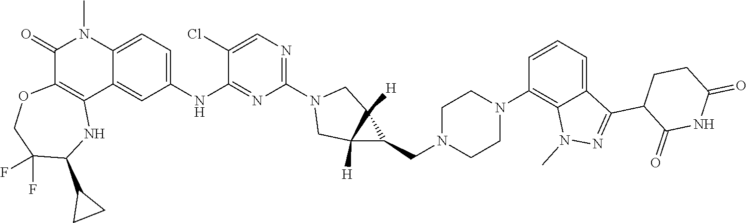 [SMILES: Cn1nc(C2CCC(=O)NC2=O)c2cccc(N3CCN(C[C@@H]4[C@H]5CN(c6ncc(Cl)c(Nc7ccc8c(c7)c7c(c(=O)n8C)OCC(F)(F)[C@H](C8CC8)N7)n6)C[C@@H]45)CC3)c21]

### Preparation of tert-butyl (1R, 5S, 6s)-6-((4-((benzyloxy)carbonyl)piperazin-1-yl)methyl)-3-azabicyclo[3.1.0]hexane-3-carboxylate

To a stirred solution of tert-butyl (1R, 5S, 6r)-6-formyl-3-azabicyclo[3.1.0]hexane-3-carboxylate (2.0 g, 9.46 mmol) and benzyl piperazine-1-carboxylate (2.08 g, 9.44 mmol) in DCE (40 mL) was added acetic acid (0.54 mL, 9.32 mmol). The reaction mixture was stirred for 1 h, treated with sodium triacetoxyborohydride (4.01 g, 18.92 mmol), and stirred at room temperature for 16 h. The reaction mixture was quenched with ammonium chloride solution (70 mL) and extracted with DCM (2×80 mL). The combined organic layer was washed with brine (50 mL), dried over anhydrous Na₂SO₄, filtered, and concentrated under reduced pressure to afford tert-butyl (1R, 5S, 6s)-6-((4-((benzyloxy)carbonyl)piperazin-1-yl)methyl)-3-azabicyclo[3.1.0]hexane-3-carboxylate as a pale yellow sticky compound (3.5 g) which was used in the next step without further purification.

LCMS (ESI): m/z=416.32 [M+H]⁺.

### Preparation of tert-butyl (1R, 5S, 6s)-6-(piperazin-1-ylmethyl)-3-azabicyclo[3.1.0]hexane-3-carboxylate

To a solution of tert-butyl (1R, 5S, 6s)-6-((4-((benzyloxy)carbonyl)piperazin-1-yl)methyl)-3-azabicyclo[3.1.0]hexane-3-carboxylate (3.50 g, 6.76 mmol) in THF (105.0 mL, 30 V) was added 20% Pd(OH)₂ on carbon (1.75 g, 50% w/w). The reaction mixture was stirred under hydrogen atmosphere (80 psi) at room temperature for 16 h. The reaction mixture was diluted with THF (50 mL), filtered through a celite bed, and washed with 200 mL of THF:DCM (1:1). The filtrate was concentrated and dried to afford tert-butyl (1R, 5S, 6s)-6-(piperazin-1-ylmethyl)-3-azabicyclo[3.1.0]hexane-3-carboxylate as a pale yellow gummy compound (2.10 g) which was used in the next step without further purification.

¹H NMR (400 MHz, DMSO-d6): δ 3.40-3.34 (m, 2H), 3.39-3.28 (m, 2H), 2.73 (t, J=4.80 Hz, 4H), 2.49 (s, 4H), 2.20 (d, J=6.80 Hz, 2H), 1.37-1.34 (m, 12H), 0.54-0.46 (m, 1H).

### Preparation of tert-butyl (1R, 5S, 6s)-6-((4-(3-(2,6-bis(benzyloxy)pyridin-3-yl)-1-methyl-1H-indazol-7-yl)piperazin-1-yl)methyl)-3-azabicyclo[3.1.0]hexane-3-carboxylate

To a stirred solution of 3-(2,6-bis(benzyloxy)pyridin-3-yl)-7-bromo-1-methyl-1H-indazole (0.70 g, 1.39 mmol) and tert-butyl (1R, 5S, 6s)-6-(piperazin-1-ylmethyl)-3-azabicyclo[3.1.0]hexane-3-carboxylate (1.18 g, 4.19 mmol) in 1,4-dioxane (14.0 mL, 20 V) was added cesium carbonate (1.36 g, 4.19 mmol). The reaction mixture was degassed with argon for 15 minutes, treated with Pd-PEPPSI-IHeptCl (0.068 g, 0.07 mmol), and stirred at 100° C. for 16 h. The reaction mixture was diluted with ethyl acetate (50 mL), filtered through a celite bed, and washed with 10% MeOH:DCM (250 mL). The filtrate was concentrated, dried under vacuum, and purified by flash chromatography (230-400 silica gel; 30-35% EtOAc in petroleum ether) to afford tert-butyl (1R, 5S, 6s)-6-((4-(3-(2,6-bis(benzyloxy)pyridin-3-yl)-1-methyl-1H-indazol-7-yl)piperazin-1-yl)methyl)-3-azabicyclo[3.1.0]hexane-3-carboxylate (0.51 g) as a yellow sticky compound.

LCMS (ESI): m/z=701.79 [M+H]⁺

### Preparation of tert-butyl (1R, 5S, 6s)-6-((4-(3-(2,6-dioxopiperidin-3-yl)-1-methyl-1H-indazol-7-yl)piperazin-1-yl)methyl)-3-azabicyclo[3.1.0]hexane-3-carboxylate

To a solution of tert-butyl (1R, 5S, 6s)-6-((4-(3-(2,6-bis(benzyloxy)pyridin-3-yl)-1-methyl-1H-indazol-7-yl)piperazin-1-yl)methyl)-3-azabicyclo[3.1.0]hexane-3-carboxylate (0.51 g, 0.72 mmol) in THF (20.0 mL) was added 20% Pd(OH)₂ on carbon (0.51 g, 100% w/w). The reaction mixture was stirred under hydrogen atmosphere (80 psi) at room temperature for 5 h. Afterwards, the reaction mixture was diluted with THF (50 mL), filtered through a celite bed, and washed with THF:DCM (1:1, 150 mL). The filtrate was concentrated and dried to afford tert-butyl (1R, 5S, 6s)-6-((4-(3-(2,6-dioxopiperidin-3-yl)-1-methyl-1H-indazol-7-yl)piperazin-1-yl)methyl)-3-azabicyclo[3.1.0]hexane-3-carboxylate as a pale brown solid (0.41 g) which was used in the next step without further purification.

LCMS (ESI): m/z=523.67 [M+H]⁺

### Preparation of 3-(7-(4-(((1R, 5S, 6r)-3-azabicyclo[3.1.0]hexan-6-yl)methyl)piperazin-1-yl)-1-methyl-1H-indazol-3-yl)piperidine-2,6-dione

To a solution of tert-butyl (1R, 5S, 6s)-6-((4-(3-(2,6-dioxopiperidin-3-yl)-1-methyl-1H-indazol-7-yl)piperazin-1-yl)methyl)-3-azabicyclo[3.1.0]hexane-3-carboxylate (0.41 g, 0.78 mmol) in DCM (4.0 mL) was added TFA (2.1 mL) at 0° C. The reaction mixture was stirred at room temperature for 3 h. The reaction mixture was concentrated under reduced pressure, co-distilled with DCM (3×10 mL), and dried to afford 3-(7-(4-(((1R, 5S, 6r)-3-azabicyclo[3.1.0]hexan-6-yl)methyl)piperazin-1-yl)-1-methyl-1H-indazol-3-yl)piperidine-2,6-dione (0.51 g) as an off white solid which was used in the next step without further purification.

LCMS (ESI): m/z=423.28 [M+H]⁺.

### Preparation of 3-(7-(4-(((1R, 5S, 6R)-3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azabicyclo[3.1.0]hexan-6-yl)methyl)piperazin-1-yl)-1-methyl-1H-indazol-3-yl)piperidine-2,6-dione

To a solution of 3-(7-(4-(((1R, 5S, 6r)-3-azabicyclo[3.1.0]hexan-6-yl)methyl)piperazin-1-yl)-1-methyl-1H-indazol-3-yl)piperidine-2,6-dione (0.25 g, 0.59 mmol) in DMSO (2.5 mL, 10 V) and DIPEA (2.0 mL, 8 V) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.11 g, 0.23 mmol). The reaction mixture was stirred at 100° C. for 16 h. The reaction mixture was concentrated under reduced pressure and purified by Prep-HPLC to afford 3-(7-(4-(((1R, 5S, 6R)-3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azabicyclo[3.1.0]hexan-6-yl)methyl)piperazin-1-yl)-1-methyl-1H-indazol-3-yl)piperidine-2,6-dione (0.09 g) as an off white solid.

¹H NMR (400 MHz, DMSO-d6): δ 10.87 (s, 1H), 8.70 (s, 1H), 8.19 (s, 1H), 8.02 (s, 1H), 7.83 (d, J=9.20 Hz, 1H), 7.44 (d, J=8.80 Hz, 1H), 7.40-7.37 (m, 2H), 7.02 (s, 2H), 6.15 (br s, 1H), 4.45-4.31 (m, 3H), 4.22 (s, 3H), 3.78-3.62 (m, 2H), 3.56 (s, 3H), 3.42-3.40 (m, 4H), 3.17-2.85 (m, 8H), 2.67-2.56 (m, 1H), 2.33 (br s, 3H), 2.18-2.14 (m, 1H), 1.49-1.31 (m, 2H), 0.77-0.63 (m, 2H), 0.53 (br s, 2H), 0.38 (br s, 1H).

LCMS (ESI): m/z=854.57 [M+H]⁺.

### Example 80: 3-(7-(4-((4-(4,5-dichloro-6-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (Compound 436a)

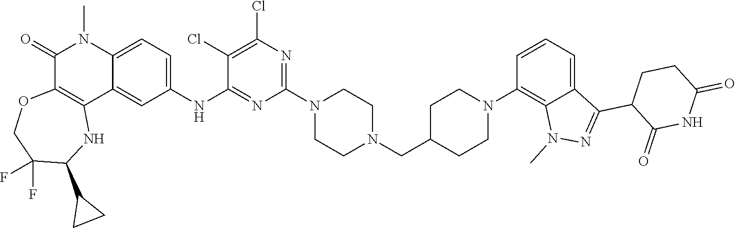 [SMILES: Cn1nc(C2CCC(=O)NC2=O)c2cccc(N3CCC(CN4CCN(c5nc(Cl)c(Cl)c(Nc6ccc7c(c6)c6c(c(=O)n7C)OCC(F)(F)[C@H](C7CC7)N6)n5)CC4)CC3)c21]

### Preparation of (S)-2-cyclopropyl-3,3-difluoro-7-methyl-10-((2,5,6-trichloropyrimidin-4-yl)amino)-1,2,3,4-tetrahydro-[1,4]oxazepino[2,3-c]quinolin-6(7H)-one

To a stirred solution of (S)-10-amino-2-cyclopropyl-3,3-difluoro-7-methyl-1,2,3,4-tetrahydro-[1,4]oxazepino[2,3-c]quinolin-6(7H)-one (0.20 g, 0.62 mmol) in DMSO (2.0 mL) was added perchloropyrimidine (0.08 g, 0.37 mmol) and DIPEA (0.34 mL, 1.86 mmol). The reaction mixture was heated to 100° C. for 3 h. The reaction mixture was concentrated under reduced pressure and purified by Prep-HPLC to afford (S)-2-cyclopropyl-3,3-difluoro-7-methyl-10-((2,5,6-trichloropyrimidin-4-yl)amino)-1,2,3,4-tetrahydro-[1,4]oxazepino[2,3-c]quinolin-6(7H)-one as an off white solid (0.15 g).

LCMS (ESI): m/z=502.43 [M+H]⁺

### Preparation of 3-(7-(4-((4-(4,5-dichloro-6-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione

To a stirred solution of (S)-2-cyclopropyl-3,3-difluoro-7-methyl-10-((2,5,6-trichloropyrimidin-4-yl)amino)-1,2,3,4-tetrahydro-[1,4]oxazepino[2,3-c]quinolin-6(7H)-one (0.15 g, 0.29 mmol) in DMSO (2.0 mL) was added 3-(1-methyl-7-(4-(piperazin-1-ylmethyl)piperidin-1-yl)-1H-indazol-3-yl)piperidine-2,6-dione (0.07 g, 0.17 mmol) and DIPEA (0.34 mL, 1.86 mmol). The reaction mixture was heated to 100° C. for 3 h. The reaction mixture was concentrated under reduced pressure and washed with n-pentane to afford 3-(7-(4-((4-(4,5-dichloro-6-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)piperidine-2,6-dione (0.005 g).

¹H NMR (400 MHz, DMSO-d₆): δ 10.88 (s, 1H), 9.23 (s, 1H), 8.44 (s, 1H), 8.07 (s, 1H), 7.55 (d, J=8.80 Hz, 1H), 7.45 (d, J=9.20 Hz, 1H), 7.37 (d, J=4.40 Hz, 1H), 7.01 (d, J=5.20 Hz, 2H), 6.21 (s, 1H), 4.41-4.43 (m, 3H), 4.25 (s, 3H), 3.57 (s, 7H), 3.27 (s, 3H), 2.50-2.58 (m, 6H), 2.33-2.31 (m, 6H), 1.91-1.88 (m, 2H), 1.73 (s, 1H), 1.31-1.21 (m, 4H), 0.52 (s, 2H), 0.34 (s, 1H).

LCMS (ESI): m/z=890.53 [M+H]⁺.

### Example 81: 4-(((S)-2-cyclopropyl-3,3-difluoro-7-methyl-6-oxo-1,2,3,4,6,7-hexahydro-[1,4]oxazepino[2,3-c]quinolin-10-yl)amino)-2-(4-((1-(3-(2,6-dioxopiperidin-3-yl)-1-methyl-1H-indazol-7-yl)piperidin-4-yl)methyl)piperazin-1-yl)pyrimidine-5-carbonitrile (Compound 437a)

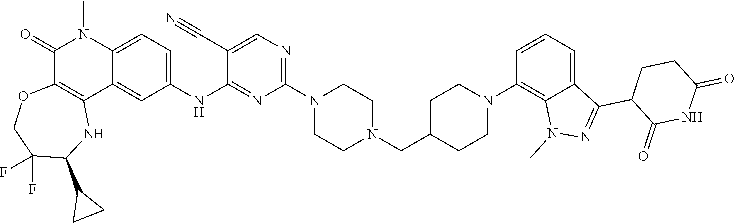 [SMILES: Cn1nc(C2CCC(=O)NC2=O)c2cccc(N3CCC(CN4CCN(c5ncc(C#N)c(Nc6ccc7c(c6)c6c(c(=O)n7C)OCC(F)(F)[C@H](C7CC7)N6)n5)CC4)CC3)c21]

### Preparation of (S)-10-amino-2-cyclopropyl-3,3-difluoro-7-methyl-1,2,3,4-tetrahydro-[1,4]oxazepino[2,3-c]quinolin-6(7H)-one

To a stirred solution of (S)-2-cyclopropyl-3,3-difluoro-7-methyl-10-nitro-1,2,3,4-tetrahydro-[1,4]oxazepino[2,3-c]quinolin-6(7H)-one (0.60 g, 1.7 mmol, prepared using published methods, e.g., International Application No. WO2019197842) in ethanol (28 mL) at 0° C. was added Zn powder (1.30 g, 19.95 mmol) and acetic acid (1.14 g, 19.95 mmol). The reaction mixture was stirred at room temperature for 3 h. The reaction mixture was filtered through a celite bed, concentrated, and purified by flash chromatography (230-400 mesh silica gel and 25%-27% of ethyl acetate in petroleum ether) to afford (S)-10-amino-2-cyclopropyl-3,3-difluoro-7-methyl-1,2,3,4-tetrahydro-[1,4]oxazepino[2,3-c]quinolin-6(7H)-one as a pale yellow semi solid (0.470 g).

LC-MS (ESI): m/z=322.36 [M+H]⁺.

### Preparation of (S)-2-chloro-4-((2-cyclopropyl-3,3-difluoro-7-methyl-6-oxo-1,2,3,4,6,7-hexahydro-[1,4]oxazepino[2,3-c]quinolin-10-yl)amino)pyrimidine-5-carbonitrile

To a stirred solution of (S)-10-amino-2-cyclopropyl-3,3-difluoro-7-methyl-1,2,3,4-tetrahydro-[1,4]oxazepino[2,3-c]quinolin-6(7H)-one (0.20 g, 0.62 mmol) in DMSO (2.0 mL) was added 2,4-dichloropyrimidine-5-carbonitrile (0.06 g, 0.37 mmol) and DIPEA (0.34 mL, 1.86 mmol). The reaction mixture was heated to 100° C. for 3 h. The reaction mixture was concentrated under reduced pressure and purified by Prep-HPLC to afford (S)-2-chloro-4-((2-cyclopropyl-3,3-difluoro-7-methyl-6-oxo-1,2,3,4,6,7-hexahydro-[1,4]oxazepino[2,3-c]quinolin-10-yl)amino)pyrimidine-5-carbonitrile as an off white solid. (0.12 g).

LC-MS (ESI): m/z=459.44 [M+H]⁺

### Preparation of 4-(((S)-2-cyclopropyl-3,3-difluoro-7-methyl-6-oxo-1,2,3,4,6,7-hexahydro-[1,4]oxazepino[2,3-c]quinolin-10-yl)amino)-2-(4-((1-(3-(2,6-dioxopiperidin-3-yl)-1-methyl-1H-indazol-7-yl)piperidin-4-yl)methyl)piperazin-1-yl)pyrimidine-5-carbonitrile

To a stirred solution of (S)-2-chloro-4-((2-cyclopropyl-3,3-difluoro-7-methyl-6-oxo-1,2,3,4,6,7-hexahydro-[1,4]oxazepino[2,3-c]quinolin-10-yl)amino)pyrimidine-5-carbonitrile (0.12 g, 0.261 mmol) in DMSO (2.0 mL) was added 3-(1-methyl-7-(4-(piperazin-1-ylmethyl)piperidin-1-yl)-1H-indazol-3-yl)piperidine-2,6-dione (0.06 g, 0.157 mmol) and N,N-diisopropylethylamine (0.10 mL, 0.786 mmol). The reaction mixture was heated at 100° C. for 3 h. The reaction mixture was concentrated under reduced pressure and purified by Prep-HPLC to afford 4-(((S)-2-cyclopropyl-3,3-difluoro-7-methyl-6-oxo-1,2,3,4,6,7-hexahydro-[1,4]oxazepino[2,3-c]quinolin-10-yl)amino)-2-(4-((1-(3-(2,6-dioxopiperidin-3-yl)-1-methyl-1H-indazol-7-yl)piperidin-4-yl)methyl)piperazin-1-yl)pyrimidine-5-carbonitrile (0.063 g) as an off white solid.

¹H NMR (400 MHz, DMSO-d₆): δ 10.88 (s, 1H), 9.69 (s, 1H), 9.42 (br s, 1H), 8.54 (br s, 1H), 8.18 (s, 1H), 7.62 (d, J=8.0 Hz, 1H), 7.38 (d, J=1.6 Hz, 1H), 7.05-7.01 (m, 2H), 6.26 (br s, 1H), 4.75 (br s, 1H) 4.49-4.34 (m, 4H), 4.24 (s, 3H), 3.61-3.33 (m, 3H), 3.03-2.70 (m, 9H), 2.82-2.58 (m, 6H), 2.32-2.29 (m, 1H), 2.18-2.14 (m, 1H), 2.15-1.86 (m, 3H), 1.51 (d, J=4.0 Hz, 2H), 1.52 (s, 1H), 0.71-0.70 (m, 1H), 0.53 (s, 2H), 0.33 (bs, 1H).

LC-MS (ESI): m/z=847.61 [M+H]⁺

### Example 82

### 3-(6-((4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)methyl)-1-methyl-1H-indazol-3-yl)piperidine-2,6-dione (Compound 439a)

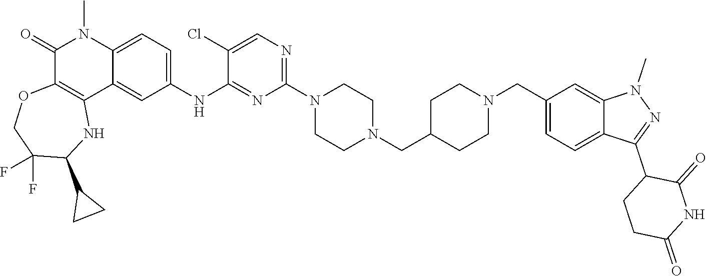 [SMILES: Cn1nc(C2CCC(=O)NC2=O)c2ccc(CN3CCC(CN4CCN(c5ncc(Cl)c(Nc6ccc7c(c6)c6c(c(=O)n7C)OCC(F)(F)[C@H](C7CC7)N6)n5)CC4)CC3)cc21]

### Preparation of 3-(2,6-bis(benzyloxy)pyridin-3-yl)-1-methyl-1H-indazole-6-carbaldehyde

To a solution of 3-(2,6-bis(benzyloxy)pyridin-3-yl)-6-bromo-1-methyl-1H-indazole (1.50 g, 3.0 mmol) in THF (15 mL) at −78° C. was added 4-methylmorpholine (0.26 mL, 2.40 mmol), n-BuLi (2.5M) (1.38 mL, 3.45 mmol) dropwise, and DMF (0.46 mL, 6.01 mmol). The reaction mixture was stirred at −78° C. for 5-10 min. The reaction mixture was quenched with aqueous ammonium chloride solution (1 M, 100 mL). The reaction mixture was warmed to room temperature and extracted with tert-butyl methyl ether (3×100 mL). The combined organic layers were washed with brine (100 mL), dried over anhydrous Na₂SO₄, filtered, and concentrated under and purified by column chromatography (SiO₂; 100-200 mesh, 40% EtOAc in petroleum ether) to afford 3-(2,6-bis(benzyloxy)pyridin-3-yl)-1-methyl-1H-indazole-6-carbaldehyde (0.9 g) as a pale-yellow liquid.

LC-MS (ESI): m/z=450.24 [M+H]⁺

### Preparation of tert-butyl 4-((1-((3-(2,6-bis(benzyloxy)pyridin-3-yl)-1-methyl-1H-indazol-6-yl) methyl) piperidin-4-yl) methyl) piperazine-1-carboxylate

To a solution of 3-(2,6-bis(benzyloxy)pyridin-3-yl)-1-methyl-1H-indazole-6-carbaldehyde (0.9 g, 2.0 mmol) and tert-butyl 4-(piperidin-4-ylmethyl) piperazine-1-carboxylate (0.56 g 2.0 mmol) in THF (18.0 mL) was added titanium (IV) isopropoxide (0.9 mL). The reaction mixture was stirred at room temperature for 4 h. Sodium cyanoborohydride (0.25 g, 4.0 mmol) was then added at 0° C., and the resulting mixture was stirred at room temperature for 8 h. The reaction mixture was quenched with water (100 mL) and extracted with DCM (2×100 mL). The combined organic layer was washed with brine (100 mL), dried over anhydrous Na₂SO₄, filtered, dried under vacuum, and purified by flash column chromatography (SiO₂, 230-400 mesh; 80% EtOAc in petroleum ether) to afford tert-butyl 4-((1-((3-(2,6-bis(benzyloxy)pyridin-3-yl)-1-methyl-1H-indazol-6-yl) methyl) piperidin-4-yl) methyl) piperazine-1-carboxylate (0.6 g) as an off white solid.

LC-MS (ESI): m/z=717.75 [M+H]⁺

### Preparation of tert-butyl 4-((1-((3-(2,6-dioxopiperidin-3-yl)-1-methyl-1H-indazol-6-yl) methyl) piperidin-4-yl) methyl) piperazine-1-carboxylate

To a solution of tert-butyl 4-((1-((3-(2,6-bis(benzyloxy)pyridin-3-yl)-1-methyl-1H-indazol-6-yl) methyl) piperidin-4-yl) methyl) piperazine-1-carboxylate (0.3 g, 0.41 mmol) in THF (15.0 mL) was added catalytic amount of acetic acid (0.01 mL), DMF (0.01 mL), and Pd(OH)₂ (0.58 g, 4.18 mmol). The reaction mixture was stirred under H₂ (35 psi) at room temperature for 8 h. The reaction mixture was quenched with ice-cold water (50 mL) and extracted into ethyl acetate (3×50 mL). The combined organic layers were washed with brine (50 mL), dried over anhydrous Na₂SO₄, filtered, dried under vacuum, and purified by flash column chromatography (SiO₂, 230-400 mesh; 100% EtOAc) to afford tert-butyl 4-((1-((3-(2,6-dioxopiperidin-3-yl)-1-methyl-1H-indazol-6-yl) methyl) piperidin-4-yl) methyl) piperazine-1-carboxylate (1.0 g) as a pale brown semi-solid.

LC-MS (ESI): m/z=539.65 [M+H]⁺

### Preparation of 3-(1-methyl-6-((4-(piperazin-1-ylmethyl) piperidin-1-yl) methyl)-1H-indazol-3-yl) piperidine-2,6-dione

To a solution of tert-butyl 4-((1-((3-(2,6-dioxopiperidin-3-yl)-1-methyl-1H-indazol-6-yl) methyl) piperidin-4-yl) methyl) piperazine-1-carboxylate (0.1 g, 0.18 mmol) in DCM (5.0 mL) at 0° C. was added TFA (1 mL). The reaction mixture was stirred at rt for 2 h. The reaction mixture was concentrated under reduced pressure and triturated with n-pentane (10 ml) to afford 3-(1-methyl-6-((4-(piperazin-1-ylmethyl) piperidin-1-yl) methyl)-1H-indazol-3-yl) piperidine-2,6-dione (0.06 g) as an off white solid.

LC-MS (ESI): m/z=439.73 [M+H]⁺.

### Preparation of 3-(6-((4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)methyl)-1-methyl-1H-indazol-3-yl)piperidine-2,6-dione

To a solution of 3-(1-methyl-6-((4-(piperazin-1-ylmethyl) piperidin-1-yl) methyl)-1H-indazol-3-yl) piperidine-2,6-dione (0.06 g, 0.13 mmol) in DMSO (0.6 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.03 g, 0.07 mmol) and DIPEA (0.07 mL). The reaction mixture was stirred at 100° C. for 3 h. The reaction mixture was concentrated under reduced pressure and purified by Prep-HPLC to afford 3-(6-((4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)methyl)-1-methyl-1H-indazol-3-yl)piperidine-2,6-dione (0.024 g) as an off white solid.

LC-MS (ESI): m/z=870.52 [M+H]⁺

¹HNMR: 400 MHz, DMSO-d6: δ 10.80 (s, 1H), 8.84 (s, 1H), 8.22 (d, J=2.00 Hz, 2H), 7.69 (dd, J=2.00, 9.00 Hz, 1H), 7.63 (d, J=8.40 Hz, 1H), 7.42-7.44 (m, 2H), 7.08 (d, J=9.20 Hz, 1H), 6.20 (s, 1H), 4.32-4.34 (m, 3H), 3.97 (s, 3H), 3.56 (s, 7H), 3.23-3.21 (m, 1H), 2.83-2.80 (m, 2H), 2.67-2.61 (m, 2H), 2.39-2.30 (m, 5H), 2.28-2.11 (m, 4H), 2.10-1.91 (m, 2H), 1.69-1.61 (m, 2H), 1.53-1.47 (m, 1H), 1.35-1.29 (m, 1H), 1.28-1.22 (m, 1H), 1.19-1.11 (m, 2H), 0.72-0.69 (m, 1H), 0.55-0.48 (m, 2H), 0.41-0.35 (m, 1H)

### Example 83: 3-(7-(4-((9-(5-chloro-4-(((S)-2-cyclopropyl-3,3-difluoro-7-methyl-6-oxo-1,2,3,4,6,7-hexahydro [1,4]oxazepino[2,3-c]quinolin-10-yl)amino)pyrimidin-2-yl)-3,9-diazaspiro[5.5]undecan-3-yl)methyl)piperidin-1-yl)-1-methyl-1H-indazol-3-yl)piperidine-2,6-dione (Compound 440a)

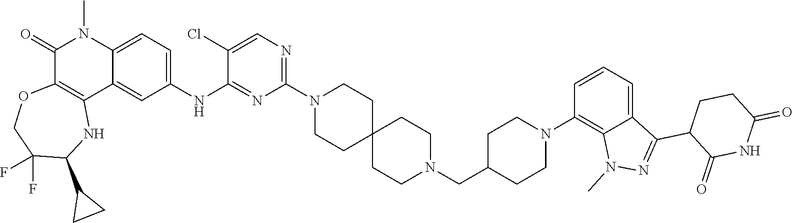 [SMILES: Cn1nc(C2CCC(=O)NC2=O)c2cccc(N3CCC(CN4CCC5(CC4)CCN(c4ncc(Cl)c(Nc6ccc7c(c6)c6c(c(=O)n7C)OCC(F)(F)[C@H](C7CC7)N6)n4)CC5)CC3)c21]

### Preparation of (1-(3-(2,6-bis(benzyloxy)pyridin-3-yl)-1-methyl-1H-indazol-7-yl)piperidin-4-yl)methanol

To a stirred solution of 3-(2,6-bis(benzyloxy)pyridin-3-yl)-7-bromo-1-methyl-1H-indazole (2.0 g, 4.0 mmol) in 1,4-dioxane (40 mL) was added 2-(piperidin-4-yl)ethan-1-ol (0.69 g, 6.00 mmol) and cesium carbonate (3.91 g, 12.0 mmol). The reaction mixture was purged with argon for 15 minutes, followed by addition of Pd PEPPSI iHept Cl (0.19 g, 0.20 mmol). The resulting mixture was then stirred and heated at 100° C. for 5 h. The reaction mixture was quenched with ice-cold water (30 mL) and extracted with ethyl acetate (3×150 mL). The combined organic layers were washed with brine (30 mL), dried over anhydrous Na₂SO₄, filtered, dried under vacuum, and purified by column chromatography (SiO₂, 100-200 mesh, 55% EtOAc in petroleum ether) to afford (1-(3-(2,6-bis(benzyloxy)pyridin-3-yl)-1-methyl-1H-indazol-7-yl)piperidin-4-yl)methanol (0.80 g) as a yellow solid.

LC-MS (ESI): m/z=535.62 [M+1]⁺

### Preparation of (1-(3-(2,6-bis(benzyloxy)pyridin-3-yl)-1-methyl-1H-indazol-7-yl)piperidin-4-yl)methyl methanesulfonate

A solution of (1-(3-(2,6-bis(benzyloxy)pyridin-3-yl)-1-methyl-1H-indazol-7-yl)piperidin-4-yl)methanol (0.80 g, 1.49 mmol)) in DCM (24 mL) was cooled to 0° C. TEA (0.61 mL, 4.49 mmol) and MsCl (0.30 mL, 4.49 mmol) were added. The resulting mixture was stirred for 2 h at room temperature. The reaction mixture was quenched with ice-cold water (30 mL) and extracted with ethyl acetate (3×150 mL). The combined organic layers were washed with brine (30 mL), dried over anhydrous Na₂SO₄, filtered, concentrated under vacuum, and washed with n-pentane to afford (1-(3-(2,6-bis(benzyloxy)pyridin-3-yl)-1-methyl-1H-indazol-7-yl)piperidin-4-yl)methyl methanesulfonate (0.8 g) as an orange semi-solid.

LC-MS (ESI): m/z=613.58 [M+H]⁺.

### Preparation of tert-butyl 9-((1-(3-(2,6-bis(benzyloxy)pyridin-3-yl)-1-methyl-1H-indazol-7-yl)piperidin-4-yl)methyl)-3,9-diazaspiro[5.5]undecane-3-carboxylate

To a solution of (1-(3-(2,6-bis(benzyloxy)pyridin-3-yl)-1-methyl-1H-indazol-7-yl)piperidin-4-yl)methyl methanesulfonate (0.8 g, 1.30 mmol) and tert-butyl 3,9-diazaspiro[5.5]undecane-3-carboxylate (1.32 g, 5.22 mmol) in DMSO (16 mL) was added N, N-diisopropylethylamine (2.27 mL, 13.00 mmol) and stirred at 100° C. for 16 h. The reaction mixture was diluted with ice water (50 mL) and extracted with 5% MeOH+DCM (3×70 mL). The combined organic layers were washed with brine, dried over anhydrous Na₂SO₄, filtered, concentrated under vacuum, and purified by column chromatography (52% EtOAc in petroleum ether) to afford tert-butyl 9-((1-(3-(2,6-bis(benzyloxy)pyridin-3-yl)-1-methyl-1H-indazol-7-yl)piperidin-4-yl)methyl)-3,9-diazaspiro[5.5]undecane-3-carboxylate (0.5 g) as an off white solid.

LC-MS (ESI): m/z=771.55 [M+H]⁺

### Preparation of tert-butyl 9-((1-(3-(2,6-dioxopiperidin-3-yl)-1-methyl-1H-indazol-7-yl)piperidin-4-yl)methyl)-3,9-diazaspiro[5.5]undecane-3-carboxylate

A stirred solution of tert-butyl 9-((1-(3-(2,6-bis(benzyloxy)pyridin-3-yl)-1-methyl-1H-indazol-7-yl)piperidin-4-yl)methyl)-3,9-diazaspiro[5.5]undecane-3-carboxylate (0.5 g, 0.64 mmol) in THF (15 mL) was degassed with nitrogen. To the solution was then added 20% Pd(OH)₂ on carbon (0.5 g). The reaction mixture was stirred under H₂ (80 psi) at room temperature for 16 h. The reaction mixture was filtered through a celite bed, concentrated under reduced pressure, and washed with n-pentane to afford tert-butyl 9-((1-(3-(2,6-dioxopiperidin-3-yl)-1-methyl-1H-indazol-7-yl)piperidin-4-yl)methyl)-3,9-diazaspiro[5.5]undecane-3-carboxylate (0.4 g) as a green solid.

LC-MS (ESI): m/z=593.82 [M+H]⁺.

### Preparation of 3-(7-(4-((3,9-diazaspiro[5.5]undecan-3-yl)methyl)piperidin-1-yl)-1-methyl-1H-indazol-3-yl)piperidine-2,6-dione

A solution of tert-butyl 9-((1-(3-(2,6-dioxopiperidin-3-yl)-1-methyl-1H-indazol-7-yl)piperidin-4-yl)methyl)-3,9-diazaspiro[5.5]undecane-3-carboxylate (0.40 g, 0.67 mmol)) in DCM (4.0 mL) was cooled to 0° C. TFA (4.0 mL) was then added. The resulting mixture was stirred at room temperature for 2 h, concentrated under reduced pressure, and washed with diethyl ether to afford 3-(7-(4-((3,9-diazaspiro[5.5]undecan-3-yl)methyl)piperidin-1-yl)-1-methyl-1H-indazol-3-yl)piperidine-2,6-dione (0.25 g) as a brown solid.

LC-MS (ESI): m/z=493.69 [M+H]⁺.

### Preparation of 3-(7-(4-((9-(5-chloro-4-(((S)-2-cyclopropyl-3,3-difluoro-7-methyl-6-oxo-1,2,3,4,6,7-hexahydro [1,4]oxazepino[2,3-c]quinolin-10-yl)amino)pyrimidin-2-yl)-3,9-diazaspiro[5.5]undecan-3-yl)methyl)piperidin-1-yl)-1-methyl-1H-indazol-3-yl)piperidine-2,6-dione

To a solution of 3-(7-(4-((3,9-diazaspiro[5.5]undecan-3-yl)methyl)piperidin-1-yl)-1-methyl-1H-indazol-3-yl)piperidine-2,6-dione (0.10 g, 0.20 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.04 g, 0.10 mmol) in DMSO (3 mL) was added N,N-diisopropylethylamine (0.27 mL, 1.60 mmol). The reaction mixture was stirred at 100° C. for 16 h.

The reaction mixture was concentrated under reduced pressure and purified by Prep-HPLC to afford 3-(7-(4-((9-(5-chloro-4-(((S)-2-cyclopropyl-3,3-difluoro-7-methyl-6-oxo-1,2,3,4,6,7-hexahydro [1,4]oxazepino[2,3-c]quinolin-10-yl)amino)pyrimidin-2-yl)-3,9-diazaspiro[5.5]undecan-3-yl)methyl)piperidin-1-yl)-1-methyl-1H-indazol-3-yl)piperidine-2,6-dione (0.026 g) as an off white solid.

LC-MS (ESI): m/z=924.70 [M+H]⁺

¹H NMR (400 MHz, DMSO-d6): δ 10.90 (s, 1H), 8.85 (s, 1H), 8.78 (s, 1H), 8.20 (d, J=2.00 Hz, 1H), 8.02 (s, 1H), 7.73 (q, J=2.00 Hz, 1H), 7.44 (d, J=9.20 Hz, 1H), 7.35 (q, J=2.40 Hz, 1H), 0.00 (d, J=6.40 Hz, 2H), 6.20 (s, 1H), 4.36-4.38 (m, 4H), 4.23 (s, 3H), 3.65 (s, 7H), 3.35 (s, 3H), 2.67 (m, 3H), 2.50 (d, J=1.60 Hz, 4H), 2.33-2.32 (m, 2H), 1.85 (t, J=10.80 Hz, 2H), 1.66 (s, 2H), 1.45 (s, 4H), 1.36 (s, 7H), 1.24 (s, 1H), 0.73-0.72 (m, 1H), 0.51 (t, J=5.60 Hz, 2H), 0.35 (s, 1H).

### Example 84: (S)-1-(7-(4-((4-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)dihydropyrimidine-2,4(1H, 3H)-dione (Compound 170a)

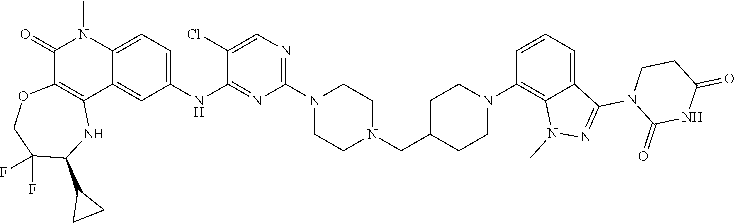 [SMILES: Cn1nc(N2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)CC4)CC3)c21]

### Preparation of 7-bromo-1-methyl-1H-indazol-3-amine

A solution of 3-bromo-2-fluorobenzonitrile (25.00 g, 124.99 mmol) in ethanol (400 mL) was degassed with nitrogen for 10 minutes, followed by the addition of methyl hydrazine (85% in aqueous) (11.52 g, 249.99 mmol) at 0° C. The resulting mixture was then stirred at 120° C. for 18 h and concentrated under vacuum to obtain a residue, which was then purified by column chromatography (SiO₂; 40% EtOAc in petroleum ether) to afford 7-bromo-1-methyl-1H-indazol-3-amine (18.0 g) as a brown semi-solid.

LC-MS (ESI): m/z=226.09 [M+H]⁺

### Preparation of 3-((7-bromo-1-methyl-1H-indazol-3-yl) amino) propanoic acid

To a stirred solution of 7-bromo-1-methyl-1H-indazol-3-amine (17.00 g, 75.20 mmol) in HCl (170 mL, 2N), acrylic acid (8.90 mL, 99.52 mmol) and TBAB (4.84 g, 15.04 mmol) was added and heated to 100° C. for 18 h. The reaction mixture was then quenched with ice cold water (200 mL) and extracted with 10% MeOH in ethyl acetate (5×100 mL). The separated organic layers were combined, washed with brine solution (100 mL), dried over anhydrous Na₂SO₄, filtered, and concentrated under vacuum to afford 3-((7-bromo-1-methyl-1H-indazol-3-yl) amino) propanoic acid (15.6 g) as pale brown semi-solid which was used in the next step without purification.

LC-MS (ESI): m/z=298.03 [M+H]⁺.

### Preparation of 1-(7-bromo-1-methyl-1H-indazol-3-yl) dihydropyrimidine-2,4(1H, 3H)-dione

To a stirred solution of 3-((7-bromo-1-methyl-1H-indazol-3-yl) amino) propanoic acid (15.00 g, 50.31 mmol) in acetic acid (150 mL) was added sodium cyanate (13.08 g, 201.25 mmol). The reaction mixture was heated to 75° C. for 18 h, followed by the addition of 4N HCl (180 mL). The resulting mixture was then stirred for an additional 5 h. The reaction mixture was quenched with ice cold water (500 mL). The precipitated solid was filtered, dried under vacuum, and washed with n-pentane to afford 1-(7-bromo-1-methyl-1H-indazol-3-yl) dihydropyrimidine-2,4(1H, 3H)-dione (3.91 g) as a brown solid.

LC-MS (ESI): m/z=324.33 [M+H]⁺.

### Preparation of tert-butyl 4-((1-(3-(2,4-dioxotetrahydropyrimidin-1(2H)-yl)-1-methyl-1H-indazol-7-yl) piperidin-4-yl) methyl) piperazine-1-carboxylate

A stirred solution of 1-(7-bromo-1-methyl-1H-indazol-3-yl) dihydropyrimidine-2,4(1H, 3H)-dione (3.10 g, 9.59 mmol), tert-butyl 4-(piperidin-4-ylmethyl) piperazine-1-carboxylate (9.52 g, 33.58 mmol) and Cs₂CO₃ (15.63 g, 47.96 mmol) in dioxane (31 mL) and DMF (15.50 mL) was degassed with argon for 10 minutes. Pd-PEPPSI-iHeptCl (0.47 g, 0.48 mmol) was then added. The resulting mixture was heated to 120° C. for 2 h under microwave irradiation. The reaction mixture was filtered through a celite bed, concentrated under vacuum, and purified by column chromatography (SiO₂, 100-200 mesh; 80-90% EtOAc in petroleum ether) to afford tert-butyl 4-((1-(3-(2,4-dioxotetrahydropyrimidin-1(2H)-yl)-1-methyl-1H-indazol-7-yl) piperidin-4-yl) methyl) piperazine-1-carboxylate (1.22 g) as a pale brown semi-solid.

LC-MS (ESI): m/z=526.67 (M+H)+.

### Preparation of 1-(1-methyl-7-(4-(piperazin-1-ylmethyl) piperidin-1-yl)-1H-indazol-3-yl) dihydropyrimidine-2,4(1H, 3H)-dione

To a solution of tert-butyl 4-((1-(3-(2,4-dioxotetrahydropyrimidin-1(2H)-yl)-1-methyl-1H-indazol-7-yl) piperidin-4-yl) methyl) piperazine-1-carboxylate (1.20 g, 2.28 mmol) in DCM (12.0 mL) was added TFA (6.0 mL) at 0° C. The reaction mixture was stirred at room temperature for 1 h. The reaction mixture was concentrated under reduced pressure and triturated with n-pentane to afford 1-(1-methyl-7-(4-(piperazin-1-ylmethyl) piperidin-1-yl)-1H-indazol-3-yl) dihydropyrimidine-2,4(1H, 3H)-dione (0.95 g) as a pale brown semi solid.

LC-MS (ESI): m/z=426.49 [M+H]⁺.

### Preparation of (S)-1-(7-(4-((4-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)dihydropyrimidine-2,4(1H, 3H)-dione

To a stirred solution of 1-(1-methyl-7-(4-(piperazin-1-ylmethyl) piperidin-1-yl)-1H-indazol-3-yl) dihydropyrimidine-2,4(1H, 3H)-dione (0.32 g, 0.752 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.11 g, 0.225 mmol) in DMSO (3.2 mL) was added DIPEA (3.2 mL). The mixture was heated to 100° C. for 18 h. The reaction mixture was concentrated under reduced pressure and purified by prep-HPLC to afford (S)-1-(7-(4-((4-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-1-methyl-1H-indazol-3-yl)dihydropyrimidine-2,4(1H, 3H)-dione (0.10 g) as an off white solid.

LC-MS (ESI): m/z=857.57 [M+H]⁺

¹H NMR (400 MHz, DMSO-d₆): δ 10.57 (s, 1H), 8.85 (s, 1H), 8.23 (d, J=2.00 Hz, 1H), 8.05 (s, 1H), 7.70 (dd, J=2.00, 9.20 Hz, 1H), 7.44 (d, J=8.80 Hz, 1H), 7.28-7.27 (m, 1H), 7.01-7.00 (m, 2H), 6.22 (s, 1H), 4.41-4.38 (m, 2H), 4.23 (s, 3H), 3.88-3.86 (m, 2H), 3.59 (s, 4H), 3.56 (s, 3H), 3.23-3.21 (m, 3H), 2.75-2.74 (m, 2H), 2.67-2.67 (m, 2H), 2.37-2.35 (m, 4H), 2.33-2.32 (m, 2H), 1.88-1.85 (m, 2H), 1.34-1.31 (m, 3H), 0.72-0.70 (m, 2H), 0.52-0.51 (m, 2H), 0.36-0.35 (m, 1H).

### Example 85: 3-(6-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[3.5]nonan-2-yl)methyl)piperidin-1-yl)-1-methyl-1H-indazol-3-yl)piperidine-2,6-dione (Compound 445a)

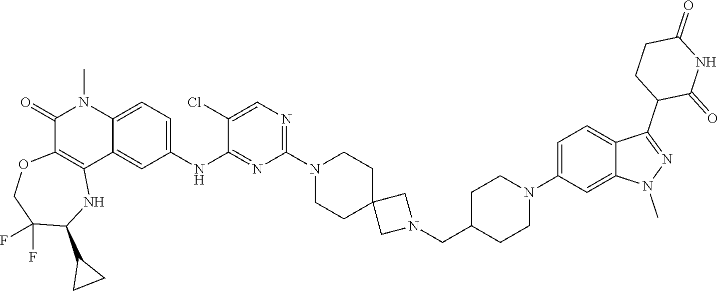 [SMILES: Cn1nc(C2CCC(=O)NC2=O)c2ccc(N3CCC(CN4CC5(CCN(c6ncc(Cl)c(Nc7ccc8c(c7)c7c(c(=O)n8C)OCC(F)(F)[C@H](C8CC8)N7)n6)CC5)C4)CC3)cc21]

### Preparation of (1-(3-(2,6-bis(benzyloxy)pyridin-3-yl)-1-methyl-1H-indazol-6-yl) piperidin-4-yl) methanol

To a stirred solution of piperidin-4-ylmethanol (0.6 g, 5.21 mmol) and 3-(2,6-bis(benzyloxy)pyridin-3-yl)-6-bromo-1-methyl-1H-indazole (1.04 g, 2.08 mmol) in 1,4-dioxane (20 mL) was added cesium carbonate (5.0 g, 15.6 mmol). The reaction mixture was degassed with nitrogen for 10 min. Afterwards, Pd-PEPPSI-iHept-Cl (0.25 g, 0.26 mmol) was added, and the resultant reaction mixture was stirred at 100° C. for 3 h. After completion, the reaction mixture was filtered through a celite bed, washed with ethyl acetate (100 mL), concentrated under reduced pressure, and purified by flash column chromatography (SiO₂, 60-120 mesh, 25% ethyl acetate in petroleum ether) to afford (1-(3-(2,6-bis(benzyloxy)pyridin-3-yl)-1-methyl-1H-indazol-6-yl) piperidin-4-yl) methanol (0.8 g).

LCMS (ESI): m/z=535.66 [M+H]⁺

### Preparation of (1-(3-(2,6-bis(benzyloxy)pyridin-3-yl)-1-methyl-1H-indazol-6-yl)piperidin-4-yl)methyl methanesulfonate

To a solution of (1-(3-(2,6-bis(benzyloxy)pyridin-3-yl)-1-methyl-1H-indazol-6-yl) piperidin-4-yl) methanol (0.8 g, 1.49 mmol) and triethylamine (0.64 mL, 4.49 mmol) in DCM (16 mL) was added methanesulfonyl chloride (0.13 mL, 1.79 mmol) at 0° C. The reaction mixture was stirred at rt for 2 h. The reaction mixture was filtered through a celite bed, concentrated under reduced pressure, washed with n-pentane (30 ml), and dried under vacuum to afford (1-(3-(2,6-bis(benzyloxy)pyridin-3-yl)-1-methyl-1H-indazol-6-yl)piperidin-4-yl)methyl methanesulfonate (0.7 g), which was used in next step without any purification.

LC-MS (ESI): m/z=613.66 [M+H]⁺

### Preparation of tert-butyl 2-((1-(3-(2,6-bis(benzyloxy)pyridin-3-yl)-1-methyl-1H-indazol-6-yl) piperidin-4-yl) methyl)-2,7-diazaspiro [3.5]nonane-7-carboxylate

To a solution of (1-(3-(2,6-bis(benzyloxy)pyridin-3-yl)-1-methyl-1H-indazol-6-yl)piperidin-4-yl)methyl methanesulfonate (0.7 g, 1.14 mmol) and tert-butyl 2,7-diazaspiro [3.5]nonane-7-carboxylate (0.31 g, 1.37 mmol) in acetonitrile (14 mL) was added sodium carbonate (0.24 g, 2.28 mmol) and potassium iodide (0.03 g, 0.19 mmol). The reaction mixture was stirred at 100° C. for 16 h. After completion of the reaction, the residue was diluted with water (50 mL) and extracted with EtOAc (3×50 mL). The combined organic layers were dried over anhydrous sodium sulfate, filtered, concentrated under reduced pressure, and purified by flash column chromatography (SiO₂, 60-120 mesh, 2% methanol in DCM) to afford tert-butyl 2-((1-(3-(2,6-bis(benzyloxy)pyridin-3-yl)-1-methyl-1H-indazol-6-yl) piperidin-4-yl) methyl)-2,7-diazaspiro [3.5]nonane-7-carboxylate (0.6 g) as a brown solid.

LC-MS (ESI): m/z=743.79 [M+H]⁺

### Preparation of tert-butyl 2-((1-(3-(2,6-dioxopiperidin-3-yl)-1-methyl-1H-indazol-6-yl) piperidin-4-yl) methyl)-2,7-diazaspiro [3.5]nonane-7-carboxylate

A stirred solution of tert-butyl 2-((1-(3-(2,6-bis(benzyloxy)pyridin-3-yl)-1-methyl-1H-indazol-6-yl) piperidin-4-yl) methyl)-2,7-diazaspiro [3.5]nonane-7-carboxylate (0.6 g, 0.8 mmol) in a mixture of THF (18 mL) and AcOH (0.06 mL) was degassed with nitrogen for 5 minutes.

Afterwards, 20% Pd(OH)₂ on carbon (0.30 g, 50% w/w) was added, and the resulting mixture was stirred under a hydrogen atmosphere (15 psi) at room temperature for 16 h. After completion, the reaction mixture was diluted with ethyl acetate (35 mL), filtered through a celite bed, and washed with ethyl acetate (100 mL). The filtrate was collected, concentrated under reduced pressure and purified by trituration with diethyl ether (50 ml) to afford tert-butyl 2-((1-(3-(2,6-dioxopiperidin-3-yl)-1-methyl-1H-indazol-6-yl) piperidin-4-yl) methyl)-2,7-diazaspiro [3.5]nonane-7-carboxylate (0.4 g).

LC-MS (ESI): m/z=565.42 [M+H]⁺

### Preparation of 3-(6-(4-((2,7-diazaspiro [3.5]nonan-2-yl) methyl) piperidin-1-yl)-1-methyl-1H-indazol-3-yl) piperidine-2,6-dione

To a stirred solution of tert-butyl 2-((1-(3-(2,6-dioxopiperidin-3-yl)-1-methyl-1H-indazol-6-yl) piperidin-4-yl) methyl)-2,7-diazaspiro [3.5]nonane-7-carboxylate (0.4 g, 0.7 mmol) in dichloromethane (4.0 mL) was added trifluoroacetic acid (2.0 mL) at 0° C. under a N₂ atmosphere.

The resulting mixture was stirred at room temperature for 3 h. After completion, the reaction mixture was concentrated under reduced pressure and purified by trituration with diethyl ether (10 mL) to afford 3-(6-(4-((2,7-diazaspiro [3.5]nonan-2-yl) methyl) piperidin-1-yl)-1-methyl-1H-indazol-3-yl) piperidine-2,6-dione (0.35 g).

LC-MS (ESI): m/z=465.68 [M+H]⁺

### Preparation of 3-(6-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[3.5]nonan-2-yl)methyl)piperidin-1-yl)-1-methyl-1H-indazol-3-yl)piperidine-2,6-dione

To a solution of 3-(6-(4-((2,7-diazaspiro [3.5]nonan-2-yl) methyl) piperidin-1-yl)-1-methyl-1H-indazol-3-yl) piperidine-2,6-dione (0.15 g, 0.39 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.06 g, 0.15 mmol) in DMSO (3.0 mL) was added DIPEA (0.34 mL, 1.9 mmol). The reaction mixture was stirred at 100° C. for 3 h. The reaction mixture was concentrated under reduced pressure and purified by prep-HPLC to afford 3-(6-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[3.5]nonan-2-yl)methyl)piperidin-1-yl)-1-methyl-1H-indazol-3-yl)piperidine-2,6-dione (0.045 g) as an off white solid.

LC-MS (ESI): m/z=896.53 [M+H]⁺.

¹H NMR (400 MHz, DMSO-d₆): δ 10.82 (s, 1H), 8.81 (s, 1H), 8.17 (s, 1H), 8.02 (s, 1H), 7.71 (dd, J=1.60, 9.20 Hz, 1H), 7.44 (dd, J=5.20, 8.60 Hz, 2H), 6.53 (d, J=8.80 Hz, 1H), 6.32 (s, 1H), 6.21 (s, 1H), 4.37-4.35 (m, 2H), 4.26-4.23 (m, 1H), 3.83 (s, 3H), 3.56-3.53 (m, 12H), 3.20 (s, 3H), 2.92-2.89 (m, 6H), 2.60-2.59 (m, 2H), 2.26-2.25 (m, 2H), 2.15-2.13 (m, 4H), 1.44-1.42 (m, 2H), 1.32 (br, 1H), 0.71-0.70 (m, 1H), 0.52-0.51 (m, 2H), 0.35-0.34 (m, 1H).

### Example 86: 3-(7-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[3.5]nonan-2-yl)methyl)piperidin-1-yl)-1-methyl-1H-indazol-3-yl)piperidine-2,6-dione (Compound 446a)

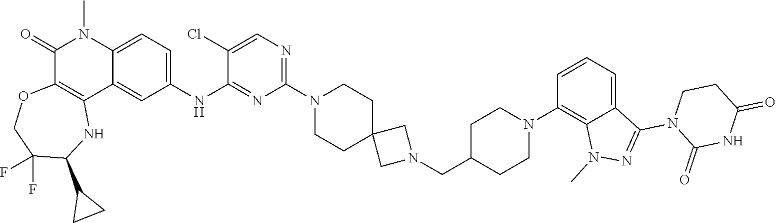 [SMILES: Cn1nc(N2CCC(=O)NC2=O)c2cccc(N3CCC(CN4CC5(CCN(c6ncc(Cl)c(Nc7ccc8c(c7)c7c(c(=O)n8C)OCC(F)(F)[C@H](C8CC8)N7)n6)CC5)C4)CC3)c21]

### Preparation of (1-(3-(2,6-bis(benzyloxy)pyridin-3-yl)-1-methyl-1H-indazol-7-yl) piperidin-4-yl) methanol

A solution of 3-(2,6-bis(benzyloxy) pyridin-3-yl)-7-bromo-1-methyl-1H-indazole (2.5 g, 4.99 mmol), piperidin-4-ylmethanol (1.72 g, 14.98 mmol), and cesium carbonate (5.69 g, 17.48 mmol) in 1,4-dioxane (50.0 mL) was degassed with argon for 10 minutes. Pd-PEPPSI-iHeptCl (0.24 g, 0.24 mmol) was then added. The resulting mixture was stirred and heated at 100° C. for 18 h. The reaction mixture was then diluted with DCM (300 mL) and filtered through a celite bed. The collected filtrate was concentrated under vacuum to obtain a residue which was purified by flash column chromatography (SiO₂, 230-400 mesh, 90% ethyl acetate in petroleum ether as eluent) to afford (1-(3-(2,6-bis(benzyloxy)pyridin-3-yl)-1-methyl-1H-indazol-7-yl) piperidin-4-yl) methanol (0.51 g) as a pale yellow solid.

LC-MS (ESI): m/z=535.60 [M+H]⁺.

### Preparation of (1-(3-(2,6-bis(benzyloxy)pyridin-3-yl)-1-methyl-1H-indazol-7-yl)piperidin-4-yl)methyl methanesulfonate

To a solution of (1-(3-(2,6-bis(benzyloxy)pyridin-3-yl)-1-methyl-1H-indazol-7-yl) piperidin-4-yl) methanol (0.20 g, 0.37 mmol) and triethylamine (0.09 g, 0.93 mmol) in DCM (2.0 mL) was added methanesulfonyl chloride (0.05 g, 0.44 mmol). The reaction mixture was stirred at room temperature for 2 h. The resulting mixture was poured into water (15 mL) and extracted with DCM (2×20 mL). The separated organic layers were combined, washed with brine (10 mL), dried over sodium sulfate, filtered, and concentrated under vacuum to obtain a residue which was triturated with n-pentane (5 mL), and dried under vacuum to afford (1-(3-(2,6-bis(benzyloxy)pyridin-3-yl)-1-methyl-1H-indazol-7-yl)piperidin-4-yl)methyl methanesulfonate (0.20 g) as a pale brown solid.

LC-MS (ESI): m/z=613.66 [M+H]⁺

### Preparation of tert-butyl 2-((1-(3-(2,6-bis(benzyloxy)pyridin-3-yl)-1-methyl-1H-indazol-7-yl) piperidin-4-yl) methyl)-2,7-diazaspiro [3.5]nonane-7-carboxylate

To a solution of (1-(3-(2,6-bis(benzyloxy)pyridin-3-yl)-1-methyl-1H-indazol-7-yl)piperidin-4-yl)methyl methanesulfonate (0.21 g, 0.34 mmol), tert-butyl 2,7-diazaspiro [3.5]nonane-7-carboxylate (0.11 g, 0.51 mmol), and sodium carbonate (0.14 g, 1.36 mmol) in MeCN (4.2 mL) was added potassium iodide (0.05 g, 0.03 mmol) and stirred at 90° C. for 16 h. The reaction mixture was quenched with water (15 mL) and extracted with EtOAc (2×30 mL). The separated organic layers were washed with brine (10 mL), dried over anhydrous Na₂SO₄, filtered, concentrated under reduced pressure, and purified by flash column chromatography (SiO₂, 100-200 mesh; 5% methanol in dichloromethane as eluent) to afford tert-butyl 2-((1-(3-(2,6-bis(benzyloxy)pyridin-3-yl)-1-methyl-1H-indazol-7-yl) piperidin-4-yl) methyl)-2,7-diazaspiro [3.5]nonane-7-carboxylate (0.20 g) as a yellow semi-solid.

LC-MS (ESI): m/z=743.83 [M+H]⁺

### Preparation of tert-butyl 2-((1-(3-(2,6-dioxopiperidin-3-yl)-1-methyl-1H-indazol-7-yl) piperidin-4-yl) methyl)-2,7-diazaspiro [3.5]nonane-7-carboxylate

To a solution of tert-butyl 2-((1-(3-(2,6-bis(benzyloxy)pyridin-3-yl)-1-methyl-1H-indazol-7-yl) piperidin-4-yl) methyl)-2,7-diazaspiro [3.5]nonane-7-carboxylate (0.200 g, 0.27 mmol) and AcOH (0.2 mL) in THF (6.0 mL) was added 20% Pd(OH)₂ on carbon (0.200 g, 100% w/w). The reaction mixture was stirred under hydrogen atmosphere (15 psi) at room temperature for 16 h. The reaction mixture was diluted with THF (30 mL), filtered through a celite bed, and washed with excess of THF:DCM (1:1, 150 mL). The collected filtrate was concentrated and dried under vacuum to afford tert-butyl 2-((1-(3-(2,6-dioxopiperidin-3-yl)-1-methyl-1H-indazol-7-yl) piperidin-4-yl) methyl)-2,7-diazaspiro [3.5]nonane-7-carboxylate (0.13 g) as a brown semi-solid which was used in the next step without further purification.

LC-MS (ESI): m/z=565.65 [M+H]⁺.

### Preparation of 3-(7-(4-((2,7-diazaspiro [3.5]nonan-2-yl) methyl) piperidin-1-yl)-1-methyl-1H-indazol-3-yl) piperidine-2,6-dione

To a solution of tert-butyl 2-((1-(3-(2,6-dioxopiperidin-3-yl)-1-methyl-1H-indazol-7-yl) piperidin-4-yl) methyl)-2,7-diazaspiro [3.5]nonane-7-carboxylate (0.13 g, 0.23 mmol) in DCM (2.6 mL) was added TFA (1.3 mL) at 0° C. The reaction mixture was stirred at room temperature for 3 h. The reaction mixture was concentrated under reduced pressure and co-distilled with DCM (2×10 mL) to afford 3-(7-(4-((2,7-diazaspiro [3.5]nonan-2-yl) methyl) piperidin-1-yl)-1-methyl-1H-indazol-3-yl) piperidine-2,6-dione (0.13 g) as a pale brown semi-solid.

LC-MS (ESI): m/z=465.55 [M+H]⁺.

### Preparation of 3-(7-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[3.5]nonan-2-yl)methyl)piperidin-1-yl)-1-methyl-1H-indazol-3-yl)piperidine-2,6-dione

To a stirred solution of 3-(7-(4-((2,7-diazaspiro [3.5]nonan-2-yl) methyl) piperidin-1-yl)-1-methyl-1H-indazol-3-yl) piperidine-2,6-dione (0.13 g, 0.28 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.07 g, 0.14 mmol) in DMSO (2.6 mL) was added DIPEA (1.3 mL). The mixture was heated at 120° C. for 4 h. The reaction mixture was concentrated under reduced pressure, and the resulting residue was purified by prep HPLC to afford 3-(7-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,7-diazaspiro[3.5]nonan-2-yl)methyl)piperidin-1-yl)-1-methyl-1H-indazol-3-yl)piperidine-2,6-dione (0.015 g) as an off white solid.

LC-MS (ESI): m/z=896.57 [M+H]⁺.

¹H NMR (400 MHz, DMSO-d₆): δ 10.87 (s, 1H), 8.81 (s, 1H), 8.18 (d, J=1.60 Hz, 1H), 8.02 (s, 1H), 7.72 (dd, J=2.00, 9.00 Hz, 1H), 7.44 (d, J=9.20 Hz, 1H), 7.35 (d, J=2.40 Hz, 1H), 7.00-6.99 (m, 2H), 6.21 (br s, 1H), 4.42-4.41 (m, 3H), 4.22 (s, 3H), 3.56-3.54 (m, 7H), 3.21-3.20 (m, 3H), 2.94 (s, 3H), 2.62-2.61 (m, 3H), 2.42-2.41 (m, 3H), 2.31-2.29 (m, 1H), 2.17-2.15 (m, 1H), 1.81 (d, J=11.60 Hz, 3H), 1.62 (br s, 3H), 1.36-1.34 (m, 3H), 1.24 (s, 1H), 0.72-0.68 (m, 2H), 0.52-0.51 (m, 2H), 0.35-0.34 (m, 1H).

### Example 87: 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)oxy)-1-methyl-1H-indazol-3-yl)piperidine-2,6-dione (Compound 447a)

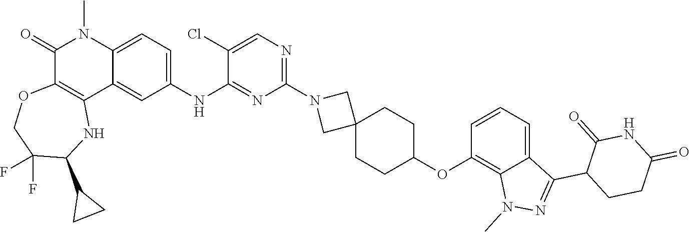 [SMILES: Cn1nc(C2CCC(=O)NC2=O)c2cccc(OC3CCC4(CC3)CN(c3ncc(Cl)c(Nc5ccc6c(c5)c5c(c(=O)n6C)OCC(F)(F)[C@H](C6CC6)N5)n3)C4)c21]

### Preparation of 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)oxy)-1-methyl-1H-indazol-3-yl)piperidine-2,6-dione

To a stirred solution of 3-(7-((2-azaspiro [3.5]nonan-7-yl) oxy)-1-methyl-1H-indazol-3-yl) piperidine-2,6-dione (0.20 g, 0.47 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.11 g, 0.23 mmol) in DMSO (4 mL) was added N,N-diisopropylethylamine (0.70 mL, 3.76 mmol).

The resulting mixture was heated at 100° C. for 2 h, cooled to ambient temperature, and then purified by preparative reverse-phase HPLC to afford 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)oxy)-1-methyl-1H-indazol-3-yl)piperidine-2,6-dione (35 mg) as an off white solid.

LC-MS (ESI): m/z=814.47 [M+H]⁺.

¹H NMR data (400 MHz, DMSO-d₆): δ 10.87 (s, 1H), 8.76 (s, 1H), 8.28 (s, 1H), 8.03 (s, 1H), 7.82 (d, J=9.20 Hz, 1H), 7.44 (d, J=9.20 Hz, 1H), 7.19 (d, J=8.00 Hz, 1H), 6.95-6.97 (m, 1H), 6.88 (d, J=7.20 Hz, 1H), 6.13 (s, 1H), 4.60 (br s, 1H), 4.41-4.42 (m, 2H), 4.29-4.30 (m, 1H), 4.18 (s, 3H), 3.71 (s, 4H), 3.57 (s, 3H), 2.61-2.63 (m, 2H), 2.16-2.18 (m, 1H), 1.89-1.91 (m, 4H), 1.65-1.69 (m, 4H), 1.31-1.33 (m, 2H), 0.69-0.67 (m, 2H), 0.51-0.52 (m, 2H), 0.36-0.37 (m, 1H).

### Example 88: 3-(7-((4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)methyl)-1-methyl-1H-indazol-3-yl)piperidine-2,6-dione (Compound 448a)

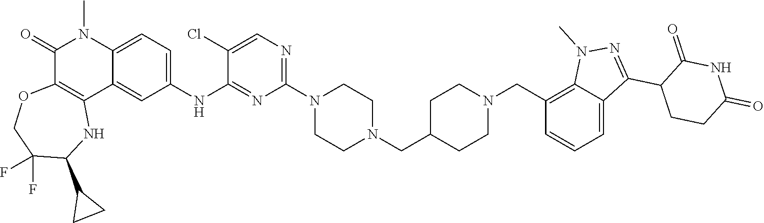 [SMILES: Cn1nc(C2CCC(=O)NC2=O)c2cccc(CN3CCC(CN4CCN(c5ncc(Cl)c(Nc6ccc7c(c6)c6c(c(=O)n7C)OCC(F)(F)[C@H](C7CC7)N6)n5)CC4)CC3)c21]

### Preparation of 3-(2,6-bis(benzyloxy)pyridin-3-yl)-1-methyl-1H-indazole-7-carbaldehyde

To a stirred solution of 3-(2,6-bis(benzyloxy)pyridin-3-yl)-6-bromo-1-methyl-1H-indazole (1.0 g, 2.0 mmol) in THF (10.0 mL) at −78° C. was added 4-methylmorpholine (0.17 mL, 1.60 mmol). ⁿBuLi (2.5M in hexane) (0.92 mL, 2.30 mmol) drop wise at −78° C. over a period of 10 min, followed by DMF (0.31 mL, 4.0 mmol). The resulting mixture was stirred at −78° C. for 5 min. The reaction mixture was then quenched with aqueous ammonium chloride solution (10 mL, 1 M), warmed to room temperature, and extracted with methyl tert-butyl ether (3×20 mL). The combined organic layers were washed with brine (10 mL), dried over Na₂SO₄, filtered, dried under vacuum, and purified by column chromatography (SiO₂; 40% EtOAc in petroleum ether) to afford 3-(2,6-bis(benzyloxy)pyridin-3-yl)-1-methyl-1H-indazole-7-carbaldehyde (0.4 g) as a pale-yellow liquid.

LC-MS (ESI): m/z=450.24 [M+H]⁺.

### Preparation of tert-butyl 4-((1-((3-(2,6-bis(benzyloxy)pyridin-3-yl)-1-methyl-1H-indazol-7-yl) methyl) piperidin-4-yl) methyl) piperazine-1-carboxylate

To a stirred solution of 3-(2,6-bis(benzyloxy)pyridin-3-yl)-1-methyl-1H-indazole-7-carbaldehyde (0.25 g, 0.55 mmol), tert-butyl 4-(piperidin-4-ylmethyl) piperazine-1-carboxylate (0.15 g 0.55 mmol) in THF (5.0 mL) was added titanium (IV) isopropoxide (0.25 mL) at room temperature. The reaction mixture was stirred at room temperature for 1 h. Afterwards, sodium cyanoborohydride (0.07 g, 1.11 mmol) was added at 0° C., and the resulting mixture was stirred at room temperature for 12 h. The reaction mixture was quenched with water (30.0 mL) and extracted with DCM (100.0 mL). The separated organic layers were combined, washed with brine (30.0 mL), dried over anhydrous Na₂SO₄, filtered, and dried under vacuum to get a residue which was purified by flash column chromatography (SiO₂, 230-400 mesh; 80% EtOAc in petroleum ether) to afford tert-butyl 4-((1-((3-(2,6-bis(benzyloxy)pyridin-3-yl)-1-methyl-1H-indazol-7-yl) methyl) piperidin-4-yl) methyl) piperazine-1-carboxylate (0.17 g) as an off white solid.

LC-MS (ESI): m/z=717.75 [M+H]⁺.

### Preparation of tert-butyl 4-((1-((3-(2,6-dioxopiperidin-3-yl)-1-methyl-1H-indazol-7-yl) methyl) piperidin-4-yl) methyl) piperazine-1-carboxylate

A stirred solution of tert-butyl 4-((1-((3-(2,6-bis(benzyloxy)pyridin-3-yl)-1-methyl-1H-indazol-7-yl) methyl) piperidin-4-yl) methyl) piperazine-1-carboxylate (0.2 g, 0.27 mmol) in THF (10.0 mL) was purged with N₂. To the mixture was added catalytic amount of acetic acid (0.01 mL) and DMF (0.01 mL). Afterwards, Pd(OH)₂ on carbon (0.25 g, 1.81 mmol) was added, and the reaction mixture was stirred under H₂ (35 psi) pressure for 4 h. The reaction mixture was filtered using celite powder (1.5 g) and washed with ethyl acetate (10 mL). The filtrate was concentrated under vacuum and purified by flash column chromatography (SiO₂, 230-400 mesh; 100% EtOAc) to afford tert-butyl 4-((1-((3-(2,6-dioxopiperidin-3-yl)-1-methyl-1H-indazol-7-yl) methyl) piperidin-4-yl) methyl) piperazine-1-carboxylate (0.08 g) as an off white solid.

LC-MS (ESI): m/z=539.37 [M+H]⁺.

### Preparation of 3-(1-methyl-7-((4-(piperazin-1-ylmethyl) piperidin-1-yl) methyl)-1H-indazol-3-yl) piperidine-2,6-dione

To a solution of tert-butyl 4-((1-((3-(2,6-dioxopiperidin-3-yl)-1-methyl-1H-indazol-7-yl) methyl) piperidin-4-yl) methyl) piperazine-1-carboxylate (0.1 g, 0.18 mmol) in DCM (5.0 mL) at 0° C. was added TFA (1.0 mL). The reaction mixture was stirred at rt for 2 h. The reaction mixture was concentrated under reduced pressure, triturated with n-pentane (10.0 ml), and dried to afford 3-(1-methyl-7-((4-(piperazin-1-ylmethyl) piperidin-1-yl) methyl)-1H-indazol-3-yl) piperidine-2,6-dione (0.07 g) as an off white solid.

LC-MS (ESI): m/z=439.51 [M+H]⁺.

### Preparation of 3-(7-((4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)methyl)-1-methyl-1H-indazol-3-yl)piperidine-2,6-dione

To a stirred solution of 3-(1-methyl-7-((4-(piperazin-1-ylmethyl) piperidin-1-yl) methyl)-1H-indazol-3-yl) piperidine-2,6-dione (0.07 g, 0.15 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.03 g 0.06 mmol) in DMSO (0.7 mL) was added DIPEA (0.14 mL, 0.79 mmol) under inert atmosphere at room temperature. The reaction mixture was stirred at 110° C. for 3 h. The reaction mixture was concentrated under reduced pressure and purified by prep-HPLC to afford 3-(7-((4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)methyl)-1-methyl-1H-indazol-3-yl)piperidine-2,6-dione (0.015 g) as an off white solid.

LC-MS (ESI): m/z=870.06 [M+H]⁺.

¹H NMR (400 MHz, DMSO-d₆): δ 10.88 (s, 1H), 8.83 (s, 1H), 8.22 (d, J=2.00 Hz, 2H), 8.03 (s, 1H), 7.69 (dd, J=2.00, 9.00 Hz, 1H), 7.63 (d, J=8.40 Hz, 1H), 7.43 (d, J=9.20 Hz, 1H), 7.14 (d, J=6.80 Hz, 1H), 7.02-6.98 (m, 1H), 6.20 (br s, 1H), 4.40-4.33 (m, 3H), 4.29 (s, 3H), 3.73-3.72 (m, 2H), 3.56-3.54 (m, 7H), 3.26-3.22 (m, 1H), 2.83-2.81 (m, 2H), 2.68-2.62 (m, 2H), 2.33-2.30 (m, 5H), 2.11-2.03 (m, 2H), 2.03-1.96 (m, 2H), 1.75 (br s, 1H), 1.66-1.63 (m, 2H), 1.53-1.49 (m, 2H), 1.10-1.02 (m, 2H), 0.80-0.65 (m, 1H), 0.6-0.45 (m, 2H), 0.40-0.30 (m, 1H)

### Example 89: 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)piperidin-1-yl)-1-methyl-1H-indazol-3-yl)piperidine-2,6-dione (Compound 194a)

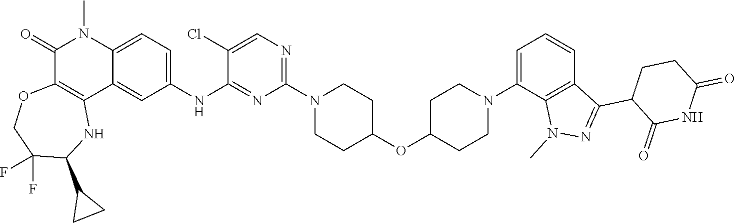 [SMILES: Cn1nc(C2CCC(=O)NC2=O)c2cccc(N3CCC(OC4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-((1-(3-(2,6-bis(benzyloxy)pyridin-3-yl)-1-methyl-1H-indazol-7-yl)piperidin-4-yl)oxy)piperidine-1-carboxylate

To a stirred solution of tert-butyl 4-(piperidin-4-yloxy)piperidine-1-carboxylate (2.0 g, 4.00 mmol) and 3-(2,6-bis(benzyloxy)pyridin-3-yl)-7-bromo-1-methyl-1H-indazole (1.71 g, 2.81 mmol) in dioxane (20 mL) was added cesium carbonate (3.90 g, 12.0 mmol). The reaction mixture degassed with N₂ for 5 min. Afterwards, Pd-PEPPSI-iHeptCl (0.195 g, 0.2 mmol) was added, and the mixture was stirred and heated at 100° C. for 3 h. The reaction mixture was quenched with water and extracted with EtOAc. The combined organic layers were dried over anhydrous sodium sulphate, filtered, concentrated under educed pressure, and purified by flash column chromatography (SiO₂, 100-200, 50% ethyl acetate in petroleum ether) to afford tert-butyl 4-((1-(3-(2,6-bis(benzyloxy)pyridin-3-yl)-1-methyl-1H-indazol-7-yl)piperidin-4-yl)oxy)piperidine-1-carboxylate as a colorless sticky solid (0.35 g).

LC-MS (ESI): m/z=704.47 [M+H]⁺

### Preparation of tert-butyl 4-((1-(3-(2,6-dioxopiperidin-3-yl)-1-methyl-1H-indazol-7-yl)piperidin-4-yl)oxy)piperidine-1-carboxylate

To a solution of tert-butyl 4-((1-(3-(2,6-bis(benzyloxy)pyridin-3-yl)-1-methyl-1H-indazol-7-yl)piperidin-4-yl)oxy)piperidine-1-carboxylate (0.35 g, 0.49 mmol) in tetrahydrofuran (3.5 mL) was added acetic acid (0.009 mL, 0.15 mmol), and Pd(OH)₂ on carbon 20% Pd (0.35 g, w/w). The resulting reaction mixture was stirred under hydrogen atmosphere (15 psi) at room temperature for 16 h. After completion, the reaction mixture was filtered through a celite bed and washed with THF. The combined organic layers were concentrated under reduced pressure and triturated by n-pentane to afford tert-butyl 4-((1-(3-(2,6-dioxopiperidin-3-yl)-1-methyl-1H-indazol-7-yl)piperidin-4-yl)oxy)piperidine-1-carboxylate as a brown sticky solid. (0.21 g).

LC-MS (ESI): m/z=526.38 [M+H]⁺.

### Preparation of 3-(1-methyl-7-(4-(piperidin-4-yloxy)piperidin-1-yl)-1H-indazol-3-yl)piperidine-2,6-dione

To a stirred solution of tert-butyl 4-((1-(3-(2,6-dioxopiperidin-3-yl)-1-methyl-1H-indazol-7-yl)piperidin-4-yl)oxy)piperidine-1-carboxylate (0.35 g, 0.66 mmol) in DCM (3.5 mL) was added TFA (1.75 mL) at 0° C. under a N₂ atmosphere. The resulting reaction mixture was stirred at room temperature for 3 h. The reaction mixture was concentrated under reduced pressure and purified by triturating with diethyl ether followed by n-pentane to afford 3-(1-methyl-7-(4-(piperidin-4-yloxy)piperidin-1-yl)-1H-indazol-3-yl)piperidine-2,6-dione as a brown sticky solid (0.16 g, quantitative).

LC-MS (ESI): m/z=426.30 [M+H]⁺.

### Preparation of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)piperidin-1-yl)-1-methyl-1H-indazol-3-yl)piperidine-2,6-dione

To a solution of 3-(1-methyl-7-(4-(piperidin-4-yloxy)piperidin-1-yl)-1H-indazol-3-yl)piperidine-2,6-dione (0.15 g, 0.287 mmol) in DMSO (1.5 mL) was added DIPEA (0.25 mL, 1.435 mmol).

The reaction mixture stirred for 10 min. (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.053 g, 0.1148 mmol) was then added, and the resulting reaction mixture was heated to 100° C. for 6 h. The reaction mixture was then concentrated under reduced pressure and purified by prep-HPLC to afford 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)piperidin-1-yl)-1-methyl-1H-indazol-3-yl)piperidine-2,6-dione as a light yellow solid (0.022 g).

LC-MS (ESI): m/z=857.46 [M+H]⁺.

¹H NMR (400 MHz, DMSO-d₆): δ 10.84 (s, 1H), 8.82 (s, 1H), 8.44 (bs, 1H), 8.21 (s, 1H), 8.03 (s, 1H), 7.72-7.70 (m, 1H), 7.45-7.42 (d, J=8.8 Hz, 1H), 7.37-7.35 (m, 1H), 7.00-6.99 (m, 2H), 6.21 (s, 1H), 4.49-4.31 (m, 3H), 4.23 (s, 3H), 4.04-4.02 (m, 2H), 3.72 (bs, 1H), 3.56 (s, 4H), 3.26-3.19 (br, 6H), 2.70 (bs, 1H), 2.64-2.60 (m, 2H), 2.31 (m, 1H), 2.18-2.16 (m, 1H), 2.07 (m, 1H), 2.01 (m, 1H), 1.82-1.79 (m, 2H), 1.43-1.32 (m, 4H), 0.69 (m, 1H), 0.53-0.50 (t, 2H), 0.36-0.35 (d, 1H).

### Example 90: 3-(7-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.3]heptan-2-yl)methyl)piperidin-1-yl)-1-methyl-1H-indazol-3-yl)piperidine-2,6-dione (Compound 450a)

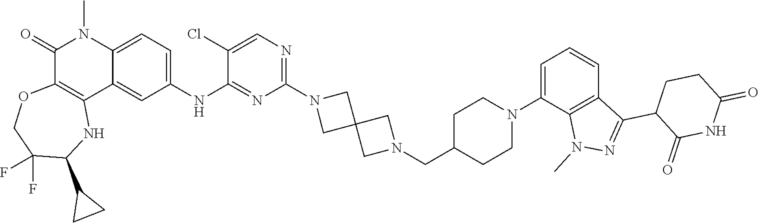 [SMILES: Cn1nc(C2CCC(=O)NC2=O)c2cccc(N3CCC(CN4CC5(C4)CN(c4ncc(Cl)c(Nc6ccc7c(c6)c6c(c(=O)n7C)OCC(F)(F)[C@H](C7CC7)N6)n4)C5)CC3)c21]

### Preparation of (1-(3-(2,6-bis(benzyloxy)pyridin-3-yl)-1-methyl-1H-indazol-7-yl)piperidin-4-yl)methanol

To a stirred solution of 3-(2,6-bis(benzyloxy)pyridin-3-yl)-7-bromo-1-methyl-1H-indazole (2.0 g, 3.99 mmol) in 1,4-dioxane (40 mL) was added 2-(piperidin-4-yl)ethan-1-ol (1.84 g, 15.9 mmol) and cesium carbonate (3.91 g, 12.0 mmol). The reaction was purged with nitrogen for 15 mins and was treated with Pd-PEPPSI-iHeptCl (0.19 g, 0.20 mmol). The resulting mixture was stirred and heated at 100° C. for 16 h. The reaction mixture was then quenched with ice-cold water (30 mL) and extracted with ethyl acetate (3×150 mL). The combined organic layers were washed with brine (30 mL), dried over anhydrous Na₂SO₄, filtered, dried under vacuum, and purified by column chromatography (SiO₂, 100-200 mesh, 55% EtOAc in petroleum ether) to afford (1-(3-(2,6-bis(benzyloxy)pyridin-3-yl)-1-methyl-1H-indazol-7-yl)piperidin-4-yl)methanol (0.80 g) as a yellow solid.

LC-MS (ESI): m/z=535.57 [M+1]⁺

### Preparation of 3-(7-(4-(hydroxymethyl)piperidin-1-yl)-1-methyl-1H-indazol-3-yl)piperidine-2,6-dione

A solution of (1-(3-(2,6-bis(benzyloxy)pyridin-3-yl)-1-methyl-1H-indazol-7-yl)piperidin-4-yl)methanol (0.8 g, 1.49 mmol) in tetrahydrofuran (16 mL) was purged with N₂ for 5 min.

Palladium on activated carbon (10%) (0.80 g, 100% w/w) was added at room temperature. The reaction mixture was then stirred at room temperature for 16 h under H₂ atmosphere (30 psi). The reaction mixture was then filtered through a celite bed, and the filtrate was concentrated under reduced pressure to afford 3-(7-(4-(hydroxymethyl)piperidin-1-yl)-1-methyl-1H-indazol-3-yl)piperidine-2,6-dione (0.50 g) which was used in the next step without further purification.

LC-MS (ESI): m/z=357.39 [M+H]⁺

### Preparation of 1-(3-(2,6-dioxopiperidin-3-yl)-1-methyl-1H-indazol-7-yl)piperidine-4-carbaldehyde

To a solution of 3-(7-(4-(hydroxymethyl)piperidin-1-yl)-1-methyl-1H-indazol-3-yl)piperidine-2,6-dione (0.50 g, 1.40 mmol) in DCM (10 mL) was added Dess-Martin periodinane (1.18 g, 2.80 mmol) at 0° C. under a N₂ atmosphere and stirred at room temperature for 4 h. The reaction mixture was quenched with saturated sodium bicarbonate (20 mL) and extracted with dichloromethane (2×100 mL). The separated organic layers were washed with brine solution (50 mL), dried over anhydrous sodium sulphate, filtered and concentrated to afford 1-(3-(2,6-dioxopiperidin-3-yl)-1-methyl-1H-indazol-7-yl)piperidine-4-carbaldehyde (0.30 g) which was used in the next step without any purification.

LC-MS (ESI): m/z=355.38 [M+H]⁺

### Preparation of tert-butyl 6-((1-(3-(2,6-dioxopiperidin-3-yl)-1-methyl-1H-indazol-7-yl)piperidin-4-yl)methyl)-2,6-diazaspiro[3.3]heptane-2-carboxylate

A stirred solution of 1-(3-(2,6-dioxopiperidin-3-yl)-1-methyl-1H-indazol-7-yl)piperidine-4-carbaldehyde (0.30 g, 0.84 mmol) and tert-butyl 2,6-diazaspiro[3.3]heptane-2-carboxylate (0.33 g, 1.69 mmol) in DCM (10 mL) was purged with N₂ for 1 h. After 1 h, sodium cyanoborohydride (0.10 g, 1.69 mmol) was added at 0° C., and the resulting mixture was stirred for 16 h at room temperature. The reaction mixture was then concentrated under reduced pressure, diluted with water (20 mL), and extracted with ethyl acetate (3×50 mL). The organic layers were concentrated under reduced pressure and purified by trituration with diethyl ether to afford tert-butyl 6-((1-(3-(2,6-dioxopiperidin-3-yl)-1-methyl-1H-indazol-7-yl)piperidin-4-yl)methyl)-2,6-diazaspiro[3.3]heptane-2-carboxylate (0.30 g).

LC-MS (ESI): m/z=537.37 [M+H]⁺.

### Preparation of 3-(7-(4-((2,6-diazaspiro[3.3]heptan-2-yl)methyl)piperidin-1-yl)-1-methyl-1H-indazol-3-yl)piperidine-2,6-dione

To a solution of tert-butyl 6-((1-(3-(2,6-dioxopiperidin-3-yl)-1-methyl-1H-indazol-7-yl)piperidin-4-yl)methyl)-2,6-diazaspiro[3.3]heptane-2-carboxylate (0.3 g, 0.55 mmol) in DCM (3 mL) was added TFA (1.5 mL, 5 v) at 0° C. under N₂ atmosphere. The resultant reaction mixture was stirred at room temperature for 3 h. The reaction mixture was then concentrated under reduced pressure, co-distilled with DCM, and purified by trituration with diethyl ether (2×20 mL) to obtain 3-(7-(4-((2,6-diazaspiro[3.3]heptan-2-yl)methyl)piperidin-1-yl)-1-methyl-1H-indazol-3-yl)piperidine-2,6-dione (0.30 g).

LC-MS (ESI): m/z=437.55 [M+H]⁺

### Preparation of 3-(7-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.3]heptan-2-yl)methyl)piperidin-1-yl)-1-methyl-1H-indazol-3-yl)piperidine-2,6-dione

To a stirred solution of 3-(7-(4-((2,6-diazaspiro[3.3]heptan-2-yl)methyl)piperidin-1-yl)-1-methyl-1H-indazol-3-yl)piperidine-2,6-dione (0.150 g, 0.34 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.17 mmol) in DMSO (3 mL) was added N,N-diisopropylethylamine (0.17 mL, 1.02 mmol) at room temperature. The reaction mixture was heated at 100° C. for 3 h. The reaction mixture was then concentrated under reduced pressure and purified by prep-HPLC to afford 3-(7-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,6-diazaspiro[3.3]heptan-2-yl)methyl)piperidin-1-yl)-1-methyl-1H-indazol-3-yl)piperidine-2,6-dione (0.018 g) as an off white solid.

LC-MS (ESI): m/z=868.60 [M+H]⁺

¹H NMR (400 MHz, DMSO-d₆): δ 10.87 (s, 1H), 8.78 (s, 1H), 8.48 (bs, 1H), 8.26 (d, J=2.0 Hz, 1H), 8.02 (s, 1H), 7.84 (dd, J=9.2, 2.0 Hz, 1H), 7.44 (d, J=9.2 Hz, 1H), 7.02-6.98 (m, 2H), 6.12 (br s, 1H), 4.21 (s, 1H), 4.10-4.02 (m, 4H), 3.32 (s, 3H), 3.31-3.24 (m, 7H), 2.99 (s, 2H), 2.67-2.50 (m, 9H), 1.81-1.78 (m, 2H), 1.50-1.35 (m, 5H), 1.23 (s, 2H), 0.85-0.83 (m, 1H), 0.72-0.69 (m, 1H), 0.57-0.53 (m, 2H), 0.38-0.36 (1H).

### Example 91: 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione (Compound 451a)

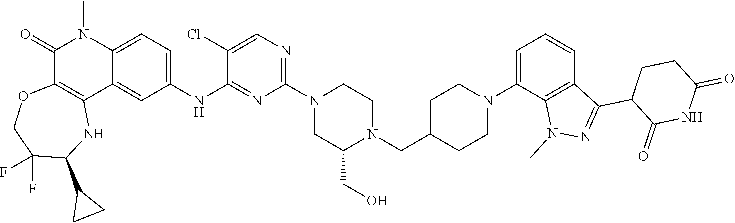 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@H]4CO)CC3)c21]

### Preparation of tert-butyl (S)-4-((1-((benzyloxy)carbonyl)piperidin-4-yl)methyl)-3-(hydroxymethyl)piperazine-1-carboxylate

To a solution of tert-butyl (S)-3-(hydroxymethyl)piperazine-1-carboxylate (2.0 g, 9.25 mmol) and benzyl 4-formylpiperidine-1-carboxylate (2.74 g, 11.10 mmol) in DCM (40 mL) was added acetic acid (0.55 g, 9.25 mmol) at 0° C., and the reaction mixture was stirred for 2 h. NaBH(OAc)3 (3.92 g, 18.50) was then added at 0° C., and the reaction mixture was stirred at room temperature for an additional 16 h. The reaction mixture was quenched with ice-cold water (100 mL) and extracted with DCM (3×100 mL). The combined organic layers were washed with brine (100 mL), dried over anhydrous Na₂SO₄, filtered, dried under vacuum, and purified by column chromatography (SiO₂, 230-400, 25% ethyl acetate in petroleum ether) to afford tert-butyl (S)-4-((1-((benzyloxy)carbonyl)piperidin-4-yl)methyl)-3-(hydroxymethyl)piperazine-1-carboxylate (4.0 g) as a colorless liquid.

LC-MS (ESI): m/z=448.54 [M+H]⁺

### Preparation of tert-butyl (S)-3-(hydroxymethyl)-4-(piperidin-4-ylmethyl)piperazine-1-carboxylate

To a solution of tert-butyl (S)-4-((1-((benzyloxy)carbonyl)piperidin-4-yl)methyl)-3-(hydroxymethyl)piperazine-1-carboxylate (4.0 g, 8.94 mmol) in THF (120 mL) was added 20% Pd(OH)₂ on carbon (4.0 g, 100% w/w). The reaction mixture was stirred under hydrogen atmosphere (70 psi) at room temperature for 16 h. The reaction mixture was filtered through a celite bed, concentrated under reduced pressure, washed with n-pentane, and dried under vacuum to afford tert-butyl (S)-3-(hydroxymethyl)-4-(piperidin-4-ylmethyl)piperazine-1-carboxylate (2.0 g) as brown solid which was used in the next step without purification.

LC-MS (ESI): m/z=314.51 [M+H]⁺

### Preparation of tert-butyl (S)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate

To a stirred solution of 3-(2,6-bis(benzyloxy)pyridin-3-yl)-7-bromo-1-methyl-1H-indazole (0.50 g, 1.00 mmol) and tert-butyl (S)-3-(hydroxymethyl)-4-(piperidin-4-ylmethyl)piperazine-1-carboxylate (0.62 g, 2.00 mmol) in 1,4-dioxane (10 mL) was added cesium carbonate (0.97 g, 3.00 mmol). The reaction mixture was degassed for 10 minutes. Afterwards, Pd PEPPSI iHeptCl (0.04 g, 0.05 mmol) was added, and the resulting mixture was stirred at 100° C. for 12 h. The reaction mixture was then cooled to room temperature, filtered through a celite bed, concentrated under reduced pressure, and purified by flash column chromatography (SiO₂, 100-200, 25% ethyl acetate in petroleum ether) to afford tert-butyl (S)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate as a brown solid (0.4 g).

LC-MS (ESI): m/z=733.84 [M+H]⁺

### Preparation of tert-butyl (3S)-4-((1-(3-(2,6-dioxopiperidin-3-yl)-1-methyl-1H-indazol-7-yl)piperidin-4-yl)methyl)-3-(hydroxymethyl)piperazine-1-carboxylate

To a solution tert-butyl (S)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate (0.40 g, 0.54 mmol) in THF (16 mL) and AcOH (0.40 mL) was added 20% Pd(OH)₂ on carbon (0.40 g, 100% w/w). The reaction mixture was stirred under hydrogen atmosphere (70 psi) for 16 h at room temperature. The reaction mixture was diluted with THF, filtered through a pad of celite, and washed with excess THF in DCM (20%). The filtrate was collected and concentrated to afford tert-butyl (3S)-4-((1-(3-(2,6-dioxopiperidin-3-yl)-1-methyl-1H-indazol-7-yl)piperidin-4-yl)methyl)-3-(hydroxymethyl)piperazine-1-carboxylate as a brown solid (0.30 g) which was used in the next step without further purification.

LC-MS (ESI): m/z=555.71 [M+H]⁺.

### Preparation of 3-(7-(4-(((S)-2-(hydroxymethyl)piperazin-1-yl)methyl)piperidin-1-yl)-1-methyl-1H-indazol-3-yl)piperidine-2,6-dione (TFA salt)

To a solution of tert-butyl (3S)-4-((1-(3-(2,6-dioxopiperidin-3-yl)-1-methyl-1H-indazol-7-yl)piperidin-4-yl)methyl)-3-(hydroxymethyl)piperazine-1-carboxylate (0.30 g, 0.54 mmol) in DCM (3.0 mL) was added TFA (1.5 mL, 5 v). The reaction mixture was stirred for 2 h at room temperature. The reaction mixture was then concentrated and triturated with diethyl ether to obtain 3-(7-(4-(((S)-2-(hydroxymethyl)piperazin-1-yl)methyl)piperidin-1-yl)-1-methyl-1H-indazol-3-yl)piperidine-2,6-dione (0.40 g) as a TFA salt which was used in the next step without further purification.

LC-MS (ESI): m/z=455.52 [M+H]⁺.

### Preparation of 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione

To a stirred solution of 3-(7-(4-(((S)-2-(hydroxymethyl)piperazin-1-yl)methyl)piperidin-1-yl)-1-methyl-1H-indazol-3-yl)piperidine-2,6-dione (TFA salt) (0.20 g, 0.36 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.14 mmol)) in DMSO (4.0 mL) was added DIPEA (0.5 mL, 2.90 mmol). The reaction mixture was heated at 100° C. for 12 h. The reaction mixture was quenched with ice-cold water. The resulting solid was filtered and purified by prep HPLC to afford 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione as an off-white solid (0.030 g).

LC-MS (ESI): m/z=886.54 [M+H]⁺.

¹H NMR (400 MHz, DMSO-d₆): δ 10.87 (s, 1H), 8.82 (s, 1H), 8.32 (s, 1H), 8.19 (s, 1H), 8.04 (s, 1H), 7.76 (d, J=8.00 Hz, 1H), 7.39 (dd, J=32.00, Hz, 2H), 6.18 (s, 1H), 4.46-4.31 (m, 4H), 4.23 (s, 3H), 4.20-4.10 (m, 1H), 3.95-3.85 (m, 1H), 3.64-3.62 (m, 1H), 3.56 (s, 3H), 3.32-3.10 (m, 6H), 2.81 (m, 1H), 2.67-2.60 (m, 5H), 2.32-2.29 (m, 2H), 2.18-2.14 (m, 3H), 1.90 (m, 1H), 1.80 (m, 1H), 1.65 (s, 1H), 1.36-1.30 (m, 3H), 0.70 (m, 1H), 0.51 (d, J=8.00 Hz, 2H), 0.35 (d, J=4.00 Hz, 1H).

### Example 92: 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperazin-1-yl)-1-methyl-1H-indazol-3-yl)piperidine-2,6-dione (Compound 452a)

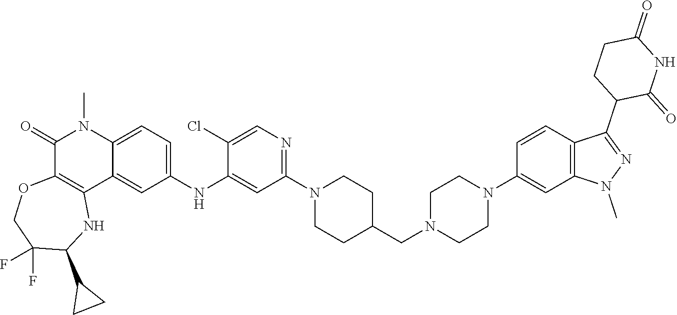 [SMILES: Cn1nc(C2CCC(=O)NC2=O)c2ccc(N3CCN(CC4CCN(c5cc(Nc6ccc7c(c6)c6c(c(=O)n7C)OCC(F)(F)[C@H](C7CC7)N6)c(Cl)cn5)CC4)CC3)cc21]

### Preparation of (S)-10-((5-chloro-2-(4-(hydroxymethyl) piperidin-1-yl) pyridin-4-yl) amino)-2-cyclopropyl-3,3-difluoro-7-methyl-1,2,3,4-tetrahydro-[1,4]oxazepino[2,3-c]quinolin-6(7H)-one

To a solution of (S)-10-((5-chloro-2-fluoropyridin-4-yl)amino)-2-cyclopropyl-3,3-difluoro-7-methyl-1,2,3,4-tetrahydro-[1,4]oxazepino[2,3-c]quinolin-6(7H)-one (0.40 g, 0.88 mmol) in DMSO (4.0 mL) was added piperidin-4-ylmethanol (0.15 g, 1.33 mmol) and DIPEA (1.24 mL, 7.09 mmol). The reaction mixture stirred at 100° C. for 16 h. The reaction mixture was cooled to room temperature, treated with water (20 mL), and extracted with ethyl acetate (3×30 mL). The combined organic layers were dried over anhydrous Na₂SO₄, filtered, concentrated under reduced pressure, and purified by flash chromatography (230-400 silica gel; 70% EtOAc in petroleum ether) to afford (S)-10-((5-chloro-2-(4-(hydroxymethyl) piperidin-1-yl) pyridin-4-yl) amino)-2-cyclopropyl-3,3-difluoro-7-methyl-1,2,3,4-tetrahydro-[1,4]oxazepino[2,3-c]quinolin-6(7H)-one (0.35 g) as a brown solid.

LCMS (ESI): m/z=546.55 [M+H]⁺.

### Preparation of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde

To a solution of (S)-10-((5-chloro-2-(4-(hydroxymethyl) piperidin-1-yl) pyridin-4-yl) amino)-2-cyclopropyl-3,3-difluoro-7-methyl-1,2,3,4-tetrahydro-[1,4]oxazepino[2,3-c]quinolin-6(7H)-one (0.35 g, 0.64 mmol) in DCM (21.0 mL) was added Dess-Martin periodinane (1.08 g, 2.56 mmol) at 0° C. The reaction was stirred at room temperature for 2 h. The reaction mixture was quenched with sodium bicarbonate solution (15 mL) and extracted with DCM (2×15 mL). The combined organic layers were dried over anhydrous Na₂SO₄, filtered, and concentrated under reduced pressure to afford (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde (0.40 g) which was used in the next step without further purification.

LCMS (ESI): m/z=544.24 [M+H]⁺.

### Preparation of tert-butyl 4-(3-(2,6-bis (benzyloxy)pyridin-3-yl)-1-methyl-1H-indazol-6-yl) piperazine-1-carboxylate

To a stirred solution of 3-(2,6-bis(benzyloxy)pyridin-3-yl)-6-bromo-1-methyl-1H-indazole (0.60 g, 1.20 mmol) and tert-butyl piperazine-1-carboxylate (0.56 g, 3.00 mmol) in 1,4-dioxane (18.0 mL, 30 V) was added cesium carbonate (1.17 g, 3.60 mmol). The reaction mixture was degassed with argon for 15 min. Afterwards, Ruphos (0.06 g, 0.12 mmol) was added followed by Ruphos Pd-G3 (0.05 g, 0.06 mmol). The resultant reaction mixture was stirred at 100° C. for 16 h. The reaction mixture was cooled to room temperature, diluted with EtOAc (50 mL), filtered through a celite bed, and washed with 10% MeOH:DCM (150 mL). The filtrate was concentrated and purified by flash chromatography (230-400 silica gel; 10-50% EtOAc in petroleum ether) to afford tert-butyl 4-(3-(2,6-bis (benzyloxy)pyridin-3-yl)-1-methyl-1H-indazol-6-yl) piperazine-1-carboxylate (0.51 g) as a pale yellow solid.

LC-MS (ESI): m/z=606.65[M+H]⁺

### Preparation of tert-butyl 4-(3-(2,6-dioxopiperidin-3-yl)-1-methyl-1H-indazol-6-yl)piperazine-1-carboxylate

To a solution of tert-butyl 4-(3-(2,6-bis (benzyloxy)pyridin-3-yl)-1-methyl-1H-indazol-6-yl) piperazine-1-carboxylate (0.51 g, 0.84 mmol) in THF (30.0 mL, 60 V) was added 20% Pd(OH)₂ on carbon (0.51 g, 100% w/w). The reaction mixture was stirred under hydrogen atmosphere (85 psi) at room temperature for 16 h. The reaction mixture was diluted with THF (40 mL) and filtered through a celite bed and washed with THF:DCM (1:1, 200 mL). The filtrate was concentrated and dried to afford tert-butyl 4-(3-(2,6-dioxopiperidin-3-yl)-1-methyl-1H-indazol-6-yl)piperazine-1-carboxylate (0.35 g) as a black solid which was used in the next step without further purification.

LCMS (ESI): m/z=428.51 [M+H]⁺

### Preparation of 3-(1-methyl-6-(piperazin-1-yl)-1H-indazol-3-yl)piperidine-2,6-dione

To a solution of tert-butyl 4-(3-(2,6-dioxopiperidin-3-yl)-1-methyl-1H-indazol-6-yl)piperazine-1-carboxylate (0.35 g, 0.82 mmol) in DCM (7.0 mL, 20 V) was added TFA (3.5 mL) at 0° C. The reaction mixture was stirred at room temperature for 3 h. The reaction mixture was concentrated under reduced pressure, co-distilled with DCM (2×10 mL), and triturated with diethyl ether (2×10 mL) to afford 3-(1-methyl-6-(piperazin-1-yl)-1H-indazol-3-yl)piperidine-2,6-dione as a brown semi-solid (0.31 g).

LC-MS (ESI): m/z=328.17 [M+H]⁺

### Preparation of 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperazin-1-yl)-1-methyl-1H-indazol-3-yl)piperidine-2,6-dione

To a solution of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde (0.20 g, 0.37 mmol) and 3-(1-methyl-6-(piperazin-1-yl)-1H-indazol-3-yl)piperidine-2,6-dione (0.09 g, 0.29 mmol) in DCM (12.0 mL) was added triethylamine (1.0 mL). The reaction mixture was stirred at 40° C. for 1 h and was then treated with sodium triacetoxyborohydride (0.20 g, 0.91 mmol) at 0° C. The resulting reaction mixture was stirred at room temperature for 4 h. The reaction mixture was concentrated under reduced pressure and purified by prep-HPLC to afford 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperazin-1-yl)-1-methyl-1H-indazol-3-yl)piperidine-2,6-dione (23 mg) as an off white solid.

LC-MS (ESI): m/z=855.52 [M+H]⁺

¹H NMR (400 MHz, DMSO-d6): δ 10.85 (s, 1H), 8.06 (s, 1H), 8.00 (s, 1H), 7.91 (s, 1H), 7.51-7.45 (m, 3H), 6.90 (dd, J=1.60, 9.00 Hz, 1H), 6.82 (d, J=1.20 Hz, 1H), 6.41 (d, J=2.80 Hz, 1H), 6.08 (s, 1H), 4.47-4.40 (m, 2H), 4.25 (q, J=5.20 Hz, 1H), 4.03 (t, J=13.20 Hz, 2H), 3.88 (s, 3H), 3.57 (s, 3H), 3.21 (br s, 5H), 2.68-2.60 (m, 4H), 2.51-2.49 (m, 3H), 2.34-2.24 (m, 1H), 2.18-2.17 (m, 3H), 1.75-1.70 (m, 4H), 1.36-1.28 (m, 1H), 1.09-1.03 (m, 2H), 0.74-0.69 (m, 1H), 0.51-0.48 (m, 2H), 0.31-0.30 (m, 1H).

### Example 93: 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)(methyl)amino)-1-methyl-1H-indazol-3-yl)piperidine-2,6-dione (Compound 454a)

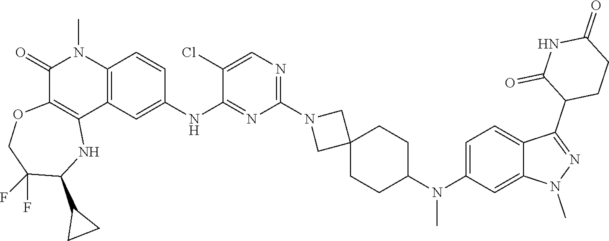 [SMILES: CN(c1ccc2c(C3CCC(=O)NC3=O)nn(C)c2c1)C1CCC2(CC1)CN(c1ncc(Cl)c(Nc3ccc4c(c3)c3c(c(=O)n4C)OCC(F)(F)[C@H](C4CC4)N3)n1)C2]

### Preparation of tert-butyl 7-((3-(2,6-bis(benzyloxy)pyridin-3-yl)-1-methyl-1H-indazol-6-yl)amino)-2-azaspiro[3.5]nonane-2-carboxylate

A stirred solution of 3-(2,6-bis(benzyloxy)pyridin-3-yl)-6-bromo-1-methyl-1H-indazole (0.50 g, 0.99 mmol), tert-butyl 7-amino-2-azaspiro [3.5]nonane-2-carboxylate (0.28 g, 1.19 mmol), and potassium tert-butoxide (0.33 g, 2.99 mmol) in 1,4-dioxane (10 mL) was degassed with argon for 10 minutes. BrettPhos Pd-G3 (0.09 g, 0.01 mmol) was then added, and the reaction mixture was heated at 80° C. for 16 h. The reaction mixture was diluted with ethyl acetate (200 mL) and filtered through a celite bed. The collected filtrate was concentrated under vacuum and purified by flash column chromatography (SiO₂, 230-400 mesh, 40% ethyl acetate in petroleum ether) to afford tert-butyl 7-((3-(2,6-bis(benzyloxy)pyridin-3-yl)-1-methyl-1H-indazol-6-yl)amino)-2-azaspiro[3.5]nonane-2-carboxylate (0.45 g, 25%) as a pale yellow semi-solid.

LC-MS (ESI): m/z=660.43 [M+H]⁺

### Preparation of tert-butyl 7-((3-(2,6-bis(benzyloxy)pyridin-3-yl)-1-methyl-1H-indazol-6-yl)(methyl)amino)-2-azaspiro[3.5]nonane-2-carboxylate

To a solution of tert-butyl 7-((3-(2,6-bis(benzyloxy)pyridin-3-yl)-1-methyl-1H-indazol-6-yl)amino)-2-azaspiro[3.5]nonane-2-carboxylate (0.45 g, 0.65 mmol) in DMF (4.5 mL) was added NaH (0.15 g, 6.53 mmol) at 0° C. The reaction mixture stirred for 30 minutes when iodomethane (0.17 mL, 2.6 mmol) was added. The resulting mixture was stirred at room temperature for an additional 3 h. The reaction mixture was quenched with water and extracted with ethyl acetate.

The organic layer was separated, dried over anhydrous sodium sulfate, filtered, concentrated under reduced pressure, and purified by flash column chromatography (SiO₂, 100-200, 1-5% EtOAc in petroleum ether) to afford tert-butyl 7-((3-(2,6-bis(benzyloxy)pyridin-3-yl)-1-methyl-1H-indazol-6-yl)(methyl)amino)-2-azaspiro[3.5]nonane-2-carboxylate as a brown semi-solid (0.33 g).

LC-MS (ESI): m/z=674.5 [M+H]⁺.

### Preparation of tert-butyl 7-((3-(2,6-dioxopiperidin-3-yl)-1-methyl-1H-indazol-6-yl)(methyl)amino)-2-azaspiro[3.5]nonane-2-carboxylate

To a solution of tert-butyl 7-((3-(2,6-bis(benzyloxy)pyridin-3-yl)-1-methyl-1H-indazol-6-yl)(methyl)amino)-2-azaspiro[3.5]nonane-2-carboxylate (0.33 g, 0.46 mmol) in THF (6.6 mL) was added 20% Pd(OH)₂ on carbon (0.33 g, 100% w/w). The reaction mixture was stirred under hydrogen atmosphere (80 psi) for 16 h at room temperature. The reaction mixture was diluted with THF, filtered through a pad of celite, and washed with an excess amount of THF in DCM (20%).

The filtrate was collected and concentrated to afford tert-butyl 7-((3-(2,6-dioxopiperidin-3-yl)-1-methyl-1H-indazol-6-yl)(methyl)amino)-2-azaspiro[3.5]nonane-2-carboxylate as an off white solid (0.24 g,) which was used in the next step without purification.

LC-MS (ESI): m/z=496.55 [M+H]⁺.

### Preparation of 3-(1-methyl-6-(methyl(2-azaspiro[3.5]nonan-7-yl)amino)-1H-indazol-3-yl)piperidine-2,6-dione

To a solution of tert-butyl 7-((3-(2,6-dioxopiperidin-3-yl)-1-methyl-1H-indazol-6-yl)(methyl)amino)-2-azaspiro[3.5]nonane-2-carboxylate (0.24 g, 0.53 mmol) in DCM (5.34 mL) was added TFA (0.33 mL, 4.31 mmol). The reaction mixture was stirred for 3 h at room temperature. The reaction mixture was concentrated and triturated with diethyl ether to afford 3-(1-methyl-6-(methyl(2-azaspiro[3.5]nonan-7-yl)amino)-1H-indazol-3-yl)piperidine-2,6-dione (0.20 g) which was used in the next step without purification.

LC-MS (ESI): m/z=396.26 [M+H]⁺.

### Preparation of 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)(methyl)amino)-1-methyl-1H-indazol-3-yl)piperidine-2,6-dione

To a solution of 3-(1-methyl-6-(methyl(2-azaspiro[3.5]nonan-7-yl)amino)-1H-indazol-3-yl)piperidine-2,6-dione (0.2 g, 0.50 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.094 g, 0.2 mmol) in DMSO (4 mL) was added N,N-diisopropylethylamine (0.68 mL, 4.0 mmol), and the reaction mixture was stirred at 100° C. for 2 h. The reaction mixture was concentrated under reduced pressure and purified by prep-HPLC to afford 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)(methyl)amino)-1-methyl-1H-indazol-3-yl)piperidine-2,6-dione (0.0243 g) as a yellow solid.

LC-MS (ESI): m/z=827.54 [M+H]⁺

¹H NMR: (400 MHz, DMSO-d₆): δ 10.90 (s, 1H), 8.70 (s, 1H), 8.24 (d, J=2.40 Hz, 1H), 8.03 (s, 1H), 7.83 (d, J=1.60 Hz, 1H), 7.44 (d, J=9.20 Hz, 2H), 6.82 (d, J=11.20 Hz, 1H), 6.59 (s, 1H), 6.15 (s, 1H), 4.43-4.42 (m, 2H), 4.24-4.21 (m, 1H), 3.86 (s, 3H), 3.73 (s, 3H), 3.69-3.57 (m, 5H), 3.32 (s, 1H), 2.76 (s, 3H), 2.67-2.61 (m, 2H), 2.29-2.27 (m, 1H), 2.18-2.16 (m, 1H), 1.94-1.92 (m, 2H), 1.65-1.59 (m, 7H), 0.74-0.72 (m, 1H), 0.54-0.51 (m, 2H), 0.37 (s, 1H).

### Example 94: 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)(methyl)amino)-1-methyl-1H-indazol-3-yl)piperidine-2,6-dione (Compound 455a)

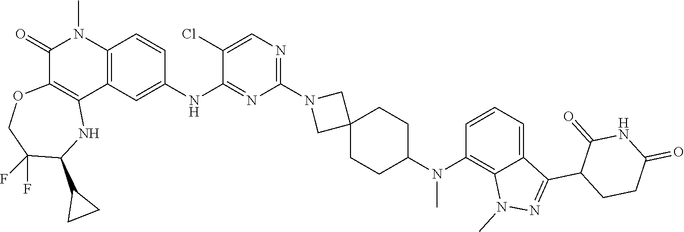 [SMILES: CN(c1cccc2c(C3CCC(=O)NC3=O)nn(C)c12)C1CCC2(CC1)CN(c1ncc(Cl)c(Nc3ccc4c(c3)c3c(c(=O)n4C)OCC(F)(F)[C@H](C4CC4)N3)n1)C2]

### Preparation of tert-butyl 7-((3-(2,6-bis(benzyloxy)pyridin-3-yl)-1-methyl-1H-indazol-7-yl)amino)-2-azaspiro[3.5]nonane-2-carboxylate

A stirred solution of 3-(2,6-bis(benzyloxy)pyridin-3-yl)-7-bromo-1-methyl-1H-indazole (0.50 g, 1.0 mmol), tert-butyl 7-amino-2-azaspiro[3.5]nonane-2-carboxylate (0.28 g, 1.7 mmol), and cesium carbonate (0.97 g, 2.99 mmol) in 1,4-dioxane (10.0 mL) was degassed with argon for 10 minutes. Pd-PEPPSI-IHeptCl (0.09 g, 0.10 mmol) was then added, and the resulting mixture was stirred and heated at 80° C. for 6 h. The reaction mixture was diluted with ethyl acetate (20 mL) and filtered through a celite bed. The collected filtrate was concentrated under reduced pressure and purified by flash column chromatography (SiO₂, 230-400 mesh, 40% ethyl acetate in petroleum ether) to afford tert-butyl 7-((3-(2,6-bis(benzyloxy)pyridin-3-yl)-1-methyl-1H-indazol-7-yl)amino)-2-azaspiro[3.5]nonane-2-carboxylate (0.35 g) as a pale yellow semi-solid.

LC-MS (ESI): m/z=660.75 [M+H]⁺

### Preparation of tert-butyl 7-((3-(2,6-bis(benzyloxy)pyridin-3-yl)-1-methyl-1H-indazol-7-yl)(methyl)amino)-2-azaspiro[3.5]nonane-2-carboxylate

To a solution of tert-butyl 7-((3-(2,6-bis(benzyloxy)pyridin-3-yl)-1-methyl-1H-indazol-7-yl)amino)-2-azaspiro[3.5]nonane-2-carboxylate (0.35 g, 0.53 mmol) in DMF (3.5 mL) was added NaH (0.12 g, 5.3 mmol) at 0° C. The reaction mixture was stirred for 30 minutes, and iodomethane (0.13 mL, 2.12 mmol) was then added. The reaction mixture was stirred at room temperature for an additional 3 h. The reaction mixture was then quenched with water and extracted with ethyl acetate. The organic layer was separated, dried over anhydrous sodium sulfate, filtered, concentrated under vacuum, and purified by flash column chromatography (SiO₂, 100-200, 1-5% EtOAc in petroleum ether) to afford tert-butyl 7-((3-(2,6-bis(benzyloxy)pyridin-3-yl)-1-methyl-1H-indazol-7-yl)(methyl)amino)-2-azaspiro[3.5]nonane-2-carboxylate as a brown semi-solid (0.4 g).

LC-MS (ESI): m/z=674.5 [M+H]⁺.

### Preparation of tert-butyl 7-((3-(2,6-dioxopiperidin-3-yl)-1-methyl-1H-indazol-7-yl)(methyl)amino)-2-azaspiro[3.5]nonane-2-carboxylate

To a solution of tert-butyl 7-((3-(2,6-bis(benzyloxy)pyridin-3-yl)-1-methyl-1H-indazol-7-yl)(methyl)amino)-2-azaspiro[3.5]nonane-2-carboxylate (0.4 g, 0.59 mmol) in THF (8.0 mL) was added 20% Pd(OH)₂ on carbon (0.4 g, 100% w/w). The reaction mixture was stirred under hydrogen atmosphere (80 psi) for 16 h at room temperature. The reaction mixture was diluted with THF, filtered through a pad of celite, and washed with excess THF in DCM (20%). The filtrate was collected and concentrated to afford tert-butyl 7-((3-(2,6-dioxopiperidin-3-yl)-1-methyl-1H-indazol-7-yl)(methyl)amino)-2-azaspiro[3.5]nonane-2-carboxylate as a light brown semi-solid (0.30 g) which was used in the next step without further purification.

LCMS (ESI): m/z=496.6 [M+H]⁺.

### Preparation of 3-(1-methyl-6-(methyl(2-azaspiro[3.5]nonan-7-yl)amino)-1H-indazol-3-yl)piperidine-2,6-dione

To a solution of tert-butyl 7-((3-(2,6-dioxopiperidin-3-yl)-1-methyl-1H-indazol-7-yl)(methyl)amino)-2-azaspiro[3.5]nonane-2-carboxylate (0.30 g, 0.60 mmol) in DCM (6.0 mL) was added TFA (0.36 mL, 4.84 mmol). The reaction mixture was stirred for 3 h at room temperature. The reaction mixture was then concentrated and triturated with diethyl ether to afford 3-(1-methyl-6-(methyl(2-azaspiro[3.5]nonan-7-yl)amino)-1H-indazol-3-yl)piperidine-2,6-dione (0.20 g) which was used in the next step without further purification.

LC-MS (ESI): m/z=396.26 [M+H]⁺

### Preparation of 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)(methyl)amino)-1-methyl-1H-indazol-3-yl)piperidine-2,6-dione

To a stirred solution of 3-(1-methyl-6-(methyl(2-azaspiro[3.5]nonan-7-yl)amino)-1H-indazol-3-yl)piperidine-2,6-dione (0.15 g, 0.37 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.053 g, 0.11 mmol) in DMSO (1.5 mL) was added N, N-diisopropylethylamine (0.54 mL, 3.03 mmol). The reaction mixture was heated at 100° C. for 2 h. The reaction mixture was then concentrated under reduced pressure and purified by prep-HPLC to afford 3-(7-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)(methyl)amino)-1-methyl-1H-indazol-3-yl)piperidine-2,6-dione (0.0821 g) as an off white solid.

LC-MS (ESI): m/z=827.47 [M+H]⁺

¹H NMR (400 MHz, DMSO-d₆): δ 10.90 (s, 1H), 8.73 (s, 1H), 8.22 (s, 1H), 8.01 (s, 1H), 7.81 (d, J=11.20 Hz, 1H), 7.44-7.39 (m, 2H), 7.12 (d, J=7.20 Hz, 1H), 7.04-7.02 (m, 1H), 6.13 (s, 1H), 4.46-4.41 (m, 3H), 4.28 (s, 3H), 3.66 (s, 2H), 3.56 (s, 4H), 3.32 (s, 1H), 2.99 (s, 1H), 2.67-2.62 (m, 4H), 2.34-2.32 (m, 1H), 2.19-2.14 (m, 1H), 1.75 (m, 4H), 1.71-1.72 (m, 2H), 1.44-1.43 (m, 4H), δ 1.32-0.00 (m, 1H), 0.71-0.69 (m, 1H), 0.52-0.51 (m, 2H), 0.35-0.34 (m, 1H).

### Example 95. 3-(6-(4-((9-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3,9-diazaspiro[5.5]undecan-3-yl)methyl)piperidin-1-yl)-1-methyl-1H-indazol-3-yl)piperidine-2,6-dione (Compound 458a)

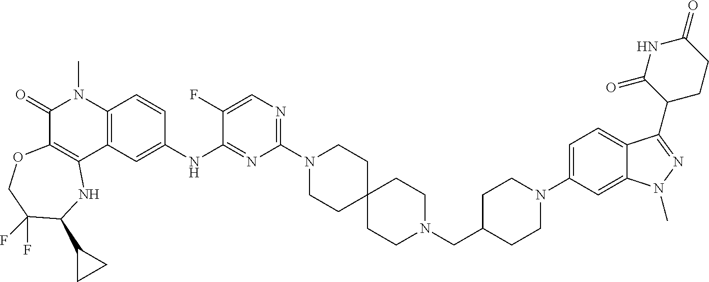 [SMILES: Cn1nc(C2CCC(=O)NC2=O)c2ccc(N3CCC(CN4CCC5(CC4)CCN(c4ncc(F)c(Nc6ccc7c(c6)c6c(c(=O)n7C)OCC(F)(F)[C@H](C7CC7)N6)n4)CC5)CC3)cc21]

### Preparation of (S)-10-amino-2-cyclopropyl-3,3-difluoro-7-methyl-1,2,3,4-tetrahydro-[1,4]oxazepino[2,3-c]quinolin-6(7H)-one

To a stirred solution of (S)-2-cyclopropyl-3,3-difluoro-7-methyl-10-nitro-1,2,3,4-tetrahydro-[1,4]oxazepino[2,3-c]quinolin-6(7H)-one, (1.0 g, 2.84 mmol) in ethanol (40 ml) was added AcOH (0.85 mL, 14.24 mmol) and Zn powder (0.93 g, 14.24 mmol) at 0° C. The resulting mixture was stirred at room temperature for 4 h. The reaction mixture was filtered through a celite pad and washed with ethyl acetate (40 mL). The filtrate was concentrated under reduced pressure and triturated with n-pentane to afford (S)-10-amino-2-cyclopropyl-3,3-difluoro-7-methyl-1,2,3,4-tetrahydro-[1,4]oxazepino[2,3-c]quinolin-6(7H)-one (0.80 g) as a brown solid.

LC-MS (ESI): m/z=322.43 [M+H]⁺

### Preparation of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one

To a solution of (S)-10-amino-2-cyclopropyl-3,3-difluoro-7-methyl-1,2,3,4-tetrahydro-[1,4]oxazepino[2,3-c]quinolin-6(7H)-one (0.80 g, 2.49 mmol) and 2,4-dichloro-5-fluoropyrimidine (1.0 g, 6.22 mmol) in DMSO (8 mL) was added N,N-diisopropylethylamine (2.57 g, 19.92 mmol) at room temperature. The reaction mixture was stirred at 100° C. for 1 h in a microwave reactor. The reaction mixture was quenched with water and extracted with ethyl acetate. The separated organic layers were combined, washed with brine, dried over anhydrous Na₂SO₄, filtered, dried under vacuum, and purified by flash column chromatography (SiO₂, 100-200, 60% ethyl acetate in petroleum ether) to afford (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (0.60 g) as a brown solid.

LC-MS (ESI): m/z=452.41 [M+H]⁺.

### Preparation of 3-(6-(4-((9-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3,9-diazaspiro[5.5]undecan-3-yl)methyl)piperidin-1-yl)-1-methyl-1H-indazol-3-yl)piperidine-2,6-dione

To a solution of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (0.25 g, 0.44 mmol) and 3-(6-(4-((3,9-diazaspiro[5.5]undecan-3-yl)methyl)piperidin-1-yl)-1-methyl-1H-indazol-3-yl)piperidine-2,6-dione (0.54 g, 0.88 mmol) in DMSO (5 mL) was added N,N-diisopropylethylamine (0.57 g, 3.52 mmol). The reaction mixture was stirred at 100° C. for 16 h.

The reaction mixture was concentrated under reduced pressure and purified by prep-HPLC to afford 3-(6-(4-((9-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3,9-diazaspiro[5.5]undecan-3-yl)methyl)piperidin-1-yl)-1-methyl-1H-indazol-3-yl)piperidine-2,6-dione (0.131 g) as an off white solid.

LC-MS (ESI): m/z=908.63 [M+H]⁺

¹H NMR (400 MHz, DMSO-d₆): δ 10.84 (s, 1H), 9.27 (s, 1H), 8.24-8.22 (m, 2H), 7.9-7.98 (d, J=3.60 Hz, 1H), 7.76 (dd, J=6.4, 2 Hz, 1H), 7.50 (t, J=8.80 Hz, 2H), 6.89 (d, J=8.80 Hz, 1H), 6.81 (s, 1H), 6.01 (bs, 1H), 4.41-4.23 (m, 3H), 3.87 (s, 3H), 3.77-3.74 (m, 2H), 3.59-3.56 (m, 6H), 3.15 (m, 1H), 2.74-2.59 (m, 4H), 2.32-2.28 (m, 4H), 2.17-2.15 (m, 2H), 1.79-1.76 (m, 4H), 1.50-1.21 (m, 12H), 0.72 (m, 1H), 0.53-0.51 (m, 2H), 0.37 (m, 1H).

### Example 96: 3-(7-(4-(((R)-4-(5-chloro-4-(((S)-2-cyclopropyl-3,3-difluoro-7-methyl-6-oxo-1,2,3,4,6,7-hexahydro-[1,4]-oxazepino[2,3-c]-quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione (Compound 451b)

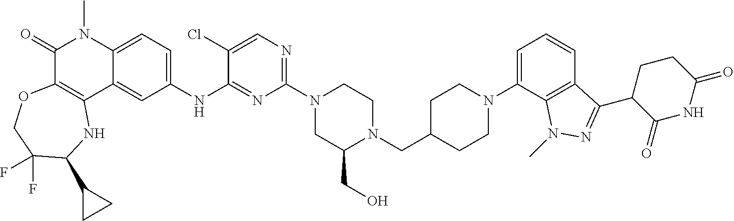 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)C[C@@H]4CO)CC3)c21]

### Preparation of tert-butyl-(R)-4-((1-((benzyloxy)carbonyl)piperidin-4-yl)methyl)-3-(hydroxymethyl)piperazine-1-carboxylate

To a solution of tert-butyl-(R)-3-(hydroxymethyl)piperazine-1-carboxylate (4.00 g, 18.49 mmol), benzyl 4-formylpiperidine-1-carboxylate (4.57 g, 18.49 mmol) and acetic acid (1.06 mL, 18.49 mmol) in DCM (160 mL) was added sodium triacetoxyborohydride (5.88 g, 27.741 mmol) portion wise. The reaction mixture was stirred at room temperature for 18 h, quenched with ice-cold water (200 mL), and extracted with DCM (3×200 mL). The combined organic layers were washed with brine (200 mL), dried over anhydrous Na₂SO₄, filtered, dried under vacuum, and purified by column chromatography (SiO₂; 40% EtOAc in petroleum ether as eluent) to afford tert-butyl-(R)-4-((1-((benzyloxy)carbonyl)piperidin-4-yl)methyl)-3-(hydroxymethyl)piperazine-1-carboxylate (7.90 g) as a colorless semi-solid.

LC-MS (ESI): m/z=448.54 [M+H]⁺

### Preparation of tert-butyl-(R)-3-(hydroxymethyl)-4-(piperidin-4-ylmethyl)piperazine-1-carboxylate

To a solution of tert-butyl-(R)-4-((1-((benzyloxy)carbonyl)piperidin-4-yl)methyl)-3-(hydroxymethyl)piperazine-1-carboxylate (0.78 g, 1.06 mmol) and acetic acid (0.78 mL) in THF (40 mL) was added 20% Pd (OH)₂ on carbon (1.05 g, 7.45 mmol). The reaction mixture was stirred under hydrogen atmosphere (80 psi) at room temperature for 18 h. The reaction mixture was filtered through a celite bed, concentrated under vacuum, washed with n-pentane, and dried to afford tert-butyl-(R)-3-(hydroxymethyl)-4-(piperidin-4-ylmethyl)piperazine-1-carboxylate (0.70 g) as an off white solid.

### Preparation of tert-butyl-(R)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate

A stirred solution of 3-(2,6-bis(benzyloxy)pyridin-3-yl)-7-bromo-1-methyl-1H-indazole (1.40 g, 2.79 mmol), tert-butyl-(R)-3-(hydroxymethyl)-4-(piperidin-4-ylmethyl)piperazine-1-carboxylate (2.63 g, 8.39 mmol), and cesium carbonate (2.73 g, 8.39 mmol) in dioxane (14 mL) was degassed with argon for 5 minutes. The reaction mixture was then treated with Pd-PEPPSI-iHeptCl (0.14 g, 0.14 mmol), degassed with argon for 5 minutes, and heated to 100° C. for 18 h. The reaction mixture was filtered through a celite bed, concentrated under vacuum, and purified by column chromatography (SiO₂, 100-200 mesh; 30-50% EtOAc in petroleum ether as eluent) to afford tert-butyl-(R)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate (0.78 g) as an off white solid.

LC-MS (ESI): m/z=733.79 [M+H]⁺.

### Preparation of tert-butyl-(3R)-4-((1-(3-(2,6-dioxopiperidin-3-yl)-1-methyl-1H-indazol-7-yl)piperidin-4-yl)methyl)-3-(hydroxymethyl)piperazine-1-carboxylate

To a solution of tert-butyl-(R)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate (0.78 g, 1.06 mmol) in THF (47.0 mL), was added acetic acid (0.78 mL) and 20% Pd (OH)₂ on carbon (1.05 g, 7.45 mmol). The reaction mixture was stirred under hydrogen atmosphere (80 psi) at room temperature for 18 h. The reaction mixture was then filtered through a celite bed and washed with THF in DCM (40%, 800 mL). The collected filtrate was concentrated under reduced pressure and triturated with n-pentane (30 mL) to afford tert-butyl-(3R)-4-((1-(3-(2,6-dioxopiperidin-3-yl)-1-methyl-1H-indazol-7-yl)piperidin-4-yl)methyl)-3-(hydroxymethyl)piperazine-1-carboxylate (0.70 g) as a brown solid.

LC-MS (ESI): m/z=555.67 [M+H]⁺

### Preparation of 3-(7-(4-(((R)-2-(hydroxymethyl)piperazin-1-yl)methyl)piperidin-1-yl)-1-methyl-1H-indazol-3-yl)piperidine-2,6-dione

To a solution of tert-butyl-(3R)-4-((1-(3-(2,6-dioxopiperidin-3-yl)-1-methyl-1H-indazol-7-yl)piperidin-4-yl)methyl)-3-(hydroxymethyl)piperazine-1-carboxylate (0.35 g, 0.63 mmol) in DCM (7.0 mL) was added TFA (3.5 mL) at 0° C. The reaction mixture was stirred at room temperature for 1 h. The reaction mixture was then concentrated under reduced pressure and triturated with n-pentane (10 mL) to afford 3-(7-(4-(((R)-2-(hydroxymethyl)piperazin-1-yl)methyl)piperidin-1-yl)-1-methyl-1H-indazol-3-yl)piperidine-2,6-dione (0.40 g) as a brown solid.

LC-MS (ESI): m/z=455.61 [M+H]⁺

### Preparation of 3-(7-(4-(((R)-4-(5-chloro-4-(((S)-2-cyclopropyl-3,3-difluoro-7-methyl-6-oxo-1,2,3,4,6,7-hexahydro-[1,4]-oxazepino[2,3-c]-quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione

To a stirred solution of 3-(7-(4-(((R)-2-(hydroxymethyl)piperazin-1-yl)methyl)piperidin-1-yl)-1-methyl-1H-indazol-3-yl)piperidine-2,6-dione (0.40 g, 0.88 mmol) and (S)-2-cyclopropyl-10-((2,5-dichloropyrimidin-4-yl)amino)-3,3-difluoro-7-methyl-1,2,3,4-tetrahydro-[1,4]-oxazepino[2,3-c]quinolin-6(7H)-one (0.21 g, 0.44 mmol) in DMSO (3.08 mL) was added DIPEA (3.08 mL). The reaction mixture was heated to 100° C. for 18 h. The reaction mixture was concentrated under reduced pressure and purified by prep-HPLC to afford 3-(7-(4-(((R)-4-(5-chloro-4-(((S)-2-cyclopropyl-3,3-difluoro-7-methyl-6-oxo-1,2,3,4,6,7-hexahydro-[1,4]-oxazepino[2,3-c]-quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione (0.087 g) as an off white solid.

LC-MS (ESI): m/z=886.58 [M+H]⁺.

¹H NMR (400 MHz, DMSO-d₆): δ 10.88 (s, 1H), 8.82 (s, 1H), 8.36 (br s, 1H), 8.20 (s, 1H), 8.05 (s, 1H), 7.79 (d, J=7.20 Hz, 1H), 7.42 (d, J=9.20 Hz, 1H), 7.35 (d, J=1.60 Hz, 1H), 7.00-6.98 (m, 1H), 6.20 (br s, 1H), 4.47-4.45 (m, 2H), 4.42-4.41 (m, 2H), 4.24 (s, 3H), 4.13-4.01 (m, 1H), 3.85-3.76 (m, 1H), 3.62 (d, J=7.20 Hz, 1H), 3.56 (s, 3H), 3.22-3.20 (m, 6H), 2.82 (d, J=12.00 Hz, 1H), 2.67-2.67 (m, 3H), 2.62 (d, J=6.00 Hz, 3H), 2.32-2.31 (m, 2H), 2.17-2.15 (m, 3H), 1.90-1.82 (m, 1H), 1.78-1.69 (m, 1H), 1.33-1.30 (m, 2H), 0.71 (d, J=6.40 Hz, 2H), 0.51-0.50 (m, 2H), 0.36-0.35 (m, 1H).

### Example 97: 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione (Compound 459a)

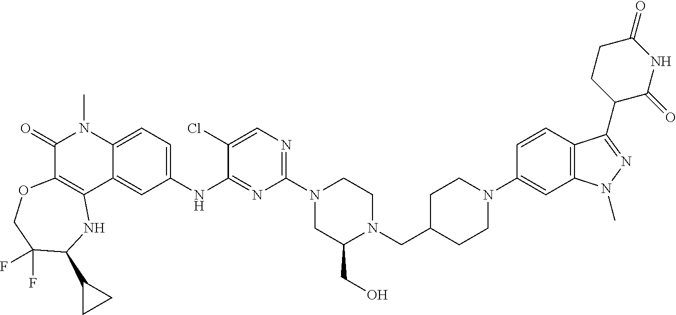 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)C[C@@H]4CO)CC3)cc21]

### Preparation of tert-butyl-(R)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate

To a stirred solution of 3-(2,6-bis(benzyloxy)pyridin-3-yl)-6-bromo-1-methyl-1H-indazole (1.20 g, 2.40 mmol), tert-butyl-(R)-3-(hydroxymethyl)-4-(piperidin-4-ylmethyl)piperazine-1-carboxylate (2.25 g, 7.19 mmol), and NaOtBu (0.69 g, 7.19 mmol) in dioxane (12 mL) was added Brettphos Pd G₃ (0.22 g, 0.24 mmol). The resulting mixture was then stirred and heated at 100° C. for 18 h. The reaction mixture was filtered through a bed of celite, and the filter cake was washed with DCM (200 mL). The filtrate was concentrated under vacuum and purified by flash silica gel column chromatography (75%-80% of ethyl acetate in petroleum ether) to afford tert-butyl-(R)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate (0.35 g) as a pale yellow semi-solid.

LC-MS (ESI): m/z=733.49 [M+H]⁺

### Preparation of tert-butyl-(3R)-4-((1-(3-(2,6-dioxopiperidin-3-yl)-1-methyl-1H-indazol-6-yl)piperidin-4-yl)methyl)-3-(hydroxymethyl)piperazine-1-carboxylate

To a stirred solution of tert-butyl-(R)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate (0.33 g, 0.45 mmol) and AcOH (0.99 mL) in THF (66.0 mL) was added 20% Pd (OH)₂/C (0.63 g, 4.50 mmol).

The resulting mixture was then stirred under a hydrogen atmosphere (80 psi) at ambient temperature for 18 h. The reaction mixture was filtered through a bed of celite and washed with THF. The filtrated was concentrated under reduced pressure, washed with n-pentane, and dried to afford tert-butyl-(3R)-4-((1-(3-(2,6-dioxopiperidin-3-yl)-1-methyl-1H-indazol-6-yl)piperidin-4-yl)methyl)-3-(hydroxymethyl)piperazine-1-carboxylate (0.29 g) as a pale brown semi-solid.

LC-MS (ESI): m/z=555.67 (M+H)+

### Preparation of 3-(6-(4-(((R)-2-(hydroxymethyl)piperazin-1-yl)methyl)piperidin-1-yl)-1-methyl-1H-indazol-3-yl)piperidine-2,6-dione

To a solution of tert-butyl-(3R)-4-((1-(3-(2,6-dioxopiperidin-3-yl)-1-methyl-1H-indazol-6-yl)piperidin-4-yl)methyl)-3-(hydroxymethyl)piperazine-1-carboxylate (0.29 g, 0.52 mmol) in DCM (5.80 mL) was added TFA (1.45 mL). The reaction mixture was stirred at ambient temperature for 1 h. The reaction mixture was then concentrated under reduce pressure, triturated with n-pentane (20 mL) and dried to afford 3-(6-(4-((@-2-(hydroxymethyl)piperazin-1-yl)methyl)piperidin-1-yl)-1-methyl-1H-indazol-3-yl)piperidine-2,6-dione (0.39 g) as a pale brown semi-solid.

LC-MS (ESI): m/z=455.52 [M+H]⁺

### Preparation of 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione

To a stirred solution of 3-(6-(4-(((R)-2-(hydroxymethyl)piperazin-1-yl)methyl)piperidin-1-yl)-1-methyl-1H-indazol-3-yl)piperidine-2,6-dione (0.14 g, 0.31 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.07 g, 0.15 mmol) in DMSO (1.40 mL) was added DIPEA (1.40 mL). The resulting mixture was heated at 100° C. for 18 h. The reaction mixture was concentrated under reduced pressure and purified by prep-HPLC to afford 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione (0.042 g) as an off-white solid.

LC-MS (ESI): m/z=886.54 [M+H]⁺¹H NMR (400 MHz, DMSO-d₆): δ 10.84 (s, 1H), 8.82 (s, 1H), 8.36 (s, 1H), 8.04 (s, 1H), 7.79 (d, J=9.20 Hz, 1H), 7.42 (d, J=9.20 Hz, 1H), 7.47 (d, J=8.80 Hz, 1H), 6.89 (d, J=9.20 Hz, 1H), 6.81 (s, 1H), 6.20 (br s, 1H), 4.42-4.41 (m, 3H), 4.24-4.23 (m, 3H), 3.88 (s, 3H), 3.79-3.76 (m, 3H), 3.56 (br s, 4H), 3.24 (s, 4H), 2.89-2.78 (m, 3H), 2.67-2.64 (m, 3H), 2.32-2.31 (m, 2H), 2.14-2.13 (m, 3H), 1.98-1.65 (m, 4H), 0.65-0.58 (m, 2H), 0.55-0.51 (m, 2H), 0.36-0.32 (m, 1H).

### Example 98: 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione (Compound 459b)

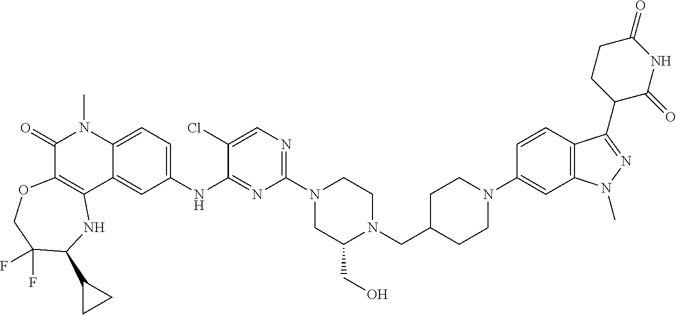 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)C[C@H]4CO)CC3)cc21]

### Preparation of tert-butyl-(S)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate

To stirred solution of 3-(2,6-bis(benzyloxy)pyridin-3-yl)-6-bromo-1-methyl-1H-indazole (0.5 g, 0.99 mmol) and tert-butyl-(S)-3-(hydroxymethyl)-4-(piperidin-4-ylmethyl)piperazine-1-carboxylate (0.49 g, 1.49 mmol) in 1,4-dioxane (10.0 mL) was added cesium carbonate (0.96 g, 2.97 mmol). The reaction mixture was degassed for 10 minutes. Afterwards, Ruphos (0.05 g, 0.09 mmol) and Ruphos-PdG3 (0.04 g, 0.05 mmol) were added, and the resulting solution was stirred at 100° C. for 5 h. The reaction mixture was then cooled to room temperature, filtered through a celite bed, washed with ethyl acetate (100 mL), and concentrated under reduced pressure to obtain a residue, which was purified by flash column chromatography (SiO₂, 230-400 mesh, 25% ethyl acetate in petroleum ether) to afford tert-butyl-(S)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate as a brown semi-solid (0.3 g).

LC-MS (ESI): m/z=733.46 [M+H]⁺

### Preparation of tert-butyl-(3S)-4-((1-(3-(2,6-dioxopiperidin-3-yl)-1-methyl-1H-indazol-6-yl)piperidin-4-yl)methyl)-3-(hydroxymethyl)piperazine-1-carboxylate

To a solution of tert-butyl-(S)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate (0.30 g, 0.41 mmol) in THF (12.0 mL) and AcOH (0.3 mL) was added 20% Pd(OH)₂ on carbon (0.3 g, 100% w/w). The reaction mixture was stirred under hydrogen atmosphere (60 psi) for 16 h at room temperature.

The reaction mixture was diluted with THF and filtered through a pad of celite and washed with 100 mL of 20% THF in DCM. The filtrate was collected and concentrated to afford tert-butyl-(3S)-4-((1-(3-(2,6-dioxopiperidin-3-yl)-1-methyl-1H-indazol-6-yl)piperidin-4-yl)methyl)-3-(hydroxymethyl)piperazine-1-carboxylate as a brown semi-solid (0.24 g) which was used in the next step without further purification.

LC-MS (ESI): m/z=555.57 [M+H]⁺.

### Preparation of 3-(6-(4-(((S)-2-(hydroxymethyl)piperazin-1-yl)methyl)piperidin-1-yl)-1-methyl-1H-indazol-3-yl)piperidine-2,6-dione (TFA salt)

To a solution of tert-butyl-(3S)-4-((1-(3-(2,6-dioxopiperidin-3-yl)-1-methyl-1H-indazol-6-yl)piperidin-4-yl)methyl)-3-(hydroxymethyl)piperazine-1-carboxylate (0.24 g, 0.43 mmol) in DCM (3.0 mL) was added TFA (1.2 mL, 5×). The reaction mixture was stirred for 3 h at room temperature. The reaction mixture was then concentrated and triturated with diethyl ether to obtain 3-(6-(4-(((S)-2-(hydroxymethyl)piperazin-1-yl)methyl)piperidin-1-yl)-1-methyl-1H-indazol-3-yl)piperidine-2,6-dione (0.22 g) as a TFA salt which was used in the next step without purification.

LC-MS (ESI): m/z=455.27 [M+H]⁺.

### Preparation of 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione

To a stirred solution of 3-(6-(4-(((S)-2-(hydroxymethyl)piperazin-1-yl)methyl)piperidin-1-yl)-1-methyl-1H-indazol-3-yl)piperidine-2,6-dione (0.20 g, 0.46 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.18 mmol)) in DMSO (4.0 mL) was added DIPEA (0.6 mL, 3.68 mmol). The reaction mixture was heated at 100° C. for 6 h. The reaction mixture was quenched with ice-cold water, and a solid was collected by filtration and purified by prep-HPLC to afford 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-(hydroxymethyl)piperazin-1-yl)methyl)piperidin-1-yl)-1-methyl-1H-indazol-3-yl)piperidine-2,6-dione as an off-white solid (0.020 g).

LC-MS (ESI): m/z=886.514 [M+H]⁺.

¹H NMR (400 MHz, DMSO-d₆): δ 10.90 (s, 1H), 8.82 (s, 1H), 8.19 (bs, 1H), 8.04 (s, 1H), 7.76 (d, J=8.80 Hz, 1H), 7.47 (d, J=9.20 Hz, 1H), 7.41 (d, J=9.20 Hz, 1H), 6.88 (d, J=1.60 Hz, 1H), 6.81 (bs, 1H), 6.18 (s, 1H), 4.51 (m, 1H), 4.41 (s, 2H), 4.24-4.23 (m, 1H), 4.23 (s, 1H), 3.88 (s, 4H), 3.77 (d, J=12.00 Hz, 2H), 3.59-3.56 (m, 4H), 3.21 (s, 4H), 2.80 (bs, 1H), 2.72-2.70 (m, 1H), 2.62-2.60 (m, 3H), 2.28-2.27 (m, 2H), 2.13-2.12 (m, 3H), 1.79 (s, 1H), 1.72 (s, 3H), 1.26-1.23 (m, 4H), 0.71-0.70 (m, 1H), 0.52-0.51 (m, 2H), 0.36-0.35 (m, 1H).

### Example 99: 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (Compound 460a)

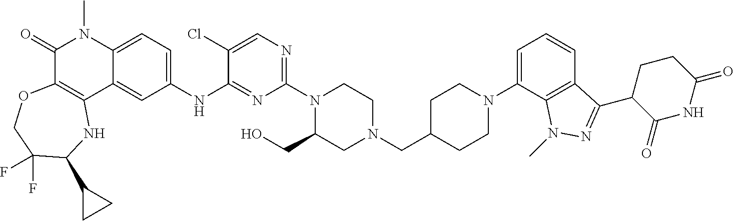 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)[C@H](CO)C4)CC3)c21]

### Preparation of tert-butyl-(S)-4-((1-((benzyloxy)carbonyl)piperidin-4-yl)methyl)-2-(hydroxymethyl)piperazine-1-carboxylate

To a solution of tert-butyl-(S)-2-(hydroxymethyl)piperazine-1-carboxylate (2.0 g, 9.24 mmol), benzyl-4-formylpiperidine-1-carboxylate (2.28 g, 9.24 mmol) and NaBH(OAc)3 (2.35 g, 11.09 mmol) in DCM (40 mL) was added acetic acid (0.5 mL, 9.24 mmol). The reaction mixture was stirred at room temperature for 16 h. The reaction mixture was quenched with ice-cold water (100 mL) and extracted with DCM (3×100 mL). The combined organic layers were then washed with brine (100 mL), dried over anhydrous Na₂SO₄, filtered, dried under vacuum, and purified by column chromatography (SiO2; 40% EtOAc in petroleum ether) to afford tert-butyl-(S)-4-((1-((benzyloxy)carbonyl)piperidin-4-yl)methyl)-2-(hydroxymethyl)piperazine-1-carboxylate (4.0 g) as a white solid.

LC-MS (ESI): m/z=448.32 [M+H]⁺

### Preparation of tert-butyl-(S)-2-(hydroxymethyl)-4-(piperidin-4-ylmethyl)piperazine-1-carboxylate

To a solution of tert-butyl-(S)-4-((1-((benzyloxy)carbonyl)piperidin-4-yl)methyl)-2-(hydroxymethyl)piperazine-1-carboxylate (4.0 g, 8.93 mmol) in THF (240 mL) was added 10% palladium on carbon (4.0 g, 100% w/w). The reaction mixture was stirred under hydrogen atmosphere (60 psi) at room temperature for 16 h. The reaction mixture was filtered through a celite bed, concentrated, washed with n-pentane, and dried to afford tert-butyl-(S)-2-(hydroxymethyl)-4-(piperidin-4-ylmethyl)piperazine-1-carboxylate (2.5 g) as a white solid which was used in the next step without further purification.

LC-MS (ESI): m/z=314.51 [M+H]⁺.

### Preparation of tert-butyl-(S)-4-((1-(3-(2,6-bis(benzyloxy)pyridin-3-yl)-1-methyl-1H-indazol-7-yl)piperidin-4-yl)methyl)-2-(hydroxymethyl)piperazine-1-carboxylate

A solution of 3-(2,6-bis(benzyloxy)pyridin-3-yl)-7-bromo-1-methyl-1H-indazole (0.90 g, 1.79 mmol), tert-butyl-(S)-2-(hydroxymethyl)-4-(piperidin-4-ylmethyl)piperazine-1-carboxylate (1.12 g, 3.59 mmol), and cesium carbonate (1.75 g, 5.39 mmol) in 1,4-dioxane (18 mL) was degassed with argon for 15 minutes. Afterwards, Pd-PEPPSI-IHeptCl (80 mg, 0.09 mmol) was added, and the solution was again degassed for 5 min. The reaction mixture was stirred and heated at 100° C. for 16 h. The reaction mixture was quenched with ice-cold water (50 mL) and extracted with ethyl acetate (3×50 mL). The combined organic layers were washed with brine (50 mL), dried over anhydrous Na₂SO₄, filtered, dried under vacuum, and purified by column chromatography (SiO₂; 40% EtOAc in petroleum ether) to afford tert-butyl-(S)-4-((1-(3-(2,6-bis(benzyloxy)pyridin-3-yl)-1-methyl-1H-indazol-7-yl)piperidin-4-yl)methyl)-2-(hydroxymethyl)piperazine-1-carboxylate (0.71 g) as a pale brown solid.

LC-MS (ESI): m/z=733.7 [M+H]⁺

### Preparation of tert-butyl-(2S)-4-((1-(3-(2,6-dioxopiperidin-3-yl)-1-methyl-1H-indazol-7-yl)piperidin-4-yl)methyl)-2-(hydroxymethyl)piperazine-1-carboxylate

To a solution of tert-butyl-(S)-4-((1-(3-(2,6-bis(benzyloxy)pyridin-3-yl)-1-methyl-1H-indazol-7-yl)piperidin-4-yl)methyl)-2-(hydroxymethyl)piperazine-1-carboxylate (0.71 g, 0.96 mmol) in THF (28.0 mL) was added 20% Pd (OH)₂ on carbon (0.71 g, 100% w/w). The reaction mixture was stirred under hydrogen atmosphere (60 psi) at room temperature for 6 h. The reaction mixture was then filtered through a celite bed, concentrated under vacuum, washed with n-pentane, and dried to afford tert-butyl-(2S)-4-((1-(3-(2,6-dioxopiperidin-3-yl)-1-methyl-1H-indazol-7-yl)piperidin-4-yl)methyl)-2-(hydroxymethyl)piperazine-1-carboxylate (0.52 g) as a green solid.

LC-MS (ESI): m/z=555.67 [M+H]⁺.

### Preparation of 3-(7-(4-(((S)-3-(hydroxymethyl)piperazin-1-yl)methyl)piperidin-1-yl)-1-methyl-1H-indazol-3-yl)piperidine-2,6-dione

To a solution of tert-butyl-(2S)-4-((1-(3-(2,6-dioxopiperidin-3-yl)-1-methyl-1H-indazol-7-yl)piperidin-4-yl)methyl)-2-(hydroxymethyl)piperazine-1-carboxylate (0.52 g, 0.93 mmol) in DCM (5.2 mL) cooled to 0° C. was added TFA (2.6 mL). The reaction mixture was stirred at room temperature for 3 h. The reaction mixture was concentrated under vacuum and washed with n-pentane to afford 3-(7-(4-(((S)-3-(hydroxymethyl)piperazin-1-yl)methyl)piperidin-1-yl)-1-methyl-1H-indazol-3-yl)piperidine-2,6-dione (0.42 g) as a white solid.

LC-MS (ESI): m/z=455.56 [M+H]⁺

### Preparation of 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione

To a solution 3-(7-(4-(((S)-3-(hydroxymethyl)piperazin-1-yl)methyl)piperidin-1-yl)-1-methyl-1H-indazol-3-yl)piperidine-2,6-dione (0.20 g, 0.44 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.22 mmol) in DMSO was added N,N-diisopropylethylamine(0.4 mL, 2.20 mmol). The reaction mixture was stirred at 100° C. for 16 h. The reaction mixture was then poured into ice cold water and stirred for 15 minutes. The resulting precipitate was filtered, dried under vacuum, and purified by prep-HPLC to afford 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (0.023 g) as an off white solid.

LC-MS (ESI): m/z=886.52 [M+H]⁺

¹H NMR (400 MHz, DMSO-d₆): δ 10.92 (s, 1H), 8.80 (s, 1H), 8.22 (s, 1H), 8.04 (s, 1H), 7.77-7.76 (m, 1H), 7.43-7.36 (m, 2H), 7.03-7.00 (m, 2H), 6.21 (br s, 1H), 4.85-4.60 (br s, 1H), 4.32-4.30 (m, 4H), 4.24 (s, 3H), 3.73-3.72 (m, 1H), 3.56 (s, 3H), 3.48-3.47 (m, 1H), 3.32-3.24 (m, 4H), 3.06-2.97 (m, 2H), 2.80-2.79 (m, 1H), 2.67-2.62 (m, 4H), 2.30-2.29 (m, 1H), 2.18-2.16 (m, 3H), 1.91-1.87 (m, 4H), 1.70 (br s, 1H), 1.35-1.32 (m, 3H), 0.71-0.00 (m, 1H), 0.50-0.51 (m, 2H), 0.33-0.35 (m, 1H).

### Example 100: 3-(7-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyridin-2-yl)piperazin-1-yl)methyl)piperidin-1-yl)-1-methyl-1H-indazol-3-yl)piperidine-2,6-dione (Compound 462a)

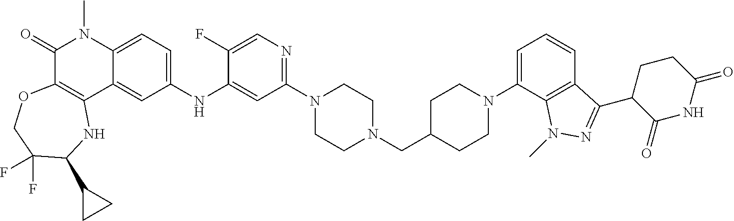 [SMILES: Cn1nc(C2CCC(=O)NC2=O)c2cccc(N3CCC(CN4CCN(c5cc(Nc6ccc7c(c6)c6c(c(=O)n7C)OCC(F)(F)[C@H](C7CC7)N6)c(F)cn5)CC4)CC3)c21]

### Preparation of tert-butyl-4-(5-fluoro-4-iodopyridin-2-yl)piperazine-1-carboxylate

To a solution of 2,5-difluoro-4-iodopyridine (1.00 g, 4.14 mmol) in DMSO (10 mL) and DIPEA (3.67 mL, 20.70 mmol) was added tert-butyl piperazine-1-carboxylate (0.77 g, 4.14 mmol). The reaction mixture was stirred at 100° C. for 16 h. The reaction mixture was cooled to room temperature, diluted with water (40 mL), and extracted with ethyl acetate (20 mL×2). The combined organic layers were dried over anhydrous Na₂SO₄, filtered, concentrated under vacuum, and purified by flash chromatography (230-400 mesh silica gel; 15-20% EtOAc in petroleum ether) to afford tert-butyl-4-(5-fluoro-4-iodopyridin-2-yl)piperazine-1-carboxylate as a white solid (0.50 g). LCMS (ESI): m/z=408.09 [M+H]⁺

### Preparation of tert-butyl-(S)-4-(4-((2-cyclopropyl-3,3-difluoro-7-methyl-6-oxo-1,2,3,4,6,7-hexahydro-[1,4]oxazepino[2,3-c]quinolin-10-yl)amino)-5-fluoropyridin-2-yl)piperazine-1-carboxylate

To a stirred solution of tert-butyl-4-(5-fluoro-4-iodopyridin-2-yl)piperazine-1-carboxylate (0.70 g, 1.65 mmol) and (S)-10-amino-2-cyclopropyl-3,3-difluoro-7-methyl-1,2,3,4-tetrahydro-[1,4]oxazepino[2,3-c]quinolin-6(7H)-one (0.65 g, 2.47 mmol) in 1,4-dioxane (14 mL) was added cesium carbonate (1.61 g, 4.95 mmol). The reaction mixture was degassed with argon for 15 minutes. Afterwards, Ruphos (0.08 g, 0.17 mmol) and Ruphos Pd G3 (0.07 g, 0.08 mmol) were added. The solution was again degassed with argon for 5 minutes and then stirred at 100° C. for 16 h. The reaction mixture was diluted with ethyl acetate (40 mL), filtered through a celite bed, and washed with 10% MeOH:DCM (250 mL). The filtrate was concentrated, dried under vacuum, and purified by flash column chromatography (230-400 mesh silica gel; 70-75% EtOAc in petroleum ether) to afford tert-butyl-(S)-4-(4-((2-cyclopropyl-3,3-difluoro-7-methyl-6-oxo-1,2,3,4,6,7-hexahydro-[1,4]oxazepino[2,3-c]quinolin-10-yl)amino)-5-fluoropyridin-2-yl)piperazine-1-carboxylate (0.40 g) as a white solid.

LCMS (ESI): m/z=601.61 [M+H]⁺.

### Preparation of (S)-2-cyclopropyl-3,3-difluoro-10-((5-fluoro-2-(piperazin-1-yl)pyridin-4-yl)amino)-7-methyl-1,2,3,4-tetrahydro-[1,4]oxazepino[2,3-c]quinolin-6(7H)-one

To a solution of tert-butyl-(S)-4-(4-((2-cyclopropyl-3,3-difluoro-7-methyl-6-oxo-1,2,3,4,6,7-hexahydro-[1,4]oxazepino[2,3-c]quinolin-10-yl)amino)-5-fluoropyridin-2-yl)piperazine-1-carboxylate (0.40 g, 0.66 mmol) in DCM (4 mL,) was added TFA (2 mL) at 0° C. The reaction mixture was stirred at room temperature for 3 h. The reaction mixture was concentrated under reduced pressure, co-distilled with DCM (3×10 mL), and dried to afford (S)-2-cyclopropyl-3,3-difluoro-10-((5-fluoro-2-(piperazin-1-yl)pyridin-4-yl)amino)-7-methyl-1,2,3,4-tetrahydro-[1,4]oxazepino[2,3-c]quinolin-6(7H)-one (0.45 g) as a brown solid which was used in the next step without further purification.

LC-MS (ESI): m/z=501.25 [M+H]⁺.

### Preparation of (1-(3-(2,6-bis(benzyloxy)pyridin-3-yl)-1-methyl-1H-indazol-7-yl)piperidin-4-yl)methanol

To a stirred solution of 3-(2,6-bis(benzyloxy)pyridin-3-yl)-7-bromo-1-methyl-1H-indazole (1.0 g, 1.99 mmol) and piperidin-4-ylmethanol (0.92 g, 7.99 mmol) in 1,4-dioxane (20 mL) was added cesium carbonate (1.95 g, 5.99 mmol). The reaction mixture was degassed with argon for 10 minutes. Afterwards, Pd-PEPPSI-IHeptCl (0.097 g, 0.10 mmol) was added, and the solution was again degassed for 5 min. The reaction mixture was then stirred at 100° C. for 16 h. The reaction mixture was cooled to room temperature, diluted with ethyl acetate, and filtered through a celite bed. The filtrate was then concentrated under reduced pressure and purified by flash column chromatography (SiO₂, 230-400 mesh, 30-35% ethyl acetate in petroleum ether) to afford (1-(3-(2,6-bis(benzyloxy)pyridin-3-yl)-1-methyl-1H-indazol-7-yl)piperidin-4-yl)methanol (0.32 g) as a brown sticky compound.

LCMS (ESI): m/z=535.60 [M+H]⁺

### Preparation of 3-(7-(4-(hydroxymethyl)piperidin-1-yl)-1-methyl-1H-indazol-3-yl)piperidine-2,6-dione

To a solution of (1-(3-(2,6-bis(benzyloxy)pyridin-3-yl)-1-methyl-1H-indazol-7-yl)piperidin-4-yl)methanol (0.30 g, 0.56 mmol) in THF (12 mL) was added 20% Pd(OH)₂ on carbon (0.30 g, 100% w/w). The reaction was stirred at room temperature under H₂ atmosphere (60 psi) for 5 h.

The reaction mixture was diluted with THF (50 mL), filtered through a celite bed, and washed with 200 mL of THF:DCM (1:1). The filtrate was concentrated and dried under vacuum to afford 3-(7-(4-(hydroxymethyl)piperidin-1-yl)-1-methyl-1H-indazol-3-yl)piperidine-2,6-dione (0.21 g) as a brown solid which was used in the next step without further purification.

LCMS (ESI): m/z=357.19 [M+H]⁺.

### Preparation of 1-(3-(2,6-dioxopiperidin-3-yl)-1-methyl-1H-indazol-7-yl)piperidine-4-carbaldehyde

To a solution of 3-(7-(4-(hydroxymethyl)piperidin-1-yl)-1-methyl-1H-indazol-3-yl)piperidine-2,6-dione (0.20 g, 0.56 mmol) in DCM (20 mL) was added Dess-Martin periodinane (0.71 g, 1.68 mmol) at 0° C. The reaction mixture was stirred at room temperature for 4 h. The reaction mixture was extracted with DCM (50 mL×2), dried over anhydrous Na₂SO₄, filtered, and concentrated under reduced pressure to afford 1-(3-(2,6-dioxopiperidin-3-yl)-1-methyl-1H-indazol-7-yl)piperidine-4-carbaldehyde as a brown gummy compound (0.25 g) which was used in the next step without further purification.

LC-MS (ESI): m/z=355.46 [M+H]⁺.

### Preparation of 3-(7-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyridin-2-yl)piperazin-1-yl)methyl)piperidin-1-yl)-1-methyl-1H-indazol-3-yl)piperidine-2,6-dione

To a stirred solution of 1-(3-(2,6-dioxopiperidin-3-yl)-1-methyl-1H-indazol-7-yl)piperidine-4-carbaldehyde (0.1 g, 0.28 mmol) and (S)-2-cyclopropyl-3,3-difluoro-10-((5-fluoro-2-(piperazin-1-yl)pyridin-4-yl)amino)-7-methyl-1,2,3,4-tetrahydro-[1,4]oxazepino[2,3-c]quinolin-6(7H)-one (0.084 g, 0.16 mmol) in DCM (2 mL, 20 V), was added TEA (0.50 mL). The reaction mixture was refluxed for 1 h. Then the reaction mixture was cooled to room temperature, and sodium triacetoxyborohydride (0.17 g, 0.84 mmol) was added at 0° C. The resulting mixture was stirred at room temperature for 5 h. The reaction mixture was concentrated under reduced pressure and purified by prep-HPLC to afford 3-(7-(4-((4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyridin-2-yl)piperazin-1-yl)methyl)piperidin-1-yl)-1-methyl-1H-indazol-3-yl)piperidine-2,6-dione (0.028 g) as an off white solid.

LC-MS (ESI): m/z=839.55 [M+H]⁺

¹H NMR (400 MHz, DMSO-d6): δ 10.88 (s, 1H), 8.44 (s, 1H), 8.04 (s, 1H), 7.90 (d, J=2.80 Hz, 1H), 7.47 (d, J=9.20 Hz, 1H), 7.43 (d, J=1.60 Hz, 1H), 7.36 (dd, J=2.80, 6.60 Hz, 1H), 7.03-6.99 (m, 2H), 6.42 (m, 1H), 6.25 (d, J=6.00 Hz, 1H), 4.44-4.31 (m, 3H), 4.23 (s, 3H), 3.57 (s, 3H), 3.32-3.23 (m, 7H), 2.67-2.58 (m, 4H), 2.46-2.37 (m, 4H), 2.36-2.10 (m, 4H), 1.92-1.80 (m, 2H), 1.74-1.60 (m, 1H), 1.46-1.26 (m, 3H), 0.72-0.71 (m, 1H), 0.52-0.51 (m, 2H), 0.32-0.31 (m, 1H).

### Example 101: 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazin-1-yl)methyl)piperidin-1-yl)-1-methyl-1H-indazol-3-yl)piperidine-2,6-dione (Compound 463a)

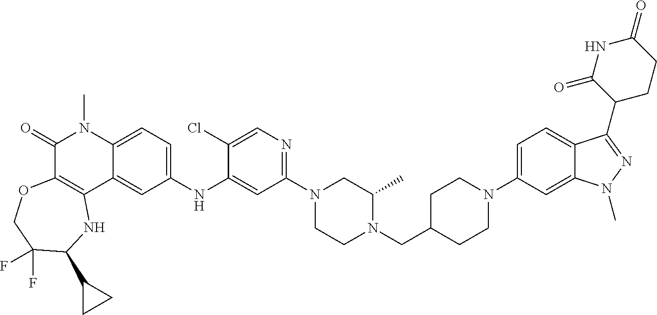 [SMILES: C[C@H]1CN(c2cc(Nc3ccc4c(c3)c3c(c(=O)n4C)OCC(F)(F)[C@H](C4CC4)N3)c(Cl)cn2)CCN1CC1CCN(c2ccc3c(C4CCC(=O)NC4=O)nn(C)c3c2)CC1]

### Preparation of (1-(3-(2,6-bis(benzyloxy)pyridin-3-yl)-1-methyl-1H-indazol-6-yl)piperidin-4-yl)methanol

To a stirred solution of 3-(2,6-bis(benzyloxy)pyridin-3-yl)-6-bromo-1-methyl-1H-indazole (2.0 g, 3.99 mmol) in 1,4-dioxane (40 mL) was added piperidin-4-ylmethanol (1.84 g, 15.9 mmol) and cesium carbonate (3.91 g, 12.0 mmol). The reaction mixture was purged with nitrogen for 15 minutes. Afterwards, Pd-PEPPSI-iHeptCl (0.19 g, 0.20 mmol) was added, and the resulting solution was heated at 100° C. for 16 h. After completion, the reaction was quenched with ice-cold water (30 mL) and extracted with ethyl acetate (3×150 mL). The combined organic layers were washed with brine (30 mL), dried over anhydrous Na₂SO₄, filtered, and dried under vacuum to get a residue which was purified by column chromatography (SiO₂, 100-200 mesh, 80% EtOAc in petroleum ether) to afford (1-(3-(2,6-bis(benzyloxy)pyridin-3-yl)-1-methyl-1H-indazol-6-yl)piperidin-4-yl)methanol (1.80 g) as a yellow liquid.

LC-MS (ESI): m/z=535.38 [M+H]⁺

### Preparation of 3-(6-(4-(hydroxymethyl)piperidin-1-yl)-1-methyl-1H-indazol-3-yl)piperidine-2,6-dione

To a stirred solution of (1-(3-(2,6-bis(benzyloxy)pyridin-3-yl)-1-methyl-1H-indazol-6-yl)piperidin-4-yl)methanol (1.8 g, 3.36 mmol) in tetrahydrofuran (16 mL) under N₂ was added palladium on activated carbon (10%) (1.80 g, 100% w/w) at room temperature. The reaction mixture was then stirred at room temperature for 16 h with a H₂ balloon (15 psi). After completion, the reaction was filtered through a celite bed, and the filtrate was concentrated under reduced pressure to afford 3-(6-(4-(hydroxymethyl)piperidin-1-yl)-1-methyl-1H-indazol-3-yl)piperidine-2,6-dione (0.60 g) which was used in the next step without any purification.

LC-MS (ESI): m/z=357.48 [M+H]⁺

### Preparation of 1-(3-(2,6-dioxopiperidin-3-yl)-1-methyl-1H-indazol-6-yl)piperidine-4-carbaldehyde

To a solution of 3-(6-(4-(hydroxymethyl)piperidin-1-yl)-1-methyl-1H-indazol-3-yl)piperidine-2,6-dione (0.30 g, 0.84 mmol) in DCM (10 mL) under N₂ atmosphere was added Dess-Martin periodinane (0.71 g, 1.68 mmol) at 0° C. The reaction mixture was stirred at room temperature for 4 h. After completion, the reaction was quenched with a saturated solution of sodium bicarbonate (20 mL) and extracted with dichloromethane (2×100 mL). The organic layer was separated, washed with brine (50 mL), dried over anhydrous sodium sulphate, filtered, and concentrated to afford 1-(3-(2,6-dioxopiperidin-3-yl)-1-methyl-1H-indazol-6-yl)piperidine-4-carbaldehyde (0.30 g) which was used in the next step without further purification.

LC-MS (ESI): m/z=355.38 [M+H]⁺

### Preparation of tert-butyl-(S)-4-(5-chloro-4-iodopyridin-2-yl)-2-methylpiperazine-1-carboxylate

To a solution of tert-butyl (S)-2-methylpiperazine-1-carboxylate (2.0 g, 9.98 mmol) in DMSO (40 mL) DIPEA (8.8 mL, 49.93 mmol) and 5-chloro-2-fluoro-4-iodopyridine (2.31 g, 8.98 mmol) was added at room temperature under N₂ atmosphere. Then, the reaction mixture was stirred for 3 h at 120° C. After completion, the reaction mixture was diluted with 100 mL ice water and extracted with ethyl acetate (75 mL×3). The organic layer was concentrated and purified by column chromatography (SiO₂, 100-200 mesh, 4.0% EtOAc in petroleum ether) to afford tert-butyl-(S)-4-(5-chloro-4-iodopyridin-2-yl)-2-methylpiperazine-1-carboxylate (1.23 g).

LC-MS (ESI): m/z=438.2 [M+H]⁺

### Preparation of tert-butyl-(S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazine-1-carboxylate

To a stirred solution of tert-butyl-(S)-4-(5-chloro-4-iodopyridin-2-yl)-2-methylpiperazine-1-carboxylate (1.23 g, 2.810 mmol) in 1,4-dioxane (24 mL) was added (S)-10-amino-2-cyclopropyl-3,3-difluoro-7-methyl-1,2,3,4-tetrahydro-[1,4]oxazepino [2,3-c]quinolin-6(7H)-one (0.993 g, 3.091 mmol) and cesium carbonate (3.91 g, 12.0 mmol) under nitrogen gas. RuPhos (0.131 g, 0.281 mmol) and RuPhos-Pd-G3 (0.117 g, 0.141 mmol) were added thereafter, and the reaction mixture was heated at 100° C. for 16 h. After completion, the reaction mixture was quenched with ice-cold water (30 mL) and extracted with ethyl acetate (3×150 mL). The combined organic layers were washed with brine (30 mL), dried over anhydrous Na₂SO₄, filtered, and dried under vacuum to get a residue which was purified by column chromatography (SiO₂, 100-200 mesh, 30% EtOAc in petroleum ether) to afford tert-butyl-(S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazine-1-carboxylate (1.20 g) as a yellow solid.

LC-MS (ESI): m/z=631.13 [M+H]⁺

### Preparation of (S)-10-((5-chloro-2-((S)-3-methylpiperazin-1-yl)pyridin-4-yl)amino)-2-cyclopropyl-3,3-difluoro-7-methyl-1,2,3,4-tetrahydro-[1,4]oxazepino[2,3-c]quinolin-6(7H)-one

To a solution of tert-butyl-(S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazine-1-carboxylate (1.2 g, 1.901 mmol) in DCM (24 mL) was added TFA (18 mL) at 0° C. under N₂ atmosphere, and the resultant reaction mixture was stirred at room temperature for 3 h. The reaction mixture was concentrated under reduced pressure, co-distilled with DCM, and purified by trituration with diethyl ether (2×20 mL) to afford (S)-10-((5-chloro-2-((S)-3-methylpiperazin-1-yl)pyridin-4-yl)amino)-2-cyclopropyl-3,3-difluoro-7-methyl-1,2,3,4-tetrahydro-[1,4]oxazepino[2,3-c]quinolin-6(7H)-one (1.0 g).

LC-MS (ESI): m/z=531.46 [M+H]⁺

### Preparation of 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazin-1-yl)methyl)piperidin-1-yl)-1-methyl-1H-indazol-3-yl)piperidine-2,6-dione

To a solution of (S)-10-((5-chloro-2-((S)-3-methylpiperazin-1-yl)pyridin-4-yl)amino)-2-cyclopropyl-3,3-difluoro-7-methyl-1,2,3,4-tetrahydro-[1,4]oxazepino[2,3-c]quinolin-6(7H)-one (0.22 g, 0.415 mmol) in THF (2.2 mL,), was added acetic acid (0.025 ml), sodium acetate (0.102 g, 1.245 mmol), and 1-(3-(2,6-dioxopiperidin-3-yl)-1-methyl-1H-indazol-6-yl)piperidine-4-carbaldehyde (0.191 g, 0.539 mmol). The reaction mixture was stirred for 2 h at room temperature. To this reaction mixture, sodium triacetoxyborohydride (0.264 g, 1.245 mmol) was added and stirred at room temperature for 16 h. The reaction mixture was concentrated under reduced pressure and purified by prep-HPLC to afford 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazin-1-yl)methyl)piperidin-1-yl)-1-methyl-1H-indazol-3-yl)piperidine-2,6-dione (0.018 g) as an off white solid.

LC-MS (ESI): m/z=869.55 [M+H]⁺

¹H NMR (400 MHz, DMSO-d₆): δ 10.83 (s, 1H), 8.05 (d, J=6 Hz, 2H), 7.92 (s, 1H), 7.50-7.45 (m, 3H), 6.88 (d, J=8.8 Hz, 1H), 6.80 (s, 1H), 6.42 (bs, 1H), 6.08 (s, 1H), 4.40-4.25 (m, 2H), 4.24-4.22 (m, 1H), 3.87 (s, 3H), 3.76 (d, J=12 Hz, 2H), 3.65 (d, J=10.4 Hz, 1H), 3.573-3.508 (m, 3H), 3.34-3.17 (m, 1H) 2.9-2.72 (m, 2H), 2.68-2.5 (m, 5H), 2.49-2.32 (m, 2H), 2.32-2.12 (m, 3H), 1.72 (d, J=2.8 Hz, 1H), 1.68-1.50 (m, 4H), 1.35-1.23 (m, 3H), 0.96-0.95 (m, 3H), 0.80-0.75 (m, 1H), 0.60-0.55 (m, 2H), 0.40-0.31 (m, 1H).

### Example 102: 4-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)-2-(4-((1-(3-(2,6-dioxopiperidin-3-yl)-1-methyl-1H-indazol-7-yl)piperidin-4-yl)methyl)piperazin-1-yl)pyrimidine-5-carbonitrile (Compound 441a)

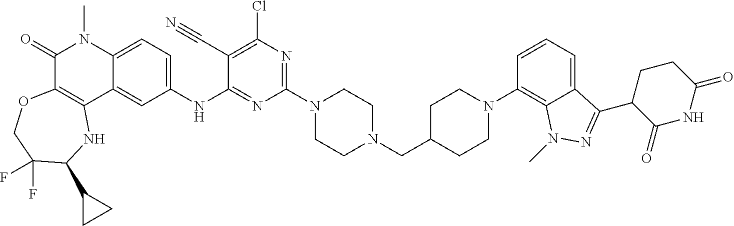 [SMILES: Cn1nc(C2CCC(=O)NC2=O)c2cccc(N3CCC(CN4CCN(c5nc(Cl)c(C#N)c(Nc6ccc7c(c6)c6c(c(=O)n7C)OCC(F)(F)[C@H](C7CC7)N6)n5)CC4)CC3)c21]

### Preparation of (S)-2,4-dichloro-6-((2-cyclopropyl-3,3-difluoro-7-methyl-6-oxo-1,2,3,4,6,7-hexahydro-[1,4]oxazepino[2,3-c]quinolin-10-yl)amino)pyrimidine-5-carbonitrile

To a solution of (S)-10-amino-2-cyclopropyl-3,3-difluoro-7-methyl-1,2,3,4-tetrahydro-[1,4]oxazepino[2,3-c]quinolin-6(7H)-one (0.20 g, 0.62 mmol) in DMSO (2.0 mL) was added 2,4,6-trichloropyrimidine-5-carbonitrile (0.07 g, 0.37 mmol) and DIPEA (0.34 mL, 1.86 mmol). The reaction mixture was heated to 100° C. for 3 h. The reaction mixture was concentrated under reduced pressure and purified by prep-HPLC to afford (S)-2,4-dichloro-6-((2-cyclopropyl-3,3-difluoro-7-methyl-6-oxo-1,2,3,4,6,7-hexahydro-[1,4]oxazepino[2,3-c]quinolin-10-yl)amino)pyrimidine-5-carbonitrile as an off white solid (0.15 g).

LC-MS (ESI): m/z=493.42 [M+H]⁺

### Preparation of 4-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)-2-(4-((1-(3-(2,6-dioxopiperidin-3-yl)-1-methyl-1H-indazol-7-yl)piperidin-4-yl)methyl)piperazin-1-yl)pyrimidine-5-carbonitrile

To a stirred solution (S)-2,4-dichloro-6-((2-cyclopropyl-3,3-difluoro-7-methyl-6-oxo-1,2,3,4,6,7-hexahydro-[1,4]oxazepino[2,3-c]quinolin-10-yl)amino)pyrimidine-5-carbonitrile (0.20 g, 0.304 mmol) in DMSO (2.0 mL) was added 3-(1-methyl-7-(4-(piperazin-1-ylmethyl)piperidin-1-yl)-1H-indazol-3-yl)piperidine-2,6-dione (0.07 g, 0.187 mmol) and DIPEA (0.34 mL, 1.86 mmol).

The reaction mixture was heated to 100° C. for 8 h. The reaction mixture was concentrated under reduced pressure and purified by prep-HPLC to afford 4-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)-2-(4-((1-(3-(2,6-dioxopiperidin-3-yl)-1-methyl-1H-indazol-7-yl)piperidin-4-yl)methyl)piperazin-1-yl)pyrimidine-5-carbonitrile (0.05 g).

LC-MS (ESI): m/z=881.52 [M+H]⁺

¹H NMR (400 MHz, DMSO-d₆): δ 10.82 (s, 1H), 10.52 (s, 1H), 8.25 (s, 1H), 7.58 (dd, J=1.60, 9.00 Hz, 1H), 7.45 (dd, J=9.20, Hz, 1H), 7.36-7.36 (m, 1H), 7.00-6.99 (m, 2H), 6.09 (s, 1H), 4.31-4.33 (m, 3H), 4.24 (s, 3H), 3.85 (s, 3H), 3.55 (s, 3H), 3.26 (s, 3H), 2.61-2.62 (m, 4H), 2.50-2.50 (m, 5H), 2.30-2.31 (m, 2H), 2.18 (s, 1H), 1.85-1.88 (m, 2H), 1.77-1.75 (m, 2H), 1.30-1.31 (m, 3H), 0.70-0.0.69 (m, 1H), 0.52-0.53 (m, 2H), 0.35 (s, 1H).

### Example 103: 3-(6-(((6S, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 464a)

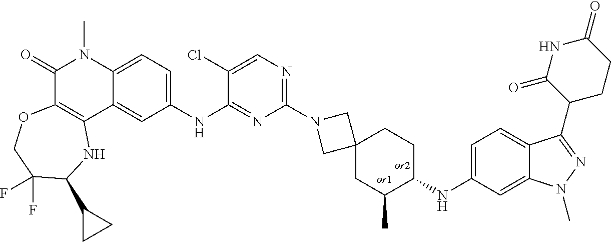 [SMILES: C[C@H]1CC2(CC[C@@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of tert-butyl-(6S, 7S)-7-((3-(2,6-dioxopiperidin-3-yl)-1-methyl-1H-indazol-6-yl)amino)-6-methyl-2-azaspiro[3.5]nonane-2-carboxylate

To a solution of tert-butyl-(6S, 7S)-7-((3-(2,6-bis(benzyloxy)pyridin-3-yl)-1-methyl-1H-indazol-6-yl)amino)-6-methyl-2-azaspiro[3.5]nonane-2-carboxylate (0.80 g, 1.18 mmol) in THF (80.0 mL) was added 20% Pd(OH)₂ on carbon (0.80 g, 100% w/w). The reaction mixture was stirred under hydrogen atmosphere (85 psi) at room temperature for 16 h. The reaction mixture was diluted with THF (50 mL) and filtered through a celite bed and washed with THF:DCM (1:1, 200 mL). The filtrate was concentrated and dried to afford tert-butyl-(6S, 7S)-7-((3-(2,6-dioxopiperidin-3-yl)-1-methyl-1H-indazol-6-yl)amino)-6-methyl-2-azaspiro[3.5]nonane-2-carboxylate (0.66 g) as a black solid which was used in the next step without further purification.

LCMS (ESI): m/z=496.35 [M+H]⁺

### Preparation of 3-(1-methyl-6-(((6S, 7S)-6-methyl-2-azaspiro[3.5]nonan-7-yl)amino)-1H-indazol-3-yl)piperidine-2,6-dione

To a solution of tert-butyl-(6S, 7S)-7-((3-(2,6-dioxopiperidin-3-yl)-1-methyl-1H-indazol-6-yl)amino)-6-methyl-2-azaspiro[3.5]nonane-2-carboxylate (0.65 g, 1.31 mmol) in DCM (13.0 mL) was added TFA (6.5 mL, 10 V) at 0° C. The resultant reaction mixture was stirred at room temperature for 3 h. The reaction mixture was concentrated under reduced pressure, co-distilled with DCM (2×15 mL), and triturated with diethyl ether (2×10 mL) to afford 3-(1-methyl-6-(((6S, 7S)-6-methyl-2-azaspiro[3.5]nonan-7-yl)amino)-1H-indazol-3-yl)piperidine-2,6-dione as a brown semi-solid (0.60 g).

LC-MS (ESI): m/z=396.56 [M+H]⁺

### Preparation of 3-(6-(((6S, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a solution of 3-(1-methyl-6-(((6S, 7S)-6-methyl-2-azaspiro[3.5]nonan-7-yl)amino)-1H-indazol-3-yl)piperidine-2,6-dione (0.30 g, 0.76 mmol) in DMSO (3.0 mL) was added DIPEA (3.0 mL) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.14 g, 0.30 mmol). The reaction mixture was stirred at 100° C. for 16 h. The reaction mixture was concentrated under reduced pressure and purified by prep-HPLC to afford 3-(6-(((6S, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(6-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.078 g) as an off white solid.

LC-MS (ESI): m/z=827.47 [M+H]⁺

¹H NMR (400 MHz, DMSO-d6): δ 10.81 (s, 1H), 8.75 (s, 1H), 8.20 (br s, 1H), 8.03 (s, 1H), 7.85 (d, J=8.40 Hz, 1H), 7.44 (d, J=9.20 Hz, 1H), 7.28 (d, J=8.80 Hz, 1H), 6.49 (d, J=8.80 Hz, 1H), 6.35 (s, 1H), 6.15 (br s, 1H), 5.57 (br s, 1H), 4.47-4.38 (m, 2H), 4.16 (q, J=5.20 Hz, 1H), 3.79 (s, 3H), 3.68-3.63 (m, 4H), 3.57 (s, 3H), 3.21-3.18 (m, 1H), 2.98-2.95 (m, 1H), 2.59-2.51 (m, 2H), 2.25-2.13 (m, 2H), 2.00-1.86 (m, 3H), 1.62-1.53 (m, 1H), 1.46 (br s, 1H), 1.38-1.32 (m, 2H), 1.12-1.01 (m, 1H), 0.95 (d, J=6.00 Hz, 3H), 0.74-0.73 (m, 1H), 0.55-0.52 (m, 2H), 0.36-0.35 (m, 1H).

### Example 104: 3-(7-(((6R, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 465a)

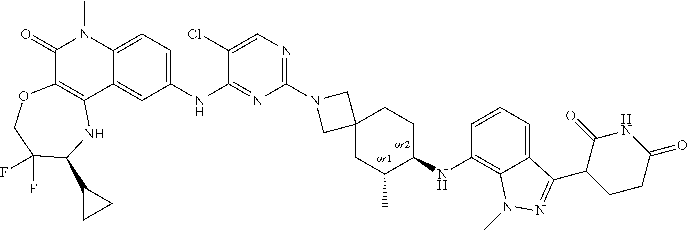 [SMILES: C[C@@H]1CC2(CC[C@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2]

### Preparation of tert-butyl (6R, 7R)-7-((3-(2,6-dioxopiperidin-3-yl)-1-methyl-1H-indazol-7-yl)amino)-6-methyl-2-azaspiro[3.5]nonane-2-carboxylate

To a solution of tert-butyl (6R, 7R)-7-((3-(2,6-bis(benzyloxy)pyridin-3-yl)-1-methyl-1H-indazol-7-yl)amino)-6-methyl-2-azaspiro[3.5]nonane-2-carboxylate (0.30 g, 0.44 mmol) in THF (12 mL) was added 20% Pd(OH)₂ on carbon (0.30 g). The reaction mixture was degassed with nitrogen.

The resulting mixture was then stirred under a H₂ atmosphere (80 psi) at room temperature for 16 h. The reaction mixture was filtered through a celite bed, washed with ethyl acetate (20 mL), concentrated under reduced pressure, washed with n-pentane and dried to afford tert-butyl (6R, 7R)-7-((3-(2,6-dioxopiperidin-3-yl)-1-methyl-1H-indazol-7-yl)amino)-6-methyl-2-azaspiro[3.5]nonane-2-carboxylate (0.2 g) as a green solid.

LC-MS (ESI): m/z=496.38 [M+H]⁺.

### Preparation of 3-(1-methyl-7-(((6R, 7R)-6-methyl-2-azaspiro[3.5]nonan-7-yl)amino)-1H-indazol-3-yl)piperidine-2,6-dione

To a stirred solution of tert-butyl (6R, 7R)-7-((3-(2,6-dioxopiperidin-3-yl)-1-methyl-1H-indazol-7-yl)amino)-6-methyl-2-azaspiro[3.5]nonane-2-carboxylate (0.2 g, 0.40 mmol)) in DCM (4.0 mL) was added TFA (2.0 mL) at 0° C. The reaction mixture was stirred at room temperature for 2 h. The reaction mixture was concentrated under reduced pressure, co-distilled with diethyl ether, and washed with n-pentane to afford 3-(1-methyl-7-(((6R, 7R)-6-methyl-2-azaspiro[3.5]nonan-7-yl)amino)-1H-indazol-3-yl)piperidine-2,6-dione (0.15 g) as a white solid.

LC-MS (ESI): m/z=396.46 [M+H]⁺.

### Preparation of 3-(7-(((6R, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a solution of 3-(1-methyl-7-(((6R, 7R)-6-methyl-2-azaspiro[3.5]nonan-7-yl)amino)-1H-indazol-3-yl)piperidine-2,6-dione (0.14 g, 0.35 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.14 mmol) in DMSO (3.0 mL) was added N,N-diisopropylethylamine (0.30 mL, 1.77 mmol). The reaction mixture was stirred at 100° C. for 16 h.

The reaction mixture was poured into ice cold water and stirred for 15 minutes. The resulting precipitate was filtered, dried, and purified by prep-HPLC to afford 3-(7-(((6R, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(7-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.011 g) as an off white solid.

LC-MS (ESI): m/z=827.47 [M+H]⁺

¹H NMR (400 MHz, DMSO-d₆): δ 10.90 (s, 1H), 8.74 (s, 1H), 8.25 (s, 1H), 8.02 (s, 1H), 7.84-7.83 (m, 1H), 7.44 (d, J=9.20 Hz, 1H), 6.96 (d, J=8.00 Hz, 1H), 6.91-6.87 (m, 1H), 6.54 (d, J=7.20 Hz, 1H), 6.11 (s, 1H), 4.44-4.42 (m, 3H), 4.26-4.24 (m, 4H), 3.62-3.57 (m, 8H), 3.23-3.22 (m, 1H), 2.62-2.61 (m, 2H), 2.33-2.32 (m, 1H), 2.20-2.15 (m, 1H), 1.69-1.63 (m, 7H), 1.40-1.30 (m, 1H), 0.98-0.96 (m, 3H), 0.72-0.71 (m, 1H), 0.51-0.53 (m, 2H), 0.37-0.36 (m, 1H).

### Example 105: 3-(6-(((6R, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 464b)

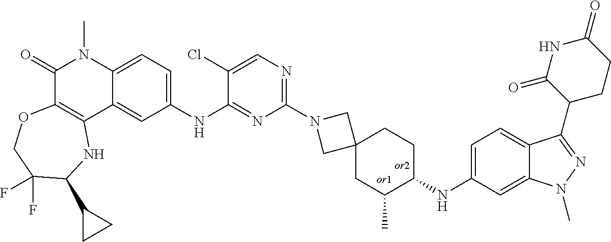 [SMILES: C[C@@H]1CC2(CC[C@@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of tert-butyl (6R, 7S)-7-((3-(2,6-dioxopiperidin-3-yl)-1-methyl-1H-indazol-6-yl)amino)-6-methyl-2-azaspiro[3.5]nonane-2-carboxylate

To a solution of tert-butyl (6R, 7S)-7-((3-(2,6-bis(benzyloxy)pyridin-3-yl)-1-methyl-1H-indazol-6-yl) amino)-6-methyl-2-azaspiro [3.5]nonane-2-carboxylate (0.52 g, 0.77 mmol) in THF (150 mL), was added 20% Pd(OH)₂ on carbon (100% w/w) (0.52 g). The reaction mixture was stirred under hydrogen atmosphere (80 psi) at room temperature for 18 h. The reaction mixture was diluted with 30% THF:DCM (900 mL) and filtered through a pad of celite. The collected filtrate was concentrated under vacuum and purified by trituration with n-pentane (30 mL) to afford tert-butyl (6R, 7S)-7-((3-(2,6-dioxopiperidin-3-yl)-1-methyl-1H-indazol-6-yl)amino)-6-methyl-2-azaspiro[3.5]nonane-2-carboxylate (0.44 g) as a pale brown semi-solid.

LC-MS (ESI): m/z=496.55 [M+H]⁺.

### Preparation of 3-(1-methyl-6-(((6R, 7S)-6-methyl-2-azaspiro[3.5]nonan-7-yl)amino)-1H-indazol-3-yl)piperidine-2,6-dione

To a solution of tert-butyl (6R, 7S)-7-((3-(2,6-dioxopiperidin-3-yl)-1-methyl-1H-indazol-6-yl)amino)-6-methyl-2-azaspiro[3.5]nonane-2-carboxylate (0.20 g, 0.40 mmol) in DCM 4.0 mL), was added TFA (2.0 mL) at 0° C. The reaction mixture was stirred at room temperature for 2 h.

The reaction mixture was concentrated under vacuum and purified by trituration with DEE (30 mL) to afford 3-(1-methyl-6-(((6R, 7S)-6-methyl-2-azaspiro[3.5]nonan-7-yl)amino)-1H-indazol-3-yl)piperidine-2,6-dione (0.24 g) as a pale brown semi-solid.

LC-MS (ESI): m/z=396.55 [M+H]⁺.

### Preparation of 3-(6-(((6R, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a stirred solution of 3-(1-methyl-6-(((6R, 7S)-6-methyl-2-azaspiro[3.5]nonan-7-yl)amino)-1H-indazol-3-yl)piperidine-2,6-dione (0.20 g, 0.50 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.09 g, 0.20 mmol) in DMSO (4.0 mL) was added DIPEA (2.0 mL). The reaction mixture was heated to 100° C. for 18 h. The reaction mixture was concentrated under reduced pressure and purified by prep-HPLC to afford 3-(6-(((6R, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(6-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.09 g) as an off white solid.

LCMS (ESI): m/z=827.47 [M+H]⁺

¹H NMR (400 MHz, DMSO-d₆): δ 10.81 (s, 1H), 8.75 (s, 1H), 8.29 (s, 1H), 1.00 (d, J=2.80 Hz, 1H), 7.86 (d, J=8.40 Hz, 1H), 7.44 (d, J=8.80 Hz, 1H), 7.30 (d, J=8.80 Hz, 1H), 6.68 (d, J=8.80 Hz, 1H), 6.39 (s, 1H), 6.12 (br s, 1H), 5.54 (d, J=8.40 Hz, 1H), 4.44-4.42 (m, 2H), 4.16-4.15 (m, 1H), 3.79 (s, 3H), 3.69 (br s, 2H), 3.62 (br s, 2H), 3.57 (s, 4H), 2.59-2.58 (m, 2H), 2.24-2.33 (m, 1H), 2.14-2.13 (m, 1H), 1.63-1.61 (m, 6H), 1.44-1.43 (m, 1H), 1.33-1.32 (m, 1H), 0.89 (d, J=6.40 Hz, 3H), 0.73 (d, J=6.80 Hz, 2H), 0.53 (d, J=6.00 Hz, 2H), 0.37-0.36 (m, 1H).

### Example 106: 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (Compound 466a)

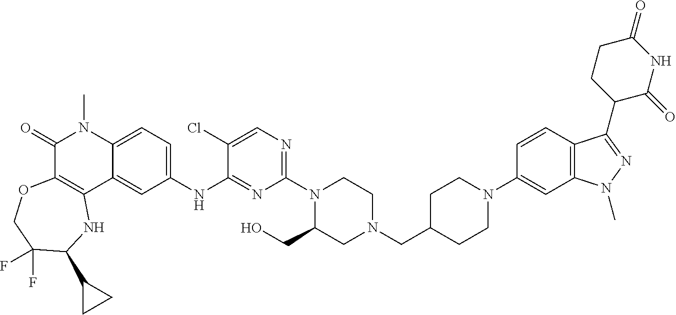 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)[C@H](CO)C4)CC3)cc21]

### Preparation of tert-butyl (S)-4-((1-((benzyloxy)carbonyl)piperidin-4-yl)methyl)-2-(hydroxymethyl)piperazine-1-carboxylate

To a solution of tert-butyl (S)-2-(hydroxymethyl)piperazine-1-carboxylate (2.0 g, 9.24 mmol), benzyl 4-formylpiperidine-1-carboxylate (2.28 g, 9.24 mmol) in DCM (40 mL) was added acetic acid (0.5 mL, 9.24 mmol). The reaction mixture was stirred at room temperature for 6 h.

NaBH(OAc)3 (2.35 g, 11.09 mmol) was then added, and the reaction was stirred at room temperature for 16 h. The reaction mixture was quenched with ice-cold water (100 mL) and extracted into DCM (3×100 mL). The combined organic layers were washed with brine (100 mL), dried over anhydrous Na₂SO₄, filtered, and dried under vacuum to get a residue, which was purified by column chromatography (SiO₂ 100-200 mesh; 40% EtOAc in petroleum ether) to afford tert-butyl (S)-4-((1-((benzyloxy)carbonyl)piperidin-4-yl)methyl)-2-(hydroxymethyl)piperazine-1-carboxylate (4.0 g) as a white solid.

LC-MS (ESI): m/z=448.32 [M+H]⁺.

### Preparation of tert-butyl-(S)-2-(hydroxymethyl)-4-(piperidin-4-ylmethyl)piperazine-1-carboxylate

To a solution of tert-butyl (S)-4-((1-((benzyloxy)carbonyl)piperidin-4-yl)methyl)-2-(hydroxymethyl)piperazine-1-carboxylate (4.0 g, 8.93 mmol) in THF (240 mL) was added 10% palladium on carbon (4.2 g, 40.21 mmol). The reaction mixture was stirred under hydrogen atmosphere (60 psi) at room temperature for 16 h. The reaction mixture was filtered through a celite bed, washed with ethyl acetate (2×100 mL), concentrated under reduced pressure, washed with n-pentane, and dried to afford tert-butyl-(S)-2-(hydroxymethyl)-4-(piperidin-4-ylmethyl)piperazine-1-carboxylate (2.5 g) as a white solid.

LC-MS (ESI): m/z=314.51 [M+H]⁺.

### Preparation of tert-butyl (S)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate

A solution of 3-(2,6-bis(benzyloxy)pyridin-3-yl)-6-bromo-1-methyl-1H-indazole (1.0 g, 1.99 mmol), tert-butyl-(S)-2-(hydroxymethyl)-4-(piperidin-4-ylmethyl)piperazine-1-carboxylate (1.5 g, 4.99 mmol) and cesium carbonate (1.9 g, 5.99 mmol) in 1,4-dioxane (20 mL) was degassed with argon for 15 minutes. Afterwards, Ruphos (0.09 g, 0.20 mmol) and RuPhos Pd G3 (0.08 g, 0.10 mmol) were added, and the reaction mixture was heated at 100° C. for 16 h. The reaction mixture was diluted with ice-cold water (100 mL) and extracted into ethyl acetate (3×100 mL).

The combined organic layers were washed with brine (100 mL), dried over anhydrous Na₂SO₄, filtered, and dried under vacuum to get a residue, which was purified by column chromatography (SiO₂ 100-200 mesh; 40% EtOAc in petroleum ether) to afford tert-butyl (S)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate (0.50 g) as a pale brown solid.

LC-MS (ESI): m/z=[M+H]⁺

### Preparation of tert-butyl-(2S)-4-((1-(3-(2,6-dioxopiperidin-3-yl)-1-methyl-1H-indazol-6-yl)piperidin-4-yl)methyl)-2-(hydroxymethyl)piperazine-1-carboxylate

To a solution of tert-butyl (S)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate (0.48 g, 0.65 mmol) in THF (36.0 mL) was added 20% Pd (OH)₂ on carbon (0.48 g, 100% w/w). The reaction mixture was stirred under hydrogen atmosphere (60 psi) at room temperature for 6 h. The reaction mixture was filtered through a celite bed, washed with ethyl acetate (100 mL), concentrated under reduced pressure, washed with n-pentane, and dried to afford tert-butyl-(2S)-4-((1-(3-(2,6-dioxopiperidin-3-yl)-1-methyl-1H-indazol-6-yl)piperidin-4-yl)methyl)-2-(hydroxymethyl)piperazine-1-carboxylate (0.34 g,) as a green solid.

LC-MS (ESI): m/z=555.67 [M+1]⁺.

### Preparation of 3-(6-(4-(((S)-3-(hydroxymethyl)piperazin-1-yl)methyl)piperidin-1-yl)-1-methyl-1H-indazol-3-yl)piperidine-2,6-dione

To a solution of tert-butyl-(2S)-4-((1-(3-(2,6-dioxopiperidin-3-yl)-1-methyl-1H-indazol-6-yl)piperidin-4-yl)methyl)-2-(hydroxymethyl)piperazine-1-carboxylate (0.38 g, 0.68 mmol) in DCM (4.0 mL) was added TFA(2.0 mL) at 0° C. The reaction mixture was stirred at room temperature for 3 h. The reaction mixture was concentrated under reduced pressure and washed with n-pentane to afford 3-(6-(4-(((S)-3-(hydroxymethyl)piperazin-1-yl)methyl)piperidin-1-yl)-1-methyl-1H-indazol-3-yl)piperidine-2,6-dione (0.25 g) as a white solid.

LC-MS (ESI): m/z=455.61 [M+H]⁺

### Preparation of 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione

To a solution 3-(6-(4-(((S)-3-(hydroxymethyl)piperazin-1-yl)methyl)piperidin-1-yl)-1-methyl-1H-indazol-3-yl)piperidine-2,6-dione (0.11 g, 0.24 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (45 mg, 0.09 mmol) in DMSO was added N,N-diisopropylethylamine(0.2 mL, 1.21 mmol). The reaction mixture was stirred at 100° C. for 16 h. The reaction mixture was poured into ice cold water (15 mL) and stirred for 15 minutes. The resulting precipitate was filtered, dried under reduced pressure and purified by prep-HPLC to afford 3-(6-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (5.0 mg) as a brown solid.

LC-MS (ESI): m/z=886.52 [M+1]⁺.

H¹ NMR (400 MHz, DMSO): δ 10.84 (s, 1H), 8.81 (s, 1H), 8.21 (s, 1H), 8.04 (s, 1H), 7.77 (s, 1H), 7.45 (dd, J=9.20, 20.40 Hz, 2H), 6.90 (d, J=10.00 Hz, 1H), 6.82 (s, 1H), 6.21 (s, 1H), 4.65 (s, 1H), 4.46-4.44 (m, 3H), 4.25-4.24 (m, 2H), 3.88 (s, 3H), 3.72-3.70 (m, 4H), 3.11-3.10 (m, 1H), 3.00-2.98 (m, 2H), 2.80-2.70 (m, 2H), 2.65-2.60 (m, 2H), 2.28-2.27 (m, 1H), 2.16-2.15 (m, 3H), 1.77-1.72 (m, 5H), 1.29-1.24 (m, 6H), 1.12-1.10 (m, 1H), 0.85-0.83 (m, 1H), 0.72-0.70 (m, 1H), 0.51-0.50 (m, 2H), 0.34-0.33 (s, 1H).

### Example 107: 3-(6-(9-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,9-diazaspiro[5.5]undecan-3-yl)-1-methyl-1H-indazol-3-yl)piperidine-2,6-dione (Compound 467a)

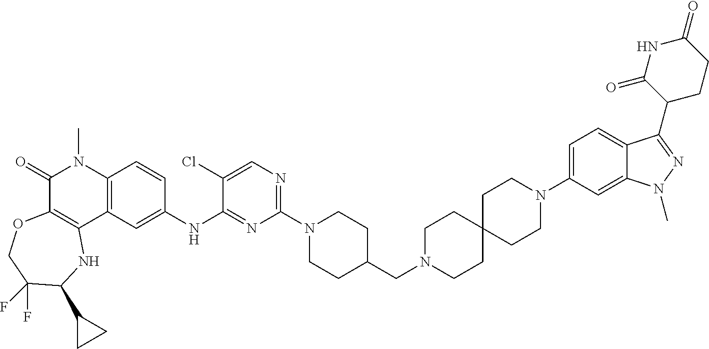 [SMILES: Cn1nc(C2CCC(=O)NC2=O)c2ccc(N3CCC4(CCN(CC5CCN(c6ncc(Cl)c(Nc7ccc8c(c7)c7c(c(=O)n8C)OCC(F)(F)[C@H](C8CC8)N7)n6)CC5)CC4)CC3)cc21]

### Preparation of tert-butyl 9-(3-(2,6-bis(benzyloxy)pyridin-3-yl)-1-methyl-1H-indazol-6-yl)-3,9-diazaspiro[5.5]undecane-3-carboxylate

To a stirred solution of 3-(2,6-bis(benzyloxy)pyridin-3-yl)-6-bromo-1-methyl-1H-indazole (3.00 g, 5.99 mmol), tert-butyl 3,9-diazaspiro [5.5]undecane-3-carboxylate (4.57 g, 17.99 mmol), and NaOtBu (1.73 g, 17.99 mmol) in 1,4-dioxane (30 mL) was added Brettphos Pd G3 (0.54 g, 0.60 mmol). The reaction mixture was degassed with argon for 5 minutes and heated to 100° C. for 18 h. The reaction mixture was diluted with EtOAc (100 mL), filtered through a celite bed, and concentrated under vacuum to obtain a residue, which was purified by column chromatography (SiO₂, 100-200 mesh; 30-50% EtOAc in petroleum ether as eluent) to afford tert-butyl 9-(3-(2,6-bis(benzyloxy)pyridin-3-yl)-1-methyl-1H-indazol-6-yl)-3,9-diazaspiro[5.5]undecane-3-carboxylate (2.02 g) as a pale yellow solid.

LC-MS (ESI): m/z=674.50 [M+H]⁺

### Preparation of 3-(3-(2,6-bis(benzyloxy)pyridin-3-yl)-1-methyl-1H-indazol-6-yl)-3,9-diazaspiro[5.5]undecane

To a solution of tert-butyl 9-(3-(2,6-bis(benzyloxy)pyridin-3-yl)-1-methyl-1H-indazol-6-yl)-3,9-diazaspiro[5.5]undecane-3-carboxylate (2.0 g, 2.97 mmol) in DCM (10 mL) was added TFA (15 mL) at 0° C. The reaction mixture was stirred at room temperature for 1 h. The reaction mixture was concentrated under reduced pressure and triturated with n-pentane (20 mL) to afford 3-(3-(2,6-bis(benzyloxy)pyridin-3-yl)-1-methyl-1H-indazol-6-yl)-3,9-diazaspiro[5.5]undecane (2.51 g) as a pale brown semi-solid.

LC-MS (ESI): m/z=574.34 [M+H]⁺.

### Preparation of benzyl 4-((9-(3-(2-(benzyloxy)-6-hydroxypyridin-3-yl)-1-methyl-1H-indazol-6-yl)-3,9-diazaspiro[5.5]undecan-3-yl)methyl)piperidine-1-carboxylate

A solution of 3-(3-(2,6-bis(benzyloxy)pyridin-3-yl)-1-methyl-1H-indazol-6-yl)-3,9-diazaspiro[5.5]undecane (2.00 g, 3.49 mmol), benzyl 4-formylpiperidine-1-carboxylate (2.50 g, 4.36 mmol), acetic acid (2.50 mL), and sodium acetate (1.07 g, 13.07 mmol) in THF (100 mL) was stirred at room temperature for 2 h. Afterwards, sodium triacetoxyborohydride (1.85 g, 8.71 mmol) was added at 0° C. The resulting mixture was stirred at room temperature for 16 h.

The reaction mixture was quenched with cold water (200 mL) and extracted with DCM (3×200 mL). The combined organic layers were washed with brine (200 mL), dried over anhydrous Na₂SO₄, filtered, and dried under vacuum to obtain a residue, which was purified by column chromatography (SiO₂; 55% EtOAc in petroleum ether as eluent) to afford benzyl 4-((9-(3-(2-(benzyloxy)-6-hydroxypyridin-3-yl)-1-methyl-1H-indazol-6-yl)-3,9-diazaspiro[5.5]undecan-3-yl)methyl)piperidine-1-carboxylate (2.01 g) as a pale brown semi-solid.

LC-MS (ESI): m/z=715.45 [M+H]⁺

### Preparation of 3-(1-methyl-6-(9-(piperidin-4-ylmethyl)-3,9-diazaspiro[5.5]undecan-3-yl)-1H-indazol-3-yl)piperidine-2,6-dione

To a solution of benzyl 4-((9-(3-(2-(benzyloxy)-6-hydroxypyridin-3-yl)-1-methyl-1H-indazol-6-yl)-3,9-diazaspiro[5.5]undecan-3-yl)methyl)piperidine-1-carboxylate (2.0 g, 2.80 mmol) in THF (80 mL) was added 20% Pd (OH)₂ on carbon (2.75 g, 19.59 mmol). The reaction mixture was stirred under hydrogen atmosphere (80 psi) at room temperature for 18 h. The reaction mixture was filtered through a celite bed and washed with THF (300 mL). The collected filtrate was concentrated under reduced pressure and washed with n-pentane (20 mL) to afford 3-(1-methyl-6-(9-(piperidin-4-ylmethyl)-3,9-diazaspiro[5.5]undecan-3-yl)-1H-indazol-3-yl)piperidine-2,6-dione (1.50 g) as a pale brown semi-solid.

LC-MS (ESI): m/z=493.69 [M+H]⁺

### Preparation of 3-(6-(9-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,9-diazaspiro[5.5]undecan-3-yl)-1-methyl-1H-indazol-3-yl)piperidine-2,6-dione

To a stirred solution of 3-(1-methyl-6-(9-(piperidin-4-ylmethyl)-3,9-diazaspiro[5.5]undecan-3-yl)-1H-indazol-3-yl)piperidine-2,6-dione (2.50 g, 5.07 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (1.19 g, 2.54 mmol) in DMSO (15 mL) was added DIPEA (15 mL). The rection mixture was heated at 100° C. for 18 h. The reaction mixture was concentrated under reduced pressure and purified by prep-HPLC to afford 3-(6-(9-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,9-diazaspiro[5.5]undecan-3-yl)-1-methyl-1H-indazol-3-yl)piperidine-2,6-dione (0.15 g) as an off white solid.

LC-MS (ESI): m/z=924.59 [M+H]⁺.

¹H NMR (400 MHz, DMSO-d₆): δ 10.85 (s, 1H), 8.88 (s, 1H), 8.17 (d, J=2.00 Hz, 1H), 8.06 (s, 1H), 7.73 (dd, J=1.60, 9.00 Hz, 1H), 7.50 (d, J=9.20 Hz, 1H), 7.42 (d, J=9.20 Hz, 1H), 6.94 (d, J=9.20 Hz, 1H), 6.86 (s, 1H), 6.24 (br s, 1H), 4.45-4.43 (m, 4H), 4.25-4.24 (m, 1H), 3.89 (s, 3H), 3.57 (s, 3H), 3.49 (br s, 7H), 3.07-3.05 (m, 4H), 2.84-2.81 (m, 2H), 2.21-2.19 (m, 1H), 2.18-2.17 (m, 2H), 1.87-1.84 (m, 2H), 1.60-1.54 (m, 8H), 1.45-1.39 (m, 1H), 1.29-1.19 (m, 1H), 1.12-1.09 (m, 2H), 0.72 (d, J=6.80 Hz, 2H), 0.52-0.51 (m, 2H), 0.34 (d, J=5.20 Hz, 1H).

### Example 108: 3-(7-(1-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)piperidin-4-yl)-1-methyl-1H-indazol-3-yl)piperidine-2,6-dione (Compound 133a)

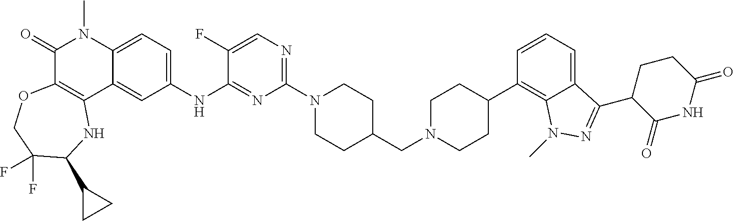 [SMILES: Cn1nc(C2CCC(=O)NC2=O)c2cccc(C3CCN(CC4CCN(c5ncc(F)c(Nc6ccc7c(c6)c6c(c(=O)n7C)OCC(F)(F)[C@H](C7CC7)N6)n5)CC4)CC3)c21]

### Preparation of (S)-10-amino-2-cyclopropyl-3,3-difluoro-7-methyl-1,2,3,4-tetrahydro-[1,4]oxazepino[2,3-c]quinolin-6(7H)-one

To a stirred solution of (S)-2-cyclopropyl-3,3-difluoro-7-methyl-10-nitro-1,2,3,4-tetrahydro-[1,4]oxazepino[2,3-c]quinolin-6(7H)-one (1.0 g, 2.84 mmol) in ethanol (40 ml) was added AcOH (0.85 mL, 14.24 mmol) and Zn powder (0.93 g, 14.24 mmol) at 0° C. The resulting mixture was stirred at room temperature for 4 h. The reaction mixture was filtered through a celite pad and washed with ethyl acetate (40 mL). The filtrate was concentrated under reduced pressure and triturated with n-pentane to afford (S)-10-amino-2-cyclopropyl-3,3-difluoro-7-methyl-1,2,3,4-tetrahydro-[1,4]oxazepino[2,3-c]quinolin-6(7H)-one (0.80 g) as a brown solid.

LC-MS (ESI): m/z=322.43 [M+H]⁺

### Preparation of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one

To a solution of (S)-10-amino-2-cyclopropyl-3,3-difluoro-7-methyl-1,2,3,4-tetrahydro-[1,4]oxazepino[2,3-c]quinolin-6(7H)-one (0.80 g, 2.49 mmol) and 2,4-dichloro-5-fluoropyrimidine (1.0 g, 6.22 mmol) in DMSO (8 mL) was added N,N-diisopropylethylamine (2.57 g, 19.92 mmol) at room temperature. The reaction mixture was stirred at 100° C. for 1 h in a microwave reactor. The reaction mixture was quenched with water and extracted with ethyl acetate. The separated organic layers were combined and washed with brine solution, dried over anhydrous Na₂SO₄, filtered, dried under vacuum and purified by flash column chromatography (SiO₂, 100-200, 60% ethyl acetate in petroleum ether) to afford (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (0.60 g) as a brown solid.

LC-MS (ESI): m/z=452.41 [M+H]⁺.

### Preparation of 3-(7-(1-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)piperidin-4-yl)-1-methyl-1H-indazol-3-yl)piperidine-2,6-dione

To a solution (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (0.35 g, 0.77 mmol) and 3-(1-methyl-7-(1-(piperidin-4-ylmethyl)piperidin-4-yl)-1H-indazol-3-yl)piperidine-2,6-dione (0.65 g, 1.55 mmol) in DMSO (7 mL) was added N,N-diisopropylethylamine (0.80 g, 6.20 mmol).

The reaction mixture was stirred at 100° C. for 16 h. The reaction mixture was concentrated under reduced pressure and purified by prep-HPLC to afford 3-(7-(1-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)piperidin-4-yl)-1-methyl-1H-indazol-3-yl)piperidine-2,6-dione (0.163 g) as a brown solid.

LC-MS (ESI): m/z=839.55 [M+H]⁺

¹H NMR (400 MHz, DMSO-d₆): δ 10.89 (s, 1H), 9.39 (s, 1H), 8.91 (s, 1H), 8.20-8.18 (m, 1H), 8.04-8.03 (d, J=3.60 Hz, 1H), 7.78-7.77 (m, 1H), 7.61-7.59 (d, J=8.00 Hz, 1H), 7.44-7.42 (d, J=8.80 Hz, 1H), 7.21-6.95 (m, 2H), 6.11 (s, 1H), 4.36-4.35 (m, 5H), 4.24 (s, 3H), 3.61-3.57 (m, 2H), 3.42 (s, 1H), 3.25-3.24 (d, J=6.40 Hz, 4H), 3.02 (m, 2H), 2.65-2.64 (m, 2H), 2.50 (m, 2H), 2.33-2.32 (d, J=1.60 Hz, 1H), 2.07-2.07-1.91 (m, 6H), 1.18-1.09 (m, 6H), 0.53-0.54 (m, 4H).

### Example 109: 3-(7-(((6R, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 465b)

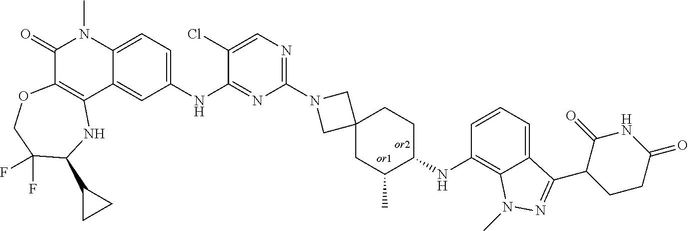 [SMILES: C[C@@H]1CC2(CC[C@@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2]

### Preparation of tert-butyl (6R, 7S)-7-((3-(2,6-bis(benzyloxy)pyridin-3-yl)-1-methyl-1H-indazol-7-yl)amino)-6-methyl-2-azaspiro[3.5]nonane-2-carboxylate

To a stirred solution of 3-(2,6-bis(benzyloxy)pyridin-3-yl)-7-bromo-1-methyl-1H-indazole (10.0 g, 19.96 mmol), tert-butyl 7-amino-6-methyl-2-azaspiro [3.5]nonane-2-carboxylate (7.61 g, 29.94 mmol) in 1,4-dioxane (100 mL) was added cesium carbonate (19.5 g, 30.0 mmol). The reaction mixture was degassed with argon for 10 minutes. Afterwards, Pd-PEPPSI-iHeptCl (0.97 g, 0.99 mmol) was added, and the reaction mixture was stirred at 100° C. for 16 h. The reaction mixture was cooled to room temperature and filtered through a celite bed. The filtrate was concentrated to get a residue, which was purified by flash column chromatography (SiO₂, 230-400, 15% ethyl acetate in petroleum ether) tert-butyl (6R, 7S)-7-((3-(2,6-bis(benzyloxy)pyridin-3-yl)-1-methyl-1H-indazol-7-yl)amino)-6-methyl-2-azaspiro[3.5]nonane-2-carboxylate (5.7 g) as an off white solid.

LC-MS (ESI): m/z=674.73 [M+H]⁺

### Preparation of tert-butyl (6R, 7S)-7-((3-(2,6-dioxopiperidin-3-yl)-1-methyl-1H-indazol-7-yl)amino)-6-methyl-2-azaspiro[3.5]nonane-2-carboxylate

To a solution of tert-butyl (6R, 7S)-7-((3-(2,6-bis(benzyloxy)pyridin-3-yl)-1-methyl-1H-indazol-7-yl)amino)-6-methyl-2-azaspiro[3.5]nonane-2-carboxylate (0.9 g, 1.33 mmol) in THF (36.0 mL) was added 20% Pd(OH)₂ on carbon (0.90 g) under nitrogen. The reaction mixture was stirred under H₂ (80 psi) at room temperature for 8 h. The reaction mixture was filtered through a celite bed, concentrated under reduced pressure, washed with n-pentane, and dried to afford tert-butyl (6R, 7S)-7-((3-(2,6-dioxopiperidin-3-yl)-1-methyl-1H-indazol-7-yl)amino)-6-methyl-2-azaspiro[3.5]nonane-2-carboxylate (0.65 g) as a green solid.

LC-MS (ESI): m/z=496.96 [M+H]⁺.

### Preparation of 3-(1-methyl-7-(((6R, 7S)-6-methyl-2-azaspiro[3.5]nonan-7-yl)amino)-1H-indazol-3-yl)piperidine-2,6-dione

To a solution of tert-butyl (6R, 7S)-7-((3-(2,6-dioxopiperidin-3-yl)-1-methyl-1H-indazol-7-yl)amino)-6-methyl-2-azaspiro[3.5]nonane-2-carboxylate (0.65 g, 1.31 mmol)) in DCM (13.0 mL) was added TFA (6.5 mL) at 0° C. The reaction mixture was stirred at room temperature for 2 h. The reaction mixture was concentrated under reduced pressure and washed with n-pentane to afford 3-(1-methyl-7-(((6R, 7S)-6-methyl-2-azaspiro[3.5]nonan-7-yl)amino)-1H-indazol-3-yl)piperidine-2,6-dione (0.6 g) as a brown solid.

LC-MS (ESI): m/z=396.46 [M+H]⁺.

### Preparation of 3-(7-(((6R, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a solution of 3-(1-methyl-7-(((6R, 7S)-6-methyl-2-azaspiro[3.5]nonan-7-yl)amino)-1H-indazol-3-yl)piperidine-2,6-dione (0.3 g, 0.76 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.21 g, 0.45 mmol) in DMSO (6.0 mL) was added N,N-diisopropylethylamine (0.78 g, 6.08 mmol). The reaction mixture stirred at 100° C. for 5 h. The reaction mixture was poured into ice cold water and stirred for 15 minutes. The resulting precipitate was filtered, dried under reduced pressure, and purified by prep-HPLC to afford 3-(7-(((6R, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(7-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.10 g) as an off white solid.

LC-MS (ESI): m/z=827.51 [M+H]⁺

¹H NMR (400 MHz, DMSO-d6): δ 10.85 (s, 1H), 9.30 (s, 1H), 8.18 (d, J=14.40 Hz, 2H), 7.77 (s, 1H), 7.46 (d, J=9.20 Hz, 1H), 6.86-6.84 (m, 2H), 6.46 (d, J=6.80 Hz, 1H), 6.23 (s, 1H), 4.75 (s, 1H), 4.44-4.42 (m, 2H), 4.25-4.22 (m, 4H), 3.69-3.57 (m, 7H), 3.25 (s, 1H), 2.95 (s, 1H), 2.61-2.59 (m, 2H), 2.29-2.26 (m, 1H), 2.14-2.12 (m, 5H), 1.64-1.61 (m, 2H), 1.34-1.33 (m, 2H), 1.24-1.22 (m, 1H), 1.00 (d, J=6.00 Hz, 3H), 0.73 (d, J=6.80 Hz, 1H), 0.53 (t, J=6.00 Hz, 2H).

### Example 110: 3-(6-(((6S, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 464c)

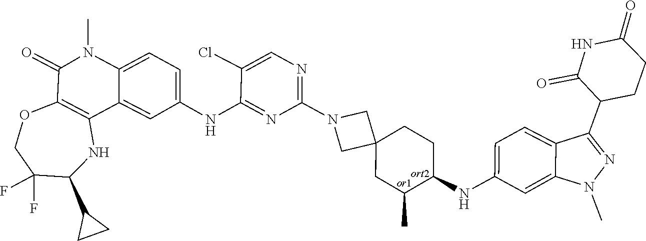 [SMILES: C[C@H]1CC2(CC[C@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of tert-butyl 6-methyl-7-oxo-2-azaspiro[3.5]nonane-2-carboxylate

To a suspension of tert-butyl 7-oxo-2-azaspiro[3.5]nonane-2-carboxylate (90.0 g, 0.38 mol) in THF (50 mL) was added LDA (0.9 L) at 0° C. The reaction mixture was stirred for 1 h. Iodomethane (70.0 mL, 1.13 mol) was then added slowly over a period of 30 min at 0° C., and the resulting mixture was stirred at room temperature for 6 h. The reaction mixture was diluted with ice water (2 L) and extracted with ethyl acetate (2×1 L). The separated organic layer was washed with brine, dried over anhydrous Na₂SO₄, filtered, and dried under vacuum to get a residue which was purified by column chromatography (SiO₂, 100-200 mesh, 15% EtOAc in petroleum ether) to afford tert-butyl 6-methyl-7-oxo-2-azaspiro[3.5]nonane-2-carboxylate (35 g) as a yellow solid.

¹H NMR (400 MHz, DMSO-d6): δ 3.89-3.87 (m, 2H), 3.65 (s, 2H), 2.34-2.33 (m, 2H), 2.22-2.21 (m, 2H), 1.86-1.85 (m, 1H), 1.60-1.58 (m, 2H), 1.46 (s, 9H), 1.03 (d, J=6.80 Hz, 3H).

### Preparation of tert-butyl 7-amino-6-methyl-2-azaspiro[3.5]nonane-2-carboxylate

To a suspension of tert-butyl 6-methyl-7-oxo-2-azaspiro[3.5]nonane-2-carboxylate (35.0 g, 138.3 mol) in methanol (350 mL) was added NH₃ in methanol (0.35 L, 7.0 M) at room temperature.

Sodium borohydride (10.46 g, 276.7 mmol) was slowly added to the reaction. The reaction mixture was stirred for 8 h. The reaction mixture was concentrated under reduced pressure, diluted with ice water (1 L), and extracted with ethyl acetate (2×0.5 L). The separated organic layer was washed with brine, dried over anhydrous Na₂SO₄, filtered, dried under vacuum, and triturated using diethyl ether to afford tert-butyl 7-amino-6-methyl-2-azaspiro[3.5]nonane-2-carboxylate (35.0 g) as a yellow semi-solid.

LC-MS (ESI): m/z=255.28 [M+H]⁺

### Preparation of tert-butyl (6S, 7R)-7-((3-(2,6-bis(benzyloxy)pyridin-3-yl)-1-methyl-1H-indazol-6-yl)amino)-6-methyl-2-azaspiro[3.5]nonane-2-carboxylate

To a solution of 3-(2,6-bis(benzyloxy)pyridin-3-yl)-6-bromo-1-methyl-1H-indazole (5.0 g, 10.0 mmol) and tert-butyl 7-amino-6-methyl-2-azaspiro[3.5]nonane-2-carboxylate (10.1 g, 40.0 mmol) in 1,4-dioxane (100 mL) was added cesium carbonate (9.76 g, 30.0 mmol) under argon, followed by the addition of RuPhos (0.47 g, 1.0 mmol) and RuPhos-PdG3 (0.04 g, 0.05 mmol). The reaction mixture was stirred at 80° C. for 16 h under argon. The reaction mixture was cooled to room temperature and filtered through a celite bed. The filtrate was concentrated and purified by flash column chromatography (SiO₂, 230-400, 30% ethyl acetate in petroleum ether) to afford tert-butyl 7-((3-(2,6-bis(benzyloxy)pyridin-3-yl)-1-methyl-1H-indazol-6-yl)amino)-6-methyl-2-azaspiro[3.5]nonane-2-carboxylate (3.5 g) as an off white solid. Diastereomeric mixture was subjected to SFC separation to isolate four isomers, Isomer 1 was assigned arbitrarily as tert-butyl (6S, 7R)-7-((3-(2,6-bis(benzyloxy)pyridin-3-yl)-1-methyl-1H-indazol-6-yl)amino)-6-methyl-2-azaspiro[3.5]nonane-2-carboxylate (0.60 g) for ease of exposition.

LC-MS (ESI): m/z=674.43 [M+H]⁺

### Preparation of tert-butyl (6S, 7R)-7-((3-(2,6-dioxopiperidin-3-yl)-1-methyl-1H-indazol-6-yl)amino)-6-methyl-2-azaspiro[3.5]nonane-2-carboxylate

A stirred solution of tert-butyl (6S, 7R)-7-((3-(2,6-bis(benzyloxy)pyridin-3-yl)-1-methyl-1H-indazol-6-yl)amino)-6-methyl-2-azaspiro[3.5]nonane-2-carboxylate (0.60 g, 0.90 mmol) in THF (25 mL) was degassed with nitrogen, followed by the addition of 20% Pd(OH)₂ on carbon (0.60 g). The resulting mixture was stirred under H₂ (80 psi) at room temperature for 16 h. The reaction mixture was filtered through a celite bed, concentrated, washed with n-pentane, and dried to afford tert-butyl (6S, 7R)-7-((3-(2,6-dioxopiperidin-3-yl)-1-methyl-1H-indazol-6-yl)amino)-6-methyl-2-azaspiro[3.5]nonane-2-carboxylate (0.45 g) as a green solid.

LC-MS (ESI): m/z=496.40 [M+H]⁺

### Preparation of 3-(1-methyl-6-(((6S, 7R)-6-methyl-2-azaspiro[3.5]nonan-7-yl)amino)-1H-indazol-3-yl)piperidine-2,6-dione

To a stirred solution of tert-butyl (6S, 7R)-7-((3-(2,6-dioxopiperidin-3-yl)-1-methyl-1H-indazol-6-yl)amino)-6-methyl-2-azaspiro[3.5]nonane-2-carboxylate (0.45 g, 0.90 mmol)) in DCM (4.5 mL), was added TFA (4.5 mL) at 0° C. The reaction mixture was stirred at room temperature for 2 h.

The reaction mixture was concentrated under reduced pressure and washed with n-pentane to afford 3-(1-methyl-6-(((6S, 7R)-6-methyl-2-azaspiro[3.5]nonan-7-yl)amino)-1H-indazol-3-yl)piperidine-2,6-dione (0.3 g) as a brown solid.

LC-MS (ESI): m/z=395.63 [M+H]⁺.

### Preparation of 3-(6-(((6S, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a solution of 3-(1-methyl-6-(((6S, 7R)-6-methyl-2-azaspiro[3.5]nonan-7-yl)amino)-1H-indazol-3-yl)piperidine-2,6-dione (0.15 g, 0.38 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.1 g, 0.23 mmol) in DMSO (3 mL) was added N, N-diisopropylethylamine (0.39 g, 2.72 mmol). The reaction mixture was stirred at 100° C. for 5 h. The reaction mixture was poured into ice cold water and stirred for 15 minutes. The resulting precipitate was filtered, dried under reduced pressure and purified by prep-HPLC to afford 3-(6-(((6S, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(6-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.080 g) as an off white solid.

LC-MS (ESI): m/z=827.51 [M+H]⁺

¹H NMR (400 MHz, DMSO-d6): 10.81 (s, 1H), 8.22 (d, J=22.80 Hz, 1H), 8.03 (s, 1H), 7.84 (dd, J=2.00, 9.00 Hz, 1H), 7.44 (d, J=9.20 Hz, 1H), 7.28 (d, J=8.80 Hz, 1H), 6.49 (d, J=8.80 Hz, 1H), 6.35 (s, 1H), 6.15 (s, 1H), 5.56 (s, 1H), 4.42-4.38 (m, 2H), 4.15-4.14 (m, 1H), 3.79 (s, 3H), 3.60-3.57 (m, 7H), 3.22 (s, 1H), 2.59-2.58 (m, 1H), 2.51-2.50 (m, 2H), 2.25-2.23 (m, 2H), 2.14-2.12 (m, 2H), 1.91-1.86 (m, 3H), 1.60-1.59 (m, 2H), 1.34-1.32 (m, 2H), 0.96-0.94 (m, 3H), 0.71-0.70 (m, 1H), 0.54-0.52 (m, 2H), 0.37-0.35 (m, 1H).

### Example 111: 3-(7-(((6S, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 465c)

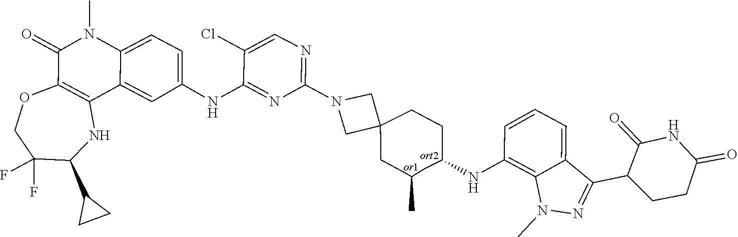

### Preparation of tert-butyl-(6S, 7S)-7-((3-(2,6-dioxopiperidin-3-yl)-1-methyl-1H-indazol-7-yl)amino)-6-methyl-2-azaspiro[3.5]nonane-2-carboxylate

To a solution of tert-butyl (6S, 7S)-7-((3-(2,6-bis(benzyloxy)pyridin-3-yl)-1-methyl-1H-indazol-7-yl) amino)-6-methyl-2-azaspiro [3.5]nonane-2-carboxylate (0.22 g, 0.33 mmol) in THF (88.0 mL), 20% Pd (OH)₂/C (0.22 g, 100% w/w) was added. The reaction mixture was stirred under hydrogen atmosphere (80 psi) at room temperature for 18 h. The reaction mixture was filtered through a celite bed and washed with excess of 30% THF in DCM (500 mL). The collected filtrate was concentrated under vacuum, washed with n-pentane (10 mL), and dried to afford tert-butyl-(6S, 7S)-7-((3-(2,6-dioxopiperidin-3-yl)-1-methyl-1H-indazol-7-yl)amino)-6-methyl-2-azaspiro[3.5]nonane-2-carboxylate (0.19 g) as a pale brown semi-solid.

LCMS (ESI): m/z=496.55 [M+H]⁺

### Preparation of 3-(1-methyl-7-(((6S, 7S)-6-methyl-2-azaspiro[3.5]nonan-7-yl)amino)-1H-indazol-3-yl)piperidine-2,6-dione

To a solution of tert-butyl-(6S, 7S)-7-((3-(2,6-dioxopiperidin-3-yl)-1-methyl-1H-indazol-7-yl)amino)-6-methyl-2-azaspiro[3.5]nonane-2-carboxylate (0.19 g, 0.38 mmol) in DCM (5.13 mL) was added TFA (1.42 mL) at 0° C. The reaction was stirred at room temperature for 1 h. The reaction mixture was concentrated under reduced pressure and triturated with n-pentane (20 mL) to afford 3-(1-methyl-7-(((6S, 7S)-6-methyl-2-azaspiro[3.5]nonan-7-yl)amino)-1H-indazol-3-yl)piperidine-2,6-dione (0.20 g) as a pale brown semi solid.

LC-MS (ESI): m/z=396.50 [M+H]⁺

### Preparation of 3-(7-(((6S, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a stirred solution of 3-(1-methyl-7-(((6S, 7S)-6-methyl-2-azaspiro[3.5]nonan-7-yl)amino)-1H-indazol-3-yl)piperidine-2,6-dione (0.20 g, 0.51 mmol) in DMSO (2 mL) was added (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.12 g, 0.25 mmol) and DIPEA (2 mL). The resulting mixture was heated at 100° C. for 18 h. The reaction mixture was concentrated under reduced pressure and purified by prep-HPLC to afford 3-(7-(((6S, 7S)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(7-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.017 g) as an off white solid.

LC-MS (ESI): 727.43 [M+H]⁺.

¹H NMR (400 MHz, DMSO-d₆): δ 10.85 (s, 1H), 8.76 (s, 1H), 8.53 (s, 1H), 8.25 (s, 1H), 8.02 (s, 1H), 7.84 (d, J=9.20 Hz, 1H), 7.43 (d, J=9.20 Hz, 1H), 6.96 (d, J=8.00 Hz, 1H), 6.89-6.87 (m, 1H), 6.54 (d, J=7.20 Hz, 1H), 6.16 (s, 1H), 4.45-4.43 (m, 3H), 4.26-4.26 (m, 4H), 3.74-3.72 (m, 6H), 3.66 (d, J=10.80 Hz, 3H), 3.32-3.24 (m, 1H), 2.67-2.67 (m, 2H), 2.35-2.32 (m, 1H), 2.23-2.21 (m, 1H), 1.94-1.93 (m, 5H), 0.97 (d, J=6.80 Hz, 3H), 0.68-0.66 (m, 2H), 0.52-0.51 (m, 2H), 0.34-0.32 (m, 1H).

### Example 112: 3-(7-(((6S, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 465d)

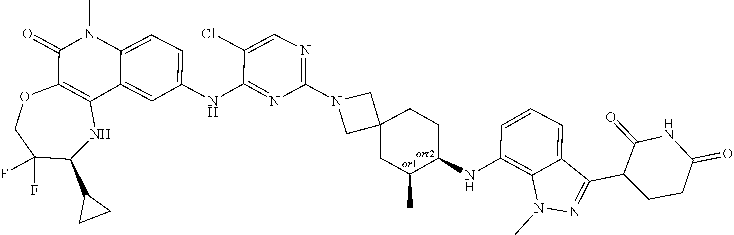 [SMILES: C[C@H]1CC2(CC[C@H]1Nc1cccc3c(C4CCC(=O)NC4=O)nn(C)c13)CN(c1ncc(Cl)c(Nc3ccc4c(c3)c3c(c(=O)n4C)OCC(F)(F)[C@H](C4CC4)N3)n1)C2]

### Preparation of tert-butyl (6S, 7R)-7-((3-(2,6-dioxopiperidin-3-yl)-1-methyl-1H-indazol-7-yl)amino)-6-methyl-2-azaspiro[3.5]nonane-2-carboxylate

To a solution of tert-butyl (6S, 7R)-7-((3-(2,6-bis(benzyloxy)pyridin-3-yl)-1-methyl-1H-indazol-7-yl)amino)-6-methyl-2-azaspiro[3.5]nonane-2-carboxylate (0.60 g, 0.89 mmol) in THF (30.0 mL) was added 20% Pd(OH)₂ on carbon (0.60 g) and stirred under H₂ (60 psi) atmosphere at room temperature for 8 h. The reaction mixture was filtered through a celite bed and washed with THF:DCM (1:1) (500 mL). The collected filtrate was concentrated under reduced pressure to afford tert-butyl (6S, 7R)-7-((3-(2,6-dioxopiperidin-3-yl)-1-methyl-1H-indazol-7-yl)amino)-6-methyl-2-azaspiro[3.5]nonane-2-carboxylate (0.45 g) as brown solid which was used in the next step without purification.

LC-MS (ESI): m/z=496.5 [M+H]⁺.

### Preparation of 3-(1-methyl-7-(((6S, 7R)-6-methyl-2-azaspiro[3.5]nonan-7-yl)amino)-1H-indazol-3-yl)piperidine-2,6-dione

To a solution of tert-butyl (6S, 7R)-7-((3-(2,6-dioxopiperidin-3-yl)-1-methyl-1H-indazol-7-yl)amino)-6-methyl-2-azaspiro[3.5]nonane-2-carboxylate (0.45 g, 0.90 mmol) in DCM (9.0 mL) was added TFA (0.45 mL) at 0° C. The reaction mixture was stirred at room temperature for 3 h.

The reaction mixture was concentrated, co-distilled with DCM (3×40 mL), dried under reduced pressure, and triturated with diethyl ether (30 mL) to afford 3-(1-methyl-7-(((6S, 7R)-6-methyl-2-azaspiro[3.5]nonan-7-yl)amino)-1H-indazol-3-yl)piperidine-2,6-dione (0.6 g) as a brown solid.

LC-MS (ESI): m/z=396.46 [M+H]⁺.

### Preparation of 3-(7-(((6S, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(7-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a solution of 3-(1-methyl-7-(((6S, 7R)-6-methyl-2-azaspiro[3.5]nonan-7-yl)amino)-1H-indazol-3-yl)piperidine-2,6-dione (0.30 g, 0.76 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.14 g, 0.30 mmol) in DMSO (3.0 mL) was added N,N-diisopropylethylamine (0.6 mL, 3.80 mmol) and stirred at 100° C. for 2 h. The reaction mixture was concentrated under reduced pressure and purified by prep-HPLC to afford 3-(7-(((6S, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(7-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.042 g) as an off white solid.

LC-MS (ESI): m/z=827.5 [M+H]⁺.

¹H NMR (400 MHz, DMSO-d₆): 10.84 (s, 1H), 8.74 (s, 1H), 8.03 (s, 1H), 7.82 (d, J=7.60 Hz, 1H), 7.43 (d, J=9.20 Hz, 1H), 6.90-6.84 (m, 2H), 6.47 (d, J=6.80 Hz, 1H), 6.12 (br, s, 1H), δ 4.80-4.60 (m, 1H), 4.42-4.26 (m, 2H), 4.25-4.23 (m, 4H), 3.70-3.68 (m, 5H), 3.60 (s, 3H), 3.31-3.326 (m, 1H), 2.90-2.99 (m, 1H), 2.61-2.59 (m, 2H), 2.50-2.14 (m, 1H), 2.14-1.97 (m, 1H), 1.91-1.90 (m, 2H), 1.67-1.66 (m, 2H), 1.36-1.33 (m, 4H), 1.00 (d, J=6.00 Hz, 3H), 0.54-0.52 (m, 1H), 0.51-0.41 (m, 2H), 0.39-0.29 (m, 1H).

### Example 113: 3-(6-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)-1-methyl-1H-indazol-3-yl)piperidine-2,6-dione (Compound 468a)

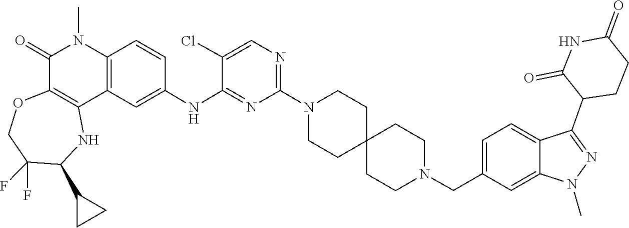 [SMILES: Cn1nc(C2CCC(=O)NC2=O)c2ccc(CN3CCC4(CC3)CCN(c3ncc(Cl)c(Nc5ccc6c(c5)c5c(c(=O)n6C)OCC(F)(F)[C@H](C6CC6)N5)n3)CC4)cc21]

### Preparation of 3-(2,6-bis(benzyloxy)pyridin-3-yl)-1-methyl-1H-indazole-6-carbaldehyde

To a stirred solution of 3-(2,6-bis(benzyloxy)pyridin-3-yl)-6-bromo-1-methyl-1H-indazole (5.0 g, 10.0 mmol) in THF (100 mL) at −78° C. was added a solution of N-methylmorpholine (1.0 mL, 8.00 mmol) and n-BuLi (2.5M) (4.6 mL) in DMF dropwise. The reaction mixture was quenched with 1M aqueous ammonium chloride solution which was added dropwise (20 mL) until the reaction mixture was warmed to room temperature. The reaction mixture was extracted with tert-butyl methyl ether (3×50 mL). The organic layer was separated, washed with brine (50 mL), dried over anhydrous Na₂SO₄, filtered, and concentrated under vacuum to get a residue which was purified by silica column chromatography (SiO₂; 40% EtOAc in petroleum ether) to afford 3-(2,6-bis(benzyloxy)pyridin-3-yl)-1-methyl-1H-indazole-6-carbaldehyde, (1.7 g) as a light yellow solid.

LC-MS (ESI): m/z=450.24 [M+H]⁺

### Preparation of tert-butyl 9-((3-(2,6-bis(benzyloxy)pyridin-3-yl)-1-methyl-1H-indazol-6-yl)methyl)-3,9-diazaspiro[5.5]undecane-3-carboxylate

To a solution of 3-(2,6-bis(benzyloxy)pyridin-3-yl)-1-methyl-1H-indazole-6-carbaldehyde (1.5 g, 3.33 mmol) and tert-butyl 3,9-diazaspiro[5.5]undecane-3-carboxylate (1.27 g 5.005 mmol) in THF (15.0 mL), was added Ti(OiPr)₄ (7.5 mL) at room temperature. The reaction mixture was stirred for 1 h. Sodium cyanoborohydride (0.62 g, 10.0 mmol) was then added at 0° C., and the resulting mixture was stirred at room temperature for 3 h. The reaction mixture was quenched with water (30.0 mL) and extracted with DCM (100.0 mL). The organic layer was separated, washed with brine (30.0 mL), dried over anhydrous Na₂SO₄, filtered, and dried under vacuum to get a residue which was purified by flash column chromatography (SiO₂, 230-400 mesh; 80% EtOAc in petroleum ether) to afford tert-butyl 9-((3-(2,6-bis(benzyloxy)pyridin-3-yl)-1-methyl-1H-indazol-6-yl)methyl)-3,9-diazaspiro[5.5]undecane-3-carboxylate (0.7 g) as an off white solid.

LC-MS (ESI): m/z=688.75 [M+H]⁺

### Preparation of tert-butyl 9-((3-(2,6-dioxopiperidin-3-yl)-1-methyl-1H-indazol-6-yl)methyl)-3,9-diazaspiro[5.5]undecane-3-carboxylate

A stirred solution of tert-butyl 9-((3-(2,6-bis(benzyloxy)pyridin-3-yl)-1-methyl-1H-indazol-6-yl)methyl)-3,9-diazaspiro[5.5]undecane-3-carboxylate (0.3 g, 0.41 mmol) in THF (15.0 mL) at room temperature was purged with N₂. Acetic acid (0.01 mL), DMF (0.01 mL) and Pd(OH)₂ (0.58 g, 4.18 mmol) were then added, and the reaction mixture was stirred under hydrogen atmosphere (30 psi) for 8 h. The reaction mixture was filtered through a celite pad and washed with EtOAc (3×50 mL). The filtrate was concentrated under reduced pressure to get a residue which was purified by flash column chromatography (SiO₂, 230-400 mesh; 100% EtOAc) to afford tert-butyl 9-((3-(2,6-dioxopiperidin-3-yl)-1-methyl-1H-indazol-6-yl)methyl)-3,9-diazaspiro[5.5]undecane-3-carboxylate, (0.08 g) as an off white solid.

LC-MS (ESI): m/z=510.36 [M+H]⁺

### Preparation of 3-(6-((3,9-diazaspiro[5.5]undecan-3-yl)methyl)-1-methyl-1H-indazol-3-yl)piperidine-2,6-dione

To a solution of tert-butyl 9-((3-(2,6-dioxopiperidin-3-yl)-1-methyl-1H-indazol-6-yl)methyl)-3,9-diazaspiro[5.5]undecane-3-carboxylate (0.08 g, 0.157 mmol) in DCM (1.2 mL) at 0° C. was added TFA (0.4 mL). The reaction mixture was stirred at room temperature for 2 h. The reaction mixture was concentrated under reduced pressure and triturated with n-pentane (10 mL) to afford 3-(6-((3,9-diazaspiro[5.5]undecan-3-yl)methyl)-1-methyl-1H-indazol-3-yl)piperidine-2,6-dione (0.1 g) as a liquid.

LC-MS (ESI): m/z=410.57 [M+H]⁺.

### Preparation of 3-(6-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)-1-methyl-1H-indazol-3-yl)piperidine-2,6-dione

To a solution of 3-(6-((3,9-diazaspiro[5.5]undecan-3-yl)methyl)-1-methyl-1H-indazol-3-yl)piperidine-2,6-dione (0.08 g, 0.195 mmol) in DMSO (2.4 mL) was added N,N-diisopropylethylamine (0.34 mL) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.046 g, 0.098 mmol). The reaction mixture was stirred at 100° C. for 6 h. The reaction mixture was concentrated under reduced pressure and purified by prep-HPLC to afford 3-(6-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)-1-methyl-1H-indazol-3-yl)piperidine-2,6-dione (0.017 g) as an off white solid.

LC-MS (ESI): m/z=841.60 [M+H]⁺

¹HNMR: 400 MHz, DMSO-d₆: δ 10.88 (s, 1H), 8.77 (s, 1H), 8.41 (s, 1H), 8.19 (d, J=1.6 Hz, 1H), 8.01 (s, 1H), 7.72 (d, J=10.2 Hz, 1H), 7.63 (d, J=8.0 Hz, 1H), 7.44-7.42 (m, 2H), 7.08 (d, J=8.0 Hz, 1H), 6.19 (s, 1H), 4.44-4.32 (m, 3H), 3.96 (s, 3H), 3.56 (s, 9H), 2.67-2.61 (m, 2H), 2.50-2.49 (m, 5H), 2.36-2.32 (m, 1H), 1.45-1.23 (m, 9H), 0.70 (s, 1H), 0.51-0.50 (m, 2H), 0.34 (m, 1H).

### Example 114: 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)piperidin-1-yl)-7-fluoro-1-methyl-1H-indazol-3-yl)piperidine-2,6-dione (Compound 469a)

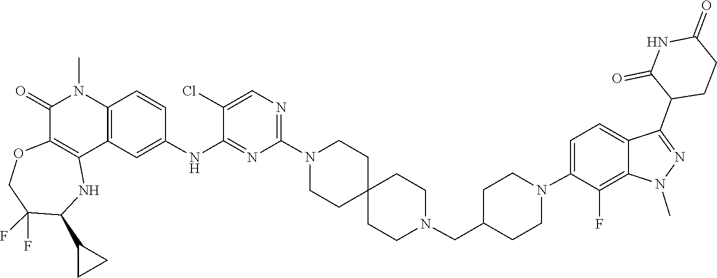 [SMILES: Cn1nc(C2CCC(=O)NC2=O)c2ccc(N3CCC(CN4CCC5(CC4)CCN(c4ncc(Cl)c(Nc6ccc7c(c6)c6c(c(=O)n7C)OCC(F)(F)[C@H](C7CC7)N6)n4)CC5)CC3)c(F)c21]

### Preparation of tert-butyl-9-(piperidin-4-ylmethyl)-3,9-diazaspiro[5.5]undecane-3-carboxylate

To a stirred solution of tert-butyl 9-((1-((benzyloxy)carbonyl) piperidin-4-yl) methyl)-3,9-diazaspiro [5.5]undecane-3-carboxylate (5.0 g, 2.06 mmol) in tetrahydrofuran (50 ml) was added palladium carbon (5.0 g) under inert atmosphere at room temperature. The reaction mixture was stirred under hydrogen pressure (50 Psi) at room temperature for 16 h. After completion, the reaction mixture was filtered through a celite bed, and the filtrate was concentrated under reduced pressure to provide tert-butyl-9-(piperidin-4-ylmethyl)-3,9-diazaspiro[5.5]undecane-3-carboxylate (2.5 g) as a brown solid which was used in the next step without further purification.

LC-MS (ESI): m/z=352.68 [M+H]⁺.

### Preparation of tert-butyl 9-((1-(3-(2,6-bis(benzyloxy)pyridin-3-yl)-7-fluoro-1-methyl-1H-indazol-6-yl)piperidin-4-yl)methyl)-3,9-diazaspiro[5.5]undecane-3-carboxylate

A stirred solution of tert-butyl-9-(piperidin-4-ylmethyl)-3,9-diazaspiro[5.5]undecane-3-carboxylate (0.75 g, 2.13 mmol), 3-(2,6-bis(benzyloxy)pyridin-3-yl)-6-bromo-7-fluoro-1-methyl-1H-indazole (1.10 g, 2.13 mmol), and cesium carbonate (2.08 g, 6.39 mmol) in 1,4-dioxane (10 m1) was degassed with nitrogen for 5 min. Pd-PEPPSI-IHeptCl (0.20 g, 0.21 mmol) was then added, and the resulting mixture was stirred at 110° C. for 12 h. The reaction mixture was filtered through a celite bed, and the filtrate was concentrated under reduced pressure to give a residue which was purified by flash column chromatography (SiO₂, 100-200 mesh, 60% ethyl acetate in petroleum ether) to afford tert-butyl 9-((1-(3-(2,6-bis(benzyloxy)pyridin-3-yl)-7-fluoro-1-methyl-1H-indazol-6-yl)piperidin-4-yl)methyl)-3,9-diazaspiro[5.5]undecane-3-carboxylate (0.35 g) as a brown solid.

LC-MS (ESI): m/z=789.51 [M+H]⁺.

### Preparation of tert-butyl 9-((1-(3-(2,6-dioxopiperidin-3-yl)-7-fluoro-1-methyl-1H-indazol-6-yl)piperidin-4-yl)methyl)-3,9-diazaspiro[5.5]undecane-3-carboxylate

To a stirred suspension of tert-butyl 9-((1-(3-(2,6-bis(benzyloxy)pyridin-3-yl)-7-fluoro-1-methyl-1H-indazol-6-yl)piperidin-4-yl)methyl)-3,9-diazaspiro[5.5]undecane-3-carboxylate (0.35 g, 0.44 mmol) in THF (10 mL) was added 20% Pd(OH)₂ on carbon (100% w/w). The reaction mixture was hydrogenated using a par shaker (80 psi) at room temperature for 7 h. After completion, the reaction mixture was filtered through a celite bed and washed with THF. The filtrate was concentrated under reduced pressure to afford tert-butyl 9-((1-(3-(2,6-dioxopiperidin-3-yl)-7-fluoro-1-methyl-1H-indazol-6-yl)piperidin-4-yl)methyl)-3,9-diazaspiro[5.5]undecane-3-carboxylate (0.24 g) as an off-white solid which was used in the next step without purification.

LC-MS (ESI): m/z=611.42 [M+H]⁺

### Preparation of 3-(6-(4-((3,9-diazaspiro[5.5]undecan-3-yl)methyl)piperidin-1-yl)-7-fluoro-1-methyl-1H-indazol-3-yl)piperidine-2,6-dione

To a stirred suspension of tert-butyl 9-((1-(3-(2,6-dioxopiperidin-3-yl)-7-fluoro-1-methyl-1H-indazol-6-yl)piperidin-4-yl)methyl)-3,9-diazaspiro[5.5]undecane-3-carboxylate (0.25 g, 0.40 mmol) in DCM (2.5 mL) was added TFA (1.25 mL) at 0° C. The reaction mixture was stirred at room temperature for 3 h. After completion of reaction, the reaction mixture was concentrated under reduced pressure and purified by trituration with pentane (10 mL) to afford 3-(6-(4-((3,9-diazaspiro[5.5]undecan-3-yl)methyl)piperidin-1-yl)-7-fluoro-1-methyl-1H-indazol-3-yl)piperidine-2,6-dione (0.2 g) as a brown gum.

LC-MS (ESI): m/z=511.70 [M+H]⁺

### Preparation of 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)piperidin-1-yl)-7-fluoro-1-methyl-1H-indazol-3-yl)piperidine-2,6-dione

To a solution of 3-(6-(4-((3,9-diazaspiro[5.5]undecan-3-yl)methyl)piperidin-1-yl)-7-fluoro-1-methyl-1H-indazol-3-yl)piperidine-2,6-dione (0.20 g, 0.39 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.09 g, 0.19 mmol) in DMSO (4 mL) was added N,N-diisopropylethylamine (0.25 ml, 1.95 mmol) at room temperature. The resulting mixture was stirred at 110° C. for 4 h. After completion of reaction, the reaction mixture was concentrated under reduced pressure and subjected to prep-HPLC purification to afford 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,9-diazaspiro[5.5]undecan-3-yl)methyl)piperidin-1-yl)-7-fluoro-1-methyl-1H-indazol-3-yl)piperidine-2,6-dione (0.028 g) as a light green solid.

LC-MS (ESI): m/z=942.60 [M+H]⁺

¹H NMR (400 MHz, DMSO-d₆): δ 10.90 (s, 1H), 8.77 (s, 1H), 8.28 (br s, 1H), 8.19 (s, 1H), 8.02 (s, 1H), 7.71 (d, J=1.60 Hz, 1H), 7.44 (d, J=9.20 Hz, 1H), 7.38 (d, J=8.40 Hz, 1H), 6.92 (t, J=8.00 Hz, 1H), 6.19 (br s, 1H), 4.31-4.29 (m, 3H), 4.06 (s, 3H), 3.70-3.40 (m, 6H), 3.30-3.20 (m, 2H), 2.61-2.59 (m, 4H), 2.32 (bs, 5H), 2.14-2.06 (m, 3H), 1.80-1.75 (m, 2H), 1.62-1.57 (m, 1H), 1.45-1.39 (m, 4H), 1.31-1.29 (m, 7H), 0.85-0.73 (m, 1H), 0.60-0.55 (m, 2H), 0.40-0.28 (m, 1H).

### Example 115: 3-(6-(4-(2-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)propan-2-yl)piperazin-1-yl)-1-methyl-1H-indazol-3-yl)piperidine-2,6-dione (Compound 470a)

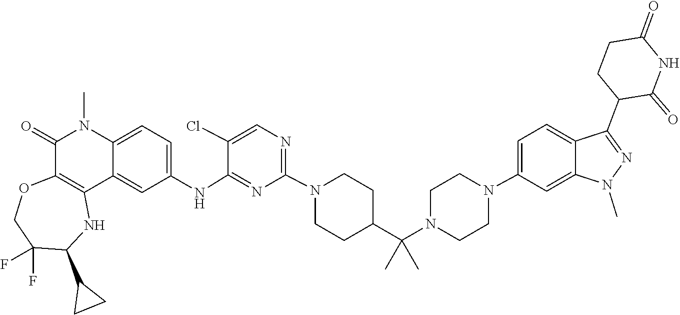 [SMILES: Cn1nc(C2CCC(=O)NC2=O)c2ccc(N3CCN(C(C)(C)C4CCN(c5ncc(Cl)c(Nc6ccc7c(c6)c6c(c(=O)n7C)OCC(F)(F)[C@H](C7CC7)N6)n5)CC4)CC3)cc21]

### Preparation of benzyl 4-(1-(tert-butoxycarbonyl)piperidine-4-carbonyl)piperazine-1-carboxylate

To a solution of 1-(tert-butoxycarbonyl)piperidine-4-carboxylic acid (5.0 g, 21.8 mmol) in DMF (25 mL) was added DIPEA (19.0 mL, 109.03 mmol), HOBt (6.67 g, 43.61 mmol), EDC-HCl (8.36 g, 43.61 mmol). The reaction was stirred for 15 minutes and cooled to 0° C., when benzyl piperazine-1-carboxylate (7.2 g, 32.71 mmol) was added. The resulting mixture was stirred at rt for 16 h. The reaction mixture was quenched with cold water (100 mL), and the solid precipitate thus formed was filtered and dried under reduced pressure to obtain benzyl 4-(1-(tert-butoxycarbonyl)piperidine-4-carbonyl)piperazine-1-carboxylate (3.0 g).

LC-MS (ESI): m/z=454.58 [M+H]⁺

### Preparation of tert-butyl 4-(2-(piperazin-1-yl)propan-2-yl)piperidine-1-carboxylate

To a stirred solution of zirconium (IV) tetrachloride (2.59 g, 11.12 mmol) in THF (15 mL) was added a solution of benzyl 4-(1-(tert-butoxycarbonyl)piperidine-4-carbonyl)piperazine-1-carboxylate (3.0 g, 6.95 mmol) in THF (16 mL) at −60° C. The reaction mixture stirred for 15 min.

Then, methyl magnesium chloride 3 M in THF (23 mL, 69.52 mmol) was added at −60° C., and the resulting mixture was stirred for 10 min. The resultant reaction mixture was stirred at −60° C. for 3 h. The reaction mixture was quenched with saturated solution of ammonium chloride (20 mL) and extracted with EtOAc (3×10 mL). The combined organic layer was dried with sodium sulfate, filtered, concentrated under vacuum, and purified by flash column chromatography (SiO₂, 100-200 mesh, 1-5% MeOH in DCM) to afford tert-butyl 4-(2-(piperazin-1-yl)propan-2-yl)piperidine-1-carboxylate brown semisolid (1.0 g).

LC-MS (ESI): m/z=312.41 [M+H]⁺.

### Preparation of tert-butyl 4-(2-(4-(3-(2,6-bis(benzyloxy)pyridin-3-yl)-1-methyl-1H-indazol-6-yl)piperazin-1-yl)propan-2-yl)piperidine-1-carboxylate

A solution of 3-(2,6-bis(benzyloxy)pyridin-3-yl)-6-bromo-1-methyl-1H-indazole (0.5 g, 0.99 mmol), tert-butyl 4-(2-(piperazin-1-yl)propan-2-yl)piperidine-1-carboxylate (1.24 g, 3.99 mmol), and potassium tert-butoxide (0.33 g, 2.99 mmol) in 1,4-dioxane (10 mL) was degassed with argon for 10 minutes. BrettPhos Pd-G3 (0.09 g, 0.01 mmol) was then added, and the mixture was stirred and heated at 100° C. for 16 h. The reaction mixture was diluted with ethyl acetate (200 mL) and filtered through a celite bed. The collected filtrate was concentrated under vacuum and purified by flash column chromatography (SiO₂, 230-400 mesh, 40% ethyl acetate in petroleum ether) to afford tert-butyl 4-(2-(4-(3-(2,6-bis(benzyloxy)pyridin-3-yl)-1-methyl-1H-indazol-6-yl)piperazin-1-yl)propan-2-yl)piperidine-1-carboxylate (0.38 g) as a pale yellow semi-solid.

LC-MS (ESI): m/z=731.82 [M+H]⁺.

### Preparation of tert-butyl 4-(2-(4-(3-(2,6-dioxopiperidin-3-yl)-1-methyl-1H-indazol-6-yl)piperazin-1-yl)propan-2-yl)piperidine-1-carboxylate

To a solution of tert-butyl 4-(2-(4-(3-(2,6-bis(benzyloxy)pyridin-3-yl)-1-methyl-1H-indazol-6-yl)piperazin-1-yl)propan-2-yl)piperidine-1-carboxylate (0.38 g, 0.52 mmol) in THF (7.6 mL) was added 20% Pd(OH)₂ on carbon (0.43 g, 100% w/w). The reaction mixture was stirred under hydrogen atmosphere (80 psi) for 16 h at room temperature. The reaction mixture was diluted with THF, filtered through a pad of celite, and washed with excess of 20% THF in DCM. The filtrate was collected and concentrated to afford tert-butyl 4-(2-(4-(3-(2,6-dioxopiperidin-3-yl)-1-methyl-1H-indazol-6-yl)piperazin-1-yl)propan-2-yl)piperidine-1-carboxylate as an off white solid (0.26 g) which was used in the next step without purification.

LC-MS (ESI): m/z=553.72 [M+H]⁺

### Preparation of 3-(1-methyl-6-(4-(2-(piperidin-4-yl)propan-2-yl)piperazin-1-yl)-1H-indazol-3-yl)piperidine-2,6-dione

To a solution of tert-butyl 4-(2-(4-(3-(2,6-dioxopiperidin-3-yl)-1-methyl-1H-indazol-6-yl)piperazin-1-yl)propan-2-yl)piperidine-1-carboxylate (0.26 g, 0.47 mmol) in DCM (3.0 mL) was added TFA (0.28 mL, 3.76 mmol). The reaction mixture was stirred at room temperature for 2 h.

The reaction mixture was concentrated under vacuum to afford crude 3-(1-methyl-6-(4-(2-(piperidin-4-yl)propan-2-yl)piperazin-1-yl)-1H-indazol-3-yl)piperidine-2,6-dione (0.2 g) as a brown semi-solid.

LC-MS (ESI): m/z=453.54 [M+H]⁺

### Preparation of 3-(6-(4-(2-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)propan-2-yl)piperazin-1-yl)-1-methyl-1H-indazol-3-yl)piperidine-2,6-dione

To a solution of 3-(1-methyl-6-(4-(2-(piperidin-4-yl)propan-2-yl)piperazin-1-yl)-1H-indazol-3-yl)piperidine-2,6-dione (0.1 g, 0.22 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.031 g, 0.11 mmol) in DMSO (2.0 mL) was added N,N-diisopropylethylamine (0.54 mL, 3.03 mmol). The reaction mixture was stirred at 100° C. for 6 h. The reaction mixture was quenched with ice-cold water (10 mL), and the solid precipitate was filtered and dried under vacuum to give a residue which was purified by prep-HPLC to afford 3-(6-(4-(2-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)propan-2-yl)piperazin-1-yl)-1-methyl-1H-indazol-3-yl)piperidine-2,6-dione (12 mg) as an off white solid.

LC-MS (ESI): m/z=884.45 [M+H]⁺

¹H NMR (400 MHz, DMSO-d₆): δ 10.85 (s, 1H), 8.76 (s, 1H), 8.21 (s, 1H), 8.02 (s, 1H), 7.73 (d, J=9.20 Hz, 1H), 7.47 (dd, J=9.20, 21.20 Hz, 2H), 6.91 (d, J=8.80 Hz, 1H), 6.81 (s, 1H), 6.19 (s, 1H), 4.59 (br, 2H), 4.42-4.39 (m, 2H), 4.25-4.24 (m, 1H), 3.88 (s, 3H), 3.55 (s, 3H), 3.23-3.17 (m, 4H), 2.71-2.59 (m, 8H), 2.44-2.42 (m, 1H), 2.18-2.12 (m, 2H), 1.81-1.73 (m, 6H), 1.32-1.24 (m, 2H), 1.24 (m, 2H), δ 1.12-1.10 (m, 1H), 0.71-0.70 (m, 1H), 0.53-0.50 (m, 2H), 0.37-0.36 (m, 1H).

### Example 116: 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 486d)

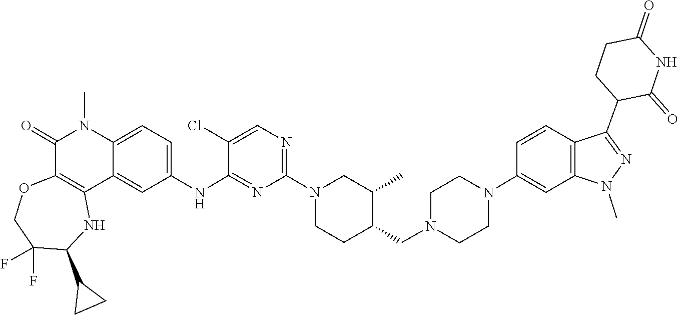 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCN(c2ccc3c(C4CCC(=O)NC4=O)nn(C)c3c2)CC1]

### Preparation of tert-butyl (3R, 4R)-4-(4-(3-(2,6-bis(benzyloxy)pyridin-3-yl)-1-methyl-1H-indazol-6-yl)piperazine-1-carbonyl)-3-methylpiperidine-1-carboxylate

To a solution of 3-(2,6-bis(benzyloxy)pyridin-3-yl)-1-methyl-6-(piperazin-1-yl)-1H-indazole (1.0 g, 1.97 mmol) and (3R, 4R)-1-(tert-butoxycarbonyl)-3-methylpiperidine-4-carboxylic acid (0.48 g, 1.97 mmol) in DMF (10 mL) was added DIPEA (1.79 g, 13.84 mmol) followed by HATU (1.12 g, 2.96 mmol) at 0° C. The resultant reaction mixture was stirred at room temperature for 1 h. The reaction mixture was quenched with cold water (20 mL), and the solid precipitate thus formed was filtered and dried under reduced pressure to obtain tert-butyl (3R, 4R)-4-(4-(3-(2,6-bis(benzyloxy)pyridin-3-yl)-1-methyl-1H-indazol-6-yl)piperazine-1-carbonyl)-3-methylpiperidine-1-carboxylate (1.3 g) as a white solid.

LC-MS (ESI): m/z=731.5 [M+H]⁺

### Preparation of tert-butyl (3R, 4R)-4-((4-(3-(2,6-bis(benzyloxy)pyridin-3-yl)-1-methyl-1H-indazol-6-yl)piperazin-1-yl)methyl)-3-methylpiperidine-1-carboxylate

To a solution of tert-butyl (3R, 4R)-4-(4-(3-(2,6-bis(benzyloxy)pyridin-3-yl)-1-methyl-1H-indazol-6-yl)piperazine-1-carbonyl)-3-methylpiperidine-1-carboxylate (1.3 g, 1.77 mmol) in THF (39 mL) was added BH₃ in DMS (104 mL) at 0° C. The resultant reaction mixture was stirred at room temperature for 16 h. The reaction mass was quenched with methanol (100 mL) slowly and concentrated under reduced pressure to get a residue which was purified by flash column chromatography (20% EtOAc in petroleum ether) to provide t tert-butyl (3R, 4R)-4-((4-(3-(2,6-bis(benzyloxy)pyridin-3-yl)-1-methyl-1H-indazol-6-yl)piperazin-1-yl)methyl)-3-methylpiperidine-1-carboxylate (1.2 g) as a white semi-solid.

LC-MS (ESI): m/z=717.53 [M+H⁺]

### Preparation of tert-butyl (3R, 4R)-4-((4-(3-(2,6-dioxopiperidin-3-yl)-1-methyl-1H-indazol-6-yl)piperazin-1-yl)methyl)-3-methylpiperidine-1-carboxylate

To a solution of tert-butyl (3R, 4R)-4-((4-(3-(2,6-bis(benzyloxy)pyridin-3-yl)-1-methyl-1H-indazol-6-yl)piperazin-1-yl)methyl)-3-methylpiperidine-1-carboxylate (1.2 g, 1.67 mmol) in THF (48 mL) was added 20% Pd(OH)₂ on carbon (4.69 g). The reaction mixture was stirred under hydrogen atmosphere (80 psi) at room temperature for 16 h. The reaction mixture was filtered through a celite bed and washed with 20% THF in DCM (3×100 mL). The combined organic layer was concentrated to provide a crude product which was purified by triturating with diethyl ether followed by n-pentane to afford tert-butyl (3R, 4R)-4-((4-(3-(2,6-dioxopiperidin-3-yl)-1-methyl-1H-indazol-6-yl)piperazin-1-yl)methyl)-3-methylpiperidine-1-carboxylate as a brown semi-solid (0.62 g)

LC-MS (ESI): m/z=539.65 [M+H]⁺

### Preparation of 3-(1-methyl-6-(4-(((3R, 4R)-3-methylpiperidin-4-yl)methyl)piperazin-1-yl)-1H-indazol-3-yl)piperidine-2,6-dione

To a solution of tert-butyl (3R, 4R)-4-((4-(3-(2,6-dioxopiperidin-3-yl)-1-methyl-1H-indazol-6-yl)piperazin-1-yl)methyl)-3-methylpiperidine-1-carboxylate (0.68 g, 1.26 mmol) in DCM (6.8 mL) was added TFA (0.77 mL, 10.09 mmol). The reaction mixture was stirred at room temperature for 2 h. The reaction mixture was concentrated under vacuum to provide a crude residue which was triturated by diethyl ether (5 mL) to afford 3-(1-methyl-6-(4-(((3R, 4R)-3-methylpiperidin-4-yl)methyl)piperazin-1-yl)-1H-indazol-3-yl)piperidine-2,6-dione (0.5 g) as a brown semi-solid.

LC-MS (ESI): m/z=439.28 [M+H]⁺.

### Preparation of 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-6-(4-(((3R, 4R)-3-methylpiperidin-4-yl)methyl)piperazin-1-yl)-1H-indazol-3-yl)piperidine-2,6-dione (0.15 g, 0.34 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.04 g, 0.1 mmol) in DMSO (1.5 mL) was added N,N-diisopropylethylamine (0.49 mL, 2.73 mmol). The reaction mixture was stirred at 100° C. for 5 h.

The reaction mixture was poured into ice cold water and stirred for 15 minutes. The resulting precipitate was filtered and dried to provide a crude product, which was purified by prep-HPLC to afford 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (0.013 g) as a pale brown solid.

¹H NMR (400 MHz, DMSO-d6): δ 10.85 (s, 1H), 8.74 (s, 1H), 8.26 (s, 1H), 8.00 (s, 1H), 7.76 (d, J=8.80 Hz, 1H), 7.50-7.44 (m, 2H), 6.92-6.89 (m, 1H), 6.83 (s, 1H), 6.18 (s, 1H), 4.41-4.24 (m, 5H), 3.89 (s, 3H), 3.56 (s, 3H), 3.34-3.02 (m, 5H), 2.93 (m, 1H), 2.67 (m, 1H), 2.64-2.50 (m, 6H), 2.29-2.13 (m, 4H), 1.93-1.90 (m, 2H), 1.41-1.33 (m, 3H), 0.77-0.71 (m, 4H), 0.53-051 (m, 2H), 0.35-034 (m, 1H).

LC-MS (ESI): m/z=870.46 [M+H]⁺.

### Example 117: 3-(6-(4-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 486c)

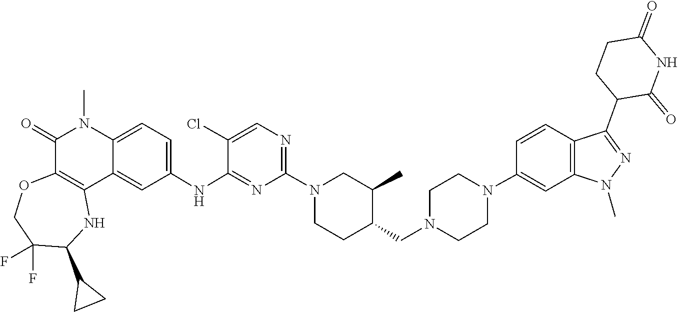 [SMILES: C[C@@H]1CN(c2ncc(Cl)c(Nc3ccc4c(c3)c3c(c(=O)n4C)OCC(F)(F)[C@H](C4CC4)N3)n2)CC[C@H]1CN1CCN(c2ccc3c(C4CCC(=O)NC4=O)nn(C)c3c2)CC1]

### Preparation of 3-(2,6-bis(benzyloxy)pyridin-3-yl)-1-methyl-6-(piperazin-1-yl)-1H-indazole

To a solution of 3-(2,6-bis(benzyloxy)pyridin-3-yl)-6-bromo-1-methyl-1H-indazole (5.0 g, 9.99 mmol) and piperazine (1.80 g, 20.98 mmol) in 1,4-dioxane (100 mL) was added sodium tert-butoxide (2.88 g, 29.97 mmol). The reaction mixture was degassed with argon for 15 minutes.

Then Ruphos (0.46 g, 0.99 mmol) and Ruphos Pd G3 (0.41 g, 0.49 mmol) were added, and the reaction was stirred at 100° C. for 16 h. The reaction mixture was diluted with EtOAc (50 mL), filtered through a celite bed, and washed with ethyl acetate (100 mL). The collected filtrate was concentrated under reduced pressure to provide a crude product that was purified by flash column chromatography (SiO₂, 100-200 mesh, 50% ethyl acetate in petroleum ether) to afford 3-(2,6-bis(benzyloxy)pyridin-3-yl)-1-methyl-6-(piperazin-1-yl)-1H-indazole (2.5 g) as a brown semi-solid.

LC-MS (ESI): m/z=506.57 [M+H]⁺.

### Preparation of tert-butyl (3S, 4R)-4-(4-(3-(2,6-bis(benzyloxy)pyridin-3-yl)-1-methyl-1H-indazol-6-yl)piperazine-1-carbonyl)-3-methylpiperidine-1-carboxylate

To a solution of 3-(2,6-bis(benzyloxy)pyridin-3-yl)-1-methyl-6-(piperazin-1-yl)-1H-indazole (1.0 g, 2.05 mmol) and (3S, 4R)-1-(tert-butoxycarbonyl)-3-methylpiperidine-4-carboxylic acid (0.5 g, 2.05 mmol) in THF (20 mL) was added DIPEA (1.4 mL, 8.22 mmol). The reaction mixture was stirred for 10 min. HATU (1.2 g, 3.08 mmol) was then added at 0° C., and the reaction mixture was stirred at rt for 2 h. After completion, the reaction was diluted with ethyl acetate (30 mL) and washed with cold water (3×20 mL). The organic layer was dried over anhydrous sodium sulfate and concentrated under reduced pressure to provide a residue which was triturated using n-pentane to afford tert-butyl (3S, 4R)-4-(4-(3-(2,6-bis(benzyloxy)pyridin-3-yl)-1-methyl-1H-indazol-6-yl)piperazine-1-carbonyl)-3-methylpiperidine-1-carboxylate (1.1 g) as an off white solid.

LC-MS (ESI): m/z=731.50 [M+H]⁺.

### Preparation of tert-butyl (3S, 4R)-4-((4-(3-(2,6-bis(benzyloxy)pyridin-3-yl)-1-methyl-1H-indazol-6-yl)piperazin-1-yl)methyl)-3-methylpiperidine-1-carboxylate

To a solution of tert-butyl (3S, 4R)-4-(4-(3-(2,6-bis(benzyloxy)pyridin-3-yl)-1-methyl-1H-indazol-6-yl)piperazine-1-carbonyl)-3-methylpiperidine-1-carboxylate (1.3 g, 1.77 mmol) in THF (26 mL) was added 2.0 M borane dimethylsulfide solution in THF (26 mL). The reaction mixture was stirred at rt for 8 h. After completion, the reaction mixture was quenched with methanol and evaporated under reduced pressure. The residue was dissolved in methanol and refluxed at 80° C. for 2 h. The reaction mixture was concentrated under reduced pressure to provide a crude product that was purified by flash column chromatography (SiO₂, 100-200 mesh, 50% ethyl acetate in petroleum ether) to afford tert-butyl (3S, 4R)-4-((4-(3-(2,6-bis(benzyloxy)pyridin-3-yl)-1-methyl-1H-indazol-6-yl)piperazin-1-yl)methyl)-3-methylpiperidine-1-carboxylate (0.8 g) as a brown semi-solid.

LC-MS (ESI): m/z=717.83 [M+H]⁺

### Preparation of tert-butyl (3S, 4R)-4-((4-(3-(2,6-dioxopiperidin-3-yl)-1-methyl-1H-indazol-6-yl)piperazin-1-yl)methyl)-3-methylpiperidine-1-carboxylate

To a solution of tert-butyl (3S, 4R)-4-((4-(3-(2,6-bis(benzyloxy)pyridin-3-yl)-1-methyl-1H-indazol-6-yl)piperazin-1-yl)methyl)-3-methylpiperidine-1-carboxylate (0.80 g, 1.12 mmol) in THF (24.0 mL) was added 20% Pd(OH)₂ on carbon (0.80 g, 100% w/w). The reaction mixture was stirred under hydrogen atmosphere (80 psi) at room temperature for 16 h. The reaction mixture diluted with ethyl acetate (50 mL), filtered through celite bed, and washed with 50% THF in ethyl acetate (200 mL). The collected filtrate was concentrated under reduced pressure to get a crude product that was triturated by using diethyl ether to afford tert-butyl (3S, 4R)-4-((4-(3-(2,6-dioxopiperidin-3-yl)-1-methyl-1H-indazol-6-yl)piperazin-1-yl)methyl)-3-methylpiperidine-1-carboxylate (0.60 g) as a brown semi-solid.

LC-MS (ESI): m/z=539.41 [M+H]⁺.

### Preparation of 3-(1-methyl-6-(4-(((3S, 4R)-3-methylpiperidin-4-yl)methyl)piperazin-1-yl)-1H-indazol-3-yl)piperidine-2,6-dione

To a solution of tert-butyl (3S, 4R)-4-((4-(3-(2,6-dioxopiperidin-3-yl)-1-methyl-1H-indazol-6-yl)piperazin-1-yl)methyl)-3-methylpiperidine-1-carboxylate (0.60 g, 1.11 mmol) in DCM (6.0 mL) was added TFA (3.0 mL). The reaction mixture was stirred at room temperature for 1 h. The reaction mixture was concentrated and co-distilled with petroleum ether under reduced pressure to get a crude product that was triturated by using diethyl ether to afford 3-(1-methyl-6-(4-(((3S, 4R)-3-methylpiperidin-4-yl)methyl)piperazin-1-yl)-1H-indazol-3-yl)piperidine-2,6-dione (0.5 g) as an off white solid.

LC-MS (ESI): m/z=439.32 [M+H]⁺.

### Preparation of 3-(6-(4-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-6-(4-(((3S, 4R)-3-methylpiperidin-4-yl)methyl)piperazin-1-yl)-1H-indazol-3-yl)piperidine-2,6-dione (0.25 g, 0.57 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.17 mmol) in DMSO (5 mL) was added N,N-diisopropylethylamine (0.5 mL, 2.85 mmol). The resulting reaction mixture was stirred at 100° C. for 6 h. The reaction mixture was poured into ice-cold water and stirred for 15 minutes, and the resulting precipitate was filtered and dried to provide a crude product that was purified by prep-HPLC to afford 3-(6-(4-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (45 mg) as a white solid.

LC-MS (ESI): m/z=870.42 [M+H]⁺

¹H NMR (400 MHz, DMSO-d₆): δ 10.84 (s, 1H), 8.78 (s, 1H), 8.31 (s, 1H), 8.17 (s, 1H), 8.02 (s, 1H), 7.74 (dd, J=2.00, 9.00 Hz, 1H), 7.46 (dd, J=9.20, 22.20 Hz, 2H), 6.90 (d, J=9.20 Hz, 1H), 6.82 (s, 1H), 6.16 (s, 1H), 4.27-4.26 (m, 5H), 3.88 (s, 3H), 3.56 (s, 3H), 3.23-3.22 (br, 4H), 2.82-2.80 (m, 1H), 2.62-2.60 (m, 4H), 2.50-2.49 (m, 4H), 2.29-2.18 (m, 1H), 2.11-1.90 (m, 2H), 1.99-1.81 (m, 1H), 1.50-1.24 (m, 3H), 1.05-0.99 (m, 1H), 0.89 (d, J=6 Hz, 3H), 0.75-0.65 (m, 1H), 0.60-0.40 (m, 2H), 0.45-0.25 (m, 1H).

### Example 118: 3-(7-(4-(((2R, 5S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazin-1-yl)methyl)piperidin-1-yl)-1-methyl-1H-indazol-3-yl)piperidine-2,6-dione (Compound 491b)

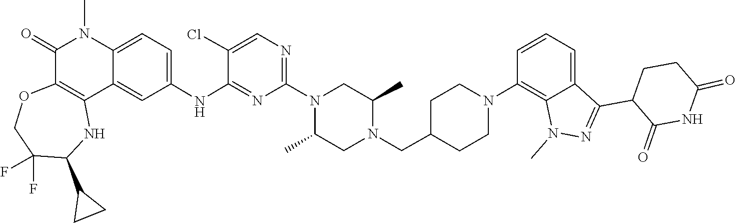 [SMILES: C[C@@H]1CN(c2ncc(Cl)c(Nc3ccc4c(c3)c3c(c(=O)n4C)OCC(F)(F)[C@H](C4CC4)N3)n2)[C@@H](C)CN1CC1CCN(c2cccc3c(C4CCC(=O)NC4=O)nn(C)c23)CC1]

### Preparation of tert-butyl (2S, 5R)-4-((1-((benzyloxy)carbonyl)piperidin-4-yl)methyl)-2,5-dimethylpiperazine-1-carboxylate

To a solution tert-butyl (2S, 5R)-2,5-dimethylpiperazine-1-carboxylate (3.0 g, 13.99 mmol) and benzyl 4-formylpiperidine-1-carboxylate (3.46 g, 13.99 mmol) in DCM (60 mL) was added acetic acid (0.8 mL, 13.99 mmol) at 0° C. The reaction mixture was stirred for 2 h. NaBH(OAc)₃ (3.56 g, 16.79 mmol) was then added at 0° C., and the reaction mixture was stirred at room temperature for 16 h. The reaction mixture was quenched with water (100 ml) and extracted with DCM (150 mL×3). The separated organic layer was dried over anhydrous sodium sulfate, filtered, and concentrated to provide a crude product which was triturated with n-pentane to afford tert-butyl (2S, 5R)-4-((1-((benzyloxy)carbonyl)piperidin-4-yl)methyl)-2,5-dimethylpiperazine-1-carboxylate (5.0 g).

LC-MS (ESI): m/z=446.99 [M+H]⁺

### Preparation of tert-butyl (2S, 5R)-2,5-dimethyl-4-(piperidin-4-ylmethyl)piperazine-1-carboxylate

To a solution of tert-butyl (2S, 5R)-4-((1-((benzyloxy)carbonyl)piperidin-4-yl)methyl)-2,5-dimethylpiperazine-1-carboxylate (3.0 g, 6.73 mmol) in THF (90.0 mL) was added 10% palladium on carbon (3.0 g, 100% w/w). The reaction mixture was stirred under hydrogen atmosphere (70 psi) at room temperature for 16 h. The reaction mixture was filtered through a celite bed and concentrated to provide a crude product which was washed with n-pentane and dried under vacuum to afford tert-butyl (2S, 5R)-2,5-dimethyl-4-(piperidin-4-ylmethyl)piperazine-1-carboxylate (2.0 g) as a brown solid which was used in the next step without further purification.

LC-MS (ESI): m/z=312.61 [M+H]⁺.

### Preparation of tert-butyl (2S, 5R)-4-((1-(3-(2,6-bis(benzyloxy)pyridin-3-yl)-1-methyl-1H-indazol-7-yl)piperidin-4-yl)methyl)-2,5-dimethylpiperazine-1-carboxylate

To a stirred solution of 3-(2,6-bis(benzyloxy)pyridin-3-yl)-7-bromo-1-methyl-1H-indazole (1.5 g, 2.99 mmol), tert-butyl (2S, 5R)-2,5-dimethyl-4-(piperidin-4-ylmethyl)piperazine-1-carboxylate (1.86 g, 5.98 mmol), and cesium carbonate (2.92 g, 8.97 mmol) in 1,4-dioxane (15.0 mL) and DMF (15.0 mL) was added Pd-PEPPSI-iHeptCl (0.14 g, 0.15 mmol) under argon. The reaction mixture was heated to 100° C. for 18 h. The reaction mixture was filtered through a pad of celite bed and washed with EtOAc (30 mL) and water (20 mL). The organic layers were dried over sodium sulfate and concentrated under vacuum to obtain crude product, which was purified by flash chromatography (15% of ethyl acetate in petroleum ether) to afford tert-butyl (2S, 5R)-4-((1-(3-(2,6-bis(benzyloxy)pyridin-3-yl)-1-methyl-1H-indazol-7-yl)piperidin-4-yl)methyl)-2,5-dimethylpiperazine-1-carboxylate (1.0 g) as a pale yellow solid.

LC-MS (ESI): m/z=731.81 [M+H]⁺

### Preparation of tert-butyl (2S, 5R)-4-((1-(3-(2,6-dioxopiperidin-3-yl)-1-methyl-1H-indazol-7-yl)piperidin-4-yl)methyl)-2,5-dimethylpiperazine-1-carboxylate

To a solution tert-butyl (2S, 5R)-4-((1-(3-(2,6-bis(benzyloxy)pyridin-3-yl)-1-methyl-1H-indazol-7-yl)piperidin-4-yl)methyl)-2,5-dimethylpiperazine-1-carboxylate (1.0 g, 1.36 mmol) in THF (40 mL) was added 20% Pd(OH)₂ on carbon (1.0 g). The reaction mixture was stirred under H₂ atmosphere (80 psi) with for 16 h at room temperature. The reaction mixture was diluted with DCM (100 mL) and washed with 50% THF:DCM (300 mL) and filtered through a celite bed. The collected filtrate was concentrated under vacuum to afford tert-butyl (2S, 5R)-4-((1-(3-(2,6-dioxopiperidin-3-yl)-1-methyl-1H-indazol-7-yl)piperidin-4-yl)methyl)-2,5-dimethylpiperazine-1-carboxylate (0.75 g) as a pale brown semi-solid which was used in the next step without purification.

LC-MS (ESI): m/z=553.72 [M+H]⁺

### Preparation of 3-(7-(4-(((2R, 5S)-2,5-dimethylpiperazin-1-yl)methyl)piperidin-1-yl)-1-methyl-1H-indazol-3-yl)piperidine-2,6-dione

To a solution of tert-butyl (2S, 5R)-4-((1-(3-(2,6-dioxopiperidin-3-yl)-1-methyl-1H-indazol-7-yl)piperidin-4-yl)methyl)-2,5-dimethylpiperazine-1-carboxylate (0.75 g, 1.35 mmol) in DCM (8.0 mL) was added trifluoroacetic acid (7.5 mL) at 0° C. The resulting mixture was stirred at room temperature for 2 h. The reaction mixture was concentrated under reduced pressure to afford 3-(7-(4-(((2R, 5S)-2,5-dimethylpiperazin-1-yl)methyl)piperidin-1-yl)-1-methyl-1H-indazol-3-yl)piperidine-2,6-dione (0.75 g) as a white solid which was used in the next step without further purification.

LC-MS (ESI): m/z=453.59 [M+H]⁺

### Preparation of 3-(7-(4-(((2R, 5S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazin-1-yl)methyl)piperidin-1-yl)-1-methyl-1H-indazol-3-yl)piperidine-2,6-dione

To a stirred solution of 3-(7-(4-(((2R, 5S)-2,5-dimethylpiperazin-1-yl)methyl)piperidin-1-yl)-1-methyl-1H-indazol-3-yl)piperidine-2,6-dione (0.20 g, 0.44 mmol), and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.17 mmol) in DMSO (4.0 mL) was added DIPEA (0.6 mL, 3.52 mmol), and the resulting reaction mixture was heated to 100° C. for 24 h.

The reaction mixture was to added ice-cold water, filtered, and dried to obtain crude product which was purified by prep-SFC to afford 3-(7-(4-(((2R, 5S)-4-(5-chloro-4-(((S)-2-cyclopropyl-3,3-difluoro-7-methyl-6-oxo-1,2,3,4,6,7-hexahydro-[1,4]oxazepino [2,3-c]quinolin-10-yl)amino)pyrimidin-2-yl)-2,5-dimethylpiperazin-1-yl)methyl)piperidin-1-yl)-1-methyl-1H-indazol-3-yl)piperidine-2,6-dione (8 mg) as an off white solid. ¹H NMR: (400 MHz, DMSO-d6): δ 10.87 (s, 1H), 8.75 (s, 1H), 8.17 (s, 1H), 8.03 (s, 1H), 7.73 (dd, J=1.60, 9.00 Hz, 1H), 7.44 (d, J=9.20 Hz, 1H), 7.36-7.35 (m, 1H), 7.01-7.00 (m, 2H), 6.19 (s, 1H), 4.56 (brs, 1H), 4.32-4.31 (m, 3H), 4.24 (s, 3H), 4.16 (d, J=17.0 Hz, 1H), 3.57 (s, 3H), 3.23-3.20 (m, 4H), 2.94 (brs, 1H), 2.62-2.61 (m, 4H), 2.19-2.17 (m, 5H), 1.91 (s, 2H), 1.62 (brs, 1H), 134-1.32 (m, 4H), 1.16 (d, J=6.40 Hz, 3H), 0.83 (d, J=6.40 Hz, 3H), 0.70 (s, 1H), 0.52-0.50 (m, 2H), 0.36-0.35 (m, 1H).

LC-MS (ESI): m/z=884.52 [M+H]⁺.

### Example 119: 3-(7-(4-(((2S, 5R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazin-1-yl)methyl)piperidin-1-yl)-1-methyl-1H-indazol-3-yl)piperidine-2,6-dione (Compound 491a)

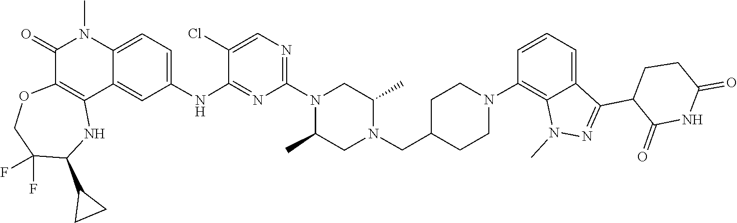 [SMILES: C[C@@H]1CN(CC2CCN(c3cccc4c(C5CCC(=O)NC5=O)nn(C)c34)CC2)[C@@H](C)CN1c1ncc(Cl)c(Nc2ccc3c(c2)c2c(c(=O)n3C)OCC(F)(F)[C@H](C3CC3)N2)n1]

### Preparation of tert-butyl (2S, 5R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazine-1-carboxylate

To a stirred solution of tert-butyl (2S, 5R)-2,5-dimethylpiperazine-1-carboxylate (0.45 g, 2.10 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.29 g, 0.63 mmol) in DMSO (4.5 mL) was added N,N-Diisopropylethylamine (1.79 mL, 10.50 mmol). The reaction mixture was heated to 100° C. for 6 h. The reaction mixture was quenched with ice-cold water (200 mL), and the precipitated solid was filtered and dried under reduced pressure to afford tert-butyl (2S, 5R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazine-1-carboxylate (0.60 g) as a brown solid which was used in the next step without further purification.

LC-MS (ESI): m/z=646.39 [M+H]⁺.

### Preparation of (S)-10-((5-chloro-2-((2R, 5S)-2,5-dimethylpiperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one

To a solution of tert-butyl (2S, 5R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazine-1-carboxylate (0.55 g, 0.85 mmol) in DCM (11.0 mL) was added TFA (2.75 mL) at 0° C. The reaction mixture was stirred at room temperature for 2 h. The reaction mixture was concentrated and co-distilled with DCM (3×40 mL) and dried to afford (S)-10-((5-chloro-2-((2R, 5S)-2,5-dimethylpiperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one (0.55 g) as a brown solid which was used in the next step without further purification.

LC-MS (ESI): m/z=546.27 [M+H]⁺.

### Preparation of (1-(3-(2,6-bis(benzyloxy)pyridin-3-yl)-1-methyl-1H-indazol-7-yl)piperidin-4-yl)methanol

To a solution of 3-(2,6-bis(benzyloxy)pyridin-3-yl)-7-bromo-1-methyl-1H-indazole (2.0 g, 4.01 mmol), piperidin-4-ylmethanol (1.84 g, 16.03 mmol) and cesium carbonate (3.9 g, 12.02 mmol) in 1,4-dioxane (20 mL) was added Pd-PEPPSI-IHeptCl (0.40 g, 0.40 mmol) under argon. The resulting mixture was stirred and heated to 90° C. for 6 h. The reaction mixture was cooled to room temperature and filtered through a pad of celite bed. The collected filtrate was concentrated to obtain crude product that was purified by flash column chromatography (SiO₂, 230-400 mesh, 80% ethyl acetate in petroleum ether) to afford (1-(3-(2,6-bis(benzyloxy)pyridin-3-yl)-1-methyl-1H-indazol-7-yl)piperidin-4-yl)methanol (1.50 g) as a yellow solid.

LCMS (ESI): m/z=535.64 [M+H]⁺.

### Preparation of 3-(7-(4-(hydroxymethyl)piperidin-1-yl)-1-methyl-1H-indazol-3-yl)piperidine-2,6-dione

To a solution of (1-(3-(2,6-bis(benzyloxy)pyridin-3-yl)-1-methyl-1H-indazol-7-yl)piperidin-4-yl)methanol (1.50 g, 0.003 mmol) in THF (75 mL) was added 20% Pd(OH)₂ on carbon (3.0 g).

The reaction mixture was stirred under H₂ (70 psi) atmosphere for 16 h. The reaction mixture was filtered through a celite bed and washed with THF:DCM (1:1) (1000 mL). The collected filtrate was concentrated under reduced pressure to afford 3-(7-(4-(hydroxymethyl)piperidin-1-yl)-1-methyl-1H-indazol-3-yl)piperidine-2,6-dione (0.90 g) as an off-white solid which was used in the next step without purification.

LC-MS (ESI): m/z=357.19 [M+H]⁺.

### Preparation of 1-(3-(2,6-dioxopiperidin-3-yl)-1-methyl-1H-indazol-7-yl)piperidine-4-carbaldehyde

To a solution of 3-(7-(4-(hydroxymethyl)piperidin-1-yl)-1-methyl-1H-indazol-3-yl)piperidine-2,6-dione (0.70 g, 1.97 mmol) in DCM (21 mL) was added Dess-Martin periodinane (3.93 g, 1.67 mmol) at 0° C. The reaction mixture was stirred at room temperature for 4 h. The reaction mixture was diluted with DCM (50 mL), filtered under vacuum, and washed with excess of DCM (50 mL).

The collected filtrate was washed with saturated solution of NaHCO₃ in water (100 mL×2), and the separated organic layer was dried over anhydrous Na₂SO₄, filtered, and dried under vacuum to afford 1-(3-(2,6-dioxopiperidin-3-yl)-1-methyl-1H-indazol-7-yl)piperidine-4-carbaldehyde (0.60 g) as a brown solid which was used in the next step without purification.

LC-MS (ESI): m/z=355.41 [M+H]⁺

### Preparation of 3-(7-(4-(((2S, 5R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazin-1-yl)methyl)piperidin-1-yl)-1-methyl-1H-indazol-3-yl)piperidine-2,6-dione

A solution of 1-(3-(2,6-dioxopiperidin-3-yl)-1-methyl-1H-indazol-7-yl)piperidine-4-carbaldehyde (0.30 g, 0.85 mmol), (S)-10-((5-chloro-2-((2R, 5S)-2,5-dimethylpiperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one (0.46 g, 0.85 mmol), CH₃COONa (0.21 g, 2.54 mmol) and AcOH (0.3 mL) in THF (6 mL) was stirred at room temperature for 2 h. To the resulting solution, sodium triacetoxyborohydride (0.54 g, 2.54 mmol) was added at 0° C. The reaction mixture was stirred at room temperature for 4 h. The reaction mixture was quenched with water (50 mL) and extracted with ethyl acetate (2×50 mL). The combined organic layers were washed with brine solution (30 mL), dried over anhydrous Na₂SO₄, filtered, and concentrated under reduced pressure to provide a crude product that was purified by prep-HPLC to afford 3-(7-(4-(((2S, 5R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-dimethylpiperazin-1-yl)methyl)piperidin-1-yl)-1-methyl-1H-indazol-3-yl)piperidine-2,6-dione (0.06 g) as an off white solid.

LC-MS (ESI): m/z=882.39 [M−H]⁻.

¹H NMR (400 MHz, DMSO-d₆): δ 10.88 (s, 1H), 9.06-9.01 (m, 2H), 8.10-8.07 (m, 2H), 7.67 (d, J=9.20 Hz, 1H), 7.45 (d, J=9.20 Hz, 1H), 7.39-7.38 (m, 1H), 7.02-7.00 (m, 2H), 6.31 (br s, 1H), 4.46 (br s, 1H), 4.41-4.39 (m, 4H), 4.24 (d, J=8.40 Hz, 3H), 3.67 (s, 3H), 3.57-3.56 (m, 2H), 3.43-3.40 (m, 7H), 2.63-2.61 (m, 2H), 2.19-2.18 (m, 1H), 1.94-1.84 (m, 4H), 1.56 (br, 3H), 1.32-1.14 (m, 7H), 0.72-0.71 (m, 1H), 0.52-0.50 (m, 2H), 0.34-0.32 (m, 1H).

### Example 120: 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-3,9-diazaspiro[5.5]undecan-3-yl)methyl)piperidin-1-yl)-1-methyl-1H-indazol-3-yl)piperidine-2,6-dione (Compound 494a)

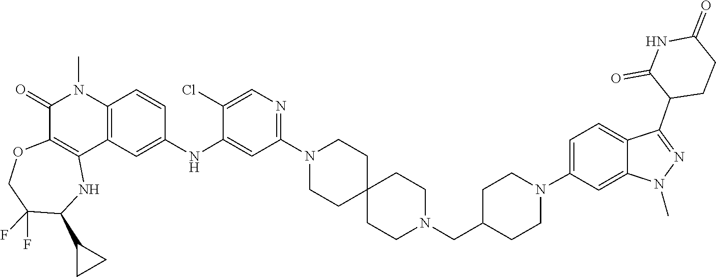 [SMILES: Cn1nc(C2CCC(=O)NC2=O)c2ccc(N3CCC(CN4CCC5(CC4)CCN(c4cc(Nc6ccc7c(c6)c6c(c(=O)n7C)OCC(F)(F)[C@H](C7CC7)N6)c(Cl)cn4)CC5)CC3)cc21]

### Preparation of tert-butyl 9-(5-chloro-4-iodopyridin-2-yl)-3,9-diazaspiro[5.5]undecane-3-carboxylate

To a stirred solution of tert-butyl 3,9-diazaspiro[5.5]undecane-3-carboxylate (1.0 g, 0.004 mmol) and 5-chloro-2-fluoro-4-iodopyridine (1.00 g, 0.004 mmol) in DMSO (20 mL) was added N,N-diisopropylethylamine (3.4 mL, 0.019 mmol). The reaction mixture was stirred at room temperature for 3 h. The reaction mixture was quenched with ice-cold water (400 mL), and the precipitated solid was filtered and dried under reduced pressure to afford tert-butyl 9-(5-chloro-4-iodopyridin-2-yl)-3,9-diazaspiro[5.5]undecane-3-carboxylate (1.20 g) as an off white solid which was used in the next step without purification.

LC-MS (ESI): m/z=492.43 [M+H]⁺.

### Preparation of tert-butyl (S)-9-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-3,9-diazaspiro[5.5]undecane-3-carboxylate

To a solution of tert-butyl 9-(5-chloro-4-iodopyridin-2-yl)-3,9-diazaspiro[5.5]undecane-3-carboxylate (0.40 g, 0.81 mmol) and (S)-10-amino-2-cyclopropyl-3,3-difluoro-7-methyl-1,2,3,4-tetrahydro-[1,4]oxazepino[2,3-c]quinolin-6(7H)-one (0.21 g, 0.65 mmol) in 1,4-dioxane (8 mL) was added cesium carbonate (0.79 g, 2.44 mmol) under argon. RuPhos (0.04 g, 0.08 mmol) and RuPhos-PdG3 (0.03 g, 0.04 mmol) was then added and the reaction mixture was stirred at 90° C. for 6 h. The reaction mixture was cooled to room temperature and filtered through a celite bed, and the filtrate was concentrated to get the crude product that was purified by flash column chromatography (SiO₂, mesh 230-400, 85% ethyl acetate in petroleum ether) to afford tert-butyl (S)-9-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-3,9-diazaspiro[5.5]undecane-3-carboxylate (0.55 g) as a brown solid.

LC-MS (ESI): m/z=685.6 [M+H]⁺

### Preparation of (S)-10-((5-chloro-2-(3,9-diazaspiro[5.5]undecan-3-yl)pyridin-4-yl)amino)-2-cyclopropyl-3,3-difluoro-7-methyl-1,2,3,4-tetrahydro-[1,4]oxazepino[2,3-c]quinolin-6(7H)-one

To a solution of tert-butyl (S)-9-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-3,9-diazaspiro[5.5]undecane-3-carboxylate (0.55 g, 0.80 mmol) in DCM (11 mL) was added TFA (2.75 mL) at 0° C. The reaction mixture was stirred at room temperature for 3 h. The reaction mixture was concentrated and co-distilled with DCM (3×40 mL) and dried to afford (S)-10-((5-chloro-2-(3,9-diazaspiro[5.5]undecan-3-yl)pyridin-4-yl)amino)-2-cyclopropyl-3,3-difluoro-7-methyl-1,2,3,4-tetrahydro-[1,4]oxazepino[2,3-c]quinolin-6(7H)-one (0.05 g) as a brown solid which was used in the next step without purification.

LC-MS (ESI): m/z=585.65 [M+H]⁺

### Preparation of benzyl 4-(1,3-dioxolan-2-yl)piperidine-1-carboxylate

To a solution of benzyl 4-formylpiperidine-1-carboxylate (5.0 g, 0.020 mmol) in toluene (100 mL) was added PTSA (0.38 g, 0.002 mmol). The reaction mixture was stirred for 10 min. Ethylene glycol (1.3 mL g, 0.024 mmol) was then added slowly at room temperature, and the resulting mixture was stirred for 12 h. After completion, the reaction mixture was concentrated under reduced pressure and triturated with n-pentane to afford benzyl 4-(1,3-dioxolan-2-yl)piperidine-1-carboxylate (3.20 g) which was used in the next step without further purification.

LCMS (ESI): m/z=292.41 [M+H]⁺.

### Preparation of 4-(1,3-dioxolan-2-yl)piperidine

To a solution of benzyl 4-(1,3-dioxolan-2-yl)piperidine-1-carboxylate (3.20 g, 0.010 mmol) in THF (160 mL) was added 20% Pd(OH)₂ on carbon (1.60 g). The reaction mixture was stirred under H₂ (80 psi) atmosphere at room temperature for 12 h. The reaction mixture was filtered through a celite bed and washed with THF:DCM (1:1) (1000 mL). The collected filtrate was concentrated under reduced pressure to afford 4-(1,3-dioxolan-2-yl)piperidine (1.40 g) as a pale yellow solid.

¹H NMR (400 MHz, DMSO-d₆): δ 4.51 (d, J=4.80 Hz, 1H), 3.84-3.83 (m, 2H), 3.80-3.79 (m, 2H), 2.91 (d, J=11.60 Hz, 2H), 2.38-2.37 (m, 2H), 1.51-1.50 (m, 3H), 1.36 (s, 1H), 1.13-1.12 (m, 2H).

### Preparation of 6-(4-(1,3-dioxolan-2-yl)piperidin-1-yl)-3-(2,6-bis(benzyloxy)pyridin-3-yl)-1-methyl-1H-indazole

To a stirred solution of 3-(2,6-bis(benzyloxy)pyridin-3-yl)-6-bromo-1-methyl-1H-indazole (2.30 g, 4.60 mmol) and 4-(1,3-dioxolan-2-yl)piperidine (1.45 g, 9.21 mmol) in 1,4-dioxane (46 mL) was added NaOtBu (1.33 g, 13.80 mmol). The reaction mixture was degassed with argon for 10 minutes. Then Brettphos-pd-G3 (0.42 g, 0.46 mmol) was added, and the reaction mixture was stirred at 90° C. for 4 h. The reaction mixture was cooled to room temperature and filtered through a celite bed. The filtrate was concentrated to provide a crude product that was purified by flash column chromatography (SiO₂, mesh 230-400 mesh, 50% ethyl acetate in petroleum ether) to afford 6-(4-(1,3-dioxolan-2-yl)piperidin-1-yl)-3-(2,6-bis(benzyloxy)pyridin-3-yl)-1-methyl-1H-indazole (1.80 g) as a brown solid.

LCMS (ESI): m/z=577.67 [M+H]⁺.

### Preparation of 3-(6-(4-(1,3-dioxolan-2-yl)piperidin-1-yl)-1-methyl-1H-indazol-3-yl)piperidine-2,6-dione

To a solution of 6-(4-(1,3-dioxolan-2-yl)piperidin-1-yl)-3-(2,6-bis(benzyloxy)pyridin-3-yl)-1-methyl-1H-indazole (1.80 g, 3.12 mmol) in THF (90 mL) was added 20% Pd(OH)₂ on carbon (1.80 g). The reaction mixture was stirred under H₂ (60 psi) atmosphere at room temperature for 16 h. The reaction mixture was filtered through a celite bed and washed with THF:DCM (1:1) (1000 mL). The collected filtrate was concentrated under reduced pressure to afford 3-(6-(4-(1,3-dioxolan-2-yl)piperidin-1-yl)-1-methyl-1H-indazol-3-yl)piperidine-2,6-dione (1.40 g) as a brown solid which was used in the next step without further purification.

LC-MS (ESI): m/z=399.51 [M+H]⁺.

### Preparation of 1-(3-(2,6-dioxopiperidin-3-yl)-1-methyl-1H-indazol-6-yl)piperidine-4-carbaldehyde

A solution of 3-(6-(4-(1,3-dioxolan-2-yl)piperidin-1-yl)-1-methyl-1H-indazol-3-yl)piperidine-2,6-dione (0.90 g, 2.26 mmol) in HCOOH (9 mL) was stirred at 80° C. for 6 h. The reaction mixture was concentrated, co-distilled with DCM (3×40 mL), and dried to afford 1-(3-(2,6-dioxopiperidin-3-yl)-1-methyl-1H-indazol-6-yl)piperidine-4-carbaldehyde (0.78 g) as a brown solid which was used in the next step without further purification.

LC-MS (ESI): m/z=355.41 [M+H]⁺.

### Preparation of 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-3,9-diazaspiro[5.5]undecan-3-yl)methyl)piperidin-1-yl)-1-methyl-1H-indazol-3-yl)piperidine-2,6-dione

To a solution of 1-(3-(2,6-dioxopiperidin-3-yl)-1-methyl-1H-indazol-6-yl)piperidine-4-carbaldehyde (0.90 g, 2.54 mmol) and (S)-10-((5-chloro-2-(3,9-diazaspiro[5.5]undecan-3-yl)pyridin-4-yl)amino)-2-cyclopropyl-3,3-difluoro-7-methyl-1,2,3,4-tetrahydro-[1,4]oxazepino[2,3-c]quinolin-6(7H)-one (0.45 g, 0.76 mmol) in THF (18 mL) was added CH₃COONa (0.63 g, 7.63 mmol) and AcOH (0.9 mL). The resulting mixture was stirred at room temperature for 2 h. Then sodium triacetoxyborohydride (1.62 g, 7.63 mmol) was added at 0° C.

The reaction was stirred at room temperature for 6 h. The reaction mixture was quenched with water (100 mL) and extracted with ethyl acetate (2×100 mL). The combined organic layers were washed with brine, dried over anhydrous Na₂SO₄, filtered, and concentrated under reduced pressure to provide a crude product that was purified by prep-HPLC to afford 3-(6-(4-((9-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-3,9-diazaspiro[5.5]undecan-3-yl)methyl)piperidin-1-yl)-1-methyl-1H-indazol-3-yl)piperidine-2,6-dione (0.20 g) as a white solid.

LC-MS (ESI): m/z=923.48 [M+H]⁺.

¹H NMR (400 MHz, DMSO-d₆): δ 10.84 (s, 1H), 8.32 (s, 1H), 8.06 (s, 1H), 8.00 (s, 1H), 7.91 (s, 1H), 7.47-7.45 (m, 3H), 6.88 (d, J=10.00 Hz, 1H), 6.81 (s, 1H), 6.41 (br, 1H), 6.06 (s, 1H), 4.41-4.26 (m, 2H), 4.23-4.22 (m, 1H), 3.87 (s, 3H), 3.75 (d, J=12.00 Hz, 2H), 3.58 (s, 3H), 3.25-3.20 (m, 2H), 2.70-2.67 (m, 2H), 2.60-2.59 (m, 2H), 2.50-2.33 (m, 4H), 2.30-2.18 (m, 3H), 1.77 (d, J=12.40 Hz, 3H), 1.43-1.19 (m, 14H), 0.75-0.65 (m, 1H), 0.50-0.49 (m, 2H), 0.39-0.25 (m, 1H).

### Example 121: 3-[6-[[(6R, 7S)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione

### [3-(6-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](Compound 492a)

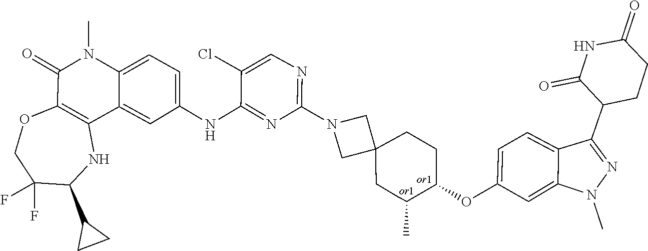 [SMILES: C[C@@H]1CCC2(CC[C@@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of 3-[6-[[(6R, 7S)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione [3-(6-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione]

To a solution of (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (100 mg, 213.55 mol, 1 equiv.) in DMSO (1 mL) was added 3-[1-methyl-6-[[(6R, 7S)-6-methyl-2-azaspiro[3.5]nonan-7-yl]oxy]indazol-3-yl]piperidine-2,6-dione (84.67 mg, 213.55 mol, 1.2 equiv.) and TEA (86.43 mg, 854.18 mol, 118.89 μL, 4 equiv.). The mixture was stirred at 100° C. for 2 hr. The reaction mixture was then filtered, and the filtrate was concentrated to give a residue which was purified by prep-HPLC to afford 3-[6-[[(6R, 7S)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione [3-(6-(((6R*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](28.99 mg) as a yellow solid.

LCMS (ESI): m/z=828.5 [M+H]⁺.

¹H NMR (400 MHz, DMSO-d₆) δ=10.86 (s, 1H), 8.75 (s, 1H), 8.26 (br s, 1H), 8.02 (s, 1H), 7.83 (br d, J=8.4 Hz, 1H), 7.56 (d, J=8.8 Hz, 1H), 7.44 (d, J=9.2 Hz, 1H), 7.06 (s, 1H), 6.76 (d, J=9.2 Hz, 1H), 6.11 (s, 1H), 4.52-4.34 (m, 2H), 4.40-4.32 (m, 1H), 4.29 (dd, J=5.2, 9.6 Hz, 1H), 3.91 (s, 3H), 3.73 (d, J=3.6 Hz, 2H), 3.65 (s, 2H), 3.57 (s, 3H), 2.65-2.59 (m, 3H), 2.20-2.12 (m, 1H), 1.99 (d, J=12.0 Hz, 1H), 1.89-1.61 (m, 5H), 1.60-1.39 (m, 2H), 1.36-1.21 (m, 1H), 0.98 (d, J=6.0 Hz, 3H), 0.72 (d, J=7.2 Hz, 1H), 0.53 (t, J=5.6 Hz, 2H), 0.42-0.30 (m, 1H)

### Example 122: 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (Compound 466b)

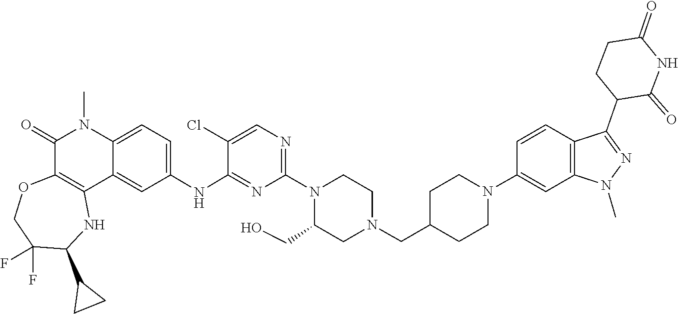 [SMILES: Cn1nc(C2CCC(=O)NC2=O)c2ccc(N3CCC(CN4CCN(c5ncc(Cl)c(Nc6ccc7c(c6)c6c(c(=O)n7C)OCC(F)(F)[C@H](C7CC7)N6)n5)[C@@H](CO)C4)CC3)cc21]

### Preparation of tert-butyl (R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazine-1-carboxylate

To a stirred solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.18 g, 0.38 mmol) and tert-butyl (R)-3-(hydroxymethyl)piperizine-1-carboxylate (0.50 g, 2.30 mmol) in DMSO (3.60 mL) was added DIPEA (1.80 mL). The reaction mixture was heated to 100° C. for 8 h. The reaction mixture was poured into ice cold water (30 mL), and the precipitated solid was filtered and dried under vacuum to obtain a crude product. The crude product was triturated with n-pentane (20 mL) and dried under vacuum to afford tert-butyl (R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazine-1-carboxylate (0.15 g) as a pale brown solid.

LCMS (ESI): m/z=648.59 [M+H]⁺

### Preparation of (S)-10-((5-chloro-2-((R)-2-(hydroxymethyl)piperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one

To a solution of tert-butyl (R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazine-1-carboxylate (0.125 g, 0.193 mmol, 1.0 equiv.) in DCM (2.500 mL) was added TFA (1.250 mL). The reaction mixture was stirred at room temperature for 2 h. The reaction mixture was concentrated under vacuum to obtain a crude product that was triturated with n-pentane (20 mL) and dried under vacuum to afford (S)-10-((5-chloro-2-((R)-2-(hydroxymethyl)piperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one (0.25 g) as a pale brown semi-solid.

LC-MS (ESI): m/z=548.43 [M+H]⁺.

### Preparation of 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione

A solution of 1-(3-(2,6-dioxopiperidin-3-yl)-1-methyl-1H-indazol-6-yl)piperidine-4-carbaldehyde (0.25 g, 0.39 mmol), (S)-10-((5-chloro-2-((R)-2-(hydroxymethyl)piperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one (0.21 g, 0.39 mmol), sodium acetate anhydrous (0.095 g, 1.16 mmol), and acetic acid (0.025 mL) in THF (10.0 mL) was stirred for 2 h. Sodium triacetoxyborohydride (0.25 g, 1.157 mmol) was then added, and the reaction mixture was stirred at room temperature for 2 h. The reaction mixture was diluted with ethyl acetate (50 mL) and quenched with sat. NH₄Cl solution (20 mL). The separated organic layer was dried over anhydrous Na₂SO₄, filtered, and dried under vacuum to obtain a crude product that was purified by prep-HPLC to afford 3-(6-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (0.04 g) as an off white solid.

LC-MS (ESI): m/z=884.37 [M−H]⁻

¹H NMR (400 MHz, DMSO-d₆): δ 10.89 (s, 1H), 8.81 (s, 1H), 8.22 (s, 1H), 8.04 (s, 1H), 7.71 (d, J=8.40 Hz, 1H), 7.49 (d, J=9.20 Hz, 1H), 7.47 (d, J=9.20 Hz, 1H), 6.89-6.87 (m, 1H), 6.82 (s, 1H), 6.27 (br s, 1H), 4.51-4.48 (m, 1H), 4.24-4.23 (m, 5H), 3.88 (s, 3H), 3.77-3.72 (m, 3H), 3.55 (s, 3H), 3.03 (d, J=10.80 Hz, 3H), 2.67-2.67 (m, 2H), 2.61-2.57 (m, 4H), 2.31-2.28 (m, 1H), 2.15-2.13 (m, 3H), 1.82-1.75 (m, 8H), 1.70 (s, 3H), 1.36 (s, 3H), 1.26-1.23 (m, 3H), 0.72-0.71 (m, 1H), 0.52-0.51 (m, 2H), 0.32 (d, J=4.00 Hz, 1H).

### Example 123: 3-(6-(4-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 486b)

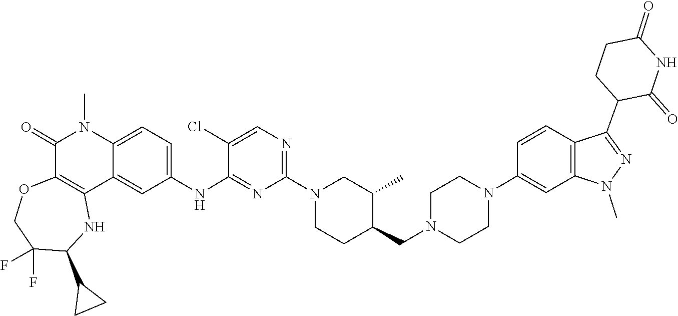 [SMILES: C[C@H]1CN(c2ncc(Cl)c(Nc3ccc4c(c3)c3c(c(=O)n4C)OCC(F)(F)[C@H](C4CC4)N3)n2)CC[C@@H]1CN1CCN(c2ccc3c(C4CCC(=O)NC4=O)nn(C)c3c2)CC1]

### Preparation of tert-butyl (3R, 4S)-4-(4-(3-(2,6-bis(benzyloxy)pyridin-3-yl)-1-methyl-1H-indazol-6-yl)piperazine-1-carbonyl)-3-methylpiperidine-1-carboxylate

To a solution of (3R, 4S)-1-(tert-butoxycarbonyl)-3-methylpiperidine-4-carboxylic acid (0.45 g, 1.85 mmol) and 3-(2,6-bis(benzyloxy)pyridin-3-yl)-1-methyl-6-(piperazin-1-yl)-1H-indazole (0.93 g, 1.85 mmol) in DMF (4.5 mL) was added DIPEA (2.5 mL, 14.8 mmol) and HATU (1.0 g, 2.77 mmol). The reaction mixture was stirred at rt for 2 h. After completion, the reaction was diluted with ethyl acetate (50 mL) and washed with cold water (3×20 mL). The organic layer was dried over anhydrous sodium sulfate and concentrated under reduced pressure to provide a crude product that was triturated by using n-pentane to afford tert-butyl (3R, 4S)-4-(4-(3-(2,6-bis(benzyloxy)pyridin-3-yl)-1-methyl-1H-indazol-6-yl)piperazine-1-carbonyl)-3-methylpiperidine-1-carboxylate (1.20 g) as an off white solid.

LC-MS (ESI): m/z=731.85 [M+H]⁺.

### Preparation of tert-butyl (3R, 4S)-4-((4-(3-(2,6-bis(benzyloxy)pyridin-3-yl)-1-methyl-1H-indazol-6-yl)piperazin-1-yl)methyl)-3-methylpiperidine-1-carboxylate

To a solution of tert-butyl (3R, 4S)-4-(4-(3-(2,6-bis(benzyloxy)pyridin-3-yl)-1-methyl-1H-indazol-6-yl)piperazine-1-carbonyl)-3-methylpiperidine-1-carboxylate (1.2 g, 1.64 mmol) in THF (24 mL) was added borane dimethylsulfide solution in THF (2.0 M, 24 mL). The reaction mixture was stirred at rt for 8 h. After completion, the reaction mixture was quenched with methanol and evaporated under reduced pressure. Then the residue was dissolved in methanol and refluxed at 80° C. for 2 h. The reaction mixture was concentrated under reduced pressure to provide a crude product that was purified by flash column chromatography (SiO₂, 100-200 mesh, 50% ethyl acetate in petroleum ether) to afford tert-butyl (3R, 4S)-4-((4-(3-(2,6-bis(benzyloxy)pyridin-3-yl)-1-methyl-1H-indazol-6-yl)piperazin-1-yl)methyl)-3-methylpiperidine-1-carboxylate (0.8 g) as a brown semi-solid.

LC-MS (ESI): m/z=717.5 [M+H]⁺.

### Preparation of tert-butyl (3R, 4S)-4-((4-(3-(2,6-dioxopiperidin-3-yl)-1-methyl-1H-indazol-6-yl)piperazin-1-yl)methyl)-3-methylpiperidine-1-carboxylate

To a solution of tert-butyl (3R, 4S)-4-((4-(3-(2,6-bis(benzyloxy)pyridin-3-yl)-1-methyl-1H-indazol-6-yl)piperazin-1-yl)methyl)-3-methylpiperidine-1-carboxylate (0.40 g, 0.55 mmol) in THF (12.0 mL) was added 20% Pd(OH)₂ on carbon (0.40 g, 100% w/w). The reaction mixture was stirred under hydrogen atmosphere (80 psi) at room temperature for 16 h. The reaction mixture was diluted with ethyl acetate (50 mL), filtered through a celite bed, and washed with 50% THF in ethyl acetate (200 mL). The collected filtrate was concentrated under reduced pressure to provide a crude product that was triturated with diethyl ether to afford tert-butyl (3R, 4S)-4-((4-(3-(2,6-dioxopiperidin-3-yl)-1-methyl-1H-indazol-6-yl)piperazin-1-yl)methyl)-3-methylpiperidine-1-carboxylate (0.30 g) as a brown semi-solid.

LC-MS (ESI): m/z=539.61 [M+H]⁺

### Preparation of 3-(1-methyl-6-(4-(((3R, 4S)-3-methylpiperidin-4-yl)methyl)piperazin-1-yl)-1H-indazol-3-yl)piperidine-2,6-dione

To a solution of tert-butyl (3R, 4S)-4-((4-(3-(2,6-dioxopiperidin-3-yl)-1-methyl-1H-indazol-6-yl)piperazin-1-yl)methyl)-3-methylpiperidine-1-carboxylate (0.30 g, 0.56 mmol) in DCM (3.0 mL) was added TFA (1.5 mL). The reaction mixture was stirred at room temperature for 1 h. The reaction mixture was concentrated and co-distilled with petroleum ether (2×10) under reduced pressure to provide a crude product that was triturated with diethyl ether to afford 3-(1-methyl-6-(4-(((3R, 4S)-3-methylpiperidin-4-yl)methyl)piperazin-1-yl)-1H-indazol-3-yl)piperidine-2,6-dione (0.24 g) as an off white solid.

LC-MS (ESI): m/z=439.45 [M+H]⁺.

### Preparation of 3-(6-(4-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-6-(4-(((3R, 4S)-3-methylpiperidin-4-yl)methyl)piperazin-1-yl)-1H-indazol-3-yl)piperidine-2,6-dione (0.1 g, 0.23 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.03 g, 0.07 mmol) in DMSO (2 mL) was added DIPEA (0.2 mL, 1.15 mmol). The resulting reaction mixture was stirred at 100° C. for 6 h. The reaction mixture was poured into ice-cold water and stirred for 15 minutes, and the resulting precipitate was filtered and dried to get crude product that was purified by prep-HPLC to afford 3-(6-(4-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (36 mg) as a white solid.

LC-MS (ESI): m/z=868.44 [M+H]⁺.

¹H NMR (400 MHz, DMSO-d6): δ 10.84 (s, 1H), 8.77 (s, 1H), 8.38 (s, 1H), 8.16 (s, 1H), 8.02 (s, 1H), 7.75 (d, J=8.80 Hz, 1H), 7.47 (dd, J=9.20, 20.40 Hz, 2H), 6.91 (d, J=8.80 Hz, 1H), 6.82 (s, 1H), 6.20 (s, 1H), 4.26-4.25 (m, 5H), 3.88 (s, 3H), 3.56 (s, 3H), 3.32-3.20 (m, 5H), 2.80-2.79 (m, 1H), 2.64-2.62 (m, 4H), 2.50-2.42 (m, 3H), 2.30-2.29 (m, 1H), 2.14-2.12 (m, 2H), 1.91-1.90 (m, 1H), 1.50-1.20 (m, 3H), 1.16-0.99 (m, 1H), 0.87 (d, J=6 Hz, 3H), 0.71 (d, J=7.20 Hz, 1H), 0.51 (t, J=5.60 Hz, 2H), 0.35 (d, J=5.20 Hz, 1H).

### Example 124: 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 486a)

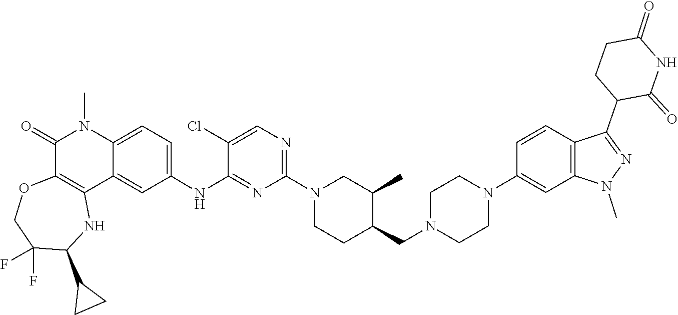 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCN(c2ccc3c(C4CCC(=O)NC4=O)nn(C)c3c2)CC1]

### Preparation of 3-(2,6-bis(benzyloxy)pyridin-3-yl)-1-methyl-6-(piperazin-1-yl)-1H-indazole

To a solution of 3-(2,6-bis(benzyloxy)pyridin-3-yl)-6-bromo-1-methyl-1H-indazole (1.0 g, 1.99 mmol), tert-butyl piperazine-1-carboxylate (0.17 g, 1.99 mmol), and cesium carbonate (1.95 g, 5.99 mmol) in dioxane (20.0 mL) was added Ruphos (0.09 g, 0.20 mmol) and RuPhos Pd G3 (0.08 g, 0.10 mmol) under argon. The reaction mixture was stirred and heated to 100° C. for 16 h. The reaction mixture was filtered through a pad of celite and washed with ethyl acetate (200 mL). The collected filtrate was concentrated under reduced pressure to provide a crude product that was purified by flash column chromatography (silica 100-200 mesh, 15-20% ethyl acetate in petroleum ether as eluent) to afford 3-(2,6-bis(benzyloxy)pyridin-3-yl)-1-methyl-6-(piperazin-1-yl)-1H-indazole (0.51 g) as a pale brown solid.

LC-MS (ESI): m/z=606.75 [M+H]⁺

### Preparation of tert-butyl (3S, 4S)-4-(4-(3-(2,6-bis(benzyloxy)pyridin-3-yl)-1-methyl-1H-indazol-6-yl) piperazine-1-carbonyl)-3-methylpiperidine-1-carboxylate

To a solution of 3-(2,6-bis(benzyloxy)pyridin-3-yl)-1-methyl-6-(piperazin-1-yl)-1H-indazole (0.45 g, 0.02 mmol), (3S, 4S)-1-(tert-butoxycarbonyl)-3-methylpiperidine-4-carboxylic acid (0.22 g, 0.02 mmol), and DIPEA (1.10 mL, 6.23 mmol) in DMF (4.50 mL) was added HATU (0.50 g, 1.33 mmol). The reaction mixture was stirred at room temperature for 1 h. The reaction mixture was diluted with water (10 mL), and the precipitated solid was filtered and dried under reduced pressure to obtain crude product that was triturated with diethyl ether (20 mL) and dried under vacuum to afford tert-butyl (3S, 4S)-4-(4-(3-(2,6-bis(benzyloxy)pyridin-3-yl)-1-methyl-1H-indazol-6-yl) piperazine-1-carbonyl)-3-methylpiperidine-1-carboxylate (0.51 g) as an off-white solid.

LC-MS (ESI): m/z=731.46 [M+H]⁺

### Preparation of tert-butyl (3S, 4S)-4-((4-(3-(2,6-bis(benzyloxy)pyridin-3-yl)-1-methyl-1H-indazol-6-yl) piperazin-1-yl) methyl)-3-methylpiperidine-1-carboxylate

To a solution of tert-butyl (3S, 4S)-4-(4-(3-(2,6-bis(benzyloxy)pyridin-3-yl)-1-methyl-1H-indazol-6-yl) piperazine-1-carbonyl)-3-methylpiperidine-1-carboxylate (0.45 g, 0.02 mmol) in THF (5.0 mL) was added BH₃ in DMS (neat, 2.5 mL) at 0° C. The reaction mixture was stirred at room temperature for 7 h. The reaction mixture was quenched with methanol (100 mL) and concentrated under reduced pressure to provide a crude product that was purified by flash column chromatography (SiO₂, 230-400 mesh; 20% EtOAc in petroleum ether as eluent) to afford tert-butyl (3S, 4S)-4-((4-(3-(2,6-bis(benzyloxy)pyridin-3-yl)-1-methyl-1H-indazol-6-yl) piperazin-1-yl) methyl)-3-methylpiperidine-1-carboxylate (0.45 g) as an off white solid.

LC-MS (ESI): m/z=717.53 [M+H]⁺

### Preparation of tert-butyl (3S, 4S)-4-((4-(3-(2,6-dioxopiperidin-3-yl)-1-methyl-1H-indazol-6-yl) piperazin-1-yl) methyl)-3-methylpiperidine-1-carboxylate

To a solution of tert-butyl (3S, 4S)-4-((4-(3-(2,6-bis(benzyloxy)pyridin-3-yl)-1-methyl-1H-indazol-6-yl) piperazin-1-yl) methyl)-3-methylpiperidine-1-carboxylate (0.36 g, 0.50 mmol) in THF (108.0 mL) was added 20% Pd(OH)₂ on carbon (1.08 g). The reaction mixture was stirred under hydrogen atmosphere (80 psi) at room temperature for 18 h. The reaction was diluted with THF (200 mL) and filtered through a pad of celite and washed with excess of 40% THF in DCM (800 mL). The collected filtrate was concentrated under vacuum to provide a crude product that was triturated with n-pentane (50 mL) and dried under vacuum to afford tert-butyl (3S, 4S)-4-((4-(3-(2,6-dioxopiperidin-3-yl)-1-methyl-1H-indazol-6-yl) piperazin-1-yl) methyl)-3-methylpiperidine-1-carboxylate (0.22 g) as a pale brown semi solid.

LC-MS (ESI): m/z=539.67 [M+H]⁺

### Preparation of 3-(1-methyl-6-(4-(((3S, 4S)-3-methylpiperidin-4-yl) methyl) piperazin-1-yl)-1H-indazol-3-yl) piperidine-2,6-dione

To a solution of tert-butyl (3S, 4S)-4-((4-(3-(2,6-dioxopiperidin-3-yl)-1-methyl-1H-indazol-6-yl) piperazin-1-yl) methyl)-3-methylpiperidine-1-carboxylate (0.22 g, 0.41 mmol) in DCM (6.60 mL) was added TFA (2.20 mL). The reaction mixture was stirred at room temperature for 2 h. The reaction mixture was concentrated under vacuum to provide a crude product that was triturated with DEE (10 mL). The precipitated solid was filtered and concentrated under vacuum to afford 3-(1-methyl-6-(4-(((3S, 4S)-3-methylpiperidin-4-yl) methyl) piperazin-1-yl)-1H-indazol-3-yl) piperidine-2,6-dione (0.17 g, TFA salt) as a pale brown semi solid.

LC-MS (ESI): m/z=439.61 [M+H]⁺

### Preparation of 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-6-(4-(((3S, 4S)-3-methylpiperidin-4-yl) methyl) piperazin-1-yl)-1H-indazol-3-yl) piperidine-2,6-dione (0.25 g, 0.57 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.053 g, 0.11 mmol) in DMSO (5.0 mL) was added DIPEA (2.50 mL). The reaction mixture was heated to 100° C. for 18 h. The reaction mixture was poured into ice-cold water (20 mL), and the precipitated solid was filtered and dried under vacuum to provide a crude product that was purified by prep-HPLC to afford 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (0.039 g) as an off-white solid.

LC-MS (ESI): m/z=870.53 [M+H]⁺ 1H-NMR (400 MHz, DMSO-d6): δ 10.84 (s, 1H), 8.72 (s, 1H), 8.14 (s, 1H), 8.00 (s, 1H), 7.77 (dd, J=1.60, 9.00 Hz, 1H), 7.46-7.44 (m, 2H), 6.91 (d, J=9.20 Hz, 1H), 6.83 (s, 1H), 6.18 (s, 1H), 4.36-4.27 (m, 3H), 4.25-4.24 (m, 2H), 3.89 (s, 3H), 3.56 (s, 3H), 3.20 (br s, 5H), 3.02 (d, J=11.60 Hz, 1H), 2.62-2.61 (m, 1H), 2.59-2.51 (m, 6H), 2.32-2.31 (m, 1H), 2.17-2.15 (m, 3H), 1.92-1.90 (m, 2H), 1.34-1.33 (m, 4H), 0.72-0.70 (m, 3H), 0.52-0.50 (m, 2H), 0.35 (d, J=6.00 Hz, 1H).

### Example 125: 3-(7-(4-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,6-diazabicyclo[3.1.1]heptan-6-yl)methyl)piperidin-1-yl)-1-methyl-1H-indazol-3-yl)piperidine-2,6-dione (Compound 488a)

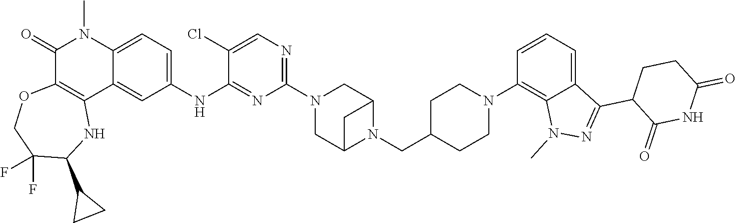 [SMILES: Cn1nc(C2CCC(=O)NC2=O)c2cccc(N3CCC(CN4C5CC4CN(c4ncc(Cl)c(Nc6ccc7c(c6)c6c(c(=O)n7C)OCC(F)(F)[C@H](C7CC7)N6)n4)C5)CC3)c21]

### Preparation of tert-butyl 3-((1-((benzyloxy)carbonyl)piperidin-4-yl)methyl)-3,6-diazabicyclo[3.1.1]heptane-6-carboxylate

To a solution of tert-butyl 3,6-diazabicyclo [3.1.1]heptane-3-carboxylate (2.0 g, 10.08 mmol), benzyl 4-formylpiperidine-1-carboxylate (2.99 g, 12.10 mmol), acetic acid (2.0 mL) in DCM (40 mL) was added sodium triacetoxyborohydride (2.13 g, 10.08 mmol) at 0° C. under nitrogen atmosphere. The reaction mixture was stirred at room temperature for 16 h. The reaction mixture was diluted with water (50 mL) and extracted with ethyl acetate (2×100 mL). The combined organic layers were washed with brine (20 mL), dried over anhydrous Na₂SO₄, filtered, and concentrated under reduced pressure to provide a crude product that was purified by flash column chromatography (SiO₂, 60-120 mesh, 25% ethyl acetate in petroleum ether as eluent) to afford tert-butyl 3-((1-((benzyloxy)carbonyl)piperidin-4-yl)methyl)-3,6-diazabicyclo[3.1.1]heptane-6-carboxylate (2.3 g) as a yellow semi-solid.

LC-MS (ESI): m/z=430.32 [M+H]⁺

### Preparation of tert-butyl 6-(piperidin-4-ylmethyl)-3,6-diazabicyclo[3.1.1]heptane-3-carboxylate

To a solution of tert-butyl 3-((1-((benzyloxy)carbonyl)piperidin-4-yl)methyl)-3,6-diazabicyclo[3.1.1]heptane-6-carboxylate (2.3 g, 5.35 mmol) in THF (69 mL) was added 20% Pd(OH)₂ on carbon (2.3 g, 100% w/w). The reaction mixture was stirred under hydrogen atmosphere (80 psi) at room temperature for 16 h. The reaction mixture was diluted with DCM (50 mL) and filtered through a celite bed and washed with 30% THF in DCM (500 mL). The collected filtrate was concentrated under reduced pressure and co-distilled with toluene (20 mL) to afford tert-butyl 6-(piperidin-4-ylmethyl)-3,6-diazabicyclo[3.1.1]heptane-3-carboxylate (1.1 g) as an off white solid.

LC-MS (ESI): m/z=296.56 [M+H]⁺

### Preparation of tert-butyl 6-((1-(3-(2,6-bis(benzyloxy)pyridin-3-yl)-1-methyl-1H-indazol-7-yl)piperidin-4-yl)methyl)-3,6-diazabicyclo[3.1.1]heptane-3-carboxylate

To a solution of 3-(2,6-bis(benzyloxy)pyridin-3-yl)-7-bromo-1-methyl-1H-indazole (1.0 g, 1.99 mmol), tert-butyl 6-(piperidin-4-ylmethyl)-3,6-diazabicyclo[3.1.1]heptane-3-carboxylate (0.93 g, 2.99 mmol), and cesium carbonate (1.94 g, 5.97 mmol) in 1,4-dioxane (18 mL) was added Pd-PEPPSI-IHeptCl (0.09 g, 0.09 mmol) under argon. The mixture was then stirred and heated to 100° C. for 16 h. The reaction mixture was diluted with EtOAc (20 mL), filtered through a celite bed, and washed with ethyl acetate (200 mL). The collected filtrate was concentrated under reduced pressure to provide a crude product that was purified by flash column chromatography (SiO₂, 100-200 mesh, 50% ethyl acetate in petroleum ether as eluent) to afford tert-butyl 6-((1-(3-(2,6-bis(benzyloxy)pyridin-3-yl)-1-methyl-1H-indazol-7-yl)piperidin-4-yl)methyl)-3,6-diazabicyclo[3.1.1]heptane-3-carboxylate (1.0 g) as a brown semi-solid.

LC-MS (ESI): m/z=715.77 [M+H]⁺.

### Preparation of tert-butyl 6-((1-(3-(2,6-dioxopiperidin-3-yl)-1-methyl-1H-indazol-7-yl)piperidin-4-yl)methyl)-3,6-diazabicyclo[3.1.1]heptane-3-carboxylate

To a solution of tert-butyl 6-((1-(3-(2,6-bis(benzyloxy)pyridin-3-yl)-1-methyl-1H-indazol-7-yl)piperidin-4-yl)methyl)-3,6-diazabicyclo[3.1.1]heptane-3-carboxylate (1.0 g, 1.39 mmol) in THF (30.0 mL) was added 20% Pd (OH)₂ on carbon (1.0 g). The reaction mixture was stirred under hydrogen atmosphere (80 Psi) at room temperature for 18 h. The reaction mixture was diluted with DCM (50 mL), filtered through a celite bed, and washed with 30% THF in DCM (200 mL).

The collected filtrate was concentrated under reduced pressure to provide a crude product that was triturated with diethyl ether (20 mL) to afford tert-butyl 6-((1-(3-(2,6-dioxopiperidin-3-yl)-1-methyl-1H-indazol-7-yl)piperidin-4-yl)methyl)-3,6-diazabicyclo[3.1.1]heptane-3-carboxylate (0.81 g) as an off white solid.

LC-MS (EIS): m/z=537.41 [M+H]⁺

### Preparation of 3-(7-(4-((3,6-diazabicyclo[3.1.1]heptan-3-yl)methyl)piperidin-1-yl)-1-methyl-1H-indazol-3-yl)piperidine-2,6-dione

To a solution of tert-butyl 6-((1-(3-(2,6-dioxopiperidin-3-yl)-1-methyl-1H-indazol-7-yl)piperidin-4-yl)methyl)-3,6-diazabicyclo[3.1.1]heptane-3-carboxylate (0.81 g, 1.50 mmol) in DCM (4.0 mL) was added TFA (4.0 mL). The reaction mixture was stirred at room temperature for 1 h. The reaction mixture was concentrated and co-distilled with petroleum ether (30 mL) under reduced pressure to provide a crude product that was triturated with diethyl ether (20 mL) to afford 3-(7-(4-((3,6-diazabicyclo[3.1.1]heptan-3-yl)methyl)piperidin-1-yl)-1-methyl-1H-indazol-3-yl)piperidine-2,6-dione (0.80 g) as a pale brown semi solid.

LC-MS (ESI): m/z=437.54 [M+H]⁺.

### Preparation of 3-(7-(4-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,6-diazabicyclo[3.1.1]heptan-6-yl)methyl)piperidin-1-yl)-1-methyl-1H-indazol-3-yl)piperidine-2,6-dione

A stirred solution of 3-(7-(4-((3,6-diazabicyclo[3.1.1]heptan-3-yl)methyl)piperidin-1-yl)-1-methyl-1H-indazol-3-yl)piperidine-2,6-dione (0.40 g, 0.91 mmol), (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.12 g, 0.27 mmol), and N, N-diisopropylethylamine (0.58 g, 4.55 mmol) in DMSO (8.0 mL) was heated at 100° C. for 16 h. The reaction mixture was poured into ice-cold water (20 mL), and the precipitated solid was filtered and dried to obtain crude product that was purified by prep-HPLC to afford 3-(7-(4-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,6-diazabicyclo[3.1.1]heptan-6-yl)methyl)piperidin-1-yl)-1-methyl-1H-indazol-3-yl)piperidine-2,6-dione (0.0052 g) as an off-white solid.

¹H NMR (400 MHz, DMSO-d6): δ 10.85 (s, 1H), 8.81 (s, 1H), 8.35 (s, 1H), 8.11 (s, 1H), 7.87-7.81 (m, 1H), 7.43 (d, J=8.80 Hz, 1H), 7.35 (d, J=7.60 Hz, 1H), 7.01-6.95 (m, 2H), 6.23 (s, 1H), 4.45-4.41 (m, 3H), 4.20 (d, J=2.80 Hz, 3H), 3.71-3.68 (m, 6H), 3.21-3.15 (m, 4H), 2.61-2.58 (m, 4H), 2.50-2.50 (m, 3H), 2.30-2.26 (m, 2H), 2.18-2.14 (m, 1H), 1.86-1.75 (m, 2H), 1.46-1.42 (m, 5H), 1.35-1.24 (m, 1H), 0.82-0.79 (m, 1H), 0.50-0.48 (m, 2H), 0.34-0.34 (m, 1H).

### Example 126: 3-(7-((1S, 4S)-5-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2,5-diazabicyclo[2.2.1]heptan-2-yl)-1-methyl-1H-indazol-3-yl)piperidine-2,6-dione (Compound 482b)

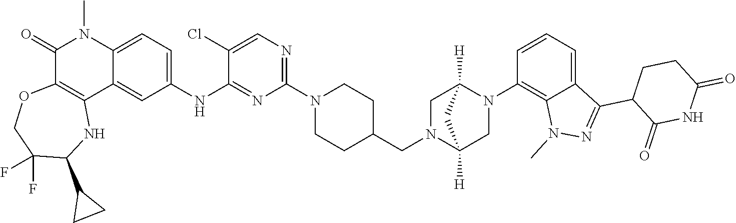 [SMILES: Cn1nc(C2CCC(=O)NC2=O)c2cccc(N3C[C@@H]4C[C@H]3CN4CC3CCN(c4ncc(Cl)c(Nc5ccc6c(c5)c5c(c(=O)n6C)OCC(F)(F)[C@H](C6CC6)N5)n4)CC3)c21]

### Preparation of tert-butyl (1S, 4S)-5-(3-(2,6-bis(benzyloxy)pyridin-3-yl)-1-methyl-1H-indazol-7-yl)-2,5-diazabicyclo[2.2.1]heptane-2-carboxylate

To a solution of 3-(2,6-bis(benzyloxy)pyridin-3-yl)-7-bromo-1-methyl-1H-indazole (1.2 g, 2.40 mmol) and tert-butyl (1R, 4R)-2,5-diazabicyclo[2.2.1]heptane-2-carboxylate (0.57 g, 2.88 mmol) in 1,4-dioxane (24 mL) was added cesium carbonate (2.34 g, 7.20 mmol) and Pd-PEPPSI-iHeptCl (0.11 g, 0.12 mmol) under argon. The reaction mixture was stirred at 100° C. for 12 h.

The reaction mixture was then cooled to room temperature, filtered through a celite bed, and washed with ethyl acetate (100 mL). The filtrate was concentrated to provide a crude product that was purified by flash column chromatography (SiO₂, 230-400 mesh, 15% ethyl acetate in petroleum ether) to afford tert-butyl (1S, 4S)-5-(3-(2,6-bis(benzyloxy)pyridin-3-yl)-1-methyl-1H-indazol-7-yl)-2,5-diazabicyclo[2.2.1]heptane-2-carboxylate (1.0 g) as a pale yellow solid.

LC-MS (ESI): 618.35 [M+H]⁺

### Preparation of 3-(7-((1S, 4S)-2,5-diazabicyclo[2.2.1]heptan-2-yl)-1-methyl-1H-indazol-3-yl)-6-(benzyloxy)pyridin-2-ol

To a solution of tert-butyl (1S, 4S)-5-(3-(2,6-bis(benzyloxy)pyridin-3-yl)-1-methyl-1H-indazol-7-yl)-2,5-diazabicyclo[2.2.1]heptane-2-carboxylate (1.0 g, 1.62 mmol)) in DCM (20.0 mL) was added TFA (5.0 mL) at 0° C. The reaction mixture was stirred at room temperature for 2 h. The reaction mixture was concentrated under reduced pressure and co-distilled with diethyl ether to provide a crude product that was washed with n-pentane to obtain 3-(7-((1S, 4S)-2,5-diazabicyclo[2.2.1]heptan-2-yl)-1-methyl-1H-indazol-3-yl)-6-(benzyloxy)pyridin-2-ol (0.60 g) as semi-solid.

LC-MS (ESI): m/z=428.51 [M+H]⁺.

### Preparation of benzyl 4-(((1S, 4S)-5-(3-(6-(benzyloxy)-2-hydroxy pyridin-3-yl)-1-methyl-1H-indazol-7-yl)-2,5-diazabicyclo[2.2.1]heptan-2-yl)methyl)piperidine-1-carboxylate

To a solution of 3-(7-((1S, 4S)-2,5-diazabicyclo[2.2.1]heptan-2-yl)-1-methyl-1H-indazol-3-yl)-6-(benzyloxy)pyridin-2-ol (0.60 g, 1.40 mmol) and benzyl 4-formylpiperidine-1-carboxylate (0.41 g, 1.68 mmol) in THF (30 mL) was added acetic acid (0.6 mL) and sodium acetate (0.34 g, 2.79 mmol) at 0° C. The reaction mixture was stirred for 2 h. NaBH(OAc)₃ (0.88 g, 2.79 mmol) was then added at 0° C., and the reaction mixture was stirred at room temperature for 16 h. The reaction mixture was quenched with ice-cold water (100 mL) and extracted with DCM (3×100 mL). The combined organic layers were washed with brine (100 mL), dried over anhydrous Na₂SO₄, filtered and dried under vacuum to provide a crude product that was purified by flash column chromatography (SiO₂, 230-400, 10% methanol in DCM) to afford benzyl 4-(((1S, 4S)-5-(3-(6-(benzyloxy)-2-hydroxy pyridin-3-yl)-1-methyl-1H-indazol-7-yl)-2,5-diazabicyclo[2.2.1]heptan-2-yl)methyl)piperidine-1-carboxylate (1.0 g) as a brown semi solid.

LC-MS (ESI): m/z=659.64 [M+H]⁺

### Preparation of 3-(1-methyl-7-((1S, 4S)-5-(piperidin-4-ylmethyl)-2,5-diazabicyclo[2.2.1]heptan-2-yl)-1H-indazol-3-yl)piperidine-2,6-dione

To a solution of benzyl 4-(((1S, 4S)-5-(3-(6-(benzyloxy)-2-hydroxy pyridin-3-yl)-1-methyl-1H-indazol-7-yl)-2,5-diazabicyclo[2.2.1]heptan-2-yl)methyl)piperidine-1-carboxylate (1.0 g, 1.51 mmol) in THF (20.0 mL) was added 20% Pd(OH)₂ on carbon (1.2 g, 100% w/w). The reaction mixture was stirred under hydrogen atmosphere (80 psi) for 12 h at room temperature. The reaction mixture was diluted with THF and filtered through a pad of celite and washed with excess of 20% THF in DCM. The filtrate was collected and concentrated to provide 3-(1-methyl-7-((1S, 4S)-5-(piperidin-4-ylmethyl)-2,5-diazabicyclo[2.2.1]heptan-2-yl)-1H-indazol-3-yl)piperidine-2,6-dione as a brown semi-solid (0.52 g) which was used in the next step without further purification.

LC-MS (ESI): m/z=437.50 [M+H]⁺

### Preparation of 3-(7-((1S, 4S)-5-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2,5-diazabicyclo[2.2.1]heptan-2-yl)-1-methyl-1H-indazol-3-yl)piperidine-2,6-dione

To a solution of 3-(1-methyl-7-((1S, 4S)-5-(piperidin-4-ylmethyl)-2,5-diazabicyclo[2.2.1]heptan-2-yl)-1H-indazol-3-yl)piperidine-2,6-dione (0.20 g, 0.45 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinoline-6(7H)-one (0.08 g, 0.18 mmol) in DMSO (4.0 mL) was added N,N-diisopropylethylamine (0.78 mL, 4.50 mmol). The resulting reaction mixture was stirred at 100° C. for 6 h. The reaction mixture was quenched with ice-cold water (50 mL), and the solid precipitated was filtered and dried under vacuum to provide a crude product that was purified by prep-HPLC. After purification, pure fractions were collected and concentrated to afford 3-(7-((1S, 4S)-5-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2,5-diazabicyclo[2.2.1]heptan-2-yl)-1-methyl-1H-indazol-3-yl)piperidine-2,6-dione (46 mg) as an off white solid.

LC-MS (ESI): 868.52 [M+H]⁺

¹H NMR (400 MHz, DMSO-d6): δ 10.90 (s, 1H), 8.77 (s, 1H), 8.32 (s, 1H), 8.18 (s, 1H), 8.02 (s, 1H), 7.74 (d, J=8.00 Hz, 1H), 7.44 (d, J=9.20 Hz, 1H), 7.29 (d, J=7.60 Hz, 1H), 6.99-6.97 (m, 2H), 6.02 (s, 1H), 4.31-4.30 (m, 5H), 4.21 (s, 3H), 3.88 (s, 1H), 3.57 (s, 3H), 3.55 (br, 2H), 3.22 (br, 1H), 3.05 (t, J=8.40 Hz, 1H), 2.77 (d, J=9.20 Hz, 2H), 2.61-2.59 (m, 4H), 2.37-2.33 (m, 3H), 1.78-1.75 (m, 4H), 1.60 (s, 1H), 1.31 (s, 1H), 1.04 (s, 2H), 0.69 (s, 1H), 0.51 (t, J=6.00 Hz, 2H), 0.35 (d, J=4.80 Hz, 1H).

### Example 127: 3-(6-(((6R, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](Compound 464d)

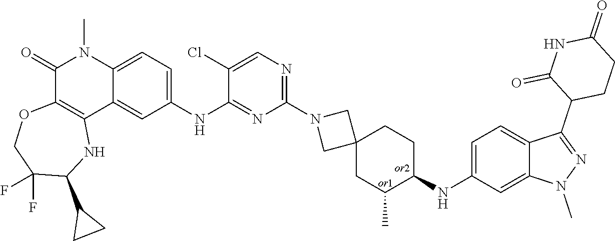 [SMILES: C[C@@H]1CC2(CC[C@H]1Nc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of tert-butyl (6R, 7R)-7-((3-(2,6-dioxopiperidin-3-yl)-1-methyl-1H-indazol-6-yl)amino)-6-methyl-2-azaspiro[3.5]nonane-2-carboxylate

To a solution of tert-butyl (6R, 7R)-7-((3-(2,6-bis(benzyloxy)pyridin-3-yl)-1-methyl-1H-indazol-6-yl)amino)-6-methyl-2-azaspiro[3.5]nonane-2-carboxylate (0.20 g, 0.29 mmol) in THF (8.0 mL) was added 20% Pd(OH)₂ on carbon (0.20 g) under N₂. The reaction mixture was stirred under H₂ (80 psi) pressure at room temperature for 16 h. The reaction mixture was filtered through a celite bed, concentrated under reduced pressure, washed with n-pentane, and dried to afford tert-butyl (6R, 7R)-7-((3-(2,6-dioxopiperidin-3-yl)-1-methyl-1H-indazol-6-yl)amino)-6-methyl-2-azaspiro[3.5]nonane-2-carboxylate (0.14 g) as a brown solid.

LC-MS (ESI): m/z=496.60 [M+H]⁺.

### Preparation of 3-(1-methyl-6-(((6R, 7R)-6-methyl-2-azaspiro[3.5]nonan-7-yl)amino)-1H-indazol-3-yl)piperidine-2,6-dione

To a solution of tert-butyl (6R, 7R)-7-((3-(2,6-dioxopiperidin-3-yl)-1-methyl-1H-indazol-6-yl)amino)-6-methyl-2-azaspiro[3.5]nonane-2-carboxylate (0.14 g, 1.28 mmol)) in DCM (3.0 mL) was cooled to 0° C. TFA (1.40 mL) was added, and the reaction mixture was stirred at room temperature for 2 h. The reaction mixture was concentrated under reduced pressure and washed with n-pentane to provide 3-(1-methyl-6-(((6R, 7R)-6-methyl-2-azaspiro[3.5]nonan-7-yl)amino)-1H-indazol-3-yl)piperidine-2,6-dione (0.2 g) as a brown solid.

LC-MS (ESI): m/z=396.46 [M+H]⁺

### Preparation of 3-(6-(((6R, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione

### [3-(6-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione]

To a solution of 3-(1-methyl-6-(((6R, 7R)-6-methyl-2-azaspiro[3.5]nonan-7-yl)amino)-1H-indazol-3-yl)piperidine-2,6-dione (0.10 g, 0.20 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.05 g, 0.12 mmol) in DMSO (2.0 mL) was added N,N-diisopropylethylamine (0.21 g, 1.62 mmol). The reaction mixture was stirred at 100° C. for 12 h.

The reaction mixture was poured into ice cold water and stirred for 15 minutes. The resulting precipitate was filtered, dried, and purified by prep-HPLC to afford 3-(6-(((6R, 7R)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione [3-(6-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)amino)-1-methyl-1H-indazol-3-yl)piperidine-2,6-dione](0.035 g) as an off white solid.

¹H NMR (400 MHz, DMSO-d6): 10.81 (s, 1H), 8.74 (s, 1H), 8.17 (s, 1H), 8.01 (s, 1H), 7.84 (d, J=Hz, 1H), 7.43 (d, J=8.00 Hz, 1H), 7.30 (d, J=8.00 Hz, 1H), 6.68 (d, J=8.00 Hz, 1H), 6.38 (s, 1H), 6.17 (s, 1H), 5.54 (d, J=12.00 Hz, 1H), 4.41-4.46 (m, 2H), 4.18-4.14 (m, 1H), 3.78 (s, 3H), 3.57-3.72 (m, 8H), 3.22-3.33 (m, 1H), 2.58 (d, J=8.00 Hz, 2H), 2.24-0.00 (m, 1H), 2.14-2.15 (m, 1H), 1.60-1.88 (m, 6H), 1.33-1.55 (m, 2H), 0.89 (d, J=8.00 Hz, 3H), 0.71-0.73 (m, 1H), 0.51-0.54 (m, 2H), 0.36-0.37 (m, 1H).

LC-MS (ESI): m/z=827.51 [M+H]⁺

### Example 128: 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-7-fluoro-1-methyl-1H-indazol-3-yl)piperidine-2,6-dione (Compound 471a)

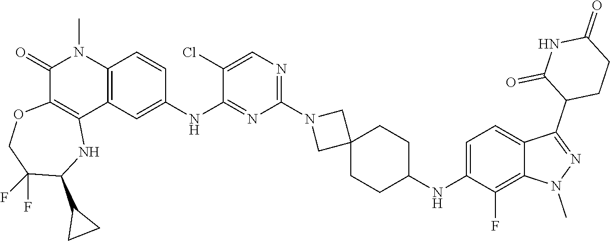 [SMILES: Cn1nc(C2CCC(=O)NC2=O)c2ccc(NC3CCC4(CC3)CN(c3ncc(Cl)c(Nc5ccc6c(c5)c5c(c(=O)n6C)OCC(F)(F)[C@H](C6CC6)N5)n3)C4)c(F)c21]

### Preparation of(E)-1-(4-bromo-2,3-difluoro-phenyl)-N-methoxy-methanimine

To a solution of O-methylhydroxylamine (6.24 g, 74.66 mmol, HCl) in DME (200 mL) was added K₂CO₃ (28.14 g, 203.62 mmol) and 4-bromo-2,3-difluoro-benzaldehyde (15.0 g, 67.87 mmol).

The mixture was stirred at 40° C. for 3 hours. The reaction mixture was filtered to give (E)-1-(4-bromo-2,3-difluoro-phenyl)-N-methoxy-methanimine (50.0 g, 199.97 mmol) as a white solid.

¹H NMR (400 MHz, DMSO-d₆): δ=8.25 (s, 1H), 7.62-7.51 (m, 1H), 7.49-7.38 (m, 1H), 3.93 (s, 3H).

### Preparation of 6-bromo-7-fluoro-1H-indazole

To a solution of (E)-1-(4-bromo-2,3-difluoro-phenyl)-N-methoxy-methanimine (25.0 g, 99.98 mmol) in DME (300 mL) was added N₂H₄·H₂O (30.64 g, 599.90 mmol, 29.69 mL). The reaction was degassed and purged with N₂ 3 times. The mixture was stirred at 100° C. for 16 hours under N₂ atmosphere. The reaction mixture was cooled to 20° C., concentrated under reduced pressure to remove DME, and added into H₂O (500 mL). The precipitate was filtered off to afford 6-bromo-7-fluoro-1H-indazole (42.0 g) as a white solid (two batches).

¹H NMR (400 MHz, DMSO-d₆): δ=13.01 (s, 1H), 8.29-8.19 (m, 1H), 7.55 (d, J=8.4 Hz, 1H), 7.32-7.24 (m, 1H).

### Preparation of 6-bromo-7-fluoro-3-iodo-1H-indazole

To a solution of 6-bromo-7-fluoro-1H-indazole (21.0 g, 97.66 mmol) in DMF (200 mL) was added KOH (10.96 g, 195.33 mmol). The mixture was degassed and purged with N₂ 3 times. 12 (39.66 g, 156.26 mmol) was added at 0° C. and then the mixture was stirred at 25° C. for 3.5 hours under N₂ atmosphere. The reaction mixture was quenched by addition of saturated Na₂SO₃ solution (1 L) at 20° C. The reaction was diluted with H₂O (1 L) and filtered to give 6-bromo-7-fluoro-3-iodo-1H-indazole (60.0 g) as a yellow solid (two batches).

LC-MS (ESI): m/z=340.7 [M+H]⁺.

### Preparation of 6-bromo-7-fluoro-3-iodo-1-methyl-indazole

A mixture of 6-bromo-7-fluoro-3-iodo-1H-indazole (28.0 g, 82.13 mmol), Mel (23.32 g, 164.26 mmol, 10.23 mL) in DMF (300 mL) was degassed and purged with N₂ 3 times. K₂CO₃ (34.05 g, 246.39 mmol) was added, and the mixture was stirred at 40° C. for 3.5 hours under N₂ atmosphere.

The reaction mixture was quenched by addition of saturated aqueous NH₄Cl (200 mL) at 0° C., and then diluted with H₂O (200 mL). The reaction was extracted with EtOAc (200 mL×3). The combined organic layers were washed with H₂O (200 mL×3), dried over Na₂SO₄, filtered, and concentrated under reduced pressure to give a residue. The residue was purified by column chromatography (SiO₂, Petroleum ether/Ethyl acetate=200/1 to 100/1) to afford 6-bromo-7-fluoro-3-iodo-1-methyl-indazole (40.0 g) as a light-yellow solid (two batches).

LC-MS (ESI): m/z=356.7 [M+H]⁺.

### Preparation of 3-(2,6-bis(benzyloxy)pyridin-3-yl)-6-bromo-7-fluoro-1-methyl-1H-indazole

To a solution of 6-bromo-7-fluoro-3-iodo-1-methyl-indazole (2.0 g, 5.67 mmol), (2,6-bis(benzyloxy)pyridin-3-yl) boronic acid (1.9 g, 5.67 mmol) and K₃PO₄ (3.6 g, 17.01 mmol) in 1,4-dioxane (10 ml), was added Pd(PPh₃)₄(0.65 g, 0.56 mmol) under nitrogen. The reaction mixture was stirred at 100° C. for 12 h. The reaction mixture was filtered through a celite bed, and the filtrate was concentrated under reduced pressure to get a residue which was purified by flash column chromatography (SiO₂, 100-200 mesh, 60% ethyl acetate in petroleum ether) to afford 3-(2,6-bis(benzyloxy)pyridin-3-yl)-6-bromo-7-fluoro-1-methyl-1H-indazole (1.2 g) as an off white solid.

LC-MS (ESI): m/z=520.18 [M+H]⁺.

### Preparation of tert-butyl 7-((3-(2,6-bis(benzyloxy)pyridin-3-yl)-7-fluoro-1-methyl-1H-indazol-6-yl) amino)-2-azaspiro [3.5]nonane-2-carboxylate

To a solution of 3-(2,6-bis(benzyloxy)pyridin-3-yl)-6-bromo-7-fluoro-1-methyl-1H-indazole (0.8 g, 1.54 mmol) and tert-butyl 7-amino-2-azaspiro[3.5]nonane-2-carboxylate (0.37 g, 1.54 mmol) in 1,4-dioxane (1.6 mL) under nitrogen was added BrettPhos Pd-G3 (0.14 g, 0.15 mmol) and NaOtBu (2M in THF) (2.3 mL, 4.62 mmol). The resulting mixture was stirred at 100° C. for 10 h.

After completion, the reaction mixture was filtered through a celite bed (15.5 g), and the filtrate was concentrated under reduced pressure and purified by flash column chromatography (SiO₂, 100-200 mesh, 60% ethyl acetate in petroleum ether) to afford tert-butyl 7-((3-(2,6-bis(benzyloxy)pyridin-3-yl)-7-fluoro-1-methyl-1H-indazol-6-yl) amino)-2-azaspiro [3.5]nonane-2-carboxylate (0.35 g) as a brown solid.

LC-MS (ESI): m/z=678.68 [M+H]⁺.

### Preparation of tert-butyl 7-((3-(2,6-dioxopiperidin-3-yl)-7-fluoro-1-methyl-1H-indazol-6-yl) amino)-2-azaspiro [3.5]nonane-2-carboxylate

To a suspension of tert-butyl 7-((3-(2,6-bis(benzyloxy)pyridin-3-yl)-7-fluoro-1-methyl-1H-indazol-6-yl) amino)-2-azaspiro [3.5]nonane-2-carboxylate (0.35 g, 0.51 mmol) in THF (10 mL), was added 20% Pd(OH)₂ on carbon (100% w/w). The reaction mixture was hydrogenated using a par shaker (100 psi) at room temperature for 16 h. After completion, the reaction mixture was filtered through a celite bed and the filtrate was concentrated under reduced pressure to afford tert-butyl 7-((3-(2,6-dioxopiperidin-3-yl)-7-fluoro-1-methyl-1H-indazol-6-yl) amino)-2-azaspiro [3.5]nonane-2-carboxylate (0.1 g) as an off-white solid which was used in the next step without further purification.

LC-MS (ESI): m/z=500.06 [M+H]⁺

### Preparation of 3-(6-((2-azaspiro[3.5]nonan-7-yl)amino)-7-fluoro-1-methyl-1H-indazol-3-yl)piperidine-2,6-dione

To a suspension of tert-butyl 7-((3-(2,6-dioxopiperidin-3-yl)-7-fluoro-1-methyl-1H-indazol-6-yl) amino)-2-azaspiro [3.5]nonane-2-carboxylate (0.1 g, 0.20 mmol) in DCM (2.5 mL) was added TFA (0.5 mL) at 0° C. The reaction mixture was stirred at room temperature for 3 h. After completion, the reaction mixture was concentrated under reduced pressure and purified by trituration with pentane (10 mL) to afford 3-(6-((2-azaspiro[3.5]nonan-7-yl)amino)-7-fluoro-1-methyl-1H-indazol-3-yl)piperidine-2,6-dione (0.1 g) as a brown gum.

LC-MS (ESI): m/z=400.25 [M+H]⁺

### Preparation of 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-7-fluoro-1-methyl-1H-indazol-3-yl)piperidine-2,6-dione

To a solution of 3-(6-((2-azaspiro[3.5]nonan-7-yl)amino)-7-fluoro-1-methyl-1H-indazol-3-yl)piperidine-2,6-dione (0.1 g, 0.25 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.12 mmol) in DMSO (2.0 mL) was added N,N-diisopropylethylamine (0.5 ml) at room temperature. The resulting mixture was stirred at 110° C. for 4 h. After completion of the reaction, the reaction mixture was concentrated under reduced pressure and purified by prep-HPLC to afford 3-(6-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)amino)-7-fluoro-1-methyl-1H-indazol-3-yl)piperidine-2,6-dione (0.027 g) as a pale yellow solid.

LC-MS (ESI): m/z=831.46 [M+H]⁺

¹H NMR: 400 MHz, DMSO-d₆: 10.85 (s, 1H), 8.75 (s, 1H), 8.20 (s, 1H), 8.02 (s, 1H), 7.84 (dd, J=2.00, 9.00 Hz, 1H), 7.44 (d, J=9.20 Hz, 1H), 7.26 (d, J=8.80 Hz, 1H), 6.76 (t, J=7.60 Hz, 1H), 6.14 (s, 1H), 5.05 (br s, 1H), 4.43-4.42 (m, 2H), 4.24-4.22 (m, 1H), 4.00 (s, 3H), 3.64-3.50 (m, 7H), 3.27-3.24 (m, 2H), 2.60-2.52 (m, 2H), 2.33-2.29 (m, 1H), 2.16-2.11 (m, 1H), 1.83-1.74 (m, 4H), 1.56-1.51 (m, 2H), 1.33-1.29 (m, 3H), 0.80-0.67 (m, 1H), 0.53-0.49 (m, 2H), 0.40-0.35 (m, 1H).

### Example 129: 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (Compound 460b)

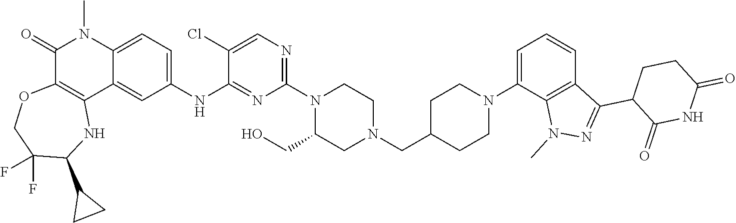 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)[C@@H](CO)C4)CC3)c21]

### Preparation of (1-(3-(2,6-bis(benzyloxy)pyridin-3-yl)-1-methyl-1H-indazol-7-yl)piperidin-4-yl)methanol

To a stirred solution of 3-(2,6-bis(benzyloxy)pyridin-3-yl)-7-bromo-1-methyl-1H-indazole (2.0 g, 3.9 mmol) and piperidin-4-ylmethanol (1.38 g, 11.9 mmol) in 1,4-dioxane (40 mL) was added cesium carbonate (3.9 g, 11.9 mmol). Pd-PEPPSI-IHeptCl (0.06 g, 0.2 mmol) was added under argon, and the reaction mixture was stirred at 100° C. for 16 h. The reaction mixture was cooled to room temperature and filtered through a celite bed, washed with ethyl acetate (100 mL), and the filtrate was concentrated to get a residue which was purified by flash column chromatography (SiO₂, 60-120 mesh, 25% ethyl acetate in petroleum ether) to afford (1-(3-(2,6-bis(benzyloxy)pyridin-3-yl)-1-methyl-1H-indazol-7-yl)piperidin-4-yl)methanol (0.45 g) as a brown semi-solid.

LCMS (ESI): m/z=534.64 [M+H]⁺.

### Preparation of 3-(7-(4-(hydroxymethyl)piperidin-1-yl)-1-methyl-1H-indazol-3-yl)piperidine-2,6-dione

To a stirred solution of (1-(3-(2,6-bis(benzyloxy)pyridin-3-yl)-1-methyl-1H-indazol-7-yl)piperidin-4-yl)methanol (0.45 g, 0.84 mmol) in THF (18 mL) was added 20% Pd(OH)₂ on carbon (0.45 g) at room temperature. The reaction was stirred at room temperature under H₂ (80 psi) atmosphere for 16 h. The reaction mixture was filtered through a celite bed, washed with DCM, and concentrated under reduced pressure to afford 3-(7-(4-(hydroxymethyl)piperidin-1-yl)-1-methyl-1H-indazol-3-yl)piperidine-2,6-dione (0.35 g) as an off-white solid which was used in the next step without further purification.

LCMS (ESI): m/z=357.44 [M+H]⁺.

### Preparation of 1-(3-(2,6-dioxopiperidin-3-yl)-1-methyl-1H-indazol-7-yl)piperidine-4-carbaldehyde

To a solution of 3-(7-(4-(hydroxymethyl)piperidin-1-yl)-1-methyl-1H-indazol-3-yl)piperidine-2,6-dione (0.35 g, 0.98 mmol) in DCM (17.5 mL) was added Dess-Martin periodinane (1.04 g, 2.45 mmol) at 0° C. The reaction mixture was stirred at room temperature for 3 h. The reaction mixture was filtered and washed with DCM (20 mL). The filtrate was washed with a saturated solution of sodium bicarbonate (20 mL). The collected organic layer was dried over sodium sulfate and concentrated under reduced pressure to afford 1-(3-(2,6-dioxopiperidin-3-yl)-1-methyl-1H-indazol-7-yl)piperidine-4-carbaldehyde (0.30 g) as a brown semi-solid which was used in the next step without further purification.

LC-MS (ESI): m/z=355.38 [M+H]⁺.

### Preparation of tert-butyl (R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazine-1-carboxylate

To a solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.21 mmol) and tert-butyl (R)-3-(hydroxymethyl)piperazine-1-carboxylate (0.27 g, 1.26 mmol) in DMSO (2 mL) was added N,N-diisopropylethylamine (0.1 mL, 0.63 mmol) and reaction mixture was stirred at 100° C. for 16 h. The reaction mixture was poured into ice-cold water and stirred for 10 min. The precipitate obtained was filtered and dried to get crude tert-butyl (R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazine-1-carboxylate (0.13 g) as an off white solid that was used in the next step without purification. LC-MS (ESI): m/z=648.57 [M+H]⁺

### Preparation of (S)-10-((5-chloro-2-((R)-2-(hydroxymethyl)piperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one

To a solution of tert-butyl (R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazine-1-carboxylate (0.13 g, 0.20 mmol)) in DCM (2.6 mL) was added TFA (0.65 mL) at 0° C. The reaction mixture was stirred at room temperature for 2 h. The reaction mixture was concentrated under reduced pressure, co-distilled with diethyl ether, and washed with n-pentane to afford (S)-10-((5-chloro-2-((R)-2-(hydroxymethyl)piperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one (0.12 g) as a brown semi-solid.

LC-MS (ESI): m/z=546.16 [M+H]⁺

### Preparation of 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione

To a solution of tert-butyl (S)-10-((5-chloro-2-((R)-2-(hydroxymethyl)piperazin-1-yl)pyrimidin-4-yl)amino)-2-cyclopropyl-3,3-difluoro-7-methyl-1,2,3,4-tetrahydro-[1,4]oxazepino[2,3-c]quinolin-6(7H)-one (0.10 g, 0.18 mmol), 1-(3-(2,6-dioxopiperidin-3-yl)-1-methyl-1H-indazol-7-yl)piperidine-4-carbaldehyde (0.09 g, 0.12 mmol), and sodium acetate anhydrous (0.044 g, 0.54 mmol) in DCM (5.0 mL) was added acetic acid (0.10 mL). The reaction was stirred for 3 h.

Sodium triacetoxy borohydride (0.11 g, 0.54 mmol) was then added at 0° C. under a nitrogen atmosphere, and the resulting mixture was stirred at room temperature for 16 h. The reaction mixture was quenched with water (50 mL) and extracted with ethyl acetate (2×100 mL). The combined organic layers were washed with brine, dried over anhydrous Na₂SO₄, filtered, and concentrated under reduced pressure to provide a residue which was purified by prep-HPLC to afford 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-(hydroxymethyl)piperazin-1-yl)methyl)piperidin-1-yl)-1-methyl-1H-indazol-3-yl)piperidine-2,6-dione (0.032 g) as an off white solid.

LC-MS (ESI): m/z=886.42 [M+H]⁺.

¹H NMR (400 MHz, DMSO-d6): δ 11.87 (s, 1H), 8.80 (s, 1H), 8.46 (s, 1H), 8.22 (s, 1H), 8.04 (s, 1H), 7.70 (d, J=7.60 Hz, 1H), 7.43-7.35 (m, 2H), 7.03-7.00 (m, 2H), 6.26 (br s, 1H), 4.51-4.31 (m, 9H), 3.71-3.70 (m, 1H), 3.56 (s, 3H), 3.20-3.18 (br, 3H), 3.04-3.01 (m, 2H), 2.81-2.79 (m, 1H), 2.61-2.58 (m, 5H), 2.31-2.30 (m, 1H), 2.18-2.14 (m, 3H), 1.88-1.85 (m, 4H), 1.36-1.33 (m, 3H), 0.71-0.70 (m, 1H), 0.53-0.52 (m, 2H), 0.36-0.35 (m, 1H).

### Example 130: 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2-methylpiperazin-1-yl)-1-methyl-1H-indazol-3-yl)piperidine-2,6-dione (Compound 475a)

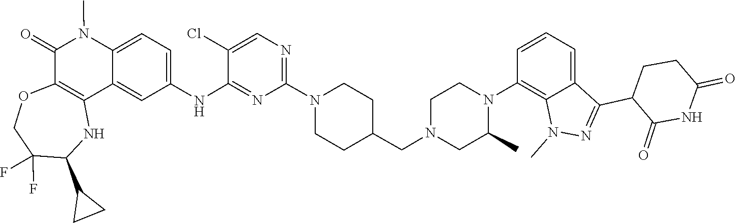 [SMILES: C[C@H]1CN(CC2CCN(c3ncc(Cl)c(Nc4ccc5c(c4)c4c(c(=O)n5C)OCC(F)(F)[C@H](C5CC5)N4)n3)CC2)CCN1c1cccc2c(C3CCC(=O)NC3=O)nn(C)c12]

### Preparation of tert-butyl (S)-4-(3-(2,6-bis(benzyloxy)pyridin-3-yl)-1-methyl-1H-indazol-7-yl)-3-methylpiperazine-1-carboxylate

To a stirred solution of 3-(2,6-bis(benzyloxy)pyridin-3-yl)-7-bromo-1-methyl-1H-indazole (3.00 g, 5.99 mmol), tert-butyl (S)-3-methylpiperazine-1-carboxylate (3.60 g, 17.99 mmol), and cesium carbonate (5.86 g, 17.99 mmol) in dioxane (30 mL) was added Pd-PEPPSI-iHeptCl (0.29 g, 0.30 mmol) under argon. The reaction mixture was heated to 100° C. for 18 h. The reaction mixture was filtered through a pad of celite and washed with excess of EtOAc (500 mL). The collected filtrate was concentrated under vacuum and purified by flash chromatography (230-400 mesh silica gel and 10%-15% of ethyl acetate in petroleum ether as eluent) to afford tert-butyl (S)-4-(3-(2,6-bis(benzyloxy)pyridin-3-yl)-1-methyl-1H-indazol-7-yl)-3-methylpiperazine-1-carboxylate (1.80 g) as a pale yellow semi-solid.

LC-MS (ESI): m/z=620.55 [M+H]⁺.

### Preparation of (S)-6-(benzyloxy)-5-(1-methyl-7-(2-methylpiperazin-1-yl)-1H-indazol-3-yl) pyridin-2-ol

To a solution of tert-butyl (S)-4-(3-(2,6-bis(benzyloxy)pyridin-3-yl)-1-methyl-1H-indazol-7-yl)-3-methylpiperazine-1-carboxylate (1.80 g, 2.90 mmol) in DCM (18 mL) was added TFA (14.40 mL) at 0° C. The reaction mixture was stirred at room temperature for 2 h. The reaction mixture was concentrated under reduced pressure to afford (S)-6-(benzyloxy)-5-(1-methyl-7-(2-methylpiperazin-1-yl)-1H-indazol-3-yl) pyridin-2-ol (1.82 g) as a pale brown semi-solid.

LC-MS (ESI): m/z=430.29 [M+H]⁺

### Preparation of benzyl (S)-4-((4-(3-(2-(benzyloxy)-6-hydroxypyridin-3-yl)-1-methyl-1H-indazol-7-yl)-3-methylpiperazin-1-yl)methyl)piperidine-1-carboxylate

To a solution of (S)-6-(benzyloxy)-5-(1-methyl-7-(2-methylpiperazin-1-yl)-1H-indazol-3-yl) pyridin-2-ol (1.82 g, 4.24 mmol), benzyl 4-formylpiperidine-1-carboxylate (1.26 g, 5.08 mmol), acetic acid (1.82 mL), and sodium acetate (1.04 g, 12.71 mmol) in THF (72 mL) was added sodium triacetoxy borohydride (1.80 g, 8.47 mmol) portion wise at 0° C. The reaction mixture was allowed to stirred at room temperature for 16 h. The reaction mixture was quenched with water (200 mL) and extracted with ethyl acetate (500 mL). The combined organic layers were washed with brine (50 mL), dried over anhydrous Na₂SO₄, filtered, and concentrated under vacuum to provide a residue which was purified by flash chromatography (80-90% of ethyl acetate in petroleum ether as eluent) to afford benzyl (S)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.68 g) as a pale yellow semi-solid.

LC-MS (ESI): m/z=661.41 [M+H]⁺

### Preparation of 3-(1-methyl-7-((S)-2-methyl-4-(piperidin-4-ylmethyl) piperazin-1-yl)-1H-indazol-3-yl)piperidine-2,6-dione

To a solution of benzyl (S)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.65 g, 0.98 mmol) in THF (52 mL) was added 20% Pd (OH)₂ on carbon (1.25 g, 7.87 mmol). The reaction mixture was stirred under hydrogen atmosphere (80 psi) at room temperature for 18 h. The reaction mixture was diluted with DCM (100 mL) and filtered through a celite bed and washed with 30% THF in DCM (700 mL). The collected filtrate was concentrated under vacuum to afford 3-(1-methyl-7-((S)-2-methyl-4-(piperidin-4-ylmethyl) piperazin-1-yl)-1H-indazol-3-yl)piperidine-2,6-dione (0.42 g) as a pale brown semi-solid which was used in the next step without purification.

LC-MS (ESI): m/z=439.32 [M+H]⁺

### Preparation of 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2-methylpiperazin-1-yl)-1-methyl-1H-indazol-3-yl)piperidine-2,6-dione

To a stirred solution of 3-(1-methyl-7-((S)-2-methyl-4-(piperidin-4-ylmethyl) piperazin-1-yl)-1H-indazol-3-yl)piperidine-2,6-dione (0.21 g, 0.48 mmol) and (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.09 g, 0.19 mmol) in DMSO (2.1 mL) was added DIPEA (2.1 mL). The reaction mixture was heated to 100° C. for 18 h. The reaction mixture was quenched with ice cold water (100 mL), and the precipitated solid was filtered and dried under vacuum to obtain a crude product which was purified by prep-HPLC to afford 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2-methylpiperazin-1-yl)-1-methyl-1H-indazol-3-yl)piperidine-2,6-dione (0.045 g) as an off white solid.

LC-MS (ESI): m/z=870.50 [M+H]⁺

¹H NMR: (400 MHz, DMSO-d6): δ 10.91 (s, 1H), 9.35 (s, 1H), 8.89 (s, 1H), 8.13 (s, 1H), 7.74 (d, J=7.60 Hz, 1H), 7.57 (dd, J=2.40, 8.00 Hz, 1H), 7.43 (d, J=9.20 Hz, 1H), 7.21-7.23 (m, 1H), 7.08-7.12 (m, 1H), 6.25 (s, 1H), 4.32-4.35 (m, 5H), 4.27 (s, 3H), 3.57 (s, 4H), 2.99-3.02 (m, 8H), 2.82-2.86 (m, 2H), 2.36 (s, 1H), 2.15-2.18 (m, 3H), 1.75 (d, J=11.60 Hz, 2H), 1.36 (s, 3H), 1.13-1.16 (m, 2H), 0.86 (d, J=6.00 Hz, 3H), 0.70-0.71 (m, 1H), 0.51-0.53 (m, 2H), 0.34 (d, J=4.40 Hz, 1H).

### Example 131: 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperazin-1-yl)methyl)piperidin-1-yl)-1-methyl-1H-indazol-3-yl)piperidine-2,6-dione (Compound 476a)

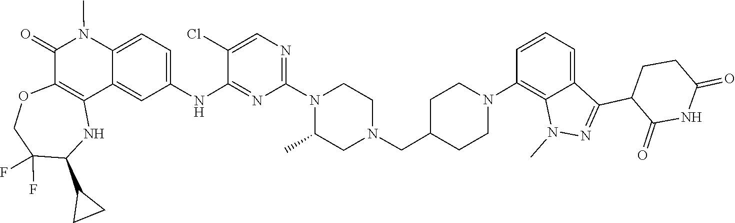 [SMILES: C[C@H]1CN(CC2CCN(c3cccc4c(C5CCC(=O)NC5=O)nn(C)c34)CC2)CCN1c1ncc(Cl)c(Nc2ccc3c(c2)c2c(c(=O)n3C)OCC(F)(F)[C@H](C3CC3)N2)n1]

### Preparation of tert-butyl (S)-4-((1-((benzyloxy)carbonyl) piperidin-4-yl) methyl)-2-methylpiperazine-1-carboxylate

A solution of tert-butyl (S)-2-methylpiperazine-1-carboxylate (4.0 g, 19.97 mmol) in THF (80.0 mL) was added benzyl 4-formylpiperidine-1-carboxylate (4.94 g, 19.97 mmol) and acetic acid (0.40 mL). The reaction mixture was stirred under nitrogen atmosphere for 2 h, followed by addition of sodium triacetoxyborohydride (12.70 g, 59.92 mmol). The mixture was stirred at room temperature for 18 h. The reaction mixture was diluted with DCM (300 mL), quenched with sat.

NH₄Cl solution (300 mL), dried over anhydrous Na₂SO₄, filtered, and concentrated under vacuum to obtain a residue which was purified by flash chromatography using (230-400 mesh silica gel, 14% of EtOAc in petroleum ether as eluent) to afford tert-butyl (S)-4-((1-((benzyloxy)carbonyl) piperidin-4-yl) methyl)-2-methylpiperazine-1-carboxylate (5.69 g) as a pale brown semi-solid.

LCMS (ESI): m/z=432.61 [M+H]⁺

### Preparation of tert-butyl (S)-2-methyl-4-(piperidin-4-ylmethyl) piperazine-1-carboxylate

To a solution of tert-butyl (S)-4-((1-((benzyloxy)carbonyl) piperidin-4-yl) methyl)-2-methylpiperazine-1-carboxylate (6.80 g, 15.76 mmol) in THF (210.0 mL) was added 10% Pd/C (3.40 g, 50% w/w). The reaction mixture was stirred and hydrogen atmosphere (Parr, 80 psi) at room temperature for 18 h. The reaction mixture was diluted with DCM (300 mL), filtered through a pad of celite, concentrated under vacuum, and triturated with n-pentane (50 mL) to afford tert-butyl (S)-2-methyl-4-(piperidin-4-ylmethyl) piperazine-1-carboxylate (4.21 g) as a pale brown semi-solid.

### Preparation of tert-butyl (S)-4-((1-(3-(2,6-bis(benzyloxy)pyridin-3-yl)-1-methyl-1H-indazol-7-yl) piperidin-4-yl) methyl)-2-methylpiperazine-1-carboxylate

To a stirred solution of tert-butyl (S)-2-methyl-4-(piperidin-4-ylmethyl) piperazine-1-carboxylate (0.50 g, 1.68 mmol) in dioxane (5.0 mL), was added 3-(2,6-bis(benzyloxy)pyridin-3-yl)-7-bromo-1-methyl-1H-indazole (0.42 g, 0.84 mmol) and Cs₂CO₃ (1.64 g, 5.04 mmol). To the reaction mixture was added Pd-PEPPSI-iHeptCl (0.16 g, 0.01 mmol) under argon, and the reaction mixture was heated to 110° C. for 12 h. The reaction mixture was diluted with EtOAc (100 mL), quenched with water (30 mL), dried over anhydrous Na₂SO₄, filtered, and concentrated under vacuum to obtain crude product that was then purified by using (230-400 mesh silica gel, 22% of EtOAc in petroleum ether as eluent) to afford tert-butyl (S)-4-((1-(3-(2,6-bis(benzyloxy)pyridin-3-yl)-1-methyl-1H-indazol-7-yl) piperidin-4-yl) methyl)-2-methylpiperazine-1-carboxylate (0.62 g) as a pale brown semi-solid.

LCMS (ESI): m/z=717.71 [M+H]⁺

### Preparation of tert-butyl (2S)-4-((1-(3-(2,6-dioxopiperidin-3-yl)-1-methyl-1H-indazol-7-yl) piperidin-4-yl) methyl)-2-methylpiperazine-1-carboxylate

To a solution of tert-butyl (S)-4-((1-(3-(2,6-bis(benzyloxy)pyridin-3-yl)-1-methyl-1H-indazol-7-yl) piperidin-4-yl) methyl)-2-methylpiperazine-1-carboxylate (3.62 g, 5.05 mmol) in tetrahydrofuran (160.0 mL) was added Pd(OH)₂ on carbon, 20% Pd (1.31 g, 50% w/w). The reaction mixture was stirred under hydrogen atmosphere (80 psi) at room temperature for 18 h.

The reaction mixture was diluted with 30% of THF in DCM (600 mL), concentrated under vacuum, and triturated with n-pentane (30 mL) to afford tert-butyl (2S)-4-((1-(3-(2,6-dioxopiperidin-3-yl)-1-methyl-1H-indazol-7-yl) piperidin-4-yl) methyl)-2-methylpiperazine-1-carboxylate (2.42 g) as a pale brown semi-solid.

LCMS (ESI): m/z=539.69 [M+H]⁺.

### Preparation of 3-(1-methyl-7-(4-(((S)-3-methylpiperazin-1-yl) methyl) piperidin-1-yl)-1H-indazol-3-yl) piperidine-2,6-dione

To a stirred solution of tert-butyl (2S)-4-((1-(3-(2,6-dioxopiperidin-3-yl)-1-methyl-1H-indazol-7-yl) piperidin-4-yl) methyl)-2-methylpiperazine-1-carboxylate (0.5 g, 0.93 mmol) in DCM (1.5 mL) was added TFA (0.5 mL) at 0° C. The reaction mixture was stirred at room temperature for 2 h. The reaction mixture was concentrated under vacuum and triturated with n-pentane (10 mL) to afford 3-(1-methyl-7-(4-(((S)-3-methylpiperazin-1-yl) methyl) piperidin-1-yl)-1H-indazol-3-yl) piperidine-2,6-dione (0.50 g, TFA salt) as a pale brown semi-solid.

LCMS (ESI): m/z=439.57 [M+H]⁺.

### Preparation of 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperazin-1-yl)methyl)piperidin-1-yl)-1-methyl-1H-indazol-3-yl)piperidine-2,6-dione

To a stirred solution of 3-(1-methyl-7-(4-(((S)-3-methylpiperazin-1-yl) methyl) piperidin-1-yl)-1H-indazol-3-yl) piperidine-2,6-dione (0.3 g, 0.68 mmol), (S)-2-cyclopropyl-10-((2,5-dichloropyrimidin-4-yl) amino)-3,3-difluoro-7-methyl-1,2,3,4-tetrahydro-[1,4]oxazepino[2,3-c]quinolin-6(7H)-one (0.09 g, 0.20 mmol) in DMSO (6.0 mL) was added DIPEA (3.0 mL). The reaction mixture was heated to 135° C. for 18 h. The reaction mixture was poured into ice cold water (20 mL), and the precipitated solid was filtered and dried under vacuum to afford crude product which was purified by prep HPLC to afford 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperazin-1-yl)methyl)piperidin-1-yl)-1-methyl-1H-indazol-3-yl)piperidine-2,6-dione (0.04 g) as an off white solid.

LCMS (ESI): m/z=870.53 [M+H]⁺

¹H NMR (400 MHz, DMSO-d6): δ 10.87 (s, 1H), 8.80 (s, 1H), 8.21 (d, J=1.60 Hz, 1H), 8.05 (s, 1H), 7.71 (d, J=10.00 Hz, 1H), 7.43 (d, J=9.20 Hz, 1H), 7.36 (dd, J=2.80, 6.20 Hz, 1H), 7.01 (dd, J=2.80, 8.40 Hz, 2H), 6.23 (s, 1H), 4.42 (br s, 1H), 4.32-4.31 (m, 3H), 4.24 (s, 3H), 3.56 (s, 3H), 3.23 (br s, 3H), 3.04-3.01 (m, 1H), 2.81-2.78 (m, 1H), 2.67-2.67 (m, 3H), 2.62-2.61 (m, 5H), 2.31-2.18 (m, 5H), 2.05-2.03 (m, 1H), 1.86-1.74 (m, 3H), 1.16 (d, J=6.40 Hz, 3H), 0.70 (d, J=4.80 Hz, 2H), 0.51 (d, J=5.20 Hz, 2H), 0.35-0.34 (m, 1H).

### Example 132: 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperidin-4-yl)-1-methyl-1H-indazol-3-yl)piperidine-2,6-dione (Compound 479a)

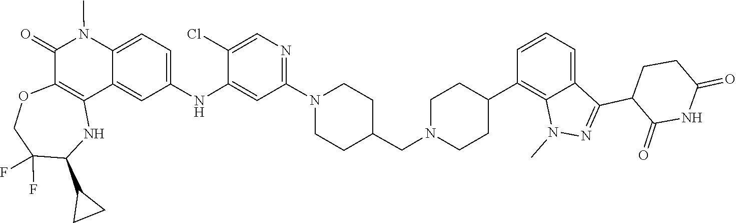 [SMILES: Cn1nc(C2CCC(=O)NC2=O)c2cccc(C3CCN(CC4CCN(c5cc(Nc6ccc7c(c6)c6c(c(=O)n7C)OCC(F)(F)[C@H](C7CC7)N6)c(Cl)cn5)CC4)CC3)c21]

### Preparation of (1-(5-chloro-4-iodopyridin-2-yl)piperidin-4-yl)methanol

To a solution of piperidin-4-ylmethanol (0.98 g, 8.54 mmol) and DIPEA (4.2 mL, 23.30 mmol) in DMSO (20 mL) was added 5-chloro-2-fluoro-4-iodopyridine (2.0 g, 7.76 mmol). The reaction mixture was stirred at room temperature for 3 h. The reaction mixture was concentrated under reduced pressure, diluted with water (20 mL), and extracted with ethyl acetate (3×100 mL). The combined organic layer was concentrated under reduced pressure to provide a crude product that was purified by flash column chromatography (SiO₂, 60-120, 25% ethyl acetate in petroleum ether) to afford (1-(5-chloro-4-iodopyridin-2-yl)piperidin-4-yl)methanol (3.1 g).

LCMS (ESI): m/z=353.26 [M+H]⁺.

### Preparation of (S)-10-((5-chloro-2-(4-(hydroxymethyl) piperidin-1-yl)pyridin-4-yl)amino)-2-cyclopropyl-3,3-difluoro-7-methyl-1,2,3,4-tetrahydro-[1,4]-oxazepino[2,3-c]quinolin-6(7H)-one

To solution of (1-(5-chloro-4-iodopyridin-2-yl)piperidin-4-yl)methanol (2 g, 5.67 mmol) and (S)-10-amino-2-cyclopropyl-3,3-difluoro-7-methyl-1,2,3,4-tetrahydro-[1,4]oxazepino[2,3-c]quinolin-6(7H)-one (1.82 g, 5.67 mmol) and cesium carbonate (5.54 g, 17.01 mmol) in 1,4-dioxane (20 mL) was added Pd-PEPPSI-iHeptCl (0.27 g, 0.56 mmol) under N₂. The reaction mixture was stirred at 100° C. for 16 h. The reaction mixture was quenched with ice cold water (50 mL) and extracted with ethyl acetate (2×200). The combined organic layer was concentrated under reduced pressure to provide a crude product which was purified by flash column chromatography (SiO₂, 60-120, 50-60% ethyl acetate in petroleum ether) to afford (S)-10-((5-chloro-2-(4-(hydroxymethyl) piperidin-1-yl)pyridin-4-yl)amino)-2-cyclopropyl-3,3-difluoro-7-methyl-1,2,3,4-tetrahydro-[1,4]-oxazepino[2,3-c]quinolin-6(7H)-one as solid (1.1 g).

LC-MS (ESI): m/z=546.55 [M+H]⁺

### Preparation of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde

To a solution of (S)-10-((5-chloro-2-(4-(hydroxymethyl) piperidin-1-yl)pyridin-4-yl)amino)-2-cyclopropyl-3,3-difluoro-7-methyl-1,2,3,4-tetrahydro-[1,4]-oxazepino[2,3-c]quinolin-6(7H)-one (1.0 g, 1.83 mmol,) in DCM (10 mL) was added Dess-Martin periodinane (1.55 g, 3.66 mmol) at 0° C. The reaction mixture stirred at room temperature for 4 h. The reaction mixture was quenched with ice cold water (20 mL) and extracted with ethyl acetate (2×100 mL). The combined organic layer was concentrated under reduced pressure to provide a crude compound that was purified by trituration to afford (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde as a solid (0.52 g). LC-MS (ESI): m/z=544.50 [M+H]⁺

### Preparation of 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperidin-4-yl)-1-methyl-1H-indazol-3-yl)piperidine-2,6-dione

To a solution of 3-(1-methyl-7-(piperidin-4-yl)-1H-indazol-3-yl)piperidine-2,6-dione (0.30 g, 0.92 mmol), sodium acetate (0.22 g, 2.76 mmol), and AcOH (0.1 mL) in THF (5 mL) was added (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde (0.50 g, 0.92 mmol). The reaction mixture was stirred at room temperature for 2 h. After that, sodium triacetoxyborohydride (0.58 g, 2.76 mmol) was added to the reaction mixture which was stirred at room temperature for 14 h. The reaction mixture was quenched with ice cold water (50 mL) and extracted with ethyl acetate (2×100 mL). The combined organic layer was concentrated under reduced pressure to provide a crude product, which was purified by prep-HPLC to afford 3-(7-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperidin-4-yl)-1-methyl-1H-indazol-3-yl)piperidine-2,6-dione (0.045 g).

LC-MS (ESI): m/z=854.53 [M+H]⁺.

¹H NMR (400 MHz, DMSO-d6): 8.07 (br s, 1H), 8.00 (s, 1H), 7.92 (s, 1H), 7.54-7.45 (m, 3H), 7.23 (d, J=8.0 Hz, 1H), 7.10-7.03 (m, 1H), 6.41 (br s, 1H), 6.08 (s, 1H), 4.50-4.32 (m, 3H), 4.19 (s, 3H), 3.98 (t, J=14.8 Hz, 2H). 3.60 (s, 1H), 3.20-3.10 (m, 2H), 2.96 (d, J=10.8 Hz, 2H), 2.70-2.58 (m, 3H), 2.32 (s, 1H), 2.12-2.00 (m, 5H), 1.85 (s, 5H), 1.72-1.60 (m, 6H), 1.35 (br s, 1H), 1.10-1.0 (m, 2H), 0.70-0.60 (m, 1H), 0.56-0.50 (m, 2H), 0.46-0.30 (m, 1H).

### Example 133: 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,2-dimethylpiperazin-1-yl)methyl)piperidin-1-yl)-1-methyl-1H-indazol-3-yl)piperidine-2,6-dione (Compound 480a)

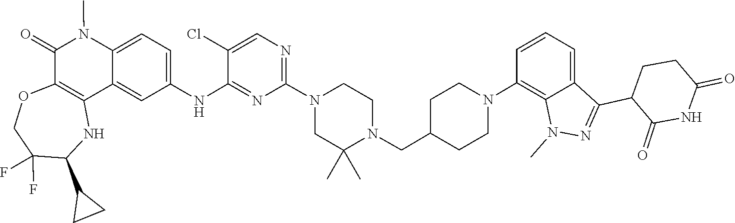 [SMILES: Cn1nc(C2CCC(=O)NC2=O)c2cccc(N3CCC(CN4CCN(c5ncc(Cl)c(Nc6ccc7c(c6)c6c(c(=O)n7C)OCC(F)(F)[C@H](C7CC7)N6)n5)CC4(C)C)CC3)c21]

### Preparation of tert-butyl 4-((1-((benzyloxy)carbonyl)piperidin-4-yl)methyl)-3,3-dimethylpiperazine-1-carboxylate

To a solution of tert-butyl 3,3-dimethylpiperazine-1-carboxylate (2.5 g, 11.67 mmol) and benzyl 4-formylpiperidine-1-carboxylate (3.4 g, 14.00 mmol) in DCM (50 mL) were added acetic acid (0.7 mL, 11.67 mmol) and sodium triacetoxy borohydride (2.35 g, 35.03 mmol) at 0° C. under nitrogen atmosphere. The reaction mixture was stirred at room temperature for 16 h. The reaction mixture was quenched with water (200 mL) and extracted with ethyl acetate (3×100 mL). The combined organic layers were washed with brine, dried over anhydrous Na₂SO₄, filtered, and concentrated under reduced pressure to provide a crude product which was purified by flash column chromatography (SiO₂, 60-120 mesh, 25% ethyl acetate in petroleum ether to afford tert-butyl 4-((1-((benzyloxy)carbonyl)piperidin-4-yl)methyl)-3,3-dimethylpiperazine-1-carboxylate (2.8 g) as a yellow semi-solid.

LCMS (ESI): m/z=446.64 [M+H]⁺.

### Preparation of tert-butyl 3,3-dimethyl-4-(piperidin-4-ylmethyl)piperazine-1-carboxylate

To a solution of tert-butyl 4-((1-((benzyloxy)carbonyl)piperidin-4-yl)methyl)-3,3-dimethylpiperazine-1-carboxylate (2.0 g, 1.57 mmol) in THF (60 mL) was added 20% Pd(OH)₂ on carbon (2.0 g, 100% w/w). The reaction mixture was stirred under hydrogen pressure (80 psi) at room temperature for 16 h. The reaction mixture was diluted with DCM (50 mL) and filtered through a celite bed and washed with 30% THF in DCM (500 mL). The collected filtrate was concentrated under reduced pressure and azeotroped with toluene to afford tert-butyl 3,3-dimethyl-4-(piperidin-4-ylmethyl)piperazine-1-carboxylate (1.3 g) as an off-white solid which was directly used for the next step without further purification.

LCMS (ESI): m/z=312.24 [M+H]⁺.

### Preparation of tert-butyl 4-((1-(3-(2,6-bis(benzyloxy)pyridin-3-yl)-1-methyl-1H-indazol-7-yl)piperidin-4-yl)methyl)-3,3-dimethylpiperazine-1-carboxylate

To a solution of tert-butyl 3,3-dimethyl-4-(piperidin-4-ylmethyl)piperazine-1-carboxylate (1.2 g, 3.85 mmol) and 3-(2,6-bis(benzyloxy)pyridin-3-yl)-7-bromo-1-methyl-1H-indazole (1.15 g, 2.31 mmol) in 1,4-dioxane (24 mL) was added cesium carbonate (3.77 g, 11.55 mmol) and Pd-PEPPSI-1-Hept-Cl (0.18 g, 0.19 mmol) under argon. The reaction mixture was stirred at 100° C. for 16 h. The reaction mixture was diluted with EtOAc (20 mL), filtered through a celite bed, and washed with ethyl acetate (100 mL). The collected filtrate was concentrated under reduced pressure to provide a crude product that was purified by flash column chromatography (SiO₂, 100-200 mesh, 0-30% ethyl acetate in petroleum ether) to afford tert-butyl 4-((1-(3-(2,6-bis(benzyloxy)pyridin-3-yl)-1-methyl-1H-indazol-7-yl)piperidin-4-yl)methyl)-3,3-dimethylpiperazine-1-carboxylate (1.0 g) as a brown semi-solid.

LC-MS (ESI): m/z=731.50 [M+H]⁺.

### Preparation of tert-butyl 4-((1-(3-(2,6-dioxopiperidin-3-yl)-1-methyl-1H-indazol-7-yl)piperidin-4-yl)methyl)-3,3-dimethylpiperazine-1-carboxylate

To a solution of tert-butyl 4-((1-(3-(2,6-bis(benzyloxy)pyridin-3-yl)-1-methyl-1H-indazol-7-yl)piperidin-4-yl)methyl)-3,3-dimethylpiperazine-1-carboxylate (1.0 g, 1.36 mmol) in THF (30.0 mL) was added 20% Pd(OH)₂ on carbon, moisture 50% wet (2.0 g). The reaction mixture was stirred under hydrogen pressure (80 Psi) at room temperature for 16 h. The reaction mixture was diluted with DCM (50 mL), filtered through a celite bed, and washed with 30% THF in DCM (100 mL). The collected filtrate was concentrated under reduced pressure to provide a crude product that was triturated by using diethyl ether to afford tert-butyl 4-((1-(3-(2,6-dioxopiperidin-3-yl)-1-methyl-1H-indazol-7-yl)piperidin-4-yl)methyl)-3,3-dimethylpiperazine-1-carboxylate (0.8 g) as an off white solid.

LC-MS (ESI): m/z=553.36 [M+H]⁺.

### Preparation of 3-(7-(4-((2,2-dimethylpiperazin-1-yl)methyl)piperidin-1-yl)-1-methyl-1H-indazol-3-yl)piperidine-2,6-dione

To a solution of tert-butyl 4-((1-(3-(2,6-dioxopiperidin-3-yl)-1-methyl-1H-indazol-7-yl)piperidin-4-yl)methyl)-3,3-dimethylpiperazine-1-carboxylate (0.7 g, 1.26 mmol) in DCM (7.0 mL) was added TFA (3.5 mL). The reaction mixture was stirred at room temperature for 1 h. The reaction mixture was concentrated and co-distilled with petroleum ether under reduced pressure to provide a crude product that was triturated using diethyl ether to afford 3-(7-(4-((2,2-dimethylpiperazin-1-yl)methyl)piperidin-1-yl)-1-methyl-1H-indazol-3-yl)piperidine-2,6-dione (0.7 g).

LC-MS (ESI): m/z=453.36 [M+H]⁺

### Preparation of 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,2-dimethylpiperazin-1-yl)methyl)piperidin-1-yl)-1-methyl-1H-indazol-3-yl)piperidine-2,6-dione

To a suspension of 3-(7-(4-((2,2-dimethylpiperazin-1-yl)methyl)piperidin-1-yl)-1-methyl-1H-indazol-3-yl)piperidine-2,6-dione (0.15 g, 0.33 mmol) in DMSO (3 mL), N,N-diisopropylethylamine (0.46 mL, 2.64 mol) and (S)-2-cyclopropyl-10-((2,5-dichloropyrimidin-4-yl)amino)-3,3-difluoro-7-methyl-1,2,3,4-tetrahydro-[1,4]oxazepino [2,3-c]quinolin-6(7H)-one (0.092 g, 0.19 mmol) were added. The reaction mixture was stirred at 110° C. for 16 h. The reaction was concentrated under reduce pressure to provide a crude product that was purified by prep-HPLC to afford 3-(7-(4-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,2-dimethylpiperazin-1-yl)methyl)piperidin-1-yl)-1-methyl-1H-indazol-3-yl)piperidine-2,6-dione (0.046 g) as an off white solid.

LC-MS (ESI): m/z=884.56 [M+H]⁺.

¹H NMR (400 MHz, DMSO-d6): δ 10.87 (bs, 1H), 8.81 (s, 1H), 8.19-8.15 (m, 2H), 8.01 (bs, 1H), 7.71 (dd, J=6.8, 2.8 Hz, 1H), 7.44 (d, J=9.2 Hz, 1H), 7.36 (dd, J=2.8, 0.44 Hz, 1H) 7.02-6.99 (m, 2H), 6.19 (bs, 1H), 4.44-4.30 (m, 3H), 4.23 (s, 3H), 3.60-3.59 (m, 5H), 3.26-3.22 (m, 3H), 2.65-2.59 (m, 5H), 2.39-2.31 (m, 1H), 2.22-2.15 (m, 2H), 1.87-1.86 (m, 2H), 1.60 (s, 1H), 1.35-1.29 (m, 5H), 1.14 (s, 1H), 0.92 (br s, 6H), 0.79-0.73 (m, 1H), 0.55-0.51 (m, 2H), 0.34-0.32 (m, 1H).

### Example 134: 3-(7-(4-(((1R, 4R)-5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-diazabicyclo[2.2.1]heptan-2-yl)methyl)piperidin-1-yl)-1-methyl-1H-indazol-3-yl)piperidine-2,6-dione (Compound 481a)

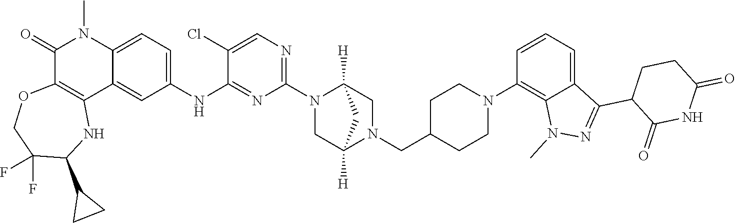 [SMILES: Cn1nc(C2CCC(=O)NC2=O)c2cccc(N3CCC(CN4C[C@H]5C[C@@H]4CN5c4ncc(Cl)c(Nc5ccc6c(c5)c5c(c(=O)n6C)OCC(F)(F)[C@H](C6CC6)N5)n4)CC3)c21]

### Preparation of tert-butyl-(1R, 4R)-5-((1-((benzyloxy)carbonyl)piperidin-4-yl)methyl)-2,5-diazabicyclo[2.2.1]heptane-2-carboxylate

To a solution of tert-butyl (1R, 4R)-2,5-diazabicyclo[2.2.1]heptane-2-carboxylate (3.0 g, 15.15 mmol) and benzyl 4-formylpiperidine-1-carboxylate (3.74 g, 15.15 mmol) in DCM (60 mL) was added acetic acid (0.91 ml, 15.15 mmol). The reaction mixture was stirred for 4 h. Sodium triacetoxyborohydride (3.83 g, 18.18 mmol) was then added under nitrogen atmosphere, and the reaction mixture was stirred at room temperature for 8 h. The reaction mixture was quenched with water (200 mL) and extracted with ethyl acetate (3×200 mL). The combined organic layers were washed with brine, dried over anhydrous Na₂SO₄, filtered, and concentrated under reduced pressure to provide a crude product that was purified by flash column chromatography (SiO₂, 230-400, 30% ethyl acetate in petroleum ether) to afford tert-butyl-(1R, 4R)-5-((1-((benzyloxy)carbonyl)piperidin-4-yl)methyl)-2,5-diazabicyclo[2.2.1]heptane-2-carboxylate (4.10 g) as an off white solid.

LC-MS (ESI): m/z=430.52 [M+H]⁺

### Preparation of tert-butyl (1R, 4R)-5-(piperidin-4-ylmethyl)-2,5-diazabicyclo[2.2.1]heptane-2-carboxylate

To a solution of tert-butyl-(1R, 4R)-5-((1-((benzyloxy)carbonyl)piperidin-4-yl)methyl)-2,5-diazabicyclo[2.2.1]heptane-2-carboxylate (4.10 g, 9.55 mmol) in THF (82 mL) was added 10% Pd/C, H₂ (2.0 g, 50% w/w) under argon. The reaction mixture was stirred under hydrogen pressure (80 psi) at room temperature for 12 h. The reaction mixture was diluted with THF (50 mL), filtered through a celite bed, and washed with THF:DCM (1:1, 100 mL). The filtrate was concentrated and dried to provide tert-butyl (1R, 4R)-5-(piperidin-4-ylmethyl)-2,5-diazabicyclo[2.2.1]heptane-2-carboxylate (1.50 g) as a brown semi-solid which was used in the next step without further purification.

LC-MS (ESI): m/z=296.21 [M+H]⁺

### Preparation of tert-butyl (1R, 4R)-5-((1-(3-(2,6-bis(benzyloxy)pyridin-3-yl)-1-methyl-1H-indazol-7-yl)piperidin-4-yl)methyl)-2,5-diazabicyclo[2.2.1]heptane-2-carboxylate

To a solution of 3-(2,6-bis(benzyloxy)pyridin-3-yl)-7-bromo-1-methyl-1H-indazole (1.5 g, 3.00 mmol) and tert-butyl (1R, 4R)-5-(piperidin-4-ylmethyl)-2,5-diazabicyclo[2.2.1]heptane-2-carboxylate (1.06 g, 3.60 mmol) in 1,4-dioxane (30 mL) was added cesium carbonate (2.92 g, 9.00 mmol) under argon followed by the addition of Pd-PEPPSI-iHept-Cl (0.14 g, 0.15 mmol). The reaction mixture was stirred at 100° C. for 12 h. The reaction mixture was cooled to room temperature and filtered through a celite bed. The filtrate was concentrated to provide a crude product that was purified by flash column chromatography (SiO₂, 230-400, 30% ethyl acetate in petroleum ether) to afford tert-butyl (1R, 4R)-5-((1-(3-(2,6-bis(benzyloxy)pyridin-3-yl)-1-methyl-1H-indazol-7-yl)piperidin-4-yl)methyl)-2,5-diazabicyclo[2.2.1]heptane-2-carboxylate (1.60 g) as an off white solid.

LC-MS (ESI): m/z=715.46 [M+H]⁺

### Preparation of tert-butyl (1R, 4R)-5-((1-(3-(2,6-dioxopiperidin-3-yl)-1-methyl-1H-indazol-7-yl)piperidin-4-yl)methyl)-2,5-diazabicyclo[2.2.1]heptane-2-carboxylate

To a solution of tert-butyl (1R, 4R)-5-((1-(3-(2,6-bis(benzyloxy)pyridin-3-yl)-1-methyl-1H-indazol-7-yl)piperidin-4-yl)methyl)-2,5-diazabicyclo[2.2.1]heptane-2-carboxylate (1.60 g, 2.24 mmol) in THF (32 mL) was added 20% Pd(OH)₂ on carbon (1.60 g) at rt. The reaction mixture was stirred under hydrogen pressure (80 psi) at room temperature for 12 h. The reaction mixture was diluted with THF (100 mL), filtered through a celite bed, and washed with THF:DCM (1:1, 200 mL). The filtrate was concentrated and dried to afford tert-butyl (1R, 4R)-5-((1-(3-(2,6-dioxopiperidin-3-yl)-1-methyl-1H-indazol-7-yl)piperidin-4-yl)methyl)-2,5-diazabicyclo[2.2.1]heptane-2-carboxylate (1.20 g) as a brown semi-solid which was used in the next step without further purification.

LC-MS (ESI): m/z=537.66 [M+H]⁺.

### Preparation of 3-(7-(4-(((1R, 4R)-2,5-diazabicyclo[2.2.1]heptan-2-yl)methyl)piperidin-1-yl)-1-methyl-1H-indazol-3-yl)piperidine-2,6-dione

To a solution of tert-butyl (1R, 4R)-5-((1-(3-(2,6-dioxopiperidin-3-yl)-1-methyl-1H-indazol-7-yl)piperidin-4-yl)methyl)-2,5-diazabicyclo[2.2.1]heptane-2-carboxylate (1.20 g, 2.23 mmol) in DCM (24 mL) was added TFA (12 mL) at 0° C. The reaction mixture was stirred at room temperature for 3 h. The reaction mixture was concentrated to provide a crude product that was triturated with diethyl ether (3 times) and dried to afford 3-(7-(4-(((1R, 4R)-2,5-diazabicyclo[2.2.1]heptan-2-yl)methyl)piperidin-1-yl)-1-methyl-1H-indazol-3-yl)piperidine-2,6-dione (0.70 g) as a brown solid.

LC-MS (ESI): m/z=437.55 [M+H]⁺.

### Preparation of 3-(7-(4-(((1R, 4R)-5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-diazabicyclo[2.2.1]heptan-2-yl)methyl)piperidin-1-yl)-1-methyl-1H-indazol-3-yl)piperidine-2,6-dione

To a solution of 3-(7-(4-(((1R, 4R)-2,5-diazabicyclo[2.2.1]heptan-2-yl)methyl)piperidin-1-yl)-1-methyl-1H-indazol-3-yl)piperidine-2,6-dione (0.25 g, 0.57 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.22 mmol) in DMSO (5.0 mL) was added N,N-diisopropylethylamine (0.79 mL, 4.56 mmol). The resulting reaction mixture was stirred at 100° C. for 12 h. The reaction mixture was added to crushed ice and stirred for 30 min to precipitate a solid. The solid was filtered and dried to obtain a crude product that was purified by prep-HPLC to afford 3-(7-(4-(((1R, 4R)-5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-diazabicyclo[2.2.1]heptan-2-yl)methyl)piperidin-1-yl)-1-methyl-1H-indazol-3-yl)piperidine-2,6-dione (126 mg) as an off white solid.

LC-MS (ESI): m/z=868.52 [M+H]⁺.

¹H NMR (400 MHz, DMSO-d6): δ 10.87 (s, 1H), 8.73 (d, J=18.40 Hz, 1H), 8.26-8.16 (m, 1H), 8.02 (s, 1H), 7.82 (s, 1H), 7.44 (d, J=9.20 Hz, 1H), 7.35 (d, J=7.20 Hz, 1H), 6.98-6.96 (m, 2H), 6.18 (d, J=17.20 Hz, 1H), 4.63-4.30 (m, 4H), 4.21 (s, 3H), 3.56 (s, 5H), 3.21 (br, 4H), 2.86 (s, 1H), 2.57-2.51 (m, 5H), 2.44 (br, 2H), 2.33-2.30 (m, 1H), 2.15-2.13 (m, 1H), 1.84 (s, 3H), 1.68 (d, J=8.80 Hz, 1H), 1.45-1.34 (m, 4H), 0.72 (d, J=6.00 Hz, 1H), 0.51 (s, 2H), 0.37 (t, J=4.80 Hz, 1H).

### Example 135: 3-(7-(4-(((1S, 4S)-5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-diazabicyclo[2.2.1]heptan-2-yl)methyl)piperidin-1-yl)-1-methyl-1H-indazol-3-yl)piperidine-2,6-dione (Compound 481b)

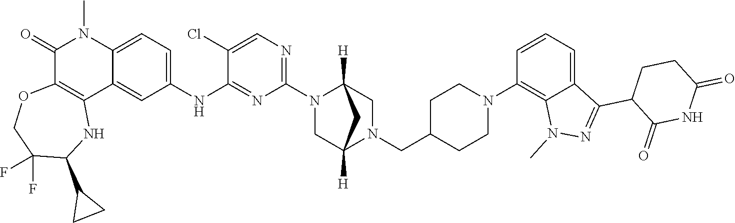 [SMILES: Cn1nc(C2CCC(=O)NC2=O)c2cccc(N3CCC(CN4C[C@@H]5C[C@H]4CN5c4ncc(Cl)c(Nc5ccc6c(c5)c5c(c(=O)n6C)OCC(F)(F)[C@H](C6CC6)N5)n4)CC3)c21]

### Preparation of tert-butyl (1S, 4S)-5-((1-((benzyloxy)carbonyl) piperidin-4-yl)methyl)-2,5-diazabicyclo[2.2.1]heptane-2-carboxylate

To a solution of tert-butyl (1S, 4S)-2,5-diazabicyclo [2.2.1]heptane-2-carboxylate (1.5 g, 7.56 mmol) and benzyl 4-formylpiperidine-1-carboxylate (1.8 g, 7.56 mmol) in dichloromethane (30 ml) was added acetic acid (0.43 mL). The reaction mixture was allowed to stir at room temperature for 2 h. Sodium triacetoxyborohydride (1.92 g, 9.07 mmol) was then added, and the reaction was stirred at room temperature for 16 h. The reaction mixture was quenched with ice-cold water (100 mL) and extracted with DCM (3×100 mL). The combined organic layers were washed with brine (100 mL), dried over anhydrous Na₂SO₄, filtered, and dried under vacuum to provide a crude product that was purified by column chromatography (SiO2; 40% EtOAc in petroleum ether) to afford tert-butyl (1S, 4S)-5-((1-((benzyloxy)carbonyl) piperidin-4-yl)methyl)-2,5-diazabicyclo[2.2.1]heptane-2-carboxylate (3.0 g) as a white solid.

LC-MS (ESI): m/z=430.32 [M+H]⁺.

### Preparation of tert-butyl (1S, 4S)-5-(piperidin-4-ylmethyl)-2,5-diazabicyclo [2.2.1]heptane-2-carboxylate

To a solution tert-butyl (1S, 4S)-5-((1-((benzyloxy)carbonyl) piperidin-4-yl)methyl)-2,5-diazabicyclo[2.2.1]heptane-2-carboxylate (3.0 g, 6.98 mmol) in THF (180 mL) was added 20% Pd(OH)₂ on carbon (3.0 g, 100% w/w). The reaction mixture was stirred under hydrogen atmosphere (60 psi) at room temperature for 8 h. The reaction mixture was filtered through a celite bed and concentrated to obtain a crude product that was washed with n-pentane and dried to afford tert-butyl (1S, 4S)-5-(piperidin-4-ylmethyl)-2,5-diazabicyclo [2.2.1]heptane-2-carboxylate (1.8 g) as a white solid.

¹H NMR (400 MHz, DMSO-d6): δ 4.11-4.14 (m, 1H), 3.38-3.40 (m, 1H), 3.27-3.29 (m, 1H), 3.15-3.04 (m, 3H), 2.77-2.78 (m, 1H), 2.60-2.61 (m, 2H), 2.30-2.32 (m, 3H), 1.87 (s, 2H), 1.59-1.62 (m, 4H), 1.48 (s, 9H).

### Preparation of tert-butyl (1S, 4S)-5-((1-(3-(2,6-bis(benzyloxy)pyridin-3-yl)-1-methyl-1H-indazol-7-yl)piperidin-4-yl)methyl)-2,5-diazabicyclo[2.2.1]heptane-2-carboxylate

A solution of 3-(2,6-bis(benzyloxy)pyridin-3-yl)-7-bromo-1-methyl-1H-indazole (1 g, 1.99 mmol), tert-butyl (1S, 4S)-5-(piperidin-4-ylmethyl)-2,5-diazabicyclo [2.2.1]heptane-2-carboxylate (1.5 g, 4.97 mmol) and cesium carbonate (1.9 g, 5.97 mmol) in DMF (20 mL) was degassed with argon for 15 minutes. Then, PEPPSI i-Hept Cl (80 mg, 0.10 mmol) was added, and the reaction mixture was stirred and heated at 140° C. for 2 h. The reaction mixture was diluted with ice-cold water (100 mL) and extracted into ethyl acetate (3×100 mL). The combined organic layers were washed with brine (100 mL), dried over anhydrous Na₂SO₄, filtered, and dried under vacuum to provide a crude product that was purified by column chromatography (SiO₂; 100-200 mesh, 40% EtOAc in petroleum ether) to afford tert-butyl (1S, 4S)-5-((1-(3-(2,6-bis(benzyloxy)pyridin-3-yl)-1-methyl-1H-indazol-7-yl)piperidin-4-yl)methyl)-2,5-diazabicyclo[2.2.1]heptane-2-carboxylate (0.8 g) as a yellow solid.

LC-MS (ESI): m/z=[M+H]⁺.

### Preparation of tert-butyl (1S, 4S)-5-((1-(3-(2,6-dioxopiperidin-3-yl)-1-methyl-1H-indazol-7-yl)piperidin-4-yl)methyl)-2,5-diazabicyclo[2.2.1]heptane-2-carboxylate

To a solution of tert-butyl (1S, 4S)-5-((1-(3-(2,6-bis(benzyloxy)pyridin-3-yl)-1-methyl-1H-indazol-7-yl)piperidin-4-yl)methyl)-2,5-diazabicyclo[2.2.1]heptane-2-carboxylate (0.8 g, 1.12 mmol) in THF (30.0 mL) was added 20% Pd (OH)₂ on carbon (0.8 g, 100% w/w). The reaction mixture was stirred under hydrogen atmosphere (60 psi) at room temperature for 6 h. The reaction mixture was filtered through a celite bed and concentrated under reduced pressure to obtain a crude product that was washed with n-pentane and dried to afford tert-butyl (1S, 4S)-5-((1-(3-(2,6-dioxopiperidin-3-yl)-1-methyl-1H-indazol-7-yl)piperidin-4-yl)methyl)-2,5-diazabicyclo[2.2.1]heptane-2-carboxylate (0.6 g) as a green solid.

LC-MS (ESI): m/z=537.63 [M+H]⁺.

### Preparation of 3-(7-(4-(((1S, 4S)-2,5-diazabicyclo[2.2.1]heptan-2-yl)methyl)piperidin-1-yl)-1-methyl-1H-indazol-3-yl)piperidine-2,6-dione

To a solution of tert-butyl (1S, 4S)-5-((1-(3-(2,6-dioxopiperidin-3-yl)-1-methyl-1H-indazol-7-yl)piperidin-4-yl)methyl)-2,5-diazabicyclo[2.2.1]heptane-2-carboxylate (0.6 g, 0.93 mmol) in DCM (5.0 mL) at 0° C. was added TFA (2.5 mL). The reaction mixture was stirred at room temperature for 3 h. The reaction mixture was concentrated under reduced pressure and co-distilled with diethyl ether to provide a crude product that was washed with n-pentane to afford 3-(7-(4-(((1S, 4S)-2,5-diazabicyclo[2.2.1]heptan-2-yl)methyl)piperidin-1-yl)-1-methyl-1H-indazol-3-yl)piperidine-2,6-dione (0.46 g) as a white solid.

LC-MS: m/z=437.45 [M+H]⁺

### Preparation of 3-(7-(4-(((1S, 4S)-5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-diazabicyclo[2.2.1]heptan-2-yl)methyl)piperidin-1-yl)-1-methyl-1H-indazol-3-yl)piperidine-2,6-dione

To a solution 3-(7-(4-(((1S, 4S)-2,5-diazabicyclo[2.2.1]heptan-2-yl)methyl)piperidin-1-yl)-1-methyl-1H-indazol-3-yl)piperidine-2,6-dione (0.20 g, 0.45 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (90 mg, 0.18 mmol) in DMSO (4.0 mL) was added N,N-diisopropylethylamine (0.4 mL, 2.29 mmol). The reaction mixture was stirred at 100° C. for 16 h.

The reaction mixture was poured into ice cold water and stirred for 15 minutes. The resulting precipitate was filtered and dried to provide a crude product that was purified by prep-HPLC to afford 3-(7-(4-(((1S, 4S)-5-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2,5-diazabicyclo[2.2.1]heptan-2-yl)methyl)piperidin-1-yl)-1-methyl-1H-indazol-3-yl)piperidine-2,6-dione (0.047 g) as a white solid.

LC-MS (ESI): m/z=868.56 [M+H]⁺.

¹H NMR (400 MHz, DMSO-d6): δ 10.90 (s, 1H), 8.75-8.69 (m, 1H), 8.24 (s, 1H), 8.02 (s, 1H), 7.87-7.75 (m, 1H), 7.40 (dd, J=8.40, 33.60 Hz, 2H), 7.00-6.98 (m, 2H), 6.18-6.11 (m, 1H), 4.32-4.30 (m, 4H), 4.28-4.22 (m, 3H), 3.56-3.47 (m, 5H), 3.24-3.22 (m, 4H), 2.85 (br s, 1H), 2.57-2.54 (m, 5H), 2.50-2.50 (m, 2H), 2.30-2.29 (m, 1H), 2.17-2.15 (m, 1H), 1.78-1.77 (m, 3H), 1.68-1.66 (m, 1H), 1.50-1.34 (m, 4H), 0.70 (bs, 1H), 0.54-0.52 (m, 2H), 0.36-0.35 (m, 1H).

### Example 136: 3-(7-((1R, 4R)-5-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2,5-diazabicyclo[2.2.1]heptan-2-yl)-1-methyl-1H-indazol-3-yl)piperidine-2,6-dione (Compound 482a)

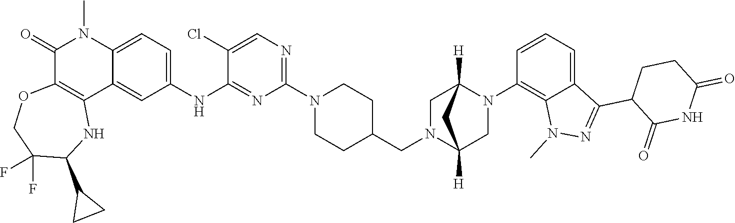 [SMILES: Cn1nc(C2CCC(=O)NC2=O)c2cccc(N3C[C@H]4C[C@@H]3CN4CC3CCN(c4ncc(Cl)c(Nc5ccc6c(c5)c5c(c(=O)n6C)OCC(F)(F)[C@H](C6CC6)N5)n4)CC3)c21]

### Preparation of tert-butyl (1R, 4R)-5-(3-(2,6-bis(benzyloxy)pyridin-3-yl)-1-methyl-1H-indazol-7-yl)-2,5-diazabicyclo[2.2.1]heptane-2-carboxylate

To a solution of 3-(2,6-bis(benzyloxy)pyridin-3-yl)-7-bromo-1-methyl-1H-indazole (2.0 g, 4.0 mmol) and tert-butyl (1R, 4R)-2,5-diazabicyclo[2.2.1]heptane-2-carboxylate (1.58 g, 8.0 mmol) in 1,4-dioxane (60.0 mL) was added cesium carbonate (3.90 g, 12.0 mmol) and Pd PEPPSI IHept Cl (0.19 g, 0.20 mmol) under argon. The reaction mixture was stirred at 100° C. for 16 h.

The reaction mixture was cooled to rt and diluted with ethyl acetate (20 mL), filtered through a celite bed, and washed with 10% MeOH:DCM (100 mL). The filtrate was concentrated to provide a crude product that was purified by flash chromatography (230-400 silica gel; 30% EtOAc in petroleum ether) to afford tert-butyl (1R, 4R)-5-(3-(2,6-bis(benzyloxy)pyridin-3-yl)-1-methyl-1H-indazol-7-yl)-2,5-diazabicyclo[2.2.1]heptane-2-carboxylate as a pale yellow solid (1.95 g).

LC-MS (ESI): m/z=618.38 [M+H]⁺

### Preparation of 5-(7-((1R, 4R)-2,5-diazabicyclo[2.2.1]heptan-2-yl)-1-methyl-1H-indazol-3-yl)-6-(benzyloxy)pyridin-2-ol

To a solution of tert-butyl (1R, 4R)-5-(3-(2,6-bis(benzyloxy)pyridin-3-yl)-1-methyl-1H-indazol-7-yl)-2,5-diazabicyclo[2.2.1]heptane-2-carboxylate (1.70 g, 2.97 mmol) in DCM (17.0 mL, 10 V) was added TFA (12.0 mL) at 0° C. The reaction mixture was stirred at room temperature for 3 h.

The reaction mixture was concentrated under reduced pressure and co-distilled with DCM (2×20 mL) to provide a crude product that was triturated with diethyl ether (2×10 mL). The solvent was decanted and dried to afford 5-(7-((1R, 4R)-2,5-diazabicyclo[2.2.1]heptan-2-yl)-1-methyl-1H-indazol-3-yl)-6-(benzyloxy)pyridin-2-ol (2.10 g) as a pale yellow gummy compound.

LC-MS (ESI): m/z=428.46 [M+H]⁺

### Preparation of benzyl 4-(((1R, 4R)-5-(3-(2-(benzyloxy)-6-hydroxypyridin-3-yl)-1-methyl-1H-indazol-7-yl)-2,5-diazabicyclo[2.2.1]heptan-2-yl)methyl)piperidine-1-carboxylate

To a solution of 5-(7-((1R, 4R)-2,5-diazabicyclo[2.2.1]heptan-2-yl)-1-methyl-1H-indazol-3-yl)-6-(benzyloxy)pyridin-2-ol (2.0 g, 4.67 mmol) and benzyl 4-formylpiperidine-1-carboxylate (1.15 g, 4.67 mmol) in THF (40.0 mL) was added sodium acetate (1.15 g, 14.03 mmol) and acetic acid (2.0 mL). The reaction mixture was stirred at rt for 1 h. Then was added sodium triacetoxyborohydride (3.0 g, 14.03 mmol) at 0° C., and the resultant reaction mixture was stirred at room temperature for 16 h. The reaction mixture was quenched with NH₄Cl solution (30 mL) and extracted with DCM (30 mL×2). The combined organic layer was dried over anhydrous Na₂SO₄, filtered, and concentrated to provide a crude product that was purified by flash chromatography (230-400 silica gel; 6% MeOH in DCM) to afford benzyl 4-(((1R, 4R)-5-(3-(2-(benzyloxy)-6-hydroxypyridin-3-yl)-1-methyl-1H-indazol-7-yl)-2,5-diazabicyclo[2.2.1]heptan-2-yl)methyl)piperidine-1-carboxylate as a pale yellow solid (0.90 g).

LC-MS (ESI): m/z=659.45 [M+H]⁺

### Preparation of 3-(1-methyl-7-((1R, 4R)-5-(piperidin-4-ylmethyl)-2,5-diazabicyclo[2.2.1]heptan-2-yl)-1H-indazol-3-yl)piperidine-2,6-dione

To a solution of benzyl 4-(((1R, 4R)-5-(3-(2-(benzyloxy)-6-hydroxypyridin-3-yl)-1-methyl-1H-indazol-7-yl)-2,5-diazabicyclo[2.2.1]heptan-2-yl)methyl)piperidine-1-carboxylate (0.90 g, 1.36 mmol) in THF (36.0 mL) was added acetic acid (0.9 mL) followed by 20% Pd(OH)₂ on carbon (1.80 g, 200% w/w). The reaction mixture was stirred under hydrogen atmosphere (85 psi) at room temperature for 16 h. The reaction mixture was diluted with THF (30 mL), filtered through a celite bed, and washed with THF:DCM (1:1, 200 mL). The filtrate was concentrated and dried to provide a crude product that was dissolved in acetone (3 mL) and reprecipitated with diethyl ether (10 mL) to afford 3-(1-methyl-7-((1R, 4R)-5-(piperidin-4-ylmethyl)-2,5-diazabicyclo[2.2.1]heptan-2-yl)-1H-indazol-3-yl)piperidine-2,6-dione (0.21 g).

LC-MS (ESI): m/z=437.50 [M+H]⁺

### Preparation of 3-(7-((1R, 4R)-5-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2,5-diazabicyclo[2.2.1]heptan-2-yl)-1-methyl-1H-indazol-3-yl)piperidine-2,6-dione

To a stirred solution of 3-(1-methyl-7-((1R, 4R)-5-(piperidin-4-ylmethyl)-2,5-diazabicyclo[2.2.1]heptan-2-yl)-1H-indazol-3-yl)piperidine-2,6-dione (0.09 g, 0.21 mmol) in DMSO (0.90 mL) was added DIPEA (0.9 mL) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.024 g, 0.05 mmol). The resulting reaction mixture was stirred at 110° C. for 16 h. The reaction mixture was added to cold water and the precipitated solid was filtered, dried, and purified by prep-HPLC to afford 3-(7-((1R, 4R)-5-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2,5-diazabicyclo[2.2.1]heptan-2-yl)-1-methyl-1H-indazol-3-yl)piperidine-2,6-dione (0.012 g) as an off white solid.

LC-MS (ESI): 868.52 [M+H]⁺.

¹H NMR (400 MHz, DMSO-d6): δ 10.87 (s, 1H), 8.77 (s, 1H), 8.32 (s, 1H), 8.02 (s, 1H), 7.74 (d, J=7.60 Hz, 1H), 7.44 (d, J=9.20 Hz, 1H), 7.29 (d, J=7.60 Hz, 1H), 7.05-6.97 (m, 2H), 6.16 (s, 1H), 4.45-4.30 (m, 5H), 4.21 (s, 3H), 3.88 (s, 1H), 3.57 (s, 4H), 3.24-3.20 (m, 1H), 3.05 (t, J=8.40 Hz, 1H), 2.80-2.54 (m, 8H), 2.41-2.39 (m, 1H), 2.36-2.33 (m, 1H), 2.19-2.13 (m, 1H), 1.88-1.75 (m, 4H), 1.58 (br, 1H), 1.35 (s, 1H), 1.08-1.01 (m, 2H), 0.74-0.68 (m, 1H), 0.52 (t, J=6.00 Hz, 2H), 0.36-0.34 (m, 1H).

### Example 137: 3-(7-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,6-diazabicyclo[3.1.1]heptan-3-yl)methyl)piperidin-1-yl)-1-methyl-1H-indazol-3-yl)piperidine-2,6-dione (Compound 483a)

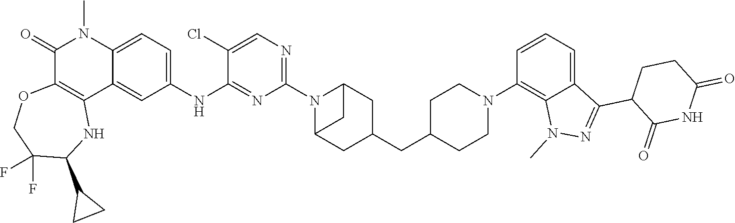 [SMILES: Cn1nc(C2CCC(=O)NC2=O)c2cccc(N3CCC(CC4CC5CC(C4)N5c4ncc(Cl)c(Nc5ccc6c(c5)c5c(c(=O)n6C)OCC(F)(F)[C@H](C6CC6)N5)n4)CC3)c21]

### Preparation of tert-butyl 3-((1-((benzyloxy)carbonyl)piperidin-4-yl)methyl)-3,6-diazabicyclo[3.1.1]heptane-6-carboxylate

To a solution of tert-butyl 3,6-diazabicyclo[3.1.1]heptane-6-carboxylate (2.0 g, 10.08 mmol) and benzyl 4-formylpiperidine-1-carboxylate (2.99 g, 12.10 mmol) in DCM (40 mL) was added acetic acid (2.0 mL). The reaction mixture was stirred for 30 min. Sodium triacetoxyborohydride (2.13 g, 10.08 mmol) was then added at 0° C. under nitrogen atmosphere. The reaction mixture was stirred at room temperature for 16 h. The reaction mixture was diluted with water (200 mL) and extracted with ethyl acetate (2×100 mL). The combined organic layers were washed with brine, dried over anhydrous Na₂SO₄, filtered, and concentrated under reduced pressure to provide a crude product that was purified by flash column chromatography (SiO₂, 60-120 mesh, 25% ethyl acetate in petroleum ether) to afford tert-butyl 3-((1-((benzyloxy)carbonyl)piperidin-4-yl)methyl)-3,6-diazabicyclo[3.1.1]heptane-6-carboxylate (3.5 g) as a yellow semi-solid.

LC-MS (ESI): m/z=430.52 [M+H]⁺.

### Preparation of tert-butyl 3-(piperidin-4-ylmethyl)-3,6-diazabicyclo[3.1.1]heptane-6-carboxylate

To a solution of tert-butyl 3-((1-((benzyloxy)carbonyl)piperidin-4-yl)methyl)-3,6-diazabicyclo[3.1.1]heptane-6-carboxylate (2.5 g, 5.81 mmol) in THF (75 mL) was added 20% Pd (OH)₂ on carbon, 50% wet (2.5 g, 100% w/w). The reaction mixture was stirred under hydrogen atmosphere (80 psi) at room temperature for 16 h. The reaction mixture was diluted with DCM (50 mL), filtered through a celite bed, and washed with 30% THF in DCM (500 mL). The collected filtrate was concentrated under reduced pressure and azeotroped with toluene to provide tert-butyl 3-(piperidin-4-ylmethyl)-3,6-diazabicyclo[3.1.1]heptane-6-carboxylate (1.5 g) as an off-white solid which was directly used in the next step without further purification.

LC-MS (ESI): m/z=296.56 [M+H]⁺.

### Preparation of tert-butyl 3-((1-(3-(2,6-bis(benzyloxy)pyridin-3-yl)-1-methyl-1H-indazol-7-yl)piperidin-4-yl)methyl)-3,6-diazabicyclo[3.1.1]heptane-6-carboxylate

To a solution of 3-(2,6-bis(benzyloxy)pyridin-3-yl)-7-bromo-1-methyl-1H-indazole (0.9 g, 1.79 mmol), tert-butyl 3-(piperidin-4-ylmethyl)-3,6-diazabicyclo[3.1.1]heptane-6-carboxylate (1.68 g, 5.37 mmol) in 1,4-dioxane (18 mL) was added cesium carbonate (1.74 g, 5.37 mmol) under argon. Pd-PEPPSI-IHeptCl (0.08 g, 0.08 mmol) was then added, and the reaction mixture was stirred at 100° C. for 16 h. The reaction mixture was diluted with EtOAc (20 mL), filtered through a celite bed, and washed with ethyl acetate (100 mL). The collected filtrate was concentrated under reduced pressure to provide a crude product that was purified by flash column chromatography (SiO₂, 100-200 mesh, 50% ethyl acetate in petroleum ether) to afford tert-butyl 3-((1-(3-(2,6-bis(benzyloxy)pyridin-3-yl)-1-methyl-1H-indazol-7-yl)piperidin-4-yl)methyl)-3,6-diazabicyclo[3.1.1]heptane-6-carboxylate (0.6 g) as a brown semi-solid.

LC-MS (ESI): m/z=715.46 [M+H]⁺.

### Preparation of tert-butyl 3-((1-(3-(2,6-dioxopiperidin-3-yl)-1-methyl-1H-indazol-7-yl)piperidin-4-yl)methyl)-3,6-diazabicyclo[3.1.1]heptane-6-carboxylate

To a solution of tert-butyl 3-((1-(3-(2,6-bis(benzyloxy)pyridin-3-yl)-1-methyl-1H-indazol-7-yl)piperidin-4-yl)methyl)-3,6-diazabicyclo[3.1.1]heptane-6-carboxylate (0.60 g, 0.83 mmol) in THF (18.0 mL) was added 20% Pd (OH)₂ on carbon (0.6 g). The reaction mixture was stirred under hydrogen atmosphere (80 Psi) at room temperature for 18 h. The reaction mixture was diluted with DCM (50 mL), filtered through a celite bed, and washed with 30% THF in DCM (100 mL).

The collected filtrate was concentrated under reduced pressure to provide a crude product that was triturated by using diethyl ether to afford tert-butyl 3-((1-(3-(2,6-dioxopiperidin-3-yl)-1-methyl-1H-indazol-7-yl)piperidin-4-yl)methyl)-3,6-diazabicyclo[3.1.1]heptane-6-carboxylate (0.50 g) as an off white solid.

LC-MS (ESI): m/z=537.66 [M+H]⁺.

### Preparation of 3-(7-(4-((3,6-diazabicyclo[3.1.1]heptan-3-yl)methyl)piperidin-1-yl)-1-methyl-1H-indazol-3-yl)piperidine-2,6-dione

To a solution of tert-butyl 3-((1-(3-(2,6-dioxopiperidin-3-yl)-1-methyl-1H-indazol-7-yl)piperidin-4-yl)methyl)-3,6-diazabicyclo[3.1.1]heptane-6-carboxylate (0.50 g, 0.93 mmol) in DCM (5.0 mL) was added TFA (5.0 mL). The reaction mixture was stirred at room temperature for 1 h. The reaction mixture was concentrated and co-distilled with petroleum ether under reduced pressure to provide a crude product that was triturated with diethyl ether to afford 3-(7-(4-((3,6-diazabicyclo[3.1.1]heptan-3-yl)methyl)piperidin-1-yl)-1-methyl-1H-indazol-3-yl)piperidine-2,6-dione (0.42 g).

LC-MS (ESI): m/z=437.29 [M+H]⁺.

### Preparation of 3-(7-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,6-diazabicyclo[3.1.1]heptan-3-yl)methyl)piperidin-1-yl)-1-methyl-1H-indazol-3-yl)piperidine-2,6-dione

To a solution of 3-(7-(4-((3,6-diazabicyclo[3.1.1]heptan-3-yl)methyl)piperidin-1-yl)-1-methyl-1H-indazol-3-yl)piperidine-2,6-dione (0.2 g, 0.45 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.13 mmol) in DMSO (4 mL) was added N,N-diisopropylethylamine (0.29 g, 2.25 mmol). The reaction mixture was stirred at 100° C. for 16 h.

The reaction mixture was poured into ice-cold water and stirred for 15 minutes. The resulting precipitate was filtered and dried to provide a crude product that was purified by prep-HPLC to afford 3-(7-(4-((6-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,6-diazabicyclo[3.1.1]heptan-3-yl)methyl)piperidin-1-yl)-1-methyl-1H-indazol-3-yl)piperidine-2,6-dione (0.05 g) as a white solid.

LC-MS (ESI): m/z=868.52 [M+H]⁺.

¹H NMR (400 MHz, DMSO-d6): δ 10.88 (s, 1H), 8.75 (s, 1H), 8.16-8.06 (m, 2H), 7.84 (br s, 1H), 7.45 (d, J=9.20 Hz, 1H), 7.35 (d, J=7.60 Hz, 1H), 6.96 (d, J=23.60 Hz, 2H), 6.03 (s, 1H), 4.42-4.41 (m, 3H), 4.32-4.31 (m, 5H), 3.56 (s, 3H), 3.17-3.14 (m, 5H), 2.59-2.57 (m, 4H), 2.44 (br, 2H), 2.30-2.29 (m, 3H), 2.15-2.13 (m, 1H), 1.55 (d, J=7.60 Hz, 4H), 1.24-1.17 (m, 5H), 0.71-0.69 (m, 1H), 1.17 (br, 2H), 0.35 (br, 1H).

### Example 138: 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperazin-1-yl)methyl)piperidin-1-yl)-1-methyl-1H-indazol-3-yl)piperidine-2,6-dione (Compound 476b)

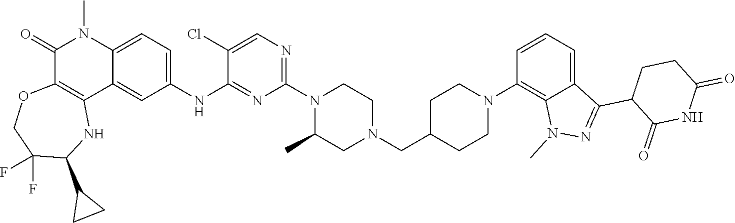 [SMILES: C[C@@H]1CN(CC2CCN(c3cccc4c(C5CCC(=O)NC5=O)nn(C)c34)CC2)CCN1c1ncc(Cl)c(Nc2ccc3c(c2)c2c(c(=O)n3C)OCC(F)(F)[C@H](C3CC3)N2)n1]

### Preparation of tert-butyl (R)-4-((1-((benzyloxy)carbonyl)piperidin-4-yl)methyl)-2-methylpiperazine-1-carboxylate

To a solution of tert-butyl (R)-2-methylpiperazine-1-carboxylate (2.0 g, 9.98 mmol) and benzyl 4-formylpiperidine-1-carboxylate (2.46 g, 9.98 mmol) in DCM (40 mL) was added acetic acid (0.57 mL, 9.98 mmol). The reaction mixture was stirred for 3 h. Afterwards, sodium triacetoxyborohydride (4.23 g, 19.96 mmol) was added, and the resulting mixture was stirred at room temperature for 16 h. The reaction mixture was quenched with ammonium chloride solution (50 mL) and extracted with DCM (2×60 mL). The combined organic layer was washed with brine (60 mL), dried over anhydrous Na₂SO₄, filtered, and concentrated under reduced pressure to provide a crude product tert-butyl (R)-4-((1-((benzyloxy)carbonyl)piperidin-4-yl)methyl)-2-methylpiperazine-1-carboxylate (3.7 g) as a pale yellow sticky compound that was used in the next step without further purification.

L CMS (ESI): m/z=432.54 [M+H]⁺.

### Preparation of tert-butyl (R)-2-methyl-4-(piperidin-4-ylmethyl)piperazine-1-carboxylate

To a solution of tert-butyl (R)-4-((1-((benzyloxy)carbonyl)piperidin-4-yl)methyl)-2-methylpiperazine-1-carboxylate (2.0 g, 4.63 mmol) in THF (60 mL) was added 20% Pd(OH)₂ on carbon (2.0 g, 100% w/w). The reaction mixture was stirred under hydrogen atmosphere (80 psi) at room temperature for 5 h. The reaction mixture was diluted with THF (50 mL), filtered through a celite bed, and washed with 200 mL of THF:DCM (1:1). The filtrate was concentrated and dried to afford tert-butyl (R)-2-methyl-4-(piperidin-4-ylmethyl)piperazine-1-carboxylate as a pale yellow gummy compound (1.35 g).

L CMS (ESI): 298.24 [M+H]⁺.

### Preparation of tert-butyl (R)-4-((1-(3-(2,6-bis(benzyloxy)pyridin-3-yl)-1-methyl-1H-indazol-7-yl)piperidin-4-yl)methyl)-2-methylpiperazine-1-carboxylate

To a solution of 3-(2,6-bis(benzyloxy)pyridin-3-yl)-7-bromo-1-methyl-1H-indazole (0.70 g, 1.39 mmol) and tert-butyl (R)-2-methyl-4-(piperidin-4-ylmethyl)piperazine-1-carboxylate (1.04 g, 3.47 mmol) in 1,4-dioxane (14.0 mL) was added cesium carbonate (1.35 g, 4.17 mmol) under argon. Pd-PEPPSI-IHeptCl (0.068 g, 0.07 mmol) was added, and the reaction mixture was stirred at 100° C. for 16 h. The reaction mixture was diluted with ethyl acetate (60 mL), filtered through a celite bed, and washed with 10% MeOH:DCM (200 mL). The filtrate was concentrated and dried under vacuum to provide a crude product that was purified by flash chromatography (230-400 silica gel; 10-15% EtOAc in petroleum ether) to afford tert-butyl (R)-4-((1-(3-(2,6-bis(benzyloxy)pyridin-3-yl)-1-methyl-1H-indazol-7-yl)piperidin-4-yl)methyl)-2-methylpiperazine-1-carboxylate (0.80 g) as a yellow sticky compound.

LC-MS (ESI): m/z=717.79 [M+H]⁺.

### Preparation of tert-butyl (2R)-4-((1-(3-(2,6-dioxopiperidin-3-yl)-1-methyl-1H-indazol-7-yl)piperidin-4-yl)methyl)-2-methylpiperazine-1-carboxylate

To a solution of tert-butyl (R)-4-((1-(3-(2,6-bis(benzyloxy)pyridin-3-yl)-1-methyl-1H-indazol-7-yl)piperidin-4-yl)methyl)-2-methylpiperazine-1-carboxylate (0.80 g, 1.11 mmol) in THF (20.0 mL) was added 20% Pd(OH)₂ on carbon (0.80 g, 100% w/w). The reaction mixture was stirred under hydrogen atmosphere (80 Psi) at room temperature for 5 h. The reaction mixture was diluted with THF (50 mL), filtered through a celite bed, and washed with THF:DCM (1:1, 200 mL). The filtrate was concentrated and dried to afford tert-butyl (2R)-4-((1-(3-(2,6-dioxopiperidin-3-yl)-1-methyl-1H-indazol-7-yl)piperidin-4-yl)methyl)-2-methylpiperazine-1-carboxylate as a pale brown solid (0.62 g) that was used in the next step without further purification.

LC-MS (ESI): m/z=539.44 [M+H]⁺.

### Preparation of 3-(1-methyl-7-(4-(((R)-3-methylpiperazin-1-yl)methyl)piperidin-1-yl)-1H-indazol-3-yl)piperidine-2,6-dione

To a stirred solution of tert-butyl (2R)-4-((1-(3-(2,6-dioxopiperidin-3-yl)-1-methyl-1H-indazol-7-yl)piperidin-4-yl)methyl)-2-methylpiperazine-1-carboxylate (0.60 g, 1.11 mmol) in DCM (6.0 mL) was added TFA (3.0 mL, 5 V) at 0° C. The reaction mixture was stirred at room temperature for 3 h. The reaction mixture was concentrated under reduced pressure, co-distilled with DCM (3×10 mL), and dried to afford 3-(1-methyl-7-(4-(((R)-3-methylpiperazin-1-yl)methyl)piperidin-1-yl)-1H-indazol-3-yl)piperidine-2,6-dione (0.35 g) as an off white solid which was used in the next step without further purification.

LC-MS (ESI): m/z=439.28 [M+H]⁺.

### Preparation of 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperazin-1-yl)methyl)piperidin-1-yl)-1-methyl-1H-indazol-3-yl)piperidine-2,6-dione

To a solution of 3-(1-methyl-7-(4-(((R)-3-methylpiperazin-1-yl)methyl)piperidin-1-yl)-1H-indazol-3-yl)piperidine-2,6-dione (0.15 g, 0.34 mmol) in DMSO (1.5 mL) and DIPEA (1.5 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.032 g, 0.07 mmol). The reaction mixture was stirred at 120° C. for 16 h. The reaction mixture was quenched with cold water. The resulting solid was filtered and dried to provide a crude product that was purified by prep-HPLC to afford 3-(7-(4-(((R)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperazin-1-yl)methyl)piperidin-1-yl)-1-methyl-1H-indazol-3-yl)piperidine-2,6-dione (0.015 g) as a pale brown solid.

LC-MS (ESI): m/z=870.50 [M+H]⁺.

¹H NMR (400 MHz, DMSO-d6): δ 10.88 (s, 1H), 8.82 (s, 1H), 8.51 (s, 1H), 8.05 (s, 1H), 7.75 (d, J=9.20 Hz, 1H), 7.44 (d, J=9.20 Hz, 1H), 7.38-7.35 (m, 1H), 7.01-7.00 (m, 2H), 6.20 (s, 1H), 4.47-4.41 (m, 4H), 4.23-4.21 (m, 4H), 3.58 (s, 3H), 3.33-3.23 (br, 3H), 3.02 (t, J=10.00 Hz, 1H), 2.83 (d, J=10.00 Hz, 1H), 2.72-2.62 (m, 5H), 2.33 (s, 1H), 2.18-2.17 (m, 3H), 2.04 (d, J=7.60 Hz, 1H), 1.87 (d, J=12.40 Hz, 3H), 1.69 (br, 1H), 1.40-1.34 (m, 3H), 1.16 (d, J=6.40 Hz, 3H), 0.75-0.64 (m, 1H), 0.57-0.43 (m, 2H), 0.30-0.38 (m, 1H).

### Example 139: 3-(7-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2-methylpiperazin-1-yl)-1-methyl-1H-indazol-3-yl)piperidine-2,6-dione (Compound 475b)

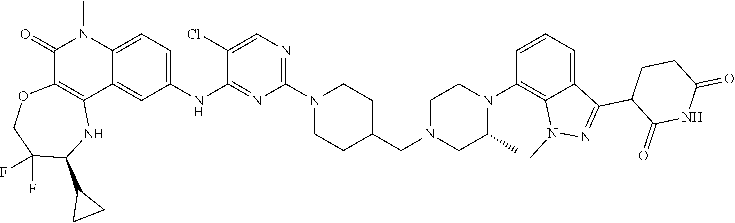 [SMILES: C[C@@H]1CN(CC2CCN(c3ncc(Cl)c(Nc4ccc5c(c4)c4c(c(=O)n5C)OCC(F)(F)[C@H](C5CC5)N4)n3)CC2)CCN1c1cccc2c(C3CCC(=O)NC3=O)nn(C)c12]

### Preparation of tert-butyl (R)-4-(3-(2,6-bis(benzyloxy)pyridin-3-yl)-1-methyl-1H-indazol-7-yl)-3-methylpiperazine-1-carboxylate

To a solution of 3-(2,6-bis(benzyloxy)pyridin-3-yl)-7-bromo-1-methyl-1H-indazole (2.0 g, 3.99 mmol) and (R)-3-methylpiperazine-1-carboxylate (3.19 g, 15.96 mmol) in 1,4-dioxane (40 mL) was added cesium carbonate (3.89 g, 11.97 mmol). The reaction mixture was degassed with argon for 10 minutes. Pd-PEPPSI-iHeptCl (0.19 g, 0.19 mmol) was then added, and the resulting mixture was again degassed for 5 min. The reaction mixture was stirred at 100° C. for 16 h. The reaction mixture was cooled to room temperature, filtered through a celite bed, and washed with ethyl acetate (100 mL). The filtrate was concentrated to provide a crude product that was purified by flash column chromatography (SiO₂, 230-400 mesh, 15% ethyl acetate in petroleum ether) to provide tert-butyl (R)-4-(3-(2,6-bis(benzyloxy)pyridin-3-yl)-1-methyl-1H-indazol-7-yl)-3-methylpiperazine-1-carboxylate (0.62 g) as a pale yellow solid.

LC-MS (ESI): m/z=620.38 [M+H]⁺

### Preparation of (R)-6-(benzyloxy)-5-(1-methyl-7-(2-methylpiperazin-1-yl)-1H-indazol-3-yl)pyridin-2-ol

To a solution of tert-butyl (R)-4-(3-(2,6-bis(benzyloxy)pyridin-3-yl)-1-methyl-1H-indazol-7-yl)-3-methylpiperazine-1-carboxylate (0.62 g, 1.0 mmol) in DCM (12.4 mL) cooled to 0° C. was added TFA (6.2 mL). The reaction mixture was stirred at room temperature for 2 h. The reaction mixture was concentrated under reduced pressure, co-distilled with diethyl ether, and washed with n-pentane to obtain (R)-6-(benzyloxy)-5-(1-methyl-7-(2-methylpiperazin-1-yl)-1H-indazol-3-yl)pyridin-2-ol (0.40 g) as a pink semi-solid.

LC-MS (ESI): m/z=430.48 [M+H]⁺

### Preparation of benzyl (R)-4-((4-(3-(2-(benzyloxy)-6-hydroxypyridin-3-yl)-1-methyl-1H-indazol-7-yl)-3-methylpiperazin-1-yl)methyl)piperidine-1-carboxylate

To a stirred solution of (R)-6-(benzyloxy)-5-(1-methyl-7-(2-methylpiperazin-1-yl)-1H-indazol-3-yl)pyridin-2-ol (0.4 g, 0.93 mmol) and benzyl 4-formylpiperidine-1-carboxylate (0.18 g, 0.74 mmol) in THF (12 mL) was added acetic acid (0.4 mL) and sodium acetate (0.22 g, 2.79 mmol) at 0° C. The reaction mixture was stirred for 2 h. NaBH(OAc)₃ (0.59 g, 2.79 mmol) was added at 0° C., and the reaction mixture was stirred at room temperature for 16 h. The reaction mixture was quenched with ice-cold water (10 mL) and extracted with DCM (3×10 mL). Combined organic layers were washed with brine (10 mL), dried over anhydrous Na₂SO₄, filtered, and dried under vacuum to provide a crude product that was purified by column chromatography (SiO₂, 230-400, 10% methanol in DCM) to afford benzyl (R)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.28 g) as a pink semi-solid.

LC-MS (ESI): m/z=661.63 [M+H]⁺

### Preparation of 3-(1-methyl-7-((R)-2-methyl-4-(piperidin-4-ylmethyl)piperazin-1-yl)-1H-indazol-3-yl)piperidine-2,6-dione

To a stirred solution of benzyl (R)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.28 g, 0.42 mmol) in THF (5.6 mL) was added 20% Pd(OH)₂ on carbon (0.35 g, 2.54 mmol). The reaction mixture was stirred under hydrogen atmosphere (80 psi) for 16 h at rt. The reaction mixture was diluted with THF, filtered through a pad of celite, and washed with an excess amount of 20% THF in DCM.

The filtrate was collected and concentrated to provide 3-(1-methyl-7-((R)-2-methyl-4-(piperidin-4-ylmethyl)piperazin-1-yl)-1H-indazol-3-yl)piperidine-2,6-dione as a brown semi-solid (0.26 g) which was used in the next step without further purification.

LC-MS (ESI): m/z=439.51 [M+H]⁺

### Preparation of 3-(7-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2-methylpiperazin-1-yl)-1-methyl-1H-indazol-3-yl)piperidine-2,6-dione

To a solution of 3-(1-methyl-7-((R)-2-methyl-4-(piperidin-4-ylmethyl)piperazin-1-yl)-1H-indazol-3-yl)piperidine-2,6-dione (0.13 g, 0.29 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.042 g, 0.11 mmol) in DMSO (1.3 mL) was added N,N-diisopropylethylamine (0.42 mL, 0.11 mmol). The reaction mixture was stirred at 100° C. for 6 h.

The reaction mixture was quenched with ice-cold water (10 mL), and the solid precipitated was filtered and dried under vacuum to provide a crude product that was purified by prep-HPLC to afford 3-(7-((R)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-2-methylpiperazin-1-yl)-1-methyl-1H-indazol-3-yl)piperidine-2,6-dione (0.014 g) as a white solid.

LC-MS (ESI): m/z=870.53 [M+H]⁺

¹H NMR data: (400 MHz, DMSO-d6): δ 10.90 (s, 1H), 8.78 (s, 1H), 8.19 (s, 1H), 8.03 (s, 1H), 7.74 (d, J=10.40 Hz, 1H), 7.45 (d, J=9.20 Hz, 2H), 7.22 (d, J=6.80 Hz, 1H), 7.07 (t, J=14.80 Hz, 1H), 6.17 (s, 1H), 4.45-4.27 (m, 7H), 3.57 (s, 3H), 3.17 (s, 2H), 2.91-2.76 (m, 6H), 2.67-2.63 (m, 2H), 2.50 (s, 1H), 2.23-2.18 (m, 4H), 1.90-1.73 (m, 4H), 1.38-1.34 (m, 1H), 1.23 (s, 1H), 1.03 (d, J=11.60 Hz, 2H), 0.81-0.70 (m, 4H), 0.54-0.50 (m, 2H), 0.36-0.35 (m, 1H).

### Example 140: 3-(7-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 487a)

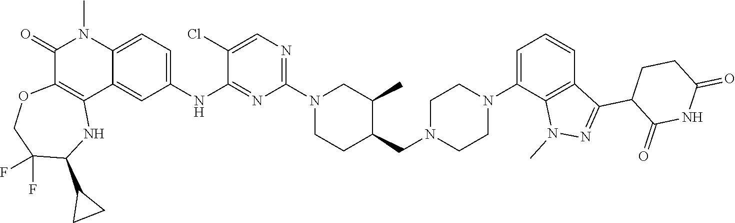 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCN(c2cccc3c(C4CCC(=O)NC4=O)nn(C)c23)CC1]

### Preparation of tert-butyl 4-(3-(2,6-bis(benzyloxy)pyridin-3-yl)-1-methyl-1H-indazol-7-yl) piperazine-1-carboxylate

A solution of 3-(2,6-bis(benzyloxy)pyridin-3-yl)-7-bromo-1-methyl-1H-indazole (1.0 g, 1.99 mmol), 1-Boc-piperazine (0.17 g, 2.0 mmol), and cesium carbonate (1.95 g, 5.99 mmol) in 1,4-dioxane (20.0 mL) was purged with argon for 10 minutes. Pd-PEPPSI-iHeptCl (0.10 g, 0.10 mmol) was then added, and the mixture was stirred and heated to 100° C. for 12 h. The reaction mixture was diluted with EtOAc (100 mL) and quenched with water (30 mL). The separated organic layer was dried over anhydrous Na₂SO₄, filtered, and concentrated under vacuum to provide a crude product that was purified by flash column chromatography (SiO₂, 230-400 mesh; 20% EtOAc in petroleum ether) to afford tert-butyl 4-(3-(2,6-bis(benzyloxy)pyridin-3-yl)-1-methyl-1H-indazol-7-yl) piperazine-1-carboxylate (0.70 g) as a pale brown semi-solid.

LCMS (ESI): m/z=506.55 [M-Boc+H]⁺

### Preparation of tert-butyl (3S, 4S)-4-(4-(3-(2,6-bis(benzyloxy)pyridin-3-yl)-1-methyl-1H-indazol-7-yl) piperazine-1-carbonyl)-3-methylpiperidine-1-carboxylate

To a solution of tert-butyl 4-(3-(2,6-bis(benzyloxy)pyridin-3-yl)-1-methyl-1H-indazol-7-yl) piperazine-1-carboxylate (0.77 g, 1.86 mmol) and (3S, 4S)-1-(tert-butoxycarbonyl)-3-methylpiperidine-4-carboxylic acid (0.45 g, 1.86 mmol) in DMF (9.40 mL) was added DIPEA (2.30 mL, 13.01 mmol) followed by HATU (1.06 g, 2.79 mmol) at 0° C. The resultant reaction mixture was stirred at room temperature for 1 h. The reaction mixture was poured into ice-cold water (20 mL), and the precipitated solid was filtered and dried under vacuum to provide a crude product that was triturated with n-pentane (20 mL) to afford tert-butyl (3S, 4S)-4-(4-(3-(2,6-bis(benzyloxy)pyridin-3-yl)-1-methyl-1H-indazol-7-yl) piperazine-1-carbonyl)-3-methylpiperidine-1-carboxylate (0.50 g) as an off-white solid.

LCMS (ESI): m/z=731.73 [M+H]⁺

### Preparation of tert-butyl (3S, 4S)-4-((4-(3-(2,6-bis(benzyloxy)pyridin-3-yl)-1-methyl-1H-indazol-7-yl) piperazin-1-yl) methyl)-3-methylpiperidine-1-carboxylate

To a solution of tert-butyl (3S, 4S)-4-(4-(3-(2,6-bis(benzyloxy)pyridin-3-yl)-1-methyl-1H-indazol-7-yl) piperazine-1-carbonyl)-3-methylpiperidine-1-carboxylate (1.0 g, 1.37 mmol) in THF (10.0 mL) was added BH₃·DMS (10.0 mL) at 0° C. The reaction mixture was stirred at room temperature for 18 h. The reaction mixture was treated with methanol (30 mL) and heated at 80° C. for 18 h. The reaction mixture was concentrated under vacuum to provide a crude product that was triturated with n-pentane (20 mL) to afford tert-butyl (3S, 4S)-4-((4-(3-(2,6-bis(benzyloxy)pyridin-3-yl)-1-methyl-1H-indazol-7-yl) piperazin-1-yl) methyl)-3-methylpiperidine-1-carboxylate (0.80 g) as an off white solid. LCMS (ESI): m/z=717.71 [M+H]⁺

### Preparation of tert-butyl (3S, 4S)-4-((4-(3-(2,6-dioxopiperidin-3-yl)-1-methyl-1H-indazol-7-yl) piperazin-1-yl) methyl)-3-methylpiperidine-1-carboxylate

To a solution tert-butyl (3S, 4S)-4-((4-(3-(2,6-bis(benzyloxy)pyridin-3-yl)-1-methyl-1H-indazol-7-yl) piperazin-1-yl) methyl)-3-methylpiperidine-1-carboxylate (0.54 g, 0.75 mmol) in THF (100.0 mL) was added 20% Pd(OH)₂ on carbon (2.0 g, 400% w/w). The reaction mixture was stirred under hydrogen atmosphere (80 psi) at room temperature for 18 h. The reaction mixture was diluted with DCM (100 mL), filtered through a pad of celite, and washed with excess of 30% THF:DCM (500 mL). The collected filtrate was concentrated under vacuum to provide a crude product that was triturated with n-pentane (100 mL) to afford tert-butyl (3S, 4S)-4-((4-(3-(2,6-dioxopiperidin-3-yl)-1-methyl-1H-indazol-7-yl) piperazin-1-yl) methyl)-3-methylpiperidine-1-carboxylate (0.35 g) as an off white solid.

LCMS (ESI): m/z=539.44 [M+H]⁺

### Preparation of 3-(1-methyl-7-(4-(((3S, 4S)-3-methylpiperidin-4-yl) methyl) piperazin-1-yl)-1H-indazol-3-yl) piperidine-2,6-dione

To a solution tert-butyl (3S, 4S)-4-((4-(3-(2,6-dioxopiperidin-3-yl)-1-methyl-1H-indazol-7-yl) piperazin-1-yl) methyl)-3-methylpiperidine-1-carboxylate (0.30 g, 0.55 mmol) in DCM (6.0 mL), was added TFA (3.0 mL) at 0° C. The reaction mixture was stirred at room temperature for 2 h.

The reaction mixture was concentrated under vacuum to provide a crude product that was triturated with n-pentane (10 mL) to afford 3-(1-methyl-7-(4-(((3S, 4S)-3-methylpiperidin-4-yl) methyl) piperazin-1-yl)-1H-indazol-3-yl) piperidine-2,6-dione (0.40 g) as a pale brown solid.

LCMS (ESI): m/z=439.51 [M+H]⁺

### Preparation of 3-(7-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-7-(4-(((3S, 4S)-3-methylpiperidin-4-yl) methyl) piperazin-1-yl)-1H-indazol-3-yl) piperidine-2,6-dione (0.17 g, 0.39 mmol) and DIPEA (1.70 mL) in DMSO (3.40 mL) was added (S)-2-cyclopropyl-10-((2,5-dichloropyrimidin-4-yl)amino)-3,3-difluoro-7-methyl-1,2,3,4-tetrahydro-[1,4]oxazepino [2,3-c]quinolin-6(7H)-one (0.04 g, 0.08 mmol). The reaction mixture was heated at 100° C. for 18 h. The reaction mixture was poured into ice-cold water (20 mL), and the precipitated solid was filtered and dried under vacuum to provide a crude product that was purified by prep HPLC to afford 3-(7-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (0.05 g) as an off white solid.

LCMS (ESI): m/z=870.52 [M+H]⁺

¹H NMR (400 MHz, DMSO-d6): δ 10.88 (s, 1H), 8.72 (s, 1H), 8.14 (s, 1H), 8.01 (s, 1H), 7.77 (dd, J=1.60, 9.00 Hz, 1H), 7.46 (d, J=9.20 Hz, 1H), 7.39 (dd, J=2.40, 6.80 Hz, 1H), 7.02-7.00 (m, 2H), 6.18 (br s, 1H), 4.33-4.32 (m, 5H), 4.23 (s, 3H), 3.57 (s, 3H), 3.03-3.00 (m, 4H), 2.87-2.84 (m, 4H), 2.61-2.59 (m, 4H), 2.29-2.27 (m, 2H), 2.18-2.17 (m, 4H), 1.92 (d, J=8.80 Hz, 2H), 1.41-1.36 (m, 4H), 0.74 (d, J=9.60 Hz, 2H), 0.52 (d, J=5.60 Hz, 2H), 0.36-0.34 (m, 1H).

### Example 141: 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazin-1-yl)methyl)piperidin-1-yl)-1-methyl-1H-indazol-3-yl)piperidine-2,6-dione (Compound 485a)

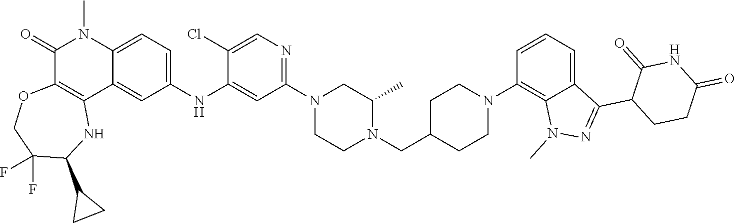 [SMILES: C[C@H]1CN(c2cc(Nc3ccc4c(c3)c3c(c(=O)n4C)OCC(F)(F)[C@H](C4CC4)N3)c(Cl)cn2)CCN1CC1CCN(c2cccc3c(C4CCC(=O)NC4=O)nn(C)c23)CC1]

### Preparation of (1-(3-(2,6-bis(benzyloxy)pyridin-3-yl)-1-methyl-1H-indazol-7-yl)piperidin-4-yl)methanol

To a suspension of 3-(2,6-bis(benzyloxy)pyridin-3-yl)-7-bromo-1-methyl-1H-indazole (2.0 g, 4.0 mmol) in 1,4-dioxane (40 mL) were added 2-(piperidin-4-yl)ethan-1-ol (1.80 g, 15.9 mmol) and cesium carbonate (3.91 g, 12.0 mmol) under nitrogen. Then, Pd-PEPPSI-iHeptCl (0.19 g, 0.20 mmol) was added, and the reaction mixture was stirred at 100° C. for 16 h. After completion, the reaction mixture was quenched with ice-cold water (30 mL) and extracted with ethyl acetate (3×150 mL). The combined organic layers were washed with brine (30 mL), dried over anhydrous Na₂SO₄, filtered, and dried under vacuum to provide a crude product that was purified by column chromatography (SiO₂, 100-200 mesh, 55% EtOAc in petroleum ether) to afford (1-(3-(2,6-bis(benzyloxy)pyridin-3-yl)-1-methyl-1H-indazol-7-yl)piperidin-4-yl)methanol (0.80 g) as a yellow solid.

LC-MS (ESI): m/z=535.57 [M+H]⁺

### Preparation of 3-(7-(4-(hydroxymethyl)piperidin-1-yl)-1-methyl-1H-indazol-3-yl)piperidine-2,6-dione

To a solution of (1-(3-(2,6-bis(benzyloxy)pyridin-3-yl)-1-methyl-1H-indazol-7-yl)piperidin-4-yl)methanol (0.8 g, 1.49 mmol) in THF (16 mL) was added 20% Pd(OH)₂ on carbon (0.80 g, 100% w/w) under argon. The mixture was stirred under hydrogen atmosphere 50 (Psi) at rt for 16 h. The reaction mixture was filtered through a celite bed and concentrated under reduced pressure to provide 3-(7-(4-(hydroxymethyl)piperidin-1-yl)-1-methyl-1H-indazol-3-yl)piperidine-2,6-dione (0.50 g) which was used in the next step without further purification.

LC-MS (ESI): m/z=357.19 [M+H]⁺

### Preparation of 1-(3-(2,6-dioxopiperidin-3-yl)-1-methyl-1H-indazol-7-yl)piperidine-4-carbaldehyde

To a solution of 3-(7-(4-(hydroxymethyl)piperidin-1-yl)-1-methyl-1H-indazol-3-yl)piperidine-2,6-dione (0.25 g, 0.70 mmol) in DCM (10 mL) was added Dess-Martin periodinane (0.6 g, 1.40 mmol) at 0° C. The reaction mixture was stirred at rt for 2 h. The reaction mixture was quenched with a saturated solution of sodium bicarbonate (20 mL) and extracted with dichloromethane (100 mL). The organic layer was separated, washed with brine (50 mL), dried over anhydrous sodium sulfate, filtered, and concentrated to obtain 1-(3-(2,6-dioxopiperidin-3-yl)-1-methyl-1H-indazol-7-yl)piperidine-4-carbaldehyde (0.25 g) which was used in the next step without further purification.

LC-MS (ESI): m/z=355.38 [M+H]⁺

### Preparation of 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazin-1-yl)methyl)piperidin-1-yl)-1-methyl-1H-indazol-3-yl)piperidine-2,6-dione

To a solution of (S)-10-((5-chloro-2-((S)-3-methylpiperazin-1-yl)pyridin-4-yl)amino)-2-cyclopropyl-3,3-difluoro-7-methyl-1,2,3,4-tetrahydro-[1,4]oxazepino[2,3-c]quinolin-6(7H)-one (0.25 g, 0.47 mmol) and 1-(3-(2,6-dioxopiperidin-3-yl)-1-methyl-1H-indazol-7-yl)piperidine-4-carbaldehyde (0.2 g, 0.56 mmol) in THF (5 mL) was added AcOH (0.3 mL) at room temperature.

The reaction mixture was stirred for 4 h. Sodium triacetoxyborohydride (0.3 g, 1.41 mmol) was added at 0° C., and the resulting mixture was stirred at room temperature for 16 h. The reaction was concentrated under reduced pressure to provide a residue that was purified by prep-HPLC to afford 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-methylpiperazin-1-yl)methyl)piperidin-1-yl)-1-methyl-1H-indazol-3-yl)piperidine-2,6-dione (0.15 g) as an off white solid.

LC-MS (ESI): m/z=869.47 [M+H]⁺

¹H NMR (400 MHz, DMSO-d6): δ 10.90 (s, 1H), 8.22 (s, 1H), 8.05 (d, J=4.4 Hz, 2H), 7.92 (s, 1H), 7.55-745 (m, 2H), 7.36 (dd, J=4.0, 2.4 Hz, 1H), 7.05-6.95 (m, 2H), 6.41 (br s, 1H), 6.08 (s, 1H), 4.55-4.30 (m, 3H), 4.22 (s, 3H), 3.67 (d, J=12 Hz, 1H), 3.60-3.50 (m, 4H), 3.20-3.00 (m, 3H), 2.95-2.70 (m, 2H), 2.75-2.60 (m, 1H), 2.60-2.55 (m, 4H), 2.44-2.35 (m, 1H), 2.30-2.20 (m, 1H), 2.20-2.10 (m, 2H), 2.10-1.95 (m, 2H), 1.80-1.75 (s, 1H), 1.75-1.60 (s, 1H) 1.55-1.30 (m, 3H), 0.99 (d, J=8 Hz, 3H), 0.7 (br s, 1H), 0.5 (br s, 2H), 0.31 (br s, 1H).

### Example 142: 3-(7-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 487b)

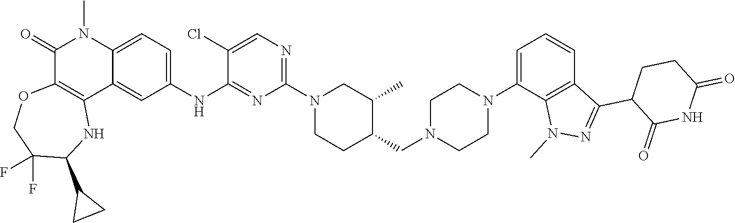 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCN(c2cccc3c(C4CCC(=O)NC4=O)nn(C)c23)CC1]

### Preparation of tert-butyl (3R, 4R)-4-(4-(3-(2,6-bis(benzyloxy)pyridin-3-yl)-1-methyl-1H-indazol-7-yl)piperazine-1-carbonyl)-3-methylpiperidine-1-carboxylate

To a solution of 3-(2,6-bis(benzyloxy)pyridin-3-yl)-1-methyl-7-(piperazin-1-yl)-1H-indazole (1.00 g, 1.98 mmol) and (3R, 4R)-1-(tert-butoxycarbonyl)-3-methylpiperidine-4-carboxylic acid (0.48 g, 1.98 mmol) in DMF (10 mL) was added DIPEA (1.70 mL, 9.89 mmol) followed by HATU (1.13 g, 2.97 mmol) at 0° C. The resultant reaction mixture was stirred at room temperature for 1 h. The reaction mixture was quenched with ice cold water (100 mL), and the precipitated solid was filtered and dried under vacuum to afford tert-butyl (3R, 4R)-4-(4-(3-(2,6-bis(benzyloxy)pyridin-3-yl)-1-methyl-1H-indazol-7-yl)piperazine-1-carbonyl)-3-methylpiperidine-1-carboxylate (1.21 g) as a pale brown solid which was used in the next step without further purification.

LC-MS (ESI): m/z=731.50 [M+H]⁺.

### Preparation of tert-butyl (3R, 4R)-4-((4-(3-(2,6-bis(benzyloxy)pyridin-3-yl)-1-methyl-1H-indazol-7-yl)piperazin-1-yl)methyl)-3-methylpiperidine-1-carboxylate

To a solution of tert-butyl (3R, 4R)-4-(4-(3-(2,6-bis(benzyloxy)pyridin-3-yl)-1-methyl-1H-indazol-7-yl)piperazine-1-carbonyl)-3-methylpiperidine-1-carboxylate (1.21 g, 1.65 mmol) in THF (24.20 mL) was added borane dimethyl sulfide (neat, 12.10 mL) at 0° C. The resultant reaction mixture was stirred at room temperature for 18 h. The reaction mixture was quenched with methanol and concentrated to provide a crude product. A stirred solution of the crude product in methanol (12 mL) was heated at 100° C. for 3 h. The reaction mixture was concentrated to provide a residue that was purified by flash chromatography using 230-400 mesh silica gel and 5-10% of ethyl acetate in petroleum ether as eluent to afford tert-butyl (3R, 4R)-4-((4-(3-(2,6-bis(benzyloxy)pyridin-3-yl)-1-methyl-1H-indazol-7-yl)piperazin-1-yl)methyl)-3-methylpiperidine-1-carboxylate as a pale yellow semi-solid (0.91 g).

LC-MS (ESI): m/z=717.75 [M+H]⁺.

### Preparation of tert-butyl (3R, 4R)-4-((4-(3-(2,6-dioxopiperidin-3-yl)-1-methyl-1H-indazol-7-yl)piperazin-1-yl)methyl)-3-methylpiperidine-1-carboxylate

To a solution of tert-butyl (3R, 4R)-4-((4-(3-(2,6-bis(benzyloxy)pyridin-3-yl)-1-methyl-1H-indazol-7-yl)piperazin-1-yl)methyl)-3-methylpiperidine-1-carboxylate (0.90 g, 1.25 mmol) in THF (100 mL) was added 20% Pd(OH)₂ on carbon (1.35 g, 150% w/w). The reaction mixture was stirred under hydrogen atmosphere (80 psi) at room temperature for 18 h. The reaction mixture was diluted with DCM (200 mL), washed with 30% THF:DCM (300 mL), and filtered through a celite bed. The filtrate was collected and concentrated under vacuum to provide a crude product that was triturated with n-pentane to afford tert-butyl (3R, 4R)-4-((4-(3-(2,6-dioxopiperidin-3-yl)-1-methyl-1H-indazol-7-yl)piperazin-1-yl)methyl)-3-methylpiperidine-1-carboxylate (0.72 g) as an off white solid.

LC-MS (ESI): m/z=539.41 [M+H]⁺.

### Preparation of 3-(1-methyl-7-(4-(((3R, 4R)-3-methylpiperidin-4-yl)methyl)piperazin-1-yl)-1H-indazol-3-yl)piperidine-2,6-dione

To a solution of tert-butyl (3R, 4R)-4-((4-(3-(2,6-dioxopiperidin-3-yl)-1-methyl-1H-indazol-7-yl)piperazin-1-yl)methyl)-3-methylpiperidine-1-carboxylate (0.72 g, 1.34 mmol) in DCM (7 mL) was added TFA (5.76 mL) at 0° C. and stirred at room temperature for 2 h. The reaction mixture was concentrated under reduced pressure to afford 3-(1-methyl-7-(4-(((3R, 4R)-3-methylpiperidin-4-yl)methyl)piperazin-1-yl)-1H-indazol-3-yl)piperidine-2,6-dione (0.74 g) as a pale brown semi solid which was used in the next step without purification.

LC-MS (ESI): 439.28 [M+H]⁺

### Preparation of 3-(7-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-7-(4-(((3R, 4R)-3-methylpiperidin-4-yl)methyl)piperazin-1-yl)-1H-indazol-3-yl)piperidine-2,6-dione (0.37 g, 0.84 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.16 g, 0.34 mmol) in DMSO (3.70 mL) was added DIPEA (3.70 mL). The resulting reaction mixture was stirred and heated at 100° C. for 18 h. The reaction mixture was quenched with ice cold water (100 mL), and the precipitated solid was filtered and dried under vacuum to provide a crude product that was purified by prep-HPLC to afford 3-(7-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (67 mg) as an off-white solid.

LC-MS (ESI): m/z=870.48 [M+H]⁺.

¹H NMR (400 MHz, DMSO-d₆): δ 10.8 (s, 1H), 8.74 (s, 1H), 8.35 (s, 1H), 8.15 (s, 1H), 8.00 (s, 1H), 7.76 (d, J=7.40 Hz, 1H), 7.45 (d, J=9.20 Hz, 1H), 7.40-7.38 (m, 1H), 7.03-7.00 (m, 2H), 6.17 (brs, 1H), 4.41-4.32 (m, 5H), 4.23 (s, 3H), 3.57 (s, 3H), 3.01-2.84 (m, 8H), 2.67-2.59 (m, 3H), 2.32-2.20 (m, 6H), 2.17-1.90 (m, 2H), 1.35 (m, 4H), 0.77-0.75 (m, 2H), 0.54 (m, 2H) 0.52 (m, 1H).

### Example 143: 3-(7-(4-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 487c)

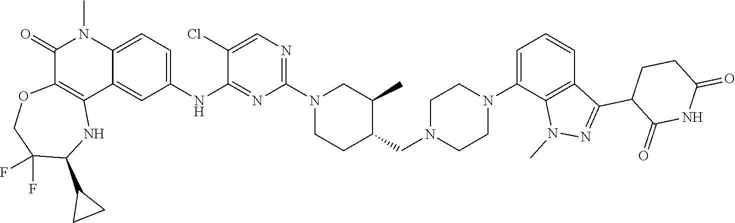 [SMILES: C[C@@H]1CN(c2ncc(Cl)c(Nc3ccc4c(c3)c3c(c(=O)n4C)OCC(F)(F)[C@H](C4CC4)N3)n2)CC[C@H]1CN1CCN(c2cccc3c(C4CCC(=O)NC4=O)nn(C)c23)CC1]

### Preparation of tert-butyl (3S, 4R)-4-(4-(3-(2,6-bis(benzyloxy)pyridin-3-yl)-1-methyl-1H-indazol-7-yl)piperazine-1-carbonyl)-3-methylpiperidine-1-carboxylate

To a solution of (3S, 4R)-1-(tert-butoxycarbonyl)-3-methylpiperidine-4-carboxylic acid (0.50 g, 2.05 mmol) and 3-(2,6-bis(benzyloxy)pyridin-3-yl)-1-methyl-7-(piperazin-1-yl)-1H-indazole (1.0 g, 2.05 mmol) in DMF (10 mL) was added DIPEA (1.41 mL, 8.22 mmol). The reaction mixture was stirred for 10 min, and HATU (1.17 g, 3.08 mmol) was added at 0° C. The resulting mixture was stirred at rt for 2 h. After completion, the reaction mixture was diluted with ethyl acetate (30 mL) and washed with cold water (3×20 mL). The organic layer was dried over anhydrous sodium sulfate and concentrated under reduced pressure to provide a crude product that was triturated with n-pentane to afford tert-butyl (3S, 4R)-4-(4-(3-(2,6-bis(benzyloxy)pyridin-3-yl)-1-methyl-1H-indazol-7-yl)piperazine-1-carbonyl)-3-methylpiperidine-1-carboxylate (1.1 g) as an off-white solid.

LC-MS (ESI): m/z=731.77 [M+H]⁺

### Preparation of tert-butyl (3S, 4R)-4-((4-(3-(2,6-bis(benzyloxy)pyridin-3-yl)-1-methyl-1H-indazol-7-yl)piperazin-1-yl)methyl)-3-methylpiperidine-1-carboxylate

To a solution of tert-butyl (3S, 4R)-4-(4-(3-(2,6-bis(benzyloxy)pyridin-3-yl)-1-methyl-1H-indazol-7-yl)piperazine-1-carbonyl)-3-methylpiperidine-1-carboxylate (1.1 g, 1.50 mmol) in THF (22 mL) was added borane dimethylsulfide solution in THF (2.0 M, 11.0 mL). The reaction mixture was stirred at rt for 8 h. After completion, the reaction mixture was treated with methanol and evaporated under reduced pressure. The resulting residue was dissolved in methanol and refluxed at 80° C. for 2 h. The reaction mixture was concentrated under reduced pressure to provide a crude product that was purified by flash column chromatography (SiO₂, 100-200 mesh, 50% ethyl acetate in petroleum ether) to afford tert-butyl (3S, 4R)-4-((4-(3-(2,6-bis(benzyloxy)pyridin-3-yl)-1-methyl-1H-indazol-7-yl)piperazin-1-yl)methyl)-3-methylpiperidine-1-carboxylate (0.8 g) as a brown semi-solid.

LC-MS (ESI): m/z=717.79 [M+H]⁺.

### Preparation of tert-butyl (3S, 4R)-4-((4-(3-(2,6-dioxopiperidin-3-yl)-1-methyl-1H-indazol-7-yl)piperazin-1-yl)methyl)-3-methylpiperidine-1-carboxylate

To a solution of tert-butyl (3S, 4R)-4-((4-(3-(2,6-bis(benzyloxy)pyridin-3-yl)-1-methyl-1H-indazol-7-yl)piperazin-1-yl)methyl)-3-methylpiperidine-1-carboxylate (0.80 g, 1.11 mmol) in THF (24.0 mL) was added 20% Pd(OH)₂ on carbon (0.8 g). The reaction mixture was stirred under hydrogen atmosphere (80 Psi) at room temperature for 16 h. The reaction mixture was diluted with ethyl acetate (50 mL), filtered through a celite bed, and washed with 50% THF in ethyl acetate (200 mL). The collected filtrate was concentrated under reduced pressure to provide a crude product that was triturated with diethyl ether to afford tert-butyl (3S, 4R)-4-((4-(3-(2,6-dioxopiperidin-3-yl)-1-methyl-1H-indazol-7-yl)piperazin-1-yl)methyl)-3-methylpiperidine-1-carboxylate (0.60 g) as a brown semi-solid.

LC-MS (ESI): m/z=539.62 [M+H]⁺.

### Preparation of 3-(1-methyl-7-(4-(((3S, 4R)-3-methylpiperidin-4-yl)methyl)piperazin-1-yl)-1H-indazol-3-yl)piperidine-2,6-dione

To a solution of tert-butyl (3S, 4R)-4-((4-(3-(2,6-dioxopiperidin-3-yl)-1-methyl-1H-indazol-7-yl)piperazin-1-yl)methyl)-3-methylpiperidine-1-carboxylate (0.60 g, 1.11 mmol) in DCM (6.0 mL) was added TFA (3.0 mL). The reaction mixture was stirred at room temperature for 1 h. The reaction mixture was concentrated and co-distilled with petroleum ether under reduced pressure to provide a crude product that was triturated with diethyl ether to afford 3-(1-methyl-7-(4-(((3S, 4R)-3-methylpiperidin-4-yl)methyl)piperazin-1-yl)-1H-indazol-3-yl)piperidine-2,6-dione (0.45 g) as an off white solid.

LC-MS (ESI): m/z=439.55 [M+H]⁺.

### Preparation of 3-(7-(4-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-7-(4-(((3S, 4R)-3-methylpiperidin-4-yl)methyl)piperazin-1-yl)-1H-indazol-3-yl)piperidine-2,6-dione (0.25 g, 0.57 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.17 mmol) in DMSO (5 mL) was added N,N-diisopropylethylamine (0.5 mL, 2.85 mmol). The resulting reaction mixture was stirred at 100° C. for 6 h. The reaction mixture was poured into ice-cold water and stirred for 15 minutes. The resulting precipitate was filtered and dried to provide a crude product that was purified by prep-HPLC to afford 3-(7-(4-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (58 mg) as a white solid.

LC-MS (ESI): m/z=870.50 [M+H]⁺.

¹H NMR (400 MHz, DMSO-d₆): δ 10.87 (s, 1H), 8.79 (s, 1H), 8.39 (s, 1H), 8.16 (s, 1H), 8.03 (s, 1H), 7.74 (d, J=9.20 Hz, 1H), 7.44 (d, J=9.20 Hz, 1H), 7.39 (t, J=4.80 Hz, 1H), 7.03 (d, J=4.40 Hz, 2H), 6.15 (s, 1H), 4.33-4.31 (m, 5H), 4.23 (s, 3H), 3.57 (s, 3H), 2.80-2.77 (m, 6H), 2.62-2.61 (m, 2H), 2.46-2.44 (br, 4H), 2.13-2.10 (m, 3H), 1.93-1.90 (m, 1H), 1.50-1.24 (m, 4H), 1.11-0.98 (m, 1H), 0.90 (d, J=6.80 Hz, 3H), 0.79-0.61 (m, 1H), 0.52-0.50 (m, 2H), 0.40-0.35 (m, 1H).

### Example 144: 3-(7-(4-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (Compound 487d)

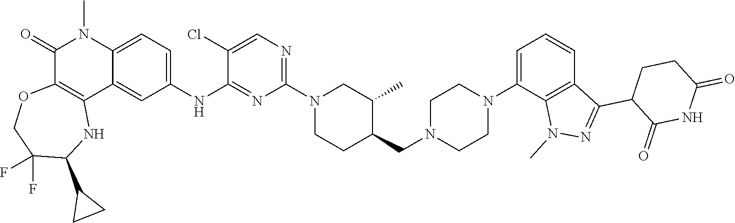 [SMILES: C[C@H]1CN(c2ncc(Cl)c(Nc3ccc4c(c3)c3c(c(=O)n4C)OCC(F)(F)[C@H](C4CC4)N3)n2)CC[C@@H]1CN1CCN(c2cccc3c(C4CCC(=O)NC4=O)nn(C)c23)CC1]

### Preparation of 3-(2,6-bis(benzyloxy)pyridin-3-yl)-1-methyl-7-(piperazin-1-yl)-1H-indazole

To a solution of 3-(2,6-bis(benzyloxy)pyridin-3-yl)-7-bromo-1-methyl-1H-indazole (5.0 g, 9.99 mmol) and piperazine (5.0 g, 19.98 mmol) in 1,4-dioxane (100 mL) was added cesium carbonate (9.76 g, 29.97 mmol). The reaction mixture was degassed with argon for 15 minutes.

Then Pd-PEPPSI-IHeptCl (0.48 g, 0.49 mmol) was added, and the resulting reaction mixture was stirred at 100° C. for 16 h. The reaction mixture was diluted with EtOAc (50 mL), filtered through a celite bed, and washed with ethyl acetate (100 mL). The collected filtrate was concentrated under reduced pressure to provide a crude product that was purified by flash column chromatography (SiO₂, 100-200 mesh, 50% ethyl acetate in petroleum ether) to afford 3-(2,6-bis(benzyloxy)pyridin-3-yl)-1-methyl-7-(piperazin-1-yl)-1H-indazole (3.0 g) as a brown semi-solid.

LC-MS (ESI): m/z=506.65 [M+H]⁺.

### Preparation of tert-butyl (3R, 4S)-4-(4-(3-(2,6-bis(benzyloxy)pyridin-3-yl)-1-methyl-1H-indazol-7-yl)piperazine-1-carbonyl)-3-methylpiperidine-1-carboxylate

To a solution of (3R, 4S)-1-(tert-butoxycarbonyl)-3-methylpiperidine-4-carboxylic acid (0.48 g, 1.97 mmol) and 3-(2,6-bis(benzyloxy)pyridin-3-yl)-1-methyl-7-(piperazin-1-yl)-1H-indazole (1.0 g, 1.97 mmol) in THF (20 mL) was added DIPEA (1.37 mL, 7.88 mmol). The resulting mixture was stirred for 10 min. HATU (108 mg, 0.28 mmol) was then added at 0° C., and the reaction mixture was stirred at room temperature for 2 h. After completion, the reaction mixture was diluted with ethyl acetate (30 mL) and washed with cold water (3×20 mL). The organic layer was dried over anhydrous sodium sulfate and concentrated under reduced pressure. The resulting residue was triturated with n-pentane to afford tert-butyl (3R, 4S)-4-(4-(3-(2,6-bis(benzyloxy)pyridin-3-yl)-1-methyl-1H-indazol-7-yl)piperazine-1-carbonyl)-3-methylpiperidine-1-carboxylate (1.3 g) as an off white solid.

LC-MS (ESI): m/z=731.77 [M+H]⁺.

### Preparation of tert-butyl (3R, 4S)-4-((4-(3-(2,6-bis(benzyloxy)pyridin-3-yl)-1-methyl-1H-indazol-7-yl)piperazin-1-yl)methyl)-3-methylpiperidine-1-carboxylate

To a solution of tert-butyl (3R, 4S)-4-(4-(3-(2,6-bis(benzyloxy)pyridin-3-yl)-1-methyl-1H-indazol-7-yl)piperazine-1-carbonyl)-3-methylpiperidine-1-carboxylate (1.3 g, 1.77 mmol) in THF (26 mL) was added borane dimethylsulfide solution in THF (2.0 M, 26 mL). The reaction mixture was stirred at rt for 8 h. After completion, the reaction mixture was treated with methanol and evaporated under reduced pressure. Then the resulting residue was dissolved in methanol and refluxed at 80° C. for 2 h. The reaction mixture was concentrated under reduced pressure to provide a crude product that was purified by flash column chromatography (SiO₂, 100-200 mesh, 50% ethyl acetate in petroleum ether) to afford tert-butyl (3R, 4S)-4-((4-(3-(2,6-bis(benzyloxy)pyridin-3-yl)-1-methyl-1H-indazol-7-yl)piperazin-1-yl)methyl)-3-methylpiperidine-1-carboxylate (0.9 g) as a brown semi-solid.

LC-MS (ESI): m/z=717.75 [M+H]⁺.

### Preparation of tert-butyl (3R, 4S)-4-((4-(3-(2,6-dioxopiperidin-3-yl)-1-methyl-1H-indazol-7-yl)piperazin-1-yl)methyl)-3-methylpiperidine-1-carboxylate

To a solution of tert-butyl (3R, 4S)-4-((4-(3-(2,6-bis(benzyloxy)pyridin-3-yl)-1-methyl-1H-indazol-7-yl)piperazin-1-yl)methyl)-3-methylpiperidine-1-carboxylate (0.90 g, 1.25 mmol) in THF (45.0 mL) was added 20% Pd (OH)₂ on carbon, moisture 50% wet (3.6 g). The reaction mixture was stirred under hydrogen atmosphere (80 Psi) at room temperature for 16 h. The reaction mixture was diluted with ethyl acetate (50 mL), filtered through a celite bed, and washed with 50% THF in ethyl acetate (200 mL). The collected filtrate was concentrated under reduced pressure to provide a crude product that was triturated with diethyl ether to afford tert-butyl (3R, 4S)-4-((4-(3-(2,6-dioxopiperidin-3-yl)-1-methyl-1H-indazol-7-yl)piperazin-1-yl)methyl)-3-methylpiperidine-1-carboxylate (0.60 g) as a brown semi-solid.

LC-MS (ESI): m/z=539.67 [M+H]⁺.

### Preparation of 3-(1-methyl-7-(4-(((3R, 4S)-3-methylpiperidin-4-yl)methyl)piperazin-1-yl)-1H-indazol-3-yl)piperidine-2,6-dione

To a solution of tert-butyl (3R, 4S)-4-((4-(3-(2,6-dioxopiperidin-3-yl)-1-methyl-1H-indazol-7-yl)piperazin-1-yl)methyl)-3-methylpiperidine-1-carboxylate (0.60 g, 1.11 mmol) in DCM (6.0 mL) was added TFA (6.0 mL). The reaction mixture was stirred at room temperature for 1 h. The reaction mixture was concentrated and co-distilled with petroleum ether under reduced pressure to provide a crude product that was triturated with diethyl ether to afford 3-(1-methyl-7-(4-(((3R, 4S)-3-methylpiperidin-4-yl)methyl)piperazin-1-yl)-1H-indazol-3-yl)piperidine-2,6-dione (0.4 g) as an off white solid.

LC-MS (ESI): m/z=439.55 [M+H]⁺.

### Preparation of 3-(7-(4-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione

To a solution of 3-(1-methyl-7-(4-(((3R, 4S)-3-methylpiperidin-4-yl)methyl)piperazin-1-yl)-1H-indazol-3-yl)piperidine-2,6-dione (0.2 g, 0.45 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.13 mmol) in DMSO (4 mL) was added N,N-diisopropylethylamine (0.39 g, 2.28 mmol). The reaction mixture was stirred at 100° C. for 6 h.

The reaction mixture was poured into ice-cold water and stirred for 15 minutes. The resulting precipitate was filtered and dried to provide a crude product that was purified by prep-HPLC to afford 3-(7-(4-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-1-methyl-1H-indazol-3-yl)piperidine-2,6-dione (0.06 g) as a white solid.

LC-MS (ESI): m/z=870.46 [M+H]⁺.

¹H NMR (400 MHz, DMSO-d6): δ 10.88 (s, 1H), 8.78 (s, 1H), 8.17 (d, J=1.60 Hz, 1H), 8.02 (s, 1H), 7.75 (d, J=8.00 Hz, 1H), 7.45 (d, J=9.20 Hz, 1H), 7.38-7.38 (m, 1H), 7.02-7.00 (m, 2H), 6.21 (br s, 1H), 4.33-4.31 (m, 5H), 4.24 (s, 3H), 3.57 (s, 3H), 2.79-2.76 (m, 8H), 2.59-2.58 (m, 3H), 2.45-2.44 (m, 2H), 2.32-2.30 (m, 1H), 2.17-2.16 (m, 3H), 2.11-1.90 (m, 1H), 1.50-1.30 (m, 3H), 1.09-1.05 (m, 1H), 0.89-0.87 (m, 3H), 0.72-0.70 (m, 1H), 0.51-0.50 (m, 2H), 0.36-0.34 (m, 1H).

### Example 145. 3-(7-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazine-1-carbonyl)piperidin-1-yl)-1-methyl-1H-indazol-3-yl)piperidine-2,6-dione (Compound 432a)

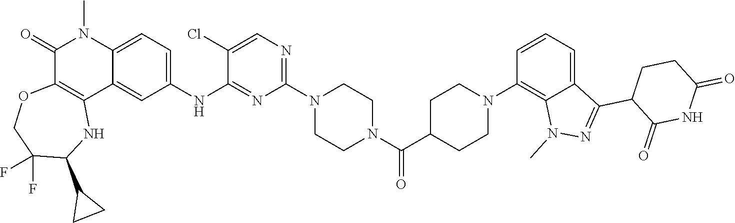 [SMILES: Cn1nc(C2CCC(=O)NC2=O)c2cccc(N3CCC(C(=O)N4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 1-(3-(2,6-bis(benzyloxy)pyridin-3-yl)-1-methyl-1H-indazol-7-yl)piperidine-4-carboxylate

In a vial, 3-(2,6-bis(benzyloxy)pyridin-3-yl)-7-bromo-1-methyl-1H-indazole (600 mg, 1.20 mmol), 4-piperidinecarboxylic acid t-butyl ester HCl (306 mg, 1.38 mmol), 1,4-dioxane (10 mL), and chloro(2-dicyclohexylphosphino-2′,6′-di-1-propoxy-1,1′-biphenyl)(2′-amino-1,1′-biphenyl-2-yl)palladium(II) (74.5 mg, 95.9 μmol) were combined. The reaction mixture was purged with nitrogen for 5 minutes. Sodium tert-butoxide (461 mg, 2.40 mL, 2.0 M, 4.80 mmol) was then added, and the reaction was stirred and heated at 100° C. After heating for 5 hours, the reaction was quenched with water. The reaction was diluted with DCM and the layers separated. The organic layer was washed with saturated NaCl, dried with MgSO₄, filtered, and purified via flash column chromatography (0-100% ethyl acetate in heptanes) to afford tert-butyl 1-(3-(2,6-bis(benzyloxy)pyridin-3-yl)-1-methyl-1H-indazol-7-yl)piperidine-4-carboxylate (474.7 mg).

¹H NMR (400 MHz, DMSO) δ 7.95-7.82 (m, 1H), 7.54-7.23 (m, 12H), 7.04-6.89 (m, 2H), 6.59 (d, J=8.0 Hz, 1H), 5.43 (d, J=5.3 Hz, 4H), 4.32 (s, 3H), 3.27 (d, J=11.4 Hz, 2H), 2.72 (t, J=12.1 Hz, 2H), 2.35 (d, J=12.0 Hz, 1H), 2.02-1.93 (m, 3H), 1.78 (d, J=12.7 Hz, 2H), 1.44 (s, 9H).

### Preparation of tert-butyl 1-(3-(2,6-dioxopiperidin-3-yl)-1-methyl-1H-indazol-7-yl)piperidine-4-carboxylate

In a 250 m1 round bottom flask, tert-butyl 1-(3-(2,6-bis(benzyloxy)pyridin-3-yl)-1-methyl-1H-indazol-7-yl)piperidine-4-carboxylate (474.7 mg, 706.5 μmol) was dissolved in EtOAc (120 mL).

The reaction was flushed with nitrogen for 5 minutes, and then palladium on carbon (75.18 mg) was added. The reaction was put under hydrogen via balloon and heated to 60° C. for 18 hours.

The reaction was filtered through celite and concentrated to afford tert-butyl 1-(3-(2,6-dioxopiperidin-3-yl)-1-methyl-1H-indazol-7-yl)piperidine-4-carboxylate (308 mg).

¹H NMR (400 MHz, DMSO) δ 10.88 (s, 1H), 7.43-7.34 (m, 1H), 7.06-6.97 (m, 2H), 4.36 (ddd, J=19.9, 9.6, 5.1 Hz, 1H), 4.24 (s, 3H), 3.25 (s, 2H), 2.74-2.55 (m, 2H), 2.34 (ddt, J=13.8, 9.3, 4.6 Hz, 1H), 2.24-2.07 (m, 1H), 1.94 (d, J=15.6 Hz, 2H), 1.77 (d, J=12.7 Hz, 1H), 1.44 (s, 9H).

### Preparation of 1-(3-(2,6-dioxopiperidin-3-yl)-1-methyl-1H-indazol-7-yl)piperidine-4-carboxylic acid

In a vial, tert-butyl 1-(3-(2,6-dioxopiperidin-3-yl)-1-methyl-1H-indazol-7-yl)piperidine-4-carboxylate (308.4 mg, 650.8 mol) was dissolved in DCM (6 mL). Trifluoroacetic acid (0.4957 mL) was added, and the reaction was stirred at room temperature for 3 hours. The reaction was concentrated under reduced pressure and the crude product was diluted with DMSO (3 mL), filtered through a syringe filter, and purified via ACCQPrep to afford 1-(3-(2,6-dioxopiperidin-3-yl)-1-methyl-1H-indazol-7-yl)piperidine-4-carboxylic acid (174.6 mg).

LC-MS (ESI): m/z=371.5 [M+H]⁺.

### Preparation of tert-butyl 4-(1-(3-(2,6-dioxopiperidin-3-yl)-1-methyl-1H-indazol-7-yl)piperidine-4-carbonyl)piperazine-1-carboxylate

In a vial, 1-(3-(2,6-dioxopiperidin-3-yl)-1-methyl-1H-indazol-7-yl)piperidine-4-carboxylic acid (131.7 mg, 355.6 mol), tert-butyl-1-piperazinecarboxylate (72.85 mg, 391.1 mol), HATU (162.2 mg, 426.7 mol), DMF (3 mL), and diisopropylethylamine (138 mg, 1.06 mmol) were combined. The reaction was stirred at room temperature for 18 hours. The reaction was filtered via syringe filter and purified via ACCQPrep to afford tert-butyl 4-(1-(3-(2,6-dioxopiperidin-3-yl)-1-methyl-1H-indazol-7-yl)piperidine-4-carbonyl)piperazine-1-carboxylate (158.2 mg).

LC-MS (ESI): m/z=539.8 [M+H]⁺.

### Preparation of 3-(1-methyl-7-(4-(piperazine-1-carbonyl)piperidin-1-yl)-1H-indazol-3-yl)piperidine-2,6-dione

In a vial, tert-butyl 4-(1-(3-(2,6-dioxopiperidin-3-yl)-1-methyl-1H-indazol-7-yl)piperidine-4-carbonyl)piperazine-1-carboxylate (158.2 mg, 293.7 mol) was dissolved in DCM (8 mL).

Trifluoroacetic acid (334.9 mg, 2.937 mmol) was then added, and the reaction was stirred for 3 hours. The reaction was concentrated to afford 3-(1-methyl-7-(4-(piperazine-1-carbonyl)piperidin-1-yl)-1H-indazol-3-yl)piperidine-2,6-dione (128.8 mg) which was used in the next step without further purification.

LC-MS (ESI): m/z=439.4 [M+H]⁺.

### Preparation of 3-(7-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazine-1-carbonyl)piperidin-1-yl)-1-methyl-1H-indazol-3-yl)piperidine-2,6-dione

In a vial, 3-(1-methyl-7-(4-(piperazine-1-carbonyl)piperidin-1-yl)-1H-indazol-3-yl)piperidine-2,6-dione (65 mg, 0.15 mmol), (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (69 mg, 0.15 mmol), DMSO (3 mL), and diisopropylethylamine (0.19 g, 1.5 mmol) were combined. The reaction was stirred and heated at 85° C. for 18 hours. After cooling, the reaction was filtered via syringe filter. The crude product was purified via ACCQPrep to afford 3-(7-(4-(4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazine-1-carbonyl)piperidin-1-yl)-1-methyl-1H-indazol-3-yl)piperidine-2,6-dione (72.5 mg) as a white solid.

¹H NMR (400 MHz, DMSO) δ 10.80 (s, 1H), 8.84 (s, 1H), 8.15 (d, J=2.2 Hz, 1H), 8.02 (s, 1H), 7.64 (dd, J=9.0, 2.1 Hz, 1H), 7.39 (d, J=9.1 Hz, 1H), 7.31 (dd, J=7.6, 1.3 Hz, 1H), 7.00-6.89 (m, 2H), 6.20 (d, J=4.1 Hz, 1H), 4.47-4.22 (m, 3H), 4.17 (s, 3H), 3.53 (d, J=17.2 Hz, 10H), 3.17 (d, J=11.5 Hz, 3H), 2.84-2.50 (m, 2H), 2.26 (dtd, J=14.4, 9.4, 5.2 Hz, 1H), 2.09 (dq, J=13.2, 5.5 Hz, 1H), 1.88-1.63 (m, 4H), 1.30-1.23 (m, OH), 0.63 (h, J=6.1 Hz, 1H), 0.48-0.41 (m, 2H), 0.29 (t, J=5.8 Hz, 1H).

LC-MS (ESI): m/z=871.3 [M+H]⁺.

### Example 146. 3-(7-(7-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)-2-azaspiro[3.5]nonan-2-yl)-1-methyl-1H-indazol-3-yl)piperidine-2,6-dione (Compound 186a)

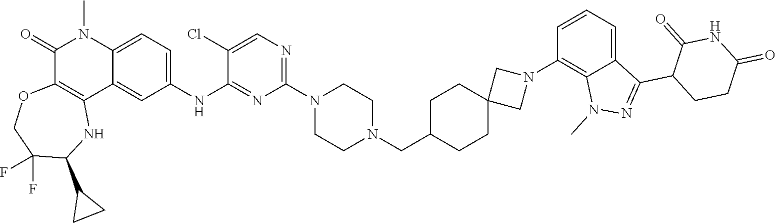 [SMILES: Cn1nc(C2CCC(=O)NC2=O)c2cccc(N3CC4(CCC(CN5CCN(c6ncc(Cl)c(Nc7ccc8c(c7)c7c(c(=O)n8C)OCC(F)(F)[C@H](C8CC8)N7)n6)CC5)CC4)C3)c21]

### Preparation of benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate

To a stirred solution of benzyl 4-((2-azaspiro[3.5]nonan-7-yl)methyl)piperazine-1-carboxylate (0.31 g, 0.84 mmol) in 1,4-dioxane (3 mL) was added 3-(2,6-bis(benzyloxy)pyridin-3-yl)-7-bromo-1-methyl-1H-indazole (0.42 g, 0.84 mmol). After degassing the reaction mixture with argon for 10 minutes, Cs₂CO₃ (0.82 g, 2.52 mmol) and Pd-PEPPSI-iHept Cl (0.041 g, 0.04 mmol) were added. The reaction was heated at 100° C. for 5 h. The reaction mixture was quenched with ice-cold water and extracted into ethyl acetate. The combined organic layers were washed with brine, dried over anhydrous Na₂SO₄, filtered, and concentrated under vacuum to afford a crude product. The crude product was purified by column chromatography (46% EtOAc in petroleum ether) to afford benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate (0.25 g) as a pale brown semi-solid.

LC-MS (ESI): m/z=777.78 [M+H]⁺.

### Preparation of 3-(1-methyl-7-(7-(piperazin-1-ylmethyl)-2-azaspiro[3.5]nonan-2-yl)-1H-indazol-3-yl)piperidine-2,6-dione

To a stirred solution of benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate (0.32 g, 0.41 mmol) in THF (9 mL), acetic acid (0.3 mL), DMF (0.3 mL), and 20% Pd(OH)₂ on carbon 50% wet basis (0.86 g, 6.15 mmol) were added. The reaction was put under hydrogen atmosphere at room temperature for 20 h. The reaction mixture was diluted with DCM, filtered through a pad of celite, and washed with an excess of 40% THF in DCM. The filtrate was concentrated under reduced pressure to afford 3-(1-methyl-7-(7-(piperazin-1-ylmethyl)-2-azaspiro[3.5]nonan-2-yl)-1H-indazol-3-yl)piperidine-2,6-dione (0.18 g) as a pale brown semi-solid.

LC-MS (ESI): m/z=465.57 [M+H]⁺.

### Preparation of 3-(7-(7-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)-2-azaspiro[3.5]nonan-2-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (46.0 mg, 98.2 mol) and 3-(1-methyl-7-(7-(piperazin-1-ylmethyl)-2-azaspiro[3.5]nonan-2-yl)-1H-indazol-3-yl)piperidine-2,6-dione hydrochloride (59.1 mg, 118 mol) in DMSO (0.700 mL) was treated with DIEA (68.4 μL, 393 mol). The reaction mixture was warmed to 80° C. for 19 h. The reaction mixture was cooled to room temperature, filtered, and purified by prep-HPLC to afford 3-(7-(7-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)-2-azaspiro[3.5]nonan-2-yl)-1-methyl-1H-indazol-3-yl)piperidine-2,6-dione (21.6 mg).

¹H NMR (400 MHz, DMSO-d₆) δ 10.87 (s, 1H), 9.59 (s, 1H), 9.05 (s, 1H), 8.20 (s, 1H), 8.13 (s, 1H), 7.75-7.61 (m, 1H), 7.44 (d, J=9.1 Hz, 1H), 7.21 (d, J=8.1 Hz, 1H), 6.98 (t, J=7.7 Hz, 1H), 6.73 (d, J=7.4 Hz, 1H), 6.24 (s, 1H), 4.59-4.36 (m, 4H), 4.31 (dd, J=9.5, 5.2 Hz, 1H), 4.17 (s, 3H), 3.70-3.59 (m, 2H), 3.57 (s, 4H), 3.52-3.42 (m, 3H), 3.28-3.14 (m, 2H), 3.04-2.89 (m, 4H), 2.72-2.57 (m, 2H), 2.36-2.24 (m, 2H), 2.19-2.12 (m, 1H), 2.06-1.93 (m, 2H), 1.82-1.69 (m, 3H), 1.52 (t, J=12.5 Hz, 2H), 1.37-1.29 (m, 1H), 1.16-1.03 (m, 1H), 0.75-0.67 (m, 1H), 0.59-0.48 (m, 2H), 0.40-0.27 (m, 1H). LC-MS (ESI): m/z=896.6 [M+H]⁺.

### Example 147. 3-(6-(4-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperazin-1-yl)-1-methyl-1H-indazol-3-yl)piperidine-2,6-dione (Compound 473a)

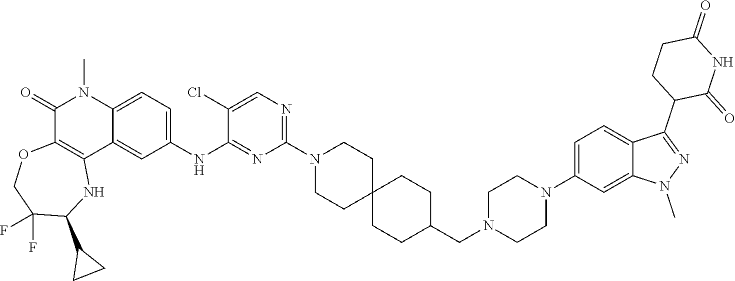 [SMILES: Cn1nc(C2CCC(=O)NC2=O)c2ccc(N3CCN(CC4CCC5(CC4)CCN(c4ncc(Cl)c(Nc6ccc7c(c6)c6c(c(=O)n7C)OCC(F)(F)[C@H](C7CC7)N6)n4)CC5)CC3)cc21]

### Preparation of tert-butyl 9-((4-(3-(2,6-dioxopiperidin-3-yl)-1-methyl-1H-indazol-6-yl)piperazin-1-yl)methyl)-3-azaspiro[5.5]undecane-3-carboxylate

A mixture of tert-butyl 9-formyl-3-azaspiro[5.5]undecane-3-carboxylate (97.4 mg, 346 mol) and 3-(1-methyl-6-(piperazin-1-yl)-1H-indazol-3-yl)piperidine-2,6-dione hydrochloride (139 mg, 381 mol) in DCM (2.00 mL) was treated with sodium triacetoxyborohydride (220 mg, 1.04 mmol) in portions over 2 minutes. The reaction mixture was then stirred at room temperature for 24 h. The reaction mixture was quenched by the addition of water (1 mL), and then the layers separated were. The aqueous phase was extracted twice with DCM. The combined organic layer was concentrated under reduced pressure, dissolved in minimal DMSO, and purified directly by reverse-phase column chromatography (10-100% MeCN—H₂O [+0.1% formic acid]) to afford tert-butyl 9-((4-(3-(2,6-dioxopiperidin-3-yl)-1-methyl-1H-indazol-6-yl)piperazin-1-yl)methyl)-3-azaspiro[5.5]undecane-3-carboxylate (17.5 mg).

LC-MS (ESI): m/z=593.4 [M+H]⁺.

### Preparation of 3-(6-(4-((3-azaspiro[5.5]undecan-9-yl)methyl)piperazin-1-yl)-1-methyl-1H-indazol-3-yl)piperidine-2,6-dione

A solution of tert-butyl 9-((4-(3-(2,6-dioxopiperidin-3-yl)-1-methyl-1H-indazol-6-yl)piperazin-1-yl)methyl)-3-azaspiro[5.5]undecane-3-carboxylate (16.0 mg, 27.0 mol) in DCM (1.00 mL) was treated with a 1:1 solution of TFA (1.00 mL) and DCM (1.00 mL). The reaction mixture stirred at room temperature for 18 h. The reaction was concentrated under reduced pressure, and the resulting residue was suspended in DCM. The mixture was concentrated again under reduced pressure to afford 3-(6-(4-((3-azaspiro[5.5]undecan-9-yl)methyl)piperazin-1-yl)-1-methyl-1H-indazol-3-yl)piperidine-2,6-dione (26.6 mg).

LC-MS (ESI): m/z=493.4 [M+H]⁺.

### Preparation of 3-(6-(4-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperazin-1-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (27.0 mg, 57.7 mol) and 3-(6-(4-((3-azaspiro[5.5]undecan-9-yl)methyl)piperazin-1-yl)-1-methyl-1H-indazol-3-yl)piperidine-2,6-dione (26.6 mg, 28.0 mol) in DMSO (1.00 mL) was treated with DIEA (34.2 μL, 196 mol). The reaction mixture was warmed to 80° C. for 16 h. The reaction mixture was cooled to room temperature, filtered, and purified by prep-HPLC to afford 3-(6-(4-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperazin-1-yl)-1-methyl-1H-indazol-3-yl)piperidine-2,6-dione (7.7 mg).

¹H NMR (400 MHz, DMSO-d₆) δ 10.83 (s, 1H), 8.75 (s, 1H), 8.19 (d, J=2.2 Hz, 1H), 8.02 (s, 1H), 7.73 (dd, J=9.1, 2.1 Hz, 1H), 7.46 (dd, J=20.3, 9.1 Hz, 2H), 6.91 (d, J=9.0 Hz, 1H), 6.83 (s, 1H), 6.17 (s, 1H), 4.56-4.30 (m, 2H), 4.25 (dd, J=9.1, 5.1 Hz, 1H), 3.88 (s, 3H), 3.64-3.50 (m, 8H), 3.31-3.25 (m, 2H), 3.25-3.14 (m, 6H), 2.64-2.57 (m, 2H), 2.31-2.24 (m, 1H), 2.20-2.08 (m, 4H), 1.73-1.61 (m, 2H), 1.60-1.48 (m, 2H), 1.47-1.39 (m, 2H), 1.35-1.17 (m, 3H), 1.15-0.98 (m, 4H), 0.81-0.62 (m, 1H), 0.51 (t, J=6.0 Hz, 2H), 0.41-0.27 (m, 1H).

LC-MS (ESI): m/z=924.6 [M+H]⁺.

### Example 148. 3-(6-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-7-azaspiro[3.5]nonan-2-yl)methyl)piperazin-1-yl)-1-methyl-1H-indazol-3-yl)piperidine-2,6-dione (Compound 477a)

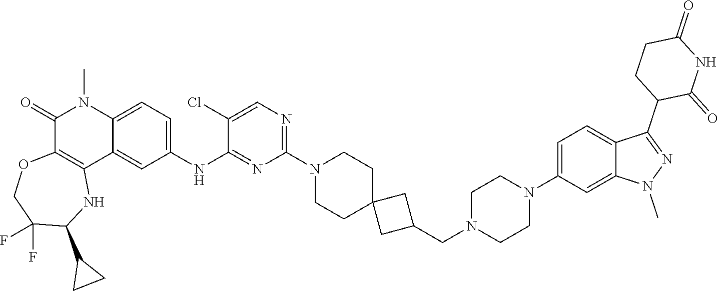 [SMILES: Cn1nc(C2CCC(=O)NC2=O)c2ccc(N3CCN(CC4CC5(CCN(c6ncc(Cl)c(Nc7ccc8c(c7)c7c(c(=O)n8C)OCC(F)(F)[C@H](C8CC8)N7)n6)CC5)C4)CC3)cc21]

### Preparation of tert-butyl 2-((4-(3-(2,6-dioxopiperidin-3-yl)-1-methyl-1H-indazol-6-yl)piperazin-1-yl)methyl)-7-azaspiro[3.5]nonane-7-carboxylate

A mixture of tert-butyl 2-formyl-7-azaspiro[3.5]nonane-7-carboxylate (91.3 mg, 360 mol) and 3-(1-methyl-6-(piperazin-1-yl)-1H-indazol-3-yl)piperidine-2,6-dione, HCl (144 mg, 396 mol) in DCM (2.00 mL) was treated with sodium triacetoxyborohydride (229 mg, 1.08 mmol) in portions over 2 min. The reaction mixture was allowed to stir at room temperature. After 24 h, the reaction mixture was quenched by the addition of water (1 mL). The mixture was stirred vigorously for 5 minutes, and then the layers were separated. The aqueous phase was extracted twice more with DCM, then the combined organic layer was concentrated under reduced pressure, dissolved in minimal DMSO, and purified directly by reverse-phase column chromatography (10-100% MeCN—H₂O [+0.1% formic acid]) to afford tert-butyl 2-((4-(3-(2,6-dioxopiperidin-3-yl)-1-methyl-1H-indazol-6-yl)piperazin-1-yl)methyl)-7-azaspiro[3.5]nonane-7-carboxylate (37.0 mg).

LC-MS (ESI): m/z=565.4 [M+H]⁺.

### Preparation of 3-(6-(4-((7-azaspiro[3.5]nonan-2-yl)methyl)piperazin-1-yl)-1-methyl-1H-indazol-3-yl)piperidine-2,6-dione

A solution of tert-butyl 2-((4-(3-(2,6-dioxopiperidin-3-yl)-1-methyl-1H-indazol-6-yl)piperazin-1-yl)methyl)-7-azaspiro[3.5]nonane-7-carboxylate (43.0 mg, 76.1 mol) in DCM (1.00 mL) was treated with a 1:1 solution of TFA (1.00 mL, 13.0 mmol) and DCM (1.00 mL). The reaction mixture was allowed to stir at room temperature for 18 h. The reaction mixture was concentrated under reduced pressure. The residue was suspended in DCM, and the mixture was concentrated again under reduced pressure to afford 3-(6-(4-((7-azaspiro[3.5]nonan-2-yl)methyl)piperazin-1-yl)-1-methyl-1H-indazol-3-yl)piperidine-2,6-dione (52.7 mg).

LC-MS (ESI): m/z=465.3 [M+H]⁺.

### Preparation of 3-(6-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-7-azaspiro[3.5]nonan-2-yl)methyl)piperazin-1-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (52.3 mg, 112 mol) and 3-(6-(4-((7-azaspiro[3.5]nonan-2-yl)methyl)piperazin-1-yl)-1-methyl-1H-indazol-3-yl)piperidine-2,6-dione (52.7 mg, 76.1 mol) in DMSO (1.00 mL) was treated with DIEA (69.2 μL, 397 mol). The reaction mixture was warmed to 80° C. for 18 h. The reaction mixture was cooled to room temperature, filtered, and purified by prep-HPLC to afford 3-(6-(4-((7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-7-azaspiro[3.5]nonan-2-yl)methyl)piperazin-1-yl)-1-methyl-1H-indazol-3-yl)piperidine-2,6-dione (28.5 mg).

¹H NMR (400 MHz, DMSO-d₆) δ 10.84 (s, 1H), 8.78 (s, 1H), 8.18 (d, J=2.3 Hz, 1H), 8.01 (s, 1H), 7.71 (dd, J=9.0, 2.1 Hz, 1H), 7.49 (d, J=8.9 Hz, 1H), 7.43 (d, J=9.1 Hz, 1H), 6.90 (dd, J=9.0, 1.9 Hz, 1H), 6.83 (s, 1H), 6.19 (d, J=4.3 Hz, 1H), 4.51-4.32 (m, 2H), 4.25 (dd, J=9.2, 5.1 Hz, 1H), 3.88 (s, 3H), 3.62-3.51 (m, 5H), 3.51-3.44 (m, 2H), 3.26-3.10 (m, 5H), 2.72-2.57 (m, 2H), 2.56-2.53 (m, 2H), 2.49-2.37 (m, 3H), 2.35-2.24 (m, 1H), 2.22-2.09 (m, 1H), 2.03-1.88 (m, 3H), 1.55 (t, J=5.7 Hz, 2H), 1.50-1.38 (m, 5H), 1.38-1.27 (m, 1H), 0.77-0.66 (m, 1H), 0.57-0.45 (m, 2H), 0.43-0.29 (m, 1H).

LC-MS (ESI): m/z=896.5 [M+H]⁺.

### Example 149. 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)sulfonyl)piperazin-1-yl)-1-methyl-1H-indazol-3-yl)piperidine-2,6-dione (Compound 478a)

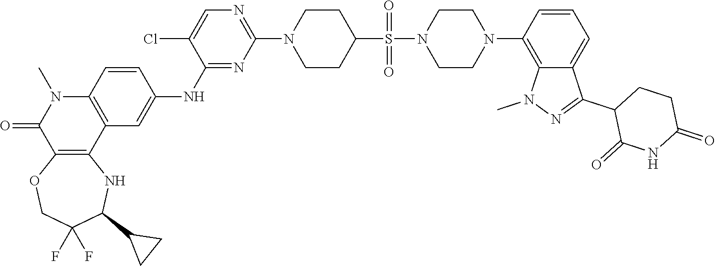 [SMILES: Cn1nc(C2CCC(=O)NC2=O)c2cccc(N3CCN(S(=O)(=O)C4CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-((4-(3-(2,6-dioxopiperidin-3-yl)-1-methyl-1H-indazol-7-yl)piperazin-1-yl)sulfonyl)piperidine-1-carboxylate

To a solution of 3-(1-methyl-7-(piperazin-1-yl)-1H-indazol-3-yl)piperidine-2,6-dione, HCl (100 mg, 1 equiv., 275 mol) in N,N-dimethylformamide (1.0 mL) was added tert-butyl 4-(chlorosulfonyl)piperidine-1-carboxylate (85.8 mg, 1.1 equiv., 302 mol) and DIEA (107 mg, 144 μL, 3.0 equiv., 825 mol). The reaction mixture was heated to 80° C. for 24 h. Additional tert-butyl 4-(chlorosulfonyl)piperidine-1-carboxylate (85.8 mg, 1.1 equiv., 302 mol) was added, and stirring was continued for an additional 24 h. The reaction mixture was partitioned between ethyl acetate and water. The organic layer was removed, and the aqueous layer was extracted with ethyl acetate twice more. The combined organic layer was washed with brine, dried over sodium sulfate and filtered. The filtrate was concentrated under reduced pressure to give a yellow oil which was purified by silica gel chromatography (0-20% methanol in dichloromethane) to afford tert-butyl 4-((4-(3-(2,6-dioxopiperidin-3-yl)-1-methyl-1H-indazol-7-yl)piperazin-1-yl)sulfonyl)piperidine-1-carboxylate (50 mg) as a foamy yellow solid.

LC-MS (ESI): m/z=575.3 [M+1]⁺

### Preparation of 3-(1-methyl-7-(4-(piperidin-4-ylsulfonyl)piperazin-1-yl)-1H-indazol-3-yl)piperidine-2,6-dione, HCl

To a solution of tert-butyl 4-((4-(3-(2,6-dioxopiperidin-3-yl)-1-methyl-1H-indazol-7-yl)piperazin-1-yl)sulfonyl)piperidine-1-carboxylate (50 mg, 1 equiv, 87 mol) in 1,4-dioxane (1.0 mL) was added HCl (72.9 mg, 500 μL, 4.0 M, 23 equiv., 2.00 mmol). The reaction mixture was stirred at ambient temperature for 2 h. The reaction mixture was concentrated under reduced pressure to afford 3-(1-methyl-7-(4-(piperidin-4-ylsulfonyl)piperazin-1-yl)-1H-indazol-3-yl)piperidine-2,6-dione, HCl (36 mg) as an off-white solid.

LC-MS (ESI): m/z=475.1 [M+1]⁺

### Preparation of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)sulfonyl)piperazin-1-yl)-1-methyl-1H-indazol-3-yl)piperidine-2,6-dione

To a solution of 3-(1-methyl-7-(4-(piperidin-4-ylsulfonyl)piperazin-1-yl)-1H-indazol-3-yl)piperidine-2,6-dione, HCl (36 mg, 1 equiv, 70 mol) in DMSO (1.0 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (33 mg, 1 equiv., 70 mol) and DIEA (46 mg, 61 μL, 5.0 equiv., 0.35 mmol). The reaction mixture was heated to 100° C. for 90 min. The reaction mixture was filtered and purified by reverse-phase HPLC to afford 3-(7-(4-((1-(5-chloro-4-(((S)-2-cyclopropyl-3,3-difluoro-7-methyl-6-oxo-1,2,3,4,6,7-hexahydro-[1,4]oxazepino [2,3-c]quinolin-10-yl)amino)pyrimidin-2-yl)piperidin-4-yl)sulfonyl)piperazin-1-yl)-1-methyl-1H-indazol-3-yl)piperidine-2,6-dione (23 mg) as an off-white solid.

¹H NMR (DMSO-d6, 400 MHz) δ 10.88 (s, 1H), 8.21 (d, 1H, J=2.0 Hz), 8.1-8.2 (m, 1H), 7.71 (dd, 1H, J=1.9, 9.1 Hz), 7.4-7.5 (m, 2H), 7.0-7.1 (m, 2H), 6.24 (br d, 1H, J=1.8 Hz), 4.5-4.6 (m, 2H), 4.3-4.5 (m, 3H), 4.2-4.3 (m, 3H), 3.67 (br s, 2H), 3.5-3.6 (m, 5H), 3.1-3.3 (m, 5H), 2.90 (br t, 2H, J=12.6 Hz), 2.7-2.8 (m, 2H), 2.6-2.7 (m, 2H), 2.3-2.4 (m, 1H), 2.1-2.2 (m, 1H), 2.0-2.1 (m, 2H), 1.5-1.6 (m, 2H), 1.3-1.4 (m, 1H), 0.6-0.8 (m, 1H), 0.4-0.6 (m, 2H), 0.3-0.4 (m, 1H).

LC-MS (ESI): m/z=906.5 [M+1]⁺

### Example 150. 3-(7-(4-(((R)-4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-(hydroxymethyl)piperazin-1-yl)methyl)piperidin-1-yl)-1-methyl-1H-indazol-3-yl)piperidine-2,6-dione (Compound 484a)

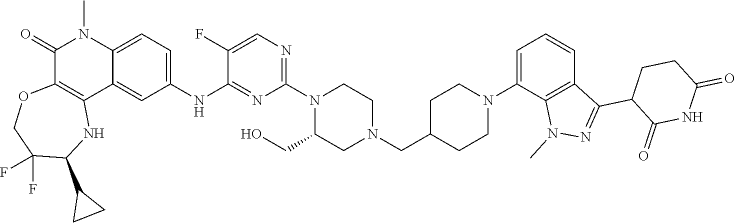 [SMILES: Cn1nc(C2CCC(=O)NC2=O)c2cccc(N3CCC(CN4CCN(c5ncc(F)c(Nc6ccc7c(c6)c6c(c(=O)n7C)OCC(F)(F)[C@H](C7CC7)N6)n5)[C@@H](CO)C4)CC3)c21]

### Preparation of 3-(7-(4-(((R)-4-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-(hydroxymethyl)piperazin-1-yl)methyl)piperidin-1-yl)-1-methyl-1H-indazol-3-yl)piperidine-2,6-dione

Prepared in a similar manner as Example 99. 3-(7-(4-(((R)-4-(4-(((S)-2-cyclopropyl-3,3-difluoro-7-methyl-6-oxo-1,2,3,4,6,7-hexahydro-[1,4]oxazepino [2,3-c]quinolin-10-yl)amino)-5-fluoropyrimidin-2-yl)-3-(hydroxymethyl)piperazin-1-yl)methyl)piperidin-1-yl)-1-methyl-1H-indazol-3-yl)piperidine-2,6-dione (8.7 mg).

¹H NMR (400 MHz, DMSO-d₆) δ 10.87 (s, 1H), 9.27 (s, 1H), 8.24 (d, J=2.3 Hz, 1H), 8.00 (d, J=3.6 Hz, 1H), 7.72 (dd, J=9.1, 2.2 Hz, 1H), 7.42 (d, J=9.1 Hz, 1H), 7.39-7.31 (m, 1H), 7.08-6.94 (m, 2H), 6.16 (s, 1H), 4.75 (s, 1H), 4.55-4.47 (m, 1H), 4.47-4.40 (m, 1H), 4.33 (dd, J=9.4, 5.2 Hz, 1H), 4.24 (s, 3H), 3.73 (t, J=9.0 Hz, 1H), 3.55 (s, 3H), 3.29-3.20 (m, 5H), 3.03 (t, J=12.6 Hz, 2H), 2.82 (d, J=10.6 Hz, 1H), 2.73-2.57 (m, 4H), 2.40-2.26 (m, 1H), 2.25-2.11 (m, 3H), 1.98-1.83 (m, 5H), 1.74-1.65 (m, 1H), 1.47-1.27 (m, 3H), 0.74-0.64 (m, 1H), 0.57-0.45 (m, 2H), 0.39-0.28 (m, 1H).

LC-MS (ESI): m/z=870.5 [M+H]⁺.

### Example 151: 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-hydroxypiperidin-4-yl)methyl)piperazin-1-yl)-1-methyl-1H-indazol-3-yl)piperidine-2,6-dione (Compound 489a)

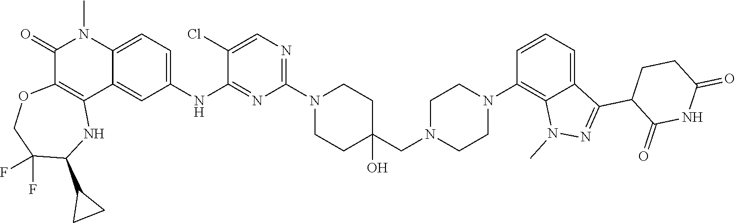 [SMILES: Cn1nc(C2CCC(=O)NC2=O)c2cccc(N3CCN(CC4(O)CCN(c5ncc(Cl)c(Nc6ccc7c(c6)c6c(c(=O)n7C)OCC(F)(F)[C@H](C7CC7)N6)n5)CC4)CC3)c21]

### Preparation of tert-butyl 4-((4-(3-(2,6-bis(benzyloxy)pyridin-3-yl)-1-methyl-1H-indazol-7-yl)piperazin-1-yl)methyl)-4-hydroxypiperidine-1-carboxylate

A solution of 3-(2,6-bis(benzyloxy)pyridin-3-yl)-1-methyl-7-(piperazin-1-yl)-1H-indazole, HCl (300 mg, 1 equiv., 553 mol) and tert-butyl 1-oxa-6-azaspiro[2.5]octane-6-carboxylate (236 mg, 2 equiv., 1.11 mmol) in EtOH (15 mL) was treated with K₂CO₃ (382 mg, 5 equiv., 2.77 mmol).

The reaction mixture was warmed at 130° C. for 60 minutes in a microwave reactor. The reaction mixture was cooled to room temperature and was filtered and concentrated to afford tert-butyl 4-((4-(3-(2,6-bis(benzyloxy)pyridin-3-yl)-1-methyl-1H-indazol-7-yl)piperazin-1-yl)methyl)-4-hydroxypiperidine-1-carboxylate (398 mg, 554 mol, 100%) which was used directly in the next step without further purification.

LC-MS (ESI): m/z=719.6 [M+H]⁺

### Preparation of tert-butyl 4-((4-(3-(2,6-dioxopiperidin-3-yl)-1-methyl-1H-indazol-7-yl)piperazin-1-yl)methyl)-4-hydroxypiperidine-1-carboxylate

A solution of tert-butyl 4-((4-(3-(2,6-bis(benzyloxy)pyridin-3-yl)-1-methyl-1H-indazol-7-yl)piperazin-1-yl)methyl)-4-hydroxypiperidine-1-carboxylate (398 mg, 1 equiv., 554 mol) and Pd(OH)₂ on carbon (150 mg, 20% wt, 0.386 equiv., 214 mol) in DMF (2 mL)/iPrOH (6 mL) at 50° C. was bubbled with hydrogen for 30 minutes. The reaction mixture was stirred under a hydrogen atmosphere at 50° C. for 18 hours. Pd(OH)₂ on carbon (150 mg, 20% Wt, 0.386 equiv., 214 mol) was added and the reaction mixture was bubbled with hydrogen at 50° C. for 30 min.

The reaction mixture was filtered through celite and washed with DMF (5 mL), EtOAc (50 mL), and iPrOH (10 mL). The resulting filtrate was concentrated and diluted with DMF (2 mL) and iPrOH (6 mL). To this solution was added Pd(OH)₂ on carbon (150 mg, 20% Wt, 0.386 equiv., 214 mol), and the reaction mixture was bubbled with hydrogen at 50° C. for 30 minutes following by stirring under a hydrogen atmosphere for 2 h. The reaction mixture was filtered through celite, and the filtrate was concentrated and purified by reverse-phase FCC to afford tert-butyl 4-((4-(3-(2,6-dioxopiperidin-3-yl)-1-methyl-1H-indazol-7-yl)piperazin-1-yl)methyl)-4-hydroxypiperidine-1-carboxylate (136 mg, 252 mol, 45.4%).

LC-MS (ESI): m/z=541.4 [M+H]⁺

### Preparation of 3-(7-(4-((4-hydroxypiperidin-4-yl)methyl)piperazin-1-yl)-1-methyl-1H-indazol-3-yl)piperidine-2,6-dione

A solution of tert-butyl 4-((4-(3-(2,6-dioxopiperidin-3-yl)-1-methyl-1H-indazol-7-yl)piperazin-1-yl)methyl)-4-hydroxypiperidine-1-carboxylate, formic acid (136 mg, 1 equiv., 232 mol) in 1,4-dioxane (2 mL) was treated with HCl (169 mg, 1.16 mL, 4 molar, 20 equiv., 4.64 mmol) (4 M in dioxane) at room temperature. The reaction mixture was stirred for 30 minutes. The volatiles were removed under a stream of nitrogen, and the resulting solid was further dried under vacuum to afford 3-(7-(4-((4-hydroxypiperidin-4-yl)methyl)piperazin-1-yl)-1-methyl-1H-indazol-3-yl)piperidine-2,6-dione (102 mg, 232 mol, 99.9%) as a white solid.

LC-MS (ESI): m/z=441.5 [M+H]⁺

### Preparation of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-hydroxypiperidin-4-yl)methyl)piperazin-1-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (81 mg, 1.5 equiv., 0.17 mmol), 3-(7-(4-((4-hydroxypiperidin-4-yl)methyl)piperazin-1-yl)-1-methyl-1H-indazol-3-yl)piperidine-2,6-dione (51 mg, 1.00 equiv, 0.12 mmol) and CsF (53 mg, 3.00 equiv, 0.35 mmol) in DMSO (2 mL) was treated with DIEA (45 mg, 60 μL, 3.00 equiv., 0.35 mmol). The reaction mixture was warmed to 80° C. for 16 h. The reaction mixture was filtered and was purified by HPLC to afford 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-4-hydroxypiperidin-4-yl)methyl)piperazin-1-yl)-1-methyl-1H-indazol-3-yl)piperidine-2,6-dione (41 mg, 47 mol, 41%).

LC-MS (ESI): m/z=872.6 [M+H]⁺

¹H NMR (400 MHz, DMSO) δ 10.87 (s, 1H), 8.74 (s, 1H), 8.16 (d, J=2.3 Hz, 1H), 8.02 (s, 1H), 7.79-7.73 (m, 1H), 7.45 (d, J=9.1 Hz, 1H), 7.41-7.34 (m, 1H), 7.06-6.96 (m, 2H), 6.17 (s, 1H), 4.50-4.36 (m, 1H), 4.36-4.29 (m, 1H), 4.23 (s, 3H), 4.14-4.02 (m, 2H), 3.56 (s, 3H), 3.31-3.18 (m, 4H), 3.08-2.57 (m, 7H), 2.38-2.27 (m, 3H), 2.21-2.11 (m, 1H), 1.54-1.44 (m, 5H), 1.37-1.27 (m, 1H), 0.74-0.65 (m, 1H), 0.57-0.47 (m, 2H), 0.40-0.30 (m, 1H).

### Example 152. 3-(7-(4-(7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methyl-5,6,7,8-tetrahydroimidazo[1,5-a]pyrazin-1-yl)piperidin-1-yl)-1-methyl-1H-indazol-3-yl)piperidine-2,6-dione (Compound 490a)

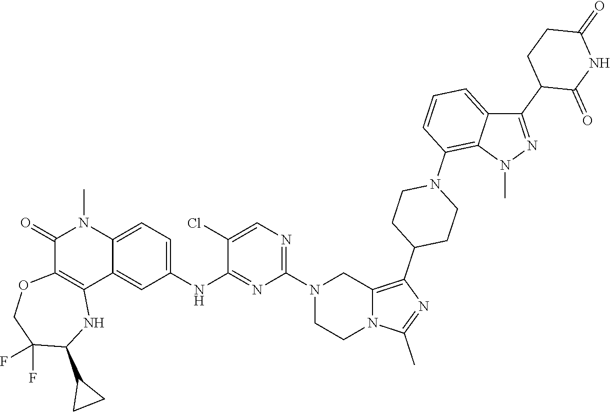 [SMILES: Cc1nc(C2CCN(c3cccc4c(C5CCC(=O)NC5=O)nn(C)c34)CC2)c2n1CCN(c1ncc(Cl)c(Nc3ccc4c(c3)c3c(c(=O)n4C)OCC(F)(F)[C@H](C4CC4)N3)n1)C2]

### Preparation of tert-butyl 1-(1-(3-(2,6-bis(benzyloxy)pyridin-3-yl)-1-methyl-1H-indazol-7-yl)piperidin-4-yl)-3-methyl-5,6-dihydroimidazo[1,5-a]pyrazine-7(8H)-carboxylate

A mixture of tert-butyl 3-methyl-1-(piperidin-4-yl)-5,6-dihydroimidazo[1,5-a]pyrazine-7(8H)-carboxylate hydrochloride (200 mg, 560 mol), 3-(2,6-bis(benzyloxy)pyridin-3-yl)-7-bromo-1-methyl-1H-indazole (322 mg, 644 mol), and RuPhos-Pd-G3 (46.9 mg, 56.0 mol) in THF (3.00 mL) was sparged with N_{2(g)} for 5 min and then treated with sodium tert-butoxide (2.0 M, 1.12 mL, 2.24 mmol). The reaction mixture was warmed to 70° C. for 2 h and then allowed to cool to room temperature and stand overnight. The reaction mixture was treated with formic acid (84.6 μL, 2.24 mmol), diluted with DCM, and dry loaded and was purified by column chromatography (0-20% DCM/MeOH) to afford tert-butyl 1-(1-(3-(2,6-bis(benzyloxy)pyridin-3-yl)-1-methyl-1H-indazol-7-yl)piperidin-4-yl)-3-methyl-5,6-dihydroimidazo[1,5-a]pyrazine-7(8H)-carboxylate (99 mg).

LC-MS (ESI): m/z=740.5 [M+H]⁺.

### Preparation of tert-butyl 1-(1-(3-(2,6-dioxopiperidin-3-yl)-1-methyl-1H-indazol-7-yl)piperidin-4-yl)-3-methyl-5,6-dihydroimidazo[1,5-a]pyrazine-7(8H)-carboxylate

A solution of tert-butyl 1-(1-(3-(2,6-bis(benzyloxy)pyridin-3-yl)-1-methyl-1H-indazol-7-yl)piperidin-4-yl)-3-methyl-5,6-dihydroimidazo[1,5-a]pyrazine-7(8H)-carboxylate (93.6 mg, 127 mol) in THF (1.00 mL) and DMF (1.00 mL) was sparged with N_{2(g)} for 5 minutes, treated with Pd(OH)₂ on carbon (54.2 mg, 20% Wt, 77.2 mol), and then sparged with H_{2(g)} for 5 min.

The reaction mixture was then warmed to 50° C. and stirred under an atmosphere of H_{2(g)} for 5 h.

The reaction mixture was sparged with N₂(g) and filtered through a pad of celite. the filter pad was rinsed with isopropanol/THF (1:1), and the filtrate was concentrated under reduced pressure. The crude residue was dissolved in DMSO, filtered, and purified by prep-HPLC to afford tert-butyl 1-(1-(3-(2,6-dioxopiperidin-3-yl)-1-methyl-1H-indazol-7-yl)piperidin-4-yl)-3-methyl-5,6-dihydroimidazo[1,5-a]pyrazine-7(8H)-carboxylate (13.4 mg).

LC-MS (ESI): m/z=562.3 [M+H]⁺.

### Preparation of 3-(1-methyl-7-(4-(3-methyl-5,6,7,8-tetrahydroimidazo[1,5-a]pyrazin-1-yl)piperidin-1-yl)-1H-indazol-3-yl)piperidine-2,6-dione

A solution of tert-butyl 1-(1-(3-(2,6-dioxopiperidin-3-yl)-1-methyl-1H-indazol-7-yl)piperidin-4-yl)-3-methyl-5,6-dihydroimidazo[1,5-a]pyrazine-7(8H)-carboxylate (13.4 mg, 23.9 mol) in DCM (1.00 mL) was treated with TFA (0.500 mL, 6.49 mmol). The reaction mixture was stirred at room temperature for 1.5 hours. The reaction mixture was concentrated under reduced pressure to afford 3-(1-methyl-7-(4-(3-methyl-5,6,7,8-tetrahydroimidazo[1,5-a]pyrazin-1-yl)piperidin-1-yl)-1H-indazol-3-yl)piperidine-2,6-dione (13.4 mg), which was used as is without further purification.

LC-MS (ESI): m/z=462.4 [M+H]⁺.

### Preparation of 3-(7-(4-(7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methyl-5,6,7,8-tetrahydroimidazo[1,5-a]pyrazin-1-yl)piperidin-1-yl)-1-methyl-1H-indazol-3-yl)piperidine-2,6-dione

A solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (24.2 mg, 1.62 equiv., 51.7 mol), 3-(1-methyl-7-(4-(3-methyl-5,6,7,8-tetrahydroimidazo[1,5-a]pyrazin-1-yl)piperidin-1-yl)-1H-indazol-3-yl)piperidine-2,6-dione (14.7 mg, 1.00 equiv., 31.8 mol), and CsF (14.5 mg, 3.00 equiv., 95.5 mol) in DMSO (0.700 mL) was treated with DIEA (16.5 mg, 22.2 μL, 4.00 equiv., 127 mol). The reaction mixture was warmed to 80° C. for 18 h. The reaction mixture was cooled to room temperature, filtered, and purified by prep-HPLC afford 3-(7-(4-(7-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methyl-5,6,7,8-tetrahydroimidazo[1,5-a]pyrazin-1-yl)piperidin-1-yl)-1-methyl-1H-indazol-3-yl)piperidine-2,6-dione (9.6 mg).

¹H NMR (400 MHz, DMSO-d₆) δ 10.88 (s, 1H), 8.99 (s, 1H), 8.20 (s, 1H), 8.13 (s, 1H), 7.71 (d, J=8.9 Hz, 1H), 7.47-7.31 (m, 2H), 7.04 (t, J=7.6 Hz, 1H), 6.94 (s, 1H), 6.33 (s, 1H), 4.81-4.66 (m, 2H), 4.52-4.37 (m, 2H), 4.34 (dd, J=9.5, 5.1 Hz, 1H), 4.27-4.14 (m, 4H), 4.06-3.91 (m, 1H), 3.91-3.75 (m, 2H), 3.61-3.38 (m, 3H), 3.23-2.99 (m, 6H), 2.71-2.58 (m, 3H), 2.39-2.27 (m, 1H), 2.24-2.12 (m, 4H), 2.00-1.73 (m, 1H), 1.72-1.43 (m, 2H), 1.38-1.19 (m, 1H), 0.70-0.66 (m, 1H), 0.55-0.39 (m, 2H), 0.33-0.17 (m, 1H).

LC-MS (ESI): m/z=893.5 [M+H]⁺.

### Example 153: 3-(7-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione

### [3-(7-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione](Compound 499a)

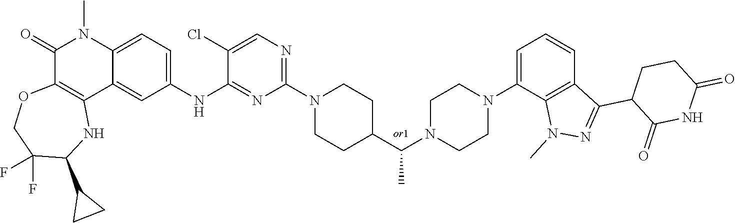 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCN(c2cccc3c(C4CCC(=O)NC4=O)nn(C)c23)CC1]

### Preparation of benzyl 4-(1-(1-(tert-butoxycarbonyl)piperidin-4-yl)ethyl)piperazine-1-carboxylate

To a stirred solution of benzyl piperazine-1-carboxylate (20.0 g, 90.79 mmol) and tert-butyl 4-acetylpiperidine-1-carboxylate (41.2 g, 181.59 mmol) in THF (400 mL) was added Ti(O-iPr) (20.0 mL). The mixture was stirred for 1 h followed by the addition of sodium cyanoborohydride (3.83 g, 18.18 mmol) under a nitrogen atmosphere. The mixture was stirred at room temperature for 3 h. The reaction mixture was quenched with water (400 mL) and extracted with DCM (200.0 mL). The organic layer was separated, washed with brine solution (100 mL), dried over anhydrous Na₂SO₄, filtered, and dried under vacuum to get a crude product which was purified by flash column chromatography (30% ethyl acetate in petroleum ether) to afford benzyl 4-(1-(1-(tert-butoxycarbonyl)piperidin-4-yl)ethyl)piperazine-1-carboxylate (8 g) as an off white solid.

LC-MS (ESI): m/z=432.54 [M+H]⁺

### Preparation of benzyl (R)-4-(1-(1-(tert-butoxycarbonyl)piperidin-4-yl)ethyl)piperazine-1-carboxylate and benzyl (S)-4-(1-(1-(tert-butoxycarbonyl)piperidin-4-yl)ethyl)piperazine-1-carboxylate

Stereoisomers of benzyl 4-(1-(1-(tert-butoxycarbonyl)piperidin-4-yl)ethyl)piperazine-1-carboxylate were separated by SFC to afford benzyl (R)-4-(1-(1-(tert-butoxycarbonyl)piperidin-4-yl)ethyl)piperazine-1-carboxylate (2.8 g) and benzyl (S)-4-(1-(1-(tert-butoxycarbonyl)piperidin-4-yl)ethyl)piperazine-1-carboxylate (2.8 g) as oils.

LC-MS (ESI): m/z=432.58 [M+H]⁺

LC-MS (ESI): m/z=432.32 [M+H]⁺

### Preparation of tert-butyl (R)-4-(1-(piperazin-1-yl)ethyl)piperidine-1-carboxylate

To a stirred solution of benzyl (R)-4-(1-(1-(tert-butoxycarbonyl)piperidin-4-yl)ethyl)piperazine-1-carboxylate (2.8 g, 6.48 mmol) in THF (84 mL) was added 20% Pd(OH)₂/C, H₂ (2.8 g, 100% w/w). The reaction mixture was degassed with argon for 10 minutes. The reaction mixture was stirred under hydrogen pressure (80 psi) at room temperature for 12 h. The reaction mixture was diluted with THF (50 mL) and filtered through a celite bed and washed with THF:DCM (1:1, 100 mL). The filtrate was concentrated and dried to get tert-butyl (R)-4-(1-(piperazin-1-yl)ethyl)piperidine-1-carboxylate (1.8 g) as a brown semi-solid which was used in the next step without further purification.

LC-MS (ESI): m/z=298.20 [M+H]⁺

### Preparation of tert-butyl (R)-4-(1-(4-(3-(2,6-bis(benzyloxy)pyridin-3-yl)-1-methyl-1H-indazol-7-yl)piperazin-1-yl)ethyl)piperidine-1-carboxylate

To a stirred solution of tert-butyl (R)-4-(1-(piperazin-1-yl)ethyl)piperidine-1-carboxylate (1.0 g, 3.36 mmol) and 3-(2,6-bis(benzyloxy)pyridin-3-yl)-7-bromo-1-methyl-1H-indazole (0.841 g, 1.68 mmol) in 1,4-dioxane (40 mL) was added cesium carbonate (3.28 g, 10.0 mmol). The reaction mixture was degassed with argon for 10 minutes. To this mixture was added Pd-PEPPSI-iHept-C₁ (0.16 g, 0.168 mmol). The reaction mixture was stirred at 100° C. for 12 h. The reaction mixture was cooled to room temperature and filtered through a celite bed, and the filtrate was concentrated to get the crude product which was purified by flash column chromatography (30% ethyl acetate in petroleum ether) to afford tert-butyl (R)-4-(1-(4-(3-(2,6-bis(benzyloxy)pyridin-3-yl)-1-methyl-1H-indazol-7-yl)piperazin-1-yl)ethyl)piperidine-1-carboxylate (0.7 g) as an off white solid.

LC-MS (ESI): m/z=717.71 [M+H]⁺

### Preparation of tert-butyl 4-((1R)-1-(4-(3-(2,6-dioxopiperidin-3-yl)-1-methyl-1H-indazol-7-yl)piperazin-1-yl)ethyl)piperidine-1-carboxylate

To a stirred solution of tert-butyl (R)-4-(1-(4-(3-(2,6-bis(benzyloxy)pyridin-3-yl)-1-methyl-1H-indazol-7-yl)piperazin-1-yl)ethyl)piperidine-1-carboxylate (0.7 g, 0.976 mmol) in THF (28 mL) was added 20% Pd(OH)₂ (0.7 g). The reaction mixture was stirred under hydrogen pressure (80 psi) at room temperature for 12 h. The reaction mixture was diluted with THF (100 mL), filtered through a celite bed, and washed with THF:DCM (1:1, 200 mL). The filtrate was concentrated and dried to afford tert-butyl 4-((1R)-1-(4-(3-(2,6-dioxopiperidin-3-yl)-1-methyl-1H-indazol-7-yl)piperazin-1-yl)ethyl)piperidine-1-carboxylate (0.4 g) as a brown semi-solid which was used in the next step without further purification.

LC-MS (ESI): m/z=539.67 [M+H]⁺

### Preparation of 3-(1-methyl-7-(4-((R)-1-(piperidin-4-yl)ethyl)piperazin-1-yl)-1H-indazol-3-yl)piperidine-2,6-dione

To a stirred solution of tert-butyl 4-((1R)-1-(4-(3-(2,6-dioxopiperidin-3-yl)-1-methyl-1H-indazol-7-yl)piperazin-1-yl)ethyl)piperidine-1-carboxylate (0.4 g, 0.743 mmol) in DCM (6.0 mL) cooled to 0° C. was added TFA (2.0 mL). The mixture was stirred at room temperature for 3 h. The reaction mixture was concentrated to get the crude product which was triturated with diethyl ether (3 times) to afford 3-(1-methyl-7-(4-((R)-1-(piperidin-4-yl)ethyl)piperazin-1-yl)-1H-indazol-3-yl)piperidine-2,6-dione (0.4 g) as a brown solid.

LC-MS (ESI): m/z=439.29 [M+H]⁺

### Preparation of 3-(7-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione

### [3-(7-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione]

To a stirred solution of 3-(1-methyl-7-(4-((R)-1-(piperidin-4-yl)ethyl)piperazin-1-yl)-1H-indazol-3-yl)piperidine-2,6-dione (0.15 g, 0.342 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.171 mmol) in DMSO (4.5 mL) was added N,N-diisopropylethylamine (0.59 mL, 3.420 mmol). The resulting reaction mixture was stirred at 120° C. for 16 h. The reaction mixture was added to crushed ice and stirred for 30 min to precipitate a solid. The solid was filtered and dried to obtain a crude product which was purified by prep-HPLC, to afford 3-(7-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione [3-(7-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione](49 mg) as an off white solid.

LC-MS (ESI): m/z=870.46 [M+H]⁺

¹H NMR (400 MHz, DMSO-d6): δ 10.89 (s, 1H), 8.98 (br s, 1H) 8.90 (s, 1H), 8.16 (s, 1H), 8.06 (s, 1H), 7.73 (dd, J=7.2, 1.6 Hz, 1H), 7.49 (d, J=7.2 Hz, 1H), 7.43 (d, J=9.2 Hz, 1H), 7.09-7.00 (m, 2H), 6.26 (s, 1H), 4.55 (d, J=10.4 Hz, 1H), 4.50-4.30 (m, 3H), 4.25 (s, 3H), 3.50-3.30 (m, 8H), 3.33-3.10 (m, 5H), 2.83 (t, J=3.1 Hz, 2H), 2.70-2.65 (m, 2H) 2.36-2.32 (m, 3H), 1.68 (d, J=12 Hz, 1H), 1.59 (d, J=11.2 Hz, 1H), 1.40-1.35 (m, 2H), 1.30-1.25 (m, 4H), 0.72-0.75 (m, 1H), 0.53-0.50 (m, 2H), 0.40-0.35 (m, 1H).

### Example 154: 3-(7-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)-1-hydroxyethyl)piperidin-1-yl)-1-methyl-1H-indazol-3-yl)piperidine-2,6-dione

### [3-(7-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)-1-hydroxyethyl)piperidin-1-yl)-1-methyl-1H-indazol-3-yl)piperidine-2,6-dione](Compound

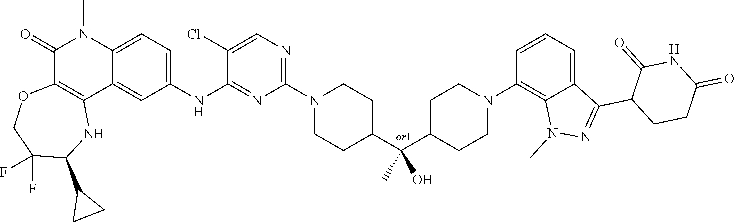 [SMILES: Cn1nc(C2CCC(=O)NC2=O)c2cccc(N3CCC([C@@](C)(O)C4CCN(c5ncc(Cl)c(Nc6ccc7c(c6)c6c(c(=O)n7C)OCC(F)(F)[C@H](C7CC7)N6)n5)CC4)CC3)c21]

### Preparation of benzyl 4-((pyridin-2-ylthio)carbonyl)piperidine-1-carboxylate

To a stirred solution of 1-((benzyloxy)carbonyl)piperidine-4-carboxylic acid (10.0 g, 37.98 mmol) and 1,2-di(pyridin-2-yl)disulfane (9.2 g, 41.77 mmol) in MeCN (200 mL) was added triphenylphosphine (10.90 g, 41.77 mmol). The mixture was stirred at room temperature for 16 h.

The reaction mixture was concentrated under reduced pressure to get a crude product which was purified by flash column chromatography (20% ethyl acetate in petroleum ether) to afford benzyl 4-((pyridin-2-ylthio)carbonyl)piperidine-1-carboxylate as a yellow liquid (8.0 g).

LC-MS (ESI): m/z=357.40 [M+H]⁺

### Preparation of benzyl 4-(1-(tert-butoxycarbonyl)piperidine-4-carbonyl)piperidine-1-carboxylate

To a stirred solution of benzyl 4-((pyridin-2-ylthio)carbonyl)piperidine-1-carboxylate (8.0 g, 22.44 mmol), 1-(tert-butyl) 4-(1,3-dioxoisoindolin-2-yl) piperidine-1,4-dicarboxylate (8.40 g, 22.44 mmol) and dimethyl [2,2′-bipyridine]-4,4′-dicarboxylate (0.12 g, 0.45 mmol) in THF/DMA (160 mL, 1:1) were added NiBr₂(dme) (0.14 g, 0.45 mmol), ZnCl₂ (0.61 g, 4.48 mmol), and zinc dust (2.93 g, 44.88 mmol). The reaction mixture was stirred at room temperature for 16 h. After completion of the reaction, the reaction mixture was filtered through celite and washed with ethyl acetate (50 mL). The filtrate was evaporated to get a crude product which was purified by column chromatography (50% ethyl acetate in petroleum ether) to afford benzyl 4-(1-(tert-butoxycarbonyl)piperidine-4-carbonyl)piperidine-1-carboxylate (8.0 g).

LC-MS (ESI): m/z=453.62 [M+22+H]⁺

### Preparation of benzyl 4-(piperidine-4-carbonyl)piperidine-1-carboxylate

To a stirred solution of benzyl 4-(1-(tert-butoxycarbonyl)piperidine-4-carbonyl)piperidine-1-carboxylate (6.0 g, 13.93 mmol) in DCM (120 mL) was added TFA (30.0 mL) at 0° C. The mixture was stirred at room temperature for 5 h. After completion, the reaction mixture was concentrated under reduced pressure, diluted with water (20 mL), and extracted with ethyl acetate (20 mL). After that, the aqueous layer was basified (to pH=12) by saturated solution of sodium hydroxide (20 mL) and extracted with ethyl acetate (3×20 mL). The organic was evaporated to get crude benzyl 4-(piperidine-4-carbonyl)piperidine-1-carboxylate (4.0 g) as a pale yellow liquid which was used in the next step without further purification.

LC-MS (ESI): m/z=331.18 [M+H]⁺

### Preparation of benzyl 4-(1-(3-(2,6-bis(benzyloxy)pyridin-3-yl)-1-methyl-1H-indazol-7-yl)piperidine-4-carbonyl)piperidine-1-carboxylate

A stirred solution of benzyl 4-(piperidine-4-carbonyl)piperidine-1-carboxylate (2.64 g, 7.99 mmol), 3-(2,6-bis(benzyloxy)pyridin-3-yl)-7-bromo-1-methyl-1H-indazole (2.0 g, 3.99 mmol), and Cs₂CO₃ (3.89 g, 11.99 mmol) in 1,4-dioxane (40 mL) was degassed with N₂ for 5 min. Afterwards, Pd-PEPSI-iHEPT-Cl (0.19 g, 0.2 mmol) was added, and the reaction mixture was heated at 100° C. and stirred for 16 h. After completion, the reaction was filtered through celite, washed with ethyl acetate and evaporated to get crude a compound which was purified by column chromatography (50% ethyl acetate in petroleum ether) to afford benzyl 4-(1-(3-(2,6-bis(benzyloxy)pyridin-3-yl)-1-methyl-1H-indazol-7-yl)piperidine-4-carbonyl)piperidine-1-carboxylate (1.8 g) as a white solid.

LC-MS (ESI): m/z=750.68 [M+H]⁺

### Preparation of benzyl (S)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate and benzyl (R)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate

To a stirred solution of benzyl 4-(1-(3-(2,6-bis(benzyloxy)pyridin-3-yl)-1-methyl-1H-indazol-7-yl)piperidine-4-carbonyl)piperidine-1-carboxylate (2.0 g, 2.66 mmol) in THF (60 mL) was added methyl magnesium chloride (3.0 M in diethyl ether) (4.43 mL, 13.33 mmol) at −60° C. The mixture was allowed to stir at room temperature for 5 h. After completion, the reaction mixture was quenched with NH₄Cl (20 mL) and extracted with ethyl acetate (2×100 mL). The organic layer was evaporated to get crude product which was purified by column chromatography (50% ethyl acetate in petroleum ether) to afford benzyl 4-(1-(1-(3-(2,6-bis(benzyloxy)pyridin-3-yl)-1-methyl-1H-indazol-7-yl)piperidin-4-yl)-1-hydroxyethyl)piperidine-1-carboxylate (1.8 g) as a white solid. Stereoisomers of benzyl 4-(1-(1-(3-(2,6-bis(benzyloxy)pyridin-3-yl)-1-methyl-1H-indazol-7-yl)piperidin-4-yl)-1-hydroxyethyl)piperidine-1-carboxylate were separated by SFC to benzyl (S)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate, and benzyl (R)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate

LC-MS (ESI): m/z=766.67 [M+H]⁺

### Preparation of 3-(7-(4-((R)-1-hydroxy-1-(piperidin-4-yl)ethyl)piperidin-1-yl)-1-methyl-1H-indazol-3-yl)piperidine-2,6-dione

To a stirred solution of benzyl (R)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate (0.6 g, 0.78 mmol) in THF (18.0 mL) was added 20% palladium hydroxide on carbon (1.8 g, 100% w/w). The reaction mixture was stirred under H₂ pressure (80 Psi) at room temperature for 16 h. After completion, the reaction mixture was filtered through celite and washed with DCM (50 mL). The filtrate was evaporated to get a crude compound which was triturated with diethyl ether to afford 3-(7-(4-((R)-1-hydroxy-1-(piperidin-4-yl)ethyl)piperidin-1-yl)-1-methyl-1H-indazol-3-yl)piperidine-2,6-dione (0.15 g) as a white solid.

### Preparation of 3-(7-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)-1-hydroxyethyl)piperidin-1-yl)-1-methyl-1H-indazol-3-yl)piperidine-2,6-dione

### [3-(7-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)-1-hydroxyethyl)piperidin-1-yl)-1-methyl-1H-indazol-3-yl)piperidine-2,6-dione]

To a stirred solution of 3-(7-(4-((R)-1-hydroxy-1-(piperidin-4-yl)ethyl) piperidin-1-yl)-1-methyl-1H-indazol-3-yl)piperidine-2,6-dione (0.1 g, 0.22 mmol) and DIPEA (0.4 mL, 2.20 mmol) in DMSO (3.0 mL) was added (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.051 g, 0.11 mmol). The mixture was heated at 100° C. and stirred for 5 h. The reaction was concentrated under reduced pressure to get a crude product which was purified by prep-HPLC to afford 3-(7-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)-1-hydroxyethyl)piperidin-1-yl)-1-methyl-1H-indazol-3-yl)piperidine-2,6-dione [3-(7-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)-1-hydroxyethyl)piperidin-1-yl)-1-methyl-1H-indazol-3-yl)piperidine-2,6-dione](22.9 mg) as a white solid.

¹H NMR (400 MHz, DMSO-d6): δ 10.87 (s, 1H), 8.89 (s, 1H), 8.19 (br s, 1H), 8.05 (s, 1H), 7.74 (d, J=9.2 Hz, 1H), 7.45 (d, J=9.0 Hz, 1H), 7.37-7.33 (m, 2H), 7.01-6.95 (m, 2H), 6.19 (br s, 1H), 4.60-4.21 (m, 5H), 4.24 (s, 3H), 3.57 (s, 3H), 3.26 (s, 3H), 2.71-2.58 (m, 5H), 2.36-2.17 (m, 2H), 1.74-1.62 (m, 6H), 1.25-1.17 (m, 5H), 1.09 (t, J=6.8 Hz, 1H), 0.95 (s, 3H), 0.71-0.67 (m, 1H), 0.51-0.52 (m, 2H), 0.34-0.33 (m, 1H).

LC-MS (ESI): m/z=885.51 [M+H]⁺

### Example 155: 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,3-dimethylpiperazin-1-yl)-1-methyl-1H-indazol-3-yl)piperidine-2,6-dione (Compound 505a)

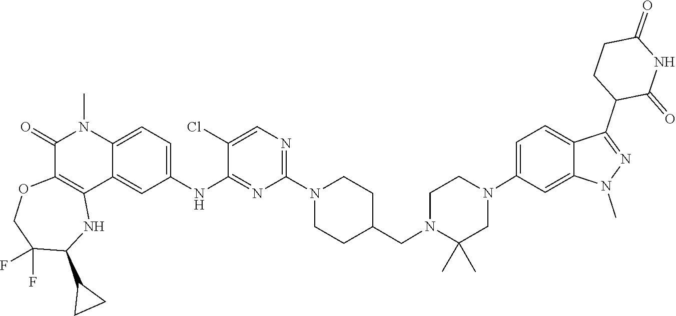 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)C(C)(C)C3)cc21]

### Preparation of tert-butyl 4-((1-((benzyloxy)carbonyl)piperidin-4-yl)methyl)-3,3-dimethylpiperidine-1-carboxylate

To a stirred solution of tert-butyl 3,3-dimethylpiperazine-1-carboxylate (1.0 g, 4.66 mmol) and benzyl 4-formylpiperidine-1-carboxylate (1.38 g, 5.60 mmol) in THF (10.0 m1) was added acetic acid (0.20 ml, 4.66 mmol) and anhydrous sodium acetate (1.14 g, 13.99 mmol). The reaction was stirred at room temperature for 1 h followed by the addition of sodium triacetoxyborohydride (2.96 g, 13.99 mmol) at 0° C. The reaction mixture was stirred at room temperature for 12 h. The reaction mixture was quenched with water (50 mL) and extracted with ethyl acetate (100 mL). The organic layer was separated and concentrated under reduced pressure to get a crude product which was purified by flash column chromatography (25% ethyl acetate in petroleum ether) to afford tert-butyl 4-((1-((benzyloxy)carbonyl)piperidin-4-yl)methyl)-3,3-dimethylpiperidine-1-carboxylate as a pure product (1.0 g).

LC-MS (ESI): m/z=446.57 [M+H]⁺

### Preparation of benzyl 4-((2,2-dimethylpiperazin-1-yl)methyl)piperidine-1-carboxylate

To a stirred solution of tert-butyl 4-((1-((benzyloxy)carbonyl)piperidin-4-yl)methyl)-3,3-dimethylpiperidine-1-carboxylate (2 g, 4.48 mmol) in DCM (20 mL) was added TFA (2 mL) at 0° C. The reaction mixture was stirred at room temperature for 3 h. The reaction mixture was concentrated under reduced pressure to get a crude product which was purified by trituration with diethyl ether (20 mL) to afford benzyl 4-((2,2-dimethylpiperazin-1-yl)methyl)piperidine-1-carboxylate (1.5 g) as a solid.

LC-MS (ESI): m/z=346.47 [M+H]⁺

### Preparation of benzyl 4-((4-(3-(2,6-bis(benzyloxy)pyridin-3-yl)-1-methyl-1H-indazol-6-yl)-2,2-dimethylpiperazin-1-yl)methyl)piperidine-1-carboxylate

To a stirred solution of benzyl 4-((2,2-dimethylpiperazin-1-yl)methyl)piperidine-1-carboxylate (1.0 g, 2.89 mmol), 3-(2,6-bis(benzyloxy)pyridin-3-yl)-6-bromo-1-methyl-1H-indazole (0.72 g, 1.44 mmol), and ^{t}BuONa (2 M in THF) (4.33 mL, 8.67 mmol) in 1-4 dioxane (10 mL) was added Ruphos-Pd-G3 (0.24 g, 0.28 mmol) under N₂. The reaction mixture was heated at 100° C. and stirred for 16 h. The reaction mixture was quenched with ice cold water (20 mL) and extracted with ethyl acetate (100 mL). The organic layer was separated and concentrated under reduced pressure to get crude product which was purified by flash column chromatography (50-60% ethyl acetate in petroleum ether) to afford benzyl 4-((4-(3-(2,6-bis(benzyloxy)pyridin-3-yl)-1-methyl-1H-indazol-6-yl)-2,2-dimethylpiperazin-1-yl)methyl)piperidine-1-carboxylate (0.53 g).

LC-MS (ESI): m/z=765.80 [M+H]⁺

### Preparation of 3-(6-(3,3-dimethyl-4-(piperidin-4-ylmethyl)piperazin-1-yl)-1-methyl-1H-indazol-3-yl)piperidine-2,6-dione

A stirred solution of benzyl 4-((4-(3-(2,6-bis(benzyloxy)pyridin-3-yl)-1-methyl-1H-indazol-6-yl)-2,2-dimethylpiperazin-1-yl)methyl)piperidine-1-carboxylate (0.80 g, 1.04 mmol) in THF (24 mL) was degassed with nitrogen for 5 min followed by the addition of 20% palladium hydroxide on carbon (0.80 g, 100% w/w). The mixture was stirred under hydrogen pressure (80 Psi) at room temperature for 16 h. The reaction mixture was diluted with ethyl acetate (50 mL), filtered through a celite bed, and concentrated to get a crude product which was purified by trituration with diethyl ether to afford 3-(6-(3,3-dimethyl-4-(piperidin-4-ylmethyl)piperazin-1-yl)-1-methyl-1H-indazol-3-yl)piperidine-2,6-dione (0.20 g) as a solid.

LC-MS (ESI): m/z=553.51 [M+H]⁺

### Preparation of 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,3-dimethylpiperazin-1-yl)-1-methyl-1H-indazol-3-yl)piperidine-2,6-dione

To a stirred solution of 3-(6-(3,3-dimethyl-4-(piperidin-4-ylmethyl)piperazin-1-yl)-1-methyl-1H-indazol-3-yl)piperidine-2,6-dione (0.100 g, 0.221 mmol) and (S)-2-cyclo propyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.052 g, 0.110 mmol) in DMSO (2 mL) was added DIPEA (0.10 mL, 0.663 mmol) at room temperature. The mixture was heated at 100° C. and stirred for 4 h. The reaction mixture was concentrated under reduced pressure and purified by prep-HPLC to afford 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,3-dimethylpiperazin-1-yl)-1-methyl-1H-indazol-3-yl)piperidine-2,6-dione (0.025 g) as an off white solid.

¹H NMR (400 MHz, DMSO-d6): 12.80 (s, 1H), 10.84 (s, 1H), 8.76 (s, 1H), 8.17 (d, J=2.0 Hz, 1H), 8.13 (s, 1H), 8.13 (s, 1H), 8.02 (s, 1H), 7.73 (dd, J=9.2, 4.4 Hz, 1H), 7.49-7.43 (m, 2H), 7.38-7.20 (m, 2H), 6.88 (d, J=8.8 Hz, 1H), 6.80 (s, 1H), 6.18 (bs, 1H), 4.46-4.37 (m, 5H), 4.35-4.23 (m, 1H), 3.88 (s, 3H), 3.56 (s, 3H), 3.39-3.37 (m, 1H), 3.17 (bs, 1H), 2.94 (bs, 2H), 2.77-2.76 (m, 1H), 2.68-2.58 (m, 2H), 2.23-2.10 (m, 2H), 2.17-2.14 (m, 1H). 2.07-1.90 (s, 1H), 1.70-1.40 (m, 2H), 1.41-1.30 (s, 1H), 1.25-1.17 (m, 6H), 0.80-0.71 (m, 1H), 0.61-0.50 (m, 2H), 0.40-0.32 (m, 1H).

LC-MS (ESI): m/z=884.50 [M+H]⁺

### Example 156: 3-(7-((3R, 5S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,5-dimethylpiperazin-1-yl)-1-methyl-1H-indazol-3-yl)piperidine-2,6-dione (Compound 506a)

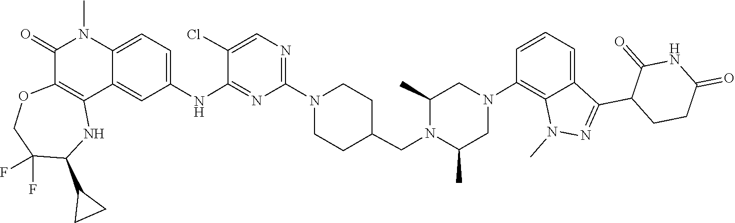 [SMILES: C[C@@H]1CN(c2cccc3c(C4CCC(=O)NC4=O)nn(C)c23)C[C@H](C)N1CC1CCN(c2ncc(Cl)c(Nc3ccc4c(c3)c3c(c(=O)n4C)OCC(F)(F)[C@H](C4CC4)N3)n2)CC1]

### Preparation of tert-butyl (3S, 5R)-4-((1-((benzyloxy)carbonyl) piperidin-4-yl)methyl)-3,5-dimethylpiperazine-1-carboxylate

To a stirred solution of tert-butyl (3S, 5R)-3,5-dimethylpiperazine-1-carboxylate (3.0 g, 14.00 mmol) and benzyl 4-formylpiperidine-1-carboxylate (4.15 g, 16.80 mmol) in DCM (60 mL) was added acetic acid (0.80 mL, 14.00 mmol) at 0° C. and stirred for 2 h. To this solution was added NaBH(OAc)₃ (5.93 g, 18.50) at 0° C. The mixture was stirred at room temperature for 16 h. The reaction mixture was quenched with ice-cold water (100 mL) and extracted with DCM (3×100 mL). The combined organic layers were washed with brine (100 mL), dried over anhydrous Na₂SO₄, filtered, and dried under vacuum to get a crude product which was purified by column chromatography (25% ethyl acetate in petroleum ether) to afford tert-butyl (3S, 5R)-4-((1-((benzyloxy)carbonyl) piperidin-4-yl)methyl)-3,5-dimethylpiperazine-1-carboxylate (1.8 g) as a colorless liquid.

LC-MS (ESI): m/z=446.61 [M+H]⁺

### Preparation of benzyl 4-(((2S, 6R)-2,6-dimethylpiperazin-1-yl)methyl)piperidine-1-carboxylate

To a stirred solution of tert-butyl (3S, 5R)-4-((1-((benzyloxy)carbonyl) piperidin-4-yl)methyl)-3,5-dimethylpiperazine-1-carboxylate (1.8 g, 4.04 mmol) in THF (54 mL) was added TFA (18 mL). The mixture was stirred at room temperature for 16 h. The reaction mixture was filtered through a celite bed, concentrated under reduced pressure, washed with n-pentane, and dried under vacuum to afford benzyl 4-(((2S, 6R)-2,6-dimethylpiperazin-1-yl)methyl)piperidine-1-carboxylate (1.2 g) as a brown solid which was used in the next step without further purification.

LC-MS (ESI): m/z=346.43 [M+H]⁺

### Preparation of benzyl 4-(((2R, 6S)-4-(3-(2,6-bis(benzyloxy)pyridin-3-yl)-1-methyl-1H-indazol-7-yl)-2,6-dimethylpiperazin-1-yl)methyl)piperidine-1-carboxylate

A stirred solution of 3-(2,6-bis(benzyloxy)pyridin-3-yl)-7-bromo-1-methyl-1H-indazole (0.80 g, 1.60 mmol), benzyl 4-(((2S, 6R)-2,6-dimethylpiperazin-1-yl)methyl)piperidine-1-carboxylate (1.12 g, 3.20 mmol) and cesium carbonate (1.5 g, 4.8 mmol) in 1,4-dioxane (16 mL) was degassed for 10 minutes with nitrogen gas. To this mixture was added Pd PEPSI iHeptCl (0.07 g, 0.08 mmol). The mixture was stirred at 100° C. for 12 h. The reaction mixture was cooled to room temperature, filtered through a celite bed, and concentrated under reduced pressure to get a crude product which was purified by flash column chromatography (25% ethyl acetate in petroleum ether) to afford benzyl 4-(((2R, 6S)-4-(3-(2,6-bis(benzyloxy)pyridin-3-yl)-1-methyl-1H-indazol-7-yl)-2,6-dimethylpiperazin-1-yl)methyl)piperidine-1-carboxylate as a brown solid (0.7 g).

LC-MS (ESI): m/z=765.76 [M+H]⁺

### Preparation of 3-(7-((3R, 5S)-3,5-dimethyl-4-(piperidin-4-ylmethyl)piperazin-1-yl)-1-methyl-1H-indazol-3-yl)piperidine-2,6-dione

To a stirred solution of benzyl 4-(((2R, 6S)-4-(3-(2,6-bis(benzyloxy)pyridin-3-yl)-1-methyl-1H-indazol-7-yl)-2,6-dimethylpiperazin-1-yl)methyl)piperidine-1-carboxylate (0.70 g, 0.91 mmol) in THF (28 mL) and AcOH (0.70 mL) was added 20% Pd(OH)₂ (0.70 g, 100% w/w). The mixture was stirred under hydrogen atmosphere (70 psi) at room temperature for 16 h. The reaction mixture was diluted with THF and filtered through a pad of celite and washed with excess of 20% THF in DCM. The filtrate was collected and concentrated to afford 3-(7-((3R, 5S)-3,5-dimethyl-4-(piperidin-4-ylmethyl)piperazin-1-yl)-1-methyl-1H-indazol-3-yl)piperidine-2,6-dione as a brown solid (0.22 g) which was used in the next step without further purification.

LC-MS (ESI): m/z=453.48 [M+H]⁺

### Preparation of 3-(7-((3R, 5S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,5-dimethylpiperazin-1-yl)-1-methyl-1H-indazol-3-yl)piperidine-2,6-dione

To a stirred solution of 3-(7-((3R, 5S)-3,5-dimethyl-4-(piperidin-4-ylmethyl)piperazin-1-yl)-1-methyl-1H-indazol-3-yl)piperidine-2,6-dione (0.11 g, 0.24 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.09 mmol)) in DMSO (3.0 mL) was added DIPEA (0.24 mL, 1.92 mmol). The mixture was heated at 100° C. and stirred for 12 h. The reaction mixture was quenched with ice-cold water. The solid was filtered to get a crude product which was purified by prep-HPLC to afford 3-(7-((3R, 5S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,5-dimethylpiperazin-1-yl)-1-methyl-1H-indazol-3-yl)piperidine-2,6-dione as an off-white solid (0.053 g).

¹H NMR 400 MHz, DMSO-d6: δ 10.87 (s, 1H), 8.76 (s, 1H), 8.28 (s, 1H), 8.17 (d, J=1.6 Hz, 1H), 8.05 (s, 1H), 7.74 (dd, J=7.6, 1.60 Hz, 1H), 7.44 (d, J=8.80 Hz, 1H), 7.38 (d, J=7.60 Hz, 1H), 6.97-6.98 (m, 2H), 6.19 (s, 1H), 4.50-4.33 (m, 5H), 4.23 (s, 1H), 3.57 (s, 3H), 3.32 (s, 4H), 3.14 (d, J=11.20 Hz, 2H), 2.61-2.62 (m, 4H), 2.59-0.00 (m, 2H), 2.50-2.50 (m, 2H), 2.38 (s, 1H), 2.30-2.31 (m, 1H), 1.77 (s, 2H), 1.60 (s, 1H), 1.42 (s 1H), 1.10-0.90 (m, 8H), 0.80-0.70 (m, 1H), 0.60-0.52 (m, 2H), 0.40-0.36 (m, 1H).

LC-MS (ESI): m/z=884.48 [M+H]⁺

### Example 157: 3-(7-((3S, 5S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,5-dimethylpiperazin-1-yl)-1-methyl-1H-indazol-3-yl)piperidine-2,6-dione (Compound 506b)

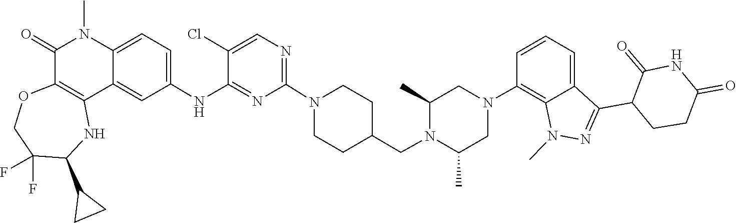 [SMILES: C[C@H]1CN(c2cccc3c(C4CCC(=O)NC4=O)nn(C)c23)C[C@H](C)N1CC1CCN(c2ncc(Cl)c(Nc3ccc4c(c3)c3c(c(=O)n4C)OCC(F)(F)[C@H](C4CC4)N3)n2)CC1]

### Preparation of tert-butyl (3S, 5S)-4-((1-((benzyloxy)carbonyl) piperidin-4-yl)methyl)-3,5-dimethylpiperazine-1-carboxylate

To a stirred solution of tert-butyl (3S, 5S)-3,5-dimethylpiperazine-1-carboxylate (3.0 g, 14.00 mmol) and benzyl 4-formylpiperidine-1-carboxylate (4.15 g, 16.80 mmol) in THF (60 mL), acetic acid (0.84 g, 14.00 mmol) was added at 0° C. The reaction mixture was stirred for 2 h.

NaBH(OAc)₃ (5.93 g, 18.50) was added at 0° C. stirred reaction mixture at room temperature for 14 h. The reaction mixture was quenched with ice-cold water (100 mL) and extracted with DCM (3×100 mL). The combined organic layers were washed with brine (100 mL), dried over anhydrous Na₂SO₄, filtered, and concentrated under vacuum to get a crude product which was purified by column chromatography (25% ethyl acetate in petroleum ether) to afford tert-butyl (3S, 5S)-4-((1-((benzyloxy)carbonyl) piperidin-4-yl)methyl)-3,5-dimethylpiperazine-1-carboxylate (1.8 g) as a yellow liquid.

LC-MS (ESI): m/z=446.56 [M+H]⁺

### Preparation of benzyl 4-(((2S, 6S)-2,6-dimethylpiperazin-1-yl)methyl)piperidine-1-carboxylate

To a stirred solution of tert-butyl (3S, 5S)-4-((1-((benzyloxy)carbonyl) piperidin-4-yl)methyl)-3,5-dimethylpiperazine-1-carboxylate (1.8 g, 4.04 mmol) in DCM (36 mL) was added TFA (18 mL).

The mixture was stirred at room temperature for 16 h. The reaction mixture was concentrated under reduced pressure to get the crude product which was triturated with n-pentane and dried under vacuum to afford benzyl 4-(((2S, 6S)-2,6-dimethylpiperazin-1-yl)methyl)piperidine-1-carboxylate (1.2 g) as a brown solid.

LC-MS (ESI): m/z=346.23 [M+H]⁺

### Preparation of benzyl 4-(((2S, 6S)-4-(3-(2,6-bis(benzyloxy)pyridin-3-yl)-1-methyl-1H-indazol-7-yl)-2,6-dimethylpiperazin-1-yl)methyl)piperidine-1-carboxylate

A stirred solution of 3-(2,6-bis(benzyloxy)pyridin-3-yl)-7-bromo-1-methyl-1H-indazole (0.80 g, 1.60 mmol), benzyl 4-(((2S, 6S)-2,6-dimethylpiperazin-1-yl)methyl)piperidine-1-carboxylate (1.12 g, 3.20 mmol) and cesium carbonate (1.5 g, 4.8 mmol) in 1,4-dioxane (16 mL) was degassed for 10 minutes. To this mixture was added Pd PEPPSI iHeptCl (0.07 g, 0.08 mmol). The mixture was stirred at 100° C. for 12 h. The reaction mixture was cooled to room temperature, filtered through a celite bed, and concentrated under reduced pressure to get a crude product which was purified by flash column chromatography (25% ethyl acetate in petroleum ether) to afford benzyl 4-(((2S, 6S)-4-(3-(2,6-bis(benzyloxy)pyridin-3-yl)-1-methyl-1H-indazol-7-yl)-2,6-dimethylpiperazin-1-yl)methyl)piperidine-1-carboxylate as a brown solid (0.7 g).

LC-MS (ESI): m/z=765.75 [M+H]⁺

### Preparation of 3-(7-((3S, 5S)-3,5-dimethyl-4-(piperidin-4-ylmethyl)piperazin-1-yl)-1-methyl-1H-indazol-3-yl)piperidine-2,6-dione

To a stirred solution of benzyl 4-(((2S, 6S)-4-(3-(2,6-bis(benzyloxy)pyridin-3-yl)-1-methyl-1H-indazol-7-yl)-2,6-dimethylpiperazin-1-yl)methyl)piperidine-1-carboxylate (0.70 g, 0.91 mmol) in THF (28 mL) were added AcOH (0.70 mL) and 20% Pd(OH)₂ on carbon (0.70 g). The mixture was stirred under hydrogen atmosphere (80 psi) at room temperature for 16 h. The reaction mixture was diluted with THF, filtered through a pad of celite, and washed with excess of 20% THF in DCM. The filtrate was collected and concentrated to afford 3-(7-((3S, 5S)-3,5-dimethyl-4-(piperidin-4-ylmethyl)piperazin-1-yl)-1-methyl-1H-indazol-3-yl)piperidine-2,6-dione as a brown solid (0.22 g) which was used in the next step without further purification.

LC-MS (ESI): m/z=453.47 [M+H]⁺

### Preparation of 3-(7-((3S, 5S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,5-dimethylpiperazin-1-yl)-1-methyl-1H-indazol-3-yl)piperidine-2,6-dione

To a stirred solution of 3-(7-((3S, 5S)-3,5-dimethyl-4-(piperidin-4-ylmethyl)piperazin-1-yl)-1-methyl-1H-indazol-3-yl)piperidine-2,6-dione (0.11 g, 0.24 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.09 mmol)) in DMSO (3.0 mL) was added DIPEA (0.24 mL, 1.92 mmol). The mixture was heated at 100° C. and stirred for 12 h. The reaction mixture was quenched with ice-cold water, and the solid was filtered to get crude product which was purified by prep-HPLC to afford 3-(7-((3S, 5S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,5-dimethylpiperazin-1-yl)-1-methyl-1H-indazol-3-yl)piperidine-2,6-dione as an off-white solid (0.039 g).

¹H NMR 400 MHz, DMSO-d6: δ 10.88 (s, 1H), 8.77 (s, 1H), 8.18 (d, J=8.40 Hz, 2H), 8.03 (s, 1H), 7.74 (d, J=7.60 Hz, 1H), 7.45 (d, J=8.80 Hz, 1H), 7.39 (d, J=7.20 Hz, 1H), 7.10-7.00 (m, 2H), 6.19 (s, 1H), 4.45-4.43 (m, 5H), 4.26 (s, 3H), 3.57 (s, 3H), 3.16-3.14 (m, 6H), 2.80 (q, J=Hz, 2H), 2.58 (q, J=1.60 Hz, 2H), 2.17 (m, 4H), 1.78-1.70 (m, 3H), 0.98-0.95 (m, 9H), 0.78-070 (m, 1H), 0.53-0.50 (m, 2H), 0.37-0.34 (m, 1H).

LC-MS (ESI): m/z=884.45 [M+H]⁺

### Example 158: 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-7-fluoro-1-methyl-1H-indazol-3-yl)piperidine-2,6-dione (Compound 507a)

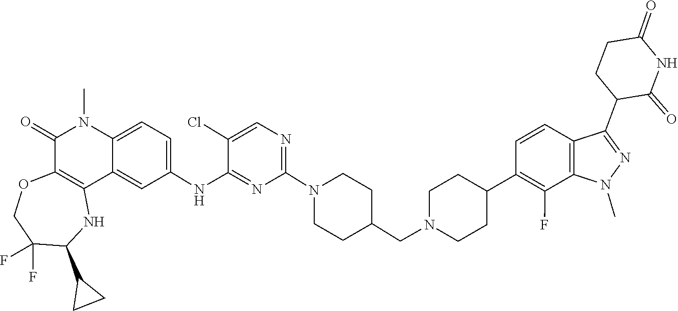 [SMILES: Cn1nc(C2CCC(=O)NC2=O)c2ccc(C3CCN(CC4CCN(c5ncc(Cl)c(Nc6ccc7c(c6)c6c(c(=O)n7C)OCC(F)(F)[C@H](C7CC7)N6)n5)CC4)CC3)c(F)c21]

### Preparation of tert-butyl 4-(3-(2,6-bis(benzyloxy)pyridin-3-yl)-7-fluoro-1-methyl-1H-indazol-6-yl)-3,6-dihydropyridine-1(2H)-carboxylate

To a stirred solution of 3-(2,6-bis(benzyloxy)pyridin-3-yl)-6-bromo-7-fluoro-1-methyl-1H-indazole (0.30 g, 0.58 mmol) and tert-butyl 4-(4,4,5,5-tetramethyl-1,3,2-dioxaborolan-2-yl)-3,6-dihydropyridine-1(2H)-carboxylate (0.23 g, 0.75 mmol) in dioxane (3.0 mL) and water (0.60 mL), was added cesium carbonate (0.566 g, 1.74 mmol). The mixture was purged with N₂ for 15 min followed by the addition of Pd₂(dba)₃ (0.03 g, 0.03 mmol) and xantPhos (0.033 g, 0.06 mmol).

The mixture was heated at 100° C. and stirred for 6 h. The reaction mixture was cooled to room temperature, diluted with ethyl acetate (300 mL), filtered through a celite bed and washed with DCM (200 mL). The filtrate was concentrated to get a crude product, which was purified by flash chromatography (5-10% of ethyl acetate in petroleum ether) to afford tert-butyl 4-(3-(2,6-bis(benzyloxy)pyridin-3-yl)-7-fluoro-1-methyl-1H-indazol-6-yl)-3,6-dihydropyridine-1(2H)-carboxylate as a pale-yellow semisolid (0.30 g).

LC-MS (ESI): m/z=621.60 [M+H]⁺

### Preparation of 3-(2,6-bis(benzyloxy)pyridin-3-yl)-7-fluoro-1-methyl-6-(1,2,3,6-tetrahydropyridin-4-yl)-1H-indazole and 6-(benzyloxy)-5-(7-fluoro-1-methyl-6-(1,2,3,6-tetrahydropyridin-4-yl)-1H-indazol-3-yl)pyridin-2-ol

To a stirred solution of tert-butyl 4-(3-(2,6-bis(benzyloxy)pyridin-3-yl)-7-fluoro-1-methyl-1H-indazol-6-yl)-3,6-dihydropyridine-1(2H)-carboxylate (0.30 g, 0.48 mmol) in DCM (6 mL) at 0° C. was added TFA (2.40 mL). The mixture was stirred at room temperature for 2 h. The reaction mixture was concentrated under reduced pressure to afford a crude mixture of 3-(2,6-bis(benzyloxy)pyridin-3-yl)-7-fluoro-1-methyl-6-(1,2,3,6-tetrahydropyridin-4-yl)-1H-indazole and 6-(benzyloxy)-5-(7-fluoro-1-methyl-6-(1,2,3,6-tetrahydropyridin-4-yl)-1H-indazol-3-yl)pyridin-2-ol (0.33 g) as a pale brown semi solid. The crude mixture was taken forward to the next step without further purification.

LC-MS (ESI): m/z=521.50 [M+H]⁺; m/z=431.45 [M+H]⁺

### Preparation of tert-butyl 4-((4-(3-(2,6-bis(benzyloxy)pyridin-3-yl)-7-fluoro-1-methyl-1H-indazol-6-yl)-3,6-dihydropyridin-1(2H)-yl)methyl)piperidine-1-carboxylate and tert-butyl 4-((4-(3-(2-(benzyloxy)-6-hydroxypyridin-3-yl)-7-fluoro-1-methyl-1H-indazol-6-yl)-3,6-dihydropyridin-1(2H)-yl)methyl)piperidine-1-carboxylate

To a stirred solution of a 3-(2,6-bis(benzyloxy)pyridin-3-yl)-7-fluoro-1-methyl-6-(1,2,3,6-tetrahydropyridin-4-yl)-1H-indazole and 6-(benzyloxy)-5-(7-fluoro-1-methyl-6-(1,2,3,6-tetrahydropyridin-4-yl)-1H-indazol-3-yl)pyridin-2-ol mixture (0.33 g, 0.63) and tert-butyl 4-formylpiperidine-1-carboxylate (0.15 g, 0.70 mmol) in THF (16 mL) were added sodium acetate (0.16 g, 1.90 mmol) and acetic acid (0.33 mL). The mixture was stirred at room temperature for 1 h followed by the addition of sodium triacetoxyborohydride (0.40 g, 1.90 mmol) at 0° C. The mixture was stirred at room temperature for 16 h. The reaction mixture was quenched with water (50 mL) and extracted with ethyl acetate (100 mL). The combined organic layers were washed with brine, dried over anhydrous Na₂SO₄, filtered, and concentrated under vacuum to obtain crude product which was triturated with n-pentane to afford a crude mixture of tert-butyl 4-((4-(3-(2,6-bis(benzyloxy)pyridin-3-yl)-7-fluoro-1-methyl-1H-indazol-6-yl)-3,6-dihydropyridin-1(2H)-yl)methyl)piperidine-1-carboxylate and tert-butyl 4-((4-(3-(2-(benzyloxy)-6-hydroxypyridin-3-yl)-7-fluoro-1-methyl-1H-indazol-6-yl)-3,6-dihydropyridin-1(2H)-yl)methyl)piperidine-1-carboxylate (0.48 g) as a pale yellow semi-solid.

LC-MS (ESI): m/z=628.61 [M+H]⁺; m/z=718.76 [M+H]⁺

### Preparation of tert-butyl 4-((4-(3-(2,6-dioxopiperidin-3-yl)-7-fluoro-1-methyl-1H-indazol-6-yl)piperidin-1-yl)methyl)piperidine-1-carboxylate

To a stirred solution of a tert-butyl 4-((4-(3-(2,6-bis(benzyloxy)pyridin-3-yl)-7-fluoro-1-methyl-1H-indazol-6-yl)-3,6-dihydropyridin-1(2H)-yl)methyl)piperidine-1-carboxylate and tert-butyl 4-((4-(3-(2-(benzyloxy)-6-hydroxypyridin-3-yl)-7-fluoro-1-methyl-1H-indazol-6-yl)-3,6-dihydropyridin-1(2H)-yl)methyl)piperidine-1-carboxylate mixture (0.48 g, 0.67 mmol) in THF (81 mL) was added 20% Pd(OH)₂ (0.48 g). The mixture was stirred under hydrogen atmosphere (80 psi) at room temperature for 18 h. The reaction mixture was diluted with DCM (200 mL), washed with 30% THF:DCM (300 mL) and filtered through a celite bed. The collected filtrate was concentrated under vacuum to obtain crude product which was triturated with n-pentane to afford tert-butyl 4-((4-(3-(2,6-dioxopiperidin-3-yl)-7-fluoro-1-methyl-1H-indazol-6-yl)piperidin-1-yl)methyl)piperidine-1-carboxylate (0.29 g) as an off-white solid.

LC-MS (ESI): m/z=542.84 [M+H]⁺

### Preparation of 3-(7-fluoro-1-methyl-6-(1-(piperidin-4-ylmethyl)piperidin-4-yl)-1H-indazol-3-yl)piperidine-2,6-dione

To a stirred solution of tert-butyl 4-((4-(3-(2,6-dioxopiperidin-3-yl)-7-fluoro-1-methyl-1H-indazol-6-yl)piperidin-1-yl)methyl)piperidine-1-carboxylate (0.290 g, 0.535 mmol) in DCM (3 mL) at 0° C. was added TFA (2.3 mL). The mixture was stirred at room temperature for 2 h. The reaction mixture was concentrated under reduced pressure to afford 3-(7-fluoro-1-methyl-6-(1-(piperidin-4-ylmethyl)piperidin-4-yl)-1H-indazol-3-yl)piperidine-2,6-dione (0.32 g) as a pale brown semi-solid which was used in the next step without further purification.

LC-MS (ESI): m/z=442.36 [M+H]⁺

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-7-fluoro-1-methyl-1H-indazol-3-yl)piperidine-2,6-dione

To a stirred solution of 3-(7-fluoro-1-methyl-6-(1-(piperidin-4-ylmethyl)piperidin-4-yl)-1H-indazol-3-yl)piperidine-2,6-dione (0.32 g, 0.73 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.10 g, 0.22 mmol) in DMSO (3 mL) was added DIPEA (3.20 mL). The mixture was heated at 100° C. and stirred for 18 h. The reaction mixture was quenched with ice cold water (100 mL), and the precipitated solid was filtered and dried under vacuum. The crude product was purified by prep-HPLC to afford 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-7-fluoro-1-methyl-1H-indazol-3-yl)piperidine-2,6-dione (0.06 g) as an off-white solid. ¹H NMR: (400 MHz, DMSO-d6): δ 10.92 (s, 1H), 8.89 (br s, 2H), 8.07 (s, 1H), 7.75-7.73 (m, 1H), 7.53 (d, J=8.40 Hz, 1H), 7.43 (d, J=8.80 Hz, 1H), 7.21-6.95 (m, 1H), 6.24 (s, 1H), 4.46-4.34 (m, 5H), 4.12 (s, 3H), 3.57-3.53 (m, 4H), 3.32-3.23 (m, 2H), 3.15-3.12 (m, 2H), 2.87-2.82 (m, 2H), 2.62-2.58 (m, 2H), 2.40-2.38 (m, 1H), 2.18-2.07 (m, 4H), 1.97-1.94 (m, 2H), 1.75-1.73 (m, 2H), 1.35-1.34 (m, 4H), 1.14-1.2 (m, 2H), 0.72-0.71 (m, 1H), 0.52-0.49 (m, 2H), 0.36-0.34 (m, 1H).

LC-MS (ESI): m/z=873.46 [M+H]⁺

### Example 159: 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,3-dimethylpiperazin-1-yl)-1-methyl-1H-indazol-3-yl)piperidine-2,6-dione (Compound 510a)

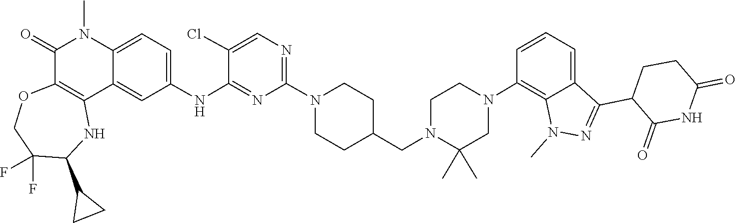 [SMILES: Cn1nc(C2CCC(=O)NC2=O)c2cccc(N3CCN(CC4CCN(c5ncc(Cl)c(Nc6ccc7c(c6)c6c(c(=O)n7C)OCC(F)(F)[C@H](C7CC7)N6)n5)CC4)C(C)(C)C3)c21]

### Preparation of benzyl 4-((4-(3-(2,6-bis(benzyloxy)pyridin-3-yl)-1-methyl-1H-indazol-7-yl)-2,2-dimethylpiperazin-1-yl)methyl)piperidine-1-carboxylate

A stirred solution of benzyl 4-((2,2-dimethylpiperazin-1-yl)methyl)piperidine-1-carboxylate (2.0 g, 5.78 mmol), 3-(2,6-bis(benzyloxy)pyridin-3-yl)-7-bromo-1-methyl-1H-indazole (1.44 g, 2.894 mmol), and sodium tert-butoxide solution in THF (8.68 mL, 7.367 mmol) in 1,4-dioxane (20 mL) was purged with N₂ for 5 min. To this mixture was added Ruphos-Pd-G3 (0.485 g, 0.579 mmol).

The mixture was heated at 100° C. and stirred for 16 h. The reaction mixture was quenched with ice cold water (20 mL) and extracted with ethyl acetate (100 mL). The combined organic layer was washed with brine (50 mL), dried over anhydrous sodium sulfate, and filtered and concentrated to get crude compound which was purified by flash column chromatography (50-60% ethyl acetate in petroleum ether) to afford benzyl 4-((4-(3-(2,6-bis(benzyloxy)pyridin-3-yl)-1-methyl-1H-indazol-7-yl)-2,2-dimethylpiperazin-1-yl)methyl)piperidine-1-carboxylate (0.70 g).

LC-MS (ESI): m/z=765.68 [M+H]⁺

### Preparation of 3-(7-(3,3-dimethyl-4-(piperidin-4-ylmethyl)piperazin-1-yl)-1-methyl-1H-indazol-3-yl)piperidine-2,6-dione

A stirred solution of benzyl 4-((4-(3-(2,6-bis(benzyloxy)pyridin-3-yl)-1-methyl-1H-indazol-7-yl)-2,2-dimethylpiperazin-1-yl)methyl)piperidine-1-carboxylate (0.50 g, 0.65 mmol) in THF (15 mL), was added 20% Pd(OH)₂ on carbon (0.50 g, 100% w/w). The mixture was stirred with hydrogen pressure (80 Psi) at room temperature for 16 h. The reaction mixture was diluted with ethyl acetate (50 mL) and filtered through a celite bed. The filtrate was collected and concentrated to get a crude product which was purified by trituration with diethyl ether to afford 3-(7-(3,3-dimethyl-4-(piperidin-4-ylmethyl)piperazin-1-yl)-1-methyl-1H-indazol-3-yl)piperidine-2,6-dione (0.20 g).

LC-MS (ESI): m/z=453.47 [M+H]⁺

### Preparation of 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,3-dimethylpiperazin-1-yl)-1-methyl-1H-indazol-3-yl)piperidine-2,6-dione

To a stirred solution of 3-(7-(3,3-dimethyl-4-(piperidin-4-ylmethyl)piperazin-1-yl)-1-methyl-1H-indazol-3-yl)piperidine-2,6-dione (0.100 g, 0.22 mmol) and (S)-2-cyclo propyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.052 g, 0.11 mmol) in DMSO (2 mL) was added DIPEA (0.118 mL, 0.663 mmol) at room temperature. The mixture was heated at 100° C. and stirred for 3 h. The reaction mixture was concentrated under reduced pressure and purified by prep-HPLC to afford 3-(7-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3,3-dimethylpiperazin-1-yl)-1-methyl-1H-indazol-3-yl)piperidine-2,6-dione (0.025 g) as an off white solid.

¹H NMR (400 MHz, DMSO-d6): 10.87 (s, 1H), 8.76 (s, 1H), 8.18 (s, 1H), 8.02 (s, 1H), 7.73 (d, J=8.8 Hz, 1H), 7.45-7.38 (m, 2H), 7.04-7.00 (m, 2H), 6.18 (s, 1H), 4.50-4.20 (m, 8H), 3.60 (s, 3H). 3.31-3.25 (m, 2H) 2.77-260 (m, 8H), 2.58-2.50 (m, 2H), 2.37 (bs, 1H), 2.18-2.13 (m, 1H), 1.94-1.90 (m, 1H), 1.62 (br s, 1H), 1.33 (bs, 1H), 1.23 (s, 1H), 1.07 (bs, 7H), 0.95-0.70 (m, 1H), 0.65-0.48 (m, 2H), 0.40-0.35 (m, 1H).

LC-MS (ESI): m/z=884.48 [M+H]⁺

### Example 160: 3-(6-(1-((S)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione

### [3-(6-(1-((S*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione](Compound 522a)

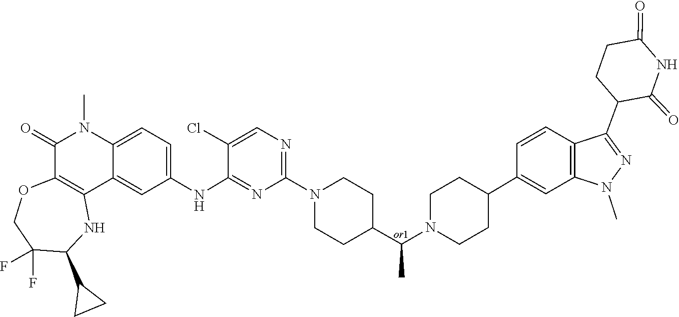 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCC(c2ccc3c(C4CCC(=O)NC4=O)nn(C)c3c2)CC1]

### Preparation of tert-butyl 4-(3-(2,6-bis(benzyloxy)pyridin-3-yl)-1-methyl-1H-indazol-6-yl)-3,6-dihydropyridine-1(2H)-carboxylate

A solution of 3-(2,6-bis(benzyloxy)pyridin-3-yl)-6-bromo-1-methyl-1H-indazole (5.00 g 9.99 mmol), tert-butyl 4-(4,4,5,5-tetramethyl-1,3,2-dioxaborolan-2-yl)-3,6-dihydropyridine-1(2H)-carboxylate (3.70 g, 11.98 mmol), and cesium carbonate (9.76 g, 29.97 mmol) in 1,4-dioxane (200 mL) and water (100 mL) was degassed with argon for 10 minutes. To this mixture were added Pd₂(dba)₃ (0.45 g, 0.49 mmol) and XantPhos (0.57 g, 0.99 mmol). The mixture was heated at 100° C. and stirred for 18 h. The reaction mixture was diluted with DCM (100 mL) and filtered through a celite bed. The collected filtrate was concentrated under reduced pressure to obtain a crude product which was purified by flash column chromatography (90% ethyl acetate in petroleum ether) to afford tert-butyl 4-(3-(2,6-bis(benzyloxy)pyridin-3-yl)-1-methyl-1H-indazol-6-yl)-3,6-dihydropyridine-1(2H)-carboxylate (5.0 g) as a pale-yellow solid.

LC-MS (ESI): m/z=603.54 [M+H]⁺

### Preparation of 3-(2,6-bis(benzyloxy)pyridin-3-yl)-1-methyl-6-(1,2,3,6-tetrahydropyridin-4-yl)-1H-indazole

To a stirred solution of tert-butyl 4-(3-(2,6-bis(benzyloxy)pyridin-3-yl)-1-methyl-1H-indazol-6-yl)-3,6-dihydropyridine-1(2H)-carboxylate (5.0 g, 8.29 mmol)) in DCM (150 mL) cooled to 0° C. was added 4M HCl in dioxane (25 mL). The mixture was stirred at room temperature for 2 h. The reaction mixture was concentrated under reduced pressure and co-distilled with diethyl ether to get a crude product. The crude product was washed with n-pentane to obtain 3-(2,6-bis(benzyloxy)pyridin-3-yl)-1-methyl-6-(1,2,3,6-tetrahydropyridin-4-yl)-1H-indazole as a semi-solid.

LC-MS (ESI): m/z=503.57 [M+H]⁺

### Preparation of tert-butyl 4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate

To a stirred solution of 3-(2,6-bis(benzyloxy)pyridin-3-yl)-1-methyl-6-(1,2,3,6-tetrahydropyridin-4-yl)-1H-indazole (4.00 g, 7.95 mmol) and tert-butyl 4-acetylpiperidine-1-carboxylate (3.61 g, 15.91 mmol) in ethanol (80 mL) was added titanium(IV) isopropoxide (4.0 mL) and 4A° molecular sieves (2.0 g, 50% w/w). The mixture was stirred at 70° C. for 48 h. To this mixture was added sodium cyanoborohydride (1.49 g, 23.85 mmol) portionwise at 0° C. The mixture was stirred at 70° C. for 16 h. The reaction mixture was quenched with ice-cold water (100 mL) and extracted with DCM (3×100 mL). The combined organic layer was washed with brine (100 mL), dried over anhydrous Na₂SO₄, filtered, and dried under vacuum to get a crude product, which was purified by flash column chromatography (10% methanol in DCM) to afford tert-butyl 4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate (1.0 g) as a brown semi-solid.

LC-MS (ESI): m/z=714.60 [M+H]⁺

### Preparation of tert-butyl (S)-4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate and tert-butyl (R)-4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate

Stereoisomers of tert-butyl 4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate were separated by SFC to afford tert-butyl (S)-4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate (0.170 g) and tert-butyl (R)-4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate (0.650 g) as semi-solids.

### Preparation of tert-butyl 4-((1S)-1-(4-(3-(2,6-dioxopiperidin-3-yl)-1-methyl-1H-indazol-6-yl)piperidin-1-yl)ethyl)piperidine-1-carboxylate

To a stirred solution of tert-butyl (S)-4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate (0.17 g, 0.23 mmol) in THF (6.8 mL) was added 20% Pd(OH)₂ (0.170 g, 100% w/w). The mixture was stirred under hydrogen atmosphere (80 psi) for 12 h at room temperature. The reaction mixture was diluted with THF, filtered through a pad of celite, and washed with an excess of 20% THF in DCM(50 mL).

The filtrate was collected and concentrated to afford tert-butyl 4-((1S)-1-(4-(3-(2,6-dioxopiperidin-3-yl)-1-methyl-1H-indazol-6-yl)piperidin-1-yl)ethyl)piperidine-1-carboxylate (0.150 g) which was directly used in the next step without purification.

LC-MS (ESI): m/z=538.52 [M+H]⁺

### Preparation of 3-(1-methyl-6-(1-((S)-1-(piperidin-4-yl)ethyl)piperidin-4-yl)-1H-indazol-3-yl)piperidine-2,6-dione

To a stirred solution of tert-butyl 4-((1S)-1-(4-(3-(2,6-dioxopiperidin-3-yl)-1-methyl-1H-indazol-6-yl)piperidin-1-yl)ethyl)piperidine-1-carboxylate (1.0 g, 1.62 mmol)) in DCM (20.0 mL) cooled to 0° C. was added TFA (5.0 mL). The mixture was stirred at room temperature for 2 h. The reaction mixture was concentrated under reduced pressure and co-distilled with diethyl ether to get crude product which was washed with n-pentane to obtain 3-(1-methyl-6-(1-((S)-1-(piperidin-4-yl)ethyl)piperidin-4-yl)-1H-indazol-3-yl)piperidine-2,6-dione (0.150 g) as a semi-solid.

LC-MS (ESI): m/z=438.51 [M+H]⁺

### Preparation of 3-(6-(1-((S)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione [3-(6-(1-((S*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione]

To a stirred solution of 3-(1-methyl-6-(1-((S)-1-(piperidin-4-yl)ethyl)piperidin-4-yl)-1H-indazol-3-yl)piperidine-2,6-dione (0.150 g, 0.34 mmol) in DMSO (3.0 mL) were added N,N-diisopropylethylamine (0.21 g, 1.72 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.64 g, 0.13 mmol). The mixture was stirred at 100° C. for 6 h. The reaction mixture was quenched with ice-cold water (20 mL) and stirred for 5 min. The precipitated solid was filtered, dried under vacuum, and purified by prep-HPLC to afford 3-(6-(1-((S)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione [3-(6-(1-((S*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione](6.8 mg) as an off white solid.

LC-MS (ESI): m/z=869.51 [M+H]⁺

¹H NMR (400 MHz, DMSO-d₆): δ 10.89 (s, 1H), 8.91 (s, 1H), 8.19 (s, 1H), 8.06 (s, 1H), 7.72-7.67 (m, 2H), 7.43 (d, J=8.80 Hz, 1H), 7.38 (s, 1H), 7.21-6.95 (m, 2H), 6.24 (s, 1H), 4.53-4.32 (m, 5H), 3.99 (s, 3H), 3.633.56 (m, 5H), 3.23-3.01 (m, 5H), 2.82-2.79 (m, 1H), 2.82-2.76 (m, 2H), 2.62-2.59 (m, 2H), 2.38-2.36 (m, 1H), 2.18-2.14 (m, 4H), 1.71-1.62 (m, 2H), 1.31-1.17 (m, 6H), 0.73-0.71 (m, 1H), 0.53-0.50 (m, 2H), 0.36-0.34 (m, 1H).

### Example 161: 3-(6-(1-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione

### [3-(6-(1-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione](Compound 522b)

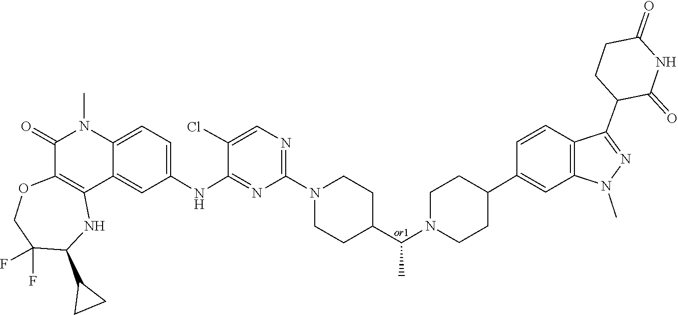 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCC(c2ccc3c(C4CCC(=O)NC4=O)nn(C)c3c2)CC1]

### Preparation of tert-butyl 4-((1R)-1-(4-(3-(2,6-dioxopiperidin-3-yl)-1-methyl-1H-indazol-6-yl)piperidin-1-yl)ethyl)piperidine-1-carboxylate

To a stirred solution of tert-butyl (R)-4-(1-(4-(3-(2,6-bis(benzyloxy)pyridin-3-yl)-1-methyl-1H-indazol-6-yl)-3,6-dihydropyridin-1(2H)-yl)ethyl)piperidine-1-carboxylate (1.0 g, 1.51 mmol) in THF (13.0 mL) was added 20% Pd(OH)₂ (0.6 g, 100% w/w). The mixture was stirred under hydrogen atmosphere (80 psi) for 12 h at room temperature. The reaction mixture was diluted with THF, filtered through a pad of celite, and washed with an excess of 20% THF in DCM. The filtrate was collected and concentrated to afford tert-butyl 4-((1R)-1-(4-(3-(2,6-dioxopiperidin-3-yl)-1-methyl-1H-indazol-6-yl)piperidin-1-yl)ethyl)piperidine-1-carboxylate as a brown semi-solid (0.52 g) which was used in the next step without further purification.

LC-MS (ESI): m/z=538.21 [M+H]⁺

### Preparation of 3-(1-methyl-6-(1-((R)-1-(piperidin-4-yl)ethyl)piperidin-4-yl)-1H-indazol-3-yl)piperidine-2,6-dione

To a stirred solution tert-butyl 4-((1R)-1-(4-(3-(2,6-dioxopiperidin-3-yl)-1-methyl-1H-indazol-6-yl)piperidin-1-yl)ethyl)piperidine-1-carboxylate (0.52 g, 1.18 mmol)) in DCM (10.4 mL) cooled to 0° C. was added TFA (2.6 mL). The mixture was stirred at room temperature for 2 h. The reaction mixture was concentrated under reduced pressure and co-distilled with diethyl ether to get a crude product which was washed with n-pentane to afford 3-(1-methyl-6-(1-((R)-1-(piperidin-4-yl)ethyl)piperidin-4-yl)-1H-indazol-3-yl)piperidine-2,6-dione (0.50 g) as a semi-solid.

LC-MS (ESI): m/z=438.52 [M+H]⁺

### Preparation of 3-(6-(1-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione

### [3-(6-(1-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione]

To a stirred solution of 3-(1-methyl-6-(1-((R)-1-(piperidin-4-yl)ethyl)piperidin-4-yl)-1H-indazol-3-yl)piperidine-2,6-dione (0.50 g, 1.14 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.21 g, 0.45 mmol) in DMSO (4.0 mL) was added N,N-diisopropylethylamine (0.73 g, 5.7 mmol). The mixture was stirred at 100° C. for 6 h. The reaction mixture was quenched with ice-cold water. The precipitated solid was filtered and dried under vacuum. The crude product was purified by prep-HPLC to afford 3-(6-(1-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione [3-(6-(1-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperidin-4-yl)-1-methyl-1H-indazol-3-yl)piperidine-2,6-dione](16.8 mg) as an off-white solid.

LC-MS (ESI): m/z=869.43 [M+H]⁺

¹H NMR (400 MHz, DMSO-d6): δ 10.89 (s, 1H), 8.76 (s, 1H), 8.23-8.22 (m, 2H), 8.03 (s, 1H), 7.74 (d, J=9.20 Hz, 1H), 7.60 (d, J=8.40 Hz, 1H), 7.45 (d, J=9.20 Hz, 1H), 7.40 (s, 1H), 7.02 (d, J=8.40 Hz, 1H), 6.18 (s, 1H), 4.50-4.30 (m, 5H), 3.97 (s, 3H), 3.57 (s, 3H), 3.33-3.35 (br s, 1H), 2.76-2.56 (m, 8H), 2.36-2.35 (m, 1H), 2.26-2.24 (m, 1H), 2.18-2.15 (m, 2H), 2.13-2.07 (m, 1H), 1.76-1.67 (m, 6H), 1.02-0.90 (m, 5H), 0.73-0.71 (m, 1H), 0.53-0.50 (m, 2H), 0.36-0.34 (m, 1H).

### Example 162: 3-(6-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione

### [3-(6-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione](Compound 523a)

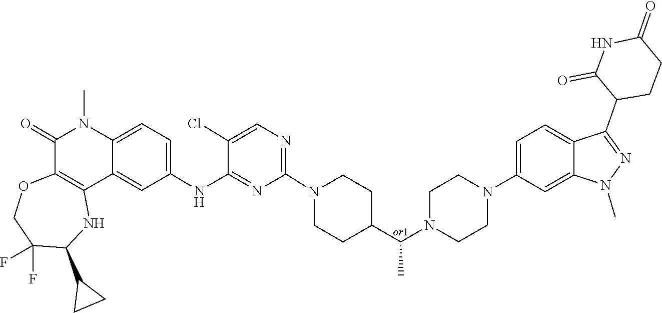 [SMILES: C[C@H](C1CCN(c2ncc(Cl)c(Nc3ccc4c(c3)c3c(c(=O)n4C)OCC(F)(F)[C@H](C4CC4)N3)n2)CC1)N1CCN(c2ccc3c(C4CCC(=O)NC4=O)nn(C)c3c2)CC1]

### Preparation of tert-butyl (R)-4-(1-(piperazin-1-yl)ethyl)piperidine-1-carboxylate

To a stirred solution of benzyl (R)-4-(1-(1-(tert-butoxycarbonyl)piperidin-4-yl)ethyl)piperazine-1-carboxylate (0.95 g, 2.2 mmol) in THF (47.5 mL) was added 20% Pd(OH)₂ on carbon (0.95 g, 100% w/w). The mixture was stirred under hydrogen atmosphere (80 psi) at room temperature for 12 h. The reaction mixture was filtered through a celite bed and washed with THF. The combined organic solvents were concentrated under reduced pressure to afford tert-butyl (R)-4-(1-(piperazin-1-yl)ethyl)piperidine-1-carboxylate (0.8 g) as a brown solid which was used in the next step without further purification.

LC-MS (ESI): m/z=298.20 [M+H]⁺

### Preparation of tert-butyl (R)-4-(1-(4-(3-(2,6-bis(benzyloxy)pyridin-3-yl)-1-methyl-1H-indazol-6-yl)piperazin-1-yl)ethyl)piperidine-1-carboxylate

A solution tert-butyl (R)-4-(1-(piperazin-1-yl)ethyl)piperidine-1-carboxylate (0.6 g, 2.01 mmol), 3-(2,6-bis(benzyloxy)pyridin-3-yl)-6-bromo-1-methyl-1H-indazole (1.0 g, 2.01 mmol), and sodium tert-butoxide (0.58 g, 6.05 mmol) in 1,4-dioxane (12 mL) was degassed with argon for 5 minutes. To this mixture was added RuPhos (0.09 g, 0.20 mmol) and RuPhos-Pd-G3 (0.08 g, 0.10 mmol). The mixture was heated at 100° C. and stirred for 16 h. The reaction mixture was quenched with ice-cold water (50 mL) and extracted with ethyl acetate (3×100 mL). The combined organic layers were washed with brine solution (50 mL), dried over anhydrous Na₂SO₄, filtered, and concentrated under vacuum to get a crude product which was purified by column chromatography (40% ethyl acetate in petroleum ether) to afford tert-butyl (R)-4-(1-(4-(3-(2,6-bis(benzyloxy)pyridin-3-yl)-1-methyl-1H-indazol-6-yl)piperazin-1-yl)ethyl)piperidine-1-carboxylate (0.3 g) as a pale brown semi-solid.

LC-MS (ESI): m/z=717.64 [M+H]⁺

### Preparation of tert-butyl 4-((1R)-1-(4-(3-(2,6-dioxopiperidin-3-yl)-1-methyl-1H-indazol-6-yl)piperazin-1-yl)ethyl)piperidine-1-carboxylate

To a stirred solution of tert-butyl (R)-4-(1-(4-(3-(2,6-bis(benzyloxy)pyridin-3-yl)-1-methyl-1H-indazol-6-yl)piperazin-1-yl)ethyl)piperidine-1-carboxylate (0.25 g, 0.34 mmol) in THF (21 mL) was added 20% Pd (OH)₂ on carbon (0.25 g, 100% w/w). The mixture was stirred under hydrogen atmosphere (70 psi) at room temperature for 12 h. The reaction mixture was filtered through a celite bed, washed with THF (50 mL), and concentrated. The crude product was washed with n-pentane (100 mL) and dried to afford tert-butyl 4-((1R)-1-(4-(3-(2,6-dioxopiperidin-3-yl)-1-methyl-1H-indazol-6-yl)piperazin-1-yl)ethyl)piperidine-1-carboxylate (0.25 g) as a brown solid which was used in the next step without further purification.

LC-MS (ESI): m/z=539.37 [M+H]⁺

### Preparation of 3-(1-methyl-6-(4-((R)-1-(piperidin-4-yl)ethyl)piperazin-1-yl)-1H-indazol-3-yl)piperidine-2,6-dione

To a stirred solution of tert-butyl 4-((1R)-1-(4-(3-(2,6-dioxopiperidin-3-yl)-1-methyl-1H-indazol-6-yl)piperazin-1-yl)ethyl)piperidine-1-carboxylate (0.25 g, 0.46 mmol) in DCM (2.5 mL) at 0° C. was added TFA (1.25 mL). The mixture was stirred at room temperature for 2 h. The reaction mixture was concentrated under reduced pressure to obtain crude product which was triturated with n-pentane (10 ml) to afford 3-(1-methyl-6-(4-((R)-1-(piperidin-4-yl)ethyl)piperazin-1-yl)-1H-indazol-3-yl)piperidine-2,6-dione (0.24 g) as a grey solid.

LC-MS (ESI): m/z=439.53 [M+H]⁺

### Preparation of 3-(6-(4-((R)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione

### [3-(6-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione]

To a stirred solution of 3-(1-methyl-6-(4-((R)-1-(piperidin-4-yl)ethyl)piperazin-1-yl)-1H-indazol-3-yl)piperidine-2,6-dione (0.24 g, 0.54 mmol) in DMSO (2.4 mL) were added N,N-diisopropylethylamine (2.4 mL) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.128 g, 0.27 mmol). The mixture was stirred at 100° C. for 6 h. The reaction mixture was concentrated under reduced pressure and purified by prep-HPLC to afford 3-(6-(4-((R)-1-(1-(5-chloro-4-(((S)-2-cyclopropyl-3,3-difluoro-7-methyl-6-oxo-1,2,3,4,6,7-hexahydro-[1,4]oxazepino [2,3-c]quinolin-10-yl)amino)pyrimidin-2-yl)piperidin-4-yl)ethyl)piperazin-1-yl)-1-methyl-1H-indazol-3-yl)piperidine-2,6-dione [3-(6-(4-((R*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione](0.108 g) as an off-white solid.

LC-MS (ESI): m/z=870.38 [M+H]⁺¹HNMR (400 MHz, DMSO-d₆): δ 10.85 (s, 1H), 8.75 (s, 1H), 8.19 (d, J=2.00 Hz, 1H), 8.16 (s, 1H), 8.02 (s, 1H), 7.74 (dd, J=7.2, 1.6 Hz, 1H), 7.46 (dd, 10.40, 8.80 Hz, 2H), 6.90 (d, J=8.80 Hz, 1H), 6.82 (s, 1H), 6.17 (br s, 1H), 4.60-4.31 (m, 4H), 4.30-4.20 (m, 1H), 3.88 (s, 3H), 3.55 (s, 3H), 3.21 (br s, 6H), 2.80-2.65 (m, 3H), 2.60-2.50 (m, 3H), 2.41-2.30 (m, 2H), 2.21-2.15 (m, 1H), 2.15-2.0 (m, 1H), 1.70-1.61 (m, 1H), 1.60-1.51 (m, 1H), 1.40-1.30 (m, 1H), 1.10-1.00 (m, 2H), 0.91 (d, J=6.40 Hz, 3H), 0.81-0.75 (m, 1H), 0.63-0.50 (m, 2H), 0.41-0.32 (m, 1H).

### Example 163: 3-(6-(4-((S)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione

### [3-(6-(4-((S*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione](Compound 523b)

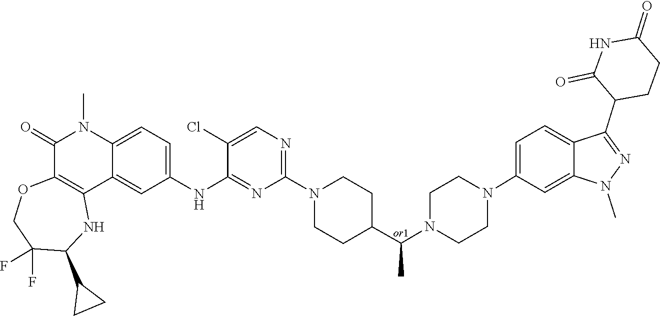 [SMILES: C[C@@H](C1CCN(c2ncc(Cl)c(Nc3ccc4c(c3)c3c(c(=O)n4C)OCC(F)(F)[C@H](C4CC4)N3)n2)CC1)N1CCN(c2ccc3c(C4CCC(=O)NC4=O)nn(C)c3c2)CC1]

### Preparation of tert-butyl (S)-4-(1-(piperazin-1-yl)ethyl)piperidine-1-carboxylate

To a stirred solution of benzyl (S)-4-(1-(1-(tert-butoxycarbonyl)piperidin-4-yl)ethyl)piperazine-1-carboxylate (0.95 g, 2.2 mmol) in THF (47.5 mL) was added 20% Pd(OH)₂ on carbon (0.95 g, 100% w/w). The mixture was stirred under hydrogen atmosphere (80 psi) at room temperature for 12 h. The reaction mixture was filtered through a celite bed and washed with THF. The combined organic solvents were concentrated under reduced pressure to get tert-butyl (S)-4-(1-(piperazin-1-yl)ethyl)piperidine-1-carboxylate (0.8 g) as a brown solid which was used in the next step without further purification.

LC-MS (ESI): m/z=298.27 [M+H]⁺

### Preparation of tert-butyl (S)-4-(1-(4-(3-(2,6-bis(benzyloxy)pyridin-3-yl)-1-methyl-1H-indazol-6-yl)piperazin-1-yl)ethyl)piperidine-1-carboxylate

A stirred solution of tert-butyl (S)-4-(1-(piperazin-1-yl)ethyl)piperidine-1-carboxylate (0.6 g, 2.01 mmol), 3-(2,6-bis(benzyloxy)pyridin-3-yl)-6-bromo-1-methyl-1H-indazole (1.0 g, 2.01 mmol), and sodium tert-butoxide (0.58 g, 6.05 mmol) in 1,4-dioxane (12 mL) was degassed with argon for 5 minutes. To this mixture was added RuPhos-Pd-G3 (0.08 g, 0.10 mmol). The mixture was heated at 100° C. and stirred for 16 h. The reaction mixture was quenched with ice-cold water (50 mL) and extracted with ethyl acetate (3×100 mL). The combined organic layers were washed with brine (50 mL), dried over anhydrous Na₂SO₄, filtered, and concentrated under vacuum to get a crude product which was purified by column chromatography (40% ethyl acetate in petroleum ether) to afford tert-butyl (S)-4-(1-(4-(3-(2,6-bis(benzyloxy)pyridin-3-yl)-1-methyl-1H-indazol-6-yl)piperazin-1-yl)ethyl)piperidine-1-carboxylate (0.3 g) as a pale brown semi-solid.

LC-MS (ESI): m/z=717.37 [M+H]⁺

### Preparation of tert-butyl 4-((1S)-1-(4-(3-(2,6-dioxopiperidin-3-yl)-1-methyl-1H-indazol-6-yl)piperazin-1-yl)ethyl)piperidine-1-carboxylate

To a stirred solution of tert-butyl (S)-4-(1-(4-(3-(2,6-bis(benzyloxy)pyridin-3-yl)-1-methyl-1H-indazol-6-yl)piperazin-1-yl)ethyl)piperidine-1-carboxylate (0.25 g, 0.34 mmol) in THF (21 mL) was added 20% Pd(OH)₂ on carbon (0.25 g, 100% w/w). The mixture was stirred under hydrogen atmosphere (80 psi) at room temperature for 12 h. The reaction mixture was filtered through a celite bed, washed with THF (50 mL), and concentrated completely. The crude product was washed with n-pentane (100 mL) and dried to afford tert-butyl 4-((1S)-1-(4-(3-(2,6-dioxopiperidin-3-yl)-1-methyl-1H-indazol-6-yl)piperazin-1-yl)ethyl)piperidine-1-carboxylate (0.25 g) as a brown solid which was used in the next step without further purification.

LC-MS (ESI): m/z=539.51 [M+H]⁺

### Preparation of 3-(1-methyl-6-(4-((S)-1-(piperidin-4-yl)ethyl)piperazin-1-yl)-1H-indazol-3-yl)piperidine-2,6-dione

To a stirred solution of tert-butyl 4-((1S)-1-(4-(3-(2,6-dioxopiperidin-3-yl)-1-methyl-1H-indazol-6-yl)piperazin-1-yl)ethyl)piperidine-1-carboxylate (0.25 g, 0.46 mmol) in DCM (2.5 mL) at 0° C. was added TFA (1.25 mL). The mixture was stirred at room temperature for 2 h. The reaction mixture was concentrated under reduced pressure to obtain a crude product, which was triturated with n-pentane (10 ml) to afford 3-(1-methyl-6-(4-((S)-1-(piperidin-4-yl)ethyl)piperazin-1-yl)-1H-indazol-3-yl)piperidine-2,6-dione (0.15 g) as a grey solid.

LC-MS (ESI): m/z=439.57 [M+H]⁺

### Preparation of 3-(6-(4-((S)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione

### [3-(6-(4-((S*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione]

To a stirred solution of 3-(1-methyl-6-(4-((S)-1-(piperidin-4-yl)ethyl)piperazin-1-yl)-1H-indazol-3-yl)piperidine-2,6-dione (0.15 g, 0.34 mmol) in DMSO (1.5 mL) were added N,N-diisopropylethylamine (1.5 mL) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.06 g, 0.13 mmol). The mixture and stirred at 100° C. for 6 h. The reaction mixture was concentrated under reduced pressure and purified by prep-HPLC to afford 3-(6-(4-((S)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione [3-(6-(4-((S*)-1-(1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)ethyl)piperazin-1-yl)-1-methyl-1H-indazol-3-yl)piperidine-2,6-dione](0.011 g) as an off-white solid.

LC-MS (ESI): m/z=870.46 [M+H]⁺¹HNMR (400 MHz, DMSO-d₆): δ 10.84 (s, 1H), 8.75 (s, 1H), 8.29 (br s, 1H), 8.19 (d, J=1.6 Hz, 1H), 8.02 (s, 1H), 7.73 (dd, J=7.2, 2.0 Hz, 1H), 7.46 (dd, J=10.4, 8.8 Hz, 2H), 6.90 (dd, J=7.2, 8.80 Hz, 1H), 6.81 (s, 1H), 6.18 (br s, 1H), 4.48-4.29 (m, 3H), 4.28-4.21 (m, 1H), 3.88 (s, 3H), 3.55 (s, 3H), 3.21 (br s, 3H), 2.80-2.65 (m, 3H), 2.63-2.55 (m, 2H), 2.45-2.42 (m, 2H), 2.29-2.25 (m, 2H), 2.19-2.15 (m, 1H), 2.15-2.0 (m, 1H), 1.72-1.63 (m, 1H), 1.62-1.51 (m, 1H), 1.39-1.30 (m, 2H), 1.25 (s, 1H), 1.09-0.89 (m, 6H), 0.75-0.70 (m, 1H), 0.55-0.50 (m, 2H), 0.41-0.32 (m, 1H).

### Example 164: 3-(6-(1-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione (Compound 525a)

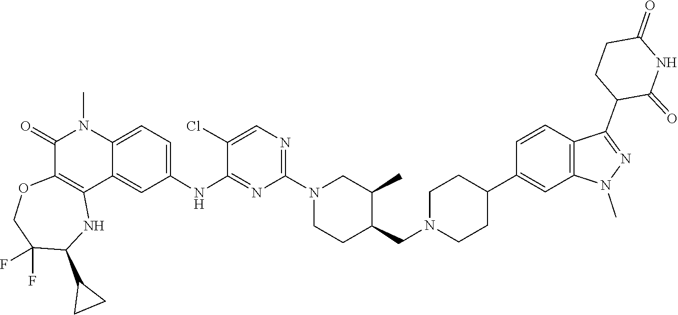 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCC(c2ccc3c(C4CCC(=O)NC4=O)nn(C)c3c2)CC1]

### Preparation of tert-butyl (3S, 4S)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate

To a stirred solution of (3S, 4S)-1-(tert-butoxycarbonyl)-3-methylpiperidine-4-carboxylic acid (0.45 g, 1.85 mmol) and 3-(2,6-bis(benzyloxy)pyridin-3-yl)-1-methyl-6-(1,2,3,6-tetrahydropyridin-4-yl)-1H-indazole hydrochloride (0.60 g, 1.11 mmol in DMF (9.0 mL) was added N,N-diisopropylethylamine (0.95 mL, 5.56 mmol). The mixture was stirred at room temperature for 5 min followed by the addition of HATU (1.05 g, 2.78 mmol) at 0° C. The mixture was stirred at room temperature for 2 h. The reaction mixture was poured into ice-cold water and stirred for 10 min. The precipitated solid was filtered and dried to afford tert-butyl (3S, 4S)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate (0.60 g) as an off white solid.

LC-MS (ESI): m/z=728.75 [M+H]⁺

### Preparation of tert-butyl (3S, 4S)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate

To a stirred solution of tert-butyl (3S, 4S)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate (0.60 g, 0.82 mmol)) in DCM (12.0 mL) was added 2 M LiAlH₄ in THF (0.90 mL) at 0° C. The mixture was allowed to stir at room temperature for 1 h. The reaction mixture was quenched with saturated solution of ammonium chloride (30 mL) and extracted with ethyl acetate (50 mL). The organic layer was dried over sodium sulfate and concentrated under reduced pressure to afford tert-butyl (3S, 4S)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate (0.60 g) as an off-white solid.

LC-MS (ESI): m/z=714.41 [M+H]⁺

### Preparation of tert-butyl (3S, 4S)-4-((4-(3-(2,6-dioxopiperidin-3-yl)-1-methyl-1H-indazol-6-yl)piperidin-1-yl)methyl)-3-methylpiperidine-1-carboxylate

To a stirred solution of tert-butyl (3S, 4S)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate (0.55 g, 0.77 mmol) in THF (33 mL) was added 20% Pd(OH)₂ (0.55 g) at room temperature. The reaction mixture was stirred under hydrogen pressure (60 psi) at room temperature for 16 h. The reaction mixture was diluted with THF (100 mL), filtered through a celite bed, and washed with THF (200 mL). The filtrate was concentrated and dried to afford tert-butyl (3S, 4S)-4-((4-(3-(2,6-dioxopiperidin-3-yl)-1-methyl-1H-indazol-6-yl)piperidin-1-yl)methyl)-3-methylpiperidine-1-carboxylate (0.41 g) as an off-white solid.

LC-MS (ESI): m/z=538.43 [M+H]⁺

### Preparation of 3-(1-methyl-6-(1-(((3S, 4S)-3-methylpiperidin-4-yl)methyl)piperidin-4-yl)-1H-indazol-3-yl)piperidine-2,6-dione

To a stirred solution of tert-butyl (3S, 4S)-4-((4-(3-(2,6-dioxopiperidin-3-yl)-1-methyl-1H-indazol-6-yl)piperidin-1-yl)methyl)-3-methylpiperidine-1-carboxylate (0.4 g, 0.74 mmol) in DCM (8.0 mL) was added TFA (2.0 mL) at 0° C. The mixture was stirred at room temperature for 2 h. The reaction mixture was concentrated, and the crude product was triturated with diethyl ether (20 mL) to afford 3-(1-methyl-6-(1-(((3S, 4S)-3-methylpiperidin-4-yl)methyl)piperidin-4-yl)-1H-indazol-3-yl)piperidine-2,6-dione (0.40 g, crude) as an off white solid.

LC-MS (ESI): m/z=438.27 [M+H]⁺

### Preparation of 3-(6-(1-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione

To a stirred solution of 3-(1-methyl-6-(1-(((3S, 4S)-3-methylpiperidin-4-yl)methyl)piperidin-4-yl)-1H-indazol-3-yl)piperidine-2,6-dione (0.40 g, 0.91 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.13 g, 0.27 mmol) in DMSO (4.0 mL) was added N,N-diisopropylethylamine (0.78 mL, 4.58 mmol). The resulting reaction mixture was stirred at 100° C. for 10 h. The reaction mixture was poured into ice cold water (50 mL), and the precipitate thus formed was filtered and dried under reduced pressure. The crude product was purified by prep-HPLC to afford 3-(6-(1-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione (0.080 g) as a yellow solid.

LC-MS (ESI): m/z=869.39 [M+H]⁺

¹H NMR (400 MHz, DMSO-d6): δ 10.99 (s, 1H), 8.72 (s, 1H), 8.15 (s, 1H), 8.00 (s, 1H), 7.77 (dd, J=1.60, 9.00 Hz, 1H), 7.60 (d, J=8.40 Hz, 1H), 7.45-7.43 (m, 2H), 7.03 (d, J=8.40 Hz, 1H), 6.17 (s, 1H), 4.42-4.30 (m, 5H), 3.97 (s, 3H), 3.57 (s, 3H), 3.32-3.02 (m, 1H), 2.95-2.92 (m, 2H), 2.67-2.65 (m, 1H), 2.60-2.57 (m, 2H), 2.50-2.49 (m, 1H), 2.36-2.33 (m, 3H), 2.01-1.98 (m, 9H), 1.75-1.73 (m, 4H), 1.50-1.29 (m, 3H), 0.76-0.74 (m, 1H), 0.52-0.50 (m, 2H), 0.39-0.36 (m, 1H).

### Example 165: 3-(6-(1-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione (Compound 525b)

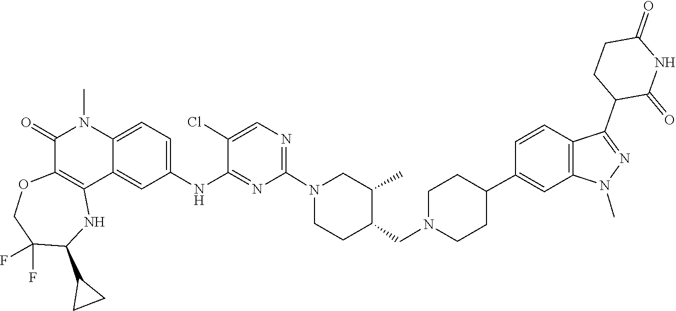 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCC(c2ccc3c(C4CCC(=O)NC4=O)nn(C)c3c2)CC1]

### Preparation of tert-butyl (3R, 4R)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate

To a stirred solution of 3-(2,6-bis(benzyloxy)pyridin-3-yl)-1-methyl-6-(1,2,3,6-tetrahydropyridin-4-yl)-1H-indazole (1.0 g, 1.99 mmol.) and (3R, 4R)-1-(tert-butoxycarbonyl)-3-methylpiperidine-4-carboxylic acid (0.38 g, 1.59 mmol.) in DMF (10.0 mL) was added DIPEA (1.73 mL, 9.94 mmol). The mixture was stirred for 5 min, followed by the addition of HATU (1.36 g, 3.58 mmol.) at 0° C. The mixture was stirred at room temperature for 2 h. The reaction mixture was poured into ice-cold water (5 mL), and the precipitated solid was filtered and dried to afford tert-butyl (3R, 4R)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate (1.1 g) as an off white solid.

LC-MS (ESI): m/z=728.73 [M+H]⁺

### Preparation of tert-butyl (3R, 4R)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate

To a stirred solution of tert-butyl (3R, 4R)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate (1.1 g, 1.51 mmol) in THF (30.0 mL) was added 2M LiAlH₄ in THF (3.0 mL) at 0° C. The mixture was stirred at 60° C. for 3 h. The reaction mixture was quenched with ammonium chloride solution (10 mL) and extracted with ethyl acetate (30 mL). The organic layer was dried to afford tert-butyl (3R, 4R)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate (1.2 g) as an off-white semi-solid.

LC-MS (ESI): m/z=714.38 [M+H]⁺

### Preparation of tert-butyl (3R, 4R)-4-((4-(3-(2,6-dioxopiperidin-3-yl)-1-methyl-1H-indazol-6-yl)piperidin-1-yl)methyl)-3-methylpiperidine-1-carboxylate

To a stirred solution of tert-butyl (3R, 4R)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate (1.2 g, 1.68 mmol.) in THF (240 mL) was added 20% palladium hydroxide (2.36 g, 16.80 mmol.). The mixture was stirred under H₂ (80 psi) pressure for 16 h at room temperature. The reaction mixture was diluted with THF (30 mL), filtered through a celite bed, and washed with THF:DCM (1:1, 20 mL). The filtrate was concentrated and dried to obtain crude product which was triturated with diethyl ether to afford tert-butyl (3R, 4R)-4-((4-(3-(2,6-dioxopiperidin-3-yl)-1-methyl-1H-indazol-6-yl)piperidin-1-yl)methyl)-3-methylpiperidine-1-carboxylate (1.0 g) as a light brown semi-solid.

LC-MS (ESI): m/z=538.66 [M+H]⁺

### Preparation of 3-(1-methyl-6-(1-(((3R, 4R)-3-methylpiperidin-4-yl)methyl)piperidin-4-yl)-1H-indazol-3-yl)piperidine-2,6-dione

To a stirred solution of tert-butyl (3R, 4R)-4-((4-(3-(2,6-dioxopiperidin-3-yl)-1-methyl-1H-indazol-6-yl)piperidin-1-yl)methyl)-3-methylpiperidine-1-carboxylate (1.0 g, 2.28 mmol) in DCM (20.0 mL) was added TFA (5.0 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL) to afford 3-(1-methyl-6-(1-(((3R, 4R)-3-methylpiperidin-4-yl)methyl)piperidin-4-yl)-1H-indazol-3-yl)piperidine-2,6-dione (0.8 g) as a light-brown solid.

LC-MS (ESI): m/z=438.46 [M+H]⁺

### Preparation of 3-(6-(1-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione

To a stirred solution of 3-(1-methyl-6-(1-(((3R, 4R)-3-methylpiperidin-4-yl)methyl)piperidin-4-yl)-1H-indazol-3-yl)piperidine-2,6-dione (0.8 g, 1.82 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.17 g, 0.36 mmol) in DMSO (8.0 mL) was added DIPEA (1.5 mL, 9.14 mmol). The mixture was stirred at 120° C. for 16 h. The reaction mixture was cooled to room temperature and poured into ice water. The precipitated solid was filtered and dried to obtain crude product which was purified by prep-HPLC to afford 3-(6-(1-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione (0.067 g) as a pale brown solid.

LC-MS (ESI): m/z=869.53 [M+H]⁺

¹H NMR (400 MHz, DMSO-d6): δ 10.88 (s, 1H), 8.75 (s, 1H), 8.17 (s, 2H), 8.00 (s, 1H), 7.76 (d, J=8.80 Hz, 1H), 7.60 (d, J=8.40 Hz, 1H), 7.47-7.43 (m, 2H), 7.03 (d, J=8.00 Hz, 1H), 6.18 (s, 1H), 4.46-4.30 (m, 5H), 3.97 (s, 3H), 3.57 (s, 3H), 3.00-2.85 (m, 5H), 2.67-2.60 (m, 4H), 2.36-2.33 (m, 1H), 2.18-2.17 (m, 3H), 2.01-1.92 (m, 4H), 1.78 (br s, 4H), 1.40-1.35 (m, 1H), 0.76-0.73 (m, 4H), 0.53-0.51 (m, 2H), 0.36-0.33 (m, 1H).

### Example 166: 3-(6-(1-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione (Compound 525c)

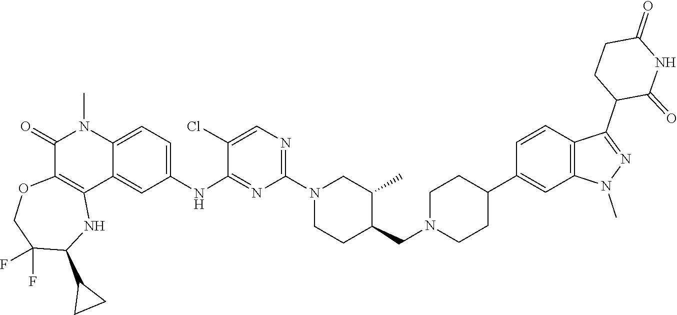 [SMILES: C[C@H]1CN(c2ncc(Cl)c(Nc3ccc4c(c3)c3c(c(=O)n4C)OCC(F)(F)[C@H](C4CC4)N3)n2)CC[C@@H]1CN1CCC(c2ccc3c(C4CCC(=O)NC4=O)nn(C)c3c2)CC1]

### Preparation of tert-butyl (3R, 4S)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate

To a stirred solution of 3-(2,6-bis(benzyloxy)pyridin-3-yl)-1-methyl-6-(1,2,3,6-tetrahydropyridin-4-yl)-1H-indazole hydrochloride (0.7 g, 1.29 mmol), and (3R, 4S)-1-(tert-butoxycarbonyl)-3-methylpiperidine-4-carboxylic acid (0.25 g, 1.03 mmol) in DMF (14.0 mL) was added N,N-diisopropylethylamine (1.13 mL, 6.49 mmol). The mixture was stirred at room temperature for 5 min followed by the addition of HATU (0.88 g, 2.33 mmol) at 0° C. The mixture was stirred at room temperature for 2 h. The reaction mixture was poured in ice-cold water and stirred for 10 min. The precipitated solid was filtered and dried to afford tert-butyl (3R, 4S)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate (0.5 g) as an off-white solid.

LC-MS (ESI): m/z=728.75 [M+H]⁺

### Preparation of tert-butyl (3R, 4S)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate

To a stirred solution of tert-butyl (3R, 4S)-4-(4-(3-(2,6-bis(benzyloxy)pyridin-3-yl)-1-methyl-1H-indazol-6-yl)-1,2,3,6-tetrahydropyridine-1-carbonyl)-3-methylpiperidine-1-carboxylate (0.5 g, 0.68 mmol)) in DCM (10.0 mL) cooled to 0° C. was added 2 M LiAlH₄ in THF (0.75 mL). The mixture was stirred at room temperature for 1 h. The reaction mixture was quenched with saturated ammonium chloride (10 mL) and extracted with ethyl acetate (30 mL). The organic layer was dried over sodium sulfate and concentrated under reduced pressure to afford tert-butyl (3R, 4S)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate as an off-white semi-solid.

LC-MS (ESI): m/z=714.78 [M+H]⁺

### Preparation of tert-butyl (3R, 4S)-4-((4-(3-(2,6-dioxopiperidin-3-yl)-1-methyl-1H-indazol-6-yl)piperidin-1-yl)methyl)-3-methylpiperidine-1-carboxylate

To a stirred solution of tert-butyl (3R, 4S)-4-((4-(3-(2,6-bis(benzyloxy)pyridin-3-yl)-1-methyl-1H-indazol-6-yl)-3,6-dihydropyridin-1(2H)-yl)methyl)-3-methylpiperidine-1-carboxylate (0.5 g, 0.70 mmol) in THF (30 mL) was added 20% Pd(OH)₂ (0.5 g). The mixture was stirred under hydrogen pressure (20 psi) at room temperature for 12 h. The reaction mixture was diluted with THF (100 mL) and filtered through a celite bed which was washed with THF (200 mL). The filtrate was concentrated and dried to afford tert-butyl (3R, 4S)-4-((4-(3-(2,6-dioxopiperidin-3-yl)-1-methyl-1H-indazol-6-yl)piperidin-1-yl)methyl)-3-methylpiperidine-1-carboxylate (0.4 g) as a light-brown semi-solid.

LC-MS (ESI): m/z=538.48 [M+H]⁺

### Preparation of 3-(1-methyl-6-(1-(((3R, 4S)-3-methylpiperidin-4-yl)methyl)piperidin-4-yl)-1H-indazol-3-yl)piperidine-2,6-dione

To a stirred solution of tert-butyl (3R, 4S)-4-((4-(3-(2,6-dioxopiperidin-3-yl)-1-methyl-1H-indazol-6-yl)piperidin-1-yl)methyl)-3-methylpiperidine-1-carboxylate (0.4 g, 0.74 mmol) in DCM (4.0 mL) was added TFA (2.0 mL) at 0° C. The mixture was stirred at room temperature for 1 h. The reaction mixture was concentrated to get crude product which was triturated with diethyl ether (15 mL) and dried to afford 3-(1-methyl-6-(1-(((3R, 4S)-3-methylpiperidin-4-yl)methyl)piperidin-4-yl)-1H-indazol-3-yl)piperidine-2,6-dione (0.35 g) as a light-brown solid.

LC-MS (ESI): m/z=438.39 [M+H]⁺

### Preparation of 3-(6-(1-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione

To a stirred solution of 3-(1-methyl-6-(1-(((3R, 4S)-3-methylpiperidin-4-yl)methyl)piperidin-4-yl)-1H-indazol-3-yl)piperidine-2,6-dione (0.35 g, 0.79 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.11 g, 0.23 mmol) in DMSO (7.0 mL) was added N,N-diisopropylethylamine (0.69 mL, 3.99 mmol). The mixture was stirred at 100° C. for 3 h. The reaction mixture was poured into ice cold water (10 mL) and the solid precipitate was filtered. The crude product was purified by prep-HPLC to afford 3-(6-(1-(((3R, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione (0.080 g) as an off-white solid.

LC-MS (ESI): m/z=869.49 [M+H]⁺

¹H NMR (400 MHz, DMSO-d6): δ 10.89 (s, 1H), 8.79 (s, 1H), 8.18 (d, J=3.20 Hz, 1H), 8.03 (s, 1H), 7.75 (d, J=8.40 Hz, 1H), 7.60 (d, J=8.40 Hz, 1H), 7.46-7.42 (m, 2H), 7.02 (d, J=8.40 Hz, 1H), 6.21 (s, 1H), 4.37-4.30 (m, 5H), 3.96 (s, 3H), 3.57 (s, 3H), 3.20-3.10 (br, 3H), 3.00-2.99 (m, 1H), 2.86-2.84 (m, 2H), 2.60-2.61 (m, 2H), 2.36-2.39 (m, 2H), 2.16-2.13 (m, 3H), 1.77-1.86 (m, 6H), 1.35-1.24 (m, 4H), 1.01-1.00 (m, 1H), 0.87-0.88 (d, J=7.20 Hz, 3H), 0.71-0.70 (m, 3H), 0.53-0.50 (m, 2H), 0.36-0.34 (m, 1H).

### Example 167: 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-5-fluoro-1-methyl-1H-indazol-3-yl)piperidine-2,6-dione (Compound 531a)

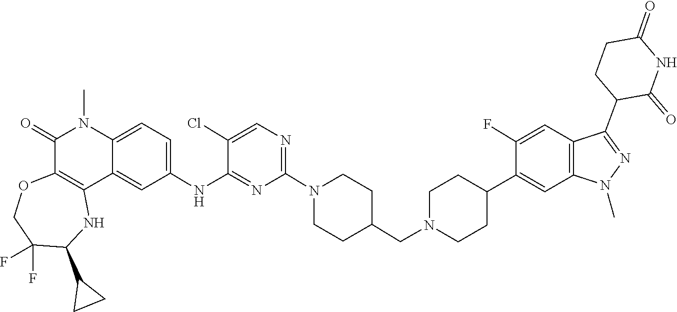 [SMILES: Cn1nc(C2CCC(=O)NC2=O)c2cc(F)c(C3CCN(CC4CCN(c5ncc(Cl)c(Nc6ccc7c(c6)c6c(c(=O)n7C)OCC(F)(F)[C@H](C7CC7)N6)n5)CC4)CC3)cc21]

### Preparation of 6-bromo-5-fluoro-1H-indazole

To a stirred solution of 4-bromo-2,5-difluorobenzaldehyde (5.00 g, 22.62 mmol) in DME (50 mL), methoxyamine hydrochloride (2.00 g, 23.98 mmol) and potassium carbonate (3.00 g, 21.72 mmol) were added. The mixture was heated at 40° C. for 3 h. The mixture was diluted with DME (100 mL) and filtered through a pad of celite and washed with excess of ethyl acetate (100 mL).

The collected filtrate was concentrated under vacuum to obtain a crude product. Hydrazine monohydrate, 98+% (12.5 mL) in DME (50 mL) was added to the above crude product and heated to 90° C. for 18 h. The reaction mixture was concentrated under reduced pressure to afford crude product which was purified by flash chromatography (10-15% of ethyl acetate in petroleum ether) to obtain 6-bromo-5-fluoro-1H-indazole (1.30 g) as a pale-yellow solid.

LC-MS (ESI): m/z=214.85 [M+H]⁺

### Preparation of 6-bromo-5-fluoro-3-iodo-1H-indazole

To stirred solution of 6-bromo-5-fluoro-1H-indazole (1.30 g, 6.05 mmol) in THF (25 mL), were added iodine (3.07 g, 12.09 mmol) and potassium hydroxide (1.70 g, 30.23 mmol) at room temperature. The mixture was stirred at room temperature for 2 h. The reaction mixture was concentrated under reduced pressure to afford a crude product which was triturated with n-pentane to afford 6-bromo-5-fluoro-3-iodo-1H-indazole (0.90 g) as a pale-brown solid.

LC-MS (ESI): m/z=340.84 [M+H]⁺

### Preparation of 6-bromo-5-fluoro-3-iodo-1-methyl-1H-indazole

To a stirred solution of 6-bromo-5-fluoro-3-iodo-1H-indazole (0.90 g, 2.64 mmol) in THF (10 mL) were added potassium tert-butoxide (0.89 g, 7.92 mmol) and iodomethane (1 mL) at 0° C.

The mixture was stirred at room temperature for 4 h. The reaction mixture was quenched with water (50 mL) and extracted with ethyl acetate (100 mL). The combined organic layers were washed with brine, dried over anhydrous Na₂SO₄, filtered, and concentrated under vacuum to obtain a crude product which was purified by flash chromatography (10-15% of ethyl acetate in petroleum ether) to obtain 6-bromo-5-fluoro-3-iodo-1-methyl-1H-indazole as an off-white solid (0.45 g).

LC-MS (ESI): m/z=354.84 [M+H]⁺

### Preparation of 3-(2,6-bis(benzyloxy)pyridin-3-yl)-6-bromo-5-fluoro-1-methyl-1H-indazole

To a stirred solution of 6-bromo-5-fluoro-3-iodo-1-methyl-1H-indazole (0.45 g, 1.27 mmol) and (2,6-bis(benzyloxy)pyridin-3-yl)boronic acid (0.51 g, 1.52 mmol) in dioxane (9 mL) and water (0.90 mL) was added K₃PO₄ (0.81 g, 3.80 mmol). The mixture was purged with N₂ for 15 min followed by the addition of Pd(PPh₃)₄(0.15 g, 0.13 mmol). The mixture was heated at 100° C. and stirred for 6 h. The reaction mixture was cooled to room temperature, diluted with ethyl acetate (100 mL), filtered through a celite bed, and washed with DCM (100 mL). The filtrate was concentrated to get a crude product which was purified by flash chromatography (5-10% of ethyl acetate in petroleum ether) to obtain 3-(2,6-bis(benzyloxy)pyridin-3-yl)-6-bromo-5-fluoro-1-methyl-1H-indazole as a pale-yellow solid (0.45 g).

LC-MS (ESI): m/z=520.35 [M+H]⁺

### Preparation of tert-butyl 4-(3-(2,6-bis(benzyloxy)pyridin-3-yl)-5-fluoro-1-methyl-1H-indazol-6-yl)-3,6-dihydropyridine-1(2H)-carboxylate

To a stirred solution of 3-(2,6-bis(benzyloxy)pyridin-3-yl)-6-bromo-5-fluoro-1-methyl-1H-indazole (0.45 g, 0.87 mmol) and tert-butyl 4-(4,4,5,5-tetramethyl-1,3,2-dioxaborolan-2-yl)-3,6-dihydropyridine-1(2H)-carboxylate (0.27 g, 0.87 mmol) in dioxane (4 mL) and water (0.90 mL) was added cesium carbonate (0.85 g, 2.60 mmol). The mixture was purged with N₂ for 15 min followed by the addition of Pd₂(dba)₃ (0.04 g, 0.04 mmol) and xantphos (0.05 g, 0.09 mmol).

The mixture was heated at 100° C. and stirred for 6 h. The reaction mixture was cooled to room temperature, diluted with ethyl acetate (200 mL), filtered through a celite bed, and washed with DCM (100 mL). The filtrate was concentrated to get a crude product which was purified by flash chromatography (5-10% of ethyl acetate in petroleum ether) to obtain tert-butyl 4-(3-(2,6-bis(benzyloxy)pyridin-3-yl)-5-fluoro-1-methyl-1H-indazol-6-yl)-3,6-dihydropyridine-1(2H)-carboxylate as a pale yellow semi-solid (0.40 g).

LC-MS (ESI): m/z=621.58 [M+H]⁺

### Preparation of 6-(benzyloxy)-5-(5-fluoro-1-methyl-6-(1,2,3,6-tetrahydropyridin-4-yl)-1H-indazol-3-yl)pyridin-2-ol

To a stirred solution of tert-butyl 4-(3-(2,6-bis(benzyloxy)pyridin-3-yl)-5-fluoro-1-methyl-1H-indazol-6-yl)-3,6-dihydropyridine-1(2H)-carboxylate (0.40 g, 0.64 mmol) in DCM (4 mL) at 0° C. was added TFA (3.20 mL). The mixture was stirred at room temperature for 2 h. The reaction mixture was concentrated under reduced pressure to afford crude 6-(benzyloxy)-5-(5-fluoro-1-methyl-6-(1,2,3,6-tetrahydropyridin-4-yl)-1H-indazol-3-yl)pyridin-2-ol as a pale brown semi-solid (0.45 g) which was used in the next step without further purification.

LC-MS (ESI): m/z=431.12 [M+H]⁺

### Preparation of tert-butyl 4-((4-(3-(2-(benzyloxy)-6-hydroxypyridin-3-yl)-5-fluoro-1-methyl-1H-indazol-6-yl)-3,6-dihydropyridin-1(2H)-yl)methyl)piperidine-1-carboxylate

To a stirred solution of 6-(benzyloxy)-5-(5-fluoro-1-methyl-6-(1,2,3,6-tetrahydropyridin-4-yl)-1H-indazol-3-yl)pyridin-2-ol (0.45 g, 1.04 mmol) and tert-butyl 4-formylpiperidine-1-carboxylate (0.24 g, 1.15 mmol) in THF (16 mL) were added sodium acetate (0.26 g, 3.14 mmol) and acetic acid (0.45 mL). The mixture was stirred at room temperature for 1 h followed by the addition of sodium triacetoxyborohydride (0.66 g, 3.14 mmol) at 0° C. The mixture was stirred at room temperature for 16 h. The reaction mixture was quenched with water (50 mL) and extracted with ethyl acetate (100 mL). The combined organic layers were washed with brine, dried over anhydrous Na₂SO₄, filtered, and concentrated under vacuum to obtain a crude product. The crude product was triturated with n-pentane to afford crude tert-butyl 4-((4-(3-(2-(benzyloxy)-6-hydroxypyridin-3-yl)-5-fluoro-1-methyl-1H-indazol-6-yl)-3,6-dihydropyridin-1(2H)-yl)methyl)piperidine-1-carboxylate (0.50 g) as a pale-yellow semi-solid.

LC-MS (ESI): m/z=718.33 [M+H]⁺

### Preparation of tert-butyl 4-((4-(3-(2,6-dioxopiperidin-3-yl)-5-fluoro-1-methyl-1H-indazol-6-yl)piperidin-1-yl)methyl)piperidine-1-carboxylate

To a stirred solution of tert-butyl 4-((4-(3-(2-(benzyloxy)-6-hydroxypyridin-3-yl)-5-fluoro-1-methyl-1H-indazol-6-yl)-3,6-dihydropyridin-1(2H)-yl)methyl)piperidine-1-carboxylate (0.40 g, 0.64 mmol) in THF (80 mL) was added 20% Pd(OH)₂ (0.40 g). The mixture was stirred under a hydrogen atmosphere (60 psi) at room temperature for 3 h. The reaction mixture was diluted with DCM (200 mL), washed with 30% THF:DCM (300 mL), and filtered through a celite bed. The collected filtrate was concentrated under vacuum to obtain crude product which was triturated with n-pentane to afford tert-butyl 4-((4-(3-(2,6-dioxopiperidin-3-yl)-5-fluoro-1-methyl-1H-indazol-6-yl)piperidin-1-yl)methyl)piperidine-1-carboxylate (0.23 g) as an off-white solid.

LC-MS (ESI): m/z=542.65 [M+H]⁺

### Preparation of 3-(5-fluoro-1-methyl-6-(1-(piperidin-4-ylmethyl)piperidin-4-yl)-1H-indazol-3-yl)piperidine-2,6-dione

To a stirred solution of tert-butyl 4-((4-(3-(2,6-dioxopiperidin-3-yl)-5-fluoro-1-methyl-1H-indazol-6-yl)piperidin-1-yl)methyl)piperidine-1-carboxylate (0.23 g, 0.42 mmol) in DCM (5 mL) at 0° C. was added TFA (1.84 mL). The mixture was stirred at room temperature for 2 h. The reaction mixture was concentrated under reduced pressure to afford 3-(5-fluoro-1-methyl-6-(1-(piperidin-4-ylmethyl)piperidin-4-yl)-1H-indazol-3-yl)piperidine-2,6-dione (0.25 g) as a pale-brown semi-solid which was used in the next step without further purification.

LC-MS (ESI): m/z=442.47 [M+H]⁺

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-5-fluoro-1-methyl-1H-indazol-3-yl)piperidine-2,6-dione

To a stirred solution of 3-(5-fluoro-1-methyl-6-(1-(piperidin-4-ylmethyl)piperidin-4-yl)-1H-indazol-3-yl)piperidine-2,6-dione (0.25 g, 0.56 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.17 mmol) in DMSO (5 mL) was added DIPEA (2.50 mL). The mixture was heated and stirred at 100° C. for 18 h. The reaction mixture was quenched with ice cold water (100 mL) and the precipitated solid was filtered and dried under vacuum to afford crude product which was purified by prep-HPLC to afford 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperidin-4-yl)-5-fluoro-1-methyl-1H-indazol-3-yl)piperidine-2,6-dione (0.038 g) as an off-white solid. ¹H NMR: (400 MHz, DMSO-d6): δ 10.89 (s, 1H), 8.17-8.16 (m, 1H), 8.07 (s, 1H), 7.75-7.72 (m, 1H), 7.56-7.54 (m, 1H), 7.44-7.41 (m, 2H), 7.20-6.86 (m, 1H), 6.24 (s, 1H), 4.45-4.31 (m, 4H), 4.01 (s, 2H), 3.62-3.60 (br, 4H), 3.26-3.15 (m, 4H), 3.07-3.01 (m, 2H), 2.85-2.83 (m, 2H), 2.64-2.60 (m, 2H), 2.49-2.40 (m, 1H), 2.18-2.08 (m, 6H), 1.75-1.72 (m, 2H), 1.35-1.31 (m, 3H), 1.24-1.20 (m, 1H), 1.15-1.10 (m, 2H), 0.74-0.70 (m, 1H), 0.52-0.51 (m, 2H), 0.32-0.30 (m, 1H).

LC-MS (ESI): m/z=873.41 [M+H]⁺

### Example 168: 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-fluoropiperidin-4-yl)-1-methyl-1H-indazol-3-yl)piperidine-2,6-dione (Compound 533a)

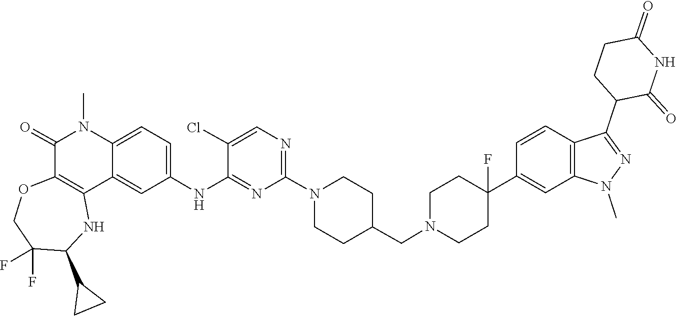 [SMILES: Cn1nc(C2CCC(=O)NC2=O)c2ccc(C3(F)CCN(CC4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-(3-(2,6-bis(benzyloxy)pyridin-3-yl)-1-methyl-1H-indazol-6-yl)-3,6-dihydropyridine-1(2H)-carboxylate

To a stirred solution of 3-(2,6-bis(benzyloxy)pyridin-3-yl)-6-bromo-1-methyl-1H-indazole (5.0 g, 9.992 mmol) and tert-butyl 4-(4,4,5,5-tetramethyl-1,3-dioxolan-2-yl)-3,6-dihydropyridine-1(2H)-carboxylate (3.70 g, 11.991 mmol) in 1,4-dioxane (200 mL) and water (100 mL) was added cesium carbonate (9.76 g, 29.976 mmol). The mixture was degassed for 10 min followed by the addition of Pd₂(dba)₃ (0.45 g, 0.500 mmol) and xantphos (0.57 g, 0.999 mmol). The mixture was stirred at 100° C. for 6 h. The reaction mixture was concentrated under reduced pressure. The resulting crude product was diluted with water (100 mL) and extracted with DCM (3×150 mL). The combined organic layers were washed with brine (100 mL), dried over anhydrous Na₂SO₄, filtered, and dried under vacuum to provide a crude product which was purified by column chromatography (30% ethyl acetate in petroleum ether) to afford tert-butyl 4-(3-(2,6-bis(benzyloxy)pyridin-3-yl)-1-methyl-1H-indazol-6-yl)-3,6-dihydropyridine-1(2H)-carboxylate (5.0 g) as a yellow solid.

LC-MS (ESI): m/z=603.43 [M+H]⁺

### Preparation of tert-butyl 4-(3-(2,6-bis(benzyloxy)pyridin-3-yl)-1-methyl-1H-indazol-6-yl)-4-hydroxypiperidine-1-carboxylate

To a stirred solution of tert-butyl 4-(3-(2,6-bis(benzyloxy)pyridin-3-yl)-1-methyl-1H-indazol-6-yl)-3,6-dihydropyridine-1(2H)-carboxylate (3.0 g, 4.97 mmol) in isopropyl alcohol (24.0 mL) and dichloromethane (6.0 mL) were added tris(2,2,6,6-tetramethyl-3,5-heptanedionato) manganese(III) (0.150 g, 0.249 mmol) and phenylsilane (1.857 mL, 14.93 mmol) at 0° C. under oxygen atmosphere. The mixture was stirred at 0° C. for 4 h. The reaction mixture was quenched with water (50 mL) and extracted with DCM (3×50 mL). The combined organic layers were washed with brine (50 mL), dried over anhydrous Na₂SO₄, filtered, and dried under vacuum to get a crude product which was purified by flash column chromatography (45% ethyl acetate in petroleum ether) to afford tert-butyl 4-(3-(2,6-bis(benzyloxy)pyridin-3-yl)-1-methyl-1H-indazol-6-yl)-4-hydroxypiperidine-1-carboxylate (2.0 g).

LC-MS (ESI): m/z=621.52 [M+H]⁺

### Preparation of 4-(3-(2,6-bis(benzyloxy)pyridin-3-yl)-1-methyl-1H-indazol-6-yl)piperidin-4-ol

To a stirred solution of tert-butyl 4-(3-(2,6-bis(benzyloxy)pyridin-3-yl)-1-methyl-1H-indazol-6-yl)-4-hydroxypiperidine-1-carboxylate (2.0 g, 3.22 mmol) in DCM (20 mL) was added TFA (4.0 mL). The mixture was stirred at room temperature for 2 h. The reaction mixture was concentrated under reduced pressure to obtain crude product which was triturated with n-pentane (10.0 ml) and dried to afford 4-(3-(2,6-bis(benzyloxy)pyridin-3-yl)-1-methyl-1H-indazol-6-yl)piperidin-4-ol (1.5 g) as a yellow solid.

LC-MS (ESI): m/z=521.34 [M+H]⁺

### Preparation of tert-butyl 4-((4-(3-(2,6-bis(benzyloxy)pyridin-3-yl)-1-methyl-1H-indazol-6-yl)-4-hydroxypiperidin-1-yl)methyl)piperidine-1-carboxylate

To a stirred solution of 4-(3-(2,6-bis(benzyloxy)pyridin-3-yl)-1-methyl-1H-indazol-6-yl)piperidin-4-ol (1.5 g, 2.88 mmol) and tert-butyl 4-formylpiperidine-1-carboxylate (0.73 g, 3.45 mmol) in THF (15.00 mL) were added acetic acid (0.16 mL, 2.88 mmol), sodium acetate anhydrous (0.71 g, 8.64 mmol), and sodium triacetoxyborohydride (1.83 g, 8.64 mmol) at 0° C.

The mixture was stirred at room temperature for 12 h. The reaction mixture was quenched with water (30.0 mL) and extracted with DCM (50.0 mL). The separated organic layer was washed with brine (20.0 mL), dried over anhydrous Na₂SO₄, filtered, and dried under vacuum to get a crude product which was purified by flash column chromatography (80% ethyl acetate in petroleum ether) to afford tert-butyl 4-((4-(3-(2,6-bis(benzyloxy)pyridin-3-yl)-1-methyl-1H-indazol-6-yl)-4-hydroxypiperidin-1-yl)methyl)piperidine-1-carboxylate (1.4 g) as a yellow solid.

LC-MS (ESI): m/z=628.43 [M+H]⁺

### Preparation of tert-butyl 4-((4-(3-(2,6-dioxopiperidin-3-yl)-1-methyl-1H-indazol-6-yl)-4-hydroxypiperidin-1-yl)methyl)piperidine-1-carboxylate

To the stirred solution of tert-butyl 4-((4-(3-(2,6-bis(benzyloxy)pyridin-3-yl)-1-methyl-1H-indazol-6-yl)-4-hydroxypiperidin-1-yl)methyl)piperidine-1-carboxylate (1.4 g, 1.95 mmol) in THF (28.0 mL) was added 20% palladium hydroxide on carbon (1.4 g, 100% w/w). The mixture was stirred under H₂ (80 psi) pressure at room temperature for 6 h. The reaction mixture was filtered through a celite bed and washed with ethyl acetate (10 mL). The filtrate was concentrated under vacuum to get a crude product which was purified by flash column chromatography (100% ethyl acetate) to afford tert-butyl 4-((4-(3-(2,6-dioxopiperidin-3-yl)-1-methyl-1H-indazol-6-yl)-4-hydroxypiperidin-1-yl)methyl)piperidine-1-carboxylate (0.93 g) as an off white solid.

LC-MS (ESI): m/z=540.50 [M+H]⁺

### Preparation of 3-(6-(4-hydroxy-1-(piperidin-4-ylmethyl)piperidin-4-yl)-1-methyl-1H-indazol-3-yl)piperidine-2,6-dione

To a stirred solution of tert-butyl 4-((4-(3-(2,6-dioxopiperidin-3-yl)-1-methyl-1H-indazol-6-yl)-4-hydroxypiperidin-1-yl)methyl)piperidine-1-carboxylate (0.93 g, 1.72 mmol) in DCM (10 mL) was added TFA (5 mL) at 0° C. The mixture was stirred at room temperature for 2 h. The reaction mixture was concentrated under reduced pressure to obtain a crude product which was triturated with n-pentane (10.0 mL) to afford 3-(6-(4-hydroxy-1-(piperidin-4-ylmethyl)piperidin-4-yl)-1-methyl-1H-indazol-3-yl)piperidine-2,6-dione (0.7 g) as an off-white solid.

LC-MS (ESI): m/z=440.50 [M+H]⁺

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-hydroxypiperidin-4-yl)-1-methyl-1H-indazol-3-yl)piperidine-2,6-dione

To a stirred solution of 3-(6-(4-hydroxy-1-(piperidin-4-ylmethyl)piperidin-4-yl)-1-methyl-1H-indazol-3-yl)piperidine-2,6-dione (0.2 g, 0.45 mmol) and (S)-2-cyclopropyl-10-((2,5-dichloropyrimidin-4-yl)amino)-3,3-difluoro-7-methyl-1,2,3,4-tetrahydro [1,4]oxazepino [2,3-c]quinolin-6(7H)-one (0.08 g, 0.18 mmol) in DMSO (4.0 mL) was added DIPEA (0.24 mL, 1.82 mmol). The mixture was heated at 100° C. and stirred for 3 h. The reaction mixture was quenched with ice-cold water and filtered to get a crude product which was triturated with n-pentane (10.0 mL) to afford 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-hydroxypiperidin-4-yl)-1-methyl-1H-indazol-3-yl)piperidine-2,6-dione (0.15 g) as an off-white solid.

LC-MS (ESI): m/z=871.36 [M+H]⁺

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-fluoropiperidin-4-yl)-1-methyl-1H-indazol-3-yl)piperidine-2,6-dione

To a stirred solution of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-hydroxypiperidin-4-yl)-1-methyl-1H-indazol-3-yl)piperidine-2,6-dione (0.300 g, 0.344 mmol) in DCM (3.0 mL) at 0° C. was added diethylaminosulfur trifluoride (DAST) (0.083 g, 0.516 mmol). The mixture was stirred at room temperature for 1 h under N₂ atmosphere. The reaction mixture was concentrated under reduced pressure to obtain a crude product which was purified by prep-HPLC to afford 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-fluoropiperidin-4-yl)-1-methyl-1H-indazol-3-yl)piperidine-2,6-dione as an off white solid.

LC-MS (ESI): m/z=873.37 [M+H]⁺

¹H NMR 400 MHz, DMSO-d6: δ 10.92 (s, 1H), 8.99 (s, 1H), 8.90 (s, 1H), 8.16 (s, 1H), 8.07 (s, 1H), 7.77 (dd, J=7.20, 14.80 Hz, 2H), 7.63 (s, 1H), 7.43 (d, J=9.20 Hz, 1H), 7.08-7.11 (m, 1H), 6.26 (s, 1H), 4.50-4.38 (m, 5H), 4.03 (s, 3H), 3.57 (s, 5H), 3.30-3.20 (m, 3H), 3.13 (s, 2H), 2.90-2.80 (m, 2H), 2.70 (s, 2H), 2.62-2.50 (m, 3H), 2.40-2.20 (m, 2H), 2.20-2.00 (m, 2H), 1.74 (s, 2H), 1.25-1.20 (m, 1H), 0.80-0.75 (m, 1H), 0.59-0.50 (m, 2H), 0.39-0.35 (m, 1H).

### Example 169: 3-[6-[4-[[1-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-4-piperidyl]methyl]piperazin-1-yl]-1-methyl-indazol-3-yl]piperidine-2,6-dione (Compound 536a)

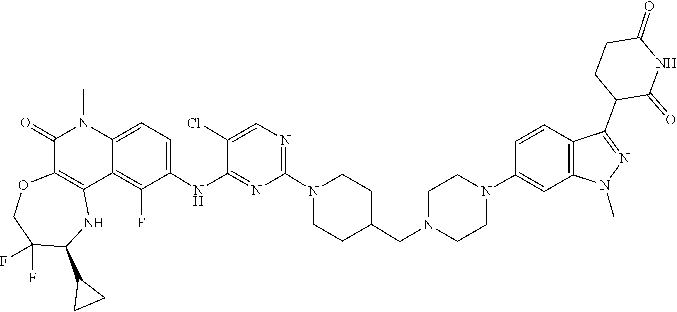 [SMILES: Cn1nc(C2CCC(=O)NC2=O)c2ccc(N3CCN(CC4CCN(c5ncc(Cl)c(Nc6ccc7c(c6F)c6c(c(=O)n7C)OCC(F)(F)[C@H](C7CC7)N6)n5)CC4)CC3)cc21]

### Preparation of (2S)-2-cyclopropyl-10-[(2,5-dichloropyrimidin-4-yl)amino]-3,3,11-trifluoro-7-methyl-2,4-dihydro-1H-[1,4]oxazepino[2,3-c]quinolin-6-one

To a solution of (2S)-10-amino-2-cyclopropyl-3,3,11-trifluoro-7-methyl-2,4-dihydro-1H-[1,4]oxazepino[2,3-c]quinolin-6-one (0.2 g, 532.24 mol, HCl) in DMF (3 mL) was added DIEA (206.36 mg, 1.60 mmol, 278.12 μL) and 2,4,5-trichloropyrimidine (126.91 mg, 691.91 mol). The mixture was stirred at 70° C. for 12 hr. The reaction mixture was diluted with H₂O (5 mL) and extracted with ethyl acetate (10 mL×3). The combined organic layers were washed with NaCl (10 mL×2), dried over Na₂SO₄, filtered, and concentrated under reduced pressure to give a residue. The residue was purified by column chromatography (petroleum ether/ethyl acetate=1/1 to 1/3) to afford (2S)-2-cyclopropyl-10-[(2,5-dichloropyrimidin-4-yl)amino]-3,3,11-trifluoro-7-methyl-2,4-dihydro-1H-[1,4]oxazepino[2,3-c]quinolin-6-one (0.2 g, 352.27 mol) as a yellow solid.

LC-MS (ESI): m/z=486.1 [M+H]⁺

### Preparation of tert-butyl 4-[[4-[3-(2,6-dioxo-3-piperidyl)-1-methyl-indazol-6-yl]piperazin-1-yl]methyl]piperidine-1-carboxylate

To a solution of 3-(1-methyl-6-piperazin-1-yl-indazol-3-yl)piperidine-2,6-dione (1 g, 2.75 mmol, HCl) in DCM (1.2 mL) was added AcOH (16.51 mg, 274.85 mol, 15.73 μL) and tert-butyl 4-formylpiperidine-1-carboxylate (879.26 mg, 4.12 mmol). The mixture was stirred at 25° C. for 1 hour. To the mixture was added NaBH(OAc)₃ (1.17 g, 5.50 mmol). The resulting mixture was stirred at 25° C. for 12 hr. The reaction mixture was diluted with H₂O (10 mL) and extracted with DCM 60 mL (20 mL×3). The combined organic layers were dried over Na₂SO₄, filtered, and concentrated under reduced pressure to give a residue. The residue was purified by column chromatography DCM/MeOH=15/1 to 10/1) to afford tert-butyl 4-[[4-[3-(2,6-dioxo-3-piperidyl)-1-methyl-indazol-6-yl]piperazin-1-yl]methyl]piperidine-1-carboxylate (1 g, 1.71 mmol) as a yellow oil.

LC-MS (ESI): m/z=525.3 [M+H]⁺

### Preparation of 3-[1-methyl-6-[4-(4-piperidylmethyl)piperazin-1-yl]indazol-3-yl]piperidine-2,6-dione

To a solution of tert-butyl 4-[[4-[3-(2,6-dioxo-3-piperidyl)-1-methyl-indazol-6-yl]piperazin-1-yl]methyl]piperidine-1-carboxylate (0.3 g, 571.81 mol) in dioxane (0.5 mL) was added HCl/dioxane (4 M, 142.95 μL). The mixture was stirred at 25° C. for 0.5 hr. The reaction mixture was filtered, and the filter cake was washed with DCM to afford 3-[1-methyl-6-[4-(4-piperidylmethyl)piperazin-1-yl]indazol-3-yl]piperidine-2,6-dione (0.15 g, 353.33 mol) as a white solid.

LC-MS (ESI): m/z=425.3 [M+H]⁺

### Preparation of 3-[6-[4-[[1-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-4-piperidyl]methyl]piperazin-1-yl]-1-methyl-indazol-3-yl]piperidine-2,6-dione

To a solution of 3-[1-methyl-6-[4-(4-piperidylmethyl)piperazin-1-yl]indazol-3-yl]piperidine-2,6-dione (0.05 g, 117.78 mol) and (2S)-2-cyclopropyl-10-[(2,5-dichloropyrimidin-4-yl)amino]-3,3,11-trifluoro-7-methyl-2,4-dihydro-1H-[1,4]oxazepino[2,3-c]quinolin-6-one (57.27 mg, 117.78 mol) in DMSO (1 mL) was added Et₃N (47.67 mg, 471.10 mol, 65.57 μL). The mixture was stirred at 100° C. for 12 hours. The crude product was purified by reverse-phase HPLC to afford 3-[6-[4-[[1-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-4-piperidyl]methyl]piperazin-1-yl]-1-methyl-indazol-3-yl]piperidine-2,6-dione (9.64 mg, 10.76 mol) as a yellow solid.

¹H NMR (400 MHz, DMSO-d6) δ=10.84 (s, 1H), 8.75 (s, 1H), 8.21 (s, 0.36H), 8.02 (s, 1H), 7.70-7.58 (m, 1H), 7.49 (d, J=9.2 Hz, 1H), 7.39-7.29 (m, 1H), 6.90 (d, J=8.4 Hz, 1H), 6.82 (s, 1H), 5.85-5.62 (m, 1H), 4.54-4.18 (m, 6H), 3.88 (s, 3H), 3.57 (s, 3H), 3.20 (d, J=1.6 Hz, 5H), 2.78-2.70 (m, 2H), 2.20-2.11 (m, 4H), 1.79-1.64 (m, 4H), 1.39-1.20 (m, 2H), 1.06-0.89 (m, 3H), 0.76-0.64 (m, 1H), 0.60-0.48 (m, 3H), 0.44-0.31 (m, 1H)

LC-MS (ESI): m/z=438.0 [M+H]⁺

### Example 170: 3-[6-[(2S, 4R)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione (Compound 538a)

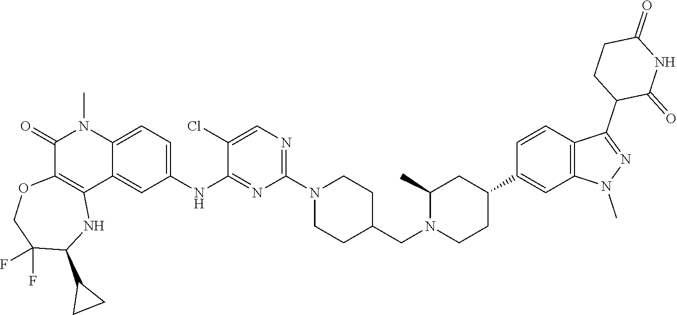 [SMILES: C[C@H]1C[C@H](c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1]

### Preparation of tert-butyl (2S)-4-hydroxy-2-methyl-piperidine-1-carboxylate

To a solution of tert-butyl (2S)-2-methyl-4-oxo-piperidine-1-carboxylate (5 g, 23.4 mmol) in MeOH (50 mL) was added dropwise NaBH₄ (1.33 g, 35.2 mmol) at 0° C. The resulting mixture was stirred at 25° C. for 3 hr. The reaction mixture was quenched by addition NH₄Cl (40 mL) and extracted with ethyl acetate 120 mL (40 mL×3), dried over Na₂SO₄, filtered and concentrated under reduced pressure to give a residue. The residue was purified by flash silica gel chromatography (0-30% ethyl acetate/petroleum ether) to give tert-butyl (2S)-4-hydroxy-2-methyl-piperidine-1-carboxylate (5 g, 23.2 mmol) as a white solid.

LC-MS (ESI): m/z=260.4 [M+H]⁺

¹H NMR (400 MHz, CDCl₃) δ=4.34-4.23 (m, 1H), 4.20-4.15 (m, 1H), 3.87-3.78 (m, 1H), 3.26 (dt, J=3.6, 12.8 Hz, 1H), 1.86-1.80 (m, 1H), 1.75-1.61 (m, 3H), 1.47 (s, 9H), 1.33 (d, J=7.2 Hz, 3H).

### Preparation of tert-butyl (2S)-4-iodo-2-methyl-piperidine-1-carboxylate

To a solution of 12 (3.68 g, 14.5 mmol, 2.92 mL) in DCE (30.0 mL) was added dropwise PPh₃ (4.12 g, 15.7 mmol) at 0° C. The mixture was stirred at 0° C. for 1.5 h, and then imidazole (1.23 g, 18.1 mmol) and tert-butyl (2S)-4-hydroxy-2-methyl-piperidine-1-carboxylate (2.60 g, 12.1 mmol) were added to the mixture. The resulting mixture was stirred at 25° C. for 12 hours. The reaction mixture was quenched by addition of NH₄Cl (10 mL), extracted with DCM (90 mL), dried over Na₂SO₄, filtered, and concentrated under reduced pressure to give a residue. The residue was purified by flash silica gel chromatography (0-10% ethyl acetate/petroleum ether) to give tert-butyl (2S)-4-iodo-2-methyl-piperidine-1-carboxylate (2.40 g, 6.86 mmol) as a white solid.

LC-MS (ESI): m/z=269.9 [M+H]⁺

¹H NMR (400 MHz, methanol-d4) δ=4.47 (td, J=4.4, 15.6 Hz, 1H), 4.33-4.24 (m, 1H), 4.16 (t, J=3.6, 6.8 Hz, 1H), 3.98-3.86 (m, 1H), 3.78-3.59 (m, 1H), 2.27-2.20 (m, 1H), 2.16-2.06 (m, 1H), 2.03-1.94 (m, 1H), 1.91-1.78 (m, 1H), 1.37-1.34 (m, 9H), 1.07 (d, J=7.2 Hz, 3H)

### Preparation of tert-butyl (2S, 4R)-4-(3-(2,6-bis(benzyloxy)pyridin-3-yl)-1-methyl-1H-indazol-6-yl)-2-methylpiperidine-1-carboxylate

To a 15 mL vial equipped with a stir bar was added 6-bromo-3-(2,6-dibenzyloxy-3-pyridyl)-1-methyl-indazole (2 g, 4 mmol), tert-butyl (2S)-4-iodo-2-methyl-piperidine-1-carboxylate (2.60 g, 7.99 mmol), TTMSS (994 mg, 4 mmol, 1.23 mL), Ir[dF(CF₃)ppy]₂(dtbpy)(PF₆) (44.9 mg, 40 mol), NiCl₂·dtbbpy (23.9 mg, 60 mol), and Na₂CO₃ (848 mg, 7.99 mmol) in DME (20 mL). The vial was sealed and placed under nitrogen. The reaction was stirred and irradiated with a 10 W blue LED lamp (3 cm away), with cooling water to keep the reaction temperature at 25° C. for 14 hours. The reaction mixture was extracted with ethyl acetate (90 mL). The combined organic layers were washed with aqueous NaCl (90 mL), dried over Na₂SO₄, filtered, and concentrated under reduced pressure to give a residue. The residue was purified by flash silica gel chromatography (0-30% ethyl acetate/petroleum ether) to give tert-butyl (2S, 4R)-4-(3-(2,6-bis(benzyloxy)pyridin-3-yl)-1-methyl-1H-indazol-6-yl)-2-methylpiperidine-1-carboxylate (1.40 g, 2.19 mmol) as a white solid.

LC-MS (ESI): m/z=619.3 [M+H]⁺

¹H NMR (400 MHz, methanol-d4) δ=7.86 (d, J=8.0 Hz, 1H), 7.58 (d, J=8.4 Hz, 1H), 7.46-7.40 (m, 2H), 7.38-7.33 (m, 3H), 7.33-7.21 (m, 6H), 6.96 (s, 1H), 6.53 (d, J=8.0 Hz, 1H), 5.42 (d, J=6.0 Hz, 4H), 4.63-4.46 (m, 1H), 4.05 (s, 3H), 3.18-3.03 (m, 2H), 1.96-1.85 (m, 2H), 1.81 (s, 1H), 1.66 (dq, J=4.0, 12.8 Hz, 2H), 1.49 (s, 8H), 1.30 (d, J=6.8 Hz, 3H)

### Preparation of tert-butyl (2S, 4R)-4-[3-(2,6-dioxo-3-piperidyl)-1-methyl-indazol-6-yl]-2-methyl-piperidine-1-carboxylate

To a solution of tert-butyl (2S, 4R)-4-(3-(2,6-bis(benzyloxy)pyridin-3-yl)-1-methyl-1H-indazol-6-yl)-2-methylpiperidine-1-carboxylate (300 mg, 485 mol, 1 eq) in CF₃CH₂OH (3 mL) was added Pd/C (516 mg, 485 mol, 10% purity) and Pd(OH)₂ (341 mg, 485 mol, 20% purity). The mixture was stirred at 25° C. for 12 hours under H₂ atmosphere. The reaction mixture was filtered and concentrated under reduced pressure to give tert-butyl (2S, 4R)-4-[3-(2,6-dioxo-3-piperidyl)-1-methyl-indazol-6-yl]-2-methyl-piperidine-1-carboxylate (0.2 g, crude) as a white solid.

### Preparation of 3-[1-methyl-6-[(2S, 4R)-2-methyl-4-piperidyl]indazol-3-yl]piperidine-2,6-dione

A solution of tert-butyl (2S, 4R)-4-[3-(2,6-dioxo-3-piperidyl)-1-methyl-indazol-6-yl]-2-methyl-piperidine-1-carboxylate (200 mg, 454 mol) in HCl/dioxane (1 mL) was stirred at 25° C. for 1 hour. The reaction mixture was filtered and concentrated under reduced pressure to give 3-[1-methyl-6-[(2S, 4R)-2-methyl-4-piperidyl]indazol-3-yl]piperidine-2,6-dione (0.2 g, crude) as a white solid.

### Preparation of tert-butyl 4-[[(2S, 4R)-4-[3-(2,6-dioxo-3-piperidyl)-1-methyl-indazol-6-yl]-2-methyl-1-piperidyl]methyl]piperidine-1-carboxylate

To a solution of 3-[1-methyl-6-[(2S, 4R)-2-methyl-4-piperidyl]indazol-3-yl]piperidine-2,6-dione (200 mg, 588 mol) in DCM (2 mL) was added dropwise AcOH (3.53 mg, 58.8 mol, 3.36 μL) and tert-butyl 4-formylpiperidine-1-carboxylate (188 mg, 882 mol) at 25° C. After addition, the mixture was stirred at this temperature for 30 min, and then NaBH(OAc)₃ (187 mg, 882 mol) was added dropwise at 25° C. The resulting mixture was stirred at 25° C. for 12 hours. The reaction mixture was quenched by addition of H₂O (10 mL) and extracted with DCM (60 mL), dried over Na₂SO₄, filtered and concentrated under reduced pressure to give a residue. The residue was purified by flash silica gel chromatography (0-30% ethyl acetate/petroleum ether) to give tert-butyl 4-[[(2S, 4R)-4-[3-(2,6-dioxo-3-piperidyl)-1-methyl-indazol-6-yl]-2-methyl-1-piperidyl]methyl]piperidine-1-carboxylate (110 mg, 186 mol) as a white solid.

LC-MS (ESI): m/z=538.3 [M+H]⁺

¹H NMR (400 MHz, Chloroform-d) 6=12.51-12.09 (m, 1H), 7.93 (s, 1H), 7.64 (d, J=7.6 Hz, 1H), 7.00 (s, 1H), 4.31 (dd, J=5.2, 7.2 Hz, 1H), 4.26-4.09 (m, 2H), 4.03 (s, 3H), 3.91 (dd, J=3.6, 4.8 Hz, 1H), 3.77 (t, J=6.4 Hz, 2H), 3.55-3.38 (m, 1H), 3.25-2.94 (m, 4H), 2.89-2.63 (m, 5H), 2.53 (s, 1H), 2.39 (d, J=5.2 Hz, 2H), 2.12 (s, 1H), 1.94-1.80 (m, 5H), 1.48 (s, 9H), 1.35-1.23 (m, 2H)

### Preparation of 3-[1-methyl-6-[(2S, 4R)-2-methyl-1-(4-piperidylmethyl)-4-piperidyl]indazol-3-yl]piperidine-2,6-dione

A solution of tert-butyl 4-[[(2S, 4R)-4-[3-(2,6-dioxo-3-piperidyl)-1-methyl-indazol-6-yl]-2-methyl-1-piperidyl]methyl]piperidine-1-carboxylate (110 mg, 205 mol) in DCM (0.5 mL) and TFA (0.2 mL) was stirred at 25° C. for 1 hour. The reaction mixture was filtered and concentrated under reduced pressure to give 3-[1-methyl-6-[(2S, 4R)-2-methyl-1-(4-piperidylmethyl)-4-piperidyl]indazol-3-yl]piperidine-2,6-dione (100 mg, crude) as a white solid.

### Preparation of 3-[6-[(2S, 4R)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione

To a solution of 3-[1-methyl-6-[(2S, 4R)-2-methyl-1-(4-piperidylmethyl)-4-piperidyl]indazol-3-yl]piperidine-2,6-dione (0.1 g, 229 mol) in DMSO (1 mL) was added TEA (92.5 mg, 914 mol, 127 μL) and (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (85.6 mg, 183 mol). The mixture was stirred at 100° C. for 2 hours. The reaction mixture was quenched by addition H₂O (4 mL) and extracted with ethyl acetate (60 mL). The combined organic layers were washed with aqueous NaCl (60 mL), dried over Na₂SO₄, filtered, and concentrated under reduced pressure to give a residue. The residue was purified by prep-HPLC to give 3-[6-[(2S, 4R)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione (29.2 mg, 32.6 mol) as a white solid.

LC-MS (ESI): m/z=869.6 [M+H]⁺

¹H NMR (400 MHz, DMSO-d₆) δ=10.88 (s, 1H), 8.78 (s, 1H), 8.20 (d, J=2.0 Hz, 1H), 8.03 (s, 1H), 7.74 (dd, J=2.0, 9.2 Hz, 1H), 7.60 (d, J=8.4 Hz, 1H), 7.51-7.38 (m, 2H), 7.04 (d, J=8.0 Hz, 1H), 6.16 (s, 1H), 4.51-4.41 (m, 3H), 4.41-4.28 (m, 2H), 3.99-3.94 (m, 3H), 3.57 (s, 3H), 3.12-3.07 (m, 1H), 3.04-2.88 (m, 2H), 2.79 (t, J=12.4 Hz, 2H), 2.69-2.59 (m, 3H), 2.39-2.32 (m, 1H), 2.26 (d, J=6.4 Hz, 2H), 2.21-2.13 (m, 1H), 2.02-1.90 (m, 1H), 1.86-1.54 (m, 7H), 1.41-1.27 (m, 1H), 1.04 (d, J=6.4 Hz, 5H), 0.77-0.66 (m, 1H), 0.53 (t, J=5.6 Hz, 2H), 0.36 (d, J=5.6 Hz, 1H).

### Example 171: 3-[6-[(2R, 4S)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione (Compound 538b)

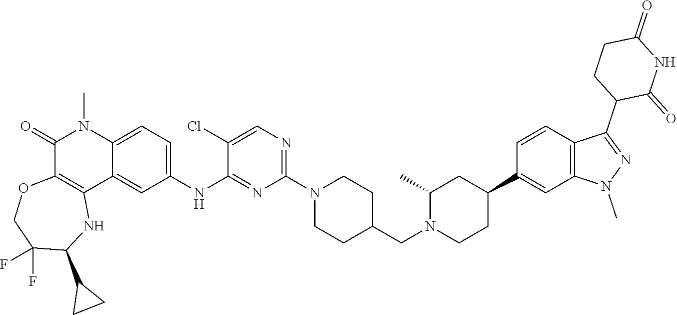 [SMILES: C[C@@H]1C[C@@H](c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1]

### Preparation of tert-butyl (2R)-4-hydroxy-2-methyl-piperidine-1-carboxylate

To a solution of tert-butyl (2R)-2-methyl-4-oxo-piperidine-1-carboxylate (10 g, 46.89 mmol) in MeOH (100 mL) was added NaBH₄ (2.66 g, 70.33 mmol). The mixture was stirred at 25° C. for 2 hours. The reaction mixture was quenched by addition NH₄Cl (300 mL) at 25° C., diluted with H₂O (500 mL), and extracted with ethyl acetate (500 mL). The combined organic layers were dried over Na₂SO₄, filtered, and concentrated under reduced pressure to give a residue. The residue was purified by column chromatography (petroleum ether/ethyl acetate=100/1 to 20/1) to afford tert-butyl (2R)-4-hydroxy-2-methyl-piperidine-1-carboxylate (5 g, 19.97 mmol) as a white solid.

LC-MS (ESI): m/z=142.1 [M+H]⁺.

¹H NMR (400 MHz, DMSO-d₆) δ=4.59 (d, J=2.8 Hz, 1H), 4.10 (dt, J=2.0, 6.8 Hz, 1H), 3.91 (m, J=3.2 Hz, 1H), 3.65-3.57 (m, 1H), 1.65 (dd, J=3.2, 6.4 Hz, 1H), 1.57-1.46 (m, 3H), 1.38 (s, 9H), 1.23 (d, J=7.2 Hz, 3H).

### Preparation of tert-butyl (2R)-4-iodo-2-methyl-piperidine-1-carboxylate

A solution of I₂ (7.07 g, 27.87 mmol, 5.61 mL) and PPh₃ (7.92 g, 30.19 mmol) in DCE (15 mL) at 0° C. was stirred for 90 min. To this mixture was added imidazole (2.37 g, 34.84 mmol), and tert-butyl (2R)-4-hydroxy-2-methyl-piperidine-1-carboxylate (5 g, 23.22 mmol) in DCE (15 mL) dropwise at 0° C. The resulting mixture was stirred at 25° C. for 12 hr. The reaction mixture was concentrated under reduced pressure to give a residue. The residue was purified by column chromatography (petroleum ether/ethyl acetate=100/1 to 5/1) to give tert-butyl (2R)-4-iodo-2-methyl-piperidine-1-carboxylate (2.68 g, 7.58 mmol) as a colorless oil.

LC-MS (ESI): m/z=269.9 [M+H]⁺.

¹H NMR (400 MHz, DMSO-d₆) δ=5.64 (s, 2H), 4.61 (tt, J=4.0, 12.4 Hz, 1H), 4.18-4.08 (m, 1H), 3.92 (s, 1H), 3.64 (d, J=13.6 Hz, 1H), 2.97-2.84 (m, 1H), 2.33-2.23 (m, 1H), 1.95 (dd, J=4.4, 7.2 Hz, 1H), 1.39 (d, J=7.6 Hz, 18H), 1.10 (dd, J=2.0, 6.8 Hz, 5H).

### Preparation of tert-butyl (2R, 4S)-4-(3-(2,6-bis(benzyloxy)pyridin-3-yl)-1-methyl-1H-indazol-6-yl)-2-methylpiperidine-1-carboxylate

A mixture of tert-butyl (2R)-4-iodo-2-methyl-piperidine-1-carboxylate (2 g, 6.15 mmol), 6-bromo-3-(2,6-dibenzyloxy-3-pyridyl)-1-methyl-indazole (3.08 g, 6.15 mmol), Na₂CO₃ (1.30 g, 12.30 mmol), TTMSS (1.53 g, 6.15 mmol, 1.90 mL), bis[3,5-difluoro-2-[5-(trifluoromethyl)-2-pyridyl]phenyl]iridium(1+); 4-tert-butyl-2-(4-tert-butyl-2-pyridyl)pyridine hexafluorophosphate (69.00 mg, 61.50 mol) and 4-tert-butyl-2-(4-tert butyl-2-pyridyl)pyridine dichloro nickel (36.72 mg, 92.26 mol) in DME (20 mL) was degassed and purged with N₂ three times. The mixture was stirred at 25° C. for 12 hours under N₂ atmosphere. The reaction mixture was concentrated under reduced pressure to give a residue. The residue was purified by column chromatography (petroleum ether/ethyl acetate=100/1 to 20/1) to give tert-butyl (2R, 4S)-4-(3-(2,6-bis(benzyloxy)pyridin-3-yl)-1-methyl-1H-indazol-6-yl)-2-methylpiperidine-1-carboxylate (1.2 g, 1.92 mmol) as a white solid.

LC-MS (ESI): m/z=619.5 [M+H]⁺.

¹H NMR (400 MHz, DMSO-d₆) δ=7.89 (d, J=8.0 Hz, 1H), 7.59 (d, J=8.4 Hz, 1H), 7.49-7.44 (m, 3H), 7.42-7.32 (m, 5H), 7.31-7.26 (m, 3H), 6.96-6.91 (m, 1H), 6.57 (d, J=8.0 Hz, 1H), 5.43 (d, J=14.4 Hz, 4H), 4.52-4.35 (m, 1H), 4.03 (s, 3H), 4.00-3.90 (m, 1H), 3.34 (s, 1H), 3.04 (br t, J=12.0 Hz, 1H), 1.80 (d, J=9.2 Hz, 2H), 1.72-1.66 (m, 1H), 1.57 (dq, J=4.4, 12.4 Hz, 1H), 1.43-1.43 (m, 1H), 1.43-1.43 (m, 1H), 1.42-1.42 (m, 1H), 1.43 (s, 6H), 1.21 (s, 3H).

### Preparation of tert-butyl (2R, 4S)-4-[3-(2,6-dioxo-3-piperidyl)-1-methyl-indazol-6-yl]-2-methyl-piperidine-1-carboxylate

A mixture of tert-butyl (2R, 4S)-4-(3-(2,6-bis(benzyloxy)pyridin-3-yl)-1-methyl-1H-indazol-6-yl)-2-methylpiperidine-1-carboxylate (555.56 mg, 808.06 mol), Pd/C (859.94 mg, 808.06 mol, 10% purity), and Pd(OH)₂ (567.42 mg, 808.06 mol, 20% purity) in CF₃CH₂OH (5 mL) was degassed and purged with H₂ three times. The mixture was stirred at 25° C. for 12 hours under H₂ atmosphere. The reaction mixture was filtered, and the filtrate was concentrated to give a residue.

The residue was purified by column chromatography (petroleum ether/ethyl acetate=100/1 to 25/1) to give tert-butyl (2R, 4S)-4-[3-(2,6-dioxo-3-piperidyl)-1-methyl-indazol-6-yl]-2-methyl-piperidine-1-carboxylate (350 mg, 715.04 mol) as a yellow solid.

LC-MS (ESI): m/z=441.2 [M+H]⁺.

¹H NMR (400 MHz, DMSO-d₆) δ=10.88 (s, 1H), 7.61 (d, J=8.4 Hz, 1H), 7.46 (s, 1H), 7.04 (d, J=8.4 Hz, 1H), 4.52-4.36 (m, 1H), 4.32 (dd, J=5.2, 9.6 Hz, 1H), 4.03 (q, J=7.2 Hz, 1H), 3.96 (s, 3H), 3.06 (m, 2H), 2.68-2.59 (m, 2H), 2.40-2.29 (m, 1H), 2.21-2.11 (m, 1H), 1.81 (d, J=11.4 Hz, 2H), 1.71 (s, 1H), 1.59 (dd, J=4.4, 12.4 Hz, 1H), 1.42 (s, 9H), 1.20 (d, J=8.8 Hz, 3H)

### Preparation of tert-butyl 4-[[(2R, 4S)-4-[3-(2,6-dioxo-3-piperidyl)-1-methyl-indazol-6-yl]-2-methyl-1-piperidyl]methyl]piperidine-1-carboxylate

To a solution of 3-[1-methyl-6-[(2R, 4S)-2-methyl-4-piperidyl]indazol-3-yl]piperidine-2,6-dione (200 mg, 587.51 mol) and tert-butyl 4-formylpiperidine-1-carboxylate (187.95 mg, 881.27 mol) in DCM (3 mL) was added AcOH (3.53 mg, 58.75 mol, 3.36 μL) and NaBH(OAc)₃ (186.78 mg, 881.27 mol). The mixture was stirred at 25° C. for 12 hours. The reaction mixture was concentrated under reduced pressure to give a residue. The residue was purified by column chromatography (DCM:MeOH=50:50) to give tert-butyl 4-[[(2R, 4S)-4-[3-(2,6-dioxo-3-piperidyl)-1-methyl-indazol-6-yl]-2-methyl-1-piperidyl]methyl]piperidine-1-carboxylate (200 mg, 360.80 mol) as a yellow solid.

LC-MS (ESI): m/z=538.3 [M+H]⁺.

¹H NMR (400 MHz, DMSO-d₆) δ=10.91-10.85 (m, 1H), 7.64 (d, J=8.0 Hz, 1H), 7.44 (s, 1H), 7.07 (d, J=8.0 Hz, 1H), 4.34 (dd, J=5.2, 9.6 Hz, 1H), 4.37-4.30 (m, 1H), 3.97 (s, 3H), 3.92 (d, J=11.6 Hz, 4H), 3.82 (d, J=13.6 Hz, 1H), 3.63-3.56 (m, 1H), 3.23 (t, J=5.4 Hz, 3H), 2.75-2.59 (m, 5H), 2.41-2.30 (m, 2H), 1.65-1.56 (m, 3H), 1.54-1.46 (m, 2H), 1.41-1.34 (s, 9H), 1.25-1.22 (m, 1H), 1.01-0.91 (m, 3H).

### Preparation of 3-[1-methyl-6-[(2R, 4S)-2-methyl-1-(4-piperidylmethyl)-4-piperidyl]indazol-3-yl]piperidine-2,6-dione

A solution of tert-butyl 4-[[(2R, 4S)-4-[3-(2,6-dioxo-3-piperidyl)-1-methyl-indazol-6-yl]-2-methyl-1-piperidyl]methyl]piperidine-1-carboxylate (200 mg, 371.96 mol) in TFA (1 mL)/DCM (2 mL) was stirred at 25° C. for 1 hour. The reaction mixture was concentrated under reduced pressure to give 3-[1-methyl-6-[(2R, 4S)-2-methyl-1-(4-piperidylmethyl)-4-piperidyl]indazol-3-yl]piperidine-2,6-dione which was used in the next step without further purification.

LC-MS (ESI): m/z=438.3 [M+H]⁺.

### Preparation of 3-[6-[(2R, 4S)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione

To a solution of 3-[1-methyl-6-[(2R, 4S)-2-methyl-1-(4-piperidylmethyl)-4-piperidyl]indazol-3-yl]piperidine-2,6-dione (100 mg, 228.53 mol) and (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (85.61 mg, 182.83 mol) in DMSO (2 mL) was added TEA (92.50 mg, 914.13 mol, 127.23 μL). The mixture was stirred at 100° C. for 12 hours. The reaction mixture was partitioned between water (100 mL) and ethyl acetate (300 mL). The organic phase was separated, and washed with brine (300 mL), dried over Na₂SO₄, filtered, and concentrated under reduced pressure to give a residue. The residue was purified by prep-HPLC to give 3-[6-[(2R, 4S)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione (10 mg, 10.93 mol) as a white solid.

LC-MS (ESI): m/z=869.5 [M+H]⁺.

¹H NMR (400 MHz, DMSO-d₆) δ=10.88 (s, 1H), 8.77 (s, 1H), 8.20 (d, J=2.0 Hz, 1H), 8.03 (s, 1H), 7.74 (dd, J=2.0, 9.2 Hz, 1H), 7.65-7.55 (m, 1H), 7.47-7.39 (m, 2H), 7.04-7.00 (m, 1H), 7.05-7.00 (m, 1H), 6.20-6.13 (m, 1H), 4.52-4.29 (m, 5H), 3.96 (s, 3H), 3.59-3.55 (m, 3H), 3.12-3.12 (m, 1H), 3.17-3.07 (m, 2H), 2.99-2.88 (m, 2H), 2.83-2.72 (m, 3H), 2.61 (d, J=5.6 Hz, 1H), 2.39-2.34 (m, 2H), 2.02-1.91 (m, 2H), 1.83-1.59 (m, 7H), 1.32 (s, 1H), 1.25-1.21 (m, 1H), 1.25-1.21 (m, 1H), 1.02-1.02 (m, 1H), 1.10-0.96 (m, 5H), 0.74-0.67 (m, 1H), 0.57-0.47 (m, 2H), 0.37-0.30 (m, 1H)

### Example 172: 3-[6-[1-[[1-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-4-piperidyl]methyl]-4-piperidyl]-1-methyl-indazol-3-yl]piperidine-2,6-dione (Compound 539a)

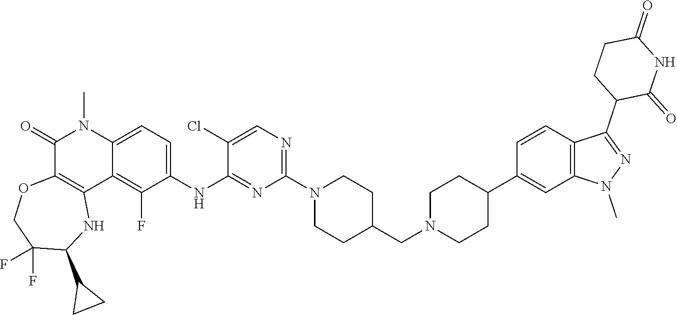 [SMILES: Cn1nc(C2CCC(=O)NC2=O)c2ccc(C3CCN(CC4CCN(c5ncc(Cl)c(Nc6ccc7c(c6F)c6c(c(=O)n7C)OCC(F)(F)[C@H](C7CC7)N6)n5)CC4)CC3)cc21]

### Preparation of 3-[6-[1-[[1-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-4-piperidyl]methyl]-4-piperidyl]-1-methyl-indazol-3-yl]piperidine-2,6-dione

To a solution of 3-[1-methyl-6-[1-(4-piperidylmethyl)-4-piperidyl]indazol-3-yl]piperidine-2,6-dione (100 mg, 236.10 mol) and (2S)-2-cyclopropyl-10-[(2,5-dichloropyrimidin-4-yl)amino]-3,3,11-trifluoro-7-methyl-2,4-dihydro-1H-[1,4]oxazepino[2,3-c]quinolin-6-one (91.85 mg, 188.88 mol) in DMSO (1 mL) was added TEA (95.56 mg, 944.40 mol, 131.45 μL). The mixture was stirred at 100° C. for 1 hour. The reaction mixture was filtered to give a residue. The residue was purified by prep-HPLC to afford 3-[6-[1-[[1-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-4-piperidyl]methyl]-4-piperidyl]-1-methyl-indazol-3-yl]piperidine-2,6-dione (59.44 mg, 66.58 mol) as a yellow solid.

LC-MS (ESI): m/z=6873.4 [M+H]⁺

¹H NMR (400 MHz, DMSO-d₄) δ=8.08 (s, 1H), 7.77 (t, J=8.8 Hz, 1H), 7.69 (d, J=8.4 Hz, 1H), 7.45 (d, J=9.6 Hz, 1H), 7.37 (s, 1H), 7.09 (d, J=8.4 Hz, 1H), 4.57-4.40 (m, 2H), 4.38-4.28 (m, 3H), 4.01 (s, 3H), 3.78-3.67 (m, 5H), 3.50-3.39 (m, 1H), 3.22-2.96 (m, 7H), 2.84-2.66 (m, 2H), 2.53-2.39 (m, 1H), 2.34-2.10 (m, 6H), 1.87 (t, J=10.4 Hz, 2H), 1.41-1.23 (m, 3H), 0.88-0.75 (m, 1H), 0.73-0.58 (m, 2H), 0.44-0.27 (m, 1H).

### Example 173: 3-(6-(4-((1r, 3r)-3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)cyclobutyl)piperazin-1-yl)-1-methyl-1H-indazol-3-yl)piperidine-2,6-dione (Compound 547a)

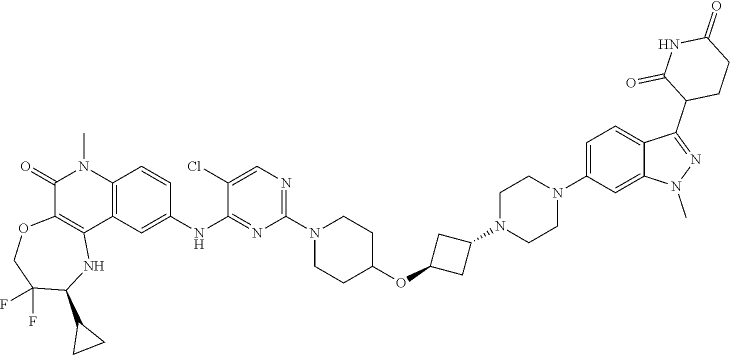 [SMILES: Cn1nc(C2CCC(=O)NC2=O)c2ccc(N3CCN([C@H]4C[C@H](OC5CCN(c6ncc(Cl)c(Nc7ccc8c(c7)c7c(c(=O)n8C)OCC(F)(F)[C@H](C8CC8)N7)n6)CC5)C4)CC3)cc21]

### Preparation of tert-butyl 4-((1r, 3r)-3-(piperazin-1-yl)cyclobutoxy)piperidine-1-carboxylate

To a stirred solution of benzyl 4-((1r, 3r)-3-((1-(tert-butoxycarbonyl)piperidin-4-yl)oxy) cyclobutyl)piperazine-1-carboxylate (1.7 g, 3.58 mmol) in THF (68 mL) was added 20% Pd(OH)₂ (2.01 g, 14.35 mmol). The mixture was stirred under hydrogen atmosphere (80 psi) at room temperature for 16 h. The reaction mixture was diluted with THF and filtered through a pad of celite and washed with excess 20% THF in DCM. The filtrate was collected and concentrated to afford tert-butyl 4-((1r, 3r)-3-(piperazin-1-yl)cyclobutoxy)piperidine-1-carboxylate (1.2 g) as a yellow semi-solid.

LC-MS (ESI): m/z=340.33[M+H]⁺

### Preparation of tert-butyl 4-((1r, 3r)-3-(4-(3-(2,6-bis(benzyloxy)pyridin-3-yl)-1-methyl-1H-indazol-6-yl)piperazin-1-yl)cyclobutoxy)piperidine-1-carboxylate

To a stirred solution of 3-(2,6-bis(benzyloxy)pyridin-3-yl)-6-bromo-1-methyl-1H-indazole (1.0 g, 1.99 mmol) and tert-butyl 4-((1r, 3r)-3-(piperazin-1-yl)cyclobutoxy)piperidine-1-carboxylate (1.14 g, 3.38 mmol) in 1,4-dioxane (10.0 mL) was added potassium tert-butoxide (0.66 g, 5.97 mmol). The mixture was purged with argon for 5 minutes followed by the addition of bis(tri-tert-butylphosphine)palladium (0) (0.10 g, 0.19 mmol). The resultant reaction mixture was heated at 100° C. for 16 h. The reaction was filtered through a celite pad and the filtrate was extracted with ethyl acetate (20 mL×2). The combined organic layer was washed with brine (10 mL), dried over sodium sulfate, filtered, and concentrated under reduced pressure to get a crude product which was purified by flash column chromatography (10% methanol in DCM) to afford tert-butyl 4-((1r, 3r)-3-(4-(3-(2,6-bis(benzyloxy)pyridin-3-yl)-1-methyl-1H-indazol-6-yl)piperazin-1-yl)cyclobutoxy)piperidine-1-carboxylate (1.0 g) as a brown semi-solid.

LC-MS (ESI): m/z=759.36 [M+H]⁺

### Preparation of tert-butyl 4-((1r, 3r)-3-(4-(3-(2,6-dioxopiperidin-3-yl)-1-methyl-1H-indazol-6-yl)piperazin-1-yl)cyclobutoxy)piperidine-1-carboxylate

To a stirred solution of tert-butyl 4-((1r, 3r)-3-(4-(3-(2,6-bis(benzyloxy)pyridin-3-yl)-1-methyl-1H-indazol-6-yl)piperazin-1-yl)cyclobutoxy)piperidine-1-carboxylate (1.0 g, 1.31 mmol) in THF (40 mL) was added 20% Pd(OH)₂ (0.74 g, 5.27 mmol). The mixture was stirred under hydrogen atmosphere (80 psi) at room temperature for 16 h. The reaction mixture was diluted with THF and filtered through a pad of celite and washed with an excess of 20% THF in DCM. The filtrate was collected and concentrated to afford tert-butyl 4-((1r, 3r)-3-(4-(3-(2,6-dioxopiperidin-3-yl)-1-methyl-1H-indazol-6-yl)piperazin-1-yl)cyclobutoxy)piperidine-1-carboxylate (0.75 g) as a yellow semi-solid.

LC-MS (ESI): m/z=581.77 [M+H]⁺

### Preparation of 3-(1-methyl-6-(4-((1r, 3r)-3-(piperidin-4-yloxy)cyclobutyl)piperazin-1-yl)-1H-indazol-3-yl)piperidine-2,6-dione

To a stirred solution of tert-butyl 4-((1r, 3r)-3-(4-(3-(2,6-dioxopiperidin-3-yl)-1-methyl-1H-indazol-6-yl)piperazin-1-yl)cyclobutoxy)piperidine-1-carboxylate (0.75 g, 1.29 mmol) in DCM (15 mL) was added TFA (0.69 g, 2.58 mmol). The mixture was stirred at room temperature for 2 h. The reaction mixture was concentrated under reduced pressure to get a crude product which was triturated with diethyl ether (5 mL). The solid was dried to afford 3-(1-methyl-6-(4-((1r, 3r)-3-(piperidin-4-yloxy)cyclobutyl)piperazin-1-yl)-1H-indazol-3-yl)piperidine-2,6-dione (0.6 g, crude) as a black semi-solid.

LC-MS (ESI): m/z=481.49 [M+H]⁺

### Preparation of 3-(6-(4-((1r, 3r)-3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)cyclobutyl)piperazin-1-yl)-1-methyl-1H-indazol-3-yl)piperidine-2,6-dione

To a stirred solution of 3-(1-methyl-6-(4-((1r, 3r)-3-(piperidin-4-yloxy)cyclobutyl)piperazin-1-yl)-1H-indazol-3-yl)piperidine-2,6-dione (0.3 g, 0.20 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.18 mmol) in DMSO (3.0 mL) was added N,N-diisopropylethylamine (0.89 mL, 4.99 mmol). The mixture was stirred at 100° C. for 6 h. The reaction mixture was poured with ice-cold water. The precipitated solid was filtered and dried under vacuum to get a crude product which was purified by prep-HPLC to afford 3-(6-(4-((1r, 3r)-3-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)oxy)cyclobutyl)piperazin-1-yl)-1-methyl-1H-indazol-3-yl)piperidine-2,6-dione (0.0378 g) as a brown solid.

LC-MS (ESI): m/z=912.38 [M+H]⁺

¹H NMR (400 MHz, DMSO-d6): δ 10.86 (s, 1H), 9.69 (br s, 1H), 8.94 (s, 1H), 8.21 (d, J=1.60 Hz, 1H), 8.06 (s, 1H), 7.69 (dd, J₁=1.60 Hz, J₂=9.20 Hz, 1H), 7.57 (d, J=9.60 Hz, 1H), 7.44 (d, J=9.20 Hz, 1H), 7.21-6.96 (m, 2H), 6.24 (s, 1H), 4.47-4.20 (m, 3H), 4.04-3.88 (m, 6H), 3.57 (s, 4H), 3.45-3.37 (m, 2H), 3.22-3.03 (m, 3H), 3.02-3.00 (m, 4H), 2.67-2.61 (m, 6H), 2.35-2.30 (m, 1H), 2.16-2.14 (m, 3H), 1.83-1.75 (m, 2H), 1.40-1.35 (m, 3H), 0.75-0.69 (m, 1H), 0.55-0.49 (m, 2H), 0.40-0.30 (m, 1H).

### Example 174: 3-(6-((S)-3-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)pyrrolidin-1-yl)-1-methyl-1H-indazol-3-yl)piperidine-2,6-dione (Compound 548a)

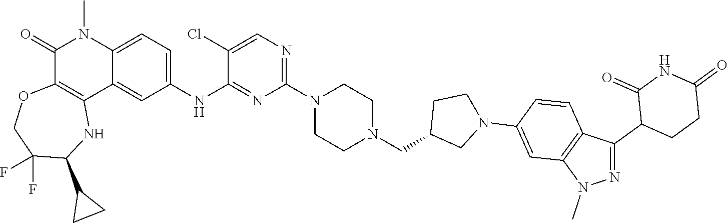 [SMILES: Cn1nc(C2CCC(=O)NC2=O)c2ccc(N3CC[C@@H](CN4CCN(c5ncc(Cl)c(Nc6ccc7c(c6)c6c(c(=O)n7C)OCC(F)(F)[C@H](C7CC7)N6)n5)CC4)C3)cc21]

### Preparation of 1-(tert-butyl) 3-methyl (R)-pyrrolidine-1,3-dicarboxylate

To a stirred solution of (R)-1-(tert-butoxycarbonyl)pyrrolidine-3-carboxylic acid (5.0 g, 23.22 mmol) in methanol (150 mL) was added conc. H₂SO₄ dropwise at 0° C. The mixture was stirred at 80° C. for 3 h. The reaction mixture was concentrated. The resulting residue was diluted with ethyl acetate (50 mL) and washed with sodium bicarbonate (20 mL) and brine (20 mL). The organic layer was dried over anhydrous Na₂SO₄, filtered, and concentrated to afford 1-(tert-butyl) 3-methyl (R)-pyrrolidine-1,3-dicarboxylate (5.0 g) as an off-white semi-solid.

LC-MS (ESI): m/z=252.24 [M+23]⁺

### Preparation of methyl (R)-pyrrolidine-3-carboxylate

To a stirred solution of 1-(tert-butyl) 3-methyl (R)-pyrrolidine-1,3-dicarboxylate (5.0 g, 21.80 mmol) in DCM (10.0 mL) was added 4M HCl in 1,4 dioxane (5.0 mL) at 0° C. The mixture was stirred at room temperature for 2 h. The reaction mixture was concentrated to obtain a crude product which was triturated with diethyl ether (30 mL) to afford methyl (R)-pyrrolidine-3-carboxylate (4.1 g) as an off-white semi-solid.

LC-MS (ESI): m/z=130.03 [M+H]⁺

### Preparation of methyl (R)-1-(3-(2,6-bis(benzyloxy)pyridin-3-yl)-1-methyl-1H-indazol-6-yl)pyrrolidine-3-carboxylate

A solution of methyl (R)-pyrrolidine-3-carboxylate (3.0 g, 23.2 mmol), 3-(2,6-bis(benzyloxy)pyridin-3-yl)-6-bromo-1-methyl-1H-indazole (5.8 g, 11.6 mmol), and cesium carbonate (22.6 g, 69.6 mmol) in dioxane (60 mL) was degassed with argon for 5 min. To this mixture was added Ruphos Pd G3 (0.9 g, 1.1 mmol). The mixture was heated at 100° C. and stirred for 16 h. The reaction mixture was filtered through a celite bed and concentrated under reduced pressure to obtain a crude product which was purified by column chromatography (50% ethyl acetate in petroleum ether) to afford methyl (R)-1-(3-(2,6-bis(benzyloxy)pyridin-3-yl)-1-methyl-1H-indazol-6-yl)pyrrolidine-3-carboxylate (1.3 g) as a pale-yellow semi-solid.

LC-MS (ESI): m/z=549.54 [M+H]⁺

### Preparation of (R)-1-(3-(2,6-bis(benzyloxy)pyridin-3-yl)-1-methyl-1H-indazol-6-yl)pyrrolidine-3-carboxylic acid

To a stirred solution of methyl (R)-1-(3-(2,6-bis(benzyloxy)pyridin-3-yl)-1-methyl-1H-indazol-6-yl)pyrrolidine-3-carboxylate (1.3 g, 2.36 mmol) in THF (13 mL) and H₂O (6.5 mL) was added LiOH·H₂O (0.5 g, 11.8 mmol) at 0° C. The mixture was stirred at room temperature for 2 h.

The reaction mixture was diluted with water (10 mL) and extracted with ethyl acetate (30 mL).

The organic layer was dried over sodium sulfate and concentrated under reduced pressure to afford (R)-1-(3-(2,6-bis(benzyloxy)pyridin-3-yl)-1-methyl-1H-indazol-6-yl)pyrrolidine-3-carboxylic acid (1.2 g) as an off white solid.

LC-MS (ESI): m/z=535.41 [M+H]⁺

### Preparation of tert-butyl (R)-4-(1-(3-(2,6-bis(benzyloxy)pyridin-3-yl)-1-methyl-1H-indazol-6-yl)pyrrolidine-3-carbonyl)piperazine-1-carboxylate

To a stirred solution of (R)-1-(3-(2,6-bis(benzyloxy)pyridin-3-yl)-1-methyl-1H-indazol-6-yl)pyrrolidine-3-carboxylic acid (1.10 g, 2.05 mmol) and tert-butyl piperazine-1-carboxylate (0.38 g, 2.05 mmol) in DMF (22.0 mL) was added N,N-diisopropylethylamine (1.07 mL, 6.15 mmol).

The mixture was stirred at room temperature for 5 min followed by the addition of HATU (1.16 g, 3.07 mmol) at 0° C. The mixture was stirred at room temperature for 2 h. The reaction mixture was poured into ice-cold water, filtered, and dried to afford tert-butyl (R)-4-(1-(3-(2,6-bis(benzyloxy)pyridin-3-yl)-1-methyl-1H-indazol-6-yl)pyrrolidine-3-carbonyl)piperazine-1-carboxylate (1.24 g) as an off white solid.

LC-MS (ESI): m/z=703.50 [M+H]⁺

### Preparation of tert-butyl (S)-4-((1-(3-(2,6-bis(benzyloxy)pyridin-3-yl)-1-methyl-1H-indazol-6-yl)pyrrolidin-3-yl)methyl)piperazine-1-carboxylate

To a stirred solution of tert-butyl (R)-4-(1-(3-(2,6-bis(benzyloxy)pyridin-3-yl)-1-methyl-1H-indazol-6-yl)pyrrolidine-3-carbonyl)piperazine-1-carboxylate (1.22 g, 1.73 mmol) in THF (24.4 mL) cooled to 0° C. was added borane dimethyl sulfide (neat) (12.2 mL). The mixture was stirred at room temperature for 16 h. The reaction mixture was quenched with methanol and evaporated under reduced pressure. The residue was dissolved in methanol and refluxed at 50° C. for 2 h. The reaction mixture was concentrated under reduced pressure to get crude product which was purified by column chromatography (70% ethyl acetate in petroleum ether) to afford tert-butyl (S)-4-((1-(3-(2,6-bis(benzyloxy)pyridin-3-yl)-1-methyl-1H-indazol-6-yl)pyrrolidin-3-yl)methyl)piperazine-1-carboxylate (0.9 g) as a brown semi-solid.

LC-MS (ESI): m/z=689.67[M+H]⁺

### Preparation of tert-butyl 4-(((3S)-1-(3-(2,6-dioxopiperidin-3-yl)-1-methyl-1H-indazol-6-yl)pyrrolidin-3-yl)methyl)piperazine-1-carboxylate

To a stirred solution of tert-butyl (S)-4-((1-(3-(2,6-bis(benzyloxy)pyridin-3-yl)-1-methyl-1H-indazol-6-yl)pyrrolidin-3-yl)methyl)piperazine-1-carboxylate (0.86 g, 1.24 mmol) in DMF (25.8 mL) and ACOH (0.86 mL) was added 20% Pd(OH)₂ (1.72 g). The mixture was stirred under hydrogen pressure (20 psi) at room temperature for 16 h. The reaction mixture was diluted with THF (100 mL), filtered through a celite bed, and washed with THF (200 mL). The filtrate was concentrated and dried to get tert-butyl 4-(((3S)-1-(3-(2,6-dioxopiperidin-3-yl)-1-methyl-1H-indazol-6-yl)pyrrolidin-3-yl)methyl)piperazine-1-carboxylate (0.6 g) as a light-brown semi-solid.

LC-MS (ESI): m/z=511.26 [M+H]⁺

### Preparation of 3-(1-methyl-6-((S)-3-(piperazin-1-ylmethyl)pyrrolidin-1-yl)-1H-indazol-3-yl)piperidine-2,6-dione

To a stirred solution of tert-butyl 4-(((3S)-1-(3-(2,6-dioxopiperidin-3-yl)-1-methyl-1H-indazol-6-yl)pyrrolidin-3-yl)methyl)piperazine-1-carboxylate (0.6 g, 1.17 mmol) in DCM (6.0 mL) was added TFA (3.0 mL) at 0° C. The mixture was stirred at room temperature for 2 h. The reaction mixture was concentrated under reduced pressure to get crude product which was triturated with diethyl ether (15 mL) to get 3-(1-methyl-6-((S)-3-(piperazin-1-ylmethyl)pyrrolidin-1-yl)-1H-indazol-3-yl)piperidine-2,6-dione (0.5 g) as a light-brown solid.

LC-MS (ESI): m/z=411.37 [M+H]⁺

### Preparation of 3-(6-((S)-3-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)pyrrolidin-1-yl)-1-methyl-1H-indazol-3-yl)piperidine-2,6-dione

To a stirred solution of 3-(1-methyl-6-((S)-3-(piperazin-1-ylmethyl)pyrrolidin-1-yl)-1H-indazol-3-yl)piperidine-2,6-dione (0.25 g, 0.60 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.08 g, 0.18 mmol) in DMSO (5.0 mL) was added N,N-diisopropylethylamine (0.53 mL, 3.04 mmol). The mixture was stirred at 100° C. for 3 h. The reaction mixture was poured into ice cold water (10 mL). The resulting solid was filtered and dried to get a crude product which was purified by prep-HPLC to afford 3-(6-((S)-3-((4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperazin-1-yl)methyl)pyrrolidin-1-yl)-1-methyl-1H-indazol-3-yl)piperidine-2,6-dione (0.06 g) as an off-white solid.

LC-MS (ESI): m/z=842.71 [M+H]⁺¹H NMR (400 MHz, DMSO-d6): δ 10.89 (s, 1H), 8.85 (s, 1H), 8.16 (s, 1H), 8.05 (s, 1H), 7.70-7.68 (m, 1H), 7.46-7.42 (m, 2H), 6.55-6.53 (m, 1H), 6.33 (s, 1H), 6.23 (s, 1H), 4.50-4.22 (m, 3H), 3.84 (s, 3H), 3.65-3.40 (m, 10H), 3.10-3.03 (br, 1H), 3.06-3.02 (m, 1H), 2.65 (br, 3H), 2.45-2.30 (m, 4H), 2.22-2.05 (m, 1H), 1.80-1.68 (m, 3H), 1.36 (br, 3H), 0.75-0.67 (m, 1H), 0.58-0.49 (m, 2H), 0.40-0.32 (m, 1H).

### Example 175: 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-(hydroxymethyl)piperazin-1-yl)methyl)piperidin-1-yl)-1-methyl-1H-indazol-3-yl)piperidine-2,6-dione (Compound 549a)

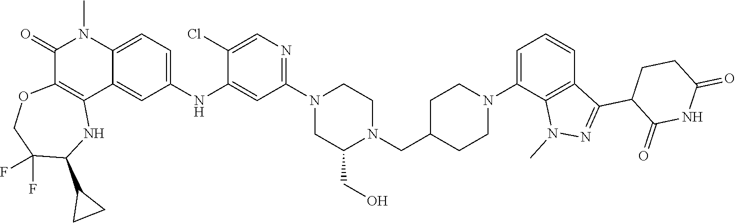 [SMILES: Cn1nc(C2CCC(=O)NC2=O)c2cccc(N3CCC(CN4CCN(c5cc(Nc6ccc7c(c6)c6c(c(=O)n7C)OCC(F)(F)[C@H](C7CC7)N6)c(Cl)cn5)C[C@H]4CO)CC3)c21]

### Preparation of 7-(4-(1,3-dioxolan-2-yl)piperidin-1-yl)-3-(2,6-bis(benzyloxy)pyridin-3-yl)-1-methyl-1H-indazole

To a stirred solution of 3-(2,6-bis(benzyloxy)pyridin-3-yl)-7-bromo-1-methyl-1H-indazole (3.50 g, 6.99 mmol) and 4-(1,3-dioxolan-2-yl)piperidine (2.74 g, 17.48 mmol) in 1,4-dioxane (30.0 mL) was added cesium carbonate (6.83 g, 20.97 mmol). The reaction mixture was degassed with argon for 15 minutes. To this mixture was added Pd PEPPSI iHeptCl (0.34 g, 0.34 mmol). The mixture was heated at 100° C. and stirred for 12 h. The reaction mixture was filtered through a pad of celite and washed with ethyl acetate (200 mL). The collected filtrate was concentrated under vacuum to obtain a crude product which was purified by flash chromatography using (12% of ethyl acetate in petroleum ether) to afford 7-(4-(1,3-dioxolan-2-yl)piperidin-1-yl)-3-(2,6-bis(benzyloxy)pyridin-3-yl)-1-methyl-1H-indazole (2.5 g) as a pale-yellow semi-solid.

LC-MS (ESI): m/z=577.56 [M+H]⁺

### Preparation of 3-(7-(4-(1,3-dioxolan-2-yl)piperidin-1-yl)-1-methyl-1H-indazol-3-yl)piperidine-2,6-dione

To a stirred solution of 7-(4-(1,3-dioxolan-2-yl)piperidin-1-yl)-3-(2,6-bis(benzyloxy)pyridin-3-yl)-1-methyl-1H-indazole (2.50 g, 4.33 mmol) in THF (75.0 mL) was added 20% Pd(OH)₂ (2.50 g). The mixture was stirred under H₂ atmosphere (80 psi) for 8 h at room temperature. The reaction mixture was diluted with THF (20 mL), washed with 30% THF:DCM (50 mL), and filtered through a celite bed. The collected filtrate was concentrated under vacuum to afford 3-(7-(4-(1,3-dioxolan-2-yl)piperidin-1-yl)-1-methyl-1H-indazol-3-yl)piperidine-2,6-dione (1.2 g) as a green semi-solid which was used in the next step without further purification.

LC-MS (ESI): m/z=399.40 [M+H]⁺

### Preparation of 1-(3-(2,6-dioxopiperidin-3-yl)-1-methyl-1H-indazol-7-yl)piperidine-4-carbaldehyde

A stirred solution of 3-(7-(4-(1,3-dioxolan-2-yl)piperidin-1-yl)-1-methyl-1H-indazol-3-yl)piperidine-2,6-dione (1.20 g, 3.01 mmol) in formic acid (24.0 mL) was heated at 80° C. for 2 h. The reaction mixture was concentrated and co-distilled with petroleum ether (3×40 mL) under reduced pressure to get a crude product which was triturated with diethyl ether to afford 1-(3-(2,6-dioxopiperidin-3-yl)-1-methyl-1H-indazol-7-yl)piperidine-4-carbaldehyde (1.0 g) as an off-white solid.

LC-MS (ESI): m/z=355.28 [M+H]⁺

### Preparation of 3-(7-(4-(((S)-4-(5-chloro-4-iodopyridin-2-yl)-2-(hydroxymethyl)piperazin-1-yl)methyl)piperidin-1-yl)-1-methyl-1H-indazol-3-yl)piperidine-2,6-dione

To a solution of 1-(3-(2,6-dioxopiperidin-3-yl)-1-methyl-1H-indazol-7-yl)piperidine-4-carbaldehyde (1.13 g, 3.18 mmol), (S)-(4-(5-chloro-4-iodopyridin-2-yl)piperazin-2-yl)methanol (1.12 g, 3.10 mmol), and sodium acetate (0.78 g, 9.56 mmol) in THF (23.0 mL) was added acetic acid (1.13 mL). The mixture was stirred at room temperature for 2 h. To this mixture was added NaBH(OAc)₃ (1.35 g, 6.37 mmol) at 0° C. The mixture was stirred at room temperature for 16 h.

The reaction mixture was quenched with ice-cold water (40 mL) and extracted into DCM (3×50 mL). The combined organic layers were washed with brine (30 mL), dried over anhydrous Na₂SO₄, filtered, and dried under vacuum to get a crude product which was purified by column chromatography (100% ethyl acetate with 1% TEA) to afford 3-(7-(4-(((S)-4-(5-chloro-4-iodopyridin-2-yl)-2-(hydroxymethyl)piperazin-1-yl)methyl)piperidin-1-yl)-1-methyl-1H-indazol-3-yl)piperidine-2,6-dione (0.80 g) as a brown semi-solid.

LC-MS (ESI): m/z=692.32 [M+H]⁺

### Preparation of 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-(hydroxymethyl)piperazin-1-yl)methyl)piperidin-1-yl)-1-methyl-1H-indazol-3-yl)piperidine-2,6-dione

To a solution of 3-(7-(4-(((S)-4-(5-chloro-4-iodopyridin-2-yl)-2-(hydroxymethyl)piperazin-1-yl)methyl)piperidin-1-yl)-1-methyl-1H-indazol-3-yl)piperidine-2,6-dione (0.35 g, 0.51 mmol) and (S)-10-amino-2-cyclopropyl-3,3-difluoro-7-methyl-1,2,3,4-tetrahydro-[1,4]oxazepino[2,3-c]quinolin-6(7H)-one (0.14 g, 0.45 mmol) in 1,4-dioxane (7.0 mL) was added cesium carbonate (0.49 g, 1.52 mmol). The mixture was degassed with argon for 15 minutes, followed by the addition of Ruphos (20 mg, 0.05 mmol) and RuPhos Pd-G3 (20 mg, 0.03 mmol). The resulting reaction mixture was heated at 100° C. and stirred for 6 h. The reaction mixture was filtered through a pad of celite and washed with ethyl acetate (20 mL). The collected filtrate was concentrated under vacuum to obtain a crude product which was purified by prep-HPLC to afford 3-(7-(4-(((S)-4-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-2-(hydroxymethyl)piperazin-1-yl)methyl)piperidin-1-yl)-1-methyl-1H-indazol-3-yl)piperidine-2,6-dione (57 mg) as an off white solid. ¹H NMR: (400 MHz, DMSO-d6): δ 10.87 (s, 1H), 8.06 (d, J=9.60 Hz, 2H), 7.93 (s, 1H), 7.48 (s, 2H), 7.36-7.35 (m, 2H), 7.01 (t, J=6.40 Hz, 2H), 6.39 (brs, 1H), 6.12 (s, 1H), 4.32-4.31 (m, 4H), 4.23 (s, 3H), 3.74 (d, J=10.00 Hz, 1H), 3.60-3.57 (m, 4H), 3.24-3.22 (m, 4H), 3.04-3.0 (m, 1H), 2.88-2.86 (m, 2H), 2.61-2.58 (m, 5H), 2.17-2.16 (m, 5H), 2.00-1.91 (m, 1H), 1.80-1.70 (m, 1H), 1.61 (brs, 1H), 1.29-1.24 (m, 4H), 0.81-0.61 (m, 1H), 0.51 (t, J=5.60 Hz, 2H), 0.33-0.31 (m, 1H).

LC-MS (ESI): m/z=885.81 [M+H]⁺

### Example 176: 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-5-fluoro-1-methyl-1H-indazol-3-yl)piperidine-2,6-dione (Compound 550a)

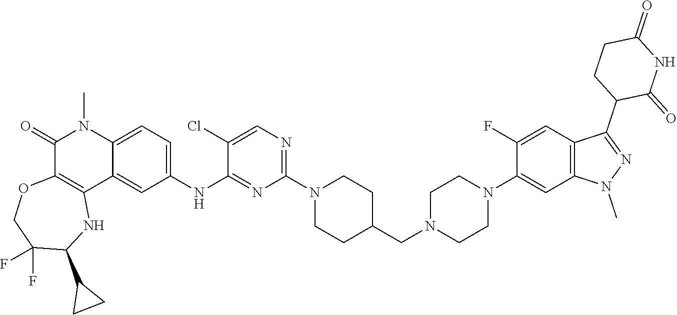 [SMILES: Cn1nc(C2CCC(=O)NC2=O)c2cc(F)c(N3CCN(CC4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-((1-(3-(2,6-bis(benzyloxy)pyridin-3-yl)-5-fluoro-1-methyl-1H-indazol-6-yl)piperidin-4-yl)methyl)piperazine-1-carboxylate

To a stirred solution of 3-(2,6-bis(benzyloxy)pyridin-3-yl)-6-bromo-5-fluoro-1-methyl-1H-indazole (1.50 g, 2.89 mmol) and tert-butyl 4-(piperazin-1-ylmethyl)piperidine-1-carboxylate (2.46 g, 8.68 mmol) in dioxane (30 mL) was added sodium tert-butoxide (0.83 g, 8.68 mmol). The reaction mixture was purged with N₂ for 15 min. To this mixture was added RuPhos Pd G3 (0.24 g, 0.29 mmol). The mixture was heated at 100° C. and stirred for 6 h. The reaction mixture was cooled to room temperature, diluted with ethyl acetate (300 mL), filtered through a celite bed, and washed with DCM (200 mL). The filtrate was concentrated to get a crude product which was purified by flash chromatography (5-10% of ethyl acetate in petroleum ether) to obtain tert-butyl 4-((1-(3-(2,6-bis(benzyloxy)pyridin-3-yl)-5-fluoro-1-methyl-1H-indazol-6-yl)piperidin-4-yl)methyl)piperazine-1-carboxylate as a pale-yellow semi-solid (0.65 g).

LC-MS (ESI): m/z=721.66 [M+H]⁺

### Preparation of tert-butyl 4-((4-(3-(2,6-dioxopiperidin-3-yl)-5-fluoro-1-methyl-1H-indazol-6-yl)piperazin-1-yl)methyl)piperidine-1-carboxylate

To a stirred solution of tert-butyl 4-((1-(3-(2,6-bis(benzyloxy)pyridin-3-yl)-5-fluoro-1-methyl-1H-indazol-6-yl)piperidin-4-yl)methyl)piperazine-1-carboxylate (0.65 g, 0.90 mmol) in THF (80 mL) was added 20% Pd(OH)₂ (0.65 g). The mixture was stirred under hydrogen atmosphere (60 psi) at room temperature for 3 h. The reaction mixture was diluted with DCM (200 mL), washed with 30% THF:DCM (300 mL), and filtered through a celite bed. The collected filtrate was concentrated under vacuum to obtain a crude product which was triturated with n-pentane to afford tert-butyl 4-((4-(3-(2,6-dioxopiperidin-3-yl)-5-fluoro-1-methyl-1H-indazol-6-yl)piperazin-1-yl)methyl)piperidine-1-carboxylate (0.46 g) as a pale-brown semi-solid.

LC-MS (ESI): m/z=543.56 [M+H]⁺

### Preparation of 3-(5-fluoro-1-methyl-6-(4-(piperidin-4-ylmethyl)piperazin-1-yl)-1H-indazol-3-yl)piperidine-2,6-dione

To a stirred solution of tert-butyl 4-((4-(3-(2,6-dioxopiperidin-3-yl)-5-fluoro-1-methyl-1H-indazol-6-yl)piperazin-1-yl)methyl)piperidine-1-carboxylate (0.46 g, 0.85 mmol) in DCM (5 mL) at 0° C., was added TFA (3.68 mL). The mixture was stirred at room temperature for 2 h. The reaction mixture was concentrated under reduced pressure to afford crude product which was triturated with n-pentane to afford 3-(5-fluoro-1-methyl-6-(4-(piperidin-4-ylmethyl)piperazin-1-yl)-1H-indazol-3-yl)piperidine-2,6-dione (0.45 g) as a pale brown semi-solid.

LC-MS (ESI): m/z=443.40 [M+H]⁺

### Preparation of 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-5-fluoro-1-methyl-1H-indazol-3-yl)piperidine-2,6-dione

To a stirred solution of 3-(5-fluoro-1-methyl-6-(4-(piperidin-4-ylmethyl)piperazin-1-yl)-1H-indazol-3-yl)piperidine-2,6-dione (0.45 g, 1.02 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.12 g, 0.25 mmol) in DMSO (5 mL) was added DIPEA (4.50 mL). The mixture was heated at 100° C. and stirred for 18 h. The reaction mixture was quenched with ice cold water (100 mL). The precipitated solid was filtered and dried under vacuum to afford a crude product which was purified by prep-HPLC to afford 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-5-fluoro-1-methyl-1H-indazol-3-yl)piperidine-2,6-dione (0.041 g) as an off white solid. ¹H NMR: (400 MHz, DMSO-d6: δ 10.86 (s, 1H), 8.78 (s, 1H), 8.17 (s, 1H), 8.03 (s, 1H), 7.74 (d, J=8.00 Hz, 1H), 7.44-7.46 (m, 2H), 7.09 (d, J=6.80 Hz, 1H), 6.19 (s, 1H), 4.45-4.25 (m, 4H), 3.94 (s, 3H), 3.56 (s, 3H), 3.20 (s, 1H), 3.06 (s, 4H), 2.80-2.78 (m, 2H), 2.64-2.60 (m, 6H), 2.36-2.35 (m, 1H), 2.20-2.12 (m, 3H), 1.90 (s, 1H), 1.71-1.75 (m, 2H), 1.36 (s, 2H), 1.04-1.02 (m, 2H), 0.73-0.71 (m, 1H), 0.53-0.51 (m, 2H), 0.35-0.33 (m, 1H).

LC-MS (ESI): m/z=874.41 [M+H]⁺

### Example 177: 3-[6-[4-[[3-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-3,9-diazaspiro[5.5]undecan-9-yl]methyl]-1-piperidyl]-1-methyl-indazol-3-yl]piperidine-2,6-dione (Compound 551a)

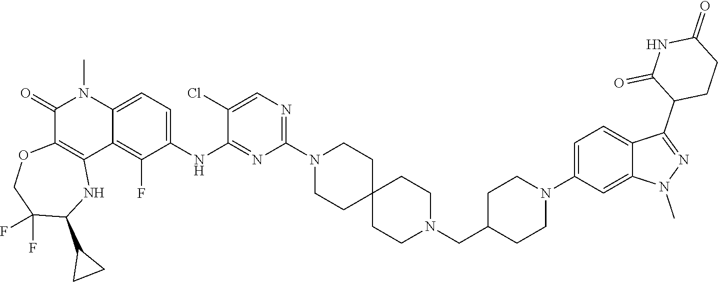 [SMILES: Cn1nc(C2CCC(=O)NC2=O)c2ccc(N3CCC(CN4CCC5(CC4)CCN(c4ncc(Cl)c(Nc6ccc7c(c6F)c6c(c(=O)n7C)OCC(F)(F)[C@H](C7CC7)N6)n4)CC5)CC3)cc21]

### Preparation of 3-[6-[4-[[3-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-3,9-diazaspiro[5.5]undecan-9-yl]methyl]-1-piperidyl]-1-methyl-indazol-3-yl]piperidine-2,6-dione

To a solution of 3-[6-[4-(3,9-diazaspiro[5.5]undecan-3-ylmethyl)-1-piperidyl]-1-methyl-indazol-3-yl]piperidine-2,6-dione (50 mg, 101.49 mol) in DMSO (1 mL) was added (2S)-2-cyclopropyl-10-[(2,5-dichloropyrimidin-4-yl)amino]-3,3,11-trifluoro-7-methyl-2,4-dihydro-1H-[1,4]oxazepino[2,3-c]quinolin-6-one (39.48 mg, 81.19 mol) and TEA (41.08 mg, 405.96 mol, 56.50 μL). The mixture was stirred at 100° C. for 12 h. The reaction mixture was filtered, and the resulting residue was purified by prep-HPLC to afford 3-[6-[4-[[3-[5-chloro-4-[[(2S)-2-cyclopropyl-3,3,11-trifluoro-7-methyl-6-oxo-2,4-dihydro-1H-[1,4]oxazepino[2,3-c]quinolin-10-yl]amino]pyrimidin-2-yl]-3,9-diazaspiro[5.5]undecan-9-yl]methyl]-1-piperidyl]-1-methyl-indazol-3-yl]piperidine-2,6-dione (25.31 mg, 25.77 mol) as a yellow solid.

LC-MS (ESI): m/z=942.4 [M+H]⁺

¹H NMR (400 MHz, DMSO-d₆) δ=10.84 (s, 1H), 8.75 (s, 1H), 8.20 (s, 1H), 8.02 (s, 1H), 7.63 (t, J=8.8 Hz, 1H), 7.46 (d, J=8.8 Hz, 1H), 7.34 (d, J=9.2 Hz, 1H), 6.88 (d, J=9.2 Hz, 1H), 6.81 (s, 1H), 5.86-5.68 (m, 1H), 4.59-4.35 (m, 2H), 4.28-4.25 (m, 1H), 3.87 (s, 3H), 3.76 (d, J=12.0 Hz, 2H), 3.58 (s, 3H), 3.49 (d, J=4.8 Hz, 4H), 2.76-2.66 (m, 2H), 2.65-2.57 (m, 2H), 2.30 (s, 5H), 2.15 (d, J=6.8 Hz, 3H), 2.07 (s, 1H), 1.77 (d, J=12.4 Hz, 2H), 1.72-1.61 (m, 1H), 1.43 (b s, 4H), 1.38-1.15 (m, 7H), 0.77-0.65 (m, 1H), 0.53 (t, J=6.0 Hz, 2H), 0.43-0.31 (m, 1H).

### Example 178: 3-(6-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-(hydroxymethyl)piperazin-1-yl)-1-methyl-1H-indazol-3-yl)piperidine-2,6-dione (Compound 560b)

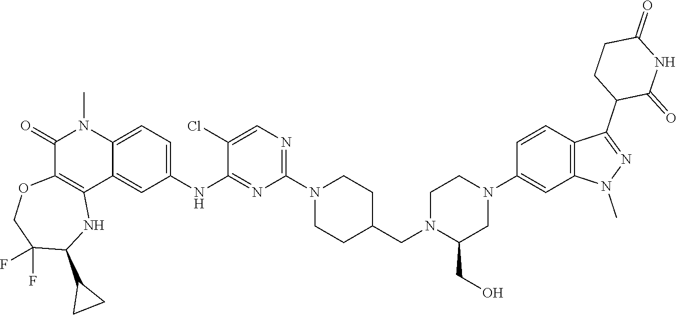 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)[C@H](CO)C3)cc21]

### Preparation of tert-butyl (S)-4-(3-(2,6-bis(benzyloxy)pyridin-3-yl)-1-methyl-1H-indazol-6-yl)-2-(hydroxymethyl)piperazine-1-carboxylate

To a stirred solution of 3-(2,6-bis(benzyloxy)pyridin-3-yl)-6-bromo-1-methyl-1H-indazole (2.50 g, 4.99 mmol) and tert-butyl (S)-2-(hydroxymethyl)piperazine-1-carboxylate (2.70 g, 12.49 mmol) in dioxane (50 mL) was added Cs₂CO₃ (4.88 g, 14.98 mmol). The reaction mixture was degassed with argon for 15 min. To this mixture was added Ruphos (0.23 g, 0.50 mmol) and Ruphos pd G3 (0.20 g, 0.25 mmol). The mixture was purged again for 2 min. The resultant reaction mixture was heated at 100° C. and stirred for 16 h. The reaction mixture was diluted with DCM (300 mL) and filtered through a pad of celite. The collected filtrate was concentrated under vacuum to obtain a crude product which was purified by flash chromatography (50-80% of ethyl acetate in petroleum ether) to afford tert-butyl (S)-4-(3-(2,6-bis(benzyloxy)pyridin-3-yl)-1-methyl-1H-indazol-6-yl)-2-(hydroxymethyl)piperazine-1-carboxylate (1.6 g) as a pale brown semi-solid.

LC-MS (ESI): m/z=636.87 [M+H]⁺

### Preparation of (S)-6-(benzyloxy)-5-(6-(3-(hydroxymethyl)piperazin-1-yl)-1-methyl-1H-indazol-3-yl)pyridin-2-ol

To stirred solution of a tert-butyl (S)-4-(3-(2,6-bis(benzyloxy)pyridin-3-yl)-1-methyl-1H-indazol-6-yl)-2-(hydroxymethyl)piperazine-1-carboxylate (1.60 g, 2.51 mmol) in DCM (32.0 mL) was added TFA (8.0 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL) to afford (S)-6-(benzyloxy)-5-(6-(3-(hydroxymethyl)piperazin-1-yl)-1-methyl-1H-indazol-3-yl)pyridin-2-ol (1.3 g) as a pale brown semi-solid.

LC-MS (ESI): m/z=446.18 [M+H]⁺

### Preparation of tert-butyl (S)-4-((4-(3-(2-(benzyloxy)-6-hydroxypyridin-3-yl)-1-methyl-1H-indazol-6-yl)-2-(hydroxymethyl)piperazin-1-yl)methyl)piperidine-1-carboxylate

To a stirred solution of (S)-6-(benzyloxy)-5-(6-(3-(hydroxymethyl)piperazin-1-yl)-1-methyl-1H-indazol-3-yl)pyridin-2-ol (1.3 g, 2.91 mmol.) in THF (26.0 mL) was added tert-butyl 4-formylpiperidine-1-carboxylate (0.93 g, 4.37 mmol), acetic acid (1.3 mL, 2.33 mmol), and sodium acetate (0.71 g, 8.75 mmol). The mixture was stirred for 2 h at room temperature, followed by the addition of sodium triacetoxyborohydride (1.85 g, 8.75 mmol) at 0° C. The mixture was stirred at room temperature for 16 h. The reaction mixture was quenched with NH₄Cl solution (30 mL) and extracted with ethyl acetate (30 mL×2). The combined organic layer was dried over anhydrous Na₂SO₄, filtered, and concentrated to obtain crude product which was purified by flash chromatography (50-100% of ethyl acetate in petroleum ether) to get tert-butyl (S)-4-((4-(3-(2-(benzyloxy)-6-hydroxypyridin-3-yl)-1-methyl-1H-indazol-6-yl)-2-(hydroxymethyl)piperazin-1-yl)methyl)piperidine-1-carboxylate (0.7 g) as a pale-brown semi-solid.

LC-MS (ESI): m/z=643.38 [M+H]⁺

### Preparation of tert-butyl 4-(((2S)-4-(3-(2,6-dioxopiperidin-3-yl)-1-methyl-1H-indazol-6-yl)-2-(hydroxymethyl)piperazin-1-yl)methyl)piperidine-1-carboxylate

To a stirred solution of tert-butyl (S)-4-((4-(3-(2-(benzyloxy)-6-hydroxypyridin-3-yl)-1-methyl-1H-indazol-6-yl)-2-(hydroxymethyl)piperazin-1-yl)methyl)piperidine-1-carboxylate (0.70 g, 1.08 mmol) in THF (140.0 mL) was added 20% palladium hydroxide (0.70 g, 5.00 mmol.). The mixture was stirred under H₂ (80 psi) pressure for 16 h at room temperature. The reaction mixture was diluted with THF (30 mL), filtered through a celite bed, and washed with THF:DCM (1:1, 20 mL).

The filtrate was concentrated and dried to obtain crude product which was triturated with diethyl ether to afford tert-butyl 4-(((2S)-4-(3-(2,6-dioxopiperidin-3-yl)-1-methyl-1H-indazol-6-yl)-2-(hydroxymethyl)piperazin-1-yl)methyl)piperidine-1-carboxylate (0.52 g) as a light brown semi-solid.

LC-MS (ESI): m/z=555.33 [M+H]⁺

### Preparation of 3-(6-((S)-3-(hydroxymethyl)-4-(piperidin-4-ylmethyl)piperazin-1-yl)-1-methyl-1H-indazol-3-yl)piperidine-2,6-dione

To stirred solution of a tert-butyl 4-(((2S)-4-(3-(2,6-dioxopiperidin-3-yl)-1-methyl-1H-indazol-6-yl)-2-(hydroxymethyl)piperazin-1-yl)methyl)piperidine-1-carboxylate (0.52 g, 0.3 mmol) in DCM (15.6 mL) was added TFA (2.6 mL) at 0° C. The mixture was stirred at room temperature for 2 h.

The reaction mixture was concentrated under reduced pressure and co-distilled with DCM (2×20 mL) to obtain a crude product which was triturated with diethyl ether (2×10 mL) and dried to afford 3-(6-((S)-3-(hydroxymethyl)-4-(piperidin-4-ylmethyl)piperazin-1-yl)-1-methyl-1H-indazol-3-yl)piperidine-2,6-dione (0.4 g) as a pale-yellow solid.

LC-MS (ESI): m/z=455.45 [M+H]⁺

### Preparation of 3-(6-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-(hydroxymethyl)piperazin-1-yl)-1-methyl-1H-indazol-3-yl)piperidine-2,6-dione

To a stirred solution of 3-(6-((S)-3-(hydroxymethyl)-4-(piperidin-4-ylmethyl)piperazin-1-yl)-1-methyl-1H-indazol-3-yl)piperidine-2,6-dione (0.2 g, 0.44 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.062 g, 0.132 mmol) in DMSO (3.0 mL) was added DIPEA (2.0 mL). The mixture was heated at 100° C. and stirred for 16 h. The reaction mixture was cooled to room temperature and poured into ice water. The precipitated solid was filtered and dried to obtain a crude product which was purified by prep-HPLC to afford 3-(6-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-3-(hydroxymethyl)piperazin-1-yl)-1-methyl-1H-indazol-3-yl)piperidine-2,6-dione (0.022 g) as an off-white solid.

¹H NMR (400 MHz, DMSO-d6): δ 10.85 (s, 1H), 8.76 (s, 1H), 8.17 (d, J=2.00 Hz, 1H), 8.03 (s, 1H), 7.75 (dd, J=10.80, Hz, 1H), 7.50 (d, J=9.20 Hz, 1H), 7.44 (d, J=9.20 Hz, 1H), 6.89 (d, J=12.00 Hz, 1H), 6.80 (s, 1H), 6.17 (s, 1H), 4.55-4.54 (m, 1H), 4.47-4.40 (m, 4H), 4.27-4.24 (m, 1H), 3.89 (s, 3H), 3.56 (s, 3H), 3.47-3.39 (m, 3H), 2.97-2.94 (m, 5H), 2.67-2.56 (m, 3H), 2.50-2.36 (m, 3H), 2.33-2.13 (m, 2H), 1.89 (s, 1H), 1.75 (m, 4H), 1.40 (br s, 1H), 1.12-0.98 (m, 2H), 0.75-0.65 (m, 1H), 0.54-0.51 (m, 2H), 0.36 (m, 1H).

LC-MS (ESI): m/z=886.50 [M+H]⁺

### Example 179: 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-7-fluoro-1-methyl-1H-indazol-3-yl)piperidine-2,6-dione (Compound 561a)

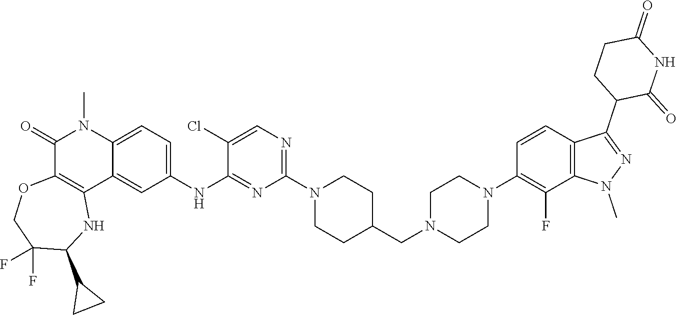 [SMILES: Cn1nc(C2CCC(=O)NC2=O)c2ccc(N3CCN(CC4CCN(c5ncc(Cl)c(Nc6ccc7c(c6)c6c(c(=O)n7C)OCC(F)(F)[C@H](C7CC7)N6)n5)CC4)CC3)c(F)c21]

### Preparation of benzyl 4-((1-(tert-butoxycarbonyl)piperidin-4-yl)methyl)piperazine-1-carboxylate

To a stirred solution of tert-butyl 4-formylpiperidine-1-carboxylate (5.0 g, 23.45 mmol) and benzyl piperazine-1-carboxylate (5.16 g, 23.45 mmol) in THF (100 ml) was added acetic acid (0.5 mL) at 0° C. The mixture was stirred for 2 h followed by the addition of NaBH(OAc)₃ (9.93 g, 46.90 mmol) at 0° C. The mixture was stirred at room temperature for 16 h. The reaction mixture was quenched with water (20 mL) and extracted with DCM (30 mL). The organic layer was separated and washed with NaHCO₃ solution (20 mL), dried over anhydrous Na₂SO₄, filtered, and concentrated to get a crude product, which was purified by flash column chromatography (100% ethyl acetate) to afford benzyl 4-((1-(tert-butoxycarbonyl)piperidin-4-yl)methyl)piperazine-1-carboxylate (6.0 g) as a brown gum

LC-MS (ESI): m/z=418.43 [M+H]⁺

### Preparation of tert-butyl 4-(piperazin-1-ylmethyl)piperidine-1-carboxylate

To a stirred solution of benzyl 4-((1-(tert-butoxycarbonyl)piperidin-4-yl)methyl)piperazine-1-carboxylate (4.5 g, 10.79 mmol) in THF (50 mL) at room temperature was added 10% Pd/C (100% w/w) (4.5 g) under inert atmosphere. The reaction mixture was kept under hydrogen pressure (90 psi) at room temperate and stirred for 16 h. The reaction mixture was filtered through celite and the filtrate was concentrated under reduced pressure to get tert-butyl 4-(piperazin-1-ylmethyl)piperidine-1-carboxylate (1.9 g) as a brown gum

LC-MS (ESI): m/z=284.30 [M+H]⁺

### Preparation of tert-butyl 4-((4-(3-(2,6-bis(benzyloxy)pyridin-3-yl)-7-fluoro-1-methyl-1H-indazol-6-yl)piperazin-1-yl)methyl)piperidine-1-carboxylate

A stirred solution of tert-butyl 4-(piperazin-1-ylmethyl)piperidine-1-carboxylate (1.9 g, 6.70 mmol) and 3-(2,6-bis(benzyloxy)pyridin-3-yl)-6-bromo-7-fluoro-1-methyl-1H-indazole (3.68 g, 7.37 mmol) in 1,4-dioxane (6.0 mL) was degassed using nitrogen for 15 min. To this mixture was added BrettPhos Pd G3 (0.60 g, 0.67 mmol) and NaOtBu (1.93 g, 20.1 mmol). The mixture was stirred at 100° C. for 10 h. The reaction mixture was filtered through celite, and the filtrate was concentrated under reduced pressure to get a crude product which was purified by flash column chromatography (20-30% ethyl acetate in petroleum ether) to afford tert-butyl 4-((4-(3-(2,6-bis(benzyloxy)pyridin-3-yl)-7-fluoro-1-methyl-1H-indazol-6-yl)piperazin-1-yl)methyl)piperidine-1-carboxylate (0.65 g) as a brown gum

LC-MS (ESI): m/z=721.68 [M+H]⁺

### Preparation of tert-butyl 4-((4-(3-(2,6-dioxopiperidin-3-yl)-7-fluoro-1-methyl-1H-indazol-6-yl)piperazin-1-yl)methyl)piperidine-1-carboxylate

To a stirred solution of tert-butyl 4-((4-(3-(2,6-bis(benzyloxy)pyridin-3-yl)-7-fluoro-1-methyl-1H-indazol-6-yl)piperazin-1-yl)methyl)piperidine-1-carboxylate (0.65 g, 0.90 mmol) in THF (6.5 mL) was added 20% Pd(OH)₂ (100% w/w) (0.65 g). The mixture was kept under hydrogen pressure (90 psi) at room temperature and stirred for 16 h. The reaction mixture was filtered through celite, and the filtrate was concentrated under reduced pressure to get tert-butyl 4-((4-(3-(2,6-dioxopiperidin-3-yl)-7-fluoro-1-methyl-1H-indazol-6-yl)piperazin-1-yl)methyl)piperidine-1-carboxylate (0.3 g) as an off white solid

LC-MS (ESI): m/z=543.46 [M+H]⁺

### Preparation of 3-(7-fluoro-1-methyl-6-(4-(piperidin-4-ylmethyl)piperazin-1-yl)-1H-indazol-3-yl)piperidine-2,6-dione

To a stirred solution of tert-butyl 4-((4-(3-(2,6-dioxopiperidin-3-yl)-7-fluoro-1-methyl-1H-indazol-6-yl)piperazin-1-yl)methyl)piperidine-1-carboxylate (0.3 g, 0.55 mmol) in DCM (3.0 ml) was added TFA (3.0 ml) at 0° C. The reaction mixture was stirred at room temperature for 3 h.

The reaction mixture was concentrated under reduced pressure to afford 3-(7-fluoro-1-methyl-6-(4-(piperidin-4-ylmethyl)piperazin-1-yl)-1H-indazol-3-yl)piperidine-2,6-dione (0.3 g) as a brown solid.

LC-MS (ESI): m/z=443.40 [M+H]⁺

### Preparation of 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-7-fluoro-1-methyl-1H-indazol-3-yl)piperidine-2,6-dione

To a stirred solution of 3-(7-fluoro-1-methyl-6-(4-(piperidin-4-ylmethyl)piperazin-1-yl)-1H-indazol-3-yl)piperidine-2,6-dione (0.3 g, 0.33 mmol) in dry DMSO (1.5 mL) was added N,N-diisopropylethylamine (1.5 mL) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.15 g, 0.33 mmol) at room temperature. The mixture was heated at 110° C. and stirred for 4 h. The reaction mixture was cooled to room temperature, concentrated under reduced pressure, and purified by prep-HPLC to afford 3-(6-(4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)piperazin-1-yl)-7-fluoro-1-methyl-1H-indazol-3-yl)piperidine-2,6-dione (0.036 g) as an off-white solid.

LC-MS (ESI): m/z=874.37, [M+H]⁺

¹H NMR (400 MHz, DMSO-d₆): δ 10.89 (s, 1H), 8.77 (s, 1H), 8.18 (d, J=1.60 Hz, 1H), 8.03 (s, 1H), 7.74 (dd, J=1.60, 9.00 Hz, 1H), 7.43 (dd, J=8.80, 16.20 Hz, 2H), 6.91 (t, J=8.00 Hz, 1H), 6.16 (s, 1H), 4.31-4.30 (m, 5H), 4.06 (s, 3H), 3.56 (s, 3H), 3.23-3.21 (m, 1H), 3.09 (s, 4H), 2.79-2.77 (m, 2H), 2.51-2.46 (m, 5H), 2.33-2.28 (m, 2H), 2.15-2.10 (m, 3H), 1.75-1.67 (m, 3H), 1.34-1.23 (m, 1H), 1.03-1.02 (m, 2H), 0.73-0.63 (m, 1H), 0.52-0.43 (m, 2H), 0.40-0.31 (m, 1H).

### Example 180: 3-(6-(1-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione (Compound 525d)

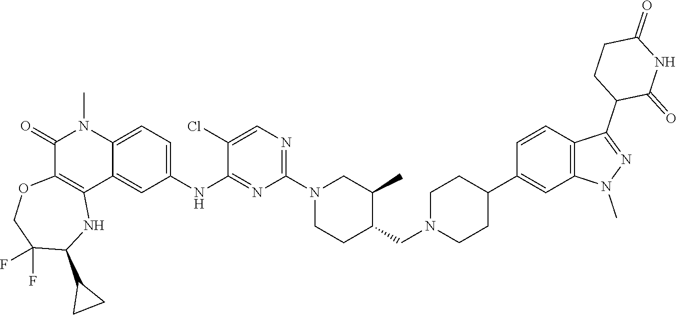 [SMILES: C[C@@H]1CN(c2ncc(Cl)c(Nc3ccc4c(c3)c3c(c(=O)n4C)OCC(F)(F)[C@H](C4CC4)N3)n2)CC[C@H]1CN1CCC(c2ccc3c(C4CCC(=O)NC4=O)nn(C)c3c2)CC1]

### Preparation of tert-butyl (3S, 4R)-4-(4-(3-(2,6-bis(benzyloxy)pyridin-3-yl)-1-methyl-1H-indazol-6-yl)piperidine-1-carbonyl)-3-methylpiperidine-1-carboxylate

To a stirred solution of (3S, 4R)-1-(tert-butoxycarbonyl)-3-methylpiperidine-4-carboxylic acid (0.09 g, 0.35 mmol), and 3-(2,6-bis(benzyloxy)pyridin-3-yl)-1-methyl-6-(1,2,3,6-tetrahydropyridin-4-yl)-1H-indazole (0.30 g, 0.60 mmol) in DMF (6.0 mL) was added DIPEA (0.3 mL, 1.79 mmol). The mixture was stirred for 10 min followed by the addition of HATU (0.34 g, 0.90 mmol) at 0° C. The mixture was stirred at room temperature for 3 h. The reaction was quenched with cold water, filtered, and dried to afford tert-butyl (3S, 4R)-4-(4-(3-(2,6-bis(benzyloxy)pyridin-3-yl)-1-methyl-1H-indazol-6-yl)piperidine-1-carbonyl)-3-methylpiperidine-1-carboxylate (0.42 g) as an off white solid.

LC-MS (ESI): m/z: 728.35 [M+H]⁺

### Preparation of tert-butyl (3S, 4R)-4-((4-(3-(2,6-bis(benzyloxy)pyridin-3-yl)-1-methyl-1H-indazol-6-yl)piperidin-1-yl)methyl)-3-methylpiperidine-1-carboxylate

To a stirred solution of tert-butyl (3S, 4R)-4-(4-(3-(2,6-bis(benzyloxy)pyridin-3-yl)-1-methyl-1H-indazol-6-yl)piperidine-1-carbonyl)-3-methylpiperidine-1-carboxylate (0.42 g, 0.57 mmol) in THF (8.4 mL) was added 2.0 M LiAlH₄ in THF (0.63 mL). The mixture was stirred at room temperature for 2 h. The reaction was diluted with ethyl acetate (40 mL) and washed with water (20 mL). The organic layer was separated and dried over anhydrous sodium sulfate and concentrated under reduced pressure to afford tert-butyl (3S, 4R)-4-((4-(3-(2,6-bis(benzyloxy)pyridin-3-yl)-1-methyl-1H-indazol-6-yl)piperidin-1-yl)methyl)-3-methylpiperidine-1-carboxylate (0.35 g) as a brown sticky compound.

LC-MS (ESI): m/z: 714.78 [M+H]⁺

### Preparation of tert-butyl (3S, 4R)-4-((4-(3-(2,6-dioxopiperidin-3-yl)-1-methyl-1H-indazol-6-yl)piperidin-1-yl)methyl)-3-methylpiperidine-1-carboxylate

To a stirred solution of tert-butyl (3S, 4R)-4-((4-(3-(2,6-bis(benzyloxy)pyridin-3-yl)-1-methyl-1H-indazol-6-yl)piperidin-1-yl)methyl)-3-methylpiperidine-1-carboxylate (0.35 g, 0.49 mmol) in THF (21 mL) was added 20% Pd (OH)₂ on carbon, moisture 50% wet (0.35 g, 100% w/w). The mixture was stirred under hydrogen atmosphere (80 psi) at room temperature for 16 h. The reaction mixture was diluted with ethyl acetate (50 mL), filtered through a celite bed, and washed with THF:DCM (200 mL). The filtrate was concentrated and triturated with diethyl ether (3×15 mL) to afford tert-butyl (3S, 4R)-4-((4-(3-(2,6-dioxopiperidin-3-yl)-1-methyl-1H-indazol-6-yl)piperidin-1-yl)methyl)-3-methylpiperidine-1-carboxylate (0.25 g) as a brown sticky compound.

LC-MS (ESI): m/z=538.66 [M+H]⁺

### Preparation of 3-(1-methyl-6-(1-(((3S, 4R)-3-methylpiperidin-4-yl)methyl)piperidin-4-yl)-1H-indazol-3-yl)piperidine-2,6-dione

To a stirred solution of tert-butyl (3S, 4R)-4-((4-(3-(2,6-dioxopiperidin-3-yl)-1-methyl-1H-indazol-6-yl)piperidin-1-yl)methyl)-3-methylpiperidine-1-carboxylate (0.25 g, 0.46 mmol) in DCM (2.50 mL) was added TFA (1.25 mL). The mixture was stirred at room temperature for 2 h. The reaction mixture was concentrated under reduced pressure to obtain a crude product, which was triturated with diethyl ether (15 mL) to afford 3-(1-methyl-6-(1-(((3S, 4R)-3-methylpiperidin-4-yl)methyl)piperidin-4-yl)-1H-indazol-3-yl)piperidine-2,6-dione (0.18 g) as a brown solid.

LC-MS (ESI): m/z: 438.35 [M+H]⁺

### Preparation of 3-(7-(1-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione

### [3-(6-(1-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione]

To a stirred solution of 3-(1-methyl-6-(1-(((3S, 4R)-3-methylpiperidin-4-yl)methyl)piperidin-4-yl)-1H-indazol-3-yl)piperidine-2,6-dione (0.18 g, 0.41 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.04 g, 0.08 mmol) in DMSO (3.6 mL) was added N,N-diisopropylethylamine (1.8 mL). The mixture was stirred at 100° C. for 6 h. The reaction mixture was poured into cold water and stirred for 15 minutes. The precipitated solid was filtered and dried to obtain crude compound which was purified by prep-HPLC to afford 3-(7-(1-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione [3-(6-(1-(((3S, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperidin-4-yl)-1-methyl-1H-indazol-3-yl)piperidine-2,6-dione](0.017 g) as an off-white solid.

LC-MS (ESI): m/z: 869.39 [M+H]⁺

¹H NMR (400 MHz, DMSO-d6): δ 10.89 (s, 1H), 8.80 (s, 1H), 8.18 (s, 1H), 8.03 (s, 1H), 7.73 (d, J=9.20 Hz, 1H), 7.60 (d, J=8.00 Hz, 1H), 7.46 (s, 1H), 7.43 (d, J=5.60 Hz, 1H), 7.02 (d, J=8.40 Hz, 1H), 6.14 (s, 1H), 4.45-4.31 (m, 4H), 3.96 (s, 3H), 3.57 (s, 3H), 3.33-3.19 (m, 2H), 2.96-3.02 (m, 1H), 2.80-2.82 (m, 2H), 2.60-2.51 (m, 3H), 2.43-2.38 (m, 2H), 2.18-2.06 (m, 3H), 1.91-1.76 (m, 6H), 1.39-1.30 (m, 2H), 1.23-1.20 (m, 2H), 0.77-0.68 (m, 1H), 0.89-087 (m, 3H), 0.74-0.71 (m, 1H), 0.53-0.52 (m, 2H), 0.39-0.35 (m, 1H).

### Example 201: 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-hydroxypiperidin-4-yl)-1-methyl-1H-indazol-3-yl)piperidine-2,6-dione (Compound 526a)

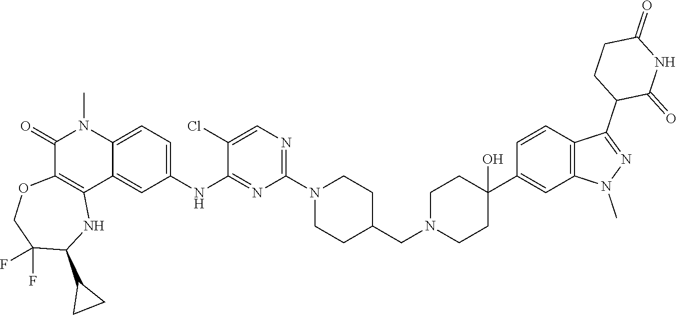 [SMILES: Cn1nc(C2CCC(=O)NC2=O)c2ccc(C3(O)CCN(CC4CCN(c5ncc(Cl)c(Nc6ccc7c(c6)c6c(c(=O)n7C)OCC(F)(F)[C@H](C7CC7)N6)n5)CC4)CC3)cc21]

### Preparation of tert-butyl 4-(3-(2,6-bis(benzyloxy)pyridin-3-yl)-1-methyl-1H-indazol-6-yl)-3,6-dihydropyridine-1(2H)-carboxylate

To a stirred solution of 3-(2,6-bis(benzyloxy)pyridin-3-yl)-6-bromo-1-methyl-1H-indazole (5.0 g, 9.992 mmol) and tert-butyl 4-(4,4,5,5-tetramethyl-1,3,2-dioxaborolan-2-yl)-3,6-dihydropyridine-1(2H)-carboxylate (3.70 g, 11.991 mmol) in 1,4-dioxane (200 mL) and water (100 mL) was added cesium carbonate (9.76 g, 29.976 mmol). The reaction mixture was degassed for 10 min followed by the addition of Pd₂(dba)₃ (0.45 g, 0.500 mmol) and xantphos (0.57 g, 0.999 mmol). The mixture was stirred at 100° C. for 6 h. The reaction mixture was concentrated under reduced pressure and the crude was diluted with water (100 mL) and extracted with DCM (3×150 mL). The combined organic layers were washed with brine (100 mL), dried over anhydrous Na₂SO₄, filtered, and dried under vacuum to get a crude product which was purified by column chromatography (30% ethyl acetate in petroleum ether) to afford tert-butyl 4-(3-(2,6-bis(benzyloxy)pyridin-3-yl)-1-methyl-1H-indazol-6-yl)-3,6-dihydropyridine-1(2H)-carboxylate (5.0 g) as a yellow solid.

LC-MS (ESI): m/z=603.43 [M+H]⁺

### Preparation of tert-butyl 4-(3-(2,6-bis(benzyloxy)pyridin-3-yl)-1-methyl-1H-indazol-6-yl)-4-hydroxypiperidine-1-carboxylate

To a stirred solution of tert-butyl 4-(3-(2,6-bis(benzyloxy)pyridin-3-yl)-1-methyl-1H-indazol-6-yl)-3,6-dihydropyridine-1(2H)-carboxylate (3.0 g, 4.97 mmol) in isopropyl alcohol (24.0 mL) and dichloromethane (6.0 mL) was added tris(2,2,6,6-tetramethyl-3,5-heptanedionato)manganese(III) (0.150 g, 0.249 mmol) and phenylsilane (1.857 mL, 14.93 mmol) at 0° C. under oxygen atmosphere. The mixture was stirred at 0° C. for 4 h. The reaction mixture was quenched with water (50 mL) and extracted with DCM (3×50 mL). The combined organic layers were washed with brine (50 mL), dried over anhydrous Na₂SO₄, filtered, and dried under vacuum to get a crude product which was purified by flash column chromatography (45% ethyl acetate in petroleum ether) to afford tert-butyl 4-(3-(2,6-bis(benzyloxy)pyridin-3-yl)-1-methyl-1H-indazol-6-yl)-4-hydroxypiperidine-1-carboxylate (2.0 g).

LC-MS (ESI): m/z=621.52 [M+H]⁺

### Preparation of 4-(3-(2,6-bis(benzyloxy)pyridin-3-yl)-1-methyl-1H-indazol-6-yl)piperidin-4-ol

To a stirred solution of tert-butyl 4-(3-(2,6-bis(benzyloxy)pyridin-3-yl)-1-methyl-1H-indazol-6-yl)-4-hydroxypiperidine-1-carboxylate (2.0 g, 3.22 mmol) in DCM (20 mL) was added TFA (4.0 mL). The mixture was stirred at room temperature for 2 h. The reaction mixture was concentrated under reduced pressure to obtain a crude product which was triturated with n-pentane (10.0 ml) and dried to afford 4-(3-(2,6-bis(benzyloxy)pyridin-3-yl)-1-methyl-1H-indazol-6-yl)piperidin-4-ol (1.5 g) as a yellow solid.

LC-MS (ESI): m/z=521.34 [M+H]⁺

### Preparation of tert-butyl 4-((4-(3-(2,6-bis(benzyloxy)pyridin-3-yl)-1-methyl-1H-indazol-6-yl)-4-hydroxypiperidin-1-yl)methyl)piperidine-1-carboxylate

To a stirred solution of 4-(3-(2,6-bis(benzyloxy)pyridin-3-yl)-1-methyl-1H-indazol-6-yl)piperidin-4-ol (1.5 g, 2.88 mmol) and tert-butyl 4-formylpiperidine-1-carboxylate (0.73 g, 3.45 mmol) in THF (15.00 mL) was added acetic acid (0.16 mL, 2.88 mmol) sodium acetate (anhydrous) (0.71 g, 8.64 mmol), and sodium triacetoxyborohydride, (1.83 g, 8.64 mmol) at 0° C.

The mixture was stirred at room temperature for 12 h. The reaction mixture was quenched with water (30.0 mL) and extracted with DCM (50.0 mL). The separated organic layer was washed with brine (20.0 mL), dried over anhydrous Na₂SO₄, filtered, and dried under vacuum to get a crude product which was purified by flash column chromatography (80% ethyl acetate in petroleum ether) to afford tert-butyl 4-((4-(3-(2,6-bis(benzyloxy)pyridin-3-yl)-1-methyl-1H-indazol-6-yl)-4-hydroxypiperidin-1-yl)methyl)piperidine-1-carboxylate (1.4 g) as a yellow solid.

LC-MS (ESI): m/z=628.43 [M-Bn+H]⁺

### Preparation of tert-butyl 4-((4-(3-(2,6-dioxopiperidin-3-yl)-1-methyl-1H-indazol-6-yl)-4-hydroxypiperidin-1-yl)methyl)piperidine-1-carboxylate

To a stirred solution of tert-butyl 4-((4-(3-(2,6-bis(benzyloxy)pyridin-3-yl)-1-methyl-1H-indazol-6-yl)-4-hydroxypiperidin-1-yl)methyl)piperidine-1-carboxylate (1.4 g, 1.95 mmol) in THF (28.0 mL) was added 20% palladium hydroxide on carbon (1.4 g, 100% w/w). The mixture was stirred under H₂ (80 psi) pressure at room temperature for 6 h. The reaction mixture was filtered using celite and washed with ethyl acetate (10 mL). The filtrate was concentrated under vacuum to get a crude product which was purified by flash column chromatography (100% ethyl acetate) to afford tert-butyl 4-((4-(3-(2,6-dioxopiperidin-3-yl)-1-methyl-1H-indazol-6-yl)-4-hydroxypiperidin-1-yl)methyl)piperidine-1-carboxylate (0.93 g) as an off white solid.

LC-MS (ESI): m/z=540.50 [M+H]⁺

### Preparation of 3-(6-(4-hydroxy-1-(piperidin-4-ylmethyl)piperidin-4-yl)-1-methyl-1H-indazol-3-yl)piperidine-2,6-dione

To a stirred solution of tert-butyl 4-((4-(3-(2,6-dioxopiperidin-3-yl)-1-methyl-1H-indazol-6-yl)-4-hydroxypiperidin-1-yl)methyl)piperidine-1-carboxylate (0.93 g, 1.72 mmol) in DCM (10 mL. TFA (5 mL) was added at 0° C. The mixture was stirred at room temperature for 2 h. The reaction mixture was concentrated under reduced pressure to obtain crude product which was triturated with n-pentane (10.0 mL) and dried to afford 3-(6-(4-hydroxy-1-(piperidin-4-ylmethyl)piperidin-4-yl)-1-methyl-1H-indazol-3-yl)piperidine-2,6-dione (0.7 g) as an off white solid.

LC-MS (ESI): m/z=440.50 [M+H]⁺

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-hydroxypiperidin-4-yl)-1-methyl-1H-indazol-3-yl)piperidine-2,6-dione

To a stirred solution of 3-(6-(4-hydroxy-1-(piperidin-4-ylmethyl)piperidin-4-yl)-1-methyl-1H-indazol-3-yl)piperidine-2,6-dione (0.2 g, 0.45 mmol) and (S)-2-cyclopropyl-10-((2,5-dichloropyrimidin-4-yl)amino)-3,3-difluoro-7-methyl-1,2,3,4-tetrahydro[1,4]oxazepino[2,3-c]quinolin-6(7H)-one (0.08 g, 0.18 mmol)) in DMSO (4.0 mL) was added DIPEA (0.24 mL, 1.82 mmol). The mixture was heated at 100° C. and stirred for 3 h. The reaction mixture was quenched with ice-cold water, and the solid was filtered to get crude product which was purified by prep-HPLC to afford 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)-4-hydroxypiperidin-4-yl)-1-methyl-1H-indazol-3-yl)piperidine-2,6-dione (0.072 g) as an off-white solid.

LC-MS (ESI): m/z=871.37 [M+H]⁺

¹H NMR 400 MHz, DMSO-d6: δ 10.88 (s, 1H), 8.77 (s, 1H), 8.18 (bs, 3H), 8.02 (s, 1H), 7.44 (dd, J=7.2, 2.0 Hz, 1H), 7.70-7.60 (m, 2H), 7.45 (d, J=9.2 Hz, 1H), 7.26 (d, J=9.2 Hz, 1H), 6.14 (s, 1H), 4.9 (bs, 1H), 4.46-4.30 (m, 4H), 3.96 (s, 3H), 3.57 (s, 3H), 2.94 (bs, 2H), 2.81-2.75 (m, 2H), 2.67-2.60 (m, 3H), 2.50-2.30 (m, 2H), 2.30-2.20 (m, 3H), 2.20-1.98 (m, 2H), 1.78-1.72 (m, 3H), 1.60-1.50 (m, 2H), 1.40-1.39 (m, 1H), 1.12-0.98 (m, 2H), 0.83-0.76 (m, 1H), 0.61-0.59 (m, 2H), 0.40-0.36 (m, 1H).

### Example 231. 3-(6-(4-((9-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3,9-diazaspiro[5.5]undecan-3-yl)methyl)piperidin-1-yl)-7-fluoro-1-methyl-1H-indazol-3-yl)piperidine-2,6-dione (Compound 565a)

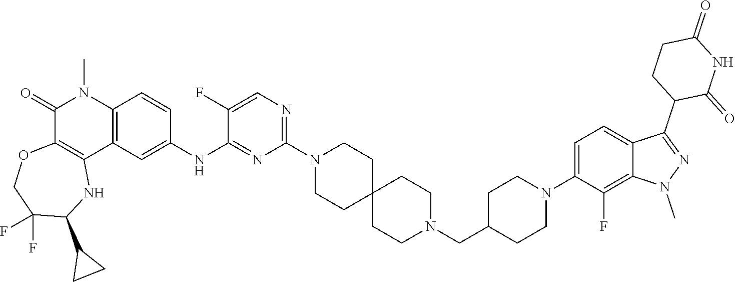 [SMILES: Cn1nc(C2CCC(=O)NC2=O)c2ccc(N3CCC(CN4CCC5(CC4)CCN(c4ncc(F)c(Nc6ccc7c(c6)c6c(c(=O)n7C)OCC(F)(F)[C@H](C7CC7)N6)n4)CC5)CC3)c(F)c21]

### Preparation of 3-(6-(4-((9-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3,9-diazaspiro[5.5]undecan-3-yl)methyl)piperidin-1-yl)-7-fluoro-1-methyl-1H-indazol-3-yl)piperidine-2,6-dione

To a solution of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (165 mg, 1 equiv., 365 mol) in DMSO (1.25 mL) was added 3-(6-(4-((3,9-diazaspiro[5.5]undecan-3-yl)methyl)piperidin-1-yl)-7-fluoro-1-methyl-1H-indazol-3-yl)piperidine-2,6-dione, 3HCl (272 mg, 1.2 equiv., 438 mol) and DIEA (236 mg, 318 μL, 5.0 equiv., 1.83 mmol). The reaction mixture was stirred at 80° C. for 18 h. The reaction mixture was filtered and purified by reverse phase HPLC to give a brown solid which was taken up in acetonitrile:water, frozen, and lyophilized to give 3-(6-(4-((9-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3,9-diazaspiro[5.5]undecan-3-yl)methyl)piperidin-1-yl)-7-fluoro-1-methyl-1H-indazol-3-yl)piperidine-2,6-dione (89 mg) as a gray solid.

¹H NMR (400 MHz, DMSO-d6) δ (ppm)=10.88 (s, 1H), 9.26 (s, 1H), 8.24 (d, J=1.9 Hz, 1H), 7.99 (d, J=3.6 Hz, 1H), 7.76 (dd, J=1.9, 9.0 Hz, 1H), 7.44 (d, J=9.1 Hz, 1H), 7.38 (d, J=8.8 Hz, 1H), 6.96-6.87 (m, 1H), 6.00 (br s, 1H), 4.52-4.34 (m, 2H), 4.31 (dd, J=5.1, 9.9 Hz, 1H), 4.06 (s, 3H), 3.63-3.57 (m, 3H), 3.58-3.54 (m, 4H), 3.41-3.34 (m, 3H), 3.26-3.18 (m, 1H), 2.77 (br t, J=11.0 Hz, 2H), 2.72-2.55 (m, 2H), 2.41-2.26 (m, 5H), 2.23-2.10 (m, 3H), 1.79 (br d, J=11.8 Hz, 2H), 1.63 (br dd, J=3.8, 6.5 Hz, 1H), 1.52-1.37 (m, 7H), 1.36-1.20 (m, 3H), 0.78-0.65 (m, 1H), 0.58-0.45 (m, 2H), 0.43-0.31 (m, 1H).

LC-MS (ESI): m/z=926.6 [M+1]⁺

### Example 232. 3-(6-(4-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)methyl)piperazin-1-yl)-1-methyl-1H-indazol-3-yl)piperidine-2,6-dione (Compound 516a)

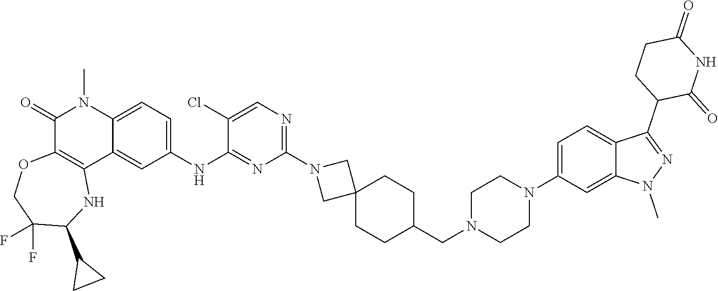 [SMILES: Cn1nc(C2CCC(=O)NC2=O)c2ccc(N3CCN(CC4CCC5(CC4)CN(c4ncc(Cl)c(Nc6ccc7c(c6)c6c(c(=O)n7C)OCC(F)(F)[C@H](C7CC7)N6)n4)C5)CC3)cc21]

### Preparation of tert-butyl 7-((4-(3-(2,6-dioxopiperidin-3-yl)-1-methyl-1H-indazol-6-yl)piperazin-1-yl)methyl)-2-azaspiro[3.5]nonane-2-carboxylate

A mixture of tert-butyl 7-formyl-2-azaspiro[3.5]nonane-2-carboxylate (110 mg, 433 mol), 3-(1-methyl-6-(piperazin-1-yl)-1H-indazol-3-yl)piperidine-2,6-dione hydrochloride (105 mg, 289 mol), and potassium acetate (85.0 mg, 866 mol) was suspended in THF (1.50 mL) and DMF (0.225 mL). The mixture was stirred at room temperature for 10 min. The white mixture was then treated all at once with sodium triacetoxyborohydride (91.7 mg, 433 mol). The resulting mixture was allowed to stir at room temperature for 24 h. The reaction mixture was treated with saturated aqueous ammonium chloride solution (1 mL), then extracted with ethyl acetate (3×). The combined organics were dried over sodium sulfate, filtered, concentrated under reduced pressure, and purified by column chromatography to afford tert-butyl 7-((4-(3-(2,6-dioxopiperidin-3-yl)-1-methyl-1H-indazol-6-yl)piperazin-1-yl)methyl)-2-azaspiro[3.5]nonane-2-carboxylate (113.0 mg). LC-MS (ESI): m/z=565.3 [M+H]⁺.

### Preparation of 3-(6-(4-((2-azaspiro[3.5]nonan-7-yl)methyl)piperazin-1-yl)-1-methyl-1H-indazol-3-yl)piperidine-2,6-dione bis(trifluoroacetate)

A solution of tert-butyl 7-((4-(3-(2,6-dioxopiperidin-3-yl)-1-methyl-1H-indazol-6-yl)piperazin-1-yl)methyl)-2-azaspiro[3.5]nonane-2-carboxylate (111.9 mg, 198.1 mol) and DCM (1.00 mL) was treated with a solution of TFA (1.00 mL) and DCM (1.00 mL). The mixture was stirred at room temperature for 2 h. The volatiles were removed in vacuo to afford 3-(6-(4-((2-azaspiro[3.5]nonan-7-yl)methyl)piperazin-1-yl)-1-methyl-1H-indazol-3-yl)piperidine-2,6-dione bis(trifluoroacetate) (137 mg). LC-MS (ESI): m/z=465.4 [M+H]⁺.

### Preparation of 3-(6-(4-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)methyl)piperazin-1-yl)-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (48.7 mg, 104 mol), 3-(6-(4-((2-azaspiro[3.5]nonan-7-yl)methyl)piperazin-1-yl)-1-methyl-1H-indazol-3-yl)piperidine-2,6-dione bis(trifluoroacetate) (50.0 mg, 72.2 mol), and CsF (47.0 mg, 309 mol) in DMSO (0.800 mL) was treated with DIEA (75.4 μL, 433 mol). The reaction mixture was warmed to 80° C. for 15 h, and then cooled to room temperature. The reaction mixture was filtered and purified by prep-HPLC to afford 3-(6-(4-((2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-2-azaspiro[3.5]nonan-7-yl)methyl)piperazin-1-yl)-1-methyl-1H-indazol-3-yl)piperidine-2,6-dione (46.7 mg). ¹H NMR (400 MHz, DMSO-d6): δ 10.77 (s, 1H), 8.66 (s, 1H), 8.18 (s, 1H), 7.95 (s, 1H), 7.76 (d, J=9.0 Hz, 1H), 7.43 (d, J=8.9 Hz, 1H), 7.37 (d, J=9.2 Hz, 1H), 6.84 (d, J=9.0 Hz, 1H), 6.76 (s, 1H), 6.04 (s, 1H), 4.46-4.24 (m, 2H), 4.19 (dd, J=9.2, 5.0 Hz, 1H), 3.82 (s, 3H), 3.57 (s, 2H), 3.53 (s, 2H), 3.50 (s, 3H), 3.19-2.93 (m, 6H), 2.60-2.47 (m, 2H), 2.29-2.16 (m, 1H), 2.13-2.00 (m, 4H), 1.85-1.71 (m, 2H), 1.71-1.58 (m, 2H), 1.51-1.30 (m, 4H), 1.30-1.17 (m, 2H), 0.97-0.76 (m, 2H), 0.72-0.59 (m, 1H), 0.46 (t, J=6.2 Hz, 2H), 0.34-0.25 (m, 1H). LC-MS (ESI): m/z=896.5 [M+H]⁺.

### Example 233. 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperidin-4-yl)-1-methyl-1H-indazol-3-yl)piperidine-2,6-dione (Compound 495a)

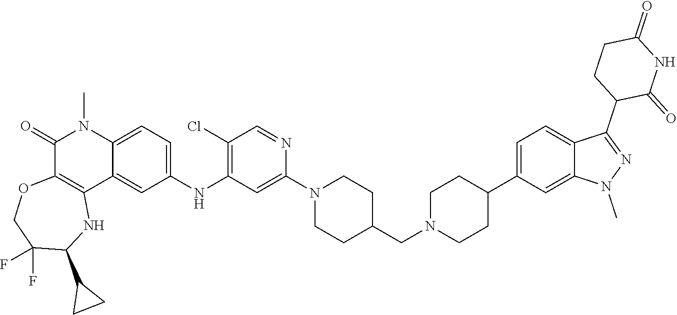 [SMILES: Cn1nc(C2CCC(=O)NC2=O)c2ccc(C3CCN(CC4CCN(c5cc(Nc6ccc7c(c6)c6c(c(=O)n7C)OCC(F)(F)[C@H](C7CC7)N6)c(Cl)cn5)CC4)CC3)cc21]

### Preparation of 2-(4-(1,3-dioxolan-2-yl)piperidin-1-yl)-5-chloro-4-iodopyridine

A solution of 5-chloro-2-fluoro-4-iodopyridine (2.177 g, 8.457 mmol) and 4-(1,3-dioxolan-2-yl)piperidine (1.2046 g, 7.6624 mmol) in DMSO (5.00 mL) was treated with DIEA (4.00 mL, 22.987 mmol). The reaction mixture was stirred at room temperature for 16 h. The reaction mixture was diluted with water and then extracted with DCM (3×). The combined organics were dried over Na₂SO₄, filtered, and concentrated under reduced pressure to afford a pale yellow solid, which was purified by column chromatography (0-60% ethyl acetate/heptane) to afford 2-(4-(1,3-dioxolan-2-yl)piperidin-1-yl)-5-chloro-4-iodopyridine (2.036 g) as a white solid. LC-MS (ESI): m/z=394.9 [M+H]⁺.

### Preparation of (S)-10-((2-(4-(1,3-dioxolan-2-yl)piperidin-1-yl)-5-chloropyridin-4-yl)amino)-2-cyclopropyl-3,3-difluoro-7-methyl-1,2,3,4-tetrahydro-[1,4]oxazepino[2,3-c]quinolin-6(7H)-one

A mixture of (S)-10-amino-2-cyclopropyl-3,3-difluoro-7-methyl-1,2,3,4-tetrahydro-[1,4]oxazepino[2,3-c]quinolin-6(7H)-one (185 mg, 576 mol), 2-(4-(1,3-dioxolan-2-yl)piperidin-1-yl)-5-chloro-4-iodopyridine (210 mg, 532 mol), and BrettPhos-Pd-G3 (99.7 mg, 110 mol) in 1,4-dioxane (4.00 mL) was sparged with N₂ for 15 min. The mixture was then treated with NaOtBu, 2.0 M in THF (798 μL, 1.60 mmol) while sparging with N₂. The reaction mixture was sparged for another two minutes and then warmed to 100° C. for 1 h. The reaction mixture was allowed to cool to room temperature, treated with formic acid (73.5 mg, 60.2 μL, 3.00 equiv., 1.60 mmol), diluted with DMSO (3 mL), filtered through a pad of celite, and rinsed thoroughly with MeOH. The combined organics were concentrated to a viscous solution under reduced pressure.

The material was then purified by prep-HPLC to afford (S)-10-((2-(4-(1,3-dioxolan-2-yl)piperidin-1-yl)-5-chloropyridin-4-yl)amino)-2-cyclopropyl-3,3-difluoro-7-methyl-1,2,3,4-tetrahydro-[1,4]oxazepino[2,3-c]quinolin-6(7H)-one (61.0 mg). LC-MS (ESI): m/z=588.2 [M+H]⁺.

### Preparation of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde

A solution of (S)-10-((2-(4-(1,3-dioxolan-2-yl)piperidin-1-yl)-5-chloropyridin-4-yl)amino)-2-cyclopropyl-3,3-difluoro-7-methyl-1,2,3,4-tetrahydro-[1,4]oxazepino[2,3-c]quinolin-6(7H)-one (59.0 mg, 1.00 equiv., 100 mol) and formic acid (2.44 g, 2.00 mL, 528 equiv., 53.0 mmol) was warmed to 80° C. for 2 h and then cooled to room temperature. The volatiles were removed under reduced pressure to afford (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde (66.3 mg). LC-MS (ESI): m/z=544.2 [M+H]⁺.

### Preparation of 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperidin-4-yl)-1-methyl-1H-indazol-3-yl)piperidine-2,6-dione

A mixture of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde (54.6 mg, 100 mol), 3-(1-methyl-6-(piperidin-4-yl)-1H-indazol-3-yl)piperidine-2,6-dione trifluoroacetate (57.3 mg, 130 mol), and potassium acetate (37.6 mg, 383 mol) in THF (1.00 mL) and DMF (0.150 mL) was stirred at room temperature for 30 min. The mixture was then treated with sodium triacetoxyborohydride (47.0 mg, 222 mol). The reaction mixture was allowed to stir at room temperature for 45 min. The reaction mixture was quenched by the addition of aqueous ammonium chloride solution (4 mL) and then extracted with DCM (5×). The aqueous layer was neutralized with NaHCO₃(aq, sat) and again extracted with DCM (3×). The combined organics were treated with formic acid (10 microliters), dried over sodium sulfate, filtered, and concentrated under reduced pressure in the presence of 2 mL DMSO. The crude material was filtered and then purified by prep-HPLC to afford 3-(6-(1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)piperidin-4-yl)-1-methyl-1H-indazol-3-yl)piperidine-2,6-dione (44.0 mg). LC-MS (ESI): m/z=854.5 [M+H]⁺.

### Example 234. 3-(6-((S)-4-(((S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)pyrrolidin-3-yl)methyl)-3-methylpiperazin-1-yl)-1-methyl-1H-indazol-3-yl)piperidine-2,6-dione (Compound 528a)

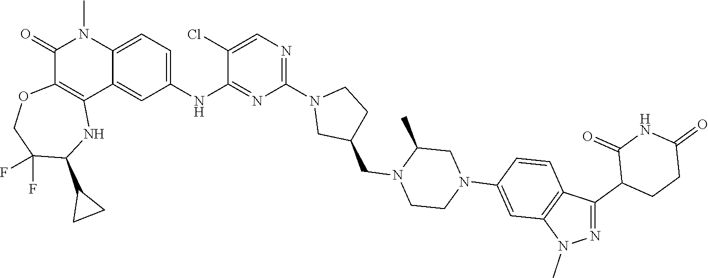 [SMILES: C[C@H]1CN(c2ccc3c(C4CCC(=O)NC4=O)nn(C)c3c2)CCN1C[C@@H]1CCN(c2ncc(Cl)c(Nc3ccc4c(c3)c3c(c(=O)n4C)OCC(F)(F)[C@H](C4CC4)N3)n2)C1]

### Preparation of tert-butyl (S)-4-(3-(2,6-bis(benzyloxy)pyridin-3-yl)-1-methyl-1H-indazol-6-yl)-2-methylpiperazine-1-carboxylate

A mixture of 3-(2,6-bis(benzyloxy)pyridin-3-yl)-6-bromo-1-methyl-1H-indazole (15.0 g, 29.9 mmol, 1.00 equiv.), tert-butyl (S)-2-methylpiperazine-1-carboxylate (12.0 g, 59.9 mmol, 2.00 equiv.), Pd₂(dba)₃ (2.75 g, 3.00 mmol, 0.10 equiv.), RuPhos (1.40 g, 3.00 mmol, 0.10 equiv.), and t-BuONa (8.64 g, 89.9 mmol, 3.00 equiv.) in dioxane (150 mL) was degassed and purged with N₂ three times. The mixture was stirred at 100° C. for 1 h under N₂ atmosphere. The reaction mixture was quenched by the addition of H₂O (40.0 mL) at 25° C., diluted with ethyl acetate (100 mL), and extracted with ethyl acetate (100 mL×2). The combined organic layers were washed with brine (100 mL×3), dried over anhydrous Na₂SO₄, filtered, and concentrated under reduced pressure to give a residue. The residue was purified by column chromatography (petroleum ether:ethyl acetate=5:1 to 1:1) to give tert-butyl (S)-4-(3-(2,6-bis(benzyloxy)pyridin-3-yl)-1-methyl-1H-indazol-6-yl)-2-methylpiperazine-1-carboxylate (18.5 g, 25.3 mmol) as a yellow oil.

LC-MS (ESI): m/z=621.2 [M+H]⁺

¹H NMR: (400 MHz, DMSO-d₆) δ 7.91-7.85 (m, 1H), 7.53-7.52 (m, 1H), 7.52 (d, J=9.0 Hz, 1H), 7.48-7.43 (m, 2H), 7.42-7.34 (m, 5H), 7.33-7.25 (m, 4H), 6.84 (s, 1H), 6.79 (dd, J=1.6, 9.1 Hz, 1H), 6.55 (d, J=8.0 Hz, 1H), 5.42 (d, J=18.6 Hz, 4H), 4.22 (br d, J=2.5 Hz, 1H), 3.96 (s, 3H), 3.83 (br d, J=13.1 Hz, 1H), 3.69-3.52 (m, 2H), 3.26-3.15 (m, 1H), 2.96-2.84 (m, 1H), 2.75-2.64 (m, 1H), 1.43 (s, 10H), 1.22 (d, J=6.6 Hz, 3H)

### Preparation of tert-butyl (2S)-4-(3-(2,6-dioxopiperidin-3-yl)-1-methyl-1H-indazol-6-yl)-2-methylpiperazine-1-carboxylate

To a solution of tert-butyl (S)-4-(3-(2,6-bis(benzyloxy)pyridin-3-yl)-1-methyl-1H-indazol-6-yl)-2-methylpiperazine-1-carboxylate (15.0 g, 24.2 mmol, 1.00 equiv.) in EtOH (150 mL) and THF (150 mL) was added Pd/C (3.70 g, 3.48 mmol, 10.0% purity), Pd(OH)₂ (3.70 g, 26.3 mmol, 1.09 equiv.), and AcOH (15.0 mL) under N₂. The suspension was degassed under vacuum and purged with H₂ three times. The mixture was stirred under H₂ (50 psi) at 50° C. for 4 h. The reaction mixture was filtered, washed with THF (200 mL), and concentrated under reduced pressure to give tert-butyl (2S)-4-(3-(2,6-dioxopiperidin-3-yl)-1-methyl-1H-indazol-6-yl)-2-methylpiperazine-1-carboxylate (9.00 g, 16.9 mmol) as a yellow solid.

LC-MS (ESI): m/z=442.4 [M+H]⁺

### Preparation of 3-(1-methyl-6-((S)-3-methylpiperazin-1-yl)-1H-indazol-3-yl)piperidine-2,6-dione

A mixture of tert-butyl (2S)-4-(3-(2,6-dioxopiperidin-3-yl)-1-methyl-1H-indazol-6-yl)-2-methylpiperazine-1-carboxylate (9.00 g, 20.3 mmol, 1.00 equiv.) in HCl/dioxane (90.0 mL) was stirred at 25° C. for 1 h. The reaction mixture was filtered and concentrated under reduced pressure to give 3-(1-methyl-6-((S)-3-methylpiperazin-1-yl)-1H-indazol-3-yl)piperidine-2,6-dione (5.50 g, 14.3 mmol) as a yellow solid.

LC-MS (ESI): m/z=342.1 [M+H]⁺

¹H NMR: (400 MHz, DMSO-d₆) δ 10.86 (s, 1H), 9.75-9.39 (m, 2H), 7.56 (d, J=8.9 Hz, 1H), 7.02-6.93 (m, 2H), 4.28 (br dd, J=5.0, 9.4 Hz, 1H), 3.92 (s, 3H), 3.89-3.79 (m, 2H), 3.46-3.29 (m, 2H), 3.12 (br d, J=8.6 Hz, 2H), 2.90 (br dd, J=10.9, 12.6 Hz, 1H), 2.70-2.55 (m, 3H), 2.38-2.26 (m, 1H), 2.22-2.09 (m, 1H), 1.35 (d, J=6.5 Hz, 3H)

### Preparation of tert-butyl (3S)-3-(((2S)-4-(3-(2,6-dioxopiperidin-3-yl)-1-methyl-1H-indazol-6-yl)-2-methylpiperazin-1-yl)methyl)pyrrolidine-1-carboxylate

A mixture of tert-butyl (R)-3-formylpyrrolidine-1-carboxylate (305 mg, 1.53 mmol), 3-(1-methyl-6-((S)-3-methylpiperazin-1-yl)-1H-indazol-3-yl)piperidine-2,6-dione (348 mg, 1.02 mmol), and potassium acetate (300 mg, 3.06 mmol) was suspended in THF (4.00 mL) and DMF (0.600 mL).

The reaction mixture was stirred at room temperature for 5 min. The pale beige mixture was then treated all at once with sodium triacetoxyborohydride (324 mg, 1.53 mmol). The reaction mixture allowed to stir at room temperature for 15 min. The reaction mixture was treated with saturated aqueous ammonium chloride solution (5 mL), then extracted with ethyl acetate (3×). The combined organics were dried over sodium sulfate, filtered, and concentrated under reduced pressure. The residue was purified by column chromatography (0-100% (3:1 Ethyl acetate/EtOH)/heptane) to afford tert-butyl (3S)-3-(((2S)-4-(3-(2,6-dioxopiperidin-3-yl)-1-methyl-1H-indazol-6-yl)-2-methylpiperazin-1-yl)methyl)pyrrolidine-1-carboxylate (20.7 mg). LC-MS (ESI): m/z=525.3 [M+H]⁺.

### Preparation of 3-(1-methyl-6-((S)-3-methyl-4-(((R)-pyrrolidin-3-yl)methyl)piperazin-1-yl)-1H-indazol-3-yl)piperidine-2,6-dione bis(trifluoroacetate)

A solution of tert-butyl (3S)-3-(((2S)-4-(3-(2,6-dioxopiperidin-3-yl)-1-methyl-1H-indazol-6-yl)-2-methylpiperazin-1-yl)methyl)pyrrolidine-1-carboxylate (20.2 mg, 1.00 equiv., 38.5 mol) in DCM (0.500 mL) was treated with a solution of TFA (740 mg, 0.500 mL, 169 equiv., 6.49 mmol) and DCM (0.500 mL). The mixture was stirred at room temperature for 1 h. The volatiles were removed under reduced pressure to afford 3-(1-methyl-6-((S)-3-methyl-4-(((R)-pyrrolidin-3-yl)methyl)piperazin-1-yl)-1H-indazol-3-yl)piperidine-2,6-dione bis(trifluoroacetate) (25.1 mg).

LC-MS (ESI): m/z=425.3 [M+H]⁺.

### Preparation of 3-(6-((S)-4-(((S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)pyrrolidin-3-yl)methyl)-3-methylpiperazin-1-yl)-1-methyl-1H-indazol-3-yl)piperidine-2,6-dione

A solution of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (23.4 mg, 50.0 mol), 3-(1-methyl-6-((S)-3-methyl-4-(((R)-pyrrolidin-3-yl)methyl)piperazin-1-yl)-1H-indazol-3-yl)piperidine-2,6-dione bis(trifluoroacetate) (25.1 mg, 38.5 mol), CsF (47.6 mg, 8.15 equiv., 313 mol), and DIEA (19.9 mg, 26.8 μL, 4.00 equiv., 154 mol) in DMSO (0.500 mL) was warmed to 80° C. for 17 h. The reaction mixture was cooled to room temperature, filtered, and purified by prep-HPLC to afford 3-(6-((S)-4-(((S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)pyrrolidin-3-yl)methyl)-3-methylpiperazin-1-yl)-1-methyl-1H-indazol-3-yl)piperidine-2,6-dione (13.0 mg).

¹H NMR (400 MHz, DMSO-d₆) δ 10.84 (s, 1H), 8.67 (s, 1H), 8.23 (s, 1H), 8.03 (s, 1H), 7.87 (d, J=8.3 Hz, 1H), 7.49 (d, J=9.0 Hz, 1H), 7.42 (d, J=9.1 Hz, 1H), 6.91 (d, J=9.3 Hz, 1H), 6.83 (s, 1H), 6.19-6.05 (m, 1H), 4.54-4.31 (m, 2H), 4.26 (dd, J=9.1, 5.1 Hz, 1H), 3.90 (s, 3H), 3.71-3.56 (m, 2H), 3.55-3.45 (m, 6H), 3.29 (s, 3H), 3.10-2.82 (m, 2H), 2.81-2.56 (m, 2H), 2.57-2.53 (m, 2H), 2.37-2.24 (m, 2H), 2.23-2.11 (m, 2H), 2.06-1.93 (m, 1H), 1.88 (s, 1H), 1.78-1.63 (m, 1H), 1.41-1.29 (m, 1H), 1.14-0.99 (m, 3H), 0.81-0.65 (m, 1H), 0.57-0.47 (m, 2H), 0.43-0.27 (m, 1H). LC-MS (ESI): m/z=856.4 [M+H]⁺.

### Example 235. 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-5-fluoro-1-methyl-1H-indazol-3-yl)piperidine-2,6-dione (Compound 563a)

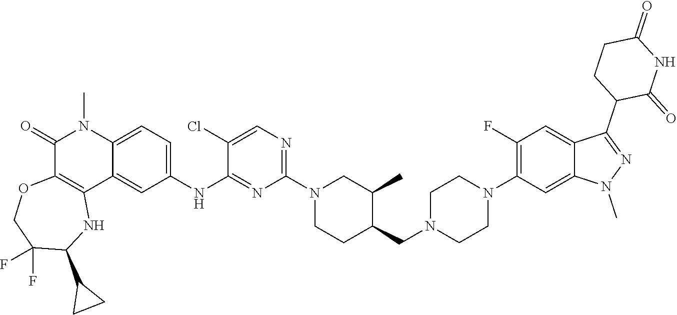 [SMILES: C[C@@H]1CN(c2ncc(Cl)c(Nc3ccc4c(c3)c3c(c(=O)n4C)OCC(F)(F)[C@H](C4CC4)N3)n2)CC[C@@H]1CN1CCN(c2cc3c(cc2F)c(C2CCC(=O)NC2=O)nn3C)CC1]

### Preparation of tert-butyl 4-(3-(2,6-bis(benzyloxy)pyridin-3-yl)-5-fluoro-1-methyl-1H-indazol-6-yl)piperazine-1-carboxylate

A mixture of tert-butyl piperazine-1-carboxylate (548 mg, 2.94 mmol), 3-(2,6-bis(benzyloxy)pyridin-3-yl)-6-bromo-5-fluoro-1-methyl-1H-indazole (1.00 g, 1.93 mmol), and RuPhos-Pd-G3 (170 mg, 203 mol) in dioxane (12.00 mL) was sparged with N₂ for 5 min. The mixture was treated with NaOtBu (1.93 mL, 2.00 molar, 3.86 mmol) and sparged again with N₂ for another 5 min. The mixture was then warmed to 100° C. for 1 h. The reaction mixture was purified directly by column chromatography (0-100% (3:1 Ethyl acetate/EtOH)/heptane) to afford tert-butyl 4-(3-(2,6-bis(benzyloxy)pyridin-3-yl)-5-fluoro-1-methyl-1H-indazol-6-yl)piperazine-1-carboxylate (1.063 g) as an orange solid. ¹H NMR (400 MHz, DMSO) δ 7.91 (d, J=8.1 Hz, 1H), 7.48 (d, J=7.2 Hz, 3H), 7.39 (q, J=7.0 Hz, 4H), 7.34-7.27 (m, 3H), 7.14 (d, J=7.2 Hz, 1H), 6.58 (d, J=8.1 Hz, 1H), 5.45 (d, J=9.4 Hz, 4H), 4.01 (s, 3H), 3.52 (s, 5H), 3.00 (s, 5H), 1.43 (s, 11H). LC-MS (ESI): m/z=534.3 [M-Bn+H]⁺.

### Preparation of 6-(benzyloxy)-3-(5-fluoro-1-methyl-6-(piperazin-1-yl)-1H-indazol-3-yl)pyridin-2-ol bis(trifluoroacetate)

A solution of tert-butyl 4-(3-(2,6-bis(benzyloxy)pyridin-3-yl)-5-fluoro-1-methyl-1H-indazol-6-yl)piperazine-1-carboxylate (330 mg, 29 mol) in DCM (2.00 mL) was treated with a 1:1 solution of DCM (2.00 mL) and TFA (2.00 mL, 26.0 mmol). The mixture was stirred for 2 hours at room temperature. The reaction was concentrated under reduced pressure to afford 6-(benzyloxy)-3-(5-fluoro-1-methyl-6-(piperazin-1-yl)-1H-indazol-3-yl)pyridin-2-ol bis(trifluoroacetate) (351 mg), which was used as is in the next step. LC-MS (ESI): m/z=434.3 [M+H]⁺

### Preparation of tert-butyl (3S, 4S)-4-(4-(3-(6-(benzyloxy)-2-hydroxypyridin-3-yl)-5-fluoro-1-methyl-1H-indazol-6-yl)piperazine-1-carbonyl)-3-methylpiperidine-1-carboxylate

A solution of (3S, 4S)-1-(tert-butoxycarbonyl)-3-methylpiperidine-4-carboxylic acid (100 mg, 411 mol), 6-(benzyloxy)-3-(5-fluoro-1-methyl-6-(piperazin-1-yl)-1H-indazol-3-yl)pyridin-2-ol bis(trifluoroacetate) (234 mg, 354 mol), and HATU (174 mg, 458 mol) in DMA (2.40 mL) was treated with DIEA (368 μL, 2.11 mmol). The mixture was stirred at room temperature for 2 h. The volatiles were removed under reduced pressure and purified by column chromatography (0-50% (3:1 Ethyl acetate/EtOH)/heptane) to afford tert-butyl (3S, 4S)-4-(4-(3-(6-(benzyloxy)-2-hydroxypyridin-3-yl)-5-fluoro-1-methyl-1H-indazol-6-yl)piperazine-1-carbonyl)-3-methylpiperidine-1-carboxylate (181 mg). LC-MS (ESI): m/z=659.4 [M+H]⁺

### Preparation of tert-butyl (3S, 4S)-4-((4-(3-(6-(benzyloxy)-2-hydroxypyridin-3-yl)-5-fluoro-1-methyl-1H-indazol-6-yl)piperazin-1-yl)methyl)-3-methylpiperidine-1-carboxylate

A solution of tert-butyl (3S, 4S)-4-(4-(3-(6-(benzyloxy)-2-hydroxypyridin-3-yl)-5-fluoro-1-methyl-1H-indazol-6-yl)piperazine-1-carbonyl)-3-methylpiperidine-1-carboxylate (180 mg, 273 mol) in THF (1.50 mL) was treated dropwise with BH₃·DMS (259 μL, 2.73 mmol). The mixture was stirred at room temperature for 6 h, warmed to 50° C., and stirred for 2 h. The reaction mixture was treated dropwise with MeOH (2 mL) and warmed to 70° C. After 13 h, the reaction mixture was cooled to room temperature. The volatiles were removed under reduced pressure to afford tert-butyl (3S, 4S)-4-((4-(3-(6-(benzyloxy)-2-hydroxypyridin-3-yl)-5-fluoro-1-methyl-1H-indazol-6-yl)piperazin-1-yl)methyl)-3-methylpiperidine-1-carboxylate (166 mg), which was used as is without further purification. LC-MS (ESI): m/z=645.4 [M+H]⁺.

### Preparation of tert-butyl (3S, 4S)-4-((4-(3-(2,6-dioxopiperidin-3-yl)-5-fluoro-1-methyl-1H-indazol-6-yl)piperazin-1-yl)methyl)-3-methylpiperidine-1-carboxylate

A solution of tert-butyl (3S, 4S)-4-((4-(3-(6-(benzyloxy)-2-hydroxypyridin-3-yl)-5-fluoro-1-methyl-1H-indazol-6-yl)piperazin-1-yl)methyl)-3-methylpiperidine-1-carboxylate (164 mg, 223 mol) in iPrOH (4.00 mL) and DMF (0.800 mL) was sparged with N₂ for 5 min. To this mixture was added Pd(OH)₂ (62.7 mg, 20% Wt, 89.3 mol). The resulting mixture was sparged with H₂ for 5 min. The reaction mixture was warmed to 50° C. under an H₂ atmosphere for 5 h. The reaction mixture was diluted with THF (1.60 mL), charged with additional Pd(OH)₂ (62.7 mg, 20% Wt, 89.3 mol), and sparged with H₂ for 5 min. The mixture was stirred at 50° C. under H₂ atmosphere for 16 h. The reaction mixture was filtered through a pad of celite and rinsed thoroughly with DMF, methanol, and DCM. The filtrate was concentrated under reduced pressure to afford tert-butyl (3S, 4S)-4-((4-(3-(2,6-dioxopiperidin-3-yl)-5-fluoro-1-methyl-1H-indazol-6-yl)piperazin-1-yl)methyl)-3-methylpiperidine-1-carboxylate (159 mg). LC-MS (ESI): m/z=557.3 [M+H]⁺.

### Preparation of 3-(5-fluoro-1-methyl-6-(4-(((3S, 4S)-3-methylpiperidin-4-yl)methyl)piperazin-1-yl)-1H-indazol-3-yl)piperidine-2,6-dione

A solution of tert-butyl (3S, 4S)-4-((4-(3-(2,6-dioxopiperidin-3-yl)-5-fluoro-1-methyl-1H-indazol-6-yl)piperazin-1-yl)methyl)-3-methylpiperidine-1-carboxylate (124 mg, 223 mol) and DCM (1.00 mL) was treated with a 1:1 solution of TFA (1.00 mL) and DCM (1.00 mL). The mixture was stirred at room temperature for 2 h. The volatiles were removed under reduced pressure, and the residue was suspended in DCM (2 mL). The volatiles removed again under reduced pressure to afford 3-(5-fluoro-1-methyl-6-(4-(((3S, 4S)-3-methylpiperidin-4-yl)methyl)piperazin-1-yl)-1H-indazol-3-yl)piperidine-2,6-dione (102 mg). LC-MS (ESI): m/z=457.4 [M+H]⁺.

### Preparation of 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-5-fluoro-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (136 mg, 290 mol), 3-(5-fluoro-1-methyl-6-(4-(((3S, 4S)-3-methylpiperidin-4-yl)methyl)piperazin-1-yl)-1H-indazol-3-yl)piperidine-2,6-dione (102 mg, 223 mol), and CsF (136 mg, 894 mol) in DMSO (1.00 mL) was treated with DIEA (233 μL, 1.34 mmol) and warmed to 80° C. for 16 h. The reaction mixture was cooled to room temperature, filtered, and purified by prep-HPLC to afford 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-5-fluoro-1-methyl-1H-indazol-3-yl)piperidine-2,6-dione (43.5 mg). 1H NMR (400 MHz, DMSO-d₆) δ 10.85 (s, 1H), 8.71 (s, 1H), 8.14 (s, 1H), 8.01 (s, 1H), 7.83-7.67 (m, 1H), 7.45 (d, J=10.6 Hz, 2H), 7.10 (d, J=7.0 Hz, 1H), 6.20 (s, 1H), 4.49-4.29 (m, 2H), 4.29-4.18 (m, 1H), 3.94 (s, 3H), 3.74-3.59 (m, 1H), 3.57 (s, 3H), 3.20-2.93 (m, 6H), 2.92-2.77 (m, 1H), 2.67-2.52 (m, 4H), 2.25-2.03 (m, 3H), 1.98-1.83 (m, 1H), 1.58-1.38 (m, 4H), 1.38-1.19 (m, 3H), 0.83-0.63 (m, 4H), 0.62-0.46 (m, 3H), 0.39-0.27 (m, 1H). LC-MS (ESI): m/z=444.8 [M+2H]²+.

### Example 236. 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-5-fluoro-1-methyl-1H-indazol-3-yl)piperidine-2,6-dione (Compound 563b)

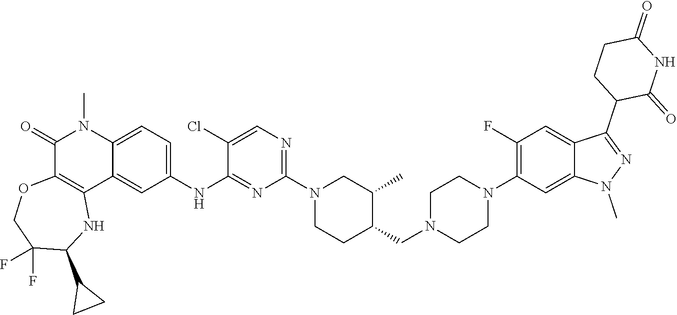 [SMILES: C[C@H]1CN(c2ncc(Cl)c(Nc3ccc4c(c3)c3c(c(=O)n4C)OCC(F)(F)[C@H](C4CC4)N3)n2)CC[C@H]1CN1CCN(c2cc3c(cc2F)c(C2CCC(=O)NC2=O)nn3C)CC1]

### Preparation of 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-5-fluoro-1-methyl-1H-indazol-3-yl)piperidine-2,6-dione

This compound was prepared in a manner analogous to that described for Example 235 to afford 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-methylpiperidin-4-yl)methyl)piperazin-1-yl)-5-fluoro-1-methyl-1H-indazol-3-yl)piperidine-2,6-dione (13.8 mg).

¹H NMR (400 MHz, DMSO-d₆) δ 10.85 (s, 1H), 8.73 (s, 1H), 8.14 (s, 1H), 8.00 (s, 1H), 7.83-7.67 (m, 1H), 7.45 (d, J=9.4 Hz, 2H), 7.10 (d, J=7.1 Hz, 1H), 6.51 (s, 1H), 4.49-4.29 (m, 2H), 4.31-4.16 (m, 1H), 3.95 (s, 3H), 3.74-3.59 (m, 1H), 3.54 (s, 3H), 3.17-2.90 (m, 6H), 2.94-2.79 (m, 1H), 2.69-2.55 (m, 4H), 2.20-2.00 (m, 3H), 1.99-1.85 (m, 1H), 1.59-1.36 (m, 4H), 1.38-1.19 (m, 3H), 0.83-0.63 (m, 4H), 0.62-0.46 (m, 3H), 0.39-0.27 (m, 1H). LC-MS (ESI): m/z=444.9 [M+2H]²+.

### Example 237: 3-(7-((R)-4-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)-3-methylpiperazin-1-yl)-1-methyl-1H-indazol-3-yl)piperidine-2,6-dione (Compound 493b)

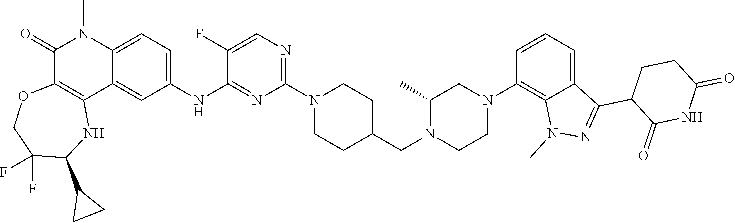 [SMILES: C[C@@H]1CN(c2cccc3c(C4CCC(=O)NC4=O)nn(C)c23)CCN1CC1CCN(c2ncc(F)c(Nc3ccc4c(c3)c3c(c(=O)n4C)OCC(F)(F)[C@H](C4CC4)N3)n2)CC1]

### Preparation of benzyl (R)-4-((1-(tert-butoxycarbonyl)piperidin-4-yl)methyl)-3-methylpiperazine-1-carboxylate

To a stirred solution of benzyl (R)-3-methylpiperazine-1-carboxylate (2036.3 mg, 1.00 equiv., 8.69 mmol) and tert-butyl 4-formylpiperidine-1-carboxylate (1909.2 mg, 1.03 equiv., 8.95 mmol) in MeOH (20.00 mL) was added decaborane (318.6 mg, 0.34 mL, 0.300 equiv., 2.61 mmol). The reaction mixture was stirred at room temperature overnight. The reaction mixture was evaporated to dryness, absorbed onto silica, and purified via flash column chromatography using a gradient (0-100% (A: heptane; B: 3:1 ethyl acetate/EtOH)) to afford benzyl (R)-4-((1-(tert-butoxycarbonyl)piperidin-4-yl)methyl)-3-methylpiperazine-1-carboxylate (918.2 mg, 2.13 mmol). LC-MS (ESI): m/z=432.5 [M+H]⁺.

### Preparation of tert-butyl (R)-4-((2-methylpiperazin-1-yl)methyl)piperidine-1-carboxylate

To a solution of benzyl (R)-4-((1-(tert-butoxycarbonyl)piperidin-4-yl)methyl)-3-methylpiperazine-1-carboxylate (3.014 g, 1.00 equiv., 6.984 mmol) in i-PrOH (13.0 mL), degassed with nitrogen, was added palladium(2+) dihydroxide (980.7 mg, 20% Wt, 0.20 equiv., 1.397 mmol). Hydrogen gas was then bubbled through the solution. After 18 hours, the reaction mixture was filtered through celite, then added into water. A dark residue was removed by vacuum filtration, and the filtrate was evaporated to dryness to afford tert-butyl (R)-4-((2-methylpiperazin-1-yl)methyl)piperidine-1-carboxylate (905.9 mg, 6.984 mmol). LC-MS (ESI): m/z=298.4 [M+H]⁺.

### Preparation of tert-butyl (R)-4-((4-(3-(2,6-bis(benzyloxy)pyridin-3-yl)-1-methyl-1H-indazol-7-yl)-2-methylpiperazin-1-yl)methyl)piperidine-1-carboxylate

Dry nitrogen was bubbled through a mixture of tert-butyl (R)-4-((2-methylpiperazin-1-yl)methyl)piperidine-1-carboxylate (906 mg, 1 equiv., 3.05 mmol), 3-(2,6-bis(benzyloxy)pyridin-3-yl)-7-bromo-1-methyl-1H-indazole (1.83 g, 1.2 equiv., 3.66 mmol), and RuPhos-Pd-G3 (196 mg, 0.077 equiv., 235 mol) in THF (22.0 mL) for 10 minutes. While still sparging the solution with N₂(g), a solution of sodium tert-butoxide (849 mg, 4.42 mL, 2.0 molar, 2.9 equiv., 8.83 mmol) was added. The reaction mixture was warmed to 70° C. and stirred. After 2 hours, the reaction mixture was diluted into ethyl acetate, washed with water (3×), then extracted once with ethyl acetate. The organic residue was purified via reverse phase flash column chromatography (10-100% (A: water+0.1% formic acid; B: MeCN+0.1% formic acid)) to afford tert-butyl (R)-4-((4-(3-(2,6-bis(benzyloxy)pyridin-3-yl)-1-methyl-1H-indazol-7-yl)-2-methylpiperazin-1-yl)methyl)piperidine-1-carboxylate (2.08 g, 2.90 mmol). LC-MS (ESI): m/z=717.6 [M+H]⁺.

### Preparation of 3-(1-methyl-7-((R)-3-methyl-4-(piperidin-4-ylmethyl)piperazin-1-yl)-1H-indazol-3-yl)piperidine-2,6-dione

To a solution of tert-butyl (R)-4-((4-(3-(2,6-bis(benzyloxy)pyridin-3-yl)-1-methyl-1H-indazol-7-yl)-2-methylpiperazin-1-yl)methyl)piperidine-1-carboxylate (1.617 g, 1 equiv., 3.002 mmol) in DCM (10.0 mL)/iPrOH (10.0 mL) was added HCl (4.0 M in dioxane) (437.8 mg, 3.002 mL, 4 M, 4 equiv., 12.01 mmol). The mixture was stirred overnight at room temperature. The resulting heterogeneous mixture was diluted with ethyl acetate and washed with water.

The aqueous layer was frozen, then dried via lyophilization to afford 3-(1-methyl-7-((R)-3-methyl-4-(piperidin-4-ylmethyl)piperazin-1-yl)-1H-indazol-3-yl)piperidine-2,6-dione (1046 mg, 1.98 mmol). LC-MS (ESI): m/z=439.3[M+H]⁺.

### Preparation of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one

To a solution of (S)-10-amino-2-cyclopropyl-3,3-difluoro-7-methyl-1,2,3,4-tetrahydro-[1,4]oxazepino[2,3-c]quinolin-6(7H)-one (250 mg, 1.00 equiv., 778 mol) and 2,4-dichloro-5-fluoropyrimidine (143 mg, 1.10 equiv., 856 mol) in DMSO (3 mL) was added DIEA (402 mg, 542 μL, 4.00 equiv., 3.11 mmol). The reaction mixture was warmed to 80° C. for 4 h. The reaction mixture was cooled to room temperature and then directly purified via reverse phase flash column chromatography (10-100% (A: water+0.1% formic acid; B: MeCN+0.1% formic acid)) to afford (S)-10-((2-chloro-5-fluoropyrimidin-4-yl)amino)-2-cyclopropyl-3,3-difluoro-7-methyl-1,2,3,4-tetrahydro-[1,4]oxazepino [2,3-c]quinolin-6(7H)-one (243.2 mg, 778 μmol). LC-MS (ESI): m/z=452.3 [M+H]⁺.

### Preparation of 3-(7-((R)-4-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)-3-methylpiperazin-1-yl)-1-methyl-1H-indazol-3-yl)piperidine-2,6-dione

To a mixture of (S)-10-((2-chloro-5-fluoropyrimidin-4-yl)amino)-2-cyclopropyl-3,3-difluoro-7-methyl-1,2,3,4-tetrahydro-[1,4]oxazepino [2,3-c]quinolin-6(7H)-one (55.1 mg, 1.5 equiv., 122 mol), 3-(1-methyl-7-((R)-3-methyl-4-(piperidin-4-ylmethyl)piperazin-1-yl)-1H-indazol-3-yl)piperidine-2,6-dione, 2.5HCl (43.1 mg, 1.00 equiv., 81.4 mol), and CsF (37.1 mg, 3.00 equiv., 244 mol) in DMSO (2.00 mL) was added DIEA (52.6 mg, 70.9 μL, 5.00 equiv., 407 mol). The reaction mixture was warmed to 80° C. and stirred for 16 hours. The reaction mixture was cooled to room temperature and then directly purified via prep-HPLC to afford 3-(7-((R)-4-((1-(4-(((S)-2-cyclopropyl-3,3-difluoro-7-methyl-6-oxo-1,2,3,4,6,7-hexahydro-[1,4]oxazepino [2,3-c]quinolin-10-yl)amino)-5-fluoropyrimidin-2-yl)piperidin-4-yl)methyl)-3-methylpiperazin-1-yl)-1-methyl-1H-indazol-3-yl)piperidine-2,6-dione (13.9 mg, 15 μmol). ¹H NMR (400 MHz, DMSO) δ 10.87 (s, 1H), 9.26 (s, 1H), 8.22 (s, 1H), 8.02-7.97 (m, 1H), 7.81-7.75 (m, 1H), 7.49-7.42 (m, 1H), 7.06-6.92 (m, 2H), 5.99 (s, 1H), 4.53-4.28 (m, 6H), 4.24 (s, 3H), 3.56 (s, 3H), 3.22-2.93 (m, 2H), 2.93-2.72 (m, 3H), 2.72-2.56 (m, 2H), 2.33 (s, 2H), 2.22-2.10 (m, 1H), 2.04-1.55 (m, 4H), 1.35 (s, 1H), 1.02 (s, 5H), 0.80-0.64 (m, 1H), 0.53 (s, 2H), 0.38 (s, 1H), 0.10-0.04 (m, 2H). LC-MS (ESI): m/z=854.7 [M+H]⁺.

### Example 238: 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)-3-methylpiperazin-1-yl)-1-methyl-1H-indazol-3-yl)piperidine-2,6-dione (Compound 496b)

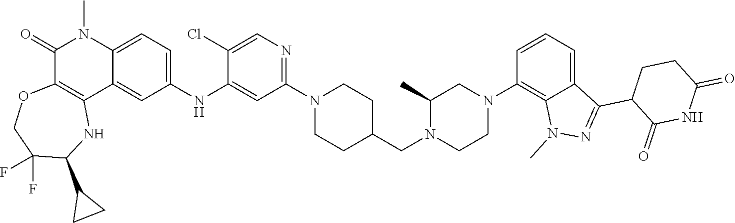 [SMILES: C[C@H]1CN(c2cccc3c(C4CCC(=O)NC4=O)nn(C)c23)CCN1CC1CCN(c2cc(Nc3ccc4c(c3)c3c(c(=O)n4C)OCC(F)(F)[C@H](C4CC4)N3)c(Cl)cn2)CC1]

### Preparation of (S)-10-((2-(4-(1,3-dioxolan-2-yl)piperidin-1-yl)-5-chloropyridin-4-yl)amino)-2-cyclopropyl-3,3-difluoro-7-methyl-1,2,3,4-tetrahydro-[1,4]oxazepino[2,3-c]quinolin-6(7H)-one

A mixture of (S)-10-amino-2-cyclopropyl-3,3-difluoro-7-methyl-1,2,3,4-tetrahydro-[1,4]oxazepino[2,3-c]quinolin-6(7H)-one (930.2 mg, 1.042 equiv., 2.895 mmol), 2-(4-(1,3-dioxolan-2-yl)piperidin-1-yl)-5-chloro-4-iodopyridine (1096.4 mg, 1.00 equiv., 2.7782 mmol), and BrettPhos-Pd-G3 (251.85 mg, 0.10 equiv., 277.82 mol) in 1,4-dioxane (20.00 mL) was sparged with N₂(g) for 15 min. The reaction mixture was then treated with NaOtBu (2.0 M in THF, 801.0 mg, 4.1673 mL, 2.00 M, 3.00 equiv., 8.3347 mmol). The reaction mixture was then warmed to 100° C. and stirred for 1 hour. The reaction was diluted into ethyl acetate and washed with water (3×). The organic layer was concentrated under reduced pressure then purified via flash column chromatography (0-100% ethyl acetate/heptanes) to afford (S)-10-((2-(4-(1,3-dioxolan-2-yl)piperidin-1-yl)-5-chloropyridin-4-yl)amino)-2-cyclopropyl-3,3-difluoro-7-methyl-1,2,3,4-tetrahydro-[1,4]oxazepino[2,3-c]quinolin-6(7H)-one. LC-MS (ESI): m/z=588.4 [M+H]⁺.

### Preparation of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde

(S)-10-((2-(4-(1,3-dioxolan-2-yl)piperidin-1-yl)-5-chloropyridin-4-yl)amino)-2-cyclopropyl-3,3-difluoro-7-methyl-1,2,3,4-tetrahydro-[1,4]oxazepino[2,3-c]quinolin-6(7H)-one (387.5 mg, 1 equiv., 659.0 mol) was dissolved in formic acid (1500 μL). The mixture was then heated to 80° C. and stirred for 2 hours. After 2 hours, the reaction mixture was concentrated under reduced pressure to afford (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde.

LC-MS (ESI): m/z=544.1 [M+H]⁺.

### Preparation of tert-butyl (S)-4-(3-(2,6-bis(benzyloxy)pyridin-3-yl)-1-methyl-1H-indazol-7-yl)-2-methylpiperazine-1-carboxylate

A solution of tert-butyl (S)-2-methylpiperazine-1-carboxylate (2.161 g, 1.8 equiv., 10.79 mmol), 3-(2,6-bis(benzyloxy)pyridin-3-yl)-7-bromo-1-methyl-1H-indazole (3000 mg, 1 equiv., 5.995 mmol), and RuPhos-Pd-G3 (351 mg, 0.0700 equiv., 420 mol) in THF (30.0 mL) was degassed with nitrogen. Sodium tert-butoxide in THF (1.671 g, 8.693 mL, 2.0 M, 2.9 equiv., 17.39 mmol) was added. The reaction mixture was warmed at 70° C. and stirred for 1.5 hours. The reaction mixture was filtered through celite and then purified via flash column (0-80% ethyl acetate/heptanes) to afford tert-butyl (S)-4-(3-(2,6-bis(benzyloxy)pyridin-3-yl)-1-methyl-1H-indazol-7-yl)-2-methylpiperazine-1-carboxylate. LC-MS (ESI): m/z=620.4 [M+H]⁺.

### Preparation of tert-butyl (2S)-4-(3-(2,6-dioxopiperidin-3-yl)-1-methyl-1H-indazol-7-yl)-2-methylpiperazine-1-carboxylate

A solution of tert-butyl (S)-4-(3-(2,6-bis(benzyloxy)pyridin-3-yl)-1-methyl-1H-indazol-7-yl)-2-methylpiperazine-1-carboxylate (3.500 g, 1 equiv., 5.647 mmol) in isopropanol (6.00 mL)/DMF (1.20 mL)/ethyl acetate (1.20 mL) was treated with hydrogen and Pd(OH)₂/C (0.99 g, 0.25 equiv.).

After 2 hours, the reaction mixture was filtered through celite, and evaporated to dryness to afford tert-butyl (2S)-4-(3-(2,6-dioxopiperidin-3-yl)-1-methyl-1H-indazol-7-yl)-2-methylpiperazine-1-carboxylate. LC-MS (ESI): m/z=442.3 [M+H]⁺.

### Preparation of 3-(1-methyl-7-((S)-3-methylpiperazin-1-yl)-1H-indazol-3-yl)piperidine-2,6-dione, 2HCl

tert-Butyl (2S)-4-(3-(2,6-dioxopiperidin-3-yl)-1-methyl-1H-indazol-7-yl)-2-methylpiperazine-1-carboxylate (3.8595 g, 1 equiv., 5.8406 mmol) was dissolved in DCM (40 mL) and treated with HCl in dioxane (1.704 g, 11.681 mL, 4 M, 8 equiv., 46.724 mmol). The mixture was stirred at room temperature for 18 hours. The reaction mixture was concentrated, then triturated with hot stirring ethyl acetate for 1 hour. The mixture was cooled to room temperature, filtered, and dried under vacuum to afford 3-(1-methyl-7-((S)-3-methylpiperazin-1-yl)-1H-indazol-3-yl)piperidine-2,6-dione, 2HCl. LC-MS (ESI): m/z=342.1 [M+H]⁺.

### Preparation of 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)-3-methylpiperazin-1-yl)-1-methyl-1H-indazol-3-yl)piperidine-2,6-dione

A mixture of (S)-1-(5-chloro-4-((2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidine-4-carbaldehyde (227 mg, 1 equiv., 417 mol), 3-(1-methyl-7-((S)-3-methylpiperazin-1-yl)-1H-indazol-3-yl)piperidine-2,6-dione, 2HCl (173 mg, 1 equiv., 417 mol), and potassium acetate (246 mg, 6.00 equiv., 2.50 mmol) was suspended in THF (10 mL) and DMF (1.5 mL). The mixture was then stirred at room temperature for 40 minutes. The heterogeneous mixture was then treated with sodium triacetoxyborohydride (133 mg, 1.50 equiv., 626 mol). After 18 hours, the reaction mixture was diluted with DMSO, filtered, and purified via prep-HPLC to afford 3-(7-((S)-4-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)piperidin-4-yl)methyl)-3-methylpiperazin-1-yl)-1-methyl-1H-indazol-3-yl)piperidine-2,6-dione. ¹H NMR (400 MHz, DMSO-d6) δ 10.87 (s, 1H), 8.12-8.05 (m, 1H), 8.00 (s, 1H), 7.92 (s, 1H), 7.54-7.43 (m, 2H), 7.42-7.35 (m, 1H), 7.06-6.98 (m, 2H), 6.44-6.39 (m, 1H), 6.10 (s, 1H), 4.52-4.29 (m, 3H), 4.24 (s, 3H), 4.06-3.92 (m, 2H), 3.58 (s, 3H), 3.29-2.74 (m, 5H), 2.74-2.55 (m, 2H), 2.41-2.24 (m, 1H), 2.22-2.12 (m, 1H), 2.04-1.53 (m, 2H), 1.39-0.92 (m, 9H), 0.90-0.80 (m, 1H), 0.76-0.67 (m, 1H), 0.55-0.48 (m, 2H), 0.39-0.25 (m, 1H). LC-MS (ESI): m/z=869.1 [M+H]⁺.

### Example 239: 3-(7-((S)-4-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)-3-methylpiperazin-1-yl)-1-methyl-1H-indazol-3-yl)piperidine-2,6-dione (Compound 493a)

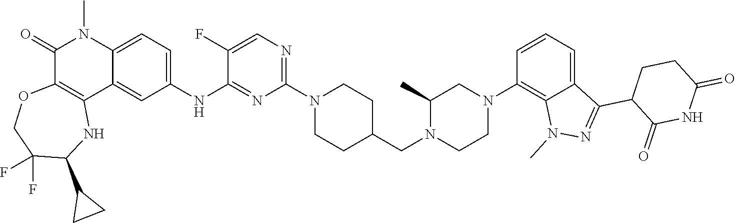 [SMILES: C[C@H]1CN(c2cccc3c(C4CCC(=O)NC4=O)nn(C)c23)CCN1CC1CCN(c2ncc(F)c(Nc3ccc4c(c3)c3c(c(=O)n4C)OCC(F)(F)[C@H](C4CC4)N3)n2)CC1]

### Preparation of benzyl (S)-4-((1-(tert-butoxycarbonyl)piperidin-4-yl)methyl)-3-methylpiperazine-1-carboxylate

Benzyl (S)-3-methylpiperazine-1-carboxylate (2562 mg, 1.00 equiv., 10.93 mmol) and tert-butyl 4-formylpiperidine-1-carboxylate (2.402 g, 1.03 equiv., 11.26 mmol) were dissolved in MeOH (10.00 mL). To this mixture was added decaborane(14) (334.1 mg, 0.36 mL, 0.250 equiv., 2.734 mmol). The reaction mixture was stirred at room temperature overnight. The mixture was evaporated to dryness, adsorbed onto silica, and purified via flash column chromatography (0-100% B (A: heptane, B: 3:1 ethyl acetate/EtOH)) to afford benzyl (S)-4-((1-(tert-butoxycarbonyl)piperidin-4-yl)methyl)-3-methylpiperazine-1-carboxylate (3.9753 g, 9.2110 mmol). LC-MS (ESI): m/z=432.3 [M+H]⁺.

### Preparation of tert-butyl (S)-4-((2-methylpiperazin-1-yl)methyl)piperidine-1-carboxylate

A flask containing a solution of benzyl (S)-4-((1-(tert-butoxycarbonyl)piperidin-4-yl)methyl)-3-methylpiperazine-1-carboxylate (1.76 g, 1.00 equiv., 4.08 mmol) in i-PrOH (13.0 mL) was evacuated and backfilled with N₂ (2×). The solution was charged with palladium(II) dihydroxide (573 mg, 20% Wt, 0.20 equiv., 816 mol), and the flask was evacuated and backfilled with H₂.

The mixture was stirred for 16 hours at room temperature. The mixture was filtered through a pad of celite and concentrated to give tert-butyl (S)-4-((2-methylpiperazin-1-yl)methyl)piperidine-1-carboxylate (1.4038 g, 4.7196 mmol) as an oil. LC-MS (ESI): m/z=298.4 [M+H]⁺.

### Preparation of tert-butyl (S)-4-((4-(3-(2,6-bis(benzyloxy)pyridin-3-yl)-1-methyl-1H-indazol-7-yl)-2-methylpiperazin-1-yl)methyl)piperidine-1-carboxylate

Dry N₂ was bubbled through a mixture of tert-butyl (S)-4-((2-methylpiperazin-1-yl)methyl)piperidine-1-carboxylate (543 mg, 1 equiv., 1.83 mmol), 3-(2,6-bis(benzyloxy)pyridin-3-yl)-7-bromo-1-methyl-1H-indazole (1.10 g, 1.2 equiv., 2.19 mmol), and RuPhos-Pd-G3 (118 mg, 0.077 equiv., 141 mol) in 1,4-dioxane (15.0 mL) for 10 minutes. While still sparging the solution with N₂, a solution of sodium tert-butoxide in THF (526 mg, 2.74 mL, 2.0 molar, 3 equiv., 5.48 mmol) was added. The reaction mixture was heated to 100° C. and stirred for 1 hour. The mixture was diluted into ethyl acetate, washed with water, and purified via C₁₈ flash column chromatography (10-100% (A: water+0.1% formic acid; B: MeCN+0.1% formic acid)) to afford tert-butyl (S)-4-((4-(3-(2,6-bis(benzyloxy)pyridin-3-yl)-1-methyl-1H-indazol-7-yl)-2-methylpiperazin-1-yl)methyl)piperidine-1-carboxylate. LC-MS (ESI): m/z=717.6 [M+H]⁺.

### Preparation of tert-butyl 4-(((2S)-4-(3-(2,6-dioxopiperidin-3-yl)-1-methyl-1H-indazol-7-yl)-2-methylpiperazin-1-yl)methyl)piperidine-1-carboxylate

A solution of tert-butyl (S)-4-((4-(3-(2,6-bis(benzyloxy)pyridin-3-yl)-1-methyl-1H-indazol-7-yl)-2-methylpiperazin-1-yl)methyl)piperidine-1-carboxylate (303.5 mg, 1 equiv., 423.3 mol) in isopropanol (10.00 mL)/DMF (2.000 mL) was treated with palladium hydroxide on carbon (74.31 mg, 20% Wt, 0.25 equiv., 105.8 mol). The reaction mixture was stirred under hydrogen atmosphere at 50° C. for 16 hours. The mixture was filtered through a pad of celite, and the filtrate was purified via prep-HPLC to afford tert-butyl 4-(((2S)-4-(3-(2,6-dioxopiperidin-3-yl)-1-methyl-1H-indazol-7-yl)-2-methylpiperazin-1-yl)methyl)piperidine-1-carboxylate. LC-MS (ESI): m/z=539.5 [M+H]⁺.

### Preparation of 3-(1-methyl-7-((S)-3-methyl-4-(piperidin-4-ylmethyl)piperazin-1-yl)-1H-indazol-3-yl)piperidine-2,6-dione

A solution of tert-butyl 4-(((2S)-4-(3-(2,6-dioxopiperidin-3-yl)-1-methyl-1H-indazol-7-yl)-2-methylpiperazin-1-yl)methyl)piperidine-1-carboxylate (338.7 mg, 1 equiv., 628.7 mol) in DCM (5.0 mL)/iPrOH (5.0 mL) was treated with HCl (4.0 M in dioxane) (183.4 mg, 1.257 mL, 8 equiv., 5.030 mmol). The mixture was stirred at room temperature for 16 hours. The reaction mixture was evaporated to dryness to afford 3-(1-methyl-7-((S)-3-methyl-4-(piperidin-4-ylmethyl)piperazin-1-yl)-1H-indazol-3-yl)piperidine-2,6-dione (256.4 mg, 484.0 mol). LC-MS (ESI): m/z=439.4 [M+H]⁺.

### Preparation of 3-(7-((S)-4-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)-3-methylpiperazin-1-yl)-1-methyl-1H-indazol-3-yl)piperidine-2,6-dione

A mixture of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (38.9 mg, 1.0 equiv., 86.1 mol), 3-(1-methyl-7-((S)-3-methyl-4-(piperidin-4-ylmethyl)piperazin-1-yl)-1H-indazol-3-yl)piperidine-2,6-dione (45.6 mg, 1.00 equiv., 86.1 mol), and CsF (39.2 mg, 3.00 equiv., 258 mol) in DMSO (2.00 mL) was treated with DIEA (55.6 mg, 75.0 μL, 5.00 equiv., 430 mol). The mixture was warmed to 140° C. and stirred for 1.5 hours. The reaction mixture was diluted further into DMSO, filtered, and then purified via prep-HPLC to afford 3-(7-((S)-4-((1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)piperidin-4-yl)methyl)-3-methylpiperazin-1-yl)-1-methyl-1H-indazol-3-yl)piperidine-2,6-dione (13.9 mg, 15 mol). ¹H NMR (400 MHz, DMSO) δ 10.87 (s, 1H), 9.26 (s, 1H), 8.25-8.20 (m, 1H), 8.02-7.97 (m, 1H), 7.82-7.74 (m, 1H), 7.49-7.42 (m, 1H), 7.42-7.35 (m, 1H), 7.07-6.97 (m, 2H), 6.04-5.98 (m, 1H), 4.52-4.47 (m, 1H), 4.47-4.41 (m, 2H), 4.45-4.29 (m, 2H), 4.24 (s, 3H), 3.56 (s, 3H), 3.10 (s, 2H), 3.00 (s, 1H), 2.86-2.73 (m, 3H), 2.71-2.58 (m, 2H), 2.41-2.25 (m, OH), 2.22-2.12 (m, 1H), 2.05-1.53 (m, 4H), 1.33 (s, 1H), 1.06-1.00 (m, 6H), 0.76-0.66 (m, 1H), 0.56-0.49 (m, 2H), 0.42-0.33 (m, 1H).

LC-MS (ESI): m/z=854.1 [M+H]⁺.

### Example 240: 3-(7-(4-(((3S, 4S)-1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-methylpiperidin-4-yl)methyl)piperazin-1-yl)-1-methyl-1H-indazol-3-yl)piperidine-2,6-dione (Compound 564a)

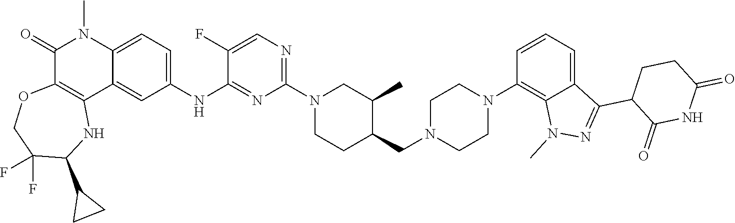 [SMILES: C[C@@H]1CN(c2ncc(F)c(Nc3ccc4c(c3)c3c(c(=O)n4C)OCC(F)(F)[C@H](C4CC4)N3)n2)CC[C@@H]1CN1CCN(c2cccc3c(C4CCC(=O)NC4=O)nn(C)c23)CC1]

### Preparation of benzyl 4-((3S, 4S)-1-(tert-butoxycarbonyl)-3-methylpiperidine-4-carbonyl)piperazine-1-carboxylate

(3S, 4S)-1-(tert-butoxycarbonyl)-3-methylpiperidine-4-carboxylic acid (1500 mg, 1 equiv., 6.165 mmol), benzyl piperazine-1-carboxylate (1.562 g, 1.15 equiv., 7.090 mmol), and HATU (2.813 g, 1.2 equiv., 7.398 mmol) were dissolved in DMF (5 mL). DIEA (2.391 g, 3.22 mL, 3.0 equiv., 18.50 mmol) was then added. The reaction mixture was heated to 50° C. and stirred for 1.5 hours.

The mixture was diluted with ethyl acetate, washed with water, and purified via C₁₈ flash column chromatography (10-100% (A: water+0.1% formic acid; B: MeCN+0.1% formic acid)) to afford benzyl 4-((3S, 4S)-1-(tert-butoxycarbonyl)-3-methylpiperidine-4-carbonyl)piperazine-1-carboxylate (2.74 g, 5.8 mmol). LC-MS (ESI): m/z=446.3 [M+H]⁺.

### Preparation of benzyl 4-(((3S, 4S)-1-(tert-butoxycarbonyl)-3-methylpiperidin-4-yl)methyl)piperazine-1-carboxylate

BH₃-DMS (6.2 g, 7.0 mL, 17 equiv., 81 mmol) was added dropwise to a solution of tert-butyl benzyl 4-((3S, 4S)-1-(tert-butoxycarbonyl)-3-methylpiperidine-4-carbonyl)piperazine-1-carboxylate (2.1648 g, 1.0 equiv., 4.8586 mmol) in THF (20 mL). The reaction mixture was stirred at 50° C. for 1 hour. The mixture was quenched by dropwise addition into methanol (100 mL). The resulting mixture was stirred at 80° C. for 0.5 hour. The mixture was evaporated to dryness to afford benzyl 4-(((3S, 4S)-1-(tert-butoxycarbonyl)-3-methylpiperidin-4-yl)methyl)piperazine-1-carboxylate (1.7526 g, 4.0609 mmol). LC-MS (ESI): m/z=432.3 [M+H]⁺.

### Preparation of tert-butyl (3S, 4S)-3-methyl-4-(piperazin-1-ylmethyl)piperidine-1-carboxylate

Benzyl 4-(((3S, 4S)-1-(tert-butoxycarbonyl)-3-methylpiperidin-4-yl)methyl)piperazine-1-carboxylate (1.75 g, 1 equiv., 4.05 mmol) was dissolved in EtOH (16.0 mL) and degassed with N2.

Palladium hydroxide on carbon (569 mg, 20% Wt, 0.2 equiv., 811 mol) was added. The reaction mixture was charged with excess H₂ and stirred at 50° C. for 1 hour. The mixture was filtered through celite and evaporated to dryness to afford tert-butyl (3S, 4S)-3-methyl-4-(piperazin-1-ylmethyl)piperidine-1-carboxylate (1.22 g, 4.10 mmol). LC-MS (ESI): m/z=298.2 [M+H]⁺.

### Preparation of tert-butyl (3S, 4S)-4-((4-(3-(2,6-bis(benzyloxy)pyridin-3-yl)-1-methyl-1H-indazol-7-yl)piperazin-1-yl)methyl)-3-methylpiperidine-1-carboxylate

Dry N₂(g) was bubbled through a mixture of tert-butyl (3S, 4S)-3-methyl-4-(piperazin-1-ylmethyl)piperidine-1-carboxylate (1000 mg, 1 equiv., 3.362 mmol), 3-(2,6-bis(benzyloxy)pyridin-3-yl)-7-bromo-1-methyl-1H-indazole (3.365 g, 2.0 equiv., 6.724 mmol), and RuPhos-Pd-G3 (246 mg, 0.0875 equiv., 294 mol) in THF (22.0 mL) for 10 minutes. Sodium tert-butoxide (969.3 mg, 5.043 mL, 2.0 molar, 3.0 equiv., 10.09 mmol) was added, and the reaction mixture was heated to 75° C. The resulting mixture was purified via C₁₈ flash column chromatography using a gradient 10-100% (A: water+0.1% formic acid; B: MeCN+0.1% formic acid) to afford tert-butyl (3S, 4S)-4-((4-(3-(2,6-bis(benzyloxy)pyridin-3-yl)-1-methyl-1H-indazol-7-yl)piperazin-1-yl)methyl)-3-methylpiperidine-1-carboxylate (1.798 g, 2.508 mmol). LC-MS (ESI): m/z=717.5[M+H]⁺.

### Preparation of tert-butyl (3S, 4S)-4-((4-(3-(2,6-dioxopiperidin-3-yl)-1-methyl-1H-indazol-7-yl)piperazin-1-yl)methyl)-3-methylpiperidine-1-carboxylate

tert-Butyl (3S, 4S)-4-((4-(3-(2,6-bis(benzyloxy)pyridin-3-yl)-1-methyl-1H-indazol-7-yl)piperazin-1-yl)methyl)-3-methylpiperidine-1-carboxylate (1800 mg, 1 equiv., 2.511 mmol) was dissolved in iPrOH (150 mL)/DMF (15.0 mL) and purged with N₂. The mixture was treated with palladium hydroxide on carbon (352.6 mg, 20% Wt, 0.2 equiv., 502.1 mol) under a hydrogen atmosphere. The reaction mixture was stirred at 50° C. for 18 hours. The mixture was filtered through celite and purified via C₁₈ flash column chromatography (10-100% (A: water+0.1% formic acid; B: MeCN+0.1% formic acid)) to afford tert-butyl (3S, 4S)-4-((4-(3-(2,6-dioxopiperidin-3-yl)-1-methyl-1H-indazol-7-yl)piperazin-1-yl)methyl)-3-methylpiperidine-1-carboxylate (1.15 g, 2.14 mmol). LC-MS (ESI): m/z=539.4 [M+H]⁺.

### Preparation of 3-(1-methyl-7-(4-(((3S, 4S)-3-methylpiperidin-4-yl)methyl)piperazin-1-yl)-1H-indazol-3-yl)piperidine-2,6-dione

tert-Butyl (3S, 4S)-4-((4-(3-(2,6-dioxopiperidin-3-yl)-1-methyl-1H-indazol-7-yl)piperazin-1-yl)methyl)-3-methylpiperidine-1-carboxylate (900 mg, 1 equiv., 1.67 mmol) was dissolved in DCM (2.5 mL) and treated with HCl (487 mg, 3.34 mL, 4.0 molar, 8 equiv., 13.4 mmol). The mixture was stirred at room temperature for 16 hours. The mixture was evaporated to dryness to afford 3-(1-methyl-7-(4-(((3S, 4S)-3-methylpiperidin-4-yl)methyl)piperazin-1-yl)-1H-indazol-3-yl)piperidine-2,6-dione (916.3 mg, 1.730 mmol). LC-MS (ESI): m/z=439.3 [M+H]⁺.

### Preparation of 3-(7-(4-(((3S, 4S)-1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-methylpiperidin-4-yl)methyl)piperazin-1-yl)-1-methyl-1H-indazol-3-yl)piperidine-2,6-dione

A mixture of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (78.3 mg, 1.2 equiv., 173 mol), 3-(1-methyl-7-(4-(((3S, 4S)-3-methylpiperidin-4-yl)methyl)piperazin-1-yl)-1H-indazol-3-yl)piperidine-2,6-dione (76.5 mg, 1.00 equiv., 144 mol), and CsF (110 mg, 5.0 equiv., 722 mol) in DMSO (1.500 mL) was treated with DIEA (131 mg, 176 μL, 7.0 equiv., 1.01 mmol). The mixture was heated to 100° C. and stirred for 1.5 hours. The reaction mixture was directly purified via prep-HPLC to afford 3-(7-(4-(((3S, 4S)-1-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-methylpiperidin-4-yl)methyl)piperazin-1-yl)-1-methyl-1H-indazol-3-yl)piperidine-2,6-dione (12.7 mg, 14.1 mol). ¹H NMR (400 MHz, DMSO) δ 10.87 (s, 1H), 9.22 (s, 1H), 8.21-8.14 (m, 1H), 8.00-7.95 (m, 1H), 7.86-7.78 (m, 1H), 7.49-7.42 (m, 1H), 7.42-7.36 (m, 1H), 7.06-6.98 (m, 2H), 5.99 (s, 1H), 4.51-4.17 (m, 7H), 3.57 (s, 4H), 3.28-2.72 (m, 7H), 2.72-2.55 (m, 1H), 2.40-2.11 (m, 4H), 2.08 (s, 2H), 1.93 (s, 2H), 1.80-1.72 (m, 1H), 1.50-1.27 (m, 1H), 0.81-0.75 (m, 3H), 0.75-0.67 (m, 1H), 0.56-0.49 (m, 2H), 0.41-0.34 (m, 1H). LC-MS (ESI): m/z=854.5 [M+H]⁺.

### Example 241. 3-(6-(1-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperidin-4-yl)-1-methyl-1H-indazol-3-yl)piperidine-2,6-dione (Compound 576a)

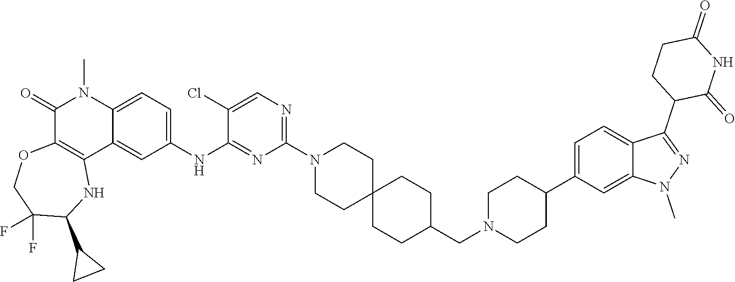 [SMILES: Cn1nc(C2CCC(=O)NC2=O)c2ccc(C3CCN(CC4CCC5(CC4)CCN(c4ncc(Cl)c(Nc6ccc7c(c6)c6c(c(=O)n7C)OCC(F)(F)[C@H](C7CC7)N6)n4)CC5)CC3)cc21]

### Preparation of tert-butyl 9-((4-(3-(2,6-dioxopiperidin-3-yl)-1-methyl-1H-indazol-6-yl)piperidin-1-yl)methyl)-3-azaspiro[5.5]undecane-3-carboxylate

A mixture of tert-butyl 9-formyl-3-azaspiro[5.5]undecane-3-carboxylate (0.927 g, 3.29 mmol), 3-(1-methyl-6-(piperidin-4-yl)-1H-indazol-3-yl)piperidine-2,6-dione, HCl (1.00 g, 2.76 mmol), and potassium acetate (811 mg, 8.27 mmol) was suspended in THF (10.0 mL) and DMF (1.50 mL).

The mixture was stirred at room temperature for 10 min. The white mixture was then treated all at once with sodium triacetoxyborohydride (876 mg, 4.13 mmol), and the reaction mixture was allowed to stir at room temperature for 1 h. The reaction mixture was treated with a saturated aqueous ammonium chloride solution (5 mL) and then extracted with ethyl acetate (3×). The combined organic layers were dried over sodium sulfate, filtered, and concentrated under reduced pressure. The residue was purified by column chromatography to afford tert-butyl 9-((4-(3-(2,6-dioxopiperidin-3-yl)-1-methyl-1H-indazol-6-yl)piperidin-1-yl)methyl)-3-azaspiro[5.5]undecane-3-carboxylate (1.245 g). LC-MS (ESI): m/z=592.4 [M+H]⁺.

### Preparation of 3-(6-(1-((3-azaspiro[5.5]undecan-9-yl)methyl)piperidin-4-yl)-1-methyl-1H-indazol-3-yl)piperidine-2,6-dione tris(trifluoroacetate)

A mixture of tert-butyl 9-((4-(3-(2,6-dioxopiperidin-3-yl)-1-methyl-1H-indazol-6-yl)piperidin-1-yl)methyl)-3-azaspiro[5.5]undecane-3-carboxylate (1.244 g, 2.102 mmol) in DCM (2.00 mL) was treated with a solution of TFA (2.00 mL, 26.0 mmol)/DCM (2.00 mL). The mixture was stirred at room temperature for 1 h. The volatiles were removed under reduced pressure to afford 3-(6-(1-((3-azaspiro[5.5]undecan-9-yl)methyl)piperidin-4-yl)-1-methyl-1H-indazol-3-yl)piperidine-2,6-dione tris(trifluoroacetate) (1.75 g), which was used as is without further purification. LC-MS (ESI): m/z=492.4 [M+H]⁺.

### Preparation of 3-(6-(1-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperidin-4-yl)-1-methyl-1H-indazol-3-yl)piperidine-2,6-dione

A mixture of (S)-10-((5-chloro-2-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (201 mg, 445 mol), 3-(6-(1-((3-azaspiro[5.5]undecan-9-yl)methyl)piperidin-4-yl)-1-methyl-1H-indazol-3-yl)piperidine-2,6-dione tris(trifluoroacetate) (309 mg, 371 mol), and CsF (225 mg, 1.48 mmol) in DMSO (2.00 mL) was warmed to 80° C. for 16 h. The reaction mixture was then cooled to room temperature.

The reaction mixture was filtered with DMSO and purified by prep-HPLC to afford 3-(6-(1-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperidin-4-yl)-1-methyl-1H-indazol-3-yl)piperidine-2,6-dione (154.4 mg) as a white solid. ¹H NMR (400 MHz, DMSO-d₆) δ 10.80 (s, 1H), 8.70 (s, 1H), 8.13 (s, 1H), 7.95 (s, 1H), 7.65 (d, J=9.0 Hz, 1H), 7.55 (s, 1H), 7.37 (d, J=9.3 Hz, 2H), 6.96 (d, J=8.5 Hz, 1H), 6.11 (s, 1H), 4.36 (dd, J=14.0, 6.4 Hz, 1H), 4.31-4.21 (m, 1H), 3.90 (s, 3H), 3.59-3.44 (m, 4H), 3.23 (s, 6H), 3.07-2.81 (m, 4H), 2.62-2.50 (m, 3H), 2.39-2.31 (m, 2H), 2.30-2.14 (m, 2H), 2.09 (dd, J=13.4, 5.6 Hz, 1H), 2.01-1.81 (m, 2H), 1.80-1.56 (m, 2H), 1.55-1.45 (m, 1H), 1.41-1.32 (m, 2H), 1.31-1.18 (m, 3H), 1.18-1.15 (m, 2H), 1.14-0.90 (m, 4H), 0.73-0.55 (m, 1H), 0.45 (t, J=6.0 Hz, 2H), 0.37-0.20 (m, 1H). LC-MS (ESI): m/z=462.4 [M+2H]²+.

### Example 242. 3-(6-(1-((3-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-azaspiro[5.5]undecan-9-yl)methyl)piperidin-4-yl)-1-methyl-1H-indazol-3-yl)piperidine-2,6-dione (Compound 577a)

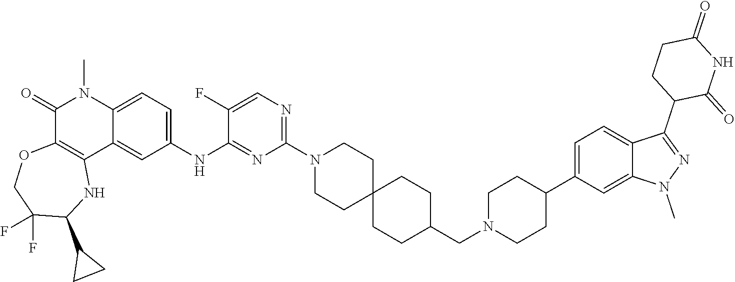 [SMILES: Cn1nc(C2CCC(=O)NC2=O)c2ccc(C3CCN(CC4CCC5(CC4)CCN(c4ncc(F)c(Nc6ccc7c(c6)c6c(c(=O)n7C)OCC(F)(F)[C@H](C7CC7)N6)n4)CC5)CC3)cc21]

### Preparation of 3-(6-(1-((3-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-azaspiro[5.5]undecan-9-yl)methyl)piperidin-4-yl)-1-methyl-1H-indazol-3-yl)piperidine-2,6-dione

A mixture of (S)-10-((2-chloro-5-fluoropyrimidin-4-yl)amino)-2-cyclopropyl-3,3-difluoro-7-methyl-1,2,3,4-tetrahydro-[1,4]oxazepino[2,3-c]quinolin-6(7H)-one (201 mg, 445 mol), 3-(6-(1-((3-azaspiro[5.5]undecan-9-yl)methyl)piperidin-4-yl)-1-methyl-1H-indazol-3-yl)piperidine-2,6-dione tris(trifluoroacetate) (309 mg, 371 mol), and CsF (225 mg, 1.48 mmol) in DMSO (2.00 mL) was warmed to 80° C. and stirred for 16 h, then at 100° C. for 24 h. The reaction mixture was cooled to room temperature, filtered with DMSO, and purified by prep-HPLC to afford 3-(6-(1-((3-(4-(((S)-2-cyclopropyl-3,3-difluoro-7-methyl-6-oxo-1,2,3,4,6,7-hexahydro-[1,4]oxazepino[2,3-c]quinolin-10-yl)amino)-5-fluoropyrimidin-2-yl)-3-azaspiro[5.5]undecan-9-yl)methyl)piperidin-4-yl)-1-methyl-1H-indazol-3-yl)piperidine-2,6-dione (68.5 mg) as a white solid. ¹H NMR (400 MHz, DMSO) δ 10.88 (s, 1H), 9.28 (s, 1H), 8.25 (s, 1H), 8.00 (d, J=3.7 Hz, 1H), 7.76 (d, J=8.9 Hz, 1H), 7.71-7.58 (m, 1H), 7.48-7.38 (m, 2H), 7.04 (d, J=8.7 Hz, 1H), 6.03 (s, 1H), 4.54-4.41 (m, 1H), 4.39-4.27 (m, 1H), 3.98 (s, 3H), 3.67-3.58 (m, 5H), 3.57 (s, 3H), 3.32-3.16 (m, 6H), 3.16-2.84 (m, 4H), 2.73-2.55 (m, 3H), 2.41-2.27 (m, 2H), 2.22-2.11 (m, 1H), 2.09-1.93 (m, 2H), 1.89-1.72 (m, 1H), 1.74-1.66 (m, 1H), 1.66-1.52 (m, 1H), 1.52-1.39 (m, 2H), 1.36-1.27 (m, 3H), 1.27-1.00 (m, 4H), 0.77-0.65 (m, 1H), 0.60-0.46 (m, 2H), 0.43-0.29 (m, 1H). LC-MS (ESI): m/z=454.5 [M+2H]²+.

### Example 243. 3-(6-(1-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperidin-4-yl)-7-fluoro-1-methyl-1H-indazol-3-yl)piperidine-2,6-dione (Compound 581a)

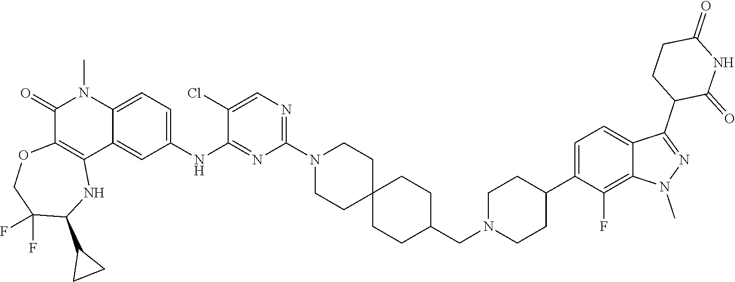 [SMILES: Cn1nc(C2CCC(=O)NC2=O)c2ccc(C3CCN(CC4CCC5(CC4)CCN(c4ncc(Cl)c(Nc6ccc7c(c6)c6c(c(=O)n7C)OCC(F)(F)[C@H](C7CC7)N6)n4)CC5)CC3)c(F)c21]

### Preparation of tert-butyl 4-(3-(2,6-bis(benzyloxy)pyridin-3-yl)-7-fluoro-1-methyl-1H-indazol-6-yl)-3,6-dihydropyridine-1(2H)-carboxylate

To a solution of 3-(2,6-bis(benzyloxy)pyridin-3-yl)-6-bromo-7-fluoro-1-methyl-1H-indazole (1.0 g, 1.93 mmol) in 1,4-dioxane:water (20 mL, 9:1) were added tert-butyl 4-(4,4,5,5-tetramethyl-1,3,2-dioxaborolan-2-yl)-3,6-dihydropyridine-1(2H)-carboxylate (0.71 g, 2.32 mmol) and potassium phosphate (1.6 g, 7.72 mmol). The reaction mixture was purged with argon for 15 min.

Then, PdCl₂(dppf) (0.14 g, 0.19 mmol) was added, and the reaction mixture was stirred at 80° C. for 4 h. After completion, the reaction mixture was filtered through a celite pad. The filtrate was diluted with water (100 mL) and extracted with ethyl acetate (2×100 mL). The organic layer was dried over anhydrous Na₂SO₄, filtered, and concentrated under reduced pressure. The crude was purified by silica gel (100-200) flash column chromatography (0-35% ethyl acetate in petroleum ether) to afford tert-butyl 4-(3-(2,6-bis(benzyloxy)pyridin-3-yl)-7-fluoro-1-methyl-1H-indazol-6-yl)-3,6-dihydropyridine-1(2H)-carboxylate (0.80 g, 60%) as a light green semi solid.

LCMS (ESI): m/z=621.58 (M+H)+

### Preparation of tert-butyl 4-(3-(2,6-dioxopiperidin-3-yl)-7-fluoro-1-methyl-1H-indazol-6-yl)piperidine-1-carboxylate

To a mixture of tert-butyl 4-(3-(2,6-bis(benzyloxy)pyridin-3-yl)-7-fluoro-1-methyl-1H-indazol-6-yl)-3,6-dihydropyridine-1(2H)-carboxylate (0.80 g, 0.17 mmol) in THF:MeOH (1:1; 32 mL) was added 10% Pd/C (0.8 g; w/w). The reaction mixture was kept in a parr shaker under H₂ atmosphere (60 psi) for 6 h. After completion, the reaction mixture was filtered through a celite pad, and the filtrate was concentrated under reduced pressure to afford tert-butyl 4-(3-(2,6-dioxopiperidin-3-yl)-7-fluoro-1-methyl-1H-indazol-6-yl)piperidine-1-carboxylate (0.53 g, 62%) as an off-white solid.

LCMS (ESI): m/z=445.43 (M+H)+

### Preparation of 3-(7-fluoro-1-methyl-6-(piperidin-4-yl)-1H-indazol-3-yl)piperidine-2,6-dione

To a stirred solution of tert-butyl 4-(3-(2,6-dioxopiperidin-3-yl)-7-fluoro-1-methyl-1H-indazol-6-yl)piperidine-1-carboxylate (0.53 g, 5.16 mmol) in DCM (5.3 mL) at 0° C. was added TFA (5.3 mL). The reaction mixture was stirred at room temperature for 2 h. After completion, the reaction mixture was concentrated under reduced pressure. The crude was triturated with diethyl ether and dried under vacuum to afford 3-(7-fluoro-1-methyl-6-(piperidin-4-yl)-1H-indazol-3-yl)piperidine-2,6-dione (0.49 g, 92%) as an off-white solid LCMS (ESI): m/z=345.38 (M+H)+

### Preparation of tert-butyl 9-((4-(3-(2,6-dioxopiperidin-3-yl)-7-fluoro-1-methyl-1H-indazol-6-yl)piperidin-1-yl)methyl)-3-azaspiro[5.5]undecane-3-carboxylate

A mixture of tert-butyl 9-formyl-3-azaspiro[5.5]undecane-3-carboxylate (257 mg, 914 mol), 3-(7-fluoro-1-methyl-6-(piperidin-4-yl)-1H-indazol-3-yl)piperidine-2,6-dione, HCl (290 mg, 761 mol), and potassium acetate (224 mg, 2.28 mmol) was suspended in THF (5.00 mL) and DMF (0.750 mL). The mixture was stirred at room temperature for 15 min. The white mixture was then treated all at once with sodium triacetoxyborohydride (242 mg, 1.14 mmol), and the reaction mixture was allowed to stir at room temperature for 1 h. The reaction mixture was treated with saturated aqueous ammonium chloride solution (5 mL), then extracted with DCM (3×). The combined organics were dried over sodium sulfate, filtered, and concentrated under reduced pressure. The residue was purified by column chromatography to afford tert-butyl 9-((4-(3-(2,6-dioxopiperidin-3-yl)-7-fluoro-1-methyl-1H-indazol-6-yl)piperidin-1-yl)methyl)-3-azaspiro[5.5]undecane-3-carboxylate (378 mg). LC-MS (ESI): m/z=610.4 [M+H]⁺.

### Preparation of 3-(6-(1-((3-azaspiro[5.5]undecan-9-yl)methyl)piperidin-4-yl)-7-fluoro-1-methyl-1H-indazol-3-yl)piperidine-2,6-dione dihydrochloride

A solution of tert-butyl 9-((4-(3-(2,6-dioxopiperidin-3-yl)-7-fluoro-1-methyl-1H-indazol-6-yl)piperidin-1-yl)methyl)-3-azaspiro[5.5]undecane-3-carboxylate (377 mg, 618 mol) in dioxane (2.00 mL) was treated with HCl (4.0 M in dioxane) (4.00 mL, 16.0 mmol). The mixture was stirred at room temperature for 3 h. The volatiles were removed under reduced pressure to afford 3-(6-(1-((3-azaspiro[5.5]undecan-9-yl)methyl)piperidin-4-yl)-7-fluoro-1-methyl-1H-indazol-3-yl)piperidine-2,6-dione dihydrochloride (381 mg), which was used as is without further purification. LC-MS (ESI): m/z=510.3 [M+H]⁺.

### Preparation of 3-(6-(1-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperidin-4-yl)-7-fluoro-1-methyl-1H-indazol-3-yl)piperidine-2,6-dione

A mixture of (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (209 mg, 446 mol), 3-(6-(1-((3-azaspiro[5.5]undecan-9-yl)methyl)piperidin-4-yl)-7-fluoro-1-methyl-1H-indazol-3-yl)piperidine-2,6-dione dihydrochloride (200 mg, 343 mol), and CsF (209 mg, 1.37 mmol) in DMSO (2.00 mL) was treated with DIEA (299 μL, 1.72 mmol). The mixture was warmed to 80° C. and stirred for 24 h, then cooled to room temperature. The reaction mixture was filtered with DMSO and purified by prep-HPLC to afford 3-(6-(1-((3-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-azaspiro[5.5]undecan-9-yl)methyl)piperidin-4-yl)-7-fluoro-1-methyl-1H-indazol-3-yl)piperidine-2,6-dione (62.8 mg) as a white solid. ¹H NMR (400 MHz, DMSO) δ 10.83 (s, 1H), 8.69 (s, 1H), 8.12 (s, 1H), 7.95 (s, 1H), 7.66 (d, J=9.4 Hz, 1H), 7.37 (dd, J=8.9, 3.3 Hz, 2H), 7.08-6.91 (m, 1H), 6.11 (s, 1H), 4.52-4.31 (m, 1H), 4.28 (dd, J=10.4, 5.3 Hz, 1H), 4.04 (s, 3H), 3.59-3.42 (m, 4H), 3.22 (s, 1H), 2.94-2.80 (m, 4H), 2.65-2.50 (m, 2H), 2.35-2.22 (m, 3H), 2.14-2.02 (m, 3H), 1.99-1.86 (m, 2H), 1.78-1.66 (m, 2H), 1.66-1.54 (m, 4H), 1.53-1.46 (m, 2H), 1.45-1.34 (m, 2H), 1.34-1.23 (m, 1H), 1.23-1.14 (m, 4H), 1.07-0.91 (m, 4H), 0.68-0.60 (m, 1H), 0.50-0.39 (m, 2H), 0.28 (s, 1H). LC-MS (ESI): m/z=471.4 [M+2H]²+.

### Example 244: 3-[6-[(2S, 4S)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione (Compound 538c)

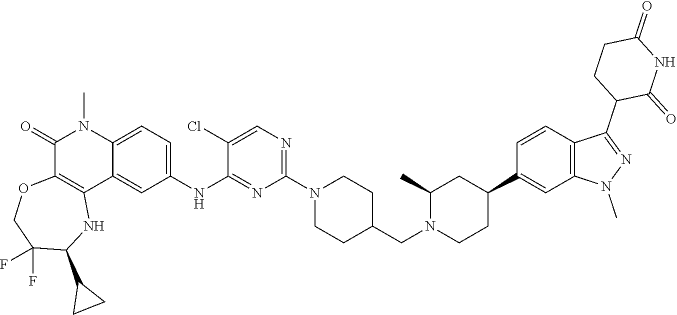 [SMILES: C[C@H]1C[C@@H](c2ccc3c(C4CCC(=O)NC4=O)nn(C)c3c2)CCN1CC1CCN(c2ncc(Cl)c(Nc3ccc4c(c3)c3c(c(=O)n4C)OCC(F)(F)[C@H](C4CC4)N3)n2)CC1]

### Preparation of (2S)-10-amino-2-cyclopropyl-3,3-difluoro-7-methyl-2,4-dihydro-1H-[1,4]oxazepino[2,3-c]quinolin-6-one

A mixture of (2S)-2-cyclopropyl-3,3-difluoro-7-methyl-10-nitro-2,4-dihydro-1H-[1,4]oxazepino[2,3-c]quinolin-6-one (1.5 g, 4.27 mmol, 1 equiv.), Pd/C (4.54 g, 4.27 mmol, 10% purity, 1 equiv.) in EtOH (20 mL) was degassed and purged with H₂ three times. The mixture was stirred at 60° C. for 5 hr under H₂ atmosphere. The reaction mixture was concentrated under reduced pressure to give (2S)-10-amino-2-cyclopropyl-3,3-difluoro-7-methyl-2,4-dihydro-1H-[1,4]oxazepino[2,3-c]quinolin-6-one (1.1 g, crude, yellow solid) which was directly used in the next step without further purification.

LC-MS (ESI): m/z=322.0 [M+H]⁺

### Preparation of (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one

To a solution of (2S)-10-amino-2-cyclopropyl-3,3-difluoro-7-methyl-2,4-dihydro-1H-[1,4]oxazepino[2,3-c]quinolin-6-one (1.1 g, 3.42 mmol, 1 equiv.) and 2,4,5-trichloropyrimidine (753.51 mg, 4.11 mmol, 1.2 equiv.) in NMP (12 mL) was added DIEA (1.77 g, 13.69 mmol, 2.39 mL, 4 equiv.). The mixture was stirred at 140° C. for 1 hr. The reaction mixture was concentrated under reduced pressure to remove solvent. The resulting residue was diluted with water (100 mL) and extracted with ethyl acetate (100 mL×3). The combined organic layers were washed with brine (100 mL×3), dried over Na₂SO₄, filtered, and concentrated under reduced pressure to give a residue. The residue was purified by flash silica gel chromatography (0-50% ethyl acetate/petroleum ether) to give (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (1.4 g, 2.69 mmol) as a yellow solid.

LC-MS (ESI): m/z=468.0 [M+H]⁺.

### Preparation of tert-butyl (2S)-2-methyl-4-(trifluoromethylsulfonyloxy)-3,6-dihydro-2H-pyridine-1-carboxylate

To a solution of tert-butyl (2S)-2-methyl-4-oxo-piperidine-1-carboxylate (10 g, 46.89 mmol, 1 equiv.) in THF (20 mL) was added LiHMDS (1 M, 56.27 mL, 1.2 equiv.) dropwise at −78° C. for 15 min. After addition, the mixture was stirred at this temperature for 30 min, and then 1,1,1-trifluoro-N-phenyl-N-(trifluoromethylsulfonyl)methanesulfonamide (21.78 g, 60.95 mmol, 1.3 equiv.) in THF (20 mL) was added dropwise at −78° C. The resulting mixture was stirred at 25° C. for 12 hr. The reaction mixture was quenched by addition of NH₄Cl (50 mL) at 0° C. The mixture was then diluted with H₂O (300 mL) and extracted with ethyl acetate (300 mL×3). The combined organic layers were washed with brine (300 mL×3), dried over Na₂SO₄, filtered, and concentrated under reduced pressure to give a residue. The residue was purified by column chromatography (SiO₂, petroleum ether/ethyl acetate=100/1 to 5/1) to give tert-butyl (2S)-2-methyl-4-(trifluoromethylsulfonyloxy)-3,6-dihydro-2H-pyridine-1-carboxylate (10 g, 14.48 mmol) as a colorless oil.

LC-MS (ESI): m/z=290.0 [M-tBu+H]⁺.

### Preparation of tert-butyl (2S)-2-methyl-4-(1-methylindazol-6-yl)-3,6-dihydro-2H-pyridine-1-carboxylate

A mixture of tert-butyl (2S)-2-methyl-4-(trifluoromethylsulfonyloxy)-3,6-dihydro-2H-pyridine-1-carboxylate (5 g, 14.48 mmol, 1 equiv.), (1-methylindazol-6-yl)boronic acid (3.06 g, 17.37 mmol, 1.2 equiv.), Na₂CO₃ (4.60 g, 43.44 mmol, 3 equiv.), and Pd(dppf)Cl₂ (1.06 g, 1.45 mmol, 0.1 equiv.) in 1,4-dioxane (35 mL) was degassed and purged with N₂ three times. The mixture was stirred at 95° C. for 12 h under N₂ atmosphere. The reaction mixture was concentrated under reduced pressure to remove 1,4-dioxane. The residue was diluted with water (100 mL) and extracted with ethyl acetate (100 mL×3). The combined organic layers were washed with brine (100 mL×3), dried over Na₂SO₄, filtered, and concentrated under reduced pressure to give a residue. The residue was purified by flash silica gel chromatography (0-10% ethyl acetate/petroleum ether) to give tert-butyl (2S)-2-methyl-4-(1-methylindazol-6-yl)-3,6-dihydro-2H-pyridine-1-carboxylate (1.2 g, 3.48 mmol) as a colorless oil.

LC-MS (ESI): m/z=328.2 [M+H]⁺

### Preparation of tert-butyl (2S)-2-methyl-4-(1-methylindazol-6-yl)piperidine-1-carboxylate

A mixture of tert-butyl (2S)-2-methyl-4-(1-methylindazol-6-yl)-3,6-dihydro-2H-pyridine-1-carboxylate (1.2 g, 3.67 mmol, 1 equiv.) and Pd/C (1.95 g, 1.83 mmol, 10% purity, 0.5 equiv.) in MeOH (3 mL) was degassed and purged with H₂ three times. The mixture was stirred at 25° C. for 5 h under H₂ atmosphere. The reaction mixture was concentrated under reduced pressure to give tert-butyl (2S)-2-methyl-4-(1-methylindazol-6-yl)piperidine-1-carboxylate (1.1 g, crude) which was directly used in the next step without further purification.

LC-MS (ESI): m/z=330.2 [M+H]⁺.

¹H NMR (400 MHz, Chloroform-d) 6=7.84 (s, 1H), 7.57 (d, J=8.4 Hz, 1H), 7.11 (d, J=4.4 Hz, 1H), 6.95 (d, J=8.4 Hz, 1H), 4.69-4.34 (m, 1H), 4.22-4.06 (m, 1H), 3.93 (td, J=6.4, 10.7 Hz, 1H), 3.77 (ddd, J=2.8, 7.6, 14.0 Hz, 1H), 3.41 (s, 1H), 0.22 (ddd, J=6.4, 10.0, 14.0 Hz, 1H), 3.07-2.91 (m, 1H), 2.89-2.78 (m, 1H), 2.24-2.08 (m, 1H), 1.98-1.77 (m, 1H), 1.74-1.53 (m, 2H), 1.43 (d, J=4.0 Hz, 9H), 1.23-1.13 (m, 1H), 1.17 (d, J=6.4 Hz, 1H).

### Preparation of tert-butyl (2S)-4-(3-iodo-1-methyl-indazol-6-yl)-2-methyl-piperidine-1-carboxylate

To a solution of tert-butyl (2S)-2-methyl-4-(1-methylindazol-6-yl)piperidine-1-carboxylate (1.1 g, 3.34 mmol, 1 equiv.) in DMSO (12 mL) was added NIS (1.13 g, 5.01 mmol, 1.5 equiv.). The mixture was stirred at 25° C. for 2 h. The reaction mixture was partitioned between ethyl acetate (200 mL×3) and water (200 mL). The organic phase was separated, washed with brine (100 mL×3), dried over Na₂SO₄, filtered, and concentrated under reduced pressure to give a residue. The residue was purified by flash silica gel chromatography (0-30% ethyl acetate/petroleum ether) to give tert-butyl (2S)-4-(3-iodo-1-methyl-indazol-6-yl)-2-methyl-piperidine-1-carboxylate (850 mg, 1.74 mmol) as a brown oil.

LC-MS (ESI): m/z=456.1 [M+H]⁺

### Preparation of tert-butyl (2S)-4-[3-(2,6-dibenzyloxy-3-pyridyl)-1-methyl-indazol-6-yl]-2-methyl-piperidine-1-carboxylate

A mixture of tert-butyl (2S)-4-(3-iodo-1-methyl-indazol-6-yl)-2-methyl-piperidine-1-carboxylate (800 mg, 1.76 mmol, 1 equiv.), 2,6-dibenzyloxy-3-(4,4,5,5-tetramethyl-1,3,2-dioxaborolan-2-yl)pyridine (733.19 mg, 1.76 mmol, 1 equiv.), Cs₂CO₃ (1.72 g, 5.27 mmol, 3 equiv.), and Pd(dppf)Cl₂ (128.56 mg, 175.70 mol, 0.1 equiv.) in 1,4-dioxane (10 mL) was degassed and purged with N₂ three times. The mixture was stirred at 100° C. for 5 h under N₂ atmosphere. The reaction mixture was partitioned between water (100 mL) and ethyl acetate (100 mL×3). The organic phase was separated, washed with brine (100 mL×3), dried over Na₂SO₄, filtered, and concentrated under reduced pressure to give a residue. The residue was purified by flash silica gel chromatography (0-30% ethyl acetate/petroleum ether) to give tert-butyl (2S)-4-[3-(2,6-dibenzyloxy-3-pyridyl)-1-methyl-indazol-6-yl]-2-methyl-piperidine-1-carboxylate (750 mg, 1.21 mmol) as a white solid.

LC-MS (ESI): m/z=619.7 [M+H]⁺.

### Preparation of tert-butyl (2S, 4S)-4-[3-(2,6-dibenzyloxy-3-pyridyl)-1-methyl-indazol-6-yl]-2-methyl-piperidine-1-carboxylate and tert-butyl (2S, 4R)-4-[3-(2,6-dibenzyloxy-3-pyridyl)-1-methyl-indazol-6-yl]-2-methyl-piperidine-1-carboxylate

The stereoisomers of tert-butyl (2S)-4-[3-(2,6-dibenzyloxy-3-pyridyl)-1-methyl-indazol-6-yl]-2-methyl-piperidine-1-carboxylate (1 g, 1.62 mmol, 1 equiv.) were separated by SFC to give tert-butyl (2S, 4S)-4-[3-(2,6-dibenzyloxy-3-pyridyl)-1-methyl-indazol-6-yl]-2-methyl-piperidine-1-carboxylate (300 mg, 484.84 mol) and tert-butyl (2S, 4R)-4-[3-(2,6-dibenzyloxy-3-pyridyl)-1-methyl-indazol-6-yl]-2-methyl-piperidine-1-carboxylate (170 mg, 274.74 mol) as white solids.

LC-MS (ESI): m/z=619.6 [M+H]⁺.

¹H NMR (400 MHz, DMSO-d6) δ=7.89 (d, J=8.0 Hz, 1H), 7.60 (d, J=8.4 Hz, 1H), 7.49-7.44 (m, 3H), 7.42-7.26 (m, 8H), 6.96 (dd, J=0.8, 8.4 Hz, 1H), 6.62-6.54 (m, 1H), 5.43 (d, J=14.4 Hz, 4H), 4.03 (s, 3H), 3.92-3.83 (m, 1H), 3.72-3.63 (m, 1H), 3.31-3.31 (m, 1H), 3.34-3.26 (m, 2H), 2.91-2.78 (m, 1H), 2.12-2.00 (m, 1H), 1.91-1.83 (m, 1H), 1.79-1.69 (m, 1H), 1.67-1.57 (m, 1H), 1.43 (s, 9H), 1.16 (d, J=6.4 Hz, 3H)

LC-MS (ESI): m/z=619.3 [M+H]⁺.

¹H NMR (400 MHz, DMSO-d6) δ=7.89 (d, J=8.0 Hz, 1H), 7.59 (d, J=8.4 Hz, 1H), 7.51-7.44 (m, 3H), 7.42-7.26 (m, 8H), 6.95 (dd, J=0.8, 8.4 Hz, 1H), 6.58 (d, J=8.0 Hz, 1H), 5.43 (d, J=14.0 Hz, 4H), 4.52-4.34 (m, 1H), 4.04 (s, 3H), 3.98-3.89 (m, 1H), 3.34-3.28 (m, 2H), 3.05 (t, J=12.4 Hz, 1H), 1.86-1.76 (m, 2H), 1.73-1.66 (m, 1H), 1.63-1.54 (m, 1H), 1.43 (s, 9H), 1.21 (s, 3H)

### Preparation of tert-butyl (2S, 4S)-4-[3-(2,6-dioxo-3-piperidyl)-1-methyl-indazol-6-yl]-2-methyl-piperidine-1-carboxylate

To a solution of tert-butyl (2S, 4S)-4-[3-(2,6-dibenzyloxy-3-pyridyl)-1-methyl-indazol-6-yl]-2-methyl-piperidine-1-carboxylate (300 mg, 484.84 mol, 1 equiv.) in CF₃CH₂OH (5 mL) was added Pd/C (515.97 mg, 484.84 mol, 10% purity, 1 equiv.) and Pd(OH)₂ (680.88 mg, 484.84 mol, 10% purity, 1 equiv.). The mixture was stirred at 25° C. for 12 h. The reaction mixture was filtered, and the filtrate was concentrated to give tert-butyl (2S, 4S)-4-[3-(2,6-dioxo-3-piperidyl)-1-methyl-indazol-6-yl]-2-methyl-piperidine-1-carboxylate (150 mg, 340.50 mol) which was used in the next step without further purification.

LC-MS (ESI): m/z=441.1 [M+H]⁺

¹H NMR (400 MHz, DMSO-d6) δ=10.87 (s, 1H), 7.61 (d, J=8.4 Hz, 1H), 7.45 (s, 1H), 7.10-7.00 (m, 1H), 4.32 (dd, J=5.2, 9.6 Hz, 1H), 3.96 (s, 3H), 3.92-3.83 (m, 2H), 3.67 (ddd, J=3.2, 7.2, 13.6 Hz, 1H), 2.89-2.79 (m, 1H), 2.69-2.58 (m, 1H), 2.39-2.28 (m, 2H), 2.16 (qd, J=5.2, 13.2 Hz, 1H), 2.11-2.01 (m, 1H), 1.91-1.83 (m, 1H), 1.80-1.72 (m, 1H), 1.67-1.57 (m, 1H), 1.43 (s, 9H), 1.16 (d, J=6.4 Hz, 3H).

### Preparation of 3-[1-methyl-6-[(2S, 4S)-2-methyl-4-piperidyl]indazol-3-yl]piperidine-2,6-dione

A solution of tert-butyl (2S, 4S)-4-[3-(2,6-dioxo-3-piperidyl)-1-methyl-indazol-6-yl]-2-methyl-piperidine-1-carboxylate (150 mg, 340.50 mol, 1 equiv.) in DCM (2 mL)/HCl/dioxane (1 mL) was stirred at 25° C. for 1 h. The reaction mixture was concentrated under reduced pressure to give 3-[1-methyl-6-[(2S, 4S)-2-methyl-4-piperidyl]indazol-3-yl]piperidine-2,6-dione (100 mg, crude) which was used in the next step without further purification.

LC-MS (ESI): m/z=341.1 [M+H]⁺.

### Preparation of tert-butyl 4-[[(2S, 4S)-4-[3-(2,6-dioxo-3-piperidyl)-1-methyl-indazol-6-yl]-2-methyl-1-piperidyl]methyl]piperidine-1-carboxylate

To a solution of 3-[1-methyl-6-[(2S, 4S)-2-methyl-4-piperidyl]indazol-3-yl]piperidine-2,6-dione (150 mg, 440.63 mol, 1 equiv.) and tert-butyl 4-formylpiperidine-1-carboxylate (140.96 mg, 660.95 mol, 1.5 equiv.) in DCM (2 mL) was added AcOH (26.46 mg, 440.63 mol, 25.22 μL, 1 equiv.) and NaBH(OAc)₃ (140.08 mg, 660.95 mol, 1.5 equiv.). The mixture was stirred at 25° C. for 12 hr. The reaction mixture was concentrated under reduced pressure to give a residue. The residue was purified by flash silica gel chromatography (0-20% ethyl acetate/petroleum ether) to give tert-butyl 4-[[(2S, 4S)-4-[3-(2,6-dioxo-3-piperidyl)-1-methyl-indazol-6-yl]-2-methyl-1-piperidyl]methyl]piperidine-1-carboxylate (180 mg, 334.76 mol) as a white solid.

LC-MS (ESI): m/z=538.4 [M+H]⁺.

¹H NMR (400 MHz, DMSO-d6) δ=10.87 (s, 1H), 7.60 (d, J=8.4 Hz, 1H), 7.42 (s, 1H), 7.02 (d, J=8.4 Hz, 1H), 4.32 (dd, J=5.2, 9.6 Hz, 1H), 4.21-4.05 (m, 1H), 3.97 (s, 4H), 3.93 (s, 1H), 3.16-3.10 (m, 1H), 2.80-2.58 (m, 6H), 2.40-2.28 (m, 2H), 2.21-2.11 (m, 1H), 2.09-1.95 (m, 1H), 1.87-1.49 (m, 7H), 1.39 (s, 9H), 1.10 (d, J=3.6 Hz, 3H), 1.04-0.90 (m, 2H).

### Preparation of 3-[1-methyl-6-[(2S, 4S)-2-methyl-1-(4-piperidylmethyl)-4-piperidyl]indazol-3-yl]piperidine-2,6-dione

A solution of tert-butyl 4-[[(2S, 4S)-4-[3-(2,6-dioxo-3-piperidyl)-1-methyl-indazol-6-yl]-2-methyl-1-piperidyl]methyl]piperidine-1-carboxylate (180.00 mg, 334.76 mol, 1 equiv.) in DCM (2 mL)/TFA (1 mL) was stirred at 25° C. for 1 h. The reaction mixture was concentrated under reduced pressure to remove solvent. The resulting residue was diluted with Na₂CO₃ (aq.) (20 mL) and extracted with ethyl acetate (50 mL). The combined organic layers were filtered and concentrated under reduced pressure to give 3-[1-methyl-6-[(2S, 4S)-2-methyl-1-(4-piperidylmethyl)-4-piperidyl]indazol-3-yl]piperidine-2,6-dione (90 mg, 205.68 mol) which was a used in the next step without further purification.

LC-MS (ESI): m/z=438.2 [M+H]⁺.

### Preparation of 3-[6-[(2S, 4S)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione

A mixture of 3-[1-methyl-6-[(2S, 4S)-2-methyl-1-(4-piperidylmethyl)-4-piperidyl]indazol-3-yl]piperidine-2,6-dione (85 mg, 194.25 mol, 1 equiv.), (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (90.96 mg, 194.25 mol, 1 equiv.), and TEA (78.62 mg, 777.01 mol, 108.15 μL, 4 equiv.) in DMSO (2 mL) was stirred at 100° C. for 2 h. The reaction mixture was partitioned between water (100 mL) and ethyl acetate (300 mL). The organic phase was separated, washed with brine (100 mL×3), dried over Na₂SO₄, filtered, and concentrated under reduced pressure to give a residue. The residue was purified by prep-HPLC to give 3-[6-[(2S, 4S)-1-[[1-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-4-piperidyl]methyl]-2-methyl-4-piperidyl]-1-methyl-indazol-3-yl]piperidine-2,6-dione (56.72 mg, 63.28 mol) as a yellow solid.

LC-MS (ESI): m/z=869.4 [M+H]⁺.

¹H NMR (400 MHz, DMSO-d6) δ=10.87 (s, 1H), 8.77 (s, 1H), 8.22-8.14 (m, 1H), 8.03 (s, 1H), 7.74 (dd, J=1.6, 9.0 Hz, 1H), 7.59 (d, J=8.4 Hz, 1H), 7.44 (d, J=9.2 Hz, 1H), 7.40 (s, 1H), 7.01 (d, J=8.4 Hz, 1H), 6.18 (s, 1H), 4.51-4.36 (m, 3H), 4.32 (dd, J=5.2, 9.6 Hz, 1H), 3.96 (s, 3H), 3.57 (s, 3H), 3.29-3.20 (m, 2H), 3.07 (d, J=11.4 Hz, 1H), 2.83-2.56 (m, 6H), 2.54 (s, 1H), 2.34 (dt, J=4.8, 9.2 Hz, 1H), 2.23 (d, J=6.4 Hz, 1H), 2.20-2.05 (m, 2H), 1.96-1.82 (m, 2H), 1.81-1.67 (m, 4H), 1.61 (d, J=11.2 Hz, 1H), 1.53-1.41 (m, 1H), 1.39-1.26 (m, 1H), 1.08-0.95 (m, 4H), 0.76-0.64 (m, 1H), 0.57-0.46 (m, 2H), 0.40-0.29 (m, 1H)

### Example 245: 3-[6-[[(6S, 7S)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione

### [3-(6-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](Compound 492b)

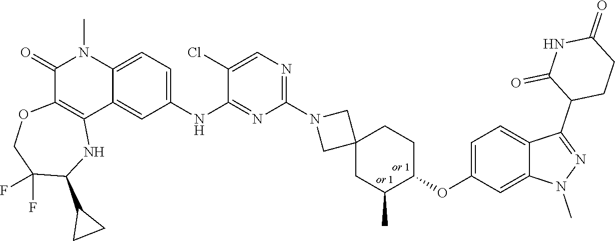 [SMILES: C[C@H]1CC2(CC[C@@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of tert-butyl 6-methyl-7-oxo-2-azaspiro[3.5]nonane-2-carboxylate

To a solution of LiHMDS (55.935 g, 334.28 mL, 1 molar, 2 equiv., 334.28 mmol) in THF (200 mL) under N₂ was added a solution of tert-butyl 7-oxo-2-azaspiro[3.5]nonane-2-carboxylate (4.0 g, 1 equiv., 167.14 mmol) in THF (200 mL) dropwise. The resulting mixture was stirred at −78° C. for 1 h followed by the addition of iodomethane (47.448 g, 20.8 mL, 2 equiv., 334.28 mmol). The mixture was stirred at 25° C. for 12 hours. The reaction mixture was quenched by addition of saturated NH₄Cl solution (200 mL) and extracted with ethyl acetate (70 mL×3). The combined organic layers were washed with brine (70 mL×2), dried over Na₂SO₄, filtered, and concentrated under reduced pressure to give a residue. The residue was purified by column chromatography (petroleum ether/ethyl acetate=10:1) to give tert-butyl 6-methyl-7-oxo-2-azaspiro[3.5]nonane-2-carboxylate (27 g, 0.11 mol) as a white solid.

¹H NMR (400 MHz, DMSO-d6) δ=3.83 (br s, 2H), 3.53 (br s, 2H), 2.48-2.39 (m, 1H), 2.21-2.06 (m, 3H), 1.77 (dt, J=4.4, 13.6 Hz, 1H), 1.52 (t, J=12.8 Hz, 1H), 1.39 (s, 9H), 0.88 (d, J=6.4 Hz, 3H)

### Preparation of tert-butyl 7-hydroxy-6-methyl-2-azaspiro[3.5]nonane-2-carboxylate

To a solution of tert-butyl 6-methyl-7-oxo-2-azaspiro[3.5]nonane-2-carboxylate (26 g, 102.63 mmol, 1 equiv.) in MeOH (300 mL) was added NaBH₄ (11.65 g, 307.89 mmol, 3 equiv.) at 0° C. The mixture was stirred at 0° C. for 1.5 h. The reaction mixture diluted with NH₄Cl (500 mL) and extracted with ethyl acetate (90 mL×3). The combined organic layers were washed with brine (100 mL×2), dried over Na₂SO₄, filtered, and concentrated under reduced pressure to give a residue. The residue was purified by column chromatography (petroleum ether/ethyl acetate=10/1 to 6/1) to afford tert-butyl 7-hydroxy-6-methyl-2-azaspiro[3.5]nonane-2-carboxylate (6.8 g, 21.54 mmol) as a white solid.

LC-MS (ESI): m/z=200.2 [M+H]⁺

¹H NMR: (400 MHz, MeOD-d₄) δ=3.69-3.48 (m, 4H), 3.09-2.94 (m, 1H), 1.93-1.80 (m, 3H), 1.57-1.47 (m, 1H), 1.43 (s, 9H), 1.33-1.18 (m, 3H), 1.01 (br d, J=6.0 Hz, 3H)

### Preparation of tert-butyl (6S, 7R)-7-hydroxy-6-methyl-2-azaspiro[3.5]nonane-2-carboxylate, tert-butyl (6R, 7S)-7-hydroxy-6-methyl-2-azaspiro[3.5]nonane-2-carboxylate, tert-butyl (6S, 7S)-7-hydroxy-6-methyl-2-azaspiro[3.5]nonane-2-carboxylate, and tert-butyl (6R, 7R)-7-hydroxy-6-methyl-2-azaspiro[3.5]nonane-2-carboxylate

The stereoisomers of tert-butyl 7-hydroxy-6-methyl-2-azaspiro[3.5]nonane-2-carboxylate (2.6 g, 10.18 mmol, 1 equiv.) were separated by SFC to afford tert-butyl (6S, 7R)-7-hydroxy-6-methyl-2-azaspiro[3.5]nonane-2-carboxylate, tert-butyl (6R, 7S)-7-hydroxy-6-methyl-2-azaspiro[3.5]nonane-2-carboxylate, tert-butyl (6S, 7S)-7-hydroxy-6-methyl-2-azaspiro[3.5]nonane-2-carboxylate, and tert-butyl (6R, 7R)-7-hydroxy-6-methyl-2-azaspiro[3.5]nonane-2-carboxylate.

LC-MS (ESI): m/z=200.2 [M+H]⁺

LC-MS (ESI): m/z=200.2 [M+H]⁺

LC-MS (ESI): m/z=200.2 [M+H]⁺

LC-MS (ESI): m/z=200.2 [M+H]⁺

¹H NMR: (400 MHz, DMSO-d₆) δ=4.21 (d, J=4.0 Hz, 1H), 3.53-3.39 (m, 4H), 1.75-1.55 (m, 2H), 1.53-1.38 (m, 5H), 1.36 (s, 9H), 0.85 (br d, J=6.0 Hz, 3H). ¹H NMR: (400 MHz, DMSO-d₆) δ=4.21 (d, J=4.0 Hz, 1H), 3.52-3.39 (m, 4H), 1.72-1.55 (m, 2H), 1.52-1.37 (m, 5H), 1.36 (s, 9H), 0.88-0.81 (m, 3H).

¹H NMR: (400 MHz, DMSO-d₆) δ=4.43 (d, J=5.2 Hz, 1H), 3.51 (br s, 2H), 3.42 (br s, 2H), 2.85 (tt, J=4.8, 9.6 Hz, 1H), 1.83-1.63 (m, 3H), 1.36 (s, 9H), 1.22-1.02 (m, 3H), 0.91 (d, J=6.0 Hz, 3H). ¹H NMR: (400 MHz, DMSO-d₆) δ=4.43 (d, J=5.2 Hz, 1H), 3.51 (br s, 2H), 3.42 (br s, 2H), 2.90-2.81 (m, 1H), 1.79-1.65 (m, 3H), 1.36 (s, 9H), 1.21-1.02 (m, 3H), 0.91 (d, J=6.0 Hz, 3H).

### Preparation of tert-butyl (6S, 7S)-7-[3-(2,6-dibenzyloxy-3-pyridyl)-1-methyl-indazol-6-yl]oxy-6-methyl-2-azaspiro[3.5]nonane-2-carboxylate

A mixture of 6-bromo-3-(2,6-dibenzyloxy-3-pyridyl)-1-methyl-indazole (1 g, 2.00 mmol, 1 equiv.), tert-butyl (6S, 7S)-7-hydroxy-6-methyl-2-azaspiro[3.5]nonane-2-carboxylate (1.02 g, 4.00 mmol, 2 equiv.), Ir[dF(CF₃)ppy]₂(dtbpy)(PF₆) (22.42 mg, 19.98 mol, 0.01 equiv.), NiCl₂·dtbbpy (39.77 mg, 99.92 mol, 0.05 equiv.), quinuclidine (22.22 mg, 199.85 mol, 0.1 equiv.), and TMP (564.58 mg, 4.00 mmol, 678.58 μL, 2 equiv.) in MeCN (33 mL) was purged with nitrogen three times. The reaction vial was then sealed with parafilm, placed 2 cm away from one blue LED, and irradiated at 25° C. for 14 h. The reaction mixture was concentrated under reduced pressure to give a residue. The residue was purified by flash silica gel chromatography (30% ethyl acetate/petroleum ether) to afford tert-butyl (6S, 7S)-7-[3-(2,6-dibenzyloxy-3-pyridyl)-1-methyl-indazol-6-yl]oxy-6-methyl-2-azaspiro[3.5]nonane-2-carboxylate (1.4 g, 1.66 mmol) as a white solid.

LC-MS (ESI): m/z=675.5 [M+H]⁺.

¹H NMR (400 MHz, DMSO-d₆) δ=7.90-7.85 (m, 1H), 7.54-7.49 (m, 1H), 7.49-7.44 (m, 2H), 7.42-7.31 (m, 6H), 7.30-7.28 (m, 1H), 6.64-6.53 (m, 2H), 5.43 (d, J=11.2 Hz, 4H), 3.99 (s, 3H), 3.51 (s, 5H), 2.08-1.85 (m, 1H), 1.94-1.84 (m, 2H), 1.78-1.72 (m, 4H), 1.38 (s, 9H), 1.00-0.95 (m, 3H).

### Preparation of tert-butyl (6S, 7S)-7-[3-(2,6-dioxo-3-piperidyl)-1-methyl-indazol-6-yl]oxy-6-methyl-2-azaspiro[3.5]nonane-2-carboxylate

To a solution of tert-butyl (6S, 7S)-7-[3-(2,6-dibenzyloxy-3-pyridyl)-1-methyl-indazol-6-yl]oxy-6-methyl-2-azaspiro[3.5]nonane-2-carboxylate (1.30 g, 1.93 mmol, 1 equiv.) in CF₃CH₂OH (13 mL) was added Pd/C (615.03 mg, 577.93 mol, 10% purity, 0.3 equiv.) and AcOH (11.57 mg, 192.64 mol, 11.03 μL, 0.1 equiv.) under N₂ atmosphere. The suspension was degassed and purged with H₂ three times. The mixture was stirred under H₂ 15 Psi at 25° C. for 16 h. The reaction mixture was filtered and concentrated under reduced pressure to give a residue. The residue was purified by flash silica gel chromatography (60-100% ethyl acetate/petroleum ether) to afford tert-butyl (6S, 7S)-7-[3-(2,6-dioxo-3-piperidyl)-1-methyl-indazol-6-yl]oxy-6-methyl-2-azaspiro[3.5]nonane-2-carboxylate (400 mg, 792.92 mol) as a white solid.

LC-MS (ESI): m/z=497.4 [M+H]⁺

¹H NMR (400 MHz, DMSO-d₆) δ=10.85 (s, 1H), 7.54 (d, J=9.2 Hz, 1H), 7.06 (s, 1H), 6.72 (d, J=8.8 Hz, 1H), 4.35-4.27 (m, 1H), 4.00 (s, 1H), 3.92 (s, 3H), 3.61 (s, 2H), 3.51 (s, 2H), 2.70-2.57 (m, 2H), 2.34-2.28 (m, 1H), 2.19-2.03 (m, 2H), 1.95-1.82 (m, 2H), 1.71-1.56 (m, 2H), 1.38 (s, 9H), 1.32-1.23 (m, 2H), 0.98 (d, J=6.0 Hz, 3H).

### Preparation of 3-[1-methyl-6-[[(6S, 7S)-6-methyl-2-azaspiro[3.5]nonan-7-yl]oxy]indazol-3-yl]piperidine-2,6-dione

To a solution of tert-butyl (6S, 7S)-7-[3-(2,6-dioxo-3-piperidyl)-1-methyl-indazol-6-yl]oxy-6-methyl-2-azaspiro[3.5]nonane-2-carboxylate (400 mg, 805.48 mol, 1 equiv.) in DCM (3 mL) was added TFA (1 mL). The mixture was stirred at 25° C. for 0.5 h. The reaction mixture was concentrated under reduced pressure to give 3-[1-methyl-6-[[(6S, 7S)-6-methyl-2-azaspiro[3.5]nonan-7-yl]oxy]indazol-3-yl]piperidine-2,6-dione (590 mg, crude, TFA) as a colorless gum

LC-MS (ESI): m/z=397.3 [M+H]⁺

### Preparation of 3-[6-[[(6S, 7S)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione [3-(6-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione]

To a solution of (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (100 mg, 213.55 mol, 1 equiv.) in DMSO (1 mL) was added TEA (86.43 mg, 854.18 mol, 118.89 μL, 4 equiv.) and 3-[1-methyl-6-[[(6S, 7S)-6-methyl-2-azaspiro[3.5]nonan-7-yl]oxy]indazol-3-yl]piperidine-2,6-dione (101.60 mg, 256.26 mol, 1.2 equiv.). The mixture was stirred at 100° C. for 2 h. The reaction mixture was filtered to give a residue. The residue was purified by prep-HPLC to afford 3-[6-[[(6S, 7S)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione [3-(6-(((6S*, 7S*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](36.27 mg, 43.03 mol) as a yellow solid.

LC-MS (ESI): m/z=828.2 [M+H]⁺

¹H NMR (400 MHz, DMSO-d₆) δ=10.86 (s, 1H), 8.74 (s, 1H), 8.35-8.18 (m, 1H), 8.03 (s, 1H), 7.94-7.79 (m, 1H), 7.54 (d, J=8.8 Hz, 1H), 7.45 (d, J=9.2 Hz, 1H), 7.07 (s, 1H), 6.72 (d, J=8.8 Hz, 1H), 6.12 (s, 1H), 4.54-4.32 (m, 2H), 4.30-4.27 (m, 1H), 4.07-3.98 (m, 1H), 3.92 (s, 3H), 3.82-3.68 (m, 2H), 3.65 (s, 2H), 3.57 (s, 3H), 2.73-2.56 (m, 2H), 2.36-2.26 (m, 1H), 2.21-2.05 (m, 2H), 2.01-1.85 (m, 2H), 1.77-1.59 (m, 2H), 1.45-1.19 (m, 4H), 0.98 (d, J=5.6 Hz, 3H), 0.78-0.66 (m, 1H), 0.53 (t, J=5.2 Hz, 2H), 0.42-0.30 (m, 1H).

### Example 246: 3-[6-[[(6R, 7R)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione

### [3-(6-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](Compound 492c)

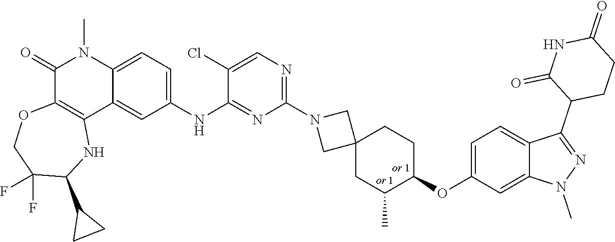 [SMILES: C[C@@H]1CC2(CC[C@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of tert-butyl (6R, 7R)-7-[3-(2,6-dibenzyloxy-3-pyridyl)-1-methyl-indazol-6-yl]oxy-6-methyl-2-azaspiro[3.5]nonane-2-carboxylate

A mixture of 6-bromo-3-(2,6-dibenzyloxy-3-pyridyl)-1-methyl-indazole (1 g, 2.00 mmol, 1 equiv.), tert-butyl (6R, 7R)-7-hydroxy-6-methyl-2-azaspiro[3.5]nonane-2-carboxylate (1.02 g, 4.00 mmol, 2 equiv.), Ir[dF(CF₃)ppy]₂(dtbpy)(PF₆) (22.42 mg, 19.98 mol, 0.01 equiv.), NiCl₂·dtbbpy (39.77 mg, 99.92 mol, 0.05 equiv.), quinuclidine (22.22 mg, 199.85 mol, 0.1 equiv.), and TMP (564.58 mg, 4.00 mmol, 678.58 μL, 2 equiv.) in MeCN (33 mL) was degassed three times. The reaction vial was then sealed with parafilm, placed 2 cm away from one blue LED, and irradiated at 25° C. for 14 h. The reaction mixture was concentrated under reduced pressure to give a residue. The residue was purified by flash silica gel chromatography (20-30% ethyl acetate/petroleum ether) to afford tert-butyl (6R, 7R)-7-[3-(2,6-dibenzyloxy-3-pyridyl)-1-methyl-indazol-6-yl]oxy-6-methyl-2-azaspiro[3.5]nonane-2-carboxylate (1 g, 1.25 mmol) as a yellow solid.

LC-MS (ESI): m/z=675.5 [M+H]⁺.

¹H NMR (400 MHz, DMSO-d₆) δ=7.88 (d, J=8.0 Hz, 1H), 7.54-7.49 (m, 1H), 7.48-7.44 (m, 2H), 7.42-7.32 (m, 6H), 7.29 (d, J=6.8 Hz, 2H), 7.06 (d, J=1.6 Hz, 1H), 6.66-6.54 (m, 2H), 5.43 (d, J=10.8 Hz, 4H), 3.99-3.97 (m, 3H), 3.55-3.38 (m, 5H), 2.12-2.03 (m, 1H), 1.96-1.82 (m, 2H), 1.78-1.62 (m, 4H), 1.38 (s, 9H), 1.00-0.94 (m, 3H)

### Preparation of tert-butyl (6R, 7R)-7-[3-(2,6-dioxo-3-piperidyl)-1-methyl-indazol-6-yl]oxy-6-methyl-2-azaspiro[3.5]nonane-2-carboxylate

To a solution of tert-butyl (6R, 7R)-7-[3-(2,6-dibenzyloxy-3-pyridyl)-1-methyl-indazol-6-yl]oxy-6-methyl-2-azaspiro[3.5]nonane-2-carboxylate (900 mg, 1.33 mmol, 1 equiv.) in CF₃CH₂OH (9 mL) was added Pd/C (425.79 mg, 400.10 mol, 10% purity, 0.3 equiv.) and AcOH (8.01 mg, 133.37 mol, 7.63 μL, 0.1 equiv.) under N₂ atmosphere. The suspension was degassed and purged with H₂ three times. The mixture was stirred under H₂ (15 psi) at 25° C. for 16 h. The reaction mixture was filtered and concentrated under reduced pressure to give a residue. The residue was purified by flash silica gel chromatography (60-100% ethyl acetate/petroleum ether) to afford tert-butyl (6R, 7R)-7-[3-(2,6-dioxo-3-piperidyl)-1-methyl-indazol-6-yl]oxy-6-methyl-2-azaspiro[3.5]nonane-2-carboxylate (320 mg, 637.61 mol) as a white solid.

LC-MS (ESI): m/z=497.4 [M+H]⁺

¹H NMR (400 MHz, DMSO-d₆) δ=10.85 (s, 1H), 7.54 (d, J=8.4 Hz, 1H), 7.06 (s, 1H), 6.72 (d, J=9.6 Hz, 1H), 4.30-4.27 (m, 1H), 4.02-3.96 (m, 1H), 3.92 (s, 3H), 3.69-3.56 (m, 2H), 3.51 (s, 2H), 2.70-2.57 (m, 2H), 2.37-2.23 (m, 1H), 2.20-2.03 (m, 2H), 1.96-1.82 (m, 2H), 1.74-1.51 (m, 2H), 1.35-1.21 (m, 2H), 0.98 (d, J=6.0 Hz, 3H)

### Preparation of 3-[1-methyl-6-[[(6R, 7R)-6-methyl-2-azaspiro[3.5]nonan-7-yl]oxy]indazol-3-yl]piperidine-2,6-dione

To a solution of tert-butyl (6R, 7R)-7-[3-(2,6-dioxo-3-piperidyl)-1-methyl-indazol-6-yl]oxy-6-methyl-2-azaspiro[3.5]nonane-2-carboxylate (320 mg, 644.39 mol, 1 equiv.) in DCM (3 mL) was added TFA (1 mL). The mixture was stirred at 25° C. for 0.5 h. The reaction mixture was concentrated under reduced pressure to give 3-[1-methyl-6-[[(6R, 7R)-6-methyl-2-azaspiro[3.5]nonan-7-yl]oxy]indazol-3-yl]piperidine-2,6-dione (480 mg, crude) as a colorless gum.

LC-MS (ESI): m/z=397.3 [M+H]⁺

### Preparation of 3-[6-[[(6R, 7R)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione [3-(6-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione]

To a solution of (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (100 mg, 213.55 mol, 1 equiv.) in DMSO (1 mL) was added TEA (86.43 mg, 854.18 mol, 118.89 μL, 4 equiv.) and 3-[1-methyl-6-[[(6R, 7R)-6-methyl-2-azaspiro[3.5]nonan-7-yl]oxy]indazol-3-yl]piperidine-2,6-dione (101.60 mg, 256.26 mol, 1.2 equiv.). The mixture was stirred at 100° C. for 2 h. The reaction mixture was filtered to give a residue which was purified by prep-HPLC to afford 3-[6-[[(6R, 7R)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methyl-indazol-3-yl]piperidine-2,6-dione [3-(6-(((6R*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](27.57 mg, 32.03 mol) was obtained as a yellow solid.

LC-MS (ESI): m/z=828.3 [M+H]⁺

¹H NMR (400 MHz, DMSO-d₆) δ=10.85 (s, 1H), 8.73 (s, 1H), 8.23 (s, 1H), 8.03 (s, 1H), 7.94-7.80 (m, 1H), 7.54 (d, J=8.8 Hz, 1H), 7.45 (d, J=8.8 Hz, 1H), 7.07 (s, 1H), 6.72 (d, J=8.8 Hz, 1H), 6.11 (s, 1H), 4.52-4.32 (m, 2H), 4.30-4.27 (m, 1H), 4.07-3.98 (m, 1H), 3.92 (s, 3H), 3.80-3.70 (m, 2H), 3.65 (s, 2H), 3.57 (s, 3H), 2.70-2.56 (m, 2H), 2.35-2.27 (m, 1H), 2.20-2.06 (m, 2H), 2.00-1.86 (m, 2H), 1.76-1.60 (m, 2H), 1.45-1.17 (m, 4H), 0.98 (d, J=6.0 Hz, 3H), 0.79-0.68 (m, 1H), 0.58-0.47 (m, 2H), 0.42-0.30 (m, 1H).

### Example 247: 3-[6-[[(6S, 7R)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methylindazol-3-yl]piperidine-2,6-dione

### [3-(6-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](Compound 492d)

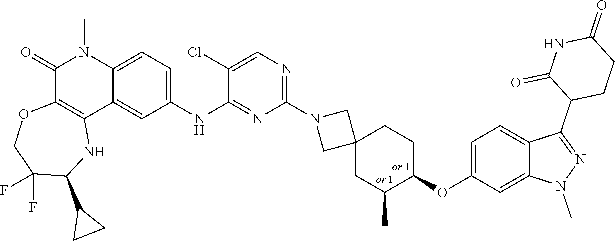 [SMILES: C[C@H]1CC2(CC[C@H]1Oc1ccc3c(C4CCC(=O)NC4=O)nn(C)c3c1)CN(c1ncc(Cl)c(Nc3ccc4c(c3)c3c(c(=O)n4C)OCC(F)(F)[C@H](C4CC4)N3)n1)C2]

### Preparation of tert-butyl (6S, 7R)-7-[3-(2,6-dibenzyloxy-3-pyridyl)-1-methyl-indazol-6-yl]oxy-6-methyl-2-azaspiro[3.5]nonane-2-carboxylate

A mixture of tert-butyl (6S, 7S)-7-hydroxy-6-methyl-2-azaspiro[3.5]nonane-2-carboxylate (0.5 g, 1.96 mmol, 1 equiv.), 3-(2,6-dibenzyloxy-3-pyridyl)-1-methyl-indazol-6-ol (1.03 g, 2.35 mmol, 1.2 equiv.), and 2-(tributylphosphanylidene) acetonitrile (519.85 mg, 2.15 mmol, 1.1 equiv.) in toluene (6 mL) was degassed and purged with N₂ three times. The mixture was stirred at 120° C. for 16 h under N₂ atmosphere. The reaction mixture was concentrated under reduced pressure to remove toluene. The residue was purified by prep-HPLC to afford tert-butyl (6S, 7R)-7-[3-(2,6-dibenzyloxy-3-pyridyl)-1-methyl-indazol-6-yl]oxy-6-methyl-2-azaspiro[3.5]nonane-2-carboxylate (0.52 g, 770.57 mol) as a brown oil.

LC-MS (ESI): m/z=675.4 [M+H]⁺

### Preparation of tert-butyl (6S, 7R)-7-[3-(2,6-dioxo-3-piperidyl)-1-methyl-indazol-6-yl]oxy-6-methyl-2-azaspiro[3.5]nonane-2-carboxylate

A mixture of tert-butyl (6S, 7R)-7-[3-(2,6-dibenzyloxy-3-pyridyl)-1-methyl-indazol-6-yl]oxy-6-methyl-2-azaspiro[3.5]nonane-2-carboxylate (500 mg, 740.93 mol, 1 equiv.), Pd/C (236.55 mg, 222.28 mol, 10% purity, 0.3 equiv.), and AcOH (4.45 mg, 74.09 mol, 4.24 μL, 0.1 equiv.) in CF₃CH₂OH (6 mL) was degassed and purged with H₂ three times. The mixture was stirred at 25° C. for 16 h under H₂ atmosphere. The reaction mixture was filtered, and the filtrate was concentrated to give a residue. The residue was purified by flash silica gel chromatography (0-65% ethyl acetate/petroleum ether) to afford tert-butyl (6S, 7R)-7-[3-(2,6-dioxo-3-piperidyl)-1-methyl-indazol-6-yl]oxy-6-methyl-2-azaspiro[3.5]nonane-2-carboxylate (200 mg, 382.60 mol) as a colorless oil.

LC-MS (ESI): m/z=497.5 [M+H]⁺

### Preparation of 3-[1-methyl-6-[[(6S, 7R)-6-methyl-2-azaspiro[3.5]nonan-7-yl]oxy]indazol-3-yl]piperidine-2,6-dione

A solution of tert-butyl (6S, 7R)-7-[3-(2,6-dioxo-3-piperidyl)-1-methyl-indazol-6-yl]oxy-6-methyl-2-azaspiro[3.5]nonane-2-carboxylate (200 mg, 402.74 mol, 1 equiv.) in DCM (1.5 mL) and TFA (0.5 mL) was stirred at 25° C. for 0.5 h. The reaction mixture was concentrated under reduced pressure to give 3-[1-methyl-6-[[(6S, 7R)-6-methyl-2-azaspiro[3.5]nonan-7-yl]oxy]indazol-3-yl]piperidine-2,6-dione (155 mg, 387.03 mol) as a brown solid.

LC-MS (ESI): m/z=397.3 [M+H]⁺

¹H NMR (400 MHz, DMSO-d₆) δ=10.87 (s, 1H), 8.74-8.52 (m, 2H), 7.55 (d, J=8.8 Hz, 1H), 7.07 (d, J=1.6 Hz, 1H), 6.75 (dd, J=2.0, 8.9 Hz, 1H), 4.48 (s, 1H), 4.29 (dd, J=5.2, 9.6 Hz, 1H), 4.03 (q, J=7.2 Hz, 1H), 3.84-3.70 (m, 2H), 3.70-3.59 (m, 2H), 2.36-2.30 (m, 1H), 2.20-2.09 (m, 1H), 2.03-1.95 (m, 3H), 1.79 (t, J=10.0 Hz, 2H), 1.71-1.58 (m, 2H), 1.52-1.38 (m, 1H), 1.17 (t, J=7.2 Hz, 2H), 0.98 (d, J=6.4 Hz, 3H).

### Preparation of 3-[6-[[(6S, 7R)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methylindazol-3-yl]piperidine-2,6-dione [3-(6-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione]

To a solution of (2S)-2-cyclopropyl-10-[(2,5-dichloropyrimidin-4-yl)amino]-3,3-difluoro-7-methyl-2,4-dihydro-1H-[1,4]oxazepino[2,3-c]quinolin-6-one (100 mg, 213.55 mol, 1 equiv.) and 3-[1-methyl-6-[[(6S, 7R)-6-methyl-2-azaspiro[3.5]nonan-7-yl]oxy]indazol-3-yl]piperidine-2,6-dione (101.60 mg, 256.26 mol, 1.2 equiv.) in DMSO (1.5 mL) was added Et₃N (86.43 mg, 854.18 mol, 118.89 μL, 4 equiv.). The mixture was stirred at 100° C. for 2 h. The reaction mixture was filtered, and the filtrate was concentrated to give a residue which was purified by prep-HPLC to afford 3-[6-[[(6S, 7R)-2-[5-chloro-4-[[(2S)-2-cyclopropyl-3,3-difluoro-7-methyl-6-oxo-2,4-dihydro-1H-[1,4]oxazepino[2,3-c]quinolin-10-yl]amino]pyrimidin-2-yl]-6-methyl-2-azaspiro[3.5]nonan-7-yl]oxy]-1-methylindazol-3-yl]piperidine-2,6-dione [3-(6-(((6S*, 7R*)-2-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-6-methyl-2-azaspiro[3.5]nonan-7-yl)oxy)-1-methyl-1H-indazol-3-yl)piperidine-2,6-dione](34.95 mg, 41.85 μmol) as a yellow solid.

LC-MS (ESI): m/z=828.4 [M+H]⁺.

¹H NMR (400 MHz, DMSO-d₆) δ=10.86 (s, 1H), 8.76 (s, 1H), 8.25 (s, 1H), 8.02 (s, 1H), 7.89-7.77 (m, 1H), 7.56 (d, J=8.8 Hz, 1H), 7.44 (d, J=9.2 Hz, 1H), 7.06 (s, 1H), 6.76 (d, J=8.8 Hz, 1H), 6.27-6.09 (m, 1H), 4.57-4.34 (m, 3H), 4.29 (dd, J=4.8, 9.2 Hz, 1H), 3.91 (s, 3H), 3.79-3.71 (m, 1H), 3.70-3.62 (m, 2H), 3.57 (s, 3H), 2.70-2.59 (m, 2H), 2.33 (s, 3H), 2.20-2.07 (m, 1H), 2.05-1.92 (m, 1H), 1.90-1.77 (m, 1H), 1.75-1.55 (m, 4H), 1.53-1.44 (m, 1H), 1.39-1.25 (m, 1H), 0.98 (d, J=6.4 Hz, 3H), 0.78-0.62 (m, 1H), 0.52 (t, J=5.6 Hz, 2H), 0.42-0.33 (m, 1H).

### Example 248: 3-(6-(((S)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione

### [3-(6-(((S*)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione](Compound 575a)

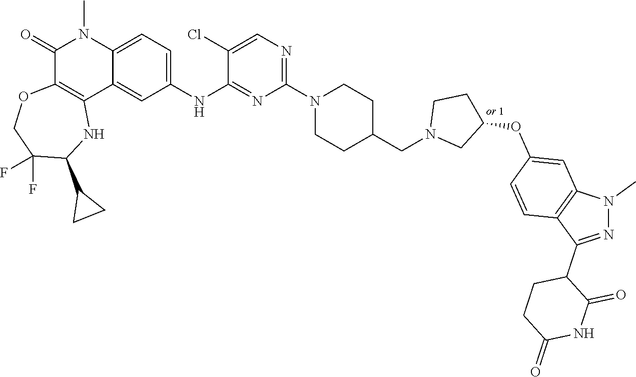 [SMILES: Cn1nc(C2CCC(=O)NC2=O)c2ccc(O[C@H]3CCN(CC4CCN(c5ncc(Cl)c(Nc6ccc7c(c6)c6c(c(=O)n7C)OCC(F)(F)[C@H](C7CC7)N6)n5)CC4)C3)cc21]

### Preparation of 3-(2,6-bis(benzyloxy)pyridin-3-yl)-1-methyl-1H-indazol-6-ol

To a stirred solution of 3-(2,6-bis(benzyloxy)pyridin-3-yl)-6-bromo-1-methyl-1H-indazole (10.0 g, 20.0 mmol) and NaOtBu (5.76 g, 60.00 mmol, 3 equiv.) in THF (100.0 mL) was added water (1.0 mL). The reaction mixture was degassed with nitrogen for 10 minutes. To this solution was added tBuBrettPhos Pd-G3 (0.85 g, 1.0 mmol). The mixture was heated to 80° C. and stirred for 16 h. The reaction mixture was concentrated, diluted with ethyl acetate (500 mL), and washed with water (2×500 mL). The separated organic layer was dried over anhydrous Na₂SO₄, filtered, and concentrated under vacuum to provide a crude product which was purified by column chromatography (40% ethyl acetate in petroleum ether) to afford 3-(2,6-bis(benzyloxy)pyridin-3-yl)-1-methyl-1H-indazol-6-ol (8.70 g) as a brown solid.

LC-MS (ESI): m/z=438.44 [M+H]⁺

### Preparation of tert-butyl 3-((3-(2,6-bis(benzyloxy)pyridin-3-yl)-1-methyl-1H-indazol-6-yl)oxy)pyrrolidine-1-carboxylate

To a stirred solution of 3-(2,6-bis(benzyloxy)pyridin-3-yl)-1-methyl-1H-indazol-6-ol (4.0 g, 9.14 mmol) in toluene (80.0 mL) was added CMBP (4.41 g, 18.29 mmol) and tert-butyl 3-hydroxypyrrolidine-1-carboxylate (5.10 g, 27.43 mmol) at 0° C. The mixture was stirred at 110° C. temperature for 4 h. The reaction mixture was concentrated, diluted with ethyl acetate (2×300 mL), and washed with water (500 mL). The separated organic layer was dried over anhydrous Na₂SO₄, filtered, and concentrated under vacuum to provide a crude product which was purified by column chromatography (50% ethyl acetate in petroleum ether) to afford tert-butyl 3-((3-(2,6-bis(benzyloxy)pyridin-3-yl)-1-methyl-1H-indazol-6-yl)oxy)pyrrolidine-1-carboxylate (5.50 g) as a brown semi solid

LC-MS (ESI): m/z=607.59 [M+H]⁺

### Preparation of 6-(benzyloxy)-3-(1-methyl-6-(pyrrolidin-3-yloxy)-1H-indazol-3-yl)pyridin-2-ol

To a stirred solution of tert-butyl 3-((3-(2,6-bis(benzyloxy)pyridin-3-yl)-1-methyl-1H-indazol-6-yl)oxy)pyrrolidine-1-carboxylate (5.50 g, 9.07 mmol) in DCM (110.0 mL) was added TFA (27.5 mL) at 0° C. The mixture was stirred at room temperature for 2 h. The reaction mixture was concentrated and co-distilled with DCM (3×100 mL) to get a crude product which was triturated with diethyl ether (50 mL) and dried to afford 6-(benzyloxy)-3-(1-methyl-6-(pyrrolidin-3-yloxy)-1H-indazol-3-yl)pyridin-2-ol (5.50 g) as a brown semi-solid.

LC-MS (ESI): m/z=417.34[M+H]⁺

### Preparation of tert-butyl 4-((3-((3-(2-(benzyloxy)-6-hydroxypyridin-3-yl)-1-methyl-1H-indazol-6-yl)oxy)pyrrolidin-1-yl)methyl)piperidine-1-carboxylate

To a stirred solution of 6-(benzyloxy)-3-(1-methyl-6-(pyrrolidin-3-yloxy)-1H-indazol-3-yl)pyridin-2-ol (5.50 g, 13.22 mmol) and tert-butyl 4-formylpiperidine-1-carboxylate (5.63 g, 26.44 mmol) in THF (110.0 mL) was added CH₃COONa (3.25 g, 39.66 mmol) and AcOH (5.50 mL). The mixture was stirred at room temperature for 2 h. To this solution was added sodium triacetoxyborohydride (8.40 g, 39.66 mmol) at 0° C. The resulting solution was stirred at room temperature for 4 h. The reaction mixture was quenched with water (100 mL) and extracted with ethyl acetate (2×200 mL). The combined organic layers were washed with brine, dried over anhydrous Na₂SO₄, filtered, and concentrated under reduced pressure to get a crude product which was purified by column chromatography (10% methanol in DCM) to afford tert-butyl 4-((3-((3-(2-(benzyloxy)-6-hydroxypyridin-3-yl)-1-methyl-1H-indazol-6-yl)oxy)pyrrolidin-1-yl)methyl)piperidine-1-carboxylate (6.0 g) as a pale yellow solid.

LC-MS (ESI): m/z=614.53 [M+H]⁺.

### Preparation of tert-butyl (S)-4-((3-((3-(6-(benzyloxy)-2-hydroxypyridin-3-yl)-1-methyl-1H-indazol-6-yl)oxy)pyrrolidin-1-yl)methyl)piperidine-1-carboxylate and tert-butyl (R)-4-((3-((3-(6-(benzyloxy)-2-hydroxypyridin-3-yl)-1-methyl-1H-indazol-6-yl)oxy)pyrrolidin-1-yl)methyl)piperidine-1-carboxylate

Stereoisomers of tert-butyl 4-((3-((3-(2-(benzyloxy)-6-hydroxypyridin-3-yl)-1-methyl-1H-indazol-6-yl)oxy)pyrrolidin-1-yl)methyl)piperidine-1-carboxylate were separated by chiral HPLC to afford tert-butyl (S)-4-((3-((3-(6-(benzyloxy)-2-hydroxypyridin-3-yl)-1-methyl-1H-indazol-6-yl)oxy)pyrrolidin-1-yl)methyl)piperidine-1-carboxylate and tert-butyl (R)-4-((3-((3-(6-(benzyloxy)-2-hydroxypyridin-3-yl)-1-methyl-1H-indazol-6-yl)oxy)pyrrolidin-1-yl)methyl)piperidine-1-carboxylate as white solids.

### Preparation of tert-butyl 4-(((3S)-3-((3-(2,6-dioxopiperidin-3-yl)-1-methyl-1H-indazol-6-yl)oxy)pyrrolidin-1-yl)methyl)piperidine-1-carboxylate

To a stirred solution of tert-butyl (S)-4-((3-((3-(6-(benzyloxy)-2-hydroxypyridin-3-yl)-1-methyl-1H-indazol-6-yl)oxy)pyrrolidin-1-yl)methyl)piperidine-1-carboxylate (0.55 g, 0.89 mmol) in THF (22.0 mL) was added 20% Pd(OH)₂ (1.10 g). The mixture was stirred under hydrogen pressure (70 psi) at room temperature for 16 h. The reaction mixture was diluted with THF (100 mL), filtered through celite, and washed with THF (200 mL). The filtrate was concentrated and dried to afford tert-butyl 4-(((3S)-3-((3-(2,6-dioxopiperidin-3-yl)-1-methyl-1H-indazol-6-yl)oxy)pyrrolidin-1-yl)methyl)piperidine-1-carboxylate (0.45 g) as an off white solid.

LC-MS (ESI): m/z=526.31 [M+H]⁺

### Preparation of 3-(1-methyl-6-(((S)-1-(piperidin-4-ylmethyl)pyrrolidin-3-yl)oxy)-1H-indazol-3-yl)piperidine-2,6-dione

To a stirred solution of tert-butyl 4-(((3S)-3-((3-(2,6-dioxopiperidin-3-yl)-1-methyl-1H-indazol-6-yl)oxy)pyrrolidin-1-yl)methyl)piperidine-1-carboxylate (0.45 g, 0.86 mmol) in DCM (9.0 mL) was added TFA (2.25 mL) at 0° C. The mixture was stirred at room temperature for 2 h. The reaction mixture was concentrated and co-distilled with DCM (3×50 mL) to get a crude product which was triturated with diethyl ether (15 mL) and dried to afford 3-(1-methyl-6-(((S)-1-(piperidin-4-ylmethyl)pyrrolidin-3-yl)oxy)-1H-indazol-3-yl)piperidine-2,6-dione (0.45 g) as a brown solid.

LC-MS (ESI): m/z=426.44[M+H]⁺

### Preparation of 3-(6-(((S)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione [3-(6-(((S*)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione]

To a stirred solution of 3-(1-methyl-6-(((S)-1-(piperidin-4-ylmethyl)pyrrolidin-3-yl)oxy)-1H-indazol-3-yl)piperidine-2,6-dione (0.30 g, 0.71 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.13 g, 0.28 mmol) in DMSO (6.0 mL) was added N,N-diisopropylethylamine (1.0 mL, 5.64 mmol). The resulting reaction mixture was stirred at 100° C. for 10 h. The reaction mixture was poured into ice cold water (50 mL). The solid precipitate was filtered and dried to get crude product which was purified by prep-HPLC to afford 3-(6-(((S)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione [3-(6-(((S*)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione](0.087 g) as an off-white solid.

LC-MS (ESI): m/z=857.50 [M+H]⁺

¹H NMR (400 MHz, DMSO-d6: δ 10.86 (s, 1H), 8.76 (s, 1H), 8.15 (d, J=2.00 Hz, 1H), 8.02 (s, 1H), 7.74 (dd, J=1.60, 9.00 Hz, 1H), 7.55 (d, J=8.80 Hz, 1H), 7.44 (d, J=9.20 Hz, 1H), 6.96 (d, J=1.60 Hz, 1H), 6.70 (dd, J=2.00, 8.80 Hz, 1H), 6.17 (br, 1H), 4.90 (s, 1H), 4.44-4.28 (m, 5H), 3.92 (s, 3H), 3.56 (s, 3H), 3.23-3.22 (m, 1H), 2.90-2.77 (m, 1H), 2.69-2.67 (m, 2H), 2.65-2.64 (m, 4H), 2.63-2.61 (m, 1H), 2.49-2.38 (m, 1H), 2.35-2.33 (m, 3H), 2.31-2.30 (m, 1H), 1.85-1.69 (m, 4H), 1.40-1.38 (m, 1H), 1.09-0.95 (m, 2H), 0.78-0.65 (m, 1H), 0.51 (t, J=6.00 Hz, 2H), 0.48-0.35 (m, 1H).

### Example 249: 3-(6-(((R)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione

### [3-(6-(((R*)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione](Compound 575b)

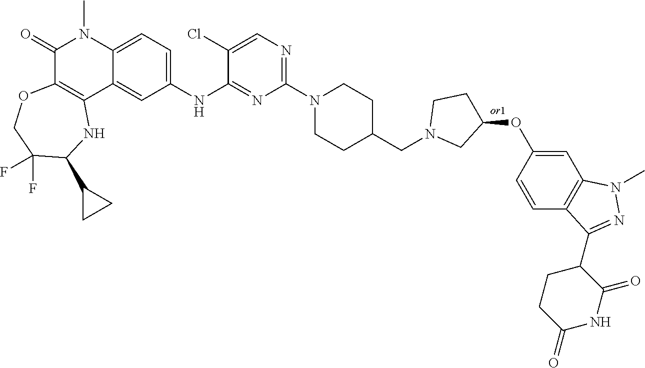 [SMILES: Cn1nc(C2CCC(=O)NC2=O)c2ccc(O[C@@H]3CCN(CC4CCN(c5ncc(Cl)c(Nc6ccc7c(c6)c6c(c(=O)n7C)OCC(F)(F)[C@H](C7CC7)N6)n5)CC4)C3)cc21]

### Preparation of tert-butyl 4-(((3R)-3-((3-(2,6-dioxopiperidin-3-yl)-1-methyl-1H-indazol-6-yl)oxy)pyrrolidin-1-yl)methyl)piperidine-1-carboxylate

To a stirred solution of tert-butyl (R)-4-((3-((3-(6-(benzyloxy)-2-hydroxypyridin-3-yl)-1-methyl-1H-indazol-6-yl)oxy)pyrrolidin-1-yl)methyl)piperidine-1-carboxylate (0.75 g, 1.22 mmol) in THF (30.0 mL) was added 20% Pd(OH)₂ (1.50 g, 200% w/w). The mixture was stirred under hydrogen pressure (70 psi) at room temperature for 16 h. The reaction mixture was diluted with THF (100 mL), filtered through celite, and washed with THF (300 mL). The filtrate was concentrated and dried to afford tert-butyl 4-(((3R)-3-((3-(2,6-dioxopiperidin-3-yl)-1-methyl-1H-indazol-6-yl)oxy)pyrrolidin-1-yl)methyl)piperidine-1-carboxylate (0.60 g) as an off white solid.

LC-MS (ESI): m/z=526.58 [M+H]⁺

### Preparation of 3-(1-methyl-6-(((R)-1-(piperidin-4-ylmethyl)pyrrolidin-3-yl)oxy)-1H-indazol-3-yl)piperidine-2,6-dione

To a stirred solution of tert-butyl 4-(((3R)-3-((3-(2,6-dioxopiperidin-3-yl)-1-methyl-1H-indazol-6-yl)oxy)pyrrolidin-1-yl)methyl)piperidine-1-carboxylate (0.60 g, 1.14 mmol) in DCM (12.0 mL) was added TFA (3.0 mL) at 0° C. The mixture was stirred at room temperature for 2 h. The reaction mixture was concentrated and co-distilled with DCM (3×50 mL) to get a crude product which was triturated with diethyl ether (20 mL) and dried to afford 3-(1-methyl-6-(((R)-1-(piperidin-4-ylmethyl)pyrrolidin-3-yl)oxy)-1H-indazol-3-yl)piperidine-2,6-dione (0.60 g) as a brown solid.

LC-MS (ESI): m/z=426.49[M+H]⁺

### Preparation of 3-(6-(((R)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione [3-(6-(((R*)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione]

To a stirred solution of 3-(1-methyl-6-(((R)-1-(piperidin-4-ylmethyl)pyrrolidin-3-yl)oxy)-1H-indazol-3-yl)piperidine-2,6-dione (0.30 g, 0.71 mmol) and (S)-2-cyclopropyl-10-((2,5-dichloropyrimidin-4-yl)amino)-3,3-difluoro-7-methyl-1,2,3,4-tetrahydro-[1,4]oxazepino [2,3-c]quinolin-6(7H)-one (0.13 g, 0.28 mmol) in DMSO (6.0 mL) was added N,N-diisopropylethylamine (1.0 mL, 5.64 mmol). The resulting reaction mixture was stirred at 100° C. for 10 h. Reaction mixture was poured into ice cold water (50 mL). The solid precipitate was filtered and dried to get a crude product which was purified by prep-HPLC to afford 3-(6-(((R)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione [3-(6-(((R*)-1-((1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)piperidin-4-yl)methyl)pyrrolidin-3-yl)oxy)-1-methyl-1H-indazol-3-yl)piperidine-2,6-dione](0.089 g) as an off-white solid.

LC-MS (ESI): m/z=857.42 [M+H]⁺

¹H NMR (400 MHz, DMSO-d6: δ 10.86 (s, 1H), 8.76 (s, 1H), 8.16-8.15 (m, 1H), 8.02 (s, 1H), 7.74 (dd, J=2.00, 9.00 Hz, 1H), 7.55 (d, J=8.80 Hz, 1H), 7.44 (d, J=9.20 Hz, 1H), 6.95 (d, J=2.00 Hz, 1H), 6.70 (dd, J=2.00, 8.80 Hz, 1H), 6.18 (s, 1H), 5.75 (s, 1H), 4.90 (s, 1H), 4.40-4.31 (m, 4H), 3.92 (s, 3H), 3.61-3.56 (m, 2H), 2.90-2.85 (m, 1H), 2.80-2.75 (m, 2H), 2.70-2.65 (m, 3H), 2.60-2.55 (m, 1H), 2.50-2.45 (m, 2H), 2.40-2.30 (m, 3H), 2.25-2.10 (m, 1H), 1.73-1.70 (m, 4H), 1.49-1.35 (m, 2H), 1.34-1.30 (m, 1H), 1.10-0.95 (m, 2H), 0.75-0.65 (m, 1H), 0.51 (t, J=6.00 Hz, 2H), 0.39-0.25 (m, 1H).

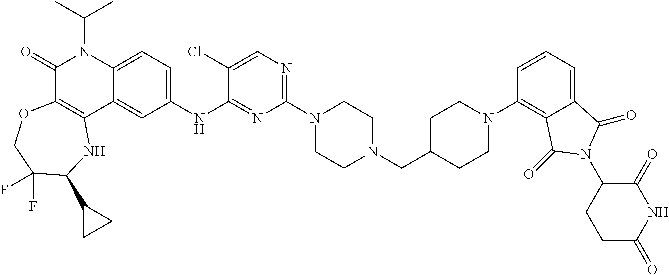 [SMILES: CC(C)n1c(=O)c2c(c3cc(Nc4nc(N5CCN(CC6CCN(c7cccc8c7C(=O)N(C7CCC(=O)NC7=O)C8=O)CC6)CC5)ncc4Cl)ccc31)N[C@@H](C1CC1)C(F)(F)CO2]

### Preparation of tert-butyl 4-((1-(2-(2,6-dioxopiperidin-3-yl)-1,3-dioxoisoindolin-4-yl)piperidin-4-yl)methyl)piperazine-1-carboxylate

To a solution of tert-butyl 4-(piperidin-4-ylmethyl)piperazine-1-carboxylate (1.0 g, 3.52 mmol) in DMSO (20 mL) was added DIPEA (6.14 mL, 35.28 mmol). The reaction mixture was degassed with nitrogen for 1 min. To this solution was added 2-(2,6-dioxopiperidin-3-yl)-4-fluoroisoindoline-1,3-dione (0.48 g, 1.764 mmol). The resulting mixture was heated to 110° C. and stirred for 4 h. The reaction mixture was filtered through celite and concentrated under reduced pressure to get tert-butyl 4-((1-(2-(2,6-dioxopiperidin-3-yl)-1,3-dioxoisoindolin-4-yl)piperidin-4-yl)methyl)piperazine-1-carboxylate (0.850 g) as a white solid.

LC-MS (ESI): m/z=540.66 [M+H]⁺

### Preparation of 2-(2,6-dioxopiperidin-3-yl)-4-(4-(piperazin-1-ylmethyl)piperidin-1-yl)isoindoline-1,3-dione

To a stirred solution of tert-butyl 4-((1-(2-(2,6-dioxopiperidin-3-yl)-1,3-dioxoisoindolin-4-yl)piperidin-4-yl)methyl)piperazine-1-carboxylate (0.85 g, 1.57 mmol) in DCM (8.5 mL) was added 4M HCl in 1,4-dioxane (4.2 mL) dropwise. The reaction mixture was stirred for 16 h at room temperature. The reaction mixture was concentrated under reduced pressure to give a residue which was triturated with diethyl ether to afford 2-(2,6-dioxopiperidin-3-yl)-4-(4-(piperazin-1-ylmethyl)piperidin-1-yl)isoindoline-1,3-dione (0.7 g) as a white solid.

LC-MS (ESI): m/z=440.23 [M+H]⁺

### Preparation of 4-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-2-(2,6-dioxopiperidin-3-yl)isoindoline-1,3-dione

To a stirred solution of 2-(2,6-dioxopiperidin-3-yl)-4-(4-(piperazin-1-ylmethyl)piperidin-1-yl)isoindoline-1,3-dione (0.15 g, 0.341 mmol) in DMSO (3.0 mL) was added DIPEA (0.59 mL, 3.413 mmol). The reaction mixture was degassed with nitrogen for 1 min. To this solution was added (S)-2-cyclopropyl-10-((2,5-dichloropyrimidin-4-yl)amino)-3,3-difluoro-7-isopropyl-1,2,3,4-tetrahydro-[1,4]oxazepino[2,3-c]quinolin-6(7H)-one (0.10 g, 0.205 mmol). The resulting mixture was heated to 130° C. and stirred for 3 h. The reaction mixture was quenched with water (20 mL) and the precipitate was filtered and dried under vacuum to get crude product which was purified by PREP-HPLC to afford 4-(4-((4-(5-chloro-4-(((S)-2-cyclopropyl-3,3-difluoro-7-isopropyl-6-oxo-1,2,3,4,6,7-hexahydro-[1,4]oxazepino[2,3-c]quinolin-10-yl)amino)pyrimidin-2-yl)piperazin-1-yl)methyl)piperidin-1-yl)-2-(2,6-dioxopiperidin-3-yl)isoindoline-1,3-dione (0.029 g) as a white solid.

¹H NMR 400 MHz, DMSO-d6: δ 11.08 (bs, 1H), 8.82 (s, 1H), 8.26 (s, 1H), 8.22 (bs, 1H), 8.04 (s, 1H), 7.71-7.65 (m, 3H), 7.35-7.31 (t, J=4.8 Hz, 2H), 5.07-5.04 (m, 2H), 4.43-4.39 (m, 2H), 3.70-3.61 (bs, 6H), 3.22-2.89 (m, 1H), 2.86-2.64 (t, J=11.9 Hz, 3H), 2.59-2.52 (m, 2H), 2.40-2.38 (m, 4H), 2.21-2.20 (m, 2H), 2.05-2.04 (m, 1H), 1.83-1.80 (d, J=12.0 Hz, 3H), 1.53-1.50 (m, 6H), 1.32-1.29 (d, J=10.6 Hz, 3H), 0.72-0.71 (bs, 1H), 0.54-0.51 (m, 2H), 0.36-0.34 (m, 1H).

LC-MS (ESI): m/z=899.50 [M+H]⁺

### Example 251: 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione

### [3-(6-(4-(((3R*, 4R*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](Compound 578a)

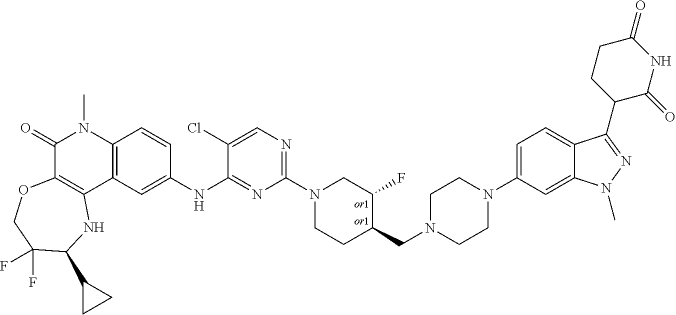 [SMILES: Cn1nc(C2CCC(=O)NC2=O)c2ccc(N3CCN(C[C@H]4CCN(c5ncc(Cl)c(Nc6ccc7c(c6)c6c(c(=O)n7C)OCC(F)(F)[C@H](C7CC7)N6)n5)C[C@@H]4F)CC3)cc21]

### Preparation of trans-tert-butyl-4-(4-(3-(2-(benzyloxy)-6-hydroxypyridin-3-yl)-1-methyl-1H-indazol-6-yl)piperazine-1-carbonyl)-3-fluoropiperidine-1-carboxylate

To a stirred solution of 6-(benzyloxy)-5-(1-methyl-6-(piperazin-1-yl)-1H-indazol-3-yl)pyridin-2-ol (1.40 g, 3.36 mmol) and trans-1-(tert-butoxycarbonyl)-3-fluoropiperidine-4-carboxylic acid (0.83 g, 3.36 mmol) in THF (28.0 mL) were added DIPEA (1.7 mL, 10.08 mmol) and propylphosphonic anhydride (5.34 g, 16.80 mmol) at 0° C. The mixture was stirred at room temperature for 3 h. The reaction mixture was quenched with cold water (40 mL) and extracted with ethyl acetate (3×60 mL). The combined organic layer was dried over anhydrous Na₂SO₄, filtered, and concentrated under reduced pressure to obtain crude product which was purified by flash chromatography (50-100% ethyl acetate in petroleum ether) to afford trans-tert-butyl-4-(4-(3-(2-(benzyloxy)-6-hydroxypyridin-3-yl)-1-methyl-1H-indazol-6-yl)piperazine-1-carbonyl)-3-fluoropiperidine-1-carboxylate (1.01 g) as a pale yellow solid.

LC-MS (ESI): m/z=645.35 [M+H]⁺

### Preparation of trans-tert-butyl-4-((4-(3-(2-(benzyloxy)-6-hydroxypyridin-3-yl)-1-methyl-1H-indazol-6-yl)piperazin-1-yl)methyl)-3-fluoropiperidine-1-carboxylate

To a stirred solution of trans-tert-butyl-4-(4-(3-(2-(benzyloxy)-6-hydroxypyridin-3-yl)-1-methyl-1H-indazol-6-yl)piperazine-1-carbonyl)-3-fluoropiperidine-1-carboxylate (1.01 g, 1.56 mmol) in THF (20 mL) was added BH₃·DMS (neat) (5.0 mL) at 0° C. The mixture was stirred at room temperature for 16 h. The reaction mixture was quenched with MeOH (30 mL), concentrated, and co-distilled with methanol (30.0 mL) to a obtain crude product (as a borane DMS complex). To this residue was added MeOH (150 mL), and the resulting mixture was refluxed for 6 h. The mixture was then cooled to room temperature, concentrated, and dried to afford trans-tert-butyl-4-((4-(3-(2-(benzyloxy)-6-hydroxypyridin-3-yl)-1-methyl-1H-indazol-6-yl)piperazin-1-yl)methyl)-3-fluoropiperidine-1-carboxylate (0.8 g) as an off white solid.

LC-MS (ESI): m/z=631.56 [M+H]⁺

### Preparation of tert-butyl (3S, 4S)-4-((4-(3-(2-(benzyloxy)-6-hydroxypyridin-3-yl)-1-methyl-1H-indazol-6-yl)piperazin-1-yl)methyl)-3-fluoropiperidine-1-carboxylate and tert-butyl (3R, 4R)-4-((4-(3-(2-(benzyloxy)-6-hydroxypyridin-3-yl)-1-methyl-1H-indazol-6-yl)piperazin-1-yl)methyl)-3-fluoropiperidine-1-carboxylate

Stereoisomers of trans-tert-butyl-4-((4-(3-(2-(benzyloxy)-6-hydroxypyridin-3-yl)-1-methyl-1H-indazol-6-yl)piperazin-1-yl)methyl)-3-fluoropiperidine-1-carboxylate (0.8 g) were separated by chiral SFC purification to afford tert-butyl (3R, 4R)-4-((4-(3-(2-(benzyloxy)-6-hydroxypyridin-3-yl)-1-methyl-1H-indazol-6-yl)piperazin-1-yl)methyl)-3-fluoropiperidine-1-carboxylate as an off white solid (0.35 g) and tert-butyl (3S, 4S)-4-((4-(3-(2-(benzyloxy)-6-hydroxypyridin-3-yl)-1-methyl-1H-indazol-6-yl)piperazin-1-yl)methyl)-3-fluoropiperidine-1-carboxylate as an off white solid (0.35 g).

LC-MS (ESI): m/z=631.67 [M+H]⁺

LC-MS (ESI): m/z=631.67 [M+H]⁺

### Preparation of tert-butyl (3R, 4R)-4-((4-(3-(2,6-dioxopiperidin-3-yl)-1-methyl-1H-indazol-6-yl)piperazin-1-yl)methyl)-3-fluoropiperidine-1-carboxylate

To a stirred solution of tert-butyl (3R, 4R)-4-((4-(3-(2-(benzyloxy)-6-hydroxypyridin-3-yl)-1-methyl-1H-indazol-6-yl)piperazin-1-yl)methyl)-3-fluoropiperidine-1-carboxylate (0.35 g, 0.55 mmol.) in THF (35 mL) was added 20% palladium hydroxide (0.35 g, 0.028 mmol.). The mixture was stirred under H₂ (80 psi) pressure for 16 h at room temperature. The reaction mixture was diluted with THF (30 mL), filtered through a celite bed, and washed with THF:DCM (1:1, 20 mL).

The filtrate was concentrated and dried to obtain a crude product which was triturated with diethyl ether and dried to afford tert-butyl (3R, 4R)-4-((4-(3-(2,6-dioxopiperidin-3-yl)-1-methyl-1H-indazol-6-yl)piperazin-1-yl)methyl)-3-fluoropiperidine-1-carboxylate (0.30 g) as a light brown semisolid.

LC-MS (ESI): m/z=543.36 [M+H]⁺

### Preparation of 3-(6-(4-(((3R, 4R)-3-fluoropiperidin-4-yl)methyl)piperazin-1-yl)-1-methyl-1H-indazol-3-yl)piperidine-2,6-dione

To a stirred solution of tert-butyl (3R, 4R)-4-((4-(3-(2,6-dioxopiperidin-3-yl)-1-methyl-1H-indazol-6-yl)piperazin-1-yl)methyl)-3-fluoropiperidine-1-carboxylate (0.30 g, 0.55 mmol) in DCM (9.0 mL) was added TFA (1.5 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL) and dried to afford 3-(6-(4-(((3R, 4R)-3-fluoropiperidin-4-yl)methyl)piperazin-1-yl)-1-methyl-1H-indazol-3-yl)piperidine-2,6-dione (0.20 g) as a pale brown solid.

LC-MS (ESI): m/z=433.28 [M+H]⁺

### Preparation of 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione

### [3-(6-(4-(((3R*, 4R*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione]

To a stirred solution of 3-(6-(4-(((3R, 4R)-3-fluoropiperidin-4-yl)methyl)piperazin-1-yl)-1-methyl-1H-indazol-3-yl)piperidine-2,6-dione (0.20 g, 0.452 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.063 g, 0.136 mmol) in DMSO (2.0 mL) was added DIPEA (2.0 mL). The mixture was stirred at 100° C. for 6 h. The reaction mixture was cooled to room temperature and then treated with ice water. The precipitated solid was filtered and dried to obtain crude a product which was purified by preparative HPLC to afford 3-(6-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione [3-(6-(4-(((3R*, 4R*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](0.043 g) as a pale brown solid.

LC-MS (ESI): m/z=874.49 [M+H]⁺

¹H NMR (400 MHz, DMSO-d6): δ 10.84 (s, 1H), 8.89 (s, 1H), 8.17 (d, J=2.00 Hz, 1H), 8.06 (s, 1H), 7.71 (dd, J=2.00, 9.00 Hz, 1H), 7.47 (q, J=9.20 Hz, 2H), 6.91 (d, J=9.20 Hz, 1H), 6.83 (s, 1H), 6.22 (s, 1H), 4.43-4.23 (m, 5H), 4.20 (brs, 1H), 3.89 (s, 3H), 3.56 (s, 3H), 3.24 (brs, 6H), 3.09 (t, J=12.00 Hz, 1H), 2.70-2.55 (m, 5H), 2.50 (br, 3H), 2.40-2.29 (m, 1H), 2.20-2.16 (m, 1H), 2.13-1.90 (m, 2H), 1.41-1.33 (m, 1H), 1.30-1.15 (m, 1H), 0.80-0.71 (m, 1H), 0.60-0.52 (m, 2H), 0.45-0.35 (m, 1H).

### Example 252: 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl) piperazin-1-yl)-1-methyl-1H-indazol-3-yl)piperidine-2,6-dione

### [3-(6-(4-(((3S*, 4S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](Compound 578b)

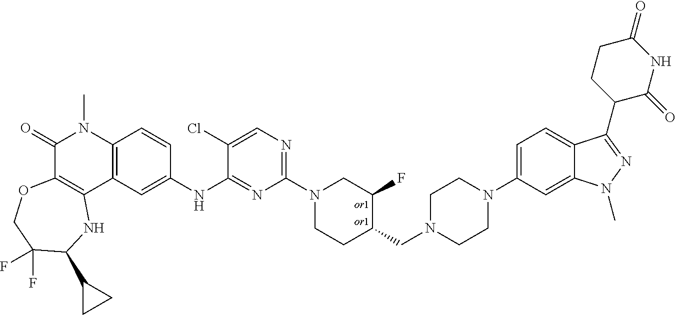 [SMILES: Cn1nc(C2CCC(=O)NC2=O)c2ccc(N3CCN(C[C@@H]4CCN(c5ncc(Cl)c(Nc6ccc7c(c6)c6c(c(=O)n7C)OCC(F)(F)[C@H](C7CC7)N6)n5)C[C@H]4F)CC3)cc21]

### Preparation of tert-butyl (3S, 4S)-4-((4-(3-(2,6-dioxopiperidin-3-yl)-1-methyl-1H-indazol-6-yl)piperazin-1-yl)methyl)-3-fluoropiperidine-1-carboxylate

To a stirred solution of tert-butyl (3S, 4S)-4-((4-(3-(2-(benzyloxy)-6-hydroxypyridin-3-yl)-1-methyl-1H-indazol-6-yl)piperazin-1-yl)methyl)-3-fluoropiperidine-1-carboxylate (0.35 g, 0.55 mmol.) in THF (35 mL) was added 20% palladium hydroxide (0.35 g, 0.028 mmol.). The mixture was stirred under H₂ (80 psi) pressure for 16 h at room temperature. The reaction mixture was diluted with THF (30 mL), filtered through a celite bed, and washed with THF:DCM (1:1, 20 mL).

The filtrate was concentrated and dried to obtain a crude product which was triturated with diethyl ether and dried to afford tert-butyl (3S, 4S)-4-((4-(3-(2,6-dioxopiperidin-3-yl)-1-methyl-1H-indazol-6-yl)piperazin-1-yl)methyl)-3-fluoropiperidine-1-carboxylate (0.30 g) as a light brown semisolid.

LC-MS (ESI): m/z=543.36 [M+H]⁺

### Preparation of 3-(6-(4-(((3S, 4S)-3-fluoropiperidin-4-yl)methyl)piperazin-1-yl)-1-methyl-1H-indazol-3-yl)piperidine-2,6-dione

To a stirred solution of tert-butyl (3S, 4S)-4-((4-(3-(2,6-dioxopiperidin-3-yl)-1-methyl-1H-indazol-6-yl)piperazin-1-yl)methyl)-3-fluoropiperidine-1-carboxylate (0.30 g, 0.55 mmol) in DCM (9.0 mL) was added TFA (1.5 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL) and dried to afford 3-(6-(4-(((3S, 4S)-3-fluoropiperidin-4-yl)methyl)piperazin-1-yl)-1-methyl-1H-indazol-3-yl)piperidine-2,6-dione (0.20 g) as a pale brown solid.

LC-MS (ESI): m/z=443.24 [M+H]⁺

### Preparation of 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl) piperazin-1-yl)-1-methyl-1H-indazol-3-yl)piperidine-2,6-dione

### [3-(6-(4-(((3S*, 4S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione]

To a stirred solution of 3-(6-(4-(((3S, 4S)-3-fluoropiperidin-4-yl)methyl)piperazin-1-yl)-1-methyl-1H-indazol-3-yl)piperidine-2,6-dione (0.20 g, 0.452 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.063 g, 0.136 mmol) in DMSO (2.0 mL) was added DIPEA (2.0 mL). The mixture was stirred at 100° C. for 6 h. The reaction mixture was cooled to room temperature and was treated with ice water. The precipitated solid was filtered and dried to obtain a crude product which was purified by preparative HPLC to afford 3-(6-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl) piperazin-1-yl)-1-methyl-1H-indazol-3-yl)piperidine-2,6-dione [3-(6-(4-(((3S*, 4S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](0.021 g) as a pale brown solid.

LC-MS (ESI): m/z=874.45 [M+H]⁺

¹H NMR (400 MHz, DMSO-d6): δ 10.84 (s, 1H), 8.88 (s, 1H), 8.17 (s, 1H), 8.06 (s, 1H), 7.72 (d, J=7.60 Hz, 1H), 7.47 (q, J=9.20 Hz, 2H), 6.91 (d, J=8.80 Hz, 1H), 6.83 (s, 1H), 6.18 (s, 1H), 4.45-4.23 (m, 5H), 4.06 (br, 1H), 3.89 (s, 3H), 3.56 (s, 3H), 3.30-3.13 (m, 7H), 2.72-2.61 (m, 4H), 2.45 (br, 4H), 2.50-2.22 (m, 1H), 2.22-2.10 (m, 1H), 2.10-1.90 (m, 2H), 1.40-1.30 (m, 1H), 1.25-1.10 (m, 1H), 0.71 (d, J=7.20 Hz, 1H), 0.52 (t, J=5.60 Hz, 2H), 0.34 (d, J=4.80 Hz, 1H).

### Example 253: 3-(7-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione

### [3-(7-(4-(((3S*, 4S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](Compound 579b)

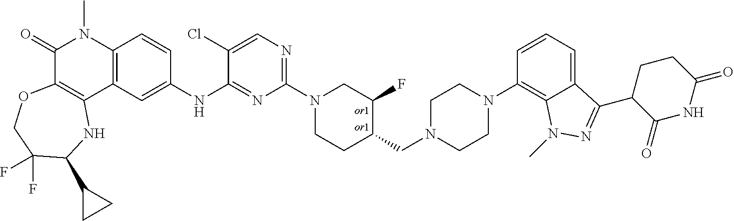 [SMILES: Cn1nc(C2CCC(=O)NC2=O)c2cccc(N3CCN(C[C@@H]4CCN(c5ncc(Cl)c(Nc6ccc7c(c6)c6c(c(=O)n7C)OCC(F)(F)[C@H](C7CC7)N6)n5)C[C@H]4F)CC3)c21]

### Preparation of trans-tert-butyl 4-(4-(3-(2-(benzyloxy)-6-hydroxypyridin-3-yl)-1-methyl-1H-indazol-7-yl)piperazine-1-carbonyl)-3-fluoropiperidine-1-carboxylate

To a stirred solution of 6-(benzyloxy)-5-(1-methyl-7-(piperazin-1-yl)-1H-indazol-3-yl)pyridin-2-ol (1.60 g, 3.85 mmol) and trans-1-(tert-butoxycarbonyl)-3-fluoropiperidine-4-carboxylic acid (0.95 g, 3.85 mmol) in THF (32.0 mL) was added DIPEA (2.0 mL, 11.55 mmol) and stirred for 10 minutes. To this mixture was added propylphosphonic anhydride (6.12 g, 19.25 mmol) at 0° C.

The mixture was stirred at room temperature for 3 h. The reaction mixture was quenched with cold water (40 mL) and extracted with ethyl acetate (3×60 mL). The combined organic layer was dried over anhydrous Na₂SO₄, filtered, and concentrated under reduced pressure to obtain a crude product which was purified by flash chromatography (50-100% ethyl acetate in petroleum ether) to afford trans-tert-butyl 4-(4-(3-(2-(benzyloxy)-6-hydroxypyridin-3-yl)-1-methyl-1H-indazol-7-yl)piperazine-1-carbonyl)-3-fluoropiperidine-1-carboxylate (1.30 g) as a pale-yellow solid.

LC-MS (ESI): m/z=645.35 [M+H]⁺

### Preparation of trans-tert-butyl-4-((4-(3-(2-(benzyloxy)-6-hydroxypyridin-3-yl)-1-methyl-1H-indazol-7-yl)piperazin-1-yl)methyl)-3-fluoropiperidine-1-carboxylate

To a stirred solution of trans-tert-butyl 4-(4-(3-(2-(benzyloxy)-6-hydroxypyridin-3-yl)-1-methyl-1H-indazol-7-yl)piperazine-1-carbonyl)-3-fluoropiperidine-1-carboxylate (1.30 g, 2.01 mmol) in THF (26.0 mL) was added BH₃·DMS (neat) (6.5 mL) at 0° C. The mixture was stirred at room temperature for 16 h. The reaction mixture was quenched with MeOH (30 mL), concentrated, and co-distilled with methanol (30 mL×2) to obtain a crude product (as a Borane DMS complex). To this residue was added MeOH (150 mL) and refluxed at 80° C. for 6 h. The mixture was cooled to room temperature, concentrated, and dried to afford trans-tert-butyl-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (1.20 g) as an off white solid.

LC-MS (ESI): m/z=631.67 [M+H]⁺

### Preparation of tert-butyl (3S, 4S)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate and tert-butyl (3R, 4R)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate

Stereoisomers of trans-tert-butyl-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (1.20 g) were separated by chiral SFC purification to afford tert-butyl (3S, 4S)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate as an off white solid (0.60 g) and tert-butyl (3R, 4R)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate as an off white solid (0.50 g).

### Preparation of tert-butyl (3S, 4S)-4-((4-(3-(2,6-dioxopiperidin-3-yl)-1-methyl-1H-indazol-7-yl)piperazin-1-yl)methyl)-3-fluoropiperidine-1-carboxylate

To a stirred solution of tert-butyl (3S, 4S)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (0.60 g, 0.95 mmol) in THF (60.0 mL) was added 20% palladium hydroxide (0.72 g, 120% w/w). The mixture was stirred under H₂ (80 psi) pressure for 16 h at room temperature. The reaction mixture was diluted with THF (80 mL), filtered through a celite bed, and washed with THF:DCM (1:1, 200 mL). The filtrate was concentrated and dried to obtain a crude product which was triturated with diethyl ether and dried to afford tert-butyl (3S, 4S)-4-((4-(3-(2,6-dioxopiperidin-3-yl)-1-methyl-1H-indazol-7-yl)piperazin-1-yl)methyl)-3-fluoropiperidine-1-carboxylate (0.40 g) as a light brown semi-solid.

LC-MS (ESI): m/z=543.57 [M+H]⁺

### Preparation of 3-(7-(4-(((3S, 4S)-3-fluoropiperidin-4-yl)methyl)piperazin-1-yl)-1-methyl-1H-indazol-3-yl)piperidine-2,6-dione

To a stirred solution of tert-butyl (3S, 4S)-4-((4-(3-(2,6-dioxopiperidin-3-yl)-1-methyl-1H-indazol-7-yl)piperazin-1-yl)methyl)-3-fluoropiperidine-1-carboxylate (0.40 g, 0.74 mmol) in DCM (12.0 mL) was added TFA (2.0 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL) and dried to afford 3-(7-(4-(((3S, 4S)-3-fluoropiperidin-4-yl)methyl)piperazin-1-yl)-1-methyl-1H-indazol-3-yl)piperidine-2,6-dione (0.41 g) as a pale brown solid.

LC-MS (ESI): m/z=443.54[M+H]⁺

### Preparation of 3-(7-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione

### [3-(7-(4-(((3S*, 4S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione]

To a stirred solution of 3-(7-(4-(((3S, 4S)-3-fluoropiperidin-4-yl)methyl)piperazin-1-yl)-1-methyl-1H-indazol-3-yl)piperidine-2,6-dione (0.20 g, 0.45 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.085 g, 0.18 mmol) in DMSO (2.0 mL) was added DIPEA (2.0 mL). The mixture was stirred at 100° C. for 6 h. The reaction mixture was cooled to room temperature and was treated with ice water. The precipitated solid was filtered and dried to obtain crude product which was purified by prep-HPLC to afford 3-(7-(4-(((3S, 4S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione [3-(7-(4-(((3S*, 4S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](0.05 g) as an off white solid.

LC-MS (ESI): m/z=874.49 [M+H]⁺

¹H NMR (400 MHz, DMSO-d6): δ 10.88 (s, 1H), 8.88 (s, 1H), 8.17 (s, 1H), 8.06 (s, 1H), 7.72 (dd, J=2.00, 9.20 Hz, 1H), 7.45 (d, J=9.20 Hz, 1H), 7.40-7.38 (m, 1H), 7.02 (d, J=4.80 Hz, 2H), 6.17 (brs, 1H), 4.42-4.23 (m, 8H), 4.11-4.02 (m, 1H), 3.57 (s, 3H), 3.24-2.71 (m, 9H), 2.67-2.61 (m, 3H), 2.38-2.29 (m, 3H), 2.19-1.90 (m, 4H), 1.38-1.16 (m, 2H), 0.74-0.67 (m, 1H), 0.52 (t, J=6.00 Hz, 2H), 0.35-0.31 (m, 1H).

### Example 254: 3-(7-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione

### [3-(7-(4-(((3R*, 4R*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](Compound 579a)

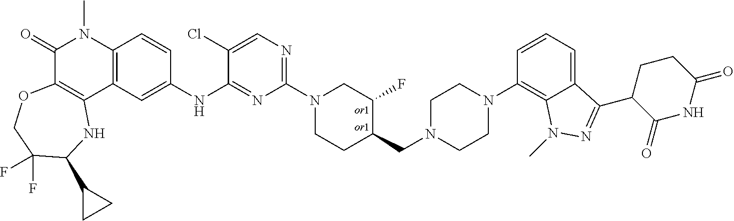 [SMILES: Cn1nc(C2CCC(=O)NC2=O)c2cccc(N3CCN(C[C@H]4CCN(c5ncc(Cl)c(Nc6ccc7c(c6)c6c(c(=O)n7C)OCC(F)(F)[C@H](C7CC7)N6)n5)C[C@@H]4F)CC3)c21]

### Preparation of tert-butyl (3R, 4R)-4-((4-(3-(2,6-dioxopiperidin-3-yl)-1-methyl-1H-indazol-7-yl)piperazin-1-yl)methyl)-3-fluoropiperidine-1-carboxylate

To a stirred solution of tert-butyl (3R, 4R)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (0.50 g, 0.79 mmol) in THF (50.0 mL) was added 20% palladium hydroxide (0.50 g, 100% w/w). The mixture was stirred under H₂ (80 psi) pressure for 16 h at room temperature. The reaction mixture was diluted with THF (50 mL), filtered through a celite bed, and washed with THF:DCM (1:1, 200 mL). The filtrate was concentrated and dried to obtain a crude product which was triturated with diethyl ether and dried to afford tert-butyl (3R, 4R)-4-((4-(3-(2,6-dioxopiperidin-3-yl)-1-methyl-1H-indazol-7-yl)piperazin-1-yl)methyl)-3-fluoropiperidine-1-carboxylate (0.44 g) as a brown semisolid.

LC-MS (ESI): m/z=543.57 [M+H]⁺

### Preparation of 3-(7-(4-(((3R, 4R)-3-fluoropiperidin-4-yl)methyl)piperazin-1-yl)-1-methyl-1H-indazol-3-yl)piperidine-2,6-dione

To a stirred solution of tert-butyl (3R, 4R)-4-((4-(3-(2,6-dioxopiperidin-3-yl)-1-methyl-1H-indazol-7-yl)piperazin-1-yl)methyl)-3-fluoropiperidine-1-carboxylate (0.44 g, 0.81 mmol) in DCM (13.2 mL) was added TFA (2.20 mL) at 0° C. The mixture was stirred at room temperature for 2 h. The reaction mixture was concentrated under reduced pressure and co-distilled with DCM (3×30 mL) to obtain crude product which was triturated with diethyl ether (3×10 mL), and dried to afford 3-(7-(4-(((3R, 4R)-3-fluoropiperidin-4-yl)methyl)piperazin-1-yl)-1-methyl-1H-indazol-3-yl)piperidine-2,6-dione (0.40 g) as an off white solid.

LC-MS (ESI): m/z=443.24 [M+H]⁺

### Preparation of 3-(7-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione

### [3-(7-(4-(((3R*, 4R*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione]

To a stirred solution of 3-(7-(4-(((3R, 4R)-3-fluoropiperidin-4-yl)methyl)piperazin-1-yl)-1-methyl-1H-indazol-3-yl)piperidine-2,6-dione (0.20 g, 0.45 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.085 g, 0.18 mmol) in DMSO (2.0 mL) was added DIPEA (2.0 mL). The mixture was stirred at 100° C. for 6 h. The reaction mixture was cooled to room temperature and was treated with ice water. The precipitated solid was filtered and dried to obtain a crude product which was purified by prep-HPLC to afford 3-(7-(4-(((3R, 4R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione [3-(7-(4-(((3R*, 4R*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3-fluoropiperidin-4-yl)methyl)piperazin-1-yl)-1-methyl-1H-indazol-3-yl)piperidine-2,6-dione](0.035 g) as an off white solid.

LC-MS (ESI): m/z=874.49 [M+H]⁺

¹H NMR (400 MHz, DMSO-d6): δ 10.88 (s, 1H), 8.89 (s, 1H), 8.17 (d, J=2.00 Hz, 1H), 8.06 (s, 1H), 7.71 (dd, J=1.60, 9.00 Hz, 1H), 7.45 (d, J=8.80 Hz, 1H), 7.41-7.37 (m, 1H), 7.03-6.99 (m, 2H), 6.23 (br s, 1H), 4.46-4.24 (m, 8H), 4.17-4.03 (m, 1H), 3.57 (s, 3H), 3.20-2.75 (m, 9H), 2.65-2.50 (m, 4H), 2.50-2.36 (m, 2H), 2.19-2.12 (m, 2H), 2.10-1.91 (m, 2H), 1.45-1.29 (m, 1H), 1.23-1.16 (m, 1H), 0.72-0.67 (m, 1H), 0.53-0.51 (m, 2H), 0.35-0.34 (m, 1H).

### Example 255: 3-(7-(4-(((R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione

### [3-(7-(4-(((R*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione](Compound 582a)

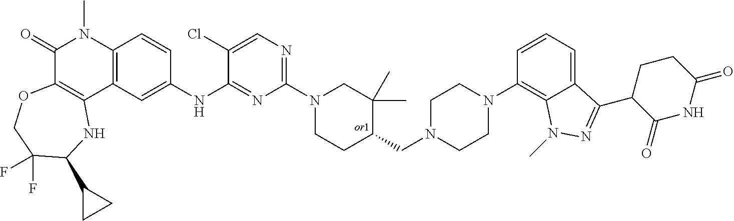 [SMILES: Cn1nc(C2CCC(=O)NC2=O)c2cccc(N3CCN(C[C@@H]4CCN(c5ncc(Cl)c(Nc6ccc7c(c6)c6c(c(=O)n7C)OCC(F)(F)[C@H](C7CC7)N6)n5)CC4(C)C)CC3)c21]

### Preparation of tert-butyl 4-(4-(3-(2-(benzyloxy)-6-hydroxypyridin-3-yl)-1-methyl-1H-indazol-7-yl)piperazine-1-carbonyl)-3,3-dimethylpiperidine-1-carboxylate

To a stirred solution of 6-(benzyloxy)-5-(1-methyl-7-(piperazin-1-yl)-1H-indazol-3-yl)pyridin-2-ol (2.0 g, 4.81 mmol) and 1-(tert-butoxycarbonyl)-3,3-dimethylpiperidine-4-carboxylic acid (1.23 g, 4.81 mmol) in THF (40 mL) was added DIPEA (2.5 mL, 14.45 mmol). The mixture was stirred for 10 minutes. To this mixture was added propylphosphonic anhydride (2.29 g, 7.17 mmol) at 0° C. The resulting mixture was stirred at room temperature for 3 h. The reaction mixture was quenched with cold water (40 mL) and extracted with ethyl acetate (3×60 mL). The combined organic layer was dried over anhydrous Na₂SO₄, filtered, and concentrated under reduced pressure to obtain a crude product which was purified by flash chromatography (50-100% ethyl acetate in petroleum ether) to afford tert-butyl 4-(4-(3-(2-(benzyloxy)-6-hydroxypyridin-3-yl)-1-methyl-1H-indazol-7-yl)piperazine-1-carbonyl)-3,3-dimethylpiperidine-1-carboxylate (0.6 g) as a pale yellow solid.

LC-MS (ESI): m/z=655.35 [M+H]⁺

### Preparation of tert-butyl 4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate

To a stirred solution of tert-butyl 4-(4-(3-(2-(benzyloxy)-6-hydroxypyridin-3-yl)-1-methyl-1H-indazol-7-yl)piperazine-1-carbonyl)-3,3-dimethylpiperidine-1-carboxylate (0.6 g, 0.916 mmol) in THF (12.0 mL) was added BH₃·DMS (neat) (9.0 mL) at 0° C. The mixture was stirred at room temperature for 16 h. The reaction mixture was quenched with MeOH (30 mL), concentrated, and co-distilled with methanol (30 mL×2) to obtain a crude (as a Borane DMS complex). To this residue was added MeOH (150 mL). The resulting mixture was refluxed for 6 h, and then cooled to room temperature, concentrated, and dried to afford tert-butyl 4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate (0.4 g) as an off white solid.

LC-MS (ESI): m/z=641.42 [M+H]⁺

### Preparation of tert-butyl (R)-4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate and tert-butyl (S)-4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate

Stereoisomers of tert-butyl 4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate were separated by SFC purification to afford tert-butyl (R)-4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate and tert-butyl (S)-4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate as white solids.

### Preparation of tert-butyl (4R)-4-((4-(3-(2,6-dioxopiperidin-3-yl)-1-methyl-1H-indazol-7-yl)piperazin-1-yl)methyl)-3,3-dimethylpiperidine-1-carboxylate

To a solution of tert-butyl (R)-4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate (0.14 g, 0.218 mmol) in THF (28 mL) was added DMF (0.5 mL), acetic acid (0.1 mL), and 20% palladium hydroxide on carbon (0.33 g, 300% w/w). The mixture was stirred under hydrogen pressure (80 psi) at room temperature for 16 h. The reaction mixture was diluted with ethyl acetate (50 mL) and filtered through a pad of celite. The filtrate was collected and concentrated to get a crude product which was purified by trituration with diethylether to afford tert-butyl (4R)-4-((4-(3-(2,6-dioxopiperidin-3-yl)-1-methyl-1H-indazol-7-yl)piperazin-1-yl)methyl)-3,3-dimethylpiperidine-1-carboxylate (0.11 g).

LC-MS (ESI): m/z=553.41 [M+H]⁺

### Preparation of 3-(7-(4-(((R)-3,3-dimethylpiperidin-4-yl)methyl)piperazin-1-yl)-1-methyl-1H-indazol-3-yl)piperidine-2,6-dione

To a solution of tert-butyl (4R)-4-((4-(3-(2,6-dioxopiperidin-3-yl)-1-methyl-1H-indazol-7-yl)piperazin-1-yl)methyl)-3,3-dimethylpiperidine-1-carboxylate (0.11 g, 0.199 mmol) in DCM (3.3 mL) was added TFA (0.5 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL) to afford 3-(7-(4-(((R)-3,3-dimethylpiperidin-4-yl)methyl)piperazin-1-yl)-1-methyl-1H-indazol-3-yl)piperidine-2,6-dione (0.13 g) as a pale brown solid.

LC-MS (ESI): m/z=453.60[M+H]⁺

### Preparation of 3-(7-(4-(((R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione

To a stirred solution of 3-(7-(4-(((R)-3,3-dimethylpiperidin-4-yl)methyl)piperazin-1-yl)-1-methyl-1H-indazol-3-yl)piperidine-2,6-dione (0.13 g, 0.28 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.053 g, 0.11 mmol) in DMSO (1.3 mL) was added DIPEA (1.3 mL). The mixture was stirred at 100° C. for 6 h. The reaction mixture was cooled to room temperature and was treated with ice water. The precipitated solid was filtered and dried to obtain crude product which was purified by prep-HPLC to afford 3-(7-(4-(((R)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione (0.031 g) as an off white solid.

LC-MS (ESI): m/z=884.54 [M+H]⁺

¹H NMR (400 MHz, DMSO-d6): δ 10.98 (s, 1H), 8.74 (s, 1H), 8.15 (s, 1H), 8.00 (s, 1H), 7.76 (d, J=7.60 Hz, 1H), 7.45 (d, J=9.20 Hz, 1H), 7.39 (q, J=3.20 Hz, 1H), 7.09-7.01 (m, 2H), 6.19 (brs, 1H), 4.57-4.31 (m, 4H), 4.32 (s, 3H), 4.24 (s, 1H), 3.57 (s, 3H), 3.21-2.71 (m, 7H), 2.65-2.59 (m, 4H), 2.41-2.29 (m, 3H), 2.27-2.10 (m, 2H), 1.79 (d, J=11.20 Hz, 1H), 1.60-1.49 (m, 1H), 1.39-1.20 (m, 3H), 0.92 (brs, 3H), 0.70 (brs, 4H), 0.52 (t, J=6.0 Hz, 2H), 0.40-0.39 (m, 1H).

### Example 256: 3-(7-(4-(((S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione

### [3-(7-(4-(((S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione](Compound 582b)

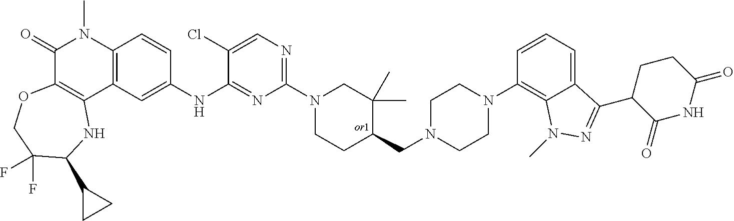 [SMILES: Cn1nc(C2CCC(=O)NC2=O)c2cccc(N3CCN(C[C@H]4CCN(c5ncc(Cl)c(Nc6ccc7c(c6)c6c(c(=O)n7C)OCC(F)(F)[C@H](C7CC7)N6)n5)CC4(C)C)CC3)c21]

### Preparation of tert-butyl (4S)-4-((4-(3-(2,6-dioxopiperidin-3-yl)-1-methyl-1H-indazol-7-yl)piperazin-1-yl)methyl)-3,3-dimethylpiperidine-1-carboxylate

A stirred solution of tert-butyl (S)-4-((4-(3-(2-(benzyloxy)-6-hydroxypyridin-3-yl)-1-methyl-1H-indazol-7-yl)piperazin-1-yl)methyl)-3,3-dimethylpiperidine-1-carboxylate (0.1 g, 0.156 mmol) in THF (20 mL) was added DMF (0.5 mL), acetic acid (0.1 mL), and 20% palladium hydroxide on carbon (0.30 g, 300% w/w). The mixture was stirred under hydrogen pressure (80 psi) at room temperature for 16 h. The reaction mixture was diluted with ethyl acetate (50 mL) and filtered through a pad of celite. The filtrate was collected and concentrated to get a crude product which was purified by trituration with diethylether to afford tert-butyl (4S)-4-((4-(3-(2,6-dioxopiperidin-3-yl)-1-methyl-1H-indazol-7-yl)piperazin-1-yl)methyl)-3,3-dimethylpiperidine-1-carboxylate (0.10 g).

LC-MS (ESI): m/z=553.41 [M+H]⁺

### Preparation of 3-(7-(4-(((S)-3,3-dimethylpiperidin-4-yl)methyl)piperazin-1-yl)-1-methyl-1H-indazol-3-yl)piperidine-2,6-dione

To a stirred solution of tert-butyl (4S)-4-((4-(3-(2,6-dioxopiperidin-3-yl)-1-methyl-1H-indazol-7-yl)piperazin-1-yl)methyl)-3,3-dimethylpiperidine-1-carboxylate (0.10 g, 0.18 mmol) in DCM (3.0 mL) was added TFA (0.5 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL) and dried to afford 3-(7-(4-(((S)-3,3-dimethylpiperidin-4-yl)methyl)piperazin-1-yl)-1-methyl-1H-indazol-3-yl)piperidine-2,6-dione (0.14 g) as a pale brown solid.

LC-MS (ESI): m/z=453.29 [M+H]⁺

### Preparation of 3-(7-(4-(((S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione [3-(7-(4-(((S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione]

To a stirred solution of 3-(7-(4-(((S)-3,3-dimethylpiperidin-4-yl)methyl)piperazin-1-yl)-1-methyl-1H-indazol-3-yl)piperidine-2,6-dione (0.14 g, 0.30 mmol) and (S)-2-cyclopropyl-10-((2,5-dichloropyrimidin-4-yl)amino)-3,3-difluoro-7-methyl-1,2,3,4-tetrahydro-[1,4]oxazepino[2,3-c]quinolin-6(7H)-one (0.057 g, 0.12 mmol) in DMSO (1.4 mL) was added DIPEA (1.4 mL). The mixture was stirred at 100° C. for 6 h. The reaction mixture was cooled to room temperature and was treated with ice water. The precipitated solid was filtered and dried to obtain a crude product which was purified by prep-HPLC to afford 3-(7-(4-(((S)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione [3-(7-(4-(((S*)-1-(5-chloro-4-(((S)-2-cyclopropyl-3,3-difluoro-7-methyl-6-oxo-1,2,3,4,6,7-hexahydro-[1,4]oxazepino[2,3-c]quinolin-10-yl)amino)pyrimidin-2-yl)-3,3-dimethylpiperidin-4-yl)methyl)piperazin-1-yl)-1-methyl-1H-indazol-3-yl)piperidine-2,6-dione](0.03 g) as an off white solid.

LC-MS (ESI): m/z=884.54 [M+H]⁺

¹H NMR (400 MHz, DMSO-d6): δ 10.87 (s, 1H), 8.76 (s, 1H), 8.34 (s, 1H), 8.00 (s, 1H), 7.75 (dd, J=1.60, 9.00 Hz, 1H), 7.45 (d, J=9.20 Hz, 1H), 7.40-7.38 (m, 1H), 7.09-7.02 (m, 2H), 6.17 (s, 1H), 4.57-4.30 (m, 4H), 4.32 (s, 3H), 4.23 (s, 1H), 3.57 (s, 3H), 3.14-2.72 (m, 8H), 2.67-2.58 (m, 3H), 2.38-2.32 (m, 2H), 2.18-2.10 (m, 3H), 1.81-1.73 (m, 1H), 1.54-1.49 (m, 1H), 1.40-1.12 (m, 3H), 0.94 (s, 3H), 0.71 (s, 4H), 0.52 (t, J=6.00 Hz, 2H), 0.35-0.36 (m, 1H).

Additional compounds of this disclosure are synthesized using methods similar to that described in the preceding examples.

The following examples were synthesized using methods similar to those described in the preceding examples:

| Example | Compound | LCMS | |
|---|---|---|---|
| No. | No. | (ESI) | ¹H NMR |
| 181 | 496a | m/z = | ¹H NMR (400 MHz, DMSO-d6): δ 10.87 (s, 1H), 8.08- |
| | | 869 | 8.04 (m, 1H), 7.99 (s, 1H), 7.92 (s, 1H), 7.53-7.43 (m, |
| | | M + H]⁺ | 2H), 7.41-7.35 (m, 1H), 7.04-6.94 (m, 2H), 6.41 (s, 1H), |
| | | | 6.09 (s, 1H), 4.49-4.28 (m, 3H), 4.26-4.20 (m, 2H), 4.04- |
| | | | 3.91 (m, 2H), 3.58 (s, 3H), 3.24-2.74 (m, 1H), 2.74- |
| | | | 2.56 (m, 3H), 2.45-2.21 (m, 1H), 2.21-1.74 (m, 1H), |
| | | | 1.74-1.38 (m, 7H), 1.38-1.18 (m, 3H), 1.10-0.93 (m, |
| | | | 2H), 0.71 (s, 1H), 0.50 (s, 2H), 0.31 (s, 1H), 0.10-0.05 |
| | | | (m, 2H). |
| 182 | 497a | m/z = | ¹H NMR (400 MHz, DMSO-d6): δ (ppm) = 10.80 (s, 1H), |
| | | 887.7 | 8.76 (s, 1H), 8.14 (d, J = 1.1 Hz, 1H), 7.99 (s, 1H), 7.65 (br |
| | | [M + H]⁺ | d, J = 9.0 Hz, 1H), 7.38 (d, J = 9.1 Hz, 1H), 7.34-7.27 |
| | | | (m, 1H), 7.03-6.89 (m, 2H), 6.14 (br s, 1H), 4.44-4.30 |
| | | | (m, 2H), 4.26 (br dd, J = 5.1, 9.6 Hz, 2H), 4.17 (s, 4H), |
| | | | 3.50 (s, 3H), 3.06 (br s, 4H), 2.88-2.70 (m, 1H), 2.63- |
| | | | 2.47 (m, 4H), 2.33-2.18 (m, 2H), 2.09 (br dd, J = 5.6, |
| | | | 13.3 Hz, 1H), 1.99-1.88 (m, 1H), 1.79-1.41 (m, 3H), |
| | | | 1.27 (br dd, J = 4.6, 8.9 Hz, 1H), 0.94 (br s, 2H), 0.71- |
| | | | 0.58 (m, 1H), 0.45 (br t, J = 5.6 Hz, 2H), 0.35-0.22 (m, |
| | | | 1H). |
| 183 | 498a | m/z = | ¹H NMR (400 MHz, DMSO-d6): δ 10.87 (s, 1H), 8.98- |
| | | 882.49 | 8.86 (m, 1H), 8.11-8.03 (m, 2H), 7.65 (s, 1H), 7.43-7.38 |
| | | [M + H]⁺ | (m, 2H), 6.95-6.87 (m, 2H), 6.23 (s, 1H), 4.31-4.24 (m, |
| | | | 6H), 3.57 (s, 4H), 3.23-3.16 (m, 8H), 2.85-260 (m, 5H), |
| | | | 2.40-2.30 (m,1H), 2.20-2.10 (m, 1H), 2.15-2.14 (m, 1H), |
| | | | 1.99-1.77 (m, 3H), 1.45-1.22 (m, 5H), 0.90-0.80 (m, 2H), |
| | | | 0.72-0.66 (m, 1H), 0.52-0.40 (m, 2H), 0.4-0.3 (m, 1H). |
| 184 | 500a | m/z = | ¹H NMR (400 MHz, DMSO-d6): δ 10.90 (s, 1H), 8.76 (s |
| | | 885.47 | 1H), 8.19 (d, J = 1.6 Hz, 1H), 8.03 (s, 1H), 7.76 (dd, J = |
| | | [M + H]⁺ | 8.8, 1.6 Hz, 1H), 7.45 (d, J = 8.0 Hz, 1H), 7.37-7.32 (m, |
| | | | 2H), 7.03-6.96 (m, 2H), 6.18 (bs, 1H), 4.57-4.31 (m, 5H), |
| | | | 4.24 (s, 3H), 3.90 (s, 1H), 3.57 (s, 3H), 2.71-2.58 (m, 6H), |
| | | | 2.36-2.35 (m, 1H), 2.20-2.17 (m, 1H), 1.71-1.62 (m, 7H), |
| | | | 1.23-1.21 (m, 5H), 1.11-1.07 (m, 1H), 0.95 (s, 3H), 0.71- |
| | | | 0.67 (m, 1H), 0.51-0.52 (m, 2H), 0.34-0.33 (m, 1H). |
| 185 | 501a | m/z = | ¹H NMR (400 MHz, DMSO-d6): δ = 10.81 (s, 1H), 8.74 |
| | | 831.5 | (s, 1H), 8.30-8.14 (m, 1H), 8.03 (s, 1H), 7.84 (br d, J = |
| | | [M + H]⁺ | 8.9 Hz, 1H), 7.44 (br d, J = 8.9 Hz, 1H), 7.31 (br d, J = |
| | | | 11.3 Hz, 1H), 6.65 (br d, J = 6.0 Hz, 1H), 6.21-6.08 (m, |
| | | | 1H), 5.18 (br d, J = 3.6 Hz, 1H), 4.53-4.29 (m, 1H), 4.55- |
| | | | 4.23 (m, 1H), 4.22-4.12 (m, 1H), 3.86 (s, 3H), 3.72-3.62 |
| | | | (m, 4H), 3.57 (s, 3H), 2.59 (br d, J = 5.1 Hz, 2H), 2.36- |
| | | | 2.21 (m, 2H), 2.12 (br dd, J = 5.6, 11.9 Hz, 1H), 1.91 (br d, |
| | | | J = 9.1 Hz, 4H), 1.67-1.56 (m, 2H), 1.42-1.21 (m, 4H), |
| | | | 0.78-0.67 (m, 1H), 0.53 (br s, 2H), 0.40-0.32 (m, 1H) |
| 186 | 502a | m/z = | ¹H NMR (400 MHz, DMSO-d6): δ = 10.88 (s, 1H), 8.75 |
| | | 831.4 | (s, 1H), 8.24 (d, J = 2.0 Hz, 1H), 8.03 (s, 1H), 7.83 (br d, J = |
| | | [M + H]⁺ | 9.8 Hz, 1H), 7.44 (d, J = 9.1 Hz, 1H), 6.63 (dd, J = 8.3, |
| | | | 10.3 Hz, 1H), 6.47 (br dd, J = 4.1, 8.2 Hz, 1H), 6.13 (br s, |
| | | | 1H), 4.76 (br s, 1H), 4.53-4.32 (m, 2H), 4.29-4.17 (m, |
| | | | 4H), 3.70-3.62 (m, 3H), 3.56 (s, 3H), 3.26-3.16 (m, 2H), |
| | | | 2.81-2.66 (m, 1H), 2.58-2.52 (m, 2H), 2.29-2.16 (m, |
| | | | 1H), 2.15-2.07 (m, 1H), 1.89 (br d, J = 10.1 Hz, 4H), 1.65- |
| | | | 1.52 (m, 2H), 1.43-1.24 (m, 3H), 0.78-0.65 (m, 1H), |
| | | | 0.59-0.47 (m, 2H), 0.43-0.30 (m, 1H) |
| 187 | 503a | m/z = | ¹H NMR (400 MHz, DMSO-d6): δ (ppm) = 10.84-10.76 |
| | | 827.7 | (m, 1H), 8.85 (s, 1H), 8.00 (s, 1H), 7.93-7.85 (m, 1H), |
| | | [M + H]⁺ | 7.81 (d, J = 2.5 Hz, 1H), 7.59 (dd, J = 2.4, 8.9 Hz, 1H), |
| | | | 7.34-7.23 (m, 2H), 7.13 (s, 1H), 7.01-6.88 (m, 2H), 4.56 |
| | | | (s, 2H), 4.26 (dd, J = 5.1, 9.5 Hz, 1H), 4.17 (s, 3H), 3.56 |
| | | | (br s, 4H), 3.19-3.13 (m, 2H), 2.60 (d, J = 4.6 Hz, 5H), |
| | | | 2.37-2.28 (m, 4H), 2.28-2.22 (m, 1H), 2.18 (br d, J = 6.4 |
| | | | Hz, 2H), 2.15-2.03 (m, 1H), 1.81 (br d, J = 11.8 Hz, 2H), |
| | | | 1.73-1.60 (m, 1H), 1.39-1.23 (m, 2H) |
| 188 | 504a | m/z = | ¹H NMR (400 MHz, DMSO-d6): δ 10.86 (s, 1H), 8.75 (s, |
| | | 871.1 | 1H), 8.18 (d, J = 2.2 Hz, 1H), 8.02 (s, 1H), 7.77-7.70 (m, |
| | | [M + H]⁺ | 1H), 7.59 (d, J = 8.4 Hz, 1H), 7.45 (d, J = 9.1 Hz, 1H), 7.39 |
| | | | (s, 1H), 7.01 (d, J = 8.4 Hz, 1H), 6.17 (s, 1H), 4.48-4.34 |
| | | | (m, 1H), 4.34-4.27 (m, 1H), 4.20-4.03 (m, 2H), 3.95 (s, |
| | | | 3H), 3.56 (s, 3H), 3.29-3.10 (m, 8H), 3.08-2.98 (m, 2H), |
| | | | 2.71-2.54 (m, 2H), 2.39-2.23 (m, 4H), 2.21-2.11 (m, |
| | | | 1H), 1.85-1.67 (m, 4H), 1.59-1.41 (m, 3H), 1.39-1.29 |
| | | | (m, 1H), 0.74-0.65 (m, 1H), 0.57-0.46 (m, 2H), 0.42- |
| | | | 0.31 (m, 1H). |
| 189 | 508a | m/z = | ¹H NMR (400 MHz, DMSO-d6): δ (ppm) = 10.80 (s, 1H), |
| | | 840.7 | 8.76 (s, 1H), 8.18 (d, J = 1.4 Hz, 1H), 8.02 (s, 1H), 7.74 |
| | | [M + H]⁺ | (dd, J = 1.7, 9.1 Hz, 1H), 7.44 (d, J = 9.1 Hz, 1H), 7.30 (d, |
| | | | J = 8.8 Hz, 1H), 6.51 (dd, J = 1.3, 8.9 Hz, 1H), 6.36 (s, |
| | | | 1H), 6.20 |
| | | | (br s, 1H), 5.66 (br d, J = 8.0 Hz, 1H), 4.53-4.29 (m, 2H), |
| | | | 4.16 (dd, J = 5.2, 8.7 Hz, 1H), 3.80 (s, 3H), 3.67-3.51 (m, |
| | | | 7H), 3.26-3.15 (m, 1H), 2.60 (br t, J = 6.6 Hz, 2H), 2.31- |
| | | | 2.08 (m, 2H), 1.88-1.77 (m, 2H), 1.70 (br d, J = 11.4 Hz, |
| | | | 2H), 1.47 (br s, 2H), 1.40-1.19 (m, 7H), 0.77-0.65 (m, |
| | | | 1H), 0.52 (br t, J = 5.7 Hz, 2H), 0.41-0.27 (m, 1H). |
| 190 | 509a | m/z = | ¹H NMR (400 MHz, DMSO-d6): δ 10.90 (s, 1H), 8.90 (s, |
| | | 868.41 | 1H), 8.16 (s, 1H), 8.07-8.06 (m, 1H), 7.75-7.73 (m, 1H), |
| | | [M + H]⁺ | 7.62-7.56 (m, 2H), 7.43 (d, J = 9.20 Hz, 1H), 7.23 (s, 1H), |
| | | | 7.19-6.97 (m, 2H), 6.24 (s, 1H), 4.56-4.35 (m, 6H), 4.19- |
| | | | 4.16 (m, 3H), 3.88-3.79 (m, 1H), 3.66-3.62 (m, 2H), 3.57 |
| | | | (s, 3H), 3.38-3.26 (s, 3H), 3.03-2.59 (m, 5H), 2.49-2.40 (m, |
| | | | 2H), 2.19-2.16 (m, 2H), 1.73-1.69 (m, 2H), 1.33-1.19 (m, |
| | | | 3H), 0.71 (br s, 1H), 0.52-0.51 (m, 2H), 0.35-0.33 (m, 1H). |
| 191 | 511a | m/z = | ¹H NMR (400 MHz, DMSO-d6): δ 10.84 (s, 1H), 8.75 (s |
| | | 885.47 | 1H), 8.19 (d, J = 1.6 Hz, 1H), 8.03 (s, 1H), 7.76 (dd, J = |
| | | [M + H]⁺ | 9.2, 2.0 Hz, 1H), 7.48-7.43 (m, 2H), 7.89 (d, J = 9.2 Hz, |
| | | | 1H), 6.80 (bs, 1H), 6.18 (br s, 1H), 4.56 (br s, 2H), 4.43- |
| | | | 4.40 (m, 2H), 4.26-4.22 (m, 1H), 3.88-3.85 (m, 6H), 3.56 |
| | | | (s, 3H), 3.21 (br s, 1H), 2.64-2.49 (m, 6H), 2.30-2.28 (m, |
| | | | 1H), 2.18-2.16 (m, 1H), 1.76-1.47 (m, 8H), 1.35-1.30 (m, |
| | | | 1H), 1.24-1.21 (m, 2H), 0.91 (s, 3H), 0.71-0.67 (m, 1H), |
| | | | 0.53-0.50 (m, 2H), 0.36-0.35 (m, 1H). |
| 192 | 512a | m/z = | ¹H NMR (400 MHz, DMSO-d6): δ 10.87 (s, 1H), 8.77 (s, |
| | | 914.48 | 1H), 8.27 (s, 1H), 8.18 (br s, 1H), 8.04 (s, 1H), 7.80 (d, J = |
| | | [M + H]⁺ | 8.80 Hz, 1H), 7.37 (dd, J = 6.40, 2.20 Hz, 2H), 6.99-7.01 |
| | | | (m, 2H), 6.20 (s, 1H), 4.50-4.31 (m, 3H), 4.29-4.01 (m, |
| | | | 5H), 3.81-3.51 (m, 5H), 3.13-3.10 (m, 6H), 2.51-2.45 (m, |
| | | | 6H), 2.31-2.27 (m, 1H), 2.19-2.14 (m, 1H), 2.02-1.97 (m, |
| | | | 1H), 1.77-1.74 (m, 2H), 1.42-1.32 (m, 3H), 1.21-0.94 (m, |
| | | | 6H), 0.80-0.68 (m, 1H), 0.60-0.49 (m, 2H), 0.37-0.32 (m, |
| | | | 1H). |
| 193 | 512b | m/z = | ¹H NMR (400 MHz, DMSO-d6): δ 10.80 (s, 1H), 8.76 (s, |
| | | 914.48 | 1H), 8.14-8.04 (m, 3H), 7.81 (dd, J = 7.60, 1.60 Hz, 1H), |
| | | [M + H]⁺ | 7.37 (dd, J = 11.2, 2.00 Hz, 2H), 7.01-6.99 (m, 2H), 6.16 |
| | | | (br s, 1H), 4.50-4.40 (m, 3H), 4.39-4.30 (m, 3H), 4.25 (s, |
| | | | 3H), 4.20-3.60 (m, 3H), 3.55 (s, 1H), 3.30-3.00 (m, 7H), |
| | | | 2.70-2.56 (m, 3H), 2.32-2.27 (m, 1H), 2.17-2.15 (m, 1H), |
| | | | 1.75-1.55 (m, 1H), 1.66-1.43 (m, 3H), 1.06-0.71 (m, 11H), |
| | | | 0.65-0.60 (m, 1H), 0.58-0.0.48 (m, 2H), 0.40-0.33 (m, 1H). |
| 194 | 513a | m/z = | ¹H NMR (400 MHz, DMSO-d6): δ 10.88 (s, 1H), 8.42 (s, |
| | | 853.5 | 1H), 8.13 (s, 1H), 8.05-8.00 (m, 1H), 7.92-7.87 (m, 1H), |
| | | [M + H]⁺ | 7.51-7.38 (m, 3H), 7.14-6.93 (m, 2H), 6.51 (s, 1H), 6.44- |
| | | | 6.38 (m, 1H), 6.29-6.23 (m, 1H), 4.51-4.29 (m, 3H), |
| | | | 4.24 (s, 3H), 4.14-3.89 (m, 2H), 3.57 (s, 3H), 3.26-3.14 |
| | | | (m, 1H), 2.79-2.57 (m, 3H), 2.41-2.22 (m, 1H), 2.22- |
| | | | 2.05 (m, 1H), 2.05-1.53 (m, 1H), 1.46-0.90 (m, 3H), |
| | | | 0.71 (s, 1H), 0.57-0.41 (m, 2H), 0.37-0.25 (m, 1H). |
| 195 | 514a | m/z = | ¹H NMR (400 MHz, DMSO-d6): δ (ppm) = 10.88 (s, 1H), |
| | | 871.4 | 8.18-8.11 (m, 1H), 8.07 (s, 1H), 7.75 (dd, J = 1.8, 9.1 Hz, |
| | | [M + H]⁺ | 1H), 7.59 (d, J = 8.0 Hz, 1H), 7.45 (d, J = 9.1 Hz, 1H), |
| | | | 7.22 (d, J = 7.0 Hz, 1H), 7.16-7.08 (m, 1H), 6.22 (br s, |
| | | | 1H), 5.38 (br s, 1H), 4.53-4.31 (m, 3H), 4.28-4.05 (m, |
| | | | 5H), 3.68 (br d, J = 8.1 Hz, 3H), 3.61-3.20 (m, 16H), 3.14 |
| | | | (br d, J = 3.1 Hz, 1H), 2.75-2.56 (m, 2H), 2.41-2.25 (m, |
| | | | 3H), 2.21-2.11 (m, 2H), 2.08-1.96 (m, 2H), 1.70 (br d, J = |
| | | | 4.9 Hz, 2H), 1.64-1.52 (m, 2H), 1.40-1.27 (m, 1H), |
| | | | 0.78-0.65 (m, 1H), 0.60-0.47 (m, 2H), 0.41-0.29 (m, |
| | | | 1H) |
| 196 | 518a | m/z = | ¹H NMR (400 MHz, DMSO-d6): δ = 10.81 (s, 1H), 8.78 |
| | | 785.7 | (s, 1H), 8.25 (s, 1H), 8.03 (s, 1H), 7.82 (dd, J = 2.0, 9.2 |
| | | [M + H]⁺ | Hz, 1H), 7.43 (d, J = 9.2 Hz, 1H), 7.33 (d, J = 8.8 Hz, 1H), |
| | | | 6.47 (dd, J = 1.6, 8.8 Hz, 1H), 6.27- 6.09 (m, 3H), 4.52- |
| | | | 4.33 (m, 2H), 4.18 (dd, J = 5.2, 8.8 Hz, 1H), 4.07 (s, 2H), |
| | | | 3.91 (s, 2H), 3.80 (s, 4H), 3.57 (s, 3H), 2.72-2.55 (m, 5H), |
| | | | 2.31-2.20 (m, 1H), 2.18-2.10 (m, 1H), 2.08-1.99 (m, |
| | | | 2H), 1.40-1.30 (m, 1H), 0.77-0.67 (m, 1H), 0.53 (d, J = |
| | | | 5.6 Hz, 2H), 0.44-0.33 (m, 1H) |
| 197 | 519a | m/z = | ¹H NMR (400 MHz, DMSO-d6): δ = 10.83 (s, 1H), 8.79- |
| | | 813.3 | 8.50 (m, 1H), 8.30-8.08 (m, 1H), 8.02-7.86 (m, 1H), 7.84- |
| | | [M + H]⁺ | 7.69 (m, 1H), 7.55-7.29 (m, 2H), 7.02-6.52 (m, 1H), |
| | | | 6.45-6.33 (m, 1H), 6.32-6.11 (m, 2H), 4.50-4.36 (m, |
| | | | 1H), 4.23 (dd, J = 5.2, 8.8 Hz, 1H), 3.90-3.74 (m, 3H), |
| | | | 3.65 (d, J = 8.0 Hz, 1H), 3.61-3.47 (m, 6H), 2.60 (d, J = |
| | | | 3.2 Hz, 4H), 2.31-2.20 (m, 2H), 2.19-2.10 (m, 1H), 1.89- |
| | | | 1.73 (m, 3H), 1.62-1.09 (m, 6H), 0.76-0.62 (m, 1H), |
| | | | 0.51 (d, J = 5.6 Hz, 2H), 0.40-0.26 (m, 1H) |
| 198 | 520a | m/z = | ¹H NMR (400 MHz, DMSO-d6): δ = 10.84 (s, 1H), 8.80 |
| | | 799.4 | (s, 1H), 8.42 (s, 1H), 8.26 (d, J = 2.0 Hz, 1H), 8.03 (s, 1H), |
| | | [M + H]⁺ | 7.81 (d, J = 2.0, 9.2 Hz, 1H), 7.44 (d, J = 9.2, 12.8 Hz, |
| | | | 2H), 6.78 (d, J = 1.8, 9.2 Hz, 1H), 6.60 (s, 1H), 6.20 (s, |
| | | | 1H), 4.51-4.34 (m, 2H), 4.24 (d, J = 5.2, 9.2 Hz, 1H), |
| | | | 4.08 (s, 2H), 4.03-3.95 (m, 1H), 3.93-3.88 (m, 2H), 3.86 |
| | | | (s, 3H), 3.57 (s, 3H), 2.82 (s, 3H), 2.66-2.54 (m, 5H), 2.32- |
| | | | 2.11 (m, 4H), 1.40-1.27 (m, 1H), 0.76-0.66 (m, 1H), |
| | | | 0.59-0.47 (m, 2H), 0.45-0.31 (m, 1H) |
| 199 | 521a | m/z = | ¹H NMR (400 MHz, DMSO-d6): δ 10.85 (s, 1H), 9.20 (s, |
| | | 839.48 | 1H), 8.04-8.01 (m, 2H), 7.52-7.44 (m, 3H), 7.21-7.08 (m, |
| | | [M + H]⁺ | 1H), 6.92 (s, 1H), 6.39 (s, 1H), 6.31 (d, J = 2.0 Hz, 1H), |
| | | | 4.51-4.38 (m, 2H), 4.34 (m, 1H), 4.06-4.05 (m, 2H), 3.89- |
| | | | 3.81 (m, 5H), 3.58 (m, 5H), 3.23-3.02 (m, 7H), 2.79 (m, |
| | | | 2H), 2.63-2.50 (m, 2H), 2.16(m, 2H), 1.85-1.82 (m, 3H), |
| | | | 1.39-1.34 (m, 3H), 0.73 (m, 1H), 0.52-0.51 (m, 2H), 0.32 |
| | | | (m, 1H). |
| 200 | 524a | m/z = | ¹H NMR (400 MHz, DMSO-d6): δ = 10.92-10.77 (m, |
| | | 786.5 | 1H), 8.83-8.74 (m, 1H), 8.24 (d, J = 2.0 Hz, 1H), 8.05- |
| | | [M + H]⁺ | 7.99 (m, 1H), 7.81 (dd, J = 2.0, 9.2 Hz, 1H), 7.55 (d, J = |
| | | | 8.8 Hz, 1H), 7.42 (d, J = 9.2 Hz, 1H), 6.85 (s, 1H), 6.67 |
| | | | (dd, J = 2.0, 8.8 Hz, 1H), 6.28-6.10 (m, 1H), 4.83-4.68 |
| | | | (m, 1H), 4.52-4.33 (m, 2H), 4.29 (dd, J = 5.2, 9.6 Hz, |
| | | | 1H), 4.06 (s, 2H), 4.00-3.94 (m, 2H), 3.93-3.88 (m, 3H), |
| | | | 3.56 (s, 3H), 2.88-2.76 (m, 2H), 2.69-2.55 (m, 2H), 2.37- |
| | | | 2.20 (m, 3H), 2.19-2.08 (m, 1H), 2.03-1.87 (m, 1H), |
| | | | 1.40-1.21 (m, 1H), 0.79-0.62 (m, 1H), 0.59-0.45 (m, |
| | | | 2H), 0.43-0.29 (m, 1H) |
| 202 | 527a | m/z = | ¹H NMR (400 MHz, DMSO-d6): δ 10.85 (s, 1H), 8.68 (s, |
| | | 842.41 | 1H), 8.25 (s, 1H), 8.03 (s, 1H), 7.88 (s, 1H), 7.51-7.26 (m, |
| | | [M + H]⁺ | 2H), 6.99-6.67 (m, 2H), 6.13 (s, 1H), 4.44-4.34 (m, 2H), |
| | | | 4.28-4.24 (m, 1H), 3.89 (s, 3H), 3.54-3.22 (m, 8H), 3.21 |
| | | | (m, 8H), 2.67-2.59 (m, 2H), 2.50-2.29 (m, 4H), 2.18-2.15 |
| | | | (m, 1H), 2.07-2.01 (m, 1H), 1.96-1.92 (s, 2H), 1.1.75 (s, |
| | | | 1H), 1.67 (s, 1H), 1.60 (m, 1H), 1.33-1.24 (m, 2H), 0.73- |
| | | | 0.71 (m, 1H) |
| 203 | 527b | m/z = | ¹H NMR (400 MHz, DMSO-d6): δ 10.84 (s, 1H), 8.69 (s, |
| | | 842.36 | 1H), 8.19 (s, 1H), 8.03 (s, 1H), 7.88 (d, J = 9.20 Hz, 1H), |
| | | [M + H]⁺ | 7.50 (d, J = 9.20 Hz, 1H), 7.41 (s, 1H), 6.91 (d, J = 8.80 |
| | | | Hz, 1H), 6.84 (s, 1H), 6.15 (s, 1H), 4.47-4.24 (m, 3H), 3.90 |
| | | | (s, 3H), 3.61-3.55 (m, 5H), 3.21 (brs, 6H), 2.59-2.55 (m, |
| | | | 7H), 2.36-2.33 (m, 3H), 2.14-2.15 (m, 1H), 2.00 (s, 1H), |
| | | | 1.67 (brs, 1H), 1.33-1.23 (m, 2H), 0.71 (t, J = 5.60 Hz, |
| | | | 1H), 0.52 (t, J = 5.60 Hz, 2H), 0.35 (d, J = 5.20 Hz, 1H). |
| 204 | 529a | m/z = | ¹H NMR (400 MHz, DMSO-d6): δ ppm 0.28-0.42 (m, 1H) |
| | | 856.4 | 0.51 (br t, J = 5.94 Hz, 2H) 0.63-0.76 (m, 1H) 0.96 (br |
| | | [M + H]⁺ | d, J = 6.13 Hz, 3H) 1.28-1.40 (m, 1H) 1.74-1.84 (m, 1H) |
| | | | 2.05-2.31 (m, 5H) 2.31-2.35 (m, 1H) 2.34-2.40 (m, 1H) |
| | | | 2.57-2.63 (m, 2H) 2.65-2.69 (m, 1H) 2.79-2.89 (m, |
| | | | 1H) 2.92-3.06 (m, 2H) 3.13-3.27 (m, 3H) 3.34-3.39 |
| | | | (m, 1H) 3.39-3.49 (m, 1H) 3.56 (s, 3H) 3.83 (s, 3H) |
| | | | 3.91-4.05 (m, 2H) 4.18 (s, 1H) 4.30-4.51 (m, 2H) 6.22 |
| | | | (br d, J = 2.63 Hz, 1H) 6.33 (d, J = 1.38 Hz, 1H) 6.51-6.60 |
| | | | (m, 1H) 7.44 (dd, J = 8.94, 4.31 Hz, 2H) 7.71 (dd, J = 9.07, |
| | | | 2.06 Hz, 1H) 8.04 (s, 1H) 8.20 (d, J = 1.88 Hz, 1H) 8.84 |
| | | | (s, 1H) 10.76-10.89 (m, 1H) |
| 205 | 529b | m/z = | ¹H NMR (400 MHz, DMSO-d6): δ ppm 0.51 (br t, J = 5.75 |
| | | 856.5 | Hz, 2H) 0.66-0.75 (m, 1H) 0.94 (br d, J = 6.00 Hz, 3H) |
| | | [M + H]⁺ | 1.28-1.39 (m, 1H) 1.63-1.76 (m, 1H) 2.03-2.39 (m, 8H) |
| | | | 2.57-2.64 (m, 2H) 2.64-2.74 (m, 2H) 2.84-2.95 (m, |
| | | | 2H) 3.04-3.12 (m, 1H) 3.14-3.24 (m, 2H) 3.34-3.46 |
| | | | (m, 2H) 3.55 (s, 3H) 3.83 (s, 3H) 3.95-4.08 (m, 2H) |
| | | | 4.22 (dd, J = 8.76, 5.25 Hz, 1H) 4.30-4.48 (m, 2H) 6.22 |
| | | | (br s, 1H) 6.32 (s, 1H) 6.51-6.57 (m, 1H) 7.44 (dd, |
| | | | J = 9.07, 5.19 Hz, 2H) 7.71 (dd, J = 9.32, 1.81 Hz, 1H) 8.04 |
| | | | (s, 1H) 8.17-8.21 (m, 1H) 8.84 (s, 1H) 10.82 (s, 1H) |
| 206 | 529c | m/z = | ¹H NMR (400 MHz, DMSO-d6): δ ppm 0.31-0.40 (m, 1H) |
| | | 856.4 | 0.46-0.59 (m, 2H) 0.67-0.76 (m, 1H) 0.95 (br d, |
| | | [M + H]⁺ | J = 6.13 Hz, 3H) 1.28-1.39 (m, 1H) 1.63-1.77 (m, 1H) |
| | | | 2.03-2.37 (m, 7H) 2.57-2.74 (m, 4H) 2.80-2.98 (m, 2H) |
| | | | 3.04-3.12 (m, 1H) 3.13-3.25 (m, 2H) 3.35-3.47 (m, |
| | | | 2H) 3.55 (s, 3H) 3.84 (s, 3H) 3.96-4.10 (m, 2H) 4.22 |
| | | | (dd, J = 8.88, 5.13 Hz, 1H) 4.27-4.50 (m, 2H) 6.18-6.25 |
| | | | (m, 1H) 6.32 (s, 1H) 6.49-6.60 (m, 1H) 7.44 (dd, |
| | | | J = 8.82, 7.32 Hz, 2H) 7.69 (dd, J = 9.19, 1.81 Hz, 1H) 8.02- |
| | | | 8.06 (m, 1H) 8.19 (d, J = 1.25 Hz, 1H) 8.84 (s, 1H) |
| | | | 10.78-10.85 (m, 1H) |
| 207 | 530a | m/z = | ¹H NMR (400 MHz, DMSO-d6): δ 10.96 (s, 1H), 8.76 (s, |
| | | 868.35 | 1H), 8.17 (s, 1H), 8.02 (s, 1H), 7.73-7.71 (m, 1H), 7.44 (d, |
| | | [M + H]⁺ | J = 8.80 Hz, 1H), 7.19 (d, J = 7.60 Hz, 1H), 6.98-6.88 (m, |
| | | | 2H), 6.18 (s, 1H), 4.43-4.39 (m, 5H), 4.15-4.01 (m, 5H), |
| | | | 3.56 (s, 3H), 3.21-3.09 (m, 3H), 2.96-2.69 (m, 2H), 2.75- |
| | | | 2.68 (m, 5H), 2.36-2.12 (m, 4H), 1.98-1.87 (m, 2H), 1.75- |
| | | | 1.66 (m, 2H), 1.01-0.99 (m, 3H), 0.72-. 070 (m, 1H), 0.52- |
| | | | 0.49 (m, 2H), 0.35-0.33 (m, 1H). |
| 208 | 532a | m/z = | ¹H NMR (400 MHz, DMSO-d6): δ 10.85 (s, 1H), 8.88 (s, |
| | | 871.3 | 1H), 8.20 (s, 1H), 8.08 (s, 1H), 7.70 (d, J = 9.2 Hz, 1H), |
| | | [M + H]⁺ | 7.52 (d, J = 9.2 Hz, 1H), 7.45 (d, J = 9.2 Hz, 1H), 6.94- |
| | | | 6.88 (m, 2H), 6.25 (br s, 1H), 4.50-4.24 (m, 3H), 3.89 (s, |
| | | | 3H). 3.70-3.56 (m, 7H), 3.40-3.10 (m, 11H), 2.70-2.55 (m, |
| | | | 3H), 2.40-2.25 (m, 1H), 2.20-2.08 (m, 2H), 1.40-1.30 (m, |
| | | | 1H), 0.75-0.65 (m, 1H), 0.55-0.45 (m, 2H), 0.40-0.28 (m, |
| | | | 1H) |
| 209 | 534a | m/z = | ¹H NMR (400 MHz, DMSO-d6): δ = 10.86-10.75 (m, |
| | | 881.3 | 1H), 8.82-8.75 (m, 1H), 8.26-8.11 (m, 1H), 8.03 (s, 1H), |
| | | [M + H]⁺ | 7.89-7.78 (m, 1H), 7.43 (d, J = 9.2 Hz, 1H), 7.34 (d, J = |
| | | | 8.8 Hz, 1H), 6.67 (d, J = 8.8 Hz, 1H), 6.48 (s, 1H), 6.32- |
| | | | 6.14 (m, 1H), 5.78 (d, J = 3.2, 6.4 Hz, 1H), 4.52-4.29 (m, |
| | | | 2H), 4.18 (dd, J = 5.2, 8.8 Hz, 1H), 4.13-4.00 (m, 1H), |
| | | | 3.84-3.72 (m, 4H), 3.71-3.63(m, 2H), 3.57 (s, 3H), 2.69- |
| | | | 2.56 (m, 3H), 2.54 (s, 2H), 2.35-2.12 (m, 3H), 1.92 (d, J = |
| | | | 13.2 Hz, 1H), 1.86-1.73 (m, 2H), 1.70-1.59 (m, 1H), 1.54- |
| | | | 1.41 (m, 1H), 1.40-1.30 (m, 1H), 0.78-0.63 (m, 1H), |
| | | | 0.63-0.43 (m, 2H), 0.42-0.29 (m, 1H) |
| 210 | 534b | m/z = | ¹H NMR (400 MHz, DMSO-d6): δ = 10.83 (s, 1H), 8.81 |
| | | 881.4 | (s, 1H), 8.42 (s, 1H), 8.21 (br s, 1H), 8.05 (s, 1H), 7.83 (br |
| | | [M + H]⁺ | d, J = 8.6 Hz, 1H), 7.44 (d, J = 9.1 Hz, 1H), 7.32 (d, J = |
| | | | 8.4 Hz, 1H), 6.51-6.39 (m, 2H), 6.20 (br d, J = 0.9 Hz, |
| | | | 1H), 5.89-5.43 (m, 1H), 4.54-4.31 (m, 2H), 4.18 (dd, J = |
| | | | 5.3, 8.6 Hz, 1H), 3.81 (s, 3H), 3.74-3.61 (m, 4H), 3.58 (s, |
| | | | 3H), 3.24 (br dd, J = 6.3, 11.4 Hz, 2H), 2.65-2.55 (m, |
| | | | 2H), 2.47-2.36 (m, 1H), 2.30-2.09 (m, 3H), 2.01 (br d, J = |
| | | | 12.4 Hz, 1H), 1.93-1.84 (m, 1H), 1.75-1.57 (m, 2H), |
| | | | 1.38-1.29 (m, 1H), 1.28-1.16 (m, 1H), 0.72 (br s, 1H), |
| | | | 0.53 (br d, J = 5.5 Hz, 2H), 0.36 (br d, J = 5.1 Hz, 1H) |
| 211 | 535a | m/z = | ¹H NMR (400 MHz, DMSO-d6): δ = 10.88 (s, 1H), 8.74 |
| | | 831.5 | (s, 1H), 8.19 (d, J = 2.0 Hz, 1H), 8.09-7.97 (m, 1H), 7.83 |
| | | [M + H]⁺ | (dd, J = 2.4, 9.2 Hz, 1H), 7.43 (d, J = 9.2 Hz, 1H), 7.31 |
| | | | (dd, J = 4.8, 8.8 Hz, 1H), 6.95 (dd, J = 9.2, 10.4 Hz, 1H), |
| | | | 6.22-6.07 (m, 1H), 4.49-4.28 (m, 3H), 4.19 (s, 3H), 3.74- |
| | | | 3.62 (m, 2H), 3.59-3.55 (m, 5H), 3.26-3.18 (m, 1H), |
| | | | 2.96-2.85 (m, 1H), 2.67-2.59 (m, 3H), 2.33 (dd, J = 5.2, |
| | | | 9.2 Hz, 1H), 2.15 (d, J = 5.2, 8.0 Hz, 1H), 1.85-1.77 (m, |
| | | | 2H), 1.57 (d, J = 6.4 Hz, 1H), 1.46-1.30 (m, 5H), 0.76- |
| | | | 0.68 (m, 1H), 0.52 (t, J = 6.0 Hz, 2H), 0.40-0.32 (m, 1H) |
| 212 | 537a | m/z = | ¹H NMR (400 MHz, DMSO-d6): δ 10.87 (s, 1H), 8.88 (s, |
| | | 870.4 | 1H), 8.22 (s, 1H), 8.07 (s, 1H), 7.73-7.65 (m, 1H), 7.65- |
| | | [M + H]⁺ | 7.57 (m, 1H), 7.49-7.39 (m, 2H), 7.07-6.98 (m, 1H), |
| | | | 6.26 (s, 1H), 4.51-4.28 (m, 3H), 3.97 (s, 3H), 3.82-3.69 |
| | | | (m, 2H), 3.67-3.52 (m, 7H), 3.25-3.11 (m, 5H), 2.94- |
| | | | 2.76 (m, 3H), 2.72-2.54 (m, 2H), 2.41-2.09 (m, 2H), |
| | | | 1.86-1.60 (m, 4H), 1.39-1.27 (m, 1H), 0.75-0.62 (m, |
| | | | 1H), 0.58-0.42 (m, 2H), 0.35 (s, 1H). |
| 213 | 540a | m/z = | ¹H NMR (400 MHz, DMSO-d6): δ 10.90 (s, 1H), 8.91 (s, |
| | | 871.33 | 1H), 8.22 (s, 1H), 8.08 (s, 1H), 7.70 (d, J =7.6 Hz, 1H), |
| | | [M + H]⁺ | 7.46-7.42 (m, 2H), 7.04 (s, 2H), 6.28 (s, 1H), 4.44- 4.34 |
| | | | (m, 3H), 4.25 (s, 3H), 3.62-3.57 (m, 10H), 3.22-3.17 (m, |
| | | | 10H), 2.80 (brs, 2H), 2.67-2.60 (m, 1H) 2.18-2.17 (m, 1H), |
| | | | 1.35 (s, 1H), 0.72-0.71 (m, 1H), 0.51-0.49 (m, 2H), 0.37- |
| | | | 0.35 (m, 1H) |
| 214 | 542a | m/z = | ¹H NMR (400 MHz, DMSO-d6): δ 13.0 (br s, 1H), 10.84 |
| | | 912.38 | (s, 1H), 8.84 (s, 1H), 8.18 (d, J = 24.00 Hz, 2H), 8.04 (s, |
| | | [M + H]⁺ | 1H), 7.69 (d, J = 8.40 Hz, 1H), 7.48-7.42 (m, 1H), 6.91- |
| | | | 6.84 (m, 2H), 6.22 (s, 1H), 4.41-4.39 (m, 2H), 4.26-4.24 |
| | | | (m, 1H), 3.88-3.83 (m, 4H), 3.56-3.48 (m, 11H), 3.31-3.30 |
| | | | (m, 2H), 2.94-2.92 (m, 3H), 2.66-2.60 (m, 1H), 2.50-2.41 |
| | | | (m, 2H), 2.41-2.40 (m, 2H), 2.33-2.13 (m, 8H), 1.96-1.88 |
| | | | (m, 3H), 1.75 (m, 1H), 1.68-1.65 (m, 1H), 1.58-1.51 (m, |
| | | | 2H), 1.37-1.35 (m, 3H), 1.11-1.08 (m, 1H), 0.72-0.69 (m, |
| | | | 1H), 0.51-0.49 (m, 2H), 0.39-0.35 (m, 1H). |
| 215 | 543a | m/z = | ¹H NMR (400 MHz, DMSO-d6): δ 10.78 (s, 1H), 8.76 (s, |
| | | 447.3 | 1H), 8.11 (s, 1H), 8.00 (s, 1H), 7.66 (d, J = 8.9 Hz, 1H), |
| | | [M/2 + H]⁺ | 7.50 (d, J = 8.9 Hz, 1H), 7.37 (d, J = 8.9 Hz, 1H), 7.05- |
| | | | 6.92 (m, 2H), 6.12 (s, 1H), 4.52 (s, 2H), 4.38 (s, 4H), 4.27- |
| | | | 4.16 (m, 1H), 4.11-3.99 (m, 2H), 3.86 (s, 3H), 3.78- |
| | | | 3.67 (m, 2H), 3.48 (s, 3H), 3.22-3.09 (m, 3H), 3.07-2.85 |
| | | | (m, 2H), 2.65-2.49 (m, 3H), 2.26-2.01 (m, 2H), 1.87- |
| | | | 1.75 (m, 2H), 1.69-1.56 (m, 2H), 1.27-1.16 (m, 2H), |
| | | | 0.71-0.55 (m, 1H), 0.47-0.38 (m, 2H), 0.29-0.20 (m, |
| | | | 1H). |
| 216 | 544a | m/z = | ¹H NMR (400 MHz, DMSO-d6): δ 10.77 (s, 1H), 8.99 (s, |
| | | 440.8 | 1H), 8.14 (s, 1H), 8.08 (s, 1H), 7.64 (d, J = 9.1 Hz, 1H), |
| | | [M/2 + H]⁺ | 7.42 (t, J = 9.8 Hz, 2H), 6.88 (d, J = 9.1 Hz, 1H), 6.81 (s, |
| | | | 1H), 6.21 (s, 1H), 4.94-4.66 (m, 1H), 4.44-4.31 (m, 1H), |
| | | | 4.19 (dd, J = 9.2, 5.0 Hz, 1H), 4.07-3.88 (m, 3H), 3.85- |
| | | | 3.73 (m, 6H), 3.52 (s, 3H), 2.91-2.76 (m, 3H), 2.57-2.51 |
| | | | (m, 1H), 2.27-2.16 (m, 2H), 2.13-2.04 (m, 1H), 1.99- |
| | | | 1.86 (m, 2H), 1.85-1.70 (m, 2H), 1.17 (s, 3H), 0.82-0.73 |
| | | | (m, 0H), 0.68-0.56 (m, 1H), 0.48-0.38 (m, 2H), 0.29- |
| | | | 0.18 (m, 1H). |
| 217 | 546a | m/z = | ¹H NMR (400 MHz, DMSO-d6): δ 10.78 (s, 1H), 8.76 (s, |
| | | 440.8 | 1H), 8.11 (s, 1H), 8.00 (s, 1H), 7.66 (d, J = 8.8 Hz, 1H), |
| | | [M/2 + H]⁺ | 7.50 (d, J = 8.9 Hz, 1H), 7.37 (d, J = 8.9 Hz, 1H), 7.05- |
| | | | 6.95 (m, 2H), 6.12 (s, 1H), 4.52 (s, 2H), 4.44-4.29 (m, |
| | | | 4H), 4.29-4.17 (m, 1H), 4.09-4.01 (m, 2H), 3.86 (s, 3H), |
| | | | 3.77-3.69 (m, 2H), 3.48 (s, 3H), 3.22-3.10 (m, 1H), 3.04- |
| | | | 2.88 (m, 2H), 2.62-2.52 (m, 3H), 2.14-2.04 (m, 1H), |
| | | | 1.85-1.77 (m, 2H), 1.66-1.53 (m, 2H), 1.27-1.15 (m, |
| | | | 2H), 0.64-0.58 (m, 1H), 0.48-0.36 (m, 2H), 0.32-0.19 |
| | | | (m, 1H). |
| 218 | 548b | m/z = | ¹H NMR (400 MHz, DMSO-d6): δ 10.83 (s, 1H), 8.86 (s, |
| | | 842.71 | 1H), 8.22 (d, J = 1.60 Hz, 1H), 8.04 (d, J = 6.80 Hz, 1H), |
| | | [M + H]⁺ | 7.69 (dd, J = 2.00, 9.00 Hz, 1H), 7.44 (t, J = 8.80 Hz, 2H), |
| | | | 6.54 (d, J = 9.20 Hz, 1H), 6.33 (s, 1H), 6.24 (br, 1H), 4.41- |
| | | | 4.39 (m, 2H), 4.22-4.21 (m, 1H), 3.84 (s, 3H), 3.60-3.55 |
| | | | (m, 6H), 3.48-3.46 (m, 1H), 3.32-3.22 (m, 3H), 3.07-3.05 |
| | | | (m, 1H), 2.67-2.60 (m, 3H), 2.52-2.50 (m, 6H), 2.44-2.43 |
| | | | (m, 1H), 2.39-2.38 (m, 2H), 1.70-1.67 (m, 1H), 1.49-1.48 |
| | | | (m, 2H), 0.79-0.67 (m, 1H), 0.59-0.49 (m, 2H), 0.48-0.39 |
| | | | (m, 1H). |
| 219 | 552a | m/z = | ¹H NMR (400 MHz, DMSO-d6): δ (ppm) = 10.84 (s, 1H), |
| | | 441.9 | 8.79 (s, 1H), 8.18 (d, J = 1.6 Hz, 1H), 8.02 (s, 1H), 7.71 |
| | | [M/2 + H]⁺ | (dd, J = 1.8, 9.1 Hz, 1H), 7.49 (br d, J = 9.0 Hz, 1H), 7.43 |
| | | | (d, J = 9.1 Hz, 1H), 6.91 (br d, J = 8.1 Hz, 1H), 6.83 (br s, |
| | | | 1H), 6.21 (br s, 1H), 4.52-4.31 (m, 2H), 4.26 (br dd, J = |
| | | | 5.1, 8.9 Hz, 1H), 3.89 (s, 3H), 3.56 (s, 5H), 3.53-3.47 (m, |
| | | | 2H), 3.27-3.14 (m, 4H), 2.72 (br s, 3H), 2.46-2.35 (m, |
| | | | 3H), 2.35-2.24 (m, 1H), 2.22-2.10 (m, 1H), 2.06-1.93 |
| | | | (m, 2H), 1.67-1.49 (m, 4H), 1.49-1.39 (m, 2H), 1.39- |
| | | | 1.25 (m, 1H), 0.77-0.65 (m, 1H), 0.58-0.45 (m, 2H), 0.40- |
| | | | 0.30 (m, 1H) |
| 220 | 553a | m/z = | ¹H NMR (400 MHz, DMSO-d6): δ (ppm) = 10.87 (s, 1H), |
| | | 441.9 | 8.79 (s, 1H), 8.19 (d, J = 1.6 Hz, 1H), 8.02 (s, 1H), 7.71 |
| | | [M/2 + H]⁺ | (dd, J = 1.8, 9.0 Hz, 1H), 7.44 (d, J = 9.1 Hz, 1H), 7.42- |
| | | | 7.35 (m, 1H), 7.04 (br s, 2H), 6.21 (br s, 1H), 4.52-4.29 |
| | | | (m, 3H), 4.23 (s, 3H), 3.64-3.54 (m, 5H), 3.51 (br d, J = |
| | | | 1.4 Hz, 2H), 3.28-3.02 (m, 3H), 2.97-2.74 (m, 4H), 2.72- |
| | | | 2.55 (m, 3H), 2.39-2.26 (m, 1H), 2.24-1.93 (m, 5H), |
| | | | 1.69-1.50 (m, 4H), 1.49-1.39 (m, 2H), 1.39-1.28 (m, |
| | | | 1H), 0.78-0.66 (m, 1H), 0.58-0.45 (m, 2H), 0.41-0.30 |
| | | | (m, 1H) |
| 221 | 554a | m/z = | 1H NMR (400 MHz, DMSO-d6): δ 10.93-10.88 (m, 1H), |
| | | 888.4 | 10.18-10.14 (m, 1H), 8.82-8.77 (m, 1H), 8.16-8.12 |
| | | [M + H]⁺ | (m, 1H), 8.05-8.01 (m, 1H), 7.80-7.73 (m, 1H), 7.51- |
| | | | 7.41 (m, 2H), 6.99-6.95 (m, 1H), 6.25-6.20 (m, 1H), |
| | | | 4.57-4.22 (m, 1H), 4.11-4.07 (m, 3H), 3.75-3.44 (m, |
| | | | 4H), 3.23-2.91 (m, 3H), 2.32-1.87 (m, 6H), 1.62 (s, 1H), |
| | | | 1.46 (s, 1H), 1.39-1.21 (m, 3H), 0.91-0.64 (m, 4H), 0.55- |
| | | | 0.51 (m, 3H), 0.38-0.33 (m, 1H). |
| 222 | 555a | m/z = | ¹H NMR (400 MHz, DMSO-d6): δ 10.91-10.76 (m, 1H), |
| | | 882.5 | 8.82-8.68 (m, 1H), 8.24-8.11 (m, 2H), 8.08-7.99 (m, |
| | | [M + H]⁺ | 1H), 7.85-7.67 (m, 1H), 7.52-7.36 (m, 3H), 7.08-6.56 |
| | | | (m, 2H), 4.54-4.29 (m, 3H), 4.29-4.19 (m, 2H), 3.94- |
| | | | 3.82 (m, 3H), 3.54 (s, 2H), 3.17 (s, 3H), 2.90-2.56 (m, |
| | | | 5H), 2.37-1.75 (m, 9H), 1.71-1.49 (m, 2H), 1.31 (d, J = |
| | | | 61.8 Hz, 2H), 1.14-1.01 (m, 1H), 0.75-0.62 (m, 1H), |
| | | | 0.60-0.42 (m, 2H), 0.40-0.27 (m, 1H). |
| 223 | 556a | m/z = | ¹H NMR (400 MHz, DMSO-d6): δ 10.88-10.82 (m, 1H), |
| | | 884.5 | 8.86 (d, J = 5.2 Hz, 1H), 8.25-8.17 (m, 1H), 8.13 (s, 1H), |
| | | [M + H]⁺ | 8.08-8.04 (m, 1H), 7.78-7.64 (m, 1H), 7.55-7.34 (m, |
| | | | 3H), 6.93-6.78 (m, 2H), 6.30-6.09 (m, 1H), 5.75 (s, 1H), |
| | | | 4.52-4.20 (m, 6H), 4.17-3.93 (m, 1H), 3.93-3.83 (m, |
| | | | 4H), 3.54 (s, 3H), 3.17 (s, 5H), 3.07-2.90 (m, 3H), 2.66- |
| | | | 2.57 (m, 2H), 2.36-2.01 (m, 4H), 1.86-1.71 (m, 1H), |
| | | | 1.58-1.42 (m, 1H), 1.42-1.22 (m, 2H), 0.77-0.60 (m, |
| | | | 1H), 0.58-0.40 (m, 3H), 0.39-0.24 (m, 2H). |
| 224 | 557a | m/z = | ¹H NMR (400 MHz, DMSO-d6): δ 10.84 (s, 1H), 8.79- |
| | | 868.5 | 8.55 (m, 1H), 8.28-7.95 (m, 4H), 7.83-7.64 (m, OH), |
| | | [M + H]⁺ | 7.53-7.40 (m, 2H), 6.95-6.88 (m, 1H), 6.83 (s, 1H), 6.24- |
| | | | 5.92 (m, 1H), 4.50-4.29 (m, 3H), 4.29-4.20 (m, 1H), |
| | | | 3.89 (s, 3H), 3.55 (s, 3H), 3.26-3.15 (m, 5H), 3.03 (d, J = |
| | | | 51.9 Hz, 1H), 2.72-2.53 (m, 7H), 2.41-2.10 (m, 5H), |
| | | | 1.69 (s, 1H), 1.43-1.20 (m, 2H), 0.94-0.42 (m, 5H), 0.35 |
| | | | (s, 1H), 0.21 (s, 1H). |
| 225 | 558a | m/z = | ¹H NMR (400 MHz, DMSO-d6): δ 10.89 (s, 1H), 8.87 (s, |
| | | 869.43 | 1H), 8.17 (s, 1H), 8.15 (s, 1H), 7.73-7.72 (m, 1H), 7.67- |
| | | [M + H]⁺ | 7.66 (m, 1H), 7.44 (d, J = 9.20 Hz, 1H), 7.38 (s, 1H), 7.10 |
| | | | (s, 1H), 6.24 (s, 1H), 4.33-4.32 (m, 1H), 4.07 (s, 2H), 3.54 |
| | | | (s, 3H), 2.95 (s, 2H), 2.62 (t, J = 5.20 Hz, 3H), 2.18 (t, J = |
| | | | 6.00 Hz, 1H), 2.14 (s, 4H), 1.94 (t, J = 14.40 Hz, 3H), 1.49 |
| | | | (m, 5H), 1.36 (m, 4H), 1.32 (m, 3H), 1.24 (t, J = Hz, 3H), |
| | | | 1.20 (t, J = Hz, 3H), 0.71 (m, 1H), 0.53-0.51 (m, 2H), |
| | | | 0.34-0.33 (m,1H). |
| 226 | 559a | m/z = | ¹H NMR (400 MHz, DMSO-d6): δ 10.88 (s, 1H), 8.77 (s, |
| | | 886.48 | 1H), 8.17 (s, 1H), 8.03 (s, 1H), 7.75 (dd, J = 1.60, 9.20 Hz, |
| | | [M + H]⁺ | 1H), 7.45 (d, J = 9.20 Hz, 1H), 7.39 (d, J = 7.60 Hz, 1H), |
| | | | 7.02 (d, J = 7.20 Hz, 2H), 6.18 (br s, 1H), 4.35-4.34 (m, |
| | | | 6H), 4.23 (s, 3H), 3.57 (s, 3H), 3.10-3.00 (m, 6H), 2.81- |
| | | | 2.78 (m, 2H), 2.62-2.61 (m, 4H), 1.84 (s, 2H), 1.75 (s, 5H), |
| | | | 1.36 (s, 5H), 0.85-0.83 (m, 1H), 0.72-0.70 (m, 1H), 0.55- |
| | | | 0.50 (m, 2H), 0.35-0.33 (m, 1H). |
| 227 | 559b | m/z = | ¹H NMR (400 MHz, DMSO-d6): δ 10.90 (s, 1H), 8.77 (s, |
| | | 886.48 | 1H), 8.20 (d, J = 1.60 Hz, 1H), 8.03 (s, 1H), 7.74 (d, J = |
| | | [M + H]⁺ | 7.60 Hz, 1H), 7.45 (d, J = 9.20 Hz, 1H), 7.39 (d, J = 7.60 |
| | | | Hz, 1H), 7.03-7.01 (m, 2H), 6.18 (s, 1H), 4.45-4.31 (m, |
| | | | 5H), 4.23 (s, 3H), 3.58 (s, 1H), 3.57 (s, 3H), 3.0 (brs, 3H), |
| | | | 2.76-2.70 (m, 3H), 2.62-2.61 (m, 4H), 2.40 (s, 1H), 2.15- |
| | | | 2.14 (m, 1H), 1.87 (s, 6H), 1.75-1.60 (m, 3H), 1.36-1.34 |
| | | | (m, 2H), 1.03-1.00 (m, 1H), 0.72-0.69 (t, J = 6.00 Hz, 1H), |
| | | | 0.53-0.50 (t, J = 6.00 Hz, 2H), 0.35 (d, J = 5.20 Hz, 1H). |
| 228 | 560a | m/z = | ¹H NMR (400 MHz, DMSO-d6): δ 10.90 (s, 1H), 8.76 (s, |
| | | 886.50 | 1H), 8.18 (d, J = 1.60 Hz, 1H), 8.03 (s, 1H), 7.74 (d, J = |
| | | [M + H]⁺ | 8.00 Hz, 1H), 7.47 (q, J = 9.20 Hz, 2H), 6.89 (d, J = 9.20 |
| | | | Hz, 1H), 6.80 (s, 1H), 6.17 (s, 1H), 4.44 (s, 1H), 4.42-4.40 |
| | | | (m, 3H), 4.25-4.24 (m, 1H), 3.89 (s, 3H), 3.63 (s, 1H), 3.56 |
| | | | (s, 3H), 3.46-3.32 (m, 3H), 3.23 (s, 1H), 2.89-2.86 (m, 3H), |
| | | | 2.59-2.51 (m, 2H), 2.37-2.29 (m, 2H), 2.14-2.12 (m, 2H), |
| | | | 1.85 (s, 3H), 1.75-1.60 (m, 3H), 1.40 (s, 1H), 1.24 (s, 2H), |
| | | | 1.10-0.90 (m, 2H), 0.73 (s, 1H), 0.54 (t, J = 6.00 Hz, 2H), |
| | | | 0.37 (d, J = 5.60 Hz, 1H). |
| 229 | 562a | m/z = | ¹H NMR (400 MHz, DMSO-d6): § 10.85 (s, 1H), 8.78 (s, |
| | | 870.40 | 1H), 8.18 (s, 1H), 8.03 (s, 1H), 7.72 (d, J = 9.2 Hz, 1H), |
| | | [M + H]⁺ | 7.53 (d, J = 8.8 Hz, 1H), 7.43 (d, J = 9.2 Hz, 1H), 6.91 (d, |
| | | | J = 9.2 Hz, 1H), 6.86 (s, 1H), 6.20 (br s, 1H), 4.50-4.30 (m, |
| | | | 4H), 3.95-3.85 (m, 5H), 3.55 (s, 5H), 3.47 (br s, 2H), 3.20- |
| | | | 3.10 (br s, 3H), 2.81-2.69 (m, 2H), 2.62-2.55 (m, 2H), |
| | | | 2.35-2.30 (m, 1H), 2.22-2.11 (m, 1H), 2.07 (s, 1H), 1.98- |
| | | | 1.88 (m, 1H), 1.62-1.52 (m, 2H), 1.35-1.25 (m, 1H), 1.15- |
| | | | 1.02 (m, 2H), 0.75-0.65 ( m,1H), 0.55-0.45 (m, 2H), 0.40- |
| | | | 0.30 (m, 1H). |
| 230 | 566a | m/z = | ¹H NMR (400 MHz, DMSO-d6): δ = 10.84 (s, 1H), 8.72 |
| | | 897.3 | (s, 1H), 8.21 (s, 1H), 8.17 (s, 1H), 8.01 (s, 1H), 7.90-7.82 |
| | | [M + H]⁺ | (m, 1H), 7.45 (dd, J = 9.0, 14.5 Hz, 2H), 6.89 (dd, J = 1.6, |
| | | | 9.0 Hz, 1H), 6.83 (d, J = 1.4 Hz, 1H), 6.10 (br s, 1H), 4.40 |
| | | | (br s, 2H), 4.24 (dd, J = 5.1, 9.3 Hz, 1H), 3.88 (s, 3H), 3.67- |
| | | | 3.57 (m, 6H), 3.56 (s, 3H), 2.96 (br t, J = 9.6 Hz, 2H), |
| | | | 2.72-2.52 (m, 3H), 2.38-2.21 (m, 2H), 2.20-2.06 (m, |
| | | | 1H), 1.95-1.78 (m, 4H), 1.70 (br s, 2H), 1.59-1.44 (m, |
| | | | 4H), 1.59-1.22 (m, 4H), 0.78-0.65 (m, 1H), 0.52 (br t, J = |
| | | | 5.8 Hz, 2H), 0.41-0.25 (m, 1H) |
| 257 | 541a | m/z = | ¹H NMR (400 MHz, DMSO-d6): δ 10.89 (s, 1H), 8.89 (s, |
| | | 870.31 | 1H), 8.22 (s, 1H), 8.07 (s, 1H), 7.70 (d, J = 4.80 Hz, 1H), |
| | | [M + H]⁺ | 7.68 (d, J = 8.40 Hz, 1H), 7.56 (d, J = 8.40 Hz, 1H), 7.46 |
| | | | (d, J = 8.40 Hz, 1H), 7.24 (d, J = 8.40 Hz, 1H), 7.07 (t, J = |
| | | | 8.40 Hz, 1H), 6.28 (s, 1H), 4.44-4.33 (m, 3H), 4.22 (s, 3H), |
| | | | 3.78 (d, J = 8.40 Hz, 2H), 3.70-3.50 (m, 8H), 3.20 (br s, |
| | | | 5H), 3.01-2.95 (t, J = 8.40 Hz, 2H), 2.64-2.61 (m, 2H), 2.39 |
| | | | (s, 1H), 2.17-2.07 (m, 1H), 1.89-1.86 (m, 2H), 1.71-1.68 |
| | | | (m, 2H), 0.75-0.65 (s, 1H), 0.55-0.45 (m, 2H), 0.40-0.30 |
| | | | (m, 1H). |
| 258 | 568a | m/z = | ¹H NMR (400 MHz, DMSO-d6): δ 10.87 (s, 1H), 8.67 (s, |
| | | 870.53 | 1H), 8.19 (s, 1H), 7.81 (s, 1H), 7.42 (d, J = 9.20 Hz, 1H), |
| | | [M + H]⁺ | 7.38 (d, J = 8.80 Hz, 1H), 7.02-6.91 (m, 2H), 6.12 (s, 1H), |
| | | | 4.45-4.33 (m, 3H), 4.21 (s, 3H), 3.89-3.53 (m, 7H), 3.15- |
| | | | 2.78 (m, 6H), 2.69-2.57 (m, 4H), 2.30-2.08 (m, 6H), 1.97- |
| | | | 1.45 (m, 5H), 1.37-1.15 (m, 2H), 1.03 (s, 1H), 0.74-0.65 |
| | | | (m, 1H), 0.54-0.51 (m, 2H), 0.32 (s, 1H). |
| 259 | 568b | m/z = | ¹H NMR (400 MHz, DMSO-d6): δ 10.87 (s, 1H), 8.68 (s, |
| | | 870.50 | 1H), 8.18 (s, 1H), 8.03 (s, 1H), 7.84-7.81 (m, 1H), 7.42 (d, |
| | | [M + H]⁺ | J = 9.20 Hz, 1H), 7.38 (d, J = 8.80 Hz, 1H), 7.03-6.93 (m, |
| | | | 2H), 6.15 (s, 1H), 4.45-4.31 (m, 3H), 4.21 (s, 3H), 3.94- |
| | | | 3.42 (m, 7H), 3.10-2.70 (m, 6H), 2.67-2.59 (m, 3H), 2.31- |
| | | | 2.10 (m, 6H), 1.98-1.73 (m, 3H), 1.68-1.48 (m, 2H), 1.38- |
| | | | 1.20 (m, 2H), 1.03 (br s, 1H), 0.73-0.62 (m, 1H), 0.54-0.51 |
| | | | (m, 2H), 0.34 (s, 1H). |
| 260 | 569a | m/z = | ¹H NMR (400 MHz, DMSO-d6): δ 10.81 (s, 1H), 8.74 (s, |
| | | 870.57 | 1H), 8.16 (s, 1H), 8.01 (s, 1H), 7.73 (d, J = 8.80 Hz, 1H), |
| | | [M + H]⁺ | 7.43 (dd, J = 4.40, 9.00 Hz, 2H), 6.70 (d, J = 8.40 Hz, 1H), |
| | | | 6.51 (s, 1H), 6.15 (brs, 1H), 4.40-4.39 (m, 4H), 4.20-4.19 |
| | | | (m, 1H), 3.83 (s, 3H), 3.61 (s, 5H), 3.19-3.18 (brs, 1H), |
| | | | 2.73-2.72 (m, 4H), 2.52-2.50 (m, 4H), 2.27-2.26 (m, 3H), |
| | | | 2.18-2.16 (m, 1H), 1.91-1.88 (m, 2H), 1.69-1.66 (m, 3H), |
| | | | 1.34-1.33 (m, 2H), 1.24 (s, 1H), 0.96 (d, J = 11.60 Hz, 2H), |
| | | | 0.70 (s, 1H), 0.51-0.50 (m, 2H), 0.35-0.34 (m, 1H). |
| 261 | 574a | m/z = | ¹H NMR (400 MHz, DMSO-d6): δ 10.87 (s, 1H), 8.76 (s, |
| | | 870.50 | 1H), 8.18 (d, J = 2.00 Hz, 1H), 8.03 (s, 1H), 7.74 (dd, J = |
| | | [M + H]⁺ | 2.00, 9.20 Hz, 1H), 7.45 (d, J = 9.20 Hz, 1H), 7.37 (d, J = |
| | | | 8.00 Hz, 1H), 7.10 (d, J = 7.20 Hz, 1H), 7.00-6.99 (m, 1H), |
| | | | 6.18 (brs, 1H), 4.33-4.32 (m, 5H), 4.28 (s, 3H), 3.57 (s, |
| | | | 3H), 3.20-3.16 (m, 4H), 2.76-2.75 (m, 6H), 2.62-2.61 (m, |
| | | | 2H), 2.33-2.32 (m, 3H), 2.18-2.17 (m, 1H), 1.91-1.90 (m, |
| | | | 2H), 1.76-1.73 (m, 3H), 1.36 (m, 2H), 1.03-1.00 (m, 2H), |
| | | | 0.69-0.68 (m, 1H), 0.51-0.50 (m, 2H), 0.36-0.34 (m, 1H). |
| 262 | 570a | m/z = | ¹H NMR (400 MHz, DMSO-d6): δ 10.86 (s, 1H), 8.77 (s, |
| | | 843.47 | 1H), 8.30 (s, 1H), 8.15 (d, J = 1.60 Hz, 1H), 8.02 (s, 1H), |
| | | [M + H]⁺ | 7.73 (dd, J = 10.80, Hz, 1H), 7.57 (d, J = 8.80 Hz, 1H), |
| | | | 7.43 (d, J = 9.20 Hz, 1H), 6.85 (d, J = 4.00 Hz, 1H), 6.68 |
| | | | (dd, J = 20.00, Hz, 1H), 6.19 (s, 1H), 4.86 (t, J = Hz, 1H), |
| | | | 4.44-4.28 (m, 5H), 3.91 (s, 3H), 3.82 (t, J = 13.60 Hz, 2H), |
| | | | 3.56 (s, 3H), 2.96 (t, J = 12.80 Hz, 2H), 2.73 (t, J = 12.80 |
| | | | Hz, 2H), 2.65-2.59 (m, 2H), 2.31 (s, 3H), 2.18-2.16 (m, |
| | | | 1H), 1.67 (d, J = 12.00 Hz, 2H), 1.56-1.48 (m, 1H), 1.34- |
| | | | 1.25 (m, 1H), 1.09-0.96 (m, 2H), 0.70-0.68 (m, 1H), 0.51 |
| | | | (t, J = 11.20 Hz, 2H), 0.35-0.34 (m, 1H). |
| 263 | 545a | m/z = | ¹H NMR (400 MHz, DMSO-d6): 1H NMR (400 MHz, |
| | | 814.4 | DMSO-d6) δ 10.84 (s, 1H), 8.88 (s, 1H), 8.19 (s, 1H), 8.07 |
| | | [M + H]⁺ | (s, 1H), 7.73 (d, J = 9.0 Hz, 1H), 7.48 (dd, J = 12.4, 9.0 Hz, |
| | | | 2H), 6.92 (d, J = 9.1 Hz, 1H), 6.87 (s, 1H), 6.18 (s, 1H), |
| | | | 4.56-4.29 (m, 3H), 4.26 (dd, J = 9.2, 5.2 Hz, 1H), 3.89 (s, |
| | | | 3H), 3.73 (d, J = 11.7 Hz, 2H), 3.57 (s, 3H), 3.26-3.23 |
| | | | (m, 2H), 2.94 (t, J = 12.5 Hz, 1H), 2.89-2.74 (m, 3H), |
| | | | 2.70-2.54 (m, 3H), 2.48-2.41 (m, 1H), 2.36-2.24 (m, |
| | | | 2H), 2.21-2.05 (m, 3H), 1.36-1.25 (m, 1H), 0.75-0.65 |
| | | | (m, 1H), 0.59-0.45 (m, 2H), 0.42-0.29 (m, 1H). |
| 264 | 572a | m/z = | ¹H NMR (400 MHz, DMSO-d6): δ 10.84 (s, 1H), 8.73 (s, |
| | | 886.5 | 1H), 8.18-8.11 (m, 1H), 8.02-7.97 (m, 1H), 7.77-7.70 |
| | | [M + H]⁺ | (m, 1H), 7.53-7.40 (m, 2H), 6.94-6.87 (m, 1H), 6.83 (s, |
| | | | 1H), 6.29-6.12 (m, 1H), 4.57-4.28 (m, 2H), 4.28-4.20 |
| | | | (m, 1H), 3.88 (s, 3H), 3.56 (s, 3H), 3.31 (s, 8H), 3.22- |
| | | | 3.17 (m, 6H), 2.86-2.70 (m, 2H), 2.70-2.56 (m, 2H), |
| | | | 2.38-2.10 (m, 2H), 1.98-1.84 (m, 1H), 1.48-1.27 (m, |
| | | | 4H), 0.76-0.62 (m, 1H), 0.59-0.45 (m, 2H), 0.41-0.25 |
| | | | (m, 1H). |
| 265 | 573a | m/z = | ¹H NMR (400 MHz, DMSO-d6): δ 10.84 (s, 1H), 8.75 (s, |
| | | 854.5 | 1H), 8.22 (s, 1H), 8.05 (s, 1H), 7.83 (s, 1H), 7.50 (d, J = 8.6 |
| | | [M + H]⁺ | Hz, 1H), 7.43 (d, J = 9.1 Hz, 1H), 6.95-6.89 (m, 1H), 6.84 |
| | | | (s, 1H), 6.16 (s, 1H), 4.51-4.20 (m, 3H), 3.89 (s, 3H), |
| | | | 3.55 (s, 3H), 3.29-2.96 (m, 7H), 2.72-2.54 (m, 2H), 2.36- |
| | | | 2.10 (m, 2H), 2.08 (s, 2H), 1.39-1.27 (m, 1H), 0.89- |
| | | | 0.63 (m, 3H), 0.61-0.42 (m, 2H), 0.41-0.27 (m, 1H). |
| 266 | 534d | m/z = | ¹H NMR (400 MHz, DMSO-d6): δ = 10.82 (s, 1H), 8.80 |
| | | 881.3 | (s, 1H), 8.42 (s, 1H), 8.28-8.10 (m, 1H), 8.04 (s, 1H), 7.83 |
| | | [M + H]⁺ | (br d, J = 8.6 Hz, 1H), 7.43 (d, J = 9.3 Hz, 1H), 7.36-7.26 |
| | | | (m, 1H), 6.50-6.40 (m, 2H), 6.19 (br s, 1H), 5.76-5.56 |
| | | | (m, 1H), 4.53-4.30 (m, 2H), 4.17 (dd, J = 5.1, 8.8 Hz, 1H), |
| | | | 3.81 (s, 3H), 3.74-3.60 (m, 4H), 3.57 (s, 3H), 3.29-3.14 |
| | | | (m, 2H), 2.69-2.55 (m, 2H), 2.46-2.35 (m, 1H), 2.33- |
| | | | 2.08 (m, 3H), 2.05-1.95 (m, 1H), 1.88 (br d, J = 13.3 Hz, |
| | | | 1H), 1.71-1.55 (m, 2H), 1.39-1.18 (m, 2H), 0.79-0.66 |
| | | | (m, 1H), 0.52 (br t, J = 5.8 Hz, 2H), 0.40-0.27 (m, 1H). |
| 267 | 534c | m/z = | ¹H NMR (400 MHz, DMSO-d6): δ = 10.82 (s, 1H), 8.78 |
| | | 441.6 | (s, 1H), 8.28-8.08 (m, 1H), 8.03 (s, 1H), 7.92-7.76 (m, |
| | | [M + H]⁺ | 1H), 7.43 (d, J = 9.2 Hz, 1H), 7.34 (d, J = 8.8 Hz, 1H), 6.67 |
| | | | (d, J = 8.8 Hz, 1H), 6.48 (s, 1H), 6.22-6.07 (m, 1H), 5.79 |
| | | | (t, J = 2.8, 4.0 Hz,1H), 4.56-4.25 (m, 2H), 4.18 (dd, J = |
| | | | 5.2, 8.8 Hz, 1H), 4.12-4.04 (m, 1H), 3.81 (s, 3H), 3.78- |
| | | | 3.62 (m, 4H), 3.57 (s, 3H), 2.60 (d, J = 5.6 Hz, 4H), 2.31- |
| | | | 2.11 (m, 3H), 1.95- 1.72 (m, 3H), 1.71-1.58 (m, 1H), 1.54- |
| | | | 1.36 (m, 1H), 1.33 (dd, J = 3.2, 5.6 Hz, 1H), 0.77-0.66 |
| | | | (m, 1H), 0.58-0.45 (m, 2H), 0.41-0.28 (m, 1H). |
| 268 | 417a | m/z = | ¹H NMR (400 MHz, DMSO-d6): δ 10.82 (s, 1H), 8.82 (d, J = |
| | | 797.44 | 4.40 Hz, 1H), 8.18 (t, J = 2.00 Hz, 1H), 8.05 (s, 1H), 7.73 |
| | | [M − H]⁻ | (d, J = 8.40 Hz, 1H), 7.46-7.42 (m, 2H), 6.49 (d, J = 8.80 |
| | | | Hz, 1H), 6.28-6.16 (m, 2H), 4.46-4.41 (m, 2H), 4.21-4.20 |
| | | | (m, 1H), 3.95-3.81 (s, 5H), 3.60 (s, 4H), 3.41-3.32 (m, 5H), |
| | | | 3.07-3.06 (m , 1H), 2.61-2.58 (m, 4H), 2.24-2.16 (m, 2H), |
| | | | 1.76-1.74 (m, 2H), 1.36-1.32 (m, 2H), 0.47-0.44 (m, 4H). |
| 269 | 511b | m/z = | ¹H NMR (400 MHz, DMSO-d6): δ 10.84 (s, 1H), 8.75 (s |
| | | 885.40 | 1H), 8.19 (bs, 1H), 8.02 (s, 1H), 7.75 (d, J = 8.8 Hz, 1H), |
| | | [M + H]⁺ | 7.48-7.43 (m, 2H), 6.89 (d, J = 9.2 Hz, 1H), 6.80 (bs, 1H), |
| | | | 6.17 (bs, 1H), 4.58-4.38 (m, 4H), 4.26-4.22 (m, 1H), 3.88- |
| | | | 3.85 (m, 6H), 3.56 (s, 3H), 3.35 (bs, 1H), 2.69-2.64 (m1H), |
| | | | 2.62-2.49 (m, 5H), 2.33-2.28 (m, 1H), 2.17-2.16 (m, 1H), |
| | | | 1.63-1.60 (m, 2H), 1.57-1.47 (m, 6H), 1.35-1.23 (m, 3H), |
| | | | 0.91 (s, 3H), 0.70 (bs, 1H), 0.53-0.50 (m, 2H), 0.36-0.35 |
| | | | (m, 1H). |
| 270 | 499b | m/z = | ¹H NMR (400 MHz, DMSO-d6): δ 10.89 (s, 1H), 9.01 (br |
| | | 870.42 | s, 1H), 8.90 (s, 1H), 8.19 (s, 1H), 8.06 (s, 1H), 7.72-7.70 |
| | | [M + H]⁺ | (d, J = 9.2 Hz, 1H), 7.50 (s, J = 2 Hz, 1H), 7.43 (d, J = 9.2 |
| | | | Hz, 1H), 7.09-7.00 (m, 2H), 6.22 (s, 1H), 4.56-4.34 (m, |
| | | | 5H), 4.25 (s, 3H), 3.50-3.40 (m, 3H), 3.30-3.10 (m, 6H), |
| | | | 2.80 (t, J = 2 Hz, 2H), 2.70-2.50 (m, 2H), 2.20 (s, 3H), |
| | | | 1.68-1.50 (m, 2H), 1.40 (s, 4H), 1.32-1.20 (m, 5H), 0.71- |
| | | | 0.62 (m, 1H), 0.60-0.45 (m, 2H), 0.40-0.32 (m, 1H) |

### Example P1

### Resolution of Stereoisomeric Mixtures

Mixtures of stereoisomers were resolved using methods described in Table P1.

| | | Retention | |
|---|---|---|---|
| Starting Material | Separation Method | Time | Final Product |
| tert-butyl 4-(1-(1-(3-(2,6- | Column: Chiralpak IH (250 × | R_{T} = 5.79 min | 379a |
| bis(benzyloxy)pyridin-3-yl)- | 4.6 mm, internal diameter: 5 μm); | | (Example 10) |
| 1-methyl-1H-indazol-6- | mobile phase: Phase A (CO₂), and | | |
| yl)piperidin-4- | Phase B (iPrOH with 0.2% | R_{T} = 6.44 min | 379b |
| yl)ethyl)piperazine-1- | isopropylamine); isocratic elution | | (Example 12) |
| carboxylate | B %: 30%; flow rate: 3 mL/min; | | |
| | detector: PDA; wavelength: 230 nm; | | |
| | column temperature: 35° C.; back | | |
| | pressure: 100 bar | | |
| tert-butyl 4-(1-(1- | Column: Chiralcel OX-H (250 × | R_{T} = 6.67 min | 380a |
| ((benzyloxy)carbonyl) | 4.6mm, internal diameter: 5 μm); | | (Example 7) |
| piperidin-4-yl)ethyl) | mobile phase: Phase A (CO₂), and | | |
| piperazine-1-carboxylate | Phase B (MeOH with 0.5% | | |
| | diethylamine); isocratic elution B %: | | |
| | 20%; flow rate: 3 mL/min; detector: | R_{T} = 5.77 min | 380b |
| | PDA; wavelength: 220 nm; column | | (Example 9) |
| | temperature: 30° C.; back pressure: | | |
| | 100 bar | | |
| tert-butyl 7-((3-(2,6- | Column: Chiralpak IH (30 × 250 mm, | R_{T} = 10.1 min | 464a |
| bis(benzyloxy)pyridin-3-yl)- | internal diameter: 5 μm); mobile | | (Example 103) |
| 1-methyl-1H-indazol-6- | phase: Phase A (CO₂), and Phase B | | |
| yl)amino)-6-methyl-2- | (MeOH with 0.2% diethylamine); | R_{T} = 8.13 min | 464b |
| azaspiro[3.5]nonane-2- | isocratic elution B %: 50%; flow rate: | | (Example 105) |
| carboxylate | 100 mL/min; detector: PDA; | R_{T} = 7.36 min | 464c |
| | wavelength: 225 nm; column | | (Example 110) |
| | temperature: 35° C.; back pressure: | R_{T} = 10.7 min | 464d |
| | 100 bar | | (Example 127) |
| tert-butyl 7-((3-(2,6- | Column: Lux-Cellulose-4 (30 × | R_{T} = 4.24 min | 465a |
| bis(benzyloxy)pyridin-3-y1)- | 250 mm, internal diameter: 5 μm); | | (Example 104) |
| 1-methyl-1H-indazol-7- | mobile phase: Phase A (CO₂), and | | |
| yl)amino)-6-methyl-2- | Phase B (MeCN/MeOH with 0.2% | R_{T} = 3.19 min | 465b |
| azaspiro[3.5]nonane-2- | NH₃•H₂O); isocratic elution B %: | | (Example 109) |
| carboxylate | 40%; flow rate: 100 mL/min; | R_{T} = 6.09 min | 465c |
| | detector: PDA; wavelength: 225 nm; | | (Example 111) |
| | column temperature: 30° C.; back | R_{T} = 3.07 min | 465d |
| | pressure: 100 bar | | (Example 112) |
| tert-butyl 7-hydroxy-6- | Column: Chiralpak AD-3 (50 mm× | R_{T} = 1.28 min | 492a |
| methyl-2- | 4.6 mm, 3 μm); mobile phase: Phase | | (Example 121) |
| azaspiro[3.5]nonane-2- | A (CO₂), and Phase B (iPrOH with | | |
| carboxylate | 0.05% diethylamine); Gradient | R_{T} = 1.16 min | 492b |
| | elution: B in A from 5% to 40%; flow | | (Example 245) |
| | rate: 3 mL/min; detector: PDA; | R_{T} = 1.27 min | 492c |
| | wavelength: 220 nm; column | | (Example 246) |
| | temperature: 35° C.; back pressure: | R_{T} = 1.04 min | 492d |
| | 100 bar | | (Example 247) |
| benzyl 4-(1-(1-(tert- | Column: Chiralpak AD-H (250 mm × | R_{T} = 3.84 min | 499a |
| butoxycarbonyl)piperidin-4- | 4.6 mm, 5 μm); mobile phase: Phase | | (Example 153) |
| yl)ethyl)piperazine-1- | A (CO₂), and Phase B (MeOH); | | 523a |
| carboxylate | isocratic elution B %: 25%, flow rate: | | (Example 162) |
| | 3 mL/min; detector: PDA; | R_{T} = 3.79 min | 499b |
| | wavelength: 208 nm; column | | (Example 270) |
| | temperature: 30° C.; back pressure: | | 523b |
| | 100 bar | | (Example 163) |
| benzyl 4-(1-(1-(3-(2,6- | Column: Lux-Cellulose-4 (4.6 × | R_{T} = 5.37 min | 500a |
| bis(benzyloxy)pyridin-3-yl)- | 150 mm, internal diameter: 3 μm); | | (Example 184) |
| 1-methyl-1H-indazol-7- | mobile phase: Phase A (CO₂), and | | |
| yl)piperidin-4-yl)-1- | Phase B (MeCN/MeOH with 0.2% | | |
| hydroxyethyl)piperidine-1- | NH₃•H₂O); isocratic elution B %: | | |
| carboxylate | 50%; flow rate: 3 mL/min; detector: | R_{T} = 6.47 min | 500b |
| | PDA; wavelength: 230 nm; column | | (Example 154) |
| | temperature: 30° C.; back pressure: | | |
| | 100 bar | | |
| tert-butyl 4-(1-(4-(3-(2,6- | Column: Chiralpak IJ (250 mm × 4.6 | R_{T} = 3.75 min | 522a |
| bis(benzyloxy)pyridin-3-y1)- | mm, 5 μm); mobile phase: Phase A | | (Example 160) |
| 1-methyl-1H-indazol-6-yl)- | (CO₂), and Phase B (MeCN/MeOH | | |
| 3,6-dihydropyridin-1(2H)- | with 0.2% NH₃•H₂O); isocratic | | |
| yl)ethyl)piperidine-1- | elution B %: 40%; flow rate: | | |
| carboxylate | 3 mL/min; detector: PDA; | R_{T} = 4.62 min | 522b |
| | wavelength: 258 nm; column | | (Example 161) |
| | temperature: 30° C.; back pressure: | | |
| | 100 bar | | |
| tert-butyl 4-((3-((3-(2- | Column: Chiralpak AD-H (250 mm × | R_{T} = 8.48 min | 575a |
| (benzyloxy)-6- | 4.6 mm, 5 μm); mobile phase: Phase | | (Example 248) |
| hydroxypyridin-3-yl)-1- | A (CO₂), and Phase B (iPrOH with | | |
| methyl-1H-indazol-6- | 0.2% isopropylamine); isocratic | | |
| yl)oxy)pyrrolidin-1- | elution B %: 50%; flow rate: | | |
| yl)methyl)piperidine-1- | 3 mL/min; detector: PDA; | R_{T} = 11.18 min | 575b |
| carboxylate | wavelength: 321 nm; column | | (Example 249) |
| | temperature: 35° C.; back pressure: | | |
| | 100 bar | | |
| tert-butyl 4-((4-(3-(2- | Column: Chiralpak IJ (250 mm × 4.6 | R_{T} = 3.12 min | 582a |
| (benzyloxy)-6- | mm, 5 μm); mobile phase: Phase A | | (Example 255) |
| hydroxypyridin-3-y1)-1- | (CO₂), and Phase B (MeCN/MeOH | | |
| methyl-1H-indazol-7- | with 0.2% NH₃•H₂O); isocratic | | |
| yl)piperazin-1-yl)methyl)- | elution B %: 30%; flow rate: | | |
| 3,3-dimethylpiperidine-1- | 3 mL/min; detector: PDA; | R_{T} = 4.11 min | 582b |
| carboxylate | wavelength: 330 nm; column | | (Example 256) |
| | temperature: 30° C.; back pressure: | | |
| | 100 bar | | |
| Trans-tert-butyl-4-((4-(3-(2- | Column: Chiralpak IJ (250 mm × 4.6 | R_{T} = 12.04 min | 578a |
| (benzyloxy)-6- | mm, 5 μm); mobile phase: Phase A | | (Example 251) |
| hydroxypyridin-3-yl)-1- | (CO₂), and Phase B (iPrOH with | | |
| methyl-1H-indazol-6- | 0.2% isopropylamine); isocratic | | |
| yl)piperazin-1-yl)methyl)-3- | elution B %: 50%; flow rate: | | |
| fluoropiperidine-1- | 3 mL/min; detector: PDA; | R_{T} = 12.31 min | 578b |
| carboxylate | wavelength: 260 nm; column | | (Example 252) |
| | temperature: 35° C.; back pressure: | | |
| | 100 bar | | |
| Trans-tert-butyl-4-((4-(3-(2- | Column: Lux-Cellulose-4 (4.6 × | R_{T} = 10.20 min | 579a |
| (benzyloxy)-6- | 150 mm, internal diameter: 3 μm); | | (Example 254) |
| hydroxypyridin-3-yl)-1- | mobile phase: Phase A (CO₂), and | | |
| methyl-1H-indazol-7- | Phase B (MeCN/MeOH with 0.2% | | |
| yl)piperazin-1-yl)methyl)-3- | NH₃•H₂O); isocratic elution B %: | R_{T} = 8.23 min | 579b |
| fluoropiperidine-1- | 40%; flow rate: 3 mL/min; detector: | | (Example 253) |
| carboxylate | PDA; wavelength: 330 nm; column | | |
| | temperature: 30° C.; back pressure: | | |
| | 100 bar | | |

### Example B1

### HiBiT Based Degradation Assay

Human BCL6 protein coding open reading frame fused with N-terminal HiBiT coding sequence was synthesized from Integrated DNA Technologies (IDT). Next, the N-HiBiT-BCL6 sequence was cloned into pLV-UBC-PGK-Puro, a lentivirus plasmid purchased from Vectorbuilder, to generate pLV-UBC-N-HiBiT-BCL6-PGK-puro. Lentiviral particles were generated from Lenti-X™ 293T cells (Clontech) by co-transfection of pLV-UBC-N-HiBiT-BCL6-PGK-puro plasmids and lentiviral packaging plasmid mix (Cellecta). HT1080 [HT1080](ATCC CCL-121) cells were infected with the lentivirus, and HT1080 cells stably integrated with the lentiviral vectors were established by incubation with 1 g/mL puromycin (Thermofisher).

HT1080 cells expressing N-terminal HiBiT tagged BCL6 were dispensed into a 384-well plate pre-spotted with compounds at varying concentrations. Five thousand cells were seeded into each well in 40 μL of RPMI 1640 media plus 10% Fetal Bovine Serum (10082-147, Thermofisher). After 5 hours of incubation at 37° C. with 5% CO₂, 30 μL of the NANO-GLO® HiBiT Lytic Detection System working solution (Promega) was added to each well and incubated at room temperature for 15 min. After incubation, luminescence was read on a PHERAstar FSX Plate Reader (BMG). BCL6 degradation at each indicated concentration was normalized with DMSO control. The BCL6 degradation curves were plotted using a four-parameter logistic model.

Table B1 provides the Yₘᵢₙ and EC₅₀ values for certain compounds of this disclosure.

TABLE B1

| Compound | Yₘᵢₙ | EC₅₀ |
|---|---|---|
| No. | (5 hr) | (5 hr) |
| 101a | +++ | +++ |
| 102a | ++ | +++ |
| 130a | +++ | +++ |
| 132a | +++ | +++ |
| 133a | +++ | +++ |
| 170a | + | ++ |
| 186a | + | +++ |
| 194a | +++ | +++ |
| 214a | ++ | +++ |
| 214b | +++ | +++ |
| 374a | +++ | +++ |
| 375a | ++ | ++ |
| 379a | ++ | +++ |
| 379b | ++ | +++ |
| 380a | +++ | +++ |
| 380b | ++ | ++ |
| 381a | ++ | +++ |
| 381b | ++ | +++ |
| 382a | ++ | +++ |
| 383a | +++ | +++ |
| 384a | +++ | +++ |
| 385a | +++ | +++ |
| 386a | +++ | +++ |
| 387a | ++ | +++ |
| 388a | ++ | +++ |
| 389a | ++ | +++ |
| 390a | ++ | +++ |
| 391a | ++ | +++ |
| 392a | ++ | +++ |
| 393a | ++ | +++ |
| 394a | ++ | +++ |
| 395a | ++ | +++ |
| 396a | + | +++ |
| 397a | +++ | +++ |
| 398a | ++ | +++ |
| 399a | + | ++ |
| 400a | +++ | +++ |
| 401a | + | +++ |
| 402a | + | +++ |
| 403a | ++ | +++ |
| 404a | ++ | +++ |
| 405a | ++ | +++ |
| 406a | ++ | +++ |
| 407a | ++ | +++ |
| 408a | ++ | +++ |
| 409a | + | +++ |
| 410a | ++ | +++ |
| 411a | +++ | +++ |
| 412a | +++ | +++ |
| 413a | ++ | +++ |
| 414a | ++ | +++ |
| 415a | ++ | +++ |
| 416a | +++ | +++ |
| 417a | + | +++ |
| 418a | ++ | +++ |
| 419a | +++ | +++ |
| 420a | +++ | +++ |
| 421a | +++ | +++ |
| 422a | ++ | +++ |
| 423a | + | +++ |
| 424a | + | +++ |
| 425a | +++ | +++ |
| 426a | ++ | ++ |
| 426b | ++ | ++ |
| 427a | ++ | +++ |
| 428a | ++ | +++ |
| 429a | ++ | +++ |
| 430a | ++ | +++ |
| 431a | +++ | +++ |
| 432a | +++ | +++ |
| 433a | ++ | ++ |
| 434a | ++ | +++ |
| 435a | +++ | +++ |
| 436a | ++ | +++ |
| 437a | ++ | +++ |
| 438a | +++ | +++ |
| 439a | ++ | +++ |
| 440a | ++ | +++ |
| 441a | ++ | +++ |
| 442a | +++ | +++ |
| 442b | +++ | +++ |
| 443a | +++ | +++ |
| 443b | ++ | +++ |
| 444a | +++ | +++ |
| 445a | ++ | +++ |
| 446a | ++ | +++ |
| 447a | ++ | +++ |
| 448a | ++ | +++ |
| 449a | +++ | +++ |
| 450a | ++ | +++ |
| 451a | +++ | +++ |
| 451b | +++ | +++ |
| 452a | +++ | +++ |
| 453a | +++ | +++ |
| 454a | ++ | +++ |
| 455a | ++ | +++ |
| 456a | +++ | +++ |
| 457a | ++ | +++ |
| 458a | +++ | +++ |
| 459a | ++ | +++ |
| 459b | ++ | +++ |
| 460a | ++ | +++ |
| 460b | +++ | +++ |
| 461a | +++ | +++ |
| 462a | +++ | +++ |
| 463a | ++ | +++ |
| 464a | +++ | +++ |
| 464b | ++ | +++ |
| 464c | +++ | +++ |
| 464d | +++ | +++ |
| 465a | ++ | ++ |
| 465b | ++ | +++ |
| 465c | + | +++ |
| 465d | + | +++ |
| 466a | ++ | +++ |
| 466b | +++ | +++ |
| 467a | +++ | +++ |
| 468a | ++ | +++ |
| 469a | +++ | +++ |
| 470a | ++ | +++ |
| 471a | ++ | +++ |
| 472a | +++ | +++ |
| 473a | +++ | +++ |
| 474a | +++ | +++ |
| 475a | +++ | +++ |
| 475b | +++ | +++ |
| 476a | ++ | +++ |
| 476b | ++ | +++ |
| 477a | +++ | +++ |
| 478a | ++ | +++ |
| 479a | +++ | +++ |
| 480a | ++ | +++ |
| 481a | ++ | +++ |
| 481b | ++ | +++ |
| 482a | ++ | +++ |
| 482b | +++ | +++ |
| 483a | ++ | ++ |
| 484a | +++ | +++ |
| 485a | +++ | +++ |
| 486a | +++ | +++ |
| 486b | ++ | +++ |
| 486c | ++ | +++ |
| 486d | ++ | +++ |
| 487a | +++ | +++ |
| 487b | +++ | +++ |
| 487c | ++ | +++ |
| 487d | +++ | +++ |
| 488a | ++ | +++ |
| 489a | +++ | +++ |
| 490a | + | +++ |
| 491a | ++ | ++ |
| 491b | ++ | +++ |
| 492a | +++ | +++ |
| 492b | +++ | +++ |
| 492c | +++ | +++ |
| 492d | +++ | +++ |
| 493a | +++ | +++ |
| 493b | +++ | +++ |
| 494a | +++ | +++ |
| 495a | +++ | +++ |
| 496a | +++ | ++ |
| 496b | +++ | +++ |
| 497a | ++ | +++ |
| 498a | ++ | ++ |
| 499a | +++ | +++ |
| 499b | +++ | +++ |
| 500a | ++ | +++ |
| 500b | ++ | +++ |
| 501a | +++ | +++ |
| 502a | + | +++ |
| 503a | ++ | +++ |
| 504a | +++ | +++ |
| 505a | +++ | +++ |
| 506a | ++ | +++ |
| 506b | +++ | +++ |
| 507a | +++ | +++ |
| 508a | ++ | +++ |
| 509a | ++ | +++ |
| 510a | +++ | +++ |
| 511a | ++ | +++ |
| 511b | ++ | +++ |
| 512a | ++ | +++ |
| 512b | ++ | +++ |
| 513a | +++ | +++ |
| 514a | +++ | +++ |
| 516a | +++ | +++ |
| 518a | +++ | +++ |
| 519a | +++ | +++ |
| 520a | +++ | +++ |
| 521a | ++ | +++ |
| 522a | +++ | +++ |
| 522b | +++ | +++ |
| 523a | +++ | +++ |
| 523b | ++ | +++ |
| 524a | ++ | +++ |
| 525a | +++ | +++ |
| 525b | +++ | +++ |
| 525c | +++ | +++ |
| 525d | +++ | +++ |
| 526a | +++ | +++ |
| 527a | +++ | +++ |
| 527b | +++ | +++ |
| 528a | +++ | +++ |
| 529a | ++ | ++ |
| 529b | ++ | +++ |
| 529c | ++ | +++ |
| 530a | ++ | +++ |
| 531a | +++ | +++ |
| 532a | +++ | +++ |
| 533a | +++ | +++ |
| 534a | +++ | +++ |
| 534b | +++ | +++ |
| 535a | ++ | +++ |
| 536a | + | +++ |
| 537a | +++ | +++ |
| 538a | +++ | +++ |
| 538b | +++ | +++ |
| 538c | +++ | +++ |
| 539a | ++ | +++ |
| 540a | +++ | +++ |
| 541a | ++ | +++ |
| 542a | ++ | +++ |
| 543a | + | ++ |
| 544a | ++ | +++ |
| 545a | + | +++ |
| 546a | + | +++ |
| 547a | +++ | +++ |
| 548a | ++ | +++ |
| 548b | ++ | +++ |
| 549a | +++ | +++ |
| 550a | +++ | +++ |
| 551a | ++ | ++ |
| 552a | +++ | +++ |
| 553a | +++ | +++ |
| 554a | ++ | +++ |
| 555a | + | +++ |
| 556a | + | +++ |
| 557a | ++ | +++ |
| 558a | ++ | +++ |
| 559a | +++ | +++ |
| 559b | +++ | +++ |
| 560a | + | +++ |
| 560b | ++ | +++ |
| 561a | +++ | +++ |
| 562a | ++ | +++ |
| 563a | +++ | +++ |
| 563b | ++ | +++ |
| 564a | +++ | +++ |
| 565a | ++ | +++ |
| 566a | ++ | +++ |
| 568a | ++ | +++ |
| 568b | +++ | +++ |
| 569a | + | +++ |
| 570a | ++ | +++ |
| 572a | ++ | +++ |
| 573a | +++ | +++ |
| 574a | +++ | +++ |
| 575a | ++ | +++ |
| 575b | +++ | +++ |
| 576a | +++ | +++ |
| 577a | +++ | +++ |
| 578a | +++ | +++ |
| 578b | +++ | +++ |
| 579a | +++ | +++ |
| 579b | +++ | +++ |
| 581a | +++ | +++ |
| 582a | +++ | +++ |
| 582b | +++ | +++ |

Notes:
Yₘᵢₙ ≤ 30%: “+++”; 30% < Yₘᵢₙ ≤ 50%: “++”; 50% < Yₘᵢₙ ≤ 70%; “+”.
EC₅₀ ≤ 25 nM: “+++”; 25 nM < EC₅₀ ≤ 100 nM: “++”; 100 nM < EC₅₀ ≤ 1000 nM: “+”.

### Example B2

### Cell Proliferation Assay

OCI-Ly1 (DSMZ: ACC 722), SU-DHL-5 (ATCC CRL-2958), Toledo (ATCC CRL-2631), and WSU-DLCL2 (DSMZ: ACC 575) cells are seeded at 1,000 cells per well in 50 μL RPMI 1640 media plus 10% Fetal Bovine Serum (10082-147, Thermofisher) in black 384-well plates. The plates are pre-spotted with compounds at varying concentrations. After 5 days of incubation at 37° C. with 5% CO₂, cell viabilities are assessed using CELLTITER-GLO® Cell Viability Assay kit according to manufacturer's instructions (Promega). Relative cell proliferation at each concentration is normalized against DMSO control.

### REFERENCES

### OCI-Ly1 Cells:

1) Chang, H., Blondal, et al. (1995). Leukemia & Lymphoma 19 (1-2): 165-171.
2) Tweeddale, M. E., eta 1. (1987). Blood 69 (5): 1307-1314.
3) Farrugia, M. M., et al. (1994). Blood 83 (1): 191-198.
4) Mehra, S., et al. (2002). Genes Chromosomes Cancer 33 (3): 225-234.
5) Kuppers, R., et al. (2003). Journal of Clinical Investigation 111 (4): 529-537.
6) Ngo, V. N., et al. (2011). Nature 470 (7332): 115-9.

### WSU-DLCL2 Cells:

1) Al-Katib, A. M., et al. (1998). Clinical Cancer Research 4 (5): 1305-1314.
2) Mohammad, R. M., et al. (2000). Clinical Cancer Research 6 (12): 4950-4956.
3) Morin, R. D., et al. (2010). Nature Genetics 42 (2): 181-185.
4) Quentmeier, H., et al. (2019). Scientific Reports 9 (1): 8218.

### Example B3

### Pharmacokinetic Assessment

Pharmacokinetics (PK) studies are conducted on male CD-1 mice by two delivery routes: intravenous (IV) injection and oral gavage (PO). Mice for the IV group (n=3) are allowed free access to food and water and mice for the PO group (n=3) are fasted 6-8 hours prior to dosing. The test articles are formulated in solution for the IV route (commonly 5% DMSO/10% Solutol HS15/85% water (pH 5)) and solution or suspension for the PO route (commonly 100% PEG400). On the day of the experiment, test article is administered via vein injection (commonly 1 mg/kg) for IV route or via oral gavage (commonly 10 mg/kg) for PO route, respectively. Blood samples are collected via the saphenous vein using a needle (commonly 20G) at 0.83 to 24 hours post dose. Approximately 30 μL of blood per timepoint is collected into pre-chilled tubes using K₂EDTA as the anti-coagulant. After collection of blood samples at each time point, the blood samples are stored on ice. Blood samples are centrifuged within 0.5 hour of collection to separate plasma. Centrifugation is conducted at 2500×g for 15 minutes at 4° C. Plasma samples are immediately acidified (commonly 0.1M sodium citrate (pH=5) buffer). The samples are then submitted to LC-MS/MS for sample analysis. Pharmacokinetics parameters, including clearance (IV), area under the curve (AUC), and oral bioavailability (% F) are calculated by non-compartmental model.

### EXEMPLARY EMBODIMENTS

### P01 Embodiments

Embodiment 1. A compound of Formula (I):

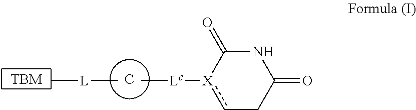

or a pharmaceutically acceptable salt thereof, wherein:
TBM is (T1):

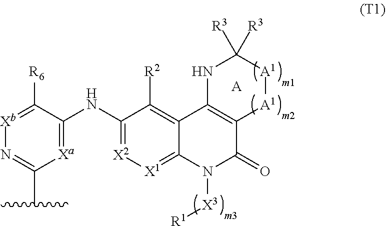

X¹ is selected from the group consisting of N and CR²;
X² is CH;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
m3 is 0 or 1;
X³ is C₁₋₃ alkylene optionally substituted with 1-3 R^{c};
R¹ is selected from the group consisting of: H and C₃₋₆ cycloalkyl;
m1 is 2; each A¹ is independently CH₂, CHR⁴, or CR⁴R⁴;
m2 is 1; A² is —O—;
one R³ is selected from the group consisting of:
(i) C₃₋₆ cycloalkyl optionally substituted with 1-3 R⁹, and
(ii) C₁₋₃ alkyl optionally substituted with 1-3 —F; and the other R³ is H;
each R⁴ is independently selected from the group consisting of: H, Ra, and R^{b};
X^{a} is selected from the group consisting of: N, CH, and CF;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1} L^{A3}, and L^{A4} provided that 1-3 occurrences of L^{A} is L^{A4};

### (BB)

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, N—N, N—S(O)o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{e};
Ring C is selected from the group consisting of:

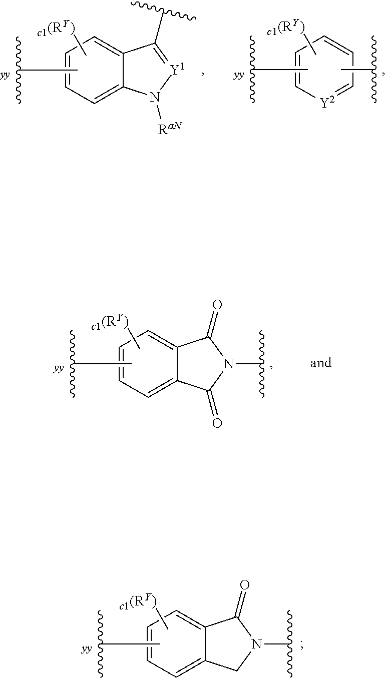

c1 is 0, 1, 2, or 3;
each R^{Y} is independently selected from the group consisting of: R^{a} and R^{b};
R^{aN} is H or C₁₋₆ alkyl optionally substituted with 1-3 R^{e};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)C₁₋₆ haloalkyl;
(i) C(═O)OH;
(j) C(═O)OC₁₋₆ alkyl;
(k) C(═O)OC₁₋₆ haloalkyl;
(l) C(═O)N(R^{f})₂;
(m) S(O)₀₋₂(C₁₋₆ alkyl);
(n) S(O)₀₋₂(C₁₋₆ haloalkyl);
(o) S(O)₁₋₂N(R^{f})₂; and
(p) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{e} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

Embodiment 2. The compound of Embodiment 1, wherein Ring C is

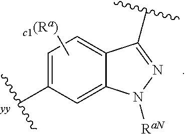

Embodiment 3. The compound of Embodiment 1, wherein Ring C is

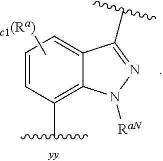

Embodiment 4. The compound of Embodiment 1, wherein Ring C is

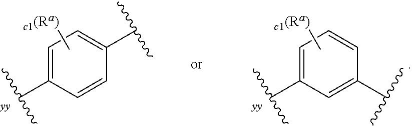

Embodiment 5. The compound of Embodiment 1, wherein Ring C is

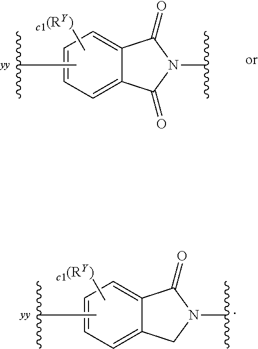

Embodiment 6. The compound of any one of Embodiments 1-5, wherein c1 is 0.

Embodiment 7. The compound of any one of Embodiments 1-5, wherein c1 is 1; and R^{Y} is halo (e.g., —F).

Embodiment 8. The compound of any one of Embodiments 1-3, wherein R^{aN} is C₁₋₃ alkyl (e.g., methyl).

Embodiment 9. The compound of any one of Embodiments 1-8, wherein X is CH.

Embodiment 10. The compound of any one of Embodiments 1-9, wherein L^{C} is a bond.

Embodiment 11. The compound of Embodiment 1, wherein the

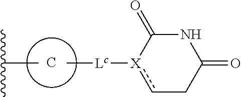

moiety is selected from the group consisting of the moieties delineated in Table CM-1a:

TABLE CM-1

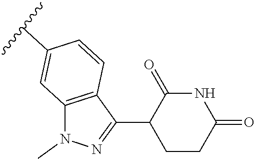 [SMILES: *c1ccc2c(C3CCC(=O)NC3=O)nn(C)c2c1]

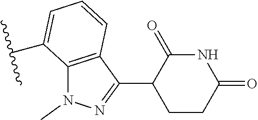 [SMILES: *c1cccc2c(C3CCC(=O)NC3=O)nn(C)c12]

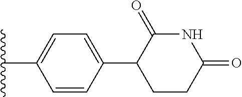 [SMILES: *c1ccc(C2CCC(=O)NC2=O)cc1]

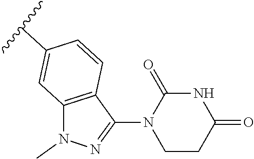 [SMILES: *c1ccc2c(N3CCC(=O)NC3=O)nn(C)c2c1]

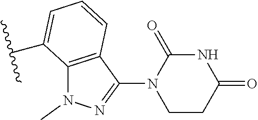 [SMILES: *c1cccc2c(N3CCC(=O)NC3=O)nn(C)c12]

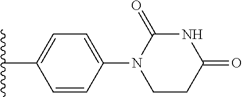 [SMILES: *c1ccc(N2CCC(=O)NC2=O)cc1]

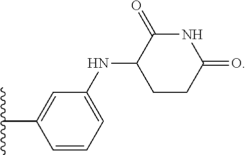 [SMILES: *c1cccc(NC2CCC(=O)NC2=O)c1]

Embodiment 12. The compound of Embodiment 11, wherein

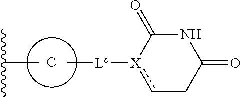

moiety is

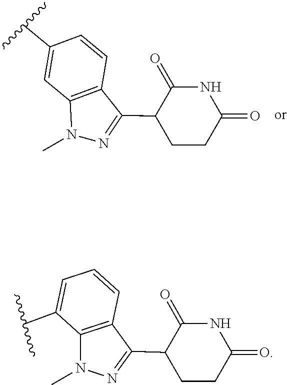 [SMILES: *c1ccc2c(C3CCC(=O)NC3=O)nn(C)c2c1 ; "or" ; *c1cccc2c(C3CCC(=O)NC3=O)nn(C)c12]

Embodiment 13. The compound of any one of Embodiments 1-12, wherein -A¹)ₘ₁ is —C(R⁴R⁴)—CH₂—* (e.g., —CF₂—CH₂—*), wherein * represents the point of attachment to -(A²)ₘ₂-.

Embodiment 14. The compound of any one of Embodiments 1-13, wherein one R³ is C₃₋₆ cycloalkyl; and the other R³ is H.

Embodiment 15. The compound of any one of Embodiments 1-14, wherein one R³ is cyclopropyl; and the other R³ is H.

Embodiment 16. The compound of any one of Embodiments 1-15, wherein one R³ is cyclopropyl; the other R³ is H; and -(A¹)ₘ₁- is —CF₂—CH₂—*, wherein * represents the point of attachment to -(A²)ₘ₂-.

Embodiment 17. The compound of any one of Embodiments 1-16, wherein X² is CH.

Embodiment 18. The compound of any one of Embodiments 1-17, wherein X¹ is CH.

Embodiment 19. The compound of any one of Embodiments 1-18, wherein X^{a} is N; and X^{b} is CH.

Embodiment 20. The compound of any one of Embodiments 1-18, wherein X^{a} is CH; and X^{b} is CH.

Embodiment 21. The compound of any one of Embodiments 1-20, wherein R⁶ is halo (e.g., —F, —Cl, —Br).

Embodiment 22. The compound of any one of Embodiments 1-21, wherein R⁶ is —F.

Embodiment 23. The compound of any one of Embodiments 1-21, wherein R⁶ is —Cl.

Embodiment 24. The compound of any one of Embodiments 1-23, wherein each R² is H.

Embodiment 25. The compound of any one of Embodiments 1-24, wherein m3 is 1; and X³ is C₁₋₃ alkylene (e.g., methylene, ethylene, or isopropylene).

Embodiment 26. The compound of any one of Embodiments 1-24, wherein m3 is 0.

Embodiment 27. The compound of any one of Embodiments 1-26, wherein R¹ is H.

Embodiment 28. The compound of any one of Embodiments 1-24, wherein m3 is 1; X³ is C₁₋₃ alkylene optionally substituted with 1-3 R^{c}; and R¹ is H.

Embodiment 29. The compound of any one of Embodiments 1-24, wherein m3 is 1; X³ is C₁₋₃ alkylene; and R¹ is H.

Embodiment 30. The compound of any one of Embodiments 1-24, wherein —(X³)ₘ₃—R¹ is methyl, ethyl, or isopropyl (e.g., methyl).

Embodiment 31. The compound of any one of Embodiments 1-12, wherein TBM is

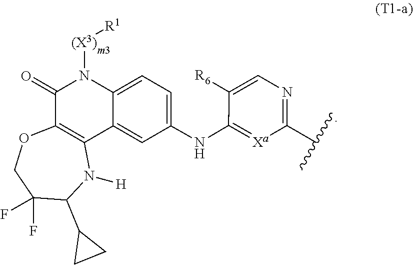

Embodiment 32. The compound of Embodiment 31, wherein m3 is 1; X³ is C₁₋₃ alkylene; and R¹ is H.

Embodiment 33. The compound of Embodiments 31 or 32, wherein —(X³)ₘ₃—R¹ is methyl, ethyl, or isopropyl.

Embodiment 34. The compound of any one of Embodiments 31-33, wherein X^{a} is CH.

Embodiment 35. The compound of any one of Embodiments 31-33, wherein X^{a} is N.

Embodiment 36. The compound of any one of Embodiments 31-35, wherein R⁶ is —F or —Cl.

Embodiment 37. The compound of any one of Embodiments 1-36, wherein L is -(LA)ₙ₁-, wherein LA and n1 are defined according to (AA).

Embodiment 38. The compound of any one of Embodiments 1-37, wherein n1 is an integer from 1 to 5.

Embodiment 39. The compound of any one of Embodiments 1-38, wherein n1 is an integer from 2 to 4 (e.g., 2 or 3).

Embodiment 40. The compound of any one of Embodiments 1-39, wherein L is selected from the group consisting of:
L^{A4}-L^{A1}-L^{A4}-_{bb};
L^{A4}-L^{A4}-_{bb};
L^{A4}-L^{A1}-L^{A1}-L^{A4}-_{bb};
L^{A4}-L^{A3}-L^{A4}-_{bb}; and
L^{A4}-L^{A1}-L^{A4}-L^{A3}-_{bb},
wherein bb represents the point of attachment to Ring C.

Embodiment 41. The compound of Embodiment 40, wherein each L^{A4} is independently C₃₋₁₀ cycloalkylene or 4-12 membered heterocyclylene, each of which is optionally substituted with 1-6 R^{a}.

Embodiment 42. The compound of Embodiments 40 or 41, wherein each L^{A4} is independently 4-12 (e.g., 4-10) membered heterocyclylene optionally substituted with 1-3 R^{a}.

Embodiment 43. The compound of any one of Embodiments 40-42, wherein each L^{A4} is independently monocyclic 4-6 membered nitrogen-containing heterocyclylene optionally substituted with 1-3 R^{a}.

Embodiment 44. The compound of any one of Embodiments 40-42, wherein one L^{A4} is monocyclic 4-6 membered nitrogen-containing heterocyclylene optionally substituted with 1-3 R^{a}; and the other L^{A4} is bicyclic 6-12 (e.g., 6-10) membered nitrogen-containing heterocyclylene optionally substituted with 1-3 R^{a}.

Embodiment 45. The compound of any one of Embodiments 40-42, wherein one L^{A4} is monocyclic 4-6 membered nitrogen-containing heterocyclylene optionally substituted with 1-3 R^{a}; and the other L^{A4} is bicyclic spirocyclic 6-12 (e.g., 6-10) membered nitrogen-containing heterocyclylene optionally substituted with 1-3 R^{a}.

Embodiment 46. The compound of any one of Embodiments 43-45, wherein each L^{A4} contains 1-2 ring nitrogen atoms and no additional ring heteroatoms.

Embodiment 47. The compound of any one of Embodiments 41-46, wherein each R^{a} present on L^{A4} is independently selected from the group consisting of: —F, CN, C₁₋₃ alkoxy, OH, and C₁₋₃ alkyl optionally substituted with 1-3 F.

Embodiment 48. The compound of any one of Embodiments 43-47, wherein L is -L^{A4}-L^{A1}-L^{A4}-_{bb}.

Embodiment 49. The compound of Embodiment 48, wherein L^{A1} is —CH₂—, —CHMe-, or —CMe₂-.

Embodiment 50. The compound of any one of Embodiments 43-47, wherein L is -L^{A4}-L^{A3}-L^{A4}-_{bb}.

Embodiment 51. The compound of Embodiment 50, wherein L^{A3} is —C(═O).

Embodiment 52. The compound of any one of Embodiments 43-47, wherein L is -L^{A4}-L^{A1}-L^{A4}-L^{A3}-_{bb}, and L^{A3} is C(═O).

Embodiment 53. The compound of any one of Embodiments 43-47, wherein L is -L^{A4}-L^{A1}-L^{A1}-L^{A4}-_{bb}, and one or both of L^{A1} is CH₂.

Embodiment 54. The compound of any one of Embodiments 1-39, wherein L is selected from the group consisting of:
L^{A4}-L^{A3}-_{bb};
L^{A4}-L^{A1}-_{bb}; and
L^{A4}-L^{A1}-L^{A3}-_{bb},
wherein bb represents the point of attachment to Ring C.

Embodiment 55. The compound of Embodiment 54, wherein L is -L^{A4}-L^{A3}-_{bb}.

Embodiment 56. The compound of Embodiment 55, wherein L^{A3} is —NH— or —N(C₁₋₃ alkyl)- (e.g., —NH—).

Embodiment 57. The compound of any one of Embodiments 54-56, wherein L^{A4} is 4-12 membered heterocyclylene optionally substituted with 1-6 R^{a}.

Embodiment 58. The compound of any one of Embodiments 54-57, wherein L^{A4} is 4-12 (e.g., 6-10) membered nitrogen-containing heterocyclylene optionally substituted with 1-3 R^{a}.

Embodiment 59. The compound of any one of Embodiments 54-58, wherein L^{A4} is bicyclic spirocyclic 6-12 (e.g., 6-10) membered nitrogen-containing heterocyclylene optionally substituted with 1-3 R^{a}.

Embodiment 60. The compound of any one of Embodiments 57-59, wherein L^{A4} contains 1-2 ring nitrogen atoms and no additional ring heteroatoms.

Embodiment 61. The compound of any one of Embodiments 57-60, wherein each R^{a} present on L^{A4} is independently selected from the group consisting of: —F, CN, C₁₋₃ alkoxy, OH, and C₁₋₃ alkyl optionally substituted with 1-3 F.

Embodiment 62. The compound of any one of Embodiments 1-40, wherein L is selected from the group consisting of the moieties delineated in Table L1-a:

TABLE L1-a

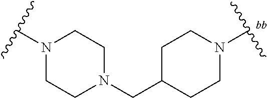

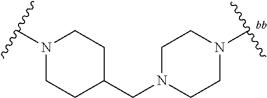

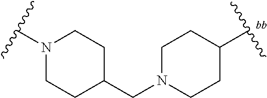

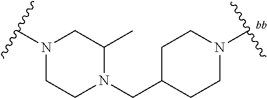

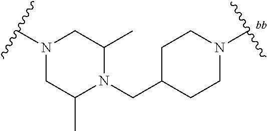

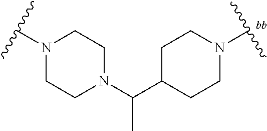

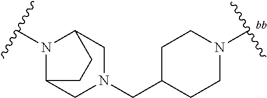

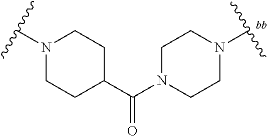

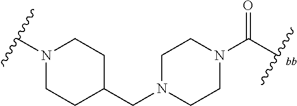

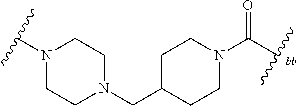

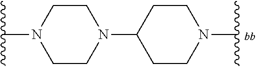

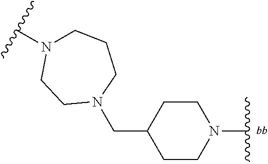

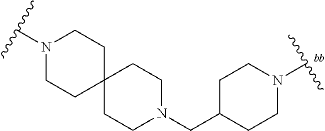

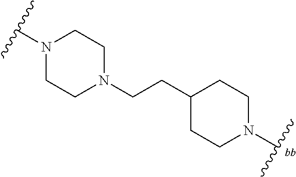

wherein bb represents the point of attachment to Ring C.

Embodiment 63. The compound of any one of Embodiments 1-39 or 54-55, wherein L is selected from the group consisting of the moieties delineated in Table L2-a:

TABLE L2-a

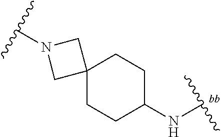

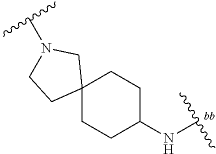

wherein bb represents the point of attachment to Ring C.

Embodiment 64. The compound of Embodiment 1, wherein the compound is a compound of Formula (I-a):

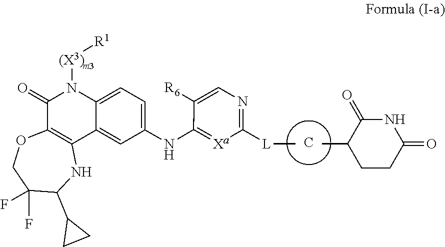

or a pharmaceutically acceptable salt thereof, wherein:
X^{a} is N or CH;
R⁶ is —F or —Cl;
m3 is 1, X³ is C₁₋₃ alkylene, and R¹ is H;

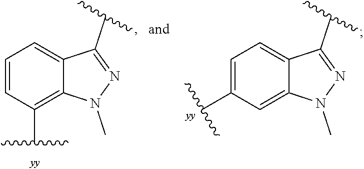

Ring C is selected from the group consisting of: yy and yy;
L is selected from the group consisting of:
   L^{A4}-L^{A1}-L^{A4}-_{bb};
   L^{A4}-L^{A1}-L^{A1}-L^{A4}-_{bb};
   L^{A4}-C(═O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 65. The compound of Embodiment 64, wherein each L^{A4} is independently monocyclic 4-6 membered nitrogen-containing heterocyclylene optionally substituted with 1-3 R^{a},
wherein each L^{A4} contains 1-2 ring nitrogen atoms and no additional ring heteroatoms.

Embodiment 66. The compound of Embodiment 64, wherein one L^{A4} is monocyclic 4-6 membered nitrogen-containing heterocyclylene optionally substituted with 1-3 R^{a}; and the other L^{A4} is bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

Embodiment 67. The compound of any one of Embodiments 64-66, wherein L is -L^{A4}-L^{A1}-L^{A4}-_{bb}; and L^{A1} is CH₂ or CHMe.

Embodiment 68. The compound of Embodiment 64, wherein L is selected from the group consisting of the moieties delineated in Table L1-a:

TABLE L1-a

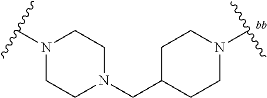

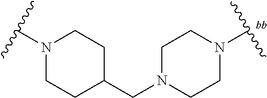

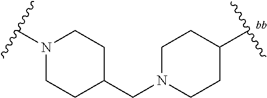

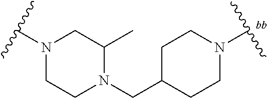

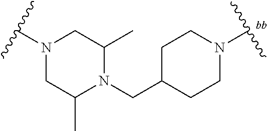

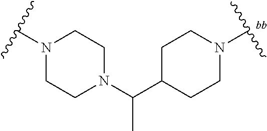

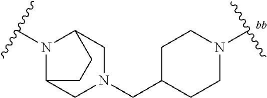

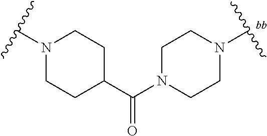

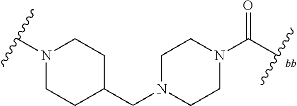

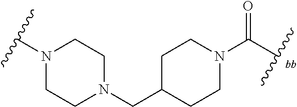

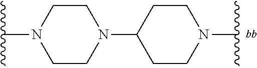

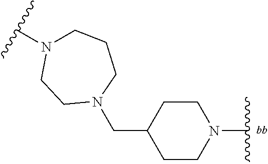

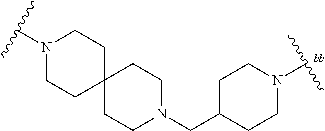

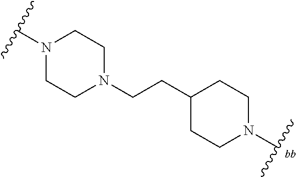

wherein bb represents the point of attachment to Ring C.

Embodiment 69. The compound of Embodiment 1, wherein the compound is a compound of Formula (I-b):

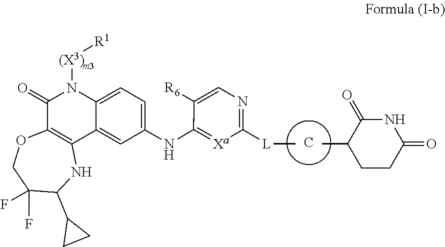

or a pharmaceutically acceptable salt thereof, wherein:
X^{a} is N or CH;
R⁶ is —F or —Cl;
m3 is 1, X³ is C₁₋₃ alkylene, and R¹ is H;

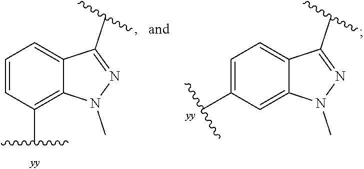

Ring C is selected from the group consisting of: yy and 1′;
L is -L^{A4}-L^{A3}-_{bb} or -L^{A4}-L^{A1}-L^{A3}-_{bb}, wherein bb represents the point of attachment to Ring C; and
L^{A4} is 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 70. The compound of Embodiment 69, wherein L is -L^{A4}-L^{A3}-_{bb}, and L^{A3} is —NH—.

Embodiment 71. The compound of Embodiments 69 or 70, wherein L^{A4} is monocyclic 4-6 membered nitrogen-containing heterocyclylene optionally substituted with 1-3 R^{a}, wherein L^{A4} contains 1-2 ring nitrogen atoms and no additional ring heteroatoms.

Embodiment 72. The compound of any one of Embodiments 69-71, wherein L^{A4} is bicyclic spirocyclic 6-12 (e.g., 6-10) membered nitrogen-containing heterocyclylene optionally substituted with 1-3 R^{a},
wherein L^{A4} contains 1-2 ring nitrogen atoms and no additional ring heteroatoms.

Embodiment 73. The compound of any one of Embodiments 64-72, wherein X^{a} is N.

Embodiment 74. The compound of any one of Embodiments 64-72, wherein X^{a} is CH.

Embodiment 75. The compound of Embodiment 1, wherein the compound is selected from the group consisting of Compound No. 101, 101a, 102, 102a, 130, 130a, 132, 132a, 214, 214a, 214b, 374, 374a, 379, 379a, 379b, 380, 380a, 380b, 381, 381a, 381b, 382, 382a, 383, 383a, 384, 384a, 385, 385a, 386, 386a, 387, 387a, 388, 388a, 389, 389a, 390, 390a, 391, 391a, 392, 392a, 393, 393a, 394, 394a, 395, 395a, 396, 396a, 397, 397a, 398, 398a, 399, 399a, 400, 400a, 401, 401a, 402, 402a, 403, 403a, 404, 404a, 405, 405a, 406, 406a, 407, 407a, 408, 408a, 409, 409a, 410, 410a, 411, 411a, 412, 412a, 413, 413a, 414, 414a, 415, 415a, 416, and 416a as depicted in Table C1, or a pharmaceutically acceptable salt thereof.

Embodiment 76. A pharmaceutical composition comprising a compound of any one of Embodiments 1-75, or a pharmaceutically acceptable salt thereof, and a pharmaceutically acceptable excipient.

Embodiment 77. A BCL6 protein non-covalently bound with a compound of any one of Embodiments 1-75, or a pharmaceutically acceptable salt thereof.

Embodiment 78. A ternary complex comprising a BCL6 protein, a compound of any one of Embodiments 1-75, or a pharmaceutically acceptable salt thereof, and a CRBN protein.

Embodiment 79. A method for treating a cancer in a subject in need thereof, the method comprising administering to the subject a therapeutically effective amount of a compound of any one of Embodiments 1-75, or a pharmaceutically acceptable salt thereof, or a pharmaceutical composition according to Embodiment 76.

Embodiment 80. The method of Embodiment 79, wherein the cancer is a hematological cancer, breast cancer, gastrointestinal cancer, brain cancer, lung cancer, or a combination thereof.

Embodiment 81. The method of Embodiment 80, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), chronic myeloid leukemia (CML), or a combination thereof.

Embodiment 82. The method of Embodiment 81, wherein the hematological cancer is DLBCL.

Embodiment 83. The method of any one of Embodiments 79-82, further comprising administering an additional therapy or therapeutic agent to the subject.

Embodiment 84. The method of Embodiment 83, wherein the additional therapy or therapeutic agent is a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, or a combination thereof.

Embodiment 85. A method for treating an autoimmune condition in a subject in need thereof, the method comprising administering to the subject a therapeutically effective amount of a compound of any one of Embodiments 1-75, or a pharmaceutically acceptable salt thereof, or a pharmaceutical composition according to Embodiment 76.

Embodiment 86. A method for treating a lymphoproliferative disorder in a subject in need thereof, the method comprising administering to the subject a therapeutically effective amount of a compound of any one of Embodiments 1-75, or a pharmaceutically acceptable salt thereof, or a pharmaceutical composition according to Embodiment 76.

Embodiment 87. A method for inducing degradation of a BCL6 protein in a mammalian cell, the method comprising contacting the mammalian cell with an effective amount of a compound of any one of Embodiments 1-75, or a pharmaceutically acceptable salt thereof.

Embodiment 88. The method of Embodiment 87, wherein the contacting occurs in vivo.

Embodiment 89. The method of Embodiment 87, wherein the contacting occurs in vitro.

Embodiment 90. The method of any one of Embodiments 87-89, wherein the mammalian cell is a mammalian cancer cell.

### P06 Embodiments

Embodiment 1. A compound of Formula (I):

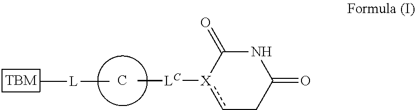

or a pharmaceutically acceptable salt thereof, wherein:
TBM is (T1):

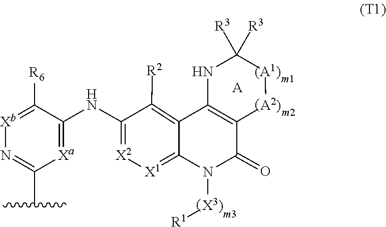

X¹ is selected from the group consisting of N and CR²;
X² is CH;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
m3 is 0 or 1;
X³ is C₁₋₃ alkylene optionally substituted with 1-3 R^{e};
R¹ is selected from the group consisting of: H and C₃₋₆ cycloalkyl;
m1 is 2; each A¹ is independently CH₂, CHR⁴, or CR⁴R⁴;
m2 is 1; A² is —O—;
one R³ is selected from the group consisting of:
(i) C₃₋₆ cycloalkyl optionally substituted with 1-3 R⁹, and
(ii) C₁₋₃ alkyl optionally substituted with 1-3 —F; and
the other R³ is H;
each R⁴ is independently selected from the group consisting of: H, R^{a}, and R^{b};
X^{a} is selected from the group consisting of: N, CH, and CF;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1} L^{A3}, and L^{A4} provided that 1-3 occurrences of L^{A} is L^{A4};

### (BB)

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any 0-0, N-0, N—N, N—S(O)o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{e};
Ring C is selected from the group consisting of:

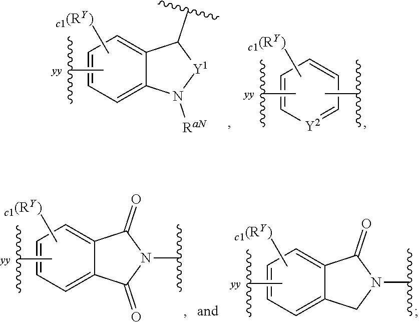

c1 is 0, 1, 2, or 3;
each R^{Y} is independently selected from the group consisting of: R^{a} and R^{b};
R^{a}N is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than 0; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{e};
(f) —NR^{d}R^{e};
(g) C(═O)C₁₋₆ alkyl;
(h) C(═O)C₁₋₆ haloalkyl;
(i) C(═O)OH;
(j) C(═O)OC₁₋₆ alkyl;
(k) C(═O)OC₁₋₆ haloalkyl;
(l) C(═O)N(R^{f})₂;
(m) S(O)₀₋₂(C₁₋₆ alkyl);
(n) S(O)₀₋₂(C₁₋₆ haloalkyl);
(o) S(O)₁₋₂N(R)₂; and
(p) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h}.
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀-3 alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

Embodiment 2. The compound of Embodiment 1, wherein Ring C is

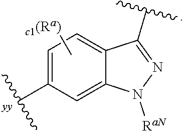

Embodiment 3. The compound of Embodiment 1, wherein Ring C is

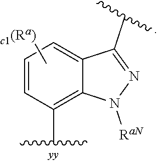

Embodiment 4. The compound of Embodiment 1, wherein Ring C is

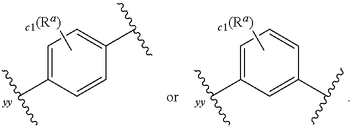

Embodiment 5. The compound of Embodiment 1, wherein Ring C is

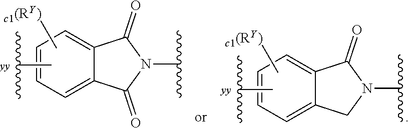

Embodiment 6. The compound of any one of Embodiments 1-5, wherein c1 is 0.

Embodiment 7. The compound of any one of Embodiments 1-5, wherein c1 is 1; and R^{Y} is halo (e.g., —F).

Embodiment 8. The compound of any one of Embodiments 1-3, wherein R^{aN} is C₁₋₃ alkyl (e.g., methyl).

Embodiment 9. The compound of any one of Embodiments 1-8, wherein X is CH.

Embodiment 10. The compound of any one of Embodiments 1-9, wherein L^{C} is a bond.

Embodiment 11. The compound of Embodiment 1, wherein the

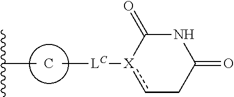

moiety is selected from the group consisting of the moieties delineated in Table CM-1b:

TABLE CM-1b

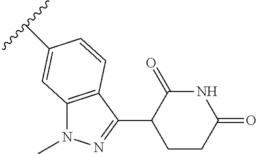 [SMILES: *c1ccc2c(C3CCC(=O)NC3=O)nn(C)c2c1]

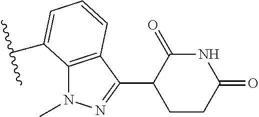 [SMILES: *c1cccc2c(C3CCC(=O)NC3=O)nn(C)c12]

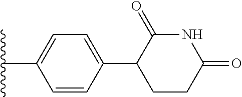 [SMILES: *c1ccc(C2CCC(=O)NC2=O)cc1]

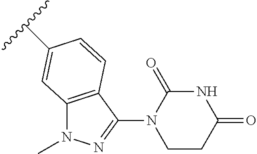 [SMILES: *c1ccc2c(N3CCC(=O)NC3=O)nn(C)c2c1]

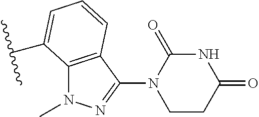 [SMILES: *c1cccc2c(N3CCC(=O)NC3=O)nn(C)c12]

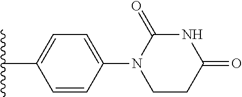 [SMILES: *c1ccc(N2CCC(=O)NC2=O)cc1]

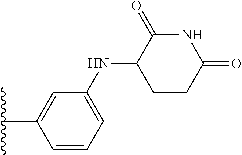 [SMILES: *c1cccc(NC2CCC(=O)NC2=O)c1]

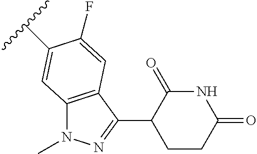 [SMILES: *c1cc2c(cc1F)c(C1CCC(=O)NC1=O)nn2C]

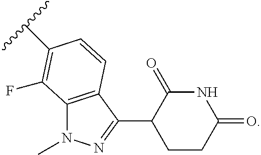 [SMILES: *c1ccc2c(C3CCC(=O)NC3=O)nn(C)c2c1F]

Embodiment 12. The compound of Embodiment 11, wherein

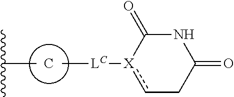

moeity is

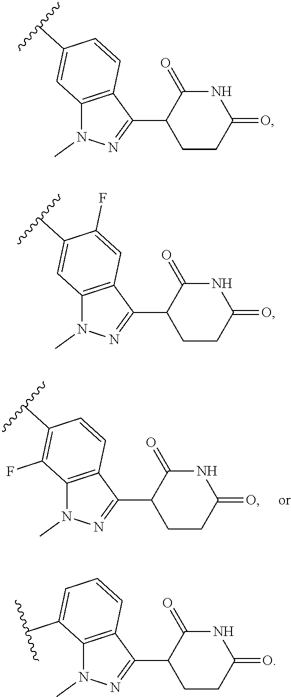 [SMILES: *c1ccc2c(C3CCC(=O)NC3=O)nn(C)c2c1 ; *c1cc2c(cc1F)c(C1CCC(=O)NC1=O)nn2C ; *c1ccc2c(C3CCC(=O)NC3=O)nn(C)c2c1F ; *c1cccc2c(C3CCC(=O)NC3=O)nn(C)c12]

Embodiment 13. The compound of any one of Embodiments 1-12, wherein -(A¹)ₘ₁- is —C(R⁴R⁴)—CH₂—* (e.g., —CF₂—CH₂—*), wherein * represents the point of attachment to -(A²)ₘ₂-.

Embodiment 14. The compound of any one of Embodiments 1-13, wherein one R³ is C₃₋₆ cycloalkyl; and the other R³ is H.

Embodiment 15. The compound of any one of Embodiments 1-14, wherein one R³ is cyclopropyl; and the other R³ is H.

Embodiment 16. The compound of any one of Embodiments 1-15, wherein one R³ is cyclopropyl; the other R³ is H; and -(A¹)ₘ₁- is —CF₂—CH₂—*, wherein * represents the point of attachment to -(A²)ₘ₂-.

Embodiment 17. The compound of any one of Embodiments 1-16, wherein X² is CH.

Embodiment 18. The compound of any one of Embodiments 1-17, wherein X¹ is CH.

Embodiment 19. The compound of any one of Embodiments 1-18, wherein X^{a} is N; and X^{b} is CH.

Embodiment 20. The compound of any one of Embodiments 1-18, wherein X^{a} is CH; and X^{b} is CH.

Embodiment 21. The compound of any one of Embodiments 1-20, wherein R⁶ is halo (e.g., —F, —Cl, —Br) or CN.

Embodiment 22. The compound of any one of Embodiments 1-21, wherein R⁶ is —F.

Embodiment 23. The compound of any one of Embodiments 1-21, wherein R⁶ is —Cl.

Embodiment 24. The compound of any one of Embodiments 1-23, wherein each R² is H.

Embodiment 25. The compound of any one of Embodiments 1-24, wherein m3 is 1; and X³ is C₁₋₃ alkylene (e.g., methylene, ethylene, or isopropylene).

Embodiment 26. The compound of any one of Embodiments 1-24, wherein m3 is 0.

Embodiment 27. The compound of any one of Embodiments 1-26, wherein R¹ is H.

Embodiment 28. The compound of any one of Embodiments 1-24, wherein m3 is 1; X³ is C₁₋₃ alkylene optionally substituted with 1-3 R^{c}; and R¹ is H.

Embodiment 29. The compound of any one of Embodiments 1-24, wherein m3 is 1; X³ is C₁₋₃ alkylene; and R¹ is H.

Embodiment 30. The compound of any one of Embodiments 1-24, wherein —(X³)ₘ₃—R¹ is methyl, ethyl, or isopropyl (e.g., methyl).

Embodiment 31. The compound of any one of Embodiments 1-12, wherein TBM is

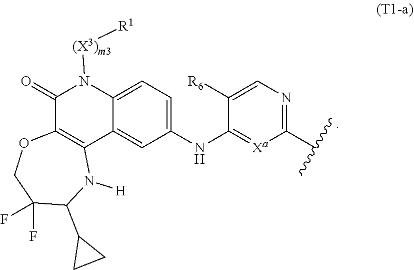

Embodiment 32. The compound of Embodiment 31, wherein m3 is 1; X³ is C₁₋₃ alkylene; and R¹ is H.

Embodiment 33. The compound of Embodiments 31 or 32, wherein —(X³)ₘ₃—R¹ is methyl, ethyl, or isopropyl.

Embodiment 34. The compound of any one of Embodiments 31-33, wherein X^{a} is CH.

Embodiment 35. The compound of any one of Embodiments 31-33, wherein X^{a} is N.

Embodiment 36. The compound of any one of Embodiments 31-35, wherein R⁶ is —F or —Cl.

Embodiment 37. The compound of any one of Embodiments 1-36, wherein L is -(L^{A})ₙ₁-, wherein L^{A} and n1 are defined according to (AA).

Embodiment 38. The compound of any one of Embodiments 1-37, wherein n1 is an integer from 1 to 5.

Embodiment 39. The compound of any one of Embodiments 1-38, wherein n1 is an integer from 2 to 4 (e.g., 2 or 3).

Embodiment 40. The compound of any one of Embodiments 1-39, wherein L is selected from the group consisting of:
L^{A4}-L^{A1}-L^{A4}-_{bb};
L^{A4}-L^{A4}-_{bb};
L^{A4}-L^{A1}-L^{A1}-L^{A4}-_{bb};
L^{A4}-L^{A3}-L^{A4}-_{bb}; and
L^{A4}-L^{A1}-L^{A4}-L^{A3}-_{bb},
wherein bb represents the point of attachment to Ring C.

Embodiment 41. The compound of Embodiment 40, wherein each L^{A4} is independently C₃₋₁₀ cycloalkylene or 4-12 membered heterocyclylene, each of which is optionally substituted with 1-6 R^{a}.

Embodiment 42. The compound of Embodiments 40 or 41, wherein each L^{A4} is independently 4-12 (e.g., 4-10) membered heterocyclylene optionally substituted with 1-3 R^{a}.

Embodiment 43. The compound of any one of Embodiments 40-42, wherein each L^{A4} is independently monocyclic 4-6 membered nitrogen-containing heterocyclylene optionally substituted with 1-3 R^{a}.

Embodiment 44. The compound of any one of Embodiments 40-42, wherein one L^{A4} is monocyclic 4-6 membered nitrogen-containing heterocyclylene optionally substituted with 1-3 R^{a}; and the other L^{A4} is bicyclic 6-12 (e.g., 6-10) membered nitrogen-containing heterocyclylene optionally substituted with 1-3 R^{a}.

Embodiment 45. The compound of any one of Embodiments 40-42, wherein one L^{A4} is monocyclic 4-6 membered nitrogen-containing heterocyclylene optionally substituted with 1-3 R^{a}; and the other L^{A4} is bicyclic spirocyclic 6-12 (e.g., 6-10) membered nitrogen-containing heterocyclylene optionally substituted with 1-3 R^{a}.

Embodiment 46. The compound of any one of Embodiments 43-45, wherein each L^{A4} contains 1-2 ring nitrogen atoms and no additional ring heteroatoms.

Embodiment 47. The compound of any one of Embodiments 41-46, wherein each R^{a} present on L^{A4} is independently selected from the group consisting of: —F, CN, C₁₋₃ alkoxy, OH, and C₁₋₃ alkyl optionally substituted with 1-3 F.

Embodiment 48. The compound of any one of Embodiments 43-47, wherein L is -L^{A4}-L^{A1}-L^{A4}-_{bb}.

Embodiment 49. The compound of Embodiment 48, wherein L^{A1} is —CH₂—, —CHMe-, or —CMe₂-.

Embodiment 50. The compound of any one of Embodiments 43-47, wherein L is -L^{A4}-L^{A3}-L^{A4}-_{bb}.

Embodiment 51. The compound of Embodiment 50, wherein L^{A3} is —C(═O).

Embodiment 52. The compound of any one of Embodiments 43-47, wherein L is -L^{A4}-L^{A1}-L^{A4}-L^{A3}-_{bb}, and L^{A3} is C(═O).

Embodiment 53. The compound of any one of Embodiments 43-47, wherein L is -L^{A4}-L^{A1}-L^{A1}-L^{A4}-_{bb}, and one or both of L^{A1} is CH₂.

Embodiment 54. The compound of any one of Embodiments 1-39, wherein L is selected from the group consisting of:
L^{A4}-L^{A3}-_{bb};
L^{A4}-L^{A1}-_{bb}; and
L^{A4}-L^{A1}-L^{A3}-_{bb},
wherein bb represents the point of attachment to Ring C.

Embodiment 55. The compound of Embodiment 54, wherein L is -L^{A4}-L^{A3}-_{bb}.

Embodiment 56. The compound of Embodiment 55, wherein L^{A3} is —NH— or —N(C₁₋₃ alkyl)- (e.g., —NH—).

Embodiment 57. The compound of any one of Embodiments 54-56, wherein L^{A4} is 4-12 membered heterocyclylene optionally substituted with 1-6 R^{a}.

Embodiment 58. The compound of any one of Embodiments 54-57, wherein L^{A4} is 4-12 (e.g., 6-10) membered nitrogen-containing heterocyclylene optionally substituted with 1-3 R^{a}.

Embodiment 59. The compound of any one of Embodiments 54-58, wherein L^{A4} is bicyclic spirocyclic 6-12 (e.g., 6-10) membered nitrogen-containing heterocyclylene optionally substituted with 1-3 R^{a}.

Embodiment 60. The compound of any one of Embodiments 57-59, wherein L^{A4} contains 1-2 ring nitrogen atoms and no additional ring heteroatoms.

Embodiment 61. The compound of any one of Embodiments 57-60, wherein each R^{a} present on L^{A4} is independently selected from the group consisting of: —F, CN, C₁₋₃ alkoxy, OH, and C₁₋₃ alkyl optionally substituted with 1-3 F.

Embodiment 62. The compound of any one of Embodiments 1-40, wherein L is selected from the group consisting of the moieties delineated in Table L1-a:

TABLE L1-a

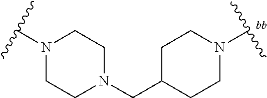

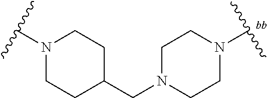

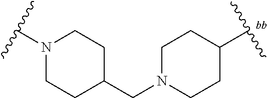

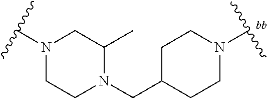

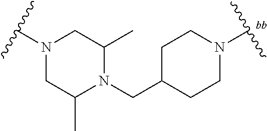

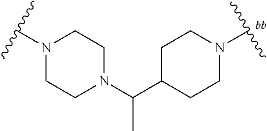

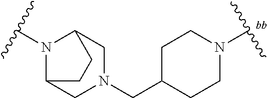

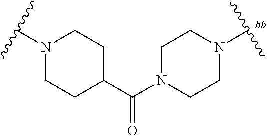

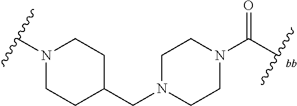

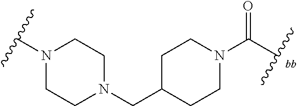

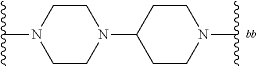

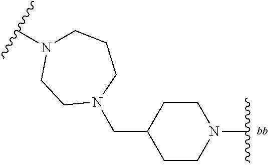

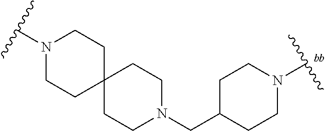

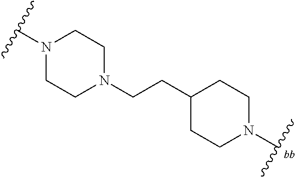

wherein bb represents the point of attachment to Ring C.

Embodiment 63. The compound of any one of Embodiments 1-39 or 54-55, wherein L is selected from the group consisting of the moieties delineated in Table L2-a:

TABLE L2-a

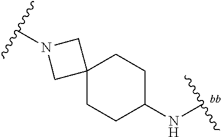

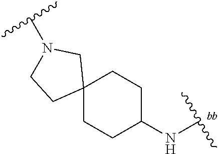

wherein bb represents the point of attachment to Ring C.

Embodiment 64. The compound of Embodiment 1, wherein the compound is a compound of Formula (I-a):

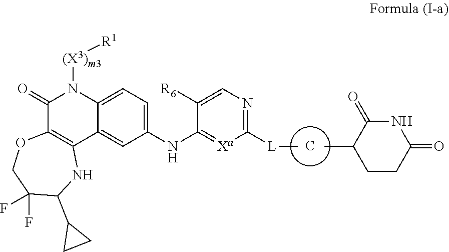

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

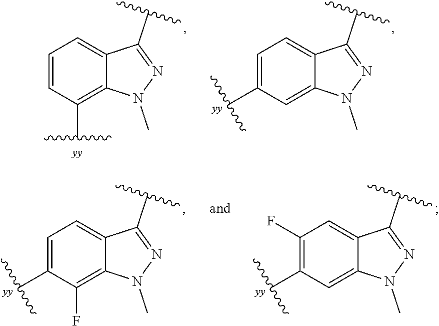 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L is selected from the group consisting of:
   L^{A4}-L^{A1}-L^{A4}-_{bb};
   L^{A4}-L^{A1}-L^{A1}-L^{A4}-_{bb};
   L^{A4}-L^{A4}-_{bb};
   L^{A4}-C(O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 65. The compound of Embodiment 64, wherein each L^{A4} is independently monocyclic 4-6 membered nitrogen-containing heterocyclylene optionally substituted with 1-3 R^{a},
wherein each L^{A4} contains 1-2 ring nitrogen atoms and no additional ring heteroatoms.

Embodiment 66. The compound of Embodiment 64, wherein one L^{A4} is monocyclic 4-6 membered nitrogen-containing heterocyclylene optionally substituted with 1-3 R^{a}; and the other L^{A4} is bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

Embodiment 67. The compound of any one of Embodiments 64-66, wherein L is -L^{A4}-L^{A1}-L^{A4}-_{bb}; and L^{A1} is CH₂ or CHMe.

Embodiment 68. The compound of Embodiment 64, wherein L is selected from the group consisting of the moieties delineated in Table L1-a.

Embodiment 69. The compound of Embodiment 1, wherein the compound is a compound of Formula (I-b):

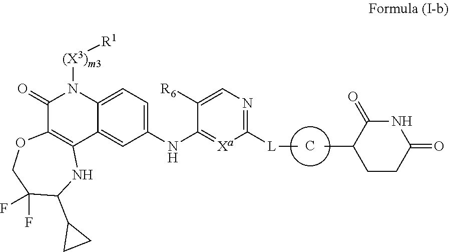

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

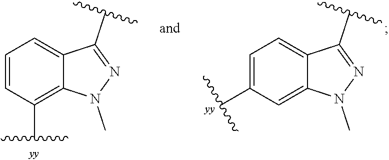 [SMILES: *c1nn(C)c2c(*)cccc12 ; *c1ccc2c(*)nn(C)c2c1]

L is -L^{A4}-L^{A3}-_{bb} or -L^{A4}-L^{A1}-L^{A3}-_{bb}, wherein bb represents the point of attachment to Ring C; and
L^{A4} is 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 70. The compound of Embodiment 69, wherein L is -L^{A4}-L^{A3}-_{bb}, and L^{A3} is —NH—.

Embodiment 71. The compound of Embodiments 69 or 70, wherein L^{A4} is monocyclic 4-6 membered nitrogen-containing heterocyclylene optionally substituted with 1-3 R^{a},
wherein L^{A4} contains 1-2 ring nitrogen atoms and no additional ring heteroatoms.

Embodiment 72. The compound of any one of Embodiments 69-71, wherein L^{A4} is bicyclic spirocyclic 6-12 (e.g., 6-10) membered nitrogen-containing heterocyclylene optionally substituted with 1-3 R^{a},
wherein L^{A4} contains 1-2 ring nitrogen atoms and no additional ring heteroatoms.

Embodiment 73. The compound of any one of Embodiments 64-72, wherein X^{a} is N.

Embodiment 74. The compound of any one of Embodiments 64-72, wherein X^{a} is CH.

Embodiment 75. The compound of Embodiment 1, wherein the compound is selected from the group consisting of Compound No. 101, 101a, 102, 102a, 130, 130a, 132, 132a, 133, 133a, 170,170a, 186,186a, 194,194a, 214, 214a, 214b, 374, 374a, 375, 375a, 379, 379a, 379b, 380, 380a, 380b, 381, 381a, 381b, 382, 382a, 383, 383a, 384, 384a, 385, 385a, 386, 386a, 387, 387a, 388, 388a, 389, 389a, 390, 390a, 391, 391a, 392, 392a, 393, 393a, 394, 394a, 395, 395a, 396, 396a, 397, 397a, 398, 398a, 399, 399a, 400, 400a, 401, 401a, 402, 402a, 403, 403a, 404, 404a, 405, 405a, 406, 406a, 407, 407a, 408, 408a, 409, 409a, 410, 410a, 411, 411a, 412, 412a, 413, 413a, 414, 414a, 415, 415a, 416, 416a, 417, 417a, 418, 418a, 419, 419a, 420, 420a, 421, 421a, 422, 422a, 423, 423a, 424, 424a, 425, 425a, 426, 426a, 426b, 427, 427a, 428, 428a, 429, 429a, 430, 430a, 431, 431a, 432, 432a, 433, 433a, 434, 434a, 435, 435a, 436, 436a, 437, 437a, 438, 438a, 439, 439a, 440, 440a, 441, 441a, 442, 442a, 442b, 443, 443a, 443b, 444, 444a, 445, 445a, 446, 446a, 447, 447a, 448, 448a, 449, 449a, 450, 450a, 451, 451a, 451b, 452, 452a, 453, 453a, 454, 454a, 455, 455a, 456, 456a, 457, 457a, 458, 458a, 459, 459a, 459b, 460, 460a, 460b, 461, 461a, 462, 462a, 463, 463a, 464, 464a, 464b, 464c, 464d, 465, 465a, 465b, 465c, 465d, 466, 466a, 466b, 467, 467a, 468, 468a, 469, 469a, 470, 470a, 471, 471a, 472, 472a, 473, 473a, 474, 474a, 475, 475a, 475b, 476, 476a, 476b, 477, 477a, 478, 478a, 479, 479a, 480, 480a, 481, 481a, 481b, 482, 482a, 482b, 483, 483a, 484, 484a, 485, 485a, 486, 486a, 486b, 486c, 486d, 487, 487a, 487b, 487c, 487d, 488, 488a, 489, 489a, 490, 490a, 491, 491a, 491b, 492, 492a, 492b, 492c, 492d, 493, 493a, 493b, 494, 494a, 495, 495a, 496, 496a, 496b, 497, 497a, 498, 498a, 499, 499a, 500, 500a, 500b, 501, 501a, 502, 502a, 503, 503a, 504, 504a, 505, 505a, 506, 506a, 506b, 507, 507a, 508, 508a, 509, 509a, 510, 510a, 511, 511a, 512, 512a, 512b, 513, 513a, 514, 514a, 516, 516a, 518, 518a, 519, 519a, 520, 520a, 521, 521a, 522, 522a, 522b, 523, 523a, 523b, 524, 524a, 525, 525a, 525b, 525c, 525d, 526, 526a, 527, 527a, 527b, 528, 528a, 529, 529a, 529b, 529c, 530, 530a, 531, 531a, 532, 532a, 533, 533a, 534, 534a, 534b, 534c, 534d, 535, 535a, 536, 536a, 537, 537a, 538, 538a, 538b, 538c, 539, 539a, 540, 540a, 541, 541a, 542, 542a, 543, 543a, 544, 544a, 545, 545a, 546, 546a, 547, 547a, 548, 548a, 548b, 549, 549a, 550, 550a, 551, 551a, 552, 552a, 553, 553a, 554, 554a, 555, 555a, 556, 556a, 557, 557a, 558, 558a, 559, 559a, 559b, 560, 560a, 560b, 561, 561a, 562, 562a, 563, 563a, 563b, 564, 564a, 565, 565a, 566, 566a, 568, 568a, 568b, 569, 569a, 570, 570a, 572, 572a, 573, 573a, 574, 574a, 575, 575a, 575b, 576, 576a, 577, 577a, 578, 578a, 578b, 579, 579a, 579b, 581, 581a, 582, 582a, and 582b as depicted in Table C1, or a pharmaceutically acceptable salt thereof.

Embodiment 76. A pharmaceutical composition comprising a compound of any one of Embodiments 1-75, or a pharmaceutically acceptable salt thereof, and a pharmaceutically acceptable excipient.

Embodiment 77. A BCL6 protein non-covalently bound with a compound of any one of Embodiments 1-75, or a pharmaceutically acceptable salt thereof.

Embodiment 78. A ternary complex comprising a BCL6 protein, a compound of any one of Embodiments 1-75, or a pharmaceutically acceptable salt thereof, and a CRBN protein.

Embodiment 79. A method for treating a cancer in a subject in need thereof, the method comprising administering to the subject a therapeutically effective amount of a compound of any one of Embodiments 1-75, or a pharmaceutically acceptable salt thereof, or a pharmaceutical composition according to Embodiment 76.

Embodiment 80. The method of Embodiment 79, wherein the cancer is a hematological cancer, breast cancer, gastrointestinal cancer, brain cancer, lung cancer, or a combination thereof.

Embodiment 81. The method of Embodiment 80, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML).

Embodiment 82. The method of Embodiment 81, wherein the hematological cancer is selected from the group consisting of DLBCL, FL, MCL, BL, PTCL, and ALL (e.g., B-ALL).

Embodiment 83. The method of Embodiment 81, wherein the hematological cancer is FL or DLBCL.

Embodiment 84. The method of Embodiment 81, wherein the hematological cancer is DLBCL.

Embodiment 85. The method of Embodiment 81, wherein the hematological cancer is FL.

Embodiment 86. The method of Embodiment 81, wherein the hematological cancer is BL.

Embodiment 87. The method of Embodiment 81, wherein the hematological cancer is a PTCL.

Embodiment 88. The method of Embodiment 81, wherein, the hematological cancer is ALL (e.g., B-ALL).

Embodiment 89. The method of any one of Embodiments 79-88, wherein the therapeutically effective amount of a compound of any one of Embodiments 1-75, or a pharmaceutically acceptable salt thereof, or a pharmaceutical composition according to

Embodiment 76, is administered to the subject as a monotherapy.

Embodiment 90. The method of any one of Embodiments 79-88, comprising administering an additional therapy or therapeutic agent to the subject.

Embodiment 91. The method of Embodiment 90, wherein the additional therapy or therapeutic agent is a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, or a combination thereof.

Embodiment 92. The method of Embodiment 91, wherein the additional therapy or therapeutic agent is an Abl inhibitor.

Embodiment 93. The method of Embodiment 92, wherein the cancer is a B-ALL (e.g., a Philadelphia chromosome positive B-ALL or Philadelphia chromosome-like B-ALL).

Embodiment 94. The method of Embodiment 91, wherein the additional therapy or therapeutic agent is a BTK inhibitor.

Embodiment 95. The method of Embodiment 94, wherein the cancer is a B-ALL (e.g., a pre-BCR+B-ALL or a B-ALL dependent on R^{a5} signaling).

Embodiment 96. The method of Embodiment 91, wherein the additional therapy or therapeutic agent is a JAK inhibitor.

Embodiment 97. The method of Embodiment 96, wherein the cancer is a B-ALL (e.g., a JAK2 (e.g., JAK2^{R683G} or JAK2^{I682}F) mutant B-ALL, with or without high CRLF2 expression).

Embodiment 98. The method of Embodiment 91, wherein the additional therapy or therapeutic agent is a BCL-2 inhibitor.

Embodiment 99. The method of Embodiment 98, wherein the cancer is selected from the group consisting of DLBCL, FL, MCL, and ALL (e.g., B-ALL).

Embodiment 100. The method of Embodiment 98, wherein the cancer is a DLBCL.

Embodiment 101. The method of Embodiment 98, wherein the cancer is a FL.

Embodiment 102. The method of Embodiment 98, wherein the cancer is an MCL.

Embodiment 103. The method of Embodiment 98, wherein the cancer is a B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, or a BCL2 amplified B-ALL).

Embodiment 104. The method of Embodiment 91, wherein the additional therapy or therapeutic agent is an inhibitor of the PRC2.

Embodiment 105. The method of Embodiment 104, wherein the cancer is selected from the group consisting of DLBCL, FL, MCL, and ALL (e.g., B-ALL).

Embodiment 106. The method of Embodiment 104, wherein the cancer is a DLBCL.

Embodiment 107. The method of Embodiment 104, wherein the cancer is a FL.

Embodiment 108. The method of Embodiment 104, wherein the cancer is an MCL.

Embodiment 109. The method of Embodiment 104, wherein the cancer is a B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, or a BCL2 amplified B-ALL).

Embodiment 110. The method of Embodiment 91, wherein the additional therapy or therapeutic agent is anti-CD20 therapy.

Embodiment 111. The method of Embodiment 110, wherein the cancer is a DLBCL or a FL.

Embodiment 112. The method of Embodiment 110, wherein the cancer is a DLBCL.

Embodiment 113. The method of Embodiment 110, wherein the cancer is a FL.

Embodiment 114. The method of Embodiment 91, wherein the additional therapy or therapeutic agent is chemotherapy.

Embodiment 115. The method of Embodiment 114, wherein the cancer is B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, a Philadelphia chromosome positive B-ALL, a Philadelphia chromosome-like B-ALL, a pre-BCR+B-ALL, a B-ALL dependent on R^{a5} signaling).

Embodiment 116. A method for treating an autoimmune condition in a subject in need thereof, the method comprising administering to the subject a therapeutically effective amount of a compound of any one of Embodiments 1-75, or a pharmaceutically acceptable salt thereof, or a pharmaceutical composition according to Embodiment 76.

Embodiment 117. A method for treating a lymphoproliferative disorder in a subject in need thereof, the method comprising administering to the subject a therapeutically effective amount of a compound of any one of Embodiments 1-75, or a pharmaceutically acceptable salt thereof, or a pharmaceutical composition according to Embodiment 76.

Embodiment 118. A method for inducing degradation of a BCL6 protein in a mammalian cell, the method comprising contacting the mammalian cell with an effective amount of a compound of any one of Embodiments 1-75, or a pharmaceutically acceptable salt thereof.

Embodiment 119. The method of Embodiment 118, wherein the contacting occurs in vivo.

Embodiment 120. The method of Embodiment 118, wherein the contacting occurs in vitro.

Embodiment 121. The method of any one of Embodiments 118-120, wherein the mammalian cell is a mammalian cancer cell.

### Exemplary Formula (I-aa) and (I-a) Embodiments

Embodiment 1. A compound of Formula (I-aa):

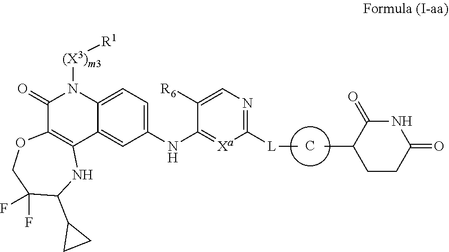

or a pharmaceutically acceptable salt thereof, wherein:
m3 is 0 or 1;
X³ is C₁₋₃ alkylene optionally substituted with 1-3 R^{e};
R¹ is selected from the group consisting of: H and C₃₋₆ cycloalkyl;
X^{a} is N or CH;
R⁶ is —F or —Cl;
m3 is 1, X³ is C₁₋₃ alkylene, and R¹ is H;
Ring C is selected from the group consisting of:

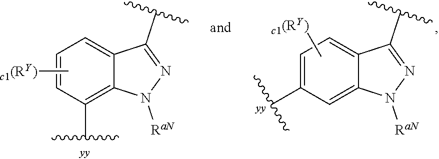

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:
   L^{A4}-L^{A1}-L^{A4}-_{bb};
   L^{A4}-L^{A1}-L^{A1}-L^{A4}-_{bb};
   L^{A4}-L^{A4}-_{bb};
   L^{A4}-C(═O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 2. The compound of Embodiment 1, wherein the compound is a compound of Formula (I-a):

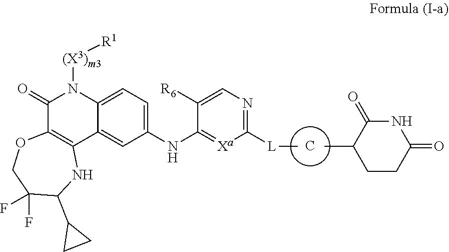

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

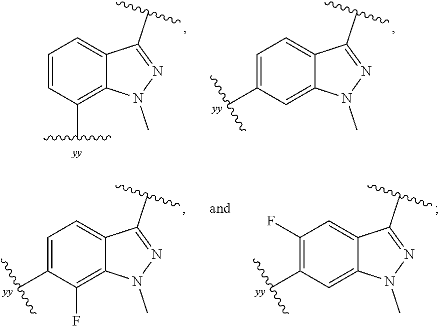 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L is selected from the group consisting of:
   L^{A4}-L^{A1}-L^{A4}-_{bb};
   L^{A4}-L^{A1}-L^{A1}-L^{A4}-_{bb};
   L^{A4}-L^{A4}-_{bb};
   L^{A4}-C(═O)-L^{A4}-_{bb}; and
   L^{A4}-L^{A1}-L^{A4}-C(═O)-_{bb},
wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 3. The compound of Embodiments 1 or 2, wherein Ring C is selected from the group consisting of:

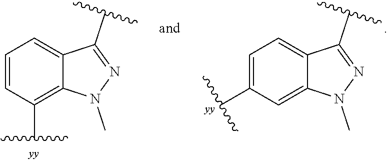 [SMILES: *c1nn(C)c2c(*)cccc12 ; *c1ccc2c(*)nn(C)c2c1]

Embodiment 4. The compound of any one of Embodiments 1-3, wherein L is selected from the group consisting of:
L^{A4}-L^{A1}-L^{A4}-_{bb};
L^{A4}-L^{A1}-L^{A1}-L^{A4}-_{bb};
L^{A4}-C(═O)-L^{A4}-_{bb}; and
L^{A4}-L^{A1}-L^{A4}-C(═O)-_{bb},
wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 5. The compound of any one of Embodiments 1-4, wherein each L^{A4} is independently a monocyclic 4-6 membered nitrogen-containing heterocyclylene optionally substituted with 1-3 R^{a},
wherein each L^{A4} contains 1-2 ring nitrogen atoms and no additional ring heteroatoms.

Embodiment 6. The compound of any one of Embodiments 1-4, wherein one L^{A4} is a monocyclic 4-6 membered nitrogen-containing heterocyclylene optionally substituted with 1-3 R^{a}; and the other L^{A4} is a bicyclic spirocyclic 6-12, optionally (e.g., 6-10) membered nitrogen-containing heterocyclylene optionally substituted with 1-3 R^{a},
wherein each L^{A4} contains 1-2 ring nitrogen atoms and no additional ring heteroatoms.

Embodiment 7. The compound of any one of Embodiments 1-6, wherein L is -L^{A4}-L^{A1}-L^{A4}-_{bb}; and L^{A}N is CH₂ or CHMe.

Embodiment 8. The compound of any one of Embodiments 1-4, wherein L is selected from the group consisting of the moieties delineated in Table L-I-a:

TABLE L-I-a

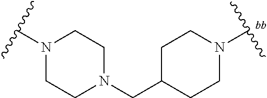

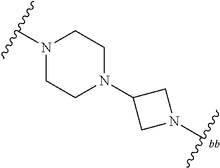

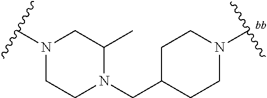

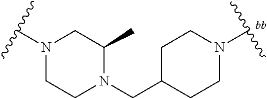

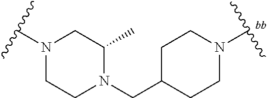

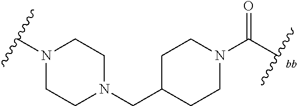

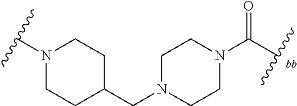

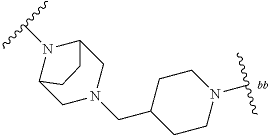

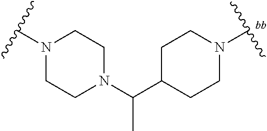

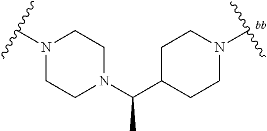

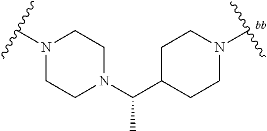

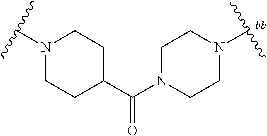

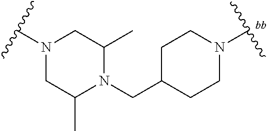

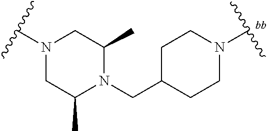

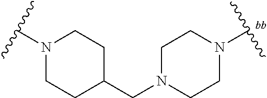

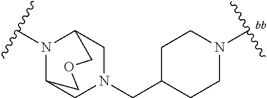

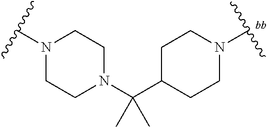

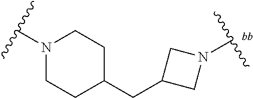

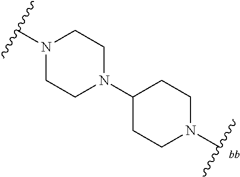

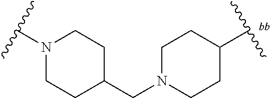

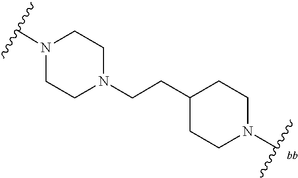

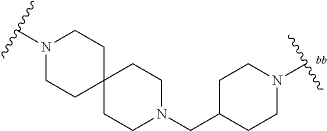

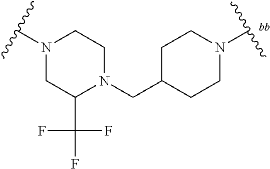

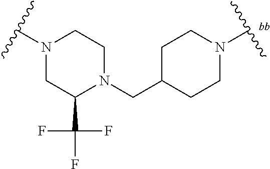

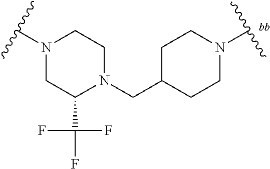

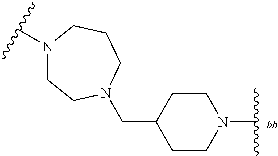

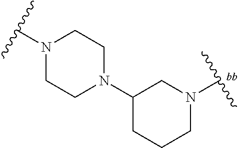

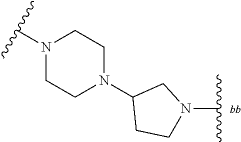

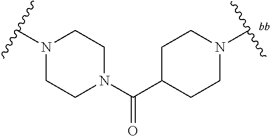

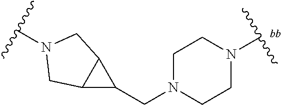

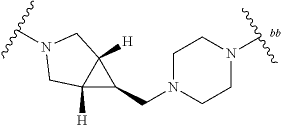

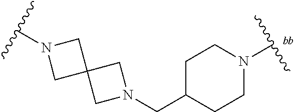

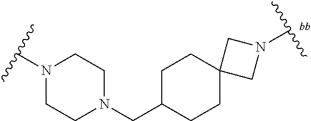

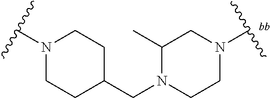

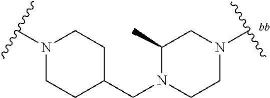

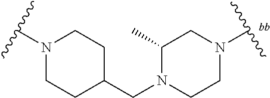

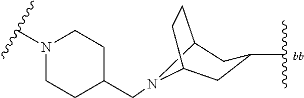

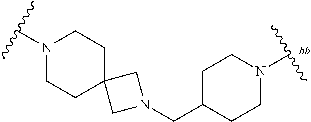

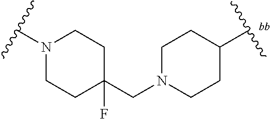

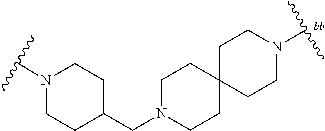

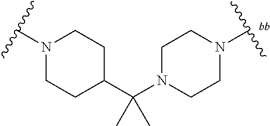

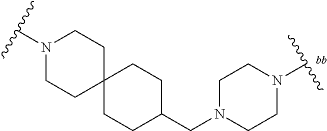

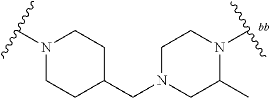

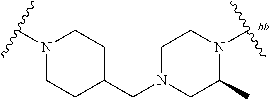

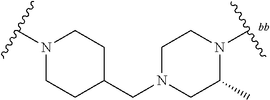

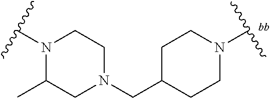

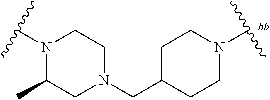

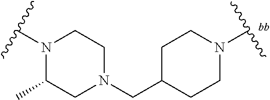

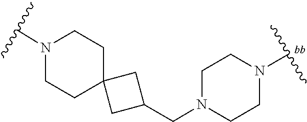

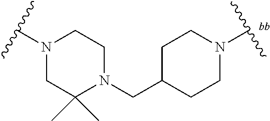

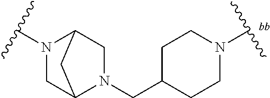

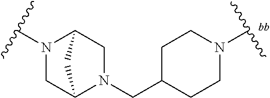

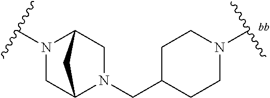

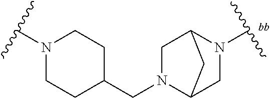

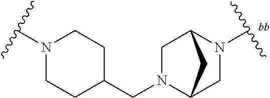

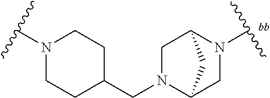

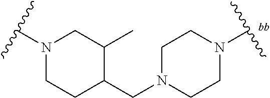

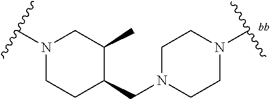

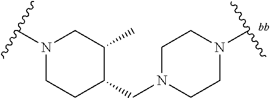

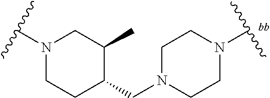

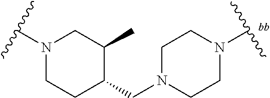

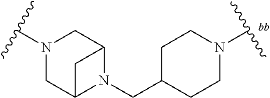

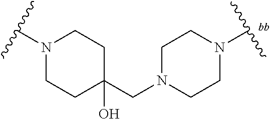

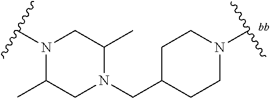

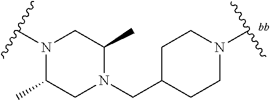

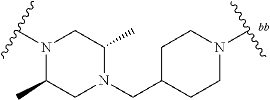

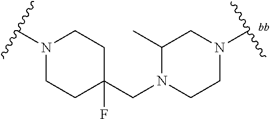

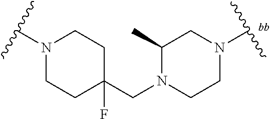

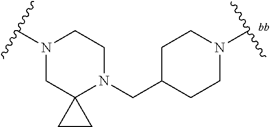

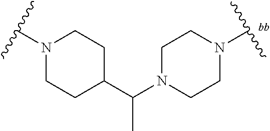

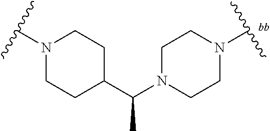

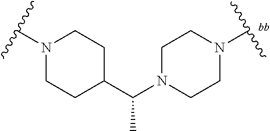

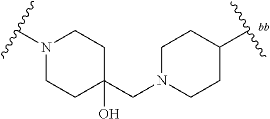

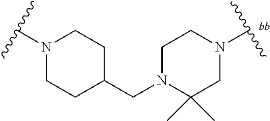

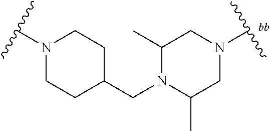

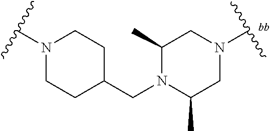

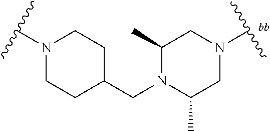

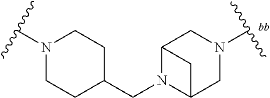

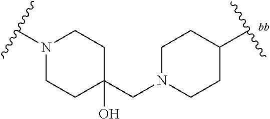

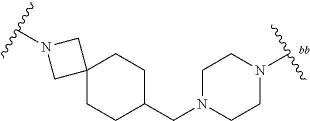

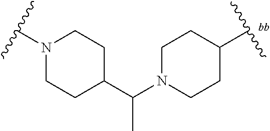

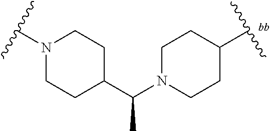

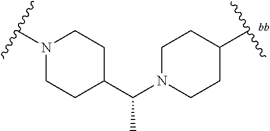

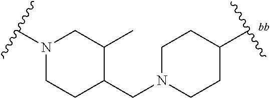

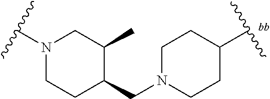

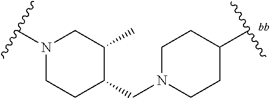

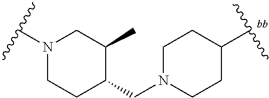

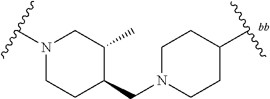

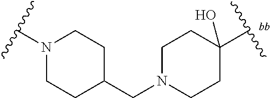

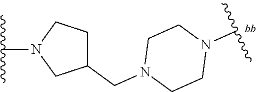

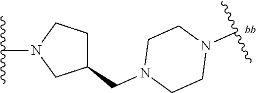

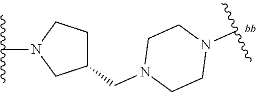

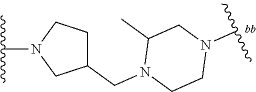

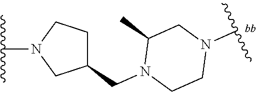

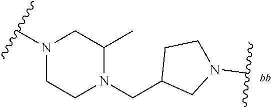

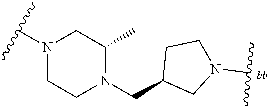

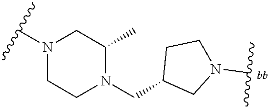

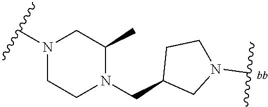

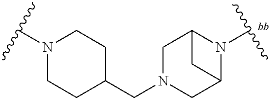

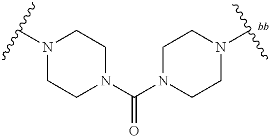

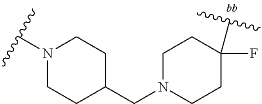

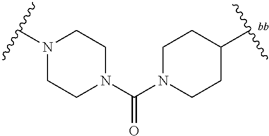

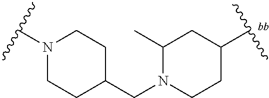

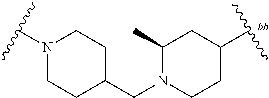

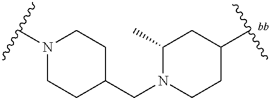

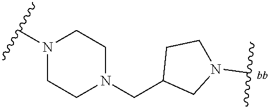

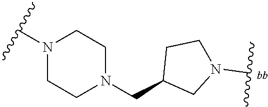

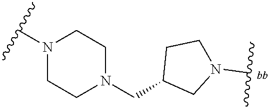

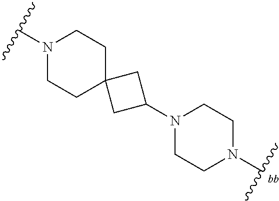

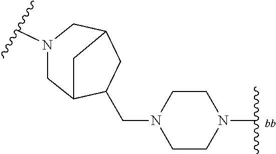

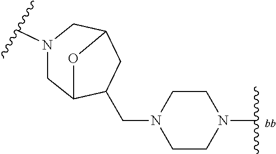

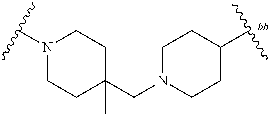

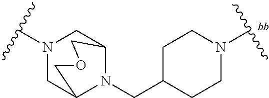

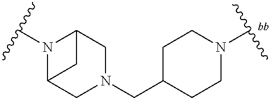

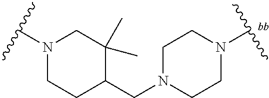

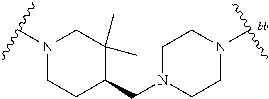

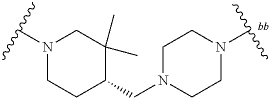

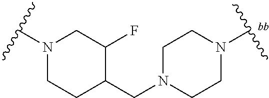

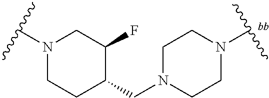

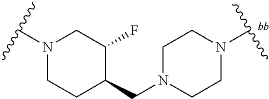

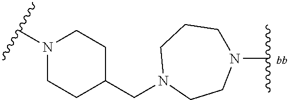

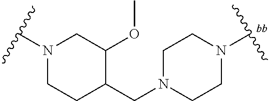

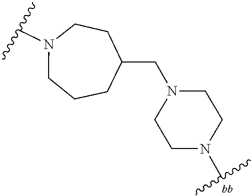

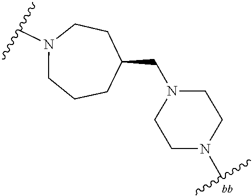

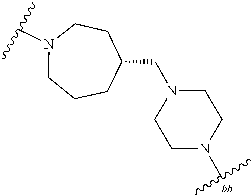

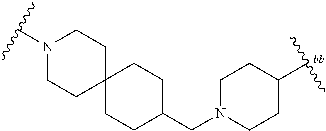

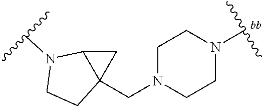

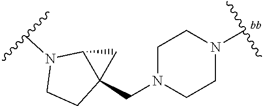

wherein bb represents the point of attachment to Ring C; or

TABLE L-I-a

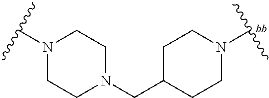

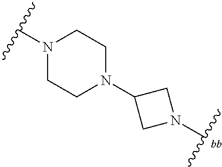

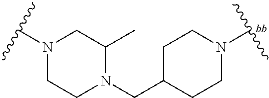

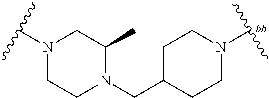

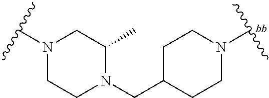

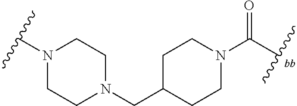

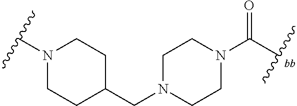

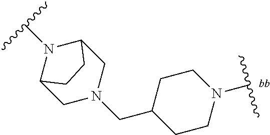

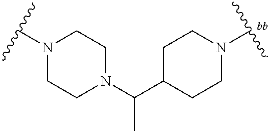

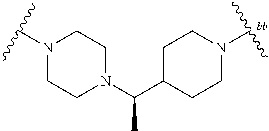

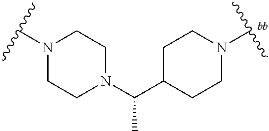

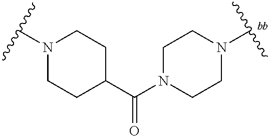

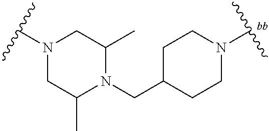

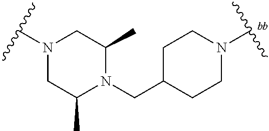

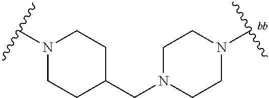

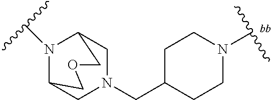

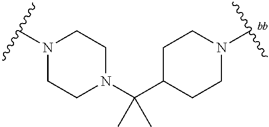

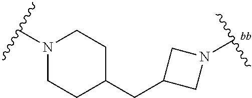

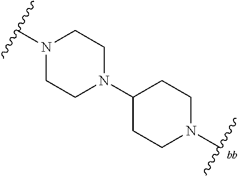

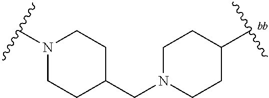

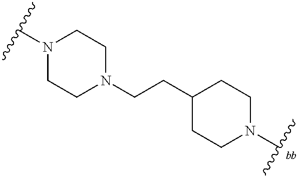

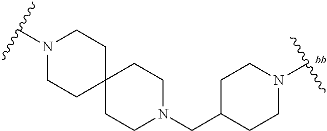

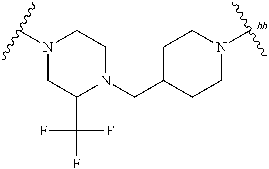

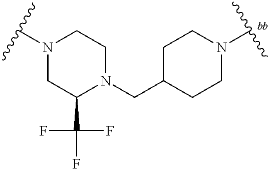

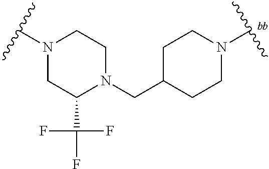

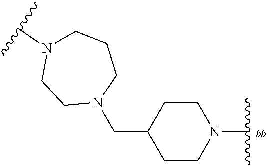

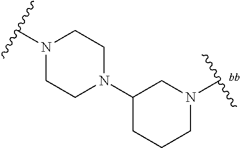

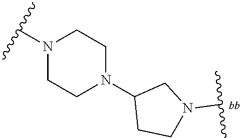

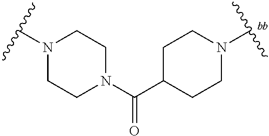

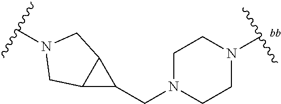

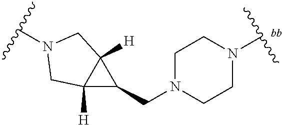

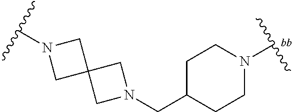

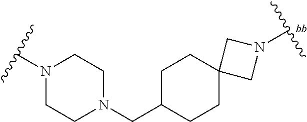

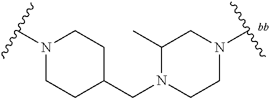

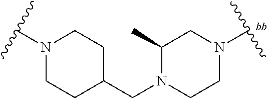

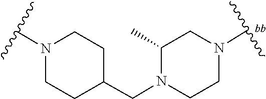

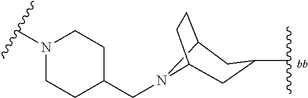

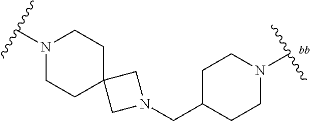

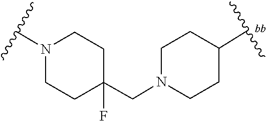

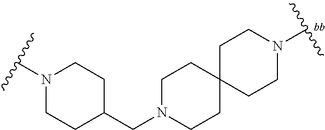

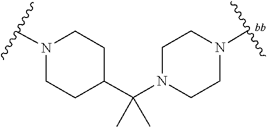

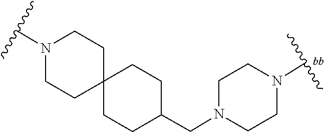

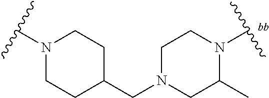

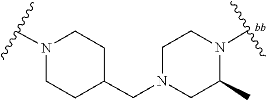

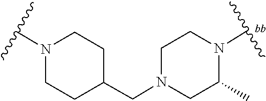

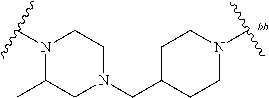

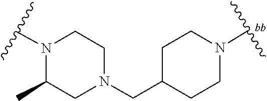

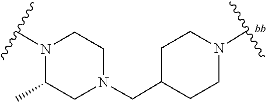

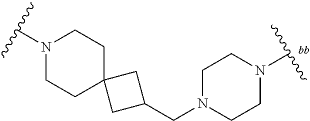

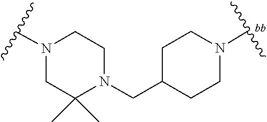

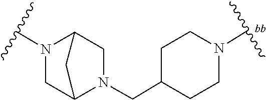

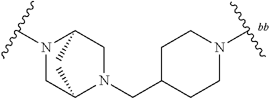

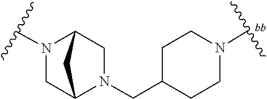

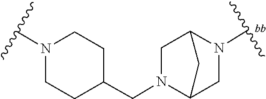

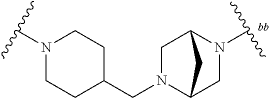

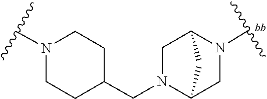

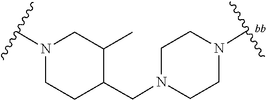

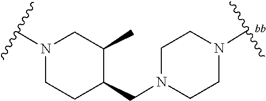

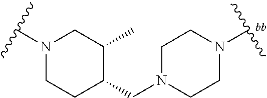

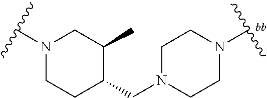

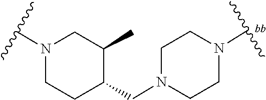

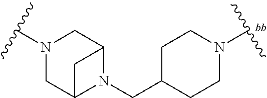

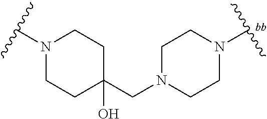

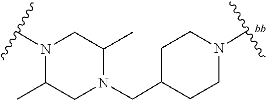

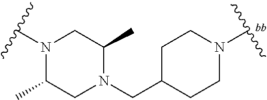

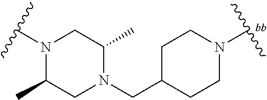

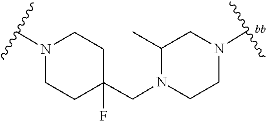

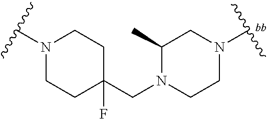

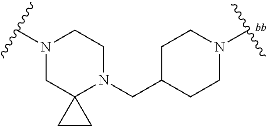

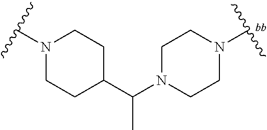

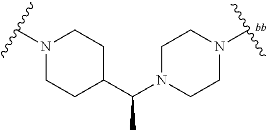

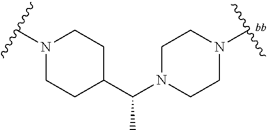

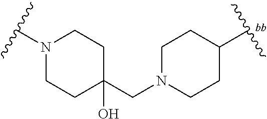

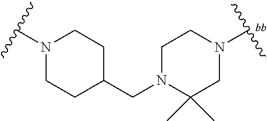

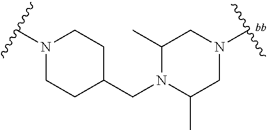

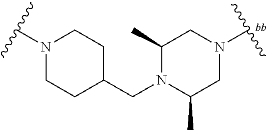

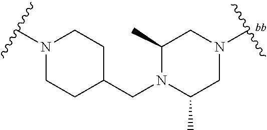

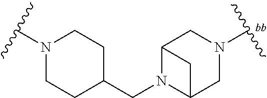

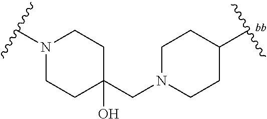

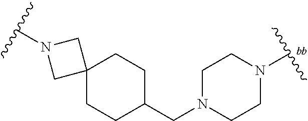

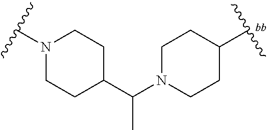

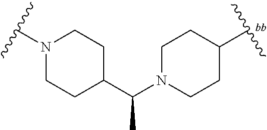

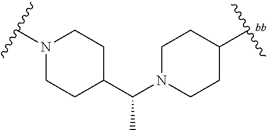

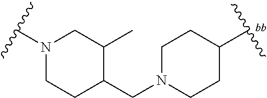

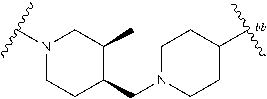

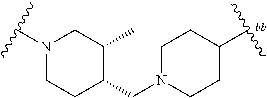

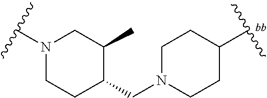

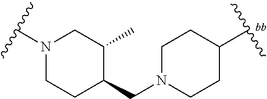

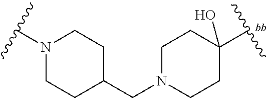

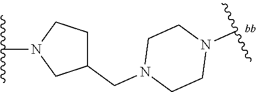

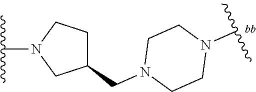

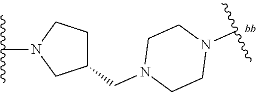

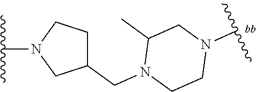

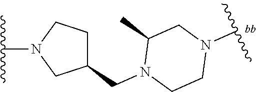

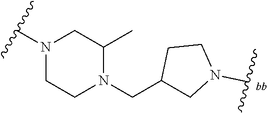

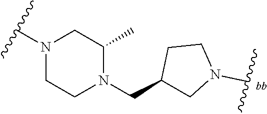

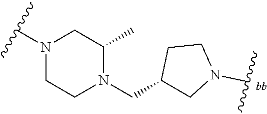

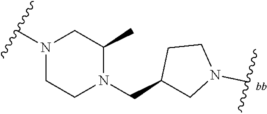

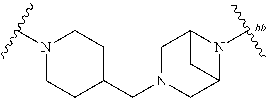

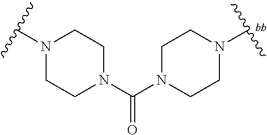

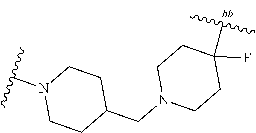

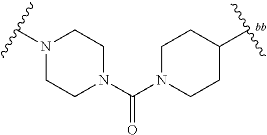

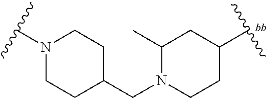

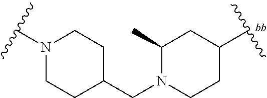

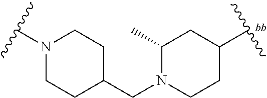

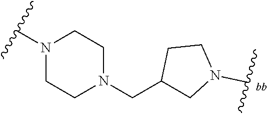

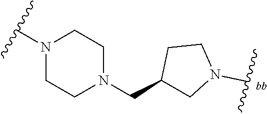

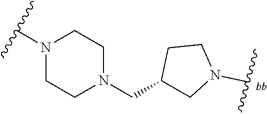

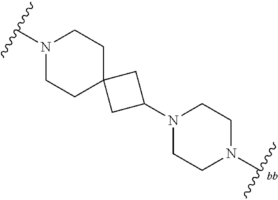

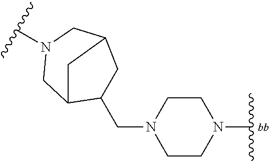

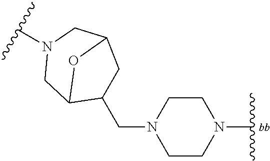

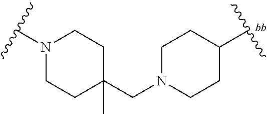

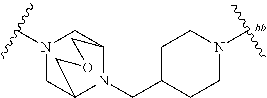

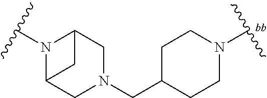

wherein bb represents the point of attachment to Ring C; or

TABLE L1-a

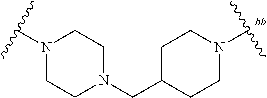

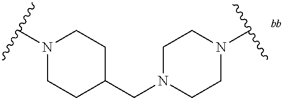

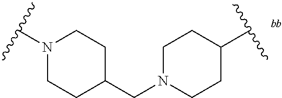

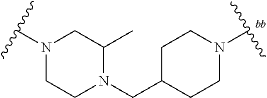

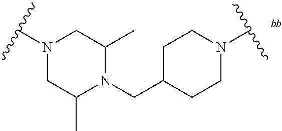

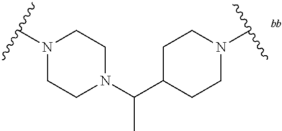

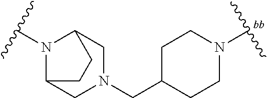

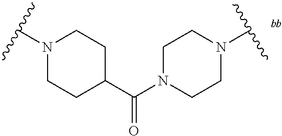

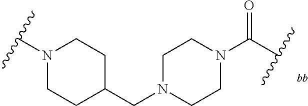

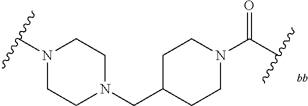

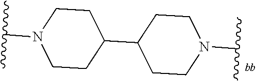

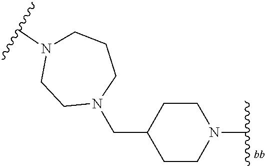

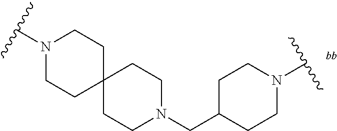

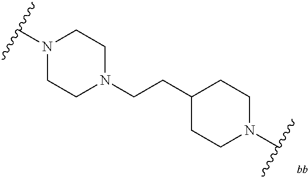

wherein bb represents the point of attachment to Ring C.

Embodiment 9. The compound of Embodiment 1, wherein the compound is a compound of Formula (T-aa-1):

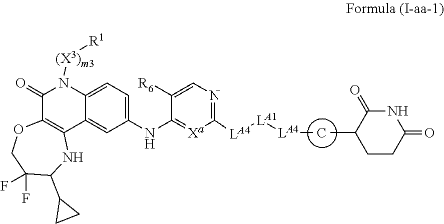

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

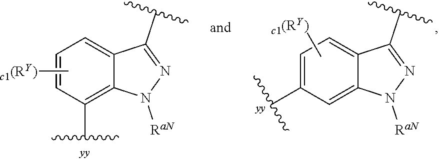

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

Embodiment 10. The compound of any one of Embodiments 1-4 or 9, wherein the compound is a compound of Formula (I-a-1):

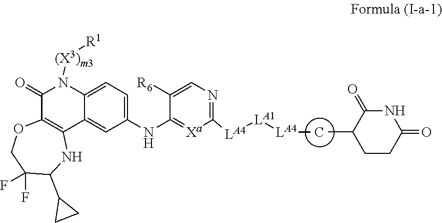

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

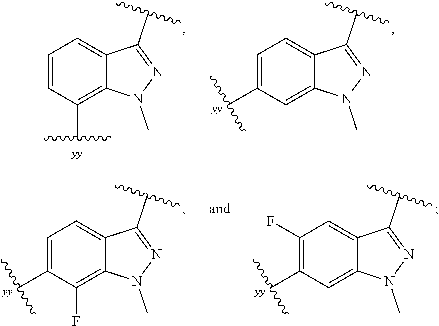 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

Embodiment 11. The compound of Embodiments 9 or 10, wherein the -L^{A4}-L^{A1}-L^{A4}-moiety is selected from the group consisting of the moieties depicted in Table L-I-a-1:

TABLE L-I-a-1

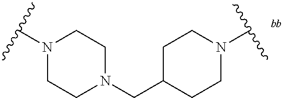

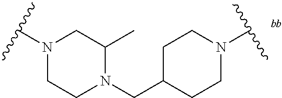

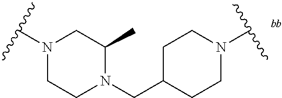

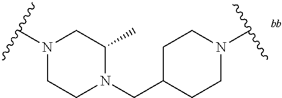

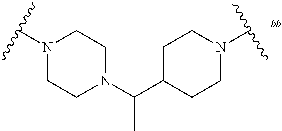

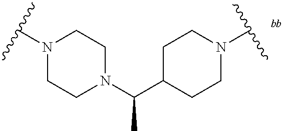

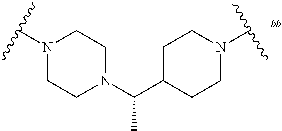

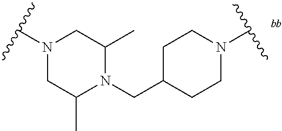

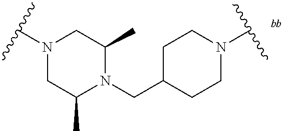

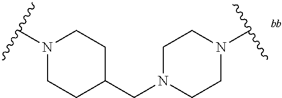

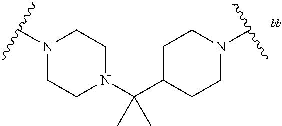

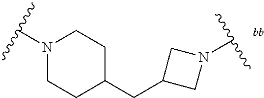

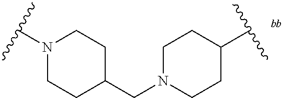

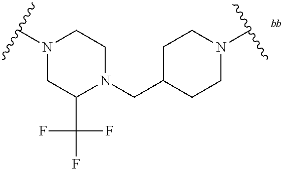

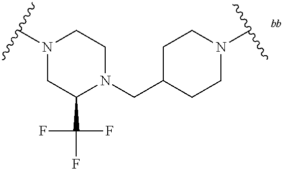

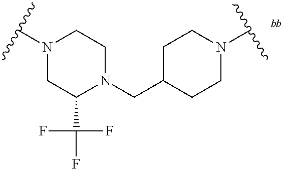

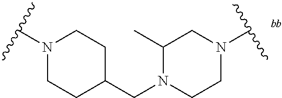

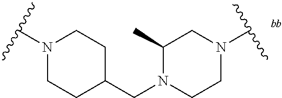

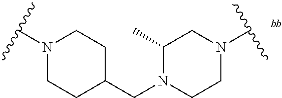

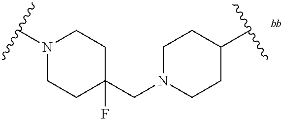

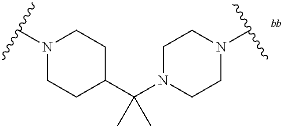

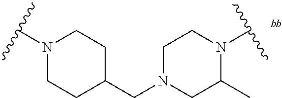

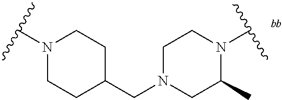

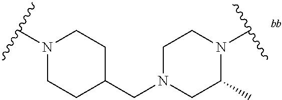

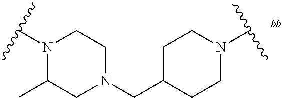

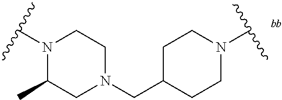

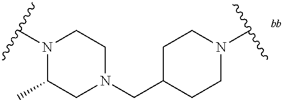

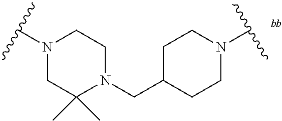

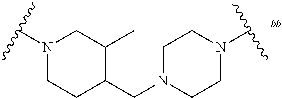

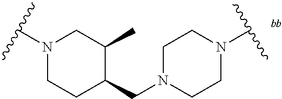

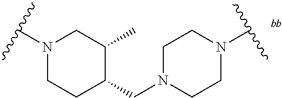

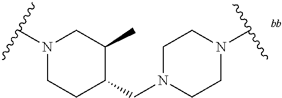

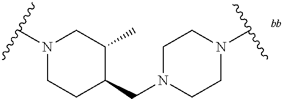

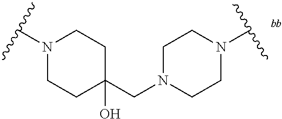

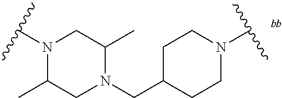

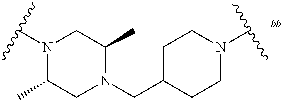

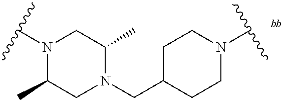

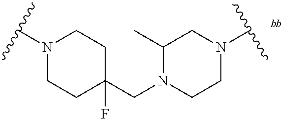

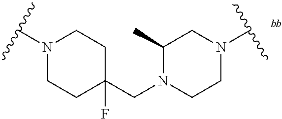

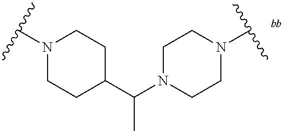

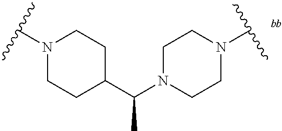

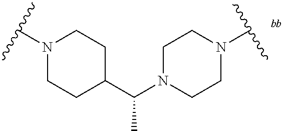

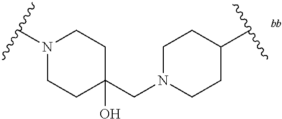

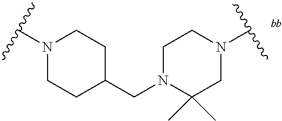

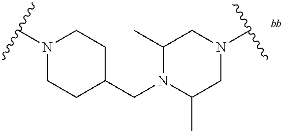

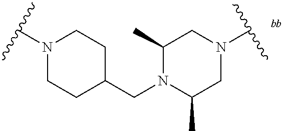

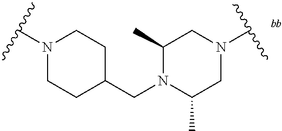

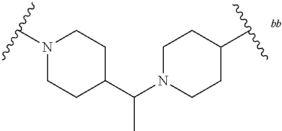

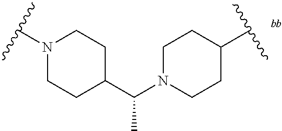

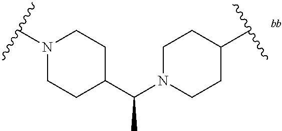

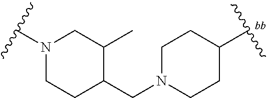

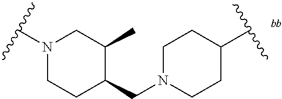

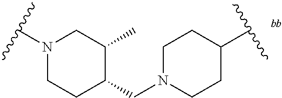

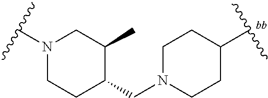

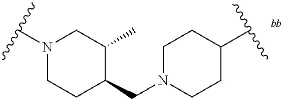

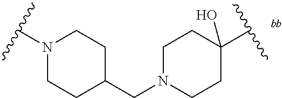

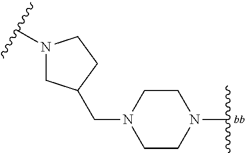

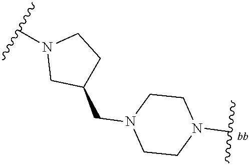

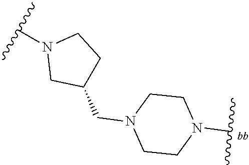

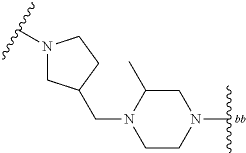

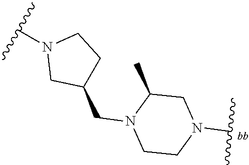

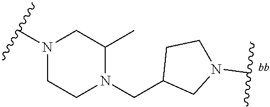

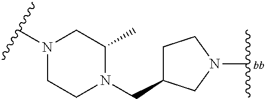

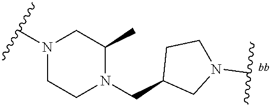

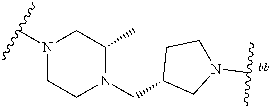

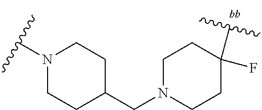

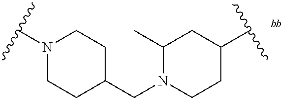

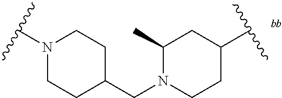

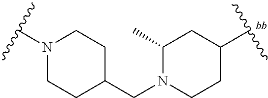

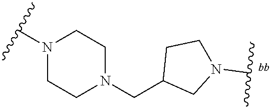

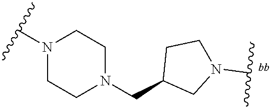

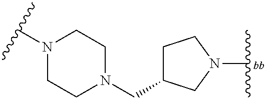

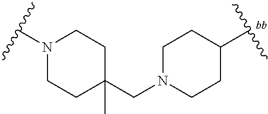

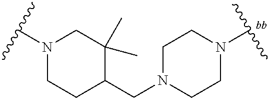

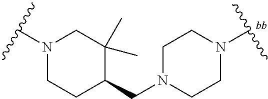

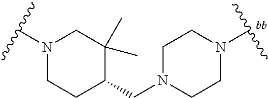

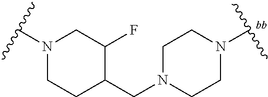

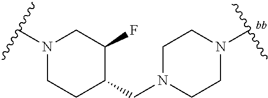

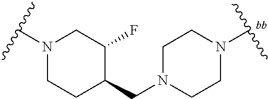

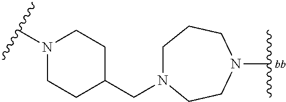

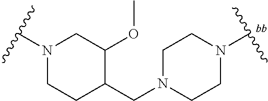

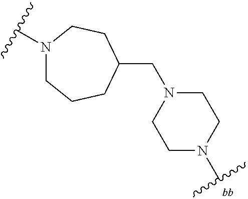

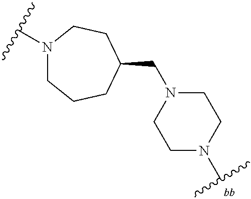

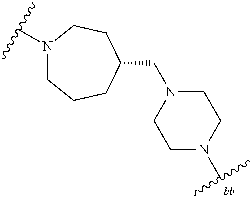

wherein bb represents the point of attachment to Ring C; or

TABLE L-I-a-1

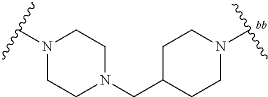

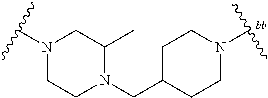

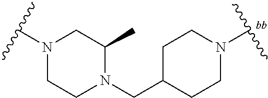

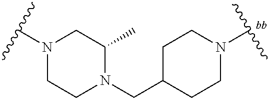

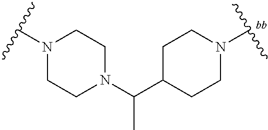

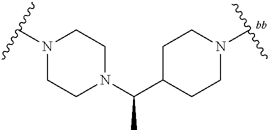

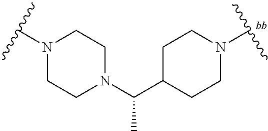

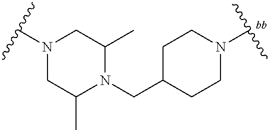

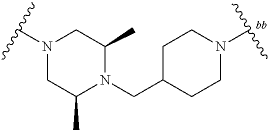

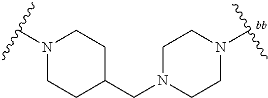

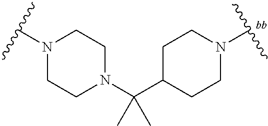

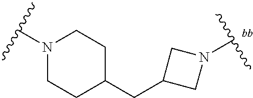

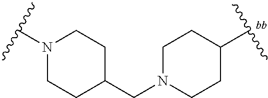

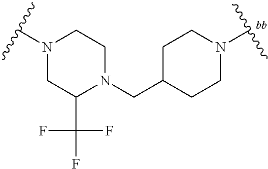

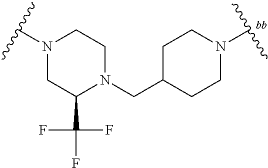

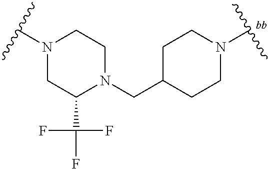

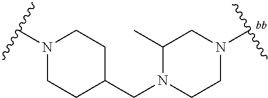

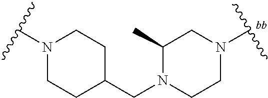

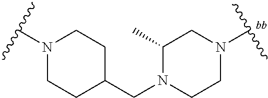

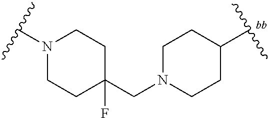

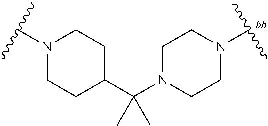

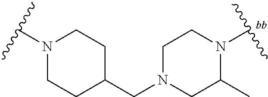

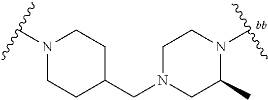

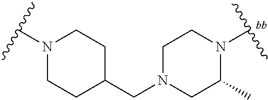

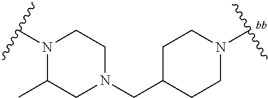

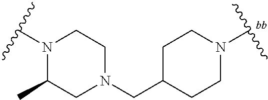

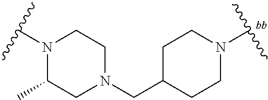

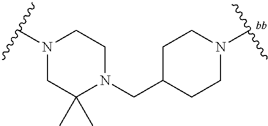

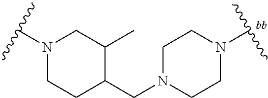

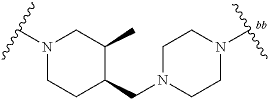

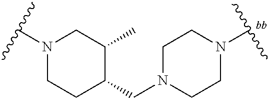

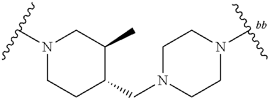

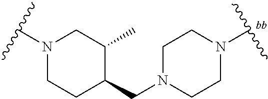

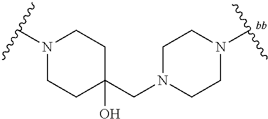

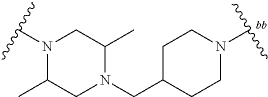

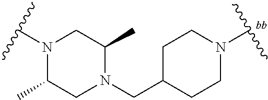

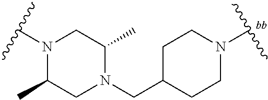

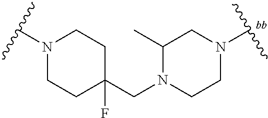

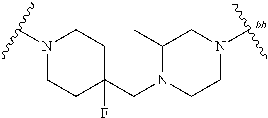

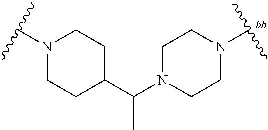

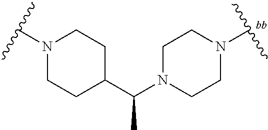

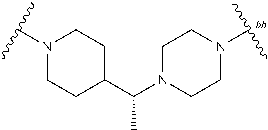

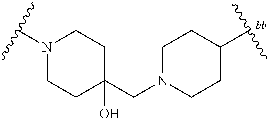

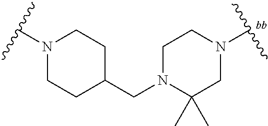

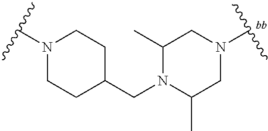

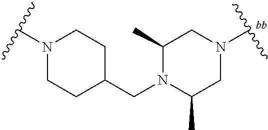

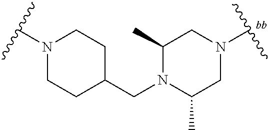

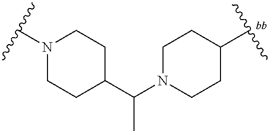

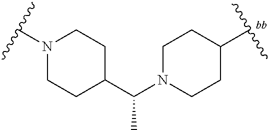

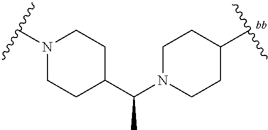

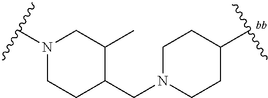

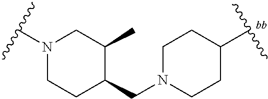

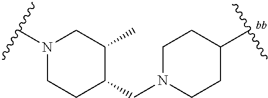

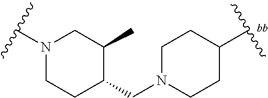

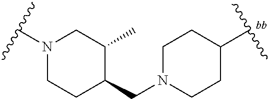

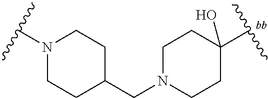

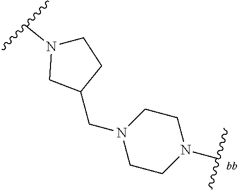

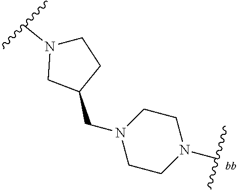

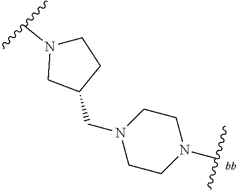

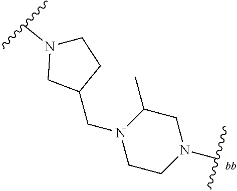

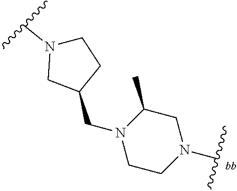

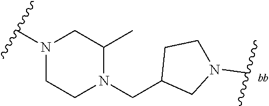

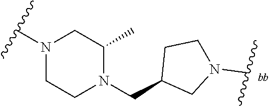

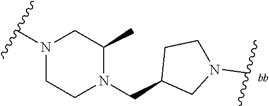

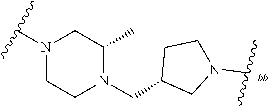

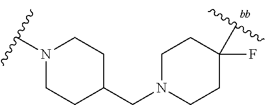

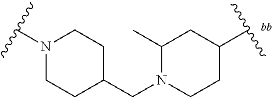

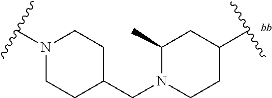

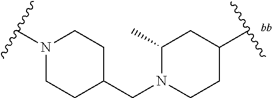

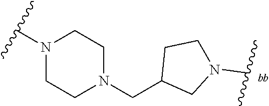

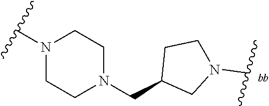

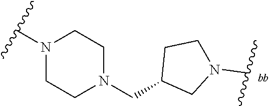

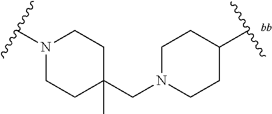

wherein bb represents the point of attachment to Ring C.

Embodiment 12. The compound of Embodiment 1 or 9, wherein the compound is a compound of Formula (I-aa-2):

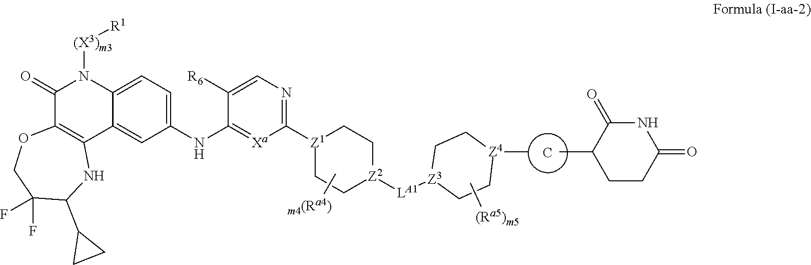

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

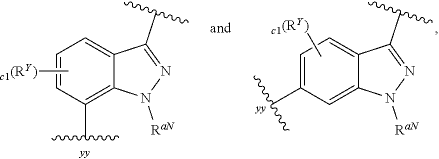

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F.

Embodiment 13. The compound of any one of Embodiments 1-4, 9, 10, or 12, wherein the compound is a compound of Formula (I-a-2):

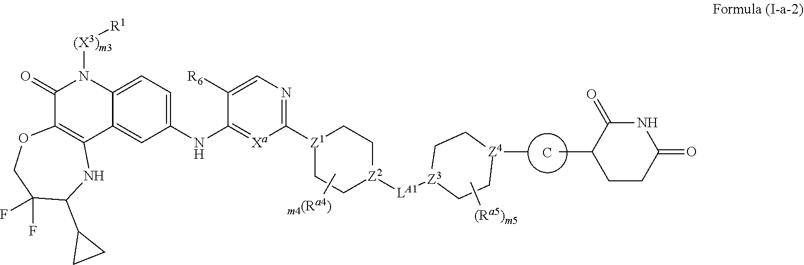

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

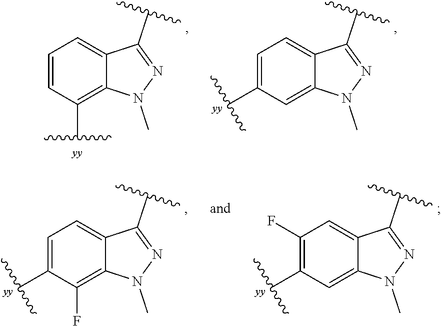 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F.

Embodiment 14. The compound of Embodiments 12 or 13, wherein Z¹ is N; and Z² is CH or CR^{a4}

Embodiment 15. The compound of Embodiments 12 or 13, wherein Z¹ is N; Z² is CH or CR¹⁴; and Z³ is N.

Embodiment 16. The compound of Embodiments 12 or 13, wherein the

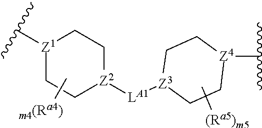

moiety is selected from the group consisting of the moieties depicted in Table L-I-a-2:

TABLE L-I-a-2

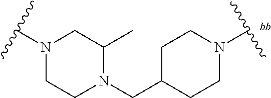

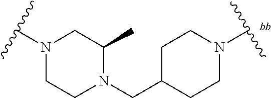

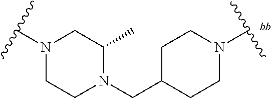

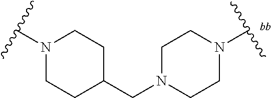

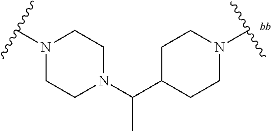

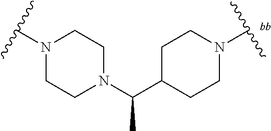

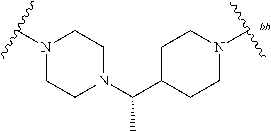

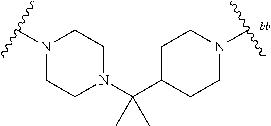

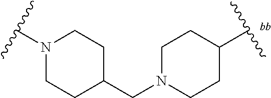

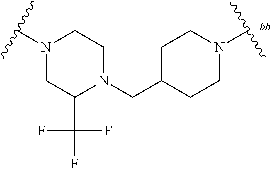

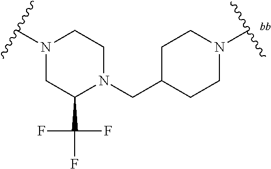

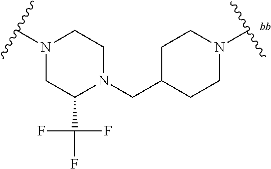

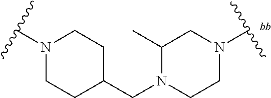

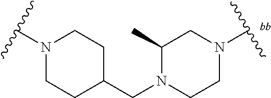

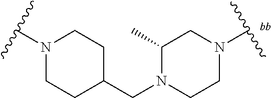

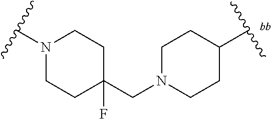

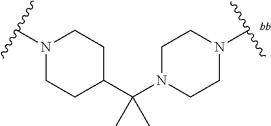

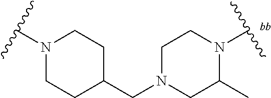

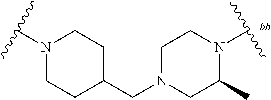

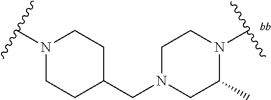

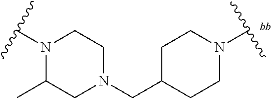

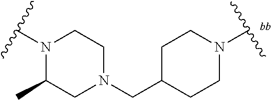

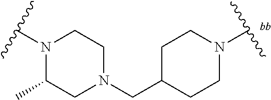

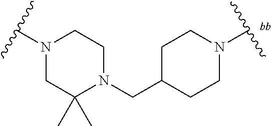

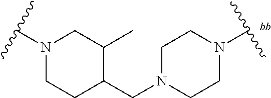

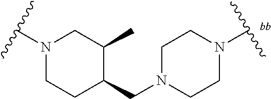

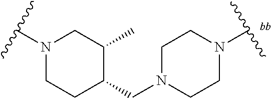

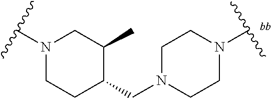

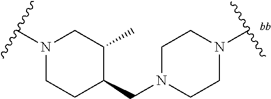

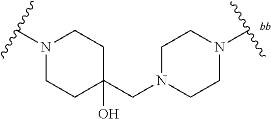

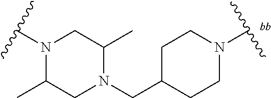

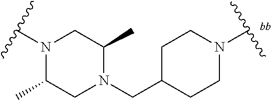

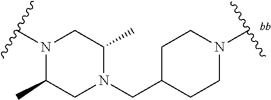

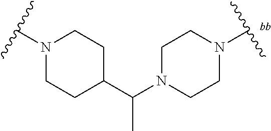

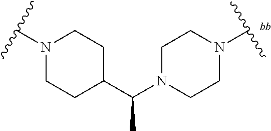

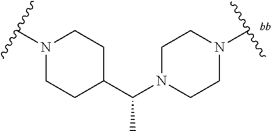

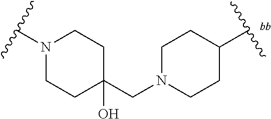

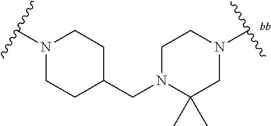

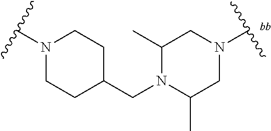

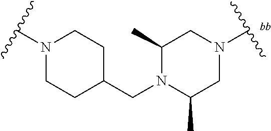

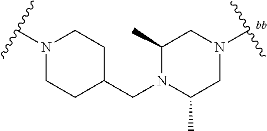

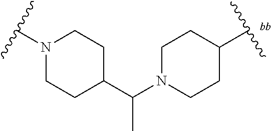

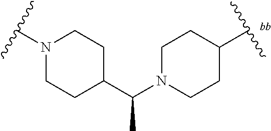

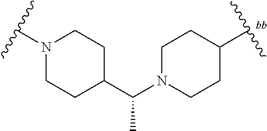

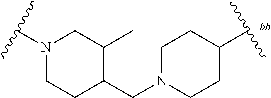

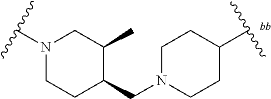

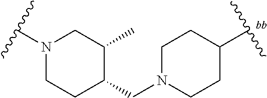

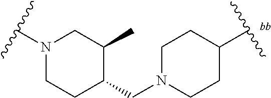

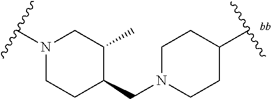

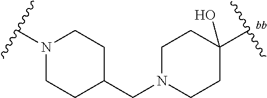

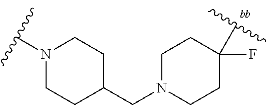

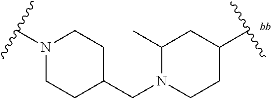

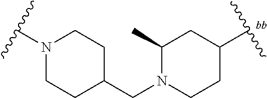

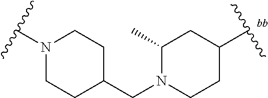

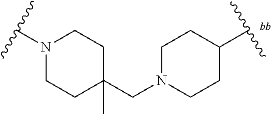

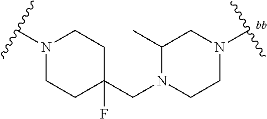

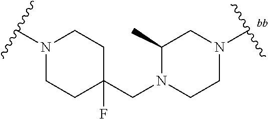

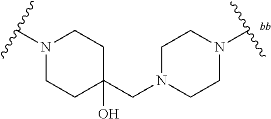

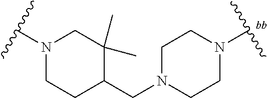

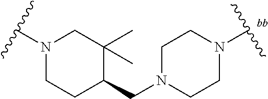

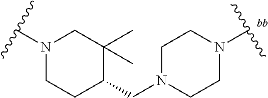

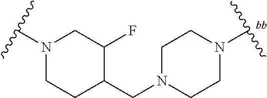

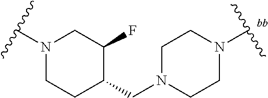

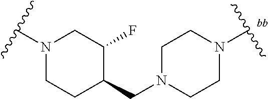

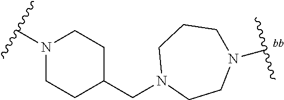

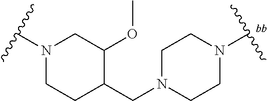

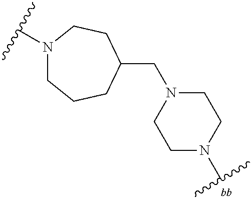

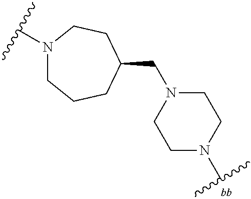

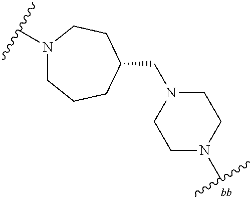

wherein bb represents the point of attachment to Ring C; or

TABLE L-I-a-2

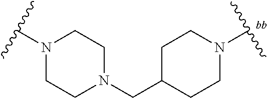

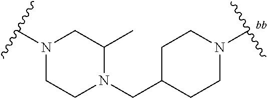

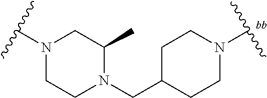

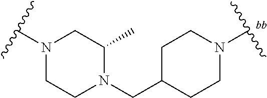

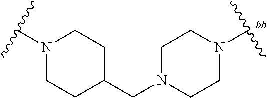

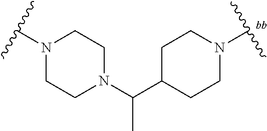

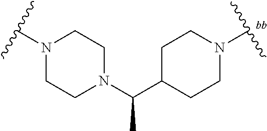

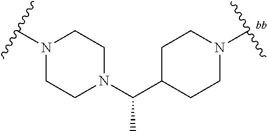

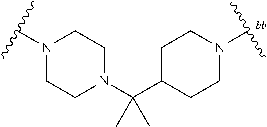

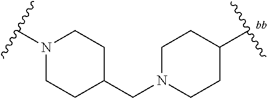

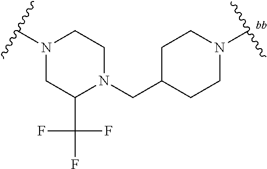

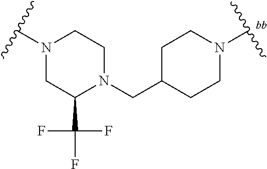

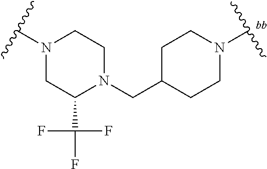

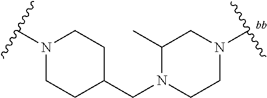

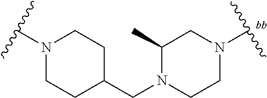

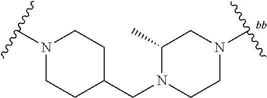

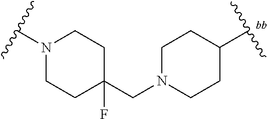

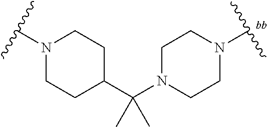

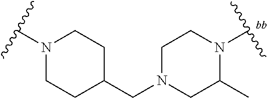

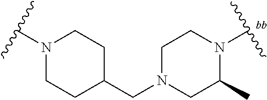

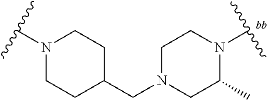

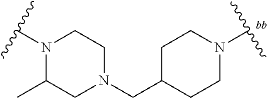

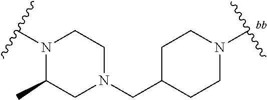

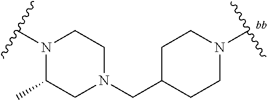

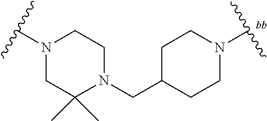

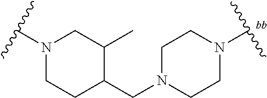

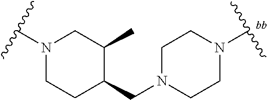

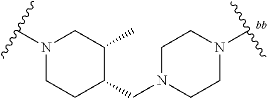

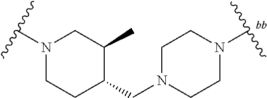

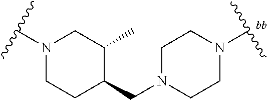

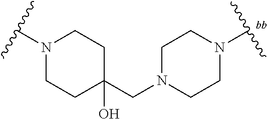

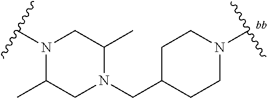

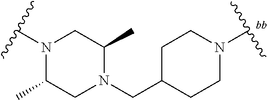

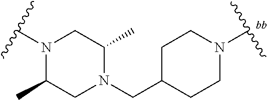

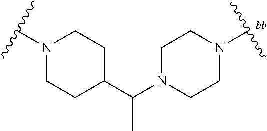

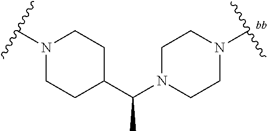

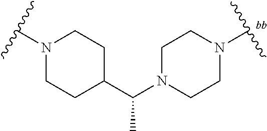

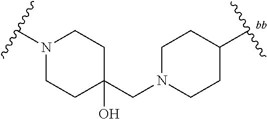

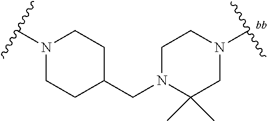

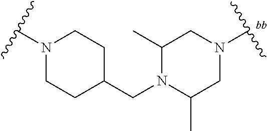

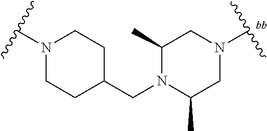

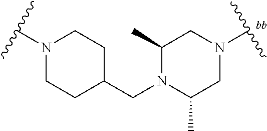

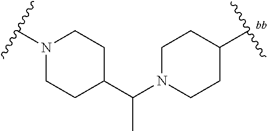

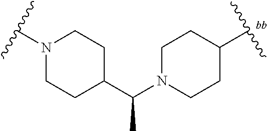

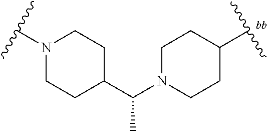

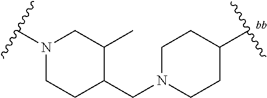

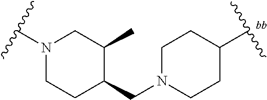

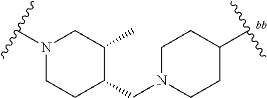

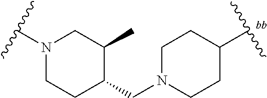

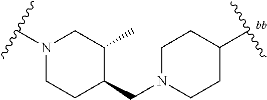

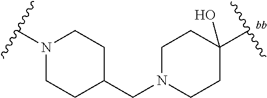

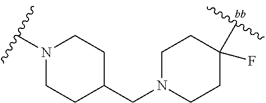

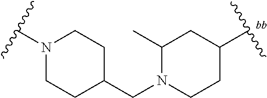

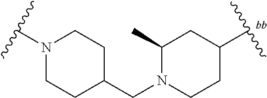

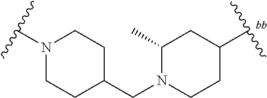

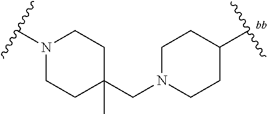

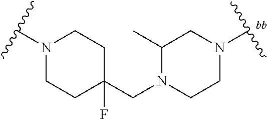

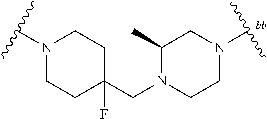

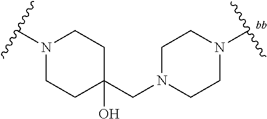

wherein bb represents the point of attachment to Ring C.

Embodiment 17. The compound of Embodiment 1, wherein the compound is a compound of Formula (I-aa-3):

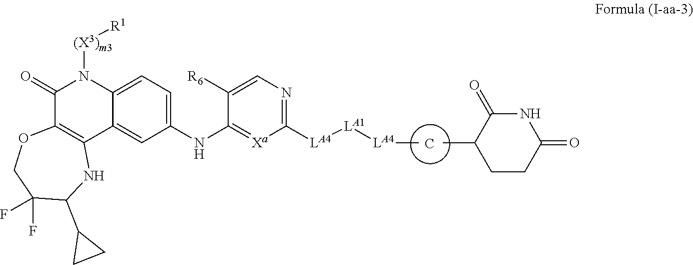

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

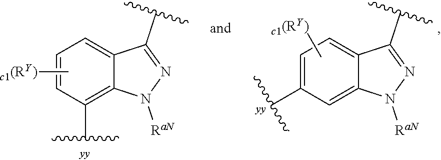

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 18. The compound of any one of Embodiments 1-4 or 17, wherein the compound is a compound of Formula (I-a-3):

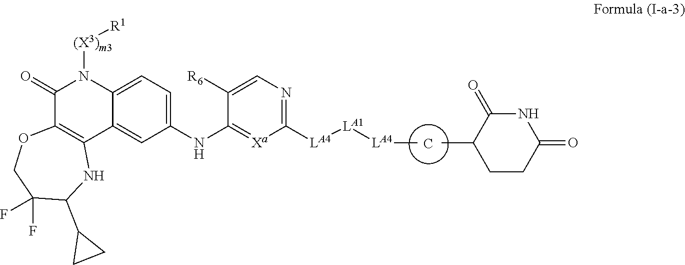

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

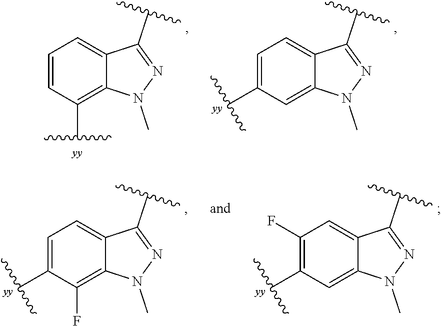 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂;
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 19. The compound of Embodiments 17 or 18, wherein one L^{A4} is a monocyclic 4-6 membered nitrogen-containing heterocyclylene optionally substituted with 1-3 R^{a};
the other L^{A4} is a bicyclic spirocyclic 6-12 (e.g., 7, 9, or 11) membered nitrogen-containing heterocyclylene optionally substituted with 1-3 R^{a}; and
each L^{A4} contains 1-2 ring nitrogen atoms and no additional ring heteroatom.

Embodiment 20. The compound of Embodiments 17 or 18, wherein one L^{A4} is a monocyclic 4-6 membered nitrogen-containing heterocyclylene optionally substituted with 1-3 R^{a};
the other L^{A4} is a bicyclic bridged 6-12 (e.g., 7, 8, or 9) membered nitrogen-containing heterocyclylene optionally substituted with 1-3 R^{a}; and
each L^{A4} contains 1-2 ring nitrogen atoms and no additional ring heteroatom.

Embodiment 21. The compound of Embodiments 17 or 18, wherein one L^{A4} is a monocyclic 4-6 membered nitrogen-containing heterocyclylene optionally substituted with 1-3 R^{a}; the other L^{A4} is a bicyclic fused 6-12 (e.g., 6) membered nitrogen-containing heterocyclylene optionally substituted with 1-3 R^{a}; and
each L^{A4} contains 1-2 ring nitrogen atoms and no additional ring heteroatom.

Embodiment 22. The compound of Embodiments 17 or 18, wherein the -L^{A4}-L^{A1}-L^{A4}-moiety is selected from the group consisting of the moieties depicted in Table L-I-a-3:

TABLE L-I-a-3

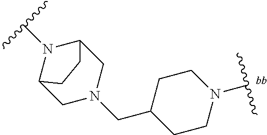

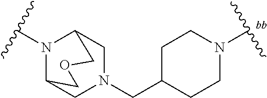

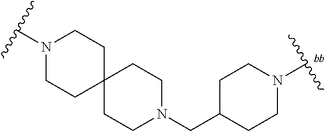

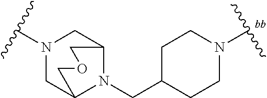

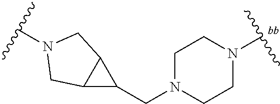

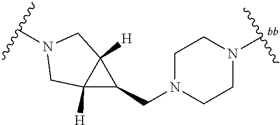

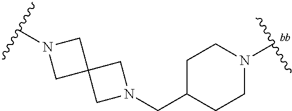

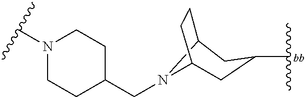

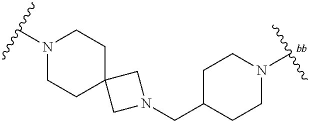

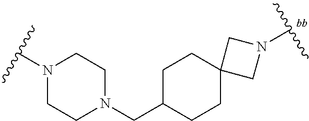

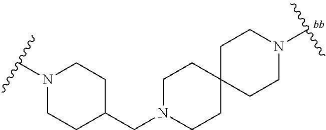

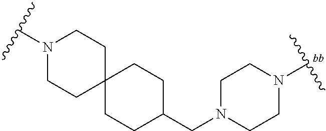

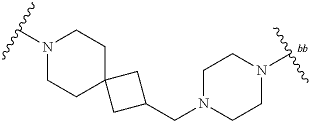

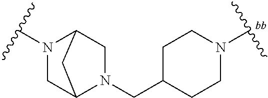

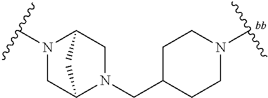

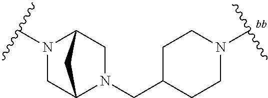

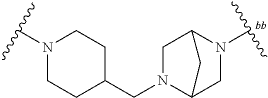

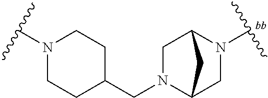

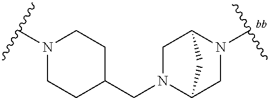

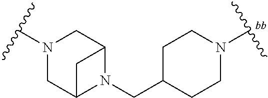

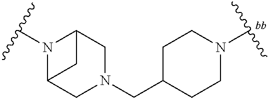

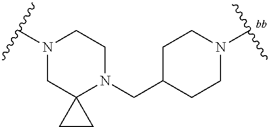

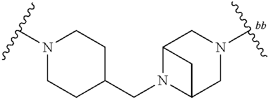

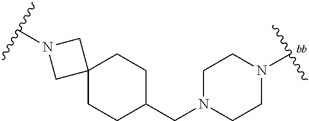

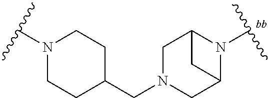

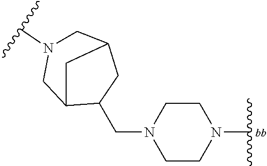

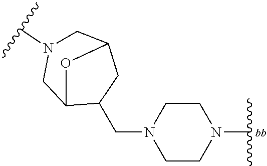

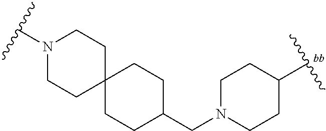

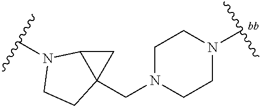

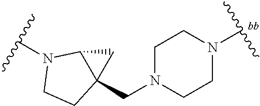

wherein bb represent the point of attachment to Ring C; or

TABLE L-I-a-3

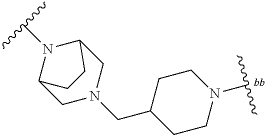

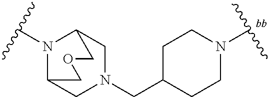

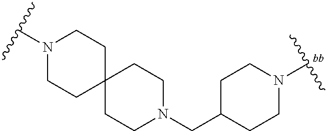

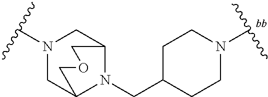

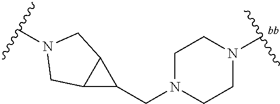

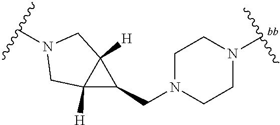

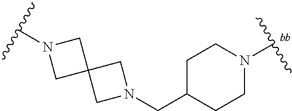

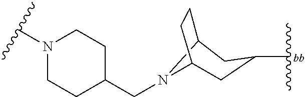

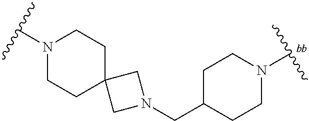

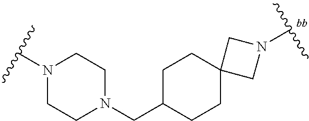

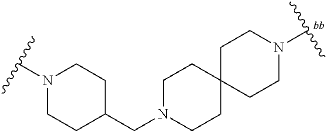

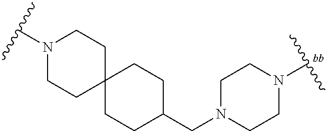

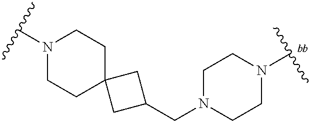

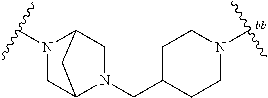

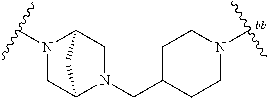

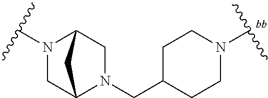

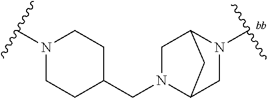

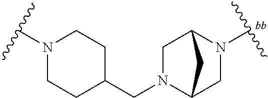

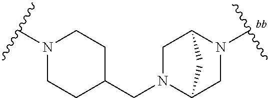

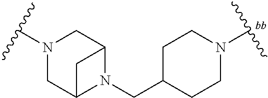

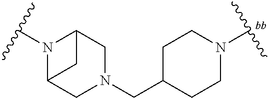

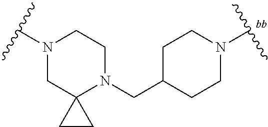

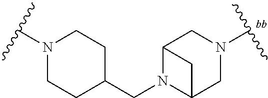

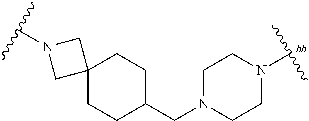

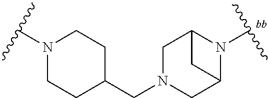

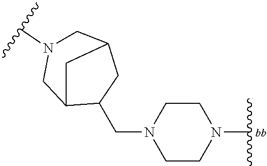

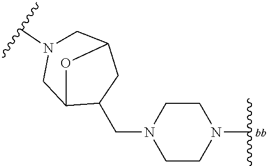

wherein bb represents the point of attachment to Ring C.

Embodiment 23. The compound of any one of Embodiments 1 or 17, wherein the compound is a compound of Formula (I-aa-4):

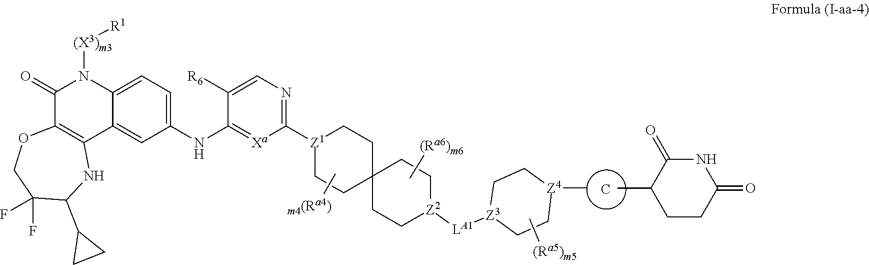

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

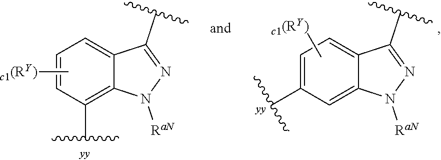

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

Embodiment 24. The compound of any one of Embodiments 1-4, 17, 18, or 23, wherein the compound is a compound of Formula (I-a-4):

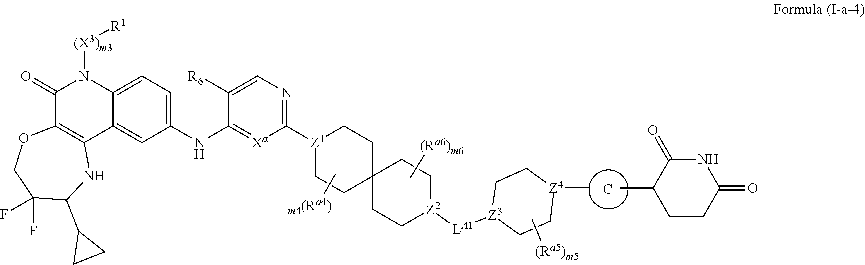

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

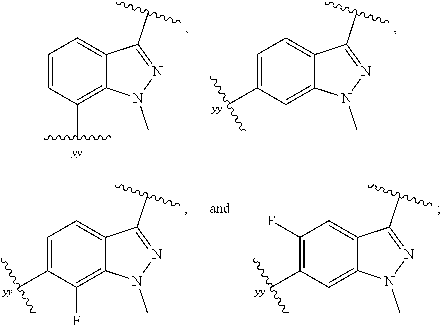 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

Embodiment 25. The compound of Embodiments 23 or 24, wherein the

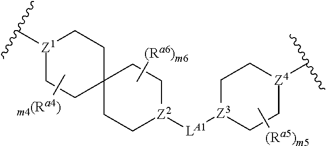

moiety is selected from the group consisting of the moieties depicted in Table L-I-a-4

TABLE L-I-a-4

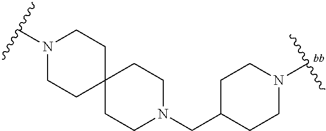

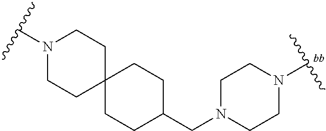

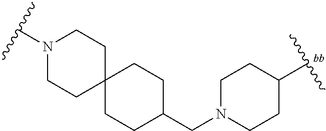

wherein bb represents the point of attachment to Ring C.

TABLE L-I-a-4

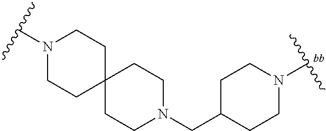

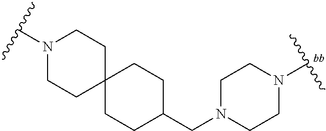

wherein bb represents the point of attachment to Ring C.

Embodiment 26. The compound of any one of Embodiments 1-25, wherein X^{a} is N.

Embodiment 27. The compound of any one of Embodiments 1-25, wherein X^{a} is CH.

Embodiment 28. The compound of Embodiment 1 or 2, wherein the compound is selected from the group consisting of Compound No. 101, 101a, 102, 102a, 130, 130a, 132, 132a, 133, 133a, 186, 186a, 214, 214a, 214b, 379, 379a, 379b, 380, 380a, 380b, 381, 381a, 381b, 382, 382a, 383, 383a, 385, 385a, 386, 386a, 388, 388a, 391, 391a, 392, 392a, 393, 393a, 394, 394a, 395, 395a, 397, 397a, 398, 398a, 400, 400a, 403, 403a, 404, 404a, 405, 405a, 410, 410a, 411, 411a, 412, 412a, 414, 414a, 415, 415a, 416, 416a, 419, 419a, 420, 420a, 421, 421a, 422, 422a, 425, 425a, 426, 426a, 426b, 427, 427a, 428, 428a, 429, 429a, 430, 430a, 431, 431a, 432, 432a, 434, 434a, 435, 435a, 438, 438a, 440, 440a, 442, 442a, 442b, 443, 443a, 443b, 444, 444a, 445, 445a, 446, 446a, 450, 450a, 452, 452a, 453, 453a, 458, 458a, 461, 461a, 462, 462a, 463, 463a, 467, 467a, 469, 469a, 470, 470a, 472, 472a, 473, 473a, 474, 474a, 475, 475a, 475b, 476, 476a, 476b, 477, 477a, 479, 479a, 480, 480a, 481, 481a, 481b, 482, 482a, 482b, 483, 483a, 485, 485a, 486, 486a, 486b, 486c, 486d, 487, 487a, 487b, 487c, 487d, 488, 488a, 489, 489a, 491, 491a, 491b, 493, 493a, 494, 494a, 495, 495a, 496, 496a, 496b, 497, 497a, 498, 498a, 499, 499a, 504, 504a, 505, 505a, 506, 506a, 506b, 507, 507a, 509, 509a, 510, 510a, 513, 513a, 514, 514a, 516, 516a, 521, 521a, 522, 522a, 522b, 523, 523a, 523b, 525, 525a, 525b, 525c, 525d, 526, 526a, 527, 527a, 527b, 528, 528a, 529, 529a, 529b, 529c, 530, 530a, 531, 531a, 532, 532a, 533, 533a, 537, 537a, 538, 538a, 538b, 540, 540a, 541, 541a, 548, 548a, 548b, 550, 550a, 552, 552a, 553, 553a, 554, 554a, 555, 555a, 556, 556a, 557, 557a, 558, 558a, 561, 561a, 563, 563a, 563b, 564, 564a, 565, 565a, 568, 568a, 568b, 569, 569a, 572, 572a, 573, 573a, 574, 574a, 576, 576a, 577, 577a, 578, 578a, 578b, 579, 579a, 579b, 581, 581a, 582, 582a, and 582b as depicted in Table C1, or a pharmaceutically acceptable salt thereof; or
Compound No. 101, 101a, 102, 102a, 130, 130a, 132, 132a, 133, 133a, 186, 186a, 214, 214a, 214b, 379, 379a, 379b, 380, 380a, 380b, 381, 381a, 381b, 382, 382a, 383, 383a, 385, 385a, 386, 386a, 395, 395a, 398, 398a, 400, 400a, 403, 403a, 404, 404a, 405, 405a, 412, 412a, 416, 416a, 419, 419a, 421, 421a, 422, 422a, 426, 426a, 426b, 434, 434a, 435, 435a, 438, 438a, 440, 440a, 442, 442a, 442b, 443, 443a, 443b, 444, 444a, 445, 445a, 446, 446a, 450, 450a, 452, 452a, 453, 453a, 458, 458a, 461, 461a, 462, 462a, 463, 463a, 467, 467a, 469, 469a, 470, 470a, 472, 472a, 473, 473a, 474, 474a, 475, 475a, 475b, 476, 476a, 476b, 477, 477a, 479, 479a, 480, 480a, 481, 481a, 481b, 482, 482a, 482b, 483, 483a, 485, 485a, 486, 486a, 486b, 486c, 486d, 487, 487a, 487b, 487c, 487d, 488, 488a, 489, 489a, 491, 491a, 491b, 493, 493a, 494, 494a, 495, 495a, 496, 496a, 496b, 497, 497a, 498, 498a, 499, 499a, 504, 504a, 505, 505a, 506, 506a, 506b, 507, 507a, 509, 509a, 510, 510a, 513, 513a, 514, 514a, 516, 516a, 521, 521a, 522, 522a, 522b, 523, 523a, 523b, 525, 525a, 525b, 525c, 525d, 526, 526a, 527, 527a, 527b, 528, 528a, 529, 529a, 529b, 529c, 530, 530a, 531, 531a, 533, 533a, 538, 538a, 538b, 548, 548a, 548b 550, 550a, 554, 554a, 555, 555a, 556, 556a, 557, 557a, 558, 558a, 561, 561a, 563, 563a, 563b, 564, 564a, 565, and 565a as depicted in Table C1, or a pharmaceutically acceptable salt thereof; or
Compound No. 101, 101a, 102, 102a, 130, 130a, 132, 132a, 133, 133a, 186, 186a, 214, 214a, 214b, 379, 379a, 379b, 380, 380a, 380b, 381, 381a, 381b, 382, 382a, 383, 383a, 385, 385a, 386, 386a, 395, 395a, 398, 398a, 400, 400a, 403, 403a, 404, 404a, 405, 405a, 412, 412a, 416, 416a, 419, 419a, 421, 421a, 422, 422a, 426, 426a, 426b, 434, 434a, 435, 435a, 438, 438a, 440, 440a, 442, 442a, 442b, 443, 443a, 443b, 444, 444a, 445, 445a, 446, 446a, 450, 450a, 452, 452a, 453, 453a, 458, 458a, 461, 461a, 462, 462a, 463, 463a, 467, 467a, 469, 469a, 470, 470a, 472, 472a, 473, 473a, 474, 474a, 475, 475a, 475b, 476, 476a, 476b, 477, 477a, 479, 479a, 480, 480a, 481, 481a, 481b, 482, 482a, 482b, 483, 483a, 485, 485a, 486, 486a, 486b, 486c, 486d, 487, 487a, 487b, 487c, 487d, 488, 488a, 489, 489a, 491, 491a, 491b, 493, 493a, 494, 494a, 495, 495a, 496, 496a, 496b, 497, 497a, 498, 498a, 499, and 499a, as depicted in Table C1, or a pharmaceutically acceptable salt thereof; or
Compound No. 101, 101a, 102, 102a, 130, 130a, 132, 132a, 214, 214a, 214b, 379, 379a, 379b, 380, 380a, 380b, 381, 381a, 381b, 382, 382a, 383, 383a, 385, 385a, 386, 386a, 395, 395a, 398, 398a, 400, 400a, 403, 403a, 404, 404a, 405, 405a, 412, 412a, 416, and 416a as depicted in Table C1, or a pharmaceutically acceptable salt thereof.

Embodiment 29. A pharmaceutical composition comprising a compound of any one of Embodiments 1-28, or a pharmaceutically acceptable salt thereof, and a pharmaceutically acceptable excipient.

Embodiment 30. A compound of any one of Embodiments 1-28, or a pharmaceutically acceptable salt thereof, or the pharmaceutical composition of Embodiment 29 for use in treatment.

Embodiment 31. A compound of any one of Embodiments 1-28 or a pharmaceutically acceptable salt thereof, or the pharmaceutical composition of Embodiment 29 for use in the treatment of a cancer selected from the group consisting of a hematological cancer, breast cancer, gastrointestinal cancer, brain cancer, and lung cancer.

Embodiment 32. The compound or pharmaceutical composition of Embodiment 31, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML); optionally wherein the hematological cancer is selected from the group consisting of: is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), or chronic myeloid leukemia (CML); optionally wherein the hematological cancer is selected from the group consisting of DLBCL, FL, MCL, BL, PTCL, and ALL (e.g., B-ALL); optionally wherein the hematological cancer is FL or DLBCL; optionally where the hematological cancer is DLBCL.

Embodiment 33. The compound or pharmaceutical composition of Embodiment 30 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof for use in treatment; or an additional therapeutic agent or therapy selected from the group consisting of is a PI3K inhibitor, a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof for use in treatment.

Embodiment 34. The compound or pharmaceutical composition of Embodiment 33 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or an additional therapeutic agent or therapy selected from the group consisting of is a PI3K inhibitor, a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof.

Embodiment 35. A compound of any one of Embodiments 1-28, or a pharmaceutically acceptable salt thereof, or the pharmaceutical composition of Embodiment 29 for use in a method of treating cancer selected from the group consisting of the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML); optionally wherein the cancer is selected from the group consisting of: is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), or chronic myeloid leukemia (CML); optionally wherein the cancer is selected from the group consisting of DLBCL, FL, MCL, BL, PTCL, and ALL (e.g., B-ALL); optionally wherein the hematological cancer is FL or DLBCL; optionally where the cancer is DLBCL, the method comprising administering to a subject a therapeutically effective amount of a compound of Embodiments 1-28, or a pharmaceutically acceptable salt thereof, or the pharmaceutical composition of Embodiment 29.

Embodiment 36. The compound or pharmaceutical composition of Embodiment 35, wherein the method comprises administering to the subject a therapeutically effective amount of an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or an additional therapeutic agent or therapy selected from the group consisting of is a PI3K inhibitor, a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof.

Embodiment 37. The compound or pharmaceutical composition of Embodiment 36, wherein the compound of any one of Embodiments 1-28, or a pharmaceutically acceptable salt thereof, or the pharmaceutical composition of Embodiment 29, and the additional therapeutic agent or therapy are administered simultaneously, separately, or sequentially.

### Exemplary Formula (I) Embodiments

Embodiment 1. A compound of Formula (I):

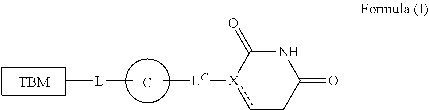

or a pharmaceutically acceptable salt thereof, wherein:
TBM is (T1):

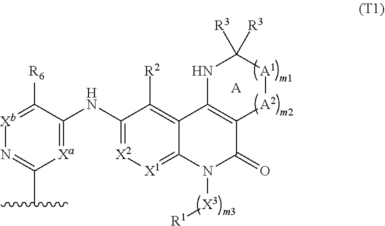

X¹ is selected from the group consisting of N and CR²;
X² is CH;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
m3 is 0 or 1;
X³ is C₁₋₃ alkylene optionally substituted with 1-3 R^{e};
R¹ is selected from the group consisting of: H and C₃₋₆ cycloalkyl;
m1 is 2; each A¹ is independently CH₂, CHR⁴, or CR⁴R⁴;
m2 is 1; A² is —O—;
one R³ is selected from the group consisting of:
(i) C₃₋₆ cycloalkyl optionally substituted with 1-3 R⁹, and
(ii) C₁₋₃ alkyl optionally substituted with 1-3 —F; and
the other R³ is H;
each R⁴ is independently selected from the group consisting of: H, R^{a}, and R^{b};
X^{a} is selected from the group consisting of: N, CH, and CF;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1} L^{A3}, and L^{A4} provided that 1-3 occurrences of L^{A} is L^{A4};

### (BB)

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any 0-0, N—O, N—N, N—S(O)o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{e};
Ring C is selected from the group consisting of:

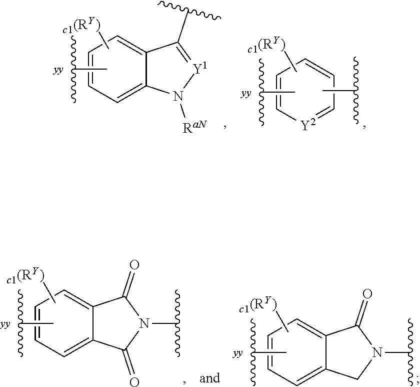

c1 is 0, 1, 2, or 3;
each R¹ is independently selected from the group consisting of: R^{a} and R^{b};
R^{aN} is H or C₁₋₆ alkyl optionally substituted with 1-3 R^{e};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{e};
(f) —NR^{d}R^{e};
(g) C(═O)C₁₋₆ alkyl;
(h) C(═O)C₁₋₆ haloalkyl;
(i) C(═O)OH;
(j) C(═O)OC₁₋₆ alkyl;
(k) C(═O)OC₁₋₆ haloalkyl;
(l) C(═O)N(R^{f})₂;
(m) S(O)₀₋₂(C₁₋₆ alkyl);
(n) S(O)₀₋₂(C₁₋₆ haloalkyl);
(o) S(O)₁₋₂N(R)₂; and
(p) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R⁹ is independently selected from the group consisting of: Rh, oxo, C₁₋₃ alkyl, and C₁₋₃ haloalkyl; and
each R^{h} is independently selected from the group consisting of: halo, cyano, —OH, —(C₀-3 alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

Embodiment 2. The compound of Embodiment 1, wherein Ring C is

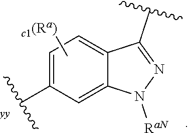

Embodiment 3. The compound of Embodiment 1, wherein Ring C is

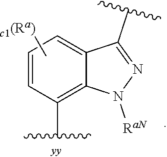

Embodiment 4. The compound of any one of Embodiments 1-3, wherein c1 is 0.

Embodiment 5. The compound of any one of Embodiments 1-3, wherein c1 is 1; and R^{Y} is halo (e.g., —F).

Embodiment 6. The compound of any one of Embodiments 1-5, wherein R^{aN} is C₁₋₃ alkyl (e.g., methyl).

Embodiment 7. The compound of any one of Embodiments 1-6, wherein X is CH.

Embodiment 8. The compound of any one of Embodiments 1-7, wherein L^{C} is a bond.

Embodiment 9. The compound of Embodiment 1, wherein the

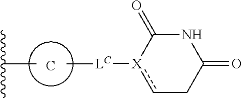

moiety is selected from the group consisting of the moieties delineated in Table CM-1b or Table CM-1a:

TABLE CM-1b

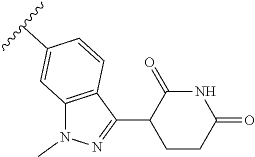 [SMILES: *c1ccc2c(C3CCC(=O)NC3=O)nn(C)c2c1]

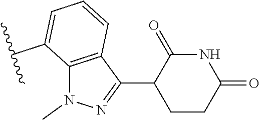 [SMILES: *c1cccc2c(C3CCC(=O)NC3=O)nn(C)c12]

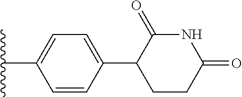 [SMILES: *c1ccc(C2CCC(=O)NC2=O)cc1]

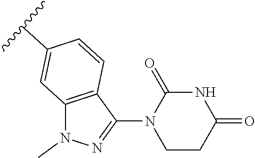 [SMILES: *c1ccc2c(N3CCC(=O)NC3=O)nn(C)c2c1]

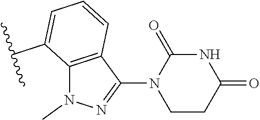 [SMILES: *c1cccc2c(N3CCC(=O)NC3=O)nn(C)c12]

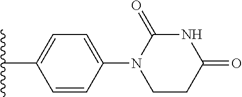 [SMILES: *c1ccc(N2CCC(=O)NC2=O)cc1]

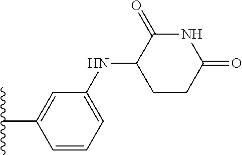 [SMILES: *c1cccc(NC2CCC(=O)NC2=O)c1]

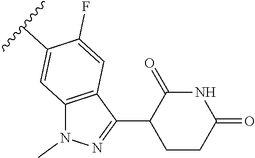 [SMILES: *c1cc2c(cc1F)c(C1CCC(=O)NC1=O)nn2C]

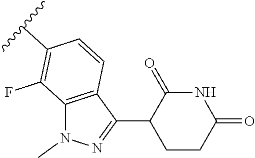 [SMILES: *c1ccc2c(C3CCC(=O)NC3=O)nn(C)c2c1F]

TABLE CM-1a

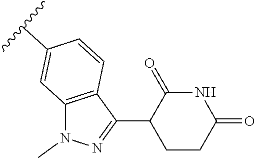 [SMILES: *c1ccc2c(C3CCC(=O)NC3=O)nn(C)c2c1]

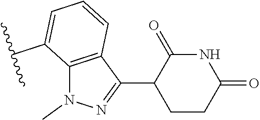 [SMILES: *c1cccc2c(C3CCC(=O)NC3=O)nn(C)c12]

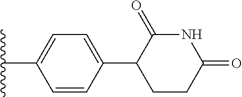 [SMILES: *c1ccc(C2CCC(=O)NC2=O)cc1]

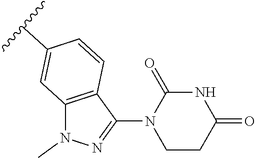 [SMILES: *c1ccc2c(N3CCC(=O)NC3=O)nn(C)c2c1]

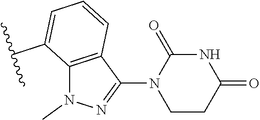 [SMILES: *c1cccc2c(N3CCC(=O)NC3=O)nn(C)c12]

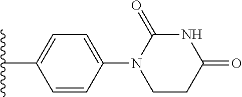 [SMILES: *c1ccc(N2CCC(=O)NC2=O)cc1]

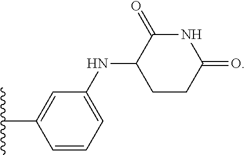 [SMILES: *c1cccc(NC2CCC(=O)NC2=O)c1]

Embodiment 10. The compound of Embodiment 9, wherein

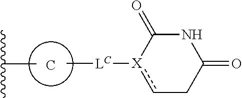

moeity is

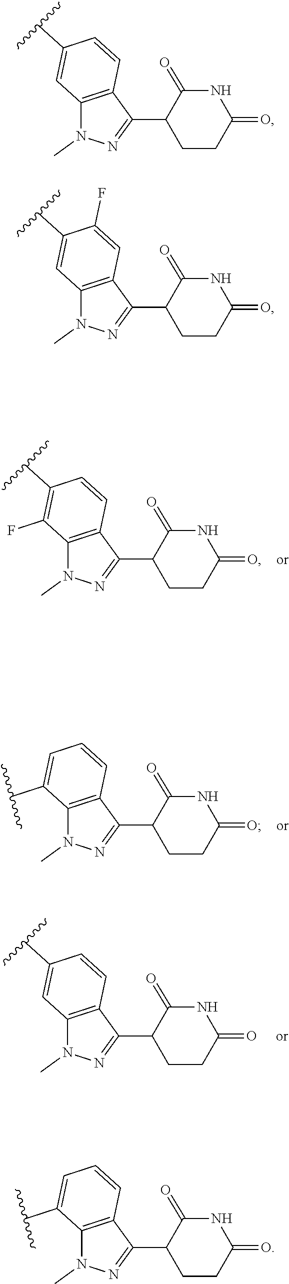 [SMILES: *c1ccc2c(C3CCC(=O)NC3=O)nn(C)c2c1 ; *c1cc2c(cc1F)c(C1CCC(=O)NC1=O)nn2C ; *c1ccc2c(C3CCC(=O)NC3=O)nn(C)c2c1F ; *c1cccc2c(C3CCC(=O)NC3=O)nn(C)c12 ; *c1ccc2c(C3CCC(=O)NC3=O)nn(C)c2c1 ; *c1cccc2c(C3CCC(=O)NC3=O)nn(C)c12]

Embodiment 11. The compound of any one of Embodiments 1-10, wherein -(A¹)ₘ₁- is —C(R⁴R⁴)—CH₂—* (e.g., —CF₂—CH₂—*), wherein * represents the point of attachment to -(A²)ₘ₂-.

Embodiment 12. The compound of any one of Embodiments 1-11, wherein one R³ is C₃₋₆ cycloalkyl; and the other R³ is H.

Embodiment 13. The compound of any one of Embodiments 1-12, wherein one R³ is cyclopropyl; and the other R³ is H.

Embodiment 14. The compound of any one of Embodiments 1-10, wherein one R³ is cyclopropyl; the other R³ is H; and -(A¹)ₘ₁- is —CF₂—CH₂—*, wherein * represents the point of attachment to -(A²)ₘ₂-.

Embodiment 15. The compound of any one of Embodiments 1-14, wherein the carbon atom to which each R³ is attached has (S)-stereochemical configuration.

Embodiment 16. The compound of any one of Embodiments 1-15, wherein X¹ is CH.

Embodiment 17. The compound of any one of Embodiments 1-16, wherein X^{a} is N; and X^{b} is CH.

Embodiment 18. The compound of any one of Embodiments 1-16, wherein X^{a} is CH; and X^{b} is CH.

Embodiment 19. The compound of any one of Embodiments 1-18, wherein R⁶ is halo (e.g., —F, —Cl, —Br) or CN; or R⁶ is halo (e.g., —F, —Cl, —Br).

Embodiment 20. The compound of any one of Embodiments 1-19, wherein R⁶ is —F.

Embodiment 21. The compound of any one of Embodiments 1-19, wherein R⁶ is —Cl.

Embodiment 22. The compound of any one of Embodiments 1-21, wherein each R² is H.

Embodiment 23. The compound of any one of Embodiments 1-22, wherein m3 is 1; and X³ is C₁₋₃ alkylene (e.g., methylene, ethylene, or isopropylene).

Embodiment 24. The compound of any one of Embodiments 1-22, wherein m3 is 0.

Embodiment 25. The compound of any one of Embodiments 1-24, wherein R¹ is H.

Embodiment 26. The compound of any one of Embodiments 1-22, wherein m3 is 1; X³ is C₁₋₃ alkylene optionally substituted with 1-3 R^{c}; and R¹ is H.

Embodiment 27. The compound of any one of Embodiments 1-22, wherein m3 is 1; X³ is C₁₋₃ alkylene; and R¹ is H.

Embodiment 28. The compound of any one of Embodiments 1-22, wherein —(X³)ₘ₃—R¹ is methyl, ethyl, or isopropyl (e.g., methyl).

Embodiment 29. The compound of any one of Embodiments 1-10, wherein TBM is

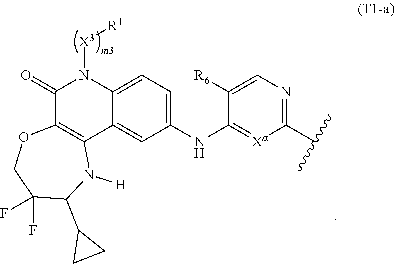

Embodiment 30. The compound of Embodiment 29, wherein m3 is 1; X³ is C₁₋₃ alkylene; and R¹ is H.

Embodiment 31. The compound of Embodiments 29 or 30, wherein —(X³)ₘ₃—R¹ is methyl, ethyl, or isopropyl.

Embodiment 32. The compound of any one of Embodiments 29-31, wherein X^{a} is CH.

Embodiment 33. The compound of any one of Embodiments 29-31, wherein X^{a} is N.

Embodiment 34. The compound of any one of Embodiments 29-33, wherein R⁶ is —F or —Cl.

Embodiment 35. The compound of any one of Embodiments 1-34, wherein L is -(LA)₁₁-, wherein LA and n1 are defined according to (AA).

Embodiment 36. The compound of any one of Embodiments 1-35, wherein n1 is an integer from 1 to 5.

Embodiment 37. The compound of any one of Embodiments 1-36, wherein n1 is an integer from 2 to 4 (e.g., 2 or 3).

Embodiment 38. The compound of Embodiment 1, wherein the compound is selected from the group consisting of Compound No. 101, 101a, 102, 102a, 130, 130a, 132, 132a, 133, 133a, 170, 170a, 186, 186a, 194, 194a, 214, 214a, 214b, 374, 374a, 375, 375a, 379, 379a, 379b, 380, 380a, 380b, 381, 381a, 381b, 382, 382a, 383, 383a, 384, 384a, 385, 385a, 386, 386a, 387, 387a, 388, 388a, 389, 389a, 390, 390a, 391, 391a, 392, 392a, 393, 393a, 394, 394a, 395, 395a, 396, 396a, 397, 397a, 398, 398a, 399, 399a, 400, 400a, 401, 401a, 402, 402a, 403, 403a, 404, 404a, 405, 405a, 406, 406a, 407, 407a, 408, 408a, 409, 409a, 410, 410a, 411, 411a, 412, 412a, 413, 413a, 414, 414a, 415, 415a, 416, 416a, 417, 417a, 418, 418a, 419, 419a, 420, 420a, 421, 421a, 422, 422a, 423, 423a, 424, 424a, 425, 425a, 426, 426a, 426b, 427, 427a, 428, 428a, 429, 429a, 430, 430a, 431, 431a, 432, 432a, 433, 433a, 434, 434a, 435, 435a, 436, 436a, 437, 437a, 438, 438a, 439, 439a, 440, 440a, 441, 441a, 442, 442a, 442b, 443, 443a, 443b, 444, 444a, 445, 445a, 446, 446a, 447, 447a, 448, 448a, 449, 449a, 450, 450a, 451, 451a, 451b, 452, 452a, 453, 453a, 454, 454a, 455, 455a, 456, 456a, 457, 457a, 458, 458a, 459, 459a, 459b, 460, 460a, 460b, 461, 461a, 462, 462a, 463, 463a, 464, 464a, 464b, 464c, 464d, 465, 465a, 465b, 465c, 465d, 466, 466a, 466b, 467, 467a, 468, 468a, 469, 469a, 470, 470a, 471, 471a, 472, 472a, 473, 473a, 474, 474a, 475, 475a, 475b, 476, 476a, 476b, 477, 477a, 478, 478a, 479, 479a, 480, 480a, 481, 481a, 481b, 482, 482a, 482b, 483, 483a, 484, 484a, 485, 485a, 486, 486a, 486b, 486c, 486d, 487, 487a, 487b, 487c, 487d, 488, 488a, 489, 489a, 490, 490a, 491, 491a, 491b, 492, 492a, 492b, 492c, 492d, 493, 493a, 493b, 494, 494a, 495, 495a, 496, 496a, 496b, 497, 497a, 498, 498a, 499, 499a, 499b, 500, 500a, 500b, 501, 501a, 502, 502a, 503, 503a, 504, 504a, 505, 505a, 506, 506a, 506b, 507, 507a, 508, 508a, 509, 509a, 510, 510a, 511, 511a, 511b, 512, 512a, 512b, 513, 513a, 514, 514a, 516, 516a, 518, 518a, 519, 519a, 520, 520a, 521, 521a, 522, 522a, 522b, 523, 523a, 523b, 524, 524a, 525, 525a, 525b, 525c, 525d, 526, 526a, 527, 527a, 527b, 528, 528a, 529, 529a, 529b, 529c, 530, 530a, 531, 531a, 532, 532a, 533, 533a, 534, 534a, 534b, 534c, 534d, 535, 535a, 536, 536a, 537, 537a, 538, 538a, 538b, 538c, 539, 539a, 540, 540a, 541, 541a, 542, 542a, 543, 543a, 544, 544a, 545, 545a, 546, 546a, 547, 547a, 548, 548a, 548b, 549, 549a, 550, 550a, 551, 551a, 552, 552a, 553, 553a, 554, 554a, 555, 555a, 556, 556a, 557, 557a, 558, 558a, 559, 559a, 559b, 560, 560a, 560b, 561, 561a, 562, 562a, 563, 563a, 563b, 564, 564a, 565, 565a, 566, 566a, 568, 568a, 568b, 569, 569a, 570, 570a, 572, 572a, 573, 573a, 574, 574a, 575, 575a, 575b, 576, 576a, 577, 577a, 578, 578a, 578b, 579, 579a, 579b, 581, 581a, 582, 582a, and 582b as depicted in Table C1, or a pharmaceutically acceptable salt thereof; or
Compound No. 101, 101a, 102,102a, 130,130a, 132,132a, 133,133a, 170,170a, 186,186a, 194, 194a, 214, 214a, 214b, 374, 374a, 375, 375a, 379, 379a, 379b, 380, 380a, 380b, 381, 381a, 381b, 382, 382a, 383, 383a, 384, 384a, 385, 385a, 386, 386a, 387, 387a, 388, 388a, 389, 389a, 390, 390a, 391, 391a, 392, 392a, 393, 393a, 394, 394a, 395, 395a, 396, 396a, 397, 397a, 398, 398a, 399, 399a, 400, 400a, 401, 401a, 402, 402a, 403, 403a, 404, 404a, 405, 405a, 406, 406a, 407, 407a, 408, 408a, 409, 409a, 410, 410a, 411, 411a, 412, 412a, 413, 413a, 414, 414a, 415, 415a, 416, 416a, 417, 417a, 418, 418a, 419, 419a, 420, 420a, 421, 421a, 422, 422a, 423, 423a, 424, 424a, 425, 425a, 426, 426a, 426b, 427, 427a, 428, 428a, 429, 429a, 430, 430a, 431, 431a, 432, 432a, 433, 433a, 434, 434a, 435, 435a, 436, 436a, 437, 437a, 438, 438a, 439, 439a, 440, 440a, 441, 441a, 442, 442a, 442b, 443, 443a, 443b, 444, 444a, 445, 445a, 446, 446a, 447, 447a, 448, 448a, 449, 449a, 450, 450a, 451, 451a, 451b, 452, 452a, 453, 453a, 454, 454a, 455, 455a, 456, 456a, 457, 457a, 458, 458a, 459, 459a, 459b, 460, 460a, 460b, 461, 461a, 462, 462a, 463, 463a, 464, 464a, 464b, 464c, 464d, 465, 465a, 465b, 465c, 465d, 466, 466a, 466b, 467, 467a, 468, 468a, 469, 469a, 470, 470a, 471, 471a, 472, 472a, 473, 473a, 474, 474a, 475, 475a, 475b, 476, 476a, 476b, 477, 477a, 478, 478a, 479, 479a, 480, 480a, 481, 481a, 481b, 482, 482a, 482b, 483, 483a, 484, 484a, 485, 485a, 486, 486a, 486b, 486c, 486d, 487, 487a, 487b, 487c, 487d, 488, 488a, 489, 489a, 490, 490a, 491, 491a, 491b, 492, 492a, 492b, 492c, 492d, 493, 493a, 493b, 494, 494a, 495, 495a, 496, 496a, 496b, 497, 497a, 498, 498a, 499, 499a, 500, 500a, 500b, 501, 501a, 502, 502a, 503, 503a, 504, 504a, 505, 505a, 506, 506a, 506b, 507, 507a, 508, 508a, 509, 509a, 510, 510a, 511, 511a, 512, 512a, 512b, 513, 513a, 514, 514a, 516, 516a, 518, 518a, 519, 519a, 520, 520a, 521, 521a, 522, 522a, 522b, 523, 523a, 523b, 524, 524a, 525, 525a, 525b, 525c, 525d, 526, 526a, 527, 527a, 527b, 528, 528a, 529, 529a, 529b, 529c, 530, 530a, 531, 531a, 532, 532a, 533, 533a, 534, 534a, 534b, 534c, 534d, 535, 535a, 536, 536a, 537, 537a, 538, 538a, 538b, 538c, 539, 539a, 540, 540a, 541, 541a, 542, 542a, 543, 543a, 544, 544a, 545, 545a, 546, 546a, 547, 547a, 548, 548a, 548b, 549, 549a, 550, 550a, 551, 551a, 552, 552a, 553, 553a, 554, 554a, 555, 555a, 556, 556a, 557, 557a, 558, 558a, 559, 559a, 559b, 560, 560a, 560b, 561, 561a, 562, 562a, 563, 563a, 563b, 564, 564a, 565, 565a, 566, 566a, 568, 568a, 568b, 569, 569a, 570, 570a, 572, 572a, 573, 573a, 574, 574a, 575, 575a, 575b, 576, 576a, 577, 577a, 578, 578a, 578b, 579, 579a, 579b, 581, 581a, 582, 582a, and 582b as depicted in Table C1, or a pharmaceutically acceptable salt thereof;
Compound No. 101, 101a, 102,102a, 130,130a, 132,132a, 133,133a, 170,170a, 186,186a, 194, 194a, 214, 214a, 214b, 374, 374a, 375, 375a, 379, 379a, 379b, 380, 380a, 380b, 381, 381a, 381b, 382, 382a, 383, 383a, 384, 384a, 385, 385a, 386, 386a, 387, 387a, 388, 388a, 389, 389a, 390, 390a, 391, 391a, 392, 392a, 393, 393a, 394, 394a, 395, 395a, 396, 396a, 397, 397a, 398, 398a, 399, 399a, 400, 400a, 401, 401a, 402, 402a, 403, 403a, 404, 404a, 405, 405a, 406, 406a, 407, 407a, 408, 408a, 409, 409a, 410, 410a, 411, 411a, 412, 412a, 413, 413a, 414, 414a, 415, 415a, 416, 416a, 417, 417a, 418, 418a, 419, 419a, 420, 420a, 421, 421a, 422, 422a, 423, 423a, 424, 424a, 425, 425a, 426, 426a, 426b, 427, 427a, 428, 428a, 429, 429a, 430, 430a, 431, 431a, 432, 432a, 433, 433a, 434, 434a, 435, 435a, 436, 436a, 437, 437a, 438, 438a, 439, 439a, 440, 440a, 441, 441a, 442, 442a, 442b, 443, 443a, 443b, 444, 444a, 445, 445a, 446, 446a, 447, 447a, 448, 448a, 449, 449a, 450, 450a, 451, 451a, 451b, 452, 452a, 453, 453a, 454, 454a, 455, 455a, 456, 456a, 457, 457a, 458, 458a, 459, 459a, 459b, 460, 460a, 460b, 461, 461a, 462, 462a, 463, 463a, 464, 464a, 464b, 464c, 464d, 465, 465a, 465b, 465c, 465d, 466, 466a, 466b, 467, 467a, 468, 468a, 469, 469a, 470, 470a, 471, 471a, 472, 472a, 473, 473a, 474, 474a, 475, 475a, 475b, 476, 476a, 476b, 477, 477a, 478, 478a, 479, 479a, 480, 480a, 481, 481a, 481b, 482, 482a, 482b, 483, 483a, 484, 484a, 485, 485a, 486, 486a, 486b, 486c, 486d, 487, 487a, 487b, 487c, 487d, 488, 488a, 489, 489a, 490, 490a, 491, 491a, 491b, 492, 492a, 492b, 492c, 492d, 493, 493a, 493b, 494, 494a, 495, 495a, 496, 496a, 496b, 497, 497a, 498, 498a, 499, 499a, 500, 500a, 500b, 501, 501a, 502, 502a, 503, 503a, 504, 504a, 505, 505a, 506, 506a, 506b, 507, 507a, 508, 508a, 509, 509a, 510, 510a, 511, 511a, 512, 512a, 512b, 513, 513a, 514, 514a, 516, 516a, 518, 518a, 519, 519a, 520, 520a, 521, 521a, 522, 522a, 522b, 523, 523a, 523b, 524, 524a, 525, 525a, 525b, 525c, 525d, 526, 526a, 527, 527a, 527b, 528, 528a, 529, 529a, 529b, 529c, 530, 530a, 531, 531a, 532, 532a, 533, 533a, 534, 534a, 534b, 534c, 534d, 535, 535a, 536, 536a, 537, 537a, 538, 538a, 538b, 539, 539a, 540, 540a, 541, 541a, 542, 542a, 543, 543a, 544, 544a, 545, 545a, 546, 546a, 547, 547a, 548, 548a, 548b, 549, 549a, 550, 550a, 551, 551a, 552, 552a, 553, 553a, 554, 554a, 555, 555a, 556, 556a, 557, 557a, 558, 558a, 559, 559a, 559b, 560, 560a, 560b, 561, 561a, 562, 562a, 563, 563a, 563b, 564, 564a, 565, 565a, 566, and 566a as depicted in Table C1, or a pharmaceutically acceptable salt thereof; or
Compound No. 101, 101a, 102, 102a, 130, 130a, 132, 132a, 133, 133a, 170, 170a, 186, 186a, 194, 194a, 214, 214a, 214b, 374, 374a, 375, 375a, 379, 379a, 379b, 380, 380a, 380b, 381, 381a, 381b, 382, 382a, 383, 383a, 384, 384a, 385, 385a, 386, 386a, 387, 387a, 388, 388a, 389, 389a, 390, 390a, 391, 391a, 392, 392a, 393, 393a, 394, 394a, 395, 395a, 396, 396a, 397, 397a, 398, 398a, 399, 399a, 400, 400a, 401, 401a, 402, 402a, 403, 403a, 404, 404a, 405, 405a, 406, 406a, 407, 407a, 408, 408a, 409, 409a, 410, 410a, 411, 411a, 412, 412a, 413, 413a, 414, 414a, 415, 415a, 416, 416a, 417, 417a, 418, 418a, 419, 419a, 420, 420a, 421, 421a, 422, 422a, 423, 423a, 424, 424a, 425, 425a, 426, 426a, 426b, 427, 427a, 428, 428a, 429, 429a, 430, 430a, 431, 431a, 432, 432a, 433, 433a, 434, 434a, 435, 435a, 436, 436a, 437, 437a, 438, 438a, 439, 439a, 440, 440a, 441, 441a, 442, 442a, 442b, 443, 443a, 443b, 444, 444a, 445, 445a, 446, 446a, 447, 447a, 448, 448a, 449, 449a, 450, 450a, 451, 451a, 451b, 452, 452a, 453, 453a, 454, 454a, 455, 455a, 456, 456a, 457, 457a, 458, 458a, 459, 459a, 459b, 460, 460a, 460b, 461, 461a, 462, 462a, 463, 463a, 464, 464a, 464b, 464c, 464d, 465, 465a, 465b, 465c, 465d, 466, 466a, 466b, 467, 467a, 468, 468a, 469, 469a, 470, 470a, 471, 471a, 472, 472a, 473, 473a, 474, 474a, 475, 475a, 475b, 476, 476a, 476b, 477, 477a, 478, 478a, 479, 479a, 480, 480a, 481, 481a, 481b, 482, 482a, 482b, 483, 483a, 484, 484a, 485, 485a, 486, 486a, 486b, 486c, 486d, 487, 487a, 487b, 487c, 487d, 488, 488a, 489, 489a, 490, 490a, 491, 491a, 491b, 492, 492a, 493, 493a, 494, 494a, 495, 495a, 496, 496a, 496b, 497, 497a, 498, 498a, 499, 499a, 500, 500a, and 500b as depicted in Table C1, or a pharmaceutically acceptable salt thereof; or
Compound No. 101, 101a, 102, 102a, 130, 130a, 132, 132a, 214, 214a, 214b, 374, 374a, 379, 379a, 379b, 380, 380a, 380b, 381, 381a, 381b, 382, 382a, 383, 383a, 384, 384a, 385, 385a, 386, 386a, 387, 387a, 388, 388a, 389, 389a, 390, 390a, 391, 391a, 392, 392a, 393, 393a, 394, 394a, 395, 395a, 396, 396a, 397, 397a, 398, 398a, 399, 399a, 400, 400a, 401, 401a, 402, 402a, 403, 403a, 404, 404a, 405, 405a, 406, 406a, 407, 407a, 408, 408a, 409, 409a, 410, 410a, 411, 411a, 412, 412a, 413, 413a, 414, 414a, 415, 415a, 416, and 416a as depicted in Table C1, or a pharmaceutically acceptable salt thereof.

Embodiment 39. A pharmaceutical composition comprising a compound of any one of Embodiments 1-38, or a pharmaceutically acceptable salt thereof, and a pharmaceutically acceptable excipient.

Embodiment 40. A compound of any one of Embodiments 1-38, or a pharmaceutically acceptable salt thereof, or the pharmaceutical composition of Embodiment 39 for use in treatment.

Embodiment 41. A compound of any one of Embodiments 1-38 or a pharmaceutically acceptable salt thereof, or the pharmaceutical composition of Embodiment 39 for use in the treatment of a cancer selected from the group consisting of a hematological cancer, breast cancer, gastrointestinal cancer, brain cancer, and lung cancer.

Embodiment 42. The compound or pharmaceutical composition of Embodiment 41, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML); optionally wherein the hematological cancer is selected from the group consisting of: is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), or chronic myeloid leukemia (CML); optionally wherein the hematological cancer is selected from the group consisting of DLBCL, FL, MCL, BL, PTCL, and ALL (e.g., B-ALL); optionally wherein the hematological cancer is FL or DLBCL; optionally where the hematological cancer is DLBCL.

Embodiment 43. The compound or pharmaceutical composition of Embodiment 40 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof for use in treatment; or an additional therapeutic agent or therapy selected from the group consisting of is a PI3K inhibitor, a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof for use in treatment.

Embodiment 44. The compound or pharmaceutical composition of Embodiment 42 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or an additional therapeutic agent or therapy selected from the group consisting of is a PI3K inhibitor, a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof.

Embodiment 45. A compound of any one of Embodiments 1-38, or a pharmaceutically acceptable salt thereof, or the pharmaceutical composition of Embodiment 39 for use in a method of treating cancer selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML); optionally wherein the cancer is selected from the group consisting of: is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), or chronic myeloid leukemia (CML); optionally wherein the cancer is selected from the group consisting of DLBCL, FL, MCL, BL, PTCL, and ALL (e.g., B-ALL); optionally wherein the cancer is FL or DLBCL; optionally where the cancer is DLBCL, the method comprising administering to a subject a therapeutically effective amount of a compound of Embodiments 1-38, or a pharmaceutically acceptable salt thereof, or the pharmaceutical composition of Embodiment 39.

Embodiment 46. The compound or pharmaceutical composition of Embodiment 45, wherein the method comprises administering to the subject a therapeutically effective amount of an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or an additional therapeutic agent or therapy selected from the group consisting of is a PI3K inhibitor, a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof.

Embodiment 47. The compound or pharmaceutical composition of Embodiment 46, wherein the compound of any one of Embodiments 1-38, or a pharmaceutically acceptable salt thereof, or the pharmaceutical composition of Embodiment 39, and the additional therapeutic agent or therapy are administered simultaneously, separately, or sequentially.

What is claimed is:

1. A compound of Formula (I): 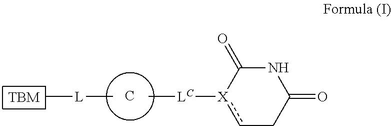
or a pharmaceutically acceptable salt thereof, wherein:
TBM is (T1): 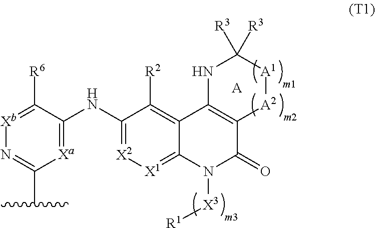
X¹ is selected from the group consisting of N and CR²;
X² is CH;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
m3 is 0 or 1;
X³ is C₁₋₃ alkylene optionally substituted with 1-3 R^{e};
R¹ is selected from the group consisting of: H and C₃₋₆ cycloalkyl;
m1 is 2; each A¹ is independently CH₂, CHR⁴, or CR⁴R⁴;
m2 is 1; A² is —O—;
one R³ is selected from the group consisting of:
(i) C₃₋₆ cycloalkyl optionally substituted with 1-3 R⁹, and
(ii) C₁₋₃ alkyl optionally substituted with 1-3 —F; and
the other R³ is H;
each R⁴ is independently selected from the group consisting of: H, R^{a}, and R^{b};
X^{a} is selected from the group consisting of: N, CH, and CF;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):
(AA)
n1 is an integer from 1 to 15; and
each L^{A} is independently selected from the group consisting of: L^{A1} L^{A3}, and L^{A4} provided that 1-3 occurrences of L^{A} is L^{A4};
(BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any 0-0, N-0, N—N, N—S(O)o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{e};
Ring C is selected from the group consisting of: 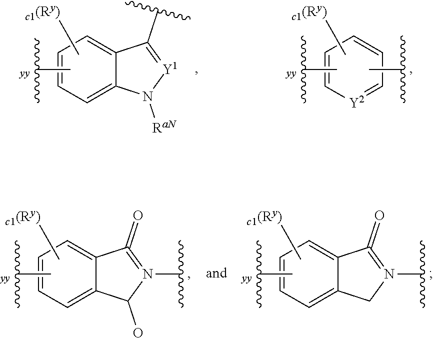
c1 is 0, 1, 2, or 3;
each R^{Y} is independently selected from the group consisting of: R^{a} and R^{b};
R^{a}N is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than 0; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl;
(h) C(═O)C₁₋₆ haloalkyl;
(i) C(═O)OH;
(j) C(═O)OC₁₋₆ alkyl;
(k) C(═O)OC₁₋₆ haloalkyl;
(l) C(═O)N(R^{f})₂;
(m) S(O)₀₋₂(C₁₋₆ alkyl);
(n) S(O)₀₋₂(C₁₋₆ haloalkyl);
(o) S(O)₁₋₂N(R)₂; and
(p) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{e} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

2. The compound of claim 1, wherein Ring C is 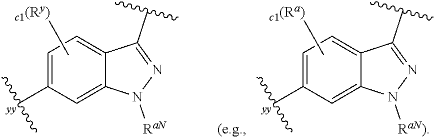
or
Ring C is

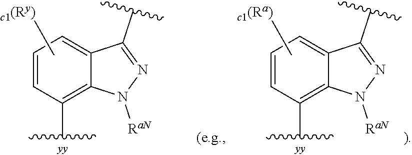

3. The compound of any one of claims 1 or 2, wherein c1 is 0; or
c1 is 1; and R¹ is halo (e.g., —F).

4. The compound of any one of claims 1-3, wherein R^{aN} is C₁₋₃ alkyl (e.g., methyl).

5. The compound of any one of claims 1-4, wherein X is CH.

6. The compound of any one of claims 1-5, wherein L^{C} is a bond.

7. The compound of claim 1, wherein the 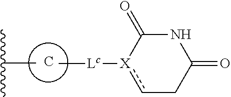
moiety is selected from the group consisting of the moieties delineated in Table CM-1b:
TABLE CM-1b

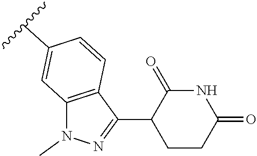 [SMILES: *c1ccc2c(C3CCC(=O)NC3=O)nn(C)c2c1]

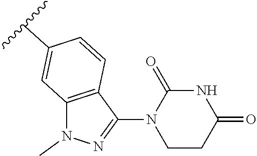 [SMILES: *c1ccc2c(N3CCC(=O)NC3=O)nn(C)c2c1]

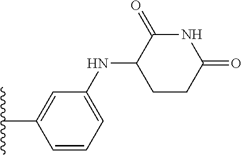 [SMILES: *c1cccc(NC2CCC(=O)NC2=O)c1]

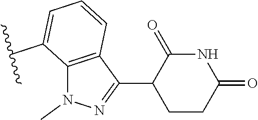 [SMILES: *c1cccc2c(C3CCC(=O)NC3=O)nn(C)c12]

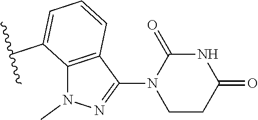 [SMILES: *c1cccc2c(N3CCC(=O)NC3=O)nn(C)c12]

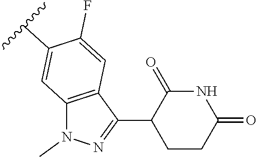 [SMILES: *c1cc2c(cc1F)c(C1CCC(=O)NC1=O)nn2C]

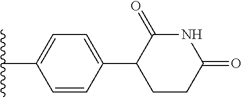 [SMILES: *c1ccc(C2CCC(=O)NC2=O)cc1]

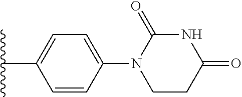 [SMILES: *c1ccc(N2CCC(=O)NC2=O)cc1]

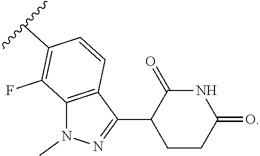 [SMILES: *c1ccc2c(C3CCC(=O)NC3=O)nn(C)c2c1F]

8. The compound of claim 7, wherein 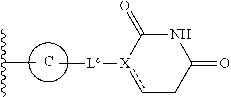
moiety is 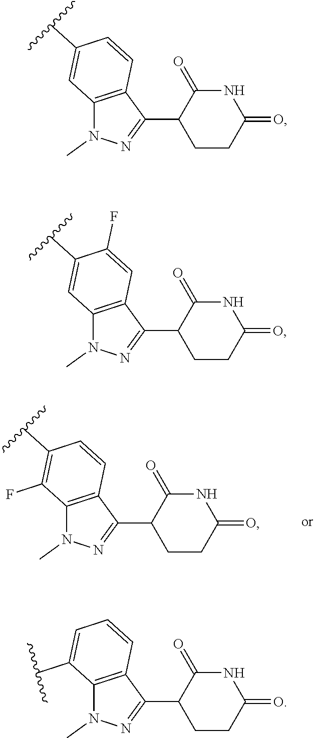 [SMILES: *c1ccc2c(C3CCC(=O)NC3=O)nn(C)c2c1 ; *c1cc2c(cc1F)c(C1CCC(=O)NC1=O)nn2C ; *c1ccc2c(C3CCC(=O)NC3=O)nn(C)c2c1F ; "or" ; *c1cccc2c(C3CCC(=O)NC3=O)nn(C)c12]
or wherein 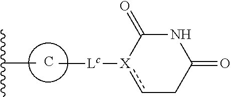
moiety is

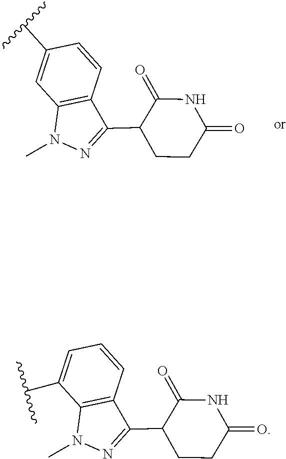 [SMILES: *c1ccc2c(C3CCC(=O)NC3=O)nn(C)c2c1 ; *c1cccc2c(C3CCC(=O)NC3=O)nn(C)c12]

9. The compound of any one of claims 1-8, wherein -(A¹)ₘ₁- is —C(R⁴R⁴)—CH₂—* (e.g., —CF₂—CH₂—*), wherein * represents the point of attachment to -(A²))ₘ₂-.

10. The compound of any one of claims 1-9, wherein one R³ is C₃₋₆ cycloalkyl; and the other R³ is H; or
one R³ is cyclopropyl; and the other R³ is H; or
one R³ is cyclopropyl; the other R³ is H; and -(A¹)ₘ₁- is —CF₂—CH₂—*, wherein * represents the point of attachment to -(A²)ₘ₂-.

11. The compound of any one of claims 1-10, wherein the carbon atom to which each R³ is attached has (S)-stereochemical configuration.

12. The compound of any one of claims 1-11, wherein X¹ is CH.

13. The compound of any one of claims 1-12, wherein X^{a} is N; and X^{b} is CH; or
X^{a} is CH; and X^{b} is CH.

14. The compound of any one of claims 1-13, wherein R⁶ is halo (e.g., —F, —Cl, —Br) or CN; or
R⁶ is —F; or
R⁶ is —Cl.

15. The compound of any one of claims 1-14, wherein each R² is H.

16. The compound of any one of claims 1-15, wherein m3 is 1; and X³ is C₁₋₃ alkylene (e.g., methylene, ethylene, or isopropylene); or
m3 is 0.

17. The compound of any one of claims 1-16, wherein R¹ is H.

18. The compound of any one of claims 1-15, wherein m3 is 1; X³ is C₁₋₃ alkylene optionally substituted with 1-3 R^{c}; and R¹ is H; or
m3 is 1; X³ is C₁₋₃ alkylene; and R¹ is H.

19. The compound of any one of claims 1-15, wherein —(X³)ₘ₃—R¹ is methyl, ethyl, or isopropyl (e.g., methyl).

20. The compound of any one of claims 1-8, wherein TBM is

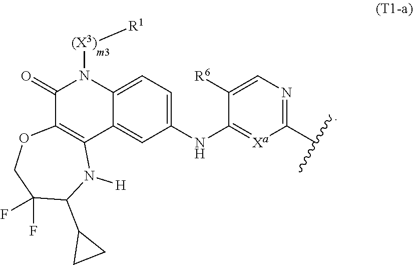

21. The compound of claim 20, wherein m3 is 1; X³ is C₁₋₃ alkylene; and R¹ is H.

22. The compound of claims 20 or 21, wherein —(X³)ₘ₃—R¹ is methyl, ethyl, or isopropyl.

23. The compound of any one of claims 20-22, wherein X^{a} is CH; or
X^{a} is N.

24. The compound of any one of claims 20-23, wherein R⁶ is —F or —C_{1.}

25. The compound of any one of claims 1-24, wherein L is -(L^{A})ₙ₁-, wherein L^{A} and n1 are defined according to (AA).

26. The compound of any one of claims 1-25, wherein n1 is an integer from 1 to 5; or
n1 is an integer from 2 to 4 (e.g., 2 or 3).

27. The compound of any one of claims 1-26, wherein L is selected from the group consisting of:
L^{A4}-L^{A1}-L^{A4}-_{bb};
L^{A4}-L^{A4}-_{bb};
L^{A4}-L^{A1}-L^{A1}-L^{A4}-_{bb};
L^{A4}-L^{A3}-L^{A4}-_{bb}; and
L^{A4}-L^{A1}-L^{A4}-L^{A3}-_{bb},
wherein bb represents the point of attachment to Ring C.

28. The compound of claim 27, wherein each L^{A4} is independently a C3-10 cycloalkylene or a 4-12 membered heterocyclylene, each of which is optionally substituted with 1-6 R^{a}; or
each L^{A4} is independently a 4-12 (e.g., 4-10) membered heterocyclylene optionally substituted with 1-3 R^{a}; or
each L^{A4} is independently a monocyclic 4-6 membered nitrogen-containing heterocyclylene optionally substituted with 1-3 R^{a}; or
one L^{A4} is a monocyclic 4-6 membered nitrogen-containing heterocyclylene optionally substituted with 1-3 R^{a}; and
the other L^{A4} is a bicyclic 6-12 (e.g., 6-10) membered nitrogen-containing heterocyclylene optionally substituted with 1-3 R^{a}; or
one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a}; or
each L^{A4} contains 1-2 ring nitrogen atoms and no additional ring heteroatoms.

29. The compound of claim 28, wherein each R^{a} present on L^{A4} is independently selected from the group consisting of: —F, CN, C₁₋₃ alkoxy, OH, and C₁₋₃ alkyl optionally substituted with 1-3 F.

30. The compound of claim 28, wherein L is -L^{A4}-L^{A1}-L^{A4}-_{bb}.

31. The compound of claim 30, wherein L^{A1} is —CH₂—, —CHMe-, or —CMe₂-.

32. The compound of claim 28, wherein L is -L^{A4}-L^{A3}-L^{A4}-_{bb}.

33. The compound of claim 32, wherein L^{A3} is —C(═O).

34. The compound of claim 28 or 29, wherein L is -L^{A4}-L^{A1}-L^{A4}-L^{A3}-_{bb}, and L^{A3} is C(═O).

35. The compound of claim 28, wherein L is -L^{A4}-L^{A1}-L^{A1}-L^{A4}-_{bb}, and one or both of L^{A1} is CH₂.

36. The compound of any one of claims 1-26, wherein L is selected from the group consisting of:
L^{A4}-L^{A3}-_{bb};
L^{A4}-L^{A1}-bb; and
L^{A4}-L^{A1}-L^{A3}-_{bb},
wherein bb represents the point of attachment to Ring C.

37. The compound of claim 36, wherein L is -L^{A4}-L^{A3}-_{bb}.

38. The compound of claims 36 or 37, wherein L^{A3} is —NH— or —N(C₁₋₃ alkyl)- (e.g., —NH—).

39. The compound of any one of claims 36-38, wherein L^{A4} is a 4-12 membered heterocyclylene optionally substituted with 1-6 R^{a}; or
L^{A4} is a 4-12 (e.g., 6-10) membered nitrogen-containing heterocyclylene optionally substituted with 1-3 R^{a}; or
L^{A4} is a bicyclic spirocyclic 6-12 (e.g., 6-10) membered nitrogen-containing heterocyclylene optionally substituted with 1-3 R^{a}; or
L^{A4} contains 1-2 ring nitrogen atoms and no additional ring heteroatoms.

40. The compound of claim 39, wherein each R^{a} present on L^{A4} is independently selected from the group consisting of: —F, CN, C₁₋₃ alkoxy, OH, and C₁₋₃ alkyl optionally substituted with 1-3 F.

41. The compound of any one of claims 1-27, wherein L is selected from the group consisting of the moieties delineated in Table L or Table L1-a, wherein bb represents the point of attachment to Ring C.

42. The compound of claim 1, wherein the compound is a compound of Formula (I-aa): 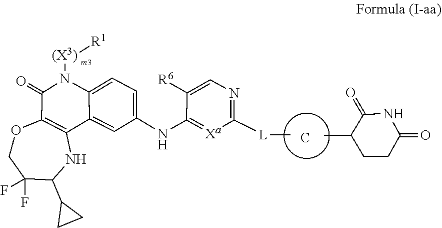
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 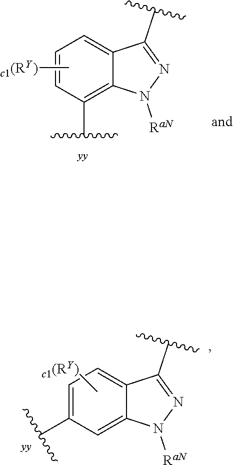
wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:
L^{A4}-L^{A1}-L^{A4}-_{bb};
L^{A4}-L^{A1}-L^{A1}-L^{A4}-_{bb};
L^{A4}-L^{A4}-_{bb};
L^{A4}-C(═O)-L^{A4}-_{bb}; and
L^{A4}-L^{A1}-L^{A4}-C(═O)-_{bb},
wherein bb represents the point of attachment to Ring C; and
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; or
the compound is a compound of Formula (I-a): 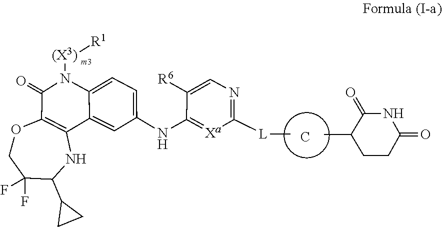
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 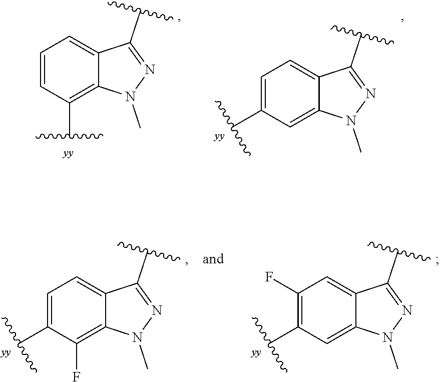 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]
L is selected from the group consisting of:
L^{A4}-L^{A1}-L^{A4}-_{bb};
L^{A4}-L^{A1}-L^{A1}-L^{A4}-_{bb};
L^{A4}-L^{A4}-_{bb};
L^{A4}-C(═O)-L^{A4}-_{bb}; and
L^{A4}-L^{A1}-L^{A4}-C(═O)-_{bb},
wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; or
the compound is a compound of Formula (I-aa-1): 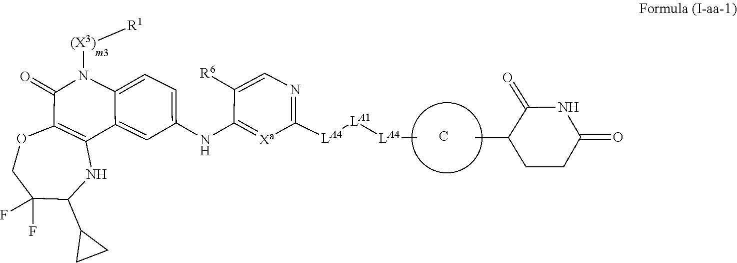
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 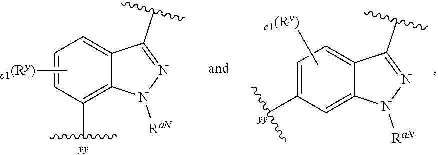
wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F; or
the compound is a compound of Formula (I-a-1): 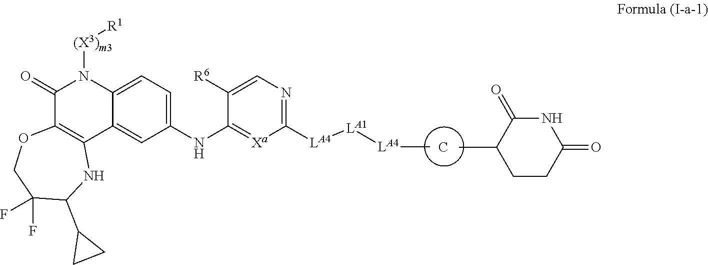
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 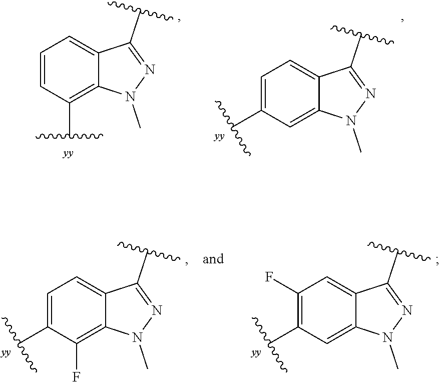 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]
L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F; or
the compound is a compound of Formula (I-aa-2): 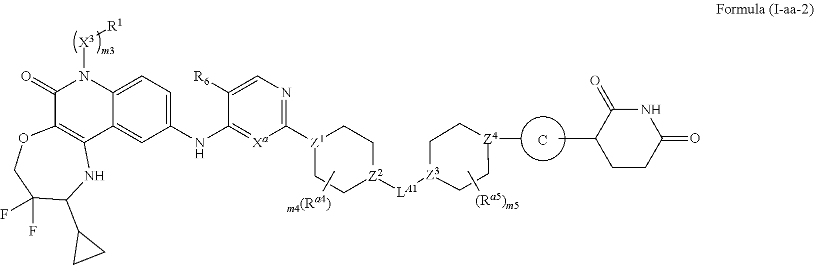
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 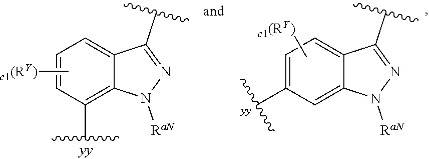
wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F; or
the compound is a compound of Formula (I-a-2): 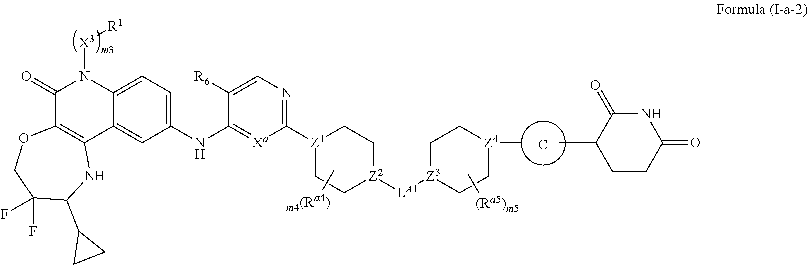
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 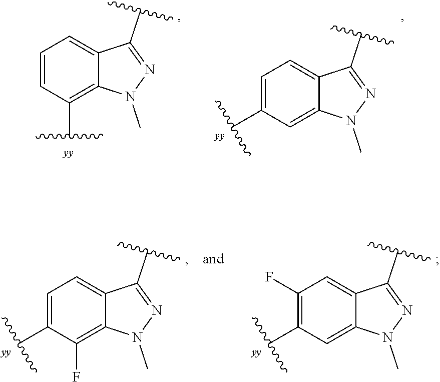 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C1-3 alkoxy, OH, and C₁₋₃ alkyl optionally substituted with 1-3 F; or
the compound is a compound of Formula (I-aa-3): 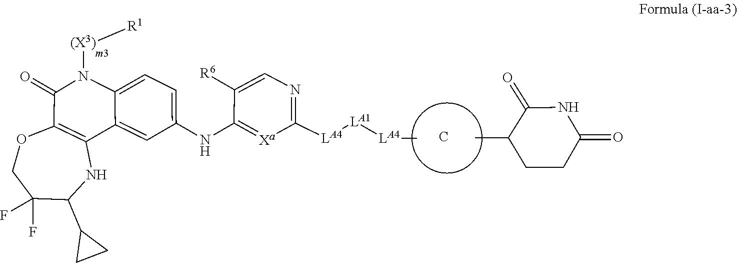
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 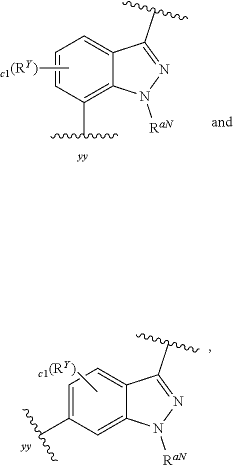
wherein: c1 is 0 or 1, R^{Y} is selected from the group consisting of halo (e.g., —F) and C1-3 alkyl optionally substituted with 1-3 F, and R^{aN} is C₁₋₃ alkyl;
L^{A1} is CH₂, CHMe, or CMe₂; and
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; or
the compound is a compound of Formula (I-a-3): 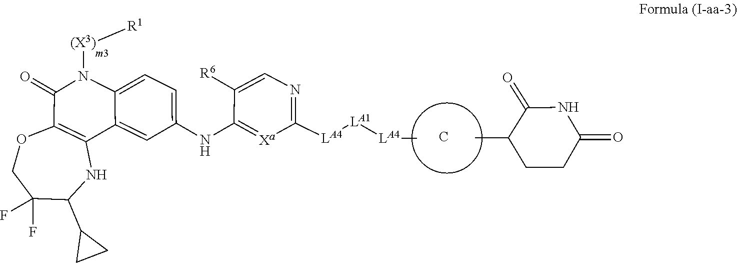
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 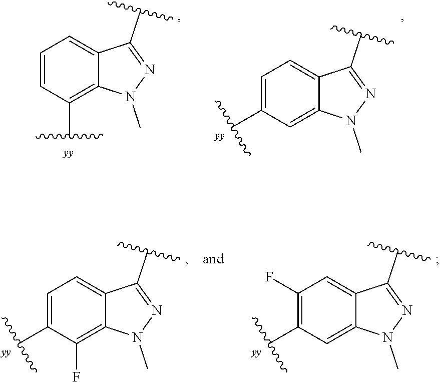 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]
L^{A1} is CH₂, CHMe, or CMe₂;
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; or
the compound is a compound of Formula (I-aa-4): 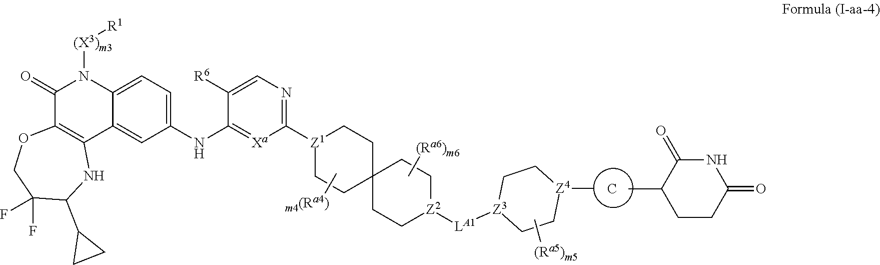
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 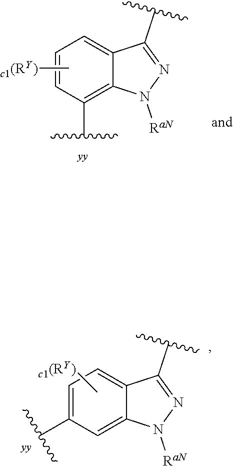
wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F; or
the compound is a compound of Formula (I-a-4): 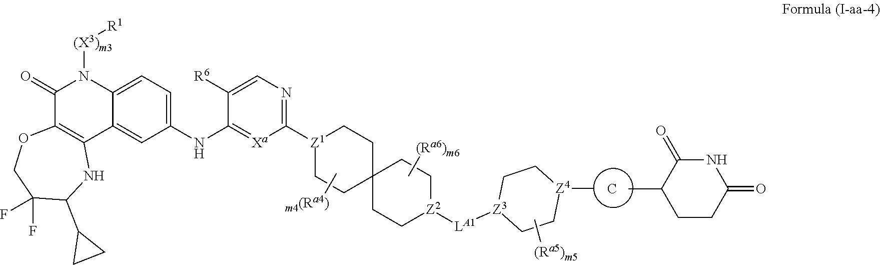
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 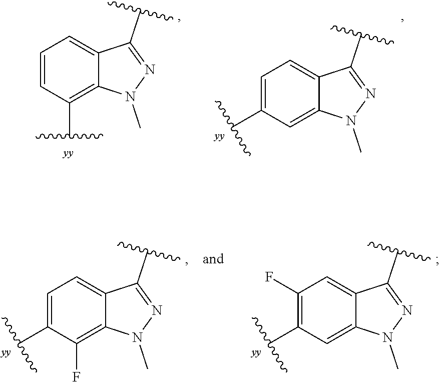 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5};
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F; or
the compound is a compound of Formula (I-bb): 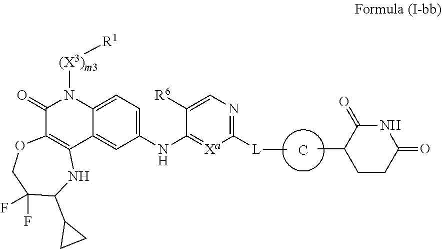
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 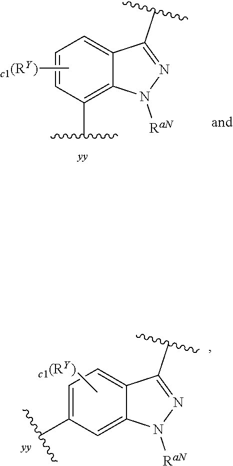
wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is -L^{A4}-L^{A3}-_{bb} or -L^{A4}-L^{A1}-L^{A3}-_{bb}, wherein bb represents the point of attachment to Ring C; and
L^{A4} is a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; or
the compound is a compound of Formula (I-b): 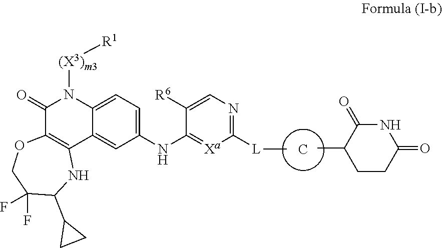
or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of: 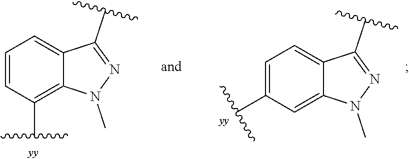 [SMILES: *c1nn(C)c2c(*)cccc12 ; *c1ccc2c(*)nn(C)c2c1]
L is -L^{A4}-L^{A3}-_{bb} or -L^{A4}-L^{A1}-L^{A3}-_{bb}, wherein bb represents the point of attachment to Ring C; and
L^{A4} is 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

43. The compound of claim 1, wherein the compound is selected from the group consisting of the compounds in Table C1, or a pharmaceutically acceptable salt thereof.

44. A pharmaceutical composition comprising a compound of any one of claims 1-43, or a pharmaceutically acceptable salt thereof, and a pharmaceutically acceptable excipient.

45. A method for treating a cancer in a subject in need thereof, the method comprising administering to the subject a therapeutically effective amount of a compound of any one of claims 1-43, or a pharmaceutically acceptable salt thereof, or a pharmaceutical composition according to claim 44.

46. The method of claim 45, wherein the cancer is a hematological cancer, breast cancer, gastrointestinal cancer, brain cancer, lung cancer, or a combination thereof.

47. The method of claim 46, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML).

48. The method of claim 47, wherein the hematological cancer is selected from the group consisting of DLBCL, FL, MCL, BL, PTCL, and ALL (e.g., B-ALL); or
the hematological cancer is FL or DLBCL; or
the hematological cancer is DLBCL; or
the hematological cancer is FL; or
the hematological cancer is BL; or
the hematological cancer is a PTCL; or
the hematological cancer is ALL (e.g., B-ALL).

49. The method of any one of claims 46-48, wherein the therapeutically effective amount of a compound of any one of claims 1-43, or a pharmaceutically acceptable salt thereof, or a pharmaceutical composition according to claim 44, is administered to the subject as a monotherapy.

50. The method of any one of claims 46-48, comprising administering an additional therapy or therapeutic agent to the subject.

51. The method of claim 50, wherein the additional therapy or therapeutic agent is a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-X_{L} inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, or a combination thereof.

52. A method for treating an autoimmune condition in a subject in need thereof, the method comprising administering to the subject a therapeutically effective amount of a compound of any one of claims 1-43, or a pharmaceutically acceptable salt thereof, or a pharmaceutical composition according to claim 44.

53. A method for treating a lymphoproliferative disorder in a subject in need thereof, the method comprising administering to the subject a therapeutically effective amount of a compound of any one of claims 1-43, or a pharmaceutically acceptable salt thereof, or a pharmaceutical composition according to claim 44.

54. A compound of Formula (SI): 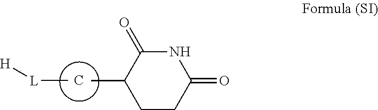
or salts thereof, wherein:
Ring C is selected from the group consisting of: 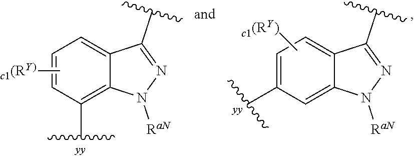
wherein: c1 is 0 or 1, R^{Y} is selected from the group consisting of halo (e.g., —F) and C₁₋₃ alkyl optionally substituted with 1-3 F, and R^{aN} is C₁₋₃ alkyl, and yy represents the point of attachment to L;
L is selected from the group consisting of:
L^{A4}-L^{A1}-L^{A4}-_{bb};
L^{A4}-L^{A1}-L^{A1}-L^{A4}-_{bb};
L^{A4}-L^{A4}-_{bb};
L^{A4}-C(═O)-L^{A4}-_{bb}; and
L^{A4}-L^{A1}-L^{A4}-C(═O)-_{bb},
wherein bb represents the point of attachment to Ring C; and
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; or
a compound of Formula (SI-A) or Formula (SI-B): 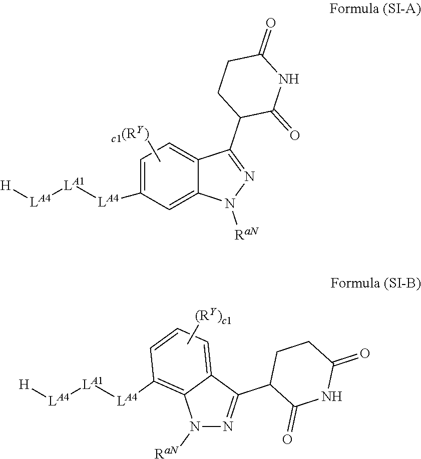
or salts thereof, wherein:
c1 is 0 or 1;
R^{Y} is selected from the group consisting of halo (e.g., —F) and C₁₋₃ alkyl optionally substituted with 1-3 F;
R^{aN} is C₁₋₃ alkyl;
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; or
a compound of Formula (SI-A-1) and Formula (SI-B-1): 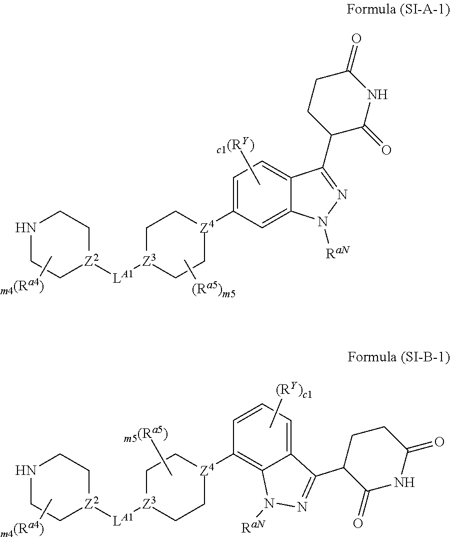
or salts thereof, wherein:
c1 is 0 or 1;
R^{Y} is selected from the group consisting of halo (e.g., —F) and C₁₋₃ alkyl optionally substituted with 1-3 F;
R^{aN} is C₁₋₃ alkyl;
L^{A1} is CH₂, CHMe, or CMe₂;
Z², Z³, and Z⁴ are independently selected from the group consisting of: CH, CR^{a4}, and N, provided that at least one of Z³ and Z⁴ is N; when Z² is N, then Z³ is CH or CR^{a4}; and when Z³ is N, then Z² is CH or CR^{a4};
m4 and m5 are independently 0, 1, or 2; and
each R^{a4} and R^{a5} is independently selected from the group consisting of: —F, CN, C₁₋₃ alkoxy, OH, and C₁₋₃ alkyl optionally substituted with 1-3 F; or
a compound of Formula (SI-A-2) or Formula (SI-B-2): 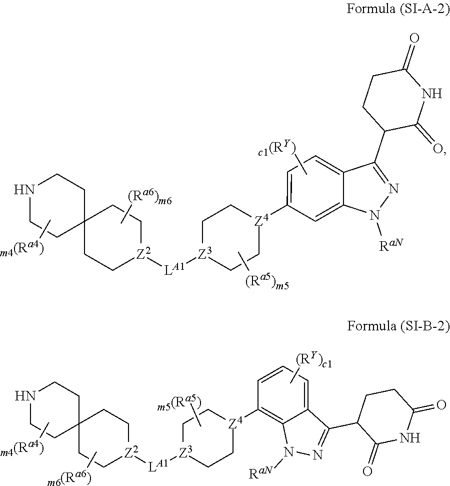
or salts thereof, wherein:
c1 is 0 or 1;
R^{Y} is selected from the group consisting of halo (e.g., —F) and C₁₋₃ alkyl optionally substituted with 1-3 F;
R^{aN} is C₁₋₃ alkyl;
L^{A1} is CH₂, CHMe, or CMe₂;
Z², Z³, and Z⁴ are independently selected from the group consisting of: CH, CR^{a4}, and N, provided that at least one of Z³ and Z⁴ is N; when Z² is N, then Z³ is CH or CR^{a4}; and when Z³ is N, then Z² is CH or CR^{a4};
m4, m5, and m6 are independently 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

55. Any of the compounds, compositions, combinations, pharmaceutical compositions, methods, uses, and processes as substantially provided herein.


SEQUENCE LISTING (1 sequence)

SEQ ID NO: 1 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP